(19) United States
(12) Patent Application Publication
(10) Pub. No.: US 2011/0288155 A1
(43) Pub. Date: Nov. 24, 2011

(54) SIRNA COMPOUNDS AND METHODS OF USE THEREOF

(76) Inventors: Elena Feinstein, Rehovot (IL); Igor Mett, Rehovot (IL); Hagar Kalinski, Rishon-le-Zion (IL)

(21) Appl. No.: 13/140,790

(22) PCT Filed: Dec. 17, 2009
(86) PCT No.: PCT/US09/68342

§ 371 Date: Aug. 3, 2011

Related U.S. Application Data
(60) Provisional application No. 61/203,310, filed on Dec. 18, 2008.
(60) Provisional application No. 61/204,053, filed on Dec. 30, 2008.

(51) Int. Cl. A61K 31/713; A61P 25/28; A61P 11/00; C07H 21/04; A61P 9/00; A61P 27/16; A61P 9/10; C07H 21/02; A61P 27/02

(52) U.S. Cl. 514/44 A; 536/24.5

(57) ABSTRACT

The present application relates to double stranded oligonucleotide inhibitors of target genes, pharmaceutical compositions comprising same and the use of such molecules to treat, inter alia, neurodegenerative disorders including Alzheimer's disease and Amyotrophic Lateral Sclerosis, eye diseases including glaucoma and ION, acute renal failure, hearing loss, acute respiratory distress syndrome and in preventing or treating ischemia-reperfusion injury in organ transplant patients.

### RELATED APPLICATIONS

The present application claims priority from U.S. Application Nos. 61/203,310 filed Dec. 18, 2008 and 61/204,053 filed Dec. 30, 2008, the contents of which are incorporated by reference herein in their entirety.

### FIELD OF THE INVENTION

The present application relates to double stranded oligonucleotide inhibitors of target gene expression including the NADPH oxidase (NOX) genes, pharmaceutical compositions comprising same and methods of use thereof. The compounds and compositions are thus useful in the treatment of subjects suffering from diseases or conditions and or symptoms associated with such diseases or conditions in which expression of one or more target genes has adverse consequences in mammalians. In particular embodiments, the application provides chemically modified siRNA oligonucleotides, compositions comprising same and the use of such molecules to treat, inter alia, neurodegenerative disorders including Alzheimer's disease and Amyotrophic Lateral Sclerosis, eye diseases including glaucoma and ION, acute renal failure, hearing loss, acute respiratory distress syndrome and to prevent injury and complications in transplant patients.

### BACKGROUND OF THE INVENTION

International Patent Publication No. WO 2005/119251 discloses a method of inhibiting NOX3 for treating hearing loss. International Patent Publication No. WO 2002/030453 discloses NADPH oxidase inhibitors for reducing angiogenesis. U.S. Pat. No. 6,846,672 and related patents and patent applications disclose the polynucleotide and polypeptide sequences of the NOX enzymes. US Patent Publication No. 2007/0037883 relates to NOX4 inhibition.

U.S. Pat. Nos. 6,846,672; 7,029,673; 7,202,052; 7,202,053 and 7,226,769 disclose NOX enzymes and regulators thereof. International Patent Publication No. WO 2008/050329 assigned to the assignee of the present application, hereby incorporated by reference in its entirety, relates to certain NOX genes. International Patent Application Nos. WO 2008/104978 and WO 2009/044392 assigned to the assignee of the current application and hereby incorporated by reference in their entirety relate to novel siRNA structures. In particular, Publication No. WO 2009/044392 relates to siRNA oligonucleotides targetting some NOX-related genes useful in preparing siRNA compounds.

International Patent Publication No. WO 2008/152636 assigned to the assignee of the present application and hereby incorporated by reference in its entirety discloses siRNA compounds to the NOX genes, to compositions comprising same and to methods of use thereof.

International Patent Publication No. WO 00/44364 discloses temporary TP53 inhibitors and their use for treatment of disease. International Patent Publication No. WO 2006/035434 and US Patent Publication No. 2008/0108583 both assigned to the assignee of the present application and hereby incorporated by reference in their entirety discloses TP53 inhibitors for the treatment of, inter alia, acute renal failure and hearing loss. International Patent Publication No. WO 2009/001359 assigned to the assignee of the present application, hereby incorporated by reference in its entirety, discloses siRNA inhibitors for TP53; HTRA2; KEAP1; SHC1-SHC, ZNHIT1, LGALS3, and HI95 (SESN2). U.S. Pat. No. 7,074,895 and US Publication No. US2006/0241290-A1 assigned to the assignee of the present application and hereby incorporated by reference in their entirety teach full length HI95 polypeptide and antibody. GB 2420119 discloses certain KEAP1 siRNA. International Patent Publication Nos. WO 03/087368 and WO 03/087367 teach methods of treating various eye and CNS diseases with RNAi to selected target genes.

There remains an unmet need for siRNA compounds having improved stability and/or improved activity and/or reduced off target effects and/or reduced immune response useful in therapeutic applications.

### SUMMARY OF THE INVENTION

The present application relates in part to chemically modified siRNA useful in inhibiting expression of target genes, having mRNA polynucleotide sequence set forth in any one of SEQ ID NOS 1-23 and listed in table A. The target genes include members of the NADPH oxidase (NOX) family of genes, TP53; HTRA2; KEAP1; SHC1, ZNHIT1, LGALS3, and HI95. The present application relates in part to chemically modified siRNA and in particular to chemically modified siRNA oligonucleotides having sense and complementary antisense sequences set forth in SEQ ID NOS:24-23,157. Oligonucleotide pairs useful in preparing the siRNA compounds are set forth in Tables A1-A18, B1-B15 and C1-C2, infra. The chemically modified siRNA compounds disclosed herein are useful in down regulating expression of one or more of the target genes. The compounds according to the present application exhibit properties that render them useful as therapeutic agents for treatment of a subject suffering from a disease, a disorder or an injury associated with target gene expression such as increased expression (up-regulation). Specifically the compounds exhibit high activity, and/or serum stability and/or reduced off-target effects and/or reduced adverse immune response as compared to an unmodified siRNA compound.

PCT Publication Nos. WO/2008/152636 and WO 2009/001359, assigned to the assignee of the present application and hereby incorporated by reference in their entirety, disclose some of the antisense and sense oligonucleotide pairs disclosed herein.

The present application also provides pharmaceutical compositions comprising one or more such oligonucleotides, and a pharmaceutically acceptable excipient.

The present application further relates to methods for treating or preventing the incidence or severity of various diseases, injuries or conditions in a subject in need thereof wherein the disease, injury or condition and/or symptoms associated therewith is selected from the group consisting of a neurodegenerative disease or disorder, an ophthalmic disease or condition, a respiratory disease, an ischemic disease or ischemia-reperfusion injury, an angiogenesis-related condition, a hearing impairment or any other disease, injury, condition or combination of conditions as disclosed herein. Such methods involve administering to a mammal in need of such treatment a prophylactically or therapeutically effective amount of one or more such chemically modified siRNA compound, which suppresses or reduces (down-regulates) expression and/or activity of one or more of the target genes, disclosed herein. Preferably the mammal is a human. The Nox family of genes refers to NOX4, NOX1, NOX2 (gp91phox, CYBB), NOX5, DUOX2, NOXO1, NOXO2 (NCF1), NOXA1 and NOXA2 (p67phox, NCF2), listed in Table 1 hereinbelow. Other target genes include TP53; HTRA2; KEAP1; SHC1; ZNHIT1; LGALS3 and HI95, listed in Table 1, hereinbelow.

The present application provides novel siRNA oligonucleotidepairs, set forth as antisense and sense pairs in Tables A1-A18 B1-B15 and C1-C2, SEQ ID NOS: 24-23,157. The siRNA oligonucleotides shown in any one of the tables consist of unmodified ribonucleotides or comprise a combination of unmodified ribonucleotides and modified ribonucleotides and/or unconventional moieties, and optionally a capping moiety.

Accordingly in one aspect the present application provides a siRNA compound having the following structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide which may be unmodified or modified, or an unconventional moiety;

wherein each of (N)x and (N′)y is an oligonucleotide in which each consecutive N or N′ is joined to the next N or N′ by a covalent bond;

wherein Z and Z′ may be present or absent, but if present is independently 1-5 consecutive nucleotides covalently attached at the 3′ terminus of the strand in which it is present;

wherein z″ may be present or absent, but if present is a capping moiety covalently attached at the 5′ terminus of (N′)y;

each of x and y is independently an integer between 18 and 40;

wherein the sequence of (N′)y has complementary to the sequence of (N)x; and wherein (N)x comprises an oligonucleotide set forth in any one of SEQ ID NOS: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In certain preferred embodiments (N)x comprises an antisense sequence set forth in any one of SEQ ID Nos:23012-23157.

In certain preferred embodiments (N)x comprises an antisense sequences set forth in any one of SEQ ID Nos: 668-1311, 1812-2311.

In certain preferred embodiments (N)x comprises an antisense sequences set forth in any one of SEQ ID Nos: 4050-4549 or 5571-6391.

In another aspect the present application provides a compound having the following structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide which may be unmodified or modified, or an unconventional moiety and wherein at least one of N or N′ is a modified ribonucleotide or an unconventional moiety;

wherein each of (N)x and (N′)y is an oligonucleotide in which each consecutive N or N′ is joined to the next N or N′ by a covalent bond;

wherein Z and Z′ may be present or absent, but if present is independently 1-5 consecutive nucleotides covalently attached at the 3′ terminus of the strand in which it is present;

wherein z″ may be present or absent, but if present is a capping moiety covalently attached at the 5′ terminus of (N′)y;

each of x and y is independently an integer between 18 and 40;

wherein the sequence of (N′)_{y} has complementary to (N)x; and wherein the sequence of (N)ₓ comprises an antisense sequence having complementarity to about 18 to about 40 consecutive ribonucleotides in an mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having substantial complementarity to about 18 to about 40 consecutive ribonucleotides in an mRNA set forth in any one of SEQ ID NOS:1-23.

In certain preferred embodiments (N)x comprises any one of the antisense sequences set forth in any one of SEQ ID Nos:23012-23157. The siRNA compounds according to the antisense and sense pairs as set forth in any one of Tables A1-A18, B1-B15 and C1-C2 comprise a combination of unmodified ribonucleotides and modified ribonucleotides and/or unconventional moieties, and optionally a capping moiety z″.

In some embodiments of the above structure (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide and the sense (N′)y comprises at least one mirror nucleotide, or a nucleotide joined to an adjacent nucleotide by a 2′-5′ internucleotide phosphate bond.

In additional embodiments (N)x comprises modified ribonucleotides in alternating positions wherein each N at the 5′ and 3′ termini are 2′OMe sugar modified ribonucleotides, and the middle ribonucleotide is not modified, e.g. ribonucleotide in position 10 in a 19-mer strand or position 12 in a 23-mer strand.

For all the structures, in some embodiments the covalent bond joining each consecutive N or N′ is a phosphodiester bond. In various embodiments all the covalent bonds are phosphodiester bonds.

In various embodiments x=y and each of x and y is 19, 20, 21, 22 or 23. In some embodiments x=y=23. In other embodiments x=y=19.

In one embodiment of the above structure, the compound comprises at least one mirror nucleotide at one or both termini in (N′)y. In various embodiments the compound comprises two consecutive mirror nucleotides, one at the 3′ penultimate position and one at the 3′ terminus in (N′)y. In one preferred embodiment x=y=19 and (N′)y comprises a mirror nucleotide at position 18.

In some embodiments the mirror nucleotide is selected from an L-ribonucleotide and an L-deoxyribonucleotide. In various embodiments the mirror nucleotide is an L-deoxyribonucleotide. In some embodiments y=19 and (N′)y, consists of unmodified ribonucleotides at positions 1-17 and 19 and one L-DNA at the 3′ penultimate position (position 18). In other embodiments y=19 and (N′)y consists of unmodified ribonucleotides at position 1-16 and 19 and two consecutive L-DNA at the 3′ penultimate position (positions 17 and 18). In one preferred embodiment x=y=19 and (N′)y comprises an L-deoxyribonucleotide at position 18.

In another embodiment of the above structure, (N′)y further comprises one or more nucleotides containing an intra-sugar bridge at one or both termini.

In another embodiment of the above structure, (N′)y comprises at least two consecutive nucleotide joined together to the next nucleotide by a 2′-5′ phosphodiester bond at one or both termini. In certain preferred embodiments in (N′)y the 3′ penultimate nucleotide is linked to the 3′ terminal nucleotide with a 2′-5′ phosphodiester bridge.

In certain preferred embodiments the compound of the application is a blunt-ended (z″, Z and Z′ are absent), double stranded oligonucleotide structure, x=y and x=19 or 23, wherein (N′)y comprises unmodified ribonucleotides in which three consecutive nucleotides at the 3′ terminus are joined together by two 2′-5′ phosphodiester bonds; and (N)x comprises alternating unmodified and 2′-O methyl sugar-modified ribonucleotides.

In some embodiments, neither (N)x nor (N′)y are phosphorylated at the 3′ and 5′ termini. In other embodiments either or both (N)x and (N′)y are phosphorylated at the 3′ termini.

In certain embodiments for all the above-mentioned structures, the compound is blunt ended, for example wherein both Z and Z′ are absent. In an alternative embodiment, the compound comprises at least one 3′ overhang and or a 5′ capping moiety at the 5′ terminus of (N′)y, wherein at least one of Z or Z′ or z″ is present. Z, Z′ and z″ are independently one or more covalently linked modified or non-modified nucleotides, for example inverted dT or dA; dT, LNA, mirror nucleotide and the like. In some embodiments each of Z and Z′ are independently selected from dT and dTdT. In other enmbodiments each of Z and Z′ is independently selected from 1-5 covalently attached nucleotide or non-nucleotide moieties.

In a second aspect the present application provides a pharmaceutical composition comprising one or more compounds of the present application, in an amount effective to down-regulate target gene expression, and a pharmaceutically acceptable carrier wherein the target gene is selected from NOX4, NOX1, NOX2 (gp91phox, CYBB), NOX5, DUOX2, NOXO1, NOXO2 (NCF1), NOXA1, NOXA2 (p67phox, NCF2), TP53; HTRA2; KEAP1; SHC1, ZNHIT1, LGALS3, and HI95 having mRNA set forth in any one of SEQ ID NOS 1-23.

In another aspect, the present application relates to a method for the treatment of a subject in need of treatment for a disease, injury or disorder or symptom or condition associated with the disease, injury or disorder, associated with the expression of a at least one target gene comprising administering to the subject an amount of at least one siRNA which reduces (down-regulates) or inhibits expression or over-expression of at least one target gene. In preferred embodiments the at least one siRNA compound is chemically modified according to the embodiments of the present application.

In some embodiments, the present application relates to a method for the treatment of a subject in need of treatment for a disease, injury or disorder or symptom or condition associated with the disease, injury or disorder, associated with the expression of at least two target genes comprising administering to the subject at least two siRNA compounds which reduce (down-regulate) or inhibit expression or over-expression of the target genes. In preferred embodiments the siRNA compounds are chemically modified according to the embodiments of the present application. In some embodiments, the siRNA compounds are administered by the same route, either from the same or from different pharmaceutical compositions. However, in other embodiments, using the same mute of administration for two or more of the therapeutic siRNA compounds either is impossible or is not preferred. Persons skilled in the art are aware of the best modes of administration for each therapeutic agent, either alone or in a combination

More specifically, the present application provides methods, compounds and compositions useful in therapy for treating a subject suffering from or at risk of a neurodegenerative disease (ND), injury or disorder including spinal cord injury, Alzheimer's Disease (AD) and Amyotrophic lateral sclerosis (ALS); acute renal failure (ARF); hearing loss; an ophthalmic disease including glaucoma, dry eye syndrome and ION; a respiratory disease including acute respiratory distress syndrome (ARDS), chronic obstructive pulmonary disease (COPD) and other acute lung and respiratory injuries; injury (e.g. ischemia-reperfusion injury), graft disfunction and acute rejection after organ transplantation, including lung, kidney, bone marrow, heart, pancreas, cornea or liver transplantation; nephrotoxicity; pressure sores, oral mucositis. The methods of the application comprise administering to the subject one or more siRNA compounds which down-regulate expression of a target gene in a therapeutically effective dose so as to thereby treat the patient.

An additional embodiment of the present application provides for the use of a therapeutically effective amount of a siRNA compound according to the application for the preparation of a medicament for promoting recovery in a patient suffering from a disorder listed herein.

### DETAILED DESCRIPTION OF THE INVENTION

The present application, relates to novel oligonucleotide compounds useful in inhibiting one or more of the target genes disclosed herein for the treatment of neurodegenerative diseases and disorders, CNS injury, eye diseases, respiratory disorders, microvascular disorders, hearing disorders and ischemic conditions, inter alia. As will be described herein, the preferred inhibitors to be used with the present application are chemically modified siRNA.

Compounds and compositions comprising same which inhibit one or more of the target genes are discussed herein at length, and any of said compounds and/or compositions may be beneficially employed in the treatment of a patient suffering from or at risk of a disease, an injury or a disorder associated with target gene expression or over-expression.

The present application relates generally to compounds which down-regulate expression of target genes, particularly to novel small interfering RNAs (siRNAs), and to the use of these siRNAs in the treatment of various diseases and medical conditions. Particular diseases and conditions to be treated are hearing loss including chemical-induced ototoxicity; acute renal failure (ARF), nephritis, ocular disease (e.g. glaucoma), Acute Respiratory Distress Syndrome (ARDS) and other acute lung injuries, lung transplantationkidney transplantation, nephrotoxicity, spinal cord injury, pressure sores, osteoarthritis (OA) and Chronic Obstructive Pulmonary Disease (COPD) and prevention of ischemia-reperfusion (I/R) injury in organ transplant patients, including lung and kidney transplant. In some embodiments one or more of the compounds of the application is useful in neuroprotection, including optic nerve protection.

Lists of preferred 19-mer siRNA pairs are provided in SEQ ID NOS:22,866-23,157. Other oligonucleotide pairs useful in preparation of a siRNA drug candidate are shown in Tables A1-A18, B1-B15 and C1-C2.

Methods, molecules and compositions, which inhibit the target genes are discussed herein at length, and any of said molecules and/or compositions may be beneficially employed in the treatment of a patient suffering from any of said conditions. Table 1, below, sets forth the gene identification number (gi) with an NCBI accession number for the respective mRNA sequences, corresponding oligomer tables' listings and associated indications in which inhibition of gene expression is useful.

TABLE 1

| | | Preferred indications or diseases to be |
|---|---|---|
| | | prevented or treated according to the |
| | Full name and Human Gene ID | present application (certain preferred |
| Gene | (mRNA) | indications are underlined) |
| NOX4 | NADPH oxidase 4 | ARF, hearing loss, ARDS, glaucoma, |
| | (gi:20149638, NM_016931, SEQ | spinal cord injury (SCI), COPD, diabetic |
| | ID NO:1) | retinopathy (DR), nephritis, I/R injury in |
| | | organ transplantation patients |
| NOX1 | NADPH oxidase 1 | ARF, hearing loss, ARDS, glaucoma, |
| | (gi:21614529, NM_007052 isoform | spinal cord injury, COPD, DR, nephritis, |
| | 1L; SEQ ID NO:2) | ND |
| | (gi:7669509, NM_013955 isoform | |
| | 1Lv; SEQ ID NO:3) | |
| NOX2 | NADPH oxidase 2 | ND (AD and ALS), ARF, hearing loss, |
| (gp91pho, | (gi:6996020, NM_000397; SEQ ID | ARDS, glaucoma, spinal cord injury, |
| CYBB) | NO:4) | COPD, OA, DR, nephritis, I/R injury in |
| | | organ transplantation patients |
| NOX5 | NADPH oxidase 5 | ARF, hearing loss, ARDS, glaucoma, |
| | (gi:20127623, NM_024505; SEQ | spinal cord injury, COPD, DR, nephritis, |
| | ID NO:5) | ND |
| DUOX2 | Dual oxidase 2 | ARF, hearing loss, ARDS, glaucoma, |
| | (gi:132566531, NM_014080; SEQ | spinal cord injury, COPD, DR, nephritis, |
| | ID NO:6) | ND |
| NOXO1 | NADPH oxidase organizer 1 | ARF, hearing loss, ARDS, glaucoma, |
| | (gi:34222190, variant a, | spinal cord injury, COPD, DR, nephritis, |
| | NM_144603, SEQ ID NO:7) | ND |
| | (gi:41281810, variant b, | |
| | NM_172167; SEQ ID NO:8) | |
| | (gi:41281827, variant c, | |
| | NM_172168; SEQ ID NO:9) | |
| NOXO2 | NADPH oxidase organizer 2 | ARF, hearing loss, ARDS, glaucoma, |
| (p47phox, | (gi:115298671, NM_000265; SEQ | spinal cord injury, COPD, DR, nephritis, |
| NCF1) | ID NO:10) | ND |
| NOXA1 | NADPH oxidase activator 1 | ARF, hearing loss, ARDS, glaucoma, |
| | (gi:41393186, NM_006647; SEQ | spinal cord injury, COPD, DR, nephritis |
| | ID NO:11) | |
| NOXA2 | NADPH oxidase activator 2 | ARF, hearing loss, ARDS, glaucoma, |
| (p67phox, | (gi:67189969, NM_000433; SEQ | spinal cord injury, COPD, DR, nephritis |
| NCF2) | ID NO:12) | |
| p53 (TP53) | tumor protein p53 | organ transplant (e.g. lung, kidney), |
| | gi8400737, NM_000546.2 | glaucoma, hearing loss, AKI, acute lung |
| | SEQ ID NO:13 | injury, ischemic optic neuropathy (ION) |
| HTRA2 | Htra serine peptidase 2 | ARF, hearing loss, ARDS, glaucoma, SCI, |
| | var 1 gi:73747817, NM_013247 | COPD, osteoarthritis, DR, toxicity, organ |
| | SEQ ID NO:14 | transplant, acute ischemia-reperfusion lung |
| | var 2 gi:73747818, NM_145074 | injury |
| | SEQ ID NO:15 | |
| KEAP1 | Kelch-like ECH-associated protein 1 | ARF, hearing loss, ARDS, glaucoma, SCI, |
| | var 1 gi:45269144 NM_203500 | COPD, osteoarthritis, diabetic retinopathy, |
| | SEQ ID NO:16 | pressure sores, nephrotoxicity, |
| | var 2 gi:45269143 NM_012289 | neurotoxicity, organ transplant |
| | SEQ ID NO:17 | |
| SHC1 | Src homology 2 domain containing) | ARF, hearing loss, ARDS, glaucoma, SCI, |
| | transforming prot. 1 | COPD, osteoarthritis, diabetic retinopathy, |
| | var 1 gi:52693920 NM_183001 | pressure sores, toxicity, organ transplant, |
| | SEQ ID NO:18 | acute ischemia-reperfusion lung injury, |
| | var 2 gi:34147725 NM_003029 | AD, ALS |
| | SEQ ID NO:19 | |
| ZNHIT1 | Zn finger HIT type 1 | ARF, hearing loss, ARDS, glaucoma, SCI, |
| | gi:375944391; NM_006349 | COPD, osteoarthritis, DR, pressure sores, |
| | SEQ ID NO:20 | toxicity, organ transplant |
| LGALS3 | lectin galactoside-binding soluble 3 | ARF, hearing loss, ARDS, glaucoma, SCI, |
| | var 1 gi:115430222 NM_002306 | COPD, osteoarthritis, DR, pressure sores, |
| | SEQ ID NO:21 | organ transplant |
| | var 2 gi:115430224 NR_003225 | |
| | SEQ ID NO:22 | |
| HI95 | Sestrin2 | ARF, hearing loss, ARDS, glaucoma, SCI, |
| | gi:32454742 NM_031459 | COPD, osteoarthritis, DR, pressure sores, |
| | SEQ ID NO:23 | organ transplant |

ND: neurodegenerative diseases and disorders;
AD: Alzheimer's disease;
ALS: Amyotrophic Lateral Sclerosis;
AKI: acute kidney injury;
ARF: acute renal failure,
ARDS, acute respiratory distress syndrome,
COPD chronic obstructive pulmonary disease,
DR: diabetic retinopathy,
ION: ischemic optic neuropathy;
I/R: ischemia-reperfusion injury;
SCI: spinal cord injury.

In various embodiments the siRNAs of the present application possess structures and modifications which increase activity and/or increase stability and/or minimize toxicity; the novel modifications of the siRNAs of the present application are beneficially applied to double stranded RNA useful in preventing or attenuating one or more of the target genes' expression.

Definitions

For convenience certain terms employed in the specification, examples and claims are described herein.

It is to be noted that, as used herein, the singular forms “a”, “an” and “the” include plural forms unless the content clearly dictates otherwise.

Where aspects or embodiments of the application are described in terms of Markush groups or other grouping of alternatives, those skilled in the art will recognize that the application is also thereby described in terms of any individual member or subgroup of members of the group.

A “siRNA inhibitor” is a compound which is capable of reducing the expression or over-expression of a target gene or the activity of the product of such gene to an extent sufficient to achieve a desired biological or physiological effect. The term “siRNA inhibitor” as used herein refers to one or more of a siRNA, shRNA, synthetic shRNA; miRNA. Inhibition may also be referred to as down-regulation or, for RNAi, silencing.

The term “inhibit” as used herein refers to reducing the expression or over-expression of a target gene or the activity of the product of such gene to an extent sufficient to achieve a desired biological or physiological effect. Inhibition may be complete or partial.

As used herein, the term “inhibition” of a target gene refers to inhibition (down-regulation) of the gene expression (transcription or translation) or of a polypeptide activity of a gene selected from the group NOX4, NOX1, NOX2 (gp91phox, CYBB), NOX5, DUOX2, NOXO1, NOXO2, NOXA1, NOXA2 (p67phox), TP53; HTRA2; KEAP1; SHC1, ZNHIT1, LGALS3, and HI95, or SNP (single nucleotide polymorphism) or other variants thereof. The gi number for the mRNA of each target gene is set forth in Table 1. The polynucleotide sequence of target mRNA sequence, refers to the mRNA sequences set forth in SEQ ID NO:1-23, or any homologous sequences thereof preferably having at least 70% identity, more preferably 80% identity, even more preferably 90% or 95% identity to any one of mRNA set forth in SEQ ID NOS:1-23. Therefore, polynucleotide sequences derived from any one of SEQ ID NO:1-23 which have undergone mutations, alterations or modifications as described herein are encompassed in the present application. The terms “mRNA polynucleotide sequence” and “mRNA” are used interchangeably.

A “NOX inhibitor” is a compound which is capable of down-regulating the activity of the NOX genes or NOX gene products, particularly one of the human NOX genes or gene products. Such inhibitors include substances that affect the transcription or translation of the gene as well as substances that affect the activity of the gene product. Examples of such inhibitors include, inter alia: polynucleotides such as antisense (AS) fragments, siRNA, or vectors comprising them; catalytic RNAs such as ribozymes. Specific siRNA inhibitors are provided herein.

As used herein, the terms “polynucleotide” and “nucleic acid” are used interchangeably and refer to nucleotide sequences comprising unmodified and or modified deoxyribonucleic acid (DNA), and ribonucleic acid (RNA). The terms should also be understood to include, as equivalents, analogs of either RNA or DNA made from nucleotide analogs. Throughout this application mRNA sequences are set forth as representing the corresponding genes.

“Oligonucleotide” or “oligomer” refers to a deoxyribonucleotide or ribonucleotide sequence from about 2 to about 50 nucleotides. Each DNA or RNA nucleotide is independently natural or synthetic, and/or modified or unmodified. Modifications include changes to the sugar moiety, the base moiety and or the linkages between nucleotides in the oligonucleotide. The compounds of the present application encompass molecules comprising deoxyribonucleotides, ribonucleotides, modified deoxyribonucleotides, modified ribonucleotides, unconventional moieties and combinations thereof

“Nucleotide” is meant to encompass deoxyribonucleotides and ribonucleotides, which is natural or synthetic, and/or modified or unmodified. Modifications include changes to the sugar moiety, the base moiety and or the linkages between ribonucleotides in the oligoribonucleotide. According to one aspect the present application provides inhibitory oligonucleotide compounds comprising unmodified and modified nucleotides and/or unconventional moieties. The compound comprises at least one modified nucleotide selected from the group consisting of a sugar modification, a base modification and an internucleotide linkage modification and may contain DNA, and modified nucleotides such as LNA (locked nucleic acid), ENA (ethylene-bridged nucleic acid, PNA (peptide nucleic acid), arabinoside, PACE, mirror nucleotide, or nucleotides with a 6 carbon sugar.

All analogs of, or modifications to, a nucleotide/oligonucleotide are employed with the present application, provided that said analog or modification does not substantially adversely affect the function of the nucleotide/oligonucleotide. Acceptable modifications include modifications of the sugar moiety, modifications of the base moiety, modifications in the internucleotide linkages and combinations thereof.

In one embodiment the compound comprises a 2′ modification on the sugar moiety of at least one ribonucleotide (“2′ sugar modification”). In certain embodiments the compound comprises 2′O-alkyl or 2′-fluoro or 2′O-allyl or any other 2′ modification, optionally on alternate positions. Other stabilizing modifications are also possible (e.g. terminal modifications). In some embodiments a preferred 2′O-alkyl is 2′O-methyl (methoxy) sugar modification.

In some embodiments the backbone of the oligonucleotides is modified and comprises phosphate-D-ribose entities. In other embodiments the backbone comprises thiophosphate-D-ribose entities, triester, thioate, 2′-5′ bridged backbone (also referred to as 5′-2′), PACE and the like.

As used herein, the terms “non-pairing nucleotide analog” means a nucleotide analog which comprises a non-base pairing moiety including but not limited to: 6 des amino adenosine (Nebularine), 4-Me-indole, 3-nitropyrrole, 5-nitroindole, Ds, Pa, N3-Me ribo U, N3-Me riboT, N3-Me dC, N3-Me-dT, N1-Me-dG, N1-Me-dA, N3-ethyl-dC, N3-Me dC. In some embodiments the non-base pairing nucleotide analog is a ribonucleotide. In other embodiments it is a deoxyribonucleotide.

Other modifications include terminal modifications on the 5′ and/or 3′ region of the oligonucleotides and are also known as capping moieties. Such terminal modifications are selected from a nucleotide, a modified nucleotide, a lipid, a peptide, and a sugar.

The term “amino acid” refers to a molecule which consists of any one of the 20 naturally occurring amino acids, amino acids which have been chemically modified (see below), or synthetic amino acids.

The term “polypeptide” refers to a molecule composed of two or more amino acids residues. The term includes peptides, polypeptides, proteins and peptidomimetics.

“Apoptosis” refers to a physiological type of cell death which results from activation of some cellular mechanisms, i.e. death that is controlled by the machinery of the cell. Apoptosis may, for example, be the result of activation of the cell machinery by an external trigger, e.g. a cytokine or anti-FAS antibody, which leads to cell death or by an internal signal. The term “programmed cell death” is also used interchangeably with “apoptosis”.

“Apoptosis-related disease” refers to a disease whose etiology is related either wholly or partially to the process of apoptosis. The disease may be caused either by a malfunction of the apoptotic process (such as in cancer or an autoimmune disease) or by over activity of the apoptotic process (such as in certain neurodegenerative diseases). For example, apoptosis is a significant mechanism in dry Age-Related Macular Degeneration (AMD), whereby slow atrophy of photoreceptor and pigment epithelium cells, primarily in the central (macular) region of retina takes place. Neuroretinal apoptosis is also a significant mechanism in diabetic retinopathy (DR).

An “expression vector” refers to a vector that has the ability to incorporate and express heterologous DNA fragments in a foreign cell. Many prokaryotic and eukaryotic expression vectors are known and/or commercially available. Selection of appropriate expression vectors is within the knowledge of those having skill in the art.

By “small interfering RNA” (siRNA) is meant an RNA molecule which down-regulates or silences (prevents) the expression of a gene/mRNA of its endogenous cellular counterpart. RNA interference (RNAi) refers to the process of sequence-specific post transcriptional gene silencing in mammals mediated by small interfering RNAs (siRNAs) (Fire et al, 1998, Nature 391, 806). The corresponding process in plants is commonly referred to as specific post transcriptional gene silencing or RNA silencing and is also referred to as quelling in fungi. The RNA interference response may feature an endonuclease complex containing an siRNA, commonly referred to as an RNA-induced silencing complex (RISC), which mediates cleavage of single-stranded RNA having sequence complementary to the antisense strand of the siRNA duplex. Cleavage of the target RNA may take place in the middle of the region complementary to the antisense strand of the siRNA duplex (Elbashir et al 2001, Genes Dev., 15, 188). For information on these terms and proposed mechanisms, see Bernstein E., et al., 2001 November; 7(11):1509-21; and Nishikura K.: Cell. 2001. 107(4):415-8. Examples of siRNA molecules which are used in the present application are provided in Tables A1-A18, B1-B15 and C1-C2.

siRNA and RNA Interference

During recent years, RNAi has emerged as one of the most efficient methods for inactivation of genes (Nature Reviews, 2002, v. 3, p. 737-47; Nature, 2002, v. 418, p. 244-51). As a method, it is based on the ability of dsRNA species to enter a specific protein complex, where it is then targeted to the complementary cellular RNA and specifically degrades it. In more detail, dsRNAs are digested into short (17-29 bp) inhibitory RNAs (siRNAs) by type III RNAses (DICER, DROSHA, etc) (Nature, 2001, v. 409, p. 363-6; Nature, 2003, 0.425, p. 415-9). The specific RISC protein complex recognizes these fragments and complementary mRNA. The whole process is culminated by endonuclease cleavage of target mRNA (Nature Reviews, 2002, v. 3, p. 737-47; Curr Opin Mol Ther. 2003 5(3):217-24).

PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164 and other relate to the phenomenon of RNAi.

RNA interference (RNAi) is based on the ability of dsRNA specie to enter a cytoplasmic protein complex, where it is then targeted to the complementary cellular RNA and specifically degrades it. The RNA interference response features an endonuclease complex containing a siRNA, commonly referred to as an RNA-induced silencing complex (RISC), which mediates cleavage of single-stranded mRNA having a sequence complementary to the antisense strand of the siRNA duplex. Cleavage of the target mRNA may take place in the middle of the region complementary to the antisense strand of the siRNA duplex (Elbashir et al., Genes Dev., 2001, 15(2):188-200). In more detail, longer dsRNAs are digested into short (17-29 bp) dsRNA fragments (also referred to as short inhibitory RNAs, “siRNAs”) by type III RNAses (DICER, DROSHA, etc.; Bernstein et al., Nature, 2001, 409(6818):363-6; Lee et al., Nature, 2003, 425(6956):415-9). The RISC protein complex recognizes these fragments and complementary mRNA. The whole process is culminated by endonuclease cleavage of target mRNA (McManus & Sharp, Nature Rev Genet, 2002, 3(10):737-47; Paddison & Hannon, Curr Opin Mol Ther. 2003, 5(3):217-24). (For additional information on these terms and proposed mechanisms, see for example Bernstein et al., RNA 2001, 7(11):1509-21; Nishikura, Cell 2001, 107(4):415-8 and PCT publication WO 01/36646).

Attempts to study RNAi and to manipulate mammalian cells experimentally were frustrated by an active, non-specific antiviral defense mechanism which was activated in response to long dsRNA molecules (Gil et al. Apoptosis, 2000. 5:107-114). Later it was discovered that synthetic duplexes of 21 nucleotide RNAs could mediate gene specific RNAi in mammalian cells, without the stimulation of the generic antiviral defense mechanisms (see for example Elbashir et al. Nature 2001, 411:494-498 and Caplen et al. PNAS USA 2001, 98:9742-9747). As a result, small interfering RNAs (siRNAs), which are short double-stranded RNAs, have become powerful tools in attempting to understand gene function. Thus, RNA interference (RNAi) refers to the process of sequence-specific post-transcriptional gene silencing in mammals mediated by small interfering RNAs (siRNAs) (Fire et al, Nature 1998. 391, 806) or microRNAs (miRNA; Ambros, Nature 2004 431:7006,350-55; and Bartel, Cell. 2004. 116(2):281-97).

A siRNA is a double-stranded RNA molecule which inhibits, either partially or fully, the expression of a gene/mRNA of its endogenous or cellular counterpart, or of an exogenous gene such as a viral nucleic acid. Several studies have revealed that siRNA therapeutics are effective in vivo in both mammals and in humans. Bitko et al., have shown that specific siRNA molecules directed against the respiratory syncytial virus (RSV) nucleocapsid N gene are effective in treating mice when administered intranasally (Bitko et al., Nat. Med. 2005, 11(1):50-55). For a review of the use of siRNA as therapeutics, see for example Batik (J. Mol. Med. 2005. 83: 764-773).

siRNA Structures

The selection and synthesis of siRNA corresponding to known genes has been widely reported; (see for example Ui-Tei et al., J Biomed Biotech. 2006; 2006: 65052; Chalk et al., BBRC. 2004, 319(1): 264-74; Sioud & Leirdal, Met. Mol Biol.; 2004, 252:457-69; Levenkova et al., Bioinform. 2004, 20(3):430-2; Ui-Tei et al., NAR. 2004, 32(3):936-48).

For examples of the use of, and production of, modified siRNA see, for example, Braasch et al., Biochem. 2003, 42(26):7967-75; Chiu et al., RNA, 2003, 9(9):1034-48; PCT publications WO 2004/015107 (atugen AG) and WO 02/44321 (Tuschl et al). U.S. Pat. Nos. 5,898,031 and 6,107,094, teach chemically modified oligomers. US Patent Publication Nos. 2005/0080246 and 2005/0042647 relate to oligomeric compounds having an alternating motif and dsRNA compounds having chemically modified internucleoside linkages, respectively.

Other modifications have been disclosed. The inclusion of a 5′-phosphate moiety was shown to enhance activity of siRNAs in Drosophila embryos (Boutla, et al., Curr. Biol. 2001, 11:1776-1780) and is required for siRNA function in human HeLa cells (Schwarz et al., Mol. Cell, 2002, 10:537-48). Amarzguioui et al., (NAR, 2003, 31(2):589-95) showed that siRNA activity depended on the positioning of the 2′-O-methyl modifications. Holen et al (NAR. 2003, 31(9):2401-07) report that an siRNA having small numbers of 2′-O-methyl modified nucleosides gave good activity compared to wild type but that the activity decreased as the numbers of 2′-O-methyl modified nucleosides was increased. Chiu and Rana (RNA. 2003, 9:1034-48) teach that incorporation of 2′-O-methyl modified nucleosides in the sense or antisense strand (fully modified strands) severely reduced siRNA activity relative to unmodified siRNA. The placement of a 2′-O-methyl group at the 5′-terminus on the antisense strand was reported to severely limit activity whereas placement at the 3′-terminus of the antisense and at both termini of the sense strand was tolerated (Czauderna et al., NAR. 2003, 31(11):2705-16; WO 2004/015107). The siRNA molecules of the present application offer an advantage in that they are non-toxic and may be formulated as pharmaceutical compositions for treatment of various diseases.

NADPH Oxidase

The NADPH oxidase (NOX) family of proteins in humans consists of at least thirteen unique gene products: NOX1, NOX2 (gp91phox, CYBB), NOX3, NOX4, NOX5, DUOX1 and DUOX2 and associated proteins p22phox (CYBA), NOXO1, NOXO2 (p47phox, NCF1) NOXA1, NOXA2 (p67phox, NCF2) and p40phox (NCF4) (hereinafter “NOX genes”). Each member of the NOX family has a specific tissue expression pattern. For example, NOX1 is highly expressed in colonic epithelium, NOX2 has a broad expression pattern while NOX4 has been detected primarily in renal tubular epithelium (Geiszt et al., PNAS USA 2000, 97:8010-8014) and proliferating vascular smooth muscle (Lassegue et al., Circ Res. 2001 88(9):888-94). Reactive oxygen species (ROS) generated in many tissues has been shown to originate from the activity of NOX enzymes and NOX gene expression has been associated with various pathological processes (comprehensive review in Bedard and Krause, Physiol. Rev. 2007. 87:245-313, hereby incorporated by reference in its entirety).

Chemically Modified siRNA

The present application provides chemically modified siRNA compounds that are active and/or stable and/or have reduced off target or immunostimulatory effects, compared to unmodified siRNA oligonucleotides.

The nucleotides used in synthesizing siRNA are selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-haloadenine, 8-aminoadenine, 8-thiol adenine, 8-thiolalkyl adenines, 8-hydroxyl adenine and other 8-substituted adenines, 8-haloguanines, 8-aminoguanine, 8-thiolguanine, 8-thioalkyl guanines, 8-hydroxyl guanine and other substituted guanines, other aza and deaza adenines, other aza and deaza guanines, 5-trifluoromethyl uracil, 5-trifluoromethylcytosine and 5-fluoro cytosine.

In addition, analogs of polynucleotides can be prepared wherein the structure of one or more nucleotide is fundamentally altered and better suited for a therapeutic agent or an experimental reagent. An example of a nucleotide analog is a peptide nucleic acid (PNA) wherein the deoxyribose (or ribose) phosphate backbone in DNA (or RNA) is replaced with a polyamide backbone which is similar to that found in peptides. PNA analogs have been shown to be resistant to enzymatic degradation and to have extended lives in vivo and in vitro. Mirror nucleotides (“L-nucleotides”) may also be employed.

Possible modifications to the sugar residue are manifold and include 2′-O alkyl, locked nucleic acid (LNA), glycol nucleic acid (GNA), threose nucleic acid (TNA), arabinoside, altritol (ANA) and other 6-membered sugars including morpholinos, and cyclohexinyls.

LNA nucleotides are disclosed in International Patent Publication Nos. WO 00/47599, WO 99/14226, and WO 98/39352. Examples of siRNA compounds comprising LNA nucleotides are disclosed in Elmen et al., (NAR 2005. 33(0:439-447) and in PCT Patent Publication No. WO 2004/083430.

Examples of PACE nucleotides and analogs are disclosed in U.S. Pat. Nos. 6,693,187 and 7,067,641 both herein incorporated by reference in their entirety.

In various embodiments, the compounds of the present application are synthesized using one or more inverted nucleotides, for example inverted thymidine or inverted adenine (for example see Takei, et al., 2002. JBC 277(26):23800-06.)

Certain structures include siRNA compounds having one or a plurality of 2′-5′ internucleotide linkages (bridges or backbone).

In the context of the present application, a “mirror” nucleotide also referred to as a Spiegelmer, is a nucleotide with reverse chirality to the naturally occurring or commonly employed nucleotide, i.e., a mirror image of the naturally occurring or commonly employed nucleotide. In various embodiments, the mirror nucleotide is a ribonucleotide (L-RNA) or a deoxyribonucleotide (L-DNA). deoxyriboabasic 5′-phosphate. Non-limiting examples of mirror nucleotide include L-DNA (L-deoxyriboadenosine-3′-phosphate (mirror dA); L-deoxyribocytidine-3′-phosphate (mirror dC); L-deoxyriboguanosine-3′-phosphate (mirror dG); L-deoxyribothymidine-3′-phosphate (mirror image dT)) and L-RNA (L-riboadenosine-3′-phosphate (mirror rA); L-ribocytidine-3′-phosphate (mirror rC); L-riboguanosine-3′-phosphate (mirror rG); L-ribouracil-3′-phosphate (mirror dU). In some embodiment the mirror nucleotide further comprises at least one sugar, base and or backbone modification. U.S. Pat. No. 6,602,858 discloses nucleic acid catalysts comprising at least one L-nucleotide substitution.

The term “unconventional moiety” as used herein refers to abasic ribose moiety, an abasic deoxyribose moiety, a deoxyribonucleotide, a modified deoxyribonucleotide, a mirror nucleotide, a non-base pairing nucleotide analog and a nucleotide joined to an adjacent nucleotide by a 2′-5′ internucleotide phosphate bond; bridged nucleic acids including LNA and ethylene bridged nucleic acids.

The term “capping moiety” as used herein includes abasic ribose moiety, abasic deoxyribose moiety, modifications abasic ribose and abasic deoxyribose moieties including 2′ O alkyl modifications; inverted abasic ribose and abasic deoxyribose moieties and modifications thereof; C6-imino-Pi; a mirror nucleotide including L-DNA and L-RNA; 5′OMe nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5 -anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non bridging methylphosphonate and 5′-mercapto moieties.

Abasic deoxyribose moiety includes for example abasic deoxyribose-3′-phosphate; 1,2-dideoxy-D-ribofuranose-3-phosphate;1,4-anhydro-2-deoxy-D-ribitol-3-phosphate. Inverted abasic deoxyribose moiety includes inverted deoxyriboabasic; 3′,5′ inverted.

In some embodiments, backbone modifications, such as ethyl (resulting in a phospho-ethyl triester); propyl (resulting in a phospho-propyl triester); and butyl (resulting in a phospho-butyl triester) are also present. Other backbone modifications include polymer backbones, cyclic backbones, acyclic backbones, thiophosphate-D-ribose backbones, amidates, phosphonoacetates. Certain structures include siRNA compounds having one or a plurality of 2′-5′ internucleotide linkages (bridges or backbone). Other possible backbone modifications include thioate modifications or 2′-5′ bridged backbone modifications.

Additional modifications which present in various embodiments of the siRNA molecules according to the present application include nucleoside modifications such as artificial nucleic acids, peptide nucleic acid (PNA), morpholino and locked nucleic acid (LNA), glycol nucleic acid (GNA), threose nucleic acid (TNA), arabinoside, and mirror nucleoside (for example, beta-L-deoxynucleoside instead of beta-D-deoxynucleoside). In further embodiments, said molecules additionally contain modifications on the sugar, such as 2′ alkyl, 2′ fluoro, 2′O allyl 2′amine and 2′alkoxy. Many additional sugar modifications are discussed herein.

In further embodiments, the inhibitory nucleic acid molecules of the present application comprise one or more gaps and/or one or more nicks and/or one ore more mismatches. Without wishing to be bound by theory, gaps, nicks and mismatches have the advantage of partially destabilizing the nucleic acid/siRNA, so that it is more easily processed by endogenous cellular machinery such as DICER, DROSHA or RISC into its inhibitory components.

In the context of the present application, a gap in a nucleic acid refers to the absence of one or more internal nucleotides in one strand, while a nick in a nucleic acid refers to the absence of a internucleotide linkage between two adjacent nucleotides in one strand. In various embodiments, the siRNA molecules of the present application contain one or more gaps and/or one or more nicks.

Further provided by the present application is an siRNA compound encoded by any of the molecules disclosed herein, a vector encoding any of the molecules disclosed herein, and a pharmaceutical composition comprising any of the molecules disclosed herein or the vectors encoding them; and a pharmaceutically acceptable carrier.

The selection and synthesis of siRNA corresponding to known genes has been widely reported; see for example Ui-Tei et al., J Biomed Biotechnol. 2006; 65052; Chalk et al., BBRC. 2004, 319(1):264-74; Sioud & Leirdal, Met. Mol Biol. 2004, 252:457-69; Levenkova et al., Bioinform. 2004, 20(3):430-2; Ui-Tei et al., NAR. 2004, 32(3):936-48. For examples of the use and production of modified siRNA see for example Braasch et al., Biochem. 2003, 42(26):7967-75; Chiu et al., RNA. 2003, 9(9):1034-48; PCT Publication Nos. WO 2004/015107 and WO 02/44321 and U.S. Pat. Nos. 5,898,031 and 6,107,094.

Tables A1-A18 and B1-B15 provide sense and antisense oligonucleotide pairs useful in preparing corresponding siRNA compounds of the present application.

In general, some deviation from the target mRNA sequence is tolerated without compromising the siRNA activity (see e.g. Czauderna et al., 2003, NAR 31(11), 2705-2716). An siRNA of the application down-regulates target gene expression or over-expression on a post-transcriptional level with or without destroying the mRNA. Without being bound by theory, siRNA targets the mRNA for specific cleavage and degradation and/or inhibits translation from the targeted message.

Possible modifications on the 2′ moiety of the sugar residue include amino, fluoro, methoxy alkoxy, alkyl, amino, fluoro, chloro, bromo, CN, CF, imidazole, carboxylate, thioate, C₁ to C₁₀ lower alkyl, substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterozycloalkyl; heterozycloalkaryl; aminoalkylamino; polyalkylamino or substituted silyl, as, among others, described in European patents EP 0 586 520 B1 or EP 0 618 925 B1. One or more deoxyribonucleotides are also tolerated in the compounds of the present application. As used herein, in the description of any strategy for the design of molecules, RNAi or any embodiment of RNAi disclosed herein, the term “end modification” means a chemical entity added to the terminal 5′ or 3′ nucleotide of the sense and/or antisense strand. Examples for such end modifications include, but are not limited to, 3′ or 5′ phosphate, inverted abasic, abasic, amino, fluoro, chloro, bromo, CN, CF₃, methoxy, imidazolyl, carboxylate, phosphorothioate, C₁ to C₂₂ and lower alkyl, lipids, sugars and polyaminoacids (i.e. peptides), substituted lower alkyl, alkaryl or aralkyl, OCF₃, OCN, O-, S-, or N-alkyl; O-, S-, or N-alkenyl; SOCH₃; SO₂CH₃; ONO₂; NO₂, N₃; heterocycloalkyl; heterocycloalkaryl; aminoalkylamino; polyalkylamino or substituted silyl, as, among others, described in European patents EP 0586520B1 or EP 0618925 B1.

In some embodiments the siRNA is blunt ended, i.e. Z and Z′ are absent, on one or both ends. More specifically, in some embodiments, the siRNA is blunt ended on the end defined by the 5′-terminus of the first strand and the 3′-terminus of the second strand, and/or the end defined by the 3′-terminus of the first strand and the 5′-terminus of the second strand.

In other embodiments at least one of the two strands have an overhang of at least one nucleotide at the 5′-terminus; in some embodiments the overhang consist of at least one deoxyribonucleotide. In other embodiments at least one of the strands also optionally has an overhang of at least one nucleotide at the 3′-terminus. In various embodiments the overhang consists of from about 1 to about 5 nucleotides.

In some embodiments the compounds of the present application further comprise an end (terminus) modification. In some embodiments a biotin group is attached to either the most 5′ or the most 3′ nucleotide of the antisense and/or sense strand or to both ends. In one preferred embodiment the biotin group is coupled to a polypeptide or a protein, as among others described in Chun-Fang Xia et al., Mol. Pharmaceutics, 2009, 6 (3), pp 747-751. It is also within the scope of the present application that the polypeptide or protein is attached through any of the other aforementioned modifications.

The various end modifications as disclosed herein are preferably located at the ribose moiety of a nucleotide of the nucleic acid according to the present application. More particularly, in various embodiments the end modification is attached to or replaces any of the OH-groups of the ribose moiety, including but not limited to the 2′OH, 3′OH and 5′OH position, provided that the nucleotide thus modified is a terminal nucleotide. Inverted abasic or abasic are nucleotides, either deoxyribonucleotides or ribonucleotides which do not have a nucleobase moiety. This kind of compound is, inter alia, described in Sternberger, et al., (Antisense Nucleic Acid Drug Dev, 2002.12, 131-43).

The length of RNA duplex is from about 18 to about 40 ribonucleotides, preferably 19, 21 or 23 ribonucleotides. Further, in various embodiments the length of each strand independently has a length selected from the group consisting of about 15 to about 40 bases, preferably 18 to 23 bases and more preferably 19, 21 or 23 ribonucleotides.

In certain embodiments the complementarity between said antisense strand and the target nucleic acid is perfect. In some embodiments, the strands are substantially complementary, i.e. having one, two or up to three mismatches between said antisense strand and the target nucleic acid. “Substantially complementary” refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly “substantially identical” refers to identity of greater than about 84%, to another sequence.

In some embodiments the antisense strand and the sense strand are linked by a loop structure, which is comprised of a non-nucleic acid polymer such as, inter alia, polyethylene glycol. Alternatively, the loop structure is comprised of a nucleic acid, including modified and non-modified ribonucleotides and modified and non-modified deoxyribonucleotides.

Further, in some embodiments the 5′-terminus of the antisense strand of the siRNA is linked to the 3′-terminus of the sense strand, or the 3′-terminus of the antisense strand is linked to the 5′-terminus of the sense strand, said linkage being via a nucleic acid linker typically having a length between 2-100 nucleobases, preferably about 2 to about 30 nucleobases.

In preferred embodiments the compounds of the application have alternating ribonucleotides modified in at least one of the antisense and the sense strands of the compound, for 19 mer and 23 mer oligomers the ribonucleotides at the 5′ and 3′ termini of the antisense strand are modified in their sugar residues, and the ribonucleotides at the 5′ and 3′ termini of the sense strand are unmodified in their sugar residues. For 21 mer oligomers the ribonucleotides at the 5′ and 3′ termini of the sense strand are modified in their sugar residues, and the ribonucleotides at the 5′ and 3′ termini of the antisense strand are unmodified in their sugar residues, or have an optional additional modification at the 3′ terminus. As mentioned above, it is preferred that the middle nucleotide of the antisense strand is unmodified.

Additionally, the application provides siRNA compounds comprising a double stranded nucleic acid molecule wherein 1, 2, or 3 of the nucleotides in one strand or both strands are substituted thereby providing at least one base pair mismatch. The substituted nucleotides in each strand are preferably in the terminal region of one strand or both strands.

According to one preferred embodiment , the antisense and the sense strands of the oligonucleotide/siRNA are phosphorylated only at the 3′-terminus and not at the 5′-terminus. According to another preferred embodiment of the application, the antisense and the sense strands are non-phosphorylated. According to yet another preferred embodiment of the application, the 5′ most ribonucleotide in the sense strand is modified to abolish any possibility of in vivo 5′-phosphorylation.

In various embodiments siRNA sequence disclosed herein are modified to yield modifications/structures disclosed herein. The combination of sequence plus structure is novel. The novel siRNA compounds are useful in prevention and/or treatment of various medical conditions and pathologies, including diseases, injuries and disorders disclosed herein.

Particular molecules to be administered according to the methods of the present application are disclosed below under the heading “structural motifs”. For the sake of clarity, any of these molecules can be administered according to any of the methods of the present application.

Structural Motifs

According to the present application the siRNA compounds are chemically and or structurally modified according to one of the following modifications set forth in Structures below or as tandem siRNA or RNAstar.

In one aspect the present invention provides a compound having Structure (IX) set forth below:

| | | |
|---|---|---|
| (IX) | 5′ (N)x-Z 3′ | (antisense strand) |
| | 3′ Z′-(N′)y-z″ 5′ | (sense strand) |

wherein each of N and N′ is a ribonucleotide which may be unmodified or modified, or an unconventional moiety;

wherein each of (N)x and (N′)y is an oligonucleotide in which each consecutive N or N′ is joined to the next N or N′ by a covalent bond;

wherein Z and Z′ may be present or absent, but if present is independently 1-5 consecutive nucleotides covalently attached at the 3′ terminus of the strand in which it is present;

wherein z″ may be present or absent, but if present is a capping moiety covalently attached at the 5′ terminus of (N′)y;

wherein x=18 to 27;

wherein y=18 to 27;

wherein (N)x comprises modified and unmodified ribonucleotides, each modified ribonucleotide having a 2′-O-methyl on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide;

wherein in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond; and wherein the sequence of (N)x has complementary to the sequence of (N′)y; and the sequence of (N′)y has identity to the sequence of an mRNA encoded by a target gene.

In some embodiments x=y=19. In other embodiments x=y=23. In some embodiments x=y=19 and the at least one unconventional moiety is present at positions 15, 16, 17, or 18 in (N′)y. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18.

In other embodiments the unconventional moiety is an abasic moiety.

In some embodiments of Structure (IX) (N)x comprises nine alternating modified ribonucleotides. In other embodiments of Structure (IX) (N)x comprises nine alternating modified ribonucleotides further comprising a 2′O modified nucleotide at position 2. In some embodiments (N)x comprises 2′OMe sugar modified ribonucleotides at the odd numbered positions 1, 3, 5, 7, 9, 11, 13, 15, 17, 19. In other embodiments (N)x further comprises a 2′OMe sugar modified ribonucleotide at one or both of positions 2 and 18. In yet other embodiments (N)x comprises 2′OMe sugar modified ribonucleotides at positions 2, 4, 6, 8, 11, 13, 15, 17, 19.

In various embodiments z″ is present and is selected from an abasic ribose moiety, a deoxyribose moiety; an inverted abasic ribose moiety, a deoxyribose moiety; C6-amino-Pi; a mirror nucleotide.

In various embodiments of structure IX, Z and Z′ are absent; x=y=19; in (N′)y the nucleotide in at least one of positions 15, 16, 17, 18 and 19 comprises a nucleotide selected from an abasic pseudo-nucleotide, a mirror nucleotide, a deoxyribonucleotide and a nucleotide joined to an adjacent nucleotide by a 2′-5′ internucleotide bond; and

(N)x comprises alternating 2′OMe sugar modified ribonucleotides and unmodified ribonucleotides and the ribonucleotide located at the middle position of (N)x being modified or unmodified, preferably unmodified.

In various embodiments (N)x comprises 2′O-Me modified ribonucleotides at the odd numbered positions (5′ to 3′; positions 1, 3, 5, 7, 9, 11, 13, 15, 17, 19). In some embodiments (N)x further comprises 2′O-Me modified ribonucleotides at one or both positions 2 and 18. In other embodiments (N)x comprises 2′OMe sugar modified ribonucleotides at positions 2, 4, 6, 8, 11, 13, 15, 17, 19.

In various embodiments of Structure (IX) (N)ₓ comprises an antisense sequence set forth in any one of SEQ ID NOS: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In one aspect the present application provides a compound set forth as Structure (A):

| | | |
|---|---|---|
| (A) | 5′ (N)ₓ-Z 3′ | (antisense strand) |
| | 3′ Z′-(N′)_{y} 5′ | (sense strand) |

wherein each of N and N′ is a nucleotide selected from an unmodified ribonucleotide, a modified ribonucleotide, an unmodified deoxyribonucleotide and, a modified deoxyribonucleotide;

wherein each of (N)ₓ and (N′)_{y} is an oligonucleotide in which each consecutive N or N′ is joined to the next N or N′ by a covalent bond;

wherein each of x and y is an integer between 18 and 40;

wherein each of Z and Z′ may be present or absent, but if present is 1-5 consecutive nucleotides covalently attached at the 3′ terminus of the strand in which it is present;

wherein the sequence of (N′)_{y} has complementary to (N)x; and wherein the sequence of (N)ₓ comprises an antisense sequence set forth in any one of SEQ ID NOS: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In certain embodiments the present application provides a compound having Structure (B):

| | | |
|---|---|---|
| (B) | 5′ (N)x 3′ | antisense strand |
| | 3′ (N′)y 5′ | sense strand |

wherein each of (N)ₓ and (N′)_{y} is an oligomer in which each consecutive N or N′ is an unmodified ribonucleotide or a modified ribonucleotide joined to the next N or N′ by a covalent bond;

wherein each of x and y=19, 21 or 23 and (N)ₓ and (N′)_{y} are fully complementary wherein alternating ribonucleotides in each of (N)ₓ and (N′)_{y} are 2′OMe sugar modified ribonucleotides;

wherein the sequence of (N)_{y} has complementary to (N)x; and wherein the sequence of (N)ₓ comprises an antisense sequence set forth in any one of SEQ ID NOS: 1812-2311, 4050-4549, 6892-7391, 8532-9031, 10532-11031, 11177-11217, 11584-11757, 11862-11889, 13442-13941, 15224-15723, 16514-16941, 17881-18369, 18973-19291, 19906-20195, 20847-21177, 22366-22865, 23012-23157.

In some embodiments each of (N)ₓ and (N′)_{y} is independently phosphorylated or non-phosphorylated at the 3′ and 5′ termini.

In certain embodiments wherein each of x and y=19 or 23, each N at the 5′ and 3′ termini of (N)ₓ is modified; and each N′ at the 5′ and 3′ termini of (N′)_{y} is unmodified.

In certain embodiments wherein each of x and y=21, each N at the 5′ and 3′ termini of (N)ₓ is unmodified; and each N′ at the 5′ and 3′ termini of (N′)_{y} is modified.

In particular embodiments, when x and y=19, the siRNA is modified such that a 2′-O-methyl (2′-OMe) group is present on the first, third, fifth, seventh, ninth, eleventh, thirteenth, fifteenth, seventeenth and nineteenth nucleotide of the antisense strand (N)ₓ, and whereby the very same modification, i. e. a 2′-OMe group, is present at the second, fourth, sixth, eighth, tenth, twelfth, fourteenth, sixteenth and eighteenth nucleotide of the sense strand (N′)_{y}. In various embodiments these particular siRNA compounds are blunt ended at both termini.

In some embodiments, the present application provides a compound having Structure (C):

| | | |
|---|---|---|
| (C) | 5′ (N)x-Z 3′ | antisense strand |
| | 3′ Z′-(N′)y 5′ | sense strand |

wherein each of N and N′ is a nucleotide independently selected from an unmodified ribonucleotide, a modified ribonucleotide, an unmodified deoxyribonucleotide and a modified deoxyribonucleotide;

wherein each of (N)x and (N′)y is an oligomer in which each consecutive nucleotide is joined to the next nucleotide by a covalent bond;

each of x and y is an integer between 18 and 40;

wherein in (N)x the nucleotides are unmodified or (N)x comprises alternating 2′OMe sugar modified ribonucleotides and unmodified ribonucleotides; and the ribonucleotide located at the middle position of (N)x being modified or unmodified preferably unmodified;

wherein (N′)y comprises unmodified ribonucleotides further comprising one modified nucleotide at a terminal or penultimate position, wherein the modified nucleotide is selected from the group consisting of a mirror nucleotide, a bicyclic nucleotide, a 2′-sugar modified nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein if more than one nucleotide is modified in (N′)y, the modified nucleotides may be consecutive;

wherein each of Z and Z′ may be present or absent, but if present is 1-5 deoxyribonucleotides covalently attached at the 3′ terminus of any oligomer to which it is attached;

wherein the sequence of (N′)_{y} comprises a sequence having complementarity to (N)x; and wherein the sequence of (N)ₓ comprises an antisense sequence having complementary to about 18 to about 40 consecutive ribonucleotides in an mRNA set forth in any one SEQ ID NOS:1-23. In some preferred embodiments (N)ₓ comprises an antisense sequence set forth in any one of SEQ ID NOS: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In particular embodiments, x=y=19 and in (N)x each modified ribonucleotide is modified so as to have a 2′-O-methyl on its sugar and the ribonucleotide located at the middle of (N)x is unmodified. Accordingly, in a compound wherein x=19, (N)x comprises 2′-O-methyl sugar modified ribonucleotides at positions 1, 3, 5, 7, 9, 11, 13, 15, 17 and 19. In other embodiments, (N)x comprises 2′OMe sugar modified ribonucleotides at positions 2, 4, 6, 8, 11, 13, 15, 17 and 19. In other embodiments, (N)x comprises 2′OMe sugar modified ribonucleotides at positions 2, 4, 8, 11, 13, 15, 17 and 19 and may further comprise at least one abasic or inverted abasic unconventional moiety for example in position 6. In other embodiments, (N)x comprises 2′OMe modified ribonucleotides at positions 2, 4, 6, 8, 11, 13, 17 and 19 and may further comprise at least one abasic or inverted abasic unconventional moiety for example in position 15. In other embodiments, (N)x comprises 2′OMe modified ribonucleotides at positions 2, 4, 6, 8, 11, 13, 15, 17 and 19 and may further comprise at least one abasic or inverted abasic unconventional moiety for example in position 14. In other embodiments, (N)x comprises 2′OMe modified ribonucleotides at positions 1, 2, 3, 7, 9, 11, 13, 15, 17 and 19 and may further comprise at least one abasic or inverted abasic unconventional moiety for example in position 5. In other embodiments, (N)x comprises 2′OMe modified ribonucleotides at positions 1, 2, 3, 5, 7, 9, 11, 13, 15, 17 and 19 and may further comprise at least one abasic or inverted abasic unconventional moiety for example in position 6. In other embodiments, (N)x comprises 2′OMe modified ribonucleotides at positions 1, 2, 3, 5, 7, 9, 11, 13, 17 and 19 and may further comprise at least one abasic or inverted abasic unconventional moiety for example in position 15. In other embodiments, (N)x comprises 2′OMe modified ribonucleotides at positions 1, 2, 3, 5, 7, 9, 11, 13, 15, 17 and 19 and may further comprise at least one abasic or inverted abasic unconventional moiety for example in position 14. In other embodiments, (N)x comprises 2′OMe sugar modified ribonucleotides at positions 2, 4, 6, 7, 9, 11, 13, 15, 17 and 19 and may further comprise at least one abasic or inverted abasic unconventional moiety for example in position 5. In other embodiments, (N)x comprises 2′OMe sugar modified ribonucleotides at positions 1, 2, 4, 6, 7, 9, 11, 13, 15, 17 and 19 and may further comprise at least one abasic or inverted abasic unconventional moiety for example in position 5. In other embodiments, (N)x comprises 2′OMe sugar modified ribonucleotides at positions 2, 4, 6, 8, 11, 13, 14, 16, 17 and 19 and may further comprise at least one abasic or inverted abasic unconventional moiety for example in position 15. In other embodiments, (N)x comprises 2′OMe sugar modified ribonucleotides at positions 1, 2, 3, 5, 7, 9, 11, 13, 14, 16, 17 and 19 and may further comprise at least one abasic or inverted abasic unconventional moiety for example in position 15. In other embodiments, (N)x comprises 2′OMe sugar modified ribonucleotides at positions 2, 4, 6, 8, 11, 13, 15, 17 and 19 and may further comprise at least one abasic or inverted abasic unconventional moiety for example in position 7. In other embodiments, (N)x comprises 2′OMe sugar modified ribonucleotides at positions 2, 4, 6, 11, 13, 15, 17 and 19 and may further comprise at least one abasic or inverted abasic unconventional moiety for example in position 8. In other embodiments, (N)x comprises 2′OMe sugar modified ribonucleotides at positions 2, 4, 6, 8, 11, 13, 15, 17 and 19 and may further comprise at least one abasic or inverted abasic unconventional moiety for example in position 9. In other embodiments, (N)x comprises 2′OMe sugar modified ribonucleotides at positions 2, 4, 6, 8, 11, 13, 15, 17 and 19 and may further comprise at least one abasic or inverted abasic unconventional moiety for example in position 10. In other embodiments, (N)x comprises 2′OMe sugar modified ribonucleotides at positions 2, 4, 6, 8, 13, 15, 17 and 19 and may further comprise at least one abasic or inverted abasic unconventional moiety for example in position 11. In other embodiments, (N)x comprises 2′OMe sugar modified ribonucleotides at positions 2, 4, 6, 8, 11, 13, 15, 17 and 19 and may further comprise at least one abasic or inverted abasic unconventional moiety for example in position 12. In other embodiments, (N)x comprises 2′OMe sugar modified ribonucleotides at positions 2, 4, 6, 8, 11, 15, 17 and 19 and may further comprise at least one abasic or inverted abasic unconventional moiety for example in position 13.

In one embodiment of Structure (C), at least two nucleotides at either or both the 5′ and 3′ termini of (N′)y are joined by a 2′-5′ phosphodiester bond. In certain preferred embodiments x=y=19 or x=y=23; in (N)x the nucleotides alternate between 2′OMe sugar modified ribonucleotides and unmodified ribonucleotides, and the ribonucleotide located at the middle of (N)x being unmodified; and three nucleotides at the 3′ terminus of (N′)y are joined by two 2′-5′ phosphodiester bonds (set forth herein as Structure I).

In other preferred embodiments, x=y=19; in (N)x the nucleotides alternate between 2′OMe sugar modified ribonucleotides and unmodified ribonucleotides, and the ribonucleotide located at the middle of (N)x being unmodified; and four consecutive nucleotides at the 5′ terminus of (N′)y are joined by three 2′-5′ phosphodiester bonds. In a further embodiment, an additional nucleotide located in the middle position of (N)y may be 2′OMe sugar modified. In another preferred embodiment, in (N)x the nucleotides alternate between 2′OMe sugar modified ribonucleotides and unmodified ribonucleotides, and in (N′)y four consecutive nucleotides at the 5′ terminus are joined by three 2′-5′ phosphodiester bonds and the 5′ terminal nucleotide or two or three consecutive nucleotides at the 5′ terminus comprise 3′-O-methyl modifications.

In certain preferred embodiments of Structure (C), x=y=19 and in (N′)y, at least one position comprises an abasic or inverted abasic unconventional moiety, preferably five positions comprises an abasic or inverted abasic unconventional moieties. In various embodiments, the following positions comprise an abasic or inverted abasic: positions 1 and 16-19, positions 15-19, positions 1-2 and 17-19, positions 1-3 and 18-19, positions 1-4 and 19 and positions 1-5. (N′)y may further comprise at least one LNA nucleotide.

In certain preferred embodiments of Structure (C), x=y=19 and in (N′)y the nucleotide in at least one position comprises a mirror nucleotide, a deoxyribonucleotide and a nucleotide joined to an adjacent nucleotide by a 2′-5′ internucleotide bond.

In certain preferred embodiments of Structure (C), x=y=19 and (N′)y comprises a mirror nucleotide. In various embodiments the mirror nucleotide is an L-DNA nucleotide. In certain embodiments the L-DNA is L-deoxyribocytidine. In some embodiments (N′)y comprises L-DNA at position 18. In other embodiments (N′)y comprises L-DNA at positions 17 and 18. In certain embodiments (N′)y comprises L-DNA substitutions at positions 2 and at one or both of positions 17 and 18. In certain embodiments (N′)y further comprises a 5′ terminal cap nucleotide such as 5′-O-methyl DNA or an abasic or inverted abasic moiety as an overhang.

In yet other embodiments (N′)y comprises a DNA at position 15 and L-DNA at one or both of positions 17 and 18. In that structure, position 2 may further comprise an L-DNA or an abasic unconventional moiety.

Other embodiments of Structure (C) are envisaged wherein x=y=21 or wherein x=y=23; in these embodiments the modifications for (N′)y discussed above instead of being on positions 15, 16, 17, 18 are on positions 17, 18, 19, 20 for 21 mer and on positions 19, 20, 21, 22 for 23 mer; similarly the modifications at one or both of positions 17 and 18 are on one or both of positions 19 or 20 for the 21 mer and one or both of positions 21 and 22 for the 23 mer. All modifications in the 19 mer are similarly adjusted for the 21 and 23 mers.

According to various embodiments of Structure (C), in (N′)y 2, 3, 4, 5, 6, 7, 8 , 9, 10, 11, 12, 13 or 14 consecutive ribonucleotides at the 3′ terminus are linked by 2′-5′ internucleotide linkages In one preferred embodiment, four consecutive nucleotides at the 3′ terminus of (N′)y are joined by three 2′-5′ phosphodiester bonds, wherein one or more of the 2′-5′ nucleotides which form the 2′-5′ phosphodiester bonds further comprises a 3′-O-methyl sugar modification. Preferably the 3′ terminal nucleotide of (N′)y comprises a 2′OMe sugar modification. In certain preferred embodiments of Structure (C), x=y=19 and in (N′)y two or more consecutive nucleotides at positions 15, 16, 17, 18 and 19 comprise a nucleotide joined to an adjacent nucleotide by a 2′-5′ internucleotide bond. In various embodiments the nucleotide forming the 2′-5′ internucleotide bond comprises a 3′ deoxyribose nucleotide or a 3′ methoxy nucleotide. In some embodiments the nucleotides at positions 17 and 18 in (N′)y are joined by a 2′-5′ internucleotide bond. In other embodiments the nucleotides at positions 16, 17, 18, 16-17, 17-18, or 16-18 in (N′)y are joined by a 2′-5′ internucleotide bond.

In certain embodiments (N′)y comprises an L-DNA at position 2 and 2′-5′ internucleotide bonds at positions 16-17, 17-18, or 16-18. In certain embodiments (N′)y comprises 2′-5′ internucleotide bonds at positions 16-17, 17-18, or 16-18 and a 5′ terminal cap nucleotide.

According to various embodiments of Structure (C), in (N′)y 2, 3, 4, 5, 6, 7, 8, 9, 10, 11, 12, 13 or 14 consecutive nucleotides at either terminus or 2-8 modified nucleotides at each of the 5′ and 3′ termini are independently mirror nucleotides. In some embodiments the mirror nucleotide is an L-ribonucleotide. In other embodiments the mirror nucleotide is an L-deoxyribonucleotide. The mirror nucleotide may further be modified at the sugar or base moiety or in an internucleotide linkage.

In one preferred embodiment of Structure (C), the 3′ terminal nucleotide or two or three consecutive nucleotides at the 3′ terminus of (N′)y are L-deoxyribonucleotides.

In other embodiments of Structure (C), in (N′)y 2, 3, 4, 5, 6, 7, 8, 9, 10, 11, 12, 13 or 14 consecutive ribonucleotides at either terminus or 2-8 modified nucleotides at each of the 5′ and 3′ termini are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe). In one series of preferred embodiments, three, four or five consecutive nucleotides at the 5′ terminus of (N′)y comprise the 2′-OMe modification. In another preferred embodiment, three consecutive nucleotides at the 3′ terminus of (N′)y comprise the 2′OMe modification.

In some embodiments of Structure (C), in (N′)y 2, 3, 4, 5, 6, 7, 8, 9, 10, 11, 12, 13 or 14 consecutive ribonucleotides at either or 2-8 modified nucleotides at each of the 5′ and 3′ termini are independently bicyclic nucleotide. In various embodiments the bicyclic nucleotide is a locked nucleic acid (LNA). A 2′-O, 4′-C-ethylene-bridged nucleic acid (ENA) is a species of LNA (see below).

In various embodiments (N′)y comprises modified nucleotides at the 5′ terminus or at both the 3′ and 5′ termini.

In some embodiments of Structure (C), at least two nucleotides at either or both the 5′ and 3′ termini of (N′)y are joined by P-ethoxy backbone modifications. In certain preferred embodiments x=y=19 or x=y=23; in (N)x the nucleotides alternate between 2′OMe sugar modified ribonucleotides and unmodified ribonucleotides, and the ribonucleotide located at the middle position of (N)x being unmodified; and four consecutive nucleotides at the 3′ terminus or at the 5′ terminus of (N′)y are joined by three P-ethoxy backbone modifications. In another preferred embodiment, three consecutive nucleotides at the 3′ terminus or at the 5′ terminus of (N′)y are joined by two P-ethoxy backbone modifications.

In some embodiments of Structure (C), in (N′)y 2, 3, 4, 5, 6, 7 or 8, consecutive ribonucleotides at each of the 5′ and 3′ termini are independently mirror nucleotides, nucleotides joined by 2′-5′ phosphodiester bond, 2′ sugar modified nucleotides or bicyclic nucleotide. In one embodiment, the modification at the 5′ and 3′ termini of (N′)y is identical. In one preferred embodiment, four consecutive nucleotides at the 5′ terminus of (N′)y are joined by three 2′-5′ phosphodiester bonds and three consecutive nucleotides at the 3′ terminus of (N′)y are joined by two 2′-5′ phosphodiester bonds. In another embodiment, the modification at the 5′ terminus of (N′)y is different from the modification at the 3′ terminus of (N′)y. In one specific embodiment, the modified nucleotides at the 5′ terminus of (N′)y are mirror nucleotides and the modified nucleotides at the 3′ terminus of (N′)y are joined by 2′-5′ phosphodiester bond. In another specific embodiment, three consecutive nucleotides at the 5′ terminus of (N′)y are LNA nucleotides and three consecutive nucleotides at the 3′ terminus of (N′)y are joined by two 2′-5′ phosphodiester bonds. In (N)x the nucleotides alternate between 2′OMe sugar modified ribonucleotides and unmodified ribonucleotides, and the ribonucleotide located at the middle of (N)x being unmodified, or the ribonucleotides in (N)x being unmodified

In another embodiment of Structure (C), the present application provides a compound wherein x=y=19 or x=y=23; in (N)x the nucleotides alternate between 2′OMe sugar modified ribonucleotides and unmodified ribonucleotides, and the ribonucleotide located at the middle of (N)x being unmodified; three nucleotides at the 3′ terminus of (N′)y are joined by two 2′-5′ phosphodiester bonds and three nucleotides at the 5′ terminus of (N′)y are LNA such as ENA.

In another embodiment of Structure (C), five consecutive nucleotides at the 5′ terminus of (N′)y comprise the 2′OMe sugar modification and two consecutive nucleotides at the 3′ terminus of (N′)y are L-DNA.

In yet another embodiment, the present application provides a compound wherein x=y=19 or x=y=23; (N)x consists of unmodified ribonucleotides; three consecutive nucleotides at the 3′ terminus of (N′)y are joined by two 2′-5′ phosphodiester bonds and three consecutive nucleotides at the 5′ terminus of (N′)y are LNA such as ENA.

According to other embodiments of Structure (C), in (N′)y the 5′ or 3′ terminal nucleotide, or 2, 3, 4, 5 or 6 consecutive nucleotides at either termini or 1-4 modified nucleotides at each of the 5′ and 3′ termini are independently phosphonocarboxylate or phosphinocarboxylate nucleotides (PACE nucleotides). In some embodiments the PACE nucleotides are deoxyribonucleotides. In some preferred embodiments in (N′)y, 1 or 2 consecutive nucleotides at each of the 5′ and 3′ termini are PACE nucleotides.

In additional embodiments, the present application provides a compound having Structure (D):

| | | |
|---|---|---|
| (D) | 5′ (N)x-Z 3′ | antisense strand |
| | 3′ Z′-(N′)y 5′ | sense strand |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)x and (N′)y is an oligomer in which each consecutive nucleotide is joined to the next nucleotide by a covalent bond;

each of x and y is an integer between 18 and 40;

wherein (N)x comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein (N′)y comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein in each of (N)x and (N′)y modified and unmodified nucleotides are not alternating;

wherein each of Z and Z′ may be present or absent, but if present is 1-5 deoxyribonucleotides covalently attached at the 3′ terminus of any oligomer to which it is attached;

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In one embodiment of Structure (D), x=y=19 or x=y=23; (N)x comprises unmodified ribonucleotides in which two consecutive nucleotides linked by one 2′-5′ internucleotide linkage at the 3′ terminus; and (N′)y comprises unmodified ribonucleotides in which two consecutive nucleotides linked by one 2′-5′ internucleotide linkage at the 5′ terminus.

In some embodiments, x=y=19 or x=y=23; (N)x comprises unmodified ribonucleotides in which three consecutive nucleotides at the 3′ terminus are joined together by two 2′-5′ phosphodiester bonds; and (N′)y comprises unmodified ribonucleotides in which four consecutive nucleotides at the 5′ terminus are joined together by three 2′-5′ phosphodiester bonds (set forth herein as Structure II).

According to various embodiments of Structure (D) 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8 , 9, 10, 11, 12, 13 or 14 consecutive ribonucleotides starting at the ultimate or penultimate position of the 5′ terminus of (N′)y are linked by 2′-5′ internucleotide linkages.

According to one preferred embodiment of Structure (D), four consecutive nucleotides at the 5′ terminus of (N′)y are joined by three 2′-5′ phosphodiester bonds and three consecutive nucleotides at the 3′ terminus of (N′)x are joined by two 2′-5′ phosphodiester bonds. Three nucleotides at the 5′ terminus of (N′)y and two nucleotides at the 3′ terminus of (N′)x may also comprise 3′-O-methyl modifications.

According to various embodiments of Structure (D), 2, 3, 4, 5, 6, 7, 8, 9, 10, 11, 12, 13 or 14 consecutive nucleotides starting at the ultimate or penultimate position of the 3′ terminus of (N)x and 2, 3, 4, 5, 6, 7, 8 , 9, 10, 11, 12, 13 or 14 consecutive ribonucleotides starting at the ultimate or penultimate position of the 5′ terminus of (N′)y are independently mirror nucleotides. In some embodiments the mirror is an L-ribonucleotide. In other embodiments the mirror nucleotide is L-deoxyribonucleotide.

In other embodiments of Structure (D), 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8, 9, 10, 11, 12, 13 or 14 consecutive ribonucleotides starting at the ultimate or penultimate position of the 5′ terminus of (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In one preferred embodiment of Structure (D), five consecutive nucleotides at the 5′ terminus of (N′)y comprise the 2′OMe modification and five consecutive nucleotides at the 3′ terminus of (N′)x comprise the 2′OMe modification. In another preferred embodiment of Structure (D), ten consecutive nucleotides at the 5′ terminus of (N′)y comprise the 2′OMe modification and five consecutive nucleotides at the 3′ terminus of (N′)x comprise the 2′OMe modification. In another preferred embodiment of Structure (D), thirteen consecutive nucleotides at the 5′ terminus of (N′)y comprise the 2′OMe modification and five consecutive nucleotides at the 3′ terminus of (N′)x comprise the 2′OMe modification.

In some embodiments of Structure (D), in (N′)y 2, 3, 4, 5, 6, 7, 8, 9, 10, 11, 12, 13 or 14 consecutive ribonucleotides starting at the ultimate or penultimate position of the 3′ terminus of (N)x and 2, 3, 4, 5, 6, 7, 8 , 9, 10, 11, 12, 13 or 14 consecutive ribonucleotides starting at the ultimate or penultimate position of the 5′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (D), (N′)y comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In various embodiments of Structure (D), (N)x comprises a modified nucleotide selected from a bicyclic nucleotide, a 2′ sugar modified nucleotide, a mirror nucleotide, an altritol nucleotide or a nucleotide joined to an adjacent nucleotide by an internucleotide linkage selected from a 2′-5′ phosphodiester bond, a P-alkoxy linkage or a PACE linkage;

In embodiments wherein each of the 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In one specific embodiment of Structure (D), five consecutive nucleotides at the 5′ terminus of (N′)y comprise the 2′OMe modification and two consecutive nucleotides at the 3′ terminus of (N′)y are L-DNA. In addition, the compound may further comprise five consecutive 2′OMe sugar modified nucleotides at the 3′ terminus of (N′)x.

In various embodiments of Structure (D), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present application provides a compound having Structure (E):

| | | |
|---|---|---|
| (E) | 5′ (N)x-Z | 3′ antisense strand |
| | 3′ Z′-(N′)y | 5′ sense strand |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)x and (N′)y is an oligomer in which each consecutive nucleotide is joined to the next nucleotide by a covalent bond;

each of x and y is an integer between 18 and 40;

wherein (N)x comprises unmodified ribonucleotides further comprising one modified nucleotide at the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein (N′)y comprises unmodified ribonucleotides further comprising one modified nucleotide at the 3′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein in each of (N)x and (N′)y modified and unmodified nucleotides are not alternating;

wherein each of Z and Z′ may be present or absent, but if present is 1-5 deoxyribonucleotides covalently attached at the 3′ terminus of any oligomer to which it is attached;

wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In certain preferred embodiments the ultimate nucleotide at the 5′ terminus of (N)x is unmodified.

According to various embodiments of Structure (E) 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are linked by 2′-5′ internucleotide linkages.

According to various embodiments of Structure (E), 2, 3, 4, 5, 6, 7, 8, 9, 10, 11, 12, 13 or 14 consecutive nucleotides starting at the ultimate or penultimate position of the 5′ terminus of (N)x, preferably starting at the 5′ penultimate position, and 2, 3, 4, 5, 6, 7, 8 , 9, 10, 11, 12, 13 or 14 consecutive nucleotides starting at the ultimate or penultimate position of the 3′ terminus of (N′)y are independently mirror nucleotides. In some embodiments the mirror is an L-ribonucleotide. In other embodiments the mirror nucleotide is L-deoxyribonucleotide.

In other embodiments of Structure (E), 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In some embodiments of Structure (E), in (N′)y 2, 3, 4, 5, 6, 7, 8, 9, 10, 11, 12, 13 or 14 consecutive ribonucleotides starting at the ultimate or penultimate position of the 5′ terminus of (N)x, preferably starting at the 5′ penultimate position, and 2, 3, 4, 5, 6, 7, 8, 9, 10, 11, 12, 13 or 14 consecutive ribonucleotides starting at the ultimate or penultimate position of the 3′ terminus of (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (E), (N′)y comprises modified nucleotides selected from a bicyclic nucleotide, a 2′ sugar modified nucleotide, a mirror nucleotide, an altritol nucleotide, a nucleotide joined to an adjacent nucleotide by a P-alkoxy backbone modification or a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In various embodiments of Structure (E), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where both 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (E), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present application provides a compound having Structure (F):

| | | |
|---|---|---|
| (F) | 5′ (N)x-Z | 3′ antisense strand |
| | 3′ Z′-(N′)y | 5′ sense strand |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)x and (N′)y is an oligomer in which each consecutive nucleotide is joined to the next nucleotide by a covalent bond;

each of x and y is an integer between 18 and 40;

wherein each of (N)x and (N′)y comprise unmodified ribonucleotides in which each of (N)x and (N′)y independently comprise one modified nucleotide at the 3′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a nucleotide joined to an adjacent nucleotide by a 2′-5′ phosphodiester bond;

wherein in each of (N)x and (N′)y modified and unmodified nucleotides are not alternating;

wherein each of Z and Z′ may be present or absent, but if present is 1-5 deoxyribonucleotides covalently attached at the 3′ terminus of any oligomer to which it is attached;

wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In some embodiments of Structure (F), x=y=19 or x=y=23; (N′)y comprises unmodified ribonucleotides in which two consecutive nucleotides at the 3′ terminus comprises two consecutive mirror deoxyribonucleotides; and (N)x comprises unmodified ribonucleotides in which one nucleotide at the 3′ terminus comprises a mirror deoxyribonucleotide (set forth as Structure III).

According to various embodiments of Structure (F) 2, 3, 4, 5, 6, 7, 8, 9, 10, 11, 12, 13 or 14 consecutive ribonucleotides independently beginning at the ultimate or penultimate position of the 3′ termini of (N)x and (N′)y are linked by 2′-5′ internucleotide linkages.

According to one preferred embodiment of Structure (F), three consecutive nucleotides at the 3′ terminus of (N′)y are joined by two 2′-5′ phosphodiester bonds and three consecutive nucleotides at the 3′ terminus of (N′)x are joined by two 2′-5′ phosphodiester bonds.

According to various embodiments of Structure (F), 2, 3, 4, 5, 6, 7, 8, 9, 10, 11, 12, 13 or 14 consecutive nucleotides independently beginning at the ultimate or penultimate position of the 3′ termini of (N)x and (N′)y are independently mirror nucleotides. In some embodiments the mirror nucleotide is an L-ribonucleotide. In other embodiments the mirror nucleotide is an L-deoxyribonucleotide.

In other embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe).

In some embodiments of Structure (F), 2, 3, 4, 5, 6, 7, 8, 9, 10, 11, 12, 13 or 14 consecutive ribonucleotides independently beginning at the ultimate or penultimate position of the 3′ termini of (N)x and (N′)y are independently a bicyclic nucleotide. In various embodiments the bicyclic nucleotide is a locked nucleic acid (LNA) such as a 2′-O, 4′-C-ethylene-bridged nucleic acid (ENA).

In various embodiments of Structure (F), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at both the 3′ and 5′ termini.

In various embodiments of Structure (F), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 3′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (F), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present application provides a compound having Structure (G):

| | | |
|---|---|---|
| (G) | 5′ (N)x-Z | 3′ antisense strand |
| | 3′ Z′-(N′)y | 5′ sense strand |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)x and (N′)y is an oligomer in which each consecutive nucleotide is joined to the next nucleotide by a covalent bond;

each of x and y is an integer between 18 and 40;

wherein each of (N)x and (N′)y comprise unmodified ribonucleotides in which each of (N)x and (N′)y independently comprise one modified nucleotide at the 5′ terminal or penultimate position wherein the modified nucleotide is selected from the group consisting of a bicyclic nucleotide, a 2′ sugar modified nucleotide, a mirror nucleotide, a nucleotide joined to an adjacent nucleotide by a P-alkoxy backbone modification or a nucleotide joined to an adjacent nucleotide by a 2′-5′ phosphodiester bond;

wherein for (N)x the modified nucleotide is preferably at penultimate position of the 5′ terminal;

wherein in each of (N)x and (N′)y modified and unmodified nucleotides are not alternating;

wherein each of Z and Z′ may be present or absent, but if present is 1-5 deoxyribonucleotides covalently attached at the 3′ terminus of any oligomer to which it is attached;

wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In some embodiments of Structure (G), x=y=19 or x=y=23.

According to various embodiments of Structure (G) 2, 3, 4, 5, 6, 7, 8, 9, 10, 11, 12, 13 or 14 consecutive ribonucleotides independently beginning at the ultimate or penultimate position of the 5′ termini of (N)x and (N′)y are linked by 2′-5′ internucleotide linkages. For (N)x the modified nucleotides preferably starting at the penultimate position of the 5′ terminal.

According to various embodiments of Structure (G), 2, 3, 4, 5, 6, 7, 8, 9, 10, 11, 12, 13 or 14 consecutive nucleotides independently beginning at the ultimate or penultimate position of the 5′ termini of (N)x and (N′)y are independently mirror nucleotides. In some embodiments the mirror nucleotide is an L-ribonucleotide. In other embodiments the mirror nucleotide is an L-deoxyribonucleotide. For (N)x the modified nucleotides preferably starting at the penultimate position of the 5′ terminal.

In other embodiments of Structure (G), 2, 3, 4, 5, 6, 7, 8 , 9, 10, 11, 12, 13 or 14 consecutive ribonucleotides independently beginning at the ultimate or penultimate position of the 5′ termini of (N)x and (N′)y are independently 2′ sugar modified nucleotides. In some embodiments the 2′ sugar modification comprises the presence of an amino, a fluoro, an alkoxy or an alkyl moiety. In certain embodiments the 2′ sugar modification comprises a methoxy moiety (2′-OMe). In some preferred embodiments the consecutive modified nucleotides preferably begin at the penultimate position of the 5′ terminus of (N)x.

In one preferred embodiment of Structure (G), five consecutive ribonucleotides at the 5′ terminus of (N′)y comprise a 2′OMe modification and one ribonucleotide at the 5′ penultimate position of (N′)x comprises a 2′OMe modification. In another preferred embodiment of Structure (G), five consecutive ribonucleotides at the 5′ terminus of (N′)y comprise a 2′OMe modification and two consecutive ribonucleotides at the 5′ terminal position of (N′)x comprise a 2′OMe modification.

In some embodiments of Structure (G), 2, 3, 4, 5, 6, 7, 8, 9, 10, 11, 12, 13 or 14 consecutive ribonucleotides independently beginning at the ultimate or penultimate position of the 5′ termini of (N)x and (N′)y are bicyclic nucleotides. In various embodiments the bicyclic nucleotide is a locked nucleic acid (LNA) such as a 2′-O, 4′-C-ethylene-bridged nucleic acid (ENA). In some preferred embodiments the consecutive modified nucleotides preferably begin at the penultimate position of the 5′ terminus of (N)x.

In various embodiments of Structure (G), (N′)y comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In various embodiments of Structure (G), (N)x comprises a modified nucleotide selected from a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage at the 5′ terminus or at each of the 3′ and 5′ termini.

In one embodiment where each of 3′ and 5′ termini of the same strand comprise a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond. In various embodiments of Structure (G), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In additional embodiments, the present application provides a compound having Structure (H):

| | | |
|---|---|---|
| (H) | 5′ (N)x-Z | 3′ antisense strand |
| | 3′ Z′-(N′)y | 5′ sense strand |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)x and (N′)y is an oligomer in which each consecutive nucleotide is joined to the next nucleotide by a covalent bond;

each of x and y is an integer between 18 and 40;

wherein (N)x comprises unmodified ribonucleotides further comprising one modified nucleotide at the 3′ terminal or penultimate position or the 5′ terminal or penultimate position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein (N′)y comprises unmodified ribonucleotides further comprising one modified nucleotide at an internal position, wherein the modified nucleotide is selected from the group consisting of a bicyclic nucleotide, a 2′ sugar modified nucleotide, a mirror nucleotide, an altritol nucleotide, or a nucleotide joined to an adjacent nucleotide by an internucleotide linkage selected from a 2′-5′ phosphodiester bond, a P-alkoxy linkage or a PACE linkage;

wherein in each of (N)x and (N′)y modified and unmodified nucleotides are not alternating;

wherein each of Z and Z′ may be present or absent, but if present is 1-5 deoxyribonucleotides covalently attached at the 3′ terminus of any oligomer to which it is attached;

wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA set forth in any one of SEQ ID NOS:1-23.

Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In one embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or both termini of (N)x are independently 2′ sugar modified nucleotides, bicyclic nucleotides, mirror nucleotides, altritol nucleotides or nucleotides joined to an adjacent nucleotide by a 2′-5′ phosphodiester bond and 2, 3, 4, 5, 6, 7, 8, 9, 10, 11, 12, 13 or 14 consecutive internal ribonucleotides in (N′)y are independently 2′ sugar modified nucleotides, bicyclic nucleotides, mirror nucleotides, altritol nucleotides or nucleotides joined to an adjacent nucleotide by a 2′-5′ phosphodiester bond. In some embodiments the 2′ sugar modification comprises the presence of an amino, a fluoro, an alkoxy or an alkyl moiety. In certain embodiments the 2′ sugar modification comprises a methoxy moiety (2′-OMe).

In another embodiment of Structure (H), 2, 3, 4, 5, 6, 7, 8, 9, 10, 11, 12, 13 or 14 consecutive ribonucleotides independently beginning at the ultimate or penultimate position of the 3′ terminus or the 5′ terminus or 2-8 consecutive nucleotides at each of 5′ and 3′ termini of (N′)y are independently 2′ sugar modified nucleotides, bicyclic nucleotides, mirror nucleotides, altritol nucleotides or nucleotides joined to an adjacent nucleotide by a 2′-5′ phosphodiester bond, and 2, 3, 4, 5, 6, 7, 8, 9, 10, 11, 12, 13 or 14 consecutive internal ribonucleotides in (N)x are independently 2′ sugar modified nucleotides, bicyclic nucleotides, mirror nucleotides, altritol nucleotides or nucleotides joined to an adjacent nucleotide by a 2′-5′ phosphodiester bond.

In one embodiment wherein each of 3′ and 5′ termini of the same strand comprises a modified nucleotide, the modification at the 5′ and 3′ termini is identical. In another embodiment, the modification at the 5′ terminus is different from the modification at the 3′ terminus of the same strand. In one specific embodiment, the modified nucleotides at the 5′ terminus are mirror nucleotides and the modified nucleotides at the 3′ terminus of the same strand are joined by 2′-5′ phosphodiester bond.

In various embodiments of Structure (H), the modified nucleotides in (N)x are different from the modified nucleotides in (N′)y. For example, the modified nucleotides in (N)x are 2′ sugar modified nucleotides and the modified nucleotides in (N′)y are nucleotides linked by 2′-5′ internucleotide linkages. In another example, the modified nucleotides in (N)x are mirror nucleotides and the modified nucleotides in (N′)y are nucleotides linked by 2′-5′ internucleotide linkages. In another example, the modified nucleotides in (N)x are nucleotides linked by 2′-5′ internucleotide linkages and the modified nucleotides in (N′)y are mirror nucleotides.

In one preferred embodiment of Structure (H), x=y=19; three consecutive ribonucleotides at the 9-11 nucleotide positions 9-11 of (N′)y comprise 2′OMe modification and five consecutive ribonucleotides at the 3′ terminal position of (N′)x comprise 2′OMe modification.

For all the above Structures (A)-(H), in various embodiments x=y and each of x and y is 19, 20, 21, 22 or 23. In certain embodiments, x=y=19. In yet other embodiments x=y=23. In additional embodiments the compound comprises modified ribonucleotides in alternating positions wherein each N at the 5′ and 3′ termini of (N)x are modified in their sugar residues and the middle ribonucleotide is not modified, e.g. ribonucleotide in position 10 in a 19-mer strand, position 11 in a 21 mer and position 12 in a 23-mer strand.

In some embodiments where x=y=21 or x=y=23 the position of modifications in the 19 mer are adjusted for the 21 and 23 mers with the proviso that the middle nucleotide of the antisense strand is preferably not modified.

In some embodiments, neither (N)x nor (N′)y are phosphorylated at the 3′ and 5′ termini. In other embodiments either or both (N)x and (N′)y are phosphorylated at the 3′ termini. In yet another embodiment, either or both (N)x and (N′)y are phosphorylated at the 3′ termini using non-cleavable phosphate groups. In yet another embodiment, either or both (N)x and (N′)y are phosphorylated at the terminal 2′ termini position using cleavable or non-cleavable phosphate groups. These particular siRNA compounds are also blunt ended and are non-phosphorylated at the termini; however, comparative experiments have shown that siRNA compounds phosphorylated at one or both of the 3′-termini have similar activity in vivo compared to the non-phosphorylated compounds.

In certain embodiments for all the above-mentioned Structures, the compound is blunt ended, for example wherein both Z and Z′ are absent. In an alternative embodiment, the compound comprises at least one 3′ overhang, wherein at least one of Z or Z′ is present. Z and Z′ independently comprises one or more covalently linked modified or non-modified nucleotides, for example inverted dT or dA; dT, LNA, mirror nucleotide and the like. In some embodiments each of Z and Z′ are independently selected from dT and dTdT. siRNA in which Z and/or Z′ is present have similar activity and stability as siRNA in which Z and Z′ are absent.

In certain embodiments for all the above-mentioned Structures, the compound comprises one or more phosphonocarboxylate and/or phosphinocarboxylate nucleotides (PACE nucleotides). In some embodiments the PACE nucleotides are deoxyribonucleotides and the phosphinocarboxylate nucleotides are phosphinoacetate nucleotides. Examples of PACE nucleotides and analogs are disclosed in U.S. Pat. Nos. 6,693,187 and 7,067,641, both incorporated herein by reference in their entirety.

In certain embodiments for all the above-mentioned Structures, the compound comprises one or more locked nucleic acids (LNA) also defined as bridged nucleic acids or bicyclic nucleotides. Preferred locked nucleic acids are 2′-O, 4′-C-ethylene nucleosides (ENA) or 2′-O, 4′-C-methylene nucleosides. Other examples of LNA and ENA nucleotides are disclosed in WO 98/39352, WO 00/47599 and WO 99/14226, all incorporated herein by reference in their entirety.

In certain embodiments for all the above-mentioned Structures, the compound comprises one or more altritol monomers (nucleotides), also defined as 1,5 anhydro-2-deoxy-D-altrito-hexitol (see for example, Allart, et al., 1998. Nucleosides & Nucleotides 17.1523-1526; Herdewijn et al., 1999. Nucleosides & Nucleotides 18:1371-1376; Fisher et al., 2007, NAR 35(4):1064-1074; all incorporated herein by reference).

The present application explicitly excludes compounds in which each of N and/or N′ is a deoxyribonucleotide (D-A, D-C, D-G, D-T). In certain embodiments (N)x and (N′)y comprise independently 1, 2, 3, 4, 5, 6, 7, 8, 9 or more deoxyribonucleotides. In certain embodiments the present application provides a compound wherein each of N is an unmodified ribonucleotide and the 3′ terminal nucleotide or 2, 3, 4, 5, 6, 7, 8, 9, 10, 11, 12, 13 or 14 consecutive nucleotides at the 3′ terminus of (N′)y are deoxyribonucleotides. In yet other embodiments each of N is an unmodified ribonucleotide and the 5′ terminal nucleotide or 2, 3, 4, 5, 6, 7, 8, 9, 10, 11, 12, 13 or 14 consecutive nucleotides at the 5′ terminus of (N′)y are deoxyribonucleotides. In further embodiments the 5′ terminal nucleotide or 2, 3, 4, 5, 6, 7, 8, or 9 consecutive nucleotides at the 5′ terminus and 1, 2, 3, 4, 5, or 6 consecutive nucleotides at the 3′ termini of (N)x are deoxyribonucleotides and each of N′ is an unmodified ribonucleotide. In yet further embodiments (N)x comprises unmodified ribonucleotides and 1 or 2, 3 or 4 consecutive deoxyribonucleotides independently at each of the 5′ and 3′ termini and 1 or 2, 3, 4, 5 or 6 consecutive deoxyribonucleotides in internal positions; and each of N′ is an unmodified ribonucleotide. In certain embodiments the 3′ terminal nucleotide or 2, 3, 4, 5, 6, 7, 8, 9, 10, 11, 12 13 or 14 consecutive nucleotides at the 3′ terminus of (N′)y and the terminal 5′ nucleotide or 2, 3, 4, 5, 6, 7, 8, 9, 10, 11, 12 13 or 14 consecutive nucleotides at the 5′ terminus of (N)x are deoxyribonucleotides. In some embodiments the 5′ terminal nucleotide of N or 2 or 3 consecutive of N and 1,2, or 3 of N′ is a deoxyribonucleotide. Certain examples of active DNA/RNA siRNA chimeras are disclosed in US patent publication 2005/0004064, and Ui-Tei, 2008 (NAR 36(7):2136-2151) incorporated herein by reference in their entirety.

Unless otherwise indicated, in preferred embodiments of the structures discussed herein the covalent bond between each consecutive N and N′ is a phosphodiester bond.

An additional novel molecule provided by the present application is an oligonucleotide comprising consecutive nucleotides wherein a first segment of such nucleotides encode a first inhibitory RNA molecule, a second segment of such nucleotides encode a second inhibitory RNA molecule, and a third segment of such nucleotides encode a third inhibitory RNA molecule. In various embodiments each of the first, the second and the third segment comprise one strand of a double stranded RNA and the first, second and third segments are joined together by a linker. In further embodiments, the oligonucleotide comprises three double stranded segments joined together by one or more linker(s).

Thus, one molecule provided by the present application is an oligonucleotide comprising consecutive nucleotides which encode three inhibitory RNA molecules; in some embodiments said oligonucleotide possess a triple stranded structure, such that three double stranded arms are linked together by one or more linker, such as any of the linkers presented hereinabove. This molecule forms a “star”-like structure, also referred to herein as RNAstar. Such structures are disclosed in PCT patent publication WO 2007/091269, assigned to the assignee of the present application and incorporated herein by reference in its entirety.

A covalent bond refers to an internucleotide linkage linking one nucleotide monomer to an adjacent nucleotide monomer. A covalent bond includes for example, a phosphodiester bond, a phosphorothioate bond, a P-alkoxy bond, a P-carboxy bond and the like. The normal internucleoside linkage of RNA and DNA is a 3′ to 5′ phosphodiester linkage. In certain preferred embodiments a covalent bond is a phosphodiester bond. Covalent bond encompasses non-phosphorous-containing internucleoside linkages, such as those disclosed in WO 2004/041924 inter alia. Unless otherwise indicated, in preferred embodiments of the structures discussed herein the covalent bond between each consecutive N and N′ is a phosphodiester bond.

For all of the structures above, in some embodiments the oligonucleotide sequence of (N)x is fully complementary to the oligonucleotide sequence of (N′)y. In other embodiments (N)x and (N′)y are substantially complementary. In certain embodiments (N)x is fully complementary to a target mRNA sequence. In other embodiments (N)x is substantially complementary to a target mRNA sequence.

In some embodiments, neither (N)x nor (N′)y are phosphorylated at the 3′ and 5′ termini. In other embodiments either or both (N)x and (N′)y are phosphorylated at the 3′ termini (3′ Pi). In yet another embodiment, either or both (N)x and (N′)y are phosphorylated at the 3′ termini with non-cleavable phosphate groups. In yet another embodiment, either or both (N)x and (N′)y are phosphorylated at the terminal 2′ termini position using cleavable or non-cleavable phosphate groups. In further embodiments, the inhibitory nucleic acid molecules of the present application comprise one or more gaps and/or one or more nicks and/or one or more mismatches. Without wishing to be bound by theory, gaps, nicks and mismatches have the advantage of partially destabilizing the nucleic acid/siRNA, so that it is more easily processed by endogenous cellular machinery such as DICER, DROSHA or RISC into its inhibitory components.

In one aspect the present application provides a compound having Structure (I) set forth below:

| | | |
|---|---|---|
| (I) | 5′ (N)x-Z | 3′ (antisense strand) |
| | 3′ Z′-(N′)y-z″ | 5′ (sense strand) |

wherein each of N and N′ is a ribonucleotide which may be unmodified or modified, or an unconventional moiety;

wherein each of (N)x and (N′)y is an oligonucleotide in which each consecutive N or N′ is joined to the next N or N′ by a covalent bond;

wherein Z and Z′ may be present or absent, but if present is independently 1-5 consecutive nucleotides covalently attached at the 3′ terminus of the strand in which it is present;

wherein z″ may be present or absent, but if present is a capping moiety covalently attached at the 5′ terminus of (N′)y;

wherein x=18 to 27;

wherein y=18 to 27;

wherein (N)x comprises modified and unmodified ribonucleotides, each modified ribonucleotide having a 2′ OMe on its sugar, wherein N at the 3′ terminus of (N)x is a modified ribonucleotide, (N)x comprises at least five alternating modified ribonucleotides beginning at the 3′ end and at least nine modified ribonucleotides in total and each remaining N is an unmodified ribonucleotide;

wherein in (N′)y at least one unconventional moiety is present, which unconventional moiety may be an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond; and

wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In some embodiments x=y=19. In other embodiments x=y=23. In some embodiments the at least one unconventional moiety is present at positions 15, 16, 17, or 18 in (N′)y. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18.

In other embodiments the unconventional moiety is an abasic moiety. In various embodiments (N′)y comprises at least five abasic ribose moieties or abasic deoxyribose moieties.

In yet other embodiments (N′)y comprises at least five abasic ribose moieties or abasic deoxyribose moieties and at least one of N′ is an LNA.

In some embodiments of Structure (IX) (N)x comprises nine alternating modified ribonucleotides. In other embodiments of Structure (I) (N)x comprises nine alternating modified ribonucleotides further comprising a 2′O modified nucleotide at position 2. In some embodiments (N)x comprises 2′OMe sugar modified ribonucleotides at the odd numbered positions 1, 3, 5, 7, 9, 11, 13, 15, 17, 19. In other embodiments (N)x further comprises a 2′ OMe sugar modified ribonucleotide at one or both of positions 2 and 18. In yet other embodiments (N)x comprises 2′OMe sugar modified ribonucleotides at positions 2, 4, 6, 8, 11, 13, 15, 17, 19.

In various embodiments z″ is present and is selected from an abasic ribose moiety, a deoxyribose moiety; an inverted abasic ribose moiety, a deoxyribose moiety; C6-amino-Pi; a mirror nucleotide.

In another aspect the present application provides a compound having Structure (J) set forth below:

| | | |
|---|---|---|
| (J) | 5′ (N)x-Z | 3′ (antisense strand) |
| | 3′ Z′-(N′)y-z″ | 5′ (sense strand) |

wherein each of N and N′ is a ribonucleotide which may be unmodified or modified, or an unconventional moiety;

wherein each of (N)x and (N′)y is an oligonucleotide in which each consecutive N or N′ is joined to the next N or N′ by a covalent bond;

wherein Z and Z′ may be present or absent, but if present is independently 1-5 consecutive nucleotides covalently attached at the 3′ terminus of the strand in which it is present;

wherein z″ may be present or absent but if present is a capping moiety covalently attached at the 5′ terminus of (N′)y;

wherein x=18 to 27;

wherein y=18 to 27;

wherein (N)x comprises modified or unmodified ribonucleotides, and optionally at least one unconventional moiety;

wherein in (N′)y at least one unconventional moiety is present, which unconventional moiety may be an abasic ribose moiety, an abasic deoxyribose moiety, a modified or unmodified deoxyribonucleotide, a mirror nucleotide, a non-base pairing nucleotide analog or a nucleotide joined to an adjacent nucleotide by a 2′-5′ internucleotide phosphate bond; and

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In some embodiments x=y=19. In other embodiments x=y=23. In some preferred embodiments (N)x comprises modified and unmodified ribonucleotides, and at least one unconventional moiety.

In some embodiments in (N)x the N at the 3′ terminus is a modified ribonucleotide and (N)x comprises at least 8 modified ribonucleotides. In other embodiments at least 5 of the at least 8 modified ribonucleotides are alternating beginning at the 3′ end. In some embodiments (N)x comprises an abasic moiety in one of positions 5, 6, 7, 8, 9, 10, 11, 12, 13, 14 or 15.

In some embodiments the at least one unconventional moiety in (N′)y is present at positions 15, 16, 17, or 18. In some embodiments the unconventional moiety is selected from a mirror nucleotide, an abasic ribose moiety and an abasic deoxyribose moiety. In some preferred embodiments the unconventional moiety is a mirror nucleotide, preferably an L-DNA moiety. In some embodiments an L-DNA moiety is present at position 17, position 18 or positions 17 and 18. In other embodiments the at least one unconventional moiety in (N′)y is an abasic ribose moiety or an abasic deoxyribose moiety.

In various embodiments of Structure (J) z″ is present and is selected from an abasic ribose moiety, a deoxyribose moiety; an inverted abasic ribose moiety, a deoxyribose moiety; C6-amino-Pi; a mirror nucleotide.

In yet another aspect the present application provides a compound having Structure (K) set forth below:

| | | |
|---|---|---|
| (K) | 5′ (N)ₓ-Z | 3′ (antisense strand) |
| | 3′ Z′-(N′)_{y}-z″ | 5′ (sense strand) |

wherein each of N and N′ is a ribonucleotide which may be unmodified or modified, or an unconventional moiety;

wherein each of (N)x and (N′)y is an oligonucleotide in which each consecutive N or N′ is joined to the next N or N′ by a covalent bond;

wherein Z and Z′ may be present or absent, but if present is independently 1-5 consecutive nucleotides covalently attached at the 3′ terminus of the strand in which it is present;

wherein z″ may be present or absent but if present is a capping moiety covalently attached at the 5′ terminus of (N′)y;

wherein x=18 to 27;

wherein y=18 to 27;

wherein (N)x comprises a combination of modified or unmodified ribonucleotides and unconventional moieties, any modified ribonucleotide having a 2′OMe on its sugar;

wherein (N′)y comprises modified or unmodified ribonucleotides and optionally an unconventional moiety, any modified ribonucleotide having a 2′OMe on its sugar;

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In some embodiments x=y=19. In other embodiments x=y=23. In some preferred embodiments the at least one preferred one unconventional moiety is present in (N)x and is an abasic ribose moiety or an abasic deoxyribose moiety. In other embodiments the at least one unconventional moiety is present in (N)x and is a non-base pairing nucleotide analog. In various embodiments (N′)y comprises unmodified ribonucleotides. In some embodiments (N)x comprises at least five abasic ribose moieties or abasic deoxyribose moieties or a combination thereof In certain embodiments (N)x and/or (N′)y comprise modified ribonucleotides which do not base pair with corresponding modified or unmodified ribonucleotides in (N′)y and/or (N)x.

In various embodiments the present application provides an siRNA set forth in Structure (L):

| | | |
|---|---|---|
| (L) | 5′ (N)ₓ-Z | 3′ (antisense strand) |
| | 3′ Z′-(N′)_{y} | 5′ (sense strand) |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)ₓ and (N′)_{y} is an oligonucleotide in which each consecutive N or N′ is joined to the next N or N′ by a covalent bond;

wherein Z and Z′ are absent;

wherein x=y=19;

wherein in (N′)y the nucleotide in at least one of positions 15, 16, 17, 18 and 19 comprises a nucleotide selected from an abasic unconventional moiety, a mirror nucleotide, a deoxyribonucleotide and a nucleotide joined to an adjacent nucleotide by a 2′-5′ internucleotide bond;

wherein (N)x comprises alternating 2′OMe sugar modified ribonucleotides and unmodified ribonucleotides and the ribonucleotide located at the middle position of (N)x being modified or unmodified, preferably unmodified; and

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In some embodiments of Structure (L), in (N′)y the nucleotide in one or both of positions 17 and 18 comprises a modified nucleotide selected from an abasic unconventional moiety, a mirror nucleotide and a nucleotide joined to an adjacent nucleotide by a 2′-5′ internucleotide bond. In some embodiments the mirror nucleotide is selected from L-DNA and L-RNA. In various embodiments the mirror nucleotide is L-DNA.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide or pseudo nucleotide at position 2, wherein the pseudo nucleotide may be an abasic unconventional moiety and the modified nucleotide is optionally a mirror nucleotide.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)x further comprises 2′O-Me modified ribonucleotides at one or both positions 2 and 18. In other embodiments (N)x comprises 2′ OMe sugar modified ribonucleotides at positions 2, 4, 6, 8, 11, 13, 15, 17, 19.

Other embodiments of Structures (L), (I) and (J) are envisaged wherein x=y=21 or wherein x=y=23; in these embodiments the modifications for (N′)y discussed above instead of being in positions 17 and 18 are in positions 19 and 20 for 21-mer oligonucleotide and 21 and 22 for 23 mer oligonucleotide; similarly the modifications in positions 15, 16, 17, 18 or 19 are in positions 17, 18, 19, 20 or 21 for the 21-mer oligonucleotide and positions 19, 20, 21, 22, or 23 for the 23-mer oligonucleotide. The 2′OMe modifications on the antisense strand are similarly adjusted. In some embodiments (N)x comprises 2′OMe sugar modified ribonucleotides at the odd numbered positions (5′ to 3′; positions 1, 3, 5, 7, 9, 12, 14, 16, 18, 20 for the 21 mer oligonucleotide [nucleotide at position 11 unmodified] and 1, 3, 5, 7, 9, 11, 13, 15, 17, 19, 21, 23 for the 23 mer oligonucleotide [nucleotide at position 12 unmodified]. In other embodiments (N)x comprises 2′OMe sugar modified ribonucleotides at positions 2, 4, 6, 8, 10, 12, 14, 16, 18, 20 [nucleotide at position 11 unmodified for the 21 mer oligonucleotide and at positions 2, 4, 6, 8, 10, 13, 15, 17, 19, 21, 23 for the 23 mer oligonucleotide [nucleotide at position 12 unmodified].

In some embodiments (N′)y further comprises a 5′ terminal cap nucleotide. In various embodiments the terminal cap moiety is selected from an abasic unconventional moiety, an inverted abasic unconventional moiety, an L-DNA nucleotide, and a C6-imine phosphate (C6 amino linker with phosphate at terminus).

In other embodiments the present application provides a compound having Structure (M) set forth below:

| | |
|---|---|
| 5′ (N)ₓ-Z | 3′(antisense strand) |
| 3′ Z′-(N′)_{y} | 5′(sense strand) |

wherein each of N and N′ is selected from a pseudo-nucleotide and a nucleotide;

wherein each nucleotide is selected from an unmodified ribonucleotide, a modified ribonucleotide, an unmodified deoxyribonucleotide, a modified deoxyribonucleotide or an unconventional moiety;

wherein each of (N)ₓ and (N′)_{y} is an oligonucleotide in which each consecutive N or N′ is joined to the next N or N′ by a covalent bond;

wherein Z and Z′ are absent;

wherein x=18 to 27;

wherein y=18 to 27;

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In other embodiments the present application provides a double stranded compound having Structure (N) set forth below:

| | | |
|---|---|---|
| (N) | 5′ (N)ₓ-Z | 3′ (antisense strand) |
| | 3′ Z′-(N′)_{y} | 5′ (sense strand) |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)ₓ and (N′)_{y} is an oligonucleotide in which each consecutive N or N′ is joined to the next N or N′ by a covalent bond;

wherein Z and Z′ are absent;

wherein each of x and y is an integer between 18 and 40;

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23. wherein (N)x, (N′)y or (N)x and (N′)y comprise non base-pairing modified nucleotides such that (N)x and (N′)y form less than 15 base pairs in the double stranded compound.

In other embodiments the present application provides a compound having Structure (O) set forth below:

| | | |
|---|---|---|
| (O) | 5′ (N)ₓ-Z | 3′ (antisense strand) |
| | 3′ Z′-(N′)_{y} | 5′ (sense strand) |

wherein each of N is a nucleotide selected from an unmodified ribonucleotide, a modified ribonucleotide, an unmodified deoxyribonucleotide, a modified deoxyribonucleotide or an unconventional moiety;

wherein each of N′ is a nucleotide analog selected from a six membered sugar nucleotide, seven membered sugar nucleotide, morpholino moiety, peptide nucleic acid and combinations thereof;

wherein each of (N)ₓ and (N′)_{y} is an oligonucleotide in which each consecutive N or N′ is joined to the next N or N′ by a covalent bond;

wherein Z and Z′ are absent;

wherein each of x and y is an integer between 18 and 40;

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In other embodiments the present application provides a compound having Structure (P) set forth below:

| | | |
|---|---|---|
| (P) | 5′ (N)ₓ-Z | 3′ (antisense strand) |
| | 3′ Z′-(N′)_{y} | 5′ (sense strand) |

wherein each of N and N′ is a nucleotide selected from an unmodified ribonucleotide, a modified ribonucleotide, an unmodified deoxyribonucleotide, a modified deoxyribonucleotide or an unconventional moiety;

wherein each of (N)ₓ and (N′)_{y} is an oligonucleotide in which each consecutive N or N′ is joined to the next N or N′ by a covalent bond;

wherein Z and Z′ are absent;

wherein each of x and y is an integer between 18 and 40;

wherein one of N or N′ in an internal position of (N)x or (N′)y or one or more of N or N′ at a terminal position of (N)x or (N′)y comprises an abasic moiety or a 2′ sugar modified ribonucleotide;

wherein the sequence of (N′)_{y} is a sequence having complementarity to (N)x; and wherein the sequence of (N)ₓ comprises an antisense sequence having complementarity to about 18 to about 40 consecutive ribonucleotides in mRNA set forth in any one of SEQ ID NOS:1-23. Preferably (N)ₓ comprises an antisense sequence set forth in any one of SEQ ID NOs: 668-1311, 1812-2311, 2931-3549, 4050-4549, 5571-6391, 6892-7391, 7712-8031, 8532-9031, 9532-10031, 10532-11031, 11084-11135, 11177-11217, 11314-11409, 11584-11757, 11796-11833, 11862-11889, 12416-12941, 13442-13941, 14333-14723, 15224-15723, 15905-16085, 16514-16941, 17167-17391, 17881-18369, 18512-18653, 18973-19291, 19454-19615, 19906-20195, 20356-20515, 20847-21177, 21522-21865, 22366-22865, 23012-23157.

In some embodiments the sequence of (N′)y is fully complementary to the sequence of (N)x. In other embodiments the sequence of (N′)y is substantially complementary to the sequence of (N)x.

In some embodiments the sequence of (N)ₓ comprises an antisense sequence having full complementarity to about 18 to about 40 consecutive ribonucleotides in an mRNA set forth in any one of SEQ ID NOS:1-23. In other embodiments the sequence of (N)ₓ comprises an antisense sequence having complementarity to about 18 to about 40 consecutive ribonucleotides in an mRNA set forth in any one of SEQ ID NOS:1-23.

In various embodiments (N′)y comprises a modified nucleotide at position 15 wherein the modified nucleotide is selected from a mirror nucleotide and a deoxyribonucleotide.

In certain embodiments (N′)y further comprises a modified nucleotide at position 2 wherein the modified nucleotide is selected from a mirror nucleotide and an abasic unconventional moiety.

In various embodiments the antisense strand (N)x comprises 2′O-Me modified ribonucleotides at the odd numbered positions (5′ to 3′; positions 1, 3, 5, 7, 9, 11, 13, 15, 17, 19). In some embodiments (N)_{X} further comprises 2′O-Me modified ribonucleotides at one or both positions 2 and 18. In other embodiments (N)x comprises 2′OMe sugar modified ribonucleotides at positions 2, 4, 6, 8, 11, 13, 15, 17, 19.

The Structural motifs described above are useful with any oligonucleotide pair (sense and antisense strands) to a mammalian target gene, and preferably to one of the human genes set forth in Table 1.

Any siRNA sequence disclosed herein can be prepared having any of the modifications/structures disclosed herein.

In another aspect the present application provides a pharmaceutical composition comprising a modified or unmodified compound of the present application, in an amount effective to down-regulate a human target gene expression or over-expression wherein the compound comprises an antisense sequence, (N)_{x,}; and a pharmaceutically acceptable carrier.

In yet another aspect the present application provides a pharmaceutical composition comprising one or more of modified compounds of the present application, in an amount effective to down-regulate expression or over-expression of one or more human target genes wherein the compound comprises an antisense sequence, (N)_{x,}; and a pharmaceutically acceptable carrier.

In another aspect, the present application relates to a method for the treatment of a subject in need of prevention of or treatment for, a disease, injury or disorder or symptoms associated with the disease, injury or disorder, associated with the expression of a target gene comprising administering to the subject an amount of at least one siRNA, according to the present application, in a therapeutically effective dose so as to thereby treat the subject or so as to prevent the disease, injury or disorder from occurring.

The methods of the application comprise administering to the subject one or more siRNA compounds which down-regulate expression of a target gene. The novel structures disclosed herein, when integrated into antisense and corresponding sense nucleic acid sequences, provide siRNA compounds useful in reducing expression of the target genes.

In various embodiment the target gene is selected from NOX4, NOX1, NOX2 (gp91phox, CYBB), NOX5, DUOX2, NOXO1, NOXO2 (NCF1), NOXA1, NOXA2 (p67phox, NCF2), TP53; HTRA2; KEAP1; SHC1, ZNHIT1, LGALS3, and HI95.

In some embodiments, the present application relates to a method for the treatment of a subject in need of treatment for a disease, injury or disorder or symptom or condition associated with the disease, injury or disorder, associated with the expression of at least two target genes comprising administering to the subject at least two siRNA compounds which down-regulate or inhibit expression or over-expression of the target genes. In preferred embodiments the siRNA compounds are chemically modified according to the embodiments of the present application. In some embodiments, the siRNA compounds are administered by the same route, either from the same or from different pharmaceutical compositions. However, in other embodiments, using the same route of administration for the two or more of the therapeutic siRNA compounds either is impossible or is not preferred. Persons skilled in the art are aware of the best modes of administration for each therapeutic agent, either alone or in a combination.

Indications and Methods of Treatment

In one aspect, the present application relates to a method for the treatment of a subject in need of prevention of or treatment for, a disease, injury or disorder associated with expression or over-expression of one or more of the target genes, comprising administering to the subject an amount of at least one chemically modified siRNA which inhibits expression of one or more of the target genes disclosed herein. In certain preferred embodiments more than one siRNA compound is administered.

In preferred embodiments the subject being treated is a warm-blooded animal and, in particular, mammal including human.

The methods of the application comprise administering to the subject one or more of the siRNA compounds which down-regulate expression of one or more of the target genes; and in particular at least one siRNA in a therapeutically effective dose so as to thereby treat the subject.

Thus, in one embodiment the present application provides for a method of treating a subject suffering from or at risk of a neurodegenerative disease or disorder, including Alzheimer's Disease (AD) and Amyotrophic lateral sclerosis (ALS), a microvascular disorder, a respiratory disorder, a hearing disorder, hearing loss; acute renal failure (ARF); an ophthalmic disease including glaucoma and ION; a respiratory disease including acute respiratory distress syndrome (ARDS), chronic obstructive pulmonary disease (COPD) and other acute lung and respiratory injuries; injury (e.g. ischemia-reperfusion injury), graft dysfunction and acute rejection after organ transplantation, including lung, kidney, bone marrow, heart, pancreas, cornea or liver transplantation; nephrotoxicity; pressure sores, dry eye syndrome, oral mucositis, comprising administering to the patient a pharmaceutical composition comprising a at least one siRNA compound as disclosed herein in a therapeutically effective amount so as to thereby treat the patient. Oligonucleotide sequences of certain preferred siRNA inhibitors are set forth in any one of Tables A1-A18, Tables B1-15 and Tables C1-C2.

The term “treatment” as used herein refers to administration of a therapeutic substance to a subject in need thereof in an amount effective to ameliorate symptoms associated with a disease, an injury or a disorder, to lessen the severity or cure the disease, the injury or the disorder, to delay the onset or the progression of the disease, the injury or the disorder or to prevent the disease, the injury or the disorder from occurring.

Additionally, the application provides a method of down-regulating the expression of a mammalian gene selected from the group consisting of NOX4, NOX1, NOX2 (gp91phox, CYBB), NOX5, DUOX2, NOXO1, NOXO2, NOXA1, NOXA2 (p67phox) TP53; HTRA2; KEAP1; SHC1-SHC, ZNHIT1, LGALS3, and HI95, by at least 40%, preferably by 50%, 60% or 70%, more preferably by 75%, 80% or 90% as compared to a control, comprising contacting a target mRNA transcript selected from the group consisting of NOX4, NOX1, NOX2 (gp9lphox, CYBB), NOX5, DUOX2, NOXO1, NOXO2, NOXA1, NOXA2 (p67phox), TP53; HTRA2; KEAP1; SHC1-SHC, ZNHIT1, LGALS3, and HI95, respectively with one or more of the compounds of the present application.

In one embodiment the compound of the present application is down-regulating one or more mammalian target genes selected from the group consisting of NOX4, NOX1, NOX2 (gp91phox, CYBB), NOX5, DUOX2, NOXO1, NOXO2, NOXA1, NOXA2 (p67phox), TP53; HTRA2; KEAP1; SHC1-SHC, ZNHIT1, LGALS3, and HI95, whereby the down-regulation is selected from the group comprising down-regulation of gene function, down-regulation of polypeptide and down-regulation of mRNA expression.

In one embodiment the compound is down-regulating a mammalian target polypeptide, whereby the down-regulation is selected from the group comprising down-regulation of function (which may be examined by an enzymatic assay or a binding assay with a known interactor of the native gene/polypeptide, inter alia), down-regulation of protein (which may be examined by Western blotting, ELISA or immuno-precipitation, inter alia) and down-regulation of mRNA expression (which may be examined by Northern blotting, quantitative RT-PCR, in-situ hybridization or microarray hybridization, inter alia).

In some embodiments the application provides a method of reducing the level of target gene expression in a cell comprising contacting the cell with a compound or composition of the application in a therapeutically effective dose thereby reducing the level of the target gene. In some embodiments the application provides a method of reducing the level of at least one target gene expression in a cell comprising contacting the cell with at least one compound or at least one composition of the application in a therapeutically effective dose thereby reducing the level of at least one target gene.

In additional embodiments the application provides a method of treating a patient at risk of or suffering from, a disease accompanied by an elevated level of a mammalian target gene comprising mRNA set forth in any one of SEQ ID NOS:1-23, the method comprising administering to the patient at least one compound or composition of the application in a therapeutically effective dose thereby treating the patient.

The present application relates to the use of compounds which down-regulate the expression of a mammalian target gene particularly to novel small interfering RNAs (siRNAs), in the treatment of the following diseases or conditions in which inhibition of the expression of the mammalian gene is beneficial: neurodegenerative diseases and disorders, including Alzheimer's disease, ALS, Parkinson's Disease, multiple sclerosis and the like; spinal cord injury, brain injury, hearing loss, acute renal failure, ocular disease e.g. glaucoma, dry eye syndrome and ION, a respiratory disease including Acute Respiratory Distress Syndrome and other acute lung injuries, injury (e.g. ischemia-reperfusion injury), graft dysfunction and acute rejection after organ transplantation, including lung, kidney, bone marrow, heart, pancreas, cornea or liver transplantation; pressure sores, osteoarthritis, an ophthalmic disease including glaucoma and ION; and Chronic Obstructive Pulmonary Disease (COPD). Other indications include chemical-induced nephrotoxicity and chemical-induced neurotoxicity, for example toxicity induced by cisplatin and cisplatin—like compounds, by aminoglycosides, by loop diuretics, and by hydroquinone and their analogs.

Methods, molecules and compositions which inhibit the mammalian target gene or polypeptide are discussed herein at length, and any of said molecules and/or compositions may be beneficially employed in the treatment of a patient at risk of or suffering from any of said conditions. In certain preferred embodiments the target gene is NOX4. A list of siRNAs directed to target genes is provided in Tables A1-A18 and B1-B15, infra. The number in parenthesis (#) indicates the number of the compound in the respective table.

Certain preferred siRNA compounds are provided in Table B15, infra.

The compounds disclosed herein are preferably chemically modified according to the embodiments of the present application.

“Treatment” refers to both therapeutic treatment and prophylactic or preventative measures, wherein the object is to prevent or slow down the onset or progression of a disease related to one or more of the target genes listed in Table 1. Those in need of treatment include those already experiencing the disease or condition, those prone to having the disease or condition, and those in which the disease or condition is to be prevented. The compounds of the application may be administered before, during or subsequent to the onset of the disease or condition.

The method of the application includes administering a therapeutically effective amount of one or more compounds which down-regulate expression of one or more target genes, particularly the novel siRNAs of the present application.

In some preferred embodiments, the siRNA compounds of the application are administered in various conditions of hearing loss. Without being bound by theory, the hearing loss may be due to inner ear hair cell damage or loss, wherein the damage or loss is caused by, inter alia, infection, mechanical injury, loud sound, aging (presbycusis or loss of hearing that gradually occurs in most individuals as they grow older), or chemical-induced ototoxicity. Ototoxins include therapeutic drugs including antineoplastic agents, salicylates, quinines, and aminoglycoside antibiotics, contaminants in foods or medicinals, and environmental or industrial pollutants. Typically, treatment is performed to prevent or reduce ototoxicity, especially resulting from or expected to result from administration of therapeutic drugs. Preferably a therapeutically effective composition is given immediately after the exposure to prevent or reduce the ototoxic effect. More preferably, treatment is provided prophylactically, either by administration of the composition prior to or concomitantly with the ototoxic pharmaceutical or the exposure to the ototoxin.

By “ototoxin” in the context of the present application is meant a substance that through its chemical action injures, impairs or inhibits the activity of the sound receptors component of the nervous system related to hearing, which in turn impairs hearing (and/or balance). In the context of the present application, ototoxicity includes a deleterious effect on the inner ear hair cells Ototoxic agents that cause hearing impairments include, but are not limited to, neoplastic agents such as vincristine, vinblastine, cisplatin and cisplatin-like compounds, taxol and taxol-like compounds, dideoxy-compounds, e.g., dideoxyinosine; alcohol; metals; industrial toxins involved in occupational or environmental exposure; contaminants of food or medicinals; and over-doses of vitamins or therapeutic drugs, e.g., antibiotics such as penicillin or chloramphenicol, and megadoses of vitamins A, D, or B6, salicylates, quinines and loop diuretics.

The ototoxic effects of various therapeutic drugs on auditory cells and spiral ganglion neurons are often the limiting factor for their therapeutic usefulness. Main ototoxic drugs include the widely used chemotherapeutic agent cisplatin and its analogs, commonly used aminoglycoside antibiotics, e.g. gentamycin, for the treatment of infections caused by gram-negative bacteria, quinine and its analogs, salicylate and its analogs, and loop-diuretics.

For example, antibacterial aminoglycosides such as gentamycin, streptomycin, kanamycin, tobramycin, and the like are known to have serious toxicity, particularly ototoxicity and nephrotoxicity, which reduce the value of such antimicrobials as therapeutic agents (see Goodman and Gilman's The Pharmacological Basis of Therapeutics, 6th ed., A. Goodman Gilman et al., eds; Macmillan Publishing Co., Inc., New York, 1980, pp. 1169-71).

Clearly, ototoxicity is a dose limiting side effect of antibiotic administration. Studies have shown that from 4% to 15% of patients receiving 1 gram per day for greater than 1 week develop measurable hearing loss, which slowly becomes worse and can lead to complete permanent deafness if treatment continues.

Nephrotoxicity and ototoxicity are serious dose limiting side effect for cisplatin, a platinum coordination complex, that has proven effective on a variety of human cancers including testicular, ovarian, bladder, and head and neck cancer. Cisplatin (Platinol®) damages auditory and vestibular systems. Salicylates, such as aspirin, are the most commonly used therapeutic drugs for their anti-inflammatory, analgesic, anti-pyretic and anti-thrombotic effects. Unfortunately, they too have ototoxic side effects and can lead to tinnitus (“ringing in the ears”) and temporary hearing loss. Moreover, if the drug is used at high doses for a prolonged time, chronic and irreversible hearing impairment can become an issue. Another target organ for cisplatin toxicity is the kidney. This toxicity is manifested by reduced renal function and leads to serum electrolyte changes and pathological changes in the urine analysis. Doses of cisplatin, which produce changes in renal function may cause no histopathological changes. Higher doses of the drug lead to terstitial nephritis. Cisplatin also causes bone marrow hypoplasia, and can cause autonomic neuropathy. Slight changes in liver function and histopathology are also observed following cisplatin therapy.

Without being bound by theory, it is believed that cisplatin drugs and other potentially ototoxic drugs (such as aminoglycoside antibiotics) may induce the ototoxic effects via programmed cell death or apoptosis in inner ear tissue, particularly inner ear hair cells (Zhang et al., Neuroscience 2003, 120(1):191-205; Wang et al., J. Neuroscience 2003, 23(24):8596-8607). In mammals, auditory hair cells are produced only during embryonic development and do not regenerate if lost during postnatal life, therefore, a loss of hair cells will result in profound and irreversible deafness. Unfortunately, at present, there are no effective therapies to treat the cochlea and reverse this condition. Thus, an effective therapy to prevent cell death of auditory hair cells would be of great therapeutic value.

Another type of hearing loss is presbycusis, which is age related hearing loss. It is estimated that about 30-35 percent of adults between the ages of 65 and 75 years and about 40-50 percent of people aged 75 and older have hearing loss. Accordingly, there exists a need for means to prevent, reduce or treat the incidence and/or severity of inner ear disorders and hearing impairments involving inner ear tissue, particularly inner ear hair cells.

By “exposure to an toxic agent” is meant that the toxic agent is made available to, or comes into contact with, a mammal. A toxic agent can be toxic to one or more organs in the body, for example, the ear, kidney, nervous system, liver and the like. Exposure to an toxic agent can occur by direct administration, e.g., by ingestion or administration of a food, medicinal, or therapeutic agent, e.g., a chemotherapeutic agent, by accidental contamination, or by environmental exposure, e g., aerial or aqueous exposure.

Hearing loss relevant to the application may be due to end-organ lesions involving inner ear hair cells, e.g., acoustic trauma, viral endolymphatic labyrinthitis, Meniere's disease. Hearing impairments include tinnitus, which is a perception of sound in the absence of an acoustic stimulus, and may be intermittent or continuous, wherein there is diagnosed a sensorineural loss. Hearing loss may be due to bacterial or viral infection, such as in herpes zoster oticus, purulent labyrinthitis arising from acute otitis media, purulent meningitis, chronic otitis media, sudden deafness including that of viral origin, e.g., viral endolymphatic labyrinthitis caused by viruses including mumps, measles, influenza, chicken pox, mononucleosis and adenoviruses. The hearing loss can be congenital, such as that caused by rubella, anoxia during birth, bleeding into the inner ear due to trauma during delivery, ototoxic drugs administered to the mother, erythroblastosis fetalis, and hereditary conditions including Waardenburg's syndrome and Hurler's syndrome.

The hearing loss can be noise-induced, generally due to a noise greater than about 85 decibels (db) that damages the inner ear. In a particular aspect of the application, the hearing loss is caused by an ototoxic drug that effects the auditory portion of the inner ear, particularly inner ear hair cells. Incorporated herein by reference are chapters 196, 197, 198 and 199 of The Merck Manual of Diagnosis and Therapy, 14th Edition, (1982), Merck Sharp & Dome Research Laboratories, N.J. and corresponding chapters in the most recent 16th edition, including Chapters 207 and 210 relating to description and diagnosis of hearing and balance impairments.

It is the object of the present application to provide a method and compositions for treating a mammal, to prevent, reduce, or treat a hearing impairment, disorder or imbalance, preferably an ototoxin-induced hearing condition, by administering to a mammal in need of such treatment a composition of the application. One embodiment of the application is a method for treating a hearing disorder or impairment wherein the ototoxicity results from administration of a therapeutically effective amount of an ototoxic pharmaceutical drug. Typical ototoxic drugs are chemotherapeutic agents, e.g. antineoplastic agents, and antibiotics. Other possible candidates include loop-diuretics, quinines or a quinine-like compound, and salicylate or salicylate-like compounds.

Ototoxic aminoglycoside antibiotics include but are not limited to neomycin, paromomycin, ribostamycin, lividomycin, kanamycin, amikacin, tobramycin, viomycin, gentamycin, sisomicin, netilmicin, streptomycin, dibekacin, fortimicin, and dihydrostreptomycin, or combinations thereof. Particular antibiotics include neomycin B, kanamycin A, kanamycin B, gentamycin C1, gentamycin C1a, and gentamycin C2.

The methods and compositions of the present application are also effective in the treatment of acoustic trauma or mechanical trauma, preferably acoustic or mechanical trauma that leads to inner ear hair cell loss. Acoustic trauma to be treated in the present application may be caused by a single exposure to an extremely loud sound, or following long-term exposure to everyday loud sounds above 85 decibels. Mechanical inner ear trauma to be treated in the present application is for example the inner ear trauma following insertion and operation of an electronic device in the inner ear. The compositions of the present application prevent or minimize the damage to inner ear hair cells associated with the device.

In some embodiments the composition of the application is co-administered with an ototoxin. For example, the present application provides an improved method for treatment of infection of a mammal receiving an antibiotic for treatment of the infection, comprising administering a therapeutically effective amount of one or more compounds (particularly novel siRNAs) which down-regulate expression of the mammalian NOX4 gene, to the patient in need of such treatment to reduce or prevent ototoxin-induced hearing impairment associated with the antibiotic. The compounds which down-regulate expression of a target gene, in particular novel siRNA compounds of the application are preferably administered locally within the inner ear. In one specific embodiment the siRNA compounds target mammalian NOX4.

In yet another embodiment an improved method for treatment of cancer in a mammal by administration of a chemotherapeutic compound is provided, wherein the improvement comprises administering a therapeutically effective amount of a composition of the application to the patient in need of such treatment to reduce or prevent ototoxin-induced hearing impairment associated with the chemotherapeutic drug. The compounds which reduce or prevent the ototoxin-induced hearing impairment, e.g. the novel siRNAs inter alia are preferably administered locally within the inner ear.

In another embodiment the methods of treatment are applied to treatment of hearing loss resulting from the administration of a chemotherapeutic agent in order to treat its ototoxic side effect.

In another embodiment the methods of the application are applied to hearing impairments resulting from the administration of quinine and its synthetic substitutes, typically used in the treatment of malaria, to treat its ototoxic side effect.

In another embodiment the methods of the application are applied to hearing impairments resulting from administration of a diuretic to treat its ototoxic side effect. Diuretics, particularly “loop” diuretics, i.e. those that act primarily in the Loop of Henle, are candidate ototoxins. Illustrative examples, not limiting to the application method, include furosemide, ethacrylic acid, and mercurials. Diuretics are typically used to prevent or eliminate edema. Diuretics are also used in nonedematous states for example hypertension, hypercalcemia, idiopathic hypercalciuria, and nephrogenic diabetes insipidus.

In another preferred embodiment, the compounds of the application are used for treating acute renal failure, in particular acute renal failure due to ischemia in post surgical patients, and acute renal failure due to chemotherapy treatment such as cisplatin administration or sepsis-associated acute renal failure. A preferred use of the compounds of the application is for the prevention of acute renal failure in high-risk patients undergoing major cardiac surgery or vascular surgery. The patients at high-risk of developing acute renal failure can be identified using various scoring methods such as the Cleveland Clinic algorithm or that developed by US Academic Hospitals (QMMI) and by Veterans' Administration (CICSS). Other preferred uses of the compounds of the application are for the prevention of ischemic acute renal failure in kidney transplant patients or for the prevention of toxic ARF in patients receiving chemotherapy.

In another preferred embodiment, the compounds of the application are used for treating ocular diseases (e.g. glaucoma, ocular ischemic conditions and dry eye syndrome).

In other embodiments the compounds and methods of the application are useful for treating or preventing the incidence or severity of various diseases and conditions in a patient, in particular conditions which are result from ischemic/reperfusion injury or oxidative stress, Acute Respiratory Distress Syndrome (ARDS) for example due to coronavirus infection or endotoxins, severe acute respiratory syndrome (SARS), and other acute lung injuries, ischemia reperfusion injury associated with lung transplantation, glaucoma, spinal cord injury, pressure sores, osteoarthritis and Chronic Obstructive Pulmonary Disease (COPD). The methods comprising administering to the patient a composition comprising one or more inhibitors (such as siRNA compounds) which inhibit at least one target gene in a therapeutically effective dose, thereby treating the patient.

In other embodiments the compounds and methods of the application are useful for treating or preventing the incidence or severity of other diseases and conditions in a patient. These diseases and conditions include, without being limited to, stroke and stroke-like situations (e.g. cerebral, renal, cardiac failure), neuronal cell death, brain injuries with or without reperfusion, chronic degenerative diseases e.g. neurodegenerative disease including Alzheimer's disease, Huntington's disease, Parkinson's disease, multiple sclerosis, amyotrophic lateral sclerosis, spinobulbar atrophy, prion disease, and apoptosis resulting from traumatic brain injury (TBI).

The compounds and methods of the application are directed to providing neuroprotection, cerebroprotection, or to prevent and/or treat cytotoxic T cell and natural killer cell-mediated apoptosis associated with autoimmune disease and transplant rejection, or to prevent cell death of cardiac cells including heart failure, cardiomyopathy, viral infection or bacterial infection of the heart, myocardial ischemia, myocardial infarct, and myocardial ischemia, coronary artery by-pass graft, or to prevent and/or treat mitochondrial drug toxicity e. g. as a result of chemotherapy or HIV therapy, to prevent cell death during viral infection or bacterial infection, or to prevent and/or treat inflammation or inflammatory diseases, inflammatory bowel disease, sepsis and septic shock. Other uses include prevention of cell death from follicle to ovocyte stages, from ovocyte to mature egg stages and sperm (for example, methods of freezing and transplanting ovarian tissue, artificial fertilization), or to preserve fertility in mammals after chemotherapy, in particular human mammals, or to prevent and/or treat, macular degeneration, or to prevent and/or treat acute hepatitis, chronic active hepatitis, hepatitis-B, and hepatitis-C, or to prevent hair loss, (e.g. hair loss due-to male-pattern baldness, or hair loss due to radiation, chemotherapy or emotional stress), or to treat or ameliorate skin damage whereby the skin damage may be due to exposure to high levels of radiation, heat, chemicals, sun, or to burns and autoimmune diseases), or to prevent cell death of bone marrow cells in myelodysplastic syndromes (MDS), or to treat pancreatisis, or to treat, rheumatoid arthritis, psoriasis, glomerulonephritis, atherosclerosis, and graft versus host disease (GVHD), or to treat retinal pericyte apoptosis, retinal damages resulting from ischemia, diabetic retinopathy, or to treat any disease states associated expression of at least one target gene selected from NOX4, NOX1, NOX2 (gp91phox, CYBB), NOX5, DUOX2, NOXO1, NOXO2 (NCF1), NOXA1, NOXA2 (p67phox, NCF2), TP53; HTRA2; KEAP1; SHC1, ZNHIT1, LGALS3, and HI95.

The present application also relates to organ transplantation in general. For organ transplantation, either the donor or the recipient or both are treated with a compound or composition of the present application. Accordingly, the present application relates to a method of treating an organ donor and/or an organ recipient comprising the step of administering to the organ donor and/or organ recipient a therapeutically effective amount of a compound according to the present application. In some embodiments the compounds of the present application are useful in preventing delayed graft function following a cadaveric organ transplant such as kidney transplant.

The application further relates to a method for preserving an organ comprising contacting the organ with an effective amount of compound of the present application. Also provided is a method for reducing or preventing injury (in particular reperfusion injury) of an organ during surgery and/or following removal of the organ from a subject comprising placing the organ in an organ preserving solution wherein the solution comprises a compound according to the present application.

The methods and compositions of the present application are effective in the treatment and prevention of any chronic wounds including inter alia pressure sores, venous ulcers, and diabetic ulcers. In all these chronic wound types, the underlying precipitating event is a period of ischemia followed by a period of reperfusion. These ischemia-reperfusion events are usually repetitive, which means the deleterious effects of ischemia-reperfusion are potentiated and eventually sufficient to cause ulceration. For both pressure sores and diabetic foot ulcers, the ischemic event is the result of prolonged pressure sufficient to prevent tissue perfusion, and when the pressure is finally relieved, the reperfusion injury occurs. The present compositions are effective in inhibiting the damage caused by ischemia-reperfusion in chronic wounds.

The present compositions are also effective in other conditions associated with ischemia-reperfusion such as but not limited to: organ transplantation, intestinal and colon anastamoses, operations on large blood vessels, stitching detached limbs, balloon angioplasty or any cardiac surgery, stroke or brain trauma, limb transplantation, pulmonary hypertension, hypoxemia, and noncardiogenic pulmonary edema, acute renal failure, acute glaucoma, diabetic retinopathy, hypertensive retinopathy, and retinal vascular occlusion, cochlear ischemia, microvascular surgery and ischemic lesions in scleroderma.

“Treating a subject suffering from a disease, an injury or a disorder” refers to administering a therapeutic siRNA substance effective to ameliorate symptoms associated with a disease, an injury or a disorder, to lessen the severity or cure the disease, the injury or the disorder, to delay onset or progression of the disease, the injury or the disorder, or to prevent the disease, the injury or the disorder from occurring.” Treatment” refers to both therapeutic treatment and prophylactic or preventative measures, wherein the object is to prevent or slow down (lessen) a disease or disorder.

A “therapeutically effective dose” refers to an amount of a pharmaceutical compound or composition which is effective to achieve an improvement in a patient or his physiological systems including, but not limited to, improved survival rate, more rapid recovery, or improvement or elimination of symptoms, and other indicators as are selected as appropriate determining measures by those skilled in the art.

“Respiratory disorder” refers to conditions, diseases or syndromes of the respiratory system including but not limited to pulmonary disorders of all types including chronic obstructive pulmonary disease (COPD), acute lung injury (ALI), emphysema, chronic bronchitis, asthma and lung cancer, inter alia. Emphysema and chronic bronchitis may occur as part of COPD or independently. Conditions resulting from lung transplantation may also be viewed as such.

“Ischemic diseases/conditions” relates to any disease in which ischemia is involved, as well as ischemia-reperfusion injury and ischemia in connection with organ transplantation.

“Microvascular disorder” refers to any condition that affects microscopic capillaries and lymphatics, in particular vasospastic diseases, vasculitic diseases and lymphatic occlusive diseases. Examples of microvascular disorders include, inter alia: eye disorders such as Amaurosis Fugax (embolic or secondary to SLE), apla syndrome, Prot CS and ATIII deficiency, microvascular pathologies caused by IV drug use, dysproteinemia, temporal arteritis, anterior ischemic optic neuropathy, optic neuritis (primary or secondary to autoimmune diseases), glaucoma, von Hippel Lindau syndrome, corneal disease, corneal transplant rejection cataracts, Eales' disease, frosted branch angiitis, encircling buckling operation, uveitis including pars planitis, choroidal melanoma, choroidal hemangioma, optic nerve aplasia; retinal conditions such as retinal artery occlusion, retinal vein occlusion, retinopathy of prematurity, HIV retinopathy, Purtscher retinopathy, retinopathy of systemic vasculitis and autoimmune diseases, diabetic retinopathy, hypertensive retinopathy, radiation retinopathy, branch retinal artery or vein occlusion, idiopathic retinal vasculitis, aneurysms, neuroretinitis, retinal embolization, acute retinal necrosis, Birdshot retinochoroidopathy, long-standing retinal detachment; systemic conditions such as Diabetes mellitus, diabetic retinopathy (DR), diabetes-related microvascular pathologies (as detailed herein), hyperviscosity syndromes, aortic arch syndromes and ocular ischemic syndromes, carotid-cavernous fistula, multiple sclerosis, systemic lupus erythematosus, arteriolitis with SS-A autoantibody, acute multifocal hemorrhagic vasculitis, vasculitis resulting from infection, vasculitis resulting from Behcet's disease, sarcoidosis, coagulopathies, neuropathies, nephropathies, microvascular diseases of the kidney, acute renal failure and ischemic microvascular conditions, inter alia.

Microvascular disorders may comprise a neovascular element. The term “neovascular disorder” refers to those conditions where the formation of blood vessels (neovascularization) is harmful to the patient. Examples of ocular neovascularization include: retinal diseases (diabetic retinopathy, diabetic Macular Edema, chronic glaucoma, retinal detachment, and sickle cell retinopathy); rubeosis iritis; proliferative vitreo-retinopathy; inflammatory diseases; chronic uveitis; neoplasms (retinoblastoma, pseudoglioma and melanoma); Fuchs' heterochromic iridocyclitis; neovascular glaucoma; corneal neovascularization (inflammatory, transplantation and developmental hypoplasia of the iris); neovascularization following a combined vitrectomy and lensectomy; vascular diseases (retinal ischemia, choroidal vascular insufficiency, choroidal thrombosis and carotid artery ischemia); neovascularization of the optic nerve; and neovascularization due to penetration of the eye or contusive ocular injury. All these neovascular conditions may be treated using the compounds and pharmaceutical compositions of the present application.

Eye disease or ocular disease or ocular disorder or ophthalmic disease or ophthalmic disorder refers to refers to conditions, diseases or syndromes of the eye including but not limited to any conditions involving choroidal neovascularization (CNV), wet and dry AMD, ocular histoplasmosis syndrome, angiod streaks, ruptures in Bruch's membrane, myopic degeneration, ocular tumors, retinal degenerative diseases, glaucoma, ION, AION, NAION and retinal vein occlusion (RVO). Some conditions disclosed herein, such as DR, which may be treated according to the methods of the present application have been regarded as either a microvascular disorder and an eye disease, or both, under the definitions presented herein.

In some embodiments the compounds and methods of the application are useful for treating or preventing the incidence or severity of other diseases, injuries and conditions in a patient. These diseases, injuries and conditions include, without being limited to, stroke and stroke-like situations (e.g. cerebral, renal, cardiac failure), neuronal cell death, brain injuries with or without reperfusion, chronic degenerative diseases e.g. neurodegenerative disease including Alzheimer's disease, Huntington's disease, Parkinson's disease, multiple sclerosis, amyotrophic lateral sclerosis, spinobulbar atrophy, prion disease, and apoptosis resulting from traumatic brain injury (TBI).

The compounds and methods of the application are directed to providing neuroprotection for example in the optic nerve and cerebroprotection.

Emphysema and COPD

Among the mechanisms that underlie lung destruction in emphysema, excessive formation of reactive oxygen species (ROS) should be first of all mentioned. It is well established that prooxidant/antioxidant imbalance exists in the blood and in the lung tissue of smokers (Hulea S A, et al: 1995. J Environ Pathol Toxicol Oncol. 14(3-4):173-80; Rahman I, MacNee W. 1999. Am J Physiol. 277(6 Pt 1):L1067-88; MacNee W. 2000 Chest. 117(5 Suppl 1):303S-17S; Marwick J A, et al., 2002. Ann NY Acad Sci. 973:278-83; Aoshiba K, et al., 2003. Inhal Toxicol. (10):1029-38; Dekhuijzen P N. 2004. Eur Respir J. 23(4):629-36; Tuder R M, et al., 2003. Am J Respir Cell Mol Biol, 29:88-97). After one hour exposure of mice to CS, there is a dramatic increase of 8-hydroxy-2′-deoxyguanosine (8-OHdG) in the alveolar epithelial cells, particularly of type II (see Inhal Toxicol. 2003 15(10):1029-38, above).

Overproduced reactive oxygen species are known for their cytotoxic activity, which stems from a direct DNA damaging effect and from the activation of apoptotic signal transduction pathways (Takahashi et al., 2004. Brain Res Bull. 62(6):497-504; Taniyama Y, Griendling K K. 2003. Hypertension. 42(6):1075-81; Higuchi Y. 2003. Biochem Pharmacol. 66(8):1527-35; Punj V, Chakrabarty A M. 2003. Cell Microbiol. (4):225-31; Ueda et al., 2002 Antioxid Redox Signal. 4(3):405-14).

Acute Renal Failure

Acute renal failure (ARF) is a clinical syndrome characterized by rapid deterioration of renal function that occurs within days. The principal feature of ARF is an abrupt decline in glomerular filtration rate (GFR), resulting in the retention of nitrogenous wastes (urea, creatinine) Worldwide, severe ARF occurs in about 170-200 per million population annually. To date, there is no specific treatment for established ARF. Several drugs have been found to ameliorate toxic and ischemic experimental ARF, as manifested by lower serum creatinine levels, reduced histological damage and faster recovery of renal function in different animal models. These include anti-oxidants, calcium channel blockers, diuretics, vasoactive substances, growth factors, anti-inflammatory agents and more. However, the drugs tested in clinical trials showed no benefit, and their use in clinical ARF has not been approved.

In the majority of hospitalized ARF patients, ARF is caused by acute tubular necrosis (ATN), which results from ischemic and/or nephrotoxic insults. Renal hypoperfusion is caused by hypovolemic, cardiogenic and septic shock, by administration of vasoconstrictive drugs or renovascular injury. Nephrotoxins include exogenous toxins such as contrast media, aminoglycosides and cisplatin and cisplatin-like compounds as well as endogenous toxin such as myoglobin. Recent studies, however, support the theory that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum. It has been suggested that apoptotic tubule cell death may be more predictive of functional changes than necrotic cell death (Komarov et al., Science 1999, 10;285(5434):1733-7); Supavekin et al., Kidney Int. 2003, 63(5):1714-24).

In conclusion, currently there are no satisfactory modes of therapy for the prevention and/or treatment of acute renal failure, and there is a need therefore to develop novel compounds for this purpose.

Glaucoma

Glaucoma is one of the leading causes of blindness in the world. It affects approximately 66.8 million people worldwide and at least 12,000 Americans are blinded by this disease each year (Kahn and Milton, Am J Epidemiol. 1980, 111(6):769-76). Glaucoma is characterized by the degeneration of axons in the optic nerve head, primarily due to elevated intraocular pressure (IOP). One of the most common forms of glaucoma, known as primary open-angle glaucoma (POAG), results from the increased resistance of aqueous humor outflow in the trabecular meshwork (TM), causing IOP elevation and eventual optic nerve damage.

Acute Respiratory Distress Syndrome

Acute respiratory distress syndrome (ARDS), also known as respiratory distress syndrome (RDS) or adult respiratory distress syndrome (in contrast with infant respiratory distress syndrome, IRDS) is a serious reaction to various forms of injuries to the lung. This is the most important disorder resulting in increased permeability pulmonary edema.

ARDS is a severe lung disease caused by a variety of direct and indirect insults. It is characterized by inflammation of the lung parenchyma leading to impaired gas exchange with concomitant systemic release of inflammatory mediators causing inflammation, hypoxemia and frequently resulting in multiple organ failure. This condition is life threatening, usually requiring mechanical ventilation and admission to an intensive care unit. A less severe form is called acute lung injury (ALI).

Spinal Cord Injury

Spinal cord injury or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter alia, and diseases which can affect the spinal cord include polio, spina bifida, tumors and Friedreich's ataxia.

Ischemia Reperfusion Injury Following Lung Transplantation

Lung transplantation, the only definitive therapy for many patients with end stage lung disease, has poor survival rates in all solid allograft recipients. Ischemia reperfusion (IR) injury is one of the leading causes of death in lung allograft recipients.

International patent application no. WO 2006/035434 assigned to the assignee of the present application discloses p53 inhibitors for the treatment of, inter alia, acute renal failure and hearing loss.

Diabetic Retinopathy

In the diabetic state, hyperglycemia leads to decreased retinal blood flow, retinal hyperpermeability, delays in photoreceptor nerve conduction, and retinal neuronal cell death. In short duration diabetes, neuronal cell death has been identified within the inner nuclear layer of the retina. Specifically, apoptosis has been localized to glial cells such as Mueller cells and astrocytes and has been shown to occur within 1 month of diabetes in the STZ-induced diabetic rat model. The cause of these events is multi-factorial including activation of the diacylglycerol/PKC pathway, oxidative stress, and nonenzymatic glycosylation. The combination of these events renders the retina hypoxic and ultimately leads to the development of diabetic retinopathy. One possible connection between retinal ischemia and the early changes in the diabetic retina is the hypoxia-induced production of growth factors such as VEGF. The master regulator of the hypoxic response has been identified as hypoxia inducible factor-1 (HIF-1), which controls genes that regulate cellular proliferation and angiogenesis. Prior studies have demonstrated that inhibition of HIF-1 ubiquitination leads to binding with hypoxia responsive elements (HRE) and production of VEGF mRNA.

Diabetic Retinopathy is defined as the progressive dysfunction of the retinal vasculature caused by chronic hyperglycemia. Key features of diabetic retinopathy include microaneurysms, retinal hemorrhages, retinal lipid exudates, cotton-wool spots, capillary nonperfusion, macular edema and neovascularization. Associated features include vitreous hemorrhage, retinal detachment, neovascular glaucoma, premature cataract and cranial nerve palsies.

A microvascular disease that primarily affects the capillaries, diabetes mellitus affects the eye by destroying the vasculature in the conjunctiva, retina and central nervous system.

Neuropathy

Neuropathy affects all peripheral nerves: pain fibers, motor neurons, autonomic nerves. It therefore necessarily can affect all organs and systems since all are innervated. There are several distinct syndromes based on the organ systems and members affected, but these are by no means exclusive. A patient can have sensorimotor and autonomic neuropathy or any other combination. Despite advances in the understanding of the metabolic causes of neuropathy, treatments aimed at interrupting these pathological processes have been limited by side effects and lack of efficacy. Thus, treatments are symptomatic and do not address the underlying problems. Agents for pain caused by sensorimotor neuropathy include tricyclic antidepressants (TCAs), serotonin reuptake inhibitors (SSRIs) and antiepileptic drugs (AEDs). None of these agents reverse the pathological processes leading to diabetic neuropathy and none alter the relentless course of the illness. Thus, it would be useful to have a pharmaceutical composition that could better treat these conditions and/or alleviate the symptoms.

Retinal Microvasculopathy (AIDS Retinopathy)

Retinal microvasculopathy is seen in 100% of AIDS patients. It is characterized by intraretinal hemorrhages, microaneurysms, Roth spots, cotton-wool spots (microinfarctions of the nerve fiber layer) and perivascular sheathing. The etiology of the retinopathy is unknown though it has been thought to be due to circulating immune complexes, local release of cytotoxic substances, abnormal hemorheology, and HIV infection of endothelial cells. AIDS retinopathy is now so common that cotton wool spots in a patient without diabetes or hypertension but at risk for HIV should prompt the physician to consider viral testing. There is no specific treatment for AIDS retinopathy but its continued presence may prompt a physician to reexamine the efficacy of the HIV therapy and patient compliance.

Bone Marrow Transplantation (BMT) Retinopathy

Bone marrow transplantation retinopathy was first reported in 1983. It typically occurs within six months, but it can occur as late as 62 months after BMT. Risk factors such as diabetes and hypertension may facilitate the development of BMT retinopathy by heightening the ischemic microvasculopathy. There is no known age, gender or race predilection for development of BMT retinopathy. Patients present with decreased visual acuity and/or visual field deficit. Posterior segment findings are typically bilateral and symmetric. Clinical manifestations include multiple cotton wool spots, telangiectasia, microaneurysms, macular edema, hard exudates and retinal hemorrhages. Fluorescein angiography demonstrates capillary nonperfusion and dropout, intraretinal microvascular abnormalities, microaneurysms and macular edema. Although the precise etiology of BMT retinopathy has not been elucidated, it appears to be affected by several factors: cyclosporine toxicity, total body irradiation (TBI), and chemotherapeutic agents. Cyclosporine is a powerful immunomodulatory agent that suppresses graft-versus-host immune response. It may lead to endothelial cell injury and neurological side effects, and as a result, it has been suggested as the cause of BMT retinopathy. However, BMT retinopathy can develop in the absence of cyclosporine use, and cyclosporine has not been shown to cause BMT retinopathy in autologous or syngeneic bone marrow recipients. Cyclosporine does not, therefore, appear to be the sole cause of BMT retinopathy. Total body irradiation (TBI) has also been implicated as the cause of BMT retinopathy. Radiation injures the retinal microvasculature and leads to ischemic vasculopathy. Variables such as the total dose of radiation and the time interval between radiation and bone marrow ablation appear to be important. However, BMT retinopathy can occur in patients who did not receive TBI, and BMT retinopathy is not observed in solid organ transplant recipients who received similar doses of radiation. Thus, TBI is not the sole cause, but it is another contributing factor in development of BMT retinopathy. Chemotherapeutic agents have been suggested as a potential contributing factor in BMT retinopathy. Medications such as cisplatin, carmustine, and cyclophosphamide can cause ocular side effects including papilledema, optic neuritis, visual field deficit and cortical blindness. It has been suggested that these chemotherapeutic drugs may predispose patients to radiation-induced retinal damages and enhance the deleterious effect of radiation. In general, patients with BMT retinopathy have a good prognosis. The retinopathy usually resolves within two to four months after stopping or lowering the dosage of cyclosporine. In one report, 69 percent of patients experienced complete resolution of the retinal findings, and 46 percent of patients fully recovered their baseline visual acuity. Because of the favorable prognosis and relatively non-progressive nature of BMT retinopathy, aggressive intervention is usually not necessary.

Microvascular Diseases of the Kidney

The kidney is involved in a number of discreet clinicopathologic conditions that affect systemic and renal microvasculature. Certain of these conditions are characterized by primary injury to endothelial cells, such as: Hemolytic-uremic syndrome (HUS) and thrombotic thrombocytopenic purpura (TTP) and Radiation nephritis—The long-term consequences of renal irradiation in excess of 2500 rad.

In other kidney diseases, the microvasculature of the kidney is involved in autoimmune disorders, such as systemic sclerosis (scleroderma). Kidney involvement in systemic sclerosis manifests as a slowly progressing chronic renal disease or as scleroderma renal crisis (SRC), which is characterized by malignant hypertension and acute azotemia. It is postulated that SRC is caused by a Raynaud-like phenomenon in the kidney. Severe vasospasm leads to cortical ischemia and enhanced production of renin and angiotensin II, which in turn perpetuate renal vasoconstriction. Hormonal changes (pregnancy), physical and emotional stress, or cold temperature may trigger the Raynaud-like arterial vasospasm. The role of the renin-angiotensin system in perpetuating renal ischemia is underscored by the significant benefit of ACE inhibitors in treating SRC. In patients with SRC who progress to severe renal insufficiency despite antihypertensive treatment, dialysis becomes a necessity. Both peritoneal dialysis and hemodialysis have been employed. The End-Stage Renal Disease (ESRD) Network report on 311 patients with systemic sclerosis-induced ESRD dialyzed between 1983 and 1985 revealed a 33% survival rate at 3 years.

The renal microcirculation can also be affected in sickle cell disease, to which the kidney is particularly susceptible because of the low oxygen tension attained in the deep vessels of the renal medulla as a result of countercurrent transfer of oxygen along the vasa recta. The smaller renal arteries and arterioles can also be the site of thromboembolic injury from cholesterol-containing material dislodged from the walls of the large vessels.

Taken as a group, diseases that cause transient or permanent occlusion of renal microvasculature uniformly result in disruption of glomerular perfusion, and hence of the glomerular filtration rate, thereby constituting a serious threat to systemic homeostasis.

Oral Mucositis

Oral mucositis, also referred to as stomatitis, is a common and debilitating side effect of chemotherapy and radiotherapy regimens, which manifests itself as erythema and painful ulcerative lesions of the mouth and throat. Routine activities such as eating, drinking, swallowing, and talking may be difficult or impossible for subjects with severe oral mucositis. Palliative therapy includes administration of analgesics and topical rinses.

Dry-Eye Syndrome

Dry eye syndrome is a common problem usually resulting from a decrease in the production of tear film that lubricates the eyes. Most patients with dry eye experience discomfort, and no vision loss; although in severe cases, the cornea may become damaged or infected. Wetting drops (artificial tears) may be used for treatment while lubricating ointments may help more severe cases.

Ocular Ischemic Conditions

Ischemic optic neuropathy (ION) includes a variety of disorders that produce ischemia to the optic nerve. By definition, ION is termed anterior if disc edema is present acutely, suggesting infarction of the portion of the optic nerve closest to the globe. ION also may be posterior, lying several centimeters behind the globe. Ischemic optic neuropathy usually occurs only in people older than 60 years of age. Most cases are nonarteritic and attributed to the effects of atherosclerosis, diabetes, or hypertension on optic nerve perfusion. Temporal arteritis causes about 5% of cases (arteritic ION).

Ischemic Optic Neuropathy (ION)

A severely blinding disease resulting from loss of the arterial blood supply to the optic nerve (usually in one eye), as a result of occlusive disorders of the nutrient arteries. Optic neuropathy can be anterior (AION), which causes a pale edema of the optic disc, or posterior, in which the optic disc is not swollen and the abnormality occurs between the eyeball and the optic chiasm. Ischemic anterior optic neuropathy usually causes a loss of vision that may be sudden or occur over several days. Ischemic posterior optic neuropathy is uncommon, and the diagnosis depends largely upon exclusion of other causes, chiefly stroke and brain tumor.

More effective therapies to treat the above mentioned diseases and disorders would be of great therapeutic value.

Organ Transplantation

In various embodiments the chemically modified siRNA compounds of the application are useful for treating or preventing injury, including reperfusion injury, following organ transplantation including lung, liver, heart, bone pancreas, intestine, skin, blood vessels, heart valve, bone and kidney transplantation.

The term “organ transplant” is meant to encompass transplant of any one or more of the following organs including, inter alia, lung, kidney, heart, skin, vein, bone, cartilage, liver transplantation. Although a xenotransplant can be contemplated in certain situations, an allotransplant is usually preferable. An autograft can be considered for bone marrow, skin, bone, cartilage and or blood vessel transplantation.

The siRNA compounds of the present application are particularly useful in treating a subject experiencing the adverse effects of organ transplant, including ameliorating, treating or preventing perfusion injury.

For organ transplantation, either the donor or the recipient or both may be treated with a chemically modified siRNA compound of the present application or pharmaceutical composition comprising at least one of the siRNA compounds of the application. Accordingly, the present application relates to a method of treating an organ donor or an organ recipient comprising the step of administering to the organ donor or organ recipient or both a therapeutically effective amount of at least one chemically modified siRNA compound according to the present application.

The application further relates to a method for preserving an organ comprising contacting the organ with an effective amount of at least one siRNA compound of the present application. Also provided is a method for reducing or preventing injury (in particular reperfusion injury) of an organ during surgery and/or following removal of the organ from a subject comprising placing the organ in an organ preserving solution wherein the solution comprises at least one chemically modified siRNA compound according to the present application.

Lung Transplantation

Indications for lung transplantation include chronic obstructive pulmonary disease (COPD), pulmonary hypertension, cystic fibrosis, idiopathic pulmonary fibrosis, and Eisenmenger syndrome. Typically, four different surgical techniques are used: single-lung transplantation, bilateral sequential transplantation, combined heart-lung transplantation, and lobar transplantation, with the majority of organs obtained from deceased donors.

The medical complications associated with lung transplantation include surgical complications, graft rejection, or immunosuppression.

Graft rejection has been categorized into three subcategories (i) hyperacute rejection is the term applied to very early graft destruction, usually within the first 48-72 hours; (ii) acute rejection has an onset of several days to months or even years after transplantation and can involve humoral and/or cellular mechanisms; (iii) Chronic rejection relates to chronic alloreactive immune response.

Hyperacute, or primary, graft failure occurs within 72 hours postoperatively resulting from ischemia-reperfusion (IR) injury and presents similarly to acute respiratory distress syndrome (ARDS). Mortality may reach up to 60%, and patients who survive may have a prolonged recovery period with significant pulmonary function impairments.

Acute graft rejection typically occurs in the first 3 months post transplantation. Acute graft rejection is characterized by a host T-cell response toward the transplanted organ. Clinical features of acute graft rejection are nonspecific and include one or more of dyspnea, fever, leukocytosis, nonproductive cough, hypoxemia, and malaise. Acute allograft rejection remains a significant problem in lung transplantation despite advances in immunosuppressive medication. Rejection, and ultimately early morbidity and mortality may result from ischemia-reperfusion (I/R) injury and hypoxic injury.

The clinical course is variable and depends on the severity of rejection; mild cases of rejection may even be asymptomatic. Treatment for acute graft rejection is high-dose parenteral steroids.

In mild chronic rejection, the patient may present with a nonproductive cough and dyspnea on exertion, that can progress to dyspnea at rest, productive cough, pseudomonas colonization, and chest radiographic findings of bronchiectasis and air trapping. Histologic changes involve either the vasculature or the airways. Chronic vascular rejection is caused by atherosclerosis of the pulmonary vasculature, while chronic airway rejection is caused by bronchiolitis obliterans.

In some embodiments the target gene is selected from P53, NOX2 and NOX4. In yet other embodiments the sense and antisense oligonucleotide sequences useful in synthesizing siRNA compounds are set forth in Tables B1, A5, A1 and A2.

Pharmaceutical Compositions

The present application provides a pharmaceutical composition comprising one or more of the compounds of the application; and a pharmaceutically acceptable carrier. In various embodiments such compositions comprise a mixture of two or more different oligonucleotides/siRNAs.

The application further provides a pharmaceutical composition comprising at least one compound of the application covalently or non-covalently bound to one or more compounds of the application in an amount effective to down-regulate target gene expression or activity; and a pharmaceutically acceptable carrier. Endogenous cellular complexes to produce one or more oligoribonucleotides of the application may process the compound intracellularly.

The present application also provides for a process of preparing a pharmaceutical composition, which comprises:

providing one or more siRNA compounds of the application; and

admixing said compound with a pharmaceutically acceptable carrier.

Substantially complementary refers to complementarity of greater than about 84%, to another sequence. For example in a duplex region consisting of 19 base pairs one mismatch results in 94.7% complementarity, two mismatches results in about 89.5% complementarity and 3 mismatches results in about 84.2% complementarity, rendering the duplex region substantially complementary. Accordingly substantially identical refers to identity of greater than about 84%, to another sequence.

Additionally, the application provides a method of inhibiting the expression of the genes of the present application by at least 50% as compared to a control comprising contacting an mRNA transcript of the gene of the present application with one or more of the compounds of the application.

In one embodiment the oligoribonucleotide is inhibiting a target gene, whereby the inhibition is selected from the group comprising inhibition of gene function, inhibition of polypeptide and inhibition of mRNA expression. In various embodiments target gene is selected from NOX4, NOX1, NOX2 (gp91phox, CYBB), NOX5, DUOX2, NOXO1, NOXO2 (NCF1), NOXA1, NOXA2 (p67phox, NCF2), TP53; HTRA2; KEAP1; SHC1, ZNHIT1, LGALS3, and HI95.

In additional embodiments the application provides a method of treating a subject at risk of or suffering from, a disease accompanied by an elevated level of one or more of the target genes/polypeptides, the method comprising administering to the subject a compound of the application in a therapeutically effective dose thereby treating the subject.

More particularly, the application provides a chemically modified double stranded oligoribonucleotide wherein one strand comprises consecutive nucleotides having, from 5′ to 3′, the sequence set forth in any one of Tables A1-A18 or Tables B1-B15, SEQ ID NOS:24-23,157, or a homolog thereof wherein in up to two of the ribonucleotides in each terminal region is altered.

Delivery

The siRNA molecules of the present application may be delivered to the target tissue by direct application of the naked molecules prepared with a carrier or a diluent.

The term “naked siRNA” refers to siRNA molecules that are free from any delivery vehicle that acts to assist, promote or facilitate entry into the cell, including viral sequences, viral particles, liposome formulations, lipofectin or precipitating agents and the like. For example, siRNA in PBS is “naked siRNA”.

However, in some embodiments the siRNA molecules of the application are delivered in liposome formulations and lipofectin formulations and the like and can be prepared by methods well known to those skilled in the art. Such methods are described, for example, in U.S. Pat. Nos. 5,593,972, 5,589,466, and 5,580,859, which are herein incorporated by reference.

For delivery of siRNAs, see, for example, Larson, S D et al., Surgery 2007. 142:262-69; Shen et al., 2003. FEBS Letters 539: 111-114; Xia et al., 2002. Nat Biotech 20: 1006-1010, Reich et al., 2003. Molec. Vision 9: 210-216, Sorensen et al. 2003. J. Mol. Biol. 327: 761-766, Lewis et al., 2002. Nat Genet 32: 107-108; Simeoni et al., 2003. NAR 31, 11: 2717-2724. International Patent Application Publication No. WO 2007/107789 relates to intranasal delivery of siRNA to the CNS.

The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the application and they include liposomes and microspheres. Examples of delivery systems useful in the present application include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art. In one specific embodiment of this application topical and transdermal formulations may be selected. The siRNAs or pharmaceutical compositions of the present application are administered and dosed in accordance with good medical practice, taking into account the clinical condition of the individual patient, the disease to be treated, the site and method of administration, scheduling of administration, patient age, sex, body weight and other factors known to medical practitioners.

The “therapeutically effective dose” for purposes herein is thus determined by such considerations as are known in the art. The dose must be effective to achieve improvement including but not limited to improved survival rate or more rapid recovery, or improvement or elimination of symptoms and other indicators as are selected as appropriate measures by those skilled in the art.

In general, the active dose of compound for humans is in the range of from 1 ng/kg to about 20-100 mg/kg body weight per day, preferably about 0.01 mg to about 2-10 mg/kg body weight per day, in a regimen of a single does, one dose per day or twice or three or more times per day for 1 day or for several days or for a period of 1-4 weeks or longer.

The compounds of the present application can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal, inhalation, transtympanic administration as well as intrathecal and infusion techniques. Implants of the compounds are also useful. Liquid forms may be prepared for injection, the term including subcutaneous, transdermal, intravenous, intramuscular, intrathecal, intranasal and other parental routes of administration. The liquid compositions include aqueous solutions, with and without organic co-solvents, aqueous or oil suspensions, emulsions with edible oils, as well as similar pharmaceutical vehicles. In a particular embodiment, the administration comprises intravenous administration. In another embodiment the administration comprises topical or local administration. In a non-limiting example, siRNA that targets NOX2 or NOXO1 is useful in treating a subject suffering from a neurodegenerative disease (AD, ALS) and the siRNA is delivered to the CNS by intranasal administration.

In addition, in certain embodiments the compositions for use in the novel treatments of the present application may be formed as aerosols, for example for intranasal administration.

In certain embodiments, oral compositions (such as tablets, suspensions, solutions) may be effective for local delivery to the oral cavity such as oral composition suitable for mouthwash for the treatment of oral mucositis.

The compounds of the present application can be administered topically to the surface of the eye. It should be noted that the compound is preferably administered as the compound or as pharmaceutically acceptable salt active ingredient in combination with pharmaceutically acceptable carriers, solvents, diluents, excipients, adjuvants and or vehicles. According to the present application the preferred method of delivery is topical administration for topical delivery to the eye.

Liquid forms are prepared for drops or spray. The liquid compositions include aqueous solutions, with and without organic co-solvents, aqueous or oil suspensions, emulsions with oils, as well as similar pharmaceutical vehicles. In some embodiments administration comprises topical or local administration.

These compounds are administered to humans and other animals for therapy by any suitable route of administration to the eye, as by, for example, a spray or drops, and topically, as by ointments, suspensions or drops.

In preferred embodiments the subject being treated is a warm-blooded animal and, in particular, mammals including human.

Further, the present application provides for a pharmaceutical composition comprising any one of the above compounds and a pharmaceutically acceptable excipient.

Suitable methods for delivery of the siRNA comprise, among others, transfection, lipofection, electroporation and infection with recombinant viral vectors. In connection with the present application, an additional feature of the vector is in one embodiment an expression limiting feature such as a promoter and regulatory element, respectively, that are specific for the desired cell type thus allowing the expression of the nucleic acid sequence according to the present application only once the background is provided which allows the desired expression.

In a further aspect the present application is related to a pharmaceutical composition comprising a nucleic acid according to the present application and/or a vector according to the present application and, optionally, a pharmaceutically acceptable carrier, diluent or adjuvants or other vehicle(s). Preferably, such carrier, diluents, adjuvants and vehicles are inert, and non-toxic. The pharmaceutical composition is in its various embodiments adapted for administration in various ways. Such administration comprises systemic and local administration as well as oral, subcutaneous, parenteral, intravenous, intraarterial, intramuscular, intraperitonial, intranasal, and intrategral.

In particular embodiments the siRNA compound is formulated as eye drops for administration to the surface of the eye. In other embodiments the siRNA compound is administered to the lung by inhalation. In yet other embodiments the siRNA compound is formulated for delivery to the inner ear by transtympanic injection or via eardrops.

It will be acknowledged by the one skilled in the art that the amount of the pharmaceutical composition and the respective siRNA depends on the clinical condition of the individual patient, the site and method of administration, scheduling of administration, patient age, sex, bodyweight and other factors known to medical practitioners. The pharmaceutically effective amount for purposes of prevention and/or treatment is thus determined by such considerations as are known in the medical arts. Preferably, the amount is effective to achieve improvement including but limited to improve the diseased condition or to provide for a more rapid recovery, improvement or elimination of symptoms and other indicators as are selected as appropriate measures by those skilled in the medical arts.

In a preferred embodiment, the pharmaceutical composition according to the present application may comprise other pharmaceutically active compounds. Preferably, such other pharmaceutically active compounds are selected from the group comprising compounds which allow for uptake intracellular cell delivery, compounds which allow for endosomal release, compounds which allow for, longer circulation time and compounds which allow for targeting of endothelial cells or pathogenic cells.

The pharmaceutical composition is preferably formulated so as to provide for a single dosage administration or a multi-dosage administration.

The pharmaceutical composition according to the present application can also be used in a method for preventing and/or treating a disease as disclosed herein, whereby the method comprises the administration of a nucleic acid according to the present application, a vector according to the present application or a pharmaceutical composition or medicament according to the present application for any of the diseases described herein.

The synthesis of any of the nucleic acids described herein is within the skills of the one of the art. Such synthesis is, among others, described in Beaucage S L and Iyer R P, 1992 Tetrahedron; 48: 2223-2311, Beaucage S. and Iyer R P, 1993 Tetrahedron; 49: 6123-6194 and Caruthers M H et. al., 1987 Methods Enzymol.; 154: 287-313, the synthesis of thioates is, among others, described in Eckstein F., 1985 Annu Rev. Biochem.; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

siRNA for any one of the target genes are synthesized using methods known in the art as described above, based on the known sequence of the target mRNA (SEQ ID NOS:1-23), and can be made stable by various modifications as described above.

An additional aspect of the present application provides for methods of treating an apoptosis related disease. Methods for therapy of diseases or disorders associated with uncontrolled, pathological cell growth, e.g. cancer, psoriasis, autoimmune diseases, inter alia, and methods for therapy of diseases associated with ischemia and lack of proper blood flow, e.g. myocardial infarction (MI) and stroke, are provided. “Cancer” or “Tumor” refers to an uncontrolled growing mass of abnormal cells. These terms include both primary tumors, which may be benign or malignant, as well as secondary tumors, or metastases which have spread to other sites in the body. Examples of cancer-type diseases include, inter alia: carcinoma (e.g.: breast, colon and lung), leukemia such as B cell leukemia, lymphoma such as B-cell lymphoma, blastoma such as neuroblastoma and melanoma and sarcoma. It will be acknowledged that the pharmaceutical composition according to the present application can be used for any disease which involves undesired development or growth of vasculature including angiogenesis, as well as any of the diseases and conditions described herein. Preferably, these kind of diseases are tumor diseases. Among tumor diseases, the following tumors are most preferred: endometrial cancer, colorectal carcinomas, gliomas, endometrial cancers, adenocarcinomas, endometrial hyperplasias, Cowden's syndrome, hereditary non-polyposis colorectal carcinoma, Li-Fraumene's syndrome, breast-ovarian cancer, prostate cancer (Ali, I. U., Journal of the National Cancer Institute, Vol. 92, no. 11, Jun. 7, 2000, page 861-863), Bannayan-Zonana syndrome, LDD (Lhermitte-Duklos' syndrome) (Macleod, K., supra) hamartoma-macrocephaly diseases including Cow disease (CD) and Bannayan-Ruvalcaba-Rily syndrome (BRR), mucocutaneous lesions (e. g. trichilemmonmas), macrocephaly, mental retardation, gastrointestinal harmatomas, lipomas, thyroid adenomas, fibrocystic disease of the breast, cerebellar dysplastic gangliocytoma and breast and thyroid malignancies (Vazquez, F., Sellers, W. R., supra).

Combination Therapy

The present application provides for combination therapy for all the conditions disclosed herein and in particular ischemic and ND conditions. In said combination therapy, one or more of the target genes are targeted to ameliorate symptoms of the disease being treated. These genes are inhibited with a combination of siRNAs or antibodies (including aptamer antibodies) or both.

This application also comprises a tandem double-stranded structure which comprises two or more siRNA sequences, which is processed intracellularly to form two or more different siRNAs, one inhibiting one target gene and a second inhibiting another target gene. In a related aspect, this application also comprises a tandem double-stranded structure which comprises two or more siRNA sequences, which is degraded intracellularly to form two or more different siRNAs, both inhibiting the same target gene.

In particular, it is envisaged that a long oligonucleotide (typically about 80-500 nucleotides in length) comprising one or more stem and loop structures, where stem regions comprise the sequences of the oligonucleotides of the application, are delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the application. This oligonucleotide can be termed a tandem shRNA construct. It is envisaged that this long oligonucleotide is a single stranded oligonucleotide comprising one or more stem and loop structures, wherein each stem region comprises a sense and corresponding antisense siRNA sequence of one or more of the target genes. In particular, it is envisaged that this oligonucleotide comprises sense and antisense siRNA oligonucleotide pairs as depicted in any one of Tables A1-A18, B1-B15 or C1-C2. Alternatively, the tandem construct may comprise sense and complementary antisense siRNA sequence corresponding to a target gene.

The application has been described in an illustrative manner, and it is to be understood that the terminology which has been used is intended to be in the nature of words of description rather than of limitation.

Obviously, many modifications and variations of the present application are possible in light of the above teachings. It is, therefore, to be understood that within the scope of the appended claims, the application can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this application pertains.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

The present application is illustrated in detail below with reference to examples, but is not to be construed as being limited thereto.

### EXAMPLES

Without further elaboration, it is believed that one skilled in the art can, using the preceding description, utilize the present application to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic Syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th ed. (2001).

Standard medicinal chemistry methods known in the art not specifically described herein are generally followed essentially as in the series “Comprehensive Medicinal Chemistry”, by various authors and editors, published by Pergamon Press.

The features of the present application disclosed in the specification, the claims and/or the drawings may both separately and in any combination thereof be material for realizing the invention in various forms thereof.

General Materials and Methods

Cell Culture

The first human cell line, namely HeLa cells (American Type Culture Collection) were cultured as follows: HeLa cells (American Type Culture Collection) were cultured as described in Czauderna F et al. (Czauderna, F., et al., 2003. NAR, 31, 670-82).

The second human cell line was a human keratinocyte cell line which was cultivated as follows: Human keratinocytes were cultured at 37° C. In Dulbecco's modified Eagle medium (DMEM) containing 10% FCS. The mouse cell line was B16V (American Type Culture Collection) cultured at 37° C. In Dulbecco's modified Eagle medium (DMEM) containing 10% FCS. Culture conditions were as described in Methods Find Exp Clin Pharmacol. 1997 19(4):231-9.

In each case, the cells were subject to the experiments as described herein at a density of about 50,000 cells per well and the double-stranded nucleic acid according to the present application was added at 20 nM, whereby the double-stranded nucleic acid was complexed using 1 μg/ml of a proprietary lipid.

Induction of Hypoxia-Like Condition

The cells are treated with CoCl₂ for inducing a hypoxia-like condition as follows: siRNA transfections are carried out in 10-cm plates (30-50% confluency) as described by (Czauderna et al., 2003; Kretschmer et al., 2003). Briefly, siRNA are transfected by adding a preformed 10× concentrated complex of GB and lipid in serum-free medium to cells in complete medium. The total transfection volume is 10 ml. The final lipid concentration is 1.0 μg/ml; the final siRNA concentration is 20 nM unless otherwise stated. Induction of the hypoxic responses is carried out by adding CoCl₂ (100 μM) directly to the tissue culture medium 24 h before lysis.

Preparation of Cell Extracts and Immuno Blotting

The preparation of cell extracts and immuno blot analysis were carried out essentially as described by Klippel et al. (Klippel, A., et al., 1998. Mol Cell Biol, 18, 5699-711; Klippel, A., et al., 1996. Mol Cell Biol, 16, 4117-27). The murine monoclonal anti-p110a and anti-p85 antibodies have been described by Klippel et al. (supra).

In Vitro Testing of siRNA Compounds

About 1.5-2×10⁵ tested cells (HeLa cells and/or 293T cells for siRNA targeting human genes and NRK52 (normal rat kidney proximal tubule cells) cells and/or NMuMG cells (mouse mammary epithelial cell line) for siRNA targeting the rat/mouse gene) were seeded per well in 6 wells plate (70-80% confluent). See also Examples hereinbelow.

About 24 hours later, cells were transfected with siRNA compounds using the Lipofectamine™ 2000 reagent (Invitrogen) at final concentrations of 5 nM or 20 nM. The cells were incubated at 37° C. In a CO₂ incubator for 72 h.

As positive control for transfection PTEN-Cy3 labeled siRNA compounds were used. Various chemically modified blunt ended siRNA compounds having alternating modified and unmodified ribonucleotides (modified at the 2′ position of the sugar residue in both the antisense and the sense strands, wherein the moiety at the 2′ position of the sugar is methoxy) and wherein the ribonucleotides at the 5′ and 3′ termini of the antisense strand are modified in their sugar residues, and the ribonucleotides at the 5′ and 3′ termini of the sense strand are unmodified in their sugar residues were tested. Another siRNA compound comprised a blunt ended structure having an antisense with an alternating pattern of methoxy moieties and a sense strand with three ribonucleotides linked by two 2′5′ bridges at the 3′ terminus; and another siRNA compound comprising antisense and sense strands having three ribonucleotides linked by 2′5′ bridges at the 3′ terminus was used. Some of the tested compounds comprised a blunt ended structure having an antisense with an alternating pattern of methoxy moieties and a sense strand with one or two L-deoxyribonucleotides at the 3′ terminal or 3′ penultimate positions.

GFP and or scrambled siRNA compounds were used as negative control for siRNA activity.

At 72 h after transfection cells were harvested and RNA was extracted from cells. Transfection efficiency was tested by fluorescent microscopy.

The percent of inhibition of gene expression using specific preferred siRNA structures was determined using qPCR analysis of a target gene in cells expressing the endogenous gene.

In general, the siRNAs having specific sequences that were selected for in vitro testing were specific for human and a second species such as non-human primate, rat or rabbit genes. Similar results are obtained using siRNAs having these RNA sequences and modified as described herein.

Serum Stability Experiments

Chemically modified siRNA compounds according to the present application were tested for duplex stability in human serum, as follows:

siRNA molecules at final concentration of 7 uM were incubated at 37° C. In 100% human serum (Sigma Cat #H4522). (siRNA stock 100 uM diluted in human serum 1:14.29).

5 ul were added to 15 ul 1.5XTBE-loading buffer at different time points (0, 30 min, 1 h, 3 h, 6 h, 8 h, 10 h, 16 h and 24 h)

Samples were immediately frozen in liquid nitrogen and were kept at −20° C.

Each sample was loaded onto a non-denaturing 20% acrylamide gel, prepared according to methods known in the art.

The oligos were visualized with Ethidium bromide under UV light.

### Example 1

### Selection and Preparation of siRNAs

Using proprietary algorithms and the known sequence of the mRNA of the target genes mRNA polynucleotides (SEQ ID NO:1-23), the sequences of many potential siRNAs were generated. siRNA molecules according to the above specifications were prepared essentially as described herein.

The siRNAs of the present application can be synthesized by any of the methods which are well-known in the art for synthesis of ribonucleic (or deoxyribonucleic) oligonucleotides. For example, a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present application, two or more such sequences can be synthesized and linked together for use in the present application.

The siRNA molecules of the application are synthesized by procedures known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and may make use of common nucleic acid protecting and coupling groups, such as dimethoxytrityl at the 5′-end, and phosphoramidites at the 3′-end. The modified (e.g.—DNA, 2′-5′, 2′-O-methylated)nucleotides and unmodified nucleotides are incorporated as desired.

Alternatively, the nucleic acid molecules of the present application can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siRNA molecules of the application can also be synthesized via a tandem synthesis methodology, as described in US patent application publication No. US2004/0019001 (McSwiggen) wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

Sense and antisense sequences presented in Tables A1-A18, B1-B15 and C1-C2 are depicted in the 5′ to 3′ orientation.

### Example 2

### Chemically Modified siRNA Compounds

Table 2 hereinbelow provides a code of the modified nucleotides/unconventional moieties utilized in preparing the siRNA oligonucleotides of the present application.

TABLE 2

| Code | modification |
|---|---|
| Nuc | |
| 5medG | 5-methyl-deoxyriboguanosine-3′-phosphate |
| c6Np | Amino modifier C6 (Glen Research 10-1906-xx) |
| dA | deoxyriboadenosine-3′-phosphate |
| dB | abasic deoxyribose-3′-phosphate |
| dC | deoxyribocytidine-3′-phosphate |
| dG | deoxyriboguanosine-3′-phosphate |
| dT | thymidine-3′-phosphate |
| dT$ | thymidine (no phosphate) |
| enaA$ | ethylene-bridged nucleic acid adenosine (no phosphate) |
| enaC | ethylene-bridged nucleic acid cytidine 3′ phosphate |
| enaG | ethylene-bridged nucleic acid guanosine 3′ phosphate |
| enaT | ethylene-bridged nucleic acid thymidine 3′ phosphate |
| iB | inverted deoxyabasic |
| LdA | L-deoxyriboadenosine-3′-phosphate (mirror image dA) |
| LdA$ | L-deoxyriboadenosine (no phosphate) (mirror image dA) |
| LdC | L-deoxyribocytidine-3′-phosphate (mirror image dC) |
| LdC$ | L-deoxyribocytidine (no phosphate) (mirror image dC) |
| LdG | L-deoxyriboguanosine-3′-phosphate (mirror image dG) |
| LdT | L-deoxyribothymidine-3′-phosphate (mirror image dT) |
| LdT$ | L-deoxyribothymidine (no phosphate) (mirror image dT) |
| mA | 2′-O-methyladenosine-3′-phosphate |
| mA$ | 2′-O-methyladenosine (no phosphate) |
| mC | 2′-O-methylcytidine-3′-phosphate |
| mC$ | 2′-O-methylcytidine (no 3′-phosphate) |
| mG | 2′-O-methylguanosine-3′-phosphate |
| mG$ | 2′-O-methylguanosine (no phosphate) |
| mU | 2′-O-methyluridine-3′-phosphate |
| mU$ | 2′-O-methyluridine (no phosphate) |
| rA | riboadenosine-3′-phosphate |
| rA$ | riboadenosine (no phosphate) |
| rC | ribocytidine-3′-phosphate |
| rC$ | ribocytidine (no phosphate) |
| rC2p | ribocytidine-2′-phosphate |
| rG | riboguanosine-3′-phosphate |
| rG2p | riboguanosine-2′-phosphate |
| rU | ribouridine-3′-phosphate |
| rU$ | ribouridine (no phosphate) |
| rU2p | ribouridine-2′-phosphate |

Tables A1-A18, B1-B15 and C1-C2 provide antisense and sense oligonucleotide pairs useful in synthesis of the siRNA compounds according to the present application. All oligonucleotides are presented as 5′-3′ sequences. (Hum=human, chn or chl=chinchilla, chimp or chmp=chimpanzee, ms=mouse, GP=guinea-pig; Rb=rabbit, MnkK or MK=monkey).

This application contains nucleotide sequences which are filed in electronic form via EFS-web. The file [199-PCT1_ST25.txt] was prepared using PatentIn v3.3 on Dec. 17, 2009. The sequences are also shown in Tables A1-A18 and B1-B15 infra, following the Examples. The entire content of this file is incorporated by reference into this application.

Certain preferred sense and antisense oligonucleotide pairs are shown in Tables C1 and C2 herein below.

TABLE C1

| siRNA | Sense (5′ > 3′) | Antisense (5′ > 3′) |
|---|---|---|
| NOX4_55 | CAGAAGAUAAUUGGAGAAA | UUUCUCCAAUUAUCUUCUG |
| NOX4_1 | CCAAGACTCTTCATAATTT | AAATTATGAAGAGTCTTGG |
| NOX4_21 | GGAACTCCGTATGATCAAA | TTTGATCATACGGAGTTCC |
| NOX4_22 | CCGTTTGCATCGATACTAA | TTAGTATCGATGCAAACGG |
| NOX4_25 | GCCCTTCATTCACTCTAGA | TCTAGAGTGAATGAAGGGC |
| NOX4_a | AUGCAAUUUCUAAGACUU | AAGUCUUAGAAAUUGCACU |
| CYBB_a | CCAACUGGGAUAAUGAAUU | AAUUCAUUAUCCCAGUUGG |
| CYBB_b | GGGUCUCAUAUUGGUCAUU | AAUGACCAAUAUGAGACCC |
| CYBB_c | CCAGAUCAUUAGGACAUAA | UUAUGUCCUAAUGAUCUGG |
| CYBB_d | GGGUUUUGUUUUUGUUCUU | AAGAACAAAAAGAAAACCC |
| NOX1_a | GGCUCAAUGUUUUCCUGUU | AACAGGAAAACAUUCAGCC |
| NOX1_b | CUCUCUGCUUGAAUUUUAA | UUAAAAUUCAAGCAGAGAG |
| NOX1_c | CCGACAAAUACUACUACAC | GUGUAGUAGUAUUUGUCGG |
| NOX1_d | CUCCAGCCUAUCUCAUGAU | AUCAUGAGAUAGGCUGGAG |
| NOX1_e | CACAUCUGUUUAACUUUGA | UCAAAGUUAAACAGGUGUG |
| NOXO1_a | CAGAUCAAGAGGCUCCAAA | UUUGGAGCCUCUUGAUCUG |
| NOXO1_b | GACUGUUGGAAACGUCAGA | UCUGACGUUUCCAACACGC |
| NOXO1_c | GCUUGUUGGAAACCUAUUCU | AGAAUAGGUUUCCAACAGC |
| NOXO1_d | GGAGUUGGGACGAAUUCAG | CUGAAUUCGUCCCAACUCC |

TABLE C2

| siRNA | Sense (5′ > 3′) | Antisense (5′ > 3′) |
|---|---|---|
| TP53-I5 | GAGAAUAUUUCACCCUUCA | UGAAGGGUGAAAUAUUCUC |
| TP53-F3 | CCGAGUGGAAGGAAAUUUG | CAAAUUUCCUUCCACUCGG |
| TP53-G1 | GACAGAAACACUUUUCGAC | GUCGAAAAGUGUUUCUGUC |
| HTRA2_11 | GAAUCACAGAAACACUUUU | AAAAGUGUUUCUGUGAUUC |
| HTRA2_12 | GGCCUGGUGAUGUGAUUUU | AAAAUCACAUCACCAGGCC |
| HTRA2_13 | CCGUGGUCUAUAUCGAGAU | AUCUCGAUAUAGACCACGG |
| HTRA2_15 | CCUAGCAACAUAUUAUAGU | ACUAUAAUAUGUUGCUAGG |
| HTRA2_16 | GCCGUGGUCUAUAUCGAGA | UCUCGAUAUAGACCACGGC |
| HTRA2_17 | GUGCUGCUCUUUGUGGUGU | ACACCACAAAGAGCAGCAC |
| HTRA2_18 | CAGCUAUUGAUUUUGGAAA | UUUCCAAAAUCAAUAGCUG |
| HTRA2_21 | GCUAUUGAUUUUGGAAACU | AGUUUCCAAAAUCAAUAGC |
| HTRA2_22 | AGCUAUUGAUUUUGGAAAC | GUUUCCAAAAUCAAUAGCU |
| KEAP1_2 | GCCUCAUUGAAUUCGCCUA | UAGGCGAAUUCAAUGAGGC |
| KEAP1_11 | CACCAUGUGAUUUAUUCUU | AAGAAUAAAUCACAUGGUG |
| KEAP1_12 | ACUGCAAAUAACCCAUCUU | AAGAUGGGUUAUUUGCAGU |
| KEAP1_13 | CACUGCAAAUAACCCAUCU | AGAUGGGUUAUUUGCAGUG |
| KEAP1_14 | GCAGCUGUCACCAUGUGAU | AUCACAUGGUGACAGCUGC |
| KEAP1_17 | UGCAUCAACUGGGUCAAGU | ACUUGACCCAGUUGAUGCA |
| SHC1_1 | ACCUGAAAUUUGCUGGAAU | AUUCCAGCAAAUUUCAGGU |
| SHC1_2 | CAGAGAGCUUUUUGAUGAU | AUCAUCAAAAAGCUCUCUG |
| SHC1_3 | CACAUGCAAUCUAUCUCAU | AUGAGAUAGAUUGCAUGUG |
| SHC1_6 | CGGGAGCUUUGUCAAUAAG | CUUAUUGACAAAGCUCCCG |
| SHC1_8 | GGGUUCUUAUAAUGGAAAA | UUUUCCAUUAUAAGAACCC |
| SHC1_11 | CCCAAGCCCAAGUACAAUC | GAUUGUACUUGGGCUUGGG |
| SHC1_14 | AGGAAGGGCAGCUGAUGAU | AUCAUCAGCUGCCCUUCCU |
| ZNHIT1_1 | CCGAGGUGAUCAUUUUAAA | UUUAAAAUGAUCACCUCGG |
| ZNHIT1_5 | GUGACCACAUCUUUAAAAU | AUUUUAAAGAUGUGGUCAC |
| ZNHIT1_10 | CUGGAAAGAAAAAGAAGAA | UUCUUCUUUUUCUUUCCAG |
| ZNHIT1_11 | ACACUGGAAAGAAAAAGAA | UUCUUUUUCUUUCCAGUGU |
| LGALS3_3 | GGGAAUUUCUGGUGACAUA | UAUGUCACCAGAAAUUCCC |
| LGALS3_5 | GCAGACGGCUUCUCACUUA | UAAGUGAGAAGCCGUCUGC |
| LGALS3_17 | AGCGGAAAAUGGCAGACAA | UUGUCUGCCAUUUUCCGCU |
| LGALS3_18 | GGGUUAAAAAACUCAAUGA | UCAUUGAGUUUUUUAACCC |

### Example 3

### Experimental Models, Methods and Results Relating to Ocular Disease

The compounds of the present application are tested in the following animal model of Choroidal neovascularization (CNV). This hallmark of wet AMD is induced in model animals by laser treatment.

A) Non-Human Primate Model

CNV induction: Choroidal neovascularization (CNV) is induced by perimacular laser treatment of both eyes prior to dose administration. Nine lesions are placed in the macula with a laser [OcuLight GL (532 nm) Laser Photo-coagulator with an IRIS Medical® Portable Slit Lamp Adaptor], and laser spots in the right eye mirror the placement in the left eye. The approximate laser parameters are as follows: spot size: 50-100 μm diameter; laser power: 300-700 milliwatts; exposure time: 0.1 seconds.

Treatment: Immediately following laser treatment, both eyes of all animals are subjected to a single intravitreal injection. Left eye is typically dosed with 350 ug of synthetic stabilized NOX siRNA in the final volume of 50 ul, whereas the contralateral eye receives 50 ul of PBS (vehicle).

Evaluation
1. All the animals are subjected to daily examination of food consumption and body weight measurements.
2. two monkeys are euthanized at day 6 following CNV induction. Their eyes are enucleated and the posterior pole is flattened. Then the fovea region is excised and separated into choroids and neuroretina which are separately (for every animal) frozen in liquid nitrogen to be subsequently used for RNA extraction and real time PCR evaluation of NOX expression.
3. Fluorescein angiograms are performed pre-study, and at the end of weeks 1, 2, and 3 following CNV induction. Photographs are taken, using a fundus camera (TRC-50EX Retina Camera). Images are captured using the TOPCON IMAGEnet™ system. Fluorescein dye (10% fluorescein sodium, approximately 0.1 mL/kg) is injected via vascular access ports. Photographs are taken at several time points following dye injection, to include the arterial phase, early arteriovenous phase and several late arteriovenous phases in order to evaluate neovascularization and to monitor leakage of fluorescein associated with CNV lesions. Interpretation and analysis of the fluorescein angiograms is independently conducted by two ophthalmologists.

Neovascularization (NV) is assessed in early angiograms and every spot is graded according to the following scheme:
0—no signs of NV
0.5—suspicious spot
1—“hot” spot
2—NV in the laser burn
3—evident NV

Leakage is assessed according to the following scheme:
0—no leakage
0.5—suspicious spot
1—evident small spot leakage
2—leakage growing with time
3—leakage greater than previous borders (evidently)

In addition, the size of every spot is compared between the early and the late angiograms using morphometric measurements, and the increase in the spot's size resulting from the leakage is calculated.

Electroretinograms (ERGs) are recorded using an Epic 2000 electroretinograph according to Sierra's SOPs and the study-specific SOP, including the use of the Ganzfield apparatus, at prestudy and in the end of week 3. A veterinary ophthalmologist evaluates the tabulated ERG data.

C) Efficacy of Combination Therapy of the Target Gene Sirnas

The efficacy of combination therapy of NOX siRNAs and anti-VEGF antibody or aptamer (such as macugen) or additional NOX siRNA sequences in the treatment of diseases in which CNV occurs is tested in the above mouse CNV model.

A) CNV volume studies: The volume of choroidal neovascularization (CNV) 3 weeks after laser injury is computed by confocal fluorescence microscopy as previously described (Sakurai et al. 2003;44: 3578-85 & Sakurai et al. IOVS 2003; 44: 2743-2749).

B) CNV leakage studies

Experiment 1

Materials: Chemically modified NOX siRNA; negative control siRNA (GFP or scrambled); Anti-VEGF antibodies or Macugen® and negative control.

CNV is induced on day zero as described above; the test material is injected to the subjects on day zero and day 7.

The results are evaluated by Fluorescein angiography on weeks 1, 2, 3, and by CNV volume measurement on week 3.

Experimental Groups:

VEGF Ab or Macugen® 0.5 ng/eye, 1 ng/eye, 2 ng/eye, 4 ng/eye

NOX siRNA 0.05 ug/eye, 0.1 ug/eye, 0.25 ug/eye

NOX siRNA:

NOX siRNA 0.05 ug/eye+VEGF Ab or Macugen® 1 ng/eye

NOX siRNA 0.1 ug/eye+VEGF Ab or Macugen® 1 ng/eye

NOX siRNA 0.25 ug/eye+VEGF Ab or Macugen® 1 ng/eye

Control Groups:

PBS

Non-specific IgG 2 ng/eye

negative control 0.1 ug/eye

negative control 0.1 ug/eye+VEGF Ab or Macugen® 1 ng/eye

Experiment 2

This experiment was designed in order to study the effect of test siRNA on gene expression in RPE and neural retina.

Experimental Design

Groups:

PBS

Test siRNA 0.25 mg

CNV is induced by laser treatment as described above on day zero; the test material is also injected on day zero, and the effect evaluated by qPCR analysis of gene expression in RPE and neural retina on days zero and 5.

Additional AMD models which are used to test the methods of the present application: Cc1-2 or Ccr-2 deficient animals—deficiency in either of these proteins causes the development of some of the main features of AMD. Animals deficient in these proteins can be used to test the methods of the present application.

For further information on AMD animal models, see: Chader, Vision research 42 (2002) 393-399; Ambati et al., Nature Medicine 9(11) (2003) 1390-1397; Tolentino et al., Retina 24 (2004) 132-138.

### Example 4

### Models and Results Relating to COPD and Emphysema

The compounds of the present application are tested in the following an animal models and are shown to prevent emphysema:
Cigarette smoke-induced emphysema model: chronic exposure to cigarette smoke causes emphysema in several animal models including mouse, guinea pig.
Lung protease activity as a trigger of emphysema.
VEGFR inhibition model of emphysema.
Bronchial instillation with human neutrophil/pancreatic elastase in rodents.
MMP (matrix metalloprotease)-induced enphysema.
Inflammation-inducedemphysema.

Additionally, emphysema models are generated through genetic means (e.g., mice carrying the TSK mutation), and emphysematous animals may be generated by known modifiers of susceptibility to emphysema such as, inter alia, lung injury, alveolar hypoplasia, hyperoxia, glucocorticoid treatment and nutrition.

Evaluation of the influence of lack of one of the target genes on disease progression in mouse models of emphysema by inhibiting endogenous target gene employing intralung delivery—of siRNA.

CS-induced inflammation is induced by 7 day smoking in 2 groups of C57BL6 mice, 10 mice per group. Group 1: CS+delivery of control siRNA; Group 2: CS+test siRNA. Control groups of mice are instilled with either type of siRNA but kept in room air conditions. The lungs are subsequently agarose-inflated, fixed and imbedded in paraffin, and development oxidative stress in the KO mice is assessed by:

immunohistochemical localization and quantitation of 8-oxo-dG in the lung sections;

immunohistochemical localization and quantitation of active caspase 3 in the lung sections using specific antibodies, or quantitative evaluation of the number of TUNEL-positive cells;

measurement of ceramide concentration in the lung extracts;

measurement of caspase activity in the lung extracts.

Methods

Exposure to Cigarette Smoking (CS)

Exposure is carried out (7 h/day, 7 days/week) by burning 2R4F reference cigarettes (2.45 mg nicotine per cigarette; purchased from the Tobacco Research Institute, University of Kentucky, Lexington, Ky., USA) using a smoking machine (Model TE-10, Teague Enterprises, Davis, Calif., USA). Each smoldering cigarette is puffed for 2 sec, once every minute for a total of eight puffs, at a flow rate of 1.05 L/min, to provide a standard puff of 35 cm3. The smoke machine is adjusted to produce a mixture of sidestream smoke (89%) and mainstream smoke (11%) by burning five cigarettes at one time. Chamber atmosphere is monitored for total suspended particulates and carbon monoxide, with concentrations of 90 mg/m3 and 350 ppm, respectively.

Morphologic and Morphometric Analyses

After exposing the mice to CS or instillation of chemically modified NOX siRNA the mice are anesthetized with halothane and the lungs are inflated with 0.5% low-melting agarose at a constant pressure of 25 cm as previously described. The inflated lungs are fixed in 10% buffered formalin and embedded in paraffin. Sections (5 μm) are stained with hematoxylin and eosin. Mean alveolar diameter, alveolar length, and mean linear intercepts are determined by computer-assisted morphometry with the Image Pro Plus software (Media Cybernetics, Silver Spring, Md., USA). The lung sections in each group are coded and representative images (15 per lung section) are acquired by an investigator masked to the identity of the slides, with a Nikon E800 microscope, 20× lens. The results show that chemically modified test siRNA prevents emphysema caused by smoking as measured by the four parameters described above.

Bronchoalveolar Lavage (BAL) and Phenotyping

Following exposure to CS or instillation of chemically modified NOX siRNA, the mice are anesthetized with sodium pentobarbital. The BAL fluid collected from the lungs of the mice is centrifuged (500 'g at 4° C.), and the cell pellet is resuspended in phosphate-buffered saline. The total number of cells in the lavage fluid is determined, and 2×104 cells are cytocentrifuged (Shandon Southern Products, Pittsburgh, Pa., USA) onto glass slides and stained with Wright-Giemsa stain. Differential cell counts are performed on 300 cells, according to standard cytological techniques .

Identification of Alveolar Apoptotic Cell Populations in the Lungs.

To identify the different alveolar cell types undergoing apoptosis in the lungs, an immunohistochemical staining of active caspase 3 is performed in the lung sections from the room air (RA) as well as CS exposed mice. To identify the apoptotic type II epithelial cells in the lungs, after active caspase 3 labeling, the lung sections are incubated first with anti-mouse surfactant protein C (SpC) antibody and then with an anti-rabbit Texas red antibody. Apoptotic endothelial cells are identified by incubating the sections first with the anti-mouse CD 31 antibody and then with the biotinylated rabbit anti-mouse secondary antibody. The lung sections are rinsed in PBS and then incubated with the streptavidin-Texas red conjugated complex. The apoptotic macrophages in the lungs are identified by incubating the sections first with the rat anti-mouse Mac-3 antibody and then with the anti-rat Texas red antibody. Finally, DAPI is applied to all lung sections, incubated for 5 minutes, washed and mounted with Vectashield HardSet mounting medium. DAPI and fluorescein are visualized at 330-380 nm and 465-495 nm, respectively. Images of the lung sections are acquired with the Nikon E800 microscope, 40× lens.

Immunohistochemical Localization of Active Caspase-3

Immunohistochemical staining of active caspase-3 assay is performed using anti-active caspase-3 antibody and the active caspase-3-positive cells are counted with a macro, using Image Pro Plus program. The counts are normalized by the sum of the alveolar profiles herein named as alveolar length and expressed in gm. Alveolar length correlates inversely with mean linear intercept, i.e., as the alveolar septa are destroyed, mean linear intercepts increases as total alveolar length, i.e., total alveolar septal length decreases.

Caspase 3 Activity Assay

The caspase-3/7 activity is measured in lung tissue extracts using a fluorometric assay according to the manufacturer's instructions. Snap-frozen lung tissue (n=3 per group) was homogenized with the assay buffer, followed by sonication and centrifugation at 800×g. After removal of nuclei and cellular debris, the supernatant (300 μg protein) is then incubated with the pro-fluorescent substrate at room temperature for 1 h and the fluorescence intensity was measured utilizing a Typhoon phosphoimager (Amersham Biosciences, Inc., Piscataway, N.J., USA). The results are expressed as the rate of specific caspase-3 substrate cleavage, expressed in units of caspase 3 enzymatic activity, normalized by total protein concentration. Active recombinant caspase 3 was utilized as the assay standard (0-4 U). Tissue lysates without substrate, assay buffer alone, and lysates with caspase 3 inhibitor were utilized as negative controls.

Immunohistochemical Localization of 8-oxo-dG

For the immunohistochemical localization and quantification of 8-oxo-dG, lung sections from the mice exposed to CS or instilled with chemically modified NOX siRNA are incubated with anti-8-oxo-dG antibody and stained using InnoGenex™ Iso-IHC DAB kit using mouse antibodies. The 8-oxo-dG-positive cells are counted with a macro (using Image Pro Plus), and the counts were normalized by alveolar length as described.

Instillation of siRNA into Mouse Lungs

Chemically modified siRNA (50 ug) is delivered in 80 ul sterile perfluorocarbon. The oxygen carrying properties of perfluorocarbon make it well-tolerated at these volumes, while its physical-chemical properties allow for extremely efficient distal lung delivery when instilled intratracheally. Mice are anesthetized by brief inhalational halothane exposure, the tongue is gently pulled forward by forceps and the trachea instilled with perfluorocarbon solution applied at the base of the tongue via a blunt angiocatheter.

Mice are anesthetized with an intra-peritoneal injection of Ketamine/Xylazine (115/22 mg/kg). 50 μg of siRNA is instilled intranasally in 50 μl volume of 0.9% NaCl by delivering five consecutive 10 μl portions. At the end of the intranasal instillation, the mouse's head is held straight up for 1 minute to ensure that all the instilled solution drains inside.

For further information, see: Rangasamy T, et al., 2004. J.C.I. 114(9):1248-59; Kasahara, Y et al., Am J Respir Crit Care Med Vol 163. pp 737-744, 2001; Kasahara, Y et al., 2000. J. Clin. Invest. 106:1311-1319; and Tuder, R M et al., Pulmonary Pharmacology & Therpaeutics 2002.

### Example 5

### Models and Results Relating to Microvascular Disorders

The compounds of the present application are tested in animal models of a range of microvascular disorders as described below.

1. Retinopathy of Prematurity

Retinopathy of prematurity is induced by exposing the test animals to hypoxic and hyperoxic conditions, and subsequently testing the effects on the retina.

2. Myocardial Infarction

Myocardial infarction is induced by Left Anterior Descending artery ligation in mice, both short term and long term.

3. Microvascular Ischemic Conditions

Animal models for assessing ischemic conditions include:
1. Closed Head Injury (CHI)—Experimental TBI produces a series of events contributing to neurological and neurometabolic cascades, which are related to the degree and extent of behavioral deficits. CHI is induced under anesthesia, while a weight is allowed to free-fall from a prefixed height (Chen et al, J. Neurotrauma 13, 557, 1996) over the exposed skull covering the left hemisphere in the midcoronal plane.
2. Transient middle cerebral artery occlusion (MCAO)—a 90 to 120 minutes transient focal ischemia is performed in adult, male Sprague Dawley rats, 300-370 gr. The method employed is the intraluminal suture MCAO (Longa et al., Stroke, 30, 84, 1989, and Dogan et al., J. Neurochem. 72, 765, 1999). Briefly, under halothane anesthesia, a 3-0-nylon suture material coated with Poly-L-Lysine is inserted into the right internal carotid artery (ICA) through a hole in the external carotid artery. The nylon thread is pushed into the ICA to the right MCA origin (20-23 mm). 90-120 minutes later the thread is pulled off, the animal is closed and allowed to recover.
3. Permanent middle cerebral artery occlusion (MCAO)—occlusion is permanent, unilateral-induced by electrocoagulation of MCA. Both methods lead to focal brain ischemia of the ipsilateral side of the brain cortex leaving the contralateral side intact (control). The left MCA is exposed via a temporal craniectomy, as described for rats by Tamura A., et al., J Cereb Blood Flow Metab. 1981; 1:53-60. The MCA and its lenticulostriatal branch are occluded proximally to the medial border of the olfactory tract with microbipolar coagulation. The wound is sutured, and animals returned to their home cage in a room warmed at 26° C. to 28° C. The temperature of the animals is maintained all the time with an automatic thermostat.

4. Acute Renal Failure (ARF)

Testing active NOX siRNA for treating ARF is done using sepsis-induced ARF or ischemia-reperfusion-induced ARF.

1. Sepsis Induced ARF

Two predictive animal models of sepsis-induced ARF are described by Miyaji T, et al., Kidney Int. 64(5):1620-31. These two models are lipopolysaccharide administration and cecal ligation puncture in mice, preferably in aged mice.

2. Ischemia-Reperfusion-Induced ARF

This predictive animal model is described by Kelly K J, et al., 2003. J Am Soc Nephrol.; 14(1):128-38.

Ischemia-reperfusion injury is induced in rats following 45 minutes bilateral kidney arterial clamp and subsequent release of the clamp to allow 24 hours of reperfusion. Chemically modified siRNA or GFP siRNA (negative control) is injected into the jugular vein 2 hrs prior to and 30 minutes following the clamp. Additional siRNA is given via the tail vein at 4 and 8 hrs after the clamp. ARF progression is monitored by measurement of serum creatinine levels before and 24 hrs post surgery. At the end of the experiment, the rats are perfused via an indwelling femoral line with warm PBS followed by 4% paraformaldehyde. The left kidneys are removed and stored in 4% paraformaldehyde for subsequent histological analysis. Acute renal failure is frequently defined as an acute increase of the serum creatinine level from baseline. An increase of at least 0.5 mg per dL or 44.2 μmol per L of serum creatinine is considered as an indication for acute renal failure. Serum creatinine is measured at time zero before the surgery and at 24 hours post ARF surgery. siRNA to target gene prevents production of ARF in this model.

To study the distribution of siRNA in the rat kidney, Cy3-labeled 19-mer blunt-ended siRNA molecules (2 mg/kg) having alternating O-methyl modification in the sugar residues were administered iv for 3-5 min, after which in vivo imaging was conducted using two-photon confocal microscopy. The confocal microscopy analysis revealed that the majority of siRNA in the kidneys is concentrated in the endosomal compartment of proximal tubular cells. Both endosomal and cytoplasmic siRNA fluorescence were relatively stable during the first 2 hrs post delivery and disappeared at 24 hrs.

The expression of a target gene during ischemia-reperfusion induced ARF is examined in rat kidneys. In both kidney regions, cortex and medulla, NOX transcript level is decreased in the ARF-10 hr group relative to the control group transcript level. Target gene transcript level is also elevated (up-regulated) in the kidney medulla, 3 and 6 hrs following the ARF operation (bilateral renal artery clamp).

### Example 6

### Pharmacology and Drug Delivery

The compounds or pharmaceutical compositions of the present application are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The treatment generally has a length proportional to the length of the disease process and drug effectiveness and the patient species being treated. It is noted that humans are treated generally longer than the mice or other experimental animals exemplified herein.

The compounds of the present application are administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms are prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present application are formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the application.

When administering the compound of the present application parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present application, however, any vehicle, diluent, or additive used have to be compatible with the compounds.

Sterile injectable solutions can be prepared by incorporating the compounds utilized in practicing the present application in the required amount of the appropriate solvent with various of the other ingredients, as desired.

A pharmacological formulation of the present application can be administered to the patient in an injectable formulation containing any compatible carrier, such as various vehicle, adjuvants, additives, and diluents; or the compounds utilized in the present application can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present application include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present application can be administered orally to the patient. Conventional methods such as administering the compound in tablets, suspensions, solutions, emulsions, capsules, powders, syrups and the like are usable. Known techniques which deliver it orally or intravenously and retain the biological activity are preferred. In one embodiment, the compound of the present application can be administered initially by intravenous injection to bring blood levels to a suitable level. The patient's levels are then maintained by an oral dosage form, although other forms of administration, dependent upon the patient's condition and as indicated above, can be used.

In general, the active dose of compound for humans is in the range of from 1 ng/kg to about 20-100 mg/kg body weight per day, preferably about 0.01 mg to about 2-10 mg/kg body weight per day, in a regimen of a single dose, one dose per day or twice or three or more times per day for a period of one day, of several days or of 1-2 weeks or longer, preferably for 24 to 48 hrs or by continuous infusion during a period of 1-2 weeks or longer.

Administration of Compounds of the Present Application to the Eye

The compounds of the present application can be administered to the eye topically or in the form of an injection, such as an intravitreal injection, a sub-retinal injection or a bilateral injection. Preferred methods of delivery to the eye is using siRNA formulated as eye drops.

Further information on administration of the compounds of the present application can be found in Tolentino et al., Retina 24 (2004) 132-138; Reich et al., Molecular vision 9 (2003) 210-216.

Pulmonary Administration of Compounds of the Present Application

The therapeutic compositions of the present application are preferably administered into the lung by inhalation of an aerosol containing these compositions/compounds, or by intranasal or intratracheal instillation of said compositions. Formulating the compositions in liposomes may benefit absorption. Additionally, the compositions may include a PFC liquid such as perflubron, and the compositions formulated as a complex of the compounds of the application with polyethylemeimine (PEI).

For further information on pulmonary delivery of pharmaceutical compositions see Weiss et al., Human gene therapy 10:2287-2293 (1999); Densmore et al., Molecular therapy 1:180-188 (1999); Gautam et al., Molecular therapy 3:551-556 (2001); and Shahiwala & Misra, AAPS Pharm Sci Tech 5 (2004). Additionally, respiratory formulations for siRNA are described in U.S. patent application No. 2004/0063654 of Davis et el.

Further, the compounds of the present application are administered topically where appropriate (such as in the case of diabetic foot ulcers for example), optionally in a lipid/liposome formulation, or for use in iontophoresis.

A preferred administration mode is topical delivery of the siRNA compounds onto the round window membrane of the cochlea as disclosed for example in Tanaka et al. (Hear Res. 2003; 177(1-2):21-31). Preferred delivery to the inner ear comprising administering the siRNA as an eardrop formulation.

In the treatment of pressure sores or other wounds, the administration of the pharmaceutical composition is preferably by topical application to the damages area, but the compositions may also be administered systemically.

Additional formulations for improved delivery of the compounds of the present application can include non-formulated compounds, compounds covalently bound to cholesterol, and compounds bound to targeting antibodies (Song et al., 2005. Nat Biotechnol. 23(6):709-17).

### Example 7

### Model Systems for Pressure Sores or Pressure Ulcers

Pressure sores or pressure ulcers including diabetic ulcers, are areas of damaged skin and tissue that develop when sustained pressure (usually from a bed or wheelchair) cuts off circulation to vulnerable parts of the body, especially the skin on the buttocks, hips and heels. The lack of adequate blood flow leads to ischemic necrosis and ulceration of the affected tissue. Pressure sores occur most often in patients with diminished or absent sensation or who are debilitated, emaciated, paralyzed, or long bedridden. Tissues over the sacrum, ischia, greater trochanters, external malleoli, and heels are especially susceptible; other sites may be involved depending on the patient's situation.

Testing the active siRNA compounds of the application for treating pressure sore, ulcers and similar wounds is done in the mouse model described in Reid R R, et al., J Surgical Research. 116: 172-180, 2004.

Additionally, a rabbit model is described by Mustoe et al, JCI, 1991; Ahn & Mustoe, Ann P1 Surg, 1991 and is used for testing the siRNAs of the application.

### Example 8

### Model Systems for Spinal Cord Injury

Spinal cord injury, or myelopathy, is a disturbance of the spinal cord that results in loss of sensation and/or mobility. The two common types of spinal cord injury are due to trauma and disease. Traumatic injury can be due to automobile accidents, falls, gunshot, diving accidents inter alia, and diseases which can affect the spinal cord include polio, spina bifida, tumors and Friedreich's ataxia.

Testing the active siRNA compounds of the application for treating spinal cord injury is done in the rat spinal cord contusion model as described by Young, W. in Prog Brain Res. 2002;137:231-55. Other predictive animal models of spinal cord injury are described in the following references: Gruner, J A 1992. J Neurotrauma 9(2): 123; Hasegawa, K. and M. Grumet 2003. J Neurosurg 98(5): 1065-71; and Huang, P P and W. Young (1994). J Neurotrauma 11(5): 547.

### Example A

### Efficacy Using Intranasal Administration of siRNA Compounds in a Rat Model of Spinal Cord Injury

Objective. The objective of this study is to test functional recovery after intranasal administration of an siRNA compound of the application following mild SCI.

Animals and Treatment. The study includes eighteen (18) female SD rats of 77+1 day age that are randomly divided into three equal groups. The animals are subjected to mild spinal cord injury, as described in Basso D M, et al. (Exp Neurol. 1996 June; 139(2):244-56) by using 12.5 mm weight drop and are subsequently treated as described in Table Example 8A.

TABLE EXAMPLE 8A

| | | | | | | Total |
|---|---|---|---|---|---|---|
| | | | | | | amount |
| | | siRNA | | | Termi- | of |
| | | delivery | siRNA | Instillation | nation | siRNA/ |
| Group | siRNA | method | dose | regime | time | rat |
| A | siRNA | Intra- | 300 - 600 | 1st week - | 6 weeks | 5.4 mg |
| | NOX2 | nasal | μg/total | 3 times | | |
| B | siRNA | | | a week | | |
| | RhoA | | | 2nd week - | | |
| C | siRNA | | | 3 times | | |
| | CLN_1 | | | a week | | |
| | (Control) | | | 3d week - | | |
| | | | | 3 times | | |
| | | | | a week | | |

Tissue harvesting for analysis. Three (3) rats in each group are perfused with cold PBS, and spinal cord and brain are dissected as follows. Spinal cord is divided into 5 pieces (centered at the impact site) as well as a piece from T1 as a control (uninjured region) for each cord, and separately fresh frozen and placed in Trizol. Brain is dissected into the following regions: 1. Left/right Olfactory bulbs; 2. Cerebellum; 3. Brain stem; 4. Left/right cerebral cortex; 5. Pons; 6. Hippocampus Left/Right. Each region is separately fresh frozen and stored in Trizol.

One (1) rat in each group is perfused with 4% PFA, and whole spinal cord and longitudinally dissected brain is stored in 4% PFA.

In the remaining two (2) rats in each group, spinal cord and brain are dissected as follows: Spinal cord is divided into 5 pieces (centered at the impact site) as well as a piece from T1 as a control (uninjured region) for each cord. Brain is dissected into: 1. cerebral cortex (L/R); 2. hippocampus; 3. cerebellum.

Evaluation. The following test are performed:

1. Assessment of behavioral outcome. The effect of treatment on restoring the locomotor function is analyzed by applying the Basso, Beattie and Bresnahan (BBB) locomotor score (Baaso et al., 1995).

2. Molecular Biology. RhoA activity is tested using rhotectin (pull down assay). siRNA NOX2 , siRNA RhoA_{—}4 and CLN_{—}1 are detected by qPCR and ISH.

### Example 9

### Model Systems for Glaucoma

Testing of the active siRNA compounds of the application for treating or preventing Glaucoma is done in the animal model for example as described by Pease et al., J. Glaucoma, 2006, 15(6):512-9 (Manometric calibration and comparison of TonoLab and TonoPen tonometers in rats with experimental glaucoma and in normal mice).

Rat Optic Nerve Crush (ONC) Model: Intravitreal siRNA Delivery and Eye Drop Delivery

For optic nerve transsection the orbital optic nerve (ON) of anesthetized rats is exposed through a supraorbital approach, the meninges severed and all axons in the ON transected by crushing with forceps for 10 seconds, 2 mm from the lamina cribrosa.

The siRNA compounds are delivered alone or in combination in 5 uL volume (10 ug/uL) as eye drops. Immediately after optic nerve crush (ONC), 20 ug/10 ul test siRNA or 10 ul PBS is administered to one or both eyes of adult Wistar rats and the levels of siRNA taken up into the dissected and snap frozen whole retinae at 5 h and 1 d, and later at 2 d, 4 d, 7 d, 14 d and 21 d post injection is determined. Similar experiments are performed in order to test activity and efficacy of siRNA administered via eye drops.

### Example 10

### Animal Models of Allograft Transplant

The animal models disclosed in the following publications are useful for testing the compounds of the present application, in particular siRNA that target the NOX target genes including NOX2 (CYBB) and NOX4, p53, HTRA2, KEAP1, SHC1, ZNHIT1, LGALS3 and HI95, or any combination thereof.

Yasufuku et al., Transplantation. 2002 73(4):500-5. (Erratum in: Transplantation 2002 May 15; 73(9):1529.) Prevention of bronchiolitis obliterans in rat lung allografts by type V collagen-induced oral tolerance.

Krupnick et al., Nat Protoc. 2009; 4(1):86-93. Orthotopic mouse lung transplantation as experimental methodology to study transplant and tumor biology.

Mizobuchi et al., J Heart Lung Transplant. 2004 23(7):889-93. Comparison of surgical procedures for vascular and airway anastomoses that utilize a modified non-suture external cuff technique for experimental lung transplantation in rats.

Wilkes, et al., 1999. Transplantation 67:890-896. Allergenic bronchoalveolar lavage cells induce the histology and immunology of lung allograft rejection in recipient murine lungs: role of intercellular adhesion molecule-1 on donor cells.

siRNA compounds of the present application are tested in these animal models, which show that the siRNA compounds treat and/or prevent complications following lung transplantation and thus may be used in conjunction with transplant surgery.

### Example 11

### Prevention of Primary Graft Dysfunction in Rat Lung Transplantation Model Using siRNA Compounds

Design: Orthotopic left lung transplantation is performed on SD rats, with 1 h cold ischemia time according to the method previously described (Yasufuku et al., 2001. “Oral tolerance induction by type V collagen Downregulates Lung Allograft rejection”, Am J Respir Cell Mol Biol, 25:26-34; Yoshida et al., 2007 “Metalloproteinase inhibition has differential effects on alloimmunity, autoimmunity, and histopathology in the transplanted lungs” Transplantation, 27:683-684).

After the donor rats are anesthetized with an intramuscular injection of ketamine/xylazine, the chest is shaved, a sternotomy is performed and the heart and lungs is removed en bloc. The donor lung is wrapped in sterile gauze saturated with saline and placed on ice (4° C.) in a sterile beaker for 1 h followed by orthotopic transplantation to recipient rats. A siRNA compound of the application is administered intratracheally to the recipients at a dose of 350 mg/500 ml sterile saline/rat, 15-30 minutes after anastomosis/reperfusion.

Bronchoalveolar lavage fluid (BALF) is collected from the transplanted (left) and control right lung 4 h, 24 h and 5 d after the induction of ischemia in the recipients. BALF is collected by 5 subsequent lavages with 1 ml of warm PBS. The lungs are then perfused with saline and blood is collected for serum isolation.

At 24 hours post lung transplantation, pulmonary function is determined by evaluating elastance, compliance and resistance in a single-compartment model, by evaluating total lung capacity and/or by using the constant phase model.

### Example 12

### Model Systems for Acute Lung Injury (ALI)

Intratracheal (i.t) administration of LPS (Lipopolysaccharide), a bacterial cell wall component, is an accepted experimental model of acute lung injury (ALI), as LPS stimulates profound lung recruitment of inflammatory cells and the subsequent development of systemic inflammation.

(See, for example, Fang W F, et al., Am J Physiol Lung Cell Mol Physiol. 2007 293(2):L336-44; Hagiwara S, Iwasaka H, Noguchi T. J Anesth. 2007; 21(2):164-70).

Time-dependent changes of NOX gene expression in mice lungs during the first 24 hours (time points 0.5; 1; 2; 4; 8 & 24 hours), after Intratracheal (i.t) administration of LPS is assessed. The assessment of gene expression is done using qPCR.

### Example 13

### Model Systems for Acute Respiratory Distress Syndrome

Testing of the siRNA compound for treating Acute respiratory distress syndrome is performed, inter alia, in the animal model as described by Chen et al. in J Biomed Sci. 2003; 10(6 Pt 1):588-92.

### Example 14

### Model Systems for Hearing Loss Conditions

(i) Animal Model of Carboplatin-Induced or Cisplatin-Induced Hair Cell Death in the Cochlea of Chinchilla:

Chinchillas are pre-treated by direct administration of target gene specific siRNAs in saline or another formulation to the left ear of each animal. Saline is given to the right ear of each animal as placebo. Two days following the administration of the specific siRNA, the animals are treated with carboplatin (75 mg/kg ip) or cisplatin (intraperitoneal infusion of 13 mg/kg over 30 minutes). After sacrifice of the chinchillas (two weeks post carboplatin treatment) the percentage of dead cells of inner hair cells (IHC) and outer hair cells (OHC) is calculated in the left ear (siRNA treated) and in the right ear (saline treated).The percentage of dead cells is lower in the siRNA treated ear than in the control

(ii) Animal Model of Acoustic-Induced Hair Cell Death in the Cochlea of Chinchilla:

The activity of target specific siRNA in an acoustic trauma model is studied in chinchilla. The animals are exposed to an octave band of noise centered at 4 kHz for 2.5 h at 105 dB. The left ear of the noise-exposed chinchillas is pre-treated (48 h before the acoustic trauma) with 30 μg of either siRNA in ˜10 μL of saline; the right ear is pre-treated with vehicle (saline). The compound action potential (CAP) is a convenient and reliable electrophysiological method for measuring the neural activity transmitted from the cochlea. The CAP is recorded by placing an electrode near the base of the cochlea in order to detect the local field potential that is generated when a sound stimulus, such as click or tone burst, is abruptly turned on. The functional status of each ear is assessed 2.5 weeks after the acoustic trauma. Specifically, the mean threshold of the compound action potential recorded from the round window is determined 2.5 weeks after the acoustic trauma in order to determine if the thresholds in the siRNA-treated ear are lower (better) than the untreated (saline) ear. In addition, the amount of inner and outer hair cell loss is determined in the siRNA-treated and the control ear. It is found that the thresholds in the siRNA-treated ear are lower than the untreated (saline) ear Also, the amount of hair cell loss is lower in the siRNA-treated ear than in the control ear.

### Example 15

### Model Systems of Acute Renal Failure (ARF)

ARF is a clinical syndrome characterized by rapid deterioration of renal function that occurs within days. Without being bound by theory the acute kidney injury may be the result of renal ischemia-reperfusion injury such as renal ischemia-reperfusion injury in patients undergoing major surgery such as major cardiac surgery. The principal feature of ARF is an abrupt decline in glomerular filtration rate (GFR), resulting in the retention of nitrogenous wastes (urea, creatinine) Recent studies support the hypothesis that apoptosis in renal tissues is prominent in most human cases of ARF. The principal site of apoptotic cell death is the distal nephron. During the initial phase of ischemic injury, loss of integrity of the actin cytoskeleton leads to flattening of the epithelium, with loss of the brush border, loss of focal cell contacts, and subsequent disengagement of the cell from the underlying substratum.

Testing of the active siRNA for each target gene separately for treating ARF is done using an animal model for ischemia-reperfusion-induced ARF.

Ischemia-reperfusion induced ARF: Ischemia-reperfusion injury is induced in rats following 45 minutes bilateral kidney arterial clamp and subsequent release of the clamp to allow 24 hours of reperfusion. Twelve mg/kg of siRNA of the application (i.e. siRNA to a specific pro-apoptotic gene) are injected into the jugular vein 30 minutes prior to and 4 hours following the clamp. ARF progression is monitored by measurement of serum creatinine levels before (baseline) and 24 hrs post surgery. At the end of the experiment, the rats are perfused via an indwelling femoral line with warm PBS followed by 4% paraformaldehyde. The left kidneys are removed and stored in 4% paraformaldehyde for subsequent histological analysis. Acute renal failure is frequently defined as an acute increase of the serum creatinine level from baseline. An increase of at least 0.5 mg per dL or 44.2 μmol per L of serum creatinine is considered as an indication for acute renal failure. Serum creatinine is measured at time zero before the surgery and at 24 hours post ARF surgery. The results show that the siRNA compounds of the application prevent onset of acute renal failure in this model.

### Example 16

### Model Systems for Transplantation-Associated Acute Kidney Injury

Warm ischemia—A left nephrectomy was performed, followed by auto transplantation that resulted in a warm kidney graft preservation period of 45 minutes. Following auto transplantation, a right nephrectomy was performed on the same animal. A siRNA compound targeting P53 is administered intravenously via the femoral vein either before harvesting of the kidney graft (mimicking donor treatment) (“pre”), or after the kidney autotransplantation (mimicking recipient treatment), or both before harvest and after transplantation (combined donor and recipient treatment) (“pre-post”).

Cold ischemia—A left nephrectomy is performed on a donor animal, followed by a cold preservation (on ice) of the harvested kidney for a period of 5 hours. At the end of this period, the recipient rat undergoes a bilateral nephrectomy, followed by transplantation of the cold-preserved kidney graft. The total warm ischemia time (including surgical procedure) is 30 minutes. SiRNA is administered intravenously via the femoral vein, either to the donor animal prior to the kidney harvest (“pre”), or to the recipient animal 15 minutes (“post 15 min”) or 4 hours (post 4 hrs) post-transplantation.

To assess the efficacy of test siRNA in improvement of post-transplantation renal function, serum creatinine levels are measured on days 1, 2, and 7 post-transplantation in both warm and cold ischemia models.

### Example 17

### Model Systems for Neurodegenerative Diseases and Disorders

### Example A

### Evaluating the Efficacy of Intranasal Administration of siRNA Compounds in a Mouse Model of Alzheimer's Disease

Animals and Treatment. The study includes twenty-four (24) APP [V717I] transgenic mice (female), a model for Alzheimer's disease (Moechars D. et al., EMBO J. 15(6):1265-74, 1996; Moechars D. et al., Neuroscience. 91(3):819-30), aged 11 months that are randomly divided into two equal groups (Group I and Group II).

Animals are treated with intranasal administration of: NOX2 siRNA or SHC 1 siRNA or a combination thereof, (200-400 μg/mice, Group I) and vehicle (Group II), 2-3 times a week, during 3 months.

Termination. Mice are sacrificed; brains are dissected and process one hemisphere for histology and freeze one hemisphere for shipment.

Evaluation. The following histological analysis is performed:

1. Anti-Aβ staining and quantification (4 slides/mouse):

2. Thio S staining and quantification (4 slides/mouse):

3. CD45 staining and quantification (4 slides/mouse):

4. GFAP (astrocytosis) staining and quantification:

### Example B

### Evaluating the Efficacy of Intranasal Administration of siRNA in a Mouse Model of ALS

Objective. To examine the efficacy of siRNA to target genes (i.e. NOX2 and SHC1) in the mutant SOD1^{G93A} mouse model of ALS.

Animals and Treatment. The following experimental groups are used for studying disease progression and lifespan:

1. Group 1—Mismatch siRNA—wild-type (n=10) and SOD1^{G93A} mice (n=10)

2. Group 2—NOX siRNA—wild-type (n=10) and SOD1^{G93A} mice (n=10)

3. Group 3—Untreated controls—wild-type (n=10) and SOD1^{G93A} mice (n=10)

Each experimental group is sex matched (5 male, 5 female) and contain littermates from at least 3 different litters. This design reduces bias that may be introduced by using mice from only a small number of litters, or groups of mice with a larger percentage of female SOD1^{G93A} mice (since these mice live 3-4 days longer than males).

Administration of siRNA. The route of administration of the siRNA is intranasal, with administration twice weekly, starting from 30 days of age.

Analysis of disease progression. Behavioral and electromyography (EMG) analysis in treated and untreated mice is performed to monitor disease onset and progression. Mice are pre-tested before start of siRNA treatment, followed by weekly assessments. All results are compared statistically. The following tests are performed:

1. Swimming tank test: this test is particularly sensitive at detecting changes in hind-limb motor function (Raoul et al., 2005. Nat Med. 11, 423-428; Towne et al, 2008. Mol Ther. 16: 1018-1025).

2. Electromyography: EMG assessments are performed in the gastrocnemius muscle of the hind limbs, where compound muscle action potential (CMAP) is recorded (Raoul et al., 2005. supra).

3. Body weight: The body weight of mice is recorded weekly, as there is a significant reduction in the body weight of SOD1^{G93A} mice during disease progression (Kieran et al., 2007. PNAS USA. 104, 20606-20611).

Assessment of lifespan. The lifespan in days for treated and untreated mice is recorded and compared statistically to determine whether siRNA treatment has any significant effect on lifespan. Mice are sacrificed at a well-defined disease end point, when they have lost >20% of body weight and are unable to raise themselves in under 20 seconds. All results are compared statistically.

Post mortem histopathology. At the disease end-point mice are terminally anaesthetised and spinal cord and hind-limb muscle tissue are collected for histological and biochemical analysis.

Examining motoneuron survival. Transverse sections of lumbar spinal cord are cut using a cryostat and stained with gallocyanin, a nissl stain. From these sections the number of motoneurons in the lumbar spinal cord is counted (Kieran et al., 2007. supra), to determine whether siRNA treatment prevents motoneuron degeneration in SOD1 ^{G93A} mice.

Examining spinal cord histopathology. Motoneuron degeneration in SOD1^{G93A} mice results in astrogliosis and activation of microglial cells. Here, using transverse sections of lumbar spinal cord the activation of astocytes and microglial cells is examined using immunocytochemistry to determine whether siRNA treatment reduced or prevented their activation.

Examining muscle histology. Hind-limb muscle denervation and atrophy occur as a consequence of motoneuron degeneration in SOD1^{G93A} mice. At the disease end point the weight of individual hind-limb muscles (gastrocnemius, soleus, tibialis anterior, extensor digitorium longus muscles) is recorded and compared between treated and untreated mice. Muscles are then processed histologicaly to examine motor end plate denervation and muscle atrophy (Kieran et al., 2005. J Cell Biol. 169, 561-567).

Tables A1-A18 and B1-B15 which set forth sense and antisense oligonucleotide pairs uuseful in preparing siRNA compounds according to the present application are presented herein below.

TABLE A1
NOX4 19-mer oligomer pairs (NADPH oxidase 4)

| | | | | Human- |
|---|---|---|---|---|
| | | | | 20149638 |
| No. | Sense siRNA | AntiSense siRNA | Other Sp | ORF: 240-1976 |
| 1 | GCCCUUCAUUCAAUCUAGA | UCUAGAUUGAAUGAAGGGC | Chmp, Rat | [1427-1445] |
| 2 | CAUCCAUUUACCCUCACAA | UUGUGAGGGUAAAUGGAUG | Chmp | [1293-1311] |
| 3 | UCAUCCAUUUACCCUCACA | UGUGAGGGUAAAUGGAUGA | Chmp | [1292-1310] |
| 4 | GCCUUAUCCUUUUACCCAU | AUGGGUAAAAGGAUAAGGC | | [445-463] |
| 5 | CAAUACUCAACACCCUGUU | AACAGGGUGUUGAGUAUUG | | [1558-1576] |
| 6 | CCCUUCAUUCAAUCUAGAA | UUCUAGAUUGAAUGAAGGG | Chmp, Rat | [1428-1446] |
| 7 | GGACUCUAAAGAAGGAAUG | CAUUCCUUCUUUAGAGUCC | Chmp | [1996-2014] |
| 8 | GGAAACUUUUGUUUGAUGA | UCAUCAAACAAAAGUUUCC | Chmp | [1816-1834] |
| 9 | CCGUUGGUUUGCAGAUUUA | UAAAUCUGCAAACCAACGG | Chmp | [1646-1664] |
| 10 | CUGCCCUUCAUUCAAUCUA | UAGAUUGAAUGAAGGGCAG | Chmp, Rat | [1425-1443] |
| 11 | GAGUAUUUCUCAGAACAUU | AAUGUUCUGAGAAAUACUC | Chmp | [960-978] |
| 12 | CCAUGAAUGUCCUGCUUUU | AAAAGCAGGACAUUCAUGG | Chmp | [310-328] |
| 13 | CUGCCUCCAUCUAGUCAAG | CUUGACUAGAUGGAGGCAG | Chmp | [1395-1413] |
| 14 | GGGUUCAUCUUAAAAUAGU | ACUAUUUUAAGAUGAACCC | Chmp | [1342-1360] |
| 15 | CCAACUGAAACCAAAGCAA | UUGCUUUGGUUUCAGUUGG | Chmp | [1317-1335] |
| 16 | CGAAAGACUUUACAGGUAU | AUACCUGUAAAGUCUUUCG | Chmp | [1127-1145] |
| 17 | CCGAAAGACUUUACAGGUA | UACCUGUAAAGUCUUUCGG | Chmp | [1126-1144] |
| 18 | GGAUUUUCAAAACCGGCAG | CUGCCGGUUUUGAAAAUCC | Chmp | [999-1017] |
| 19 | GGAGAUUGUUGGAUAAAAG | CUUUUAUCCAACAAUCUCC | Chmp | [523-541] |
| 20 | GCACACUGUUGAUUUUCAU | AUGAAAAUCAACAGUGUGC | | [2237-2255] |
| 21 | GGAGAAAAAUAUCAUGCAC | GUGCAUGAUAUUUUUCUCC | Chmp | [1764-1782] |
| 22 | UGCCCUUCAUUCAAUCUAG | CUAGAUUGAAUGAAGGGCA | Chmp, Rat | [1426-1444] |
| 23 | UGCUAUUCCUCAUGAUCAC | GUGAUCAUGAGGAAUAGCA | Chmp | [748-766] |
| 24 | GUGGAAACUUUUGUUUGAU | AUCAAACAAAAGUUUCCAC | Chmp | [1814-1832] |
| 25 | UCAAUACUCAACACCCUGU | ACAGGGUGUUGAGUAUUGA | | [1557-1575] |
| 26 | GUAUAUUGAUGGUCCUUUU | AAAAGGACCAUCAAUAUAC | Chmp | [1460-1478] |
| 27 | ACUGCCUCCAUCUAGUCAA | UUGACUAGAUGGAGGCAGU | Chmp | [1394-1412] |
| 28 | GGAGACUGGACAGAACGAU | AUCGUUCUGUCCAGUCUCC | Chmp, Ms | [1362-1380] |
| 29 | CACAAUGUGUCCAACUGAA | UUCAGUUGGACACAUUGUG | Chmp | [1307-1325] |
| 30 | CCGGCUGCAUCAGUCUUAA | UUAAGACUGAUGCAGCCGG | Chmp | [910-928] |
| 31 | CUAUUCCUCAUGAUCACAG | CUGUGAUCAUGAGGAAUAG | Chmp | [750-768] |
| 32 | UCACAACUGUUCCUGGCCU | AGGCCAGGAACAGUUGUGA | Chmp | [706-724] |
| 33 | UGAGGAUCCUAGAAAACUU | AAGUUUUCUAGGAUCCUCA | Chmp | [683-701] |
| 34 | CAACUUCUCAGUGAAUUAC | GUAAUUCACUGAGAAGUUG | Chmp | [623-641] |
| 35 | AGGAGAUUGUUGGAUAAAA | UUUUAUCCAACAAUCUCCU | Chmp | [522-540] |
| 36 | CCUUAUCCUUUUACCCAUG | CAUGGGUAAAAGGAUAAGG | | [446-464] |
| 37 | AGGACUCUAAAGAAGGAAU | AUUCCUUCUUUAGAGUCCU | Chmp | [1995-2013] |
| 38 | CCCAAUUCACUAUCCAAGA | UCUUGGAUAGUGAAUUGGG | Chmp | [1884-1902] |
| 39 | GACCCAAUUCACUAUCCAA | UUGGAUAGUGAAUUGGGUC | Chmp | [1882-1900] |
| 40 | GCAUAACAAGUUUUGGCAA | UUGCCAAAACUUGUUAUGC | Chmp, Rat, Ms | [1676-1694] |
| 41 | GGCUAGGAUUGUGUCUAAG | CUUAGACACAAUCCUAGCC | Chmp | [391-409] |
| 42 | CAGAACGAUUUCGAGAUUU | AAAUCUCGAAAUCGUUCUG | Chmp | [1372-1390] |
| 43 | CGAAUGGUCAAAGAAAAUU | AAUUUUCUUUGACCAUUCG | Chmp | [1212-1230] |
| 44 | CUCAUGAUCACAGCCUCUA | UAGAGGCUGUGAUCAUGAG | Chmp | [756-774] |
| 45 | UGGUGUUACUAUCUGUAUU | AAUACAGAUAGUAACACCA | Chmp | [563-581] |
| 46 | GGUUGAACAAAUGCAAGAU | AUCUUGCAUUUGUUCAACC | Chmp | [2338-2356] |
| 47 | CUAGUGAGGAGCUGAACUU | AAGUUCAGCUCCUCACUAG | Chmp | [2274-2292] |
| 48 | GGAAUCAAUCAGCUGUGUU | AACACAGCUGAUUGAUUCC | Chmp | [2045-2063] |
| 49 | CGGAAUCAAUCAGCUGUGU | ACACAGCUGAUUGAUUCCG | Chmp | [2044-2062] |
| 50 | GCAAUUUCUAAGACUUUGA | UCAAAGUCUUAGAAAUUGC | Chmp | [2018-2036] |
| 51 | ACCCAAUUCACUAUCCAAG | CUUGGAUAGUGAAUUGGGU | Chmp | [1883-1901] |
| 52 | AGGAAAAACAGUUGGUGUU | AACACCAACUGUUUUUCCU | | [1853-1871] |
| 53 | UGGAAACUUUUGUUUGAUG | CAUCAAACAAAAGUUUCCA | Chmp | [1815-1833] |
| 54 | CUGAAUUCAAGACUGUUUA | UAAACAGUCUUGAAUUCAG | Chmp | [1782-1800] |
| 55 | CAGAAGAUAAUUGGAGAAA | UUUCUCCAAUUAUCUUCUG | Chmp, Rat, Ms | [1752-1770] |
| 56 | GUAACUCCAUUUGCAUCAA | UUGAUGCAAAUGGAGUUAC | Chmp | [1542-1560] |
| 57 | AGAAAAUCAUCCAUUUACC | GGUAAAUGGAUGAUUUUCU | Chmp | [1286-1304] |
| 58 | GAAAGACUUUACAGGUAUA | UAUACCUGUAAAGUCUUUC | Chmp | [1128-1146] |
| 59 | GAAAACUUCUCUUCACAAC | GUUGUGAAGAGAAGUUUUC | Chmp | [694-712] |
| 60 | GGAUCCUAGAAAACUUCUC | GAGAAGUUUUCUAGGAUCC | Chmp | [686-704] |
| 61 | AGACUUUGUUGAACUGAAU | AUUCAGUUCAACAAAGUCU | | [647-665] |
| 62 | GUUGAACAAAUGCAAGAUU | AAUCUUGCAUUUGUUCAAC | Chmp | [2339-2357] |
| 63 | CUUAUCCUUUUACCCAUGU | ACAUGGGUAAAAGGAUAAG | | [447-465] |
| 64 | CUGCAGCCUUAUCCUUUUA | UAAAAGGAUAAGGCUGCAG | Chmp | [440-458] |
| 65 | UGCCAAAGAAUAGUAAGGU | ACCUUACUAUUCUUUGGCA | Chmp | [2065-2083] |
| 66 | AGAAGGAAUGAGUGCAAUU | AAUUGCACUCAUUCCUUCU | Chmp | [2005-2023] |
| 67 | AAAGAAGGAAUGAGUGCAA | UUGCACUCAUUCCUUCUUU | Chmp | [2003-2021] |
| 68 | CUCUAAAGAAGGAAUGAGU | ACUCAUUCCUUCUUUAGAG | Chmp | [1999-2017] |
| 69 | CAGGACUCUAAAGAAGGAA | UUCCUUCUUUAGAGUCCUG | Chmp | [1994-2012] |
| 70 | CCAAGACUCUUCAUAAACU | AGUUUAUGAAGAGUCUUGG | Chmp | [1897-1915] |
| 71 | CUGUUCUUAACCUCAACUG | CAGUUGAGGUUAAGAACAG | Chmp | [424-442] |
| 72 | CAAUUCACUAUCCAAGACU | AGUCUUGGAUAGUGAAUUG | Chmp | [1886-1904] |
| 73 | CAUCUGUUCUUAACCUCAA | UUGAGGUUAAGAACAGAUG | Chmp | [421-439] |
| 74 | GCAAAAUAUAACAGAGGAA | UUCCUCUGUUAUAUUUUGC | | [1839-1857] |
| 75 | GGUGGAAACUUUUGUUUGA | UCAAACAAAAGUUUCCACC | Chmp | [1813-1831] |
| 76 | UCGGUGGAAACUUUUGUUU | AAACAAAAGUUUCCACCGA | Chmp | [1811-1829] |
| 77 | AGAAAAAUAUCAUGCACUG | CAGUGCAUGAUAUUUUUCU | Chmp | [1766-1784] |
| 78 | ACAGAAGAUAAUUGGAGAA | UUCUCCAAUUAUCUUCUGU | Chmp, Rat | [1751-1769] |
| 79 | CUAGGAUUGUGUCUAAGCA | UGCUUAGACACAAUCCUAG | Chmp | [393-411] |
| 80 | CCCAAGCUGUAUAUUGAUG | CAUCAAUAUACAGCUUGGG | Chmp | [1452-1470] |
| 81 | ACGAUUUCGAGAUUUACUA | UAGUAAAUCUCGAAAUCGU | Chmp | [1376-1394] |
| 82 | GACAGAACGAUUUCGAGAU | AUCUCGAAAUCGUUCUGUC | Chmp | [1370-1388] |
| 83 | ACAAUGUGUCCAACUGAAA | UUUCAGUUGGACACAUUGU | Chmp | [1308-1326] |
| 84 | ACCCUCACAAUGUGUCCAA | UUGGACACAUUGUGAGGGU | Chmp | [1302-1320] |
| 85 | CCUGUACUGUGCCGAAAGA | UCUUUCGGCACAGUACAGG | Chmp | [1115-1133] |
| 86 | CAGAUUCCAAGCUAAUUUU | AAAAUUAGCUUGGAAUCUG | Chmp | [1061-1079] |
| 87 | CAAACUAAUUUAGAUACCC | GGGUAUCUAAAUUAGUUUG | Chmp | [885-903] |
| 88 | AGAUGAGGAUCCUAGAAAA | UUUUCUAGGAUCCUCAUCU | Chmp | [680-698] |
| 89 | CGAGAUGAGGAUCCUAGAA | UUCUAGGAUCCUCAUCUCG | Chmp | [678-696] |
| 90 | UGAAUUACAGUGAAGACUU | AAGUCUUCACUGUAAUUCA | | [634-652] |
| 91 | UCGUGUUCCUCUUUAAAUU | AAUUUAAAGAGGAACACGA | Chmp | [2313-2331] |
| 92 | GAGACCCUGAAGAAUAUUU | AAAUAUUCUUCAGGGUCUC | Chmp | [2168-2186] |
| 93 | ACAUGUUUAAUCUGGAAAC | GUUUCCAGAUUAAACAUGU | Chmp | [2145-2163] |
| 94 | GCAGCCUUAUCCUUUUACC | GGUAAAAGGAUAAGGCUGC | Chmp | [442-460] |
| 95 | GUUAUGCCAAAGAAUAGUA | UACUAUUCUUUGGCAUAAC | Chmp | [2061-2079] |
| 96 | UGAGUGCAAUUUCUAAGAC | GUCUUAGAAAUUGCACUCA | Chmp | [2013-2031] |
| 97 | ACAAUAAAGAGUCUUUCAG | CUGAAAGACUCUUUAUUGU | Chmp | [1954-1972] |
| 98 | ACAACUCAUAUGGGACAAG | CUUGUCCCAUAUGAGUUGU | | [1927-1945] |
| 99 | GUGGACCCAAUUCACUAUC | GAUAGUGAAUUGGGUCCAC | Chmp | [1879-1897] |
| 100 | CUCGGUGGAAACUUUUGUU | AACAAAAGUUUCCACCGAG | Chmp | [1810-1828] |
| 101 | GAGAAAAAUAUCAUGCACU | AGUGCAUGAUAUUUUUCUC | Chmp | [1765-1783] |
| 102 | UGGAGAAAAAUAUCAUGCA | UGCAUGAUAUUUUUCUCCA | Chmp | [1763-1781] |
| 103 | ACAAGUUUUGGCAAGAGAA | UUCUCUUGCCAAAACUUGU | Chmp | [1681-1699] |
| 104 | UGAGGAAUCACUGAACUAU | AUAGUUCAGUGAUUCCUCA | Chmp | [1490-1508] |
| 105 | GCUGUAUAUUGAUGGUCCU | AGGACCAUCAAUAUACAGC | Chmp | [1457-1475] |
| 106 | CAAGCUGUAUAUUGAUGGU | ACCAUCAAUAUACAGCUUG | Chmp | [1454-1472] |
| 107 | UGGUCAGUAUAUUACUCUA | UAGAGUAAUAUACUGACCA | Chmp | [1244-1262] |
| 108 | CCGAAUGGUCAAAGAAAAU | AUUUUCUUUGACCAUUCGG | Chmp | [1211-1229] |
| 109 | AGUUUACCCAGCACAAAUU | AAUUUGUGCUGGGUAAACU | Chmp | [1018-1036] |
| 110 | GAGUUUACCCAGCACAAAU | AUUUGUGCUGGGUAAACUC | Chmp | [1017-1035] |
| 111 | UCCUCAUGAUCACAGCCUC | GAGGCUGUGAUCAUGAGGA | Chmp | [754-772] |
| 112 | CUCCAUGAAUGUCCUGCUU | AAGCAGGACAUUCAUGGAG | Chmp | [308-326] |
| 113 | GUGAAUUACAGUGAAGACU | AGUCUUCACUGUAAUUCAC | | [633-651] |
| 114 | CAGUGAAUUACAGUGAAGA | UCUUCACUGUAAUUCACUG | | [631-649] |
| 115 | CCGAGGAUCACAGAAGGUU | AACCUUCUGUGAUCCUCGG | Chmp | [488-506] |
| 116 | CCGAACACUCUUGGCUUAC | GUAAGCCAAGAGUGUUCGG | Chmp | [467-485] |
| 117 | CUUUUACCCAUGUGCCGAA | UUCGGCACAUGGGUAAAAG | | [453-471] |
| 118 | UGAUUUUCAUGGUGGAUUC | GAAUCCACCAUGAAAAUCA | | [2246-2264] |
| 119 | AGGAAUGAGUGCAAUUUCU | AGAAAUUGCACUCAUUCCU | Chmp | [2008-2026] |
| 120 | AAGAAGGAAUGAGUGCAAU | AUUGCACUCAUUCCUUCUU | Chmp | [2004-2022] |
| 121 | UGGGACAAGAUUUGAAUAC | GUAUUCAAAUCUUGUCCCA | | [1937-1955] |
| 122 | GCAUCUGUUCUUAACCUCA | UGAGGUUAAGAACAGAUGC | Chmp | [420-438] |
| 123 | ACUGAAUUCAAGACUGUUU | AAACAGUCUUGAAUUCAGU | Chmp | [1781-1799] |
| 124 | GGGAUACAGAAGAUAAUUG | CAAUUAUCUUCUGUAUCCC | Chmp, Rat | [1746-1764] |
| 125 | CUCUGUAUGUUGCAUAACA | UGUUAUGCAACAUACAGAG | Chmp | [1665-1683] |
| 126 | UGCAGAGAUAUCCAGUCCU | AGGACUGGAUAUCUCUGCA | Chmp | [1626-1644] |
| 127 | GACUAUACUUUAUUUGGGU | ACCCAAAUAAAGUAUAGUC | Chmp | [1606-1624] |
| 128 | CCUGUUGGAUGACUGGAAA | UUUCCAGUCAUCCAACAGG | | [1571-1589] |
| 129 | GGAUUGUGUCUAAGCAGAG | CUCUGCUUAGACACAAUCC | Chmp | [396-414] |
| 130 | AGGAUUGUGUCUAAGCAGA | UCUGCUUAGACACAAUCCU | Chmp | [395-413] |
| 131 | AACUCCAUUUGCAUCAAUA | UAUUGAUGCAAAUGGAGUU | Chmp | [1544-1562] |
| 132 | CCAAGCUGUAUAUUGAUGG | CCAUCAAUAUACAGCUUGG | Chmp | [1453-1471] |
| 133 | ACUCCGAAAUUCUGCCCUU | AAGGGCAGAAUUUCGGAGU | Chmp | [1414-1432] |
| 134 | CUGGACAGAACGAUUUCGA | UCGAAAUCGUUCUGUCCAG | Chmp | [1367-1385] |
| 135 | GCCGAAAGACUUUACAGGU | ACCUGUAAAGUCUUUCGGC | Chmp | [1125-1143] |
| 136 | CGGCUGCAUCAGUCUUAAC | GUUAAGACUGAUGCAGCCG | Chmp | [911-929] |
| 137 | UCUACAUGCUGCUGACGUU | AACGUCAGCAGCAUGUAGA | Chmp | [838-856] |
| 138 | UCUUCUGGUAUACUCAUAA | UUAUGAGUAUACCAGAAGA | Chmp | [805-823] |
| 139 | GUGGUGUUACUAUCUGUAU | AUACAGAUAGUAACACCAC | Chmp | [562-580] |
| 140 | CCUGUGGUGUUACUAUCUG | CAGAUAGUAACACCACAGG | Chmp | [559-577] |
| 141 | CAGGAGAUUGUUGGAUAAA | UUUAUCCAACAAUCUCCUG | Chmp | [521-539] |
| 142 | UGGUUGAACAAAUGCAAGA | UCUUGCAUUUGUUCAACCA | Chmp | [2337-2355] |
| 143 | CCCUGAAGAAUAUUUGAUG | CAUCAAAUAUUCUUCAGGG | Chmp, Rat | [2172-2190] |
| 144 | ACCCUGAAGAAUAUUUGAU | AUCAAAUAUUCUUCAGGGU | Chmp | [2171-2189] |
| 145 | AGAGACCCUGAAGAAUAUU | AAUAUUCUUCAGGGUCUCU | Chmp | [2167-2185] |
| 146 | CUGUGUUAUGCCAAAGAAU | AUUCUUUGGCAUAACACAG | Chmp | [2057-2075] |
| 147 | GAGUGCAAUUUCUAAGACU | AGUCUUAGAAAUUGCACUC | Chmp | [2014-2032] |
| 148 | UGAAAACUUUUGCCAUGAA | UUCAUGGCAAAAGUUUUCA | Chmp | [1974-1992]+ |
| 149 | GAACAACUCAUAUGGGACA | UGUCCCAUAUGAGUUGUUC | Rat, Ms | [1925-1943] |
| 150 | AGAGGAAAAACAGUUGGUG | CACCAACUGUUUUUCCUCU | | [1851-1869] |
| 151 | CGGUGGAAACUUUUGUUUG | CAAACAAAAGUUUCCACCG | Chmp | [1812-1830] |
| 152 | CAGCAUCUGUUCUUAACCU | AGGUUAAGAACAGAUGCUG | Chmp | [418-436] |
| 153 | UAACAAGUUUUGGCAAGAG | CUCUUGCCAAAACUUGUUA | Chmp | [1679-1697] |
| 154 | UGCAUAACAAGUUUUGGCA | UGCCAAAACUUGUUAUGCA | Chmp, Rat, Ms | [1675-1693] |
| 155 | UUACUCUGUAUGUUGCAUA | UAUGCAACAUACAGAGUAA | Chmp | [1662-1680] |
| 156 | UGUAUAUUGAUGGUCCUUU | AAAGGACCAUCAAUAUACA | Chmp | [1459-1477] |
| 157 | CGAUUUCGAGAUUUACUAC | GUAGUAAAUCUCGAAAUCG | Chmp | [1377-1395] |
| 158 | GGACAGAACGAUUUCGAGA | UCUCGAAAUCGUUCUGUCC | Chmp | [1369-1387] |
| 159 | CCCUCAGAUGUCAUGGAAA | UUUCCAUGACAUCUGAGGG | Chmp | [1191-1209] |
| 160 | CCUUCUUGCUGUAUAACCA | UGGUUAUACAGCAAGAAGG | Chmp | [337-355] |
| 161 | CAUGAACCUUUCCCUGAAG | CUUCAGGGAAAGGUUCAUG | Chmp | [981-999] |
| 162 | CCAGAGUAUUUCUCAGAAC | GUUCUGAGAAAUACUCUGG | Chmp | [957-975] |
| 163 | CCAGCUCUCAGAAUAUUUC | GAAAUAUUCUGAGAGCUGG | Chmp | [934-952] |
| 164 | GACGUUGCAUGUUUCAGGA | UCCUGAAACAUGCAACGUC | Chmp | [851-869] |
| 165 | CUAUGAUAUCUUCUGGUAU | AUACCAGAAGAUAUCAUAG | Chmp | [797-815] |
| 166 | UGAAUGUCCUGCUUUUCUG | CAGAAAAGCAGGACAUUCA | Chmp | [313-331] |
| 167 | CUAACUAUGAUAUCUUCUG | CAGAAGAUAUCAUAGUUAG | Chmp | [793-811] |
| 168 | GCAAUAAGAGUUUCUAACU | AGUUAGAAACUCUUAUUGC | Chmp | [780-798] |
| 169 | UGAUCACAGCCUCUACAUA | UAUGUAGAGGCUGUGAUCA | Chmp | [760-778] |
| 170 | GAGGAUCCUAGAAAACUUC | GAAGUUUUCUAGGAUCCUC | Chmp | [684-702] |
| 171 | UUACAGUGAAGACUUUGUU | AACAAAGUCUUCACUGUAA | | [638-656] |
| 172 | ACAUUCCAUAUUACCUGUG | CACAGGUAAUAUGGAAUGU | Chmp | [546-564] |
| 173 | GAACAUUCCAUAUUACCUG | CAGGUAAUAUGGAAUGUUC | Chmp | [544-562] |
| 174 | GUGUUCCUCUUUAAAUUGU | ACAAUUUAAAGAGGAACAC | Chmp | [2315-2333] |
| 175 | GUCGUGUUCCUCUUUAAAU | AUUUAAAGAGGAACACGAC | Chmp | [2312-2330] |
| 176 | GGCUGAUUGUCGUGUUCCU | AGGAACACGACAAUCAGCC | Chmp | [2304-2322] |
| 177 | CACUGUUGAUUUUCAUGGU | ACCAUGAAAAUCAACAGUG | | [2240-2258] |
| 178 | UGCACACUGUUGAUUUUCA | UGAAAAUCAACAGUGUGCA | | [2236-2254] |
| 179 | AGACCCUGAAGAAUAUUUG | CAAAUAUUCUUCAGGGUCU | Chmp | [2169-2187] |
| 180 | GCAAGAUGACCGUCACAUU | AAUGUGACGGUCAUCUUGC | | [2126-2144] |
| 181 | UGCAGCCUUAUCCUUUUAC | GUAAAAGGAUAAGGCUGCA | Chmp | [441-459] |
| 182 | ACUGCAGCCUUAUCCUUUU | AAAAGGAUAAGGCUGCAGU | Chmp | [439-457] |
| 183 | GUGCAAUUUCUAAGACUUU | AAAGUCUUAGAAAUUGCAC | Chmp | [2016-2034] |
| 184 | AGUGCAAUUUCUAAGACUU | AAGUCUUAGAAAUUGCACU | Chmp, Rat, Ms | [2015-2033] |
| 185 | UGAAGCAGGACUCUAAAGA | UCUUUAGAGUCCUGCUUCA | Chmp | [1989-2007] |
| 186 | AAAGAGUCUUUCAGCUGAA | UUCAGCUGAAAGACUCUUU | Chmp | [1959-1977]+ |
| 187 | UCAUAUGGGACAAGAUUUG | CAAAUCUUGUCCCAUAUGA | | [1932-1950] |
| 188 | AACAACUCAUAUGGGACAA | UUGUCCCAUAUGAGUUGUU | Rat, Ms | [1926-1944] |
| 189 | CUAUCCAAGACUCUUCAUA | UAUGAAGAGUCUUGGAUAG | Chmp | [1893-1911] |
| 190 | UCACUAUCCAAGACUCUUC | GAAGAGUCUUGGAUAGUGA | Chmp | [1890-1908] |
| 191 | CAGAGGAAAAACAGUUGGU | ACCAACUGUUUUUCCUCUG | | [1850-1868] |
| 192 | AGCAUCUGUUCUUAACCUC | GAGGUUAAGAACAGAUGCU | Chmp | [419-437] |
| 193 | CCUCGGUGGAAACUUUUGU | ACAAAAGUUUCCACCGAGG | Chmp | [1809-1827] |
| 194 | AGAUGGGAUACAGAAGAUA | UAUCUUCUGUAUCCCAUCU | Chmp, Rat | [1742-1760] |
| 195 | UACUCUGUAUGUUGCAUAA | UUAUGCAACAUACAGAGUA | Chmp | [1663-1681] |
| 196 | AGACUAUACUUUAUUUGGG | CCCAAAUAAAGUAUAGUCU | Chmp | [1605-1623] |
| 197 | GCUGGAGGCAUUGGAGUAA | UUACUCCAAUGCCUCCAGC | Chmp | [1527-1545] |
| 198 | UCCCAAGCUGUAUAUUGAU | AUCAAUAUACAGCUUGGGA | Chmp | [1451-1469] |
| 199 | UCCAACUGAAACCAAAGCA | UGCUUUGGUUUCAGUUGGA | Chmp | [1316-1334] |
| 200 | CUGGUCAGUAUAUUACUCU | AGAGUAAUAUACUGACCAG | Chmp | [1243-1261] |
| 201 | CCAUCAUUUCGGUCAUAAG | CUUAUGACCGAAAUGAUGG | Chmp | [1168-1186] |
| 202 | GGUAUAUCCGGAGCAAUAA | UUAUUGCUCCGGAUAUACC | Chmp | [1141-1159] |
| 203 | GGAUUUCUGGACCUUUGUG | CACAAAGGUCCAGAAAUCC | Chmp, Rat, Ms | [1096-1114] |
| 204 | AAAACCUUCUUGCUGUAUA | UAUACAGCAAGAAGGUUUU | Chmp | [333-351] |
| 205 | AUACUCAUAACCUCUUCUU | AAGAAGAGGUUAUGAGUAU | Chmp | [814-832] |
| 206 | GUAUACUCAUAACCUCUUC | GAAGAGGUUAUGAGUAUAC | Chmp | [812-830] |
| 207 | CUUCUGGUAUACUCAUAAC | GUUAUGAGUAUACCAGAAG | Chmp | [806-824] |
| 208 | AUCUUCUGGUAUACUCAUA | UAUGAGUAUACCAGAAGAU | Chmp | [804-822] |
| 209 | UCCAUGAAUGUCCUGCUUU | AAAGCAGGACAUUCAUGGA | Chmp | [309-327] |
| 210 | GUGAAGACUUUGUUGAACU | AGUUCAACAAAGUCUUCAC | | [643-661] |
| 211 | ACAGUGAAGACUUUGUUGA | UCAACAAAGUCUUCACUGU | | [640-658] |
| 212 | ACAAAUGCAAGAUUGAACA | UGUUCAAUCUUGCAUUUGU | Chmp | [2344-2362] |
| 213 | UUGAUUUUCAUGGUGGAUU | AAUCCACCAUGAAAAUCAA | | [2245-2263] |
| 214 | AGAUGCACACUGUUGAUUU | AAAUCAACAGUGUGCAUCU | Chmp, Rat | [2233-2251] |
| 215 | ACCUCAACUGCAGCCUUAU | AUAAGGCUGCAGUUGAGGU | Chmp | [433-451] |
| 216 | CUCAUAUGGGACAAGAUUU | AAAUCUUGUCCCAUAUGAG | | [1931-1949] |
| 217 | CAGAACAACUCAUAUGGGA | UCCCAUAUGAGUUGUUCUG | | [1923-1941] |
| 218 | CCAGAACAACUCAUAUGGG | CCCAUAUGAGUUGUUCUGG | | [1922-1940] |
| 219 | CAAGACUCUUCAUAAACUG | CAGUUUAUGAAGAGUCUUG | Chmp | [1898-1916] |
| 220 | GGUGUUUUCUGUUGUGGAC | GUCCACAACAGAAAACACC | Chmp | [1866-1884] |
| 221 | CAGUUGGUGUUUUCUGUUG | CAACAGAAAACACCAACUG | Chmp | [1861-1879] |
| 222 | UCAAGACUGUUUAUAGGAC | GUCCUAUAAACAGUCUUGA | | [1788-1806] |
| 223 | CACUGAAUUCAAGACUGUU | AACAGUCUUGAAUUCAGUG | Chmp | [1780-1798] |
| 224 | GGAUACAGAAGAUAAUUGG | CCAAUUAUCUUCUGUAUCC | Chmp, Rat | [1747-1765] |
| 225 | AUGGGAUACAGAAGAUAAU | AUUAUCUUCUGUAUCCCAU | Chmp, Rat | [1744-1762] |
| 226 | AGAUUUACUCUGUAUGUUG | CAACAUACAGAGUAAAUCU | Chmp | [1658-1676] |
| 227 | CUGUAUAUUGAUGGUCCUU | AAGGACCAUCAAUAUACAG | Chmp | [1458-1476] |
| 228 | UCGAGAUUUACUACUGCCU | AGGCAGUAGUAAAUCUCGA | Chmp | [1382-1400] |
| 229 | GUAUCUGCAUUAGAAAAUC | GAUUUUCUAAUGCAGAUAC | Chmp | [1275-1293] |
| 230 | CCUGGUCAGUAUAUUACUC | GAGUAAUAUACUGACCAGG | Chmp | [1242-1260] |
| 231 | GACUUGGCUUUGGAUUUCU | AGAAAUCCAAAGCCAAGUC | Chmp | [1085-1103] |
| 232 | UGAUAUCUUCUGGUAUACU | AGUAUACCAGAAGAUAUCA | Chmp, Rat, Ms | [800-818] |
| 233 | AAGACUUUGUUGAACUGAA | UUCAGUUCAACAAAGUCUU | | [646-664] |
| 234 | UGAAGACUUUGUUGAACUG | CAGUUCAACAAAGUCUUCA | | [644-662] |
| 235 | CAGUGAAGACUUUGUUGAA | UUCAACAAAGUCUUCACUG | | [641-659] |
| 236 | UGUGGUGUUACUAUCUGUA | UACAGAUAGUAACACCACA | Chmp | [561-579] |
| 237 | CUGUGGUGUUACUAUCUGU | ACAGAUAGUAACACCACAG | Chmp | [560-578] |
| 238 | UGAACAAAUGCAAGAUUGA | UCAAUCUUGCAUUUGUUCA | Chmp | [2341-2359] |
| 239 | UUGUCGUGUUCCUCUUUAA | UUAAAGAGGAACACGACAA | Chmp | [2310-2328] |
| 240 | UGAUUGUCGUGUUCCUCUU | AAGAGGAACACGACAAUCA | Chmp | [2307-2325] |
| 241 | ACUGUUGAUUUUCAUGGUG | CACCAUGAAAAUCAACAGU | | [2241-2259] |
| 242 | CCUGAAGAAUAUUUGAUGU | ACAUCAAAUAUUCUUCAGG | Chmp, Rat | [2173-2191] |
| 243 | GGAAAUGUGAGAAUGUGGC | GCCACAUUCUCACAUUUCC | Chmp | [2109-2127] |
| 244 | CAACUGCAGCCUUAUCCUU | AAGGAUAAGGCUGCAGUUG | Chmp | [437-455] |
| 245 | UGCAAUUUCUAAGACUUUG | CAAAGUCUUAGAAAUUGCA | Chmp | [2017-2035] |
| 246 | GCUGAAAACUUUUGCCAUG | CAUGGCAAAAGUUUUCAGC | Chmp | [1972-1990]+ |
| 247 | AUAUGGGACAAGAUUUGAA | UUCAAAUCUUGUCCCAUAU | | [1934-1952] |
| 248 | AUCCAAGACUCUUCAUAAA | UUUAUGAAGAGUCUUGGAU | Chmp | [1895-1913] |
| 249 | CACUAUCCAAGACUCUUCA | UGAAGAGUCUUGGAUAGUG | Chmp | [1891-1909] |
| 250 | UAACAGAGGAAAAACAGUU | AACUGUUUUUCCUCUGUUA | | [1847-1865] |
| 251 | UGGGAUACAGAAGAUAAUU | AAUUAUCUUCUGUAUCCCA | Chmp, Rat | [1745-1763] |
| 252 | CAGUCAAACAGAUGGGAUA | UAUCCCAUCUGUUUGACUG | Chmp, Rat | [1733-1751] |
| 253 | GGGUAUGCAGAGAUAUCCA | UGGAUAUCUCUGCAUACCC | Chmp | [1621-1639] |
| 254 | UGGAGGCAUUGGAGUAACU | AGUUACUCCAAUGCCUCCA | Chmp | [1529-1547] |
| 255 | CCAUUUGAGGAAUCACUGA | UCAGUGAUUCCUCAAAUGG | Chmp | [1485-1503] |
| 256 | GACCUGGUCAGUAUAUUAC | GUAAUAUACUGACCAGGUC | Chmp | [1240-1258] |
| 257 | GGAAAUCCGAAUGGUCAAA | UUUGACCAUUCGGAUUUCC | Chmp | [1205-1223] |
| 258 | UCACCAUCAUUUCGGUCAU | AUGACCGAAAUGAUGGUGA | Chmp | [1165-1183] |
| 259 | CAGACUUGGCUUUGGAUUU | AAAUCCAAAGCCAAGUCUG | Chmp | [1083-1101] |
| 260 | ACAGACUUGGCUUUGGAUU | AAUCCAAAGCCAAGUCUGU | Chmp | [1082-1100] |
| 261 | GCUAAUUUUCCACAGACUU | AAGUCUGUGGAAAAUUAGC | Chmp | [1071-1089] |
| 262 | UGUGAAGAUUUGUAUGGAA | UUCCAUACAAAUCUUCACA | Chmp | [1037-1055] |
| 263 | GAAAACCUUCUUGCUGUAU | AUACAGCAAGAAGGUUUUC | Chmp | [332-350] |
| 264 | CUCAUAACCUCUUCUUUGU | ACAAAGAAGAGGUUAUGAG | Chmp | [817-835] |
| 265 | UGCAAUAAGAGUUUCUAAC | GUUAGAAACUCUUAUUGCA | Chmp | [779-797] |
| 266 | AAAAGCAGAACAUUCCAUA | UAUGGAAUGUUCUGCUUUU | Chmp | [537-555] |
| 267 | UAAAAGCAGAACAUUCCAU | AUGGAAUGUUCUGCUUUUA | Chmp | [536-554] |
| 268 | CGUGUUCCUCUUUAAAUUG | CAAUUUAAAGAGGAACACG | Chmp | [2314-2332] |
| 269 | UAAGGCUGAUUGUCGUGUU | AACACGACAAUCAGCCUUA | Chmp | [2301-2319] |
| 270 | UCAUGGUGGAUUCAAGAAC | GUUCUUGAAUCCACCAUGA | | [2252-2270] |
| 271 | CUGAAGAAUAUUUGAUGUG | CACAUCAAAUAUUCUUCAG | Chmp, Rat | [2174-2192] |
| 272 | CUUUGAAACUCAGCGGAAU | AUUCCGCUGAGUUUCAAAG | Chmp | [2031-2049] |
| 273 | AGUAACCAGAACAACUCAU | AUGAGUUGUUCUGGUUACU | | [1917-1935] |
| 274 | GGUAUGCAGAGAUAUCCAG | CUGGAUAUCUCUGCAUACC | Chmp | [1622-1640] |
| 275 | GGAGGCAUUGGAGUAACUC | GAGUUACUCCAAUGCCUCC | Chmp | [1530-1548] |
| 276 | CUGGAGGCAUUGGAGUAAC | GUUACUCCAAUGCCUCCAG | Chmp | [1528-1546] |
| 277 | CAUUUGAGGAAUCACUGAA | UUCAGUGAUUCCUCAAAUG | Chmp | [1486-1504] |
| 278 | GUGUAUCUGCAUUAGAAAA | UUUUCUAAUGCAGAUACAC | Chmp | [1273-1291] |
| 279 | AGCAAGACCUGGUCAGUAU | AUACUGACCAGGUCUUGCU | Chmp | [1235-1253] |
| 280 | CACAGACUUGGCUUUGGAU | AUCCAAAGCCAAGUCUGUG | Chmp | [1081-1099] |
| 281 | ACCAGCUCUCAGAAUAUUU | AAAUAUUCUGAGAGCUGGU | Chmp | [933-951] |
| 282 | AGAUGACCGUCACAUUACA | UGUAAUGUGACGGUCAUCU | | [2129-2147] |
| 283 | CAAGAUGACCGUCACAUUA | UAAUGUGACGGUCAUCUUG | | [2127-2145] |
| 284 | GACUUUGAAACUCAGCGGA | UCCGCUGAGUUUCAAAGUC | Chmp | [2029-2047] |
| 285 | CUGAAAACUUUUGCCAUGA | UCAUGGCAAAAGUUUUCAG | Chmp | [1973-1991]+ |
| 286 | GUUGGUGUUUUCUGUUGUG | CACAACAGAAAACACCAAC | Chmp | [1863-1881] |
| 287 | ACAGAUGGGAUACAGAAGA | UCUUCUGUAUCCCAUCUGU | Chmp, Rat | [1740-1758] |
| 288 | GUCAAACAGAUGGGAUACA | UGUAUCCCAUCUGUUUGAC | Chmp, Rat | [1735-1753] |
| 289 | GGCUUCCACUCAGUCUUUG | CAAAGACUGAGUGGAAGCC | | [104-122] |
| 290 | CUAGUCAAGACUCCGAAAU | AUUUCGGAGUCUUGACUAG | Chmp | [1405-1423] |
| 291 | AGCUCUCAGAAUAUUUCCU | AGGAAAUAUUCUGAGAGCU | Chmp | [936-954] |
| 292 | AACCAGCUCUCAGAAUAUU | AAUAUUCUGAGAGCUGGUU | Chmp | [932-950] |
| 293 | AUAACCUCUUCUUUGUCUU | AAGACAAAGAAGAGGUUAU | Chmp | [820-838] |
| 294 | CACAGAAGGUUCCAAGCAG | CUGCUUGGAACCUUCUGUG | Chmp | [496-514] |
| 295 | GAAAACUUUUGCCAUGAAG | CUUCAUGGCAAAAGUUUUC | Chmp | [1975-1993]+ |
| 296 | AGCAAAAUAUAACAGAGGA | UCCUCUGUUAUAUUUUGCU | | [1838-1856] |
| 297 | UCAAACAGAUGGGAUACAG | CUGUAUCCCAUCUGUUUGA | Chmp, Rat | [1736-1754] |
| 298 | UCAGUCAAACAGAUGGGAU | AUCCCAUCUGUUUGACUGA | Chmp, Rat, Ms | [1732-1750] |
| 299 | CCCUCACAAUGUGUCCAAC | GUUGGACACAUUGUGAGGG | Chmp | [1303-1321] |
| 300 | CCAUUUACCCUCACAAUGU | ACAUUGUGAGGGUAAAUGG | Chmp | [1296-1314] |
| 301 | AGACUUUACAGGUAUAUCC | GGAUAUACCUGUAAAGUCU | Chmp | [1131-1149] |
| 302 | CAGAGUAUUUCUCAGAACA | UGUUCUGAGAAAUACUCUG | Chmp | [958-976] |
| 303 | AGAAAACUGUUAGAUGCAC | GUGCAUCUAACAGUUUUCU | Chmp | [2222-2240] |
| 304 | GCCUCCAUCUAGUCAAGAC | GUCUUGACUAGAUGGAGGC | Chmp | [1397-1415] |
| 305 | AUAAGCCAGUCACCAUCAU | AUGAUGGUGACUGGCUUAU | Chmp | [1156-1174] |
| 306 | GGAGCAAUAAGCCAGUCAC | GUGACUGGCUUAUUGCUCC | Chmp | [1150-1168] |
| 307 | ACCUUUCCCUGAAGGAUUU | AAAUCCUUCAGGGAAAGGU | Chmp | [986-1004] |
| 308 | ACCUCUUCUUUGUCUUCUA | UAGAAGACAAAGAAGAGGU | Chmp, Rat, Ms | [823-841] |
| 309 | CCGAAAUUCUGCCCUUCAU | AUGAAGGGCAGAAUUUCGG | Chmp | [1417-1435] |
| 310 | AAGCCAGUCACCAUCAUUU | AAAUGAUGGUGACUGGCUU | Chmp | [1158-1176] |
| 311 | CGGAGCAAUAAGCCAGUCA | UGACUGGCUUAUUGCUCCG | Chmp | [1149-1167] |
| 312 | GUAUGGAAGAGCCCAGAUU | AAUCUGGGCUCUUCCAUAC | Chmp | [1048-1066] |
| 313 | CCCUGAAGGAUUUUCAAAA | UUUUGAAAAUCCUUCAGGG | Chmp | [992-1010] |
| 314 | CCCUCAACUUCUCAGUGAA | UUCACUGAGAAGUUGAGGG | Chmp | [619-637] |
| 315 | CCAUGUGCCGAACACUCUU | AAGAGUGUUCGGCACAUGG | | [460-478] |
| 316 | GGACCCAAUUCACUAUCCA | UGGAUAGUGAAUUGGGUCC | Chmp | [1881-1899] |
| 317 | GCAUUGGAGUAACUCCAUU | AAUGGAGUUACUCCAAUGC | Chmp | [1534-1552] |
| 318 | CCUUCAUUCAAUCUAGAAA | UUUCUAGAUUGAAUGAAGG | Chmp, Rat | [1429-1447] |
| 319 | GGCCAGAGUAUCACUACCU | AGGUAGUGAUACUCUGGCC | Chmp | [358-376] |
| 320 | CUCACAAUGUGUCCAACUG | CAGUUGGACACAUUGUGAG | Chmp | [1305-1323] |
| 321 | CCAGUCACCAUCAUUUCGG | CCGAAAUGAUGGUGACUGG | Chmp | [1161-1179] |
| 322 | CCAAGCUAAUUUUCCACAG | CUGUGGAAAAUUAGCUUGG | Chmp | [1067-1085] |
| 323 | ACCAGAGUAUUUCUCAGAA | UUCUGAGAAAUACUCUGGU | Chmp | [956-974] |
| 324 | GCCUCUACAUAUGCAAUAA | UUAUUGCAUAUGUAGAGGC | Chmp | [768-786] |
| 325 | UGGUGGUGCUAUUCCUCAU | AUGAGGAAUAGCACCACCA | Chmp | [742-760] |
| 326 | GGAUAAAAGCAGAACAUUC | GAAUGUUCUGCUUUUAUCC | Chmp | [533-551] |
| 327 | UGGAUAAAAGCAGAACAUU | AAUGUUCUGCUUUUAUCCA | Chmp | [532-550] |
| 328 | UUGGAUAAAAGCAGAACAU | AUGUUCUGCUUUUAUCCAA | Chmp | [531-549] |
| 329 | GCCUCAGCAUCUGUUCUUA | UAAGAACAGAUGCUGAGGC | Chmp | [414-432] |
| 330 | GUAUGUUGCAUAACAAGUU | AACUUGUUAUGCAACAUAC | Chmp | [1669-1687] |
| 331 | UCCUUUUGGAAGUCCAUUU | AAAUGGACUUCCAAAAGGA | Chmp | [1472-1490] |
| 332 | CCUCCAUCUAGUCAAGACU | AGUCUUGACUAGAUGGAGG | Chmp | [1398-1416] |
| 333 | GCCAGAGUAUCACUACCUC | GAGGUAGUGAUACUCUGGC | Chmp | [359-377] |
| 334 | CCUCACAAUGUGUCCAACU | AGUUGGACACAUUGUGAGG | Chmp | [1304-1322] |
| 335 | GCAUUAGAAAAUCAUCCAU | AUGGAUGAUUUUCUAAUGC | Chmp | [1281-1299] |
| 336 | GAAAUCCGAAUGGUCAAAG | CUUUGACCAUUCGGAUUUC | Chmp | [1206-1224] |
| 337 | CAUAAGUCAUCCCUCAGAU | AUCUGAGGGAUGACUUAUG | Chmp | [1181-1199] |
| 338 | UCAUAAGUCAUCCCUCAGA | UCUGAGGGAUGACUUAUGA | Chmp | [1180-1198] |
| 339 | GUCACCAUCAUUUCGGUCA | UGACCGAAAUGAUGGUGAC | Chmp | [1164-1182] |
| 340 | CAAUAAGCCAGUCACCAUC | GAUGGUGACUGGCUUAUUG | Chmp | [1154-1172] |
| 341 | AGAGUAUUUCUCAGAACAU | AUGUUCUGAGAAAUACUCU | Chmp | [959-977] |
| 342 | GCAUCAGUCUUAACCGAAC | GUUCGGUUAAGACUGAUGC | Chmp | [916-934] |
| 343 | UGCUGAAGUAUCAAACUAA | UUAGUUUGAUACUUCAGCA | Chmp | [874-892] |
| 344 | UGGUGCUAUUCCUCAUGAU | AUCAUGAGGAAUAGCACCA | Chmp | [745-763] |
| 345 | GGUGGUGCUAUUCCUCAUG | CAUGAGGAAUAGCACCACC | Chmp | [743-761] |
| 346 | CUAGAAAACUUCUCUUCAC | GUGAAGAGAAGUUUUCUAG | Chmp | [691-709] |
| 347 | CUCAACUUCUCAGUGAAUU | AAUUCACUGAGAAGUUGAG | Chmp | [621-639] |
| 348 | CCAUAUUACCUGUGGUGUU | AACACCACAGGUAAUAUGG | Chmp | [551-569] |
| 349 | CCGUCACAUUACAUGUUUA | UAAACAUGUAAUGUGACGG | | [2135-2153] |
| 350 | GCAAGACCUGGUCAGUAUA | UAUACUGACCAGGUCUUGC | Chmp | [1236-1254] |
| 351 | AGCUAAUUUUCCACAGACU | AGUCUGUGGAAAAUUAGCU | Chmp | [1070-1088] |
| 352 | CAAGCUAAUUUUCCACAGA | UCUGUGGAAAAUUAGCUUG | Chmp | [1068-1086] |
| 353 | GAAGAUUUGUAUGGAAGAG | CUCUUCCAUACAAAUCUUC | Chmp | [1040-1058] |
| 354 | GGGCUGCUGAAGUAUCAAA | UUUGAUACUUCAGCAGCCC | Chmp | [870-888] |
| 355 | CCUCUUCUUUGUCUUCUAC | GUAGAAGACAAAGAAGAGG | Chmp, Rat, Ms | [824-842] |
| 356 | UCAACUUCUCAGUGAAUUA | UAAUUCACUGAGAAGUUGA | Chmp | [622-640] |
| 357 | UUGUUGGAUAAAAGCAGAA | UUCUGCUUUUAUCCAACAA | Chmp | [528-546] |
| 358 | ACCUCUGCCUGUUCAUCUG | CAGAUGAACAGGCAGAGGU | | [286-304] |
| 359 | AGCUGAACUUGCUCAAUCU | AGAUUGAGCAAGUUCAGCU | Chmp | [2283-2301] |
| 360 | AGGAGCUGAACUUGCUCAA | UUGAGCAAGUUCAGCUCCU | Chmp | [2280-2298] |
| 361 | GGAAACCAAAGAGACCCUG | CAGGGUCUCUUUGGUUUCC | Chmp | [2158-2176] |
| 362 | CUGUUGUGGACCCAAUUCA | UGAAUUGGGUCCACAACAG | Chmp | [1874-1892] |
| 363 | GACCUGACUAUGUCAACAU | AUGUUGACAUAGUCAGGUC | Chmp | [1702-1720] |
| 364 | UCCAUUUGCAUCAAUACUC | GAGUAUUGAUGCAAAUGGA | Chmp | [1547-1565] |
| 365 | CCUUUUGGAAGUCCAUUUG | CAAAUGGACUUCCAAAAGG | Chmp | [1473-1491] |
| 366 | CGAAAUUCUGCCCUUCAUU | AAUGAAGGGCAGAAUUUCG | Chmp | [1418-1436] |
| 367 | CCCAGUGUAUCUGCAUUAG | CUAAUGCAGAUACACUGGG | Chmp | [1269-1287] |
| 368 | CAGAGUUUACCCAGCACAA | UUGUGCUGGGUAAACUCUG | Chmp | [1015-1033] |
| 369 | GCAGAGUUUACCCAGCACA | UGUGCUGGGUAAACUCUGC | Chmp | [1014-1032] |
| 370 | UCAAAACCGGCAGAGUUUA | UAAACUCUGCCGGUUUUGA | Chmp | [1005-1023] |
| 371 | CCUGAAGGAUUUUCAAAAC | GUUUUGAAAAUCCUUCAGG | Chmp | [993-1011] |
| 372 | CUCAGAACAUUUUCAUGAA | UUCAUGAAAAUGUUCUGAG | Chmp | [968-986] |
| 373 | UCUUCUUUGUCUUCUACAU | AUGUAGAAGACAAAGAAGA | Chmp, Rat, Ms | [826-844] |
| 374 | AGCCUCUACAUAUGCAAUA | UAUUGCAUAUGUAGAGGCU | Chmp | [767-785] |
| 375 | CCUCAACUUCUCAGUGAAU | AUUCACUGAGAAGUUGAGG | Chmp | [620-638] |
| 376 | GGAGAACCAGGAGAUUGUU | AACAAUCUCCUGGUUCUCC | Chmp | [514-532] |
| 377 | AAACCAAAGAGACCCUGAA | UUCAGGGUCUCUUUGGUUU | Chmp | [2160-2178] |
| 378 | CGUCACAUUACAUGUUUAA | UUAAACAUGUAAUGUGACG | | [2136-2154] |
| 379 | GGAAAAACAGUUGGUGUUU | AAACACCAACUGUUUUUCC | | [1854-1872] |
| 380 | UGCAGAUUUACUCUGUAUG | CAUACAGAGUAAAUCUGCA | Chmp | [1655-1673] |
| 381 | UUGCAGAUUUACUCUGUAU | AUACAGAGUAAAUCUGCAA | Chmp | [1654-1672] |
| 382 | GUCCCAGUGUAUCUGCAUU | AAUGCAGAUACACUGGGAC | Chmp | [1267-1285] |
| 383 | CAAGACCUGGUCAGUAUAU | AUAUACUGACCAGGUCUUG | Chmp | [1237-1255] |
| 384 | GUAUAACCAAGGGCCAGAG | CUCUGGCCCUUGGUUAUAC | Chmp | [347-365] |
| 385 | CUGUGCCGAAAGACUUUAC | GUAAAGUCUUUCGGCACAG | Chmp | [1121-1139] |
| 386 | UGGCUUUGGAUUUCUGGAC | GUCCAGAAAUCCAAAGCCA | Chmp | [1089-1107] |
| 387 | GCCCAGAUUCCAAGCUAAU | AUUAGCUUGGAAUCUGGGC | Chmp | [1058-1076] |
| 388 | AGCCCAGAUUCCAAGCUAA | UUAGCUUGGAAUCUGGGCU | Chmp | [1057-1075] |
| 389 | AGCACAAAUUUGUGAAGAU | AUCUUCACAAAUUUGUGCU | Chmp | [1027-1045] |
| 390 | CCCAGCACAAAUUUGUGAA | UUCACAAAUUUGUGCUGGG | Chmp | [1024-1042] |
| 391 | CUCAGUGAAUUACAGUGAA | UUCACUGUAAUUCACUGAG | | [629-647] |
| 392 | GCAGGAGAACCAGGAGAUU | AAUCUCCUGGUUCUCCUGC | Chmp | [511-529] |
| 393 | GAACACUCUUGGCUUACCU | AGGUAAGCCAAGAGUGUUC | Chmp | [469-487] |
| 394 | GGAGCUGAACUUGCUCAAU | AUUGAGCAAGUUCAGCUCC | Chmp | [2281-2299] |
| 395 | UGGAAACCAAAGAGACCCU | AGGGUCUCUUUGGUUUCCA | Chmp | [2157-2175] |
| 396 | CCUCAGCAUCUGUUCUUAA | UUAAGAACAGAUGCUGAGG | Chmp | [415-433] |
| 397 | CCAGCUGUACCUCAGUCAA | UUGACUGAGGUACAGCUGG | Chmp, Rat, Ms | [1721-1739] |
| 398 | GCAGAUUUACUCUGUAUGU | ACAUACAGAGUAAAUCUGC | Chmp | [1656-1674] |
| 399 | UCCGUUGGUUUGCAGAUUU | AAAUCUGCAAACCAACGGA | Chmp | [1645-1663] |
| 400 | UGAUGGUCCUUUUGGAAGU | ACUUCCAAAAGGACCAUCA | Chmp | [1466-1484] |
| 401 | CUGAAACCAAAGCAACAUU | AAUGUUGCUUUGGUUUCAG | Chmp, Rat, Ms | [1321-1339] |
| 402 | GUGUCCAACUGAAACCAAA | UUUGGUUUCAGUUGGACAC | Chmp | [1313-1331] |
| 403 | CUUUGGAUUUCUGGACCUU | AAGGUCCAGAAAUCCAAAG | Chmp | [1092-1110] |
| 404 | GUGAAGAUUUGUAUGGAAG | CUUCCAUACAAAUCUUCAC | Chmp | [1038-1056] |
| 405 | GCACAAAUUUGUGAAGAUU | AAUCUUCACAAAUUUGUGC | Chmp | [1028-1046] |
| 406 | UCAGAACAUUUUCAUGAAC | GUUCAUGAAAAUGUUCUGA | Chmp | [969-987] |
| 407 | CUCUCAGAAUAUUUCCUUA | UAAGGAAAUAUUCUGAGAG | Chmp | [938-956] |
| 408 | CUGCUUUUCUGGAAAACCU | AGGUUUUCCAGAAAAGCAG | Chmp, Rat | [321-339] |
| 409 | GGCUGCUGAAGUAUCAAAC | GUUUGAUACUUCAGCAGCC | Chmp | [871-889] |
| 410 | GUCCUGCUUUUCUGGAAAA | UUUUCCAGAAAAGCAGGAC | Chmp, Rat | [318-336] |
| 411 | CCUCUACAUAUGCAAUAAG | CUUAUUGCAUAUGUAGAGG | Chmp | [769-787] |
| 412 | AUCUGGUGAAUGCCCUCAA | UUGAGGGCAUUCACCAGAU | Chmp | [607-625] |
| 413 | CGAGGAUCACAGAAGGUUC | GAACCUUCUGUGAUCCUCG | Chmp | [489-507] |
| 414 | CGAACACUCUUGGCUUACC | GGUAAGCCAAGAGUGUUCG | Chmp | [468-486] |
| 415 | GUUUUUGGUUGAACAAAUG | CAUUUGUUCAACCAAAAAC | Chmp | [2332-2350] |
| 416 | UGAACUUGCUCAAUCUAAG | CUUAGAUUGAGCAAGUUCA | Chmp | [2286-2304] |
| 417 | UCACUUUUCAGUUCUCAAA | UUUGAGAACUGAAAAGUGA | Chmp | [2198-2216] |
| 418 | ACCAAAGAGACCCUGAAGA | UCUUCAGGGUCUCUUUGGU | Chmp | [2162-2180] |
| 419 | ACCGUCACAUUACAUGUUU | AAACAUGUAAUGUGACGGU | | [2134-2152] |
| 420 | GCGGAAUCAAUCAGCUGUG | CACAGCUGAUUGAUUCCGC | Chmp | [2043-2061] |
| 421 | UGAAACUCAGCGGAAUCAA | UUGAUUCCGCUGAGUUUCA | Chmp | [2034-2052] |
| 422 | UUAAACACCUCUGCCUGUU | AACAGGCAGAGGUGUUUAA | | [280-298] |
| 423 | CUCAGCAUCUGUUCUUAAC | GUUAAGAACAGAUGCUGAG | Chmp | [416-434] |
| 424 | AGAGAACAGACCUGACUAU | AUAGUCAGGUCUGUUCUCU | Chmp | [1694-1712] |
| 425 | CAGAUUUACUCUGUAUGUU | AACAUACAGAGUAAAUCUG | Chmp | [1657-1675] |
| 426 | GUCCUUCCGUUGGUUUGCA | UGCAAACCAACGGAAGGAC | Chmp | [1640-1658] |
| 427 | AGCUUAGAAGACUAUACUU | AAGUAUAGUCUUCUAAGCU | Chmp | [1597-1615] |
| 428 | CCCUGUUGGAUGACUGGAA | UUCCAGUCAUCCAACAGGG | | [1570-1588] |
| 429 | GUCCUUUUGGAAGUCCAUU | AAUGGACUUCCAAAAGGAC | Chmp | [1471-1489] |
| 430 | ACUACCUCCACCAGAUGUU | AACAUCUGGUGGAGGUAGU | Chmp | [370-388] |
| 431 | CAGAGUAUCACUACCUCCA | UGGAGGUAGUGAUACUCUG | Chmp | [361-379] |
| 432 | GGACCUUUGUGCCUGUACU | AGUACAGGCACAAAGGUCC | Chmp | [1104-1122] |
| 433 | CUGGACCUUUGUGCCUGUA | UACAGGCACAAAGGUCCAG | Chmp | [1102-1120] |
| 434 | AAGCUAAUUUUCCACAGAC | GUCUGUGGAAAAUUAGCUU | Chmp | [1069-1087] |
| 435 | UCCAAGCUAAUUUUCCACA | UGUGGAAAAUUAGCUUGGA | Chmp | [1066-1084] |
| 436 | CCCAGAUUCCAAGCUAAUU | AAUUAGCUUGGAAUCUGGG | Chmp | [1059-1077] |
| 437 | GGAAGAGCCCAGAUUCCAA | UUGGAAUCUGGGCUCUUCC | Chmp | [1052-1070] |
| 438 | UGAAGAUUUGUAUGGAAGA | UCUUCCAUACAAAUCUUCA | Chmp | [1039-1057] |
| 439 | AGAGUUUACCCAGCACAAA | UUUGUGCUGGGUAAACUCU | Chmp | [1016-1034] |
| 440 | AUAUUUCCUUACCAGAGUA | UACUCUGGUAAGGAAAUAU | Chmp | [946-964] |
| 441 | GCUGCUGACGUUGCAUGUU | AACAUGCAACGUCAGCAGC | Chmp | [845-863] |
| 442 | CUGGUAUACUCAUAACCUC | GAGGUUAUGAGUAUACCAG | Chmp | [809-827] |
| 443 | CUCUACAUAUGCAAUAAGA | UCUUAUUGCAUAUGUAGAG | Chmp | [770-788] |
| 444 | UGCAGCAAGAUACCGAGAU | AUCUCGGUAUCUUGCUGCA | Chmp | [665-683] |
| 445 | UCAGUGAAUUACAGUGAAG | CUUCACUGUAAUUCACUGA | | [630-648] |
| 446 | CUGAACUUGCUCAAUCUAA | UUAGAUUGAGCAAGUUCAG | Chmp | [2285-2303] |
| 447 | GCUGAACUUGCUCAAUCUA | UAGAUUGAGCAAGUUCAGC | Chmp | [2284-2302] |
| 448 | UGUUUAAUCUGGAAACCAA | UUGGUUUCCAGAUUAAACA | Chmp | [2148-2166] |
| 449 | AACUGAGUAACCAGAACAA | UUGUUCUGGUUACUCAGUU | | [1912-1930] |
| 450 | CUCUUCAUAAACUGAGUAA | UUACUCAGUUUAUGAAGAG | Chmp | [1903-1921] |
| 451 | ACCUGACUAUGUCAACAUC | GAUGUUGACAUAGUCAGGU | Chmp | [1703-1721] |
| 452 | UCCUUCCGUUGGUUUGCAG | CUGCAAACCAACGGAAGGA | Chmp | [1641-1659] |
| 453 | GAUAUCCAGUCCUUCCGUU | AACGGAAGGACUGGAUAUC | Chmp | [1632-1650] |
| 454 | UGACUGGAAACCAUACAAG | CUUGUAUGGUUUCCAGUCA | Chmp, Rat, Ms | [1580-1598] |
| 455 | CUAGAAAUUAUCCCAAGCU | AGCUUGGGAUAAUUUCUAG | Chmp | [1441-1459] |
| 456 | UCCCAGUGUAUCUGCAUUA | UAAUGCAGAUACACUGGGA | Chmp | [1268-1286] |
| 457 | AAGACCUGGUCAGUAUAUU | AAUAUACUGACCAGGUCUU | Chmp | [1238-1256] |
| 458 | GGUCAUAAGUCAUCCCUCA | UGAGGGAUGACUUAUGACC | Chmp | [1178-1196] |
| 459 | UCGGUCAUAAGUCAUCCCU | AGGGAUGACUUAUGACCGA | Chmp | [1176-1194] |
| 460 | CAGUCACCAUCAUUUCGGU | ACCGAAAUGAUGGUGACUG | Chmp | [1162-1180] |
| 461 | UGCCUGUACUGUGCCGAAA | UUUCGGCACAGUACAGGCA | Chmp | [1113-1131] |
| 462 | AGAGCCCAGAUUCCAAGCU | AGCUUGGAAUCUGGGCUCU | Chmp | [1055-1073] |
| 463 | AGGAUUUUCAAAACCGGCA | UGCCGGUUUUGAAAAUCCU | Chmp | [998-1016] |
| 464 | AGAAAACUUCUCUUCACAA | UUGUGAAGAGAAGUUUUCU | Chmp | [693-711] |
| 465 | UGAAUGCCCUCAACUUCUC | GAGAAGUUGAGGGCAUUCA | Chmp | [613-631] |
| 466 | UGGUGAAUGCCCUCAACUU | AAGUUGAGGGCAUUCACCA | Chmp, Ms | [610-628] |
| 467 | UCAUCUGGCUCUCCAUGAA | UUCAUGGAGAGCCAGAUGA | Chmp | [298-316] |
| 468 | AGCAGGAGAACCAGGAGAU | AUCUCCUGGUUCUCCUGCU | Chmp | [510-528] |
| 469 | GAGCUGAACUUGCUCAAUC | GAUUGAGCAAGUUCAGCUC | Chmp | [2282-2300] |
| 470 | GGAUUCAAGAACUCCCUAG | CUAGGGAGUUCUUGAAUCC | Chmp | [2259-2277] |
| 471 | GUGGAUUCAAGAACUCCCU | AGGGAGUUCUUGAAUCCAC | Chmp | [2257-2275] |
| 472 | UGAUUCACUUUUCAGUUCU | AGAACUGAAAAGUGAAUCA | Chmp | [2194-2212] |
| 473 | GACCGUCACAUUACAUGUU | AACAUGUAAUGUGACGGUC | | [2133-2151] |
| 474 | UCAGCUGUGUUAUGCCAAA | UUUGGCAUAACACAGCUGA | Chmp | [2053-2071] |
| 475 | GUCUUUCAGCUGAAAACUU | AAGUUUUCAGCUGAAAGAC | Chmp | [1964-1982]+ |
| 476 | CAGCUGUACCUCAGUCAAA | UUUGACUGAGGUACAGCUG | Chmp, Rat, Ms | [1722-1740] |
| 477 | AUGUCAACAUCCAGCUGUA | UACAGCUGGAUGUUGACAU | Chmp | [1711-1729] |
| 478 | AGACCUGACUAUGUCAACA | UGUUGACAUAGUCAGGUCU | Chmp | [1701-1719] |
| 479 | CCAGUCCUUCCGUUGGUUU | AAACCAACGGAAGGACUGG | Chmp | [1637-1655] |
| 480 | UCAACACCCUGUUGGAUGA | UCAUCCAACAGGGUGUUGA | | [1564-1582] |
| 481 | GGAGUAACUCCAUUUGCAU | AUGCAAAUGGAGUUACUCC | Chmp | [1539-1557] |
| 482 | CAUUGGAGUAACUCCAUUU | AAAUGGAGUUACUCCAAUG | Chmp | [1535-1553] |
| 483 | GGGUUAAACACCUCUGCCU | AGGCAGAGGUGUUUAACCC | Ms | [277-295] |
| 484 | UGGAAGUCCAUUUGAGGAA | UUCCUCAAAUGGACUUCCA | Chmp | [1478-1496] |
| 485 | AUCACUACCUCCACCAGAU | AUCUGGUGGAGGUAGUGAU | Chmp | [367-385] |
| 486 | ACUGAAACCAAAGCAACAU | AUGUUGCUUUGGUUUCAGU | Chmp, Rat, Ms | [1320-1338] |
| 487 | UCACAAUGUGUCCAACUGA | UCAGUUGGACACAUUGUGA | Chmp | [1306-1324] |
| 488 | GUCAGUAUAUUACUCUACA | UGUAGAGUAAUAUACUGAC | Chmp | [1246-1264] |
| 489 | UCCGAAUGGUCAAAGAAAA | UUUUCUUUGACCAUUCGGA | Chmp | [1210-1228] |
| 490 | CGGUCAUAAGUCAUCCCUC | GAGGGAUGACUUAUGACCG | Chmp | [1177-1195] |
| 491 | CAGCACAAAUUUGUGAAGA | UCUUCACAAAUUUGUGCUG | Chmp | [1026-1044] |
| 492 | UGAAGGAUUUUCAAAACCG | CGGUUUUGAAAAUCCUUCA | Chmp | [995-1013] |
| 493 | CUGAAGGAUUUUCAAAACC | GGUUUUGAAAAUCCUUCAG | Chmp | [994-1012] |
| 494 | CCUUACCAGAGUAUUUCUC | GAGAAAUACUCUGGUAAGG | Chmp | [952-970] |
| 495 | CUCAGAAUAUUUCCUUACC | GGUAAGGAAAUAUUCUGAG | Chmp | [940-958] |
| 496 | GCUGCUGAAGUAUCAAACU | AGUUUGAUACUUCAGCAGC | Chmp | [872-890] |
| 497 | GGUAUACUCAUAACCUCUU | AAGAGGUUAUGAGUAUACC | Chmp | [811-829] |
| 498 | GCUCAAUCUAAGGCUGAUU | AAUCAGCCUUAGAUUGAGC | Chmp | [2293-2311] |
| 499 | GAUGUGAUGAUUCACUUUU | AAAAGUGAAUCAUCACAUC | Chmp | [2187-2205] |
| 500 | UGUGGACCCAAUUCACUAU | AUAGUGAAUUGGGUCCACA | Chmp | [1878-1896] |
| 501 | UCUGUAUGUUGCAUAACAA | UUGUUAUGCAACAUACAGA | Chmp | [1666-1684] |
| 502 | GGCAUUGGAGUAACUCCAU | AUGGAGUUACUCCAAUGCC | Chmp | [1533-1551] |
| 503 | GGUCCUUUUGGAAGUCCAU | AUGGACUUCCAAAAGGACC | Chmp | [1470-1488] |
| 504 | UGCAUUAGAAAAUCAUCCA | UGGAUGAUUUUCUAAUGCA | Chmp | [1280-1298] |
| 505 | CCAGUGUAUCUGCAUUAGA | UCUAAUGCAGAUACACUGG | Chmp | [1270-1288] |
| 506 | CUACAUUGUCCCAGUGUAU | AUACACUGGGACAAUGUAG | Chmp, Rat | [1260-1278] |
| 507 | GGUCAGUAUAUUACUCUAC | GUAGAGUAAUAUACUGACC | Chmp | [1245-1263] |
| 508 | CAGGUAUAUCCGGAGCAAU | AUUGCUCCGGAUAUACCUG | Chmp | [1139-1157] |
| 509 | GCUUUGGAUUUCUGGACCU | AGGUCCAGAAAUCCAAAGC | Chmp | [1091-1109] |
| 510 | CCAGCACAAAUUUGUGAAG | CUUCACAAAUUUGUGCUGG | Chmp | [1025-1043] |
| 511 | GCUCUCAGAAUAUUUCCUU | AAGGAAAUAUUCUGAGAGC | Chmp | [937-955] |
| 512 | UGCUGACGUUGCAUGUUUC | GAAACAUGCAACGUCAGCA | Chmp | [847-865] |
| 513 | ACAGCCUCUACAUAUGCAA | UUGCAUAUGUAGAGGCUGU | Chmp | [765-783] |
| 514 | UCCUAGAAAACUUCUCUUC | GAAGAGAAGUUUUCUAGGA | Chmp | [689-707] |
| 515 | GAACUGAAUGCAGCAAGAU | AUCUUGCUGCAUUCAGUUC | Chmp, Ms | [657-675] |
| 516 | CUAUCUGUAUUUUCUCAGG | CCUGAGAAAAUACAGAUAG | Chmp, Ms | [571-589] |
| 517 | UGCUCAAUCUAAGGCUGAU | AUCAGCCUUAGAUUGAGCA | Chmp | [2292-2310] |
| 518 | UCCCUAGUGAGGAGCUGAA | UUCAGCUCCUCACUAGGGA | Chmp | [2271-2289] |
| 519 | UGAUGUGAUGAUUCACUUU | AAAGUGAAUCAUCACAUCA | Chmp | [2186-2204] |
| 520 | GGCAAGAUGACCGUCACAU | AUGUGACGGUCAUCUUGCC | | [2125-2143] |
| 521 | AGCUGUGUUAUGCCAAAGA | UCUUUGGCAUAACACAGCU | Chmp | [2055-2073] |
| 522 | UGCACUGAAUUCAAGACUG | CAGUCUUGAAUUCAGUGCA | Chmp | [1778-1796] |
| 523 | AGCCUCAGCAUCUGUUCUU | AAGAACAGAUGCUGAGGCU | Chmp | [413-431] |
| 524 | AGCUGUACCUCAGUCAAAC | GUUUGACUGAGGUACAGCU | Chmp, Rat, Ms | [1723-1741] |
| 525 | ACAGACCUGACUAUGUCAA | UUGACAUAGUCAGGUCUGU | Chmp | [1699-1717] |
| 526 | UCCAGUCCUUCCGUUGGUU | AACCAACGGAAGGACUGGA | Chmp | [1636-1654] |
| 527 | CAAUCUAGAAAUUAUCCCA | UGGGAUAAUUUCUAGAUUG | Chmp | [1437-1455] |
| 528 | UCCGAAAUUCUGCCCUUCA | UGAAGGGCAGAAUUUCGGA | Chmp | [1416-1434] |
| 529 | CUCCGAAAUUCUGCCCUUC | GAAGGGCAGAAUUUCGGAG | Chmp | [1415-1433] |
| 530 | AGAUUUACUACUGCCUCCA | UGGAGGCAGUAGUAAAUCU | Chmp, Rat | [1385-1403] |
| 531 | CGAGAUUUACUACUGCCUC | GAGGCAGUAGUAAAUCUCG | Chmp, Rat | [1383-1401] |
| 532 | UCUACAUUGUCCCAGUGUA | UACACUGGGACAAUGUAGA | Chmp, Rat | [1259-1277] |
| 533 | UCAUGGAAAUCCGAAUGGU | ACCAUUCGGAUUUCCAUGA | Chmp | [1201-1219] |
| 534 | UCCCUCAGAUGUCAUGGAA | UUCCAUGACAUCUGAGGGA | Chmp, Rat | [1190-1208] |
| 535 | ACCAUCAUUUCGGUCAUAA | UUAUGACCGAAAUGAUGGU | Chmp | [1167-1185] |
| 536 | UCCGGAGCAAUAAGCCAGU | ACUGGCUUAUUGCUCCGGA | Chmp | [1147-1165] |
| 537 | ACAGGUAUAUCCGGAGCAA | UUGCUCCGGAUAUACCUGU | Chmp | [1138-1156] |
| 538 | CCACUCAGUCUUUGACCCU | AGGGUCAAAGACUGAGUGG | | [109-127] |
| 539 | GAACCUUUCCCUGAAGGAU | AUCCUUCAGGGAAAGGUUC | Chmp | [984-1002] |
| 540 | CAGAACAUUUUCAUGAACC | GGUUCAUGAAAAUGUUCUG | Chmp | [970-988] |
| 541 | UCUCAGAAUAUUUCCUUAC | GUAAGGAAAUAUUCUGAGA | Chmp | [939-957] |
| 542 | AUCCUAGAAAACUUCUCUU | AAGAGAAGUUUUCUAGGAU | Chmp | [688-706] |
| 543 | GGCUCUCCAUGAAUGUCCU | AGGACAUUCAUGGAGAGCC | Chmp | [304-322] |
| 544 | UGCCGAACACUCUUGGCUU | AAGCCAAGAGUGUUCGGCA | Chmp | [465-483] |
| 545 | CAAAUGCAAGAUUGAACAA | UUGUUCAAUCUUGCAUUUG | Chmp | [2345-2363] |
| 546 | UUGAUGUGAUGAUUCACUU | AAGUGAAUCAUCACAUCAA | Chmp | [2185-2203] |
| 547 | UCAGCGGAAUCAAUCAGCU | AGCUGAUUGAUUCCGCUGA | Chmp | [2040-2058] |
| 548 | UGAGUAACCAGAACAACUC | GAGUUGUUCUGGUUACUCA | | [1915-1933] |
| 549 | UCCAGCUGUACCUCAGUCA | UGACUGAGGUACAGCUGGA | Chmp, Rat, | [1720-1738] |
| | | | Ms | |
| 550 | GGCAAGAGAACAGACCUGA | UCAGGUCUGUUCUCUUGCC | Chmp | [1690-1708] |
| 551 | UUCCGUUGGUUUGCAGAUU | AAUCUGCAAACCAACGGAA | Chmp | [1644-1662] |
| 552 | GGAUGACUGGAAACCAUAC | GUAUGGUUUCCAGUCAUCC | Chmp, Rat, Ms | [1577-1595] |
| 553 | ACACCCUGUUGGAUGACUG | CAGUCAUCCAACAGGGUGU | | [1567-1585] |
| 554 | GUCCAUUUGAGGAAUCACU | AGUGAUUCCUCAAAUGGAC | Chmp | [1483-1501] |
| 555 | UGGUCCUUUUGGAAGUCCA | UGGACUUCCAAAAGGACCA | Chmp | [1469-1487] |
| 556 | AGACCUGGUCAGUAUAUUA | UAAUAUACUGACCAGGUCU | Chmp | [1239-1257] |
| 557 | CCGAACCAGCUCUCAGAAU | AUUCUGAGAGCUGGUUCGG | Chmp | [929-947] |
| 558 | UCAGUCUUAACCGAACCAG | CUGGUUCGGUUAAGACUGA | Chmp | [919-937] |
| 559 | GGCUGCAUCAGUCUUAACC | GGUUAAGACUGAUGCAGCC | Chmp | [912-930] |
| 560 | UCCUGCUUUUCUGGAAAAC | GUUUUCCAGAAAAGCAGGA | Chmp, Rat | [319-337] |
| 561 | UGGUGGUGGUGCUAUUCCU | AGGAAUAGCACCACCACCA | Chmp | [739-757] |
| 562 | CCUAGAAAACUUCUCUUCA | UGAAGAGAAGUUUUCUAGG | Chmp | [690-708] |
| 563 | CCGAGAUGAGGAUCCUAGA | UCUAGGAUCCUCAUCUCGG | Chmp | [677-695] |
| 564 | AUGUGAUGAUUCACUUUUC | GAAAAGUGAAUCAUCACAU | Chmp | [2188-2206] |
| 565 | AGCGGAAUCAAUCAGCUGU | ACAGCUGAUUGAUUCCGCU | Chmp | [2042-2060] |
| 566 | AGAGUCUUUCAGCUGAAAA | UUUUCAGCUGAAAGACUCU | Chmp | [1961-1979]+ |
| 567 | ACAGUUGGUGUUUUCUGUU | AACAGAAAACACCAACUGU | Chmp | [1860-1878] |
| 568 | GUCCUCGGUGGAAACUUUU | AAAAGUUUCCACCGAGGAC | Chmp, Ms | [1807-1825] |
| 569 | CAAGAGAACAGACCUGACU | AGUCAGGUCUGUUCUCUUG | Chmp | [1692-1710] |
| 570 | GGUUUGCAGAUUUACUCUG | CAGAGUAAAUCUGCAAACC | Chmp, Ms | [1651-1669] |
| 571 | UGGAUGACUGGAAACCAUA | UAUGGUUUCCAGUCAUCCA | Chmp, Rat, Ms | [1576-1594] |
| 572 | UGGAGUAACUCCAUUUGCA | UGCAAAUGGAGUUACUCCA | Chmp | [1538-1556] |
| 573 | GGAAUCACUGAACUAUGAG | CUCAUAGUUCAGUGAUUCC | Chmp | [1493-1511] |
| 574 | AGGUAUAUCCGGAGCAAUA | UAUUGCUCCGGAUAUACCU | Chmp | [1140-1158] |
| 575 | CCUUUCCCUGAAGGAUUUU | AAAAUCCUUCAGGGAAAGG | Chmp | [987-1005] |
| 576 | UCAGAAUAUUUCCUUACCA | UGGUAAGGAAAUAUUCUGA | Chmp | [941-959] |
| 577 | UGCUUUUCUGGAAAACCUU | AAGGUUUUCCAGAAAAGCA | Chmp, Rat | [322-340] |
| 578 | UGCAUCAGUCUUAACCGAA | UUCGGUUAAGACUGAUGCA | Chmp | [915-933] |
| 579 | CUGCAUCAGUCUUAACCGA | UCGGUUAAGACUGAUGCAG | Chmp | [914-932] |
| 580 | ACAUGCUGCUGACGUUGCA | UGCAACGUCAGCAGCAUGU | Chmp | [841-859] |
| 581 | ACCGAGAUGAGGAUCCUAG | CUAGGAUCCUCAUCUCGGU | Chmp | [676-694] |
| 582 | CUGUUCAUCUGGCUCUCCA | UGGAGAGCCAGAUGAACAG | | [294-312] |
| 583 | AAGGUUCCAAGCAGGAGAA | UUCUCCUGCUUGGAACCUU | Chmp | [501-519] |
| 584 | CUCAAUCUAAGGCUGAUUG | CAAUCAGCCUUAGAUUGAG | Chmp | [2294-2312] |
| 585 | UGGAUUCAAGAACUCCCUA | UAGGGAGUUCUUGAAUCCA | Chmp | [2258-2276] |
| 586 | ACUGUUAGAUGCACACUGU | ACAGUGUGCAUCUAACAGU | Chmp | [2227-2245] |
| 587 | CAGCUGUGUUAUGCCAAAG | CUUUGGCAUAACACAGCUG | Chmp | [2054-2072] |
| 588 | GCCAUGAAGCAGGACUCUA | UAGAGUCCUGCUUCAUGGC | Chmp | [1985-2003] |
| 589 | UCUUUCAGCUGAAAACUUU | AAAGUUUUCAGCUGAAAGA | Chmp | [1965-1983]+ |
| 590 | GAGUCUUUCAGCUGAAAAC | GUUUUCAGCUGAAAGACUC | Chmp | [1962-1980]+ |
| 591 | AGACUGUUUAUAGGACGUC | GACGUCCUAUAAACAGUCU | | [1791-1809] |
| 592 | CUGACUAUGUCAACAUCCA | UGGAUGUUGACAUAGUCAG | Chmp | [1705-1723] |
| 593 | UGGCAAGAGAACAGACCUG | CAGGUCUGUUCUCUUGCCA | Chmp | [1689-1707] |
| 594 | UCAUCCCUCAGAUGUCAUG | CAUGACAUCUGAGGGAUGA | Chmp, Rat | [1187-1205] |
| 595 | GAACAUUUUCAUGAACCUU | AAGGUUCAUGAAAAUGUUC | Chmp | [972-990] |
| 596 | CAGCCUCUACAUAUGCAAU | AUUGCAUAUGUAGAGGCUG | Chmp | [766-784] |
| 597 | CACAGCCUCUACAUAUGCA | UGCAUAUGUAGAGGCUGUG | Chmp | [764-782] |
| 598 | AGGAGAACCAGGAGAUUGU | ACAAUCUCCUGGUUCUCCU | Chmp | [513-531] |
| 599 | GAAGGUUCCAAGCAGGAGA | UCUCCUGCUUGGAACCUUC | Chmp | [500-518] |
| 600 | CUAAGACUUUGAAACUCAG | CUGAGUUUCAAAGUCUUAG | Chmp | [2025-2043] |
| 601 | AAGAGUCUUUCAGCUGAAA | UUUCAGCUGAAAGACUCUU | Chmp | [1960-1978]+ |
| 602 | GAAAAACAGUUGGUGUUUU | AAAACACCAACUGUUUUUC | | [1855-1873] |
| 603 | GCAAGAGAACAGACCUGAC | GUCAGGUCUGUUCUCUUGC | Chmp | [1691-1709] |
| 604 | UGAACUAUGAGGUCAGCCU | AGGCUGACCUCAUAGUUCA | Chmp | [1501-1519] |
| 605 | CACUGAACUAUGAGGUCAG | CUGACCUCAUAGUUCAGUG | Chmp | [1498-1516] |
| 606 | UCACUGAACUAUGAGGUCA | UGACCUCAUAGUUCAGUGA | Chmp | [1497-1515] |
| 607 | UGGAAAACCUUCUUGCUGU | ACAGCAAGAAGGUUUUCCA | Chmp | [330-348] |
| 608 | CAGCAAGAUACCGAGAUGA | UCAUCUCGGUAUCUUGCUG | Chmp | [667-685] |
| 609 | UCAAUCUAAGGCUGAUUGU | ACAAUCAGCCUUAGAUUGA | Chmp | [2295-2313] |
| 610 | AGAACUCCCUAGUGAGGAG | CUCCUCACUAGGGAGUUCU | Chmp | [2266-2284] |
| 611 | CACUUUUCAGUUCUCAAAU | AUUUGAGAACUGAAAAGUG | Chmp | [2199-2217] |
| 612 | UGAUGAUUCACUUUUCAGU | ACUGAAAAGUGAAUCAUCA | Chmp | [2191-2209] |
| 613 | AGCUGAAAACUUUUGCCAU | AUGGCAAAAGUUUUCAGCU | Chmp | [1971-1989]+ |
| 614 | CAGCUGAAAACUUUUGCCA | UGGCAAAAGUUUUCAGCUG | Chmp | [1970-1988]+ |
| 615 | AGACUCUUCAUAAACUGAG | CUCAGUUUAUGAAGAGUCU | Chmp | [1900-1918] |
| 616 | CUGUACCUCAGUCAAACAG | CUGUUUGACUGAGGUACAG | Chmp, Rat, Ms | [1725-1743] |
| 617 | ACAUCCAGCUGUACCUCAG | CUGAGGUACAGCUGGAUGU | Chmp, Rat, Ms | [1717-1735] |
| 618 | UCAACAUCCAGCUGUACCU | AGGUACAGCUGGAUGUUGA | Chmp | [1714-1732] |
| 619 | AUGACUGGAAACCAUACAA | UUGUAUGGUUUCCAGUCAU | Chmp, Rat, Ms | [1579-1597] |
| 620 | AGGCAUUGGAGUAACUCCA | UGGAGUUACUCCAAUGCCU | Chmp | [1532-1550] |
| 621 | ACCCAGCACAAAUUUGUGA | UCACAAAUUUGUGCUGGGU | Chmp | [1023-1041] |
| 622 | ACCGAACCAGCUCUCAGAA | UUCUGAGAGCUGGUUCGGU | Chmp | [928-946] |
| 623 | UGUCCUGCUUUUCUGGAAA | UUUCCAGAAAAGCAGGACA | Chmp, Rat | [317-335] |
| 624 | CUGUUAGAUGCACACUGUU | AACAGUGUGCAUCUAACAG | Chmp, Rat | [2228-2246] |
| 625 | GAAAACUGUUAGAUGCACA | UGUGCAUCUAACAGUUUUC | Chmp | [2223-2241] |
| 626 | UGUGAUGAUUCACUUUUCA | UGAAAAGUGAAUCAUCACA | Chmp | [2189-2207] |
| 627 | ACUCUUCAUAAACUGAGUA | UACUCAGUUUAUGAAGAGU | Chmp | [1902-1920] |
| 628 | GACUCUUCAUAAACUGAGU | ACUCAGUUUAUGAAGAGUC | Chmp | [1901-1919] |
| 629 | CAGAGCCUCAGCAUCUGUU | AACAGAUGCUGAGGCUCUG | Chmp | [410-428] |
| 630 | CUAAGCAGAGCCUCAGCAU | AUGCUGAGGCUCUGCUUAG | Chmp | [405-423] |
| 631 | UUGGAUGACUGGAAACCAU | AUGGUUUCCAGUCAUCCAA | Chmp | [1575-1593] |
| 632 | ACAUUUUCAUGAACCUUUC | GAAAGGUUCAUGAAAAUGU | Chmp | [974-992] |
| 633 | AGAACAUUUUCAUGAACCU | AGGUUCAUGAAAAUGUUCU | Chmp | [971-989] |
| 634 | GAUUCACUUUUCAGUUCUC | GAGAACUGAAAAGUGAAUC | Chmp | [2195-2213] |
| 635 | UGGAAAUCCGAAUGGUCAA | UUGACCAUUCGGAUUUCCA | Chmp | [1204-1222] |
| 636 | UCCCUGAAGGAUUUUCAAA | UUUGAAAAUCCUUCAGGGA | Chmp | [991-1009] |
| 637 | GGAUCACAGAAGGUUCCAA | UUGGAACCUUCUGUGAUCC | Chmp | [492-510] |
| 638 | CAGAAUAUUUCCUUACCAG | CUGGUAAGGAAAUAUUCUG | Chmp | [942-960] |
| 639 | AAAAACAGUUGGUGUUUUC | GAAAACACCAACUGUUUUU | | [1856-1874] |
| 640 | GGCUUUGGAUUUCUGGACC | GGUCCAGAAAUCCAAAGCC | Chmp | [1090-1108] |
| 641 | GUCUUCUACAUGCUGCUGA | UCAGCAGCAUGUAGAAGAC | Chmp | [834-852] |
| 642 | GUUGAACUGAAUGCAGCAA | UUGCUGCAUUCAGUUCAAC | Chmp | [654-672] |
| 643 | AUGUCCUGCUUUUCUGGAA | UUCCAGAAAAGCAGGACAU | Chmp, Rat | [316-334] |
| 644 | UGCCUGUUCAUCUGGCUCU | AGAGCCAGAUGAACAGGCA | | [291-309] |

TABLE A2
NOX4 - NADPH oxidase 4 (19-mers)

| | | | | Human-20149638 |
|---|---|---|---|---|
| No. | Sense siRNA | AntiSense siRNA | Other Sp | ORF: 240-1976 |
| 1 | GGACAAGAUUUGAAUACAA | UUGUAUUCAAAUCUUGUCC | | [1939-1957] |
| 2 | GAAGAUAAUUGGAGAAAAA | UUUUUCUCCAAUUAUCUUC | Rat, Ms, GP, Chmp | [1754-1772] |
| 3 | CCAGGAGAUUGUUGGAUAA | UUAUCCAACAAUCUCCUGG | Chmp | [520-538] |
| 4 | GAAGCAGGACUCUAAAGAA | UUCUUUAGAGUCCUGCUUC | Chmp | [1990-2008] |
| 5 | CAUGAAGCAGGACUCUAAA | UUUAGAGUCCUGCUUCAUG | Chmp | [1987-2005] |
| 6 | CAGUGUAUCUGCAUUAGAA | UUCUAAUGCAGAUACACUG | Chmp | [1271-1289] |
| 7 | GCUGAAGUAUCAAACUAAU | AUUAGUUUGAUACUUCAGC | Chmp | [875-893] |
| 8 | GGUUCAUCUUAAAAUAGUA | UACUAUUUUAAGAUGAACC | Chmp | [1343-1361] |
| 9 | CCAUGAAGCAGGACUCUAA | UUAGAGUCCUGCUUCAUGG | Chmp | [1986-2004] |
| 10 | GGUCAAAGAAAAUUUUAAA | UUUAAAAUUUUCUUUGACC | GP, Chmp | [1217-1235] |
| 11 | CCAAAGAGACCCUGAAGAA | UUCUUCAGGGUCUCUUUGG | Chmp | [2163-2181] |
| 12 | GUUUAAUCUGGAAACCAAA | UUUGGUUUCCAGAUUAAAC | Chmp | [2149-2167] |
| 13 | GAAUGAGUGCAAUUUCUAA | UUAGAAAUUGCACUCAUUC | Chmp | [2010-2028] |
| 14 | GAUGAAAUAGCAAAAUAUA | UAUAUUUUGCUAUUUCAUC | Chmp | [1830-1848] |
| 15 | AAUGCAAGAUUGAACAAAA | UUUUGUUCAAUCUUGCAUU | Chmp | [2347-2365] |
| 16 | CGUCCUCGGUGGAAACUUU | AAAGUUUCCACCGAGGACG | Ms, Chmp | [1806-1824] |
| 17 | AAACCUUCUUGCUGUAUAA | UUAUACAGCAAGAAGGUUU | Chmp | [334-352] |
| 18 | GAAACUUUUGUUUGAUGAA | UUCAUCAAACAAAAGUUUC | Chmp | [1817-1835] |
| 19 | GCAGGACUCUAAAGAAGGA | UCCUUCUUUAGAGUCCUGC | Chmp | [1993-2011] |
| 20 | CAAGAUUUGAAUACAAUAA | UUAUUGUAUUCAAAUCUUG | Chmp | [1942-1960] |
| 21 | CAAUUUCUAAGACUUUGAA | UUCAAAGUCUUAGAAAUUG | Chmp | [2019-2037] |
| 22 | GAGGGCUGCUGAAGUAUCA | UGAUACUUCAGCAGCCCUC | Chin, GP, Chmp | [868-886] |
| 23 | GAACAAAUGCAAGAUUGAA | UUCAAUCUUGCAUUUGUUC | Chmp | [2342-2360] |
| 24 | ACAAGAUUUGAAUACAAUA | UAUUGUAUUCAAAUCUUGU | Chmp | [1941-1959] |
| 25 | AGAUAAUUGGAGAAAAAUA | UAUUUUUCUCCAAUUAUCU | Rat, Ms, GP, Chmp | [1756-1774] |
| 26 | GACCCUGAAGAAUAUUUGA | UCAAAUAUUCUUCAGGGUC | Chmp | [2170-2188] |
| 27 | GCAAGAUUGAACAAAAUUA | UAAUUUUGUUCAAUCUUGC | Chmp | [2350-2368] |
| 28 | GAUGGGAUACAGAAGAUAA | UUAUCUUCUGUAUCCCAUC | Rat, GP, Chmp | [1743-1761] |
| 29 | UGUCGUGUUCCUCUUUAAA | UUUAAAGAGGAACACGACA | Chmp | [2311-2329] |
| 30 | GGGACAAGAUUUGAAUACA | UGUAUUCAAAUCUUGUCCC | | [1938-1956] |
| 31 | UGGUCAAAGAAAAUUUUAA | UUAAAAUUUUCUUUGACCA | GP, Chmp | [1216-1234] |
| 32 | CAUUCAAUCUAGAAAUUAU | AUAAUUUCUAGAUUGAAUG | Chin, Chmp | [1433-1451] |
| 33 | AAGAGACCCUGAAGAAUAU | AUAUUCUUCAGGGUCUCUU | Chmp | [2166-2184] |
| 34 | GAAGUAUCAAACUAAUUUA | UAAAUUAGUUUGAUACUUC | Chmp | [878-896] |
| 35 | GUUUGAUGAAAUAGCAAAA | UUUUGCUAUUUCAUCAAAC | GP, Chmp | [1826-1844] |
| 36 | GAACCAGGAGAUUGUUGGA | UCCAACAAUCUCCUGGUUC | Chmp | [517-535] |
| 37 | GACAAGAUUUGAAUACAAU | AUUGUAUUCAAAUCUUGUC | Chmp | [1940-1958] |
| 38 | GUACCUCAGUCAAACAGAU | AUCUGUUUGACUGAGGUAC | Rat, Ms, Chmp | [1727-1745] |
| 39 | UUUCAGUUCUCAAAUUAAA | UUUAAUUUGAGAACUGAAA | Chmp | [2203-2221] |
| 40 | CAAAGAAAAUUUUAAAGCA | UGCUUUAAAAUUUUCUUUG | GP, Chmp | [1220-1238] |
| 41 | GCAUGGUGGUGGUGCUAUU | AAUAGCACCACCACCAUGC | Chmp | [736-754] |
| 42 | GAAACUCAGCGGAAUCAAU | AUUGAUUCCGCUGAGUUUC | Chmp | [2035-2053] |
| 43 | GGAAAACCUUCUUGCUGUA | UACAGCAAGAAGGUUUUCC | Chmp | [331-349] |
| 44 | AGAAGAUAAUUGGAGAAAA | UUUUCUCCAAUUAUCUUCU | Rat, Ms, GP, Chmp | [1753-1771] |
| 45 | CAAGAUACCGAGAUGAGGA | UCCUCAUCUCGGUAUCUUG | GP, Chmp | [670-688] |
| 46 | AGAAAAUUUUAAAGCAAGA | UCUUGCUUUAAAAUUUUCU | GP, Chmp | [1223-1241] |
| 47 | AGGGCUGCUGAAGUAUCAA | UUGAUACUUCAGCAGCCCU | Chin, GP, Chmp | [869-887] |
| 48 | CAAACAGAUGGGAUACAGA | UCUGUAUCCCAUCUGUUUG | Rat, GP, Chmp | [1737-1755] |
| 49 | AUGCAAGAUUGAACAAAAU | AUUUUGUUCAAUCUUGCAU | Chmp | [2348-2366] |
| 50 | GAUAAUUGGAGAAAAAUAU | AUAUUUUUCUCCAAUUAUC | Ms, GP, Chmp | [1757-1775] |
| 51 | CUUAGAAGACUAUACUUUA | UAAAGUAUAGUCUUCUAAG | Chmp | [1599-1617] |
| 52 | AAACCAUACAAGCUUAGAA | UUCUAAGCUUGUAUGGUUU | Chin, GP, Chmp | [1587-1605] |
| 53 | AGUGUAUCUGCAUUAGAAA | UUUCUAAUGCAGAUACACU | Chmp | [1272-1290] |
| 54 | AGCAGAACAUUCCAUAUUA | UAAUAUGGAAUGUUCUGCU | Chmp | [540-558] |
| 55 | CCAUUUGCAUCAAUACUCA | UGAGUAUUGAUGCAAAUGG | Chmp | [1548-1566] |
| 56 | ACUUUUGUUUGAUGAAAUA | UAUUUCAUCAAACAAAAGU | Chmp | [1820-1838] |
| 57 | CGAACCAGCUCUCAGAAUA | UAUUCUGAGAGCUGGUUCG | Chmp | [930-948] |
| 58 | CUUCUUGCUGUAUAACCAA | UUGGUUAUACAGCAAGAAG | Chmp | [338-356] |
| 59 | UUCAGUUCUCAAAUUAAAA | UUUUAAUUUGAGAACUGAA | Chmp | [2204-2222] |
| 60 | GUAUCAAACUAAUUUAGAU | AUCUAAAUUAGUUUGAUAC | Chmp | [881-899] |
| 61 | CAAUAAGAGUUUCUAACUA | UAGUUAGAAACUCUUAUUG | Chmp | [781-799] |
| 62 | GCUGUACCUCAGUCAAACA | UGUUUGACUGAGGUACAGC | Rat, Ms, Chmp | [1724-1742] |
| 63 | AUACAAGCUUAGAAGACUA | UAGUCUUCUAAGCUUGUAU | Chmp | [1592-1610] |
| 64 | UCAUGAACCUUUCCCUGAA | UUCAGGGAAAGGUUCAUGA | Chmp | [980-998] |
| 65 | UGUUGAUUUUCAUGGUGGA | UCCACCAUGAAAAUCAACA | | [2243-2261] |
| 66 | CAUUUGCAUCAAUACUCAA | UUGAGUAUUGAUGCAAAUG | Chmp | [1549-1567] |
| 67 | GGAAUGAGUGCAAUUUCUA | UAGAAAUUGCACUCAUUCC | Chmp | [2009-2027] |
| 68 | UGAUGAAAUAGCAAAAUAU | AUAUUUUGCUAUUUCAUCA | Chmp | [1829-1847] |
| 69 | AAACAGAUGGGAUACAGAA | UUCUGUAUCCCAUCUGUUU | Rat, GP, Chmp | [1738-1756] |
| 70 | GAAUUACAGUGAAGACUUU | AAAGUCUUCACUGUAAUUC | | [635-653] |
| 71 | UGGAAACCAUACAAGCUUA | UAAGCUUGUAUGGUUUCCA | Chin, GP, Chmp | [1584-1602] |
| 72 | GUUCUCAAAUUAAAAGAAA | UUUCUUUUAAUUUGAGAAC | Chmp | [2208-2226] |
| 73 | GAAUAGUAAGGUUUUCUUA | UAAGAAAACCUUACUAUUC | Chmp | [2072-2090] |
| 74 | AACUGAAACCAAAGCAACA | UGUUGCUUUGGUUUCAGUU | Chmp | [1319-1337] |
| 75 | UUUGGUUGAACAAAUGCAA | UUGCAUUUGUUCAACCAAA | Chmp | [2335-2353] |
| 76 | AAUUCUGCCCUUCAUUCAA | UUGAAUGAAGGGCAGAAUU | Chmp | [1421-1439] |
| 77 | AUGCAAUAAGAGUUUCUAA | UUAGAAACUCUUAUUGCAU | Rat, Chmp | [778-796] |
| 78 | UUCCCUGAAGGAUUUUCAA | UUGAAAAUCCUUCAGGGAA | Chmp | [990-1008] |
| 79 | CUGCUGAAGUAUCAAACUA | UAGUUUGAUACUUCAGCAG | Chmp | [873-891] |
| 80 | GAAGAAUAUUUGAUGUGAU | AUCACAUCAAAUAUUCUUC | Chmp | [2176-2194] |
| 81 | GAGCCCAGAUUCCAAGCUA | UAGCUUGGAAUCUGGGCUC | Chmp | [1056-1074] |
| 82 | AGAUGUCAUGGAAAUCCGA | UCGGAUUUCCAUGACAUCU | GP, Chin, Chmp | [1196-1214] |
| 83 | AAGAGUUUCUAACUAUGAU | AUCAUAGUUAGAAACUCUU | Chmp | [785-803] |
| 84 | GAGUAACCAGAACAACUCA | UGAGUUGUUCUGGUUACUC | | [1916-1934] |
| 85 | AGAACGAUUUCGAGAUUUA | UAAAUCUCGAAAUCGUUCU | Chmp | [1373-1391] |
| 86 | CAAAGAGACCCUGAAGAAU | AUUCUUCAGGGUCUCUUUG | Chmp | [2164-2182] |
| 87 | GAUGCACACUGUUGAUUUU | AAAAUCAACAGUGUGCAUC | Rat, Chmp | [2234-2252] |
| 88 | UAUCCAAGACUCUUCAUAA | UUAUGAAGAGUCUUGGAUA | Chmp | [1894-1912] |
| 89 | GUAAGGUUUUCUUAUUUAU | AUAAAUAAGAAAACCUUAC | Chmp | [2077-2095] |
| 90 | AUCCGAAUGGUCAAAGAAA | UUUCUUUGACCAUUCGGAU | GP, Chin, Chmp | [1209-1227] |
| 91 | UAUCAUGCACUGAAUUCAA | UUGAAUUCAGUGCAUGAUA | Chmp | [1773-1791] |
| 92 | GAUACAGAAGAUAAUUGGA | UCCAAUUAUCUUCUGUAUC | Rat, GP, Chmp | [1748-1766] |
| 93 | CCUCAGAUGUCAUGGAAAU | AUUUCCAUGACAUCUGAGG | Chmp | [1192-1210] |
| 94 | UAUUGAUGGUCCUUUUGGA | UCCAAAAGGACCAUCAAUA | Chmp | [1463-1481] |
| 95 | GUUGGAUAAAAGCAGAACA | UGUUCUGCUUUUAUCCAAC | Chmp | [530-548] |
| 96 | GAUGUCAUGGAAAUCCGAA | UUCGGAUUUCCAUGACAUC | GP, Chin, Chmp | [1197-1215] |
| 97 | GAGAUGAGGAUCCUAGAAA | UUUCUAGGAUCCUCAUCUC | GP, Chmp | [679-697] |
| 98 | UGAACUGAAUGCAGCAAGA | UCUUGCUGCAUUCAGUUCA | Ms, GP, Chmp | [656-674] |
| 99 | AGGAAUCACUGAACUAUGA | UCAUAGUUCAGUGAUUCCU | Chmp | [1492-1510] |
| 100 | GAAAUUAUCCCAAGCUGUA | UACAGCUUGGGAUAAUUUC | Chmp | [1444-1462] |
| 101 | AGGUUUUCUUAUUUAUGAU | AUCAUAAAUAAGAAAACCU | Chmp | [2080-2098] |
| 102 | AAAGAGACCCUGAAGAAUA | UAUUCUUCAGGGUCUCUUU | Chmp | [2165-2183] |
| 103 | GAUUUGAAUACAAUAAAGA | UCUUUAUUGUAUUCAAAUC | Chmp | [1945-1963] |
| 104 | CAUGGUGGAUUCAAGAACU | AGUUCUUGAAUCCACCAUG | | [2253-2271] |
| 105 | GUUCUUAACCUCAACUGCA | UGCAGUUGAGGUUAAGAAC | Chmp | [426-444] |
| 106 | AAAAUAUAACAGAGGAAAA | UUUUCCUCUGUUAUAUUUU | | [1841-1859] |
| 107 | CAGAUGGGAUACAGAAGAU | AUCUUCUGUAUCCCAUCUG | Rat, GP, Chmp | [1741-1759] |
| 108 | AAGGUUUUCUUAUUUAUGA | UCAUAAAUAAGAAAACCUU | Chmp | [2079-2097] |
| 109 | AGAAUAUUUCCUUACCAGA | UCUGGUAAGGAAAUAUUCU | Chmp | [943-961] |
| 110 | CAUCAAUACUCAACACCCU | AGGGUGUUGAGUAUUGAUG | | [1555-1573] |
| 111 | UCUAGUCAAGACUCCGAAA | UUUCGGAGUCUUGACUAGA | Chmp | [1404-1422] |
| 112 | GAUGACUGGAAACCAUACA | UGUAUGGUUUCCAGUCAUC | Rat, Ms, Chin, GP, Chmp | [1578-1596] |
| 113 | CAUGAUCACAGCCUCUACA | UGUAGAGGCUGUGAUCAUG | Chmp | [758-776] |
| 114 | GUAUAUUACUCUACAUUGU | ACAAUGUAGAGUAAUAUAC | Chmp | [1250-1268] |
| 115 | UUACAUGUUUAAUCUGGAA | UUCCAGAUUAAACAUGUAA | Chmp | [2143-2161] |
| 116 | UUGUGGACCCAAUUCACUA | UAGUGAAUUGGGUCCACAA | Chmp | [1877-1895] |
| 117 | AACUCAUAUGGGACAAGAU | AUCUUGUCCCAUAUGAGUU | | [1929-1947] |
| 118 | AAUUUCUAAGACUUUGAAA | UUUCAAAGUCUUAGAAAUU | Chmp | [2020-2038] |
| 119 | AAUCCGAAUGGUCAAAGAA | UUCUUUGACCAUUCGGAUU | GP, Chin, Chmp | [1208-1226] |
| 120 | CAUCAUUUCGGUCAUAAGU | ACUUAUGACCGAAAUGAUG | Chmp | [1169-1187] |
| 121 | GAACCAGCUCUCAGAAUAU | AUAUUCUGAGAGCUGGUUC | Chmp | [931-949] |
| 122 | AGUUCUCAAAUUAAAAGAA | UUCUUUUAAUUUGAGAACU | Chmp | [2207-2225] |
| 123 | GAAAUAGCAAAAUAUAACA | UGUUAUAUUUUGCUAUUUC | Chmp | [1833-1851] |
| 124 | GACUCUAAAGAAGGAAUGA | UCAUUCCUUCUUUAGAGUC | Chmp | [1997-2015] |
| 125 | CAAGCUUAGAAGACUAUAC | GUAUAGUCUUCUAAGCUUG | Chmp | [1595-1613] |
| 126 | AAAAUAUCAUGCACUGAAU | AUUCAGUGCAUGAUAUUUU | Chmp | [1769-1787] |
| 127 | GUGCUAUUCCUCAUGAUCA | UGAUCAUGAGGAAUAGCAC | Chmp | [747-765] |
| 128 | GUACUGUGCCGAAAGACUU | AAGUCUUUCGGCACAGUAC | Chin, Chmp | [1118-1136] |
| 129 | CAACUCAUAUGGGACAAGA | UCUUGUCCCAUAUGAGUUG | | [1928-1946] |
| 130 | AGAUUUGAAUACAAUAAAG | CUUUAUUGUAUUCAAAUCU | Chmp | [1944-1962] |
| 131 | AAUUGUUUUUGGUUGAACA | UGUUCAACCAAAAACAAUU | Chmp | [2328-2346] |
| 132 | UGUUUUUGGUUGAACAAAU | AUUUGUUCAACCAAAAACA | Chmp | [2331-2349] |
| 133 | AACUGAAUGCAGCAAGAUA | UAUCUUGCUGCAUUCAGUU | Ms, GP, Chmp | [658-676] |
| 134 | UGUGCCGAAAGACUUUACA | UGUAAAGUCUUUCGGCACA | Chin, Chmp | [1122-1140] |
| 135 | AACCGAACCAGCUCUCAGA | UCUGAGAGCUGGUUCGGUU | Chmp | [927-945] |
| 136 | AUGGGACAAGAUUUGAAUA | UAUUCAAAUCUUGUCCCAU | | [1936-1954] |
| 137 | GUUAGAUGCACACUGUUGA | UCAACAGUGUGCAUCUAAC | Rat, Chmp | [2230-2248] |
| 138 | GCAGAGAUAUCCAGUCCUU | AAGGACUGGAUAUCUCUGC | Chmp | [1627-1645] |
| 139 | UCAGUAUAUUACUCUACAU | AUGUAGAGUAAUAUACUGA | Chmp | [1247-1265] |
| 140 | AAAUCCGAAUGGUCAAAGA | UCUUUGACCAUUCGGAUUU | GP, Chin, Chmp | [1207-1225] |
| 141 | GUUGGUUUGCAGAUUUACU | AGUAAAUCUGCAAACCAAC | Chmp | [1648-1666] |
| 142 | CAUAAACUGAGUAACCAGA | UCUGGUUACUCAGUUUAUG | Chmp | [1908-1926] |
| 143 | CACCAUCAUUUCGGUCAUA | UAUGACCGAAAUGAUGGUG | Chmp | [1166-1184] |
| 144 | UACAGAAGAUAAUUGGAGA | UCUCCAAUUAUCUUCUGUA | Rat, GP, Chmp | [1750-1768] |
| 145 | AAACUUUUGUUUGAUGAAA | UUUCAUCAAACAAAAGUUU | Chmp | [1818-1836] |
| 146 | ACCUCCGAGGAUCACAGAA | UUCUGUGAUCCUCGGAGGU | Chmp | [484-502] |
| 147 | ACAAGCUUAGAAGACUAUA | UAUAGUCUUCUAAGCUUGU | Chmp | [1594-1612] |
| 148 | CCUUCCGUUGGUUUGCAGA | UCUGCAAACCAACGGAAGG | Chmp | [1642-1660] |
| 149 | GCAGAACAUUCCAUAUUAC | GUAAUAUGGAAUGUUCUGC | Chmp | [541-559] |
| 150 | GAGACUGGACAGAACGAUU | AAUCGUUCUGUCCAGUCUC | Ms, Chin, GP, Chmp | [1363-1381] |
| 151 | UACUCAACACCCUGUUGGA | UCCAACAGGGUGUUGAGUA | | [1561-1579] |
| 152 | UAGAAAACUUCUCUUCACA | UGUGAAGAGAAGUUUUCUA | GP, Chmp | [692-710] |
| 153 | AGGAGACUGGACAGAACGA | UCGUUCUGUCCAGUCUCCU | Ms, Chin, GP, Chmp | [1361-1379] |
| 154 | UUUUCAGUUCUCAAAUUAA | UUAAUUUGAGAACUGAAAA | Chmp | [2202-2220] |
| 155 | AGAGUUUCUAACUAUGAUA | UAUCAUAGUUAGAAACUCU | Chmp | [786-804] |
| 156 | UCAUUUCGGUCAUAAGUCA | UGACUUAUGACCGAAAUGA | Chmp | [1171-1189] |
| 157 | GCUGACGUUGCAUGUUUCA | UGAAACAUGCAACGUCAGC | Chmp | [848-866] |
| 158 | CGUUGGUUUGCAGAUUUAC | GUAAAUCUGCAAACCAACG | Chmp | [1647-1665] |
| 159 | CAGUUCUCAAAUUAAAAGA | UCUUUUAAUUUGAGAACUG | Chmp | [2206-2224] |
| 160 | UUUCAGGAGGGCUGCUGAA | UUCAGCAGCCCUCCUGAAA | GP, Chmp | [862-880] |
| 161 | UGUUGUGGACCCAAUUCAC | GUGAAUUGGGUCCACAACA | Chmp | [1875-1893] |
| 162 | GAAACCAUACAAGCUUAGA | UCUAAGCUUGUAUGGUUUC | Chin, GP, Chmp | [1586-1604] |
| 163 | AUUUGGGUAUGCAGAGAUA | UAUCUCUGCAUACCCAAAU | Chmp | [1617-1635] |
| 164 | AUCCAUUUACCCUCACAAU | AUUGUGAGGGUAAAUGGAU | GP, Chmp | [1294-1312] |
| 165 | AAAUAUAACAGAGGAAAAA | UUUUUCCUCUGUUAUAUUU | | [1842-1860] |
| 166 | AGUAGGAGACUGGACAGAA | UUCUGUCCAGUCUCCUACU | Rat, Ms, Chin, GP, Chmp | [1358-1376] |
| 167 | GAAACCAAAGAGACCCUGA | UCAGGGUCUCUUUGGUUUC | Chmp | [2159-2177] |
| 168 | GAAUAUUUGAUGUGAUGAU | AUCAUCACAUCAAAUAUUC | Chmp | [2179-2197] |
| 169 | AAGCAAGACCUGGUCAGUA | UACUGACCAGGUCUUGCUU | GP, Chmp | [1234-1252] |
| 170 | CUUCAUUCAAUCUAGAAAU | AUUUCUAGAUUGAAUGAAG | Chin, Chmp | [1430-1448] |
| 171 | AUCCCAAGCUGUAUAUUGA | UCAAUAUACAGCUUGGGAU | Chmp | [1450-1468] |
| 172 | ACCAGGAGAUUGUUGGAUA | UAUCCAACAAUCUCCUGGU | Chmp | [519-537] |
| 173 | GAACGAUUUCGAGAUUUAC | GUAAAUCUCGAAAUCGUUC | Chmp | [1374-1392] |
| 174 | AAACUAAUUUAGAUACCCA | UGGGUAUCUAAAUUAGUUU | Chmp | [886-904] |
| 175 | CAAAGAAUAGUAAGGUUUU | AAAACCUUACUAUUCUUUG | Chmp | [2068-2086] |
| 176 | GUAGGAGACUGGACAGAAC | GUUCUGUCCAGUCUCCUAC | Ms, Chin, GP, Chmp | [1359-1377] |
| 177 | AGUAAGGUUUUCUUAUUUA | UAAAUAAGAAAACCUUACU | Chmp | [2076-2094] |
| 178 | GAUCACAGCCUCUACAUAU | AUAUGUAGAGGCUGUGAUC | Chmp | [761-779] |
| 179 | UGGUGUUUUCUGUUGUGGA | UCCACAACAGAAAACACCA | Chmp | [1865-1883] |
| 180 | AACCUCAACUGCAGCCUUA | UAAGGCUGCAGUUGAGGUU | Chin, Chmp | [432-450] |
| 181 | CUGAGUAACCAGAACAACU | AGUUGUUCUGGUUACUCAG | | [1914-1932] |
| 182 | UUUGUUUGAUGAAAUAGCA | UGCUAUUUCAUCAAACAAA | Chmp | [1823-1841] |
| 183 | CAGAAGGUUCCAAGCAGGA | UCCUGCUUGGAACCUUCUG | Chin, GP, Chmp | [498-516] |
| 184 | UAUGGGACAAGAUUUGAAU | AUUCAAAUCUUGUCCCAUA | | [1935-1953] |
| 185 | CAUCUAGUCAAGACUCCGA | UCGGAGUCUUGACUAGAUG | Chmp | [1402-1420] |
| 186 | CUGAAGUAUCAAACUAAUU | AAUUAGUUUGAUACUUCAG | Chmp | [876-894] |
| 187 | UAUGAUAUCUUCUGGUAUA | UAUACCAGAAGAUAUCAUA | Rat, Ms, Chin, GP, Chmp | [798-816] |
| 188 | GAGGAAAAACAGUUGGUGU | ACACCAACUGUUUUUCCUC | | [1852-1870] |
| 189 | AACUUUUGUUUGAUGAAAU | AUUUCAUCAAACAAAAGUU | Chmp | [1819-1837] |
| 190 | CAUACAAGCUUAGAAGACU | AGUCUUCUAAGCUUGUAUG | Chin, GP, Chmp | [1591-1609] |
| 191 | GUUCCAAGCAGGAGAACCA | UGGUUCUCCUGCUUGGAAC | Chmp | [504-522] |
| 192 | GUGUCUAAGCAGAGCCUCA | UGAGGCUCUGCUUAGACAC | Chmp | [401-419] |
| 193 | UACUGUGCCGAAAGACUUU | AAAGUCUUUCGGCACAGUA | Chin, Chmp | [1119-1137] |
| 194 | UUAGAAGACUAUACUUUAU | AUAAAGUAUAGUCUUCUAA | Chmp | [1600-1618] |
| 195 | GGAAGUCCAUUUGAGGAAU | AUUCCUCAAAUGGACUUCC | Chin, Chmp | [1479-1497] |
| 196 | CAUCCAGCUGUACCUCAGU | ACUGAGGUACAGCUGGAUG | Rat, Ms, Chmp | [1718-1736] |
| 197 | CUACAUAUGCAAUAAGAGU | ACUCUUAUUGCAUAUGUAG | Chmp | [772-790] |
| 198 | AACUCAGCGGAAUCAAUCA | UGAUUGAUUCCGCUGAGUU | Chmp | [2037-2055] |
| 199 | AGAAGACUAUACUUUAUUU | AAAUAAAGUAUAGUCUUCU | Chmp | [1602-1620] |
| 200 | UGAAUUCAAGACUGUUUAU | AUAAACAGUCUUGAAUUCA | Chmp | [1783-1801] |
| 201 | AAGAGAACAGACCUGACUA | UAGUCAGGUCUGUUCUCUU | Chin, GP, Chmp | [1693-1711] |
| 202 | AAAGCAGAACAUUCCAUAU | AUAUGGAAUGUUCUGCUUU | Chmp | [538-556] |
| 203 | ACCAUACAAGCUUAGAAGA | UCUUCUAAGCUUGUAUGGU | Chin, GP, Chmp | [1589-1607] |
| 204 | GUGGUGCUAUUCCUCAUGA | UCAUGAGGAAUAGCACCAC | Chmp | [744-762] |
| 205 | GCCCUCAACUUCUCAGUGA | UCACUGAGAAGUUGAGGGC | Chmp | [618-636] |
| 206 | UUGUUUGAUGAAAUAGCAA | UUGCUAUUUCAUCAAACAA | Chmp | [1824-1842] |
| 207 | UUGAUGAAAUAGCAAAAUA | UAUUUUGCUAUUUCAUCAA | Chmp | [1828-1846] |
| 208 | UAACCAGAACAACUCAUAU | AUAUGAGUUGUUCUGGUUA | | [1919-1937] |
| 209 | AUUGUUGGAUAAAAGCAGA | UCUGCUUUUAUCCAACAAU | Chmp | [527-545] |
| 210 | UGUUUGAUGAAAUAGCAAA | UUUGCUAUUUCAUCAAACA | GP, Chmp | [1825-1843] |
| 211 | UGUACCUCAGUCAAACAGA | UCUGUUUGACUGAGGUACA | Rat, Ms, Chmp | [1726-1744] |
| 212 | UACAAUAAAGAGUCUUUCA | UGAAAGACUCUUUAUUGUA | Chmp | [1953-1971] |
| 213 | AAUUGGAGAAAAAUAUCAU | AUGAUAUUUUUCUCCAAUU | GP, Chmp | [1760-1778] |
| 214 | CUGUAUGUUGCAUAACAAG | CUUGUUAUGCAACAUACAG | Chmp | [1667-1685] |
| 215 | GUAACCAGAACAACUCAUA | UAUGAGUUGUUCUGGUUAC | | [1918-1936] |
| 216 | UUUCAUGGUGGAUUCAAGA | UCUUGAAUCCACCAUGAAA | | [2250-2268] |
| 217 | CAAAAUAUAACAGAGGAAA | UUUCCUCUGUUAUAUUUUG | | [1840-1858] |
| 218 | AAAAAUAUCAUGCACUGAA | UUCAGUGCAUGAUAUUUUU | Chmp | [1768-1786] |
| 219 | GACUGGAAACCAUACAAGC | GCUUGUAUGGUUUCCAGUC | Rat, Chin, GP, Chmp | [1581-1599] |
| 220 | CAAAUUAAAAGAAAACUGU | ACAGUUUUCUUUUAAUUUG | Chmp | [2213-2231] |
| 221 | AUCUAGUCAAGACUCCGAA | UUCGGAGUCUUGACUAGAU | Chmp | [1403-1421] |
| 222 | AGUGAAUUACAGUGAAGAC | GUCUUCACUGUAAUUCACU | | [632-650] |
| 223 | AUUCCAAGCUAAUUUUCCA | UGGAAAAUUAGCUUGGAAU | Chmp | [1064-1082] |
| 224 | CCAGAUUCCAAGCUAAUUU | AAAUUAGCUUGGAAUCUGG | Chmp | [1060-1078] |
| 225 | CUUUAUUUGGGUAUGCAGA | UCUGCAUACCCAAAUAAAG | Chmp | [1613-1631] |
| 226 | AUUGUUUUUGGUUGAACAA | UUGUUCAACCAAAAACAAU | Chmp | [2329-2347] |
| 227 | GUUGGAUGACUGGAAACCA | UGGUUUCCAGUCAUCCAAC | Chmp | [1574-1592] |
| 228 | AAGAUAAUUGGAGAAAAAU | AUUUUUCUCCAAUUAUCUU | Rat, Ms, GP, Chmp | [1755-1773] |
| 229 | AAAUUCUGCCCUUCAUUCA | UGAAUGAAGGGCAGAAUUU | Chmp | [1420-1438] |
| 230 | GAAUGCCCUCAACUUCUCA | UGAGAAGUUGAGGGCAUUC | Chmp | [614-632] |
| 231 | CUUUCAGCUGAAAACUUUU | AAAAGUUUUCAGCUGAAAG | Chmp | [1966-1984]+ |
| 232 | GAUUUUCAAAACCGGCAGA | UCUGCCGGUUUUGAAAAUC | Chmp | [1000-1018] |
| 233 | GGUGUUACUAUCUGUAUUU | AAAUACAGAUAGUAACACC | Chin, GP, Chmp | [564-582] |
| 234 | CUUCCGUUGGUUUGCAGAU | AUCUGCAAACCAACGGAAG | Chmp | [1643-1661] |
| 235 | UAAGAGUUUCUAACUAUGA | UCAUAGUUAGAAACUCUUA | Chmp | [784-802] |
| 236 | UGUUGAACUGAAUGCAGCA | UGCUGCAUUCAGUUCAACA | GP, Chmp | [653-671] |
| 237 | UUAGAUGCACACUGUUGAU | AUCAACAGUGUGCAUCUAA | Rat, Chmp | [2231-2249] |
| 238 | GAAUGCAGCAAGAUACCGA | UCGGUAUCUUGCUGCAUUC | GP, Chmp | [662-680] |
| 239 | GCACUGAAUUCAAGACUGU | ACAGUCUUGAAUUCAGUGC | Chmp | [1779-1797] |
| 240 | CAUAUUACCUGUGGUGUUA | UAACACCACAGGUAAUAUG | Chmp | [552-570] |
| 241 | AUUUAAAAUGGAAAUGUGA | UCACAUUUCCAUUUUAAAU | Chmp | [2100-2118] |
| 242 | GUGAGGAGCUGAACUUGCU | AGCAAGUUCAGCUCCUCAC | Chmp | [2277-2295] |
| 243 | GAGGAAUCACUGAACUAUG | CAUAGUUCAGUGAUUCCUC | Chmp | [1491-1509] |
| 244 | CUGGAAACCAUACAAGCUU | AAGCUUGUAUGGUUUCCAG | Chin, GP, Chmp | [1583-1601] |
| 245 | ACUACUGCCUCCAUCUAGU | ACUAGAUGGAGGCAGUAGU | Chmp | [1391-1409] |
| 246 | CUUUUGGAAGUCCAUUUGA | UCAAAUGGACUUCCAAAAG | Chmp | [1474-1492] |
| 247 | AAGCAGGACUCUAAAGAAG | CUUCUUUAGAGUCCUGCUU | Chmp | [1991-2009] |
| 248 | AUUACAUGUUUAAUCUGGA | UCCAGAUUAAACAUGUAAU | Chmp | [2142-2160] |
| 249 | GAUUCAAGAACUCCCUAGU | ACUAGGGAGUUCUUGAAUC | Chmp | [2260-2278] |
| 250 | AUAAACUGAGUAACCAGAA | UUCUGGUUACUCAGUUUAU | Chmp | [1909-1927] |
| 251 | AUGUCAUGGAAAUCCGAAU | AUUCGGAUUUCCAUGACAU | GP, Chin, Chmp | [1198-1216] |
| 252 | AGUAUCAAACUAAUUUAGA | UCUAAAUUAGUUUGAUACU | Chmp | [880-898] |
| 253 | CUUUUCAGUUCUCAAAUUA | UAAUUUGAGAACUGAAAAG | Chmp | [2201-2219] |
| 254 | GUGCCGAAAGACUUUACAG | CUGUAAAGUCUUUCGGCAC | Chin, Chmp | [1123-1141] |
| 255 | AAUCUGGAAACCAAAGAGA | UCUCUUUGGUUUCCAGAUU | Chmp | [2153-2171] |
| 256 | AUUGAUGGUCCUUUUGGAA | UUCCAAAAGGACCAUCAAU | Chmp | [1464-1482] |
| 257 | UUCACUUUUCAGUUCUCAA | UUGAGAACUGAAAAGUGAA | Chmp | [2197-2215] |
| 258 | CAUAUGGGACAAGAUUUGA | UCAAAUCUUGUCCCAUAUG | | [1933-1951] |
| 259 | AUAUCUUCUGGUAUACUCA | UGAGUAUACCAGAAGAUAU | Rat, Ms, Chin, GP, Chmp | [802-820] |
| 260 | UACCGAGAUGAGGAUCCUA | UAGGAUCCUCAUCUCGGUA | GP, Chmp | [675-693] |
| 261 | AACUUCUCAGUGAAUUACA | UGUAAUUCACUGAGAAGUU | Chmp | [624-642] |
| 262 | GUGGUGGUGCUAUUCCUCA | UGAGGAAUAGCACCACCAC | Chmp | [741-759] |
| 263 | UCAUGCACUGAAUUCAAGA | UCUUGAAUUCAGUGCAUGA | Chmp | [1775-1793] |
| 264 | AAGACUCUUCAUAAACUGA | UCAGUUUAUGAAGAGUCUU | Chmp | [1899-1917] |
| 265 | GUUGUGGACCCAAUUCACU | AGUGAAUUGGGUCCACAAC | Chmp | [1876-1894] |
| 266 | UACCUCCGAGGAUCACAGA | UCUGUGAUCCUCGGAGGUA | Chmp | [483-501] |
| 267 | CAGUAUAUUACUCUACAUU | AAUGUAGAGUAAUAUACUG | Chmp | [1248-1266] |
| 268 | UGGACAGAACGAUUUCGAG | CUCGAAAUCGUUCUGUCCA | Chmp | [1368-1386] |
| 269 | UACUAUCUGUAUUUUCUCA | UGAGAAAAUACAGAUAGUA | Chin, GP, Chmp | [569-587] |
| 270 | CAUUUCGGUCAUAAGUCAU | AUGACUUAUGACCGAAAUG | Chmp | [1172-1190] |
| 271 | CACAUUACAUGUUUAAUCU | AGAUUAAACAUGUAAUGUG | Chmp | [2139-2157] |
| 272 | GUAUAUCCGGAGCAAUAAG | CUUAUUGCUCCGGAUAUAC | Chmp | [1142-1160] |
| 273 | AGUCAUCCCUCAGAUGUCA | UGACAUCUGAGGGAUGACU | Chmp | [1185-1203] |
| 274 | GCAGCAAGAUACCGAGAUG | CAUCUCGGUAUCUUGCUGC | GP, Chmp | [666-684] |
| 275 | CAUUUACCCUCACAAUGUG | CACAUUGUGAGGGUAAAUG | GP, Chmp | [1297-1315] |
| 276 | AAAUUUGUGAAGAUUUGUA | UACAAAUCUUCACAAAUUU | Chmp | [1032-1050] |
| 277 | UAGUCAAGACUCCGAAAUU | AAUUUCGGAGUCUUGACUA | Chmp | [1406-1424] |
| 278 | ACUUUGAAACUCAGCGGAA | UUCCGCUGAGUUUCAAAGU | Chmp | [2030-2048] |
| 279 | GAAUUCAAGACUGUUUAUA | UAUAAACAGUCUUGAAUUC | | [1784-1802] |
| 280 | UUAGAAAAUCAUCCAUUUA | UAAAUGGAUGAUUUUCUAA | Chmp | [1284-1302] |
| 281 | GAAUGGUCAAAGAAAAUUU | AAAUUUUCUUUGACCAUUC | GP, Chmp | [1213-1231] |
| 282 | CCUCAUGAUCACAGCCUCU | AGAGGCUGUGAUCAUGAGG | Chmp | [755-773] |
| 283 | AAUAGUAAGGUUUUCUUAU | AUAAGAAAACCUUACUAUU | Chmp | [2073-2091] |
| 284 | AAGAAAACUGUUAGAUGCA | UGCAUCUAACAGUUUUCUU | Chmp | [2221-2239] |
| 285 | UGUACUGUGCCGAAAGACU | AGUCUUUCGGCACAGUACA | Chin, Chmp | [1117-1135] |
| 286 | CUUACCAGAGUAUUUCUCA | UGAGAAAUACUCUGGUAAG | Chmp | [953-971] |
| 287 | AUGAGUGCAAUUUCUAAGA | UCUUAGAAAUUGCACUCAU | Chmp | [2012-2030] |
| 288 | AAGCUUAGAAGACUAUACU | AGUAUAGUCUUCUAAGCUU | Chmp | [1596-1614] |
| 289 | GAUGACCGUCACAUUACAU | AUGUAAUGUGACGGUCAUC | | [2130-2148] |
| 290 | GAUGAUUCACUUUUCAGUU | AACUGAAAAGUGAAUCAUC | Chmp | [2192-2210] |
| 291 | GGAAACCAUACAAGCUUAG | CUAAGCUUGUAUGGUUUCC | Chin, GP, Chmp | [1585-1603] |
| 292 | AACCGGCAGAGUUUACCCA | UGGGUAAACUCUGCCGGUU | Chmp | [1009-1027] |
| 293 | CAACUGAAACCAAAGCAAC | GUUGCUUUGGUUUCAGUUG | Chmp | [1318-1336] |
| 294 | UUACCUGUGGUGUUACUAU | AUAGUAACACCACAGGUAA | Chmp | [556-574] |
| 295 | ACUUCUCAGUGAAUUACAG | CUGUAAUUCACUGAGAAGU | | [625-643] |
| 296 | UUUGCAGAUUUACUCUGUA | UACAGAGUAAAUCUGCAAA | Chmp | [1653-1671] |
| 297 | UUGAGGAAUCACUGAACUA | UAGUUCAGUGAUUCCUCAA | Chmp | [1489-1507] |
| 298 | AUGGUCAAAGAAAAUUUUA | UAAAAUUUUCUUUGACCAU | GP, Chmp | [1215-1233] |
| 299 | CACAAAUUUGUGAAGAUUU | AAAUCUUCACAAAUUUGUG | Chmp | [1029-1047] |
| 300 | AAUAAGAGUUUCUAACUAU | AUAGUUAGAAACUCUUAUU | Chmp | [782-800] |
| 301 | AAAAUAGUAGGAGACUGGA | UCCAGUCUCCUACUAUUUU | Chin, GP, Chmp | [1353-1371] |
| 302 | GAACUUGCUCAAUCUAAGG | CCUUAGAUUGAGCAAGUUC | Chmp | [2287-2305] |
| 303 | UGGAUUUCUGGACCUUUGU | ACAAAGGUCCAGAAAUCCA | Rat, Ms, GP, Chin, Chmp | [1095-1113] |
| 304 | UGAACCUUUCCCUGAAGGA | UCCUUCAGGGAAAGGUUCA | Chmp | [983-1001] |
| 305 | GUUACUAUCUGUAUUUUCU | AGAAAAUACAGAUAGUAAC | Chin, GP, Chmp | [567-585] |
| 306 | AAACACCUCUGCCUGUUCA | UGAACAGGCAGAGGUGUUU | | [282-300] |
| 307 | UUAAAAGAAAACUGUUAGA | UCUAACAGUUUUCUUUUAA | Chmp | [2217-2235] |
| 308 | AUAUAACAGAGGAAAAACA | UGUUUUUCCUCUGUUAUAU | | [1844-1862] |
| 309 | AACACCUCUGCCUGUUCAU | AUGAACAGGCAGAGGUGUU | | [283-301] |
| 310 | GAUGAGGAUCCUAGAAAAC | GUUUUCUAGGAUCCUCAUC | GP, Chmp | [681-699] |
| 311 | GAUUUUCAUGGUGGAUUCA | UGAAUCCACCAUGAAAAUC | | [2247-2265] |
| 312 | GAAGGAUUUUCAAAACCGG | CCGGUUUUGAAAAUCCUUC | Chmp | [996-1014] |
| 313 | AUUAGAAAAUCAUCCAUUU | AAAUGGAUGAUUUUCUAAU | Chmp | [1283-1301] |
| 314 | ACCUGGUCAGUAUAUUACU | AGUAAUAUACUGACCAGGU | Chmp | [1241-1259] |
| 315 | ACUCAUAUGGGACAAGAUU | AAUCUUGUCCCAUAUGAGU | | [1930-1948] |
| 316 | AAGAUACCGAGAUGAGGAU | AUCCUCAUCUCGGUAUCUU | GP, Chmp | [671-689] |
| 317 | AGAAUAGUAAGGUUUUCUU | AAGAAAACCUUACUAUUCU | Chmp | [2071-2089] |
| 318 | ACUGGAAACCAUACAAGCU | AGCUUGUAUGGUUUCCAGU | Rat, Chin, GP, Chmp | [1582-1600] |
| 319 | UCUGGUGAAUGCCCUCAAC | GUUGAGGGCAUUCACCAGA | Chmp | [608-626] |
| 320 | AUCUUAAAAUAGUAGGAGA | UCUCCUACUAUUUUAAGAU | Chin, GP, Chmp | [1348-1366] |
| 321 | GAAUAUUUCCUUACCAGAG | CUCUGGUAAGGAAAUAUUC | Chmp | [944-962] |
| 322 | GCAAGAUACCGAGAUGAGG | CCUCAUCUCGGUAUCUUGC | GP, Chmp | [669-687] |
| 323 | GGUGAAUGCCCUCAACUUC | GAAGUUGAGGGCAUUCACC | Chmp | [611-629] |
| 324 | GAUUUACUACUGCCUCCAU | AUGGAGGCAGUAGUAAAUC | Rat, Chmp | [1386-1404] |
| 325 | AGAUUGUUGGAUAAAAGCA | UGCUUUUAUCCAACAAUCU | Ms, Chmp | [525-543] |
| 326 | ACUUCUCUUCACAACUGUU | AACAGUUGUGAAGAGAAGU | Chmp | [698-716] |
| 327 | UAUGCAAUAAGAGUUUCUA | UAGAAACUCUUAUUGCAUA | Rat, Chmp | [777-795] |
| 328 | GAUCCUAGAAAACUUCUCU | AGAGAAGUUUUCUAGGAUC | GP, Chmp | [687-705] |
| 329 | ACUGUGCCGAAAGACUUUA | UAAAGUCUUUCGGCACAGU | Chin, Chmp | [1120-1138] |
| 330 | CCAAUUCACUAUCCAAGAC | GUCUUGGAUAGUGAAUUGG | Chmp | [1885-1903] |
| 331 | AAUCAUCCAUUUACCCUCA | UGAGGGUAAAUGGAUGAUU | Chmp | [1290-1308] |
| 332 | AUGGAAAUCCGAAUGGUCA | UGACCAUUCGGAUUUCCAU | GP, Chin, Chmp | [1203-1221] |
| 333 | CUUACCUCCGAGGAUCACA | UGUGAUCCUCGGAGGUAAG | Chmp | [481-499] |
| 334 | GCUAUUCCUCAUGAUCACA | UGUGAUCAUGAGGAAUAGC | Chmp | [749-767] |
| 335 | CAGAUGUCAUGGAAAUCCG | CGGAUUUCCAUGACAUCUG | GP, Chin, Chmp | [1195-1213] |
| 336 | AAAUGCAAGAUUGAACAAA | UUUGUUCAAUCUUGCAUUU | Chmp | [2346-2364] |
| 337 | UAUUUGAUGUGAUGAUUCA | UGAAUCAUCACAUCAAAUA | Chmp | [2182-2200] |
| 338 | CUGGUGAAUGCCCUCAACU | AGUUGAGGGCAUUCACCAG | Chmp | [609-627] |
| 339 | AAUGGAAAUGUGAGAAUGU | ACAUUCUCACAUUUCCAUU | Chmp | [2106-2124] |
| 340 | GGGCUUCCACUCAGUCUUU | AAAGACUGAGUGGAAGCCC | | [103-121] |
| 341 | UGCAAGAUUGAACAAAAUU | AAUUUUGUUCAAUCUUGCA | Chmp | [2349-2367] |
| 342 | AACCAAAGAGACCCUGAAG | CUUCAGGGUCUCUUUGGUU | Chmp | [2161-2179] |
| 343 | AAUGGUCAAAGAAAAUUUU | AAAAUUUUCUUUGACCAUU | GP, Chmp | [1214-1232] |
| 344 | UAAUUGGAGAAAAAUAUCA | UGAUAUUUUUCUCCAAUUA | Ms, GP, Chmp | [1759-1777] |
| 345 | GAAAAAUAUCAUGCACUGA | UCAGUGCAUGAUAUUUUUC | Chmp | [1767-1785] |
| 346 | AACUUCUCUUCACAACUGU | ACAGUUGUGAAGAGAAGUU | Chmp | [697-715] |
| 347 | AUAAAAGCAGAACAUUCCA | UGGAAUGUUCUGCUUUUAU | Chmp | [535-553] |
| 348 | GAGUUUCUAACUAUGAUAU | AUAUCAUAGUUAGAAACUC | Chmp | [787-805] |
| 349 | AUAGUAAGGUUUUCUUAUU | AAUAAGAAAACCUUACUAU | Chmp | [2074-2092] |
| 350 | GAAGGAAUGAGUGCAAUUU | AAAUUGCACUCAUUCCUUC | Chmp | [2006-2024] |
| 351 | UUAAAUUGUUUUUGGUUGA | UCAACCAAAAACAAUUUAA | Chmp | [2325-2343] |
| 352 | UGGAAGAGCCCAGAUUCCA | UGGAAUCUGGGCUCUUCCA | Chmp | [1051-1069] |
| 353 | UGAAGAAUAUUUGAUGUGA | UCACAUCAAAUAUUCUUCA | Chmp | [2175-2193] |
| 354 | UAAAUUGUUUUUGGUUGAA | UUCAACCAAAAACAAUUUA | Chmp | [2326-2344] |
| 355 | UCUAAGACUUUGAAACUCA | UGAGUUUCAAAGUCUUAGA | Chmp | [2024-2042] |
| 356 | AAUCUAGAAAUUAUCCCAA | UUGGGAUAAUUUCUAGAUU | Chin, Chmp | [1438-1456] |
| 357 | AAAGCAAGACCUGGUCAGU | ACUGACCAGGUCUUGCUUU | GP, Chmp | [1233-1251] |
| 358 | CAAAACCGGCAGAGUUUAC | GUAAACUCUGCCGGUUUUG | Chmp | [1006-1024] |
| 359 | AGAACAGACCUGACUAUGU | ACAUAGUCAGGUCUGUUCU | Chin, GP, Chmp | [1696-1714] |
| 360 | UUUCAAAACCGGCAGAGUU | AACUCUGCCGGUUUUGAAA | Chmp | [1003-1021] |
| 361 | AACGAUUUCGAGAUUUACU | AGUAAAUCUCGAAAUCGUU | Chmp | [1375-1393] |
| 362 | UUUGCAUCAAUACUCAACA | UGUUGAGUAUUGAUGCAAA | Chmp | [1551-1569] |
| 363 | UAAAGAGUCUUUCAGCUGA | UCAGCUGAAAGACUCUUUA | Chmp | [1958-1976] |
| 364 | UUUUCAUGGUGGAUUCAAG | CUUGAAUCCACCAUGAAAA | | [2249-2267] |
| 365 | AAGAAAAUUUUAAAGCAAG | CUUGCUUUAAAAUUUUCUU | GP, Chmp | [1222-1240] |
| 366 | CUGCUGACGUUGCAUGUUU | AAACAUGCAACGUCAGCAG | Chmp | [846-864] |
| 367 | GAAGAGCCCAGAUUCCAAG | CUUGGAAUCUGGGCUCUUC | Chmp | [1053-1071] |
| 368 | AAUUCAAGACUGUUUAUAG | CUAUAAACAGUCUUGAAUU | | [1785-1803] |
| 369 | AACAGUUGGUGUUUUCUGU | ACAGAAAACACCAACUGUU | Chmp | [1859-1877] |
| 370 | GUAUUUCUCAGAACAUUUU | AAAAUGUUCUGAGAAAUAC | Chmp | [962-980] |
| 371 | GUCACAUUACAUGUUUAAU | AUUAAACAUGUAAUGUGAC | Chmp | [2137-2155] |
| 372 | UUUUGGUUGAACAAAUGCA | UGCAUUUGUUCAACCAAAA | Chmp | [2334-2352] |
| 373 | GUAUUUUCUCAGGCGUGCA | UGCACGCCUGAGAAAAUAC | Chmp | [577-595] |
| 374 | GUUCCUCUUUAAAUUGUUU | AAACAAUUUAAAGAGGAAC | Chmp | [2317-2335] |
| 375 | UGAAGUAUCAAACUAAUUU | AAAUUAGUUUGAUACUUCA | Chmp | [877-895] |
| 376 | CUUGCUGUAUAACCAAGGG | CCCUUGGUUAUACAGCAAG | Chmp | [341-359] |
| 377 | AUUCUGCCCUUCAUUCAAU | AUUGAAUGAAGGGCAGAAU | Rat, Chin, Chmp | [1422-1440] |
| 378 | UAUGUUGCAUAACAAGUUU | AAACUUGUUAUGCAACAUA | Chmp | [1670-1688] |
| 379 | UUUCCUUACCAGAGUAUUU | AAAUACUCUGGUAAGGAAA | Chmp | [949-967] |
| 380 | GCUUCCACUCAGUCUUUGA | UCAAAGACUGAGUGGAAGC | | [105-123] |
| 381 | AAUAGUAGGAGACUGGACA | UGUCCAGUCUCCUACUAUU | Chin, GP, Chmp | [1355-1373] |
| 382 | UGUUCCUCUUUAAAUUGUU | AACAAUUUAAAGAGGAACA | Chmp | [2316-2334] |
| 383 | CAUGCUGCUGACGUUGCAU | AUGCAACGUCAGCAGCAUG | Chmp | [842-860] |
| 384 | UCCUUACCAGAGUAUUUCU | AGAAAUACUCUGGUAAGGA | Chmp | [951-969] |
| 385 | UGAAUACAAUAAAGAGUCU | AGACUCUUUAUUGUAUUCA | Chmp | [1949-1967] |
| 386 | CCAUACAAGCUUAGAAGAC | GUCUUCUAAGCUUGUAUGG | Chin, GP, Chmp | [1590-1608] |
| 387 | UACCAGAGUAUUUCUCAGA | UCUGAGAAAUACUCUGGUA | Chmp | [955-973] |
| 388 | AGAACAUUCCAUAUUACCU | AGGUAAUAUGGAAUGUUCU | Chmp | [543-561] |
| 389 | AAAACUUCUCUUCACAACU | AGUUGUGAAGAGAAGUUUU | Chmp | [695-713] |
| 390 | UACAAGCUUAGAAGACUAU | AUAGUCUUCUAAGCUUGUA | Chmp | [1593-1611] |
| 391 | AGUAUUUCUCAGAACAUUU | AAAUGUUCUGAGAAAUACU | Chmp | [961-979] |
| 392 | AAUUAUCCCAAGCUGUAUA | UAUACAGCUUGGGAUAAUU | Chmp | [1446-1464] |
| 393 | GAUUGUUGGAUAAAAGCAG | CUGCUUUUAUCCAACAAUC | Chmp | [526-544] |
| 394 | AAAUUUUAAAGCAAGACCU | AGGUCUUGCUUUAAAAUUU | GP, Chmp | [1226-1244] |
| 395 | UUCAAAACCGGCAGAGUUU | AAACUCUGCCGGUUUUGAA | Chmp | [1004-1022] |
| 396 | AAGUCCAUUUGAGGAAUCA | UGAUUCCUCAAAUGGACUU | Chin, Chmp | [1481-1499] |
| 397 | GCUUAGAAGACUAUACUUU | AAAGUAUAGUCUUCUAAGC | Chmp | [1598-1616] |
| 398 | AACCAUACAAGCUUAGAAG | CUUCUAAGCUUGUAUGGUU | Chin, GP, Chmp | [1588-1606] |
| 399 | CUUUGUUGAACUGAAUGCA | UGCAUUCAGUUCAACAAAG | | [650-668] |
| 400 | UCAUCUUAAAAUAGUAGGA | UCCUACUAUUUUAAGAUGA | Chmp | [1346-1364] |
| 401 | AAUUUGUGAAGAUUUGUAU | AUACAAAUCUUCACAAAUU | Chmp | [1033-1051] |
| 402 | AGGAGGGCUGCUGAAGUAU | AUACUUCAGCAGCCCUCCU | GP, Chmp | [866-884] |
| 403 | GUGUUACUAUCUGUAUUUU | AAAAUACAGAUAGUAACAC | Chin, GP, Chmp | [565-583] |
| 404 | CAUUAGAAAAUCAUCCAUU | AAUGGAUGAUUUUCUAAUG | Chmp | [1282-1300] |
| 405 | UUCUGUUGUGGACCCAAUU | AAUUGGGUCCACAACAGAA | Chmp | [1872-1890] |
| 406 | ACUAUCCAAGACUCUUCAU | AUGAAGAGUCUUGGAUAGU | Chmp | [1892-1910] |
| 407 | UCUUUAAAUUGUUUUUGGU | ACCAAAAACAAUUUAAAGA | Chmp | [2322-2340] |
| 408 | ACUAUGAUAUCUUCUGGUA | UACCAGAAGAUAUCAUAGU | Chmp | [796-814] |
| 409 | AUCUAAGGCUGAUUGUCGU | ACGACAAUCAGCCUUAGAU | Chmp | [2298-2316] |
| 410 | UUACUACUGCCUCCAUCUA | UAGAUGGAGGCAGUAGUAA | Chmp | [1389-1407] |
| 411 | UUACCAGAGUAUUUCUCAG | CUGAGAAAUACUCUGGUAA | Chmp | [954-972] |
| 412 | GGUGCUAUUCCUCAUGAUC | GAUCAUGAGGAAUAGCACC | Chmp | [746-764] |
| 413 | AACAGACCUGACUAUGUCA | UGACAUAGUCAGGUCUGUU | Chin, GP, Chmp | [1698-1716] |
| 414 | CAAAUUUGUGAAGAUUUGU | ACAAAUCUUCACAAAUUUG | Chmp | [1031-1049] |
| 415 | AGCCUUAUCCUUUUACCCA | UGGGUAAAAGGAUAAGGCU | Chin, GP | [444-462] |
| 416 | CUCCAUUUGCAUCAAUACU | AGUAUUGAUGCAAAUGGAG | Chmp | [1546-1564] |
| 417 | CCUAGUGAGGAGCUGAACU | AGUUCAGCUCCUCACUAGG | Chmp | [2273-2291] |
| 418 | UUAAAAUGGAAAUGUGAGA | UCUCACAUUUCCAUUUUAA | Chmp | [2102-2120] |
| 419 | ACGUCCUCGGUGGAAACUU | AAGUUUCCACCGAGGACGU | Chmp | [1805-1823] |
| 420 | AAACUGAGUAACCAGAACA | UGUUCUGGUUACUCAGUUU | | [1911-1929] |
| 421 | UGUCCAACUGAAACCAAAG | CUUUGGUUUCAGUUGGACA | Chmp | [1314-1332] |
| 422 | GUGAAUGCCCUCAACUUCU | AGAAGUUGAGGGCAUUCAC | Chmp | [612-630] |
| 423 | UAGUAAGGUUUUCUUAUUU | AAAUAAGAAAACCUUACUA | Chmp | [2075-2093] |
| 424 | AACCUUUCCCUGAAGGAUU | AAUCCUUCAGGGAAAGGUU | Chmp | [985-1003] |
| 425 | CAGUCUUAACCGAACCAGC | GCUGGUUCGGUUAAGACUG | Chmp | [920-938] |
| 426 | GCUGCAUCAGUCUUAACCG | CGGUUAAGACUGAUGCAGC | Chmp | [913-931] |
| 427 | UAUCUGCAUUAGAAAAUCA | UGAUUUUCUAAUGCAGAUA | Chmp | [1276-1294] |
| 428 | GGUUUUCUUAUUUAUGAUU | AAUCAUAAAUAAGAAAACC | Chmp | [2081-2099] |
| 429 | UAUCUUCUGGUAUACUCAU | AUGAGUAUACCAGAAGAUA | Chin, GP, Chmp | [803-821] |
| 430 | GUCAAGACUCCGAAAUUCU | AGAAUUUCGGAGUCUUGAC | Chmp | [1408-1426] |
| 431 | UUGUUUUUGGUUGAACAAA | UUUGUUCAACCAAAAACAA | Chmp | [2330-2348] |
| 432 | UUCAUGGUGGAUUCAAGAA | UUCUUGAAUCCACCAUGAA | | [2251-2269] |
| 433 | UUUCUGUUGUGGACCCAAU | AUUGGGUCCACAACAGAAA | Chmp | [1871-1889] |
| 434 | CAUGGAAAUCCGAAUGGUC | GACCAUUCGGAUUUCCAUG | GP, Chin, Chmp | [1202-1220] |
| 435 | UCAAGACUCCGAAAUUCUG | CAGAAUUUCGGAGUCUUGA | Chmp | [1409-1427] |
| 436 | UGUAUGGAAGAGCCCAGAU | AUCUGGGCUCUUCCAUACA | Chmp | [1047-1065] |
| 437 | AGACUGGACAGAACGAUUU | AAAUCGUUCUGUCCAGUCU | Chmp | [1364-1382] |
| 438 | CACCUCUGCCUGUUCAUCU | AGAUGAACAGGCAGAGGUG | | [285-303] |
| 439 | AGUCUUAACCGAACCAGCU | AGCUGGUUCGGUUAAGACU | Chmp | [921-939] |
| 440 | AUUUGAAUACAAUAAAGAG | CUCUUUAUUGUAUUCAAAU | Chmp | [1946-1964] |
| 441 | ACCUGUGGUGUUACUAUCU | AGAUAGUAACACCACAGGU | Chmp | [558-576] |
| 442 | ACAGAACGAUUUCGAGAUU | AAUCUCGAAAUCGUUCUGU | Chmp | [1371-1389] |
| 443 | UCUGGUAUACUCAUAACCU | AGGUUAUGAGUAUACCAGA | Chin, GP, Chmp | [808-826] |
| 444 | UUCUUAACCUCAACUGCAG | CUGCAGUUGAGGUUAAGAA | Chmp | [427-445] |
| 445 | CCAAAGAAUAGUAAGGUUU | AAACCUUACUAUUCUUUGG | Chmp | [2067-2085] |
| 446 | UUGUAUGGAAGAGCCCAGA | UCUGGGCUCUUCCAUACAA | Chmp | [1046-1064] |
| 447 | UGGUAUACUCAUAACCUCU | AGAGGUUAUGAGUAUACCA | Chin, GP, Chmp | [810-828] |
| 448 | AAGUAUCAAACUAAUUUAG | CUAAAUUAGUUUGAUACUU | Chmp | [879-897] |
| 449 | UUGUGAAGAUUUGUAUGGA | UCCAUACAAAUCUUCACAA | Chmp | [1036-1054] |
| 450 | AUCUGCAUUAGAAAAUCAU | AUGAUUUUCUAAUGCAGAU | Chmp | [1277-1295] |
| 451 | CAAUAAAGAGUCUUUCAGC | GCUGAAAGACUCUUUAUUG | Chmp | [1955-1973] |
| 452 | GAUUGUCGUGUUCCUCUUU | AAAGAGGAACACGACAAUC | Chmp | [2308-2326] |
| 453 | ACUUUUCAGUUCUCAAAUU | AAUUUGAGAACUGAAAAGU | Chmp | [2200-2218] |
| 454 | GUUGAUUUUCAUGGUGGAU | AUCCACCAUGAAAAUCAAC | | [2244-2262] |
| 455 | UACUCAUAACCUCUUCUUU | AAAGAAGAGGUUAUGAGUA | Chin, GP, Chmp | [815-833] |
| 456 | AGAGUAUCACUACCUCCAC | GUGGAGGUAGUGAUACUCU | Chin, GP, Chmp | [362-380] |
| 457 | GACUGGACAGAACGAUUUC | GAAAUCGUUCUGUCCAGUC | Chmp | [1365-1383] |
| 458 | CCUCAACUGCAGCCUUAUC | GAUAAGGCUGCAGUUGAGG | Chin, Chmp | [434-452] |
| 459 | UAAUCUGGAAACCAAAGAG | CUCUUUGGUUUCCAGAUUA | Chmp | [2152-2170] |
| 460 | AAUAUUUCCUUACCAGAGU | ACUCUGGUAAGGAAAUAUU | Chmp | [945-963] |
| 461 | AGAAAUUAUCCCAAGCUGU | ACAGCUUGGGAUAAUUUCU | Chmp | [1443-1461] |
| 462 | CUUCUCAGUGAAUUACAGU | ACUGUAAUUCACUGAGAAG | | [626-644] |
| 463 | ACAUAUGCAAUAAGAGUUU | AAACUCUUAUUGCAUAUGU | Chmp | [774-792] |
| 464 | CAGACCUGACUAUGUCAAC | GUUGACAUAGUCAGGUCUG | Chmp | [1700-1718] |
| 465 | GGUUAAACACCUCUGCCUG | CAGGCAGAGGUGUUUAACC | Ms | [278-296] |
| 466 | AAUGCAGCAAGAUACCGAG | CUCGGUAUCUUGCUGCAUU | GP, Chmp | [663-681] |
| 467 | GAACAGACCUGACUAUGUC | GACAUAGUCAGGUCUGUUC | Chin, GP, Chmp | [1697-1715] |
| 468 | GUUUACCCAGCACAAAUUU | AAAUUUGUGCUGGGUAAAC | Chmp | [1019-1037] |
| 469 | UCAUGAUCACAGCCUCUAC | GUAGAGGCUGUGAUCAUGA | Chmp | [757-775] |
| 470 | AAAUAUCAUGCACUGAAUU | AAUUCAGUGCAUGAUAUUU | Chmp | [1770-1788] |
| 471 | GAAGACUUUGUUGAACUGA | UCAGUUCAACAAAGUCUUC | | [645-663] |
| 472 | AACAGAUGGGAUACAGAAG | CUUCUGUAUCCCAUCUGUU | Rat, GP, Chmp | [1739-1757] |
| 473 | AAGCAGGAGAACCAGGAGA | UCUCCUGGUUCUCCUGCUU | Chmp | [509-527] |
| 474 | AUUUCUCAGAACAUUUUCA | UGAAAAUGUUCUGAGAAAU | Chmp | [964-982] |
| 475 | AUGAUCACAGCCUCUACAU | AUGUAGAGGCUGUGAUCAU | Chmp | [759-777] |
| 476 | GUUAAACACCUCUGCCUGU | ACAGGCAGAGGUGUUUAAC | | [279-297] |
| 477 | GACCUUUGUGCCUGUACUG | CAGUACAGGCACAAAGGUC | GP, Chin, Chmp | [1105-1123] |
| 478 | AAAUUAUCCCAAGCUGUAU | AUACAGCUUGGGAUAAUUU | Chmp | [1445-1463] |
| 479 | CUUUAAAUUGUUUUUGGUU | AACCAAAAACAAUUUAAAG | Chmp | [2323-2341] |
| 480 | UCUACAUAUGCAAUAAGAG | CUCUUAUUGCAUAUGUAGA | Chmp | [771-789] |
| 481 | AGCAAGAUACCGAGAUGAG | CUCAUCUCGGUAUCUUGCU | GP, Chmp | [668-686] |
| 482 | UUUCCCUGAAGGAUUUUCA | UGAAAAUCCUUCAGGGAAA | Chmp | [989-1007] |
| 483 | ACAAAUUUGUGAAGAUUUG | CAAAUCUUCACAAAUUUGU | Chmp | [1030-1048] |
| 484 | GGUGGAUUCAAGAACUCCC | GGGAGUUCUUGAAUCCACC | Chmp | [2256-2274] |
| 485 | UACUCUACAUUGUCCCAGU | ACUGGGACAAUGUAGAGUA | Chmp | [1256-1274] |
| 486 | CAGUCCUUCCGUUGGUUUG | CAAACCAACGGAAGGACUG | Chmp | [1638-1656] |
| 487 | AUCUGUUCUUAACCUCAAC | GUUGAGGUUAAGAACAGAU | Chmp | [422-440] |
| 488 | UAUUUAAAAUGGAAAUGUG | CACAUUUCCAUUUUAAAUA | Chmp | [2099-2117] |
| 489 | AGAUAUCCAGUCCUUCCGU | ACGGAAGGACUGGAUAUCU | Chmp | [1631-1649] |
| 490 | AAUACAAUAAAGAGUCUUU | AAAGACUCUUUAUUGUAUU | Chmp | [1951-1969] |
| 491 | AUAACAGAGGAAAAACAGU | ACUGUUUUUCCUCUGUUAU | | [1846-1864] |
| 492 | UACUUUAUUUGGGUAUGCA | UGCAUACCCAAAUAAAGUA | Chmp | [1611-1629] |
| 493 | GAGGAGCUGAACUUGCUCA | UGAGCAAGUUCAGCUCCUC | Chmp | [2279-2297] |
| 494 | UUAAUCUGGAAACCAAAGA | UCUUUGGUUUCCAGAUUAA | Chmp | [2151-2169] |
| 495 | UAUCACUACCUCCACCAGA | UCUGGUGGAGGUAGUGAUA | Chin, GP, Chmp | [366-384] |
| 496 | UCUGGAAAACCUUCUUGCU | AGCAAGAAGGUUUUCCAGA | Chmp | [328-346] |
| 497 | UCAAGAACUCCCUAGUGAG | CUCACUAGGGAGUUCUUGA | Chmp | [2263-2281] |
| 498 | UUGGAAGUCCAUUUGAGGA | UCCUCAAAUGGACUUCCAA | Rat, Chin, Chmp | [1477-1495] |
| 499 | CCUUUUACCCAUGUGCCGA | UCGGCACAUGGGUAAAAGG | Chin | [452-470] |
| 500 | GUAUGCAGAGAUAUCCAGU | ACUGGAUAUCUCUGCAUAC | Chmp | [1623-1641] |

TABLE A3
NOX1 19-mer oligomer pairs (NADPH oxidase 1)

| | | | | Human- | Human- |
|---|---|---|---|---|---|
| | | | | 21614529 | 7669509 |
| No. | Sense siRNA | AntiSense siRNA | Other Sp | ORF: 207-1901 | ORF: 71-1618 |
| 1 | CUCUCUGCUUGAAUUUUAA | UUAAAAUUCAAGCAGAGAG | | [376-394] | [240-258] |
| 2 | GGAGUUAUUUUGAAGUCUU | AAGACUUCAAAAUAACUCC | | [799-817] | [663-681] |
| 3 | GGCUGAAUGUUUUCCUGUU | AACAGGAAAACAUUCAGCC | | [265-283] | [129-147] |
| 4 | GCAAAGUGGGUUUUCUAAA | UUUAGAAAACCCACUUUGC | | [1594-1612] | — |
| 5 | GCAGACCACAACCUCAAAA | UUUUGAGGUUGUGGUCUGC | GP | [1476-1494] | [1340-1358] |
| 6 | GGACAGAAAAUCUCAUAAG | CUUAUGAGAUUUUCUGUCC | Rat | [1288-1306] | [1152-1170] |
| 7 | CCGGAGGAGUUAUUUUGAA | UUCAAAAUAACUCCUCCGG | | [794-812] | [658-676] |
| 8 | ACAUGUUUACUGUCACUUU | AAAGUGACAGUAAACAUGU | | [2487-2505] | — |
| 9 | GUAGGGUGACUUUUUAAAU | AUUUAAAAAGUCACCCUAC | | [2371-2389] | — |
| 10 | CCAGGUGAUUUUAUCAAUU | AAUUGAUAAAAUCACCUGG | | [2281-2299] | — |
| 11 | CACUUUAGGAUAAGAAUGU | ACAUUCUUAUCCUAAAGUG | | [2041-2059] | [1701-1719] |
| 12 | GUCACUUUAGGAUAAGAAU | AUUCUUAUCCUAAAGUGAC | | [2039-2057] | [1699-1717] |
| 13 | AGUCACUUUAGGAUAAGAA | UUCUUAUCCUAAAGUGACU | | [2038-2056] | [1698-1716] |
| 14 | AGGUUCAAUUCUACUUCAA | UUGAAGUAGAAUUGAACCU | Rat | [1867-1885] | [1584-1602] |
| 15 | GGCAUCCUUUUACUUUGAC | GUCAAAGUAAAAGGAUGCC | | [1216-1234] | [1080-1098] |
| 16 | GCAGAGUCUUUUGAGAUGU | ACAUCUCAAAAGACUCUGC | | [936-954] | [800-818] |
| 17 | CUCCAGCCUAUCUCAUGAU | AUCAUGAGAUAGGCUGGAG | | [629-647] | [493-511] |
| 18 | CAGUCACUUUAGGAUAAGA | UCUUAUCCUAAAGUGACUG | | [2037-2055] | [1697-1715] |
| 19 | CAAAGUGGGUUUUCUAAAC | GUUUAGAAAACCCACUUUG | | [1595-1613] | — |
| 20 | CGGUCAUUCUUUAUAUCUG | CAGAUAUAAAGAAUGACCG | | [1033-1051] | [897-915] |
| 21 | CGGCAUUGGUGGAAUUGUC | GACAAUUCCACCAAUGCCG | | [869-887] | [733-751] |
| 22 | CCACCUUUUUAUCUUCUAU | AUAGAAGAUAAAAAGGUGG | | [830-848] | [694-712] |
| 23 | GGAGGAGUUAUUUUGAAGU | ACUUCAAAAUAACUCCUCC | | [796-814] | [660-678] |
| 24 | CAGUGGAGUAUGUGACAUU | AAUGUCACAUACUCCACUG | | [697-715] | [561-579] |
| 25 | ACACCUGUUUAACUUUGAC | GUCAAAGUUAAACAGGUGU | | [560-578] | [424-442] |
| 26 | CACACCUGUUUAACUUUGA | UCAAAGUUAAACAGGUGUG | Rb | [559-577] | [423-441] |
| 27 | GCUAUUCACAUCAUUGCAC | GUGCAAUGAUGUGAAUAGC | | [543-561] | [407-425] |
| 28 | GGAUCACAACCUCACCUUC | GAAGGUGAGGUUGUGAUCC | Rat, Ms | [488-506] | [352-370] |
| 29 | UCACUUUUGAUAUGGUCUU | AAGACCAUAUCAAAAGUGA | | [2499-2517] | — |
| 30 | AGUAGGGUGACUUUUUAAA | UUUAAAAAGUCACCCUACU | | [2370-2388] | — |
| 31 | CCCAGGUGAUUUUAUCAAU | AUUGAUAAAAUCACCUGGG | | [2280-2298] | — |
| 32 | GCAUUAAACUUUGACAAGG | CCUUGUCAAAGUUUAAUGC | | [1662-1680] | [1379-1397] |
| 33 | AAGUGGGUUUUCUAAACUA | UAGUUUAGAAAACCCACUU | | [1597-1615] | — |
| 34 | CUCCAUUCAUAUCCGAGCA | UGCUCGGAUAUGAAUGGAG | | [1259-1277] | [1123-1141] |
| 35 | AGAUUUCUUCUCCAUUCAU | AUGAAUGGAGAAGAAAUCU | | [1250-1268] | [1114-1132] |
| 36 | UGGCAUCCUUUUACUUUGA | UCAAAGUAAAAGGAUGCCA | | [1215-1233] | [1079-1097] |
| 37 | UCAAUCUCUCUCCUGGAAU | AUUCCAGGAGAGAGAUUGA | | [1197-1215] | [1061-1079] |
| 38 | CAAGUGUGCAGAGUCUUUU | AAAAGACUCUGCACACUUG | | [929-947] | [793-811] |
| 39 | ACAGUGGAGUAUGUGACAU | AUGUCACAUACUCCACUGU | | [696-714] | [560-578] |
| 40 | CCAGCCUAUCUCAUGAUGA | UCAUCAUGAGAUAGGCUGG | | [631-649] | [495-513] |
| 41 | CUAUUCACAUCAUUGCACA | UGUGCAAUGAUGUGAAUAG | | [544-562] | [408-426] |
| 42 | UACAGCUAUUCACAUCAUU | AAUGAUGUGAAUAGCUGUA | | [539-557] | [403-421] |
| 43 | CACUUUUGAUAUGGUCUUA | UAAGACCAUAUCAAAAGUG | | [2500-2518] | — |
| 44 | CCUGAGUUUUGGUUUGUAA | UUACAAACCAAAACUCAGG | | [2315-2333] | — |
| 45 | AGGAUAAGAAUGUGCCUCU | AGAGGCACAUUCUUAUCCU | | [2047-2065] | [1707-1725] |
| 46 | UGAGCAGUCACUUUAGGAU | AUCCUAAAGUGACUGCUCA | | [2033-2051] | [1693-1711] |
| 47 | GGAGUUUUCUUAUGUGGCC | GGCCACAUAAGAAAACUCC | | [1785-1803] | [1502-1520] |
| 48 | CCACAACCUCAAAACAAAA | UUUUGUUUUGAGGUUGUGG | | [1481-1499] | [1345-1363] |
| 49 | ACCACAACCUCAAAACAAA | UUUGUUUUGAGGUUGUGGU | | [1480-1498] | [1344-1362] |
| 50 | AGAAUCCGGAUUGCUGAAU | AUUCAGCAAUCCGGAUUCU | | [147-165] | [11-29] |
| 51 | CCUUUUACUUUGACCUCUG | CAGAGGUCAAAGUAAAAGG | | [1221-1239] | [1085-1103] |
| 52 | UGAUUACCAAGGUUGUUAU | AUAACAACCUUGGUAAUCA | | [1093-1111] | [957-975] |
| 53 | CCGGUCAUUCUUUAUAUCU | AGAUAUAAAGAAUGACCGG | | [1032-1050] | [896-914] |
| 54 | ACCGGUCAUUCUUUAUAUC | GAUAUAAAGAAUGACCGGU | | [1031-1049] | [895-913] |
| 55 | CGGAGGAGUUAUUUUGAAG | CUUCAAAAUAACUCCUCCG | | [795-813] | [659-677] |
| 56 | CAAUAGCCUUGAUUCUCAU | AUGAGAAUCAAGGCUAUUG | | [751-769] | [615-633] |
| 57 | CUGUUUAACUUUGACUGCU | AGCAGUCAAAGUUAAACAG | | [564-582] | [428-446] |
| 58 | CACCUGUUUAACUUUGACU | AGUCAAAGUUAAACAGGUG | | [561-579] | [425-443] |
| 59 | UGAAUUUUAACAGCACGCU | AGCGUGCUGUUAAAAUUCA | | [385-403] | [249-267] |
| 60 | CCGACAAAUACUACUACAC | GUGUAGUAGUAUUUGUCGG | | [313-331] | [177-195] |
| 61 | CCUUCCUGAAAUAUGAGAA | UUCUCAUAUUUCAGGAAGG | | [292-310] | [156-174] |
| 62 | GCCUUCCUGAAAUAUGAGA | UCUCAUAUUUCAGGAAGGC | | [291-309] | [155-173] |
| 63 | CUGUAAAUCUUUCUGGACA | UGUCCAGAAAGAUUUACAG | | [2201-2219] | — |
| 64 | UGCAUUCAACUUCGUUACU | AGUAACGAAGUUGAAUGCA | | [2099-2117] | — |
| 65 | GCAUUUUGUCUCUUUGUAU | AUACAAAGAGACAAAAUGC | | [1984-2002] | [1644-1662] |
| 66 | GUAGUGGGAGUUUUCUUAU | AUAAGAAAACUCCCACUAC | | [1779-1797] | [1496-1514] |
| 67 | CCAAGUCUGUAGUGGGAGU | ACUCCCACUACAGACUUGG | | [1771-1789] | [1488-1506] |
| 68 | CCGGAUGGGACAGCAAUAU | AUAUUGCUGUCCCAUCCGG | | [1630-1648] | — |
| 69 | AAAGUGGGUUUUCUAAACU | AGUUUAGAAAACCCACUUU | | [1596-1614] | — |
| 70 | ACAAAUUCCAGUGUGCAGA | UCUGCACACUGGAAUUUGU | | [1462-1480] | [1326-1344] |
| 71 | GGUACAAAUUCCAGUGUGC | GCACACUGGAAUUUGUACC | | [1459-1477] | [1323-1341] |
| 72 | CUUCUAUCUUGAAAUCCAU | AUGGAUUUCAAGAUAGAAG | | [1438-1456] | [1302-1320] |
| 73 | GCAUCCUUUUACUUUGACC | GGUCAAAGUAAAAGGAUGC | | [1217-1235] | [1081-1099] |
| 74 | CAGAGUCUUUUGAGAUGUG | CACAUCUCAAAAGACUCUG | | [937-955] | [801-819] |
| 75 | GAGUUAUUUUGAAGUCUUC | GAAGACUUCAAAAUAACUC | | [800-818] | [664-682] |
| 76 | GAGGAGUUAUUUUGAAGUC | GACUUCAAAAUAACUCCUC | | [797-815] | [661-679] |
| 77 | CGACAGUGGAGUAUGUGAC | GUCACAUACUCCACUGUCG | | [694-712] | [558-576] |
| 78 | UCCAGCCUAUCUCAUGAUG | CAUCAUGAGAUAGGCUGGA | | [630-648] | [494-512] |
| 79 | ACCUGUUUAACUUUGACUG | CAGUCAAAGUUAAACAGGU | | [562-580] | [426-444] |
| 80 | UGCACACCUGUUUAACUUU | AAAGUUAAACAGGUGUGCA | | [557-575] | [421-439] |
| 81 | ACAUCAUUGCACACCUGUU | AACAGGUGUGCAAUGAUGU | | [550-568] | [414-432] |
| 82 | GCCUAUAUGAUCUGCCUAC | GUAGGCAGAUCAUAUAGGC | | [519-537] | [383-401] |
| 83 | GGCCGACAAAUACUACUAC | GUAGUAGUAUUUGUCGGCC | | [311-329] | [175-193] |
| 84 | UGAAUGUUUUCCUGUUUGU | ACAAACAGGAAAACAUUCA | | [268-286] | [132-150] |
| 85 | CUGAAUGUUUUCCUGUUUG | CAAACAGGAAAACAUUCAG | | [267-285] | [131-149] |
| 86 | CAGUUUUGUUUCUGGUUGU | ACAACCAGAAACAAAACUG | | [238-256] | [102-120] |
| 87 | UGUUUACUGUCACUUUUGA | UCAAAAGUGACAGUAAACA | | [2490-2508] | — |
| 88 | CAACUUAAGAACUUCUGAA | UUCAGAAGUUCUUAAGUUG | | [2128-2146] | — |
| 89 | CAACUUCGUUACUUGAGCU | AGCUCAAGUAACGAAGUUG | | [2105-2123] | — |
| 90 | UGAUUGCAUUCAACUUCGU | ACGAAGUUGAAUGCAAUCA | | [2095-2113] | — |
| 91 | UCCCUGGUAUUCUUUUUUU | AAAAAAAGAAUACCAGGGA | | [2077-2095] | — |
| 92 | UGCAUUUUGUCUCUUUGUA | UACAAAGAGACAAAAUGCA | | [1983-2001] | [1643-1661] |
| 93 | GUGGGAGUUUUCUUAUGUG | CACAUAAGAAAACUCCCAC | | [1782-1800] | [1499-1517] |
| 94 | AGUCUGUAGUGGGAGUUUU | AAAACUCCCACUACAGACU | | [1774-1792] | [1491-1509] |
| 95 | CAAGUCUGUAGUGGGAGUU | AACUCCCACUACAGACUUG | | [1772-1790] | [1489-1507] |
| 96 | GGACAAUGAGUUUUCUACA | UGUAGAAAACUCAUUGUCC | | [1736-1754] | [1453-1471] |
| 97 | CAGCAAUAUUGUUGGUCAU | AUGACCAACAAUAUUGCUG | | [1640-1658] | — |
| 98 | CACAACCUCAAAACAAAAA | UUUUUGUUUUGAGGUUGUG | | [1482-1500] | [1346-1364] |
| 99 | GACCACAACCUCAAAACAA | UUGUUUUGAGGUUGUGGUC | | [1479-1497] | [1343-1361] |
| 100 | CUAUCUUGAAAUCCAUCUG | CAGAUGGAUUUCAAGAUAG | | [1441-1459] | [1305-1323] |
| 101 | CCCUUUGCUUCUAUCUUGA | UCAAGAUAGAAGCAAAGGG | | [1431-1449] | [1295-1313] |
| 102 | UCCUUUUACUUUGACCUCU | AGAGGUCAAAGUAAAAGGA | | [1220-1238] | [1084-1102] |
| 103 | GUCAUUCUUUAUAUCUGUG | CACAGAUAUAAAGAAUGAC | | [1035-1053] | [899-917] |
| 104 | AGAGCAUGAAUGAGAGUCA | UGACUCUCAUUCAUGCUCU | | [904-922] | [768-786] |
| 105 | GCUUAGGGAUUCACGGCAU | AUGCCGUGAAUCCCUAAGC | | [856-874] | [720-738] |
| 106 | ACCACCUUUUUAUCUUCUA | UAGAAGAUAAAAAGGUGGU | | [829-847] | [693-711] |
| 107 | AGGAGUUAUUUUGAAGUCU | AGACUUCAAAAUAACUCCU | | [798-816] | [662-680] |
| 108 | UGAUUCUCAUGGUAACUUC | GAAGUUACCAUGAGAAUCA | GP, Rb | [760-778] | [624-642] |
| 109 | CUGGAGUGAUCAUGACAAU | AUUGUCAUGAUCACUCCAG | | [736-754] | [600-618] |
| 110 | GGGUAGGUGUGUGUUUUUC | GAAAAACACACACCUACCC | | [67-85] | — |
| 111 | CCUACAUACAGCUAUUCAC | GUGAAUAGCUGUAUGUAGG | | [533-551] | [397-415] |
| 112 | UGAAAUAUGAGAAGGCCGA | UCGGCCUUCUCAUAUUUCA | | [298-316] | [162-180] |
| 113 | GAAGGCGGAAUAAACUUAU | AUAAGUUUAUUCCGCCUUC | | [29-47] | — |
| 114 | AGUUUUGUUUCUGGUUGUU | AACAACCAGAAACAAAACU | | [239-257] | [103-121] |
| 115 | CUGUCACUUUUGAUAUGGU | ACCAUAUCAAAAGUGACAG | | [2496-2514] | — |
| 116 | CCCAUAUAACACCAACAGU | ACUGUUGGUGUUAUAUGGG | | [2466-2484] | — |
| 117 | ACAACUAAUCCCAGGUGAU | AUCACCUGGGAUUAGUUGU | | [2271-2289] | — |
| 118 | CAGAGUUAUACAACUAAUC | GAUUAGUUGUAUAACUCUG | | [2262-2280] | — |
| 119 | GUUCAGAGUUAUACAACUA | UAGUUGUAUAACUCUGAAC | | [2259-2277] | — |
| 120 | GGUUCAGAGUUAUACAACU | AGUUGUAUAACUCUGAACC | | [2258-2276] | — |
| 121 | GUGGUUCAGAGUUAUACAA | UUGUAUAACUCUGAACCAC | | [2256-2274] | — |
| 122 | UGGGAAACUGGGUGGUUAA | UUAACCACCCAGUUUCCCA | | [208-226] | [72-90] |
| 123 | GUAAUUUACUUGGUCUCGU | ACGAGACCAAGUAAAUUAC | | [2008-2026] | [1668-1686] |
| 124 | UCAGUAAUUUACUUGGUCU | AGACCAAGUAAAUUACUGA | | [2005-2023] | [1665-1683] |
| 125 | AGGACGGUAAUCUGCAUUU | AAAUGCAGAUUACCGUCCU | | [1971-1989] | [1631-1649] |
| 126 | AGCAUUAAACUUUGACAAG | CUUGUCAAAGUUUAAUGCU | | [1661-1679] | [1378-1396] |
| 127 | GCCUUUUCCUGGUUCAACA | UGUUGAACCAGGAAAAGGC | | [1536-1554] | — |
| 128 | CCUCAAAACAAAAAAGAUC | GAUCUUUUUUGUUUUGAGG | | [1487-1505] | [1351-1366] |
| | | | | | (16/16 |
| 129 | CAGACCACAACCUCAAAAC | GUUUUGAGGUUGUGGUCUG | | [1477-1495] | [1341-1359] |
| 130 | AGCAGAAGGUUGUGAUUAC | GUAAUCACAACCUUCUGCU | | [1081-1099] | [945-963] |
| 131 | UGCAGAGUCUUUUGAGAUG | CAUCUCAAAAGACUCUGCA | | [935-953] | [799-817] |
| 132 | GGAGAGCAUGAAUGAGAGU | ACUCUCAUUCAUGCUCUCC | | [902-920] | [766-784] |
| 133 | ACAUCUUAAAGGCUCACAG | CUGUGAGCCUUUAAGAUGU | | [86-104] | — |
| 134 | CUAUAUGAUCUGCCUACAU | AUGUAGGCAGAUCAUAUAG | | [521-539] | [385-403] |
| 135 | GAAAAAUCCUUGGGUCAAC | GUUGACCCAAGGAUUUUUC | | [334-352] | [198-216] |
| 136 | AGGCCGACAAAUACUACUA | UAGUAGUAUUUGUCGGCCU | | [310-328] | [174-192] |
| 137 | GGGCUGAAUGUUUUCCUGU | ACAGGAAAACAUUCAGCCC | | [264-282] | [128-146] |
| 138 | UCCCAGGUGAUUUUAUCAA | UUGAUAAAAUCACCUGGGA | | [2279-2297] | — |
| 139 | UGUAAAUCUUUCUGGACAG | CUGUCCAGAAAGAUUUACA | | [2202-2220] | — |
| 140 | CCCUGGUAUUCUUUUUUUG | CAAAAAAAGAAUACCAGGG | | [2078-2096] | — |
| 141 | CUCCCUGGUAUUCUUUUUU | AAAAAAGAAUACCAGGGAG | | [2076-2094] | — |
| 142 | GCCUUGACUCCCUGGUAUU | AAUACCAGGGAGUCAAGGC | | [2069-2087] | — |
| 143 | CAGUAAUUUACUUGGUCUC | GAGACCAAGUAAAUUACUG | | [2006-2024] | [1666-1684] |
| 144 | CUCUUUGUAUCUUCAGUAA | UUACUGAAGAUACAAAGAG | | [1993-2011] | [1653-1671] |
| 145 | CACCUCUUGACAAUGGGAA | UUCCCAUUGUCAAGAGGUG | | [195-213] | [59-77] |
| 146 | AAGGUUCAAUUCUACUUCA | UGAAGUAGAAUUGAACCUU | Rat | [1866-1884] | [1583-1601] |
| 147 | AGUGGGAGUUUUCUUAUGU | ACAUAAGAAAACUCCCACU | | [1781-1799] | [1498-1516] |
| 148 | UGUAGUGGGAGUUUUCUUA | UAAGAAAACUCCCACUACA | | [1778-1796] | [1495-1513] |
| 149 | CUGUAGUGGGAGUUUUCUU | AAGAAAACUCCCACUACAG | | [1777-1795] | [1494-1512] |
| 150 | UGGUCAUGCAGCAUUAAAC | GUUUAAUGCUGCAUGACCA | GP, Rat, | [1652-1670] | [1369-1387] |
| | | | Ms | | |
| 151 | GGUUUUCUAAACUACCGUC | GACGGUAGUUUAGAAAACC | | [1602-1620] | — |
| 152 | UGGAGGAAUUAGGCAAAGU | ACUUUGCCUAAUUCCUCCA | | [1582-1600] | — |
| 153 | GGAUUGCUGAAUCUUCCCU | AGGGAAGAUUCAGCAAUCC | | [154-172] | [18-36] |
| 154 | CUUUGCUUCUAUCUUGAAA | UUUCAAGAUAGAAGCAAAG | | [1433-1451] | [1297-1315] |
| 155 | CCUUUGCUUCUAUCUUGAA | UUCAAGAUAGAAGCAAAGG | | [1432-1450] | [1296-1314] |
| 156 | UGCACCGGUCAUUCUUUAU | AUAAAGAAUGACCGGUGCA | | [1028-1046] | [892-910] |
| 157 | GUGCAGAGUCUUUUGAGAU | AUCUCAAAAGACUCUGCAC | | [934-952] | [798-816] |
| 158 | AGGAGAGCAUGAAUGAGAG | CUCUCAUUCAUGCUCUCCU | | [901-919] | [765-783] |
| 159 | CUCACCACCUUUUUAUCUU | AAGAUAAAAAGGUGGUGAG | | [826-844] | [690-708] |
| 160 | GUAUACUCACCACCUUUUU | AAAAAGGUGGUGAGUAUAC | | [821-839] | [685-703] |
| 161 | CACAUCUUAAAGGCUCACA | UGUGAGCCUUUAAGAUGUG | | [85-103] | — |
| 162 | UCACAUCUUAAAGGCUCAC | GUGAGCCUUUAAGAUGUGA | | [84-102] | — |
| 163 | GUGGAGUAUGUGACAUUCA | UGAAUGUCACAUACUCCAC | | [699-717] | [563-581] |
| 164 | CCAUUCUCUCCAGCCUAUC | GAUAGGCUGGAGAGAAUGG | | [622-640] | [486-504] |
| 165 | CCUAUAUGAUCUGCCUACA | UGUAGGCAGAUCAUAUAGG | | [520-538] | [384-402] |
| 166 | UCCUGUGUGUCGCAAUCUG | CAGAUUGCGACACACAGGA | | [413-431] | [277-295] |
| 167 | AGAAAAAUCCUUGGGUCAA | UUGACCCAAGGAUUUUUCU | | [333-351] | [197-215] |
| 168 | AGGGCUGAAUGUUUUCCUG | CAGGAAAACAUUCAGCCCU | | [263-281] | [127-145] |
| 169 | AAACAAAAUUACCGAGAAC | GUUCUCGGUAAUUUUGUUU | | [2441-2459] | — |
| 170 | UGGUUUGUAAUCUUUUGUC | GACAAAAGAUUACAAACCA | | [2324-2342] | — |
| 171 | GAGUUUUGGUUUGUAAUCU | AGAUUACAAACCAAAACUC | | [2318-2336] | — |
| 172 | CAGGUGAUUUUAUCAAUUC | GAAUUGAUAAAAUCACCUG | | [2282-2300] | — |
| 173 | UGAAGUUCUUAAAGCCCAU | AUGGGCUUUAAGAACUUCA | | [2160-2178] | — |
| 174 | GGGAAACUGGGUGGUUAAC | GUUAACCACCCAGUUUCCC | | [209-227] | [73-91] |
| 175 | GCAACUUAAGAACUUCUGA | UCAGAAGUUCUUAAGUUGC | | [2127-2145] | — |
| 176 | AACUUCGUUACUUGAGCUU | AAGCUCAAGUAACGAAGUU | | [2106-2124] | — |
| 177 | CUCUUGACAAUGGGAAACU | AGUUUCCCAUUGUCAAGAG | | [198-216] | [62-80] |
| 178 | ACGGUAAUCUGCAUUUUGU | ACAAAAUGCAGAUUACCGU | | [1974-1992] | [1634-1652] |
| 179 | CUCGGACUUUGGCAAAGAG | CUCUUUGCCAAAGUCCGAG | | [1804-1822] | [1521-1539] |
| 180 | CAAUAUUGUUGGUCAUGCA | UGCAUGACCAACAAUAUUG | | [1643-1661] | [1367-1378] |
| | | | | | (12/12) |
| 181 | GCAAUAUUGUUGGUCAUGC | GCAUGACCAACAAUAUUGC | | [1642-1660] | [1367-1377] |
| | | | | | (11/11) |
| 182 | ACAGCAAUAUUGUUGGUCA | UGACCAACAAUAUUGCUGU | | [1639-1657] | — |
| 183 | GAAUUAGGCAAAGUGGGUU | AACCCACUUUGCCUAAUUC | | [1587-1605] | — |
| 184 | AGAUGGAGGAAUUAGGCAA | UUGCCUAAUUCCUCCAUCU | Ms | [1579-1597] | — |
| 185 | ACAGGAGAUGGAGGAAUUA | UAAUUCCUCCAUCUCCUGU | | [1574-1592] | — |
| 186 | CAACCUCAAAACAAAAAAG | CUUUUUUGUUUUGAGGUUG | | [1484-1502] | [1348-1366] |
| 187 | CCAGGAUUGAAGUGGAUGG | CCAUCCACUUCAAUCCUGG | | [1336-1354] | [1200-1218] |
| 188 | GACAGAAAAUCUCAUAAGG | CCUUAUGAGAUUUUCUGUC | Rat | [1289-1307] | [1153-1171] |
| 189 | GAAUGGCAUCCUUUUACUU | AAGUAAAAGGAUGCCAUUC | | [1212-1230] | [1076-1094] |
| 190 | GUUUUGGAAUUGCAGAUGA | UCAUCUGCAAUUCCAAAAC | Rat, Ms | [1125-1143] | [989-1007] |
| 191 | AAGUUUUGGAAUUGCAGAU | AUCUGCAAUUCCAAAACUU | Rat | [1123-1141] | [987-1005] |
| 192 | GUCAAACAGAGGAGAGCAU | AUGCUCUCCUCUGUUUGAC | | [892-910] | [756-774] |
| 193 | CUUAGGGAUUCACGGCAUU | AAUGCCGUGAAUCCCUAAG | | [857-875] | [721-739] |
| 194 | GAAGUCUUCUGGUAUACUC | GAGUAUACCAGAAGACUUC | | [810-828] | [674-692] |
| 195 | UUUGAAGUCUUCUGGUAUA | UAUACCAGAAGACUUCAAA | | [807-825] | [671-689] |
| 196 | UUGAUUCUCAUGGUAACUU | AAGUUACCAUGAGAAUCAA | GP, Rb | [759-777] | [623-641] |
| 197 | UCCCUUGCCUCCAUUCUCU | AGAGAAUGGAGGCAAGGGA | | [612-630] | [476-494] |
| 198 | GAGAAAGCAAUUGGAUCAC | GUGAUCCAAUUGCUUUCUC | | [476-494] | [340-358] |
| 199 | AAGGCGGAAUAAACUUAUU | AAUAAGUUUAUUCCGCCUU | | [30-48] | — |
| 200 | CUGAGUUUUGGUUUGUAAU | AUUACAAACCAAAACUCAG | | [2316-2334] | — |
| 201 | GGUGAUUUUAUCAAUUCCA | UGGAAUUGAUAAAAUCACC | | [2284-2302] | — |
| 202 | CUGAAGUUCUUAAAGUUCU | AGAACUUUAAGAACUUCAG | | [2142-2160] | — |
| 203 | AUCUGCAUUUUGUCUCUUU | AAAGAGACAAAAUGCAGAU | | [1980-1998] | [1640-1658] |
| 204 | GAAACAGAAAACCUCCUUU | AAAGGAGGUUUUCUGUUUC | Rat | [1703-1721] | [1420-1438] |
| 205 | UGUUGGUCAUGCAGCAUUA | UAAUGCUGCAUGACCAACA | | [1649-1667] | [1367-1384] |
| | | | | | (18/18) |
| 206 | GCUGAAUCUUCCCUGUUGC | GCAACAGGGAAGAUUCAGC | | [159-177] | [23-41] |
| 207 | ACAACCUGUUGACUUCCCU | AGGGAAGUCAACAGGUUGU | | [1552-1570] | — |
| 208 | CCUUUUCCUGGUUCAACAA | UUGUUGAACCAGGAAAAGG | | [1537-1555] | — |
| 209 | GGUGCCUUUUCCUGGUUCA | UGAACCAGGAAAAGGCACC | GP | [1533-1551] | — |
| 210 | UGCAGACCACAACCUCAAA | UUUGAGGUUGUGGUCUGCA | GP | [1475-1493] | [1339-1357] |
| 211 | GGCUUUCGAACAACAAUAU | AUAUUGUUGUUCGAAAGCC | | [1307-1325] | [1171-1189] |
| 212 | GGGACUGGACAGAAAAUCU | AGAUUUUCUGUCCAGUCCC | Rat | [1282-1300] | [1146-1164] |
| 213 | GGACAAAUGUUCCAUUCCU | AGGAAUGGAACAUUUGUCC | | [115-133] | — |
| 214 | CACCACCUUUUUAUCUUCU | AGAAGAUAAAAAGGUGGUG | | [828-846] | [692-710] |
| 215 | UCACCACCUUUUUAUCUUC | GAAGAUAAAAAGGUGGUGA | | [827-845] | [691-709] |
| 216 | AUACUCACCACCUUUUUAU | AUAAAAAGGUGGUGAGUAU | | [823-841] | [687-705] |
| 217 | CUCCAUUCUCUCCAGCCUA | UAGGCUGGAGAGAAUGGAG | | [620-638] | [484-502] |
| 218 | CACUGAGAAAGCAAUUGGA | UCCAAUUGCUUUCUCAGUG | | [472-490] | [336-354] |
| 219 | UGGUUAGGGCUGAAUGUUU | AAACAUUCAGCCCUAACCA | | [258-276] | [122-140] |
| 220 | GGUUUGUAAUCUUUUGUCC | GGACAAAAGAUUACAAACC | | [2325-2343] | — |
| 221 | UGAGUUUUGGUUUGUAAUC | GAUUACAAACCAAAACUCA | | [2317-2335] | — |
| 222 | UCAAUUCCAGUGUUACCAU | AUGGUAACACUGGAAUUGA | | [2294-2312] | [1329-1339] |
| | | | | | (11/11) |
| 223 | CUUCUGAAGUUCUUAAAGU | ACUUUAAGAACUUCAGAAG | | [2139-2157] | — |
| 224 | AGCAACUUAAGAACUUCUG | CAGAAGUUCUUAAGUUGCU | | [2126-2144] | — |
| 225 | CUUCGUUACUUGAGCUUCA | UGAAGCUCAAGUAACGAAG | | [2108-2126] | — |
| 226 | CUUCAGUAAUUUACUUGGU | ACCAAGUAAAUUACUGAAG | | [2003-2021] | [1663-1681] |
| 227 | CAGUAAUUGAGUUAUAGGA | UCCUAUAACUCAAUUACUG | | [1949-1967] | [1615-1627] |
| | | | | | (13/13) |
| 228 | CCUCUUGACAAUGGGAAAC | GUUUCCCAUUGUCAAGAGG | | [197-215] | [61-79 |
| 229 | CGGUAAUCUGCAUUUUGUC | GACAAAAUGCAGAUUACCG | | [1975-1993] | [1635-1653] |
| 230 | AUAGGAAUAAGGACGGUAA | UUACCGUCCUUAUUCCUAU | | [1962-1980] | [1622-1640] |
| 231 | UGAGUUAUAGGAAUAAGGA | UCCUUAUUCCUAUAACUCA | | [1956-1974] | [1616-1634]+ |
| 232 | GGAAUUAGGCAAAGUGGGU | ACCCACUUUGCCUAAUUCC | | [1586-1604] | — |
| 233 | CAGGAUUGAAGUGGAUGGU | ACCAUCCACUUCAAUCCUG | | [1337-1355] | [1201-1219] |
| 234 | GGGCUUUCGAACAACAAUA | UAUUGUUGUUCGAAAGCCC | | [1306-1324] | [1170-1188] |
| 235 | CAGAUGAACAAGCGUGGCU | AGCCACGCUUGUUCAUCUG | | [1137-1155] | [1001-1019] |
| 236 | ACAGAGGAGAGCAUGAAUG | CAUUCAUGCUCUCCUCUGU | | [897-915] | [761-779] |
| 237 | UCUUCUAUAUCCUUGGCUU | AAGCCAAGGAUAUAGAAGA | | [841-859] | [705-723] |
| 238 | UGGAGUGAUCAUGACAAUA | UAUUGUCAUGAUCACUCCA | | [737-755] | [601-619] |
| 239 | ACUGGAGUGAUCAUGACAA | UUGUCAUGAUCACUCCAGU | | [735-753] | [599-617] |
| 240 | CACACUGAGAAAGCAAUUG | CAAUUGCUUUCUCAGUGUG | | [470-488] | [334-352] |
| 241 | GCUUCCUGUGUGUCGCAAU | AUUGCGACACACAGGAAGC | | [410-428] | [274-292] |
| 242 | UGUUUGGUUAGGGCUGAAU | AUUCAGCCCUAACCAAACA | | [254-272] | [118-136] |
| 243 | GUGUUACCAUCUCCUGAGU | ACUCAGGAGAUGGUAACAC | | [2303-2321] | — |
| 244 | CAAUUCCAGUGUUACCAUC | GAUGGUAACACUGGAAUUG | | [2295-2313] | [1329-1339] |
| | | | | | (11/11 |
| 245 | GUGAUUUUAUCAAUUCCAG | CUGGAAUUGAUAAAAUCAC | | [2285-2303] | — |
| 246 | CAGCAACUUAAGAACUUCU | AGAAGUUCUUAAGUUGCUG | | [2125-2143] | — |
| 247 | ACUCCCUGGUAUUCUUUUU | AAAAAGAAUACCAGGGAGU | | [2075-2093] | — |
| 248 | AGGUUUGAGCAGUCACUUU | AAAGUGACUGCUCAAACCU | | [2028-2046] | [1688-1706] |
| 249 | CUGCAUUUUGUCUCUUUGU | ACAAAGAGACAAAAUGCAG | | [1982-2000] | [1642-1660] |
| 250 | GUAAUCUGCAUUUUGUCUC | GAGACAAAAUGCAGAUUAC | | [1977-1995] | [1637-1655] |
| 251 | ACCUCUUGACAAUGGGAAA | UUUCCCAUUGUCAAGAGGU | | [196-214] | [60-78] |
| 252 | UUGUUGGUCAUGCAGCAUU | AAUGCUGCAUGACCAACAA | | [1648-1666] | [1367-1383] |
| | | | | | (17/17 |
| 253 | GGCAAAGUGGGUUUUCUAA | UUAGAAAACCCACUUUGCC | | [1593-1611] | — |
| 254 | GUGCCUUUUCCUGGUUCAA | UUGAACCAGGAAAAGGCAC | GP | [1534-1552] | — |
| 255 | CCUGAGUCUUGGAAGUGGA | UCCACUUCCAAGACUCAGG | GP, Rat, Ms | [1005-1023] | [869-887] |
| 256 | CUACUGAGUUCAUCCGGAG | CUCCGGAUGAACUCAGUAG | | [781-799] | [645-663] |
| 257 | GCUACUGAGUUCAUCCGGA | UCCGGAUGAACUCAGUAGC | | [780-798] | [644-662] |
| 258 | CCUAUCUCAUGAUGAGAAA | UUUCUCAUCAUGAGAUAGG | | [635-653] | [499-517] |
| 259 | UGGUGGCCUAUAUGAUCUG | CAGAUCAUAUAGGCCACCA | | [514-532] | [378-396] |
| 260 | CUGGUGGCCUAUAUGAUCU | AGAUCAUAUAGGCCACCAG | | [513-531] | [377-395] |
| 261 | ACAACCUCACCUUCCACAA | UUGUGGAAGGUGAGGUUGU | | [493-511] | [357-375] |
| 262 | UUGUUUGGUUAGGGCUGAA | UUCAGCCCUAACCAAACAA | | [253-271] | [117-135] |
| 263 | AGAAGGAAGGCGGAAUAAA | UUUAUUCCGCCUUCCUUCU | | [24-42] | — |
| 264 | UGGUGGUUCAGAGUUAUAC | GUAUAACUCUGAACCACCA | | [2254-2272] | — |
| 265 | GGUUUGGUGGUUCAGAGUU | AACUCUGAACCACCAAACC | | [2250-2268] | — |
| 266 | GAGGUUUGGUGGUUCAGAG | CUCUGAACCACCAAACCUC | | [2248-2266] | — |
| 267 | CAGAGAAGGAAGGCGGAAU | AUUCCGCCUUCCUUCUCUG | | [21-39] | — |
| 268 | AGCCCAUGGAUCCUUUCUC | GAGAAAGGAUCCAUGGGCU | | [2172-2190] | — |
| 269 | GAAGUUCUUAAAGCCCAUG | CAUGGGCUUUAAGAACUUC | | [2161-2179] | — |
| 270 | UCAGCAACUUAAGAACUUC | GAAGUUCUUAAGUUGCUGA | | [2124-2142] | — |
| 271 | UGACUCCCUGGUAUUCUUU | AAAGAAUACCAGGGAGUCA | | [2073-2091] | — |
| 272 | UUGACUCCCUGGUAUUCUU | AAGAAUACCAGGGAGUCAA | | [2072-2090] | — |
| 273 | UCAGGUUUGAGCAGUCACU | AGUGACUGCUCAAACCUGA | | [2026-2044] | [1686-1704] |
| 274 | CGUCAGGUUUGAGCAGUCA | UGACUGCUCAAACCUGACG | | [2024-2042] | [1684-1702] |
| 275 | UCGUCAGGUUUGAGCAGUC | GACUGCUCAAACCUGACGA | | [2023-2041] | [1683-1701] |
| 276 | CUCGUCAGGUUUGAGCAGU | ACUGCUCAAACCUGACGAG | | [2022-2040] | [1682-1700] |
| 277 | UACUUGGUCUCGUCAGGUU | AACCUGACGAGACCAAGUA | | [2014-2032] | [1674-1692] |
| 278 | UCUCUUUGUAUCUUCAGUA | UACUGAAGAUACAAAGAGA | | [1992-2010] | [1652-1670] |
| 279 | UCUGCAUUUUGUCUCUUUG | CAAAGAGACAAAAUGCAGA | | [1981-1999] | [1641-1659] |
| 280 | GACGGUAAUCUGCAUUUUG | CAAAAUGCAGAUUACCGUC | | [1973-1991] | [1633-1651] |
| 281 | GGGACAGCAAUAUUGUUGG | CCAACAAUAUUGCUGUCCC | | [1636-1654] | — |
| 282 | GAAAAUCUCAUAAGGGCUU | AAGCCCUUAUGAGAUUUUC | | [1293-1311] | [1157-1175] |
| 283 | UUGUUAUGCACCCAUCCAA | UUGGAUGGGUGCAUAACAA | | [1105-1123] | [969-987] |
| 284 | CCAGCAGAAGGUUGUGAUU | AAUCACAACCUUCUGCUGG | | [1079-1097] | [943-961] |
| 285 | CUGAGUCUUGGAAGUGGAU | AUCCACUUCCAAGACUCAG | GP, Rat, Ms | [1006-1024] | [870-888] |
| 286 | CUGCUCUCUGCUUGAAUUU | AAAUUCAAGCAGAGAGCAG | | [373-391] | [237-255] |
| 287 | GCGGAAUAAACUUAUUCAU | AUGAAUAAGUUUAUUCCGC | | [33-51] | — |
| 288 | AGCAGAGGUUUGGUGGUUC | GAACCACCAAACCUCUGCU | | [2244-2262] | — |
| 289 | UGAUAGCAGAGGUUUGGUG | CACCAAACCUCUGCUAUCA | | [2240-2258] | — |
| 290 | CCCAUGGAUCCUUUCUCAG | CUGAGAAAGGAUCCAUGGG | | [2174-2192] | — |
| 291 | CUGAAGUUCUUAAAGCCCA | UGGGCUUUAAGAACUUCAG | | [2159-2177] | — |
| 292 | UUCAGCAACUUAAGAACUU | AAGUUCUUAAGUUGCUGAA | | [2123-2141] | — |
| 293 | GACUCCCUGGUAUUCUUUU | AAAAGAAUACCAGGGAGUC | | [2074-2092] | — |
| 294 | CAGGUUUGAGCAGUCACUU | AAGUGACUGCUCAAACCUG | | [2027-2045] | [1687-1705] |
| 295 | GUCUCUUUGUAUCUUCAGU | ACUGAAGAUACAAAGAGAC | | [1991-2009] | [1651-1669] |
| 296 | GACAGCAAUAUUGUUGGUC | GACCAACAAUAUUGCUGUC | | [1638-1656] | — |
| 297 | UGGGACAGCAAUAUUGUUG | CAACAAUAUUGCUGUCCCA | | [1635-1653] | — |
| 298 | AGAUGUGGGAUGAUCGUGA | UCACGAUCAUCCCACAUCU | | [949-967] | [813-831] |
| 299 | GCAAUUGGAUCACAACCUC | GAGGUUGUGAUCCAAUUGC | | [482-500] | [346-364] |
| 300 | CUACUACACAAGAAAAAUC | GAUUUUUCUUGUGUAGUAG | | [323-341] | [187-205] |
| 301 | UCCAGUGUGAACAGCAAUU | AAUUGCUGUUCACACUGGA | | [2519-2537] | — |
| 302 | GGUUUUCAGUUUUGUUUCU | AGAAACAAAACUGAAAACC | | [232-250] | [96-114] |
| 303 | CUAAGUAGGGUGACUUUUU | AAAAAGUCACCCUACUUAG | | [2367-2385] | — |
| 304 | UCCAGUGUUACCAUCUCCU | AGGAGAUGGUAACACUGGA | | [2299-2317] | — |
| 305 | CCUGGUAUUCUUUUUUUGA | UCAAAAAAAGAAUACCAGG | | [2079-2097] | — |
| 306 | GCAGCAUUAAACUUUGACA | UGUCAAAGUUUAAUGCUGC | GP, Rat, Ms | [1659-1677] | [1376-1394] |
| 307 | GCUGGACAAAUGUUCCAUU | AAUGGAACAUUUGUCCAGC | | [112-130] | — |
| 308 | UGAUCCUGCUUCCUGUGUG | CACACAGGAAGCAGGAUCA | | [403-421] | [267-285] |
| 309 | CCACAGAAGAUUUCCUAAG | CUUAGGAAAUCUUCUGUGG | | [2353-2371] | — |
| 310 | GGGUGGUUAACCACUGGUU | AACCAGUGGUUAACCACCC | | [217-235] | [81-99] |
| 311 | GCUUCAGCAACUUAAGAAC | GUUCUUAAGUUGCUGAAGC | | [2121-2139] | — |
| 312 | GAGUUUUCUACAAUAGCUA | UAGCUAUUGUAGAAAACUC | | [1743-1761] | [1460-1478] |
| 313 | UGAGUUUUCUACAAUAGCU | AGCUAUUGUAGAAAACUCA | | [1742-1760] | [1459-1477] |
| 314 | GGGACAAUGAGUUUUCUAC | GUAGAAAACUCAUUGUCCC | | [1735-1753] | [1452-1470] |
| 315 | AGGCAAAGUGGGUUUUCUA | UAGAAAACCCACUUUGCCU | | [1592-1610] | — |
| 316 | GAACAGGAGAUGGAGGAAU | AUUCCUCCAUCUCCUGUUC | | [1572-1590] | — |
| 317 | CCAUUCCUGAAGGACCUCU | AGAGGUCCUUCAGGAAUGG | | [126-144] | — |
| 318 | GACAAAUGUUCCAUUCCUG | CAGGAAUGGAACAUUUGUC | | [116-134] | — |
| 319 | GCCUAUCUCAUGAUGAGAA | UUCUCAUCAUGAGAUAGGC | | [634-652] | [498-516] |
| 320 | GUCUGCUCUCUGCUUGAAU | AUUCAAGCAGAGAGCAGAC | | [371-389] | [235-253] |
| 321 | CCACUGGUUUUCAGUUUUG | CAAAACUGAAAACCAGUGG | | [227-245] | [91-109] |
| 322 | ACUGUAGCAAGGCUUGAUA | UAUCAAGCCUUGCUACAGU | | [2226-2244] | — |
| 323 | UCAUGCAGCAUUAAACUUU | AAAGUUUAAUGCUGCAUGA | GP, Rat, Ms | [1655-1673] | [1372-1390] |
| 324 | AAAAUCUCAUAAGGGCUUU | AAAGCCCUUAUGAGAUUUU | | [1294-1312] | [1158-1176] |
| 325 | CCAUCCAAAGUUUUGGAAU | AUUCCAAAACUUUGGAUGG | | [1116-1134] | [980-998] |
| 326 | CCCUAAGUUUGAAGGGCAU | AUGCCCUUCAAACUUAGGG | | [983-1001] | [847-865] |
| 327 | AGCCUUGAUUCUCAUGGUA | UACCAUGAGAAUCAAGGCU | GP, Rb | [755-773] | [619-637] |
| 328 | CACCAACAGUGUACAUGUU | AACAUGUACACUGUUGGUG | | [2475-2493] | — |
| 329 | CGGAUGGGACAGCAAUAUU | AAUAUUGCUGUCCCAUCCG | | [1631-1649] | — |
| 330 | GGAACAGGAGAUGGAGGAA | UUCCUCCAUCUCCUGUUCC | | [1571-1589] | — |
| 331 | CCAUCUGGUACAAAUUCCA | UGGAAUUUGUACCAGAUGG | | [1453-1471] | [1317-1335] |
| 332 | CUAUCUCAUGAUGAGAAAA | UUUUCUCAUCAUGAGAUAG | | [636-654] | [500-518] |
| 333 | CUGCCUACAUACAGCUAUU | AAUAGCUGUAUGUAGGCAG | | [530-548] | [394-412] |
| 334 | UGGAUCACAACCUCACCUU | AAGGUGAGGUUGUGAUCCA | Rat, Ms | [487-505] | [351-369] |
| 335 | UGCUCUCUGCUUGAAUUUU | AAAAUUCAAGCAGAGAGCA | | [374-392] | [238-256] |
| 336 | UUCCUAAGUAGGGUGACUU | AAGUCACCCUACUUAGGAA | | [2364-2382] | — |
| 337 | ACCACUGGUUUUCAGUUUU | AAAACUGAAAACCAGUGGU | | [226-244] | [90-108] |
| 338 | GGAUCCUUUCUCAGAAAAA | UUUUUCUGAGAAAGGAUCC | | [2179-2197] | — |
| 339 | UGGGACAAUGAGUUUUCUA | UAGAAAACUCAUUGUCCCA | Rat, Ms | [1734-1752] | [1451-1469] |
| 340 | GUGGGACAAUGAGUUUUCU | AGAAAACUCAUUGUCCCAC | Rat, Ms | [1733-1751] | [1450-1468] |
| 341 | CCUGGUUCAACAACCUGUU | AACAGGUUGUUGAACCAGG | | [1543-1561] | — |
| 342 | CCUCUCCAGAAUCCGGAUU | AAUCCGGAUUCUGGAGAGG | | [140-158] | [4-22] |
| 343 | CAUCCAAAGUUUUGGAAUU | AAUUCCAAAACUUUGGAUG | | [1117-1135] | [981-999] |
| 344 | CUGGUAUACUCACCACCUU | AAGGUGGUGAGUAUACCAG | | [818-836] | [682-700] |
| 345 | UAAGUAGGGUGACUUUUUA | UAAAAAGUCACCCUACUUA | | [2368-2386] | — |
| 346 | GGAUAAGAAUGUGCCUCUC | GAGAGGCACAUUCUUAUCC | | [2048-2066] | [1708-1726] |
| 347 | CCAGUCUGGAUCCUAGAAA | UUUCUAGGAUCCAGACUGG | | [1849-1867] | [1566-1584] |
| 348 | CGAUAUUCCAGUCUGGAUC | GAUCCAGACUGGAAUAUCG | | [1842-1860] | [1559-1577] |
| 349 | GAGACCAAUGUGGGACAAU | AUUGUCCCACAUUGGUCUC | Rat, Ms | [1724-1742] | [1441-1459] |
| 350 | CAGCAUUAAACUUUGACAA | UUGUCAAAGUUUAAUGCUG | GP | [1660-1678] | [1377-1395] |
| 351 | GGAAGAUUUCUUCUCCAUU | AAUGGAGAAGAAAUCUUCC | | [1247-1265] | [1111-1129] |
| 352 | AGGAAGAUUUCUUCUCCAU | AUGGAGAAGAAAUCUUCCU | | [1246-1264] | [1110-1128] |
| 353 | CCAAAGUUUUGGAAUUGCA | UGCAAUUCCAAAACUUUGG | | [1120-1138] | [984-1002] |
| 354 | CAAAUGUUCCAUUCCUGAA | UUCAGGAAUGGAACAUUUG | | [118-136] | — |
| 355 | CGCUGGACAAAUGUUCCAU | AUGGAACAUUUGUCCAGCG | | [111-129] | — |
| 356 | UGUGUGUUUUUCACAUCUU | AAGAUGUGAAAAACACACA | | [74-92] | — |
| 357 | GGAUGCCUUCCUGAAAUAU | AUAUUUCAGGAAGGCAUCC | | [287-305] | [151-169] |
| 358 | UGUUUCUGGUUGUUUGGUU | AACCAAACAACCAGAAACA | | [244-262] | [108-126] |
| 359 | CCAACAGUGUACAUGUUUA | UAAACAUGUACACUGUUGG | | [2477-2495] | — |
| 360 | UCCUAAGUAGGGUGACUUU | AAAGUCACCCUACUUAGGA | | [2365-2383] | — |
| 361 | UGGAUCCUUUCUCAGAAAA | UUUUCUGAGAAAGGAUCCA | | [2178-2196] | — |
| 362 | UGAAGUUCUUAAAGUUCUG | CAGAACUUUAAGAACUUCA | | [2143-2161] | — |
| 363 | UCACCGAUAUUCCAGUCUG | CAGACUGGAAUAUCGGUGA | | [1838-1856] | [1555-1573] |
| 364 | UGCAGCAUUAAACUUUGAC | GUCAAAGUUUAAUGCUGCA | GP, Rat, Ms | [1658-1676] | [1375-1393] |
| 365 | UGGUACAAAUUCCAGUGUG | CACACUGGAAUUUGUACCA | | [1458-1476] | [1322-1340] |
| 366 | CUGGUACAAAUUCCAGUGU | ACACUGGAAUUUGUACCAG | | [1457-1475] | [1321-1339] |
| 367 | CGAACAACAAUAUUCACCA | UGGUGAAUAUUGUUGUUCG | | [1313-1331] | [1177-1195] |
| 368 | ACAGUUCUGUCCAGAGAAG | CUUCUCUGGACAGAACUGU | | [10-28] | — |
| 369 | CCCAUCCAAAGUUUUGGAA | UUCCAAAACUUUGGAUGGG | | [1115-1133] | [979-997] |
| 370 | GCCCUAAGUUUGAAGGGCA | UGCCCUUCAAACUUAGGGC | | [982-1000] | [846-864] |
| 371 | CGAAACACGACAGUGGAGU | ACUCCACUGUCGUGUUUCG | | [687-705] | [551-569] |
| 372 | GGCUAAAUCCCAUCCAGUC | GACUGGAUGGGAUUUAGCC | Rat, Ms | [667-685] | [531-549] |
| 373 | GUGUGUGUUUUUCACAUCU | AGAUGUGAAAAACACACAC | | [73-91] | — |
| 374 | GGUGUGUGUUUUUCACAUC | GAUGUGAAAAACACACACC | | [72-90] | — |
| 375 | UCACAUCAUUGCACACCUG | CAGGUGUGCAAUGAUGUGA | | [548-566] | [412-430] |
| 376 | UCAUUUUGCAGCCGCACAC | GUGUGCGGCUGCAAAAUGA | | [456-474] | [320-338] |
| 377 | GGCACCUGCUCAUUUUGCA | UGCAAAAUGAGCAGGUGCC | GP, Rat, Ms | [447-465] | [311-329] |
| 378 | CCUAAGUAGGGUGACUUUU | AAAAGUCACCCUACUUAGG | | [2366-2384] | — |
| 379 | AACCACUGGUUUUCAGUUU | AAACUGAAAACCAGUGGUU | | [225-243] | [89-107] |
| 380 | AGGCUUGAUAGCAGAGGUU | AACCUCUGCUAUCAAGCCU | | [2235-2253] | — |
| 381 | CUAGAAAGGUUCAAUUCUA | UAGAAUUGAACCUUUCUAG | | [1861-1879] | [1578-1596] |
| 382 | ACAGGUCUGAAACAGAAAA | UUUUCUGUUUCAGACCUGU | GP, Rat | [1695-1713] | [1412-1430] |
| 383 | UUCUAAACUACCGUCUCUU | AAGAGACGGUAGUUUAGAA | Ms | [1606-1624] | — |
| 384 | GGGUUUUCUAAACUACCGU | ACGGUAGUUUAGAAAACCC | | [1601-1619] | — |
| 385 | CCUGUUGACUUCCCUGGAA | UUCCAGGGAAGUCAACAGG | | [1556-1574] | — |
| 386 | UGAGGAUGUUUUCCAGUAU | AUACUGGAAAACAUCCUCA | Ms | [1373-1391] | [1237-1255] |
| 387 | CUCUCCAGAAUCCGGAUUG | CAAUCCGGAUUCUGGAGAG | | [141-159] | [5-23] |
| 388 | GCUUUCGAACAACAAUAUU | AAUAUUGUUGUUCGAAAGC | | [1308-1326] | [1172-1190] |
| 389 | UGGACAGAAAAUCUCAUAA | UUAUGAGAUUUUCUGUCCA | Rat | [1287-1305] | [1151-1169] |
| 390 | CUGGACAGAAAAUCUCAUA | UAUGAGAUUUUCUGUCCAG | Rat | [1286-1304] | [1150-1168] |
| 391 | UCCAUUCAUAUCCGAGCAG | CUGCUCGGAUAUGAAUGGA | | [1260-1278] | [1124-1142] |
| 392 | CUCCUGGAAUGGCAUCCUU | AAGGAUGCCAUUCCAGGAG | | [1206-1224] | [1070-1088] |
| 393 | GCACAGUUCUGUCCAGAGA | UCUCUGGACAGAACUGUGC | | [8-26] | — |
| 394 | GCGCUGGACAAAUGUUCCA | UGGAACAUUUGUCCAGCGC | | [110-128] | — |
| 395 | UCAUCCGGAGGAGUUAUUU | AAAUAACUCCUCCGGAUGA | | [790-808] | [654-672] |
| 396 | AAACACGACAGUGGAGUAU | AUACUCCACUGUCGUGUUU | | [689-707] | [553-571] |
| 397 | ACCUCACCUUCCACAAGCU | AGCUUGUGGAAGGUGAGGU | | [496-514] | [360-378] |
| 398 | CUACACAAGAAAAAUCCUU | AAGGAUUUUUCUUGUGUAG | | [326-344] | [190-208] |
| 399 | AGAUUUCCUAAGUAGGGUG | CACCCUACUUAGGAAAUCU | | [2360-2378] | — |
| 400 | CUCCUGAGUUUUGGUUUGU | ACAAACCAAAACUCAGGAG | | [2313-2331] | — |
| 401 | UGGGUGGUUAACCACUGGU | ACCAGUGGUUAACCACCCA | | [216-234] | [80-98] |
| 402 | GAUCCUUUCUCAGAAAAAU | AUUUUUCUGAGAAAGGAUC | | [2180-2198] | — |
| 403 | GAAGUUCUUAAAGUUCUGA | UCAGAACUUUAAGAACUUC | | [2144-2162] | — |
| 404 | GGAUCCUAGAAAGGUUCAA | UUGAACCUUUCUAGGAUCC | | [1856-1874] | [1573-1591] |
| 405 | UCUGGAUCCUAGAAAGGUU | AACCUUUCUAGGAUCCAGA | | [1853-1871] | [1570-1588] |
| 406 | GUCACCGAUAUUCCAGUCU | AGACUGGAAUAUCGGUGAC | GP | [1837-1855] | [1554-1572] |
| 407 | CGCAAAUGCUGUCACCGAU | AUCGGUGACAGCAUUUGCG | GP | [1827-1845] | [1544-1562] |
| 408 | AGGGCUUUCGAACAACAAU | AUUGUUGUUCGAAAGCCCU | | [1305-1323] | [1169-1187] |
| 409 | CCAUUCAUAUCCGAGCAGC | GCUGCUCGGAUAUGAAUGG | | [1261-1279] | [1125-1143] |
| 410 | GAAGAUUUCUUCUCCAUUC | GAAUGGAGAAGAAAUCUUC | | [1248-1266] | [1112-1130] |
| 411 | CUCCAGAGGAAGAUUUCUU | AAGAAAUCUUCCUCUGGAG | | [1240-1258] | [1104-1122] |
| 412 | GGAAUGGCAUCCUUUUACU | AGUAAAAGGAUGCCAUUCC | | [1211-1229] | [1075-1093] |
| 413 | UGGAAUUGCAGAUGAACAA | UUGUUCAUCUGCAAUUCCA | | [1129-1147] | [993-1011] |
| 414 | CACAGUUCUGUCCAGAGAA | UUCUCUGGACAGAACUGUG | | [9-27] | — |
| 415 | GCACCCAUCCAAAGUUUUG | CAAAACUUUGGAUGGGUGC | | [1112-1130] | [976-994] |
| 416 | AUGCACCCAUCCAAAGUUU | AAACUUUGGAUGGGUGCAU | | [1110-1128] | [974-992] |
| 417 | GGUUGUGAUUACCAAGGUU | AACCUUGGUAAUCACAACC | | [1088-1106] | [952-970] |
| 418 | GUGAAAGGAUCCUCCGGUU | AACCGGAGGAUCCUUUCAC | | [1051-1069] | [915-933] |
| 419 | CUGGACAAAUGUUCCAUUC | GAAUGGAACAUUUGUCCAG | | [113-131] | — |
| 420 | UUCAUCCGGAGGAGUUAUU | AAUAACUCCUCCGGAUGAA | | [789-807] | [653-671] |
| 421 | GCCUUGAUUCUCAUGGUAA | UUACCAUGAGAAUCAAGGC | GP, Rb | [756-774] | [620-638] |
| 422 | UGGUCUCACUGGAGUGAUC | GAUCACUCCAGUGAGACCA | | [728-746] | [592-610] |
| 423 | GUGUGUUUUUCACAUCUUA | UAAGAUGUGAAAAACACAC | | [75-93] | — |
| 424 | CGCACACUGAGAAAGCAAU | AUUGCUUUCUCAGUGUGCG | | [468-486] | [332-350] |
| 425 | GGCGGAAUAAACUUAUUCA | UGAAUAAGUUUAUUCCGCC | | [32-50] | — |
| 426 | UACAUGUUUACUGUCACUU | AAGUGACAGUAAACAUGUA | | [2486-2504] | — |
| 427 | CCAUAUAACACCAACAGUG | CACUGUUGGUGUUAUAUGG | | [2467-2485] | — |
| 428 | CCCACAGAAGAUUUCCUAA | UUAGGAAAUCUUCUGUGGG | | [2352-2370] | — |
| 429 | ACUAAUCCCAGGUGAUUUU | AAAAUCACCUGGGAUUAGU | | [2274-2292] | — |
| 430 | GACUGUAGCAAGGCUUGAU | AUCAAGCCUUGCUACAGUC | | [2225-2243] | — |
| 431 | AUCCUAGAAAGGUUCAAUU | AAUUGAACCUUUCUAGGAU | | [1858-1876] | [1575-1593] |
| 432 | CAGUCUGGAUCCUAGAAAG | CUUUCUAGGAUCCAGACUG | | [1850-1868] | [1567-1585] |
| 433 | GAAUCUUCCCUGUUGCCUA | UAGGCAACAGGGAAGAUUC | | [162-180] | [26-44] |
| 434 | UCCCAGGAUUGAAGUGGAU | AUCCACUUCAAUCCUGGGA | | [1334-1352] | [1198-1216] |
| 435 | CCAAUUCCCAGGAUUGAAG | CUUCAAUCCUGGGAAUUGG | | [1329-1347] | [1193-1211] |
| 436 | GGACUGGACAGAAAAUCUC | GAGAUUUUCUGUCCAGUCC | Rat | [1283-1301] | [1147-1165] |
| 437 | GAGGAAGAUUUCUUCUCCA | UGGAGAAGAAAUCUUCCUC | | [1245-1263] | [1109-1127] |
| 438 | CCAGAGGAAGAUUUCUUCU | AGAAGAAAUCUUCCUCUGG | | [1242-1260] | [1106-1124] |
| 439 | CGCAAGUGUGCAGAGUCUU | AAGACUCUGCACACUUGCG | | [927-945] | [791-809] |
| 440 | GAGCAUGAAUGAGAGUCAU | AUGACUCUCAUUCAUGCUC | | [905-923] | [769-787] |
| 441 | GGUAUACUCACCACCUUUU | AAAAGGUGGUGAGUAUACC | | [820-838] | [684-702] |
| 442 | CAUGACAAUAGCCUUGAUU | AAUCAAGGCUAUUGUCAUG | | [746-764] | [610-628] |
| 443 | UCAUGACAAUAGCCUUGAU | AUCAAGGCUAUUGUCAUGA | | [745-763] | [609-627] |
| 444 | UGAUCAUGACAAUAGCCUU | AAGGCUAUUGUCAUGAUCA | | [742-760] | [606-624] |
| 445 | GUCUCACUGGAGUGAUCAU | AUGAUCACUCCAGUGAGAC | | [730-748] | [594-612] |
| 446 | GAGUAUGUGACAUUCACCA | UGGUGAAUGUCACAUACUC | | [702-720] | [566-584] |
| 447 | CAGCCUAUCUCAUGAUGAG | CUCAUCAUGAGAUAGGCUG | | [632-650] | [496-514] |
| 448 | AGCAAUUGGAUCACAACCU | AGGUUGUGAUCCAAUUGCU | | [481-499] | [345-363] |
| 449 | ACUUAUUCAUUCCCAGGAA | UUCCUGGGAAUGAAUAAGU | | [42-60] | — |
| 450 | UCAGUUUUGUUUCUGGUUG | CAACCAGAAACAAAACUGA | | [237-255] | [101-119] |
| 451 | UCCUGAGUUUUGGUUUGUA | UACAAACCAAAACUCAGGA | | [2314-2332] | — |
| 452 | GAACUUCUGAAGUUCUUAA | UUAAGAACUUCAGAAGUUC | | [2136-2154] | — |
| 453 | GAUCCUAGAAAGGUUCAAU | AUUGAACCUUUCUAGGAUC | | [1857-1875] | [1574-1592] |
| 454 | CCGAUAUUCCAGUCUGGAU | AUCCAGACUGGAAUAUCGG | | [1841-1859] | [1558-1576] |
| 455 | AGACCAAUGUGGGACAAUG | CAUUGUCCCACAUUGGUCU | Rat, Ms | [1725-1743] | [1442-1460] |
| 456 | GAAUCCGGAUUGCUGAAUC | GAUUCAGCAAUCCGGAUUC | | [148-166] | [12-30] |
| 457 | CCAGUAUGAAGUGGCUGUG | CACAGCCACUUCAUACUGG | | [1385-1403] | [1249-1267] |
| 458 | UUCACCAAUUCCCAGGAUU | AAUCCUGGGAAUUGGUGAA | | [1325-1343] | [1189-1207] |
| 459 | CAGAGGAAGAUUUCUUCUC | GAGAAGAAAUCUUCCUCUG | | [1243-1261] | [1107-1125] |
| 460 | CUGGAAUGGCAUCCUUUUA | UAAAAGGAUGCCAUUCCAG | | [1209-1227] | [1073-1091] |
| 461 | CCUGGAAUGGCAUCCUUUU | AAAAGGAUGCCAUUCCAGG | | [1208-1226] | [1072-1090] |
| 462 | UGCACCCAUCCAAAGUUUU | AAAACUUUGGAUGGGUGCA | | [1111-1129] | [975-993] |
| 463 | UGAGAGUCAUCCUCGCAAG | CUUGCGAGGAUGACUCUCA | | [914-932] | [778-796] |
| 464 | CUCAUGGUAACUUCAGCUA | UAGCUGAAGUUACCAUGAG | GP, Rat | [765-783] | [629-647] |
| 465 | AGGUGUGUGUUUUUCACAU | AUGUGAAAAACACACACCU | | [71-89] | — |
| 466 | GCCUACAUACAGCUAUUCA | UGAAUAGCUGUAUGUAGGC | | [532-550] | [396-414] |
| 467 | GGCCUAUAUGAUCUGCCUA | UAGGCAGAUCAUAUAGGCC | | [518-536] | [382-400] |
| 468 | UCACCUUCCACAAGCUGGU | ACCAGCUUGUGGAAGGUGA | | [499-517] | [363-381] |
| 469 | UGCUCAUUUUGCAGCCGCA | UGCGGCUGCAAAAUGAGCA | | [453-471] | [317-335] |
| 470 | GCACCUGCUCAUUUUGCAG | CUGCAAAAUGAGCAGGUGC | | [448-466] | [312-330] |
| 471 | UGGGUCAACAUUGGCCUGU | ACAGGCCAAUGUUGACCCA | | [344-362] | [208-226] |
| 472 | ACAGUGUACAUGUUUACUG | CAGUAAACAUGUACACUGU | | [2480-2498] | — |
| 473 | CAUCUCCUGAGUUUUGGUU | AACCAAAACUCAGGAGAUG | | [2310-2328] | — |
| 474 | CUAAUCCCAGGUGAUUUUA | UAAAAUCACCUGGGAUUAG | | [2275-2293] | — |
| 475 | CCAUGACUGUAGCAAGGCU | AGCCUUGCUACAGUCAUGG | | [2221-2239] | — |
| 476 | UGGACAGCCAUGACUGUAG | CUACAGUCAUGGCUGUCCA | | [2214-2232] | — |
| 477 | GAAUGUGCCUCUCAAGCCU | AGGCUUGAGAGGCACAUUC | | [2054-2072] | [1714-1732] |
| 478 | CCUCGGACUUUGGCAAAGA | UCUUUGCCAAAGUCCGAGG | | [1803-1821] | [1520-1538] |
| 479 | CAGGUCUGAAACAGAAAAC | GUUUUCUGUUUCAGACCUG | GP, Rat | [1696-1714] | [1413-1431] |
| 480 | GGUUCAACAACCUGUUGAC | GUCAACAGGUUGUUGAACC | | [1546-1564] | — |
| 481 | GCUGGUUGGAGCAGGAAUU | AAUUCCUGCUCCAACCAGC | | [1403-1421] | [1267-1285] |
| 482 | CUGAAGGACCUCUCCAGAA | UUCUGGAGAGGUCCUUCAG | | [132-150] | [1-14] |
| | | | | | (14/14) |
| 483 | AGAUGAACAAGCGUGGCUU | AAGCCACGCUUGUUCAUCU | | [1138-1156] | [1002-1020] |
| 484 | AGGUUGUGAUUACCAAGGU | ACCUUGGUAAUCACAACCU | | [1087-1105] | [951-969] |
| 485 | GCAGAAGGUUGUGAUUACC | GGUAAUCACAACCUUCUGC | | [1082-1100] | [946-964] |
| 486 | AAAGGAUCCUCCGGUUUUA | UAAAACCGGAGGAUCCUUU | | [1054-1072] | [918-936] |
| 487 | UCGCAAGUGUGCAGAGUCU | AGACUCUGCACACUUGCGA | | [926-944] | [790-808] |
| 488 | CCUGCGCUGGACAAAUGUU | AACAUUUGUCCAGCGCAGG | | [107-125] | — |
| 489 | CCUUGAUUCUCAUGGUAAC | GUUACCAUGAGAAUCAAGG | GP, Rb | [757-775] | [621-639] |
| 490 | UGGAGUAUGUGACAUUCAC | GUGAAUGUCACAUACUCCA | | [700-718] | [564-582] |
| 491 | CUAUAGCAGAAGCCGACAG | CUGUCGGCUUCUGCUAUAG | | [581-599] | [445-463] |
| 492 | CAAGAAAAAUCCUUGGGUC | GACCCAAGGAUUUUUCUUG | | [331-349] | [195-213] |
| 493 | ACAAGAAAAAUCCUUGGGU | ACCCAAGGAUUUUUCUUGU | | [330-348] | [194-212] |
| 494 | UCCUGUUUGUGGAUGCCUU | AAGGCAUCCACAAACAGGA | | [277-295] | [141-159] |
| 495 | CAGUGUACAUGUUUACUGU | ACAGUAAACAUGUACACUG | | [2481-2499] | — |
| 496 | CAACUAAUCCCAGGUGAUU | AAUCACCUGGGAUUAGUUG | | [2272-2290] | — |
| 497 | GGCUUGAUAGCAGAGGUUU | AAACCUCUGCUAUCAAGCC | | [2236-2254] | — |
| 498 | AGCAAGGCUUGAUAGCAGA | UCUGCUAUCAAGCCUUGCU | | [2231-2249] | — |
| 499 | AGCCUUGACUCCCUGGUAU | AUACCAGGGAGUCAAGGCU | | [2068-2086] | — |
| 500 | GACAGGUCUGAAACAGAAA | UUUCUGUUUCAGACCUGUC | GP, Rat | [1694-1712] | [1411-1429] |
| 501 | UGACAGGUCUGAAACAGAA | UUCUGUUUCAGACCUGUCA | GP, Rat | [1693-1711] | [1410-1428] |
| 502 | UCUUCCCUGUUGCCUAGAA | UUCUAGGCAACAGGGAAGA | | [165-183] | [29-47] |
| 503 | GUCAUGCAGCAUUAAACUU | AAGUUUAAUGCUGCAUGAC | GP, Rat, Ms | [1654-1672] | [1371-1389] |
| 504 | CAGGUGCCUUUUCCUGGUU | AACCAGGAAAAGGCACCUG | GP | [1531-1549] | — |
| 505 | CCGGAUUGCUGAAUCUUCC | GGAAGAUUCAGCAAUCCGG | | [152-170] | [16-34] |
| 506 | GUGCAGACCACAACCUCAA | UUGAGGUUGUGGUCUGCAC | GP | [1474-1492] | [1338-1356] |
| 507 | UUGCUUCUAUCUUGAAAUC | GAUUUCAAGAUAGAAGCAA | | [1435-1453] | [1299-1317] |
| 508 | CCAGAAUCCGGAUUGCUGA | UCAGCAAUCCGGAUUCUGG | | [145-163] | [9-27] |
| 509 | GAGGAUGUUUUCCAGUAUG | CAUACUGGAAAACAUCCUC | Ms | [1374-1392] | [1238-1256] |
| 510 | ACUGGACAGAAAAUCUCAU | AUGAGAUUUUCUGUCCAGU | Rat | [1285-1303] | [1149-1167] |
| 511 | AAGAUUUCUUCUCCAUUCA | UGAAUGGAGAAGAAAUCUU | | [1249-1267] | [1113-1131] |
| 512 | AGAGGAAGAUUUCUUCUCC | GGAGAAGAAAUCUUCCUCU | | [1244-1262] | [1108-1126] |
| 513 | UCCAGAGGAAGAUUUCUUC | GAAGAAAUCUUCCUCUGGA | | [1241-1259] | [1105-1123] |
| 514 | CUCUGCUCCAGAGGAAGAU | AUCUUCCUCUGGAGCAGAG | | [1235-1253] | [1099-1117] |
| 515 | UCCUGGAAUGGCAUCCUUU | AAAGGAUGCCAUUCCAGGA | | [1207-1225] | [1071-1089] |
| 516 | GGAAUUGCAGAUGAACAAG | CUUGUUCAUCUGCAAUUCC | | [1130-1148] | [994-1012] |
| 517 | AUCCAAAGUUUUGGAAUUG | CAAUUCCAAAACUUUGGAU | | [1118-1136] | [982-1000] |
| 518 | GAUCCUUGCACCGGUCAUU | AAUGACCGGUGCAAGGAUC | | [1022-1040] | [886-904] |
| 519 | GAGUCAUCCUCGCAAGUGU | ACACUUGCGAGGAUGACUC | | [917-935] | [781-799] |
| 520 | UCACUGGAGUGAUCAUGAC | GUCAUGAUCACUCCAGUGA | | [733-751] | [597-615] |
| 521 | GUAGGUGUGUGUUUUUCAC | GUGAAAAACACACACCUAC | | [69-87] | — |
| 522 | UGCCUACAUACAGCUAUUC | GAAUAGCUGUAUGUAGGCA | | [531-549] | [395-413] |
| 523 | CCGCACACUGAGAAAGCAA | UUGCUUUCUCAGUGUGCGG | | [467-485] | [331-349] |
| 524 | GUGGUUAACCACUGGUUUU | AAAACCAGUGGUUAACCAC | | [219-237] | [83-101] |
| 525 | GGUGGUUAACCACUGGUUU | AAACCAGUGGUUAACCACC | | [218-236] | [82-100] |
| 526 | GCAAGGCUUGAUAGCAGAG | CUCUGCUAUCAAGCCUUGC | | [2232-2250] | — |
| 527 | CUUAAGAACUUCUGAAGUU | AACUUCAGAAGUUCUUAAG | | [2131-2149] | — |
| 528 | CGUUACUUGAGCUUCAGCA | UGCUGAAGCUCAAGUAACG | | [2111-2129] | — |
| 529 | GGUUCAAUUCUACUUCAAC | GUUGAAGUAGAAUUGAACC | Rat | [1868-1886] | [1585-1603] |
| 530 | CCAAUGUGGGACAAUGAGU | ACUCAUUGUCCCACAUUGG | Rat, Ms | [1728-1746] | [1445-1463] |
| 531 | UCAACAACCUGUUGACUUC | GAAGUCAACAGGUUGUUGA | | [1549-1567] | — |
| 532 | UGGUUCAACAACCUGUUGA | UCAACAGGUUGUUGAACCA | | [1545-1563] | — |
| 533 | GCUGAUAGCACAGUUCUGU | ACAGAACUGUGCUAUCAGC | | [1-19] | — |
| 534 | CUGCUCCAGAGGAAGAUUU | AAAUCUUCCUCUGGAGCAG | | [1237-1255] | [1101-1119] |
| 535 | UCCAAAGUUUUGGAAUUGC | GCAAUUCCAAAACUUUGGA | | [1119-1137] | [983-1001] |
| 536 | CCAAGGUUGUUAUGCACCC | GGGUGCAUAACAACCUUGG | | [1099-1117] | [963-981] |
| 537 | GCAUGAAUGAGAGUCAUCC | GGAUGACUCUCAUUCAUGC | | [907-925] | [771-789] |
| 538 | UGGCCUAUAUGAUCUGCCU | AGGCAGAUCAUAUAGGCCA | | [517-535] | [381-399] |
| 539 | GCUUGAAUUUUAACAGCAC | GUGCUGUUAAAAUUCAAGC | | [382-400] | [246-264] |
| 540 | UGCUUGAAUUUUAACAGCA | UGCUGUUAAAAUUCAAGCA | Ms | [381-399] | [245-263] |
| 541 | CUCUGCUUGAAUUUUAACA | UGUUAAAAUUCAAGCAGAG | | [378-396] | [242-260] |
| 542 | AGGCGGAAUAAACUUAUUC | GAAUAAGUUUAUUCCGCCU | | [31-49] | — |
| 543 | CACUGGUUUUCAGUUUUGU | ACAAAACUGAAAACCAGUG | | [228-246] | [92-110] |
| 544 | AUGGAUCCUUUCUCAGAAA | UUUCUGAGAAAGGAUCCAU | | [2177-2195] | — |
| 545 | GAAACUGGGUGGUUAACCA | UGGUUAACCACCCAGUUUC | | [211-229] | [75-93] |
| 546 | UUGAGCUUCAGCAACUUAA | UUAAGUUGCUGAAGCUCAA | | [2117-2135] | — |
| 547 | CCACCUCUUGACAAUGGGA | UCCCAUUGUCAAGAGGUGG | | [194-212] | [58-76] |
| 548 | CUGGAUCCUAGAAAGGUUC | GAACCUUUCUAGGAUCCAG | | [1854-1872] | [1571-1589] |
| 549 | GGACUUUGGCAAAGAGCCU | AGGCUCUUUGCCAAAGUCC | | [1807-1825] | [1524-1542] |
| 550 | UGAAACAGAAAACCUCCUU | AAGGAGGUUUUCUGUUUCA | Rat | [1702-1720] | [1419-1437] |
| 551 | CUGAAACAGAAAACCUCCU | AGGAGGUUUUCUGUUUCAG | Rat | [1701-1719] | [1418-1436] |
| 552 | CUGUUGACUUCCCUGGAAC | GUUCCAGGGAAGUCAACAG | | [1557-1575] | — |
| 553 | CCAGUGAGGAUGUUUUCCA | UGGAAAACAUCCUCACUGG | | [1369-1387] | [1233-1251] |
| 554 | CAGCCAGUGAGGAUGUUUU | AAAACAUCCUCACUGGCUG | | [1366-1384] | [1230-1248] |
| 555 | CACAGCCAGUGAGGAUGUU | AACAUCCUCACUGGCUGUG | | [1364-1382] | [1228-1246] |
| 556 | ACCAAUUCCCAGGAUUGAA | UUCAAUCCUGGGAAUUGGU | | [1328-1346] | [1192-1210] |
| 557 | UGGAAUGGCAUCCUUUUAC | GUAAAAGGAUGCCAUUCCA | | [1210-1228] | [1074-1092] |
| 558 | ACCCAUCCAAAGUUUUGGA | UCCAAAACUUUGGAUGGGU | | [1114-1132] | [978-996] |
| 559 | CAAGGUUGUUAUGCACCCA | UGGGUGCAUAACAACCUUG | | [1100-1118] | [964-982] |
| 560 | UGGUAUACUCACCACCUUU | AAAGGUGGUGAGUAUACCA | | [819-837] | [683-701] |
| 561 | GUGACAUUCACCAGCGUUG | CAACGCUGGUGAAUGUCAC | | [708-726] | [572-586] |
| | | | | | (15/15 |
| 562 | UCUGCCUACAUACAGCUAU | AUAGCUGUAUGUAGGCAGA | | [529-547] | [393-411] |
| 563 | CUUUUGAUAUGGUCUUAUC | GAUAAGACCAUAUCAAAAG | | [2502-2520] | — |
| 564 | UGGUUAACCACUGGUUUUC | GAAAACCAGUGGUUAACCA | | [220-238] | [84-102] |
| 565 | CAUGGAUCCUUUCUCAGAA | UUCUGAGAAAGGAUCCAUG | | [2176-2194] | — |
| 566 | AGAACUUCUGAAGUUCUUA | UAAGAACUUCAGAAGUUCU | | [2135-2153] | — |
| 567 | AAGAACUUCUGAAGUUCUU | AAGAACUUCAGAAGUUCUU | | [2134-2152] | — |
| 568 | CUCCAAACCACCUCUUGAC | GUCAAGAGGUGGUUUGGAG | | [187-205] | [51-69] |
| 569 | UGCCUAGAAGGGCUCCAAA | UUUGGAGCCCUUCUAGGCA | | [175-193] | [39-57] |
| 570 | CGUGACAGGUCUGAAACAG | CUGUUUCAGACCUGUCACG | | [1691-1709] | [1408-1426] |
| 571 | CUGACAUCGUGACAGGUCU | AGACCUGUCACGAUGUCAG | | [1684-1702] | [1401-1419] |
| 572 | UGACUUCCCUGGAACAGGA | UCCUGUUCCAGGGAAGUCA | | [1561-1579] | — |
| 573 | UCCAGUAUGAAGUGGCUGU | ACAGCCACUUCAUACUGGA | Ms | [1384-1402] | [1248-1266] |
| 574 | CAGUGAGGAUGUUUUCCAG | CUGGAAAACAUCCUCACUG | Ms | [1370-1388] | [1234-1252] |
| 575 | AGGUUGUUAUGCACCCAUC | GAUGGGUGCAUAACAACCU | | [1102-1120] | [966-984] |
| 576 | AAGGUUGUUAUGCACCCAU | AUGGGUGCAUAACAACCUU | | [1101-1119] | [965-983] |
| 577 | CCCAGCAGAAGGUUGUGAU | AUCACAACCUUCUGCUGGG | | [1078-1096] | [942-960] |
| 578 | CCUUGCACCGGUCAUUCUU | AAGAAUGACCGGUGCAAGG | | [1025-1043] | [889-907] |
| 579 | GUGAUCAUGACAAUAGCCU | AGGCUAUUGUCAUGAUCAC | | [741-759] | [605-623] |
| 580 | CUGGUCUCACUGGAGUGAU | AUCACUCCAGUGAGACCAG | | [727-745] | [591-609] |
| 581 | UGCUUCCUGUGUGUCGCAA | UUGCGACACACAGGAAGCA | | [409-427] | [273-291] |
| 582 | CUGCUUGAAUUUUAACAGC | GCUGUUAAAAUUCAAGCAG | | [380-398] | [244-262] |
| 583 | CCAUCUCCUGAGUUUUGGU | ACCAAAACUCAGGAGAUGG | | [2309-2327] | — |
| 584 | CAUGACUGUAGCAAGGCUU | AAGCCUUGCUACAGUCAUG | | [2222-2240] | — |
| 585 | ACUUGAGCUUCAGCAACUU | AAGUUGCUGAAGCUCAAGU | | [2115-2133] | — |
| 586 | CCUAGAAAGGUUCAAUUCU | AGAAUUGAACCUUUCUAGG | | [1860-1878] | [1577-1595] |
| 587 | UCCAGUCUGGAUCCUAGAA | UUCUAGGAUCCAGACUGGA | | [1848-1866] | [1565-1583] |
| 588 | AGGUGCCUUUUCCUGGUUC | GAACCAGGAAAAGGCACCU | GP | [1532-1550] | — |
| 589 | ACAGGUGCCUUUUCCUGGU | ACCAGGAAAAGGCACCUGU | GP | [1530-1548] | — |
| 590 | GGAUUGAAGUGGAUGGUCC | GGACCAUCCACUUCAAUCC | | [1339-1357] | [1203-1221] |
| 591 | UGGAAGUGGAUCCUUGCAC | GUGCAAGGAUCCACUUCCA | Ms | [1014-1032] | [878-896] |
| 592 | UGACAAUAGCCUUGAUUCU | AGAAUCAAGGCUAUUGUCA | | [748-766] | [612-630] |
| 593 | GUUCUUGGCUAAAUCCCAU | AUGGGAUUUAGCCAAGAAC | | [661-679] | [525-543] |
| 594 | GUCUUAUCCAGUGUGAACA | UGUUCACACUGGAUAAGAC | | [2513-2531] | — |
| 595 | UGGUCUUAUCCAGUGUGAA | UUCACACUGGAUAAGACCA | | [2511-2529] | — |
| 596 | CCAGUGUUACCAUCUCCUG | CAGGAGAUGGUAACACUGG | | [2300-2318] | — |
| 597 | AGCUUCAGCAACUUAAGAA | UUCUUAAGUUGCUGAAGCU | | [2120-2138] | — |
| 598 | CCUCCUUUGGGAGACCAAU | AUUGGUCUCCCAAAGGAGG | Rat | [1714-1732] | [1431-1449] |
| 599 | ACAGCCAGUGAGGAUGUUU | AAACAUCCUCACUGGCUGU | | [1365-1383] | [1229-1247] |
| 600 | UCCUUGGCUUAGGGAUUCA | UGAAUCCCUAAGCCAAGGA | | [850-868] | [714-732] |
| 601 | CACAAGCUGGUGGCCUAUA | UAUAGGCCACCAGCUUGUG | | [507-525] | [371-389] |
| 602 | ACCAACAGUGUACAUGUUU | AAACAUGUACACUGUUGGU | | [2476-2494] | — |
| 603 | UGCCUCUCAAGCCUUGACU | AGUCAAGGCUUGAGAGGCA | | [2059-2077] | [1719-1734] |
| | | | | | (16/16) |
| 604 | ACCUCCUUUGGGAGACCAA | UUGGUCUCCCAAAGGAGGU | Rat | [1713-1731] | [1430-1448] |
| 605 | CAGAAAACCUCCUUUGGGA | UCCCAAAGGAGGUUUUCUG | Rat | [1707-1725] | [1424-1442] |
| 606 | ACCGGAUGGGACAGCAAUA | UAUUGCUGUCCCAUCCGGU | | [1629-1647] | — |
| 607 | CCUUGGCUUAGGGAUUCAC | GUGAAUCCCUAAGCCAAGG | | [851-869] | [715-733] |
| 608 | CUGGUUUUCAGUUUUGUUU | AAACAAAACUGAAAACCAG | | [230-248] | [94-112] |
| 609 | CAACAGUGUACAUGUUUAC | GUAAACAUGUACACUGUUG | | [2478-2496] | — |
| 610 | AGGUGAUUUUAUCAAUUCC | GGAAUUGAUAAAAUCACCU | | [2283-2301] | — |
| 611 | UAAAGCCCAUGGAUCCUUU | AAAGGAUCCAUGGGCUUUA | | [2169-2187] | — |
| 612 | GUCUGAAACAGAAAACCUC | GAGGUUUUCUGUUUCAGAC | Rat | [1699-1717] | [1416-1434] |
| 613 | GUCUUGGAAGUGGAUCCUU | AAGGAUCCACUUCCAAGAC | Ms | [1010-1028] | [874-892] |
| 614 | GGAUGUUUUCCAGUAUGAA | UUCAUACUGGAAAACAUCC | Ms | [1376-1394] | [1240-1258] |
| 615 | UCCUUUGGGAGACCAAUGU | ACAUUGGUCUCCCAAAGGA | Rat, Ms | [1716-1734] | [1433-1451] |
| 616 | ACAGCCAUGACUGUAGCAA | UUGCUACAGUCAUGGCUGU | | [2217-2235] | — |
| 617 | AGGGAGACAGGUGCCUUUU | AAAAGGCACCUGUCUCCCU | GP | [1524-1542] | — |
| 618 | AGGAUGUUUUCCAGUAUGA | UCAUACUGGAAAACAUCCU | Ms | [1375-1393] | [1239-1257] |
| 619 | GGUAACUUCAGCUACUGAG | CUCAGUAGCUGAAGUUACC | | [770-788] | [634-652] |

TABLE A4
NOX1 19-mer oligomer pairs - NADPH oxidase 1

| | | | | | |
|---|---|---|---|---|---|
| | | | | Human- | Human- |
| | | | | 148536872 | 148536874 |
| No. | Sense siRNA | AntiSense siRNA | Other Sp | ORF: 207-1901 | ORF: 207-1754 |

| | | | | | |
|---|---|---|---|---|---|
| 1 | GGGUGACUUUUUAAAUAAA | UUUAUUUAAAAAGUCACCC | | [2316-2334] | [2169-2187] |
| 2 | GGCAGUAUAUCUUUGUUAA | UUAACAAAGAUAUACUGCC | | [1171-1189] | [1171-1189] |
| 3 | GGUGACUUUUUAAAUAAAA | UUUUAUUUAAAAAGUCACC | | [2317-2335] | [2170-2188] |
| 4 | CAAUGAGUUUUCUACAAUA | UAUUGUAGAAAACUCAUUG | | [1739-1757] | [1592-1610] |
| 5 | CAGUGUGAACAGCAAUUUA | UAAAUUGCUGUUCACACUG | | [2463-2481] | [2316-2334] |
| 6 | AGAAGGUUGUGAUUACCAA | UUGGUAAUCACAACCUUCU | | [1084-1102] | [1084-1102] |
| 7 | GGUGGUUCAGAGUUAUACA | UGUAUAACUCUGAACCACC | | [2198-2216] | [2051-2069] |
| 8 | CAUCGUGACAGGUCUGAAA | UUUCAGACCUGUCACGAUG | | [1688-1706] | [1541-1559] |
| 9 | AGGGUGACUUUUUAAAUAA | UUAUUUAAAAAGUCACCCU | | [2315-2333] | [2168-2186] |
| 10 | GAGAAGGAAGGCAGAAUAA | UUAUUCUGCCUUCCUUCUC | | [23-41] | [23-41] |
| 11 | GACCAAUGUGGGACAAUGA | UCAUUGUCCCACAUUGGUC | Rat, Ms | [1726-1744] | [1579-1597] |
| 12 | CACCGGUCAUUCUUUAUAU | AUAUAAAGAAUGACCGGUG | | [1030-1048] | [1030-1048] |
| 13 | GAAAGCAAUUGGAUCACAA | UUGUGAUCCAAUUGCUUUC | | [478-496] | [478-496] |
| 14 | CAUUCUUUAUAUCUGUGAA | UUCACAGAUAUAAAGAAUG | | [1037-1055] | [1037-1055] |
| 15 | GUGUUUUUCACAUCUUAAA | UUUAAGAUGUGAAAAACAC | | [77-95] | [77-95] |
| 16 | GAACAACAAUAUUCACCAA | UUGGUGAAUAUUGUUGUUC | | [1314-1332] | [1314-1332] |
| 17 | AAGUAGGGUGACUUUUUAA | UUAAAAAGUCACCCUACUU | | [2311-2329] | [2164-2182] |
| 18 | UAUUUUUGCUCAUCAAAAA | UUUUUGAUGAGCAAAAAUA | | [2486-2504] | [2339-2357] |
| 19 | GCUCUCUGCUUGAAUUUUA | UAAAAUUCAAGCAGAGAGC | | [375-393] | [375-393] |
| 20 | CAGAGGAGAGCAUGAAUGA | UCAUUCAUGCUCUCCUCUG | | [898-916] | [898-916] |
| 21 | AAGGGCUUUCGAACAACAA | UUGUUGUUCGAAAGCCCUU | | [1304-1322] | [1304-1322] |
| 22 | CAACAAAGAAAAUUUUUGA | UCAAAAAUUUUCUUUGUUG | | [1883-1901] | [1736-1754] |
| 23 | UAGGGUGACUUUUUAAAUA | UAUUUAAAAAGUCACCCUA | | [2314-2332] | [2167-2185] |
| 24 | UCAGAAAAAUAACUGUAAA | UUUACAGUUAUUUUUCUGA | | [2132-2150] | [1985-2003] |
| 25 | GUGUGAACAGCAAUUUAUU | AAUAAAUUGCUGUUCACAC | | [2465-2483] | [2318-2336] |
| 26 | GAUCGUGACUCCCACUGUA | UACAGUGGGAGUCACGAUC | | [960-978] | [960-978] |
| 27 | UCUUAUUUUUGCUCAUCAA | UUGAUGAGCAAAAAUAAGA | | [2483-2501] | [2336-2354] |
| 28 | GACAGUGGAGUAUGUGACA | UGUCACAUACUCCACUGUC | | [695-713] | [695-713] |
| 29 | GACAAUGAGUUUUCUACAA | UUGUAGAAAACUCAUUGUC | | [1737-1755] | [1590-1608] |
| 30 | GCAGUAUAUCUUUGUUAAU | AUUAACAAAGAUAUACUGC | | [1172-1190] | [1172-1190] |
| 31 | AGAAGGAAGGCAGAAUAAA | UUUAUUCUGCCUUCCUUCU | | [24-42] | [24-42] |
| 32 | CAGAAUCCGGAUUGCUGAA | UUCAGCAAUCCGGAUUCUG | | [146-164] | [146-164] |
| 33 | GCUUCUAUCUUGAAAUCCA | UGGAUUUCAAGAUAGAAGC | | [1437-1455] | [1437-1455] |
| 34 | CGUCUGCUCUCUGCUUGAA | UUCAAGCAGAGAGCAGACG | | [370-388] | [370-388] |
| 35 | UAUCCAGUGUGAACAGCAA | UUGCUGUUCACACUGGAUA | | [2459-2477] | [2312-2330] |
| 36 | GCAGAAUAAACUUAUUCAU | AUGAAUAAGUUUAUUCUGC | | [33-51] | [33-51] |
| 37 | AAUUCUACUUCAACAAAGA | UCUUUGUUGAAGUAGAAUU | Rat | [1873-1891] | [1726-1744] |
| 38 | UGUUAUGCACCCAUCCAAA | UUUGGAUGGGUGCAUAACA | | [1106-1124] | [1106-1124] |
| 39 | AGCAGUCACUUUAGGAUAA | UUAUCCUAAAGUGACUGCU | | [1978-1996] | [1831-1849] |
| 40 | CAUAAGGGCUUUCGAACAA | UUGUUCGAAAGCCCUUAUG | | [1301-1319] | [1301-1319] |
| 41 | AUAUGAGAAGGCCGACAAA | UUUGUCGGCCUUCUCAUAU | | [302-320] | [302-320] |
| 42 | UGUGGAUGCCUUCCUGAAA | UUUCAGGAAGGCAUCCACA | | [284-302] | [284-302] |
| 43 | AUGCCUUCCUGAAAUAUGA | UCAUAUUUCAGGAAGGCAU | | [289-307] | [289-307] |
| 44 | CAUUCAUAUCCGAGCAGCA | UGCUGCUCGGAUAUGAAUG | | [1262-1280] | [1262-1280] |
| 45 | GCAGAGGUUUGGUGGUUCA | UGAACCACCAAACCUCUGC | | [2188-2206] | [2041-2059] |
| 46 | AAGGCAGAAUAAACUUAUU | AAUAAGUUUAUUCUGCCUU | | [30-48] | [30-48] |
| 47 | CACAGAAGAUUUCUAAGUA | UACUUAGAAAUCUUCUGUG | Rat | [2297-2315] | [2150-2168] |
| 48 | UCCAGUGUGCAGACCACAA | UUGUGGUCUGCACACUGGA | | [1468-1486] | [1468-1486] |
| 49 | GUUCAAUUCUACUUCAACA | UGUUGAAGUAGAAUUGAAC | Rat | [1869-1887] | [1722-1740] |
| 50 | CAAACAGAGGAGAGCAUGA | UCAUGCUCUCCUCUGUUUG | | [894-912] | [894-912] |
| 51 | GGCAGAAUAAACUUAUUCA | UGAAUAAGUUUAUUCUGCC | | [32-50] | [32-50] |
| 52 | AUAAGAAUGUGCCUCUCAA | UUGAGAGGCACAUUCUUAU | | [1993-2011] | [1846-1864] |
| 53 | CAAUAUUCACCAAUUCCCA | UGGGAAUUGGUGAAUAUUG | | [1320-1338] | [1320-1338] |
| 54 | GAGGAGAGCAUGAAUGAGA | UCUCAUUCAUGCUCUCCUC | | [900-918] | [900-918] |
| 55 | GUUGGUCAUGCAGCAUUAA | UUAAUGCUGCAUGACCAAC | | [1650-1668] | [1503-1521] |
| 56 | CUGGUAUUCUUUUUUUGAU | AUCAAAAAAAGAAUACCAG | | [2023-2041] | [1876-1894] |
| 57 | GAAACACGACAGUGGAGUA | UACUCCACUGUCGUGUUUC | | [688-706] | [688-706] |
| 58 | CAUCUUAAAGGCUCACAGA | UCUGUGAGCCUUUAAGAUG | | [87-105] | [87-105] |
| 59 | UCUCAUAAGGGCUUUCGAA | UUCGAAAGCCCUUAUGAGA | | [1298-1316] | [1298-1316] |
| 60 | GCCGACAAAUACUACUACA | UGUAGUAGUAUUUGUCGGC | | [312-330] | [312-330] |
| 61 | UUUUGGAAUUGCAGAUGAA | UUCAUCUGCAAUUCCAAAA | | [1126-1144] | [1126-1144] |
| 62 | GGUCAUUCUUUAUAUCUGU | ACAGAUAUAAAGAAUGACC | | [1034-1052] | [1034-1052] |
| 63 | GUGACAGGUCUGAAACAGA | UCUGUUUCAGACCUGUCAC | | [1692-1710] | [1545-1563] |
| 64 | GGAGACCAAUGUGGGACAA | UUGUCCCACAUUGGUCUCC | Rat, Ms | [1723-1741] | [1576-1594] |
| 65 | GGACGGUAAUCUGCAUUUU | AAAAUGCAGAUUACCGUCC | | [1915-1933] | [1768-1786] |
| 66 | GCACCGGUCAUUCUUUAUA | UAUAAAGAAUGACCGGUGC | | [1029-1047] | [1029-1047] |
| 67 | GAAGGAAGGCAGAAUAAAC | GUUUAUUCUGCCUUCCUUC | | [25-43] | [25-43] |
| 68 | GAGUGAUCAUGACAAUAGC | GCUAUUGUCAUGAUCACUC | | [739-757] | [739-757] |
| 69 | UCUACUUCAACAAAGAAAA | UUUUCUUUGUUGAAGUAGA | | [1876-1894] | [1729-1747] |
| 70 | GAGCAGUCACUUUAGGAUA | UAUCCUAAAGUGACUGCUC | | [1977-1995] | [1830-1848] |
| 71 | AGAGAAGGAAGGCAGAAUA | UAUUCUGCCUUCCUUCUCU | | [22-40] | [22-40] |
| 72 | GACUGGACAGAAAAUCUCA | UGAGAUUUUCUGUCCAGUC | Rat | [1284-1302] | [1284-1302] |
| 73 | CCAGAGAAGGAAGGCAGAA | UUCUGCCUUCCUUCUCUGG | | [20-38] | [20-38] |
| 74 | GGUCAUGCAGCAUUAAACU | AGUUUAAUGCUGCAUGACC | Rat, Ms, GP | [1653-1671] | [1506-1524] |
| 75 | UGGAUGCCUUCCUGAAAUA | UAUUUCAGGAAGGCAUCCA | | [286-304] | [286-304] |
| 76 | CUUAUUUUUGCUCAUCAAA | UUUGAUGAGCAAAAAUAAG | | [2484-2502] | [2337-2355] |
| 77 | UUUGCUCAUCAAAAAAUAA | UUAUUUUUUGAUGAGCAAA | | [2490-2508] | [2343-2361] |
| 78 | UCAGAGUUAUACAACUAAU | AUUAGUUGUAUAACUCUGA | | [2204-2222] | [2057-2075] |
| 79 | CAGAAAAAUAACUGUAAAU | AUUUACAGUUAUUUUUCUG | | [2133-2151] | [1986-2004] |
| 80 | GGUUAGGGCUGAAUGUUUU | AAAACAUUCAGCCCUAACC | | [259-277] | [259-277] |
| 81 | CUCAGAAAAAUAACUGUAA | UUACAGUUAUUUUUCUGAG | | [2131-2149] | [1984-2002] |
| 82 | CUACUUCAACAAAGAAAAU | AUUUUCUUUGUUGAAGUAG | | [1877-1895] | [1730-1748] |
| 83 | GAUUUCUUCUCCAUUCAUA | UAUGAAUGGAGAAGAAAUC | | [1251-1269] | [1251-1269] |
| 84 | UGAAAUCCAUCUGGUACAA | UUGUACCAGAUGGAUUUCA | | [1447-1465] | [1447-1465] |
| 85 | AACUGUAAAUCUUUCUGGA | UCCAGAAAGAUUUACAGUU | | [2142-2160] | [1995-2013] |
| 86 | AAAUCCAUCUGGUACAAAU | AUUUGUACCAGAUGGAUUU | | [1449-1467] | [1449-1467] |
| 87 | UUUUUUUGAUUGCAUUCAA | UUGAAUGCAAUCAAAAAAA | | [2032-2050] | [1885-1903] |
| 88 | CACCAAUUCCCAGGAUUGA | UCAAUCCUGGGAAUUGGUG | | [1327-1345] | [1327-1345] |
| 89 | GUGGAUGCCUUCCUGAAAU | AUUUCAGGAAGGCAUCCAC | | [285-303] | [285-303] |
| 90 | GAUUCUCAUGGUAACUUCA | UGAAGUUACCAUGAGAAUC | Rb, GP | [761-779] | [761-779] |
| 91 | AUGAGAAGGCCGACAAAUA | UAUUUGUCGGCCUUCUCAU | | [304-322] | [304-322] |
| 92 | GAAGGCAGAAUAAACUUAU | AUAAGUUUAUUCUGCCUUC | | [29-47] | [29-47] |
| 93 | GCAAAGAGCCUGCGCAAAU | AUUUGCGCAGGCUCUUUGC | | [1815-1833] | [1668-1686] |
| 94 | CAGUGUUACCAUCUCCUGA | UCAGGAGAUGGUAACACUG | | [2244-2262] | [2097-2115] |
| 95 | UAGGAAUAAGGACGGUAAU | AUUACCGUCCUUAUUCCUA | | [1906-1924] | [1759-1777] |
| 96 | UGCUGUCACCGAUAUUCCA | UGGAAUAUCGGUGACAGCA | GP | [1833-1851] | [1686-1704] |
| 97 | UCUUGAAAUCCAUCUGGUA | UACCAGAUGGAUUUCAAGA | GP | [1444-1462] | [1444-1462] |
| 98 | ACACGACAGUGGAGUAUGU | ACAUACUCCACUGUCGUGU | | [691-709] | [691-709] |
| 99 | UCGUGACAGGUCUGAAACA | UGUUUCAGACCUGUCACGA | | [1690-1708] | [1543-1561] |
| 100 | GUAGGCGCCCUAAGUUUGA | UCAAACUUAGGGCGCCUAC | | [976-994] | [976-994] |
| 101 | GGAAGGCAGAAUAAACUUA | UAAGUUUAUUCUGCCUUCC | | [28-46] | [28-46] |
| 102 | AGACCACAACCUCAAAACA | UGUUUUGAGGUUGUGGUCU | | [1478-1496] | [1478-1496] |
| 103 | UCAUUGCACACCUGUUUAA | UUAAACAGGUGUGCAAUGA | | [553-571] | [553-571] |
| 104 | UUUUGCUCAUCAAAAAAUA | UAUUUUUUGAUGAGCAAAA | | [2489-2507] | [2342-2360] |
| 105 | CAUGGUAACUUCAGCUACU | AGUAGCUGAAGUUACCAUG | | [767-785] | [767-785] |
| 106 | CCUCAAUCUCUCUCCUGGA | UCCAGGAGAGAGAUUGAGG | GP | [1195-1213] | [1195-1213] |
| 107 | GGGCAGUAUAUCUUUGUUA | UAACAAAGAUAUACUGCCC | | [1170-1188] | [1170-1188] |
| 108 | GCCCAUGGAUCCUUUCUCA | UGAGAAAGGAUCCAUGGGC | | [2116-2134] | [1969-1987] |
| 109 | AGUGUGAACAGCAAUUUAU | AUAAAUUGCUGUUCACACU | | [2464-2482] | [2317-2335] |
| 110 | CUACAUACAGCUAUUCACA | UGUGAAUAGCUGUAUGUAG | | [534-552] | [534-552] |
| 111 | ACAUCGUGACAGGUCUGAA | UUCAGACCUGUCACGAUGU | | [1687-1705] | [1540-1558] |
| 112 | CAGAAGGUUGUGAUUACCA | UGGUAAUCACAACCUUCUG | | [1083-1101] | [1083-1101] |
| 113 | UGUGCAGAGUCUUUUGAGA | UCUCAAAAGACUCUGCACA | | [933-951] | [933-951] |
| 114 | UAAGGACGGUAAUCUGCAU | AUGCAGAUUACCGUCCUUA | | [1912-1930] | [1765-1783] |
| 115 | GUGAGGAUGUUUUCCAGUA | UACUGGAAAACAUCCUCAC | Ms | [1372-1390] | [1372-1390] |
| 116 | UAGGCGCCCUAAGUUUGAA | UUCAAACUUAGGGCGCCUA | | [977-995] | [977-995] |
| 117 | GUUUGGUGGUUCAGAGUUA | UAACUCUGAACCACCAAAC | | [2194-2212] | [2047-2065] |
| 118 | UAAGGGCUUUCGAACAACA | UGUUGUUCGAAAGCCCUUA | | [1303-1321] | [1303-1321] |
| 119 | AUCACAACCUCACCUUCCA | UGGAAGGUGAGGUUGUGAU | Rat, Ms | [490-508] | [490-508] |
| 120 | GAACAGCAAUUUAUUCUUA | UAAGAAUAAAUUGCUGUUC | | [2469-2487] | [2322-2340] |
| 121 | UGUUUAACUUUGACUGCUA | UAGCAGUCAAAGUUAAACA | | [565-583] | [565-583] |
| 122 | GAAGGUUGUGAUUACCAAG | CUUGGUAAUCACAACCUUC | | [1085-1103] | [1085-1103] |
| 123 | AGGGAUUCACGGCAUUGGU | ACCAAUGCCGUGAAUCCCU | | [860-878] | [860-878] |
| 124 | UAAACAAAAUUACCGAGAA | UUCUCGGUAAUUUUGUUUA | | [2382-2400] | [2235-2253] |
| 125 | GUUUAACUUUGACUGCUAU | AUAGCAGUCAAAGUUAAAC | | [566-584] | [566-584] |
| 126 | GUUACUUGAGCUUCAGCAA | UUGCUGAAGCUCAAGUAAC | | [2055-2073] | [1908-1926] |
| 127 | CACUGGAGUGAUCAUGACA | UGUCAUGAUCACUCCAGUG | | [734-752] | [734-752] |
| 128 | UUAUUUUUGCUCAUCAAAA | UUUUGAUGAGCAAAAAUAA | | [2485-2503] | [2338-2356] |
| 129 | GAGCUUCAGCAACUUAAGA | UCUUAAGUUGCUGAAGCUC | | [2062-2080] | [1915-1933] |
| 130 | CACCUUUUUAUCUUCUAUA | UAUAGAAGAUAAAAAGGUG | | [831-849] | [831-849] |
| 131 | CUCAAUCUCUCUCCUGGAA | UUCCAGGAGAGAGAUUGAG | | [1196-1214] | [1196-1214] |
| 132 | AGCUAUUCACAUCAUUGCA | UGCAAUGAUGUGAAUAGCU | | [542-560] | [542-560] |
| 133 | CAUGUUUACUGUCACUUUU | AAAAGUGACAGUAAACAUG | | [2430-2448] | [2283-2301] |
| 134 | GAUUCACGGCAUUGGUGGA | UCCACCAAUGCCGUGAAUC | | [863-881] | [863-881] |
| 135 | AGUUCUGUCCAGAGAAGGA | UCCUUCUCUGGACAGAACU | | [12-30] | [12-30] |
| 136 | CAAUGUGGGACAAUGAGUU | AACUCAUUGUCCCACAUUG | Rat, Ms | [1729-1747] | [1582-1600] |
| 137 | ACGACAGUGGAGUAUGUGA | UCACAUACUCCACUGUCGU | | [693-711] | [693-711] |
| 138 | GCAAAUGCUGUCACCGAUA | UAUCGGUGACAGCAUUUGC | GP | [1828-1846] | [1681-1699] |
| 139 | CUUCAACAAAGAAAAUUUU | AAAAUUUUCUUUGUUGAAG | | [1880-1898] | [1733-1751] |
| 140 | AUUCUACUUCAACAAAGAA | UUCUUUGUUGAAGUAGAAU | Rat | [1874-1892] | [1727-1745] |
| 141 | AGAGGUUUGGUGGUUCAGA | UCUGAACCACCAAACCUCU | | [2190-2208] | [2043-2061] |
| 142 | CAAAGAAAAUUUUUGAGUU | AACUCAAAAAUUUUCUUUG | | [1886-1904] | [1739-1757] |
| 143 | GCUCCAAACCACCUCUUGA | UCAAGAGGUGGUUUGGAGC | | [186-204] | [186-204] |
| 144 | AAGUUCUGAAGUUCUUAAA | UUUAAGAACUUCAGAACUU | | [2097-2115] | [1950-1968] |
| 145 | UUGCUCAUCAAAAAAUAAA | UUUAUUUUUUGAUGAGCAA | | [2491-2509] | [2344-2362] |
| 146 | AAUCCAUCUGGUACAAAUU | AAUUUGUACCAGAUGGAUU | | [1450-1468] | [1450-1468] |
| 147 | UUGGUCAUGCAGCAUUAAA | UUUAAUGCUGCAUGACCAA | | [1651-1669] | [1504-1522] |
| 148 | GGUUUGAGCAGUCACUUUA | UAAAGUGACUGCUCAAACC | | [1972-1990] | [1825-1843] |
| 149 | UCCAGAGAAGGAAGGCAGA | UCUGCCUUCCUUCUCUGGA | | [19-37] | [19-37] |
| 150 | CAAACCACCUCUUGACAAU | AUUGUCAAGAGGUGGUUUG | | [190-208] ORF | [190-208] ORF |
| 151 | ACAAUAGCCUUGAUUCUCA | UGAGAAUCAAGGCUAUUGU | | [750-768] | [750-768] |
| 152 | AACUUUGACUGCUAUAGCA | UGCUAUAGCAGUCAAAGUU | | [570-588] | [570-588] |
| 153 | GUCGCAAUCUGCUGUCCUU | AAGGACAGCAGAUUGCGAC | | [421-439] | [421-439] |
| 154 | CAGAAUAAACUUAUUCAUU | AAUGAAUAAGUUUAUUCUG | | [34-52] | [34-52] |
| 155 | AGAAGGCCGACAAAUACUA | UAGUAUUUGUCGGCCUUCU | | [307-325] | [307-325] |
| 156 | CUCACUGGAGUGAUCAUGA | UCAUGAUCACUCCAGUGAG | | [732-750] | [732-750] |
| 157 | UUGUGGAUGCCUUCCUGAA | UUCAGGAAGGCAUCCACAA | | [283-301] | [283-301] |
| 158 | AAUUUUUGAGUUAUAGGAA | UUCCUAUAACUCAAAAAUU | | [1893-1911] | [1746-1764] |
| 159 | UGGUAUUCUUUUUUUGAUU | AAUCAAAAAAAGAAUACCA | | [2024-2042] | [1877-1895] |
| 160 | ACAAAUGUUCCAUUCCUGA | UCAGGAAUGGAACAUUUGU | | [117-135] | [117-135] |
| 161 | AACAGAGGAGAGCAUGAAU | AUUCAUGCUCUCCUCUGUU | | [896-914] | [896-914] |
| 162 | UCCGGAGGAGUUAUUUUGA | UCAAAAUAACUCCUCCGGA | | [793-811] | [793-811] |
| 163 | AAACAGAGGAGAGCAUGAA | UUCAUGCUCUCCUCUGUUU | | [895-913] | [895-913] |
| 164 | GGUUAACCACUGGUUUUCA | UGAAAACCAGUGGUUAACC | | [221-239] | [221-239] |
| 165 | CAGCAUUGCUGGUCUCACU | AGUGAGACCAGCAAUGCUG | GP | [719-737] | [719-737] |
| 166 | CAUGAAUGAGAGUCAUCCU | AGGAUGACUCUCAUUCAUG | | [908-926] | [908-926] |
| 167 | GGUCAAACAGAGGAGAGCA | UGCUCUCCUCUGUUUGACC | | [891-909] | [891-909] |
| 168 | AUGUUUUCCUGUUUGUGGA | UCCACAAACAGGAAAACAU | | [271-289] | [271-289] |
| 169 | CAUGCAGCAUUAAACUUUG | CAAAGUUUAAUGCUGCAUG | Rat, Ms, GP | [1656-1674] | [1509-1527] |
| 170 | CAGUAUAUCUUUGUUAAUU | AAUUAACAAAGAUAUACUG | | [1173-1191] | [1173-1191] |
| 171 | GUACAAAUUCCAGUGUGCA | UGCACACUGGAAUUUGUAC | | [1460-1478] | [1460-1478] |
| 172 | GUUUCUGGUUGUUUGGUUA | UAACCAAACAACCAGAAAC | | [245-263] | [245-263] |
| 173 | ACAACCUCAAAACAAAAAA | UUUUUUGUUUUGAGGUUGU | | [1483-1501] | [1483-1501] |
| 174 | CUUUGACUGCUAUAGCAGA | UCUGCUAUAGCAGUCAAAG | | [572-590] | [572-590] |
| 175 | AUUUUUGCUCAUCAAAAAA | UUUUUUGAUGAGCAAAAAU | | [2487-2505] | [2340-2358] |
| 176 | CAAUUGGAUCACAACCUCA | UGAGGUUGUGAUCCAAUUG | | [483-501] | [483-501] |
| 177 | CAAAGAGCCUGCGCAAAUG | CAUUUGCGCAGGCUCUUUG | | [1816-1834] | [1669-1687] |
| 178 | UGAAGGACCUCUCCAGAAU | AUUCUGGAGAGGUCCUUCA | | [133-151] | [133-151] |
| 179 | CUUUGACCUCUGCUCCAGA | UCUGGAGCAGAGGUCAAAG | GP | [1228-1246] | [1228-1246] |
| 180 | AAGGAAGGCAGAAUAAACU | AGUUUAUUCUGCCUUCCUU | | [26-44] | [26-44] |
| 181 | AUGCAGCAUUAAACUUUGA | UCAAAGUUUAAUGCUGCAU | Rat, Ms, GP | [1657-1675] | [1510-1528] |
| 182 | AUGGGAAACUGGGUGGUUA | UAACCACCCAGUUUCCCAU | | [207-225] | [207-225] |
| 183 | GAAAUCCAUCUGGUACAAA | UUUGUACCAGAUGGAUUUC | | [1448-1466] | [1448-1466] |
| 184 | CAUCAUUGCACACCUGUUU | AAACAGGUGUGCAAUGAUG | | [551-569] | [551-569] |
| 185 | GCACACUGAGAAAGCAAUU | AAUUGCUUUCUCAGUGUGC | | [469-487] | [469-487] |
| 186 | UCCUUUCUCAGAAAAAUAA | UUAUUUUUCUGAGAAAGGA | | [2125-2143] | [1978-1996] |
| 187 | AACUUCUGAAGUUCUUAAA | UUUAAGAACUUCAGAAGUU | | [2080-2098] | [1933-1951] |
| 188 | AUCCAGUCCCGAAACACGA | UCGUGUUUCGGGACUGGAU | | [678-696] | [678-696] |
| 189 | AAAGUUCUGAAGUUCUUAA | UUAAGAACUUCAGAACUUU | | [2096-2114] | [1949-1967] |
| 190 | CAGCAGAAGGUUGUGAUUA | UAAUCACAACCUUCUGCUG | | [1080-1098] | [1080-1098] |
| 191 | AGGAAGGCAGAAUAAACUU | AAGUUUAUUCUGCCUUCCU | | [27-45] | [27-45] |
| 192 | UGAAGUGGAUGGUCCCUUU | AAAGGGACCAUCCACUUCA | | [1343-1361] | [1343-1361] |
| 193 | AUCUCAUAAGGGCUUUCGA | UCGAAAGCCCUUAUGAGAU | | [1297-1315] | [1297-1315] |
| 194 | CAACCUCACCUUCCACAAG | CUUGUGGAAGGUGAGGUUG | | [494-512] | [494-512] |
| 195 | AUUCUUUAUAUCUGUGAAA | UUUCACAGAUAUAAAGAAU | | [1038-1056] | [1038-1056] |
| 196 | AGUUCAUCCGGAGGAGUUA | UAACUCCUCCGGAUGAACU | | [787-805] | [787-805] |
| 197 | UGCUCAUCAAAAAAUAAAG | CUUUAUUUUUUGAUGAGCA | | [2492-2510] | [2345-2363] |
| 198 | UCAAUUCUACUUCAACAAA | UUUGUUGAAGUAGAAUUGA | Rat | [1871-1889] | [1724-1742] |
| 199 | CAAUUCUACUUCAACAAAG | CUUUGUUGAAGUAGAAUUG | Rat | [1872-1890] | [1725-1743] |
| 200 | UUCAGAGUUAUACAACUAA | UUAGUUGUAUAACUCUGAA | | [2203-2221] | [2056-2074] |
| 201 | CCAAACCACCUCUUGACAA | UUGUCAAGAGGUGGUUUGG | | [189-207] | [189-207] |
| 202 | GAAAAAUAACUGUAAAUCU | AGAUUUACAGUUAUUUUUC | | [2135-2153] | [1988-2006] |
| 203 | UUGCACCGGUCAUUCUUUA | UAAAGAAUGACCGGUGCAA | | [1027-1045] | [1027-1045] |
| 204 | GUUCUGUCCAGAGAAGGAA | UUCCUUCUCUGGACAGAAC | | [13-31] | [13-31] |
| 205 | GAAUAAGGACGGUAAUCUG | CAGAUUACCGUCCUUAUUC | | [1909-1927] | [1762-1780] |
| 206 | AGUUCUUAAAGCCCAUGGA | UCCAUGGGCUUUAAGAACU | | [2106-2124] | [1959-1977] |
| 207 | UUCCCAGGAUUGAAGUGGA | UCCACUUCAAUCCUGGGAA | | [1333-1351] | [1333-1351] |
| 208 | GGUCUUAUCCAGUGUGAAC | GUUCACACUGGAUAAGACC | | [2454-2472] | [2307-2325] |
| 209 | AUCCCAUCCAGUCCCGAAA | UUUCGGGACUGGAUGGGAU | | [673-691] | [673-691] |
| 210 | CUAGAAGGGCUCCAAACCA | UGGUUUGGAGCCCUUCUAG | | [178-196] | [178-196] |
| 211 | CAUUGCUGGUCUCACUGGA | UCCAGUGAGACCAGCAAUG | GP | [722-740] | [722-740] |
| 212 | CAUCUGGUACAAAUUCCAG | CUGGAAUUUGUACCAGAUG | | [1454-1472] | [1454-1472] |
| 213 | GUUUACUGUCACUUUUGAU | AUCAAAAGUGACAGUAAAC | | [2433-2451] | [2286-2304] |
| 214 | CAGAGAAGGAAGGCAGAAU | AUUCUGCCUUCCUUCUCUG | | [21-39] | [21-39] |
| 215 | UUGGUGGUUCAGAGUUAUA | UAUAACUCUGAACCACCAA | | [2196-2214] | [2049-2067] |
| 216 | GAUUUUCUUCUUCACUUGA | UCAAGUGAAGAAGAAAAUC | | [2511-2529] | [2364-2382] |
| 217 | CUUUGUAUCUUCAGUAAUU | AAUUACUGAAGAUACAAAG | | [1938-1956] | [1791-1809] |
| 218 | GUAGCAAGGCUUGAUAGCA | UGCUAUCAAGCCUUGCUAC | | [2172-2190] | [2025-2043] |
| 219 | GCAAGUGUGCAGAGUCUUU | AAAGACUCUGCACACUUGC | | [928-946] | [928-946] |
| 220 | ACAGCAAUUUAUUCUUAUU | AAUAAGAAUAAAUUGCUGU | | [2471-2489] | [2324-2342] |
| 221 | GCUUGAUAGCAGAGGUUUG | CAAACCUCUGCUAUCAAGC | | [2180-2198] | [2033-2051] |
| 222 | UCUGCUCUCUGCUUGAAUU | AAUUCAAGCAGAGAGCAGA | | [372-390] | [372-390] |
| 223 | UUGGGAGACCAAUGUGGGA | UCCCACAUUGGUCUCCCAA | Rat, Ms | [1720-1738] | [1573-1591] |
| 224 | GCUCCAGAGGAAGAUUUCU | AGAAAUCUUCCUCUGGAGC | | [1239-1257] | [1239-1257] |
| 225 | CAUCCGGAGGAGUUAUUUU | AAAAUAACUCCUCCGGAUG | | [791-809] | [791-809] |
| 226 | GAGAAGGCCGACAAAUACU | AGUAUUUGUCGGCCUUCUC | | [306-324] | [306-324] |
| 227 | AAUCCCAUCCAGUCCCGAA | UUCGGGACUGGAUGGGAUU | | [672-690] | [672-690] |
| 228 | CCAUGGAUCCUUUCUCAGA | UCUGAGAAAGGAUCCAUGG | | [2118-2136] | [1971-1989] |
| 229 | UUACCAAGGUUGUUAUGCA | UGCAUAACAACCUUGGUAA | | [1096-1114] | [1096-1114] |
| 230 | GAAAAUUUUUGAGUUAUAG | CUAUAACUCAAAAAUUUUC | | [1890-1908] | [1743-1761] |
| 231 | AGUUAUACAACUAAUCCCA | UGGGAUUAGUUGUAUAACU | | [2208-2226] | [2061-2079] |
| 232 | UCUCCAGCCUAUCUCAUGA | UCAUGAGAUAGGCUGGAGA | | [628-646] | [628-646] |
| 233 | UGUGCAGACCACAACCUCA | UGAGGUUGUGGUCUGCACA | | [1473-1491] | [1473-1491] |
| 234 | CACCGAUAUUCCAGUCUGG | CCAGACUGGAAUAUCGGUG | | [1839-1857] | [1692-1710] |
| 235 | UAUCUCAUGAUGAGAAAAA | UUUUUCUCAUCAUGAGAUA | | [637-655] | [637-655] |
| 236 | GAUAGCACAGUUCUGUCCA | UGGACAGAACUGUGCUAUC | | [4-22] | [4-22] |
| 237 | GGUCUGAAACAGAAAACCU | AGGUUUUCUGUUUCAGACC | Rat, GP | [1698-1716] | [1551-1569] |
| 238 | AGCCUAUCUCAUGAUGAGA | UCUCAUCAUGAGAUAGGCU | | [633-651] | [633-651] |
| 239 | UAGCCUUGAUUCUCAUGGU | ACCAUGAGAAUCAAGGCUA | Rb, GP | [754-772] | [754-772] |
| 240 | AAAUUUUUGAGUUAUAGGA | UCCUAUAACUCAAAAAUUU | | [1892-1910] | [1745-1763] |
| 241 | AAAGGAUUUUCUUCUUCAC | GUGAAGAAGAAAAUCCUUU | | [2507-2525] | [2360-2378] |
| 242 | UUGGAAUUGCAGAUGAACA | UGUUCAUCUGCAAUUCCAA | | [1128-1146] | [1128-1146] |
| 243 | UCAUAAGGGCUUUCGAACA | UGUUCGAAAGCCCUUAUGA | | [1300-1318] | [1300-1318] |
| 244 | CAUCAAAAAAUAAAGGAUU | AAUCCUUUAUUUUUUGAUG | | [2496-2514] | [2349-2367] |
| 245 | AAGCCUUGACUCCCUGGUA | UACCAGGGAGUCAAGGCUU | | [2010-2028] | [1863-1881] |
| 246 | CUUCAGCUACUGAGUUCAU | AUGAACUCAGUAGCUGAAG | | [775-793] | [775-793] |
| 247 | ACCUUUUUAUCUUCUAUAU | AUAUAGAAGAUAAAAAGGU | | [832-850] | [832-850] |
| 248 | GCUGAAUGUUUUCCUGUUU | AAACAGGAAAACAUUCAGC | | [266-284] | [266-284] |
| 249 | UUCCAGUCUGGAUCCUAGA | UCUAGGAUCCAGACUGGAA | | [1847-1865] | [1700-1718] |
| 250 | AUUUCUUCUCCAUUCAUAU | AUAUGAAUGGAGAAGAAAU | | [1252-1270] | [1252-1270] |
| 251 | CUGCUAUAGCAGAAGCCGA | UCGGCUUCUGCUAUAGCAG | | [578-596] | [578-596] |
| 252 | UAAAGGAUUUUCUUCUUCA | UGAAGAAGAAAAUCCUUUA | | [2506-2524] | [2359-2377] |
| 253 | CUGUAGGCGCCCUAAGUUU | AAACUUAGGGCGCCUACAG | | [974-992] | [974-992] |
| 254 | AGUCUUUUGAGAUGUGGGA | UCCCACAUCUCAAAAGACU | | [940-958] | [940-958] |
| 255 | GAGUUAUAGGAAUAAGGAC | GUCCUUAUUCCUAUAACUC | | [1900-1918] | [1753-1771] |
| 256 | UGGUAACUUCAGCUACUGA | UCAGUAGCUGAAGUUACCA | | [769-787] | [769-787] |
| 257 | CCCACAGAAGAUUUCUAAG | CUUAGAAAUCUUCUGUGGG | | [2295-2313] | [2148-2166] |
| 258 | UGAGCUUCAGCAACUUAAG | CUUAAGUUGCUGAAGCUCA | | [2061-2079] | [1914-1932] |
| 259 | UUCAAUUCUACUUCAACAA | UUGUUGAAGUAGAAUUGAA | Rat | [1870-1888] | [1723-1741] |
| 260 | CACAACCUCACCUUCCACA | UGUGGAAGGUGAGGUUGUG | | [492-510] | [492-510] |
| 261 | GUACAUGUUUACUGUCACU | AGUGACAGUAAACAUGUAC | | [2427-2445] | [2280-2298] |
| 262 | CAAAUGCUGUCACCGAUAU | AUAUCGGUGACAGCAUUUG | GP | [1829-1847] | [1682-1700] |
| 263 | UGAGAAGGCCGACAAAUAC | GUAUUUGUCGGCCUUCUCA | | [305-323] | [305-323] |
| 264 | AUAUAACACCAACAGUGUA | UACACUGUUGGUGUUAUAU | | [2411-2429] | [2264-2282] |
| 265 | AUGUGACAUUCACCAGCAU | AUGCUGGUGAAUGUCACAU | GP | [706-724] | [706-724] |
| 266 | GGAUUUUCUUCUUCACUUG | CAAGUGAAGAAGAAAAUCC | | [2510-2528] | [2363-2381] |
| 267 | UAGAAAGGUUCAAUUCUAC | GUAGAAUUGAACCUUUCUA | | [1862-1880] | [1715-1733] |
| 268 | AAGUCUUCUGGUAUACUCA | UGAGUAUACCAGAAGACUU | | [811-829] | [811-829] |
| 269 | AGAAAGGUUCAAUUCUACU | AGUAGAAUUGAACCUUUCU | | [1863-1881] | [1716-1734] |
| 270 | ACUGAGAAAGCAAUUGGAU | AUCCAAUUGCUUUCUCAGU | | [473-491] | [473-491] |
| 271 | UGAGAAAGCAAUUGGAUCA | UGAUCCAAUUGCUUUCUCA | | [475-493] | [475-493] |
| 272 | AGGAUUUUCUUCUUCACUU | AAGUGAAGAAGAAAAUCCU | | [2509-2527] | [2362-2380] |
| 273 | UUUCGAACAACAAUAUUCA | UGAAUAUUGUUGUUCGAAA | | [1310-1328] | [1310-1328] |
| 274 | ACAGAAGAUUUCUAAGUAG | CUACUUAGAAAUCUUCUGU | Rat | [2298-2316] | [2151-2169] |
| 275 | UGAGAUGUGGGAUGAUCGU | ACGAUCAUCCCACAUCUCA | | [947-965] | [947-965] |
| 276 | AUCUCAUGAUGAGAAAAAG | CUUUUUCUCAUCAUGAGAU | | [638-656] | [638-656] |
| 277 | GUAUCUUCAGUAAUUUACU | AGUAAAUUACUGAAGAUAC | | [1942-1960] | [1795-1813] |
| 278 | AUCCUUUCUCAGAAAAAUA | UAUUUUUCUGAGAAAGGAU | | [2124-2142] | [1977-1995] |
| 279 | CAUAUAACACCAACAGUGU | ACACUGUUGGUGUUAUAUG | | [2410-2428] | [2263-2281] |
| 280 | UAUAUGAUCUGCCUACAUA | UAUGUAGGCAGAUCAUAUA | | [522-540] | [522-540] |
| 281 | GUCUCGUCAGGUUUGAGCA | UGCUCAAACCUGACGAGAC | | [1963-1981] | [1816-1834] |
| 282 | ACAACAAUAUUCACCAAUU | AAUUGGUGAAUAUUGUUGU | | [1316-1334] | [1316-1334] |
| 283 | ACUGGUUUUCAGUUUUGUU | AACAAAACUGAAAACCAGU | | [229-247] | [229-247] |
| 284 | CAUUCAACUUCGUUACUUG | CAAGUAACGAAGUUGAAUG | | [2044-2062] | [1897-1915] |
| 285 | GGAGUGAUCAUGACAAUAG | CUAUUGUCAUGAUCACUCC | | [738-756] | [738-756] |
| 286 | GUUUGAGCAGUCACUUUAG | CUAAAGUGACUGCUCAAAC | | [1973-1991] | [1826-1844] |
| 287 | GUUGUUAUGCACCCAUCCA | UGGAUGGGUGCAUAACAAC | | [1104-1122] | [1104-1122] |
| 288 | CAAUCUCUCUCCUGGAAUG | CAUUCCAGGAGAGAGAUUG | | [1198-1216] | [1198-1216] |
| 289 | UUGUGAUUACCAAGGUUGU | ACAACCUUGGUAAUCACAA | | [1090-1108] | [1090-1108] |
| 290 | AAUCCGGAUUGCUGAAUCU | AGAUUCAGCAAUCCGGAUU | | [149-167] | [149-167] |
| 291 | ACACCAACAGUGUACAUGU | ACAUGUACACUGUUGGUGU | | [2416-2434] | [2269-2287] |
| 292 | GAGAGUCAUCCUCGCAAGU | ACUUGCGAGGAUGACUCUC | | [915-933] | [915-933] |
| 293 | GUUCUUAAAGCCCAUGGAU | AUCCAUGGGCUUUAAGAAC | | [2107-2125] | [1960-1978] |
| 294 | CAAAGUUUUGGAAUUGCAG | CUGCAAUUCCAAAACUUUG | | [1121-1139] | [1121-1139] |
| 295 | CUUCUCCAUUCAUAUCCGA | UCGGAUAUGAAUGGAGAAG | | [1256-1274] | [1256-1274] |
| 296 | UGCUGGUUGGAGCAGGAAU | AUUCCUGCUCCAACCAGCA | | [1402-1420] | [1402-1420] |
| 297 | AACAGCAAUUUAUUCUUAU | AUAAGAAUAAAUUGCUGUU | | [2470-2488] | [2323-2341] |
| 298 | CUACAAUAGCUACCUCCCA | UGGGAGGUAGCUAUUGUAG | | [1750-1768] | [1603-1621] |
| 299 | AAAUGCUGUCACCGAUAUU | AAUAUCGGUGACAGCAUUU | GP | [1830-1848] | [1683-1701] |
| 300 | CUUGCACCGGUCAUUCUUU | AAAGAAUGACCGGUGCAAG | | [1026-1044] | [1026-1044] |
| 301 | CCAGUGUGAACAGCAAUUU | AAAUUGCUGUUCACACUGG | | [2462-2480] | [2315-2333] |
| 302 | UAAUAAACAAAAUUACCGA | UCGGUAAUUUUGUUUAUUA | | [2379-2397] | [2232-2250] |
| 303 | UUUUGAGUUAUAGGAAUAA | UUAUUCCUAUAACUCAAAA | | [1896-1914] | [1749-1767] |
| 304 | UUGAAAUCCAUCUGGUACA | UGUACCAGAUGGAUUUCAA | | [1446-1464] | [1446-1464] |
| 305 | UGACAAUGGGAAACUGGGU | ACCCAGUUUCCCAUUGUCA | | [202-220] ORF | [202-220] ORF |
| 306 | CAGAAAAUCUCAUAAGGGC | GCCCUUAUGAGAUUUUCUG | | [1291-1309] | [1291-1309] |
| 307 | UCACGGCAUUGGUGGAAUU | AAUUCCACCAAUGCCGUGA | | [866-884] | [866-884] |
| 308 | CAACAAUAUUCACCAAUUC | GAAUUGGUGAAUAUUGUUG | | [1317-1335] | [1317-1335] |
| 309 | AAGGCUUGAUAGCAGAGGU | ACCUCUGCUAUCAAGCCUU | | [2177-2195] | [2030-2048] |
| 310 | AUUCAACUUCGUUACUUGA | UCAAGUAACGAAGUUGAAU | | [2045-2063] | [1898-1916] |
| 311 | ACUUCAACAAAGAAAAUUU | AAAUUUUCUUUGUUGAAGU | | [1879-1897] | [1732-1750] |
| 312 | AAAGUUUUGGAAUUGCAGA | UCUGCAAUUCCAAAACUUU | Rat | [1122-1140] | [1122-1140] |
| 313 | AGAGGAGAGCAUGAAUGAG | CUCAUUCAUGCUCUCCUCU | | [899-917] | [899-917] |
| 314 | GAAGAUUUCUAAGUAGGGU | ACCCUACUUAGAAAUCUUC | Rat | [2301-2319] | [2154-2172] |
| 315 | CUUUAUAUCUGUGAAAGGA | UCCUUUCACAGAUAUAAAG | | [1041-1059] | [1041-1059] |
| 316 | AUGGUCUUAUCCAGUGUGA | UCACACUGGAUAAGACCAU | | [2452-2470] | [2305-2323] |
| 317 | UAUUCUUAUUUUUGCUCAU | AUGAGCAAAAAUAAGAAUA | | [2480-2498] | [2333-2351] |
| 318 | GCAGUCACUUUAGGAUAAG | CUUAUCCUAAAGUGACUGC | | [1979-1997] | [1832-1850] |
| 319 | UUUGACUGCUAUAGCAGAA | UUCUGCUAUAGCAGUCAAA | | [573-591] | [573-591] |
| 320 | AUUCUUUUUUUGAUUGCAU | AUGCAAUCAAAAAAAGAAU | | [2028-2046] | [1881-1899] |
| 321 | GAAAGGUUCAAUUCUACUU | AAGUAGAAUUGAACCUUUC | | [1864-1882] | [1717-1735] |
| 322 | ACCAAUGUGGGACAAUGAG | CUCAUUGUCCCACAUUGGU | Rat, Ms | [1727-1745] | [1580-1598] |
| 323 | AGUUUUGGUUUGUAAUCUU | AAGAUUACAAACCAAAACU | | [2262-2280] | [2115-2133] |
| 324 | GAUAAGAAUGUGCCUCUCA | UGAGAGGCACAUUCUUAUC | | [1992-2010] | [1845-1863] |
| 325 | AAUAUGAGAAGGCCGACAA | UUGUCGGCCUUCUCAUAUU | | [301-319] | [301-319] |
| 326 | CAUACAGCUAUUCACAUCA | UGAUGUGAAUAGCUGUAUG | | [537-555] | [537-555] |
| 327 | AUCCCAGGUGAUUUUAUCA | UGAUAAAAUCACCUGGGAU | | [2221-2239] | [2074-2092] |
| 328 | AUAAGGACGGUAAUCUGCA | UGCAGAUUACCGUCCUUAU | | [1911-1929] | [1764-1782] |
| 329 | GAUGUUUUCCAGUAUGAAG | CUUCAUACUGGAAAACAUC | Ms | [1377-1395] | [1377-1395] |
| 330 | UAUGAUCUGCCUACAUACA | UGUAUGUAGGCAGAUCAUA | | [524-542] | [524-542] |
| 331 | CAAUUCCCAGGAUUGAAGU | ACUUCAAUCCUGGGAAUUG | | [1330-1348] | [1330-1348] |
| 332 | UUCUACUUCAACAAAGAAA | UUUCUUUGUUGAAGUAGAA | Rat, Ms | [1875-1893] | [1728-1746] |
| 333 | UGAUAUGGUCUUAUCCAGU | ACUGGAUAAGACCAUAUCA | | [2448-2466] | [2301-2319] |
| 334 | UCAUCAAAAAAUAAAGGAU | AUCCUUUAUUUUUUGAUGA | | [2495-2513] | [2348-2366] |
| 335 | UUUGGUGGUUCAGAGUUAU | AUAACUCUGAACCACCAAA | | [2195-2213] | [2048-2066] |
| 336 | ACCGAUAUUCCAGUCUGGA | UCCAGACUGGAAUAUCGGU | | [1840-1858] | [1693-1711] |
| 337 | UUGUAUCUUCAGUAAUUUA | UAAAUUACUGAAGAUACAA | | [1940-1958] | [1793-1811] |
| 338 | CUCAUUUUGCAGCCGCACA | UGUGCGGCUGCAAAAUGAG | | [455-473] | [455-473] |
| 339 | CUUGAGCUUCAGCAACUUA | UAAGUUGCUGAAGCUCAAG | | [2059-2077] | [1912-1930] |
| 340 | AUCAUUGCACACCUGUUUA | UAAACAGGUGUGCAAUGAU | | [552-570] | [552-570] |
| 341 | GUGAUUACCAAGGUUGUUA | UAACAACCUUGGUAAUCAC | | [1092-1110] | [1092-1110] |
| 342 | CUCAUCAAAAAAUAAAGGA | UCCUUUAUUUUUUGAUGAG | | [2494-2512] | [2347-2365] |
| 343 | AAGUUCUUAAAGUUCUGAA | UUCAGAACUUUAAGAACUU | | [2088-2106] | [1941-1959] |
| 344 | UUCUUUUUUUGAUUGCAUU | AAUGCAAUCAAAAAAAGAA | | [2029-2047] | [1882-1900] |
| 345 | AAGAAAAAUCCUUGGGUCA | UGACCCAAGGAUUUUUCUU | | [332-350] | [332-350] |
| 346 | GAAUGAGAGUCAUCCUCGC | GCGAGGAUGACUCUCAUUC | | [911-929] | [911-929] |
| 347 | AUUUUUGAGUUAUAGGAAU | AUUCCUAUAACUCAAAAAU | | [1894-1912] | [1747-1765] |
| 348 | AGUGUGCAGAGUCUUUUGA | UCAAAAGACUCUGCACACU | | [931-949] | [931-949] |
| 349 | UUCUCUCCAGCCUAUCUCA | UGAGAUAGGCUGGAGAGAA | Ms | [625-643] | [625-643] |
| 350 | CAGCAAUUUAUUCUUAUUU | AAAUAAGAAUAAAUUGCUG | | [2472-2490] | [2325-2343] |
| 351 | AGAAAGCAAUUGGAUCACA | UGUGAUCCAAUUGCUUUCU | | [477-495] | [477-495] |
| 352 | UUCUAAGUAGGGUGACUUU | AAAGUCACCCUACUUAGAA | | [2307-2325] | [2160-2178] |
| 353 | UUUACCGCUCCCAGCAGAA | UUCUGCUGGGAGCGGUAAA | | [1069-1087] | [1069-1087] |
| 354 | UUUAACUUUGACUGCUAUA | UAUAGCAGUCAAAGUUAAA | | [567-585] | [567-585] |
| 355 | UGUGAUUACCAAGGUUGUU | AACAACCUUGGUAAUCACA | | [1091-1109] | [1091-1109] |
| 356 | AGGUUUGGUGGUUCAGAGU | ACUCUGAACCACCAAACCU | | [2192-2210] | [2045-2063] |
| 357 | CAGAGGUUUGGUGGUUCAG | CUGAACCACCAAACCUCUG | | [2189-2207] | [2042-2060] |
| 358 | ACAAUGAGUUUUCUACAAU | AUUGUAGAAAACUCAUUGU | | [1738-1756] | [1591-1609] |
| 359 | GAUGUGGGAUGAUCGUGAC | GUCACGAUCAUCCCACAUC | | [950-968] | [950-968] |
| 360 | UUCACAUCUUAAAGGCUCA | UGAGCCUUUAAGAUGUGAA | | [83-101] | [83-101] |
| 361 | AAUUCCAGUGUGCAGACCA | UGGUCUGCACACUGGAAUU | | [1465-1483] | [1465-1483] |
| 362 | AAUAAAGGAUUUUCUUCUU | AAGAAGAAAAUCCUUUAUU | | [2504-2522] | [2357-2375] |
| 363 | UAACACCAACAGUGUACAU | AUGUACACUGUUGGUGUUA | | [2414-2432] | [2267-2285] |
| 364 | AUGAGAGUCAUCCUCGCAA | UUGCGAGGAUGACUCUCAU | | [913-931] | [913-931] |
| 365 | UAAUCCCAGGUGAUUUUAU | AUAAAAUCACCUGGGAUUA | | [2219-2237] | [2072-2090] |
| 366 | UAGCAAGGCUUGAUAGCAG | CUGCUAUCAAGCCUUGCUA | | [2173-2191] | [2026-2044] |
| 367 | GCAAUCUGCUGUCCUUCCU | AGGAAGGACAGCAGAUUGC | | [424-442] | [424-442] |
| 368 | CCACAGAAGAUUUCUAAGU | ACUUAGAAAUCUUCUGUGG | | [2296-2314] | [2149-2167] |
| 369 | AGAUUUCUAAGUAGGGUGA | UCACCCUACUUAGAAAUCU | Rat | [2303-2321] | [2156-2174] |
| 370 | GUGUACAUGUUUACUGUCA | UGACAGUAAACAUGUACAC | | [2425-2443] | [2278-2296] |
| 371 | AUUCCAGUGUGCAGACCAC | GUGGUCUGCACACUGGAAU | | [1466-1484] | [1466-1484] |
| 372 | UUUUUGAGUUAUAGGAAUA | UAUUCCUAUAACUCAAAAA | | [1895-1913] | [1748-1766] |
| 373 | ACUGUAGGCGCCCUAAGUU | AACUUAGGGCGCCUACAGU | | [973-991] | [973-991] |
| 374 | UUCUGAAGUUCUUAAAGUU | AACUUUAAGAACUUCAGAA | | [2083-2101] | [1936-1954] |
| 375 | GAUUGCAUUCAACUUCGUU | AACGAAGUUGAAUGCAAUC | | [2039-2057] | [1892-1910] |
| 376 | AAAGCAAUUGGAUCACAAC | GUUGUGAUCCAAUUGCUUU | | [479-497] | [479-497] |
| 377 | AUCCAGUGUGAACAGCAAU | AUUGCUGUUCACACUGGAU | | [2460-2478] | [2313-2331] |
| 378 | UUGAUAUGGUCUUAUCCAG | CUGGAUAAGACCAUAUCAA | | [2447-2465] | [2300-2318] |
| 379 | AGGCGCCCUAAGUUUGAAG | CUUCAAACUUAGGGCGCCU | | [978-996] | [978-996] |
| 380 | CAAAUUCCAGUGUGCAGAC | GUCUGCACACUGGAAUUUG | | [1463-1481] | [1463-1481] |
| 381 | CACUGACAUCGUGACAGGU | ACCUGUCACGAUGUCAGUG | | [1682-1700] | [1535-1553] |
| 382 | UUUUGCAGCCGCACACUGA | UCAGUGUGCGGCUGCAAAA | | [459-477] | [459-477] |
| 383 | UAAGAAUGUGCCUCUCAAG | CUUGAGAGGCACAUUCUUA | | [1994-2012] | [1847-1865] |
| 384 | GGUCUCACUGGAGUGAUCA | UGAUCACUCCAGUGAGACC | | [729-747] | [729-747] |
| 385 | UAUACUCACCACCUUUUUA | UAAAAAGGUGGUGAGUAUA | | [822-840] | [822-840] |
| 386 | GUGUGCAGAGUCUUUUGAG | CUCAAAAGACUCUGCACAC | | [932-950] | [932-950] |
| 387 | AUUGCAUUCAACUUCGUUA | UAACGAAGUUGAAUGCAAU | | [2040-2058] | [1893-1911] |
| 388 | AAUCUGCUGUCCUUCCUGA | UCAGGAAGGACAGCAGAUU | | [426-444] | [426-444] |
| 389 | UAUUCUUUUUUUGAUUGCA | UGCAAUCAAAAAAAGAAUA | | [2027-2045] | [1880-1898] |
| 390 | AAAAAUCCUUGGGUCAACA | UGUUGACCCAAGGAUUUUU | | [335-353] | [335-353] |
| 391 | GCUAUAGCAGAAGCCGACA | UGUCGGCUUCUGCUAUAGC | | [580-598] | [580-598] |
| 392 | AUACAGCUAUUCACAUCAU | AUGAUGUGAAUAGCUGUAU | | [538-556] | [538-556] |
| 393 | UUGCACACCUGUUUAACUU | AAGUUAAACAGGUGUGCAA | | [556-574] | [556-574] |
| 394 | GUAACUUCAGCUACUGAGU | ACUCAGUAGCUGAAGUUAC | | [771-789] | [771-789] |
| 395 | UAAAGUUCUGAAGUUCUUA | UAAGAACUUCAGAACUUUA | | [2095-2113] | [1948-1966] |
| 396 | UUAGGAUAAGAAUGUGCCU | AGGCACAUUCUUAUCCUAA | | [1988-2006] | [1841-1859] |
| 397 | UUUGAUAUGGUCUUAUCCA | UGGAUAAGACCAUAUCAAA | | [2446-2464] | [2299-2317] |
| 398 | CAUUCUCUCCAGCCUAUCU | AGAUAGGCUGGAGAGAAUG | | [623-641] | [623-641] |
| 399 | UCCAUUCUCUCCAGCCUAU | AUAGGCUGGAGAGAAUGGA | | [621-639] | [621-639] |
| 400 | ACCUCUCCAGAAUCCGGAU | AUCCGGAUUCUGGAGAGGU | | [139-157] | [139-157] |
| 401 | UAGCACAGUUCUGUCCAGA | UCUGGACAGAACUGUGCUA | | [6-24] | [6-24] |
| 402 | GAUGCCUUCCUGAAAUAUG | CAUAUUUCAGGAAGGCAUC | | [288-306] | [288-306] |
| 403 | UUUUGAUUGCAUUCAACUU | AAGUUGAAUGCAAUCAAAA | | [2035-2053] | [1888-1906] |
| 404 | GUUCAUCCGGAGGAGUUAU | AUAACUCCUCCGGAUGAAC | | [788-806] | [788-806] |
| 405 | UUGAGCAGUCACUUUAGGA | UCCUAAAGUGACUGCUCAA | | [1975-1993] | [1828-1846] |
| 406 | UGUUUUCCUGUUUGUGGAU | AUCCACAAACAGGAAAACA | | [272-290] | [272-290] |
| 407 | UUCCAGUGUGCAGACCACA | UGUGGUCUGCACACUGGAA | | [1467-1485] | [1467-1485] |
| 408 | CAUUGCACACCUGUUUAAC | GUUAAACAGGUGUGCAAUG | | [554-572] | [554-572] |
| 409 | AUGUUCCAUUCCUGAAGGA | UCCUUCAGGAAUGGAACAU | | [121-139] | [121-139] |
| 410 | CUUCCCUGUUGCCUAGAAG | CUUCUAGGCAACAGGGAAG | | [166-184] | [166-184] |
| 411 | UUGUCUCUUUGUAUCUUCA | UGAAGAUACAAAGAGACAA | | [1932-1950] | [1785-1803] |
| 412 | UGUCCAGAGAAGGAAGGCA | UGCCUUCCUUCUCUGGACA | | [17-35] | [17-35] |
| 413 | UGACUGUAGCAAGGCUUGA | UCAAGCCUUGCUACAGUCA | | [2167-2185] | [2020-2038] |
| 414 | UACAACUAAUCCCAGGUGA | UCACCUGGGAUUAGUUGUA | | [2213-2231] | [2066-2084] |
| 415 | GCAUUCAACUUCGUUACUU | AAGUAACGAAGUUGAAUGC | | [2043-2061] | [1896-1914] |
| 416 | UGAUUUUAUCAAUUCCAGU | ACUGGAAUUGAUAAAAUCA | | [2229-2247] | [2082-2100] |
| 417 | GGAGUAUGUGACAUUCACC | GGUGAAUGUCACAUACUCC | | [701-719] | [701-719] |
| 418 | UGAACAGCAAUUUAUUCUU | AAGAAUAAAUUGCUGUUCA | | [2468-2486] | [2321-2339] |
| 419 | CUUCCUGAAAUAUGAGAAG | CUUCUCAUAUUUCAGGAAG | | [293-311] | [293-311] |
| 420 | UACUUUGACCUCUGCUCCA | UGGAGCAGAGGUCAAAGUA | GP | [1226-1244] | [1226-1244] |
| 421 | AGUCCCGAAACACGACAGU | ACUGUCGUGUUUCGGGACU | | [682-700] | [682-700] |
| 422 | AAUGUGGGACAAUGAGUUU | AAACUCAUUGUCCCACAUU | Rat, Ms | [1730-1748] | [1583-1601] |
| 423 | GUUACCAUCUCCUGAGUUU | AAACUCAGGAGAUGGUAAC | | [2248-2266] | [2101-2119] |
| 424 | GGAAUAAGGACGGUAAUCU | AGAUUACCGUCCUUAUUCC | | [1908-1926] | [1761-1779] |
| 425 | GAAGGCCGACAAAUACUAC | GUAGUAUUUGUCGGCCUUC | | [308-326] | [308-326] |
| 426 | AUUGCACACCUGUUUAACU | AGUUAAACAGGUGUGCAAU | | [555-573] | [555-573] |
| 427 | AUAUGAUCUGCCUACAUAC | GUAUGUAGGCAGAUCAUAU | | [523-541] | [523-541] |
| 428 | UUCUUAUUUUUGCUCAUCA | UGAUGAGCAAAAAUAAGAA | | [2482-2500] | [2335-2353] |
| 429 | CAGAAGAUUUCUAAGUAGG | CCUACUUAGAAAUCUUCUG | Rat | [2299-2317] | [2152-2170] |
| 430 | GAGUUCAUCCGGAGGAGUU | AACUCCUCCGGAUGAACUC | | [786-804] | [786-804] |
| 431 | UCUCAGAAAAAUAACUGUA | UACAGUUAUUUUUCUGAGA | | [2130-2148] | [1983-2001] |
| 432 | AAGUGUGCAGAGUCUUUUG | CAAAAGACUCUGCACACUU | | [930-948] | [930-948] |
| 433 | AAGAGCCUGCGCAAAUGCU | AGCAUUUGCGCAGGCUCUU | | [1818-1836] | [1671-1689] |
| 434 | CUGAUAGCACAGUUCUGUC | GACAGAACUGUGCUAUCAG | | [2-20] | [2-20] |
| 435 | GAAGGACCUCUCCAGAAUC | GAUUCUGGAGAGGUCCUUC | | [134-152] | [134-152] |
| 436 | AUAGCACAGUUCUGUCCAG | CUGGACAGAACUGUGCUAU | | [5-23] | [5-23] |
| 437 | UUUGGUUAGGGCUGAAUGU | ACAUUCAGCCCUAACCAAA | | [256-274] | [256-274] |
| 438 | AGCACAGUUCUGUCCAGAG | CUCUGGACAGAACUGUGCU | | [7-25] | [7-25] |
| 439 | CCUUUCUCAGAAAAAUAAC | GUUAUUUUUCUGAGAAAGG | | [2126-2144] | [1979-1997] |
| 440 | GUCUGUAGUGGGAGUUUUC | GAAAACUCCCACUACAGAC | | [1775-1793] | [1628-1646] |
| 441 | CUUUUGAGAUGUGGGAUGA | UCAUCCCACAUCUCAAAAG | | [943-961] | [943-961] |
| 442 | AAGGAUUUUCUUCUUCACU | AGUGAAGAAGAAAAUCCUU | | [2508-2526] | [2361-2379] |
| 443 | CACAUCAUUGCACACCUGU | ACAGGUGUGCAAUGAUGUG | | [549-567] | [549-567] |
| 444 | GCUCAUCAAAAAAUAAAGG | CCUUUAUUUUUUGAUGAGC | | [2493-2511] | [2346-2364] |
| 445 | GUUUGUGGAUGCCUUCCUG | CAGGAAGGCAUCCACAAAC | | [281-299] | [281-299] |
| 446 | UUGCCUAGAAGGGCUCCAA | UUGGAGCCCUUCUAGGCAA | | [174-192] | [174-192] |
| 447 | AGUAUAUCUUUGUUAAUUG | CAAUUAACAAAGAUAUACU | | [1174-1192] | [1174-1192] |
| 448 | AUUUUAACAGCACGCUGAU | AUCAGCGUGCUGUUAAAAU | | [388-406] | [388-406] |
| 449 | UGCCUUCCUGAAAUAUGAG | CUCAUAUUUCAGGAAGGCA | | [290-308] | [290-308] |
| 450 | ACGGCAUUGGUGGAAUUGU | ACAAUUCCACCAAUGCCGU | | [868-886] | [868-886] |
| 451 | CAGCUAUUCACAUCAUUGC | GCAAUGAUGUGAAUAGCUG | | [541-559] | [541-559] |
| 452 | GUUGUUUGGUUAGGGCUGA | UCAGCCCUAACCAAACAAC | | [252-270] | [252-270] |
| 453 | AAUGAGUUUUCUACAAUAG | CUAUUGUAGAAAACUCAUU | | [1740-1758] | [1593-1611] |
| 454 | CAUUAAACUUUGACAAGGC | GCCUUGUCAAAGUUUAAUG | | [1663-1681] | [1516-1534] |
| 455 | GGUAAUCUGCAUUUUGUCU | AGACAAAAUGCAGAUUACC | | [1919-1937] | [1772-1790] |
| 456 | CUGUAGCAAGGCUUGAUAG | CUAUCAAGCCUUGCUACAG | | [2170-2188] | [2023-2041] |
| 457 | UAUGUGACAUUCACCAGCA | UGCUGGUGAAUGUCACAUA | GP | [705-723] | [705-723] |
| 458 | UUUUGAGAUGUGGGAUGAU | AUCAUCCCACAUCUCAAAA | | [944-962] | [944-962] |
| 459 | AAUCCCAGGUGAUUUUAUC | GAUAAAAUCACCUGGGAUU | | [2220-2238] | [2073-2091] |
| 460 | UACAUACAGCUAUUCACAU | AUGUGAAUAGCUGUAUGUA | | [535-553] | [535-553] |
| 461 | CGGAUUGCUGAAUCUUCCC | GGGAAGAUUCAGCAAUCCG | | [153-171] | [153-171] |
| 462 | AAUGAGAGUCAUCCUCGCA | UGCGAGGAUGACUCUCAUU | | [912-930] | [912-930] |
| 463 | UUUUUUGAUUGCAUUCAAC | GUUGAAUGCAAUCAAAAAA | | [2033-2051] | [1886-1904] |
| 464 | AGAAGAUUUCUAAGUAGGG | CCCUACUUAGAAAUCUUCU | Rat | [2300-2318] | [2153-2171] |
| 465 | UAUGAGAAGGCCGACAAAU | AUUUGUCGGCCUUCUCAUA | | [303-321] | [303-321] |
| 466 | UUAAAGUUCUGAAGUUCUU | AAGAACUUCAGAACUUUAA | | [2094-2112] | [1947-1965] |
| 467 | GACAUCGUGACAGGUCUGA | UCAGACCUGUCACGAUGUC | | [1686-1704] | [1539-1557] |
| 468 | GAAAACCUCCUUUGGGAGA | UCUCCCAAAGGAGGUUUUC | Rat | [1709-1727] | [1562-1580] |
| 469 | UUUGCUUCUAUCUUGAAAU | AUUUCAAGAUAGAAGCAAA | | [1434-1452] | [1434-1452] |
| 470 | UUUACUGUCACUUUUGAUA | UAUCAAAAGUGACAGUAAA | | [2434-2452] | [2287-2305] |
| 471 | GCACACCUGUUUAACUUUG | CAAAGUUAAACAGGUGUGC | Rb | [558-576] | [558-576] |
| 472 | CAAUCUGCUGUCCUUCCUG | CAGGAAGGACAGCAGAUUG | | [425-443] | [425-443] |
| 473 | CAUUUUGCAGCCGCACACU | AGUGUGCGGCUGCAAAAUG | | [457-475] | [457-475] |
| 474 | AUUGCUGAAUCUUCCCUGU | ACAGGGAAGAUUCAGCAAU | | [156-174] | [156-174] |
| 475 | UUGGUCUCGUCAGGUUUGA | UCAAACCUGACGAGACCAA | | [1960-1978] | [1813-1831] |
| 476 | UUCCAUUCCUGAAGGACCU | AGGUCCUUCAGGAAUGGAA | | [124-142] | [124-142] |
| 477 | GAUUGAAGUGGAUGGUCCC | GGGACCAUCCACUUCAAUC | | [1340-1358] | [1340-1358] |
| 478 | AUCUUGAAAUCCAUCUGGU | ACCAGAUGGAUUUCAAGAU | GP | [1443-1461] | [1443-1461] |
| 479 | CCUUUGGGAGACCAAUGUG | CACAUUGGUCUCCCAAAGG | Rat, Ms | [1717-1735] | [1570-1588] |
| 480 | GUUUUGGUUUGUAAUCUUU | AAAGAUUACAAACCAAAAC | | [2263-2281] | [2116-2134] |
| 481 | GUUAACCACUGGUUUUCAG | CUGAAAACCAGUGGUUAAC | | [222-240] | [222-240] |
| 482 | CUGUCACCGAUAUUCCAGU | ACUGGAAUAUCGGUGACAG | GP | [1835-1853] | [1688-1706] |
| 483 | AACUAAUCCCAGGUGAUUU | AAAUCACCUGGGAUUAGUU | | [2216-2234] | [2069-2087] |
| 484 | AUUCCAGUCUGGAUCCUAG | CUAGGAUCCAGACUGGAAU | | [1846-1864] | [1699-1717] |
| 485 | GAUGAUCGUGACUCCCACU | AGUGGGAGUCACGAUCAUC | | [957-975] | [957-975] |
| 486 | CUCCUUUGGGAGACCAAUG | CAUUGGUCUCCCAAAGGAG | Rat | [1715-1733] | [1568-1586] |
| 487 | UAGUGGGAGUUUUCUUAUG | CAUAAGAAAACUCCCACUA | | [1780-1798] | [1633-1651] |
| 488 | AAUUUUAACAGCACGCUGA | UCAGCGUGCUGUUAAAAUU | | [387-405] | [387-405] |
| 489 | UGUUUUUCACAUCUUAAAG | CUUUAAGAUGUGAAAAACA | | [78-96] | [78-96] |
| 490 | CUUUCUCAGAAAAAUAACU | AGUUAUUUUUCUGAGAAAG | | [2127-2145] | [1980-1998] |
| 491 | AUCUGGUACAAAUUCCAGU | ACUGGAAUUUGUACCAGAU | | [1455-1473] | [1455-1473] |
| 492 | GACAUUCACCAGCAUUGCU | AGCAAUGCUGGUGAAUGUC | GP | [710-728] | [710-728] |
| 493 | AUUUUGAAGUCUUCUGGUA | UACCAGAAGACUUCAAAAU | | [805-823] | [805-823] |
| 494 | UAAUCUUUUGUCCCUCCCA | UGGGAGGGACAAAAGAUUA | | [2274-2292] | [2127-2145] |
| 495 | CUUGAUUCUCAUGGUAACU | AGUUACCAUGAGAAUCAAG | Rb, GP | [758-776] | [758-776] |
| 496 | AGUGAGGAUGUUUUCCAGU | ACUGGAAAACAUCCUCACU | Ms | [1371-1389] | [1371-1389] |
| 497 | GGUUGUUAUGCACCCAUCC | GGAUGGGUGCAUAACAACC | | [1103-1121] | [1103-1121] |
| 498 | ACUGGGUGGUUAACCACUG | CAGUGGUUAACCACCCAGU | | [214-232] | [214-232] |
| 499 | UCACAACCUCACCUUCCAC | GUGGAAGGUGAGGUUGUGA | | [491-509] | [491-509] |
| 500 | GAGUCUUUUGAGAUGUGGG | CCCACAUCUCAAAAGACUC | | [939-957] | [939-957] |

TABLE A5
CYBB 19-mer oligomers NOX2 (NADPH oxidase 2; CYBB)

| | | | | |
|---|---|---|---|---|
| | | | | Human-6996020 |
| No. | Sense siRNA | AntiSense siRNA | Other Sp | ORF: 15-1727 |

| | | | | |
|---|---|---|---|---|
| 1 | GGGUUUUCUUUUUGUUCUU | AAGAACAAAAAGAAAACCC | | [3055-3073] |
| 2 | CCAGAUCAUUAGGACAUAA | UUAUGUCCUAAUGAUCUGG | | [2240-2258] |
| 3 | GCCUCAUAUUAAUUAUCAC | GUGAUAAUUAAUAUGAGGC | | [568-586] |
| 4 | GCAUAAUUAAAACCAGCUU | AAGCUGGUUUUAAUUAUGC | | [3800-3818] |
| 5 | GCAGGGUUUUCUUUUUGUU | AACAAAAAGAAAACCCUGC | | [3052-3070] |
| 6 | CCAACUGGGAUAAUGAAUU | AAUUCAUUAUCCCAGUUGG | | [1555-1573] |
| 7 | GGGUCUCAUAUUGGUCAUU | AAUGACCAAUAUGAGACCC | | [3291-3309] |
| 8 | GGAUAAUGAAUUCAAGACA | UGUCUUGAAUUCAUUAUCC | | [1562-1580] |
| 9 | GCCUGAAUUUCAACUGCAU | AUGCAGUUGAAAUUCAGGC | Rat | [190-208] |
| 10 | CGCUGUGCCUCAUAUUAAU | AUUAAUAUGAGGCACAGCG | | [562-580] |
| 11 | CUAACCUAUGUGCAUAAUU | AAUUAUGCACAUAGGUUAG | | [3789-3807] |
| 12 | CAAUUCAUCUAUUCAACAG | CUGUUGAAUAGAUGAAUUG | | [3653-3671] |
| 13 | GUGUGACUAAUGGUUUAUU | AAUAAACCAUUAGUCACAC | | [2892-2910] |
| 14 | ACCACACAAUGCCAAGAUA | UAUCUUGGCAUUGUGUGGU | | [2473-2491] |
| 15 | GCCUCAGAAUCCUCAAAAU | AUUUUGAGGAUUCUGAGGC | | [2118-2136] |
| 16 | GAAACCCUGAGUAAACAAA | UUUGUUUACUCAGGGUUUC | | [1644-1662] |
| 17 | CCAACUAAGUUAUGAAAGU | ACUUUCAUAACUUAGUUGG | | [3866-3884] |
| 18 | CCAUUGCACAUCUAUUUAA | UUAAAUAGAUGUGCAAUGG | | [361-379] |
| 19 | CAGGUAUCAAUUCAUCUAU | AUAGAUGAAUUGAUACCUG | | [3646-3664] |
| 20 | CGAAUAUAGCCUUUCUGAA | UUCAGAAAGGCUAUAUUCG | | [3568-3586] |
| 21 | GGCCUGCUGUGCGAAUAUA | UAUAUUCGCACAGCAGGCC | | [3557-3575] |
| 22 | AGGGUUUUCUUUUUGUUCU | AGAACAAAAAGAAAACCCU | | [3054-3072] |
| 23 | CCCUUAUGAAUAUUGAACU | AGUUCAAUAUUCAUAAGGG | | [2775-2793] |
| 24 | CUAAUUUCCUGCCCAAAGU | ACUUUGGGCAGGAAAUUAG | | [2740-2758] |
| 25 | CAACACCCUAAUACCAGAA | UUCUGGUAUUAGGGUGUUG | Rb | [1590-1608] |
| 26 | GGGAUAAUGAAUUCAAGAC | GUCUUGAAUUCAUUAUCCC | | [1561-1579] |
| 27 | ACUGGGAUAAUGAAUUCAA | UUGAAUUCAUUAUCCCAGU | | [1558-1576] |
| 28 | CCAUCCGGAGGUCUUACUU | AAGUAAGACCUCCGGAUGG | | [601-619] |
| 29 | GUGCCUCAUAUUAAUUAUC | GAUAAUUAAUAUGAGGCAC | | [566-584] |
| 30 | CUGUGCCUCAUAUUAAUUA | UAAUUAAUAUGAGGCACAG | | [564-582] |
| 31 | CCGAGUCAAUAAUUCUGAU | AUCAGAAUUAUUGACUCGG | | [401-419] |
| 32 | GGCUUUGUAUGUGAAUAAU | AUUAUUCACAUACAAAGCC | | [4171-4189] |
| 33 | UCUAACCUAUGUGCAUAAU | AUUAUGCACAUAGGUUAGA | | [3788-3806] |
| 34 | UCAGGUAUCAAUUCAUCUA | UAGAUGAAUUGAUACCUGA | | [3645-3663] |
| 35 | CCCAGAUUUCUUAAAGUUU | AAACUUUAAGAAAUCUGGG | | [3316-3334] |
| 36 | GAGCCACAAAUGUUUAGAA | UUCUAAACAUUUGUGGCUC | | [3128-3146] |
| 37 | AGAGCAGGGUUUUCUUUUU | AAAAAGAAAACCCUGCUCU | | [3049-3067] |
| 38 | UCAUCAUCAUCAUCCUAAA | UUUAGGAUGAUGAUGAUGA | | [2920-2938] |
| 39 | CUGUGUGACUAAUGGUUUA | UAAACCAUUAGUCACACAG | | [2890-2908] |
| 40 | CUGGGAUAAUGAAUUCAAG | CUUGAAUUCAUUAUCCCAG | | [1559-1577] |
| 41 | UCGAGCUACAGAUGAAGAA | UUCUUCAUCUGUAGCUCGA | | [937-955] |
| 42 | GGUCUUACUUUGAAGUCUU | AAGACUUCAAAGUAAGACC | | [610-628] |
| 43 | GGGAAUUCUUAAAACUGUG | CACAGUUUUAAGAAUUCCC | | [4221-4239] |
| 44 | GAGUGCUUGUCAUUAUAAA | UUUAUAAUGACAAGCACUC | | [3935-3953] |
| 45 | GGGACUAGGAUGAACUAAA | UUUAGUUCAUCCUAGUCCC | | [3453-3471] |
| 46 | GGAGAAAAGAGAAUGGGAG | CUCCCAUUCUCUUUUCUCC | | [3228-3246] |
| 47 | GGAGCCACAAAUGUUUAGA | UCUAAACAUUUGUGGCUCC | | [3127-3145] |
| 48 | CCCUUAUUUUCCGUAAAUA | UAUUUACGGAAAAUAAGGG | | [2850-2868] |
| 49 | GCCCUUAUGAAUAUUGAAC | GUUCAAUAUUCAUAAGGGC | | [2774-2792] |
| 50 | AACCACACAAUGCCAAGAU | AUCUUGGCAUUGUGUGGUU | | [2472-2490] |
| 51 | UGAAUUUAGAACCACACAA | UUGUGUGGUUCUAAAUUCA | | [2463-2481] |
| 52 | ACAUUUAUGUGGAGCUCUA | UAGAGCUCCACAUAAAUGU | | [1892-1910] |
| 53 | UGAAACCCUGAGUAAACAA | UUGUUUACUCAGGGUUUCA | | [1643-1661] |
| 54 | CUGAAACCCUGAGUAAACA | UGUUUACUCAGGGUUUCAG | | [1642-1660] |
| 55 | GGCUGUGCAUAAUAUAACA | UGUUAUAUUAUGCACAGCC | | [722-740] |
| 56 | GUCUUACUUUGAAGUCUUU | AAAGACUUCAAAGUAAGAC | | [611-629] |
| 57 | UUAAAACUGUGCUCAACUA | UAGUUGAGCACAGUUUUAA | | [4229-4247] |
| 58 | AGGCUUUGUAUGUGAAUAA | UUAUUCACAUACAAAGCCU | | [4170-4188] |
| 59 | GCAUUUAUUAACAGGUACU | AGUACCUGUUAAUAAAUGC | | [4053-4071] |
| 60 | UGUGCAUAAUUAAAACCAG | CUGGUUUUAAUUAUGCACA | | [3797-3815] |
| 61 | UUCUAACCUAUGUGCAUAA | UUAUGCACAUAGGUUAGAA | | [3787-3805] |
| 62 | UCAAUUCUUUACACCAAAG | CUUUGGUGUAAAGAAUUGA | | [3716-3734] |
| 63 | GAAUAUAGCCUUUCUGAAA | UUUCAGAAAGGCUAUAUUC | | [3569-3587] |
| 64 | GGUCUCAUAUUGGUCAUUC | GAAUGACCAAUAUGAGACC | | [3292-3310] |
| 65 | CCAAGAGCAGGGUUUUCUU | AAGAAAACCCUGCUCUUGG | | [3046-3064] |
| 66 | CCACACAAUGCCAAGAUAG | CUAUCUUGGCAUUGUGUGG | | [2474-2492] |
| 67 | UGGAUUGAUUGGAGUUAAU | AUUAACUCCAAUCAAUCCA | | [2373-2391] |
| 68 | CCAGGUUUGAUACUCUUUC | GAAAGAGUAUCAAACCUGG | | [2085-2103] |
| 69 | CUCCAGGUUUGAUACUCUU | AAGAGUAUCAAACCUGGAG | | [2083-2101] |
| 70 | UCAAAAGGUUCAGUAACUU | AAGUUACUGAACCUUUUGA | | [2023-2041] |
| 71 | GGAUCAAAAGGUUCAGUAA | UUACUGAACCUUUUGAUCC | | [2020-2038] |
| 72 | GCAUUUCAUUUUCAACAAG | CUUGUUGAAAAUGAAAUGC | | [1697-1715] |
| 73 | GAAUUCAAGACAAUUGCAA | UUGCAAUUGUCUUGAAUUC | | [1569-1587] |
| 74 | GGAUGAGUCUCAGGCCAAU | AUUGGCCUGAGACUCAUCC | | [1463-1481] |
| 75 | GCCAGAUGCAGGAAAGGAA | UUCCUUUCCUGCAUCUGGC | | [1402-1420] |
| 76 | GAGCUACAGAUGAAGAAGA | UCUUCUUCAUCUGUAGCUC | | [939-957] |
| 77 | GCGGUUUUGGCGAUCUCAA | UUGAGAUCGCCAAAACCGC | | [872-890] |
| 78 | GGAGGUCUUACUUUGAAGU | ACUUCAAAGUAAGACCUCC | Rat, Rb | [607-625] |
| 79 | CGGAGGUCUUACUUUGAAG | CUUCAAAGUAAGACCUCCG | Rat, Rb | [606-624] |
| 80 | AUCACUUCCUCCACCAAAA | UUUUGGUGGAGGAAGUGAU | | [582-600] |
| 81 | GCAGGCUUUGUAUGUGAAU | AUUCACAUACAAAGCCUGC | | [4168-4186] |
| 82 | AGAGUGCUUGUCAUUAUAA | UUAUAAUGACAAGCACUCU | | [3934-3952] |
| 83 | CUAUCUUCAAAACCAACUA | UAGUUGGUUUUGAAGAUAG | | [3854-3872] |
| 84 | GCCCUUUCUAACCUAUGUG | CACAUAGGUUAGAAAGGGC | | [3782-3800] |
| 85 | ACCUCAGGUAUCAAUUCAU | AUGAAUUGAUACCUGAGGU | | [3642-3660] |
| 86 | AGCAGGGUUUUCUUUUUGU | ACAAAAAGAAAACCCUGCU | | [3051-3069] |
| 87 | AUAAAAUGGUGGCAUGGAU | AUCCAUGCCACCAUUUUAU | | [316-334] |
| 88 | UGUGUGACUAAUGGUUUAU | AUAAACCAUUAGUCACACA | | [2891-2909] |
| 89 | AGCUAGCCCUUAUGAAUAU | AUAUUCAUAAGGGCUAGCU | | [2769-2787] |
| 90 | GACUAAUUUCCUGCCCAAA | UUUGGGCAGGAAAUUAGUC | | [2738-2756] |
| 91 | GCAUGAAAAAAGAGGGUUG | CAACCCUCUUUUUUCAUGC | | [2646-2664] |
| 92 | ACAAUGCCAAGAUAGAAUU | AAUUCUAUCUUGGCAUUGU | | [2478-2496] |
| 93 | CACAAUGCCAAGAUAGAAU | AUUCUAUCUUGGCAUUGUG | | [2477-2495] |
| 94 | GAACCACACAAUGCCAAGA | UCUUGGCAUUGUGUGGUUC | | [2471-2489] |
| 95 | ACAUAAUACAUGUUGAGAG | CUCUCAACAUGUAUUAUGU | | [2253-2271] |
| 96 | GGAGUAGCUAUAUAUUUCU | AGAAAUAUAUAGCUACUCC | | [2173-2191] |
| 97 | UGCCUCAGAAUCCUCAAAA | UUUUGAGGAUUCUGAGGCA | | [2117-2135] |
| 98 | GGAGCUCUAUGGUUUUGAG | CUCAAAACCAUAGAGCUCC | | [1902-1920] |
| 99 | GCUGAAACCCUGAGUAAAC | GUUUACUCAGGGUUUCAGC | | [1641-1659] |
| 100 | UCAACACCCUAAUACCAGA | UCUGGUAUUAGGGUGUUGA | Rb | [1589-1607] |
| 101 | CCACCUAAGUUCUUUUACA | UGUAAAAGAACUUAGGUGG | | [120-138] |
| 102 | CGAGCUACAGAUGAAGAAG | CUUCUUCAUCUGUAGCUCG | | [938-956] |
| 103 | GCUGUGCCUCAUAUUAAUU | AAUUAAUAUGAGGCACAGC | | [563-581] |
| 104 | GGCAAAAUGAAAGUUAUCU | AGAUAACUUUCAUUUUGCC | | [454-472] |
| 105 | CAGGCAAAAUGAAAGUUAU | AUAACUUUCAUUUUGCCUG | | [452-470] |
| 106 | ACAGGCAAAAUGAAAGUUA | UAACUUUCAUUUUGCCUGU | | [451-469] |
| 107 | GUCAAUAAUUCUGAUCCUU | AAGGAUCAGAAUUAUUGAC | | [405-423] |
| 108 | CCCGAGUCAAUAAUUCUGA | UCAGAAUUAUUGACUCGGG | | [400-418] |
| 109 | CUGUGCUCAACUAUUAAAA | UUUUAAUAGUUGAGCACAG | | [4235-4253] |
| 110 | GAAUUCUUAAAACUGUGCU | AGCACAGUUUUAAGAAUUC | | [4223-4241] |
| 111 | GGAAUUCUUAAAACUGUGC | GCACAGUUUUAAGAAUUCC | | [4222-4240] |
| 112 | UCUUAGUUCUUCUGCUUUU | AAAAGCAGAAGAACUAAGA | | [4120-4138] |
| 113 | GUGCUUGUCAUUAUAAAAG | CUUUUAUAAUGACAAGCAC | | [3937-3955] |
| 114 | GUUAUAAGGAGUCUGAGAA | UUCUCAGACUCCUUAUAAC | | [3758-3776] |
| 115 | GCUCUGUUUAUUGUUUCAA | UUGAAACAAUAAACAGAGC | | [3701-3719] |
| 116 | CCUCAGGUAUCAAUUCAUC | GAUGAAUUGAUACCUGAGG | | [3643-3661] |
| 117 | GCCUUUCUGAAAUGUACCA | UGGUACAUUUCAGAAAGGC | | [3576-3594] |
| 118 | GGAUGAACUAAAUAAGAAC | GUUCUUAUUUAGUUCAUCC | | [3460-3478] |
| 119 | CACAAAUGUUUAGAACUCU | AGAGUUCUAAACAUUUGUG | | [3132-3150] |
| 120 | GCCACAAAUGUUUAGAACU | AGUUCUAAACAUUUGUGGC | | [3130-3148] |
| 121 | AGCCACAAAUGUUUAGAAC | GUUCUAAACAUUUGUGGCU | | [3129-3147] |
| 122 | GAGCAGGGUUUUCUUUUUG | CAAAAAGAAAACCCUGCUC | | [3050-3068] |
| 123 | CUCAAUAGUAUGUUCUGAA | UUCAGAACAUACUAUUGAG | | [2986-3004] |
| 124 | ACAACCCAGAAACAAAAAU | AUUUUUGUUUCUGGGUUGU | | [2943-2961] |
| 125 | GGAAUUGUGACAAAUAUGU | ACAUAUUUGUCACAAUUCC | | [2796-2814] |
| 126 | GCUAGCCCUUAUGAAUAUU | AAUAUUCAUAAGGGCUAGC | | [2770-2788] |
| 127 | CUCCUUGUGCUAUAAAAUA | UAUUUUAUAGCACAAGGAG | | [2713-2731] |
| 128 | GCUCCUUGUGCUAUAAAAU | AUUUUAUAGCACAAGGAGC | | [2712-2730] |
| 129 | GCCAUCAUUAUUAUCAUAC | GUAUGAUAAUAAUGAUGGC | | [2208-2226] |
| 130 | CCAUCUUACUCCAGGUUUG | CAAACCUGGAGUAAGAUGG | | [2075-2093] |
| 131 | AGGAUCAAAAGGUUCAGUA | UACUGAACCUUUUGAUCCU | | [2019-2037] |
| 132 | GUCGAAAUCUGCUGUCCUU | AAGGACAGCAGAUUUCGAC | | [229-247] |
| 133 | GGGAAAGGAUCAAAAGGUU | AACCUUUUGAUCCUUUCCC | | [2014-2032] |
| 134 | GGAAAAGAUUCUUGGACUC | GAGUCCAAGAAUCUUUUCC | | [1979-1997] |
| 135 | GGGAAAAGAUUCUUGGACU | AGUCCAAGAAUCUUUUCCC | | [1978-1996] |
| 136 | CCUGAGUAAACAAAGCAUC | GAUGCUUUGUUUACUCAGG | | [1649-1667] |
| 137 | UCAGUCUGGUACAAAUAUU | AAUAUUUGUACCAGACUGA | Rb | [1278-1296] |
| 138 | UGGGACAAUACAUUUUUGU | ACAAAAAUGUAUUGUCCCA | | [976-994] |
| 139 | GUGGGACAAUACAUUUUUG | CAAAAAUGUAUUGUCCCAC | | [975-993] |
| 140 | AGUGGGACAAUACAUUUUU | AAAAAUGUAUUGUCCCACU | | [974-992] |
| 141 | CCGGGUUUAUGAUAUUCCA | UGGAAUAUCAUAAACCCGG | | [104-122] |
| 142 | GGAAAAAUAAAGGAAUGCC | GGCAUUCCUUUAUUUUUCC | | [768-786] |
| 143 | GGCUCUCCAUUUUUGUCAU | AUGACAAAAAUGGAGAGCC | | [40-58] |
| 144 | ACGCUGUGCCUCAUAUUAA | UUAAUAUGAGGCACAGCGU | | [561-579] |
| 145 | CGAAAGAGAAUAAAGAACC | GGUUCUUUAUUCUCUUUCG | | [483-501] |
| 146 | GAAUGCCCGAGUCAAUAAU | AUUAUUGACUCGGGCAUUC | | [395-413] |
| 147 | CAGGCUUUGUAUGUGAAUA | UAUUCACAUACAAAGCCUG | | [4169-4187] |
| 148 | GGCUCUGUUUAUUGUUUCA | UGAAACAAUAAACAGAGCC | | [3700-3718] |
| 149 | GUUGUUCUUUGUCAUACUA | UAGUAUGACAAAGAACAAC | | [3482-3500] |
| 150 | UCAGUUGUUCUUUGUCAUA | UAUGACAAAGAACAACUGA | | [3479-3497] |
| 151 | CUCAGUUGUUCUUUGUCAU | AUGACAAAGAACAACUGAG | | [3478-3496] |
| 152 | GAAAAGAGAAUGGGAGAGU | ACUCUCCCAUUCUCUUUUC | | [3231-3249] |
| 153 | GAAAAGGAAAUAAGGAGAA | UUCUCCUUAUUUCCUUUUC | | [3215-3233] |
| 154 | CAAAUGUUUAGAACUCUUC | GAAGAGUUCUAAACAUUUG | | [3134-3152] |
| 155 | GAGUCUCUAAAUCACUGUU | AACAGUGAUUUAGAGACUC | | [3020-3038] |
| 156 | GUAUCUGAUAUGGUCAUUU | AAAUGACCAUAUCAGAUAC | | [2813-2831] |
| 157 | AGCAUGAAAAAAGAGGGUU | AACCCUCUUUUUUCAUGCU | | [2645-2663] |
| 158 | ACACAAUGCCAAGAUAGAA | UUCUAUCUUGGCAUUGUGU | | [2476-2494] |
| 159 | CUACAGAAGAAUAUUGGAU | AUCCAAUAUUCUUCUGUAG | | [2359-2377] |
| 160 | ACCAAGGAGUAGCUAUAUA | UAUAUAGCUACUCCUUGGU | | [2168-2186] |
| 161 | CUGCCUCAGAAUCCUCAAA | UUUGAGGAUUCUGAGGCAG | | [2116-2134] |
| 162 | CCACAAUACUGAGCUGCCU | AGGCAGCUCAGUAUUGUGG | | [2103-2121] |
| 163 | UUGAUACUCUUUCCACAAU | AUUGUGGAAAGAGUAUCAA | | [2091-2109] |
| 164 | GUAAUGGUUUUCCCUUAAA | UUUAAGGGAAAACCAUUAC | | [1840-1858] |
| 165 | CCAUGAGGAAAUAAAUGUG | CACAUUUAUUUCCUCAUGG | | [1738-1756] |
| 166 | GGAGUGCAUUUCAUUUUCA | UGAAAAUGAAAUGCACUCC | | [1692-1710] |
| 167 | GUAAACAAAGCAUCUCCAA | UUGGAGAUGCUUUGUUUAC | | [1654-1672] |
| 168 | GGAGAAAGAUGUGAUCACA | UGUGAUCACAUCUUUCUCC | Rb, Ms | [1505-1523] |
| 169 | CCAUUCUCAAGUCAGUCUG | CAGACUGACUUGAGAAUGG | | [1267-1285] |
| 170 | UCAAGAUGCGUGGAAACUA | UAGUUUCCACGCAUCUUGA | | [1142-1160] |
| 171 | AGUUUCAAGAUGCGUGGAA | UUCCACGCAUCUUGAAACU | | [1138-1156] |
| 172 | GGAGUUUCAAGAUGCGUGG | CCACGCAUCUUGAAACUCC | | [1136-1154] |
| 173 | AGACUUCUUUAGUAUCCAU | AUGGAUACUAAAGAAGUCU | | [1058-1076] |
| 174 | GAGGAAGACUUCUUUAGUA | UACUAAAGAAGUCUUCCUC | | [1053-1071] |
| 175 | AAACCAUCGAGCUACAGAU | AUCUGUAGCUCGAUGGUUU | | [931-949] |
| 176 | AGAGGUUGGUGCGGUUUUG | CAAAACCGCACCAACCUCU | | [862-880] |
| 177 | GAGAGGUUGGUGCGGUUUU | AAAACCGCACCAACCUCUC | | [861-879] |
| 178 | GCUCUCCAUUUUUGUCAUU | AAUGACAAAAAUGGAGAGC | | [41-59] |
| 179 | GUGAACAAAAAAUCUCAGA | UCUGAGAUUUUUUGUUCAC | | [745-763] |
| 180 | CACGCUGUGCCUCAUAUUA | UAAUAUGAGGCACAGCGUG | | [560-578] |
| 181 | GCCCGAGUCAAUAAUUCUG | CAGAAUUAUUGACUCGGGC | | [399-417] |
| 182 | GUGCUCAACUAUUAAAAUG | CAUUUUAAUAGUUGAGCAC | | [4237-4255] |
| 183 | ACUGUGCUCAACUAUUAAA | UUUAAUAGUUGAGCACAGU | | [4234-4252] |
| 184 | UGCAGGCUUUGUAUGUGAA | UUCACAUACAAAGCCUGCA | | [4167-4185] |
| 185 | GGUUUUUCUUAGUUCUUCU | AGAAGAACUAAGAAAAACC | | [4114-4132] |
| 186 | AGGUUUUUCUUAGUUCUUC | GAAGAACUAAGAAAAACCU | | [4113-4131] |
| 187 | GAAGGUUUUUCUUAGUUCU | AGAACUAAGAAAAACCUUC | | [4111-4129] |
| 188 | GAAUGAUGAAGCAUUGUUU | AAACAAUGCUUCAUCAUUC | | [4076-4094] |
| 189 | AGAAUGAUGAAGCAUUGUU | AACAAUGCUUCAUCAUUCU | | [4075-4093] |
| 190 | CUAAGAAUGAUGAAGCAUU | AAUGCUUCAUCAUUCUUAG | | [4072-4090] |
| 191 | AUAUAAGAGUGCUUGUCAU | AUGACAAGCACUCUUAUAU | | [3929-3947] |
| 192 | GAAUAUAAGAGUGCUUGUC | GACAAGCACUCUUAUAUUC | | [3927-3945] |
| 193 | CCCUUUCUAACCUAUGUGC | GCACAUAGGUUAGAAAGGG | | [3783-3801] |
| 194 | AUAUAGCCUUUCUGAAAUG | CAUUUCAGAAAGGCUAUAU | | [3571-3589] |
| 195 | AGGCCUGCUGUGCGAAUAU | AUAUUCGCACAGCAGGCCU | | [3556-3574] |
| 196 | CUAGGAUGAACUAAAUAAG | CUUAUUUAGUUCAUCCUAG | | [3457-3475] |
| 197 | GGAUUAUUCAAGGGACUAG | CUAGUCCCUUGAAUAAUCC | | [3442-3460] |
| 198 | GGAGAGUGAGAGAAAAUAA | UUAUUUUCUCUCACUCUCC | | [3243-3261] |
| 199 | GGGAGAGUGAGAGAAAAUA | UAUUUUCUCUCACUCUCCC | | [3242-3260] |
| 200 | AGAAAAGAGAAUGGGAGAG | CUCUCCCAUUCUCUUUUCU | | [3230-3248] |
| 201 | GGAAAUAAGGAGAAAAGAG | CUCUUUUCUCCUUAUUUCC | | [3220-3238] |
| 202 | GGAAAAGGAAAUAAGGAGA | UCUCCUUAUUUCCUUUUCC | | [3214-3232] |
| 203 | GAACUCUUCAACUUCGGUA | UACCGAAGUUGAAGAGUUC | | [3144-3162] |
| 204 | UGGAGCCACAAAUGUUUAG | CUAAACAUUUGUGGCUCCA | | [3126-3144] |
| 205 | GGUUGAACGUCUUCCUCUU | AAGAGGAAGACGUUCAACC | | [73-91] |
| 206 | CAAGAGCAGGGUUUUCUUU | AAAGAAAACCCUGCUCUUG | | [3047-3065] |
| 207 | AGUCUCUAAAUCACUGUUA | UAACAGUGAUUUAGAGACU | | [3021-3039] |
| 208 | AGAGUCUCUAAAUCACUGU | ACAGUGAUUUAGAGACUCU | | [3019-3037] |
| 209 | UAACAACCCAGAAACAAAA | UUUUGUUUCUGGGUUGUUA | | [2941-2959] |
| 210 | CUAAAAUUAACAACCCAGA | UCUGGGUUGUUAAUUUUAG | | [2934-2952] |
| 211 | UCCUAAAAUUAACAACCCA | UGGGUUGUUAAUUUUAGGA | | [2932-2950] |
| 212 | AGCCCUUAUGAAUAUUGAA | UUCAAUAUUCAUAAGGGCU | | [2773-2791] |
| 213 | CCAGCUAGCCCUUAUGAAU | AUUCAUAAGGGCUAGCUGG | | [2767-2785] |
| 214 | CAAUGCCAAGAUAGAAUUA | UAAUUCUAUCUUGGCAUUG | | [2479-2497] |
| 215 | GGAUUGAUUGGAGUUAAUG | CAUUAACUCCAAUCAAUCC | | [2374-2392] |
| 216 | GAAUCCUCAAAAUCAGUUU | AAACUGAUUUUGAGGAUUC | | [2124-2142] |
| 217 | CAGUGGAAGUUAGGGAAAA | UUUUCCCUAACUUCCACUG | | [1966-1984] |
| 218 | AGAGCACUUUUACAAACAU | AUGUUUGUAAAAGUGCUCU | | [1919-1937] |
| 219 | GGUUUUGAGAGCACUUUUA | UAAAAGUGCUCUCAAAACC | | [1912-1930] |
| 220 | ACUGCAUGCUGAUUCUCUU | AAGAGAAUCAGCAUGCAGU | | [202-220] |
| 221 | UGUGAUCACAGGCCUGAAA | UUUCAGGCCUGUGAUCACA | | [1514-1532] |
| 222 | CCAUGAUGAGGAGAAAGAU | AUCUUUCUCCUCAUCAUGG | Rb, Ms | [1496-1514] |
| 223 | UGAAUUUCAACUGCAUGCU | AGCAUGCAGUUGAAAUUCA | Rat | [193-211] |
| 224 | CGCAUCCAUUCUCAAGUCA | UGACUUGAGAAUGGAUGCG | | [1262-1280] |
| 225 | CCCUUCGCAUCCAUUCUCA | UGAGAAUGGAUGCGAAGGG | | [1257-1275] |
| 226 | GUUUCAAGAUGCGUGGAAA | UUUCCACGCAUCUUGAAAC | | [1139-1157] |
| 227 | GCAGGAGUUUCAAGAUGCG | CGCAUCUUGAAACUCCUGC | | [1133-1151] |
| 228 | AGGAAGACUUCUUUAGUAU | AUACUAAAGAAGUCUUCCU | | [1054-1072] |
| 229 | CCAUGUUUCUGUAUCUCUG | CAGAGAUACAGAAACAUGG | | [841-859] |
| 230 | UGGCUGUGCAUAAUAUAAC | GUUAUAUUAUGCACAGCCA | | [721-739] |
| 231 | GGUACACACAUCAUCUCUU | AAGAGAUGAUGUGUGUACC | | [631-649] |
| 232 | AGGUCUUACUUUGAAGUCU | AGACUUCAAAGUAAGACCU | | [609-627] |
| 233 | GGAGUUGUCAUCACGCUGU | ACAGCGUGAUGACAACUCC | | [549-567] |
| 234 | UCGAAAGAGAAUAAAGAAC | GUUCUUUAUUCUCUUUCGA | | [482-500] |
| 235 | GGAGACAGGCAAAAUGAAA | UUUCAUUUUGCCUGUCUCC | | [447-465] |
| 236 | AGUCAAUAAUUCUGAUCCU | AGGAUCAGAAUUAUUGACU | | [404-422] |
| 237 | UGAAUGCCCGAGUCAAUAA | UUAUUGACUCGGGCAUUCA | | [394-412] |
| 238 | GCUUUUGCAAUUGUGUUUG | CAAACACAAUUGCAAAAGC | | [4133-4151] |
| 239 | UGAAUAUAAGAGUGCUUGU | ACAAGCACUCUUAUAUUCA | | [3926-3944] |
| 240 | CUCAGUUAUAAGGAGUCUG | CAGACUCCUUAUAACUGAG | | [3754-3772] |
| 241 | UCACACCAUUGCACAUCUA | UAGAUGUGCAAUGGUGUGA | | [356-374] |
| 242 | GCCUGCUGUGCGAAUAUAG | CUAUAUUCGCACAGCAGGC | | [3558-3576] |
| 243 | AGGGUAGGUCUGACAAAUA | UAUUUGUCAGACCUACCCU | | [3537-3555] |
| 244 | CAGUUGUUCUUUGUCAUAC | GUAUGACAAAGAACAACUG | | [3480-3498] |
| 245 | GACUAGGAUGAACUAAAUA | UAUUUAGUUCAUCCUAGUC | | [3455-3473] |
| 246 | GGGAUUAUUCAAGGGACUA | UAGUCCCUUGAAUAAUCCC | | [3441-3459] |
| 247 | ACAUACUGAUGUUGUUUUG | CAAAACAACAUCAGUAUGU | | [3368-3386] |
| 248 | GAGAGUGAGAGAAAAUAAA | UUUAUUUUCUCUCACUCUC | | [3244-3262] |
| 249 | UGAGGAAGAAGGAGAAAGA | UCUUUCUCCUUCUUCCUCA | | [3164-3182] |
| 250 | GAAUAUAUGUUCAAGAGAG | CUCUCUUGAACAUAUAUUC | | [3002-3020] |
| 251 | CAUCAUCAUCAUCCUAAAA | UUUUAGGAUGAUGAUGAUG | | [2921-2939] |
| 252 | ACUUAGGAAUUGUGACAAA | UUUGUCACAAUUCCUAAGU | | [2791-2809] |
| 253 | CUAGCCCUUAUGAAUAUUG | CAAUAUUCAUAAGGGCUAG | | [2771-2789] |
| 254 | UGAAAAAAGAGGGUUGGAG | CUCCAACCCUCUUUUUUCA | | [2649-2667] |
| 255 | GGGUUCUGCACAUUCUUAU | AUAAGAAUGUGCAGAACCC | | [2622-2640] |
| 256 | UUGGAUUGAUUGGAGUUAA | UUAACUCCAAUCAAUCCAA | | [2372-2390] |
| 257 | GUGUCAUUUUUGCCAUCAU | AUGAUGGCAAAAAUGACAC | | [2197-2215] |
| 258 | AGAAUCCUCAAAAUCAGUU | AACUGAUUUUGAGGAUUCU | | [2123-2141] |
| 259 | AGGUUUGAUACUCUUUCCA | UGGAAAGAGUAUCAAACCU | | [2087-2105] |
| 260 | UCCAGGUUUGAUACUCUUU | AAAGAGUAUCAAACCUGGA | | [2084-2102] |
| 261 | GUAACUUCCCUAAGAUUAU | AUAAUCUUAGGGAAGUUAC | | [2035-2053] |
| 262 | CAAAAGGGAAAGGAUCAAA | UUUGAUCCUUUCCCUUUUG | | [2009-2027] |
| 263 | AAAGAUUCUUGGACUCAAU | AUUGAGUCCAAGAAUCUUU | | [1982-2000] |
| 264 | GGAAGUUAGGGAAAAGAUU | AAUCUUUUCCCUAACUUCC | | [1970-1988] |
| 265 | GAGCACUUUUACAAACAUU | AAUGUUUGUAAAAGUGCUC | | [1920-1938] |
| 266 | GGAAUGUCAAAGAUUGUUU | AAACAAUCUUUGACAUUCC | | [1859-1877] |
| 267 | GAGUGCAUUUCAUUUUCAA | UUGAAAAUGAAAUGCACUC | | [1693-1711] |
| 268 | GGGAGUGCAUUUCAUUUUC | GAAAAUGAAAUGCACUCCC | | [1691-1709] |
| 269 | GACUUCUUUAGUAUCCAUA | UAUGGAUACUAAAGAAGUC | | [1059-1077] |
| 270 | GCACCCUUUUACACUGACA | UGUCAGUGUAAAAGGGUGC | | [1025-1043] |
| 271 | CCUAAGUUCUUUUACACAA | UUGUGUAAAAGAACUUAGG | | [123-141] |
| 272 | AAGUGGGACAAUACAUUUU | AAAAUGUAUUGUCCCACUU | | [973-991] |
| 273 | CAAAAUGGAAGUGGGACAA | UUGUCCCACUUCCAUUUUG | | [965-983] |
| 274 | CUCACCCUUUCAAAACCAU | AUGGUUUUGAAAGGGUGAG | Rb | [919-937] |
| 275 | CGGUUUUGGCGAUCUCAAC | GUUGAGAUCGCCAAAACCG | | [873-891] |
| 276 | UGAGAGGUUGGUGCGGUUU | AAACCGCACCAACCUCUCA | | [860-878] |
| 277 | AGAGAGUUUGGCUGUGCAU | AUGCACAGCCAAACUCUCU | | [713-731] |
| 278 | CAGAGAGUUUGGCUGUGCA | UGCACAGCCAAACUCUCUG | | [712-730] |
| 279 | ACACACAUCAUCUCUUUGU | ACAAAGAGAUGAUGUGUGU | | [634-652] |
| 280 | ACUUUGAAGUCUUUUGGUA | UACCAAAAGACUUCAAAGU | | [616-634] |
| 281 | UCACUGGAGUUGUCAUCAC | GUGAUGACAACUCCAGUGA | | [544-562] |
| 282 | UCCUCUUUGUCUGGUAUUA | UAAUACCAGACAAAGAGGA | | [85-103] |
| 283 | GACAGGCAAAAUGAAAGUU | AACUUUCAUUUUGCCUGUC | | [450-468] |
| 284 | UGCAAUUGUGUUUGUGAAA | UUUCACAAACACAAUUGCA | | [4138-4156] |
| 285 | UUGCAAUUGUGUUUGUGAA | UUCACAAACACAAUUGCAA | | [4137-4155] |
| 286 | CUUUUGCAAUUGUGUUUGU | ACAAACACAAUUGCAAAAG | | [4134-4152] |
| 287 | CUUAAGACUAUGAAGGUUU | AAACCUUCAUAGUCUUAAG | | [4100-4118] |
| 288 | AGCAUUUAUUAACAGGUAC | GUACCUGUUAAUAAAUGCU | | [4052-4070] |
| 289 | AGCACUUAGUAUUUAGCAU | AUGCUAAAUACUAAGUGCU | | [4038-4056] |
| 290 | CCAAGGUUUUCUAUCUUCA | UGAAGAUAGAAAACCUUGG | | [3844-3862] |
| 291 | CUCCAAGGUUUUCUAUCUU | AAGAUAGAAAACCUUGGAG | | [3842-3860] |
| 292 | AGAAUAUUGGCCCUUUCUA | UAGAAAGGGCCAAUAUUCU | | [3773-3791] |
| 293 | ACCAUUGCACAUCUAUUUA | UAAAUAGAUGUGCAAUGGU | | [360-378] |
| 294 | AGUUGUUCUUUGUCAUACU | AGUAUGACAAAGAACAACU | | [3481-3499] |
| 295 | GAAUAUAAUUGAAGGAGGU | ACCUCCUUCAAUUAUAUUC | | [3345-3363] |
| 296 | GAAUGGGAGAGUGAGAGAA | UUCUCUCACUCUCCCAUUC | | [3238-3256] |
| 297 | CUUGGAGCCACAAAUGUUU | AAACAUUUGUGGCUCCAAG | | [3124-3142] |
| 298 | GUCUCUAAAUCACUGUUAG | CUAACAGUGAUUUAGAGAC | | [3022-3040] |
| 299 | GAGAGUCUCUAAAUCACUG | CAGUGAUUUAGAGACUCUC | | [3018-3036] |
| 300 | CAACCCAGAAACAAAAAUC | GAUUUUUGUUUCUGGGUUG | | [2944-2962] |
| 301 | UGUAUCUGAUAUGGUCAUU | AAUGACCAUAUCAGAUACA | | [2812-2830] |
| 302 | CUCAUCUAGUCCUGCUCCU | AGGAGCAGGACUAGAUGAG | | [2699-2717] |
| 303 | UGCACAUUCUUAUUUGAAG | CUUCAAAUAAGAAUGUGCA | | [2628-2646] |
| 304 | CUGCACAUUCUUAUUUGAA | UUCAAAUAAGAAUGUGCAG | | [2627-2645] |
| 305 | CCUGAAGGGUUCUGCACAU | AUGUGCAGAACCCUUCAGG | | [2616-2634] |
| 306 | UACUAAUACAGCAGGGUAA | UUACCCUGCUGUAUUAGUA | | [2322-2340] |
| 307 | CAAGGAGUAGCUAUAUAUU | AAUAUAUAGCUACUCCUUG | | [2170-2188] |
| 308 | UCAGAAUCCUCAAAAUCAG | CUGAUUUUGAGGAUUCUGA | | [2121-2139] |
| 309 | CCCUAAGAUUAUGAAACUG | CAGUUUCAUAAUCUUAGGG | | [2042-2060] |
| 310 | CAGUAACUUCCCUAAGAUU | AAUCUUAGGGAAGUUACUG | | [2033-2051] |
| 311 | AUCAAAAGGUUCAGUAACU | AGUUACUGAACCUUUUGAU | | [2022-2040] |
| 312 | UCAAAAGGGAAAGGAUCAA | UUGAUCCUUUCCCUUUUGA | | [2008-2026] |
| 313 | AAAAGAUUCUUGGACUCAA | UUGAGUCCAAGAAUCUUUU | | [1981-1999] |
| 314 | CUGCUUAAAAAUGGACAAA | UUUGUCCAUUUUUAAGCAG | | [1808-1826] |
| 315 | CCUGCUUAAAAAUGGACAA | UUGUCCAUUUUUAAGCAGG | | [1807-1825] |
| 316 | GAGGAAAUAAAUGUGGGUU | AACCCACAUUUAUUUCCUC | | [1742-1760] |
| 317 | CAACAAGGAAAACUUCUAA | UUAGAAGUUUUCCUUGUUG | Rat, Ms | [1709-1727] |
| 318 | GUGCAUUUCAUUUUCAACA | UGUUGAAAAUGAAAUGCAC | | [1695-1713] |
| 319 | ACAGGCCUGAAACAAAAGA | UCUUUUGUUUCAGGCCUGU | | [1521-1539] |
| 320 | UCCUCAGCUACAACAUCUA | UAGAUGUUGUAGCUGAGGA | Rat, Rb | [1432-1450] |
| 321 | AGAUGCAGGAAAGGAACAA | UUGUUCCUUUCCUGCAUCU | Ms | [1405-1423] |
| 322 | CCAGAUGCAGGAAAGGAAC | GUUCCUUUCCUGCAUCUGG | Ms | [1403-1421] |
| 323 | UCGCAUCCAUUCUCAAGUC | GACUUGAGAAUGGAUGCGA | | [1261-1279] |
| 324 | UCAGCUAUGAGGUGGUGAU | AUCACCACCUCAUAGCUGA | Rat, Rb, Ms | [1210-1228] |
| 325 | CUCCAUUUUUGUCAUUCUG | CAGAAUGACAAAAAUGGAG | | [44-62] |
| 326 | CACCCUUUUACACUGACAU | AUGUCAGUGUAAAAGGGUG | | [1026-1044] |
| 327 | UUCCACCUAAGUUCUUUUA | UAAAAGAACUUAGGUGGAA | | [118-136] |
| 328 | GUCCCAUGUUUCUGUAUCU | AGAUACAGAAACAUGGGAC | | [838-856] |
| 329 | GGGUCCCAUGUUUCUGUAU | AUACAGAAACAUGGGACCC | | [836-854] |
| 330 | GUUUAUGAUAUUCCACCUA | UAGGUGGAAUAUCAUAAAC | | [108-126] |
| 331 | CGGGUUUAUGAUAUUCCAC | GUGGAAUAUCAUAAACCCG | | [105-123] |
| 332 | AGAGUUUGGCUGUGCAUAA | UUAUGCACAGCCAAACUCU | | [715-733] |
| 333 | UGAAGUCUUUUGGUACACA | UGUGUACCAAAAGACUUCA | | [620-638] |
| 334 | GCAUCACUGGAGUUGUCAU | AUGACAACUCCAGUGAUGC | | [541-559] |
| 335 | AGACAGGCAAAAUGAAAGU | ACUUUCAUUUUGCCUGUCU | | [449-467] |
| 336 | UGAACUUGGAGACAGGCAA | UUGCCUGUCUCCAAGUUCA | | [440-458] |
| 337 | UGAUCCUUAUUCAGUAGCA | UGCUACUGAAUAAGGAUCA | | [416-434] |
| 338 | GGACCUGGGAGAUAAUUCU | AGAAUUAUCUCCCAGGUCC | | [4199-4217] |
| 339 | UCUUAAGACUAUGAAGGUU | AACCUUCAUAGUCUUAAGA | | [4099-4117] |
| 340 | CUCUAAGAAUGAUGAAGCA | UGCUUCAUCAUUCUUAGAG | | [4070-4088] |
| 341 | GUACUCUAAGAAUGAUGAA | UUCAUCAUUCUUAGAGUAC | | [4067-4085] |
| 342 | CAGGUACUCUAAGAAUGAU | AUCAUUCUUAGAGUACCUG | | [4064-4082] |
| 343 | AAAACCAGCUUCAUUUGUU | AACAAAUGAAGCUGGUUUU | | [3808-3826] |
| 344 | GUGUUAUUACUAUGAGUCA | UGACUCAUAGUAAUAACAC | | [3681-3699] |
| 345 | UGCUUAGAGACACUUAGGU | ACCUAAGUGUCUCUAAGCA | | [3605-3623] |
| 346 | GGAUGGUUUCUGCUUAGAG | CUCUAAGCAGAAACCAUCC | | [3595-3613] |
| 347 | UCAUACUACUAUUCCUUUC | GAAAGGAAUAGUAGUAUGA | | [3493-3511] |
| 348 | UGUCAUACUACUAUUCCUU | AAGGAAUAGUAGUAUGACA | | [3491-3509] |
| 349 | GAAGGAGGUAUACACAUAC | GUAUGUGUAUACCUCCUUC | | [3355-3373] |
| 350 | AAGGAAAUAAGGAGAAAAG | CUUUUCUCCUUAUUUCCUU | | [3218-3236] |
| 351 | GACUGGUUUAGGAGGAAAA | UUUUCCUCCUAAACCAGUC | | [3201-3219] |
| 352 | CGGUAAUGAGGAAGAAGGA | UCCUUCUUCCUCAUUACCG | | [3158-3176] |
| 353 | CUCUUCAACUUCGGUAAUG | CAUUACCGAAGUUGAAGAG | | [3147-3165] |
| 354 | CAUGGAUGAUUGCACUUCA | UGAAGUGCAAUCAUCCAUG | | [328-346] |
| 355 | CCUUGGAGCCACAAAUGUU | AACAUUUGUGGCUCCAAGG | | [3123-3141] |
| 356 | CCCUUGGAGCCACAAAUGU | ACAUUUGUGGCUCCAAGGG | | [3122-3140] |
| 357 | CAAAUUCACACUCAAUAGU | ACUAUUGAGUGUGAAUUUG | | [2976-2994] |
| 358 | AUGUAUCUGAUAUGGUCAU | AUGACCAUAUCAGAUACAU | | [2811-2829] |
| 359 | CUUAGGAAUUGUGACAAAU | AUUUGUCACAAUUCCUAAG | | [2792-2810] |
| 360 | UCAUCUAGUCCUGCUCCUU | AAGGAGCAGGACUAGAUGA | | [2700-2718] |
| 361 | GGGUUGGAGGUGGAGAAUU | AAUUCUCCACCUCCAACCC | | [2659-2677] |
| 362 | CAUUCUUAUUUGAAGCAUG | CAUGCUUCAAAUAAGAAUG | | [2632-2650] |
| 363 | GUAAUACUCAUCAUUUACC | GGUAAAUGAUGAGUAUUAC | | [2392-2410] |
| 364 | AAAAGAAGAAGGAAACCAA | UUGGUUUCCUUCUUCUUUU | | [2154-2172] |
| 365 | CAAAAGAAGAAGGAAACCA | UGGUUUCCUUCUUCUUUUG | | [2153-2171] |
| 366 | GCUGCCUCAGAAUCCUCAA | UUGAGGAUUCUGAGGCAGC | | [2115-2133] |
| 367 | CAGGUUUGAUACUCUUUCC | GGAAAGAGUAUCAAACCUG | | [2086-2104] |
| 368 | UCUUACUCCAGGUUUGAUA | UAUCAAACCUGGAGUAAGA | | [2078-2096] |
| 369 | CCUAAGAUUAUGAAACUGU | ACAGUUUCAUAAUCUUAGG | | [2043-2061] |
| 370 | UCCCUAAGAUUAUGAAACU | AGUUUCAUAAUCUUAGGGA | | [2041-2059] |
| 371 | AAAAGGGAAAGGAUCAAAA | UUUUGAUCCUUUCCCUUUU | | [2010-2028] |
| 372 | AGGGAAAAGAUUCUUGGAC | GUCCAAGAAUCUUUUCCCU | | [1977-1995] |
| 373 | UCAGUGGAAGUUAGGGAAA | UUUCCCUAACUUCCACUGA | | [1965-1983] |
| 374 | CUAUGGUUUUGAGAGCACU | AGUGCUCUCAAAACCAUAG | | [1908-1926] |
| 375 | UGGAGCUCUAUGGUUUUGA | UCAAAACCAUAGAGCUCCA | | [1901-1919] |
| 376 | GUGGAGCUCUAUGGUUUUG | CAAAACCAUAGAGCUCCAC | | [1900-1918] |
| 377 | CUCUUGCCAGUCUGUCGAA | UUCGACAGACUGGCAAGAG | | [216-234] |
| 378 | AGGAAAUAAAUGUGGGUUG | CAACCCACAUUUAUUUCCU | | [1743-1761] |
| 379 | UGAUUCUCUUGCCAGUCUG | CAGACUGGCAAGAGAAUCA | | [211-229] |
| 380 | AAAGGAACAAUGCCGGCUU | AAGCCGGCAUUGUUCCUUU | | [1414-1432] |
| 381 | GUCAGUCUGGUACAAAUAU | AUAUUUGUACCAGACUGAC | Rb | [1277-1295] |
| 382 | CAAGUCAGUCUGGUACAAA | UUUGUACCAGACUGACUUG | Rb | [1274-1292] |
| 383 | GGAAGACUUCUUUAGUAUC | GAUACUAAAGAAGUCUUCC | | [1055-1073] |
| 384 | CCUGAGGAAGACUUCUUUA | UAAAGAAGUCUUCCUCAGG | Rat | [1050-1068] |
| 385 | GGCACCCUUUUACACUGAC | GUCAGUGUAAAAGGGUGCC | | [1024-1042] |
| 386 | CACCUAAGUUCUUUUACAC | GUGUAAAAGAACUUAGGUG | | [121-139] |
| 387 | AGCUACAGAUGAAGAAGAA | UUCUUCUUCAUCUGUAGCU | | [940-958] |
| 388 | CUCAACAGAAGGUGGUCAU | AUGACCACCUUCUGUUGAG | | [886-904] |
| 389 | GGCGAUCUCAACAGAAGGU | ACCUUCUGUUGAGAUCGCC | | [880-898] |
| 390 | UGAUAUUCCACCUAAGUUC | GAACUUAGGUGGAAUAUCA | | [113-131] |
| 391 | CCCAUGUUUCUGUAUCUCU | AGAGAUACAGAAACAUGGG | | [840-858] |
| 392 | GGUCCCAUGUUUCUGUAUC | GAUACAGAAACAUGGGACC | | [837-855] |
| 393 | GUUUGGCUGUGCAUAAUAU | AUAUUAUGCACAGCCAAAC | | [718-736] |
| 394 | UCUUUGUGAUCUUCUUCAU | AUGAAGAAGAUCACAAAGA | Rat, Ms | [646-664] |
| 395 | AUCUCUUUGUGAUCUUCUU | AAGAAGAUCACAAAGAGAU | Rat, Ms | [643-661] |
| 396 | UCAUCUCUUUGUGAUCUUC | GAAGAUCACAAAGAGAUGA | | [641-659] |
| 397 | GAAGUCUUUUGGUACACAC | GUGUGUACCAAAAGACUUC | | [621-639] |
| 398 | AGGCAAAAUGAAAGUUAUC | GAUAACUUUCAUUUUGCCU | | [453-471] |
| 399 | GGGCUCUCCAUUUUUGUCA | UGACAAAAAUGGAGAGCCC | | [39-57] |
| 400 | GGUACUCUAAGAAUGAUGA | UCAUCAUUCUUAGAGUACC | | [4066-4084] |
| 401 | CAAGGUUUUCUAUCUUCAA | UUGAAGAUAGAAAACCUUG | | [3845-3863] |
| 402 | CUAUUUAAUGUGGAAUGGU | ACCAUUCCACAUUAAAUAG | | [372-390] |
| 403 | AGGCUCUGUUUAUUGUUUC | GAAACAAUAAACAGAGCCU | | [3699-3717] |
| 404 | ACACCAUUGCACAUCUAUU | AAUAGAUGUGCAAUGGUGU | | [358-376] |
| 405 | AGGAUGGUUUCUGCUUAGA | UCUAAGCAGAAACCAUCCU | | [3594-3612] |
| 406 | UGAGGGUAGGUCUGACAAA | UUUGUCAGACCUACCCUCA | | [3535-3553] |
| 407 | GUCAUACUACUAUUCCUUU | AAAGGAAUAGUAGUAUGAC | | [3492-3510] |
| 408 | ACUCAGUUGUUCUUUGUCA | UGACAAAGAACAACUGAGU | | [3477-3495] |
| 409 | CUGGUCACAAAUUUUGAUG | CAUCAAAAUUUGUGACCAG | | [3416-3434] |
| 410 | UCUGGUCACAAAUUUUGAU | AUCAAAAUUUGUGACCAGA | | [3415-3433] |
| 411 | AGACUGGUUUAGGAGGAAA | UUUCCUCCUAAACCAGUCU | | [3200-3218] |
| 412 | UAAUGAGGAAGAAGGAGAA | UUCUCCUUCUUCCUCAUUA | | [3161-3179] |
| 413 | GUAAUGAGGAAGAAGGAGA | UCUCCUUCUUCCUCAUUAC | | [3160-3178] |
| 414 | CUUCGGUAAUGAGGAAGAA | UUCUUCCUCAUUACCGAAG | | [3155-3173] |
| 415 | AACUCUUCAACUUCGGUAA | UUACCGAAGUUGAAGAGUU | | [3145-3163] |
| 416 | ACAGAGAUCAAAUUCACAC | GUGUGAAUUUGAUCUCUGU | | [2968-2986] |
| 417 | CUGAAGGGUUCUGCACAUU | AAUGUGCAGAACCCUUCAG | | [2617-2635] |
| 418 | AGGGUCAAGAACAGGCUAA | UUAGCCUGUUCUUGACCCU | | [2576-2594] |
| 419 | UCAUUUACCACUGUGCUUG | CAAGCACAGUGGUAAAUGA | | [2402-2420] |
| 420 | CUCAUCAUUUACCACUGUG | CACAGUGGUAAAUGAUGAG | | [2398-2416] |
| 421 | CAGAAGAAUAUUGGAUUGA | UCAAUCCAAUAUUCUUCUG | | [2362-2380] |
| 422 | CCUCAGAAUCCUCAAAAUC | GAUUUUGAGGAUUCUGAGG | | [2119-2137] |
| 423 | CUAAGAUUAUGAAACUGUG | CACAGUUUCAUAAUCUUAG | | [2044-2062] |
| 424 | AGAUUCUUGGACUCAAUUU | AAAUUGAGUCCAAGAAUCU | | [1984-2002] |
| 425 | GUCAGUGGAAGUUAGGGAA | UUCCCUAACUUCCACUGAC | | [1964-1982] |
| 426 | CCCUGCUUAAAAAUGGACA | UGUCCAUUUUUAAGCAGGG | | [1806-1824] |
| 427 | UGAGGAAAUAAAUGUGGGU | ACCCACAUUUAUUUCCUCA | | [1741-1759] |
| 428 | UCUAACUUGUCUCUUCCAU | AUGGAAGAGACAAGUUAGA | | [1723-1741]+ |
| 429 | CAAGGAAAACUUCUAACUU | AAGUUAGAAGUUUUCCUUG | | [1712-1730]+ |
| 430 | ACAAGGAAAACUUCUAACU | AGUUAGAAGUUUUCCUUGU | Ms | [1711-1729]+ |
| 431 | UCAACAAGGAAAACUUCUA | UAGAAGUUUUCCUUGUUGA | Rat, Ms | [1708-1726] |
| 432 | UGAGGAGAAAGAUGUGAUC | GAUCACAUCUUUCUCCUCA | Rb, Ms | [1502-1520] |
| 433 | UCAGCUACAACAUCUACCU | AGGUAGAUGUUGUAGCUGA | Rat | [1435-1453] |
| 434 | GCCUUUGAGUGGUUUGCAG | CUGCAAACCACUCAAAGGC | Rb, Ms | [1362-1380] |
| 435 | ACAUGCCUUUGAGUGGUUU | AAACCACUCAAAGGCAUGU | Rb | [1358-1376] |
| 436 | AAGUCAGUCUGGUACAAAU | AUUUGUACCAGACUGACUU | Rb | [1275-1293] |
| 437 | UCCAUUCUCAAGUCAGUCU | AGACUGACUUGAGAAUGGA | | [1266-1284] |
| 438 | ACCCUUCGCAUCCAUUCUC | GAGAAUGGAUGCGAAGGGU | | [1256-1274] |
| 439 | CUGCCAGUGAAGAUGUGUU | AACACAUCUUCACUGGCAG | | [1192-1210] |
| 440 | UGGAAGUGGGACAAUACAU | AUGUAUUGUCCCACUUCCA | | [970-988] |
| 441 | UCAAAAUGGAAGUGGGACA | UGUCCCACUUCCAUUUUGA | | [964-982] |
| 442 | GGGUUCAAAAUGGAAGUGG | CCACUUCCAUUUUGAACCC | | [960-978] |
| 443 | GUCACUCACCCUUUCAAAA | UUUUGAAAGGGUGAGUGAC | Rb | [915-933] |
| 444 | UCCCAUGUUUCUGUAUCUC | GAGAUACAGAAACAUGGGA | | [839-857] |
| 445 | UGGGUCCCAUGUUUCUGUA | UACAGAAACAUGGGACCCA | | [835-853] |
| 446 | CCUAUGACUUGGAAAUGGA | UCCAUUUCCAAGUCAUAGG | Rat, Ms | [813-831] |
| 447 | CUCUUUGUGAUCUUCUUCA | UGAAGAAGAUCACAAAGAG | Rat, Ms | [645-663] |
| 448 | GUCUUUUGGUACACACAUC | GAUGUGUGUACCAAAAGAC | | [624-642] |
| 449 | CUGGAGUUGUCAUCACGCU | AGCGUGAUGACAACUCCAG | | [547-565] |
| 450 | AGCACUCUCUGAACUUGGA | UCCAAGUUCAGAGAGUGCU | | [431-449] |
| 451 | UUAACAGGUACUCUAAGAA | UUCUUAGAGUACCUGUUAA | | [4060-4078] |
| 452 | GUGUUAUCCAGUUAUGAGA | UCUCAUAACUGGAUAACAC | | [3900-3918] |
| 453 | GAGAAUAUUGGCCCUUUCU | AGAAAGGGCCAAUAUUCUC | | [3772-3790] |
| 454 | CAAAGUAUGAACUGGAGAG | CUCUCCAGUUCAUACUUUG | | [3730-3748] |
| 455 | AGGGACUAGGAUGAACUAA | UUAGUUCAUCCUAGUCCCU | | [3452-3470] |
| 456 | CAAGGGACUAGGAUGAACU | AGUUCAUCCUAGUCCCUUG | | [3450-3468] |
| 457 | CUUUUAAAAUCUGGUCACA | UGUGACCAGAUUUUAAAAG | | [3406-3424] |
| 458 | UCUUUUAAAAUCUGGUCAC | GUGACCAGAUUUUAAAAGA | | [3405-3423] |
| 459 | AGAAUAUAAUUGAAGGAGG | CCUCCUUCAAUUAUAUUCU | | [3344-3362] |
| 460 | UGGGAGAGUGAGAGAAAAU | AUUUUCUCUCACUCUCCCA | | [3241-3259] |
| 461 | ACUCUUCAACUUCGGUAAU | AUUACCGAAGUUGAAGAGU | | [3146-3164] |
| 462 | GGAACUAAAACCAGUUUUA | UAAAACUGGUUUUAGUUCC | | [3093-3111] |
| 463 | CAGAGAUCAAAUUCACACU | AGUGUGAAUUUGAUCUCUG | | [2969-2987] |
| 464 | UCUGAUAUGGUCAUUUGUU | AACAAAUGACCAUAUCAGA | | [2816-2834] |
| 465 | AACUUAGGAAUUGUGACAA | UUGUCACAAUUCCUAAGUU | | [2790-2808] |
| 466 | GGGUCAAGAACAGGCUAAG | CUUAGCCUGUUCUUGACCC | | [2577-2595] |
| 467 | ACUCAUCAUUUACCACUGU | ACAGUGGUAAAUGAUGAGU | | [2397-2415] |
| 468 | GAAUAUUGGAUUGAUUGGA | UCCAAUCAAUCCAAUAUUC | | [2367-2385] |
| 469 | UGGGCCAGAUGUUCUUUCU | AGAAAGAACAUCUGGCCCA | | [2342-2360] |
| 470 | AACAAGAGUUCGAAGACAA | UUGUCUUCGAACUCUUGUU | | [275-293] |
| 471 | AGUAUUGACAUCUGAGCAU | AUGCUCAGAUGUCAAUACU | | [2294-2312] |
| 472 | UGAAGGAAAUUUUCCAGAU | AUCUGGAAAAUUUCCUUCA | | [2227-2245] |
| 473 | UGUCAUUUUUGCCAUCAUU | AAUGAUGGCAAAAAUGACA | | [2198-2216] |
| 474 | GAAAGGAUCAAAAGGUUCA | UGAACCUUUUGAUCCUUUC | | [2016-2034] |
| 475 | AGGGAAAGGAUCAAAAGGU | ACCUUUUGAUCCUUUCCCU | | [2013-2031] |
| 476 | GAAUCAAAAGGGAAAGGAU | AUCCUUUCCCUUUUGAUUC | | [2005-2023] |
| 477 | AGAAUCAAAAGGGAAAGGA | UCCUUUCCCUUUUGAUUCU | | [2004-2022] |
| 478 | CUUGGACUCAAUUUUAGAA | UUCUAAAAUUGAGUCCAAG | | [1989-2007] |
| 479 | GAUUCUUGGACUCAAUUUU | AAAAUUGAGUCCAAGAAUC | | [1985-2003] |
| 480 | AGUUAGGGAAAAGAUUCUU | AAGAAUCUUUUCCCUAACU | | [1973-1991] |
| 481 | UGGAAGUUAGGGAAAAGAU | AUCUUUUCCCUAACUUCCA | | [1969-1987] |
| 482 | GUGGAAGUUAGGGAAAAGA | UCUUUUCCCUAACUUCCAC | | [1968-1986] |
| 483 | CUCAGUAAUGUCAGUGGAA | UUCCACUGACAUUACUGAG | | [1955-1973] |
| 484 | GAGAGCACUUUUACAAACA | UGUUUGUAAAAGUGCUCUC | | [1918-1936] |
| 485 | UGAGAGCACUUUUACAAAC | GUUUGUAAAAGUGCUCUCA | | [1917-1935] |
| 486 | UGGUUUUGAGAGCACUUUU | AAAAGUGCUCUCAAAACCA | | [1911-1929] |
| 487 | AUGGUUUUGAGAGCACUUU | AAAGUGCUCUCAAAACCAU | | [1910-1928] |
| 488 | GGAAAACUUCUAACUUGUC | GACAAGUUAGAAGUUUUCC | | [1715-1733]+ |
| 489 | AGGAGAAAGAUGUGAUCAC | GUGAUCACAUCUUUCUCCU | Rb, Ms | [1504-1522] |
| 490 | CCAAUCUGAAGCUCAAAAA | UUUUUGAGCUUCAGAUUGG | | [1309-1327] |
| 491 | CAGUCUGGUACAAAUAUUG | CAAUAUUUGUACCAGACUG | Rb | [1279-1297] |
| 492 | GCAUCCAUUCUCAAGUCAG | CUGACUUGAGAAUGGAUGC | | [1263-1281] |
| 493 | UGAAGAUGUGUUCAGCUAU | AUAGCUGAACACAUCUUCA | Rat, Rb, Ms | [1199-1217] |
| 494 | CUGAGGAAGACUUCUUUAG | CUAAAGAAGUCUUCCUCAG | Rat | [1051-1069] |
| 495 | UCUCCAUUUUUGUCAUUCU | AGAAUGACAAAAAUGGAGA | | [43-61] |
| 496 | CUCUCCAUUUUUGUCAUUC | GAAUGACAAAAAUGGAGAG | | [42-60] |
| 497 | GCGAUCUCAACAGAAGGUG | CACCUUCUGUUGAGAUCGC | | [881-899] |
| 498 | UUAUGAUAUUCCACCUAAG | CUUAGGUGGAAUAUCAUAA | | [110-128] |
| 499 | UGAUCUUCUUCAUUGGCCU | AGGCCAAUGAAGAAGAUCA | | [652-670] |
| 500 | GAGUUGUCAUCACGCUGUG | CACAGCGUGAUGACAACUC | | [550-568] |
| 501 | AGGCAUCACUGGAGUUGUC | GACAACUCCAGUGAUGCCU | | [539-557] |
| 502 | UCCUUAUUCAGUAGCACUC | GAGUGCUACUGAAUAAGGA | | [419-437] |
| 503 | UAAGAAUGAUGAAGCAUUG | CAAUGCUUCAUCAUUCUUA | | [4073-4091] |
| 504 | AGGUACUCUAAGAAUGAUG | CAUCAUUCUUAGAGUACCU | | [4065-4083] |
| 505 | UCCAAGGUUUUCUAUCUUC | GAAGAUAGAAAACCUUGGA | | [3843-3861] |
| 506 | CACACCAUUGCACAUCUAU | AUAGAUGUGCAAUGGUGUG | | [357-375] |
| 507 | CCUUUCGUCUCCCAGAAUC | GAUUCUGGGAGACGAAAGG | | [3506-3524] |
| 508 | UCAAGGGACUAGGAUGAAC | GUUCAUCCUAGUCCCUUGA | | [3449-3467] |
| 509 | AUGGGAGAGUGAGAGAAAA | UUUUCUCUCACUCUCCCAU | | [3240-3258] |
| 510 | AGGAAAAGGAAAUAAGGAG | CUCCUUAUUUCCUUUUCCU | | [3213-3231] |
| 511 | AGGAGGAAAAGGAAAUAAG | CUUAUUUCCUUUUCCUCCU | | [3210-3228] |
| 512 | CUGUUAGUGUGGCCAAGAG | CUCUUGGCCACACUAACAG | | [3034-3052] |
| 513 | UCCAGCUAGCCCUUAUGAA | UUCAUAAGGGCUAGCUGGA | | [2766-2784] |
| 514 | UCUGCACAUUCUUAUUUGA | UCAAAUAAGAAUGUGCAGA | | [2626-2644] |
| 515 | GAACAGGCUAAGGAUACUA | UAGUAUCCUUAGCCUGUUC | | [2584-2602] |
| 516 | AGGAAAUUUUCCAGAUCAU | AUGAUCUGGAAAAUUUCCU | | [2230-2248] |
| 517 | GAAGGAAAUUUUCCAGAUC | GAUCUGGAAAAUUUCCUUC | | [2228-2246] |
| 518 | AGAAGAAGGAAACCAAGGA | UCCUUGGUUUCCUUCUUCU | | [2157-2175] |
| 519 | ACUUCCCUAAGAUUAUGAA | UUCAUAAUCUUAGGGAAGU | | [2038-2056] |
| 520 | GGAAAGGAUCAAAAGGUUC | GAACCUUUUGAUCCUUUCC | | [2015-2033] |
| 521 | GUAAUGUCAGUGGAAGUUA | UAACUUCCACUGACAUUAC | | [1959-1977] |
| 522 | UCAGUAAUGUCAGUGGAAG | CUUCCACUGACAUUACUGA | | [1956-1974] |
| 523 | CAAAGAUUGUUUGAUAGUG | CACUAUCAAACAAUCUUUG | | [1866-1884] |
| 524 | AGGAAUGUCAAAGAUUGUU | AACAAUCUUUGACAUUCCU | | [1858-1876] |
| 525 | GAAAUAAAUGUGGGUUGUG | CACAACCCACAUUUAUUUC | | [1745-1763] |
| 526 | GAAAACUUCUAACUUGUCU | AGACAAGUUAGAAGUUUUC | | [1716-1734]+ |
| 527 | CUGAAACAAAAGACUUUGU | ACAAAGUCUUUUGUUUCAG | | [1527-1545] |
| 528 | CCUGAAACAAAAGACUUUG | CAAAGUCUUUUGUUUCAGG | | [1526-1544] |
| 529 | GCUACAACAUCUACCUCAC | GUGAGGUAGAUGUUGUAGC | | [1438-1456] |
| 530 | GAUGCAGGAAAGGAACAAU | AUUGUUCCUUUCCUGCAUC | Ms | [1406-1424] |
| 531 | CCUUCGCAUCCAUUCUCAA | UUGAGAAUGGAUGCGAAGG | | [1258-1276] |
| 532 | UGAUAAGCAGGAGUUUCAA | UUGAAACUCCUGCUUAUCA | Rat, Rb | [1127-1145] |
| 533 | GUAUCCAUAUCCGCAUCGU | ACGAUGCGGAUAUGGAUAC | | [1069-1087] |
| 534 | UGAGGAAGACUUCUUUAGU | ACUAAAGAAGUCUUCCUCA | | [1052-1070] |
| 535 | CCCUGAGGAAGACUUCUUU | AAAGAAGUCUUCCUCAGGG | Rat | [1049-1067] |
| 536 | UGGCACCCUUUUACACUGA | UCAGUGUAAAAGGGUGCCA | | [1023-1041] |
| 537 | UGACUUGGAAAUGGAUAGU | ACUAUCCAUUUCCAAGUCA | Ms | [817-835] |
| 538 | CUAUGACUUGGAAAUGGAU | AUCCAUUUCCAAGUCAUAG | Rat, Ms | [814-832] |
| 539 | UGGAGACAGGCAAAAUGAA | UUCAUUUUGCCUGUCUCCA | | [446-464] |
| 540 | CUUCCUCUUUGUCUGGUAU | AUACCAGACAAAGAGGAAG | | [83-101] |
| 541 | UCUUCCUCUUUGUCUGGUA | UACCAGACAAAGAGGAAGA | | [82-100] |
| 542 | GUAGCACUCUCUGAACUUG | CAAGUUCAGAGAGUGCUAC | | [429-447] |
| 543 | ACGUCUUCCUCUUUGUCUG | CAGACAAAGAGGAAGACGU | | [79-97] |
| 544 | GACCUGGGAGAUAAUUCUA | UAGAAUUAUCUCCCAGGUC | | [4200-4218] |
| 545 | GAAAUUUGAAUACUUGCAG | CUGCAAGUAUUCAAAUUUC | | [4153-4171] |
| 546 | GGAAUGGUGUGUGAAUGCC | GGCAUUCACACACCAUUCC | | [383-401] |
| 547 | GUGGAAUGGUGUGUGAAUG | CAUUCACACACCAUUCCAC | | [381-399] |
| 548 | GUUUCUCCAAGGUUUUCUA | UAGAAAACCUUGGAGAAAC | | [3838-3856] |
| 549 | GUGCGAAUAUAGCCUUUCU | AGAAAGGCUAUAUUCGCAC | | [3565-3583] |
| 550 | UAGGAGGAAAAGGAAAUAA | UUAUUUCCUUUUCCUCCUA | | [3209-3227] |
| 551 | UUAGGAGGAAAAGGAAAUA | UAUUUCCUUUUCCUCCUAA | | [3208-3226] |
| 552 | GGUUUAGGAGGAAAAGGAA | UUCCUUUUCCUCCUAAACC | | [3205-3223] |
| 553 | UGGUUUAGGAGGAAAAGGA | UCCUUUUCCUCCUAAACCA | | [3204-3222] |
| 554 | CCUUAUUUUCCGUAAAUAC | GUAUUUACGGAAAAUAAGG | | [2851-2869] |
| 555 | GGUUCUGCACAUUCUUAUU | AAUAAGAAUGUGCAGAACC | | [2623-2641] |
| 556 | GGUAACUGGGCCAGAUGUU | AACAUCUGGCCCAGUUACC | | [2336-2354] |
| 557 | GCAUACUCCAGUUUACUAA | UUAGUAAACUGGAGUAUGC | | [2309-2327] |
| 558 | GUCAUUUUUGCCAUCAUUA | UAAUGAUGGCAAAAAUGAC | | [2199-2217] |
| 559 | GAAGAAGGAAACCAAGGAG | CUCCUUGGUUUCCUUCUUC | | [2158-2176] |
| 560 | AGGAAAACUUCUAACUUGU | ACAAGUUAGAAGUUUUCCU | | [1714-1732]+ |
| 561 | ACAAAAGACUUUGUAUGGA | UCCAUACAAAGUCUUUUGU | | [1532-1550] |
| 562 | AGCUACAACAUCUACCUCA | UGAGGUAGAUGUUGUAGCU | | [1437-1455] |
| 563 | GGUGAUGUUAGUGGGAGCA | UGCUCCCACUAACAUCACC | Rb | [1223-1241] |
| 564 | CUAUGAGGUGGUGAUGUUA | UAACAUCACCACCUCAUAG | Rat, Rb, Ms | [1214-1232] |
| 565 | UGCCAGUGAAGAUGUGUUC | GAACACAUCUUCACUGGCA | | [1193-1211] |
| 566 | UUAGUAUCCAUAUCCGCAU | AUGCGGAUAUGGAUACUAA | | [1066-1084] |
| 567 | CUUCUUUAGUAUCCAUAUC | GAUAUGGAUACUAAAGAAG | | [1061-1079] |
| 568 | CCCUUUUACACUGACAUCC | GGAUGUCAGUGUAAAAGGG | | [1028-1046] |
| 569 | GUGGCACCCUUUUACACUG | CAGUGUAAAAGGGUGCCAC | | [1022-1040] |
| 570 | GACUUGGAAAUGGAUAGUG | CACUAUCCAUUUCCAAGUC | Ms | [818-836] |
| 571 | GUAUUACCGGGUUUAUGAU | AUCAUAAACCCGGUAAUAC | | [98-116] |
| 572 | GGCAUCACUGGAGUUGUCA | UGACAACUCCAGUGAUGCC | | [540-558] |
| 573 | CCUCUUUGUCUGGUAUUAC | GUAAUACCAGACAAAGAGG | | [86-104] |
| 574 | GUCUUCCUCUUUGUCUGGU | ACCAGACAAAGAGGAAGAC | | [81-99] |
| 575 | ACCUGGGAGAUAAUUCUAC | GUAGAAUUAUCUCCCAGGU | | [4201-4219] |
| 576 | GGGACCUGGGAGAUAAUUC | GAAUUAUCUCCCAGGUCCC | | [4198-4216] |
| 577 | UUGAAUACUUGCAGGCUUU | AAAGCCUGCAAGUAUUCAA | | [4158-4176] |
| 578 | ACAGGUACUCUAAGAAUGA | UCAUUCUUAGAGUACCUGU | | [4063-4081] |
| 579 | UCCUUUUUAUCUCUCAAGC | GCUUGAGAGAUAAAAAGGA | | [3958-3976] |
| 580 | UUCCUUUUUAUCUCUCAAG | CUUGAGAGAUAAAAAGGAA | | [3957-3975] |
| 581 | GUUUCCUUUUUAUCUCUCA | UGAGAGAUAAAAAGGAAAC | | [3955-3973] |
| 582 | CUGUGCGAAUAUAGCCUUU | AAAGGCUAUAUUCGCACAG | | [3563-3581] |
| 583 | GGAGGAAAAGGAAAUAAGG | CCUUAUUUCCUUUUCCUCC | | [3211-3229] |
| 584 | GGGAACUAAAACCAGUUUU | AAAACUGGUUUUAGUUCCC | | [3092-3110] |
| 585 | AGGGUUGGAGGUGGAGAAU | AUUCUCCACCUCCAACCCU | | [2658-2676] |
| 586 | AGGGUUCUGCACAUUCUUA | UAAGAAUGUGCAGAACCCU | | [2621-2639] |
| 587 | GGAUUGCCUGAAGGGUUCU | AGAACCCUUCAGGCAAUCC | | [2610-2628] |
| 588 | UCAAGAACAGGCUAAGGAU | AUCCUUAGCCUGUUCUUGA | | [2580-2598] |
| 589 | GUCAAGAACAGGCUAAGGA | UCCUUAGCCUGUUCUUGAC | | [2579-2597] |
| 590 | CUAGGGUCAAGAACAGGCU | AGCCUGUUCUUGACCCUAG | | [2574-2592] |
| 591 | CUGGGCCAGAUGUUCUUUC | GAAAGAACAUCUGGCCCAG | | [2341-2359] |
| 592 | CUCCAGUUUACUAAUACAG | CUGUAUUAGUAAACUGGAG | | [2314-2332] |
| 593 | GGACAUAAUACAUGUUGAG | CUCAACAUGUAUUAUGUCC | | [2251-2269] |
| 594 | GGAAAUUUUCCAGAUCAUU | AAUGAUCUGGAAAAUUUCC | | [2231-2249] |
| 595 | CUACUUUGUGUCAUUUUUG | CAAAAAUGACACAAAGUAG | | [2190-2208] |
| 596 | AGUAAUGUCAGUGGAAGUU | AACUUCCACUGACAUUACU | | [1958-1976] |
| 597 | CUUAAAGGAAUGUCAAAGA | UCUUUGACAUUCCUUUAAG | | [1853-1871] |
| 598 | CCUGAAGCCUUGGCUGAAA | UUUCAGCCAAGGCUUCAGG | Rb, Ms | [1629-1647] |
| 599 | UGCAAGUCAACACCCUAAU | AUUAGGGUGUUGACUUGCA | Rb | [1583-1601] |
| 600 | CAAAAGACUUUGUAUGGAC | GUCCAUACAAAGUCUUUUG | | [1533-1551] |
| 601 | CUACAACAUCUACCUCACU | AGUGAGGUAGAUGUUGUAG | | [1439-1457] |
| 602 | GGGAAAAAUAAAGGAAUGC | GCAUUCCUUUAUUUUUCCC | | [767-785] |
| 603 | UGGAGCUGAACGAAUUGUA | UACAAUUCGUUCAGCUCCA | Rat, Rb, Ms | [680-698] |
| 604 | UCUCCAAGGUUUUCUAUCU | AGAUAGAAAACCUUGGAGA | | [3841-3859] |
| 605 | GCUGUGCGAAUAUAGCCUU | AAGGCUAUAUUCGCACAGC | | [3562-3580] |
| 606 | GAAGACUGGUUUAGGAGGA | UCCUCCUAAACCAGUCUUC | | [3198-3216] |
| 607 | UUCUCCAGCUAGCCCUUAU | AUAAGGGCUAGCUGGAGAA | | [2763-2781] |
| 608 | CCUGCUCCUUGUGCUAUAA | UUAUAGCACAAGGAGCAGG | | [2709-2727] |
| 609 | CCAAUAGGAUUGCCUGAAG | CUUCAGGCAAUCCUAUUGG | | [2604-2622] |
| 610 | AGAACAGGCUAAGGAUACU | AGUAUCCUUAGCCUGUUCU | | [2583-2601] |
| 611 | ACUCCAGUUUACUAAUACA | UGUAUUAGUAAACUGGAGU | | [2313-2331] |
| 612 | CAGAUCAUUAGGACAUAAU | AUUAUGUCCUAAUGAUCUG | | [2241-2259] |
| 613 | AAGGAAAUUUUCCAGAUCA | UGAUCUGGAAAAUUUCCUU | | [2229-2247] |
| 614 | GAAUGUCAAAGAUUGUUUG | CAAACAAUCUUUGACAUUC | | [1860-1878] |
| 615 | ACUUCUAACUUGUCUCUUC | GAAGAGACAAGUUAGAAGU | | [1720-1738]+ |
| 616 | AAGGAAAACUUCUAACUUG | CAAGUUAGAAGUUUUCCUU | | [1713-1731]+ |
| 617 | CCAAUCACUUUGCUGUGCA | UGCACAGCAAAGUGAUUGG | Rat, Rb, Ms | [1477-1495] |
| 618 | GUGGUGAUGUUAGUGGGAG | CUCCCACUAACAUCACCAC | Rat, Rb, Ms | [1221-1239] |
| 619 | GUGGGUCCCAUGUUUCUGU | ACAGAAACAUGGGACCCAC | | [834-852] |
| 620 | UGUCUGGUAUUACCGGGUU | AACCCGGUAAUACCAGACA | | [92-110] |
| 621 | CCCUGUGUUAUCCAGUUAU | AUAACUGGAUAACACAGGG | | [3896-3914] |
| 622 | GCCCUGUGUUAUCCAGUUA | UAACUGGAUAACACAGGGC | | [3895-3913] |
| 623 | AAGACUGGUUUAGGAGGAA | UUCCUCCUAAACCAGUCUU | | [3199-3217] |
| 624 | GGAAGAAGGAGAAAGAGCU | AGCUCUUUCUCCUUCUUCC | | [3167-3185] |
| 625 | AUCAUCAUCAUCAUCCUAA | UUAGGAUGAUGAUGAUGAU | | [2919-2937] |
| 626 | AACAGGCUAAGGAUACUAA | UUAGUAUCCUUAGCCUGUU | | [2585-2603] |
| 627 | CCAGUUUACUAAUACAGCA | UGCUGUAUUAGUAAACUGG | | [2316-2334] |
| 628 | UCCAGAUCAUUAGGACAUA | UAUGUCCUAAUGAUCUGGA | | [2239-2257] |
| 629 | UGUGUCAUUUUUGCCAUCA | UGAUGGCAAAAAUGACACA | | [2196-2214] |
| 630 | UCAUUUUUUUCCUCUCAGU | ACUGAGAGGAAAAAAAUGA | | [1942-1960] |
| 631 | CCCUGAGUAAACAAAGCAU | AUGCUUUGUUUACUCAGGG | | [1648-1666] |
| 632 | GGGACACACAUGCCUUUGA | UCAAAGGCAUGUGUGUCCC | | [1351-1369] |
| 633 | UCAAGUCAGUCUGGUACAA | UUGUACCAGACUGACUUGA | Rb | [1273-1291] |
| 634 | CGUGGAAACUACCUAAGAU | AUCUUAGGUAGUUUCCACG | | [1150-1168] |
| 635 | CAAUGCUUGUGGCUGUGAU | AUCACAGCCACAAGCAUUG | Rb, Ms | [1112-1130] |
| 636 | CCAUCGAGCUACAGAUGAA | UUCAUCUGUAGCUCGAUGG | | [934-952] |
| 637 | GGAGCUGAACGAAUUGUAC | GUACAAUUCGUUCAGCUCC | Rat, Rb, Ms | [681-699] |
| 638 | UUCUUCUGCUUUUGCAAUU | AAUUGCAAAAGCAGAAGAA | | [4126-4144] |
| 639 | UCGCAUCUGUGUGACUAAU | AUUAGUCACACAGAUGCGA | | [2884-2902] |
| 640 | UCCUGCCCAAAGUGGUCUU | AAGACCACUUUGGGCAGGA | | [2746-2764] |
| 641 | AGACUAAUUUCCUGCCCAA | UUGGGCAGGAAAUUAGUCU | | [2737-2755] |
| 642 | CUGCUCCUUGUGCUAUAAA | UUUAUAGCACAAGGAGCAG | | [2710-2728] |
| 643 | UCCUGCUCCUUGUGCUAUA | UAUAGCACAAGGAGCAGGA | | [2708-2726] |
| 644 | ACACCCUAAUACCAGAAUA | UAUUCUGGUAUUAGGGUGU | Rb | [1592-1610] |
| 645 | GUACCUCAGUUAUAAGGAG | CUCCUUAUAACUGAGGUAC | | [3750-3768] |
| 646 | GGAGAGGGUACCUCAGUUA | UAACUGAGGUACCCUCUCC | | [3743-3761] |
| 647 | UGAACGUCUUCCUCUUUGU | ACAAAGAGGAAGACGUUCA | Rb | [76-94] |
| 648 | GAGGUAUACACAUACUGAU | AUCAGUAUGUGUAUACCUC | | [3359-3377] |
| 649 | CAAGAGAGAGUCUCUAAAU | AUUUAGAGACUCUCUCUUG | | [3013-3031] |
| 650 | UCAAGAGAGAGUCUCUAAA | UUUAGAGACUCUCUCUUGA | | [3012-3030] |
| 651 | CUGAGCAUACUCCAGUUUA | UAAACUGGAGUAUGCUCAG | | [2305-2323] |
| 652 | GGCUGAAACCCUGAGUAAA | UUUACUCAGGGUUUCAGCC | | [1640-1658] |
| 653 | UUGGCUGAAACCCUGAGUA | UACUCAGGGUUUCAGCCAA | | [1638-1656] |
| 654 | GGCCCAACUGGGAUAAUGA | UCAUUAUCCCAGUUGGGCC | | [1552-1570] |
| 655 | UCACACCCUUCGCAUCCAU | AUGGAUGCGAAGGGUGUGA | | [1252-1270] |
| 656 | GUGGAAACUACCUAAGAUA | UAUCUUAGGUAGUUUCCAC | | [1151-1169] |
| 657 | GGACAAUACAUUUUUGUCA | UGACAAAAAUGUAUUGUCC | | [978-996] |
| 658 | AUCAUCUCUUUGUGAUCUU | AAGAUCACAAAGAGAUGAU | | [640-658] |
| 659 | CUGAGAAUAUUGGCCCUUU | AAAGGGCCAAUAUUCUCAG | | [3770-3788] |
| 660 | CGCAUCUGUGUGACUAAUG | CAUUAGUCACACAGAUGCG | | [2885-2903] |
| 661 | CGAAGACAACUGGACAGGA | UCCUGUCCAGUUGUCUUCG | Rat, Ms | [285-303] |
| 662 | AGGACAUAAUACAUGUUGA | UCAACAUGUAUUAUGUCCU | | [2250-2268] |
| 663 | UGAUACUCUUUCCACAAUA | UAUUGUGGAAAGAGUAUCA | | [2092-2110] |
| 664 | CUGAAGCCUUGGCUGAAAC | GUUUCAGCCAAGGCUUCAG | Rb, Ms | [1630-1648] |
| 665 | GCCCAACUGGGAUAAUGAA | UUCAUUAUCCCAGUUGGGC | | [1553-1571] |
| 666 | CCUGCCUGAAUUUCAACUG | CAGUUGAAAUUCAGGCAGG | Rat, Ms | [187-205] |
| 667 | CUGGUACAAAUAUUGCAAU | AUUGCAAUAUUUGUACCAG | | [1283-1301] |
| 668 | GCGUGGAAACUACCUAAGA | UCUUAGGUAGUUUCCACGC | | [1149-1167] |
| 669 | AGAAGGUGGUCAUCACCAA | UUGGUGAUGACCACCUUCU | Rb | [892-910] |
| 670 | CGAAUUGUACGUGGGCAGA | UCUGCCCACGUACAAUUCG | | [690-708] |
| 671 | ACGAAUUGUACGUGGGCAG | CUGCCCACGUACAAUUCGU | | [689-707] |
| 672 | AGCUGAACGAAUUGUACGU | ACGUACAAUUCGUUCAGCU | Rat, Rb, Ms | [683-701] |
| 673 | CGAGUCAAUAAUUCUGAUC | GAUCAGAAUUAUUGACUCG | | [402-420] |
| 674 | CUUCUGCUUUUGCAAUUGU | ACAAUUGCAAAAGCAGAAG | | [4128-4146] |
| 675 | CUAUGAGUCAGGCUCUGUU | AACAGAGCCUGACUCAUAG | | [3690-3708] |
| 676 | UGCGAAUAUAGCCUUUCUG | CAGAAAGGCUAUAUUCGCA | | [3566-3584] |
| 677 | GGAGGUAUACACAUACUGA | UCAGUAUGUGUAUACCUCC | | [3358-3376] |
| 678 | CCAUUUCUGGGAACUAAAA | UUUUAGUUCCCAGAAAUGG | | [3084-3102] |
| 679 | UGCCCAAAGUGGUCUUCUC | GAGAAGACCACUUUGGGCA | | [2749-2767] |
| 680 | UGCUCCUUGUGCUAUAAAA | UUUUAUAGCACAAGGAGCA | | [2711-2729] |
| 681 | CCAUGACUCUGGCUCAUCU | AGAUGAGCCAGAGUCAUGG | | [2687-2705] |
| 682 | ACCAAUAGGAUUGCCUGAA | UUCAGGCAAUCCUAUUGGU | | [2603-2621] |
| 683 | GCCAGAUGUUCUUUCUACA | UGUAGAAAGAACAUCUGGC | | [2345-2363] |
| 684 | AUCUGAGCAUACUCCAGUU | AACUGGAGUAUGCUCAGAU | | [2303-2321] |
| 685 | GAAAUCUGCUGUCCUUCCU | AGGAAGGACAGCAGAUUUC | | [232-250] |
| 686 | UGUAAUGGUUUUCCCUUAA | UUAAGGGAAAACCAUUACA | | [1839-1857] |
| 687 | UGGCUGAAACCCUGAGUAA | UUACUCAGGGUUUCAGCCA | | [1639-1657] |
| 688 | CAAGUCAACACCCUAAUAC | GUAUUAGGGUGUUGACUUG | Rb | [1585-1603] |
| 689 | CCCAACUGGGAUAAUGAAU | AUUCAUUAUCCCAGUUGGG | | [1554-1572] |
| 690 | ACGGCCCAACUGGGAUAAU | AUUAUCCCAGUUGGGCCGU | | [1550-1568] |
| 691 | CAAUCACUUUGCUGUGCAC | GUGCACAGCAAAGUGAUUG | Rat, Rb, Ms | [1478-1496] |
| 692 | CUGCAGCCUGCCUGAAUUU | AAAUUCAGGCAGGCUGCAG | Rat, Ms | [181-199] |
| 693 | UGCGUGGAAACUACCUAAG | CUUAGGUAGUUUCCACGCA | | [1148-1166] |
| 694 | UCAAAACCAUCGAGCUACA | UGUAGCUCGAUGGUUUUGA | | [928-946] |
| 695 | UCAGUUUGCUGGAAACCCU | AGGGUUUCCAGCAAACUGA | | [794-812] |
| 696 | UUGCUCGAAAGAGAAUAAA | UUUAUUCUCUUUCGAGCAA | | [478-496] |
| 697 | UGCCCUGUGUUAUCCAGUU | AACUGGAUAACACAGGGCA | | [3894-3912] |
| 698 | UGCAUAAUUAAAACCAGCU | AGCUGGUUUUAAUUAUGCA | | [3799-3817] |
| 699 | GGCCCUUUCUAACCUAUGU | ACAUAGGUUAGAAAGGGCC | | [3781-3799] |
| 700 | CAAUUCUUUACACCAAAGU | ACUUUGGUGUAAAGAAUUG | | [3717-3735] |
| 701 | UCACAAAUUUUGAUGCUGA | UCAGCAUCAAAAUUUGUGA | | [3420-3438] |
| 702 | UCAUAUUGGUCAUUCCCUG | CAGGGAAUGACCAAUAUGA | | [3296-3314] |
| 703 | CCCAUUUCUGGGAACUAAA | UUUAGUUCCCAGAAAUGGG | | [3083-3101] |
| 704 | UCUUAGAACUGCUCCCAUU | AAUGGGAGCAGUUCUAAGA | | [3070-3088] |
| 705 | CCAGAAACAAAAAUCUCUA | UAGAGAUUUUUGUUUCUGG | | [2948-2966] |
| 706 | GUGACAAAUAUGUAUCUGA | UCAGAUACAUAUUUGUCAC | | [2802-2820] |
| 707 | UCACCUUUCAUAAAAUGGU | ACCAUUUUAUGAAAGGUGA | | [307-325] |
| 708 | AGGAAUCUCACCUUUCAUA | UAUGAAAGGUGAGAUUCCU | | [300-318] |
| 709 | GGAUACUAACCAAUAGGAU | AUCCUAUUGGUUAGUAUCC | | [2595-2613] |
| 710 | GGAUACUCAAGUAAGUUUU | AAAACUUACUUGAGUAUCC | | [2430-2448] |
| 711 | UGAGCAUACUCCAGUUUAC | GUAAACUGGAGUAUGCUCA | | [2306-2324] |
| 712 | GAGAGUGUCUCAACACUUA | UAAGUGUUGAGACACUCUC | | [2267-2285] |
| 713 | UUCCAGAUCAUUAGGACAU | AUGUCCUAAUGAUCUGGAA | | [2238-2256] |
| 714 | UCAGUAACUUCCCUAAGAU | AUCUUAGGGAAGUUACUGA | | [2032-2050] |
| 715 | UUCAUUUUUUUCCUCUCAG | CUGAGAGGAAAAAAAUGAA | | [1941-1959] |
| 716 | GCAUCUCCAACUCUGAGUC | GACUCAGAGUUGGAGAUGC | Rat, Rb | [1663-1681] |
| 717 | AUACCAGAAUAGGAGUUUU | AAAACUCCUAUUCUGGUAU | Rb | [1600-1618] |
| 718 | UAAUACCAGAAUAGGAGUU | AACUCCUAUUCUGGUAUUA | Rb | [1598-1616] |
| 719 | UGCACCAUGAUGAGGAGAA | UUCUCCUCAUCAUGGUGCA | Rb, Ms | [1492-1510] |
| 720 | AGGCCAAUCACUUUGCUGU | ACAGCAAAGUGAUUGGCCU | Rat, Rb, Ms | [1474-1492] |
| 721 | ACCUAAGAUAGCGGUUGAU | AUCAACCGCUAUCUUAGGU | | [1160-1178] |
| 722 | CUACCUAAGAUAGCGGUUG | CAACCGCUAUCUUAGGUAG | | [1158-1176] |
| 723 | GUGAUAAGCAGGAGUUUCA | UGAAACUCCUGCUUAUCAC | Rat, Rb | [1126-1144] |
| 724 | CUGUGAUAAGCAGGAGUUU | AAACUCCUGCUUAUCACAG | Rat, Rb | [1124-1142] |
| 725 | CUGGAGUGGCACCCUUUUA | UAAAAGGGUGCCACUCCAG | | [1017-1035] |
| 726 | UCCCUCAGUUUGCUGGAAA | UUUCCAGCAAACUGAGGGA | | [790-808] |
| 727 | ACCAAAACCAUCCGGAGGU | ACCUCCGGAUGGUUUUGGU | | [594-612] |
| 728 | GCAAAAUGAAAGUUAUCUC | GAGAUAACUUUCAUUUUGC | | [455-473] |
| 729 | UCAGUUAUAAGGAGUCUGA | UCAGACUCCUUAUAACUGA | | [3755-3773] |
| 730 | CCUCAGUUAUAAGGAGUCU | AGACUCCUUAUAACUGAGG | | [3753-3771] |
| 731 | CUUUACACCAAAGUAUGAA | UUCAUACUUUGGUGUAAAG | | [3722-3740] |
| 732 | CUAAAUAAGAACUCAGUUG | CAACUGAGUUCUUAUUUAG | | [3467-3485] |
| 733 | ACACAUACUGAUGUUGUUU | AAACAACAUCAGUAUGUGU | | [3366-3384] |
| 734 | GUAUACACAUACUGAUGUU | AACAUCAGUAUGUGUAUAC | | [3362-3380] |
| 735 | UCCCAUUUCUGGGAACUAA | UUAGUUCCCAGAAAUGGGA | | [3082-3100] |
| 736 | GCAGACUAAUUUCCUGCCC | GGGCAGGAAAUUAGUCUGC | | [2735-2753] |
| 737 | UGCCAUGACUCUGGCUCAU | AUGAGCCAGAGUCAUGGCA | | [2685-2703] |
| 738 | GAAUUAACCUCCUGCCAUG | CAUGGCAGGAGGUUAAUUC | | [2673-2691] |
| 739 | UGGACAGGAAUCUCACCUU | AAGGUGAGAUUCCUGUCCA | | [295-313] |
| 740 | CUGUCAUAGACCAAAGGAA | UUCCUUUGGUCUAUGACAG | | [2544-2562] |
| 741 | CGGAUACUCAAGUAAGUUU | AAACUUACUUGAGUAUCCG | | [2429-2447] |
| 742 | GGAGUUAAUGUAAUACUCA | UGAGUAUUACAUUAACUCC | | [2383-2401] |
| 743 | AGCAUACUCCAGUUUACUA | UAGUAAACUGGAGUAUGCU | | [2308-2326] |
| 744 | CUGAAGGAAAUUUUCCAGA | UCUGGAAAAUUUCCUUCAG | | [2226-2244] |
| 745 | CCCAAAAGAAGAAGGAAAC | GUUUCCUUCUUCUUUUGGG | | [2151-2169] |
| 746 | AUACUGAGCUGCCUCAGAA | UUCUGAGGCAGCUCAGUAU | | [2108-2126] |
| 747 | GCCCAUCUUACUCCAGGUU | AACCUGGAGUAAGAUGGGC | | [2073-2091] |
| 748 | GGACUCAAUUUUAGAAUCA | UGAUUCUAAAAUUGAGUCC | | [1992-2010] |
| 749 | AUGUAAUGGUUUUCCCUUA | UAAGGGAAAACCAUUACAU | | [1838-1856] |
| 750 | CACCCUAAUACCAGAAUAG | CUAUUCUGGUAUUAGGGUG | Rb | [1593-1611] |
| 751 | AACACCCUAAUACCAGAAU | AUUCUGGUAUUAGGGUGUU | Rb | [1591-1609] |
| 752 | UCACUUUGCUGUGCACCAU | AUGGUGCACAGCAAAGUGA | Rb, Ms | [1481-1499] |
| 753 | AGAGCCAGAUGCAGGAAAG | CUUUCCUGCAUCUGGCUCU | | [1399-1417] |
| 754 | GAGAGCCAGAUGCAGGAAA | UUUCCUGCAUCUGGCUCUC | | [1398-1416] |
| 755 | UUGCUGGAAACCCUCCUAU | AUAGGAGGGUUUCCAGCAA | | [799-817] |
| 756 | CCCUCAGUUUGCUGGAAAC | GUUUCCAGCAAACUGAGGG | | [791-809] |
| 757 | AAAUAAAGGAAUGCCCAAU | AUUGGGCAUUCCUUUAUUU | | [772-790] |
| 758 | GUCUGGUAUUACCGGGUUU | AAACCCGGUAAUACCAGAC | | [93-111] |
| 759 | ACCAUCCGGAGGUCUUACU | AGUAAGACCUCCGGAUGGU | | [600-618] |
| 760 | UGCUUUUGCAAUUGUGUUU | AAACACAAUUGCAAAAGCA | | [4132-4150] |
| 761 | CCGAGAGUGUUUCUCCAAG | CUUGGAGAAACACUCUCGG | | [3830-3848] |
| 762 | UUGGCCCUUUCUAACCUAU | AUAGGUUAGAAAGGGCCAA | | [3779-3797] |
| 763 | UCUGAGAAUAUUGGCCCUU | AAGGGCCAAUAUUCUCAGA | | [3769-3787] |
| 764 | ACCUCAGUUAUAAGGAGUC | GACUCCUUAUAACUGAGGU | | [3752-3770] |
| 765 | GCACAUCUAUUUAAUGUGG | CCACAUUAAAUAGAUGUGC | | [366-384] |
| 766 | CUGACAAAUAAGGCCUGCU | AGCAGGCCUUAUUUGUCAG | | [3546-3564] |
| 767 | GAACUAAAUAAGAACUCAG | CUGAGUUCUUAUUUAGUUC | | [3464-3482] |
| 768 | CACAUACUGAUGUUGUUUU | AAAACAACAUCAGUAUGUG | | [3367-3385] |
| 769 | AGGAGGUAUACACAUACUG | CAGUAUGUGUAUACCUCCU | | [3357-3375] |
| 770 | GCCAAGAGCAGGGUUUUCU | AGAAAACCCUGCUCUUGGC | | [3045-3063] |
| 771 | CACCUUUCAUAAAAUGGUG | CACCAUUUUAUGAAAGGUG | | [308-326] |
| 772 | UCUCACCUUUCAUAAAAUG | CAUUUUAUGAAAGGUGAGA | | [305-323] |
| 773 | CAGACUAAUUUCCUGCCCA | UGGGCAGGAAAUUAGUCUG | | [2736-2754] |
| 774 | GUGGAGAAUUAACCUCCUG | CAGGAGGUUAAUUCUCCAC | | [2668-2686] |
| 775 | GCUAAGGAUACUAACCAAU | AUUGGUUAGUAUCCUUAGC | | [2590-2608] |
| 776 | UGGAGUUAAUGUAAUACUC | GAGUAUUACAUUAACUCCA | | [2382-2400] |
| 777 | CAGUUUACUAAUACAGCAG | CUGCUGUAUUAGUAAACUG | | [2317-2335] |
| 778 | UCUGAGCAUACUCCAGUUU | AAACUGGAGUAUGCUCAGA | | [2304-2322] |
| 779 | ACCCUGAGUAAACAAAGCA | UGCUUUGUUUACUCAGGGU | | [1647-1665] |
| 780 | GCCUGAAACAAAAGACUUU | AAAGUCUUUUGUUUCAGGC | | [1525-1543] |
| 781 | GCCUGCCUGAAUUUCAACU | AGUUGAAAUUCAGGCAGGC | Rat, Ms | [186-204] |
| 782 | ACCAAUCUGAAGCUCAAAA | UUUUGAGCUUCAGAUUGGU | | [1308-1326] |
| 783 | GGGCUGUUCAAUGCUUGUG | CACAAGCAUUGAACAGCCC | Rb | [1104-1122] |
| 784 | CCCUUUCAAAACCAUCGAG | CUCGAUGGUUUUGAAAGGG | | [923-941] |
| 785 | GGAUAGUGGGUCCCAUGUU | AACAUGGGACCCACUAUCC | Rb | [829-847] |
| 786 | GCUGUGCAUAAUAUAACAG | CUGUUAUAUUAUGCACAGC | | [723-741] |
| 787 | UCACGCUGUGCCUCAUAUU | AAUAUGAGGCACAGCGUGA | | [559-577] |
| 788 | GAGGGUACCUCAGUUAUAA | UUAUAACUGAGGUACCCUC | | [3746-3764] |
| 789 | ACACUUAGGUCCAGCCUGU | ACAGGCUGGACCUAAGUGU | | [3614-3632] |
| 790 | UGACAAAUAAGGCCUGCUG | CAGCAGGCCUUAUUUGUCA | | [3547-3565] |
| 791 | GUCUGACAAAUAAGGCCUG | CAGGCCUUAUUUGUCAGAC | | [3544-3562] |
| 792 | GUCACAAAUUUUGAUGCUG | CAGCAUCAAAAUUUGUGAC | | [3419-3437] |
| 793 | GGAUGAUUGCACUUCACUC | GAGUGAAGUGCAAUCAUCC | | [331-349] |
| 794 | GCAUGGAUGAUUGCACUUC | GAAGUGCAAUCAUCCAUGC | | [327-345] |
| 795 | UGGGAACUAAAACCAGUUU | AAACUGGUUUUAGUUCCCA | | [3091-3109] |
| 796 | UGGCCAAGAGCAGGGUUUU | AAAACCCUGCUCUUGGCCA | | [3043-3061] |
| 797 | AGAUCAAAUUCACACUCAA | UUGAGUGUGAAUUUGAUCU | | [2972-2990] |
| 798 | GCAUCUGUGUGACUAAUGG | CCAUUAGUCACACAGAUGC | | [2886-2904] |
| 799 | CGUAAAUACACACACAAAA | UUUUGUGUGUGUAUUUACG | | [2861-2879] |
| 800 | CCGUAAAUACACACACAAA | UUUGUGUGUGUAUUUACGG | | [2860-2878] |
| 801 | CCAAAGUGGUCUUCUCCAG | CUGGAGAAGACCACUUUGG | | [2752-2770] |
| 802 | CAGGAAUCUCACCUUUCAU | AUGAAAGGUGAGAUUCCUG | | [299-317] |
| 803 | AGGAUUGCCUGAAGGGUUC | GAACCCUUCAGGCAAUCCU | | [2609-2627] |
| 804 | GGCUAAGGAUACUAACCAA | UUGGUUAGUAUCCUUAGCC | | [2589-2607] |
| 805 | CUAUUACUGUCAUAGACCA | UGGUCUAUGACAGUAAUAG | | [2538-2556] |
| 806 | UGUUCUUUCUACAGAAGAA | UUCUUCUGUAGAAAGAACA | | [2351-2369] |
| 807 | AGAUGUUCUUUCUACAGAA | UUCUGUAGAAAGAACAUCU | | [2348-2366] |
| 808 | AGAGUGUCUCAACACUUAU | AUAAGUGUUGAGACACUCU | | [2268-2286] |
| 809 | UGCUCAACAAGAGUUCGAA | UUCGAACUCUUGUUGAGCA | | [270-288] |
| 810 | GGUUCAGUAACUUCCCUAA | UUAGGGAAGUUACUGAACC | | [2029-2047] |
| 811 | CUGCCAAAUGCUCAAAUAA | UUAUUUGAGCAUUUGGCAG | | [1764-1782] |
| 812 | GUGCUGCCAAAUGCUCAAA | UUUGAGCAUUUGGCAGCAC | | [1761-1779] |
| 813 | UCUCUUCCAUGAGGAAAUA | UAUUUCCUCAUGGAAGAGA | | [1732-1750] |
| 814 | UGAGUAAACAAAGCAUCUC | GAGAUGCUUUGUUUACUCA | | [1651-1669] |
| 815 | GAAUAGGAGUUUUCCUCUG | CAGAGGAAAACUCCUAUUC | Rb | [1606-1624] |
| 816 | CCCUAAUACCAGAAUAGGA | UCCUAUUCUGGUAUUAGGG | Rb | [1595-1613] |
| 817 | UCAACUGCAUGCUGAUUCU | AGAAUCAGCAUGCAGUUGA | | [199-217] |
| 818 | CCAUUUUUGUCAUUCUGGU | ACCAGAAUGACAAAAAUGG | | [46-64] |
| 819 | GGCCAAUCACUUUGCUGUG | CACAGCAAAGUGAUUGGCC | Rat, Rb, Ms | [1475-1493] |
| 820 | UGCCUGAAUUUCAACUGCA | UGCAGUUGAAAUUCAGGCA | Rat | [189-207] |
| 821 | CAGCCUGCCUGAAUUUCAA | UUGAAAUUCAGGCAGGCUG | Rat, Ms | [184-202] |
| 822 | GCUGUGAUAAGCAGGAGUU | AACUCCUGCUUAUCACAGC | Rat, Rb, Ms | [1123-1141] |
| 823 | UGGCUGUGAUAAGCAGGAG | CUCCUGCUUAUCACAGCCA | Rat, Rb, Ms | [1121-1139] |
| 824 | GUGGCUGUGAUAAGCAGGA | UCCUGCUUAUCACAGCCAC | Rat, Rb, Ms | [1120-1138] |
| 825 | GGGACAAUACAUUUUUGUC | GACAAAAAUGUAUUGUCCC | | [977-995] |
| 826 | GGAAACCCUCCUAUGACUU | AAGUCAUAGGAGGGUUUCC | Rat, Ms | [804-822] |
| 827 | CUGUGCAUAAUAUAACAGU | ACUGUUAUAUUAUGCACAG | | [724-742] |
| 828 | CUGGUAUUACCGGGUUUAU | AUAAACCCGGUAAUACCAG | | [95-113] |
| 829 | UCAUCACGCUGUGCCUCAU | AUGAGGCACAGCGUGAUGA | | [556-574] |
| 830 | CUCGAAAGAGAAUAAAGAA | UUCUUUAUUCUCUUUCGAG | | [481-499] |
| 831 | GACUAUGAAGGUUUUUCUU | AAGAAAAACCUUCAUAGUC | | [4105-4123] |
| 832 | UGAGAAUAUUGGCCCUUUC | GAAAGGGCCAAUAUUCUCA | | [3771-3789] |
| 833 | UGGAGAGGGUACCUCAGUU | AACUGAGGUACCCUCUCCA | | [3742-3760] |
| 834 | GUCAGGCUCUGUUUAUUGU | ACAAUAAACAGAGCCUGAC | | [3696-3714] |
| 835 | CUGCACCUCAGGUAUCAAU | AUUGAUACCUGAGGUGCAG | | [3638-3656] |
| 836 | CUCUGCGAUUCACACCAUU | AAUGGUGUGAAUCGCAGAG | | [347-365] |
| 837 | GAACUCAGUUGUUCUUUGU | ACAAAGAACAACUGAGUUC | | [3475-3493] |
| 838 | GUCUCAUAUUGGUCAUUCC | GGAAUGACCAAUAUGAGAC | | [3293-3311] |
| 839 | CUAAAUCACUGUUAGUGUG | CACACUAACAGUGAUUUAG | | [3026-3044] |
| 840 | UCACACUCAAUAGUAUGUU | AACAUACUAUUGAGUGUGA | | [2981-2999] |
| 841 | CACACAAAAUGGAUCGCAU | AUGCGAUCCAUUUUGUGUG | | [2871-2889] |
| 842 | GGAAUCUGAUUCUCCCUAG | CUAGGGAGAAUCAGAUUCC | | [2559-2577] |
| 843 | CCAAAGGAAUCUGAUUCUC | GAGAAUCAGAUUCCUUUGG | | [2554-2572] |
| 844 | CAACACUUAUUAGUGACAG | CUGUCACUAAUAAGUGUUG | | [2277-2295] |
| 845 | UCAACACUUAUUAGUGACA | UGUCACUAAUAAGUGUUGA | | [2276-2294] |
| 846 | GUGCUGCUCAACAAGAGUU | AACUCUUGUUGAGCAGCAC | | [266-284] |
| 847 | CCCAUCUUACUCCAGGUUU | AAACCUGGAGUAAGAUGGG | | [2074-2092] |
| 848 | UAAUGGUUUUCCCUUAAAG | CUUUAAGGGAAAACCAUUA | | [1841-1859] |
| 849 | CCUAAUACCAGAAUAGGAG | CUCCUAUUCUGGUAUUAGG | Rb | [1596-1614] |
| 850 | UUGCAAGUCAACACCCUAA | UUAGGGUGUUGACUUGCAA | Rb | [1582-1600] |
| 851 | GCCAAUCACUUUGCUGUGC | GCACAGCAAAGUGAUUGGC | Rat, Rb, Ms | [1476-1494] |
| 852 | UCAGGCCAAUCACUUUGCU | AGCAAAGUGAUUGGCCUGA | Rat, Ms | [1472-1490] |
| 853 | GGCUGUGAUAAGCAGGAGU | ACUCCUGCUUAUCACAGCC | Rat, Rb, Ms | [1122-1140] |
| 854 | AGUGCCCAAAGGUGUCCAA | UUGGACACCUUUGGGCACU | | [997-1015] |
| 855 | CCUCCUAUGACUUGGAAAU | AUUUCCAAGUCAUAGGAGG | Rat, Ms | [810-828] |
| 856 | ACCCUCCUAUGACUUGGAA | UUCCAAGUCAUAGGAGGGU | Rat, Ms | [808-826] |
| 857 | UCUGGUAUUACCGGGUUUA | UAAACCCGGUAAUACCAGA | | [94-112] |
| 858 | AUCACGCUGUGCCUCAUAU | AUAUGAGGCACAGCGUGAU | | [558-576] |
| 859 | AUCUCAAUUUUGCUCGAAA | UUUCGAGCAAAAUUGAGAU | | [469-487] |
| 860 | UUCUGAUCCUUAUUCAGUA | UACUGAAUAAGGAUCAGAA | | [413-431] |
| 861 | CUGCUUUUGCAAUUGUGUU | AACACAAUUGCAAAAGCAG | | [4131-4149] |
| 862 | UGAAGGUUUUUCUUAGUUC | GAACUAAGAAAAACCUUCA | | [4110-4128] |
| 863 | CCUGUGUUAUCCAGUUAUG | CAUAACUGGAUAACACAGG | | [3897-3915] |
| 864 | CCAGCUUCAUUUGUUGCUC | GAGCAACAAAUGAAGCUGG | | [3812-3830] |
| 865 | CACAUCUAUUUAAUGUGGA | UCCACAUUAAAUAGAUGUG | | [367-385] |
| 866 | GGGUACCUCAGUUAUAAGG | CCUUAUAACUGAGGUACCC | | [3748-3766] |
| 867 | GAGAGGGUACCUCAGUUAU | AUAACUGAGGUACCCUCUC | | [3744-3762] |
| 868 | CACCUCAGGUAUCAAUUCA | UGAAUUGAUACCUGAGGUG | | [3641-3659] |
| 869 | GCACCUCAGGUAUCAAUUC | GAAUUGAUACCUGAGGUGC | | [3640-3658] |
| 870 | UCACACUGCACCUCAGGUA | UACCUGAGGUGCAGUGUGA | | [3633-3651] |
| 871 | ACUCUGCGAUUCACACCAU | AUGGUGUGAAUCGCAGAGU | | [346-364] |
| 872 | AGAACUCAGUUGUUCUUUG | CAAAGAACAACUGAGUUCU | | [3474-3492] |
| 873 | GGUCACAAAUUUUGAUGCU | AGCAUCAAAAUUUGUGACC | | [3418-3436] |
| 874 | CUGGGAACUAAAACCAGUU | AACUGGUUUUAGUUCCCAG | | [3090-3108] |
| 875 | GGCAUGGAUGAUUGCACUU | AAGUGCAAUCAUCCAUGCC | | [326-344] |
| 876 | GAGAGAGUCUCUAAAUCAC | GUGAUUUAGAGACUCUCUC | | [3016-3034] |
| 877 | CUGAAUAUAUGUUCAAGAG | CUCUUGAACAUAUAUUCAG | | [3000-3018] |
| 878 | ACACUCAAUAGUAUGUUCU | AGAACAUACUAUUGAGUGU | | [2983-3001] |
| 879 | CUCUAUACAGAGAUCAAAU | AUUUGAUCUCUGUAUAGAG | | [2962-2980] |
| 880 | CCUAAAAUUAACAACCCAG | CUGGGUUGUUAAUUUUAGG | | [2933-2951] |
| 881 | GGAUCGCAUCUGUGUGACU | AGUCACACAGAUGCGAUCC | | [2881-2899] |
| 882 | UCCGUAAAUACACACACAA | UUGUGUGUGUAUUUACGGA | | [2859-2877] |
| 883 | GGUGGAGAAUUAACCUCCU | AGGAGGUUAAUUCUCCACC | | [2667-2685] |
| 884 | GCACAUUCUUAUUUGAAGC | GCUUCAAAUAAGAAUGUGC | | [2629-2647] |
| 885 | GACAACUGGACAGGAAUCU | AGAUUCCUGUCCAGUUGUC | | [289-307] |
| 886 | GAAUGAAUUUAGAACCACA | UGUGGUUCUAAAUUCAUUC | | [2460-2478] |
| 887 | GAGAGCGGAUACUCAAGUA | UACUUGAGUAUCCGCUCUC | | [2424-2442] |
| 888 | GGGCCAGAUGUUCUUUCUA | UAGAAAGAACAUCUGGCCC | | [2343-2361] |
| 889 | GUGACAGUAUUGACAUCUG | CAGAUGUCAAUACUGUCAC | | [2289-2307] |
| 890 | GAGUGUCUCAACACUUAUU | AAUAAGUGUUGAGACACUC | | [2269-2287] |
| 891 | CUGCUCAACAAGAGUUCGA | UCGAACUCUUGUUGAGCAG | | [269-287] |
| 892 | GAAACCAAGGAGUAGCUAU | AUAGCUACUCCUUGGUUUC | | [2165-2183] |
| 893 | AGGUUCAGUAACUUCCCUA | UAGGGAAGUUACUGAACCU | | [2028-2046] |
| 894 | CUCUAUGGUUUUGAGAGCA | UGCUCUCAAAACCAUAGAG | | [1906-1924] |
| 895 | CCCUUAAAGGAAUGUCAAA | UUUGACAUUCCUUUAAGGG | | [1851-1869] |
| 896 | GUGGGUUGUGCUGCCAAAU | AUUUGGCAGCACAACCCAC | | [1754-1772] |
| 897 | CUCUUCCAUGAGGAAAUAA | UUAUUUCCUCAUGGAAGAG | | [1733-1751] |
| 898 | GGCCUGAAACAAAAGACUU | AAGUCUUUUGUUUCAGGCC | | [1524-1542] |
| 899 | GCAGCCUGCCUGAAUUUCA | UGAAAUUCAGGCAGGCUGC | Rat, Ms | [183-201] |
| 900 | UCACCAAGGUGGUCACUCA | UGAGUGACCACCUUGGUGA | Rb | [904-922] |
| 901 | GUAUCUCUGUGAGAGGUUG | CAACCUCUCACAGAGAUAC | | [851-869] |
| 902 | CUGUAUCUCUGUGAGAGGU | ACCUCUCACAGAGAUACAG | | [849-867] |
| 903 | UGGAAACCCUCCUAUGACU | AGUCAUAGGAGGGUUUCCA | Ms | [803-821] |
| 904 | CCCAAUCCCUCAGUUUGCU | AGCAAACUGAGGGAUUGGG | | [785-803] |
| 905 | UCAUUGGCCUUGCCAUCCA | UGGAUGGCAAGGCCAAUGA | | [661-679] |
| 906 | AGAGGGUACCUCAGUUAUA | UAUAACUGAGGUACCCUCU | | [3745-3763] |
| 907 | UGCACAUCUAUUUAAUGUG | CACAUUAAAUAGAUGUGCA | | [365-383] |
| 908 | ACCAGGAUGGUUUCUGCUU | AAGCAGAAACCAUCCUGGU | | [3591-3609] |
| 909 | GCGAUUCACACCAUUGCAC | GUGCAAUGGUGUGAAUCGC | | [351-369] |
| 910 | CUAUUCCUUUCGUCUCCCA | UGGGAGACGAAAGGAAUAG | | [3501-3519] |
| 911 | UACACAUACUGAUGUUGUU | AACAACAUCAGUAUGUGUA | | [3365-3383] |
| 912 | GGUAUACACAUACUGAUGU | ACAUCAGUAUGUGUAUACC | | [3361-3379] |
| 913 | GGGCAGAAGACUGGUUUAG | CUAAACCAGUCUUCUGCCC | | [3193-3211] |
| 914 | GAAGGGCAGAAGACUGGUU | AACCAGUCUUCUGCCCUUC | | [3190-3208] |
| 915 | CUCCCAUUUCUGGGAACUA | UAGUUCCCAGAAAUGGGAG | | [3081-3099] |
| 916 | AGAGAGAGUCUCUAAAUCA | UGAUUUAGAGACUCUCUCU | | [3015-3033] |
| 917 | UCUCUAUACAGAGAUCAAA | UUUGAUCUCUGUAUAGAGA | | [2961-2979] |
| 918 | CCCAGAAACAAAAAUCUCU | AGAGAUUUUUGUUUCUGGG | | [2947-2965] |
| 919 | ACAAAAUGGAUCGCAUCUG | CAGAUGCGAUCCAUUUUGU | | [2874-2892] |
| 920 | GACUCUGGCUCAUCUAGUC | GACUAGAUGAGCCAGAGUC | | [2691-2709] |
| 921 | CACAUUCUUAUUUGAAGCA | UGCUUCAAAUAAGAAUGUG | | [2630-2648] |

TABLE A6

CYBB 19-mer oligomer pairs - cytochrome b-245,
beta polypeptide 19 MER

| | | | | |
|---|---|---|---|---|
| | | | | Human-6996020 |
| No. | Sense siRNA | AntiSense siRNA | Other Sp | ORF: 15-1727 |

| | | | | |
|---|---|---|---|---|
| 1 | GGACAAAAAGAAACUAUAA | UUAUAGUUUCUUUUUGUCC | | [1820-1838] |
| 2 | CCACCAAUCUGAAGCUCAA | UUGAGCUUCAGAUUGGUGG | | [1306-1324] |
| 3 | GCUUAAAAAUGGACAAAAA | UUUUUGUCCAUUUUUAAGC | | [1810-1828] |
| 4 | GGACUAGGAUGAACUAAAU | AUUUAGUUCAUCCUAGUCC | | [3454-3472] |
| 5 | CCAGUUAUGAGAUAAAAAA | UUUUUUAUCUCAUAACUGG | | [3907-3925] |
| 6 | GAGUGAGAGAAAAUAAAAA | UUUUUAUUUUCUCUCACUC | | [3246-3264] |
| 7 | GCUCAACUAUUAAAAUGAA | UUCAUUUUAAUAGUUGAGC | | [4239-4257] |
| 8 | GAUGAAGCAUUGUUUUUAA | UUAAAAACAAUGCUUCAUC | | [4080-4098] |
| 9 | GGGUAGGUCUGACAAAUAA | UUAUUUGUCAGACCUACCC | | [3538-3556] |
| 10 | GCACCAUGAUGAGGAGAAA | UUUCUCCUCAUCAUGGUGC | Ms, Rb | [1493-1511] |
| 11 | CCUUGUGCUAUAAAAUAAA | UUUAUUUUAUAGCACAAGG | | [2715-2733] |
| 12 | GGUAUUGAAUCUUUUAAAA | UUUUAAAAGAUUCAAUACC | | [3396-3414] |
| 13 | CAAGACAAUUGCAAGUCAA | UUGACUUGCAAUUGUCUUG | Rb | [1574-1592] |
| 14 | GGUCAUUUGUUUUAAAUAA | UUAUUUAAAACAAAUGACC | | [2824-2842] |
| 15 | AACUGUGCUCAACUAUUAA | UUAAUAGUUGAGCACAGUU | | [4233-4251] |
| 16 | AACCUAUGUGCAUAAUUAA | UUAAUUAUGCACAUAGGUU | | [3791-3809] |
| 17 | AACAGUUUGUGAACAAAAA | UUUUUGUUCACAAACUGUU | | [737-755] |
| 18 | CAAUAAUUCUGAUCCUUAU | AUAAGGAUCAGAAUUAUUG | | [407-425] |
| 19 | GUUUGAUAGUGAUAAGUUA | UAACUUAUCACUAUCAAAC | | [1874-1892] |
| 20 | GAUUAUCUAUGGUAUUGAA | UUCAAUACCAUAGAUAAUC | | [3386-3404] |
| 21 | GGUUGGAGGUGGAGAAUUA | UAAUUCUCCACCUCCAACC | | [2660-2678] |
| 22 | GAAGACAACUGGACAGGAA | UUCCUGUCCAGUUGUCUUC | Rat, Ms | [286-304] |
| 23 | GCUCGAAAGAGAAUAAAGA | UCUUUAUUCUCUUUCGAGC | | [480-498] |
| 24 | AGAUCAUUAGGACAUAAUA | UAUUAUGUCCUAAUGAUCU | | [2242-2260] |
| 25 | GAUUGGAGUUAAUGUAAUA | UAUUACAUUAACUCCAAUC | | [2379-2397] |
| 26 | UGGACAAAAAGAAACUAUA | UAUAGUUUCUUUUUGUCCA | | [1819-1837] |
| 27 | GCAAGUCAACACCCUAAUA | UAUUAGGGUGUUGACUUGC | Rb | [1584-1602] |
| 28 | GCAGAGAGCGGAUACUCAA | UUGAGUAUCCGCUCUCUGC | | [2421-2439] |
| 29 | AGUGCUUGUCAUUAUAAAA | UUUUAUAAUGACAAGCACU | | [3936-3954] |
| 30 | ACUCAAUUUUAGAAUCAAA | UUUGAUUCUAAAAUUGAGU | | [1994-2012] |
| 31 | CCGGAGGUCUUACUUUGAA | UUCAAAGUAAGACCUCCGG | Rb | [605-623] |
| 32 | GACUAAUGGUUUAUUUGUA | UACAAAUAAACCAUUAGUC | | [2896-2914] |
| 33 | UCACAGGCCUGAAACAAAA | UUUUGUUUCAGGCCUGUGA | | [1519-1537] |
| 34 | AGAGUGAGAGAAAAUAAAA | UUUUAUUUUCUCUCACUCU | | [3245-3263] |
| 35 | GAUAAUGAAUUCAAGACAA | UUGUCUUGAAUUCAUUAUC | | [1563-1581] |
| 36 | GACUCAAUUUUAGAAUCAA | UUGAUUCUAAAAUUGAGUC | | [1993-2011] |
| 37 | CAGUUAUGAGAUAAAAAAU | AUUUUUUAUCUCAUAACUG | | [3908-3926] |
| 38 | AAACUGUGCUCAACUAUUA | UAAUAGUUGAGCACAGUUU | | [4232-4250] |
| 39 | GGAACUGGGCUGUGAAUGA | UCAUUCACAGCCCAGUUCC | Rat | [19-37] |
| 40 | GCAUUGUUUUUAAUCUUAA | UUAAGAUUAAAAACAAUGC | | [4086-4104] |
| 41 | GCGAAUAUAGCCUUUCUGA | UCAGAAAGGCUAUAUUCGC | | [3567-3585] |
| 42 | GAAUCUCACCUUUCAUAAA | UUUAUGAAAGGUGAGAUUC | | [302-320] |
| 43 | CAUCCAUGGAGCUGAACGA | UCGUUCAGCUCCAUGGAUG | Rat, Ms, Rb | [674-692] |
| 44 | CAACUUCGGUAAUGAGGAA | UUCCUCAUUACCGAAGUUG | | [3152-3170] |
| 45 | ACCUAUGUGCAUAAUUAAA | UUUAAUUAUGCACAUAGGU | | [3792-3810] |
| 46 | AGUGAGAGAAAAUAAAAAA | UUUUUUAUUUUCUCUCACU | | [3247-3265] |
| 47 | CACCAAUCUGAAGCUCAAA | UUUGAGCUUCAGAUUGGUG | | [1307-1325] |
| 48 | CCUAUGUGCAUAAUUAAAA | UUUUAAUUAUGCACAUAGG | | [3793-3811] |
| 49 | GUUGGAGGUGGAGAAUUAA | UUAAUUCUCCACCUCCAAC | | [2661-2679] |
| 50 | CCUCAAAAUCAGUUUUUAU | AUAAAAACUGAUUUUGAGG | | [2128-2146] |
| 51 | UGGUAUUGAAUCUUUUAAA | UUUAAAAGAUUCAAUACCA | | [3395-3413] |
| 52 | UCCAGUUAUGAGAUAAAAA | UUUUUAUCUCAUAACUGGA | | [3906-3924] |
| 53 | GAGAAUAAAGAACCCUGAA | UUCAGGGUUCUUUAUUCUC | | [488-506] |
| 54 | ACAGUUUGUGAACAAAAAA | UUUUUUGUUCACAAACUGU | | [738-756] |
| 55 | GAGGAAAAGGAAAUAAGGA | UCCUUAUUUCCUUUUCCUC | | [3212-3230] |
| 56 | AAGAGUGCUUGUCAUUAUA | UAUAAUGACAAGCACUCUU | | [3933-3951] |
| 57 | AGUGCGUGCUGCUCAACAA | UUGUUGAGCAGCACGCACU | Rb | [261-279] |
| 58 | AAAAAUGGACAAAAAGAAA | UUUCUUUUUGUCCAUUUUU | | [1814-1832] |
| 59 | CCAUGGAGCUGAACGAAUU | AAUUCGUUCAGCUCCAUGG | Rat, Ms, Rb | [677-695] |
| 60 | UUAUUUGAAGCAUGAAAAA | UUUUUCAUGCUUCAAAUAA | | [2637-2655] |
| 61 | GCAGAAGACUGGUUUAGGA | UCCUAAACCAGUCUUCUGC | | [3195-3213] |
| 62 | CUUAUUUGAAGCAUGAAAA | UUUUCAUGCUUCAAAUAAG | | [2636-2654] |
| 63 | GAUGGUUUCUGCUUAGAGA | UCUCUAAGCAGAAACCAUC | | [3596-3614] |
| 64 | CAGAUUUCUUAAAGUUUGA | UCAAACUUUAAGAAAUCUG | | [3318-3336] |
| 65 | CUUCCAUGAGGAAAUAAAU | AUUUAUUUCCUCAUGGAAG | | [1735-1753] |
| 66 | GUAAGUUUUGUUAAAUGAA | UUCAUUUAACAAAACUUAC | | [2440-2458] |
| 67 | GGUCACUCACCCUUUCAAA | UUUGAAAGGGUGAGUGACC | Rb | [914-932] |
| 68 | GUGUGAAUGCCCGAGUCAA | UUGACUCGGGCAUUCACAC | Rb | [391-409] |
| 69 | UUUGAAGCAUGAAAAAAGA | UCUUUUUUCAUGCUUCAAA | | [2640-2658] |
| 70 | CAAUUUUGCUCGAAAGAGA | UCUCUUUCGAGCAAAAUUG | | [473-491] |
| 71 | UAUGGUCAUUUGUUUUAAA | UUUAAAACAAAUGACCAUA | | [2821-2839] |
| 72 | UCAUUAUUAUCAUACUGAA | UUCAGUAUGAUAAUAAUGA | | [2212-2230] |
| 73 | AGGAAAUAAGGAGAAAAGA | UCUUUUCUCCUUAUUUCCU | | [3219-3237] |
| 74 | CAACUAUUAAAAUGAAUGA | UCAUUCAUUUUAAUAGUUG | | [4242-4260] |
| 75 | UGGGAUAAUGAAUUCAAGA | UCUUGAAUUCAUUAUCCCA | | [1560-1578] |
| 76 | AGGAUGAACUAAAUAAGAA | UUCUUAUUUAGUUCAUCCU | | [3459-3477] |
| 77 | CUCAAAUAAUGCUAAUUGA | UCAAUUAGCAUUAUUUGAG | | [1774-1792] |
| 78 | CCAAGAUAGAAUUAAUUUA | UAAAUUAAUUCUAUCUUGG | | [2484-2502] |
| 79 | GCUGGAAACCCUCCUAUGA | UCAUAGGAGGGUUUCCAGC | Ms | [801-819] |
| 80 | AAGCCUUAAACAAAAUUUA | UAAAUUUUGUUUAAGGCUU | | [2503-2521] |
| 81 | UGUCAAAGAUUGUUUGAUA | UAUCAAACAAUCUUUGACA | | [1863-1881] |
| 82 | CUCAAUUUUAGAAUCAAAA | UUUUGAUUCUAAAAUUGAG | | [1995-2013] |
| 83 | GUUUAGGAGGAAAAGGAAA | UUUCCUUUUCCUCCUAAAC | | [3206-3224] |
| 84 | GAGUUAAUGUAAUACUCAU | AUGAGUAUUACAUUAACUC | | [2384-2402] |
| 85 | GAACUUGGAGACAGGCAAA | UUUGCCUGUCUCCAAGUUC | | [441-459] |
| 86 | GAAUUAAUUUAAAGCCUUA | UAAGGCUUUAAAUUAAUUC | | [2492-2510] |
| 87 | CAUCAUCAUCCUAAAAUUA | UAAUUUUAGGAUGAUGAUG | | [2924-2942] |
| 88 | AUCUGAAGCUCAAAAAGAU | AUCUUUUUGAGCUUCAGAU | | [1312-1330] |
| 89 | GGAAUCUCACCUUUCAUAA | UUAUGAAAGGUGAGAUUCC | | [301-319] |
| 90 | GUUUGAUAUGUAUAGAAUA | UAUUCUAUACAUAUCAAAC | | [3331-3349] |
| 91 | GAAUUGUGACAAAUAUGUA | UACAUAUUUGUCACAAUUC | | [2797-2815] |
| 92 | AAUGGGAGAGUGAGAGAAA | UUUCUCUCACUCUCCCAUU | | [3239-3257] |
| 93 | UAAAAAUGGACAAAAAGAA | UUCUUUUUGUCCAUUUUUA | | [1813-1831] |
| 94 | UAACCUAUGUGCAUAAUUA | UAAUUAUGCACAUAGGUUA | | [3790-3808] |
| 95 | AAAUAAGGAGAAAAGAGAA | UUCUCUUUUCUCCUUAUUU | | [3222-3240] |
| 96 | AAGGGACUAGGAUGAACUA | UAGUUCAUCCUAGUCCCUU | | [3451-3469] |
| 97 | CAACAAGAGUUCGAAGACA | UGUCUUCGAACUCUUGUUG | | [274-292] |
| 98 | AGGAGAAAAGAGAAUGGGA | UCCCAUUCUCUUUUCUCCU | | [3227-3245] |
| 99 | UGUGAAUGCCCGAGUCAAU | AUUGACUCGGGCAUUCACA | | [392-410] |
| 100 | AGAGAAUGGGAGAGUGAGA | UCUCACUCUCCCAUUCUCU | | [3235-3253] |
| 101 | GACAAAAAGAAACUAUAAU | AUUAUAGUUUCUUUUUGUC | | [1821-1839] |
| 102 | CAAAGGUGUCCAAGCUGGA | UCCAGCUUGGACACCUUUG | | [1003-1021] |
| 103 | AAAUGAAAGUUAUCUCAAU | AUUGAGAUAACUUUCAUUU | | [458-476] |
| 104 | UAGGAAUUGUGACAAAUAU | AUAUUUGUCACAAUUCCUA | | [2794-2812] |
| 105 | CCAGGAUGGUUUCUGCUUA | UAAGCAGAAACCAUCCUGG | | [3592-3610] |
| 106 | UGCAUUUCAUUUUCAACAA | UUGUUGAAAAUGAAAUGCA | | [1696-1714] |
| 107 | GAAAAUAAAAAAGGCAAAA | UUUUGCCUUUUUUAUUUUC | | [3254-3272] |
| 108 | AUGAAUGAAUGAAUUUAGA | UCUAAAUUCAUUCAUUCAU | | [2454-2472] |
| 109 | UCAAGUAAGUUUUGUUAAA | UUUAACAAAACUUACUUGA | | [2436-2454] |
| 110 | AGCCUUAAACAAAAUUUAU | AUAAAUUUUGUUUAAGGCU | | [2504-2522] |
| 111 | GAGAAAAGAGAAUGGGAGA | UCUCCCAUUCUCUUUUCUC | | [3229-3247] |
| 112 | AGAAAAUAAAAAAGGCAAA | UUUGCCUUUUUUAUUUUCU | | [3253-3271] |
| 113 | GACAAAUAUGUAUCUGAUA | UAUCAGAUACAUAUUUGUC | | [2804-2822] |
| 114 | GAGAAAAUAAAAAAGGCAA | UUGCCUUUUUUAUUUUCUC | | [3252-3270] |
| 115 | UCCCUUAAAGGAAUGUCAA | UUGACAUUCCUUUAAGGGA | | [1850-1868] |
| 116 | AAACAAAAAUCUCUAUACA | UGUAUAGAGAUUUUUGUUU | | [2952-2970] |
| 117 | AAUUUAAAGCCUUAAACAA | UUGUUUAAGGCUUUAAAUU | | [2497-2515] |
| 118 | UAAAAUAAAUGCAGACUAA | UUAGUCUGCAUUUAUUUUA | | [2725-2743] |
| 119 | GAGUUCGAAGACAACUGGA | UCCAGUUGUCUUCGAACUC | | [280-298] |
| 120 | ACAGGAAUCUCACCUUUCA | UGAAAGGUGAGAUUCCUGU | | [298-316] |
| 121 | AGUCAGUCUGGUACAAAUA | UAUUUGUACCAGACUGACU | Rb | [1276-1294] |
| 122 | GCUAUAAAAUAAAUGCAGA | UCUGCAUUUAUUUUAUAGC | | [2721-2739] |
| 123 | UUAACAACCCAGAAACAAA | UUUGUUUCUGGGUUGUUAA | | [2940-2958] |
| 124 | AUGUAAUACUCAUCAUUUA | UAAAUGAUGAGUAUUACAU | | [2390-2408] |
| 125 | GAAGAAUAUUGGAUUGAUU | AAUCAAUCCAAUAUUCUUC | | [2364-2382] |
| 126 | UGACAGUAUUGACAUCUGA | UCAGAUGUCAAUACUGUCA | | [2290-2308] |
| 127 | UCAUCAUCAUCCUAAAAUU | AAUUUUAGGAUGAUGAUGA | | [2923-2941] |
| 128 | CAUUGCACAUCUAUUUAAU | AUUAAAUAGAUGUGCAAUG | | [362-380] |
| 129 | CAACUGGACAGGAAUCUCA | UGAGAUUCCUGUCCAGUUG | | [291-309] |
| 130 | AGGGCAGAAGACUGGUUUA | UAAACCAGUCUUCUGCCCU | | [3192-3210] |
| 131 | GUGAAUGCCCGAGUCAAUA | UAUUGACUCGGGCAUUCAC | | [393-411] |
| 132 | UCCUUGUGCUAUAAAAUAA | UUAUUUUAUAGCACAAGGA | | [2714-2732] |
| 133 | CUAUGGUAUUGAAUCUUUU | AAAAGAUUCAAUACCAUAG | | [3392-3410] |
| 134 | GAACAAAAAAUCUCAGAAU | AUUCUGAGAUUUUUUGUUC | | [747-765] |
| 135 | CAGUUUGUGAACAAAAAAU | AUUUUUUGUUCACAAACUG | | [739-757] |
| 136 | UGGUCAUUUGUUUUAAAUA | UAUUUAAAACAAAUGACCA | | [2823-2841] |
| 137 | ACAUCUAUUUAAUGUGGAA | UUCCACAUUAAAUAGAUGU | | [368-386] |
| 138 | AAAGGAAAUAAGGAGAAAA | UUUUCUCCUUAUUUCCUUU | | [3217-3235] |
| 139 | UUUAAAGCCUUAAACAAAA | UUUUGUUUAAGGCUUUAAA | | [2499-2517] |
| 140 | AAACCAACUAAGUUAUGAA | UUCAUAACUUAGUUGGUUU | | [3863-3881] |
| 141 | AAGUUUGAUAUGUAUAGAA | UUCUAUACAUAUCAAACUU | | [3329-3347] |
| 142 | GAAAUUUUCCAGAUCAUUA | UAAUGAUCUGGAAAAUUUC | | [2232-2250] |
| 143 | GGUAUUACCGGGUUUAUGA | UCAUAAACCCGGUAAUACC | | [97-115] |
| 144 | CUCUGUUUAUUGUUUCAAU | AUUGAAACAAUAAACAGAG | | [3702-3720] |
| 145 | GUUCGAAGACAACUGGACA | UGUCCAGUUGUCUUCGAAC | | [282-300] |
| 146 | CUUAUUUUCCGUAAAUACA | UGUAUUUACGGAAAAUAAG | | [2852-2870] |
| 147 | GAAAUAAGGAGAAAAGAGA | UCUCUUUUCUCCUUAUUUC | | [3221-3239] |
| 148 | GACAAUACAUUUUUGUCAA | UUGACAAAAAUGUAUUGUC | | [979-997] |
| 149 | CUCAAAAUCAGUUUUUAUA | UAUAAAAACUGAUUUUGAG | | [2129-2147] |
| 150 | CAAAUAAUGCUAAUUGAUA | UAUCAAUUAGCAUUAUUUG | | [1776-1794] |
| 151 | AGUGAUAAGUUACAUUUAU | AUAAAUGUAACUUAUCACU | | [1881-1899] |
| 152 | GAGCUCUAUGGUUUUGAGA | UCUCAAAACCAUAGAGCUC | | [1903-1921] |
| 153 | GAGUAGCUAUAUAUUUCUA | UAGAAAUAUAUAGCUACUC | | [2174-2192] |
| 154 | AAGGCAAAAGGGAGAGAGA | UCUCUCUCCCUUUUGCCUU | | [3264-3282] |
| 155 | CCAUCAUUAUUAUCAUACU | AGUAUGAUAAUAAUGAUGG | | [2209-2227] |
| 156 | CAUCAUCCUAAAAUUAACA | UGUUAAUUUUAGGAUGAUG | | [2927-2945] |
| 157 | UGACAAAUAUGUAUCUGAU | AUCAGAUACAUAUUUGUCA | | [2803-2821] |
| 158 | CCAGAUCUAGCCCAUCUUA | UAAGAUGGGCUAGAUCUGG | | [2064-2082] |
| 159 | GCACUUUUACAAACAUUAU | AUAAUGUUUGUAAAAGUGC | | [1922-1940] |
| 160 | AAAAGGAAAUAAGGAGAAA | UUUCUCCUUAUUUCCUUUU | | [3216-3234] |
| 161 | UGAACAAAAAAUCUCAGAA | UUCUGAGAUUUUUUGUUCA | | [746-764] |
| 162 | AGUCAACACCCUAAUACCA | UGGUAUUAGGGUGUUGACU | Rb | [1587-1605] |
| 163 | UUCAAUUCUUUACACCAAA | UUUGGUGUAAAGAAUUGAA | | [3715-3733] |
| 164 | UGAAUGAAUGAAUUUAGAA | UUCUAAAUUCAUUCAUUCA | | [2455-2473] |
| 165 | AUCCAGUUAUGAGAUAAAA | UUUUAUCUCAUAACUGGAU | | [3905-3923] |
| 166 | CUUAGUAUUUAGCAUUUAU | AUAAAUGCUAAAUACUAAG | | [4042-4060] |
| 167 | CCAAGGAGUAGCUAUAUAU | AUAUAUAGCUACUCCUUGG | | [2169-2187] |
| 168 | GGUACAAAUAUUGCAAUAA | UUAUUGCAAUAUUUGUACC | | [1285-1303] |
| 169 | GGAGGUGGAGAAUUAACCU | AGGUUAAUUCUCCACCUCC | | [2664-2682] |
| 170 | CAAGAUGCGUGGAAACUAC | GUAGUUUCCACGCAUCUUG | | [1143-1161] |
| 171 | UCCUCAAAAUCAGUUUUUA | UAAAAACUGAUUUUGAGGA | | [2127-2145] |
| 172 | UUUAAAAUCUGGUCACAAA | UUUGUGACCAGAUUUUAAA | | [3408-3426] |
| 173 | UAGGAUGAACUAAAUAAGA | UCUUAUUUAGUUCAUCCUA | | [3458-3476] |
| 174 | GUUUGAUACUCUUUCCACA | UGUGGAAAGAGUAUCAAAC | | [2089-2107] |
| 175 | AUCACAGGCCUGAAACAAA | UUUGUUUCAGGCCUGUGAU | | [1518-1536] |
| 176 | AAUUUUGCUCGAAAGAGAA | UUCUCUUUCGAGCAAAAUU | | [474-492] |
| 177 | GUAUGUUCUGAAUAUAUGU | ACAUAUAUUCAGAACAUAC | | [2993-3011] |
| 178 | AAGGUUUUCUAUCUUCAAA | UUUGAAGAUAGAAAACCUU | | [3846-3864] |
| 179 | GCUUGUCAUUAUAAAAGUU | AACUUUUAUAAUGACAAGC | | [3939-3957] |
| 180 | UAUGGUAUUGAAUCUUUUA | UAAAAGAUUCAAUACCAUA | | [3393-3411] |
| 181 | AUGCGUGGAAACUACCUAA | UUAGGUAGUUUCCACGCAU | | [1147-1165] |
| 182 | UCAAAAAGAUCUACUUCUA | UAGAAGUAGAUCUUUUUGA | Rat, Ms, Rb | [1321-1339] |
| 183 | GAAACUGUGACCAGAUCUA | UAGAUCUGGUCACAGUUUC | | [2054-2072] |
| 184 | CAAGAACAGGCUAAGGAUA | UAUCCUUAGCCUGUUCUUG | | [2581-2599] |
| 185 | UUUCCUCUGUGGACCUGAA | UUCAGGUCCACAGAGGAAA | Rb | [1616-1634] |
| 186 | UAUCCAGUUAUGAGAUAAA | UUUAUCUCAUAACUGGAUA | | [3904-3922] |
| 187 | GAAUAUUGAACUUAGGAAU | AUUCCUAAGUUCAAUAUUC | | [2782-2800] |
| 188 | AGCGGAUACUCAAGUAAGU | ACUUACUUGAGUAUCCGCU | | [2427-2445] |
| 189 | CAACUAAGUUAUGAAAGUA | UACUUUCAUAACUUAGUUG | | [3867-3885] |
| 190 | ACUAGGAUGAACUAAAUAA | UUAUUUAGUUCAUCCUAGU | | [3456-3474] |
| 191 | AAGACAACUGGACAGGAAU | AUUCCUGUCCAGUUGUCUU | | [287-305] |
| 192 | AAUUCUUUACACCAAAGUA | UACUUUGGUGUAAAGAAUU | | [3718-3736] |
| 193 | UAACAGUUUGUGAACAAAA | UUUUGUUCACAAACUGUUA | | [736-754] |
| 194 | CAUUGUUUUUAAUCUUAAG | CUUAAGAUUAAAAACAAUG | | [4087-4105] |
| 195 | UUCUUAAAACUGUGCUCAA | UUGAGCACAGUUUUAAGAA | | [4226-4244] |
| 196 | UGGUACAAAUAUUGCAAUA | UAUUGCAAUAUUUGUACCA | | [1284-1302] |
| 197 | UGUGCUCAACUAUUAAAAU | AUUUUAAUAGUUGAGCACA | | [4236-4254] |
| 198 | CUAUGUGCAUAAUUAAAAC | GUUUUAAUUAUGCACAUAG | | [3794-3812] |
| 199 | CAUCACGCUGUGCCUCAUA | UAUGAGGCACAGCGUGAUG | | [557-575] |
| 200 | CUCAGAAUCCUCAAAAUCA | UGAUUUUGAGGAUUCUGAG | | [2120-2138] |
| 201 | UGAUCACAGGCCUGAAACA | UGUUUCAGGCCUGUGAUCA | | [1516-1534] |
| 202 | CUUCCCUAAGAUUAUGAAA | UUUCAUAAUCUUAGGGAAG | | [2039-2057] |
| 203 | CUUAAAGUUUGAUAUGUAU | AUACAUAUCAAACUUUAAG | | [3325-3343] |
| 204 | AUGGAAGUGGGACAAUACA | UGUAUUGUCCCACUUCCAU | | [969-987] |
| 205 | CUUGUGCUAUAAAAUAAAU | AUUUAUUUUAUAGCACAAG | | [2716-2734] |
| 206 | AAUGUUUAGAACUCUUCAA | UUGAAGAGUUCUAAACAUU | | [3136-3154] |
| 207 | GAUUGUUUGAUAGUGAUAA | UUAUCACUAUCAAACAAUC | | [1870-1888] |
| 208 | CUUUGCUGUGCACCAUGAU | AUCAUGGUGCACAGCAAAG | Ms, Rb | [1484-1502] |
| 209 | AAAUGGAAGUGGGACAAUA | UAUUGUCCCACUUCCAUUU | | [967-985] |
| 210 | UCUUAUUUGAAGCAUGAAA | UUUCAUGCUUCAAAUAAGA | | [2635-2653] |
| 211 | UGAAUAUUGAACUUAGGAA | UUCCUAAGUUCAAUAUUCA | | [2781-2799] |
| 212 | UAUCAAUUCAUCUAUUCAA | UUGAAUAGAUGAAUUGAUA | | [3650-3668] |
| 213 | GAGGAAGAAGGAGAAAGAG | CUCUUUCUCCUUCUUCCUC | | [3165-3183] |
| 214 | AGGUUUUCUAUCUUCAAAA | UUUUGAAGAUAGAAAACCU | | [3847-3865] |
| 215 | GAGGGUAGGUCUGACAAAU | AUUUGUCAGACCUACCCUC | | [3536-3554] |
| 216 | CUAAGGAUACUAACCAAUA | UAUUGGUUAGUAUCCUUAG | | [2591-2609] |
| 217 | CAGCUAGCCCUUAUGAAUA | UAUUCAUAAGGGCUAGCUG | | [2768-2786] |
| 218 | AGUUCUUCUGCUUUUGCAA | UUGCAAAAGCAGAAGAACU | | [4124-4142] |
| 219 | ACAAAAAUCUCUAUACAGA | UCUGUAUAGAGAUUUUUGU | | [2954-2972] |
| 220 | CAAUCUGAAGCUCAAAAAG | CUUUUUGAGCUUCAGAUUG | | [1310-1328] |
| 221 | AAAAUUAACAACCCAGAAA | UUUCUGGGUUGUUAAUUUU | | [2936-2954] |
| 222 | UAGUGAUAAGUUACAUUUA | UAAAUGUAACUUAUCACUA | | [1880-1898] |
| 223 | CUCCCUAGGGUCAAGAACA | UGUUCUUGACCCUAGGGAG | | [2570-2588] |
| 224 | GGAAGUGGGACAAUACAUU | AAUGUAUUGUCCCACUUCC | | [971-989] |
| 225 | AUGUUGUUUUGAUUAUCUA | UAGAUAAUCAAAACAACAU | | [3376-3394] |
| 226 | CCCUCCUAUGACUUGGAAA | UUUCCAAGUCAUAGGAGGG | Rat, Ms | [809-827] |
| 227 | GGUGGUGAUGUUAGUGGGA | UCCCACUAACAUCACCACC | Rat, Ms, Rb | [1220-1238] |
| 228 | UUAUCAUACUGAAGGAAAU | AUUUCCUUCAGUAUGAUAA | | [2218-2236] |
| 229 | UCUUGGACUCAAUUUUAGA | UCUAAAAUUGAGUCCAAGA | | [1988-2006] |
| 230 | CAUCCAUUCUCAAGUCAGU | ACUGACUUGAGAAUGGAUG | | [1264-1282] |
| 231 | AUUUAAAGCCUUAAACAAA | UUUGUUUAAGGCUUUAAAU | | [2498-2516] |
| 232 | CAAUAGUAUGUUCUGAAUA | UAUUCAGAACAUACUAUUG | | [2988-3006] |
| 233 | UCUGGUACAAAUAUUGCAA | UUGCAAUAUUUGUACCAGA | | [1282-1300] |
| 234 | CAAAAUGGAUCGCAUCUGU | ACAGAUGCGAUCCAUUUUG | | [2875-2893] |
| 235 | AGAGAAUAAAGAACCCUGA | UCAGGGUUCUUUAUUCUCU | | [487-505] |
| 236 | UUAUUAACAGGUACUCUAA | UUAGAGUACCUGUUAAUAA | | [4057-4075] |
| 237 | CUCAACUAUUAAAAUGAAU | AUUCAUUUUAAUAGUUGAG | | [4240-4258] |
| 238 | CUCCGAGAGUGUUUCUCCA | UGGAGAAACACUCUCGGAG | | [3828-3846] |
| 239 | GGUUUUCCCUUAAAGGAAU | AUUCCUUUAAGGGAAAACC | | [1845-1863] |
| 240 | GUUUGUGAACAAAAAAUCU | AGAUUUUUUGUUCACAAAC | | [741-759] |
| 241 | CAUUUUUUUCCUCUCAGUA | UACUGAGAGGAAAAAAAUG | | [1943-1961] |
| 242 | UGGUUUUCCCUUAAAGGAA | UUCCUUUAAGGGAAAACCA | | [1844-1862] |
| 243 | GAUUGAUUGGAGUUAAUGU | ACAUUAACUCCAAUCAAUC | | [2375-2393] |
| 244 | UGUAUUAUAUCAUCAUCAU | AUGAUGAUGAUAUAAUACA | | [2911-2929] |
| 245 | UGUGCCUCAUAUUAAUUAU | AUAAUUAAUAUGAGGCACA | | [565-583] |
| 246 | GAGUCAGGCUCUGUUUAUU | AAUAAACAGAGCCUGACUC | | [3694-3712] |
| 247 | AAAAUAAAGGAAUGCCCAA | UUGGGCAUUCCUUUAUUUU | | [771-789] |
| 248 | AGACAAUUGCAAGUCAACA | UGUUGACUUGCAAUUGUCU | Rb | [1576-1594] |
| 249 | CCUUAUGAAUAUUGAACUU | AAGUUCAAUAUUCAUAAGG | | [2776-2794] |
| 250 | UAAAAUUAACAACCCAGAA | UUCUGGGUUGUUAAUUUUA | | [2935-2953] |
| 251 | AAUUAACAACCCAGAAACA | UGUUUCUGGGUUGUUAAUU | | [2938-2956] |
| 252 | UUUCUAUCUUCAAAACCAA | UUGGUUUUGAAGAUAGAAA | | [3851-3869] |
| 253 | AACCAACUAAGUUAUGAAA | UUUCAUAACUUAGUUGGUU | | [3864-3882] |
| 254 | UCUUAAAGUUUGAUAUGUA | UACAUAUCAAACUUUAAGA | | [3324-3342] |
| 255 | UGCUGUGCACCAUGAUGAG | CUCAUCAUGGUGCACAGCA | Ms, Rb | [1487-1505] |
| 256 | AGUAAGUUUUGUUAAAUGA | UCAUUUAACAAAACUUACU | | [2439-2457] |
| 257 | CAUACUCCAGUUUACUAAU | AUUAGUAAACUGGAGUAUG | | [2310-2328] |
| 258 | UUGAGAGCACUUUUACAAA | UUUGUAAAAGUGCUCUCAA | | [1916-1934] |
| 259 | GAUCACAGGCCUGAAACAA | UUGUUUCAGGCCUGUGAUC | | [1517-1535] |
| 260 | GUUCAAAAUGGAAGUGGGA | UCCCACUUCCAUUUUGAAC | | [962-980] |
| 261 | UUUUCUUUUUGUUCUUAGA | UCUAAGAACAAAAAGAAAA | | [3058-3076] |
| 262 | UCAUUUUCAACAAGGAAAA | UUUUCCUUGUUGAAAAUGA | Ms, Rb | [1702-1720] |
| 263 | CAGAAACAAAAAUCUCUAU | AUAGAGAUUUUUGUUUCUG | | [2949-2967] |
| 264 | CAAGUAAGUUUUGUUAAAU | AUUUAACAAAACUUACUUG | | [2437-2455] |
| 265 | GUUAAAUGAAUGAAUGAAU | AUUCAUUCAUUCAUUUAAC | | [2449-2467] |
| 266 | GAAGGGUUCUGCACAUUCU | AGAAUGUGCAGAACCCUUC | | [2619-2637] |
| 267 | AAACCAAGGAGUAGCUAUA | UAUAGCUACUCCUUGGUUU | | [2166-2184] |
| 268 | GAACUAAAACCAGUUUUAU | AUAAAACUGGUUUUAGUUC | | [3094-3112] |
| 269 | AAAGCAUCUCCAACUCUGA | UCAGAGUUGGAGAUGCUUU | | [1660-1678] |
| 270 | CACCAAAGUAUGAACUGGA | UCCAGUUCAUACUUUGGUG | | [3727-3745] |
| 271 | AACAACCCAGAAACAAAAA | UUUUUGUUUCUGGGUUGUU | | [2942-2960] |
| 272 | GAGUGUUUCUCCAAGGUUU | AAACCUUGGAGAAACACUC | | [3834-3852] |
| 273 | AAGAUUGUUUGAUAGUGAU | AUCACUAUCAAACAAUCUU | | [1868-1886] |
| 274 | AUAGUAUGUUCUGAAUAUA | UAUAUUCAGAACAUACUAU | | [2990-3008] |
| 275 | CUUCCUCCACCAAAACCAU | AUGGUUUUGGUGGAGGAAG | | [586-604] |
| 276 | AAUAGUAUGUUCUGAAUAU | AUAUUCAGAACAUACUAUU | | [2989-3007] |
| 277 | GUGCUAUAAAAUAAAUGCA | UGCAUUUAUUUUAUAGCAC | | [2719-2737] |
| 278 | UAGCAUUUAUUAACAGGUA | UACCUGUUAAUAAAUGCUA | | [4051-4069] |
| 279 | CUGAUGUUGUUUUGAUUAU | AUAAUCAAAACAACAUCAG | | [3373-3391] |
| 280 | AAAUGUUUAGAACUCUUCA | UGAAGAGUUCUAAACAUUU | | [3135-3153] |
| 281 | UAGGGAAAAGAUUCUUGGA | UCCAAGAAUCUUUUCCCUA | | [1976-1994] |
| 282 | CAAAAUGAAAGUUAUCUCA | UGAGAUAACUUUCAUUUUG | | [456-474] |
| 283 | AGUUUGAUAUGUAUAGAAU | AUUCUAUACAUAUCAAACU | | [3330-3348] |
| 284 | UCAUCAUCCUAAAAUUAAC | GUUAAUUUUAGGAUGAUGA | | [2926-2944] |
| 285 | UUAGGAAUUGUGACAAAUA | UAUUUGUCACAAUUCCUAA | | [2793-2811] |
| 286 | AAAGAUUGUUUGAUAGUGA | UCACUAUCAAACAAUCUUU | | [1867-1885] |
| 287 | AUUCUUAUUUGAAGCAUGA | UCAUGCUUCAAAUAAGAAU | | [2633-2651] |
| 288 | AAUCUCACCUUUCAUAAAA | UUUUAUGAAAGGUGAGAUU | | [303-321] |
| 289 | AGGUAUCAAUUCAUCUAUU | AAUAGAUGAAUUGAUACCU | | [3647-3665] |
| 290 | CACUGCACCUCAGGUAUCA | UGAUACCUGAGGUGCAGUG | | [3636-3654] |
| 291 | AUAGCCUUUCUGAAAUGUA | UACAUUUCAGAAAGGCUAU | | [3573-3591] |
| 292 | AGUUAUAAGGAGUCUGAGA | UCUCAGACUCCUUAUAACU | | [3757-3775] |
| 293 | AGCUCAAAAAGAUCUACUU | AAGUAGAUCUUUUUGAGCU | | [1318-1336] |
| 294 | UGGUCACUCACCCUUUCAA | UUGAAAGGGUGAGUGACCA | Rb | [913-931] |
| 295 | AAUCUGAAGCUCAAAAAGA | UCUUUUUGAGCUUCAGAUU | | [1311-1329] |
| 296 | UGUUAAAUGAAUGAAUGAA | UUCAUUCAUUCAUUUAACA | | [2448-2466] |
| 297 | UGAAUUCAAGACAAUUGCA | UGCAAUUGUCUUGAAUUCA | | [1568-1586] |
| 298 | CACAAAUUUUGAUGCUGAG | CUCAGCAUCAAAAUUUGUG | | [3421-3439] |
| 299 | UAGCCCUUAUGAAUAUUGA | UCAAUAUUCAUAAGGGCUA | | [2772-2790] |
| 300 | GUUUAUUGUUUCAAUUCUU | AAGAAUUGAAACAAUAAAC | | [3706-3724] |
| 301 | UAGUAUGUUCUGAAUAUAU | AUAUAUUCAGAACAUACUA | | [2991-3009] |
| 302 | ACUAAGUUAUGAAAGUAGA | UCUACUUUCAUAACUUAGU | | [3869-3887] |
| 303 | GUCUGGUACAAAUAUUGCA | UGCAAUAUUUGUACCAGAC | | [1281-1299] |
| 304 | CUUCUAUUACUGUCAUAGA | UCUAUGACAGUAAUAGAAG | | [2535-2553] |
| 305 | CGUGCUGCUCAACAAGAGU | ACUCUUGUUGAGCAGCACG | | [265-283] |
| 306 | GAUGCGUGGAAACUACCUA | UAGGUAGUUUCCACGCAUC | | [1146-1164] |
| 307 | UCAAGACAAUUGCAAGUCA | UGACUUGCAAUUGUCUUGA | Rb | [1573-1591] |
| 308 | GUGUUUGUGAAAUUUGAAU | AUUCAAAUUUCACAAACAC | | [4145-4163] |
| 309 | UAAGGAGUCUGAGAAUAUU | AAUAUUCUCAGACUCCUUA | | [3762-3780] |
| 310 | AUUUCAUUUUCAACAAGGA | UCCUUGUUGAAAAUGAAAU | | [1699-1717] |
| 311 | GGACAGGAAUCUCACCUUU | AAAGGUGAGAUUCCUGUCC | | [296-314] |
| 312 | CAUCUUACUCCAGGUUUGA | UCAAACCUGGAGUAAGAUG | | [2076-2094] |
| 313 | AAAGAGAAUGGGAGAGUGA | UCACUCUCCCAUUCUCUUU | | [3233-3251] |
| 314 | GAUCAAAUUCACACUCAAU | AUUGAGUGUGAAUUUGAUC | | [2973-2991] |
| 315 | UAAGAGUGCUUGUCAUUAU | AUAAUGACAAGCACUCUUA | | [3932-3950] |
| 316 | UCAAUAGUAUGUUCUGAAU | AUUCAGAACAUACUAUUGA | | [2987-3005] |
| 317 | AAAGUUUGAUAUGUAUAGA | UCUAUACAUAUCAAACUUU | | [3328-3346] |
| 318 | GUUCUGAAUAUAUGUUCAA | UUGAACAUAUAUUCAGAAC | | [2997-3015] |
| 319 | GUUAUGAGAUAAAAAAUGA | UCAUUUUUUAUCUCAUAAC | | [3910-3928] |
| 320 | UGAAGCUCAAAAAGAUCUA | UAGAUCUUUUUGAGCUUCA | | [1315-1333] |
| 321 | AGAGAAAAUAAAAAAGGCA | UGCCUUUUUUAUUUUCUCU | | [3251-3269] |
| 322 | UGUUGUUUUGAUUAUCUAU | AUAGAUAAUCAAAACAACA | | [3377-3395] |
| 323 | CAAAAAGAAACUAUAAUGU | ACAUUAUAGUUUCUUUUUG | | [1823-1841] |
| 324 | UAGCCUUUCUGAAAUGUAC | GUACAUUUCAGAAAGGCUA | | [3574-3592] |
| 325 | ACUGCACCUCAGGUAUCAA | UUGAUACCUGAGGUGCAGU | | [3637-3655] |
| 326 | AGAAGGAAACCAAGGAGUA | UACUCCUUGGUUUCCUUCU | | [2160-2178] |
| 327 | CAGAAUCCUCAAAAUCAGU | ACUGAUUUUGAGGAUUCUG | | [2122-2140] |
| 328 | CAUCUAUUCAACAGAUAUU | AAUAUCUGUUGAAUAGAUG | | [3658-3676] |
| 329 | AUGGUAUUGAAUCUUUUAA | UUAAAAGAUUCAAUACCAU | | [3394-3412] |
| 330 | AUAACAGUUUGUGAACAAA | UUUGUUCACAAACUGUUAU | | [735-753] |
| 331 | GUAUCAAUUCAUCUAUUCA | UGAAUAGAUGAAUUGAUAC | | [3649-3667] |
| 332 | UUUCAAUUCUUUACACCAA | UUGGUGUAAAGAAUUGAAA | | [3714-3732] |
| 333 | UGAUUGGAGUUAAUGUAAU | AUUACAUUAACUCCAAUCA | | [2378-2396] |
| 334 | AAACCAUCCGGAGGUCUUA | UAAGACCUCCGGAUGGUUU | | [598-616] |
| 335 | CAGGAUGGUUUCUGCUUAG | CUAAGCAGAAACCAUCCUG | | [3593-3611] |
| 336 | UACUGAUGUUGUUUUGAUU | AAUCAAAACAACAUCAGUA | | [3371-3389] |
| 337 | AGUCCUGCUCCUUGUGCUA | UAGCACAAGGAGCAGGACU | | [2706-2724] |
| 338 | ACUAAUGGUUUAUUUGUAU | AUACAAAUAAACCAUUAGU | | [2897-2915] |
| 339 | AAUUCACACUCAAUAGUAU | AUACUAUUGAGUGUGAAUU | | [2978-2996] |
| 340 | CAGAUGCAGGAAAGGAACA | UGUUCCUUUCCUGCAUCUG | Ms | [1404-1422] |
| 341 | GCUAUGAGGUGGUGAUGUU | AACAUCACCACCUCAUAGC | Rat, Ms, Rb | [1213-1231] |
| 342 | UGCUCAACUAUUAAAAUGA | UCAUUUUAAUAGUUGAGCA | | [4238-4256] |
| 343 | UUGUGAACAAAAAAUCUCA | UGAGAUUUUUUGUUCACAA | | [743-761] |
| 344 | UAAUAUAACAGUUUGUGAA | UUCACAAACUGUUAUAUUA | | [731-749] |
| 345 | AAUAUAUGUUCAAGAGAGA | UCUCUCUUGAACAUAUAUU | | [3003-3021] |
| 346 | CAGUAUUGACAUCUGAGCA | UGCUCAGAUGUCAAUACUG | | [2293-2311] |
| 347 | CUAUAUAUUUCUACUUUGU | ACAAAGUAGAAAUAUAUAG | | [2180-2198] |
| 348 | AUAUGGUCAUUUGUUUUAA | UUAAAACAAAUGACCAUAU | | [2820-2838] |
| 349 | GAAGCAUUGUUUUUAAUCU | AGAUUAAAAACAAUGCUUC | | [4083-4101] |
| 350 | CUAUUCAACAGAUAUUUAU | AUAAAUAUCUGUUGAAUAG | | [3661-3679] |
| 351 | UAUUUGAAGCAUGAAAAAA | UUUUUUCAUGCUUCAAAUA | | [2638-2656] |
| 352 | GGUACCUCAGUUAUAAGGA | UCCUUAUAACUGAGGUACC | | [3749-3767] |
| 353 | UAAUUUAAAGCCUUAAACA | UGUUUAAGGCUUUAAAUUA | | [2496-2514] |
| 354 | AGCACUUUUACAAACAUUA | UAAUGUUUGUAAAAGUGCU | | [1921-1939] |
| 355 | CACACUGCACCUCAGGUAU | AUACCUGAGGUGCAGUGUG | | [3634-3652] |
| 356 | GUAGCUAUAUAUUUCUACU | AGUAGAAAUAUAUAGCUAC | | [2176-2194] |
| 357 | CAUUUAUGUGGAGCUCUAU | AUAGAGCUCCACAUAAAUG | | [1893-1911] |
| 358 | UUGUUAAAUGAAUGAAUGA | UCAUUCAUUCAUUUAACAA | | [2447-2465] |
| 359 | GAUAGUGGGUCCCAUGUUU | AAACAUGGGACCCACUAUC | | [830-848] |
| 360 | AGGCUAAGGAUACUAACCA | UGGUUAGUAUCCUUAGCCU | | [2588-2606] |
| 361 | AGAGCGGAUACUCAAGUAA | UUACUUGAGUAUCCGCUCU | | [2425-2443] |
| 362 | AGAAGAAUAUUGGAUUGAU | AUCAAUCCAAUAUUCUUCU | | [2363-2381] |
| 363 | GCUUUGUAUGUGAAUAAUU | AAUUAUUCACAUACAAAGC | | [4172-4190] |
| 364 | AGAUCUAGCCCAUCUUACU | AGUAAGAUGGGCUAGAUCU | | [2066-2084] |
| 365 | AGGUGGUCAUCACCAAGGU | ACCUUGGUGAUGACCACCU | Ms, Rb | [895-913] |
| 366 | UAAUUCUGAUCCUUAUUCA | UGAAUAAGGAUCAGAAUUA | | [410-428] |
| 367 | UGUGACUAAUGGUUUAUUU | AAAUAAACCAUUAGUCACA | | [2893-2911] |
| 368 | CAGUAAUGUCAGUGGAAGU | ACUUCCACUGACAUUACUG | | [1957-1975] |
| 369 | GGUUUUCUUUUUGUUCUUA | UAAGAACAAAAAGAAAACC | | [3056-3074] |
| 370 | CUGAUAUGGUCAUUUGUUU | AAACAAAUGACCAUAUCAG | | [2817-2835] |
| 371 | GACAUCUGAGCAUACUCCA | UGGAGUAUGCUCAGAUGUC | | [2300-2318] |
| 372 | AUUGCAAGUCAACACCCUA | UAGGGUGUUGACUUGCAAU | Rb | [1581-1599] |
| 373 | UGUUGAGAGUGUCUCAACA | UGUUGAGACACUCUCAACA | | [2263-2281] |
| 374 | UCUUCCAUGAGGAAAUAAA | UUUAUUUCCUCAUGGAAGA | | [1734-1752] |
| 375 | GGAAAUAAAUGUGGGUUGU | ACAACCCACAUUUAUUUCC | | [1744-1762] |
| 376 | AGAUUGUUUGAUAGUGAUA | UAUCACUAUCAAACAAUCU | | [1869-1887] |
| 377 | CCCAAAGUGGUCUUCUCCA | UGGAGAAGACCACUUUGGG | | [2751-2769] |
| 378 | UCUUUACACCAAAGUAUGA | UCAUACUUUGGUGUAAAGA | | [3721-3739] |
| 379 | GCAUAAUAUAACAGUUUGU | ACAAACUGUUAUAUUAUGC | | [728-746] |
| 380 | UGUGCAUAAUAUAACAGUU | AACUGUUAUAUUAUGCACA | | [725-743] |
| 381 | ACUUUGCUGUGCACCAUGA | UCAUGGUGCACAGCAAAGU | Ms, Rb | [1483-1501] |
| 382 | GUUUGCAGAUCUGCUGCAA | UUGCAGCAGAUCUGCAAAC | | [1373-1391] |
| 383 | UCUACAGAAGAAUAUUGGA | UCCAAUAUUCUUCUGUAGA | | [2358-2376] |
| 384 | AAAAUGAAAGUUAUCUCAA | UUGAGAUAACUUUCAUUUU | | [457-475] |
| 385 | UCCGGAGGUCUUACUUUGA | UCAAAGUAAGACCUCCGGA | Rb | [604-622] |
| 386 | GAAACAAAAGACUUUGUAU | AUACAAAGUCUUUUGUUUC | | [1529-1547] |
| 387 | GUAUGUGAAUAAUUCUAGC | GCUAGAAUUAUUCACAUAC | | [4177-4195] |
| 388 | UUAAAAAUGGACAAAAAGA | UCUUUUUGUCCAUUUUUAA | | [1812-1830] |
| 389 | AAUGGACAAAAAGAAACUA | UAGUUUCUUUUUGUCCAUU | | [1817-1835] |
| 390 | GGUAGGUCUGACAAAUAAG | CUUAUUUGUCAGACCUACC | | [3539-3557] |
| 391 | GUACAAAUAUUGCAAUAAC | GUUAUUGCAAUAUUUGUAC | | [1286-1304] |
| 392 | UGCUUGUCAUUAUAAAAGU | ACUUUUAUAAUGACAAGCA | | [3938-3956] |
| 393 | CACUUAUUAGUGACAGUAU | AUACUGUCACUAAUAAGUG | | [2280-2298] |
| 394 | UACCUAAGAUAGCGGUUGA | UCAACCGCUAUCUUAGGUA | | [1159-1177] |
| 395 | CUAUUAAAAUGAAUGAGCU | AGCUCAUUCAUUUUAAUAG | | [4245-4263] |
| 396 | AUCUGGUCACAAAUUUUGA | UCAAAAUUUGUGACCAGAU | | [3414-3432] |
| 397 | AGAAUGGGAGAGUGAGAGA | UCUCUCACUCUCCCAUUCU | | [3237-3255] |
| 398 | UUUCAUUUUCAACAAGGAA | UUCCUUGUUGAAAAUGAAA | | [1700-1718] |
| 399 | UUUUGAGAGCACUUUUACA | UGUAAAAGUGCUCUCAAAA | | [1914-1932] |
| 400 | AAAACUGUGCUCAACUAUU | AAUAGUUGAGCACAGUUUU | | [4231-4249] |
| 401 | ACAAGAGUUCGAAGACAAC | GUUGUCUUCGAACUCUUGU | | [276-294] |
| 402 | AUGUGAUCACAGGCCUGAA | UUCAGGCCUGUGAUCACAU | | [1513-1531] |
| 403 | ACAUGUUGAGAGUGUCUCA | UGAGACACUCUCAACAUGU | | [2260-2278] |
| 404 | CUUAAAAAUGGACAAAAAG | CUUUUUGUCCAUUUUUAAG | | [1811-1829] |
| 405 | AGCAUUGUUUUUAAUCUUA | UAAGAUUAAAAACAAUGCU | | [4085-4103] |
| 406 | GUGAGAGAAAAUAAAAAAG | CUUUUUUAUUUUCUCUCAC | | [3248-3266] |
| 407 | CUGAGGGUAGGUCUGACAA | UUGUCAGACCUACCCUCAG | | [3534-3552] |
| 408 | GAACUUAGGAAUUGUGACA | UGUCACAAUUCCUAAGUUC | | [2789-2807] |
| 409 | GUUCUUUGUCAUACUACUA | UAGUAGUAUGACAAAGAAC | | [3485-3503] |
| 410 | AAUGAGGAAGAAGGAGAAA | UUUCUCCUUCUUCCUCAUU | | [3162-3180] |
| 411 | UGGUCAUCACCAAGGUGGU | ACCACCUUGGUGAUGACCA | Ms, Rb | [898-916] |
| 412 | UGUUCUGAAUAUAUGUUCA | UGAACAUAUAUUCAGAACA | | [2996-3014] |
| 413 | AAGGGUUCUGCACAUUCUU | AAGAAUGUGCAGAACCCUU | | [2620-2638] |
| 414 | UUGGAGACAGGCAAAAUGA | UCAUUUUGCCUGUCUCCAA | | [445-463] |
| 415 | CCAGAAUAGGAGUUUUCCU | AGGAAAACUCCUAUUCUGG | Rb | [1603-1621] |
| 416 | AUCUCUAUACAGAGAUCAA | UUGAUCUCUGUAUAGAGAU | | [2960-2978] |
| 417 | UGUUCUUUGUCAUACUACU | AGUAGUAUGACAAAGAACA | | [3484-3502] |
| 418 | AAUUGUGACAAAUAUGUAU | AUACAUAUUUGUCACAAUU | | [2798-2816] |
| 419 | AUUCUUGGACUCAAUUUUA | UAAAAUUGAGUCCAAGAAU | | [1986-2004] |
| 420 | AAUAUAGCCUUUCUGAAAU | AUUUCAGAAAGGCUAUAUU | | [3570-3588] |
| 421 | UAAGAUUAUGAAACUGUGA | UCACAGUUUCAUAAUCUUA | | [2045-2063] |
| 422 | GAGAGAAAAUAAAAAAGGC | GCCUUUUUUAUUUUCUCUC | | [3250-3268] |
| 423 | UACUGAAGGAAAUUUUCCA | UGGAAAAUUUCCUUCAGUA | | [2224-2242] |
| 424 | AUCAUCAUCCUAAAAUUAA | UUAAUUUUAGGAUGAUGAU | | [2925-2943] |
| 425 | UUGUAUGUGAAUAAUUCUA | UAGAAUUAUUCACAUACAA | | [4175-4193] |
| 426 | AGUUUUGUUAAAUGAAUGA | UCAUUCAUUUAACAAAACU | | [2443-2461] |
| 427 | GCUUCCUCAGCUACAACAU | AUGUUGUAGCUGAGGAAGC | Rb | [1429-1447] |
| 428 | AAGGAAUGUCAAAGAUUGU | ACAAUCUUUGACAUUCCUU | | [1857-1875] |
| 429 | GAUAGUGAUAAGUUACAUU | AAUGUAACUUAUCACUAUC | | [1878-1896] |
| 430 | UGAUAGUGAUAAGUUACAU | AUGUAACUUAUCACUAUCA | | [1877-1895] |
| 431 | UCAUUAGGACAUAAUACAU | AUGUAUUAUGUCCUAAUGA | | [2245-2263] |
| 432 | GAAACUAUAAUGUAAUGGU | ACCAUUACAUUAUAGUUUC | | [1829-1847] |
| 433 | UGCUUAAAAAUGGACAAAA | UUUUGUCCAUUUUUAAGCA | | [1809-1827] |
| 434 | AAGUGGUCUUCUCCAGCUA | UAGCUGGAGAAGACCACUU | | [2755-2773] |
| 435 | AAGCAUUGUUUUUAAUCUU | AAGAUUAAAAACAAUGCUU | | [4084-4102] |
| 436 | ACUUAUUAGUGACAGUAUU | AAUACUGUCACUAAUAAGU | | [2281-2299] |
| 437 | GAAGAAGGAGAAAGAGCUG | CAGCUCUUUCUCCUUCUUC | | [3168-3186] |
| 438 | CCUUUGAGUGGUUUGCAGA | UCUGCAAACCACUCAAAGG | Ms, Rb | [1363-1381] |
| 439 | GGCUUCCUCAGCUACAACA | UGUUGUAGCUGAGGAAGCC | Rb | [1428-1446] |
| 440 | CAUGGAGCUGAACGAAUUG | CAAUUCGUUCAGCUCCAUG | Rat, Ms, Rb | [678-696] |
| 441 | CUUUGAGUGGUUUGCAGAU | AUCUGCAAACCACUCAAAG | | [1364-1382] |
| 442 | CCAAAGUAUGAACUGGAGA | UCUCCAGUUCAUACUUUGG | | [3729-3747] |
| 443 | UAUAACAGUUUGUGAACAA | UUGUUCACAAACUGUUAUA | | [734-752] |
| 444 | CAUAAUACAUGUUGAGAGU | ACUCUCAACAUGUAUUAUG | | [2254-2272] |
| 445 | AUAAUGAAUUCAAGACAAU | AUUGUCUUGAAUUCAUUAU | | [1564-1582] |
| 446 | AUCCAUGGAGCUGAACGAA | UUCGUUCAGCUCCAUGGAU | Rat, Ms, Rb | [675-693] |
| 447 | UGCCUCAUAUUAAUUAUCA | UGAUAAUUAAUAUGAGGCA | | [567-585] |
| 448 | UAGGGUCAAGAACAGGCUA | UAGCCUGUUCUUGACCCUA | | [2575-2593] |
| 449 | CACUUAGUAUUUAGCAUUU | AAAUGCUAAAUACUAAGUG | | [4040-4058] |
| 450 | AUUAUAUCAUCAUCAUCAU | AUGAUGAUGAUGAUAUAAU | | [2914-2932] |
| 451 | CUCAAGUAAGUUUUGUUAA | UUAACAAAACUUACUUGAG | | [2435-2453] |
| 452 | AUUUUUUUCCUCUCAGUAA | UUACUGAGAGGAAAAAAAU | | [1944-1962] |
| 453 | CUCAAUUUUGCUCGAAAGA | UCUUUCGAGCAAAAUUGAG | | [471-489] |
| 454 | GAUGUUGUUUUGAUUAUCU | AGAUAAUCAAAACAACAUC | | [3375-3393] |
| 455 | AUCUCACCUUUCAUAAAAU | AUUUUAUGAAAGGUGAGAU | | [304-322] |
| 456 | AUUGAUUGGAGUUAAUGUA | UACAUUAACUCCAAUCAAU | | [2376-2394] |
| 457 | CAUGUUGAGAGUGUCUCAA | UUGAGACACUCUCAACAUG | | [2261-2279] |
| 458 | GGUUCAAAAUGGAAGUGGG | CCCACUUCCAUUUUGAACC | | [961-979] |
| 459 | AAAGAAGAAGGAAACCAAG | CUUGGUUUCCUUCUUCUUU | | [2155-2173] |
| 460 | AGAAAUAACUUCUAUUACU | AGUAAUAGAAGUUAUUUCU | | [2527-2545] |
| 461 | UGAGUCAGGCUCUGUUUAU | AUAAACAGAGCCUGACUCA | | [3693-3711] |
| 462 | UUGAUUGGAGUUAAUGUAA | UUACAUUAACUCCAAUCAA | | [2377-2395] |
| 463 | UUAUUAUCAUACUGAAGGA | UCCUUCAGUAUGAUAAUAA | | [2215-2233] |
| 464 | AGGAUACUAACCAAUAGGA | UCCUAUUGGUUAGUAUCCU | | [2594-2612] |
| 465 | UGUUCACACUGCACCUCAG | CUGAGGUGCAGUGUGAACA | | [3630-3648] |
| 466 | AUGAAGCAUUGUUUUUAAU | AUUAAAAACAAUGCUUCAU | | [4081-4099] |
| 467 | GAAUCUUUUAAAAUCUGGU | ACCAGAUUUUAAAAGAUUC | | [3402-3420] |
| 468 | UGAUGAAGCAUUGUUUUUA | UAAAAACAAUGCUUCAUCA | | [4079-4097] |
| 469 | AAUAAAGAACCCUGAAGGA | UCCUUCAGGGUUCUUUAUU | | [491-509] |
| 470 | GUUUCAAUUCUUUACACCA | UGGUGUAAAGAAUUGAAAC | | [3713-3731] |
| 471 | GUGACCAGAUCUAGCCCAU | AUGGGCUAGAUCUGGUCAC | | [2060-2078] |
| 472 | AGAGAGUCUCUAAAUCACU | AGUGAUUUAGAGACUCUCU | | [3017-3035] |
| 473 | AAAGGGAAAGGAUCAAAAG | CUUUUGAUCCUUUCCCUUU | | [2011-2029] |
| 474 | AUGAGGUGGUGAUGUUAGU | ACUAACAUCACCACCUCAU | Rat, Ms, Rb | [1216-1234] |
| 475 | GAUUUCUUAAAGUUUGAUA | UAUCAAACUUUAAGAAAUC | | [3320-3338] |
| 476 | UUCUUAUUUGAAGCAUGAA | UUCAUGCUUCAAAUAAGAA | | [2634-2652] |
| 477 | AGUUAUGAAAGUAGAGAGA | UCUCUCUACUUUCAUAACU | | [3873-3891] |
| 478 | CUGAGUAAACAAAGCAUCU | AGAUGCUUUGUUUACUCAG | | [1650-1668] |
| 479 | CUUUCUAACCUAUGUGCAU | AUGCACAUAGGUUAGAAAG | | [3785-3803] |
| 480 | UUUGCUCGAAAGAGAAUAA | UUAUUCUCUUUCGAGCAAA | | [477-495] |
| 481 | GAGAGUGUUUCUCCAAGGU | ACCUUGGAGAAACACUCUC | | [3832-3850] |
| 482 | AAAGAGGGUUGGAGGUGGA | UCCACCUCCAACCCUCUUU | | [2654-2672] |
| 483 | GAUCAUUAGGACAUAAUAC | GUAUUAUGUCCUAAUGAUC | | [2243-2261] |
| 484 | UUAUGAAACUGUGACCAGA | UCUGGUCACAGUUUCAUAA | | [2050-2068] |
| 485 | UCAUCUAUUCAACAGAUAU | AUAUCUGUUGAAUAGAUGA | | [3657-3675] |
| 486 | GGUUUAUUUGUAUUAUAUC | GAUAUAAUACAAAUAAACC | | [2903-2921] |
| 487 | CUAAAGAAAUAACUUCUAU | AUAGAAGUUAUUUCUUUAG | | [2523-2541] |
| 488 | CAAGAGUUCGAAGACAACU | AGUUGUCUUCGAACUCUUG | | [277-295] |
| 489 | ACAAAUAUGUAUCUGAUAU | AUAUCAGAUACAUAUUUGU | | [2805-2823] |
| 490 | AAAUCUGGUCACAAAUUUU | AAAAUUUGUGACCAGAUUU | | [3412-3430] |
| 491 | UGAACUAAAUAAGAACUCA | UGAGUUCUUAUUUAGUUCA | | [3463-3481] |
| 492 | AGUAAACAAAGCAUCUCCA | UGGAGAUGCUUUGUUUACU | | [1653-1671] |
| 493 | CUAUGAAGGUUUUUCUUAG | CUAAGAAAAACCUUCAUAG | | [4107-4125] |
| 494 | UAUAAUUGAAGGAGGUAUA | UAUACCUCCUUCAAUUAUA | | [3348-3366] |
| 495 | ACUUAGUAUUUAGCAUUUA | UAAAUGCUAAAUACUAAGU | | [4041-4059] |
| 496 | GAGCGGAUACUCAAGUAAG | CUUACUUGAGUAUCCGCUC | | [2426-2444] |
| 497 | UGUGGAGCUCUAUGGUUUU | AAAACCAUAGAGCUCCACA | | [1899-1917] |
| 498 | CAGAUCUGCUGCAACUGCU | AGCAGUUGCAGCAGAUCUG | | [1378-1396] |
| 499 | CAGAUGUUCUUUCUACAGA | UCUGUAGAAAGAACAUCUG | | [2347-2365] |
| 500 | UACUGAGCUGCCUCAGAAU | AUUCUGAGGCAGCUCAGUA | | [2109-2127] |

TABLE A7
NOX5 19-mer 19-mer oligomer pairs to NADPH oxidase 5

| | | | | |
|---|---|---|---|---|
| | | | | Human-20127623 |
| No. | Sense siRNA | AntiSense siRNA | Other Sp | ORF: 302-2545 |

| | | | |
|---|---|---|---|
| 1 | CCCUAUUUGACUCCGAUAG | CUAUCGGAGUCAAAUAGGG | [447-465 |
| 2 | CUAUUUGACUCCGAUAGAA | UUCUAUCGGAGUCAAAUAG | [449-467 |
| 3 | AGAUACUUUUGGAGAAACA | UGUUUCUCCAAAAGUAUCU | [1902-1920 |
| 4 | CUGAGAUACUUUUGGAGAA | UUCUCCAAAAGUAUCUCAG | [1899-1917 |
| 5 | CCCUUUUUUUCACUAUAGA | UCUAUAGUGAAAAAAAGGG | [1645-1663 |
| 6 | GUGCCUGGAAUCUUGUUUU | AAAACAAGAUUCCAGGCAC | [1520-1538 |
| 7 | GAACUUUGUACUCCAGGCU | AGCCUGGAGUACAAAGUUC | [1249-1267 |
| 8 | GAGAUACUUUUGGAGAAAC | GUUUCUCCAAAAGUAUCUC | [1901-1919 |
| 9 | GGCUGUAUGAGUCCUUCAA | UUGAAGGACUCAUACAGCC | [1797-1815 |
| 10 | AGGGCUCUGAGAUACUUUU | AAAAGUAUCUCAGAGCCCU | [1893-1911 |
| 11 | GAAUCUUGUUUUUCCUGGA | UCCAGGAAAAACAAGAUUC | [1527-1545 |
| 12 | GGUGCCUGGAAUCUUGUUU | AAACAAGAUUCCAGGCACC | [1519-1537 |
| 13 | ACACUGUGAACUUUGUACU | AGUACAAAGUUCACAGUGU | [1242-1260 |
| 14 | UGCUGACUAAACUGGAGAU | AUCUCCAGUUUAGUCAGCA | [2202-2220 |
| 15 | CCUAUUUGACUCCGAUAGA | UCUAUCGGAGUCAAAUAGG | [448-466 |
| 16 | GCAGUUUAAGACCAUUGCA | UGCAAUGGUCUUAAACUGC | [346-364 |
| 17 | GCUGACUAAACUGGAGAUG | CAUCUCCAGUUUAGUCAGC | [2203-2221 |
| 18 | AGAAAAGAAAGCAUACUUG | CAAGUAUGCUUUCUUUUCU | [2070-2088 |
| 19 | UACUUUUGGAGAAACACAA | UUGUGUUUCUCCAAAAGUA | [1905-1923 |
| 20 | CCAAGAGAAUUUCUAGCCU | AGGCUAGAAAUUCUCUUGG | [2530-2548 |
| 21 | CCAUUCUGCAGAGUAUCAU | AUGAUACUCUGCAGAAUGG | [2040-2058 |
| 22 | GGGCUCUGAGAUACUUUUG | CAAAAGUAUCUCAGAGCCC | [1894-1912 |
| 23 | GGACAAACAGGCUGUAUGA | UCAUACAGCCUGUUUGUCC | [1788-1806 |
| 24 | CCCAUGGACAAACUCAAAU | AUUUGAGUUUGUCCAUGGG | [527-545 |
| 25 | UGCAUGUGAAAGAGUCCUU | AAGGACUCUUUCACAUGCA | [408-426 |
| 26 | CAGCAGUUUAAGACCAUUG | CAAUGGUCUUAAACUGCUG | [344-362 |
| 27 | UCAUGUUGUAUCUGGAAUU | AAUUCCAGAUACAACAUGA | [228-246] |
| 28 | ACAGGCACCAGAAAAGAAA | UUUCUUUUCUGGUGCCUGU | [2061-2079 |
| 29 | GGCUCUGAGAUACUUUUGG | CCAAAAGUAUCUCAGAGCC | [1895-1913 |
| 30 | CCAAGGGCUCUGAGAUACU | AGUAUCUCAGAGCCCUUGG | [1890-1908 |
| 31 | UCAGCCUGCAAGAAUUCAA | UUGAAUUCUUGCAGGCUGA | [381-399 |
| 32 | UGGACUUUAUCUGGAUCAA | UUGAUCCAGAUAAAGUCCA | [2148-2166 |
| 33 | AGGUCACUCAUCUCCUCAU | AUGAGGAGAUGAGUGACCU | [1617-1635 |
| 34 | CCGUGUGCAUCAUGGAAGU | ACUUCCAUGAUGCACACGG | [1581-1599 |
| 35 | CAGCCUGCAAGAAUUCAAA | UUUGAAUUCUUGCAGGCUG | [382-400 |
| 36 | GGGCCAUUGUGAGAAGUUC | GAACUUCUCACAAUGGCCC | [2497-2515 |
| 37 | AGCAAGGUGUUCCAGAAAG | CUUUCUGGAACACCUUGCU | [2408-2426 |
| 38 | CUGCUGACUAAACUGGAGA | UCUCCAGUUUAGUCAGCAG | [2201-2219 |
| 39 | CCUGCUGACUAAACUGGAG | CUCCAGUUUAGUCAGCAGG | [2200-2218 |
| 40 | GGACUUUAUCUGGAUCAAC | GUUGAUCCAGAUAAAGUCC | [2149-2167 |
| 41 | AGGUGGACUUUAUCUGGAU | AUCCAGAUAAAGUCCACCU | [2145-2163 |
| 42 | GUCAUGUUGUAUCUGGAAU | AUUCCAGAUACAACAUGAC | [227-245] |
| 43 | CUCUGAGAUACUUUUGGAG | CUCCAAAAGUAUCUCAGAG | [1897-1915 |
| 44 | AGAGUCAAAGGUCGUCCAA | UUGGACGACCUUUGACUCU | [1875-1893 |
| 45 | UGCCUGGAAUCUUGUUUUU | AAAAACAAGAUUCCAGGCA | [1521-1539 |
| 46 | UACCACUGGACCAGAACAU | AUGUUCUGGUCCAGUGGUA | [1161-1179 |
| 47 | ACAAACUCAAAUUCCUCUU | AAGAGGAAUUUGAGUUUGU | [534-552 |
| 48 | UGGGCAAGAAUGACAUGAA | UUCAUGUCAUUCUUGCCCA | [2286-2304 |
| 49 | GCAGUCAUGUUGUAUCUGG | CCAGAUACAACAUGACUGC | [224-242] |
| 50 | GACAAUGAGAAAGAGUCAA | UUGACUCUUUCUCAUUGUC | [1864-1882 |
| 51 | GCUGUAUGAGUCCUUCAAG | CUUGAAGGACUCAUACAGC | [1798-1816 |
| 52 | UGGACAAACAGGCUGUAUG | CAUACAGCCUGUUUGUCCA | [1787-1805 |
| 53 | CGAGGUCUCAAUCCAUUGC | GCAAUGGAUUGAGACCUCG | [191-209] |
| 54 | CCUUUUUUUCACUAUAGAC | GUCUAUAGUGAAAAAAAGG | [1646-1664 |
| 55 | UGUGCAUCAUGGAAGUCAA | UUGACUUCCAUGAUGCACA | [1584-1602 |
| 56 | UCAUCUUUCAUGGGCCCAA | UUGGGCCCAUGAAAGAUGA | [1482-1500 |
| 57 | UGCUCCUCCUCAUGUUCAU | AUGAACAUGAGGAGGAGCA | [1377-1395 |
| 58 | GCAAGAAUUCAAAGCAGCU | AGCUGCUUUGAAUUCUUGC | [388-406 |
| 59 | AGCGGUCUUUCGAGUGGUU | AACCACUCGAAAGACCGCU | [2175-2193 |
| 60 | CCAGAAAAGAAAGCAUACU | AGUAUGCUUUCUUUUCUGG | [2068-2086 |
| 61 | CAAUGAGAAAGAGUCAAAG | CUUUGACUCUUUCUCAUUG | [1866-1884 |
| 62 | CUGGUAUGCAGUCAUGUUG | CAACAUGACUGCAUACCAG | [217-235] |
| 63 | AGGAGUGUGACAAUGAGAA | UUCUCAUUGUCACACUCCU | [1856-1874 |
| 64 | GUGGACAAACAGGCUGUAU | AUACAGCCUGUUUGUCCAC | [1786-1804 |
| 65 | CCAAGGUCACUCAUCUCCU | AGGAGAUGAGUGACCUUGG | [1614-1632 |
| 66 | UGCUGCUCCUCCUCAUGUU | AACAUGAGGAGGAGCAGCA | [1374-1392 |
| 67 | CAAGAAUUCAAAGCAGCUC | GAGCUGCUUUGAAUUCUUG | [389-407 |
| 68 | AGGGCCAUUGUGAGAAGUU | AACUUCUCACAAUGGCCCU | [2496-2514 |
| 69 | UGAAGGGCCAUUGUGAGAA | UUCUCACAAUGGCCCUUCA | [2493-2511 |
| 70 | GUGGACUUUAUCUGGAUCA | UGAUCCAGAUAAAGUCCAC | [2147-2165 |
| 71 | UCCAAGACAACAUGAAGCU | AGCUUCAUGUUGUCUUGGA | [2121-2139 |
| 72 | CAGAAAAGAAAGCAUACUU | AAGUAUGCUUUCUUUUCUG | [2069-2087 |
| 73 | GCACCAGAAAAGAAAGCAU | AUGCUUUCUUUUCUGGUGC | [2065-2083 |
| 74 | GCUUCCAUUCUGCAGAGUA | UACUCUGCAGAAUGGAAGC | [2036-2054 |
| 75 | GCUACAUCGAUGGGCCUUA | UAAGGCCCAUCGAUGUAGC | [1941-1959 |
| 76 | CAAACAGGCUGUAUGAGUC | GACUCAUACAGCCUGUUUG | [1791-1809 |
| 77 | ACACUAUCUGGCUGCACAU | AUGUGCAGCCAGAUAGUGU | [1752-1770 |
| 78 | CUGAGCAGAAAGACACUAU | AUAGUGUCUUUCUGCUCAG | [1740-1758 |
| 79 | CCUGAGCAGAAAGACACUA | UAGUGUCUUUCUGCUCAGG | [1739-1757 |
| 80 | GACUACUUGUAUCUGAACA | UGUUCAGAUACAAGUAGUC | [1670-1688 |
| 81 | CCGAGGUCUCAAUCCAUUG | CAAUGGAUUGAGACCUCGG | [190-208] |
| 82 | ACCUGGUGACUACUUGUAU | AUACAAGUAGUCACCAGGU | [1663-1681 |
| 83 | AGGUGUAUGACAUCGAUGU | ACAUCGAUGUCAUACACCU | [555-573 |
| 84 | UGGGUGACUCAGCAGUUUA | UAAACUGCUGAGUCACCCA | [335-353 |
| 85 | AGAAUUUCUAGCCUCACCU | AGGUGAGGCUAGAAAUUCU | [2535-2553 |
| 86 | AAGUUCGGCUUCAGAUUUU | AAAAUCUGAAGCCGAACUU | [2510-2528 |
| 87 | GCCAUUGUGAGAAGUUCGG | CCGAACUUCUCACAAUGGC | [2499-2517 |
| 88 | GCAAGAAUGACAUGAAGGC | GCCUUCAUGUCAUUCUUGC | [2289-2307 |
| 89 | GGUGGACUUUAUCUGGAUC | GAUCCAGAUAAAGUCCACC | [2146-2164 |
| 90 | AGAGUAUCAUGUACAGGCA | UGCCUGUACAUGAUACUCU | [2049-2067 |
| 91 | CAGUCAUGUUGUAUCUGGA | UCCAGAUACAACAUGACUG | [225-243] |
| 92 | GUAUGCAGUCAUGUUGUAU | AUACAACAUGACUGCAUAC | [220-238] |
| 93 | GUGUGACAAUGAGAAAGAG | CUCUUUCUCAUUGUCACAC | [1860-1878 |
| 94 | GGUUCUAAGAGGCUGUCGA | UCGACAGCCUCUUAGAACC | [1838-1856 |
| 95 | CGUGGUUCUAAGAGGCUGU | ACAGCCUCUUAGAACCACG | [1835-1853 |
| 96 | AGUGGACAAACAGGCUGUA | UACAGCCUGUUUGUCCACU | [1785-1803 |
| 97 | GACACUAUCUGGCUGCACA | UGUGCAGCCAGAUAGUGUC | [1751-1769 |
| 98 | GUGUGCAUCAUGGAAGUCA | UGACUUCCAUGAUGCACAC | [1583-1601 |
| 99 | AGGUGUUCUAUUGGACUCA | UGAGUCCAAUAGAACACCU | [1434-1452 |
| 100 | GAGGUGUUCUAUUGGACUC | GAGUCCAAUAGAACACCUC | [1433-1451 |
| 101 | CUCCUCCUCAUGUUCAUCU | AGAUGAACAUGAGGAGGAG | [1379-1397 |
| 102 | UCCAGGUGUAUGACAUCGA | UCGAUGUCAUACACCUGGA | [552-570 |
| 103 | UCCGAUAGAAGUGGCACCA | UGGUGCCACUUCUAUCGGA | [458-476 |
| 104 | CGAUUCUUUGCCCUAUUUG | CAAAUAGGGCAAAGAAUCG | [437-455 |
| 105 | UCAGCAGUUUAAGACCAUU | AAUGGUCUUAAACUGCUGA | [343-361 |
| 106 | AGCCCUUAGUCCUUCUAGU | ACUAGAAGGACUAAGGGCU | [134-152] |
| 107 | AGUUCGGCUUCAGAUUUUU | AAAAAUCUGAAGCCGAACU | [2511-2529 |
| 108 | CACUGGGCAAGAAUGACAU | AUGUCAUUCUUGCCCAGUG | [2283-2301 |
| 109 | UGCAGAGUAUCAUGUACAG | CUGUACAUGAUACUCUGCA | [2046-2064 |
| 110 | CUGCAGAGUAUCAUGUACA | UGUACAUGAUACUCUGCAG | [2045-2063 |
| 111 | CUUUUGGAGAAACACAAAU | AUUUGUGUUUCUCCAAAAG | [1907-1925 |
| 112 | AGUGUGACAAUGAGAAAGA | UCUUUCUCAUUGUCACACU | [1859-1877 |
| 113 | GUGGUUCUAAGAGGCUGUC | GACAGCCUCUUAGAACCAC | [1836-1854 |
| 114 | CACUAUCUGGCUGCACAUU | AAUGUGCAGCCAGAUAGUG | [1753-1771 |
| 115 | GCCUGGAAUCUUGUUUUUC | GAAAAACAAGAUUCCAGGC | [1522-1540 |
| 116 | UCCUCCUCAUGUUCAUCUG | CAGAUGAACAUGAGGAGGA | [1380-1398 |
| 117 | UGACAUCGAUGUGUGUGCA | UGCACACACAUCGAUGUCA | [562-580 |
| 118 | GUGUAUGACAUCGAUGUGU | ACACAUCGAUGUCAUACAC | [557-575 |
| 119 | UCUUUGCAGAGCGAUUCUU | AAGAAUCGCUCUGCAAAGA | [426-444 |
| 120 | CCCUUAGUCCUUCUAGUUG | CAACUAGAAGGACUAAGGG | [136-154] |
| 121 | CUCAGCAGUUUAAGACCAU | AUGGUCUUAAACUGCUGAG | [342-360 |
| 122 | GUAUCUGGAAUUGGUGGGU | ACCCACCAAUUCCAGAUAC | [235-253] |
| 123 | CACCAGAAAAGAAAGCAUA | UAUGCUUUCUUUUCUGGUG | [2066-2084 |
| 124 | GUCGAGGAGUGUGACAAUG | CAUUGUCACACUCCUCGAC | [1852-1870 |
| 125 | GAAAGACACUAUCUGGCUG | CAGCCAGAUAGUGUCUUUC | [1747-1765 |
| 126 | UCUUGUUUUUCCUGGAGAA | UUCUCCAGGAAAAACAAGA | [1530-1548 |
| 127 | GCUUCUGCUCAUCUUUCAU | AUGAAAGAUGAGCAGAAGC | [1474-1492 |
| 128 | GUCCUUCUAGUUGCGCUUU | AAAGCGCAACUAGAAGGAC | [142-160] |
| 129 | AGUCCUUCUAGUUGCGCUU | AAGCGCAACUAGAAGGACU | [141-159] |
| 130 | GGAGAAACACAAAUUCUGU | ACAGAAUUUGUGUUUCUCC | [1912-1930 |
| 131 | UCUUCCAGGUGUAUGACAU | AUGUCAUACACCUGGAAGA | [549-567 |
| 132 | GCCCUAUUUGACUCCGAUA | UAUCGGAGUCAAAUAGGGC | [446-464 |
| 133 | CAGUUUAAGACCAUUGCAG | CUGCAAUGGUCUUAAACUG | [347-365 |
| 134 | UGCCCUAUUUGACUCCGAU | AUCGGAGUCAAAUAGGGCA | [445-463 |
| 135 | GGCCAACAAGGAGAAGAAA | UUUCUUCUCCUUGUUGGCC | [2338-2356 |
| 136 | GCCACUUUGAGGUGUUCUA | UAGAACACCUCAAAGUGGC | [1425-1443 |
| 137 | UCCUUCUAGUUGCGCUUUU | AAAAGCGCAACUAGAAGGA | [143-161] |
| 138 | UGGCCAACAAGGAGAAGAA | UUCUUCUCCUUGUUGGCCA | [2337-2355 |
| 139 | GAGAAACACAAAUUCUGUA | UACAGAAUUUGUGUUUCUC | [1913-1931 |
| 140 | CCACCAUUGCUCGCUAUGA | UCAUAGCGAGCAAUGGUGG | [1692-1710 |
| 141 | GGCUCACACUGUGAACUUU | AAAGUUCACAGUGUGAGCC | [1237-1255 |
| 142 | AGCGAUUCUUUGCCCUAUU | AAUAGGGCAAAGAAUCGCU | [435-453 |
| 143 | UGGCUCACACUGUGAACUU | AAGUUCACAGUGUGAGCCA | [1236-1254 |
| 144 | GGAGAAGAAAGACUCCAUC | GAUGGAGUCUUUCUUCUCC | [2347-2365 |
| 145 | GCGGUCUUUCGAGUGGUUU | AAACCACUCGAAAGACCGC | [2176-2194 |
| 146 | ACAGAGACCAGCGGUCUUU | AAAGACCGCUGGUCUCUGU | [2166-2184 |
| 147 | GGAAUCUUGUUUUUCCUGG | CCAGGAAAAACAAGAUUCC | [1526-1544 |
| 148 | GCUUAUCCGAGGUCUCAAU | AUUGAGACCUCGGAUAAGC | [184-202] |
| 149 | CUGUGAACUUUGUACUCCA | UGGAGUACAAAGUUCACAG | [1245-1263 |
| 150 | CGGCUUCAGAUUUUUCCAA | UUGGAAAAAUCUGAAGCCG | [2515-2533 |
| 151 | GGGUUCUUGGUCUCACUGA | UCAGUGAGACCAAGAACCC | [250-268] |
| 152 | GUGGGUUCUUGGUCUCACU | AGUGAGACCAAGAACCCAC | [248-266] |
| 153 | GUGGCCACUUUGAGGUGUU | AACACCUCAAAGUGGCCAC | [1422-1440 |
| 154 | CCAGAACAUCCAGUUCCAC | GUGGAACUGGAUGUUCUGG | [1171-1189 |
| 155 | UCAACUUCGACUGCAGCUU | AAGCUGCAGUCGAAGUUGA | [1077-1095 |
| 156 | CCUUCUAGUUGCGCUUUUG | CAAAAGCGCAACUAGAAGG | [144-162] |
| 157 | CAAGAAUGAAGCCGCAGAC | GUCUGCGGCUUCAUUCUUG | [272-290] |
| 158 | AGGAGAAGAAAGACUCCAU | AUGGAGUCUUUCUUCUCCU | [2346-2364 |
| 159 | UGAGCCUGCUGACUAAACU | AGUUUAGUCAGCAGGCUCA | [2196-2214 |
| 160 | GGUCUUUCGAGUGGUUUGU | ACAAACCACUCGAAAGACC | [2178-2196 |
| 161 | CGAAGGUGUCCAAGACAAC | GUUGUCUUGGACACCUUCG | [2113-2131 |
| 162 | UGGAGAAACACAAAUUCUG | CAGAAUUUGUGUUUCUCCA | [1911-1929 |
| 163 | CAGGCUGGUAUGCAGUCAU | AUGACUGCAUACCAGCCUG | [213-231] |
| 164 | UCAUGUUCAUCUGCUCCAG | CUGGAGCAGAUGAACAUGA | [1386-1404 |
| 165 | CUCCUCAUGUUCAUCUGCU | AGCAGAUGAACAUGAGGAG | [1382-1400 |
| 166 | UCACACUGUGAACUUUGUA | UACAAAGUUCACAGUGUGA | [1240-1258 |
| 167 | GCGCUUUUGCUAUGGCCUU | AAGGCCAUAGCAAAAGCGC | [154-172] |
| 168 | UCCAGUUCCACCAGCUUAU | AUAAGCUGGUGGAACUGGA | [1179-1197 |
| 169 | ACAUCCAGUUCCACCAGCU | AGCUGGUGGAACUGGAUGU | [1176-1194 |
| 170 | UCAAGAAUGAAGCCGCAGA | UCUGCGGCUUCAUUCUUGA | [271-289] |
| 171 | UCAGAUUUUUCCAAGAGAA | UUCUCUUGGAAAAAUCUGA | [2520-2538 |
| 172 | CUGGAAUUGGUGGGUUCUU | AAGAACCCACCAAUUCCAG | [239-257] |
| 173 | AGCUGCAUAUGUACAUGAC | GUCAUGUACAUAUGCAGCU | [2259-2277 |
| 174 | CGGUCUUUCGAGUGGUUUG | CAAACCACUCGAAAGACCG | [2177-2195 |
| 175 | AAGGUGUCCAAGACAACAU | AUGUUGUCUUGGACACCUU | [2115-2133 |
| 176 | GGCUUAUCCGAGGUCUCAA | UUGAGACCUCGGAUAAGCC | [183-201] |
| 177 | CUCAUGUUCAUCUGCUCCA | UGGAGCAGAUGAACAUGAG | [1385-1403 |
| 178 | UCCUCAUGUUCAUCUGCUC | GAGCAGAUGAACAUGAGGA | [1383-1401 |
| 179 | CACACUGUGAACUUUGUAC | GUACAAAGUUCACAGUGUG | [1241-1259 |
| 180 | CUCACACUGUGAACUUUGU | ACAAAGUUCACAGUGUGAG | [1239-1257 |
| 181 | CGCCUAUGAAGCCCUUAGU | ACUAAGGGCUUCAUAGGCG | [125-143] |
| 182 | UGCCUCAACUUCGACUGCA | UGCAGUCGAAGUUGAGGCA | [1073-1091 |
| 183 | CCUUCUUUGCAGAGCGAUU | AAUCGCUCUGCAAAGAAGG | [423-441 |
| 184 | GCAUGUGAAAGAGUCCUUC | GAAGGACUCUUUCACAUGC | [409-427 |
| 185 | AGAAGAAAGACUCCAUCAC | GUGAUGGAGUCUUUCUUCU | [2349-2367 |
| 186 | AGCUCCAUAAGGUGGACUU | AAGUCCACCUUAUGGAGCU | [2136-2154 |
| 187 | AGGUGUCCAAGACAACAUG | CAUGUUGUCUUGGACACCU | [2116-2134 |
| 188 | GUAACAUCAAGUGCUACAU | AUGUAGCACUUGAUGUUAC | [1929-1947 |
| 189 | ACAGGCUGUAUGAGUCCUU | AAGGACUCAUACAGCCUGU | [1794-1812 |
| 190 | CAGUGCUCCUGAGCAGAAA | UUUCUGCUCAGGAGCACUG | [1732-1750 |
| 191 | CCAUUGCUCGCUAUGAGUG | CACUCAUAGCGAGCAAUGG | [1695-1713 |
| 192 | GCAUCAUGGAAGUCAACCU | AGGUUGACUUCCAUGAUGC | [1587-1605 |
| 193 | ACCAGCUUAUGGGCUACGU | ACGUAGCCCAUAAGCUGGU | [1188-1206 |
| 194 | CCAGUUCCACCAGCUUAUG | CAUAAGCUGGUGGAACUGG | [1180-1198 |
| 195 | UCCUACCACUGGACCAGAA | UUCUGGUCCAGUGGUAGGA | [1158-1176 |
| 196 | CUAGUUGCGCUUUUGCUAU | AUAGCAAAAGCGCAACUAG | [148-166] |
| 197 | ACGCCUAUGAAGCCCUUAG | CUAAGGGCUUCAUAGGCGU | [124-142] |
| 198 | CUGCAAGAAUUCAAAGCAG | CUGCUUUGAAUUCUUGCAG | [386-404 |
| 199 | CCUGCAAGAAUUCAAAGCA | UGCUUUGAAUUCUUGCAGG | [385-403 |
| 200 | GGCUUCAGAUUUUUCCAAG | CUUGGAAAAAUCUGAAGCC | [2516-2534 |
| 201 | GUGAGAAGUUCGGCUUCAG | CUGAAGCCGAACUUCUCAC | [2505-2523 |
| 202 | CUGAGAAGAAGGGCAAGGU | ACCUUGCCCUUCUUCUCAG | [2433-2451 |
| 203 | ACAAGGAGAAGAAAGACUC | GAGUCUUUCUUCUCCUUGU | [2343-2361 |
| 204 | GCAUAUGUACAUGACAUCU | AGAUGUCAUGUACAUAUGC | [2263-2281 |
| 205 | CUGCAUAUGUACAUGACAU | AUGUCAUGUACAUAUGCAG | [2261-2279 |
| 206 | GCUUCCUGGAGCUGCAUAU | AUAUGCAGCUCCAGGAAGC | [2250-2268 |
| 207 | CUAAACUGGAGAUGGACCA | UGGUCCAUCUCCAGUUUAG | [2208-2226 |
| 208 | UCGAAGGUGUCCAAGACAA | UUGUCUUGGACACCUUCGA | [2112-2130 |
| 209 | CUGGAUCGAAGGUGUCCAA | UUGGACACCUUCGAUCCAG | [2107-2125 |
| 210 | CCACUUUGAGGUGUUCUAU | AUAGAACACCUCAAAGUGG | [1426-1444 |
| 211 | GGCCACUUUGAGGUGUUCU | AGAACACCUCAAAGUGGCC | [1424-1442 |
| 212 | GCUCCAUAAGGUGGACUUU | AAAGUCCACCUUAUGGAGC | [2137-2155 |
| 213 | AGACAACAUGAAGCUCCAU | AUGGAGCUUCAUGUUGUCU | [2125-2143 |
| 214 | GUGUCCAAGACAACAUGAA | UUCAUGUUGUCUUGGACAC | [2118-2136 |
| 215 | CCCUUUGCUUCCAUUCUGC | GCAGAAUGGAAGCAAAGGG | [2030-2048 |
| 216 | AUGUUCAUCUGCUCCAGUU | AACUGGAGCAGAUGAACAU | [1388-1406 |
| 217 | UCCACCAGCUUAUGGGCUA | UAGCCCAUAAGCUGGUGGA | [1185-1203 |
| 218 | ACCAGAACAUCCAGUUCCA | UGGAACUGGAUGUUCUGGU | [1170-1188 |
| 219 | UGGACCAGAACAUCCAGUU | AACUGGAUGUUCUGGUCCA | [1167-1185 |
| 220 | UGGCUCAAGUCCUACCACU | AGUGGUAGGACUUGAGCCA | [1149-1167 |
| 221 | UCAUGGAGAACCUGACCAU | AUGGUCAGGUUCUCCAUGA | [846-864 |
| 222 | CGAUAGAAGUGGCACCAUC | GAUGGUGCCACUUCUAUCG | [460-478 |
| 223 | CCGAUAGAAGUGGCACCAU | AUGGUGCCACUUCUAUCGG | [459-477 |
| 224 | CAAGAGAAUUUCUAGCCUC | GAGGCUAGAAAUUCUCUUG | [2531-2549 |
| 225 | GAGAAGUUCGGCUUCAGAU | AUCUGAAGCCGAACUUCUC | [2507-2525 |
| 226 | GUGCAGGUCUUCUUCUGUG | CACAGAAGAAGACCUGCAC | [2450-2468 |
| 227 | UGGAGCUGCAUAUGUACAU | AUGUACAUAUGCAGCUCCA | [2256-2274 |
| 228 | UCCAUAAGGUGGACUUUAU | AUAAAGUCCACCUUAUGGA | [2139-2157 |
| 229 | ACUACUUGUAUCUGAACAU | AUGUUCAGAUACAAGUAGU | [1671-1689 |
| 230 | CCUGGUGACUACUUGUAUC | GAUACAAGUAGUCACCAGG | [1664-1682 |
| 231 | UCACUAUAGACCUGGUGAC | GUCACCAGGUCUAUAGUGA | [1654-1672 |
| 232 | CUGUGGCUCACACUGUGAA | UUCACAGUGUGAGCCACAG | [1233-1251 |
| 233 | AGAACAUCCAGUUCCACCA | UGGUGGAACUGGAUGUUCU | [1173-1191 |
| 234 | UCAAGUCCUACCACUGGAC | GUCCAGUGGUAGGACUUGA | [1153-1171 |
| 235 | CUCAAGUCCUACCACUGGA | UCCAGUGGUAGGACUUGAG | [1152-1170 |
| 236 | AGAGCGAUUCUUUGCCCUA | UAGGGCAAAGAAUCGCUCU | [433-451 |
| 237 | GUCCUUCUUUGCAGAGCGA | UCGCUCUGCAAAGAAGGAC | [421-439 |
| 238 | AAGCAGCUCUGCAUGUGAA | UUCACAUGCAGAGCUGCUU | [399-417 |
| 239 | CAAAGCAGCUCUGCAUGUG | CACAUGCAGAGCUGCUUUG | [397-415 |
| 240 | AGAAUUCAAAGCAGCUCUG | CAGAGCUGCUUUGAAUUCU | [391-409 |
| 241 | GCCUGCAAGAAUUCAAAGC | GCUUUGAAUUCUUGCAGGC | [384-402 |
| 242 | GAAGCCCUUAGUCCUUCUA | UAGAAGGACUAAGGGCUUC | [132-150] |
| 243 | GCCUGCUGACUAAACUGGA | UCCAGUUUAGUCAGCAGGC | [2199-2217 |
| 244 | UCAUGUACAGGCACCAGAA | UUCUGGUGCCUGUACAUGA | [2055-2073 |
| 245 | GUAUCAUGUACAGGCACCA | UGGUGCCUGUACAUGAUAC | [2052-2070 |
| 246 | CUGGUGACUACUUGUAUCU | AGAUACAAGUAGUCACCAG | [1665-1683 |
| 247 | CAAGGUCACUCAUCUCCUC | GAGGAGAUGAGUGACCUUG | [1615-1633 |
| 248 | GGCUUCUGCUCAUCUUUCA | UGAAAGAUGAGCAGAAGCC | [1473-1491 |
| 249 | UGUGGCUUCUGCUCAUCUU | AAGAUGAGCAGAAGCCACA | [1470-1488 |
| 250 | GGUGUUCUAUUGGACUCAC | GUGAGUCCAAUAGAACACC | [1435-1453 |
| 251 | CGCUUUUGCUAUGGCCUUC | GAAGGCCAUAGCAAAAGCG | [155-173] |
| 252 | GGACCAGAACAUCCAGUUC | GAACUGGAUGUUCUGGUCC | [1168-1186 |
| 253 | UGGAGAACCUGACCAUCAG | CUGAUGGUCAGGUUCUCCA | [849-867 |
| 254 | CUCUGCAUGUGAAAGAGUC | GACUCUUUCACAUGCAGAG | [405-423 |
| 255 | CAGAUUUUUCCAAGAGAAU | AUUCUCUUGGAAAAAUCUG | [2521-2539 |
| 256 | UCCUGGCCAACAAGGAGAA | UUCUCCUUGUUGGCCAGGA | [2334-2352 |
| 257 | UGGUUUGUGAGCCUGCUGA | UCAGCAGGCUCACAAACCA | [2189-2207 |
| 258 | UCGAGUGGUUUGUGAGCCU | AGGCUCACAAACCACUCGA | [2184-2202 |
| 259 | GCCUAUGAAGCCCUUAGUC | GACUAAGGGCUUCAUAGGC | [126-144] |
| 260 | GGAGUCAUGGAGAACCUGA | UCAGGUUCUCCAUGACUCC | [842-860 |
| 261 | GGACAAACUCAAAUUCCUC | GAGGAAUUUGAGUUUGUCC | [532-550 |
| 262 | UGGACAAACUCAAAUUCCU | AGGAAUUUGAGUUUGUCCA | [531-549 |
| 263 | GUGAAAGAGUCCUUCUUUG | CAAAGAAGGACUCUUUCAC | [413-431 |
| 264 | AUGUGAAAGAGUCCUUCUU | AAGAAGGACUCUUUCACAU | [411-429 |
| 265 | AGCUCUGCAUGUGAAAGAG | CUCUUUCACAUGCAGAGCU | [403-421 |
| 266 | GAAGUUCGGCUUCAGAUUU | AAAUCUGAAGCCGAACUUC | [2509-2527 |
| 267 | UCCAGAAAGUGGCUGCUGA | UCAGCAGCCACUUUCUGGA | [2418-2436 |
| 268 | UGCAUAUGUACAUGACAUC | GAUGUCAUGUACAUAUGCA | [2262-2280 |
| 269 | GCUGCAUAUGUACAUGACA | UGUCAUGUACAUAUGCAGC | [2260-2278 |
| 270 | CCAUAAGGUGGACUUUAUC | GAUAAAGUCCACCUUAUGG | [2140-2158 |
| 271 | CCAAGACAACAUGAAGCUC | GAGCUUCAUGUUGUCUUGG | [2122-2140 |
| 272 | UGCUUCCAUUCUGCAGAGU | ACUCUGCAGAAUGGAAGCA | [2035-2053 |
| 273 | CAGGCUGUAUGAGUCCUUC | GAAGGACUCAUACAGCCUG | [1795-1813 |
| 274 | UCAUGGAAGUCAACCUCCU | AGGAGGUUGACUUCCAUGA | [1590-1608 |
| 275 | CUGGAAUCUUGUUUUUCCU | AGGAAAAACAAGAUUCCAG | [1524-1542 |
| 276 | CCAACUUCUGGAAGUGGCU | AGCCACUUCCAGAAGUUGG | [1497-1515 |
| 277 | CACUUUGAGGUGUUCUAUU | AAUAGAACACCUCAAAGUG | [1427-1445 |
| 278 | UGGCCACUUUGAGGUGUUC | GAACACCUCAAAGUGGCCA | [1423-1441 |
| 279 | UGUGAAAGAGUCCUUCUUU | AAAGAAGGACUCUUUCACA | [412-430 |
| 280 | GCAGCUCUGCAUGUGAAAG | CUUUCACAUGCAGAGCUGC | [401-419 |
| 281 | AGAAGUUCGGCUUCAGAUU | AAUCUGAAGCCGAACUUCU | [2508-2526 |
| 282 | UGAAGCCCUUAGUCCUUCU | AGAAGGACUAAGGGCUUCA | [131-149] |
| 283 | CAGGUCUUCUUCUGUGGCU | AGCCACAGAAGAAGACCUG | [2453-2471 |
| 284 | UUGACCUCCUGGCCAACAA | UUGUUGGCCAGGAGGUCAA | [2328-2346 |
| 285 | CUCCAUAAGGUGGACUUUA | UAAAGUCCACCUUAUGGAG | [2138-2156 |
| 286 | CAGGAUCUUUGCCUCUGAG | CUCAGAGGCAAAGAUCCUG | [1975-1993 |
| 287 | GGCUGGUAUGCAGUCAUGU | ACAUGACUGCAUACCAGCC | [215-233] |
| 288 | UGAGCAGAAAGACACUAUC | GAUAGUGUCUUUCUGCUCA | [1741-1759 |
| 289 | GUGCUCCUGAGCAGAAAGA | UCUUUCUGCUCAGGAGCAC | [1734-1752 |
| 290 | UGACUACUUGUAUCUGAAC | GUUCAGAUACAAGUAGUCA | [1669-1687 |
| 291 | GACAAACUCAAAUUCCUCU | AGAGGAAUUUGAGUUUGUC | [533-551 |
| 292 | AGCAGCUCUGCAUGUGAAA | UUUCACAUGCAGAGCUGCU | [400-418 |
| 293 | GCUUCAGAUUUUUCCAAGA | UCUUGGAAAAAUCUGAAGC | [2517-2535 |
| 294 | GUACAUGACAUCUGCACUG | CAGUGCAGAUGUCAUGUAC | [2269-2287 |
| 295 | GGAUCUUUGCCUCUGAGCA | UGCUCAGAGGCAAAGAUCC | [1977-1995 |
| 296 | GUGACAAUGAGAAAGAGUC | GACUCUUUCUCAUUGUCAC | [1862-1880 |
| 297 | CUGUCGAGGAGUGUGACAA | UUGUCACACUCCUCGACAG | [1850-1868 |
| 298 | CCUGGAAUCUUGUUUUUCC | GGAAAAACAAGAUUCCAGG | [1523-1541 |
| 299 | UCGUGUGGCUUCUGCUCAU | AUGAGCAGAAGCCACACGA | [1467-1485 |
| 300 | UCCUCUUCCAGGUGUAUGA | UCAUACACCUGGAAGAGGA | [546-564 |
| 301 | AGGUGCAGGUCUUCUUCUG | CAGAAGAAGACCUGCACCU | [2448-2466 |
| 302 | UGACAUCUGCACUGGGCAA | UUGCCCAGUGCAGAUGUCA | [2274-2292 |
| 303 | GAAGCUCCAUAAGGUGGAC | GUCCACCUUAUGGAGCUUC | [2134-2152 |
| 304 | UGAAGCUCCAUAAGGUGGA | UCCACCUUAUGGAGCUUCA | [2133-2151 |
| 305 | UGGUCUCACUGACUUCAAG | CUUGAAGUCAGUGAGACCA | [257-275] |
| 306 | GUGGCUUCUGCUCAUCUUU | AAAGAUGAGCAGAAGCCAC | [1471-1489 |
| 307 | GUCUCACUGACUUCAAGAA | UUCUUGAAGUCAGUGAGAC | [259-277] |
| 308 | UUCUUGGUCUCACUGACUU | AAGUCAGUGAGACCAAGAA | [253-271] |
| 309 | CUUCAGAUUUUUCCAAGAG | CUCUUGGAAAAAUCUGAAG | [2518-2536 |
| 310 | GCCAACAAGGAGAAGAAAG | CUUUCUUCUCCUUGUUGGC | [2339-2357 |
| 311 | GGCACCAGAAAAGAAAGCA | UGCUUUCUUUUCUGGUGCC | [2064-2082 |
| 312 | CUCACUGACUUCAAGAAUG | CAUUCUUGAAGUCAGUGAG | [261-279] |
| 313 | AGAAUGACAUGAAGGCCAU | AUGGCCUUCAUGUCAUUCU | [2292-2310 |
| 314 | CACUGACUUCAAGAAUGAA | UUCAUUCUUGAAGUCAGUG | [263-281] |
| 315 | UUCAGAUUUUUCCAAGAGA | UCUCUUGGAAAAAUCUGAA | [2519-2537 |
| 316 | GGUUCUUGGUCUCACUGAC | GUCAGUGAGACCAAGAACC | [251-269] |
| 317 | GAAUGACAUGAAGGCCAUU | AAUGGCCUUCAUGUCAUUC | [2293-2311 |
| 318 | UCAAAUUCCUCUUCCAGGU | ACCUGGAAGAGGAAUUUGA | [540-558 |
| 319 | CUGCUGAGAAGAAGGGCAA | UUGCCCUUCUUCUCAGCAG | [2430-2448 |
| 320 | AGCAGAAAGACACUAUCUG | CAGAUAGUGUCUUUCUGCU | [1743-1761 |

TABLE A8
NOX5 - 19-mer oligomer pairs NADPH oxidase 5

| | | | | |
|---|---|---|---|---|
| | | | Other | Human-20127623 |
| No. | Sense siRNA | AntiSense siRNA | Sp | ORF: 302-2545 |

| | | | |
|---|---|---|---|
| 1 | GGGUGACUCAGCAGUUUAA | UUAAACUGCUGAGUCACCC | [336-354] |
| 2 | GGAGCAAGGUGUUCCAGAA | UUCUGGAACACCUUGCUCC | [2406-2424] |
| 3 | CCAGGUGUAUGACAUCGAU | AUCGAUGUCAUACACCUGG | [553-571] |
| 4 | CACAAAUUCUGUAACAUCA | UGAUGUUACAGAAUUUGUG | [1919-1937] |
| 5 | ACAAAUUCUGUAACAUCAA | UUGAUGUUACAGAAUUUGU | [1920-1938] |
| 6 | GGAGUGUGACAAUGAGAAA | UUUCUCAUUGUCACACUCC | [1857-1875] |
| 7 | UGACUAAACUGGAGAUGGA | UCCAUCUCCAGUUUAGUCA | [2205-2223] |
| 8 | GAUUCUUUGCCCUAUUUGA | UCAAAUAGGGCAAAGAAUC | [438-456] |
| 9 | GUGACUACUUGUAUCUGAA | UUCAGAUACAAGUAGUCAC | [1668-1686] |
| 10 | CGAGGAGUGUGACAAUGAG | CUCAUUGUCACACUCCUCG | [1854-1872] |
| 11 | CCAACAAGGAGAAGAAAGA | UCUUUCUUCUCCUUGUUGG | [2340-2358] |
| 12 | UUGUGAGCCUGCUGACUAA | UUAGUCAGCAGGCUCACAA | [2193-2211] |
| 13 | UACGGACGACGCCUAUGAA | UUCAUAGGCGUCGUCCGUA | [116-134] |
| 14 | GUGACUCAGCAGUUUAAGA | UCUUAAACUGCUGAGUCAC | [338-356] |
| 15 | CAUGUACAGGCACCAGAAA | UUUCUGGUGCCUGUACAUG | [2056-2074] |
| 16 | GGUAUGCAGUCAUGUUGUA | UACAACAUGACUGCAUACC | [219-237] |
| 17 | GACUUUAUCUGGAUCAACA | UGUUGAUCCAGAUAAAGUC | [2150-2168] |
| 18 | GAAGGUGUCCAAGACAACA | UGUUGUCUUGGACACCUUC | [2114-2132] |
| 19 | GCUCCUGAGCAGAAAGACA | UGUCUUUCUGCUCAGGAGC | [1736-1754] |
| 20 | UCCUGGAGCUGCAUAUGUA | UACAUAUGCAGCUCCAGGA | [2253-2271] |
| 21 | ACAACAUGAAGCUCCAUAA | UUAUGGAGCUUCAUGUUGU | [2127-2145] |
| 22 | GAGCAAGGUGUUCCAGAAA | UUUCUGGAACACCUUGCUC | [2407-2425] |
| 23 | GCUCUGAGAUACUUUUGGA | UCCAAAAGUAUCUCAGAGC | [1896-1914] |
| 24 | GGAUCGAAGGUGUCCAAGA | UCUUGGACACCUUCGAUCC | [2109-2127] |
| 25 | UCUGUAACAUCAAGUGCUA | UAGCACUUGAUGUUACAGA | [1926-1944] |
| 26 | GGGCAAGAAUGACAUGAAG | CUUCAUGUCAUUCUUGCCC | [2287-2305] |
| 27 | GGAGAUCAGCCUGCAAGAA | UUCUUGCAGGCUGAUCUCC | [376-394] |
| 28 | UGAGAUACUUUUGGAGAAA | UUUCUCCAAAAGUAUCUCA | [1900-1918] |
| 29 | GCAGGAUCUUUGCCUCUGA | UCAGAGGCAAAGAUCCUGC | [1974-1992] |
| 30 | AGAAGAAGGGCAAGGUGCA | UGCACCUUGCCCUUCUUCU | [2436-2454] |
| 31 | GAGGAGUGUGACAAUGAGA | UCUCAUUGUCACACUCCUC | [1855-1873] |
| 32 | AAGACAACAUGAAGCUCCA | UGGAGCUUCAUGUUGUCUU | [2124-2142] |
| 33 | AGAUCAGCCUGCAAGAAUU | AAUUCUUGCAGGCUGAUCU | [378-396] |
| 34 | GACCAUUGCAGGAGAAGAU | AUCUUCUCCUGCAAUGGUC | [355-373] |
| 35 | UUAUCUGGAUCAACAGAGA | UCUCUGUUGAUCCAGAUAA | [2154-2172] |
| 36 | AGCCGUGUGCAUCAUGGAA | UUCCAUGAUGCACACGGCU | [1579-1597] |
| 37 | CAUCUGCACUGGGCAAGAA | UUCUUGCCCAGUGCAGAUG | [2277-2295] |
| 38 | GUCCAAGGGCUCUGAGAUA | UAUCUCAGAGCCCUUGGAC | [1888-1906] |
| 39 | GGUGACUACUUGUAUCUGA | UCAGAUACAAGUAGUCACC | [1667-1685] |
| 40 | CAGGCACCAGAAAAGAAAG | CUUUCUUUUCUGGUGCCUG | [2062-2080] |
| 41 | AGUCAUGUUGUAUCUGGAA | UUCCAGAUACAACAUGACU | [226-244] |
| 42 | CACCAUUGCUCGCUAUGAG | CUCAUAGCGAGCAAUGGUG | [1693-1711] |
| 43 | UACAGGCACCAGAAAAGAA | UUCUUUUCUGGUGCCUGUA | [2060-2078] |
| 44 | CACUGGACCAGAACAUCCA | UGGAUGUUCUGGUCCAGUG | [1164-1182] |
| 45 | GCAAGGUGUUCCAGAAAGU | ACUUUCUGGAACACCUUGC | [2409-2427] |
| 46 | GGUGUCCAAGACAACAUGA | UCAUGUUGUCUUGGACACC | [2117-2135] |
| 47 | CUGCACUGGGCAAGAAUGA | UCAUUCUUGCCCAGUGCAG | [2280-2298] |
| 48 | GUUUAAGACCAUUGCAGGA | UCCUGCAAUGGUCUUAAAC | [349-367] |
| 49 | GUACAGGCACCAGAAAAGA | UCUUUUCUGGUGCCUGUAC | [2059-2077] |
| 50 | GGUCUCACUGACUUCAAGA | UCUUGAAGUCAGUGAGACC | [258-276] |
| 51 | UGAGAAGUUCGGCUUCAGA | UCUGAAGCCGAACUUCUCA | [2506-2524] |
| 52 | GAGAAGAAAGACUCCAUCA | UGAUGGAGUCUUUCUUCUC | [2348-2366] |
| 53 | AACUCAAAUUCCUCUUCCA | UGGAAGAGGAAUUUGAGUU | [537-555] |
| 54 | CUUCAAGAAUGAAGCCGCA | UGCGGCUUCAUUCUUGAAG | [269-287] |
| 55 | UGUUGUAUCUGGAAUUGGU | ACCAAUUCCAGAUACAACA | [231-249] |
| 56 | CCUGGAGCUGCAUAUGUAC | GUACAUAUGCAGCUCCAGG | [2254-2272] |
| 57 | CAAAUUCUGUAACAUCAAG | CUUGAUGUUACAGAAUUUG | [1921-1939] |
| 58 | CAUCAAGUGCUACAUCGAU | AUCGAUGUAGCACUUGAUG | [1933-1951] |
| 59 | UCACUCAUCUCCUCAUCAA | UUGAUGAGGAGAUGAGUGA | [1620-1638] |
| 60 | GCAAGGUGCAGGUCUUCUU | AAGAAGACCUGCACCUUGC | [2445-2463] |
| 61 | GUGGGUGACUCAGCAGUUU | AAACUGCUGAGUCACCCAC | [334-352] |
| 62 | GCUGGUAUGCAGUCAUGUU | AACAUGACUGCAUACCAGC | [216-234] |
| 63 | UAAGACCAUUGCAGGAGAA | UUCUCCUGCAAUGGUCUUA | [352-370] |
| 64 | AGACCAUUGCAGGAGAAGA | UCUUCUCCUGCAAUGGUCU | [354-372] |
| 65 | GAUCGAAGGUGUCCAAGAC | GUCUUGGACACCUUCGAUC | [2110-2128] |
| 66 | GGUGACUCAGCAGUUUAAG | CUUAAACUGCUGAGUCACC | [337-355] |
| 67 | CCUCCAAGGUCACUCAUCU | AGAUGAGUGACCUUGGAGG | [1611-1629] |
| 68 | UGUCGAGGAGUGUGACAAU | AUUGUCACACUCCUCGACA | [1851-1869] |
| 69 | AGGCUGUAUGAGUCCUUCA | UGAAGGACUCAUACAGCCU | [1796-1814] |
| 70 | CUUCCAUUCUGCAGAGUAU | AUACUCUGCAGAAUGGAAG | [2037-2055] |
| 71 | GAAAGAGUCCUUCUUUGCA | UGCAAAGAAGGACUCUUUC | [415-433] |
| 72 | CUUUAUCUGGAUCAACAGA | UCUGUUGAUCCAGAUAAAG | [2152-2170] |
| 73 | GCACUGGGCAAGAAUGACA | UGUCAUUCUUGCCCAGUGC | [2282-2300] |
| 74 | ACAUCAAGUGCUACAUCGA | UCGAUGUAGCACUUGAUGU | [1932-1950] |
| 75 | GAGCGAUUCUUUGCCCUAU | AUAGGGCAAAGAAUCGCUC | [434-452] |
| 76 | UCACUGACUUCAAGAAUGA | UCAUUCUUGAAGUCAGUGA | [262-280] |
| 77 | CAAGGGCUCUGAGAUACUU | AAGUAUCUCAGAGCCCUUG | [1891-1909] |
| 78 | AAGAGAAUUUCUAGCCUCA | UGAGGCUAGAAAUUCUCUU | [2532-2550]+ |
| 79 | UGUCCAAGACAACAUGAAG | CUUCAUGUUGUCUUGGACA | [2119-2137] |
| 80 | GCGAUUCUUUGCCCUAUUU | AAAUAGGGCAAAGAAUCGC | [436-454] |
| 81 | ACAUGAAGCUCCAUAAGGU | ACCUUAUGGAGCUUCAUGU | [2130-2148] |
| 82 | UGACAAUGAGAAAGAGUCA | UGACUCUUUCUCAUUGUCA | [1863-1881] |
| 83 | CUACCACUGGACCAGAACA | UGUUCUGGUCCAGUGGUAG | [1160-1178] |
| 84 | CCAUGGACAAACUCAAAUU | AAUUUGAGUUUGUCCAUGG | [528-546] |
| 85 | UUGGUCUCACUGACUUCAA | UUGAAGUCAGUGAGACCAA | [256-274] |
| 86 | AUGUACAGGCACCAGAAAA | UUUUCUGGUGCCUGUACAU | [2057-2075] |
| 87 | GACCUGGUGACUACUUGUA | UACAAGUAGUCACCAGGUC | [1662-1680] |
| 88 | CAACAUGAAGCUCCAUAAG | CUUAUGGAGCUUCAUGUUG | [2128-2146] |
| 89 | CAUCCAGUUCCACCAGCUU | AAGCUGGUGGAACUGGAUG | [1177-1195] |
| 90 | UGUGAGCCUGCUGACUAAA | UUUAGUCAGCAGGCUCACA | [2194-2212] |
| 91 | ACUUUGUACUCCAGGCUCA | UGAGCCUGGAGUACAAAGU | [1251-1269] |
| 92 | ACAAUGAGAAAGAGUCAAA | UUUGACUCUUUCUCAUUGU | [1865-1883] |
| 93 | UCGGCUUCAGAUUUUUCCA | UGGAAAAAUCUGAAGCCGA | [2514-2532] |
| 94 | CUGACUGGAGCAAGGUGUU | AACACCUUGCUCCAGUCAG | [2400-2418] |
| 95 | ACUGGAGCAAGGUGUUCCA | UGGAACACCUUGCUCCAGU | [2403-2421] |
| 96 | GAGUCAUGGAGAACCUGAC | GUCAGGUUCUCCAUGACUC | [843-861] |
| 97 | CAGCUCUGCAUGUGAAAGA | UCUUUCACAUGCAGAGCUG | [402-420] |
| 98 | CUGGUGCCUGGAAUCUUGU | ACAAGAUUCCAGGCACCAG | [1517-1535] |
| 99 | CUGGAGCUGCAUAUGUACA | UGUACAUAUGCAGCUCCAG | [2255-2273] |
| 100 | AACUUCGACUGCAGCUUCA | UGAAGCUGCAGUCGAAGUU | [1079-1097] |
| 101 | CUUCAAGGCAUCAGACCCA | UGGGUCUGAUGCCUUGAAG | [1810-1828] |
| 102 | CUGGAUCAACAGAGACCAG | CUGGUCUCUGUUGAUCCAG | [2158-2176] |
| 103 | AAGAGUCCUUCUUUGCAGA | UCUGCAAAGAAGGACUCUU | [417-435] |
| 104 | GCUUAUGGGCUACGUGGUA | UACCACGUAGCCCAUAAGC | [1192-1210] |
| 105 | CACUCAUCUCCUCAUCAAG | CUUGAUGAGGAGAUGAGUG | [1621-1639] |
| 106 | AAGGUGGACUUUAUCUGGA | UCCAGAUAAAGUCCACCUU | [2144-2162] |
| 107 | GAGCAGAAAGACACUAUCU | AGAUAGUGUCUUUCUGCUC | [1742-1760] |
| 108 | UGGUGCCUGGAAUCUUGUU | AACAAGAUUCCAGGCACCA | [1518-1536] |
| 109 | GAGAUCAGCCUGCAAGAAU | AUUCUUGCAGGCUGAUCUC | [377-395] |
| 110 | CUAUAGACCUGGUGACUAC | GUAGUCACCAGGUCUAUAG | [1657-1675] |
| 111 | UCUAGUUGCGCUUUUGCUA | UAGCAAAAGCGCAACUAGA | [147-165] |
| 112 | AGACCUGGUGACUACUUGU | ACAAGUAGUCACCAGGUCU | [1661-1679] |
| 113 | CUACAUCGAUGGGCCUUAU | AUAAGGCCCAUCGAUGUAG | [1942-1960] |
| 114 | ACUCAGCAGUUUAAGACCA | UGGUCUUAAACUGCUGAGU | [341-359] |
| 115 | GACGCCUAUGAAGCCCUUA | UAAGGGCUUCAUAGGCGUC | [123-141] |
| 116 | ACUUUAUCUGGAUCAACAG | CUGUUGAUCCAGAUAAAGU | [2151-2169] |
| 117 | UCUGGAUCAACAGAGACCA | UGGUCUCUGUUGAUCCAGA | [2157-2175] |
| 118 | AUCGAAGGUGUCCAAGACA | UGUCUUGGACACCUUCGAU | [2111-2129] |
| 119 | AUGGAGAACCUGACCAUCA | UGAUGGUCAGGUUCUCCAU | [848-866] |
| 120 | CUAUGAAGCCCUUAGUCCU | AGGACUAAGGGCUUCAUAG | [128-146] |
| 121 | ACCAGAAAAGAAAGCAUAC | GUAUGCUUUCUUUUCUGGU | [2067-2085] |
| 122 | GCCCUUAGUCCUUCUAGUU | AACUAGAAGGACUAAGGGC | [135-153] |
| 123 | CCUUUGCUUCCAUUCUGCA | UGCAGAAUGGAAGCAAAGG | [2031-2049] |
| 124 | UCGAGGAGUGUGACAAUGA | UCAUUGUCACACUCCUCGA | [1853-1871] |
| 125 | CAACAAGGAGAAGAAAGAC | GUCUUUCUUCUCCUUGUUG | [2341-2359] |
| 126 | GGUGCAGGUCUUCUUCUGU | ACAGAAGAAGACCUGCACC | [2449-2467] |
| 127 | UGUAACAUCAAGUGCUACA | UGUAGCACUUGAUGUUACA | [1928-1946] |
| 128 | AAUUCCUCUUCCAGGUGUA | UACACCUGGAAGAGGAAUU | [543-561] |
| 129 | UCCUGAGCAGAAAGACACU | AGUGUCUUUCUGCUCAGGA | [1738-1756] |
| 130 | GUGCAUCAUGGAAGUCAAC | GUUGACUUCCAUGAUGCAC | [1585-1603] |
| 131 | GCAGAAAGACACUAUCUGG | CCAGAUAGUGUCUUUCUGC | [1744-1762] |
| 132 | AAAGCAGCUCUGCAUGUGA | UCACAUGCAGAGCUGCUUU | [398-416] |
| 133 | GAUACUUUUGGAGAAACAC | GUGUUUCUCCAAAAGUAUC | [1903-1921] |
| 134 | UGGUAUGCAGUCAUGUUGU | ACAACAUGACUGCAUACCA | [218-236] |
| 135 | AAGAGUCAAAGGUCGUCCA | UGGACGACCUUUGACUCUU | [1874-1892] |
| 136 | CAGUGGACAAACAGGCUGU | ACAGCCUGUUUGUCCACUG | [1784-1802] |
| 137 | CAUGUGAAAGAGUCCUUCU | AGAAGGACUCUUUCACAUG | [410-428] |
| 138 | AUCCGAGGUCUCAAUCCAU | AUGGAUUGAGACCUCGGAU | [188-206] |
| 139 | GAUUUUUCCAAGAGAAUUU | AAAUUCUCUUGGAAAAAUC | [2523-2541] |
| 140 | GAGCUGCAUAUGUACAUGA | UCAUGUACAUAUGCAGCUC | [2258-2276] |
| 141 | AGUCAAAGGUCGUCCAAGG | CCUUGGACGACCUUUGACU | [1877-1895] |
| 142 | UAUCCGAGGUCUCAAUCCA | UGGAUUGAGACCUCGGAUA | [187-205] |
| 143 | UUAAGACCAUUGCAGGAGA | UCUCCUGCAAUGGUCUUAA | [351-369] |
| 144 | GUUCUAAGAGGCUGUCGAG | CUCGACAGCCUCUUAGAAC | [1839-1857] |
| 145 | AAGGGCUCUGAGAUACUUU | AAAGUAUCUCAGAGCCCUU | [1892-1910] |
| 146 | GACAACAUGAAGCUCCAUA | UAUGGAGCUUCAUGUUGUC | [2126-2144] |
| 147 | AUUUGACUCCGAUAGAAGU | ACUUCUAUCGGAGUCAAAU | [451-469] |
| 148 | CUUUGUACUCCAGGCUCAG | CUGAGCCUGGAGUACAAAG | [1252-1270] |
| 149 | UGUGAACUUUGUACUCCAG | CUGGAGUACAAAGUUCACA | [1246-1264] |
| 150 | ACCAUUGCUCGCUAUGAGU | ACUCAUAGCGAGCAAUGGU | [1694-1712] |
| 151 | UUGCCCUAUUUGACUCCGA | UCGGAGUCAAAUAGGGCAA | [444-462] |
| 152 | GAAGAAAGACUCCAUCACG | CGUGAUGGAGUCUUUCUUC | [2350-2368] |
| 153 | UGUACAGGCACCAGAAAAG | CUUUUCUGGUGCCUGUACA | [2058-2076] |
| 154 | ACUUCGACUGCAGCUUCAU | AUGAAGCUGCAGUCGAAGU | [1080-1098] |
| 155 | AUCUGCACUGGGCAAGAAU | AUUCUUGCCCAGUGCAGAU | [2278-2296] |
| 156 | ACAUCUGCACUGGGCAAGA | UCUUGCCCAGUGCAGAUGU | [2276-2294] |
| 157 | GAUCUUUGCCUCUGAGCAU | AUGCUCAGAGGCAAAGAUC | [1978-1996] |
| 158 | GGUGUAUGACAUCGAUGUG | CACAUCGAUGUCAUACACC | [556-574] |
| 159 | UUUUCCAAGAGAAUUUCUA | UAGAAAUUCUCUUGGAAAA | [2526-2544] |
| 160 | AAAUUCUGUAACAUCAAGU | ACUUGAUGUUACAGAAUUU | [1922-1940] |
| 161 | UGGAGCAAGGUGUUCCAGA | UCUGGAACACCUUGCUCCA | [2405-2423] |
| 162 | AAUUCAAAGCAGCUCUGCA | UGCAGAGCUGCUUUGAAUU | [393-411] |
| 163 | CAAGGUGUUCCAGAAAGUG | CACUUUCUGGAACACCUUG | [2410-2428] |
| 164 | GACUAAACUGGAGAUGGAC | GUCCAUCUCCAGUUUAGUC | [2206-2224] |
| 165 | GAGCCUGCUGACUAAACUG | CAGUUUAGUCAGCAGGCUC | [2197-2215] |
| 166 | CACUAUAGACCUGGUGACU | AGUCACCAGGUCUAUAGUG | [1655-1673] |
| 167 | AUGGGCCCAACUUCUGGAA | UUCCAGAAGUUGGGCCCAU | [1491-1509] |
| 168 | UCCGAGGUCUCAAUCCAUU | AAUGGAUUGAGACCUCGGA | [189-207] |
| 169 | GGAAUUGGUGGGUUCUUGG | CCAAGAACCCACCAAUUCC | [241-259] |
| 170 | CUUGGUCUCACUGACUUCA | UGAAGUCAGUGAGACCAAG | [255-273] |
| 171 | UGCUGGUGCCUGGAAUCUU | AAGAUUCCAGGCACCAGCA | [1515-1533] |
| 172 | UGUGAGAAGUUCGGCUUCA | UGAAGCCGAACUUCUCACA | [2504-2522] |
| 173 | CACUGUGAACUUUGUACUC | GAGUACAAAGUUCACAGUG | [1243-1261] |
| 174 | GAAUUGGUGGGUUCUUGGU | ACCAAGAACCCACCAAUUC | [242-260] |
| 175 | CAAGGUGCAGGUCUUCUUC | GAAGAAGACCUGCACCUUG | [2446-2464] |
| 176 | CUGGGCAAGAAUGACAUGA | UCAUGUCAUUCUUGCCCAG | [2285-2303] |
| 177 | AUCCAGUUCCACCAGCUUA | UAAGCUGGUGGAACUGGAU | [1178-1196] |
| 178 | CAUGAAGCUCCAUAAGGUG | CACCUUAUGGAGCUUCAUG | [2131-2149] |
| 179 | CAGGUGUAUGACAUCGAUG | CAUCGAUGUCAUACACCUG | [554-572] |
| 180 | GAGUCAAAGGUCGUCCAAG | CUUGGACGACCUUUGACUC | [1876-1894] |
| 181 | AUGCAGUCAUGUUGUAUCU | AGAUACAACAUGACUGCAU | [222-240] |
| 182 | UUUGAGGUGUUCUAUUGGA | UCCAAUAGAACACCUCAAA | [1430-1448] |
| 183 | GACAAACAGGCUGUAUGAG | CUCAUACAGCCUGUUUGUC | [1789-1807] |
| 184 | AACAGAGACCAGCGGUCUU | AAGACCGCUGGUCUCUGUU | [2165-2183] |
| 185 | UCUGAGAUACUUUUGGAGA | UCUCCAAAAGUAUCUCAGA | [1898-1916] |
| 186 | GUCACUCAUCUCCUCAUCA | UGAUGAGGAGAUGAGUGAC | [1619-1637] |
| 187 | AAGUCCUACCACUGGACCA | UGGUCCAGUGGUAGGACUU | [1155-1173] |
| 188 | GAAAAGAAAGCAUACUUGC | GCAAGUAUGCUUUCUUUUC | [2071-2089] |
| 189 | CAAGGAGAAGAAAGACUCC | GGAGUCUUUCUUCUCCUUG | [2344-2362] |
| 190 | GACUCAGCAGUUUAAGACC | GGUCUUAAACUGCUGAGUC | [340-358] |
| 191 | CAUCUUUCAUGGGCCCAAC | GUUGGGCCCAUGAAAGAUG | [1483-1501] |
| 192 | AGUGGUUUGUGAGCCUGCU | AGCAGGCUCACAAACCACU | [2187-2205] |
| 193 | CUCUUCCAGGUGUAUGACA | UGUCAUACACCUGGAAGAG | [548-566] |
| 194 | UCUUUCGAGUGGUUUGUGA | UCACAAACCACUCGAAAGA | [2180-2198] |
| 195 | UCUAGCCUCACCUCUCCAA | UUGGAGAGGUGAGGCUAGA | [2541-2559]+ |
| 196 | GAACAUCCAGUUCCACCAG | CUGGUGGAACUGGAUGUUC | [1174-1192] |
| 197 | ACUGGACCAGAACAUCCAG | CUGGAUGUUCUGGUCCAGU | [1165-1183] |
| 198 | GAGAAAGAGUCAAAGGUCG | CGACCUUUGACUCUUUCUC | [1870-1888] |
| 199 | CAUGGAAGUCAACCUCCUC | GAGGAGGUUGACUUCCAUG | [1591-1609] |
| 200 | AGAUUUUUCCAAGAGAAUU | AAUUCUCUUGGAAAAAUCU | [2522-2540] |
| 201 | AGCAGUUUAAGACCAUUGC | GCAAUGGUCUUAAACUGCU | [345-363] |
| 202 | AAUUCUGUAACAUCAAGUG | CACUUGAUGUUACAGAAUU | [1923-1941] |
| 203 | AUCGAUGGGCCUUAUGGGA | UCCCAUAAGGCCCAUCGAU | [1946-1964] |
| 204 | AUUGGACUCACCUGUCCUA | UAGGACAGGUGAGUCCAAU | [1443-1461] |
| 205 | ACUAUAGACCUGGUGACUA | UAGUCACCAGGUCUAUAGU | [1656-1674] |
| 206 | AGUUUAAGACCAUUGCAGG | CCUGCAAUGGUCUUAAACU | [348-366] |
| 207 | ACUCCUGGAUCGAAGGUGU | ACACCUUCGAUCCAGGAGU | [2103-2121] |
| 208 | CAUUGUGAGAAGUUCGGCU | AGCCGAACUUCUCACAAUG | [2501-2519] |
| 209 | ACAAACAGGCUGUAUGAGU | ACUCAUACAGCCUGUUUGU | [1790-1808] |
| 210 | CUUUGCAGAGCGAUUCUUU | AAAGAAUCGCUCUGCAAAG | [427-445] |
| 211 | AGUGCUCCUGAGCAGAAAG | CUUUCUGCUCAGGAGCACU | [1733-1751] |
| 212 | GUUGUAUCUGGAAUUGGUG | CACCAAUUCCAGAUACAAC | [232-250] |
| 213 | GGAGCUGCAUAUGUACAUG | CAUGUACAUAUGCAGCUCC | [2257-2275] |
| 214 | UGAGAAGAAGGGCAAGGUG | CACCUUGCCCUUCUUCUCA | [2434-2452] |
| 215 | GUUCUUGGUCUCACUGACU | AGUCAGUGAGACCAAGAAC | [252-270] |
| 216 | AGGCUGGUAUGCAGUCAUG | CAUGACUGCAUACCAGCCU | [214-232] |
| 217 | UCUCACUGACUUCAAGAAU | AUUCUUGAAGUCAGUGAGA | [260-278] |
| 218 | CAUAAGGUGGACUUUAUCU | AGAUAAAGUCCACCUUAUG | [2141-2159] |
| 219 | AAGGAGAAGAAAGACUCCA | UGGAGUCUUUCUUCUCCUU | [2345-2363] |
| 220 | AGCCUGCAAGAAUUCAAAG | CUUUGAAUUCUUGCAGGCU | [383-401] |
| 221 | UUUCAUGGGCCCAACUUCU | AGAAGUUGGGCCCAUGAAA | [1487-1505] |
| 222 | AGCUUAUGGGCUACGUGGU | ACCACGUAGCCCAUAAGCU | [1191-1209] |
| 223 | UGGUGGGUUCUUGGUCUCA | UGAGACCAAGAACCCACCA | [246-264] |
| 224 | UUCUGCAGAGUAUCAUGUA | UACAUGAUACUCUGCAGAA | [2043-2061] |
| 225 | AAGAAAGACUCCAUCACGG | CCGUGAUGGAGUCUUUCUU | [2351-2369] |
| 226 | ACUUGUAUCUGAACAUCCC | GGGAUGUUCAGAUACAAGU | [1674-1692] |
| 227 | CAUGGAGAACCUGACCAUC | GAUGGUCAGGUUCUCCAUG | [847-865] |
| 228 | GCUCUGCAUGUGAAAGAGU | ACUCUUUCACAUGCAGAGC | [404-422] |
| 229 | CAGUGCCUCAACUUCGACU | AGUCGAAGUUGAGGCACUG | [1070-1088] |
| 230 | CAUUCUGCAGAGUAUCAUG | CAUGAUACUCUGCAGAAUG | [2041-2059] |
| 231 | CUGUAACAUCAAGUGCUAC | GUAGCACUUGAUGUUACAG | [1927-1945] |
| 232 | CUUUCAUGGGCCCAACUUC | GAAGUUGGGCCCAUGAAAG | [1486-1504] |
| 233 | UUUCCAAGAGAAUUUCUAG | CUAGAAAUUCUCUUGGAAA | [2527-2545] |
| 234 | UUUUCACUAUAGACCUGGU | ACCAGGUCUAUAGUGAAAA | [1651-1669] |
| 235 | UGAGGUGUUCUAUUGGACU | AGUCCAAUAGAACACCUCA | [1432-1450] |
| 236 | CUUCCAGGUGUAUGACAUC | GAUGUCAUACACCUGGAAG | [550-568] |
| 237 | AUCUUGUUUUUCCUGGAGA | UCUCCAGGAAAAACAAGAU | [1529-1547] |
| 238 | CAUGUUCAUCUGCUCCAGU | ACUGGAGCAGAUGAACAUG | [1387-1405] |
| 239 | AUUCAAAGCAGCUCUGCAU | AUGCAGAGCUGCUUUGAAU | [394-412] |
| 240 | AAAGAGUCCUUCUUUGCAG | CUGCAAAGAAGGACUCUUU | [416-434] |
| 241 | UUCUGUAACAUCAAGUGCU | AGCACUUGAUGUUACAGAA | [1925-1943] |
| 242 | GGUCACUCAUCUCCUCAUC | GAUGAGGAGAUGAGUGACC | [1618-1636] |
| 243 | UGACUCAGCAGUUUAAGAC | GUCUUAAACUGCUGAGUCA | [339-357] |
| 244 | UUCACUAUAGACCUGGUGA | UCACCAGGUCUAUAGUGAA | [1653-1671] |
| 245 | AUGAGAAAGAGUCAAAGGU | ACCUUUGACUCUUUCUCAU | [1868-1886] |
| 246 | CUCCUGAGCAGAAAGACAC | GUGUCUUUCUGCUCAGGAG | [1737-1755] |
| 247 | UGGAAUUGGUGGGUUCUUG | CAAGAACCCACCAAUUCCA | [240-258] |
| 248 | UCUAAGAGGCUGUCGAGGA | UCCUCGACAGCCUCUUAGA | [1841-1859] |
| 249 | UUCGUCUGUGCCGGCUUAU | AUAAGCCGGCACAGACGAA | [171-189] |
| 250 | AGAAAGAGUCAAAGGUCGU | ACGACCUUUGACUCUUUCU | [1871-1889] |
| 251 | CAGAGCGAUUCUUUGCCCU | AGGGCAAAGAAUCGCUCUG | [432-450] |
| 252 | CUGCUCCUCCUCAUGUUCA | UGAACAUGAGGAGGAGCAG | [1376-1394] |
| 253 | GUUUGUGAGCCUGCUGACU | AGUCAGCAGGCUCACAAAC | [2191-2209] |
| 254 | UUUUUUUCACUAUAGACCU | AGGUCUAUAGUGAAAAAAA | [1648-1666] |
| 255 | AUCAUGUACAGGCACCAGA | UCUGGUGCCUGUACAUGAU | [2054-2072] |
| 256 | CUUCCUGGAGCUGCAUAUG | CAUAUGCAGCUCCAGGAAG | [2251-2269] |
| 257 | GAUCAGCCUGCAAGAAUUC | GAAUUCUUGCAGGCUGAUC | [379-397] |
| 258 | UUUGUGAGCCUGCUGACUA | UAGUCAGCAGGCUCACAAA | [2192-2210] |
| 259 | AUUCUUUGCCCUAUUUGAC | GUCAAAUAGGGCAAAGAAU | [439-457] |
| 260 | ACUUUGAGGUGUUCUAUUG | CAAUAGAACACCUCAAAGU | [1428-1446] |
| 261 | GAGAAUUUCUAGCCUCACC | GGUGAGGCUAGAAAUUCUC | [2534-2552]+ |
| 262 | AUGACAUCUGCACUGGGCA | UGCCCAGUGCAGAUGUCAU | [2273-2291] |
| 263 | CUCAACUUCGACUGCAGCU | AGCUGCAGUCGAAGUUGAG | [1076-1094] |
| 264 | UAUUUGACUCCGAUAGAAG | CUUCUAUCGGAGUCAAAUA | [450-468] |
| 265 | CUUCGACUGCAGCUUCAUC | GAUGAAGCUGCAGUCGAAG | [1081-1099] |
| 266 | CAGUUCCACCAGCUUAUGG | CCAUAAGCUGGUGGAACUG | [1181-1199] |
| 267 | UAUGAAGCCCUUAGUCCUU | AAGGACUAAGGGCUUCAUA | [129-147] |
| 268 | UCUGCACUGGGCAAGAAUG | CAUUCUUGCCCAGUGCAGA | [2279-2297] |
| 269 | GUGAGCCUGCUGACUAAAC | GUUUAGUCAGCAGGCUCAC | [2195-2213] |
| 270 | GUCCAAGACAACAUGAAGC | GCUUCAUGUUGUCUUGGAC | [2120-2138] |
| 271 | GCUCACACUGUGAACUUUG | CAAAGUUCACAGUGUGAGC | [1238-1256] |
| 272 | CAUGUUGUAUCUGGAAUUG | CAAUUCCAGAUACAACAUG | [229-247] |
| 273 | GAGUGUGACAAUGAGAAAG | CUUUCUCAUUGUCACACUC | [1858-1876] |
| 274 | ACUCAAAUUCCUCUUCCAG | CUGGAAGAGGAAUUUGAGU | [538-556] |
| 275 | CAUUGCUCGCUAUGAGUGG | CCACUCAUAGCGAGCAAUG | [1696-1714] |
| 276 | UCCAUUCUGCAGAGUAUCA | UGAUACUCUGCAGAAUGGA | [2039-2057] |
| 277 | CUUCACCAUCAGCAGUGCU | AGCACUGCUGAUGGUGAAG | [1720-1738] |
| 278 | UAUGUACAUGACAUCUGCA | UGCAGAUGUCAUGUACAUA | [2266-2284] |
| 279 | AAGGUGCAGGUCUUCUUCU | AGAAGAAGACCUGCACCUU | [2447-2465] |
| 280 | ACUGGGCAAGAAUGACAUG | CAUGUCAUUCUUGCCCAGU | [2284-2302] |
| 281 | UGCUGAGAAGAAGGGCAAG | CUUGCCCUUCUUCUCAGCA | [2431-2449] |
| 282 | UUCUUUGCCCUAUUUGACU | AGUCAAAUAGGGCAAAGAA | [440-458] |
| 283 | GUCAUGGAGAACCUGACCA | UGGUCAGGUUCUCCAUGAC | [845-863] |
| 284 | GGCAAGAAUGACAUGAAGG | CCUUCAUGUCAUUCUUGCC | [2288-2306] |
| 285 | ACUGACCCUGCUCAUCCAU | AUGGAUGAGCAGGGUCAGU | [502-520] |
| 286 | AAGGUCACUCAUCUCCUCA | UGAGGAGAUGAGUGACCUU | [1616-1634] |
| 287 | UGUGACAAUGAGAAAGAGU | ACUCUUUCUCAUUGUCACA | [1861-1879] |
| 288 | CUGCAUGUGAAAGAGUCCU | AGGACUCUUUCACAUGCAG | [407-425] |
| 289 | UGUUCUAUUGGACUCACCU | AGGUGAGUCCAAUAGAACA | [1437-1455] |
| 290 | UGCUCCUGAGCAGAAAGAC | GUCUUUCUGCUCAGGAGCA | [1735-1753] |
| 291 | CAACUUCGACUGCAGCUUC | GAAGCUGCAGUCGAAGUUG | [1078-1096] |
| 292 | CAUUGCAGGAGAAGAUGGG | CCCAUCUUCUCCUGCAAUG | [358-376] |
| 293 | AGAAAGACUCCAUCACGGG | CCCGUGAUGGAGUCUUUCU | [2352-2370] |
| 294 | UGCACUGGGCAAGAAUGAC | GUCAUUCUUGCCCAGUGCA | [2281-2299] |
| 295 | AGGUCUCAAUCCAUUGCGC | GCGCAAUGGAUUGAGACCU | [193-211] |
| 296 | CUGGACCAGAACAUCCAGU | ACUGGAUGUUCUGGUCCAG | [1166-1184] |
| 297 | UGAGAAAGAGUCAAAGGUC | GACCUUUGACUCUUUCUCA | [1869-1887] |
| 298 | AGAGAAUUUCUAGCCUCAC | GUGAGGCUAGAAAUUCUCU | [2533-2551]+ |
| 299 | CUCCAAGGUCACUCAUCUC | GAGAUGAGUGACCUUGGAG | [1612-1630] |
| 300 | CUUUCGAGUGGUUUGUGAG | CUCACAAACCACUCGAAAG | [2181-2199] |
| 301 | AUUGGUGGGUUCUUGGUCU | AGACCAAGAACCCACCAAU | [244-262] |
| 302 | AUUCCUCUUCCAGGUGUAU | AUACACCUGGAAGAGGAAU | [544-562] |
| 303 | CCUCUUCCAGGUGUAUGAC | GUCAUACACCUGGAAGAGG | [547-565] |
| 304 | UGAACUUUGUACUCCAGGC | GCCUGGAGUACAAAGUUCA | [1248-1266] |
| 305 | UUCCUGGAGCUGCAUAUGU | ACAUAUGCAGCUCCAGGAA | [2252-2270] |
| 306 | CAUGGACAAACUCAAAUUC | GAAUUUGAGUUUGUCCAUG | [529-547] |
| 307 | UCAAGGCAUCAGACCCACU | AGUGGGUCUGAUGCCUUGA | [1812-1830] |
| 308 | UUCUAGUUGCGCUUUUGCU | AGCAAAAGCGCAACUAGAA | [146-164] |
| 309 | UGUACAUGACAUCUGCACU | AGUGCAGAUGUCAUGUACA | [2268-2286] |
| 310 | AGUUCCACCAGCUUAUGGG | CCCAUAAGCUGGUGGAACU | [1182-1200] |
| 311 | AACAAGGAGAAGAAAGACU | AGUCUUUCUUCUCCUUGUU | [2342-2360] |
| 312 | GAGUAUCAUGUACAGGCAC | GUGCCUGUACAUGAUACUC | [2050-2068] |
| 313 | UCUUUCAUGGGCCCAACUU | AAGUUGGGCCCAUGAAAGA | [1485-1503] |
| 314 | CCUUAGUCCUUCUAGUUGC | GCAACUAGAAGGACUAAGG | [137-155] |
| 315 | UGUUUUUCCUGGAGAAGGC | GCCUUCUCCAGGAAAAACA | [1533-1551] |
| 316 | UAAGGUGGACUUUAUCUGG | CCAGAUAAAGUCCACCUUA | [2143-2161] |
| 317 | CUUGUUUUUCCUGGAGAAG | CUUCUCCAGGAAAAACAAG | [1531-1549] |
| 318 | AGGCACCAGAAAAGAAAGC | GCUUUCUUUUCUGGUGCCU | [2063-2081] |
| 319 | AAGCCCUUAGUCCUUCUAG | CUAGAAGGACUAAGGGCUU | [133-151] |
| 320 | UUCUAGCCUCACCUCUCCA | UGGAGAGGUGAGGCUAGAA | [2540-2558]+ |
| 321 | CUCAAAUUCCUCUUCCAGG | CCUGGAAGAGGAAUUUGAG | [539-557] |
| 322 | CAAGUGCUACAUCGAUGGG | CCCAUCGAUGUAGCACUUG | [1936-1954] |
| 323 | AAUCUUGUUUUUCCUGGAG | CUCCAGGAAAAACAAGAUU | [1528-1546] |
| 324 | CCAUUGCAGGAGAAGAUGG | CCAUCUUCUCCUGCAAUGG | [357-375] |
| 325 | UAUAGACCUGGUGACUACU | AGUAGUCACCAGGUCUAUA | [1658-1676] |
| 326 | CUACUUGUAUCUGAACAUC | GAUGUUCAGAUACAAGUAG | [1672-1690] |
| 327 | CUUUGAGGUGUUCUAUUGG | CCAAUAGAACACCUCAAAG | [1429-1447] |
| 328 | UAUCUGGAUCAACAGAGAC | GUCUCUGUUGAUCCAGAUA | [2155-2173] |
| 329 | GAAUUUCUAGCCUCACCUC | GAGGUGAGGCUAGAAAUUC | [2536-2554]+ |
| 330 | UCUGCUCCAGUUCCUGCAU | AUGCAGGAACUGGAGCAGA | [1395-1413] |
| 331 | GUUCGGCUUCAGAUUUUUC | GAAAAAUCUGAAGCCGAAC | [2512-2530] |
| 332 | UUUGCUUCCAUUCUGCAGA | UCUGCAGAAUGGAAGCAAA | [2033-2051] |
| 333 | AUCUUUCAUGGGCCCAACU | AGUUGGGCCCAUGAAAGAU | [1484-1502] |
| 334 | AUCUGCUCCAGUUCCUGCA | UGCAGGAACUGGAGCAGAU | [1394-1412] |
| 335 | UCUGGAAUUGGUGGGUUCU | AGAACCCACCAAUUCCAGA | [238-256] |
| 336 | CUGACUAAACUGGAGAUGG | CCAUCUCCAGUUUAGUCAG | [2204-2222] |
| 337 | CUGACUUCAAGAAUGAAGC | GCUUCAUUCUUGAAGUCAG | [265-283] |
| 338 | AAUGAGAAAGAGUCAAAGG | CCUUUGACUCUUUCUCAUU | [1867-1885] |
| 339 | AUCAGCCUGCAAGAAUUCA | UGAAUUCUUGCAGGCUGAU | [380-398] |
| 340 | UUGCUUCCAUUCUGCAGAG | CUCUGCAGAAUGGAAGCAA | [2034-2052] |
| 341 | AAGAAUUCAAAGCAGCUCU | AGAGCUGCUUUGAAUUCUU | [390-408] |
| 342 | UAGACCUGGUGACUACUUG | CAAGUAGUCACCAGGUCUA | [1660-1678] |
| 343 | UUAUGGGCUACGUGGUAGU | ACUACCACGUAGCCCAUAA | [1194-1212] |
| 344 | UGCAGGUCUUCUUCUGUGG | CCACAGAAGAAGACCUGCA | [2451-2469] |
| 345 | UUGAGGUGUUCUAUUGGAC | GUCCAAUAGAACACCUCAA | [1431-1449] |
| 346 | UCUGCAGAGUAUCAUGUAC | GUACAUGAUACUCUGCAGA | [2044-2062] |
| 347 | CUUAGUCCUUCUAGUUGCG | CGCAACUAGAAGGACUAAG | [138-156] |
| 348 | UCUCAAUCCAUUGCGCCCA | UGGGCGCAAUGGAUUGAGA | [196-214] |
| 349 | UUUGCAGAGCGAUUCUUUG | CAAAGAAUCGCUCUGCAAA | [428-446] |
| 350 | UAGUUGCGCUUUUGCUAUG | CAUAGCAAAAGCGCAACUA | [149-167] |
| 351 | CUUCUAGUUGCGCUUUUGC | GCAAAAGCGCAACUAGAAG | [145-163] |
| 352 | UGGGUUCUUGGUCUCACUG | CAGUGAGACCAAGAACCCA | [249-267] |
| 353 | AAGGUGUUCCAGAAAGUGG | CCACUUUCUGGAACACCUU | [2411-2429] |
| 354 | AGUCAUGGAGAACCUGACC | GGUCAGGUUCUCCAUGACU | [844-862] |
| 355 | UCAAGUGCUACAUCGAUGG | CCAUCGAUGUAGCACUUGA | [1935-1953] |
| 356 | CAUCAUGGAAGUCAACCUC | GAGGUUGACUUCCAUGAUG | [1588-1606] |
| 357 | AACAGGCUGUAUGAGUCCU | AGGACUCAUACAGCCUGUU | [1793-1811] |
| 358 | CUCAUCUCCUCAUCAAGCG | CGCUUGAUGAGGAGAUGAG | [1623-1641] |
| 359 | GUGAACUUUGUACUCCAGG | CCUGGAGUACAAAGUUCAC | [1247-1265] |
| 360 | AAAAGAAAGCAUACUUGCC | GGCAAGUAUGCUUUCUUUU | [2072-2090] |
| 361 | AUAUGUACAUGACAUCUGC | GCAGAUGUCAUGUACAUAU | [2265-2283] |
| 362 | CUUUGCUUCCAUUCUGCAG | CUGCAGAAUGGAAGCAAAG | [2032-2050] |
| 363 | UGCAAGAAUUCAAAGCAGC | GCUGCUUUGAAUUCUUGCA | [387-405] |
| 364 | GAGGUCUCAAUCCAUUGCG | CGCAAUGGAUUGAGACCUC | [192-210] |
| 365 | AAGACCAUUGCAGGAGAAG | CUUCUCCUGCAAUGGUCUU | [353-371] |
| 366 | UAGUCCUUCUAGUUGCGCU | AGCGCAACUAGAAGGACUA | [140-158] |
| 367 | ACUGACUUCAAGAAUGAAG | CUUCAUUCUUGAAGUCAGU | [264-282] |
| 368 | CUUUUUUUCACUAUAGACC | GGUCUAUAGUGAAAAAAAG | [1647-1665] |
| 369 | GUGUUCUAUUGGACUCACC | GGUGAGUCCAAUAGAACAC | [1436-1454] |
| 370 | AUAGACCUGGUGACUACUU | AAGUAGUCACCAGGUCUAU | [1659-1677] |
| 371 | UAACAUCAAGUGCUACAUC | GAUGUAGCACUUGAUGUUA | [1930-1948] |
| 372 | AGUCCUUCUUUGCAGAGCG | CGCUCUGCAAAGAAGGACU | [420-438] |
| 373 | UCUUGGUCUCACUGACUUC | GAAGUCAGUGAGACCAAGA | [254-272] |
| 374 | UUCUUUGCAGAGCGAUUCU | AGAAUCGCUCUGCAAAGAA | [425-443] |
| 375 | CAGAACAUCCAGUUCCACC | GGUGGAACUGGAUGUUCUG | [1172-1190] |
| 376 | CAGAGUAUCAUGUACAGGC | GCCUGUACAUGAUACUCUG | [2048-2066] |
| 377 | AACAUCAAGUGCUACAUCG | CGAUGUAGCACUUGAUGUU | [1931-1949] |
| 378 | ACCACUGGACCAGAACAUC | GAUGUUCUGGUCCAGUGGU | [1162-1180] |
| 379 | AGUGCCUCAACUUCGACUG | CAGUCGAAGUUGAGGCACU | [1071-1089] |
| 380 | UGGAAUCUUGUUUUUCCUG | CAGGAAAAACAAGAUUCCA | [1525-1543] |
| 381 | GAAAGAGUCAAAGGUCGUC | GACGACCUUUGACUCUUUC | [1872-1890] |
| 382 | CUUUCCAGUUCUGGGAGCU | AGCUCCCAGAACUGGAAAG | [1284-1302] |
| 383 | CAAGAAUGACAUGAAGGCC | GGCCUUCAUGUCAUUCUUG | [2290-2308] |
| 384 | UGAAAGAGUCCUUCUUUGC | GCAAAGAAGGACUCUUUCA | [414-432] |
| 385 | ACCAUUGCAGGAGAAGAUG | CAUCUUCUCCUGCAAUGGU | [356-374] |
| 386 | UCAAAGCAGCUCUGCAUGU | ACAUGCAGAGCUGCUUUGA | [396-414] |
| 387 | ACAUGACAUCUGCACUGGG | CCCAGUGCAGAUGUCAUGU | [2271-2289] |
| 388 | UGGUGACUACUUGUAUCUG | CAGAUACAAGUAGUCACCA | [1666-1684] |
| 389 | CCAUUGUGAGAAGUUCGGC | GCCGAACUUCUCACAAUGG | [2500-2518] |
| 390 | GUUCUAUUGGACUCACCUG | CAGGUGAGUCCAAUAGAAC | [1438-1456] |
| 391 | UCUGCAUGUGAAAGAGUCC | GGACUCUUUCACAUGCAGA | [406-424] |
| 392 | AUCAAGUGCUACAUCGAUG | CAUCGAUGUAGCACUUGAU | [1934-1952] |
| 393 | CGUGUGCAUCAUGGAAGUC | GACUUCCAUGAUGCACACG | [1582-1600] |
| 394 | UUUUUUCACUAUAGACCUG | CAGGUCUAUAGUGAAAAAA | [1649-1667] |
| 395 | UUUUUCCAAGAGAAUUUCU | AGAAAUUCUCUUGGAAAAA | [2525-2543] |
| 396 | ACAUCGAUGGGCCUUAUGG | CCAUAAGGCCCAUCGAUGU | [1944-1962] |
| 397 | CCUAUGAAGCCCUUAGUCC | GGACUAAGGGCUUCAUAGG | [127-145] |
| 398 | AGAGUCCUUCUUUGCAGAG | CUCUGCAAAGAAGGACUCU | [418-436] |
| 399 | GCUCCUCCUCAUGUUCAUC | GAUGAACAUGAGGAGGAGC | [1378-1396] |
| 400 | UGGAUCGAAGGUGUCCAAG | CUUGGACACCUUCGAUCCA | [2108-2126] |
| 401 | UAUUGGACUCACCUGUCCU | AGGACAGGUGAGUCCAAUA | [1442-1460] |
| 402 | UCCUUCUUUGCAGAGCGAU | AUCGCUCUGCAAAGAAGGA | [422-440] |
| 403 | UCCAAGGGCUCUGAGAUAC | GUAUCUCAGAGCCCUUGGA | [1889-1907] |
| 404 | AACAUGAAGCUCCAUAAGG | CCUUAUGGAGCUUCAUGUU | [2129-2147] |
| 405 | UAUCUGGAAUUGGUGGGUU | AACCCACCAAUUCCAGAUA | [236-254] |
| 406 | ACUCAUCUCCUCAUCAAGC | GCUUGAUGAGGAGAUGAGU | [1622-1640] |
| 407 | AAUUUCUAGCCUCACCUCU | AGAGGUGAGGCUAGAAAUU | [2537-2555]+ |
| 408 | AAACAGGCUGUAUGAGUCC | GGACUCAUACAGCCUGUUU | [1792-1810] |
| 409 | AUCUGGAAUUGGUGGGUUC | GAACCCACCAAUUCCAGAU | [237-255] |
| 410 | UAUGCAGUCAUGUUGUAUC | GAUACAACAUGACUGCAUA | [221-239] |
| 411 | CAUAUGUACAUGACAUCUG | CAGAUGUCAUGUACAUAUG | [2264-2282] |
| 412 | UUCAUCUGCUCCAGUUCCU | AGGAACUGGAGCAGAUGAA | [1391-1409] |
| 413 | AUCAACAGAGACCAGCGGU | ACCGCUGGUCUCUGUUGAU | [2162-2180] |
| 414 | UAAACUGGAGAUGGACCAG | CUGGUCCAUCUCCAGUUUA | [2209-2227] |
| 415 | AUAAGGUGGACUUUAUCUG | CAGAUAAAGUCCACCUUAU | [2142-2160] |
| 416 | CAAGACAACAUGAAGCUCC | GGAGCUUCAUGUUGUCUUG | [2123-2141] |
| 417 | UGUUCAUCUGCUCCAGUUC | GAACUGGAGCAGAUGAACA | [1389-1407] |
| 418 | UUCAUGGGCCCAACUUCUG | CAGAAGUUGGGCCCAUGAA | [1488-1506] |
| 419 | AAAUUCCUCUUCCAGGUGU | ACACCUGGAAGAGGAAUUU | [542-560] |
| 420 | CAAAUUCCUCUUCCAGGUG | CACCUGGAAGAGGAAUUUG | [541-559] |
| 421 | CCUCCUCAUGUUCAUCUGC | GCAGAUGAACAUGAGGAGG | [1381-1399] |
| 422 | UAUCAUGUACAGGCACCAG | CUGGUGCCUGUACAUGAUA | [2053-2071] |
| 423 | CAAACUCAAAUUCCUCUUC | GAAGAGGAAUUUGAGUUUG | [535-553] |
| 424 | AACUUUGUACUCCAGGCUC | GAGCCUGGAGUACAAAGUU | [1250-1268] |
| 425 | GCAGAGUAUCAUGUACAGG | CCUGUACAUGAUACUCUGC | [2047-2065] |
| 426 | UAAGAGGCUGUCGAGGAGU | ACUCCUCGACAGCCUCUUA | [1843-1861] |
| 427 | AUUGUGAGAAGUUCGGCUU | AAGCCGAACUUCUCACAAU | [2502-2520] |
| 428 | UUUAUCUGGAUCAACAGAG | CUCUGUUGAUCCAGAUAAA | [2153-2171] |
| 429 | UGGAUCAACAGAGACCAGC | GCUGGUCUCUGUUGAUCCA | [2159-2177] |
| 430 | AUUCUGUAACAUCAAGUGC | GCACUUGAUGUUACAGAAU | [1924-1942] |
| 431 | UUCAAGGCAUCAGACCCAC | GUGGGUCUGAUGCCUUGAA | [1811-1829] |
| 432 | UGCUCAUCUUUCAUGGGCC | GGCCCAUGAAAGAUGAGCA | [1479-1497] |
| 433 | UUCUGCUCAUCUUUCAUGG | CCAUGAAAGAUGAGCAGAA | [1476-1494] |
| 434 | UGACUUCAAGAAUGAAGCC | GGCUUCAUUCUUGAAGUCA | [266-284] |
| 435 | UGCAGUCAUGUUGUAUCUG | CAGAUACAACAUGACUGCA | [223-241] |
| 436 | CUAUUGGACUCACCUGUCC | GGACAGGUGAGUCCAAUAG | [1441-1459] |
| 437 | GUCUUUCGAGUGGUUUGUG | CACAAACCACUCGAAAGAC | [2179-2197] |
| 438 | UUGGUGGGUUCUUGGUCUC | GAGACCAAGAACCCACCAA | [245-263] |
| 439 | CUCAUCUUUCAUGGGCCCA | UGGGCCCAUGAAAGAUGAG | [1481-1499] |
| 440 | UCUUUGCCCUAUUUGACUC | GAGUCAAAUAGGGCAAAGA | [441-459] |
| 441 | UUCUAUUGGACUCACCUGU | ACAGGUGAGUCCAAUAGAA | [1439-1457] |
| 442 | UUUGACUCCGAUAGAAGUG | CACUUCUAUCGGAGUCAAA | [452-470] |
| 443 | UCUGCUCAUCUUUCAUGGG | CCCAUGAAAGAUGAGCAGA | [1477-1495] |
| 444 | AAAGAAAGCAUACUUGCCC | GGGCAAGUAUGCUUUCUUU | [2073-2091] |
| 445 | AGCCUGCUGACUAAACUGG | CCAGUUUAGUCAGCAGGCU | [2198-2216] |
| 446 | AAACUCAAAUUCCUCUUCC | GGAAGAGGAAUUUGAGUUU | [536-554] |
| 447 | AUCUGGAUCAACAGAGACC | GGUCUCUGUUGAUCCAGAU | [2156-2174] |
| 448 | CUUAUCCGAGGUCUCAAUC | GAUUGAGACCUCGGAUAAG | [185-203] |
| 449 | AUGUUGUAUCUGGAAUUGG | CCAAUUCCAGAUACAACAU | [230-248] |
| 450 | AUUUCUAGCCUCACCUCUC | GAGAGGUGAGGCUAGAAAU | [2538-2556]+ |
| 451 | CUUAUGGGCUACGUGGUAG | CUACCACGUAGCCCAUAAG | [1193-1211] |
| 452 | UUUCUAGCCUCACCUCUCC | GGAGAGGUGAGGCUAGAAA | [2539-2557]+ |
| 453 | GACCAGAACAUCCAGUUCC | GGAACUGGAUGUUCUGGUC | [1169-1187] |
| 454 | UUGUAUCUGGAAUUGGUGG | CCACCAAUUCCAGAUACAA | [233-251] |
| 455 | UUCGGCUUCAGAUUUUUCC | GGAAAAAUCUGAAGCCGAA | [2513-2531] |
| 456 | UACUUGUAUCUGAACAUCC | GGAUGUUCAGAUACAAGUA | [1673-1691] |
| 457 | CUUCUGCUCAUCUUUCAUG | CAUGAAAGAUGAGCAGAAG | [1475-1493] |
| 458 | AAUUGGUGGGUUCUUGGUC | GACCAAGAACCCACCAAUU | [243-261] |
| 459 | UUCCAAGAGAAUUUCUAGC | GCUAGAAAUUCUCUUGGAA | [2528-2546]+ |
| 460 | CUUCUUUGCAGAGCGAUUC | GAAUCGCUCUGCAAAGAAG | [424-442] |
| 461 | UUGUGAGAAGUUCGGCUUC | GAAGCCGAACUUCUCACAA | [2503-2521] |
| 462 | ACUAAACUGGAGAUGGACC | GGUCCAUCUCCAGUUUAGU | [2207-2225] |
| 463 | UACAUCGAUGGGCCUUAUG | CAUAAGGCCCAUCGAUGUA | [1943-1961] |
| 464 | GAGUCCUUCUUUGCAGAGC | GCUCUGCAAAGAAGGACUC | [419-437] |
| 465 | UUUGUACUCCAGGCUCAGG | CCUGAGCCUGGAGUACAAA | [1253-1271] |
| 466 | AUGGACAAACUCAAAUUCC | GGAAUUUGAGUUUGUCCAU | [530-548] |
| 467 | AUUCUGCAGAGUAUCAUGU | ACAUGAUACUCUGCAGAAU | [2042-2060] |
| 468 | UUUCACUAUAGACCUGGUG | CACCAGGUCUAUAGUGAAA | [1652-1670] |
| 469 | UGACUGGAGCAAGGUGUUC | GAACACCUUGCUCCAGUCA | [2401-2419] |
| 470 | CUGCUCAUCUUUCAUGGGC | GCCCAUGAAAGAUGAGCAG | [1478-1496] |
| 471 | UGCAGAGCGAUUCUUUGCC | GGCAAAGAAUCGCUCUGCA | [430-448] |
| 472 | GUUCAUCUGCUCCAGUUCC | GGAACUGGAGCAGAUGAAC | [1390-1408] |
| 473 | CUUUGCCCUAUUUGACUCC | GGAGUCAAAUAGGGCAAAG | [442-460] |
| 474 | UUCGACUGCAGCUUCAUCG | CGAUGAAGCUGCAGUCGAA | [1082-1100] |
| 475 | AGUUGCGCUUUUGCUAUGG | CCAUAGCAAAAGCGCAACU | [150-168] |
| 476 | UCCUUCAAGGCAUCAGACC | GGUCUGAUGCCUUGAAGGA | [1808-1826] |
| 477 | UCAUCUGCUCCAGUUCCUG | CAGGAACUGGAGCAGAUGA | [1392-1410] |
| 478 | UUGCAGAGCGAUUCUUUGC | GCAAAGAAUCGCUCUGCAA | [429-447] |
| 479 | UGCUACAUCGAUGGGCCUU | AAGGCCCAUCGAUGUAGCA | [1940-1958] |
| 480 | AACAUCCAGUUCCACCAGC | GCUGGUGGAACUGGAUGUU | [1175-1193] |
| 481 | UCAUCUCCUCAUCAAGCGG | CCGCUUGAUGAGGAGAUGA | [1624-1642] |
| 482 | GAAUUCAAAGCAGCUCUGC | GCAGAGCUGCUUUGAAUUC | [392-410] |
| 483 | UGGUUCUAAGAGGCUGUCG | CGACAGCCUCUUAGAACCA | [1837-1855] |
| 484 | AAGAAUGAAGCCGCAGACC | GGUCUGCGGCUUCAUUCUU | [273-291] |
| 485 | AUGGAAGUCAACCUCCUCC | GGAGGAGGUUGACUUCCAU | [1592-1610] |
| 486 | UCUAUUGGACUCACCUGUC | GACAGGUGAGUCCAAUAGA | [1440-1458] |
| 487 | UUCAAAGCAGCUCUGCAUG | CAUGCAGAGCUGCUUUGAA | [395-413] |
| 488 | UAUGGGCUACGUGGUAGUG | CACUACCACGUAGCCCAUA | [1195-1213] |
| 489 | GUUUUUCCUGGAGAAGGCC | GGCCUUCUCCAGGAAAAAC | [1534-1552] |
| 490 | ACUGUGAACUUUGUACUCC | GGAGUACAAAGUUCACAGU | [1244-1262] |
| 491 | UUUCGAGUGGUUUGUGAGC | GCUCACAAACCACUCGAAA | [2182-2200] |
| 492 | UUGGACUCACCUGUCCUAC | GUAGGACAGGUGAGUCCAA | [1444-1462] |
| 493 | AUUUUUCCAAGAGAAUUUC | GAAAUUCUCUUGGAAAAAU | [2524-2542] |
| 494 | CCUCAUGUUCAUCUGCUCC | GGAGCAGAUGAACAUGAGG | [1384-1402] |
| 495 | UUGUUUUUCCUGGAGAAGG | CCUUCUCCAGGAAAAACAA | [1532-1550] |
| 496 | UUCCAUUCUGCAGAGUAUC | GAUACUCUGCAGAAUGGAA | [2038-2056] |
| 497 | AUGUACAUGACAUCUGCAC | GUGCAGAUGUCAUGUACAU | [2267-2285] |
| 498 | UGUAUCUGGAAUUGGUGGG | CCCACCAAUUCCAGAUACA | [234-252] |
| 499 | AGUAUCAUGUACAGGCACC | GGUGCCUGUACAUGAUACU | [2051-2069] |
| 500 | AAACUGGAGAUGGACCAGG | CCUGGUCCAUCUCCAGUUU | [2210-2228] |

TABLE A9
DUOX2 19-mer oligomer pairs

| | | | | |
|---|---|---|---|---|
| | | | Other | Human-132566531 |
| No. | Sense siRNA | AntiSense siRNA | Sp | ORF: 204-4850 |

| | | | | |
|---|---|---|---|---|
| 1 | CCCAGACAAUUAUACAAAU | AUUUGUAUAAUUGUCUGGG | | [5002-5020] |
| 2 | ACCCAGACAAUUAUACAAA | UUUGUAUAAUUGUCUGGGU | | [5001-5019] |
| 3 | CUAAUUGACUUCCACAAAA | UUUUGUGGAAGUCAAUUAG | | [5299-5317] |
| 4 | AGAAUAUUGACCCAGACAA | UUGUCUGGGUCAAUAUUCU | | [4992-5010] |
| 5 | CAGUACUCGAUCUCAAUUC | GAAUUGAGAUCGAGUACUG | | [5699-5717] |
| 6 | UCAGUACUCGAUCUCAAUU | AAUUGAGAUCGAGUACUGA | | [5698-5716] |
| 7 | GAAUAUUGACCCAGACAAU | AUUGUCUGGGUCAAUAUUC | | [4993-5011] |
| 8 | CCACUUUCCACAUCUACUU | AAGUAGAUGUGGAAAGUGG | | [3934-3952 |
| 9 | GGGAGGAUUUUCACUUCAU | AUGAAGUGAAAAUCCUCCC | | [2965-2983 |
| 10 | GGAGCUAUUUAGGAAGGCU | AGCCUUCCUAAAUAGCUCC | | [2459-2477 |
| 11 | UGGUCCUACUAAAUAUACU | AGUAUAUUUAGUAGGACCA | | [6121-6139] |
| 12 | CUCUAAUUGACUUCCACAA | UUGUGGAAGUCAAUUAGAG | | [5297-5315] |
| 13 | GCCACGCAGUCAAUGUCUA | UAGACAUUGACUGCGUGGC | | [3634-3652 |
| 14 | CCAACACCACCCUAAACCA | UGGUUUAGGGUGGUGUUGG | | [168-186] |
| 15 | GGAGGACAACAUAGUGGUU | AACCACUAUGUUGUCCUCC | | [1406-1424 |
| 16 | CCUUUUUCAUCGGGCUCUU | AAGAGCCCGAUGAAAAAGG | | [5903-5921] |
| 17 | GGAGGUGAAAAGGUACAAU | AUUGUACCUUUUCACCUCC | | [5250-5268] |
| 18 | GUCUAAUGUUUACCAUGUA | UACAUGGUAAACAUUAGAC | | [2785-2803 |
| 19 | CCCGUCUAAUGUUUACCAU | AUGGUAAACAUUAGACGGG | | [2782-2800 |
| 20 | ACCUCAUCCUGUCCAACAA | UUGUUGGACAGGAUGAGGU | | [2284-2302 |
| 21 | UCAACCUCAUCCUGUCCAA | UUGGACAGGAUGAGGUUGA | | [2281-2299 |
| 22 | AGACAUCUGAACCCAACAC | GUGUUGGGUUCAGAUGUCU | | [156-174] |
| 23 | CAGUUUUGGUCCUACUAAA | UUUAGUAGGACCAAAACUG | | [6115-6133] |
| 24 | CCAGAUAUUCAGUACUCGA | UCGAGUACUGAAUAUCUGG | | [5690-5708] |
| 25 | CCCAAAGUGUCAGCAACUU | AAGUUGCUGACACUUUGGG | | [5518-5536] |
| 26 | UGACCAAGAAUGUAGAGAA | UUCUCUACAUUCUUGGUCA | | [4768-4786 |
| 27 | CUGUUGUCCUGGCCAUUUU | AAAAUGGCCAGGACAACAG | | [3604-3622 |
| 28 | GCCGCAACCUCAUAACCUU | AAGGUUAUGAGGUUGCGGC | | [3508-3526 |
| 29 | CCUUCAUGUUCUCUUAUAU | AUAUAAGAGAACAUGAAGG | | [3475-3493 |
| 30 | GGAGGAUUUUCACUUCAUG | CAUGAAGUGAAAAUCCUCC | | [2966-2984 |
| 31 | ACGAAUUCUUCACCAUGAU | AUCAUGGUGAAGAAUUCGU | | [2836-2854 |
| 32 | UGCCAGAUAUUCAGUACUC | GAGUACUGAAUAUCUGGCA | | [5688-5706] |
| 33 | GGCACCAGGAAACAAAGAU | AUCUUUGUUUCCUGGUGCC | | [5544-5562] |
| 34 | GAGGUGAAAAGGUACAAUA | UAUUGUACCUUUUCACCUC | | [5251-5269] |
| 35 | GGUCCCGGCAAUCAUCUAU | AUAGAUGAUUGCCGGGACC | | [3956-3974 |
| 36 | UCCUUCAUGUUCUCUUAUA | UAUAAGAGAACAUGAAGGA | | [3474-3492 |
| 37 | GAAGACAUCUGAACCCAAC | GUUGGGUUCAGAUGUCUUC | | [154-172] |
| 38 | GCAGUCCCAUCAUCAUCCA | UGGAUGAUGAUGGGACUGC | | [2179-2197 |
| 39 | CAAGAAGCUACAAAAGAAA | UUUCUUUUGUAGCUUCUUG | | [2084-2102 |
| 40 | GCCAGAUAUUCAGUACUCG | CGAGUACUGAAUAUCUGGC | | [5689-5707] |
| 41 | CAAAAUCGAUGCGUUGCUU | AAGCAACGCAUCGAUUUUG | | [5313-5331] |
| 42 | GGAGACUAUGUUCUACAAU | AUUGUAGAACAUAGUCUCC | | [5032-5050] |
| 43 | GGCAUAAAUUUGAGGUGUC | GACACCUCAAAUUUAUGCC | | [4324-4342 |
| 44 | CCGGCAAUCAUCUAUGGAG | CUCCAUAGAUGAUUGCCGG | | [3960-3978 |
| 45 | UGCCCACUUUCCACAUCUA | UAGAUGUGGAAAGUGGGCA | | [3931-3949 |
| 46 | CCAACGUCUUUGUGAAUGA | UCAUUCACAAAGACGUUGG | | [3697-3715 |
| 47 | CCAUCUGUGUUGGCGUGUU | AACACGCCAACACAGAUGG | | [3352-3370 |
| 48 | ACACCACCCUAAACCACAG | CUGUGGUUUAGGGUGGUGU | | [171-189] |
| 49 | CUAUUUAGGAAGGCUGUGA | UCACAGCCUUCCUAAAUAG | | [2463-2481 |
| 50 | GAGCUAUUUAGGAAGGCUG | CAGCCUUCCUAAAUAGCUC | | [2460-2478 |
| 51 | GAAGGAGCUAUUUAGGAAG | CUUCCUAAAUAGCUCCUUC | | [2456-2474 |
| 52 | GCGAGAAGGAGCUAUUUAG | CUAAAUAGCUCCUUCUCGC | | [2452-2470 |
| 53 | CCCUGACUGUGCUUGACUU | AAGUCAAGCACAGUCAGGG | | [1954-1972 |
| 54 | GCUACUGGUUUGAGAACAC | GUGUUCUCAAACCAGUAGC | Rat, Ms | [1750-1768 |
| 55 | CCAUUGUCCUCGACCAGUU | AACUGGUCGAGGACAAUGG | | [1708-1726 |
| 56 | GGGUCUGCAACAACUACUG | CAGUAGUUGUUGCAGACCC | Rat | [1306-1324 |
| 57 | CUGGUGUCUACAUGAGAAA | UUUCUCAUGUAGACACCAG | | [1228-1246 |
| 58 | CCUGGUGUCUACAUGAGAA | UUCUCAUGUAGACACCAGG | | [1227-1245 |
| 59 | CUGCCAGAUAUUCAGUACU | AGUACUGAAUAUCUGGCAG | | [5687-5705] |
| 60 | UCUAAUUGACUUCCACAAA | UUUGUGGAAGUCAAUUAGA | | [5298-5316] |
| 61 | CAUCUCUAAUUGACUUCCA | UGGAAGUCAAUUAGAGAUG | | [5294-5312] |
| 62 | CCAAAGAAGAUAAAGACCA | UGGUCUUUAUCUUCUUUGG | | [5154-5172] |
| 63 | CAGUGCAUGAUGAUUAUAA | UUAUAAUCAUCAUGCACUG | | [5053-5071] |
| 64 | GCAGUGCAUGAUGAUUAUA | UAUAAUCAUCAUGCACUGC | | [5052-5070] |
| 65 | CCAAGAAUGUAGAGAAGGC | GCCUUCUCUACAUUCUUGG | | [4771-4789 |
| 66 | GCAAUGGCUGUGCUGGAUA | UAUCCAGCACAGCCAUUGC | | [4258-4276 |
| 67 | CCAAGGCUUUGAGUACAAG | CUUGUACUCAAAGCCUUGG | | [4079-4097 |
| 68 | GCAAUCAUCUAUGGAGGUG | CACCUCCAUAGAUGAUUGC | | [3963-3981 |
| 69 | GUCUACAUCUUCUCAGUCA | UGACUGAGAAGAUGUAGAC | | [3648-3666 |
| 70 | AGACUUUCCUCAACCGCUA | UAGCGGUUGAGGAAAGUCU | | [3535-3553 |
| 71 | GCAACCUCAUAACCUUCCU | AGGAAGGUUAUGAGGUUGC | | [3511-3529 |
| 72 | CUAAUGUUUACCAUGUAUG | CAUACAUGGUAAACAUUAG | Rat | [2787-2805 |
| 73 | CCAUCAUCAUCCAGCUGCU | AGCAGCUGGAUGAUGAUGG | | [2185-2203 |
| 74 | AGAAGACAUCUGAACCCAA | UUGGGUUCAGAUGUCUUCU | | [153-171] |
| 75 | CUACAAAAGAAACUCAAAG | CUUUGAGUUUCUUUUGUAG | | [2091-2109 |
| 76 | GCUACAAAAGAAACUCAAA | UUUGAGUUUCUUUUGUAGC | | [2090-2108 |
| 77 | GCAACAACUACUGGAUUCG | CGAAUCCAGUAGUUGUUGC | Rat | [1312-1330 |
| 78 | UGCAACAACUACUGGAUUC | GAAUCCAGUAGUUGUUGCA | Rat | [1311-1329 |
| 79 | GGUGUCUACAUGAGAAAUG | CAUUUCUCAUGUAGACACC | | [1230-1248 |
| 80 | ACAAAAUCGAUGCGUUGCU | AGCAACGCAUCGAUUUUGU | | [5312-5330] |
| 81 | CCCACAUCUCUAAUUGACU | AGUCAAUUAGAGAUGUGGG | | [5290-5308] |
| 82 | UGCAGUGCAUGAUGAUUAU | AUAAUCAUCAUGCACUGCA | | [5051-5069] |
| 83 | CCAUCAGAAUCCACCUUAG | CUAAGGUGGAUUCUGAUGG | | [4933-4951] |
| 84 | CCCACUUCAUGCACCACUA | UAGUGGUGCAUGAAGUGGG | | [4819-4837 |
| 85 | GGCUUUGAGUACAAGUCAG | CUGACUUGUACUCAAAGCC | | [4083-4101 |
| 86 | GUCCCGGCAAUCAUCUAUG | CAUAGAUGAUUGCCGGGAC | | [3957-3975 |
| 87 | UGGCCAUCAUGUAUGUCUU | AAGACAUACAUGAUGGCCA | | [3799-3817 |
| 88 | CAACGUCUUUGUGAAUGAU | AUCAUUCACAAAGACGUUG | | [3698-3716 |
| 89 | GCUGUUGUCCUGGCCAUUU | AAAUGGCCAGGACAACAGC | | [3603-3621 |
| 90 | AGAUCGUGCUUACUACUAU | AUAGUAGUAAGCACGAUCU | | [3374-3392 |
| 91 | AGAAGAGGUUUGGCAAAAA | UUUUUGCCAAACCUCUUCU | | [3190-3208 |
| 92 | UGGAAAUGGUAUUAGAGAU | AUCUCUAAUACCAUUUCCA | | [3047-3065 |
| 93 | UGGAGGUGGAAAUGGUAUU | AAUACCAUUUCCACCUCCA | | [3041-3059 |
| 94 | GGAUUUUCACUUCAUGCUG | CAGCAUGAAGUGAAAAUCC | | [2969-2987 |
| 95 | UGACAUGGGAGGAUUUUCA | UGAAAAUCCUCCCAUGUCA | | [2959-2977 |
| 96 | UGAAGAAGGAAGCAGCCAA | UUGGCUGCUUCCUUCUUCA | | [2116-2134 |
| 97 | ACAAAAGAAACUCAAAGAG | CUCUUUGAGUUUCUUUUGU | | [2093-2111 |
| 98 | CCCUUAGUGAGUCUGCUUC | GAAGCAGACUCACUAAGGG | | [2028-2046 |
| 99 | AGCAAAGAUUUUAAGAGGA | UCCUCUUAAAAUCUUUGCU | | [133-151] |
| 100 | UCAGUGCCAUUGUCCUCGA | UCGAGGACAAUGGCACUGA | | [1702-1720 |
| 101 | CUGCAACAACUACUGGAUU | AAUCCAGUAGUUGUUGCAG | Rat | [1310-1328 |
| 102 | UCAGGGUCUGCAACAACUA | UAGUUGUUGCAGACCCUGA | Rat | [1303-1321 |
| 103 | CUCUCAGGGUCUGCAACAA | UUGUUGCAGACCCUGAGAG | Rat | [1300-1318 |
| 104 | CCAGGUCAAGACAUAGAAU | AUUCUAUGUCUUGACCUGG | | [5995-6013] |
| 105 | CCCUGAUAUUCUUGAUGUC | GACAUCAAGAAUAUCAGGG | | [5573-5591] |
| 106 | CACAUCUCUAAUUGACUUC | GAAGUCAAUUAGAGAUGUG | | [5292-5310] |
| 107 | UGGAGGUGAAAAGGUACAA | UUGUACCUUUUCACCUCCA | | [5249-5267] |
| 108 | UCCCAAAGAAGAUAAAGAC | GUCUUUAUCUUCUUUGGGA | | [5152-5170] |
| 109 | AGGAGACUAUGUUCUACAA | UUGUAGAACAUAGUCUCCU | | [5031-5049] |
| 110 | GACCCAGACAAUUAUACAA | UUGUAUAAUUGUCUGGGUC | | [5000-5018] |
| 111 | CCCGGCAAUCAUCUAUGGA | UCCAUAGAUGAUUGCCGGG | | [3959-3977 |
| 112 | GCCCACUUUCCACAUCUAC | GUAGAUGUGGAAAGUGGGC | | [3932-3950 |
| 113 | UCAUCAUCCAUGGCAGCUA | UAGCUGCCAUGGAUGAUGA | Rat, Ms | [3898-3916 |
| 114 | UCCUGGCCAUCAUGUAUGU | ACAUACAUGAUGGCCAGGA | | [3796-3814 |
| 115 | AGAAGUUCUAUUGGUGGUU | AACCACCAAUAGAACUUCU | | [3733-3751 |
| 116 | CCCAACGUCUUUGUGAAUG | CAUUCACAAAGACGUUGGG | | [3696-3714 |
| 117 | CUUAUAUCUUGCUCACCAU | AUGGUGAGCAAGAUAUAAG | | [3487-3505 |
| 118 | GUGCUUACUACUAUGGCUU | AAGCCAUAGUAGUAAGCAC | | [3379-3397 |
| 119 | AGGAUUUUCACUUCAUGCU | AGCAUGAAGUGAAAAUCCU | | [2968-2986 |
| 120 | UGGGAGGAUUUUCACUUCA | UGAAGUGAAAAUCCUCCCA | | [2964-2982 |
| 121 | CCAGAGAGAAAGGUGAGAG | CUCUCACCUUUCUCUCUGG | | [45-63] |
| 122 | GCUGUUUAGUUCUGAAGAG | CUCUUCAGAACUAAACAGC | | [2357-2375 |
| 123 | GUCAACCUCAUCCUGUCCA | UGGACAGGAUGAGGUUGAC | Rat | [2280-2298 |
| 124 | CGAGAACACAAGAAGCUAC | GUAGCUUCUUGUGUUCUCG | | [2076-2094 |
| 125 | CCAAUGUCUUUGUCUGGCA | UGCCAGACAAAGACAUUGG | | [1873-1891 |
| 126 | ACAUCCGAAAUACCACCCU | AGGGUGGUAUUUCGGAUGU | | [1804-1822 |
| 127 | GAAGCAAAGAUUUUAAGAG | CUCUUAAAAUCUUUGCUUC | | [131-149] |
| 128 | CACAGAAGCAAAGAUUUUA | UAAAAUCUUUGCUUCUGUG | | [127-145] |
| 129 | GUGAUCUCAACCCUAAUGU | ACAUUAGGGUUGAGAUCAC | | [1573-1591 |
| 130 | GGCAAAUUCUCCCGUACAG | CUGUACGGGAGAAUUUGCC | | [1455-1473 |
| 131 | ACUACUGGAUUCGGGAGAA | UUCUCCCGAAUCCAGUAGU | Rat | [1318-1336 |
| 132 | UCUGCAACAACUACUGGAU | AUCCAGUAGUUGUUGCAGA | Rat | [1309-1327 |
| 133 | AGGGUCUGCAACAACUACU | AGUAGUUGUUGCAGACCCU | Rat | [1305-1323 |
| 134 | CUCAGGGUCUGCAACAACU | AGUUGUUGCAGACCCUGAG | Rat | [1302-1320 |
| 135 | CCCUGGUGUCUACAUGAGA | UCUCAUGUAGACACCAGGG | | [1226-1244 |
| 136 | GCUGAGCUGUAAAAUAUUU | AAAUAUUUUACAGCUCAGC | | [5931-5949] |
| 137 | UCCAUACUCAGCUCUUUAA | UUAAAGAGCUGAGUAUGGA | | [5874-5892] |
| 138 | UCGAUGCGUUGCUUUGGUA | UACCAAAGCAACGCAUCGA | | [5318-5336] |
| 139 | GUGAAAAGGUACAAUAGUG | CACUAUUGUACCUUUUCAC | | [5254-5272] |
| 140 | GGUGAAAAGGUACAAUAGU | ACUAUUGUACCUUUUCACC | | [5253-5271] |
| 141 | UCCAUCAGAAUCCACCUUA | UAAGGUGGAUUCUGAUGGA | | [4932-4950] |
| 142 | CCACUUCAUGCACCACUAU | AUAGUGGUGCAUGAAGUGG | | [4820-4838 |
| 143 | GAAUGACCAAGAAUGUAGA | UCUACAUUCUUGGUCAUUC | | [4765-4783 |
| 144 | CCAAAUGCUGUGUAAGAAG | CUUCUUACACAGCAUUUGG | | [4424-4442 |
| 145 | CAAUGGCUGUGCUGGAUAC | GUAUCCAGCACAGCCAUUG | | [4259-4277 |
| 146 | CAAUCAUCUAUGGAGGUGA | UCACCUCCAUAGAUGAUUG | | [3964-3982 |
| 147 | GGCAAUCAUCUAUGGAGGU | ACCUCCAUAGAUGAUUGCC | | [3962-3980 |
| 148 | CGGCAAUCAUCUAUGGAGG | CCUCCAUAGAUGAUUGCCG | | [3961-3979 |
| 149 | CCCACUUUCCACAUCUACU | AGUAGAUGUGGAAAGUGGG | | [3933-3951 |
| 150 | UCAUAACCUUCCUGCGAGA | UCUCGCAGGAAGGUUAUGA | | [3517-3535 |
| 151 | GCUGACAUGGGAGGAUUUU | AAAAUCCUCCCAUGUCAGC | | [2957-2975 |
| 152 | CCAAGGACGAAUUCUUCAC | GUGAAGAAUUCGUCCUUGG | | [2830-2848 |
| 153 | CCGUCUAAUGUUUACCAUG | CAUGGUAAACAUUAGACGG | | [2783-2801 |
| 154 | GUUUAGUUCUGAAGAGGAA | UUCCUCUUCAGAACUAAAC | | [2360-2378 |
| 155 | GCUGCUGUUUAGUUCUGAA | UUCAGAACUAAACAGCAGC | | [2354-2372 |
| 156 | UGCUGCUCAAGAUCCCUAA | UUAGGGAUCUUGAGCAGCA | | [2320-2338 |
| 157 | UUAAGAGGAAAGAAGACAU | AUGUCUUCUUUCCUCUUAA | | [143-161] |
| 158 | GCAGCCCAAUGUCUUUGUC | GACAAAGACAUUGGGCUGC | | [1868-1886 |
| 159 | CAGAAGCAAAGAUUUUAAG | CUUAAAAUCUUUGCUUCUG | | [129-147] |
| 160 | CCUCCAUACUCAGCUCUUU | AAAGAGCUGAGUAUGGAGG | | [5872-5890] |
| 161 | CCCUCCAUACUCAGCUCUU | AAGAGCUGAGUAUGGAGGG | | [5871-5889] |
| 162 | CAGAUAUUCAGUACUCGAU | AUCGAGUACUGAAUAUCUG | | [5691-5709] |
| 163 | CAGCCCUGAUAUUCUUGAU | AUCAAGAAUAUCAGGGCUG | | [5570-5588] |
| 164 | CCAGCCCUGAUAUUCUUGA | UCAAGAAUAUCAGGGCUGG | | [5569-5587] |
| 165 | GAGGGCACCAGGAAACAAA | UUUGUUUCCUGGUGCCCUC | | [5541-5559] |
| 166 | CGAUGCGUUGCUUUGGUAU | AUACCAAAGCAACGCAUCG | | [5319-5337] |
| 167 | GUGUGGAGGUGAAAAGGUA | UACCUUUUCACCUCCACAC | | [5246-5264] |
| 168 | CAAAGAAGAUAAAGACCAC | GUGGUCUUUAUCUUCUUUG | | [5155-5173] |
| 169 | GCAUGAUGAUUAUAAGUCC | GGACUUAUAAUCAUCAUGC | | [5057-5075] |
| 170 | UGCACCACUAUGAGAACUU | AAGUUCUCAUAGUGGUGCA | | [4828-4846 |
| 171 | UGGCAUAAAUUUGAGGUGU | ACACCUCAAAUUUAUGCCA | | [4323-4341 |
| 172 | UCGCCACCUACCAGAACAU | AUGUUCUGGUAGGUGGCGA | | [1072-1090 |
| 173 | GGAGUGGCAUAAAUUUGAG | CUCAAAUUUAUGCCACUCC | | [4319-4337 |
| 174 | CUGCCCACUUUCCACAUCU | AGAUGUGGAAAGUGGGCAG | | [3930-3948 |
| 175 | UCUAUGCCCUGCUCAUCAU | AUGAUGAGCAGGGCAUAGA | | [3886-3904 |
| 176 | ACCUCUACAUCCUGCUCUA | UAGAGCAGGAUGUAGAGGU | | [3871-3889 |
| 177 | CCAUCAUGUAUGUCUUCGC | GCGAAGACAUACAUGAUGG | | [3802-3820 |
| 178 | GGCCAUCAUGUAUGUCUUC | GAAGACAUACAUGAUGGCC | | [3800-3818 |
| 179 | CUGGCCAUCAUGUAUGUCU | AGACAUACAUGAUGGCCAG | | [3798-3816 |
| 180 | GUCCUGGCCAUCAUGUAUG | CAUACAUGAUGGCCAGGAC | | [3795-3813 |
| 181 | GGUCCUGGCCAUCAUGUAU | AUACAUGAUGGCCAGGACC | | [3794-3812 |
| 182 | GAAGUUCUAUUGGUGGUUC | GAACCACCAAUAGAACUUC | | [3734-3752 |
| 183 | CCUUCCUGCGAGAGACUUU | AAAGUCUCUCGCAGGAAGG | | [3523-3541 |
| 184 | CUCAUAACCUUCCUGCGAG | CUCGCAGGAAGGUUAUGAG | | [3516-3534 |
| 185 | CGCAACCUCAUAACCUUCC | GGAAGGUUAUGAGGUUGCG | | [3510-3528 |
| 186 | CCGCAACCUCAUAACCUUC | GAAGGUUAUGAGGUUGCGG | | [3509-3527 |
| 187 | CUCCUUCAUGUUCUCUUAU | AUAAGAGAACAUGAAGGAG | | [3473-3491 |
| 188 | GCUUACUACUAUGGCUUUG | CAAAGCCAUAGUAGUAAGC | | [3381-3399 |
| 189 | UGCUUACUACUAUGGCUUU | AAAGCCAUAGUAGUAAGCA | | [3380-3398 |
| 190 | CGUGCUUACUACUAUGGCU | AGCCAUAGUAGUAAGCACG | | [3378-3396 |
| 191 | GAAGAGGUUUGGCAAAAAG | CUUUUUGCCAAACCUCUUC | | [3191-3209 |
| 192 | AGGUGGAAAUGGUAUUAGA | UCUAAUACCAUUUCCACCU | | [3044-3062 |
| 193 | GAGGUGGAAAUGGUAUUAG | CUAAUACCAUUUCCACCUC | | [3043-3061 |
| 194 | GUAUGACCUGGAUGAGAAU | AUUCUCAUCCAGGUCAUAC | | [2801-2819 |
| 195 | UCCCGUCUAAUGUUUACCA | UGGUAAACAUUAGACGGGA | | [2781-2799 |
| 196 | UGGCUGACAAGGAUGGCAA | UUGCCAUCCUUGUCAGCCA | Ms | [2692-2710 |
| 197 | AACACAAGAAGCUACAAAA | UUUUGUAGCUUCUUGUGUU | | [2080-2098 |
| 198 | AGAACACAAGAAGCUACAA | UUGUAGCUUCUUGUGUUCU | | [2078-2096 |
| 199 | GCCGAGAACACAAGAAGCU | AGCUUCUUGUGUUCUCGGC | | [2074-2092 |
| 200 | AUUGAAGACAUCCGAAAUA | UAUUUCGGAUGUCUUCAAU | | [1797-1815 |
| 201 | CCUGUUCAGUGCCAUUGUC | GACAAUGGCACUGAACAGG | | [1697-1715 |
| 202 | GGACAACAUAGUGGUUGAA | UUCAACCACUAUGUUGUCC | | [1409-1427 |
| 203 | GCAAGAAGUAGAGCACAGA | UCUGUGCUCUACUUCUUGC | | [114-132] |
| 204 | GGCAAGAAGUAGAGCACAG | CUGUGCUCUACUUCUUGCC | | [113-131] |
| 205 | CCAAUCUGAACAGUACCCA | UGGGUACUGUUCAGAUUGG | | [1339-1357 |
| 206 | GGUUUUCAAAGCUCCCAAG | CUUGGGAGCUUUGAAAACC | | [1281-1299 |
| 207 | ACAAGGGUUUUCAAAGCUC | GAGCUUUGAAAACCCUUGU | | [1276-1294 |
| 208 | AGAAAUGCCAGCUGUCAUU | AAUGACAGCUGGCAUUUCU | | [1242-1260 |
| 209 | UGGUGUCUACAUGAGAAAU | AUUUCUCAUGUAGACACCA | | [1229-1247 |
| 210 | GUCAAGACAUAGAAUGUUU | AAACAUUCUAUGUCUUGAC | | [5999-6017] |
| 211 | GGUCAAGACAUAGAAUGUU | AACAUUCUAUGUCUUGACC | | [5998-6016] |
| 212 | CCAUACUCAGCUCUUUAAC | GUUAAAGAGCUGAGUAUGG | | [5875-5893] |
| 213 | UGAGGGUGAAGGCAAGAAG | CUUCUUGCCUUCACCCUCA | | [103-121] |
| 214 | GGGCACCAGGAAACAAAGA | UCUUUGUUUCCUGGUGCCC | | [5543-5561] |
| 215 | GUGAGGGUGAAGGCAAGAA | UUCUUGCCUUCACCCUCAC | | [102-120] |
| 216 | GGACUGACAUUUGAGGGAG | CUCCCUCAAAUGUCAGUCC | | [5344-5362] |
| 217 | CAAGUGUGGAGGUGAAAAG | CUUUUCACCUCCACACUUG | | [5243-5261] |
| 218 | GACAAGUGUGGAGGUGAAA | UUUCACCUCCACACUUGUC | | [5241-5259] |
| 219 | AGAAAAGGCAGGAGACUAU | AUAGUCUCCUGCCUUUUCU | | [5022-5040] |
| 220 | CUCCAUCAGAAUCCACCUU | AAGGUGGAUUCUGAUGGAG | | [4931-4949] |
| 221 | ACAUUUAUGUCACCCAGCU | AGCUGGGUGACAUAAAUGU | | [4546-4564 |
| 222 | UGGUCUUCAAGUCAUCCUU | AAGGAUGACUUGAAGACCA | | [4399-4417 |
| 223 | CUGGAAGAGUGAGGGUGAA | UUCACCCUCACUCUUCCAG | | [94-112] |
| 224 | UCAUCUAUGGAGGUGACAA | UUGUCACCUCCAUAGAUGA | | [3967-3985 |
| 225 | UGACCCACCACCUCUACAU | AUGUAGAGGUGGUGGGUCA | | [3862-3880 |
| 226 | CCAUGUUCUUUCCGACGUG | CACGUCGGAAAGAACAUGG | | [533-551 |
| 227 | CAACCGCUAUGUGCCUUUU | AAAAGGCACAUAGCGGUUG | | [3545-3563 |
| 228 | GACUUUCCUCAACCGCUAU | AUAGCGGUUGAGGAAAGUC | | [3536-3554 |
| 229 | CAACCUCAUAACCUUCCUG | CAGGAAGGUUAUGAGGUUG | | [3512-3530 |
| 230 | UCUCCUUCAUGUUCUCUUA | UAAGAGAACAUGAAGGAGA | | [3472-3490 |
| 231 | GUCUCCUUCAUGUUCUCUU | AAGAGAACAUGAAGGAGAC | | [3471-3489 |
| 232 | CCAGCGUCUCCUUCAUGUU | AACAUGAAGGAGACGCUGG | | [3466-3484 |
| 233 | CAUGGGAGGAUUUUCACUU | AAGUGAAAAUCCUCCCAUG | | [2962-2980 |
| 234 | AGCUGACAUGGGAGGAUUU | AAAUCCUCCCAUGUCAGCU | | [2956-2974 |
| 235 | GCUAUGACGGCUGGUUUAA | UUAAACCAGCCGUCAUAGC | | [313-331 |
| 236 | GUCCCAUCAUCAUCCAGCU | AGCUGGAUGAUGAUGGGAC | | [2182-2200 |
| 237 | CAAAAGAAACUCAAAGAGA | UCUCUUUGAGUUUCUUUUG | | [2094-2112 |
| 238 | ACACAAGAAGCUACAAAAG | CUUUUGUAGCUUCUUGUGU | | [2081-2099 |
| 239 | GAGAACACAAGAAGCUACA | UGUAGCUUCUUGUGUUCUC | | [2077-2095 |
| 240 | CCGAGAACACAAGAAGCUA | UAGCUUCUUGUGUUCUCGG | | [2075-2093 |
| 241 | GACUGUGCUUGACUUCUUU | AAAGAAGUCAAGCACAGUC | | [1958-1976 |
| 242 | CAAUGUCUUUGUCUGGCAU | AUGCCAGACAAAGACAUUG | | [1874-1892 |
| 243 | CCCUGUUCAGUGCCAUUGU | ACAAUGGCACUGAACAGGG | | [1696-1714 |
| 244 | CUAAAAAGCUGAGCUGUAA | UUACAGCUCAGCUUUUUAG | Rat | [5924-5942] |
| 245 | UGCAUCUACUGGAGUGGUC | GACCACUCCAGUAGAUGCA | | [5635-5653] |
| 246 | CCUCUCUGCAUCUACUGGA | UCCAGUAGAUGCAGAGAGG | | [5629-5647] |
| 247 | UGCGUUGCUUUGGUAUUUG | CAAAUACCAAAGCAACGCA | | [5322-5340] |
| 248 | GUGGAGGUGAAAAGGUACA | UGUACCUUUUCACCUCCAC | | [5248-5266] |
| 249 | ACAAGUGUGGAGGUGAAAA | UUUUCACCUCCACACUUGU | | [5242-5260] |
| 250 | UGCAUGAUGAUUAUAAGUC | GACUUAUAAUCAUCAUGCA | | [5056-5074] |
| 251 | AGUGCAUGAUGAUUAUAAG | CUUAUAAUCAUCAUGCACU | | [5054-5072] |
| 252 | CAAAUGAGAAAAGGCAGGA | UCCUGCCUUUUCUCAUUUG | | [5016-5034] |
| 253 | UGCCUUCUCUUCUGUGCAC | GUGCACAGAAGAGAAGGCA | | [4884-4902] |
| 254 | CCACUAUGAGAACUUCUGA | UCAGAAGUUCUCAUAGUGG | | [4832-4850 |
| 255 | AGAAUGUAGAGAAGGCCUG | CAGGCCUUCUCUACAUUCU | | [4774-4792 |
| 256 | ACAUCGCUGUGUAUGAGUG | CACUCAUACACAGCGAUGU | | [1087-1105 |
| 257 | GUGGCAUAAAUUUGAGGUG | CACCUCAAAUUUAUGCCAC | | [4322-4340 |
| 258 | GAGUGACCUACCUGCAAUU | AAUUGCAGGUAGGUCACUC | | [4051-4069 |
| 259 | CCUCUACAUCCUGCUCUAU | AUAGAGCAGGAUGUAGAGG | | [3872-3890 |
| 260 | GCCAUCAUGUAUGUCUUCG | CGAAGACAUACAUGAUGGC | | [3801-3819 |
| 261 | AGGUAUGACAGGUGUGCUU | AAGCACACCUGUCAUACCU | | [3767-3785 |
| 262 | CAGAAGUUCUAUUGGUGGU | ACCACCAAUAGAACUUCUG | | [3732-3750 |
| 263 | UCUUAUAUCUUGCUCACCA | UGGUGAGCAAGAUAUAAGA | | [3486-3504 |
| 264 | CAGAUCGUGCUUACUACUA | UAGUAGUAAGCACGAUCUG | | [3373-3391 |
| 265 | GCAAUCUUCUCGGCCAUCU | AGAUGGCCGAGAAGAUUGC | | [3339-3357 |
| 266 | AGGCGCUGCAAGAGAAGAU | AUCUUCUCUUGCAGCGCCU | | [3241-3259 |
| 267 | CAGAGAGAAAGGUGAGAGU | ACUCUCACCUUUCUCUCUG | | [46-64] |
| 268 | UGUUCUCUCUGGCUGACAA | UUGUCAGCCAGAGAGAACA | | [2683-2701 |
| 269 | ACAUGUUUGUGGAGUCCAU | AUGGACUCCACAAACAUGU | Ms | [2665-2683 |
| 270 | CUGCUGUUUAGUUCUGAAG | CUUCAGAACUAAACAGCAG | | [2355-2373 |
| 271 | GAAGGCAAGAAGUAGAGCA | UGCUCUACUUCUUGCCUUC | | [110-128] |
| 272 | AGGGUGAAGGCAAGAAGUA | UACUUCUUGCCUUCACCCU | | [105-123] |
| 273 | CCUAGAAGCAACCACUGAU | AUCAGUGGUUGCUUCUAGG | | [6079-6097] |
| 274 | AAGCUGAGCUGUAAAAUAU | AUAUUUUACAGCUCAGCUU | | [5929-5947] |
| 275 | CUGAUAUUCUUGAUGUCAC | GUGACAUCAAGAAUAUCAG | | [5575-5593] |
| 276 | AGCCCUGAUAUUCUUGAUG | CAUCAAGAAUAUCAGGGCU | | [5571-5589] |
| 277 | CUGUGAUGCUUAGAACCUG | CAGGUUCUAAGCAUCACAG | | [5415-5433] |
| 278 | AUGCGUUGCUUUGGUAUUU | AAAUACCAAAGCAACGCAU | | [5321-5339] |
| 279 | CAGGAGACUAUGUUCUACA | UGUAGAACAUAGUCUCCUG | | [5030-5048] |
| 280 | UCGCUGUGUAUGAGUGGCU | AGCCACUCAUACACAGCGA | | [1090-1108 |
| 281 | UCAUGUAUGUCUUCGCCUC | GAGGCGAAGACAUACAUGA | | [3805-3823 |
| 282 | CAGUCAAUGUCUACAUCUU | AAGAUGUAGACAUUGACUG | | [3640-3658 |
| 283 | ACGGUGUGUAUCAGGCUCU | AGAGCCUGAUACACACCGU | | [403-421 |
| 284 | ACAAGCGCUUCGUGGAGAA | UUCUCCACGAAGCGCUUGU | | [3295-3313 |
| 285 | CAAGAGAAGAUGCAGCGAG | CUCGCUGCAUCUUCUCUUG | | [3249-3267 |
| 286 | GGACUGAAGAAGAGGUUUG | CAAACCUCUUCUUCAGUCC | | [3183-3201 |
| 287 | ACCUGGUGCUGCUGUUUAG | CUAAACAGCAGCACCAGGU | | [2347-2365 |
| 288 | UGACCUGGUGCUGCUGUUU | AAACAGCAGCACCAGGUCA | | [2345-2363 |
| 289 | UCACCAUCAUUGCUCUCUG | CAGAGAGCAAUGAUGGUGA | | [2002-2020 |
| 290 | GCCAUCACCAUCAUUGCUC | GAGCAAUGAUGGUGAUGGC | | [1998-2016 |
| 291 | GUCUUUGUCUGGCAUAAAG | CUUUAUGCCAGACAAAGAC | | [1878-1896 |
| 292 | AGCCCAAUGUCUUUGUCUG | CAGACAAAGACAUUGGGCU | | [1870-1888 |
| 293 | GCAAAUUCUCCCGUACAGA | UCUGUACGGGAGAAUUUGC | | [1456-1474 |
| 294 | CAAGAAGUAGAGCACAGAA | UUCUGUGCUCUACUUCUUG | | [115-133] |
| 295 | CAGGUCAAGACAUAGAAUG | CAUUCUAUGUCUUGACCUG | | [5996-6014] |
| 296 | AGCUGAGCUGUAAAAUAUU | AAUAUUUUACAGCUCAGCU | | [5930-5948] |
| 297 | CCUAAAAAGCUGAGCUGUA | UACAGCUCAGCUUUUUAGG | | [5923-5941] |
| 298 | CUCCAUACUCAGCUCUUUA | UAAAGAGCUGAGUAUGGAG | | [5873-5891] |
| 299 | UGCUUGGACUGACAUUUGA | UCAAAUGUCAGUCCAAGCA | | [5339-5357] |
| 300 | GCUUUGGUAUUUGCUUGGA | UCCAAGCAAAUACCAAAGC | | [5328-5346] |
| 301 | AAAAGGUACAAUAGUGCUU | AAGCACUAUUGUACCUUUU | | [5257-5275] |
| 302 | ACCACUAUGAGAACUUCUG | CAGAAGUUCUCAUAGUGGU | | [4831-4849 |
| 303 | CCUUCUUCAACUCCCUGCA | UGCAGGGAGUUGAAGAAGG | | [4693-4711 |
| 304 | CUGGUCUUCAAGUCAUCCU | AGGAUGACUUGAAGACCAG | | [4398-4416 |
| 305 | UGACCUACCUGCAAUUCCA | UGGAAUUGCAGGUAGGUCA | | [4054-4072 |
| 306 | UCUCUUAUAUCUUGCUCAC | GUGAGCAAGAUAUAAGAGA | | [3484-3502 |
| 307 | UCGUGUGUGUGGCAAUCUU | AAGAUUGCCACACACACGA | | [3328-3346 |
| 308 | AAGAAGAGGUUUGGCAAAA | UUUUGCCAAACCUCUUCUU | | [3189-3207 |
| 309 | GAAGAAGAGGUUUGGCAAA | UUUGCCAAACCUCUUCUUC | | [3188-3206 |
| 310 | CUGUGUCAAAGGUGGAGGU | ACCUCCACCUUUGACACAG | | [3023-3041 |
| 311 | CUGGUGGUCUUCAUGAAAG | CUUUCAUGAAGACCACCAG | Ms | [2745-2763 |
| 312 | CUGGUGCUGCUGUUUAGUU | AACUAAACAGCAGCACCAG | | [2349-2367 |
| 313 | GUUUUGCCAUCACCAUCAU | AUGAUGGUGAUGGCAAAAC | | [1993-2011 |
| 314 | UGCAGCCCAAUGUCUUUGU | ACAAAGACAUUGGGCUGCA | | [1867-1885 |
| 315 | AGAUUUCGGAGUUGGAGGA | UCCUCCAACUCCGAAAUCU | | [1393-1411 |
| 316 | CCCAAUCUGAACAGUACCC | GGGUACUGUUCAGAUUGGG | | [1338-1356 |
| 317 | CUAGAAGCAACCACUGAUG | CAUCAGUGGUUGCUUCUAG | | [6080-6098] |
| 318 | UCAAGACAUAGAAUGUUUC | GAAACAUUCUAUGUCUUGA | | [6000-6018] |
| 319 | GUGAUGCUUAGAACCUGGA | UCCAGGUUCUAAGCAUCAC | | [5417-5435] |
| 320 | CACUAUGAGAACUUCUGAG | CUCAGAAGUUCUCAUAGUG | | [4833-4851 |
| 321 | CUAUGGAGGUGACAAGCUG | CAGCUUGUCACCUCCAUAG | | [3971-3989 |
| 322 | CACCUCUACAUCCUGCUCU | AGAGCAGGAUGUAGAGGUG | | [3870-3888 |
| 323 | ACCAUGUUCUUUCCGACGU | ACGUCGGAAAGAACAUGGU | | [532-550 |
| 324 | AGAGAAAGGUGAGAGUGCA | UGCACUCUCACCUUUCUCU | | [49-67] |
| 325 | ACAUCGUGUGUGUGGCAAU | AUUGCCACACACACGAUGU | | [3325-3343 |
| 326 | GAGUCUAUGUUCCGGGAGU | ACUCCCGGAACAUAGACUC | Ms | [2916-2934 |
| 327 | GUGGUCUUCAUGAAAGGCU | AGCCUUUCAUGAAGACCAC | Ms | [2748-2766 |
| 328 | UGUUUGUGGAGUCCAUGUU | AACAUGGACUCCACAAACA | Rat, Ms | [2668-2686 |
| 329 | CAGACACCUUUUUGCUCAG | CUGAGCAAAAAGGUGUCUG | | [2519-2537 |
| 330 | UCAGACACCUUUUUGCUCA | UGAGCAAAAAGGUGUCUGA | | [2518-2536 |
| 331 | GCCUUCCCUUAGUGAGUCU | AGACUCACUAAGGGAAGGC | | [2023-2041 |
| 332 | CUGUGCUUGACUUCUUUGA | UCAAAGAAGUCAAGCACAG | | [1960-1978 |
| 333 | CUCCCGUACAGACUAUGUG | CACAUAGUCUGUACGGGAG | | [1463-1481 |
| 334 | CCAGAUUUCGGAGUUGGAG | CUCCAACUCCGAAAUCUGG | | [1391-1409 |
| 335 | GAGAAAUGCCAGCUGUCAU | AUGACAGCUGGCAUUUCUC | | [1241-1259 |
| 336 | AAAAAGCUGAGCUGUAAAA | UUUUACAGCUCAGCUUUUU | | [5926-5944] |
| 337 | CUUGAUGUCACCAGUGAUA | UAUCACUGGUGACAUCAAG | | [5583-5601] |
| 338 | CCUGAUAUUCUUGAUGUCA | UGACAUCAAGAAUAUCAGG | | [5574-5592] |
| 339 | UGGACUGACAUUUGAGGGA | UCCCUCAAAUGUCAGUCCA | | [5343-5361] |
| 340 | GUAUUUGCUUGGACUGACA | UGUCAGUCCAAGCAAAUAC | | [5334-5352] |
| 341 | AAGUCCACCUGUUUAUCAA | UUGAUAAACAGGUGGACUU | | [5070-5088] |
| 342 | AGAGUGAGGGUGAAGGCAA | UUGCCUUCACCCUCACUCU | | [99-117] |
| 343 | UGAUUAUAAGUCCACCUGU | ACAGGUGGACUUAUAAUCA | | [5063-5081] |
| 344 | CUUCUCUUCUGUGCACCUA | UAGGUGCACAGAAGAGAAG | | [4887-4905] |
| 345 | AGCCCUUCUUCAACUCCCU | AGGGAGUUGAAGAAGGGCU | | [4690-4708 |
| 346 | AGAAAGUGCUGAACCGGAG | CUCCGGUUCAGCACUUUCU | | [4621-4639 |
| 347 | CCUUUGCCUCCAUCCUCAA | UUGAGGAUGGAGGCAAAGG | Rat, Ms | [4375-4393 |
| 348 | CCUUGAUGGACCGUUUGGA | UCCAAACGGUCCAUCAAGG | | [4289-4307 |
| 349 | CUACCAUGUUCUUUCCGAC | GUCGGAAAGAACAUGGUAG | | [530-548 |
| 350 | GCUACCAUGUUCUUUCCGA | UCGGAAAGAACAUGGUAGC | | [529-547 |
| 351 | UUGUGAAUGAUGGGUCCAA | UUGGACCCAUCAUUCACAA | Rat | [3706-3724 |
| 352 | GUGUGUAUCAGGCUCUGGA | UCCAGAGCCUGAUACACAC | | [406-424 |
| 353 | GCUGUCGAGUCUCGUUCAU | AUGAACGAGACUCGACAGC | | [3085-3103 |
| 354 | UGGUCUUCAUGAAAGGCUC | GAGCCUUUCAUGAAGACCA | Ms | [2749-2767 |
| 355 | CCAGGACAUGUUUGUGGAG | CUCCACAAACAUGUCCUGG | Ms | [2660-2678 |
| 356 | UGGUGCUGCUGUUUAGUUC | GAACUAAACAGCAGCACCA | | [2350-2368 |
| 357 | UCAUUGCUCUCUGCUGCCU | AGGCAGCAGAGAGCAAUGA | | [2008-2026 |
| 358 | UGUCUUUGUCUGGCAUAAA | UUUAUGCCAGACAAAGACA | | [1877-1895 |
| 359 | AUGUCUUUGUCUGGCAUAA | UUAUGCCAGACAAAGACAU | | [1876-1894 |
| 360 | CCUGCAGCCCAAUGUCUUU | AAAGACAUUGGGCUGCAGG | | [1865-1883 |
| 361 | AGGAGGUGAAUGAGCUGCU | AGCAGCUCAUUCACCUCCU | | [1357-1375 |
| 362 | CAGCUGUCAUUUCCGGAAG | CUUCCGGAAAUGACAGCUG | | [1250-1268 |
| 363 | UCGGGCUCUUUCCUAAAAA | UUUUUAGGAAAGAGCCCGA | | [5912-5930] |
| 364 | GGUAUUUGCUUGGACUGAC | GUCAGUCCAAGCAAAUACC | | [5333-5351] |
| 365 | AAAGGCAGGAGACUAUGUU | AACAUAGUCUCCUGCCUUU | | [5025-5043] |
| 366 | GGAGAAUAUUGACCCAGAC | GUCUGGGUCAAUAUUCUCC | | [4990-5008] |
| 367 | UGAAGAAGAGGUUUGGCAA | UUGCCAAACCUCUUCUUCA | | [3187-3205 |
| 368 | CUGAAGAAGAGGUUUGGCA | UGCCAAACCUCUUCUUCAG | | [3186-3204 |
| 369 | GGAGCUCUUCUGACUGGAU | AUCCAGUCAGAAGAGCUCC | | [240-258 |
| 370 | CAGGACAUGUUUGUGGAGU | ACUCCACAAACAUGUCCUG | Ms | [2661-2679 |
| 371 | GGAAUUUGUGGUGGCCUCU | AGAGGCCACCACAAAUUCC | | [1181-1199 |
| 372 | AGGCAGGAGACUAUGUUCU | AGAACAUAGUCUCCUGCCU | | [5027-5045] |
| 373 | GAGAACUUCUGAGCCUGUC | GACAGGCUCAGAAGUUCUC | | [4839-4857 |
| 374 | UGGAGAUCAGCGUGGUGAA | UUCACCACGCUGAUCUCCA | | [4012-4030 |
| 375 | ACAUUGCACAGACCACCCU | AGGGUGGUCUGUGCAAUGU | | [3415-3433 |
| 376 | UGGAGGUGGAGGUGGAAAU | AUUUCCACCUCCACCUCCA | | [3035-3053 |
| 377 | CGGAGUUGGAGGACAACAU | AUGUUGUCCUCCAACUCCG | | [1399-1417 |
| 378 | UCCACAAAAUCGAUGCGUU | AACGCAUCGAUUUUGUGGA | | [5309-5327] |
| 379 | GGGAGAAUAUUGACCCAGA | UCUGGGUCAAUAUUCUCCC | | [4989-5007] |
| 380 | CCAGGAAUGACCAAGAAUG | CAUUCUUGGUCAUUCCUGG | | [4761-4779 |
| 381 | UCGUUCAUCACUCGGACAC | GUGUCCGAGUGAUGAACGA | | [3096-3114 |
| 382 | GGUGGAGGUGGAAAUGGUA | UACCAUUUCCACCUCCACC | | [3039-3057 |
| 383 | GCAAUGGCUACCUGUCCUU | AAGGACAGGUAGCCAUUGC | | [2707-2725 |
| 384 | AGGAAAGAAGACAUCUGAA | UUCAGAUGUCUUCUUUCCU | | [148-166] |
| 385 | CUGGAGUGGUGGCCUAUUU | AAAUAGGCCACCACUCCAG | | [2050-2068 |
| 386 | AGAGGAAAGAAGACAUCUG | CAGAUGUCUUCUUUCCUCU | | [146-164] |
| 387 | GGAACUGGAGUGAUCUCAA | UUGAGAUCACUCCAGUUCC | | [1564-1582 |
| 388 | GGAGUUGGAGGACAACAUA | UAUGUUGUCCUCCAACUCC | | [1400-1418 |
| 389 | CCAGUGCAGAAGCUGUGAU | AUCACAGCUUCUGCACUGG | | [5403-5421] |
| 390 | CCAGUCAGGAUCUCCCAAA | UUUGGGAGAUCCUGACUGG | | [5140-5158] |
| 391 | GCACAUUUAUGUCACCCAG | CUGGGUGACAUAAAUGUGC | | [4544-4562 |
| 392 | GGCAGCCAAAUGCUGUGUA | UACACAGCAUUUGGCUGCC | | [4419-4437 |
| 393 | GAGUACAAGUCAGGACAGU | ACUGUCCUGACUUGUACUC | | [4089-4107 |
| 394 | CCCAAGGCUUUGAGUACAA | UUGUACUCAAAGCCUUGGG | | [4078-4096 |
| 395 | CGCAGUCAAUGUCUACAUC | GAUGUAGACAUUGACUGCG | | [3638-3656 |
| 396 | ACGCAGUCAAUGUCUACAU | AUGUAGACAUUGACUGCGU | | [3637-3655 |
| 397 | AGGUGGAGGUGGAAAUGGU | ACCAUUUCCACCUCCACCU | | [3038-3056 |
| 398 | UGCCUUCCCUUAGUGAGUC | GACUCACUAAGGGAAGGCA | | [2022-2040 |
| 399 | CCAGUUUUGGUCCUACUAA | UUAGUAGGACCAAAACUGG | | [6114-6132] |
| 400 | AUCGGGCUCUUUCCUAAAA | UUUUAGGAAAGAGCCCGAU | | [5911-5929] |
| 401 | CUCUGCCAGAUAUUCAGUA | UACUGAAUAUCUGGCAGAG | | [5685-5703] |
| 402 | AAAAGGAGCAGCCCUACUU | AAGUAGGGCUGCUCCUUUU | | [5484-5502] |
| 403 | GGAGUAUACAGGAUACCGU | ACGGUAUCCUGUAUACUCC | | [1136-1154 |
| 404 | GCAGUCAAUGUCUACAUCU | AGAUGUAGACAUUGACUGC | | [3639-3657 |
| 405 | GCAGCUCUGUGUCAAAGGU | ACCUUUGACACAGAGCUGC | | [3017-3035 |
| 406 | CUUCUUCAGACACCUUUUU | AAAAAGGUGUCUGAAGAAG | | [2513-2531 |
| 407 | CUGCUGCUCAAGAUCCCUA | UAGGGAUCUUGAGCAGCAG | | [2319-2337 |
| 408 | GGUCGCUGUUAUCAACAUU | AAUGUUGAUAACAGCGACC | | [1835-1853 |
| 409 | UCCGGAAGGUCCUGAACAA | UUGUUCAGGACCUUCCGGA | | [1261-1279 |
| 410 | GGUGAAGGCAAGAAGUAGA | UCUACUUCUUGCCUUCACC | | [107-125] |
| 411 | CAAAUCCAGUUUUGGUCCU | AGGACCAAAACUGGAUUUG | | [6109-6127] |
| 412 | UGAUGUGAUUUCUACCAAA | UUUGGUAGAAAUCACAUCA | | [6094-6112] |
| 413 | GUACAUGUUUUACCACCAC | GUGGUGGUAAAACAUGUAC | | [5976-5994] |
| 414 | UCAGCUCUUUAACUUUUCU | AGAAAAGUUAAAGAGCUGA | | [5881-5899] |
| 415 | GGAUCUCCCAAAGAAGAUA | UAUCUUCUUUGGGAGAUCC | | [5147-5165] |
| 416 | CCCGGAGUAUACAGGAUAC | GUAUCCUGUAUACUCCGGG | | [1133-1151 |
| 417 | CAAUUGCAGUGCAUGAUGA | UCAUCAUGCACUGCAAUUG | | [5047-5065] |
| 418 | UACAAUUGCAGUGCAUGAU | AUCAUGCACUGCAAUUGUA | | [5045-5063] |
| 419 | UUCUACAAUUGCAGUGCAU | AUGCACUGCAAUUGUAGAA | | [5042-5060] |
| 420 | AGAAAACACUCCCGGAGUA | UACUCCGGGAGUGUUUUCU | | [1123-1141 |
| 421 | ACCAGAACAUCGCUGUGUA | UACACAGCGAUGUUCUGGU | | [1081-1099 |
| 422 | CCGCUAUGUGCCUUUUGAU | AUCAAAAGGCACAUAGCGG | | [3548-3566 |
| 423 | CCAAAAGCUGCAGCAGUAC | GUACUGCUGCAGCUUUUGG | | [3278-3296 |
| 424 | GAGCUCUUCUGACUGGAUC | GAUCCAGUCAGAAGAGCUC | | [241-259 |
| 425 | CUCAGGUGCUGGACAUCAA | UUGAUGUCCAGCACCUGAG | Rat, Ms | [2533-2551 |
| 426 | UGAGCGAGAAGGAGCUAUU | AAUAGCUCCUUCUCGCUCA | | [2449-2467 |
| 427 | CCAUCACCAUCAUUGCUCU | AGAGCAAUGAUGGUGAUGG | | [1999-2017 |
| 428 | ACGUGCUGGUCGCUGUUAU | AUAACAGCGACCAGCACGU | | [1828-1846 |
| 429 | CGCUACUGGUUUGAGAACA | UGUUCUCAAACCAGUAGCG | Rat, Ms | [1749-1767 |
| 430 | AGGACAACAUAGUGGUUGA | UCAACCACUAUGUUGUCCU | | [1408-1426 |
| 431 | GUGAAGGCAAGAAGUAGAG | CUCUACUUCUUGCCUUCAC | | [108-126] |
| 432 | AGGUCCUGAACAAGGGUUU | AAACCCUUGUUCAGGACCU | | [1267-1285 |
| 433 | AAAUCCAGUUUUGGUCCUA | UAGGACCAAAACUGGAUUU | | [6110-6128] |
| 434 | CGGGCUCUUUCCUAAAAAG | CUUUUUAGGAAAGAGCCCG | | [5913-5931] |
| 435 | CAGUCAGGAUCUCCCAAAG | CUUUGGGAGAUCCUGACUG | | [5141-5159] |
| 436 | CCAAUAUUGGGAGAAUAUU | AAUAUUCUCCCAAUAUUGG | | [4981-4999] |
| 437 | CCCAAUAUUGGGAGAAUAU | AUAUUCUCCCAAUAUUGGG | | [4980-4998] |
| 438 | GCUUCCAGUAUCCUGCCUU | AAGGCAGGAUACUGGAAGC | | [4871-4889] |
| 439 | CCACCAUGCUAUACAUCUG | CAGAUGUAUAGCAUGGUGG | | [4588-4606 |
| 440 | GCAGCCAAAUGCUGUGUAA | UUACACAGCAUUUGGCUGC | | [4420-4438 |
| 441 | UCAACCGCUAUGUGCCUUU | AAAGGCACAUAGCGGUUGA | | [3544-3562 |
| 442 | GCGUCUCCUUCAUGUUCUC | GAGAACAUGAAGGAGACGC | | [3469-3487 |
| 443 | CUGGACUGAAGAAGAGGUU | AACCUCUUCUUCAGUCCAG | | [3181-3199 |
| 444 | AGGUGGUGGAGUCUAUGUU | AACAUAGACUCCACCACCU | | [2908-2926 |
| 445 | GGAUGAGAAUGGCUUCCUC | GAGGAAGCCAUUCUCAUCC | | [2810-2828 |
| 446 | UACCAUGUAUGACCUGGAU | AUCCAGGUCAUACAUGGUA | Rat, Ms | [2795-2813 |
| 447 | UGCAGCAGGUCAACCUCAU | AUGAGGUUGACCUGCUGCA | | [2272-2290 |
| 448 | AGCUACAAAAGAAACUCAA | UUGAGUUUCUUUUGUAGCU | | [2089-2107 |
| 449 | GAGUCUGCUUCUCUCUGGA | UCCAGAGAGAAGCAGACUC | | [2036-2054 |
| 450 | CCUCAACCUAAGCAGCUCA | UGAGCUGCUUAGGUUGAGG | | [1908-1926 |
| 451 | GGUUUGAGAACACCAGGAA | UUCCUGGUGUUCUCAAACC | | [1756-1774 |
| 452 | CCUGGCAAAUUCUCCCGUA | UACGGGAGAAUUUGCCAGG | | [1452-1470 |
| 453 | GGGUUUUCAAAGCUCCCAA | UUGGGAGCUUUGAAAACCC | | [1280-1298 |
| 454 | GAUGUGAUUUCUACCAAAU | AUUUGGUAGAAAUCACAUC | | [6095-6113] |
| 455 | CCCAGGUCAAGACAUAGAA | UUCUAUGUCUUGACCUGGG | | [5994-6012] |
| 456 | CAGCUCUUUAACUUUUCUU | AAGAAAAGUUAAAGAGCUG | | [5882-5900] |
| 457 | CUCAGCUCUUUAACUUUUC | GAAAAGUUAAAGAGCUGAG | | [5880-5898] |
| 458 | ACUCAGCUCUUUAACUUUU | AAAAGUUAAAGAGCUGAGU | | [5879-5897] |
| 459 | GAAGCUGUGAUGCUUAGAA | UUCUAAGCAUCACAGCUUC | | [5411-5429] |
| 460 | CCCAGCUUCCUGCAGAAAA | UUUUCUGCAGGAAGCUGGG | Rat, Ms | [1110-1128 |
| 461 | CUCAGGACCACCAUGCUAU | AUAGCAUGGUGGUCCUGAG | | [4581-4599 |
| 462 | UGGUGUCUGUGCACAUUUA | UAAAUGUGCACAGACACCA | | [4534-4552 |
| 463 | CCAGAACAUCGCUGUGUAU | AUACACAGCGAUGUUCUGG | | [1082-1100 |
| 464 | UCAGGAGUGGCAUAAAUUU | AAAUUUAUGCCACUCCUGA | | [4316-4334 |
| 465 | CCACAUCUACUUCCUGGUC | GACCAGGAAGUAGAUGUGG | Rat, Ms | [3941-3959 |
| 466 | GCAGCUAUGCUCUGAUCCA | UGGAUCAGAGCAUAGCUGC | | [3910-3928 |
| 467 | ACCGCUAUGUGCCUUUUGA | UCAAAAGGCACAUAGCGGU | | [3547-3565 |
| 468 | GAGGCUUCCUAGCCCAAAA | UUUUGGGCUAGGAAGCCUC | | [3265-3283 |
| 469 | ACAAGGAGGAGCUGACAUG | CAUGUCAGCUCCUCCUUGU | | [2947-2965 |
| 470 | CCAACAACUGCCUGUCCAA | UUGGACAGGCAGUUGUUGG | Rat, Ms | [2875-2893 |
| 471 | AGAAUGGCUUCCUCUCCAA | UUGGAGAGGAAGCCAUUCU | | [2815-2833 |
| 472 | CCUGGUGGUCUUCAUGAAA | UUUCAUGAAGACCACCAGG | Ms | [2744-2762 |
| 473 | UGCUCAAGAUCCCUAAGGA | UCCUUAGGGAUCUUGAGCA | | [2323-2341 |
| 474 | CUGUCCAACAACCGAGGAU | AUCCUCGGUUGUUGGACAG | | [2292-2310 |
| 475 | CAACCUAAGCAGCUCACAA | UUGUGAGCUGCUUAGGUUG | | [1911-1929 |
| 476 | CUCAACCUAAGCAGCUCAC | GUGAGCUGCUUAGGUUGAG | | [1909-1927 |
| 477 | AGAGCACAGAAGCAAAGAU | AUCUUUGCUUCUGUGCUCU | | [123-141] |
| 478 | ACAACAUAGUGGUUGAAGA | UCUUCAACCACUAUGUUGU | | [1411-1429 |
| 479 | GAGGACAACAUAGUGGUUG | CAACCACUAUGUUGUCCUC | | [1407-1425 |
| 480 | GUCCUGAACAAGGGUUUUC | GAAAACCCUUGUUCAGGAC | | [1269-1287 |
| 481 | GGUCCUGAACAAGGGUUUU | AAAACCCUUGUUCAGGACC | | [1268-1286 |
| 482 | GUCUACAUGAGAAAUGCCA | UGGCAUUUCUCAUGUAGAC | | [1233-1251 |
| 483 | GCACCAGGAAACAAAGAUG | CAUCUUUGUUUCCUGGUGC | | [5545-5563] |
| 484 | CAGAAGCUGUGAUGCUUAG | CUAAGCAUCACAGCUUCUG | | [5409-5427] |
| 485 | GCAGAAGCUGUGAUGCUUA | UAAGCAUCACAGCUUCUGC | | [5408-5426] |
| 486 | CCGGAGUAUACAGGAUACC | GGUAUCCUGUAUACUCCGG | | [1134-1152 |
| 487 | UCCCGGAGUAUACAGGAUA | UAUCCUGUAUACUCCGGGA | | [1132-1150 |
| 488 | GUUCUACAAUUGCAGUGCA | UGCACUGCAAUUGUAGAAC | | [5041-5059] |
| 489 | UCCCAAUAUUGGGAGAAUA | UAUUCUCCCAAUAUUGGGA | | [4979-4997] |
| 490 | CUCCAGGAAUGACCAAGAA | UUCUUGGUCAUUCCUGGAG | | [4759-4777 |
| 491 | UGCACAUUUAUGUCACCCA | UGGGUGACAUAAAUGUGCA | | [4543-4561 |
| 492 | AGGAGUGGCAUAAAUUUGA | UCAAAUUUAUGCCACUCCU | | [4318-4336 |
| 493 | CCACGCAGUCAAUGUCUAC | GUAGACAUUGACUGCGUGG | | [3635-3653 |
| 494 | GCCAUUUUGCACAGUGCUG | CAGCACUGUGCAAAAUGGC | | [3615-3633 |
| 495 | UGCGAGAGACUUUCCUCAA | UUGAGGAAAGUCUCUCGCA | | [3529-3547 |
| 496 | CCCAAAAGCUGCAGCAGUA | UACUGCUGCAGCUUUUGGG | | [3277-3295 |
| 497 | GCUGGUUUAACAACCUGAG | CUCAGGUUGUUAAACCAGC | | [322-340 |
| 498 | UGCGAUCCUUCAUCGAGAU | AUCUCGAUGAAGGAUCGCA | | [2854-2872 |
| 499 | AGGACGAAUUCUUCACCAU | AUGGUGAAGAAUUCGUCCU | | [2833-2851 |
| 500 | AGAGGAUAAGUCCCGUCUA | UAGACGGGACUUAUCCUCU | | [2771-2789 |

TABLE A10
DUOX2 - 19-mer oligomer pairs

| | | | | |
|---|---|---|---|---|
| | | | Other | Human-132566531 |
| No. | Sense siRNA | AntiSense siRNA | Sp | ORF: 204-4850 |

| | | | | |
|---|---|---|---|---|
| 1 | GAGAUAUCUUUAAACAAAA | UUUUGUUUAAAGAUAUCUC | | [3061-3079] |
| 2 | GGAGUGAUCUCAACCCUAA | UUAGGGUUGAGAUCACUCC | | [1570-1588] |
| 3 | CGCUAUGACGGCUGGUUUA | UAAACCAGCCGUCAUAGCG | | [312-330] |
| 4 | GAGGAUAAGUCCCGUCUAA | UUAGACGGGACUUAUCCUC | | [2772-2790] |
| 5 | CAGGAUCUCCCAAAGAAGA | UCUUCUUUGGGAGAUCCUG | | [5145-5163] |
| 6 | GAGGUAUAAUAAAUAAUCA | UGAUUAUUUAUUAUACCUC | | [5956-5974] |
| 7 | ACAAGAAGCUACAAAAGAA | UUCUUUUGUAGCUUCUUGU | | [2083-2101] |
| 8 | CAAUUAUACAAAUGAGAAA | UUUCUCAUUUGUAUAAUUG | | [5008-5026] |
| 9 | AACCCAACACCACCCUAAA | UUUAGGGUGGUGUUGGGUU | | [165-183] |
| 10 | CCUGAACAAGGGUUUUCAA | UUGAAAACCCUUGUUCAGG | | [1271-1289] |
| 11 | GGACCACCAUGCUAUACAU | AUGUAUAGCAUGGUGGUCC | | [4585-4603] |
| 12 | CAUCGGGCUCUUUCCUAAA | UUUAGGAAAGAGCCCGAUG | | [5910-5928] |
| 13 | CCAAGGACCCAGAGAGAAA | UUUCUCUCUGGGUCCUUGG | | [37-55] |
| 14 | GAGUGAUCUCAACCCUAAU | AUUAGGGUUGAGAUCACUC | | [1571-1589] |
| 15 | UGACAAGUGUGGAGGUGAA | UUCACCUCCACACUUGUCA | | [5240-5258] |
| 16 | GUAAGAAGAUCUACUUCAU | AUGAAGUAGAUCUUCUUAC | Rat, Ms | [4435-4453] |
| 17 | GCAAAGAUUUUAAGAGGAA | UUCCUCUUAAAAUCUUUGC | | [134-152] |
| 18 | GGCAAUCUCUCCAUCAGAA | UUCUGAUGGAGAGAUUGCC | | [4923-4941] |
| 19 | CCUUCAUCGAGAUCUCCAA | UUGGAGAUCUCGAUGAAGG | Rat | [2860-2878] |
| 20 | CAAAGAUUUUAAGAGGAAA | UUUCCUCUUAAAAUCUUUG | | [135-153] |
| 21 | CCACCCAGGUCAAGACAUA | UAUGUCUUGACCUGGGUGG | | [5991-6009] |
| 22 | CAUCGAGGUAUAAUAAAUA | UAUUUAUUAUACCUCGAUG | | [5952-5970] |
| 23 | AGAGAUAUCUUUAAACAAA | UUUGUUUAAAGAUAUCUCU | | [3060-3078] |
| 24 | AGAAGGAGCUAUUUAGGAA | UUCCUAAAUAGCUCCUUCU | | [2455-2473] |
| 25 | CCAUGAUGCGAUCCUUCAU | AUGAAGGAUCGCAUCAUGG | | [2848-2866] |
| 26 | GGAGAUUGAAGACAUCCGA | UCGGAUGUCUUCAAUCUCC | | [1793-1811] |
| 27 | ACAGAAGCAAAGAUUUUAA | UUAAAAUCUUUGCUUCUGU | | [128-146] |
| 28 | CUCCAAGAAGGAGAUUGAA | UUCAAUCUCCUUCUUGGAG | | [1784-1802] |
| 29 | GAGAUUGAAGACAUCCGAA | UUCGGAUGUCUUCAAUCUC | | [1794-1812] |
| 30 | GGAGCAGUCCCAUCAUCAU | AUGAUGAUGGGACUGCUCC | | [2176-2194] |
| 31 | GGCAGGAGACUAUGUUCUA | UAGAACAUAGUCUCCUGCC | | [5028-5046] |
| 32 | GGCUGUUCUCCAAGAAGGA | UCCUUCUUGGAGAACAGCC | | [1777-1795] |
| 33 | GGAAUGGGCUGUUCUCCAA | UUGGAGAACAGCCCAUUCC | Ms | [1771-1789] |
| 34 | GUCAGGAUCUCCCAAAGAA | UUCUUUGGGAGAUCCUGAC | | [5143-5161] |
| 35 | AAGAGAGCGUGAAGAAGGA | UCCUUCUUCACGCUCUCUU | | [2107-2125] |
| 36 | CAACAUAGUGGUUGAAGAU | AUCUUCAACCACUAUGUUG | | [1412-1430] |
| 37 | CAGGAAUGACCAAGAAUGU | ACAUUCUUGGUCAUUCCUG | | [4762-4780] |
| 38 | AGCGCUUCGUGGAGAACUA | UAGUUCUCCACGAAGCGCU | | [3298-3316] |
| 39 | CUCCCAAAGAAGAUAAAGA | UCUUUAUCUUCUUUGGGAG | | [5151-5169] |
| 40 | GAACCCAACACCACCCUAA | UUAGGGUGGUGUUGGGUUC | | [164-182] |
| 41 | GGCUGAGAAUCUCCUCAGA | UCUGAGGAGAUUCUCAGCC | | [5382-5400] |
| 42 | CCAAGAAGGAGAUUGAAGA | UCUUCAAUCUCCUUCUUGG | | [1786-1804] |
| 43 | GACCGCUACUGGUUUGAGA | UCUCAAACCAGUAGCGGUC | | [1746-1764] |
| 44 | AGUCGGGAUUCCAGGACAA | UUGUCCUGGAAUCCCGACU | | [2932-2950] |
| 45 | UCAGGACCACCAUGCUAUA | UAUAGCAUGGUGGUCCUGA | | [4582-4600] |
| 46 | GGAAGGCUGUGACAAAGCA | UGCUUUGUCACAGCCUUCC | | [2470-2488] |
| 47 | GGAAGAAGGUGGAGAUCAG | CUGAUCUCCACCUUCUUCC | Rat, Ms | [4003-4021] |
| 48 | UGCUGGUCGCUGUUAUCAA | UUGAUAACAGCGACCAGCA | | [1831-1849] |
| 49 | CUGGAUACCCAAAGCUGUA | UACAGCUUUGGGUAUCCAG | | [4270-4288] |
| 50 | GGAGGUGGAAAUGGUAUUA | UAAUACCAUUUCCACCUCC | | [3042-3060] |
| 51 | AUCGAGGUAUAAUAAAUAA | UUAUUUAUUAUACCUCGAU | | [5953-5971] |
| 52 | CCUUCGAGCCCUUCUUCAA | UUGAAGAAGGGCUCGAAGG | | [4684-4702] |
| 53 | AGUAGAGCACAGAAGCAAA | UUUGCUUCUGUGCUCUACU | | [120-138] |
| 54 | AUUCAGUACUCGAUCUCAA | UUGAGAUCGAGUACUGAAU | | [5696-5714] |
| 55 | CUGAGAUGAGCGAGAAGGA | UCCUUCUCGCUCAUCUCAG | | [2443-2461] |
| 56 | GACCCAGAGAGAAAGGUGA | UCACCUUUCUCUCUGGGUC | | [42-60] |
| 57 | UUAGGAAGGCUGUGACAAA | UUUGUCACAGCCUUCCUAA | | [2467-2485] |
| 58 | CCCAGAUUUCGGAGUUGGA | UCCAACUCCGAAAUCUGGG | | [1390-1408] |
| 59 | GACAAUUAUACAAAUGAGA | UCUCAUUUGUAUAAUUGUC | | [5006-5024] |
| 60 | GAUUGAAGACAUCCGAAAU | AUUUCGGAUGUCUUCAAUC | | [1796-1814] |
| 61 | UCAUCGGGCUCUUUCCUAA | UUAGGAAAGAGCCCGAUGA | | [5909-5927] |
| 62 | GAGAAGGAGCUAUUUAGGA | UCCUAAAUAGCUCCUUCUC | | [2454-2472] |
| 63 | GAGGAAAGAAGACAUCUGA | UCAGAUGUCUUCUUUCCUC | | [147-165] |
| 64 | GUAUUAGAGAUAUCUUUAA | UUAAAGAUAUCUCUAAUAC | | [3055-3073] |
| 65 | GGAAAUGGUAUUAGAGAUA | UAUCUCUAAUACCAUUUCC | | [3048-3066] |
| 66 | GUGGAAAUGGUAUUAGAGA | UCUCUAAUACCAUUUCCAC | | [3046-3064] |
| 67 | CAGACAAUUAUACAAAUGA | UCAUUUGUAUAAUUGUCUG | | [5004-5022] |
| 68 | CCAGCUUCCUGCAGAAAAC | GUUUUCUGCAGGAAGCUGG | Rat, Ms | [1111-1129] |
| 69 | UGACUUCCACAAAAUCGAU | AUCGAUUUUGUGGAAGUCA | | [5304-5322] |
| 70 | GACGAAUUCUUCACCAUGA | UCAUGGUGAAGAAUUCGUC | | [2835-2853] |
| 71 | GCAUAAAUUUGAGGUGUCA | UGACACCUCAAAUUUAUGC | | [4325-4343] |
| 72 | AGAUAAAGACCACUCCUCA | UGAGGAGUGGUCUUUAUCU | | [5161-5179] |
| 73 | GAAGACAUCCGAAAUACCA | UGGUAUUUCGGAUGUCUUC | | [1800-1818] |
| 74 | UCAAAGACCUGGUCUUCAA | UUGAAGACCAGGUCUUUGA | Rat, Ms | [4390-4408] |
| 75 | AGAUUUUAAGAGGAAAGAA | UUCUUUCCUCUUAAAAUCU | | [138-156] |
| 76 | AGAUUGAAGACAUCCGAAA | UUUCGGAUGUCUUCAAUCU | | [1795-1813] |
| 77 | GGUUGAAGAUCUGAGGGAU | AUCCCUCAGAUCUUCAACC | | [1421-1439] |
| 78 | CAAUCAGGACGCACUCUCA | UGAGAGUGCGUCCUGAUUG | | [275-293] |
| 79 | UGUGCUUGACUUCUUUGAA | UUCAAAGAAGUCAAGCACA | | [1961-1979] |
| 80 | UGACCCAGACAAUUAUACA | UGUAUAAUUGUCUGGGUCA | | [4999-5017] |
| 81 | UAGAGAUAUCUUUAAACAA | UUGUUUAAAGAUAUCUCUA | | [3059-3077] |
| 82 | GAGACUAUGUUCUACAAUU | AAUUGUAGAACAUAGUCUC | | [5033-5051] |
| 83 | UCCUGGUGGUCUUCAUGAA | UUCAUGAAGACCACCAGGA | Ms | [2743-2761] |
| 84 | GGUGGAAAUGGUAUUAGAG | CUCUAAUACCAUUUCCACC | | [3045-3063] |
| 85 | GAGGGUGAAGGCAAGAAGU | ACUUCUUGCCUUCACCCUC | | [104-122] |
| 86 | CGCUGCAAGAGAAGAUGCA | UGCAUCUUCUCUUGCAGCG | | [3244-3262] |
| 87 | GAUCUCCCAAAGAAGAUAA | UUAUCUUCUUUGGGAGAUC | | [5148-5166] |
| 88 | GGUAUUAGAGAUAUCUUUA | UAAAGAUAUCUCUAAUACC | | [3054-3072] |
| 89 | CGAGAAGGAGCUAUUUAGG | CCUAAAUAGCUCCUUCUCG | | [2453-2471] |
| 90 | GAGCAGUUCUUCUCUACCA | UGGUAGAGAAGAACUGCUC | | [1200-1218] |
| 91 | ACAAUUAUACAAAUGAGAA | UUCUCAUUUGUAUAAUUGU | | [5007-5025] |
| 92 | CUACAAUUGCAGUGCAUGA | UCAUGCACUGCAAUUGUAG | | [5044-5062] |
| 93 | AGGAAUGACCAAGAAUGUA | UACAUUCUUGGUCAUUCCU | | [4763-4781] |
| 94 | GGUGUCUGUGCACAUUUAU | AUAAAUGUGCACAGACACC | | [4535-4553] |
| 95 | ACCGCUACUGGUUUGAGAA | UUCUCAAACCAGUAGCGGU | | [1747-1765] |
| 96 | CCAGUAUCCUGCCUUCUCU | AGAGAAGGCAGGAUACUGG | | [4875-4893] |
| 97 | AGACAAUUAUACAAAUGAG | CUCAUUUGUAUAAUUGUCU | | [5005-5023] |
| 98 | CUGAACAAGGGUUUUCAAA | UUUGAAAACCCUUGUUCAG | | [1272-1290] |
| 99 | UAAUGUUUACCAUGUAUGA | UCAUACAUGGUAAACAUUA | Rat | [2788-2806] |
| 100 | UGUCAAAGGUGGAGGUGGA | UCCACCUCCACCUUUGACA | | [3026-3044] |
| 101 | UCUCCAAGAAGGAGAUUGA | UCAAUCUCCUUCUUGGAGA | | [1783-1801] |
| 102 | UAAUUGACUUCCACAAAAU | AUUUUGUGGAAGUCAAUUA | | [5300-5318] |
| 103 | GGCUGGUUUAACAACCUGA | UCAGGUUGUUAAACCAGCC | | [321-339] |
| 104 | AUCUCCCAAAGAAGAUAAA | UUUAUCUUCUUUGGGAGAU | | [5149-5167] |
| 105 | CAGCUUCCUGCAGAAAACA | UGUUUUCUGCAGGAAGCUG | Rat | [1112-1130] |
| 106 | AGGAGUGACCUACCUGCAA | UUGCAGGUAGGUCACUCCU | | [4049-4067] |
| 107 | CCCAGGACAUGUUUGUGGA | UCCACAAACAUGUCCUGGG | Ms | [2659-2677] |
| 108 | GAGCGUGAAGAAGGAAGCA | UGCUUCCUUCUUCACGCUC | | [2111-2129] |
| 109 | GAAGUAGAGCACAGAAGCA | UGCUUCUGUGCUCUACUUC | | [118-136] |
| 110 | AGGGCACCAGGAAACAAAG | CUUUGUUUCCUGGUGCCCU | | [5542-5560] |
| 111 | CCCAGAGAGAAAGGUGAGA | UCUCACCUUUCUCUCUGGG | | [44-62] |
| 112 | CACAAGAAGCUACAAAAGA | UCUUUUGUAGCUUCUUGUG | | [2082-2100] |
| 113 | GACCAAGAAUGUAGAGAAG | CUUCUCUACAUUCUUGGUC | | [4769-4787] |
| 114 | ACAUCGAGGUAUAAUAAAU | AUUUAUUAUACCUCGAUGU | | [5951-5969] |
| 115 | GCUGUGCUGGAUACCCAAA | UUUGGGUAUCCAGCACAGC | | [4264-4282] |
| 116 | GCUGGAUACCCAAAGCUGU | ACAGCUUUGGGUAUCCAGC | | [4269-4287] |
| 117 | UUACCACCACCCAGGUCAA | UUGACCUGGGUGGUGGUAA | | [5985-6003] |
| 118 | GAGCGAGAAGGAGCUAUUU | AAAUAGCUCCUUCUCGCUC | | [2450-2468] |
| 119 | ACAGUACCCAGGAGGUGAA | UUCACCUCCUGGGUACUGU | | [1348-1366] |
| 120 | ACCCAAAGCUGUACCUUGA | UCAAGGUACAGCUUUGGGU | | [4276-4294] |
| 121 | UGGAGUGAUCUCAACCCUA | UAGGGUUGAGAUCACUCCA | | [1569-1587] |
| 122 | ACAUCUGAACCCAACACCA | UGGUGUUGGGUUCAGAUGU | | [158-176] |
| 123 | AGCGAGAAGGAGCUAUUUA | UAAAUAGCUCCUUCUCGCU | | [2451-2469] |
| 124 | UGGAGAUCUUCUUCAGACA | UGUCUGAAGAAGAUCUCCA | Rat, Ms | [2506-2524] |
| 125 | CAUGAUGAUUAUAAGUCCA | UGGACUUAUAAUCAUCAUG | | [5058-5076] |
| 126 | CCUCCAGGAAUGACCAAGA | UCUUGGUCAUUCCUGGAGG | | [4758-4776] |
| 127 | UUGACUUCCACAAAAUCGA | UCGAUUUUGUGGAAGUCAA | | [5303-5321] |
| 128 | UCCAAGAGGUGGAGGAGAA | UUCUCCUCCACCUCUUGGA | | [4501-4519] |
| 129 | CCUGGAGAUCUUCUUCAGA | UCUGAAGAAGAUCUCCAGG | | [2504-2522] |
| 130 | GGUUUAACAACCUGAGGCA | UGCCUCAGGUUGUUAAACC | | [325-343] |
| 131 | UAUUGACCCAGACAAUUAU | AUAAUUGUCUGGGUCAAUA | | [4996-5014] |
| 132 | UGUAUGACCUGGAUGAGAA | UUCUCAUCCAGGUCAUACA | | [2800-2818] |
| 133 | UAAUCAUGUACAUGUUUUA | UAAAACAUGUACAUGAUUA | | [5969-5987] |
| 134 | AAGCUCUCAGGGUCUGCAA | UUGCAGACCCUGAGAGCUU | | [1297-1315] |
| 135 | GAACUGGAGUGAUCUCAAC | GUUGAGAUCACUCCAGUUC | | [1565-1583] |
| 136 | CCAUGUAUGACCUGGAUGA | UCAUCCAGGUCAUACAUGG | | [2797-2815] |
| 137 | UGGUCCCGGCAAUCAUCUA | UAGAUGAUUGCCGGGACCA | | [3955-3973] |
| 138 | AUGCGAUCCUUCAUCGAGA | UCUCGAUGAAGGAUCGCAU | | [2853-2871] |
| 139 | UUACAUCGAGGUAUAAUAA | UUAUUAUACCUCGAUGUAA | | [5949-5967] |
| 140 | UCAUGUACAUGUUUUACCA | UGGUAAAACAUGUACAUGA | | [5972-5990] |
| 141 | AAAAGCUGCAGCAGUACAA | UUGUACUGCUGCAGCUUUU | | [3280-3298] |
| 142 | UCAGGAUCUCCCAAAGAAG | CUUCUUUGGGAGAUCCUGA | | [5144-5162] |
| 143 | CGAGGUAUAAUAAAUAAUC | GAUUAUUUAUUAUACCUCG | | [5955-5973] |
| 144 | GCUGUAAAAUAUUUUACAU | AUGUAAAAUAUUUUACAGC | | [5936-5954] |
| 145 | UGUCCUCGACCAGUUUGUA | UACAAACUGGUCGAGGACA | | [1712-1730] |
| 146 | CUAAGCAGCUCACAACUGA | UCAGUUGUGAGCUGCUUAG | | [1915-1933] |
| 147 | AAGACAUCUGAACCCAACA | UGUUGGGUUCAGAUGUCUU | | [155-173] |
| 148 | GGAGUGACCUACCUGCAAU | AUUGCAGGUAGGUCACUCC | | [4050-4068] |
| 149 | UCUGGUUCCGCUACCACAA | UUGUGGUAGCGGAACCAGA | | [973-991] |
| 150 | GAGAGUUCCUGGACAUCCU | AGGAUGUCCAGGAACUCUC | | [2728-2746] |
| 151 | AGGAGCAGCCCUACUUCUA | UAGAAGUAGGGCUGCUCCU | | [5487-5505] |
| 152 | AGAAGGAGAUUGAAGACAU | AUGUCUUCAAUCUCCUUCU | | [1789-1807] |
| 153 | AGGAUACCGUCCUUUCCUA | UAGGAAAGGACGGUAUCCU | | [1145-1163] |
| 154 | CAUAGUGGUUGAAGAUCUG | CAGAUCUUCAACCACUAUG | | [1415-1433] |
| 155 | CAUCCUGUCCAACAACCGA | UCGGUUGUUGGACAGGAUG | | [2288-2306] |
| 156 | CCCAAAGCUGUACCUUGAU | AUCAAGGUACAGCUUUGGG | | [4277-4295] |
| 157 | CGCUGUUAUCAACAUUGAC | GUCAAUGUUGAUAACAGCG | | [1838-1856] |
| 158 | AGUUUUGGUCCUACUAAAU | AUUUAGUAGGACCAAAACU | | [6116-6134] |
| 159 | CAUUGUCCUCGACCAGUUU | AAACUGGUCGAGGACAAUG | | [1709-1727] |
| 160 | GCACAGAAGCAAAGAUUUU | AAAAUCUUUGCUUCUGUGC | | [126-144] |
| 161 | AGGCUUUGAGUACAAGUCA | UGACUUGUACUCAAAGCCU | | [4082-4100] |
| 162 | CAGAAAGUGCUGAACCGGA | UCCGGUUCAGCACUUUCUG | | [4620-4638] |
| 163 | CUGAUGUGAUUUCUACCAA | UUGGUAGAAAUCACAUCAG | | [6093-6111] |
| 164 | AGAAGCAAAGAUUUUAAGA | UCUUAAAAUCUUUGCUUCU | | [130-148] |
| 165 | CCACAUCUCUAAUUGACUU | AAGUCAAUUAGAGAUGUGG | | [5291-5309] |
| 166 | UAUUUUACAUCGAGGUAUA | UAUACCUCGAUGUAAAAUA | | [5945-5963] |
| 167 | GAGAAAAGGCAGGAGACUA | UAGUCUCCUGCCUUUUCUC | | [5021-5039] |
| 168 | AAAGCUGAGCUGUAAAAUA | UAUUUUACAGCUCAGCUUU | | [5928-5946] |
| 169 | AAAACACUCCCGGAGUAUA | UAUACUCCGGGAGUGUUUU | | [1125-1143] |
| 170 | GAAGGAGAUUGAAGACAUC | GAUGUCUUCAAUCUCCUUC | | [1790-1808] |
| 171 | UCAAGAUCCCUAAGGAGUA | UACUCCUUAGGGAUCUUGA | | [2326-2344] |
| 172 | CAGUAUCCUGCCUUCUCUU | AAGAGAAGGCAGGAUACUG | | [4876-4894] |
| 173 | UGUCGAGUCUCGUUCAUCA | UGAUGAACGAGACUCGACA | Ms | [3087-3105] |
| 174 | GGCUCUUUCCUAAAAAGCU | AGCUUUUUAGGAAAGAGCC | | [5915-5933] |
| 175 | GAGAAUAUUGACCCAGACA | UGUCUGGGUCAAUAUUCUC | | [4991-5009] |
| 176 | CAAUGUCUACAUCUUCUCA | UGAGAAGAUGUAGACAUUG | | [3644-3662] |
| 177 | AGGAUCUCCCAAAGAAGAU | AUCUUCUUUGGGAGAUCCU | | [5146-5164] |
| 178 | CAAUAGUGCUUGUCUCCGA | UCGGAGACAAGCACUAUUG | | [5265-5283] |
| 179 | UUUUGGUCCUACUAAAUAU | AUAUUUAGUAGGACCAAAA | | [6118-6136] |
| 180 | AUGACGGCUGGUUUAACAA | UUGUUAAACCAGCCGUCAU | | [316-334] |
| 181 | UUCAGUACUCGAUCUCAAU | AUUGAGAUCGAGUACUGAA | | [5697-5715] |
| 182 | UCAAAGAGAGCGUGAAGAA | UUCUUCACGCUCUCUUUGA | | [2104-2122] |
| 183 | GUUUUGGUCCUACUAAAUA | UAUUUAGUAGGACCAAAAC | | [6117-6135] |
| 184 | GUACCUUGAUGGACCGUUU | AAACGGUCCAUCAAGGUAC | | [4286-4304] |
| 185 | GUGGAGGUGGAAAUGGUAU | AUACCAUUUCCACCUCCAC | | [3040-3058] |
| 186 | CGUGUUUGCAGAUCGUGCU | AGCACGAUCUGCAAACACG | | [3365-3383] |
| 187 | GACAUUUGAGGGAGGAGGA | UCCUCCUCCCUCAAAUGUC | | [5349-5367] |
| 188 | CAAGAUCCCUAAGGAGUAU | AUACUCCUUAGGGAUCUUG | | [2327-2345] |
| 189 | AACGCAUCCUGGAGAUCUU | AAGAUCUCCAGGAUGCGUU | | [2497-2515] |
| 190 | AGGUGAAAAGGUACAAUAG | CUAUUGUACCUUUUCACCU | | [5252-5270] |
| 191 | CAUGUUUUACCACCACCCA | UGGGUGGUGGUAAAACAUG | | [5979-5997] |
| 192 | AGCAGUUCUUCUCUACCAU | AUGGUAGAGAAGAACUGCU | | [1201-1219] |
| 193 | UCGCUGUUAUCAACAUUGA | UCAAUGUUGAUAACAGCGA | | [1837-1855] |
| 194 | CAGGGUCUGCAACAACUAC | GUAGUUGUUGCAGACCCUG | Rat | [1304-1322] |
| 195 | UCAUCGAGAUCUCCAACAA | UUGUUGGAGAUCUCGAUGA | Rat | [2863-2881] |
| 196 | GGGUGAAGGCAAGAAGUAG | CUACUUCUUGCCUUCACCC | | [106-124] |
| 197 | UUUGGUCCUACUAAAUAUA | UAUAUUUAGUAGGACCAAA | | [6119-6137] |
| 198 | UGAGCUGUAAAAUAUUUUA | UAAAAUAUUUUACAGCUCA | | [5933-5951] |
| 199 | GUUUGAGAACACCAGGAAU | AUUCCUGGUGUUCUCAAAC | | [1757-1775] |
| 200 | UCGAGGUAUAAUAAAUAAU | AUUAUUUAUUAUACCUCGA | | [5954-5972] |
| 201 | AGGACCUAUCCCAGCUAGA | UCUAGCUGGGAUAGGUCCU | | [1633-1651] |
| 202 | GAUCGUGCUUACUACUAUG | CAUAGUAGUAAGCACGAUC | | [3375-3393] |
| 203 | UUUCGGAGUUGGAGGACAA | UUGUCCUCCAACUCCGAAA | | [1396-1414] |
| 204 | GAUUAUAAGUCCACCUGUU | AACAGGUGGACUUAUAAUC | | [5064-5082] |
| 205 | AAAUAAUCAUGUACAUGUU | AACAUGUACAUGAUUAUUU | | [5966-5984] |
| 206 | CAAGGCUUUGAGUACAAGU | ACUUGUACUCAAAGCCUUG | | [4080-4098] |
| 207 | AACUCUGUCUCCAGGUCUA | UAGACCUGGAGACAGAGUU | | [5451-5469] |
| 208 | UGCUCUAUGCCCUGCUCAU | AUGAGCAGGGCAUAGAGCA | | [3883-3901] |
| 209 | AAAGCUGUACCUUGAUGGA | UCCAUCAAGGUACAGCUUU | | [4280-4298] |
| 210 | UGCAGAAAACACUCCCGGA | UCCGGGAGUGUUUUCUGCA | | [1120-1138] |
| 211 | AGAGGAGCAGUCCCAUCAU | AUGAUGGGACUGCUCCUCU | | [2173-2191] |
| 212 | CAUCUAUGGAGGUGACAAG | CUUGUCACCUCCAUAGAUG | | [3968-3986] |
| 213 | AGAACUACCGGAGGCACAU | AUGUGCCUCCGGUAGUUCU | | [3310-3328] |
| 214 | UAUGACGGCUGGUUUAACA | UGUUAAACCAGCCGUCAUA | | [315-333] |
| 215 | ACAUCAUCCAAGAGGUGGA | UCCACCUCUUGGAUGAUGU | | [4495-4513] |
| 216 | GUAAAAUAUUUUACAUCGA | UCGAUGUAAAAUAUUUUAC | | [5939-5957] |
| 217 | AGAGAGCGUGAAGAAGGAA | UUCCUUCUUCACGCUCUCU | | [2108-2126] |
| 218 | GCUGGUCGCUGUUAUCAAC | GUUGAUAACAGCGACCAGC | | [1832-1850] |
| 219 | GAGCACAGAAGCAAAGAUU | AAUCUUUGCUUCUGUGCUC | | [124-142] |
| 220 | CGUCUAAUGUUUACCAUGU | ACAUGGUAAACAUUAGACG | | [2784-2802] |
| 221 | ACCAGUCAGGAUCUCCCAA | UUGGGAGAUCCUGACUGGU | | [5139-5157] |
| 222 | CUUUGAGUACAAGUCAGGA | UCCUGACUUGUACUCAAAG | | [4085-4103] |
| 223 | AGUCAGGAUCUCCCAAAGA | UCUUUGGGAGAUCCUGACU | | [5142-5160] |
| 224 | UACAUCGAGGUAUAAUAAA | UUUAUUAUACCUCGAUGUA | | [5950-5968] |
| 225 | UGAUGCGAUCCUUCAUCGA | UCGAUGAAGGAUCGCAUCA | | [2851-2869] |
| 226 | GUCUGCAACAACUACUGGA | UCCAGUAGUUGUUGCAGAC | Rat | [1308-1326] |
| 227 | GAAGAUAAAGACCACUCCU | AGGAGUGGUCUUUAUCUUC | | [5159-5177] |
| 228 | UUUACAUCGAGGUAUAAUA | UAUUAUACCUCGAUGUAAA | | [5948-5966] |
| 229 | CCAGCUGUCAUUUCCGGAA | UUCCGGAAAUGACAGCUGG | Ms | [1249-1267] |
| 230 | AUGGGCUGUUCUCCAAGAA | UUCUUGGAGAACAGCCCAU | | [1774-1792] |
| 231 | AAAGUGUCAGCAACUUUGA | UCAAAGUUGCUGACACUUU | | [5521-5539] |
| 232 | GCAGAUCGUGCUUACUACU | AGUAGUAAGCACGAUCUGC | | [3372-3390] |
| 233 | AACCAGUCAGGAUCUCCCA | UGGGAGAUCCUGACUGGUU | | [5138-5156] |
| 234 | AGCUGUAAAAUAUUUUACA | UGUAAAAUAUUUUACAGCU | | [5935-5953] |
| 235 | AUUGACCCAGACAAUUAUA | UAUAAUUGUCUGGGUCAAU | | [4997-5015] |
| 236 | UACAAAAGAAACUCAAAGA | UCUUUGAGUUUCUUUUGUA | | [2092-2110] |
| 237 | CUGCCUUCCCUUAGUGAGU | ACUCACUAAGGGAAGGCAG | | [2021-2039] |
| 238 | CUCUAUGCCCUGCUCAUCA | UGAUGAGCAGGGCAUAGAG | | [3885-3903] |
| 239 | UAAAAAGCUGAGCUGUAAA | UUUACAGCUCAGCUUUUUA | | [5925-5943] |
| 240 | CAUGUGCCGCAACCUCAUA | UAUGAGGUUGCGGCACAUG | | [3503-3521] |
| 241 | CCUGUACAACCAGGACCUA | UAGGUCCUGGUUGUACAGG | | [1622-1640] |
| 242 | UCACCAUGAUGCGAUCCUU | AAGGAUCGCAUCAUGGUGA | | [2845-2863] |
| 243 | GACCUGGUCUUCAAGUCAU | AUGACUUGAAGACCAGGUC | | [4395-4413] |
| 244 | UCUCUAAUUGACUUCCACA | UGUGGAAGUCAAUUAGAGA | | [5296-5314] |
| 245 | CUGCGAGAGACUUUCCUCA | UGAGGAAAGUCUCUCGCAG | | [3528-3546] |
| 246 | GUGCAUGAUGAUUAUAAGU | ACUUAUAAUCAUCAUGCAC | | [5055-5073] |
| 247 | CAAGGAACUGGAGUGAUCU | AGAUCACUCCAGUUCCUUG | | [1561-1579] |
| 248 | GGAUAAGUCCCGUCUAAUG | CAUUAGACGGGACUUAUCC | | [2774-2792] |
| 249 | CUUUUCUUUUCCUUUUUCA | UGAAAAAGGAAAAGAAAAG | | [5893-5911] |
| 250 | GAAUGAUGGGUCCAAGCUU | AAGCUUGGACCCAUCAUUC | Rat | [3710-3728] |
| 251 | CAAAAGCUGCAGCAGUACA | UGUACUGCUGCAGCUUUUG | | [3279-3297] |
| 252 | AGCUGGUCACCUUACGCAA | UUGCGUAAGGUGACCAGCU | | [5834-5852] |
| 253 | UGUGCCUUUUGAUGCCGCA | UGCGGCAUCAAAAGGCACA | | [3554-3572] |
| 254 | GCUCUUUAACUUUUCUUUU | AAAAGAAAAGUUAAAGAGC | | [5884-5902] |
| 255 | GUGCAGAAGCUGUGAUGCU | AGCAUCACAGCUUCUGCAC | | [5406-5424] |
| 256 | GCAAUCUCUCCAUCAGAAU | AUUCUGAUGGAGAGAUUGC | | [4924-4942] |
| 257 | UGGUAUUAGAGAUAUCUUU | AAAGAUAUCUCUAAUACCA | | [3053-3071] |
| 258 | GACUUCCUGCCCUUAGCAA | UUGCUAAGGGCAGGAAGUC | | [5113-5131] |
| 259 | CGGAAGAAGGUGGAGAUCA | UGAUCUCCACCUUCUUCCG | Rat, Ms | [4002-4020] |
| 260 | CAAUCUGAACAGUACCCAG | CUGGGUACUGUUCAGAUUG | | [1340-1358] |
| 261 | CCUGAACAGGCAUCUCACU | AGUGAGAUGCCUGUUCAGG | | [2225-2243] |
| 262 | AUUUUAAGAGGAAAGAAGA | UCUUCUUUCCUCUUAAAAU | | [140-158] |
| 263 | GACCUGGUGCUGCUGUUUA | UAAACAGCAGCACCAGGUC | | [2346-2364] |
| 264 | GCUUGGACUGACAUUUGAG | CUCAAAUGUCAGUCCAAGC | | [5340-5358] |
| 265 | AUGCCCUGCUCAUCAUCCA | UGGAUGAUGAGCAGGGCAU | | [3889-3907] |
| 266 | AUAUUGACCCAGACAAUUA | UAAUUGUCUGGGUCAAUAU | | [4995-5013] |
| 267 | AGAAGCUGUGAUGCUUAGA | UCUAAGCAUCACAGCUUCU | | [5410-5428] |
| 268 | CCUCAGGGAGAUCUACUCA | UGAGUAGAUCUCCCUGAGG | Rat | [4229-4247] |
| 269 | AGAUCUACUUCAUCUGGGU | ACCCAGAUGAAGUAGAUCU | Rat, Ms | [4441-4459] |
| 270 | CUUCCUCUCCAAGGACGAA | UUCGUCCUUGGAGAGGAAG | | [2822-2840] |
| 271 | CUCCCGGAGUAUACAGGAU | AUCCUGUAUACUCCGGGAG | | [1131-1149] |
| 272 | ACCAUGAUGCGAUCCUUCA | UGAAGGAUCGCAUCAUGGU | | [2847-2865] |
| 273 | CCUCCAUCCUCAAAGACCU | AGGUCUUUGAGGAUGGAGG | Rat, Ms | [4381-4399] |
| 274 | UCAUCCAGCUGCUGUCAGA | UCUGACAGCAGCUGGAUGA | | [2191-2209] |
| 275 | GAGAAAGGUGAGAGUGCAG | CUGCACUCUCACCUUUCUC | | [50-68] |
| 276 | GAGAAGUUCGACCUCAGGA | UCCUGAGGUCGAACUUCUC | Ms | [4569-4587] |
| 277 | GUACCCAGGAGGUGAAUGA | UCAUUCACCUCCUGGGUAC | | [1351-1369] |
| 278 | UCUGCUGCCUUCCCUUAGU | ACUAAGGGAAGGCAGCAGA | | [2017-2035] |
| 279 | UGGUCGCUGUUAUCAACAU | AUGUUGAUAACAGCGACCA | | [1834-1852] |
| 280 | AACUCAAAGAGAGCGUGAA | UUCACGCUCUCUUUGAGUU | | [2101-2119] |
| 281 | CACGCAGUCAAUGUCUACA | UGUAGACAUUGACUGCGUG | | [3636-3654] |
| 282 | UGCUGCCUUCCCUUAGUGA | UCACUAAGGGAAGGCAGCA | | [2019-2037] |
| 283 | GUAUGACAGGUGUGCUUCU | AGAAGCACACCUGUCAUAC | | [3769-3787] |
| 284 | AGGCUGUGACAAAGCAGCA | UGCUGCUUUGUCACAGCCU | | [2473-2491] |
| 285 | UGUUCUCCAAGAAGGAGAU | AUCUCCUUCUUGGAGAACA | | [1780-1798] |
| 286 | ACAUCCUGGUGGUCUUCAU | AUGAAGACCACCAGGAUGU | Ms | [2740-2758] |
| 287 | AGCACAGAAGCAAAGAUUU | AAAUCUUUGCUUCUGUGCU | | [125-143] |
| 288 | GCUCAAGAUCCCUAAGGAG | CUCCUUAGGGAUCUUGAGC | | [2324-2342] |
| 289 | AAGCUGUGAUGCUUAGAAC | GUUCUAAGCAUCACAGCUU | | [5412-5430] |
| 290 | ACCACUGAUGUGAUUUCUA | UAGAAAUCACAUCAGUGGU | | [6089-6107] |
| 291 | GAAUCCACCUUAGGCCUCA | UGAGGCCUAAGGUGGAUUC | | [4939-4957] |
| 292 | UCUUCAACUCCCUGCAGGA | UCCUGCAGGGAGUUGAAGA | | [4696-4714] |
| 293 | AAAAGAAACUCAAAGAGAG | CUCUCUUUGAGUUUCUUUU | | [2095-2113] |
| 294 | GAUGCUUAGAACCUGGACA | UGUCCAGGUUCUAAGCAUC | | [5419-5437] |
| 295 | AAGCAACCACUGAUGUGAU | AUCACAUCAGUGGUUGCUU | | [6084-6102] |
| 296 | UUGCCUCCAUCCUCAAAGA | UCUUUGAGGAUGGAGGCAA | Rat, Ms | [4378-4396] |
| 297 | GCUUCCUGCAGAAAACACU | AGUGUUUUCUGCAGGAAGC | | [1114-1132] |
| 298 | UGCAGAUCGUGCUUACUAC | GUAGUAAGCACGAUCUGCA | | [3371-3389] |
| 299 | UUGGGAGAAUAUUGACCCA | UGGGUCAAUAUUCUCCCAA | | [4987-5005] |
| 300 | AAUUGCAGUGCAUGAUGAU | AUCAUCAUGCACUGCAAUU | | [5048-5066] |
| 301 | CAGAAAACACUCCCGGAGU | ACUCCGGGAGUGUUUUCUG | | [1122-1140] |
| 302 | ACUUCAUGCACCACUAUGA | UCAUAGUGGUGCAUGAAGU | | [4822-4840] |
| 303 | AGCUGUCGAGUCUCGUUCA | UGAACGAGACUCGACAGCU | | [3084-3102] |
| 304 | AGGAUAAGUCCCGUCUAAU | AUUAGACGGGACUUAUCCU | | [2773-2791] |
| 305 | UGCUGUUUAGUUCUGAAGA | UCUUCAGAACUAAACAGCA | | [2356-2374] |
| 306 | AGGAAUGGGCUGUUCUCCA | UGGAGAACAGCCCAUUCCU | Ms | [1770-1788] |
| 307 | GAUUUUAAGAGGAAAGAAG | CUUCUUUCCUCUUAAAAUC | | [139-157] |
| 308 | CCACAAAAUCGAUGCGUUG | CAACGCAUCGAUUUUGUGG | | [5310-5328] |
| 309 | CCAUGCUAUACAUCUGCGA | UCGCAGAUGUAUAGCAUGG | | [4591-4609] |
| 310 | GAAGCAACCACUGAUGUGA | UCACAUCAGUGGUUGCUUC | | [6083-6101] |
| 311 | AGUUGGAGGACAACAUAGU | ACUAUGUUGUCCUCCAACU | | [1402-1420] |
| 312 | ACAACUACUGGAUUCGGGA | UCCCGAAUCCAGUAGUUGU | Rat | [1315-1333] |
| 313 | AAGAGGUGGAGGAGAACGA | UCGUUCUCCUCCACCUCUU | | [4504-4522] |
| 314 | GAUGCGUUGCUUUGGUAUU | AAUACCAAAGCAACGCAUC | | [5320-5338] |
| 315 | AACACUCCCGGAGUAUACA | UGUAUACUCCGGGAGUGUU | | [1127-1145] |
| 316 | CCACUGAUGUGAUUUCUAC | GUAGAAAUCACAUCAGUGG | | [6090-6108] |
| 317 | CAAGACCAGAGGCACUGAU | AUCAGUGCCUCUGGUCUUG | | [214-232] |
| 318 | GCAAGAGAAGAUGCAGCGA | UCGCUGCAUCUUCUCUUGC | | [3248-3266] |
| 319 | CAGUACCCAGGAGGUGAAU | AUUCACCUCCUGGGUACUG | | [1349-1367] |
| 320 | AGACAGUCCCUGCUUGUGA | UCACAAGCAGGGACUGUCU | | [5224-5242] |
| 321 | AGAUCCCUAAGGAGUAUGA | UCAUACUCCUUAGGGAUCU | | [2329-2347] |
| 322 | CUCAGGGAGAUCUACUCAU | AUGAGUAGAUCUCCCUGAG | | [4230-4248] |
| 323 | CCCUGCUUGUGACAAGUGU | ACACUUGUCACAAGCAGGG | | [5231-5249] |
| 324 | GUGUCAGCAACUUUGAGGA | UCCUCAAAGUUGCUGACAC | | [5524-5542] |
| 325 | UCCAGUUUUGGUCCUACUA | UAGUAGGACCAAAACUGGA | | [6113-6131] |
| 326 | CUGACAUCAUCCAAGAGGU | ACCUCUUGGAUGAUGUCAG | | [4492-4510] |
| 327 | UCAUGCACCACUAUGAGAA | UUCUCAUAGUGGUGCAUGA | | [4825-4843] |
| 328 | AUUUUACAUCGAGGUAUAA | UUAUACCUCGAUGUAAAAU | | [5946-5964] |
| 329 | CGCUAUGUGCCUUUUGAUG | CAUCAAAAGGCACAUAGCG | | [3549-3567] |
| 330 | CUGAGAAGUUCGACCUCAG | CUGAGGUCGAACUUCUCAG | Ms | [4567-4585] |
| 331 | ACCCAGGUCAAGACAUAGA | UCUAUGUCUUGACCUGGGU | | [5993-6011] |
| 332 | CCUGGUGCUGCUGUUUAGU | ACUAAACAGCAGCACCAGG | | [2348-2366] |
| 333 | GAAAUGGUAUUAGAGAUAU | AUAUCUCUAAUACCAUUUC | | [3049-3067] |
| 334 | UGGACAUCCUGGUGGUCUU | AAGACCACCAGGAUGUCCA | Ms | [2737-2755] |
| 335 | CCCUAGCUCCUAGAAGCAA | UUGCUUCUAGGAGCUAGGG | | [6071-6089] |
| 336 | ACUAUGUUCUACAAUUGCA | UGCAAUUGUAGAACAUAGU | | [5036-5054] |
| 337 | CCAAGGAACUGGAGUGAUC | GAUCACUCCAGUUCCUUGG | | [1560-1578] |
| 338 | AAUUCUCCCGUACAGACUA | UAGUCUGUACGGGAGAAUU | | [1459-1477] |
| 339 | AGGUGCUGGACAUCAACCA | UGGUUGAUGUCCAGCACCU | Ms | [2536-2554] |
| 340 | UGACAGGUGUGCUUCUGCU | AGCAGAAGCACACCUGUCA | | [3772-3790] |
| 341 | GAAAACACUCCCGGAGUAU | AUACUCCGGGAGUGUUUUC | | [1124-1142] |
| 342 | GUGAUUUCUACCAAAUCCA | UGGAUUUGGUAGAAAUCAC | | [6098-6116] |
| 343 | UGUACAUGUUUUACCACCA | UGGUGGUAAAACAUGUACA | | [5975-5993] |
| 344 | UGCCCUGCUCAUCAUCCAU | AUGGAUGAUGAGCAGGGCA | | [3890-3908] |
| 345 | ACCAAGGACCCAGAGAGAA | UUCUCUCUGGGUCCUUGGU | | [36-54] |
| 346 | ACGGCUGGUUUAACAACCU | AGGUUGUUAAACCAGCCGU | | [319-337] |
| 347 | AGGAGCAGUCCCAUCAUCA | UGAUGAUGGGACUGCUCCU | | [2175-2193] |
| 348 | UGACUGUGCUUGACUUCUU | AAGAAGUCAAGCACAGUCA | | [1957-1975] |
| 349 | AGGUCAAGACAUAGAAUGU | ACAUUCUAUGUCUUGACCU | | [5997-6015] |
| 350 | GGAAUGACCAAGAAUGUAG | CUACAUUCUUGGUCAUUCC | | [4764-4782] |
| 351 | AAUAUUGGGAGAAUAUUGA | UCAAUAUUCUCCCAAUAUU | | [4983-5001] |
| 352 | AGACCUGGUCUUCAAGUCA | UGACUUGAAGACCAGGUCU | | [4394-4412] |
| 353 | CUGGAGUGAUCUCAACCCU | AGGGUUGAGAUCACUCCAG | | [1568-1586] |
| 354 | AGUUCUUCUCUACCAUGGU | ACCAUGGUAGAGAAGAACU | | [1204-1222] |
| 355 | CUGCCUUCUCUUCUGUGCA | UGCACAGAAGAGAAGGCAG | | [4883-4901] |
| 356 | GGACGAAUUCUUCACCAUG | CAUGGUGAAGAAUUCGUCC | | [2834-2852] |
| 357 | CGUUGCUUUGGUAUUUGCU | AGCAAAUACCAAAGCAACG | | [5324-5342] |
| 358 | GAAGGUCCUGAACAAGGGU | ACCCUUGUUCAGGACCUUC | | [1265-1283] |
| 359 | GUUGAAGAUCUGAGGGAUU | AAUCCCUCAGAUCUUCAAC | | [1422-1440] |
| 360 | GCCCUGAUAUUCUUGAUGU | ACAUCAAGAAUAUCAGGGC | | [5572-5590] |
| 361 | CGGCUGGUUUAACAACCUG | CAGGUUGUUAAACCAGCCG | | [320-338] |
| 362 | AGAUAUCUUUAAACAAAAC | GUUUUGUUUAAAGAUAUCU | | [3062-3080] |
| 363 | GCGAGAGACUUUCCUCAAC | GUUGAGGAAAGUCUCUCGC | | [3530-3548] |
| 364 | UCUUGAUGUCACCAGUGAU | AUCACUGGUGACAUCAAGA | | [5582-5600] |
| 365 | AAUGGGCUGUUCUCCAAGA | UCUUGGAGAACAGCCCAUU | | [1773-1791] |
| 366 | GCUCCUAGAAGCAACCACU | AGUGGUUGCUUCUAGGAGC | | [6076-6094] |
| 367 | CAGGAUACCGUCCUUUCCU | AGGAAAGGACGGUAUCCUG | | [1144-1162] |
| 368 | AAGAUUUUAAGAGGAAAGA | UCUUUCCUCUUAAAAUCUU | | [137-155] |
| 369 | AGUACAAGCGCUUCGUGGA | UCCACGAAGCGCUUGUACU | | [3292-3310] |
| 370 | GCAUCCUGGAGAUCUUCUU | AAGAAGAUCUCCAGGAUGC | | [2500-2518] |
| 371 | GACAUGGGAGGAUUUUCAC | GUGAAAAUCCUCCCAUGUC | | [2960-2978] |
| 372 | GAAGAUCUGAGGGAUUACU | AGUAAUCCCUCAGAUCUUC | | [1425-1443] |
| 373 | CAGUGCAGAAGCUGUGAUG | CAUCACAGCUUCUGCACUG | | [5404-5422] |
| 374 | CAAGAAGGAGAUUGAAGAC | GUCUUCAAUCUCCUUCUUG | | [1787-1805] |
| 375 | CAGUCCCUGCUUGUGACAA | UUGUCACAAGCAGGGACUG | | [5227-5245] |
| 376 | UUCACUUCAUGCUGCGGGA | UCCCGCAGCAUGAAGUGAA | | [2974-2992] |
| 377 | UGCAGAAGCUGUGAUGCUU | AAGCAUCACAGCUUCUGCA | | [5407-5425] |
| 378 | UGAACCCAACACCACCCUA | UAGGGUGGUGUUGGGUUCA | | [163-181] |
| 379 | GAUAAAGACCACUCCUCAC | GUGAGGAGUGGUCUUUAUC | | [5162-5180] |
| 380 | AGAUGAGCGAGAAGGAGCU | AGCUCCUUCUCGCUCAUCU | | [2446-2464] |
| 381 | ACUCCCGGAGUAUACAGGA | UCCUGUAUACUCCGGGAGU | | [1130-1148] |
| 382 | AAGUAGAGCACAGAAGCAA | UUGCUUCUGUGCUCUACUU | | [119-137] |
| 383 | GAUAUCUUUAAACAAAACA | UGUUUUGUUUAAAGAUAUC | | [3063-3081] |
| 384 | GGUGCUGCUGUUUAGUUCU | AGAACUAAACAGCAGCACC | | [2351-2369] |
| 385 | CCUCAUAACCUUCCUGCGA | UCGCAGGAAGGUUAUGAGG | | [3515-3533] |
| 386 | CUAUGUUCUACAAUUGCAG | CUGCAAUUGUAGAACAUAG | | [5037-5055] |
| 387 | CAAAGACCUGGUCUUCAAG | CUUGAAGACCAGGUCUUUG | | [4391-4409] |
| 388 | CUUUUGAUGCCGCAGUGGA | UCCACUGCGGCAUCAAAAG | | [3559-3577] |
| 389 | GUCCUGAACAGGCAUCUCA | UGAGAUGCCUGUUCAGGAC | | [2223-2241] |
| 390 | GUUUGCAGAUCGUGCUUAC | GUAAGCACGAUCUGCAAAC | | [3368-3386] |
| 391 | AUACAAAUGAGAAAAGGCA | UGCCUUUUCUCAUUUGUAU | | [5013-5031] |
| 392 | CAAGGACCCAGAGAGAAAG | CUUUCUCUCUGGGUCCUUG | | [38-56] |
| 393 | CCUGGUGUCUGUGCACAUU | AAUGUGCACAGACACCAGG | | [4532-4550] |
| 394 | CUGUUCUCCAAGAAGGAGA | UCUCCUUCUUGGAGAACAG | | [1779-1797] |
| 395 | ACAUAGUGGUUGAAGAUCU | AGAUCUUCAACCACUAUGU | | [1414-1432] |
| 396 | AGACUUCCUGCCCUUAGCA | UGCUAAGGGCAGGAAGUCU | | [5112-5130] |
| 397 | UCCGAGAGUUCCUGGACAU | AUGUCCAGGAACUCUCGGA | | [2725-2743] |
| 398 | GAUGAGCGAGAAGGAGCUA | UAGCUCCUUCUCGCUCAUC | | [2447-2465] |
| 399 | GCCUUUGUGCAGCAGCUAU | AUAGCUGCUGCACAAAGGC | | [2385-2403] |
| 400 | GACAUCCCAAGGAACUGGA | UCCAGUUCCUUGGGAUGUC | | [1554-1572] |
| 401 | UUAGAGAUAUCUUUAAACA | UGUUUAAAGAUAUCUCUAA | | [3058-3076] |
| 402 | CCAAAGUGUCAGCAACUUU | AAAGUUGCUGACACUUUGG | | [5519-5537] |
| 403 | UGGUUGAAGAUCUGAGGGA | UCCCUCAGAUCUUCAACCA | | [1420-1438] |
| 404 | CCUUAGUGAGUCUGCUUCU | AGAAGCAGACUCACUAAGG | | [2029-2047] |
| 405 | CACCCAGGUCAAGACAUAG | CUAUGUCUUGACCUGGGUG | | [5992-6010] |
| 406 | AGGCAAGAAGUAGAGCACA | UGUGCUCUACUUCUUGCCU | | [112-130] |
| 407 | AAGCUACAAAAGAAACUCA | UGAGUUUCUUUUGUAGCUU | | [2088-2106] |
| 408 | UCUGAGCAGUUCUUCUCUA | UAGAGAAGAACUGCUCAGA | | [1197-1215] |
| 409 | CUGGUCACCUUACGCAAGA | UCUUGCGUAAGGUGACCAG | | [5836-5854] |
| 410 | ACUUCCAGAAAGUGCUGAA | UUCAGCACUUUCUGGAAGU | | [4615-4633] |
| 411 | GGCUGACAUCAUCCAAGAG | CUCUUGGAUGAUGUCAGCC | | [4490-4508] |
| 412 | AUUUCCGGAAGGUCCUGAA | UUCAGGACCUUCCGGAAAU | | [1258-1276] |
| 413 | GCCUCUGAGCAGUUCUUCU | AGAAGAACUGCUCAGAGGC | | [1194-1212] |
| 414 | AGUGGUUGAAGAUCUGAGG | CCUCAGAUCUUCAACCACU | | [1418-1436] |
| 415 | AAGGACGAAUUCUUCACCA | UGGUGAAGAAUUCGUCCUU | | [2832-2850] |
| 416 | AUGAGCUGCUGCUGGGAAU | AUUCCCAGCAGCAGCUCAU | | [1366-1384] |
| 417 | GAGCUGACAUGGGAGGAUU | AAUCCUCCCAUGUCAGCUC | | [2955-2973] |
| 418 | GUCAAUGUCUACAUCUUCU | AGAAGAUGUAGACAUUGAC | | [3642-3660] |
| 419 | AGCGGCACUUCCAGAAAGU | ACUUUCUGGAAGUGCCGCU | | [4609-4627] |
| 420 | GAGUUGGAGGACAACAUAG | CUAUGUUGUCCUCCAACUC | | [1401-1419] |
| 421 | CAUGCACCACUAUGAGAAC | GUUCUCAUAGUGGUGCAUG | | [4826-4844] |
| 422 | AAGUCCCGUCUAAUGUUUA | UAAACAUUAGACGGGACUU | | [2778-2796] |
| 423 | UCUAAUGUUUACCAUGUAU | AUACAUGGUAAACAUUAGA | | [2786-2804] |
| 424 | AUCUUUAAACAAAACAUCA | UGAUGUUUUGUUUAAAGAU | | [3066-3084] |
| 425 | UUCUCUUCUGUGCACCUAA | UUAGGUGCACAGAAGAGAA | | [4888-4906] |
| 426 | GAUUCCAGGACAAGGAGGA | UCCUCCUUGUCCUGGAAUC | | [2938-2956] |
| 427 | UGGAGGACAACAUAGUGGU | ACCACUAUGUUGUCCUCCA | | [1405-1423] |
| 428 | AGCUAUUUAGGAAGGCUGU | ACAGCCUUCCUAAAUAGCU | | [2461-2479] |
| 429 | AUCUGAACAGUACCCAGGA | UCCUGGGUACUGUUCAGAU | | [1342-1360] |
| 430 | AAGGUACAAUAGUGCUUGU | ACAAGCACUAUUGUACCUU | | [5259-5277] |
| 431 | CACCUACCAGAACAUCGCU | AGCGAUGUUCUGGUAGGUG | | [1076-1094] |
| 432 | UGGUUUGAGAACACCAGGA | UCCUGGUGUUCUCAAACCA | | [1755-1773] |
| 433 | GUUUAUCAACGGCACCAUU | AAUGGUGCCGUUGAUAAAC | | [5080-5098] |
| 434 | UGUCCUUCCGAGAGUUCCU | AGGAACUCUCGGAAGGACA | | [2719-2737] |
| 435 | CUUUAACUUUUCUUUUCCU | AGGAAAAGAAAAGUUAAAG | | [5887-5905] |
| 436 | GAAUGUAGAGAAGGCCUGU | ACAGGCCUUCUCUACAUUC | | [4775-4793] |
| 437 | AAUAGUGCUUGUCUCCGAU | AUCGGAGACAAGCACUAUU | | [5266-5284] |
| 438 | AGAUCUUCUUCAGACACCU | AGGUGUCUGAAGAAGAUCU | | [2509-2527] |
| 439 | CUUUGCCUCCAUCCUCAAA | UUUGAGGAUGGAGGCAAAG | Rat, Ms | [4376-4394] |
| 440 | GCAGGAGACUAUGUUCUAC | GUAGAACAUAGUCUCCUGC | | [5029-5047] |
| 441 | CGUCUUUGUGAAUGAUGGG | CCCAUCAUUCACAAAGACG | | [3701-3719] |
| 442 | AGUGAGGGUGAAGGCAAGA | UCUUGCCUUCACCCUCACU | | [101-119] |
| 443 | UGCUGAACCGGAGUCUGUU | AACAGACUCCGGUUCAGCA | | [4627-4645] |
| 444 | CAGGUAUGACAGGUGUGCU | AGCACACCUGUCAUACCUG | | [3766-3784] |
| 445 | CGGAGUAUACAGGAUACCG | CGGUAUCCUGUAUACUCCG | | [1135-1153] |
| 446 | CAUCGAGAUCUCCAACAAC | GUUGUUGGAGAUCUCGAUG | Rat | [2864-2882] |
| 447 | CAAUAUUGGGAGAAUAUUG | CAAUAUUCUCCCAAUAUUG | | [4982-5000] |
| 448 | GAUCCUUCAUCGAGAUCUC | GAGAUCUCGAUGAAGGAUC | | [2857-2875] |
| 449 | GUGCUUGACUUCUUUGAAG | CUUCAAAGAAGUCAAGCAC | | [1962-1980] |
| 450 | UCCUGAACAAGGGUUUUCA | UGAAAACCCUUGUUCAGGA | | [1270-1288] |
| 451 | CCCAAGGAACUGGAGUGAU | AUCACUCCAGUUCCUUGGG | | [1559-1577] |
| 452 | UGUUUAGUUCUGAAGAGGA | UCCUCUUCAGAACUAAACA | | [2359-2377] |
| 453 | AGGUGAAUGAGCUGCUGCU | AGCAGCAGCUCAUUCACCU | | [1360-1378] |
| 454 | CAUACUCAGCUCUUUAACU | AGUUAAAGAGCUGAGUAUG | | [5876-5894] |
| 455 | CAAAAGGAGCAGCCCUACU | AGUAGGGCUGCUCCUUUUG | | [5483-5501] |
| 456 | GCUGCUCAAGAUCCCUAAG | CUUAGGGAUCUUGAGCAGC | | [2321-2339] |
| 457 | CCAGACAAUUAUACAAAUG | CAUUUGUAUAAUUGUCUGG | | [5003-5021] |
| 458 | GCUCUUUCCUAAAAAGCUG | CAGCUUUUUAGGAAAGAGC | | [5916-5934] |
| 459 | UUGAGUACAAGUCAGGACA | UGUCCUGACUUGUACUCAA | | [4087-4105] |
| 460 | AUUUAGGAAGGCUGUGACA | UGUCACAGCCUUCCUAAAU | | [2465-2483] |
| 461 | AACACCACCCUAAACCACA | UGUGGUUUAGGGUGGUGUU | | [170-188] |
| 462 | CUCUUUCCUAAAAAGCUGA | UCAGCUUUUUAGGAAAGAG | | [5917-5935] |
| 463 | GUUGGAGGACAACAUAGUG | CACUAUGUUGUCCUCCAAC | | [1403-1421] |
| 464 | AACUUUUCUUUUCCUUUUU | AAAAAGGAAAAGAAAAGUU | | [5891-5909] |
| 465 | CAAAGAGAGCGUGAAGAAG | CUUCUUCACGCUCUCUUUG | | [2105-2123] |
| 466 | CUUCAUGCACCACUAUGAG | CUCAUAGUGGUGCAUGAAG | | [4823-4841] |
| 467 | AUGGUAUUAGAGAUAUCUU | AAGAUAUCUCUAAUACCAU | | [3052-3070] |
| 468 | AGGUCAACCUCAUCCUGUC | GACAGGAUGAGGUUGACCU | Rat | [2278-2296] |
| 469 | AUCUCAACCCUAAUGUGGA | UCCACAUUAGGGUUGAGAU | | [1576-1594] |
| 470 | CCAGGAAACAAAGAUGCCU | AGGCAUCUUUGUUUCCUGG | | [5548-5566] |
| 471 | CGAAUUCUUCACCAUGAUG | CAUCAUGGUGAAGAAUUCG | | [2837-2855] |
| 472 | UGGAGUCUAUGUUCCGGGA | UCCCGGAACAUAGACUCCA | | [2914-2932] |
| 473 | AGCAACCACUGAUGUGAUU | AAUCACAUCAGUGGUUGCU | | [6085-6103] |
| 474 | GUUGCUUUGGUAUUUGCUU | AAGCAAAUACCAAAGCAAC | | [5325-5343] |
| 475 | ACCCAGAGAGAAAGGUGAG | CUCACCUUUCUCUCUGGGU | | [43-61] |
| 476 | AGGACCACCAUGCUAUACA | UGUAUAGCAUGGUGGUCCU | | [4584-4602] |
| 477 | UGUAAGAAGAUCUACUUCA | UGAAGUAGAUCUUCUUACA | Rat, Ms | [4434-4452] |
| 478 | AGAAGCUACAAAAGAAACU | AGUUUCUUUUGUAGCUUCU | | [2086-2104] |
| 479 | CUCUCUGCAUCUACUGGAG | CUCCAGUAGAUGCAGAGAG | | [5630-5648] |
| 480 | GAUAUUCAGUACUCGAUCU | AGAUCGAGUACUGAAUAUC | | [5693-5711] |
| 481 | CAUCUGAACCCAACACCAC | GUGGUGUUGGGUUCAGAUG | | [159-177] |
| 482 | UGGUGGUCUUCAUGAAAGG | CCUUUCAUGAAGACCACCA | Ms | [2746-2764] |
| 483 | GAUACCGUCCUUUCCUAGA | UCUAGGAAAGGACGGUAUC | | [1147-1165] |
| 484 | AGCUCUUUAACUUUUCUUU | AAAGAAAAGUUAAAGAGCU | | [5883-5901] |
| 485 | UUUUCUUUUCCUUUUUCAU | AUGAAAAAGGAAAAGAAAA | | [5894-5912] |
| 486 | UCCAAAAGGAGCAGCCCUA | UAGGGCUGCUCCUUUUGGA | | [5481-5499] |
| 487 | GUGUUUGCAGAUCGUGCUU | AAGCACGAUCUGCAAACAC | | [3366-3384] |
| 488 | CAGAAUCCACCUUAGGCCU | AGGCCUAAGGUGGAUUCUG | | [4937-4955] |
| 489 | GAUCUUCUUCAGACACCUU | AAGGUGUCUGAAGAAGAUC | | [2510-2528] |
| 490 | CACCACUAUGAGAACUUCU | AGAAGUUCUCAUAGUGGUG | | [4830-4848] |
| 491 | GAGUAUACAGGAUACCGUC | GACGGUAUCCUGUAUACUC | | [1137-1155] |
| 492 | CCCUAAGGAGUAUGACCUG | CAGGUCAUACUCCUUAGGG | | [2333-2351] |
| 493 | UCAUGUUCUCUUAUAUCUU | AAGAUAUAAGAGAACAUGA | | [3478-3496] |
| 494 | GUGACAAGUGUGGAGGUGA | UCACCUCCACACUUGUCAC | | [5239-5257] |
| 495 | AUCUCUCCAUCAGAAUCCA | UGGAUUCUGAUGGAGAGAU | | [4927-4945] |
| 496 | UCCUAGAAGCAACCACUGA | UCAGUGGUUGCUUCUAGGA | | [6078-6096] |
| 497 | GGAAAGAAGACAUCUGAAC | GUUCAGAUGUCUUCUUUCC | | [149-167] |
| 498 | GAGGUGGUGGAGUCUAUGU | ACAUAGACUCCACCACCUC | | [2907-2925] |
| 499 | GGCACUUCCAGAAAGUGCU | AGCACUUUCUGGAAGUGCC | | [4612-4630] |
| 500 | GAAGGCUGUGACAAAGCAG | CUGCUUUGUCACAGCCUUC | | [2471-2489] |

TABLE A11
NOXO1 19-mer oligomer pairs (NADPH oxidase organizer 1)

| | | | | Human- | Human- | Human- |
|---|---|---|---|---|---|---|
| | | | | 34222190 | 41281827 | 41281810 |
| | | | Other | ORF: | ORF: | ORF: |
| No. | Sense siRNA | AntiSense siRNA | Sp | 261-1373 | 5-1135 | 5-1120 |
| 1 | CGACGAUCACUGGCUUCUU | AAGAAGCCAGUGAUCGUCG | | [595-613 | [357-375 | [342-360 |
| 2 | GCGUGUUGGAAACGUCAGA | UCUGACGUUUCCAACACGC | | [1036-1054 | [798-816 | [783-801 |
| 3 | CCAAACGUUUGCCUUCUCU | AGAGAAGGCAAACGUUUGG | | [323-341 | [67-85 | [67-85 |
| 4 | CAGAUCAAGAGGCUCCAAA | UUUGGAGCCUCUUGAUCUG | | [309-327 | [53-71 | [53-71 |
| 5 | GCAGAUCAAGAGGCUCCAA | UUGGAGCCUCUUGAUCUGC | | [308-326 | [52-70 | [52-70 |
| 6 | UGCUGUUCCUCUGAGUAAA | UUUACUCAGAGGAACAGCA | | [1475-1493] | — | — |
| 7 | GCUGUUGGAAACCUAUUCU | AGAAUAGGUUUCCAACAGC | | [536-554 | [298-316 | [283-301 |
| 8 | GGAGUUGGGACGAAUUCAG | CUGAAUUCGUCCCAACUCC | | [379-397 | [123-141 | [123-141 |
| 9 | UCCAAACGUUUGCCUUCUC | GAGAAGGCAAACGUUUGGA | | [322-340 | [66-84 | [66-84 |
| 10 | CUCCAAACGUUUGCCUUCU | AGAAGGCAAACGUUUGGAG | | [321-339 | [65-83 | [65-83 |
| 11 | AGCUGUUGGAAACCUAUUC | GAAUAGGUUUCCAACAGCU | | [535-553 | [297-315 | [282-300 |
| 12 | CGAAUUCAGGCAGCUCAAG | CUUGAGCUGCCUGAAUUCG | | [389-407 | [133-151 | [133-151 |
| 13 | ACGAAUUCAGGCAGCUCAA | UUGAGCUGCCUGAAUUCGU | | [388-406 | [132-150 | [132-150 |
| 14 | CAAGAAAGCUGCAAAGGAC | GUCCUUUGCAGCUUUCUUG | | [87-105] | — | — |
| 15 | CGUGUUGGAAACGUCAGAC | GUCUGACGUUUCCAACACG | | [1037-1055 | [799-817 | [784-802 |
| 16 | CUGUUGGAAACCUAUUCUC | GAGAAUAGGUUUCCAACAG | | [537-555 | [299-317 | [284-302 |
| 17 | GACGAAUUCAGGCAGCUCA | UGAGCUGCCUGAAUUCGUC | | [387-405 | [131-149 | [131-149 |
| 18 | GCUGUUCCUCUGAGUAAAG | CUUUACUCAGAGGAACAGC | | [1476-1494] | — | — |
| 19 | UGGUGCAGAUCAAGAGGCU | AGCCUCUUGAUCUGCACCA | | [304-322 | [48-66 | [48-66 |
| 20 | UUGAGGUGUUGCCAGGAUU | AAUCCUGGCAACACCUCAA | | [140-158] | — | — |
| 21 | GCAGCUGUUGGAAACCUAU | AUAGGUUUCCAACAGCUGC | | [533-551 | [295-313 | [280-298 |
| 22 | CCUCGCCCAUUUCAGGAAU | AUUCCUGAAAUGGGCGAGG | | [220-238] | — | — |
| 23 | GCCAUUUGCUGUUCCUCUG | CAGAGGAACAGCAAAUGGC | | [1469-1487] | — | — |
| 24 | UCAAGACCCUCAAGGAGAC | GUCUCCUUGAGGGUCUUGA | | [403-421 | [152-168] | [152-168] |
| | | | | | (17/17) | (17/17) |
| 25 | CCAUUUGCUGUUCCUCUGA | UCAGAGGAACAGCAAAUGG | | [1470-1488] | — | — |
| 26 | UCGCCCAUUUCAGGAAUCU | AGAUUCCUGAAAUGGGCGA | | [222-240] | — | — |
| 27 | CUCGCCCAUUUCAGGAAUC | GAUUCCUGAAAUGGGCGAG | | [221-239] | — | — |
| 28 | AGCUUCUCGAUGCACCACU | AGUGGUGCAUCGAGAAGCU | | [472-490 | [242-252] | [219-237 |
| | | | | | (11/11) | |
| | | | | | ORF | |
| 29 | AUACCCAGUUUCAGUGCAA | UUGCACUGAAACUGGGUAU | | [275-293 | [19-37 | [19-37 |
| 30 | CCGAUACCCAGUUUCAGUG | CACUGAAACUGGGUAUCGG | | [272-290 | [16-34 | [16-34 |
| 31 | AGGAAUCUGCAGCCUGGAA | UUCCAGGCUGCAGAUUCCU | | [233-251] | — | — |
| 32 | CGCCCAUUUCAGGAAUCUG | CAGAUUCCUGAAAUGGGCG | | [223-241] | — | — |
| 33 | AGACCCUCAAGGAGACCUU | AAGGUCUCCUUGAGGGUCU | | [406-424 | [153-171 | [153-171 |
| 34 | CCAUUUCAGGAAUCUGCAG | CUGCAGAUUCCUGAAAUGG | | [226-244] | — | — |
| 35 | CCCAUUUCAGGAAUCUGCA | UGCAGAUUCCUGAAAUGGG | | [225-243] | — | — |
| 36 | ACAGCAAGAAAGCUGCAAA | UUUGCAGCUUUCUUGCUGU | | [83-101] | — | — |
| 37 | CCCGAUACCCAGUUUCAGU | ACUGAAACUGGGUAUCGGG | | [271-289 | [15-33 | [15-33 |
| 38 | UGGAAACCUAUUCUCGGAG | CUCCGAGAAUAGGUUUCCA | | [541-559 | [303-321 | [288-306 |
| 39 | UCCUCGCCCAUUUCAGGAA | UUCCUGAAAUGGGCGAGGA | | [219-237] | — | — |
| 40 | CUUGAGGUGUUGCCAGGAU | AUCCUGGCAACACCUCAAG | | [139-157] | — | — |
| 41 | GCCCAUUUCAGGAAUCUGC | GCAGAUUCCUGAAAUGGGC | | [224-242] | — | — |
| 42 | CAGCAAGAAAGCUGCAAAG | CUUUGCAGCUUUCUUGCUG | | [84-102] | — | — |
| 43 | GGCUCCAAACGUUUGCCUU | AAGGCAAACGUUUGGAGCC | | [319-337 | [63-81 | [63-81 |
| 44 | UGCCAGGAUUCUAUCCCAG | CUGGGAUAGAAUCCUGGCA | | [149-167] | — | — |
| 45 | GGAAACCUAUUCUCGGAGG | CCUCCGAGAAUAGGUUUCC | | [542-560 | [304-322 | [289-307 |
| 46 | CAGCUGUUGGAAACCUAUU | AAUAGGUUUCCAACAGCUG | | [534-552 | [296-314 | [281-299 |
| 47 | GACAGCAAGAAAGCUGCAA | UUGCAGCUUUCUUGCUGUC | | [82-100] | — | — |
| 48 | UGCAGCUGUUGGAAACCUA | UAGGUUUCCAACAGCUGCA | | [532-550 | [294-312 | [279-297 |
| 49 | AGGCUCCAAACGUUUGCCU | AGGCAAACGUUUGGAGCCU | | [318-336 | [62-80 | [62-80 |
| 50 | GGACAGCAAGAAAGCUGCA | UGCAGCUUUCUUGCUGUCC | | [81-99] | — | — |
| 51 | CUGCAGCUGUUGGAAACCU | AGGUUUCCAACAGCUGCAG | | [531-549 | [293-311 | [278-296 |
| 52 | GGCCAUUUGCUGUUCCUCU | AGAGGAACAGCAAAUGGCC | | [1468-1486] | — | — |

TABLE A12
NOXO1 - 19-mer oligomer pairs NADPH oxidase organizer 1

| | | | | Human- | Human- | Human- |
|---|---|---|---|---|---|---|
| | | | Other | 34222190 | 41281827 | 41281810 |
| No. | Sense siRNA | AntiSense siRNA | Sp | ORF: 261-1373 | ORF: 5-1135 | ORF: 5-1120 |
| 1 | GCAGGAGUUGGGACGAAUU | AAUUCGUCCCAACUCCUGC | | [376-394] | [120-138] | [120-138] |
| 2 | CAAGAGGCUCCAAACGUUU | AAACGUUUGGAGCCUCUUG | | [314-332] | [58-76] | [58-76] |
| 3 | UCAAGAGGCUCCAAACGUU | AACGUUUGGAGCCUCUUGA | | [313-331] | [57-75] | [57-75] |
| 4 | AGAGGAGCAGCCUCUUUCU | AGAAAGAGGCUGCUCCUCU | | [680-698] | [442-460] | [427-445] |
| 5 | AGGAGUUGGGACGAAUUCA | UGAAUUCGUCCCAACUCCU | | [378-396] | [122-140] | [122-140] |
| 6 | UUGGAAACCUAUUCUCGGA | UCCGAGAAUAGGUUUCCAA | | [540-558] | [302-320] | [287-305] |
| 7 | GAAUUCAGGCAGCUCAAGA | UCUUGAGCUGCCUGAAUUC | | [390-408] | [134-152] | [134-152] |
| 8 | AGAUCAAGAGGCUCCAAAC | GUUUGGAGCCUCUUGAUCU | | [310-328] | [54-72] | [54-72] |
| 9 | GCGUUCUCCCAAAGCUUCU | AGAAGCUUUGGGAGAACGC | | [460-478] | [207-225] | [207-225] |
| 10 | GAUACCCAGUUUCAGUGCA | UGCACUGAAACUGGGUAUC | | [274-292] | [18-36] | [18-36] |
| 11 | UGCAGAUCAAGAGGCUCCA | UGGAGCCUCUUGAUCUGCA | | [307-325] | [51-69] | [51-69] |
| 12 | GUUGGAAACCUAUUCUCGG | CCGAGAAUAGGUUUCCAAC | | [539-557] | [301-319] | [286-304] |
| 13 | AAACGUUUGCCUUCUCUGU | ACAGAGAAGGCAAACGUUU | | [325-343] | [69-87] | [69-87] |
| 14 | GAGUUGGGACGAAUUCAGG | CCUGAAUUCGUCCCAACUC | | [380-398] | [124-142] | [124-142] |
| 15 | CAGGAGUUGGGACGAAUUC | GAAUUCGUCCCAACUCCUG | | [377-395] | [121-139] | [121-139] |
| 16 | GAUCAAGAGGCUCCAAACG | CGUUUGGAGCCUCUUGAUC | | [311-329] | [55-73] | [55-73] |
| 17 | GUUGGGACGAAUUCAGGCA | UGCCUGAAUUCGUCCCAAC | | [382-400] | [126-144] | [126-144] |
| 18 | UCUCCAUCCACAGUCUGGA | UCCAGACUGUGGAUGGAGA | | [715-733] | [477-495] | [462-480] |
| 19 | UACCCAGUUUCAGUGCAAG | CUUGCACUGAAACUGGGUA | | [276-294] | [20-38] | [20-38] |
| 20 | CAAACGUUUGCCUUCUCUG | CAGAGAAGGCAAACGUUUG | | [324-342] | [68-86] | [68-86] |
| 21 | ACCCAGUUUCAGUGCAAGG | CCUUGCACUGAAACUGGGU | | [277-295] | [21-39] | [21-39] |
| 22 | AUAGGCCUUUUCAGGCGCA | UGCGCCUGAAAAGGCCUAU | | [784-802] | [546-564] | [531-549] |
| 23 | CGUUCUCCCAAAGCUUCUC | GAGAAGCUUUGGGAGAACG | | [461-479] | [208-226] | [208-226] |
| 24 | AAGAGGCUCCAAACGUUUG | CAAACGUUUGGAGCCUCUU | | [315-333] | [59-77] | [59-77] |
| 25 | UGUUGGAAACCUAUUCUCG | CGAGAAUAGGUUUCCAACA | | [538-556] | [300-318] | [285-303] |
| 26 | CGAUACCCAGUUUCAGUGC | GCACUGAAACUGGGUAUCG | | [273-291] | [17-35] | [17-35] |
| 27 | AGAGGCUCCAAACGUUUGC | GCAAACGUUUGGAGCCUCU | | [316-334] | [60-78] | [60-78] |
| 28 | AUCAAGAGGCUCCAAACGU | ACGUUUGGAGCCUCUUGAU | | [312-330] | [56-74] | [56-74] |
| 29 | AAACCUAUUCUCGGAGGCU | AGCCUCCGAGAAUAGGUUU | | [544-562] | [306-324] | [291-309] |
| 30 | GCUCCAAACGUUUGCCUUC | GAAGGCAAACGUUUGGAGC | | [320-338] | [64-82] | [64-82] |
| 31 | UGUUGGAAACGUCAGACCG | CGGUCUGACGUUUCCAACA | | [1039-1057] | [801-819] | [786-804] |
| 32 | GAAACCUAUUCUCGGAGGC | GCCUCCGAGAAUAGGUUUC | | [543-561] | [305-323] | [290-308] |
| 33 | AACCUAUUCUCGGAGGCUG | CAGCCUCCGAGAAUAGGUU | | [545-563] | [307-325] | [292-310] |
| 34 | GUGUUGGAAACGUCAGACC | GGUCUGACGUUUCCAACAC | | [1038-1056] | [800-818] | [785-803] |
| 35 | AGUUGGGACGAAUUCAGGC | GCCUGAAUUCGUCCCAACU | | [381-399] | [125-143] | [125-143] |
| 36 | AACGUUUGCCUUCUCUGUG | CACAGAGAAGGCAAACGUU | | [326-344] | [70-88] | [70-88] |
| 37 | ACGUUUGCCUUCUCUGUGC | GCACAGAGAAGGCAAACGU | | [327-345] | [71-89] | [71-89] |
| 38 | AAGACCCUCAAGGAGACCU | AGGUCUCCUUGAGGGUCUU | | [405-423] | [152-170] | [152-170] |
| 39 | UUGGGACGAAUUCAGGCAG | CUGCCUGAAUUCGUCCCAA | | [383-401] | [127-145] | [127-145] |
| 40 | GUUCUCCCAAAGCUUCUCG | CGAGAAGCUUUGGGAGAAC | | [462-480] | [209-227] | [209-227] |
| 41 | UUUGCCUUCUCUGUGCGCU | AGCGCACAGAGAAGGCAAA | | [330-348] | [74-92] | [74-92] |

TABLE A13
NOXO2 19-mer oligomer pairs (NADPH oxidase organizer 2, NCF1)

| | | | | Human- |
|---|---|---|---|---|
| | | | Other | 115298671 |
| No. | Sense siRNA | AntiSense siRNA | Sp | ORF: 71-1243 |
| 1 | CCUGUACAUACGUGUUCUA | UAGAACACGUAUGUACAGG | | [1292-1310] |
| 2 | GGGCGAUCAAUCCAGAGAA | UUCUCUGGAUUGAUCGCCC | | [258-276 |
| 3 | GUACAUACGUGUUCUAUAG | CUAUAGAACACGUAUGUAC | | [1295-1313] |
| 4 | CCAUUGCCAACUACGAGAA | UUCUCGUAGUUGGCAAUGG | | [558-576 |
| 5 | ACAUACGUGUUCUAUAGAG | CUCUAUAGAACACGUAUGU | | [1297-1315] |
| 6 | CAAAAAAGCCAGAGACAUA | UAUGUCUCUGGCUUUUUUG | | [468-486 |
| 7 | ACAAAAAAGCCAGAGACAU | AUGUCUCUGGCUUUUUUGU | | [467-485 |
| 8 | GCCUGUACAUACGUGUUCU | AGAACACGUAUGUACAGGC | | [1291-1309] |
| 9 | CCGUCCAUGUACCUGCAAA | UUUGCAGGUACAUGGACGG | | [896-914 |
| 10 | CUGUACAUACGUGUUCUAU | AUAGAACACGUAUGUACAG | | [1293-1311] |
| 11 | UCAGAUGAAAGCAAAGCGA | UCGCUUUGCUUUCAUCUGA | | [658-676 |
| 12 | CGAUCAAUCCAGAGAACAG | CUGUUCUCUGGAUUGAUCG | | [261-279 |
| 13 | CGUCCAUGUACCUGCAAAA | UUUUGCAGGUACAUGGACG | | [897-915 |
| 14 | GUGGUUCUGUCAGAUGAAA | UUUCAUCUGACAGAACCAC | | [649-667 |
| 15 | GCCAACUACGAGAAGACCU | AGGUCUUCUCGUAGUUGGC | | [563-581 |
| 16 | ACAUACUUGAUGCCCAAAG | CUUUGGGCAUCAAGUAUGU | | [482-500 |
| 17 | CCAGAGACAUACUUGAUGC | GCAUCAAGUAUGUCUCUGG | | [476-494 |
| 18 | GGACAACCAGACAAAAAAG | CUUUUUUGUCUGGUUGUCC | | [457-475 |
| 19 | GCGAUCAAUCCAGAGAACA | UGUUCUCUGGAUUGAUCGC | | [260-278 |
| 20 | UGCCUGUACAUACGUGUUC | GAACACGUAUGUACAGGCA | | [1290-1308] |
| 21 | GCCAUUGCCAACUACGAGA | UCUCGUAGUUGGCAAUGGC | | [557-575 |
| 22 | AGCCAGAGACAUACUUGAU | AUCAAGUAUGUCUCUGGCU | | [474-492 |
| 23 | AAAAGCCAGAGACAUACUU | AAGUAUGUCUCUGGCUUUU | | [471-489 |
| 24 | GGUGGUUCUGUCAGAUGAA | UUCAUCUGACAGAACCACC | | [648-666 |
| 25 | UGUACAUGUUCCUGGUGAA | UUCACCAGGAACAUGUACA | GP | [144-162 |
| 26 | GUGCCUGUACAUACGUGUU | AACACGUAUGUACAGGCAC | | [1289-1307] |
| 27 | GCUACUUCCCGUCCAUGUA | UACAUGGACGGGAAGUAGC | | [888-906 |
| 28 | GUGUACAUGUUCCUGGUGA | UCACCAGGAACAUGUACAC | GP | [143-161 |
| 29 | CGUGCCUGUACAUACGUGU | ACACGUAUGUACAGGCACG | | [1288-1306] |
| 30 | UGGUGGGUCAUCAGGAAAG | CUUUCCUGAUGACCCACCA | | [857-875 |
| 31 | ACACCUUCAUCCGUCACAU | AUGUGACGGAUGAAGGUGU | | [78-96 |
| 32 | UGGUGGUUCUGUCAGAUGA | UCAUCUGACAGAACCACCA | | [647-665 |
| 33 | UGGUGGAGGUCGUAGAGAA | UUCUCUACGACCUCCACCA | | [615-633 |
| 34 | GUCGUAGAGAAGAGCGAGA | UCUCGCUCUUCUCUACGAC | | [623-641 |
| 35 | AGAGUACCGCGACAGACAU | AUGUCUGUCGCGGUACUCU | | [507-525 |
| 36 | ACGAGUUCCAUAAAACCUU | AAGGUUUUAUGGAACUCGU | | [213-231 |
| 37 | GGUUGGUGGUUCUGUCAGA | UCUGACAGAACCACCAACC | | [644-662 |
| 38 | GCAACAGCGUCCGUUUUCU | AGAAAACGGACGCUGUUGC | | [1047-1065 |
| 39 | GUUGGUGGUUCUGUCAGAU | AUCUGACAGAACCACCAAC | | [645-663 |
| 40 | UGGAGGUCGUAGAGAAGAG | CUCUUCUCUACGACCUCCA | | [618-636 |
| 41 | CCAGACAAAAAAGCCAGAG | CUCUGGCUUUUUUGUCUGG | | [463-481 |
| 42 | CCACGGACAACCAGACAAA | UUUGUCUGGUUGUCCGUGG | | [453-471 |
| 43 | AUCUACGAGUUCCAUAAAA | UUUUAUGGAACUCGUAGAU | Rat, Ms | [209-227 |
| 44 | UGUCGGAGAAGGUGGUCUA | UAGACCACCUUCUCCGACA | Rat, Ms | [174-192 |
| 45 | CUUCCUCUUUCCAGUGCAU | AUGCACUGGAAAGAGGAAG | | [4-22] |
| 46 | AGGUCAUUCACAAGCUCCU | AGGAGCUUGUGAAUGACCU | | [831-849 |
| 47 | CAGACAAAAAAGCCAGAGA | UCUCUGGCUUUUUUGUCUG | | [464-482 |
| 48 | CAGCUCCCAAGUGGUUUGA | UCAAACCACUUGGGAGCUG | | [297-315 |
| 49 | GCCAGCACUAUGUGUACAU | AUGUACACAUAGUGCUGGC | Ms | [132-150 |
| 50 | CGGACAACCAGACAAAAAA | UUUUUUGUCUGGUUGUCCG | | [456-474 |
| 51 | GAGUUCCAUAAAACCUUAA | UUAAGGUUUUAUGGAACUC | | [215-233 |
| 52 | AGACAAAAAAGCCAGAGAC | GUCUCUGGCUUUUUUGUCU | | [465-483 |
| 53 | ACCAGACAAAAAAGCCAGA | UCUGGCUUUUUUGUCUGGU | | [462-480 |
| 54 | ACGGACAACCAGACAAAAA | UUUUUGUCUGGUUGUCCGU | | [455-473 |
| 55 | UCAGGAAAGACGACGUCAC | GUGACGUCGUCUUUCCUGA | | [867-885 |
| 56 | CCAACUACGAGAAGACCUC | GAGGUCUUCUCGUAGUUGG | | [564-582 |
| 57 | AGACAUACUUGAUGCCCAA | UUGGGCAUCAAGUAUGUCU | | [480-498 |
| 58 | GGCACACUUACCGAGUACU | AGUACUCGGUAAGUGUGCC | | [344-362 |
| 59 | CCUCUUUCCAGUGCAUUUA | UAAAUGCACUGGAAAGAGG | | [7-25] |
| 60 | UUCUGUCAGAUGAAAGCAA | UUGCUUUCAUCUGACAGAA | | [653-671 |
| 61 | CACGGACAACCAGACAAAA | UUUUGUCUGGUUGUCCGUG | | [454-472 |
| 62 | CGACUUCCUCUUUCCAGUG | CACUGGAAAGAGGAAGUCG | | [1-19] |
| 63 | UCAAUCCAGAGAACAGGAU | AUCCUGUUCUCUGGAUUGA | | [264-282 |
| 64 | ACCUCCUCGACUUCUUCAA | UUGAAGAAGUCGAGGAGGU | | [408-426 |
| 65 | CUCUUUCCAGUGCAUUUAA | UUAAAUGCACUGGAAAGAG | | [8-26] |
| 66 | UCUGUCAGAUGAAAGCAAA | UUUGCUUUCAUCUGACAGA | | [654-672 |
| 67 | CCCAAAGAUGGCAAGAGUA | UACUCUUGCCAUCUUUGGG | | [494-512 |
| 68 | UGAAAUGGCAGGACCUGUC | GACAGGUCCUGCCAUUUCA | | [159-177 |
| 69 | AGCACUAUGUGUACAUGUU | AACAUGUACACAUAGUGCU | Ms | [135-153 |
| 70 | CCAGCACUAUGUGUACAUG | CAUGUACACAUAGUGCUGG | Ms | [133-151 |
| 71 | UGGGCUUUGAGAAGCGCUU | AAGCGCUUCUCAAAGCCCA | | [105-123 |
| 72 | UGAUGCCCAAAGAUGGCAA | UUGCCAUCUUUGGGCAUCA | | [489-507 |
| 73 | CAACCAGACAAAAAAGCCA | UGGCUUUUUUGUCUGGUUG | | [460-478 |
| 74 | CCAGAGAACAGGAUCAUCC | GGAUGAUCCUGUUCUCUGG | | [269-287 |
| 75 | GCAAAAGUCAGGGCAAGAC | GUCUUGCCCUGACUUUUGC | | [910-928 |
| 76 | GCUUUGAGAAGCGCUUCGU | ACGAAGCGCUUCUCAAAGC | | [108-126 |
| 77 | AGCCCAACUAUGCAGGUGA | UCACCUGCAUAGUUGGGCU | | [738-756 |
| 78 | GAGACAUACUUGAUGCCCA | UGGGCAUCAAGUAUGUCUC | | [479-497 |
| 79 | UCCUCGACUUCUUCAAGGU | ACCUUGAAGAAGUCGAGGA | | [411-429 |
| 80 | UCCUCUUUCCAGUGCAUUU | AAAUGCACUGGAAAGAGGA | | [6-24] |
| 81 | GCACUAUGUGUACAUGUUC | GAACAUGUACACAUAGUGC | Ms | [136-154 |
| 82 | UCAUCAGGAAAGACGACGU | ACGUCGUCUUUCCUGAUGA | | [864-882 |
| 83 | CAACUAUGCAGGUGAGCCA | UGGCUCACCUGCAUAGUUG | | [742-760 |
| 84 | GACAUACUUGAUGCCCAAA | UUUGGGCAUCAAGUAUGUC | | [481-499 |
| 85 | AUCCAGAGAACAGGAUCAU | AUGAUCCUGUUCUCUGGAU | | [267-285 |
| 86 | UGCCCAAAGAUGGCAAGAG | CUCUUGCCAUCUUUGGGCA | | [492-510 |
| 87 | CCAGUGCAUUUAAGGCGCA | UGCGCCUUAAAUGCACUGG | | [14-32] |
| 88 | CCUGCAAAAGUCAGGGCAA | UUGCCCUGACUUUUGCAGG | | [907-925 |
| 89 | GGGUCAUCAGGAAAGACGA | UCGUCUUUCCUGAUGACCC | | [861-879 |
| 90 | CUGCAAAAGUCAGGGCAAG | CUUGCCCUGACUUUUGCAG | | [908-926 |
| 91 | AGACCCUGAGCCCAACUAU | AUAGUUGGGCUCAGGGUCU | | [730-748 |
| 92 | AGCCAGCACUAUGUGUACA | UGUACACAUAGUGCUGGCU | Ms | [131-149 |
| 93 | CUAUGCAGGUGAGCCAUAC | GUAUGGCUCACCUGCAUAG | | [745-763 |
| 94 | UCAUUCACAAGCUCCUGGA | UCCAGGAGCUUGUGAAUGA | | [834-852 |
| 95 | UGAGGUCAUUCACAAGCUC | GAGCUUGUGAAUGACCUCA | | [829-847 |
| 96 | GACACCUUCAUCCGUCACA | UGUGACGGAUGAAGGUGUC | | [77-95 |

TABLE A14
NOXO2 19-mer oligomer pairs (NADPH oxidase organizer 2, NCF1)

| | | | | Human-115298671 |
|---|---|---|---|---|
| No. | Sense siRNA | AntiSense siRNA | Other Sp | ORF: 71-1243 |
| 1 | GAUCUACGAGUUCCAUAAA | UUUAUGGAACUCGUAGAUC | Rat, Ms | [208-226] |
| 2 | CCGAGAUCUACGAGUUCCA | UGGAACUCGUAGAUCUCGG | Rat, Ms | [204-222] |
| 3 | CCAUCAUCCUGCAGACGUA | UACGUCUGCAGGAUGAUGG | | [534-552] |
| 4 | AGAUCUACGAGUUCCAUAA | UUAUGGAACUCGUAGAUCU | Rat, Ms | [207-225] |
| 5 | CAGGAAAGACGACGUCACA | UGUGACGUCGUCUUUCCUG | | [868-886] |
| 6 | CAUAAAACCUUAAAAGAAA | UUUCUUUUAAGGUUUUAUG | | [221-239] |
| 7 | CGAGUUCCAUAAAACCUUA | UAAGGUUUUAUGGAACUCG | | [214-232] |
| 8 | CCAUAAAACCUUAAAAGAA | UUCUUUUAAGGUUUUAUGG | | [220-238] |
| 9 | CGAGAUCUACGAGUUCCAU | AUGGAACUCGUAGAUCUCG | Rat, Ms | [205-223] |
| 10 | GGUGGGUCAUCAGGAAAGA | UCUUUCCUGAUGACCCACC | | [858-876] |
| 11 | CAUACUUGAUGCCCAAAGA | UCUUUGGGCAUCAAGUAUG | | [483-501] |
| 12 | GUUCCAUAAAACCUUAAAA | UUUUAAGGUUUUAUGGAAC | | [217-235] |
| 13 | GCUUCACCGAGAUCUACGA | UCGUAGAUCUCGGUGAAGC | | [198-216] |
| 14 | GAGAUCUACGAGUUCCAUA | UAUGGAACUCGUAGAUCUC | Rat, Ms | [206-224] |
| 15 | CUGUUGAGGUCAUUCACAA | UUGUGAAUGACCUCAACAG | | [825-843] |
| 16 | AAGAAAUGUUCCCUAUUGA | UCAAUAGGGAACAUUUCUU | | [234-252] |
| 17 | CUUUGAGAAGCGCUUCGUA | UACGAAGCGCUUCUCAAAG | | [109-127] |
| 18 | AGGGCACACUUACCGAGUA | UACUCGGUAAGUGUGCCCU | | [342-360] |
| 19 | UGUACAUACGUGUUCUAUA | UAUAGAACACGUAUGUACA | | [1294-1312] |
| 20 | CUGGUGGGUCAUCAGGAAA | UUUCCUGAUGACCCACCAG | | [856-874] |
| 21 | CAUGUACCUGCAAAAGUCA | UGACUUUUGCAGGUACAUG | | [901-919] |
| 22 | AGUUCCAUAAAACCUUAAA | UUUAAGGUUUUAUGGAACU | | [216-234] |
| 23 | CCAUGUACCUGCAAAAGUC | GACUUUUGCAGGUACAUGG | | [900-918] |
| 24 | GGCGAUCAAUCCAGAGAAC | GUUCUCUGGAUUGAUCGCC | | [259-277] |
| 25 | CUGUCAGAUGAAAGCAAAG | CUUUGCUUUCAUCUGACAG | | [655-673] |
| 26 | GUACAUGUUCCUGGUGAAA | UUUCACCAGGAACAUGUAC | | [145-163] |
| 27 | AAAGCAAAGCGAGGCUGGA | UCCAGCCUCGCUUUGCUUU | | [665-683] |
| 28 | AAGAGUACCGCGACAGACA | UGUCUGUCGCGGUACUCUU | | [506-524] |
| 29 | AGGUCGUAGAGAAGAGCGA | UCGCUCUUCUCUACGACCU | | [621-639] |
| 30 | AAGACGACGUCACAGGCUA | UAGCCUGUGACGUCGUCUU | | [873-891] |
| 31 | GGUCAUCAGGAAAGACGAC | GUCGUCUUUCCUGAUGACC | | [862-880] |
| 32 | UCCAUAAAACCUUAAAAGA | UCUUUUAAGGUUUUAUGGA | | [219-237] |
| 33 | GCUGUUGAGGUCAUUCACA | UGUGAAUGACCUCAACAGC | | [824-842] |
| 34 | ACAUGUUCCUGGUGAAAUG | CAUUUCACCAGGAACAUGU | | [147-165] |
| 35 | GUGGAGGUCGUAGAGAAGA | UCUUCUCUACGACCUCCAC | | [617-635] |
| 36 | UGCAAAAGUCAGGGCAAGA | UCUUGCCCUGACUUUUGCA | | [909-927] |
| 37 | UCACCGAGAUCUACGAGUU | AACUCGUAGAUCUCGGUGA | Rat, Ms | [201-219] |
| 38 | UAAAAGAAAUGUUCCCUAU | AUAGGGAACAUUUCUUUUA | | [231-249] |
| 39 | CAGAGAACAGGAUCAUCCC | GGGAUGAUCCUGUUCUCUG | | [270-288] |
| 40 | AGAAAUGUUCCCUAUUGAG | CUCAAUAGGGAACAUUUCU | | [235-253] |
| 41 | AAACCUUAAAAGAAAUGUU | AACAUUUCUUUUAAGGUUU | | [225-243] |
| 42 | AGAUGAAAGCAAAGCGAGG | CCUCGCUUUGCUUUCAUCU | | [660-678] |
| 43 | CACCGAGAUCUACGAGUUC | GAACUCGUAGAUCUCGGUG | Rat, Ms | [202-220] |
| 44 | CAGAUGAAAGCAAAGCGAG | CUCGCUUUGCUUUCAUCUG | | [659-677] |
| 45 | CAUCAAGGCCUACACUGCU | AGCAGUGUAGGCCUUGAUG | | [769-787] |
| 46 | CUACGAGUUCCAUAAAACC | GGUUUUAUGGAACUCGUAG | | [211-229] |
| 47 | GGUUCUGUCAGAUGAAAGC | GCUUUCAUCUGACAGAACC | | [651-669] |
| 48 | AAGCAAAGCGAGGCUGGAU | AUCCAGCCUCGCUUUGCUU | | [666-684] |
| 49 | AAUCCAGAGAACAGGAUCA | UGAUCCUGUUCUCUGGAUU | | [266-284] |
| 50 | AAAAGAAAUGUUCCCUAUU | AAUAGGGAACAUUUCUUUU | | [232-250] |
| 51 | CACCUCCUCGACUUCUUCA | UGAAGAAGUCGAGGAGGUG | | [407-425] |
| 52 | AAGCUGUUGAGGUCAUUCA | UGAAUGACCUCAACAGCUU | | [822-840] |
| 53 | AUACUUGAUGCCCAAAGAU | AUCUUUGGGCAUCAAGUAU | | [484-502] |
| 54 | GUUCUGUCAGAUGAAAGCA | UGCUUUCAUCUGACAGAAC | | [652-670] |
| 55 | UUUAAGGCGCAGCCUGGAA | UUCCAGGCUGCGCCUUAAA | | [22-40] |
| 56 | AAUGUUCCCUAUUGAGGCA | UGCCUCAAUAGGGAACAUU | | [238-256] |
| 57 | AGCUGUUGAGGUCAUUCAC | GUGAAUGACCUCAACAGCU | | [823-841] |
| 58 | CCUCCUCGACUUCUUCAAG | CUUGAAGAAGUCGAGGAGG | | [409-427] |
| 59 | ACAGCGUCCGUUUUCUGCA | UGCAGAAAACGGACGCUGU | | [1050-1068] |
| 60 | AUCAAUCCAGAGAACAGGA | UCCUGUUCUCUGGAUUGAU | | [263-281] |
| 61 | UACAUACGUGUUCUAUAGA | UCUAUAGAACACGUAUGUA | | [1296-1314] |
| 62 | GUGAAGCUGUUGAGGUCAU | AUGACCUCAACAGCUUCAC | | [819-837] |
| 63 | UCUACGAGUUCCAUAAAAC | GUUUUAUGGAACUCGUAGA | | [210-228] |
| 64 | GCACACUUACCGAGUACUG | CAGUACUCGGUAAGUGUGC | | [345-363] |
| 65 | CGUGUUCUAUAGAGCCUGG | CCAGGCUCUAUAGAACACG | | [1302-1320] |
| 66 | AUUUAAGGCGCAGCCUGGA | UCCAGGCUGCGCCUUAAAU | | [21-39] |
| 67 | UGGUUCUGUCAGAUGAAAG | CUUUCAUCUGACAGAACCA | | [650-668] |
| 68 | UCGACUUCUUCAAGGUGCG | CGCACCUUGAAGAAGUCGA | | [414-432] |
| 69 | UUAAAAGAAAUGUUCCCUA | UAGGGAACAUUUCUUUUAA | | [230-248] |
| 70 | GGUGAAGCUGUUGAGGUCA | UGACCUCAACAGCUUCACC | | [818-836] |
| 71 | AAAAGUCAGGGCAAGACGU | ACGUCUUGCCCUGACUUUU | | [912-930] |
| 72 | UCGAGGGUGAAGCUGUUGA | UCAACAGCUUCACCCUCGA | | [813-831] |
| 73 | CCUCGACUUCUUCAAGGUG | CACCUUGAAGAAGUCGAGG | | [412-430] |
| 74 | UGAAGCUGUUGAGGUCAUU | AAUGACCUCAACAGCUUCA | | [820-838] |
| 75 | ACGACGUCACAGGCUACUU | AAGUAGCCUGUGACGUCGU | | [876-894] |
| 76 | AAGACCCUGAGCCCAACUA | UAGUUGGGCUCAGGGUCUU | | [729-747] |
| 77 | UGUUGAGGUCAUUCACAAG | CUUGUGAAUGACCUCAACA | | [826-844] |
| 78 | AAAGAAAUGUUCCCUAUUG | CAAUAGGGAACAUUUCUUU | | [233-251] |
| 79 | AAGUCAGGGCAAGACGUGU | ACACGUCUUGCCCUGACUU | | [914-932] |
| 80 | GUGGGUCAUCAGGAAAGAC | GUCUUUCCUGAUGACCCAC | | [859-877] |
| 81 | CACACUUACCGAGUACUGC | GCAGUACUCGGUAAGUGUG | | [346-364] |
| 82 | CAAAAGUCAGGGCAAGACG | CGUCUUGCCCUGACUUUUG | | [911-929] |
| 83 | AGGAAAGACGACGUCACAG | CUGUGACGUCGUCUUUCCU | | [869-887] |
| 84 | ACUUCCUCUUUCCAGUGCA | UGCACUGGAAAGAGGAAGU | | [3-21] |
| 85 | AGGGUGAAGCUGUUGAGGU | ACCUCAACAGCUUCACCCU | | [816-834] |
| 86 | GUCCAUGUACCUGCAAAAG | CUUUUGCAGGUACAUGGAC | | [898-916] |
| 87 | GACAACCAGACAAAAAAGC | GCUUUUUUGUCUGGUUGUC | | [458-476] |
| 88 | CAACAGCGUCCGUUUUCUG | CAGAAAACGGACGCUGUUG | | [1048-1066] |
| 89 | GAUCAAUCCAGAGAACAGG | CCUGUUCUCUGGAUUGAUC | | [262-280] |
| 90 | UCCAUGUACCUGCAAAAGU | ACUUUUGCAGGUACAUGGA | | [899-917] |
| 91 | ACUUGAUGCCCAAAGAUGG | CCAUCUUUGGGCAUCAAGU | | [486-504] |
| 92 | CUACUUCCCGUCCAUGUAC | GUACAUGGACGGGAAGUAG | | [889-907] |
| 93 | GUCAGAUGAAAGCAAAGCG | CGCUUUGCUUUCAUCUGAC | | [657-675] |
| 94 | UGAGCCCAACUAUGCAGGU | ACCUGCAUAGUUGGGCUCA | | [736-754] |
| 95 | CAUCAGGAAAGACGACGUC | GACGUCGUCUUUCCUGAUG | | [865-883] |
| 96 | UACGAGUUCCAUAAAACCU | AGGUUUUAUGGAACUCGUA | | [212-230] |
| 97 | CCUUAAAAGAAAUGUUCCC | GGGAACAUUUCUUUUAAGG | | [228-246] |
| 98 | AUCAGGAAAGACGACGUCA | UGACGUCGUCUUUCCUGAU | | [866-884] |
| 99 | CAGUGCAUUUAAGGCGCAG | CUGCGCCUUAAAUGCACUG | | [15-33] |
| 100 | UUCCAUAAAACCUUAAAAG | CUUUUAAGGUUUUAUGGAA | | [218-236] |
| 101 | UCUUUCCAGUGCAUUUAAG | CUUAAAUGCACUGGAAAGA | | [9-27] |
| 102 | CUUCACCGAGAUCUACGAG | CUCGUAGAUCUCGGUGAAG | | [199-217] |
| 103 | CAUCAUCCUGCAGACGUAC | GUACGUCUGCAGGAUGAUG | | [535-553] |
| 104 | ACAACCAGACAAAAAAGCC | GGCUUUUUUGUCUGGUUGU | | [459-477] |
| 105 | ACCUGCAAAAGUCAGGGCA | UGCCCUGACUUUUGCAGGU | | [906-924] |
| 106 | CACCUUCAUCCGUCACAUC | GAUGUGACGGAUGAAGGUG | | [79-97] |
| 107 | CAUACGUGUUCUAUAGAGC | GCUCUAUAGAACACGUAUG | | [1298-1316] |
| 108 | AAAGACGACGUCACAGGCU | AGCCUGUGACGUCGUCUUU | | [872-890] |
| 109 | ACUUCCCGUCCAUGUACCU | AGGUACAUGGACGGGAAGU | | [891-909] |
| 110 | AUGAAAGCAAAGCGAGGCU | AGCCUCGCUUUGCUUUCAU | | [662-680] |
| 111 | CAUUCACAAGCUCCUGGAC | GUCCAGGAGCUUGUGAAUG | | [835-853] |
| 112 | GAAAUGUUCCCUAUUGAGG | CCUCAAUAGGGAACAUUUC | | [236-254] |
| 113 | ACACUUACCGAGUACUGCG | CGCAGUACUCGGUAAGUGU | | [347-365] |
| 114 | CAGAGACAUACUUGAUGCC | GGCAUCAAGUAUGUCUCUG | | [477-495] |
| 115 | CAAUCCAGAGAACAGGAUC | GAUCCUGUUCUCUGGAUUG | | [265-283] |
| 116 | AGUACCGCGACAGACAUCA | UGAUGUCUGUCGCGGUACU | | [509-527] |
| 117 | CAUGUUCCUGGUGAAAUGG | CCAUUUCACCAGGAACAUG | | [148-166] |
| 118 | UACGUGUUCUAUAGAGCCU | AGGCUCUAUAGAACACGUA | | [1300-1318] |
| 119 | UUGAGGUCAUUCACAAGCU | AGCUUGUGAAUGACCUCAA | | [828-846] |
| 120 | CUCCUCGACUUCUUCAAGG | CCUUGAAGAAGUCGAGGAG | | [410-428] |
| 121 | AAAUGUUCCCUAUUGAGGC | GCCUCAAUAGGGAACAUUU | | [237-255] |
| 122 | CUUUCCAGUGCAUUUAAGG | CCUUAAAUGCACUGGAAAG | | [10-28] |
| 123 | UACAUGUUCCUGGUGAAAU | AUUUCACCAGGAACAUGUA | | [146-164] |
| 124 | CUCGACUUCUUCAAGGUGC | GCACCUUGAAGAAGUCGAG | | [413-431] |
| 125 | UUUGAGAAGCGCUUCGUAC | GUACGAAGCGCUUCUCAAA | | [110-128] |
| 126 | AACCUUAAAAGAAAUGUUC | GAACAUUUCUUUUAAGGUU | | [226-244] |
| 127 | AAAGUCAGGGCAAGACGUG | CACGUCUUGCCCUGACUUU | | [913-931] |
| 128 | GAUGAAAGCAAAGCGAGGC | GCCUCGCUUUGCUUUCAUC | | [661-679] |
| 129 | GAAGCUGUUGAGGUCAUUC | GAAUGACCUCAACAGCUUC | | [821-839] |
| 130 | CAACUACGAGAAGACCUCG | CGAGGUCUUCUCGUAGUUG | | [565-583] |
| 131 | UGCAUUUAAGGCGCAGCCU | AGGCUGCGCCUUAAAUGCA | | [18-36] |
| 132 | AUGUACCUGCAAAAGUCAG | CUGACUUUUGCAGGUACAU | | [902-920] |
| 133 | ACCGAGAUCUACGAGUUCC | GGAACUCGUAGAUCUCGGU | Rat, Ms | [203-221] |
| 134 | GGUCAUUCACAAGCUCCUG | CAGGAGCUUGUGAAUGACC | | [832-850] |
| 135 | CUUAAAAGAAAUGUUCCCU | AGGGAACAUUUCUUUUAAG | | [229-247] |
| 136 | UUCACCGAGAUCUACGAGU | ACUCGUAGAUCUCGGUGAA | Rat, Ms | [200-218] |
| 137 | ACCUUAAAAGAAAUGUUCC | GGAACAUUUCUUUUAAGGU | | [227-245] |
| 138 | UCCAGAGAACAGGAUCAUC | GAUGAUCCUGUUCUCUGGA | | [268-286] |
| 139 | UACUUGAUGCCCAAAGAUG | CAUCUUUGGGCAUCAAGUA | | [485-503] |
| 140 | UCGUAGAGAAGAGCGAGAG | CUCUCGCUCUUCUCUACGA | | [624-642] |
| 141 | UGGGUCAUCAGGAAAGACG | CGUCUUUCCUGAUGACCCA | | [860-878] |
| 142 | AAAACCUUAAAAGAAAUGU | ACAUUUCUUUUAAGGUUUU | | [224-242] |
| 143 | UCAAGGCCUACACUGCUGU | ACAGCAGUGUAGGCCUUGA | | [771-789] |
| 144 | GUACCUGCAAAAGUCAGGG | CCCUGACUUUUGCAGGUAC | | [904-922] |
| 145 | UUGAGAAGCGCUUCGUACC | GGUACGAAGCGCUUCUCAA | | [111-129] |
| 146 | ACGUGUUCUAUAGAGCCUG | CAGGCUCUAUAGAACACGU | | [1301-1319] |
| 147 | AGCUCCCAAGUGGUUUGAC | GUCAAACCACUUGGGAGCU | | [298-316] |
| 148 | UGUACCUGCAAAAGUCAGG | CCUGACUUUUGCAGGUACA | | [903-921] |
| 149 | AUACGUGUUCUAUAGAGCC | GGCUCUAUAGAACACGUAU | | [1299-1317] |
| 150 | UGUCAGAUGAAAGCAAAGC | GCUUUGCUUUCAUCUGACA | | [656-674] |
| 151 | AUCAAGGCCUACACUGCUG | CAGCAGUGUAGGCCUUGAU | | [770-788] |
| 152 | UGUUCCCUAUUGAGGCAGG | CCUGCCUCAAUAGGGAACA | | [240-258] |
| 153 | UACUUCCCGUCCAUGUACC | GGUACAUGGACGGGAAGUA | | [890-908] |
| 154 | UAAAACCUUAAAAGAAAUG | CAUUUCUUUUAAGGUUUUA | | [223-241] |
| 155 | GUCAUCAGGAAAGACGACG | CGUCGUCUUUCCUGAUGAC | | [863-881] |
| 156 | UGAAAGCAAAGCGAGGCUG | CAGCCUCGCUUUGCUUUCA | | [663-681] |
| 157 | AGUGCAUUUAAGGCGCAGC | GCUGCGCCUUAAAUGCACU | | [16-34] |
| 158 | AACAGCGUCCGUUUUCUGC | GCAGAAAACGGACGCUGUU | | [1049-1067] |
| 159 | UUGGUGGUUCUGUCAGAUG | CAUCUGACAGAACCACCAA | | [646-664] |
| 160 | AACUACGAGAAGACCUCGG | CCGAGGUCUUCUCGUAGUU | | [566-584] |
| 161 | ACCUUCAUCCGUCACAUCG | CGAUGUGACGGAUGAAGGU | | [80-98] |
| 162 | GUUGAGGUCAUUCACAAGC | GCUUGUGAAUGACCUCAAC | | [827-845] |
| 163 | UUCCUCUUUCCAGUGCAUU | AAUGCACUGGAAAGAGGAA | | [5-23] |
| 164 | UUUCCAGUGCAUUUAAGGC | GCCUUAAAUGCACUGGAAA | | [11-29] |
| 165 | UCCAGUGCAUUUAAGGCGC | GCGCCUUAAAUGCACUGGA | | [13-31] |
| 166 | GAGGUCAUUCACAAGCUCC | GGAGCUUGUGAAUGACCUC | | [830-848] |
| 167 | GACUUCCUCUUUCCAGUGC | GCACUGGAAAGAGGAAGUC | | [2-20] |
| 168 | UACCUGCAAAAGUCAGGGC | GCCCUGACUUUUGCAGGUA | | [905-923] |
| 169 | AGAGACAUACUUGAUGCCC | GGGCAUCAAGUAUGUCUCU | | [478-496] |
| 170 | GUCAUUCACAAGCUCCUGG | CCAGGAGCUUGUGAAUGAC | | [833-851] |
| 171 | AUCAUCCUGCAGACGUACC | GGUACGUCUGCAGGAUGAU | | [536-554] |
| 172 | UUCCAGUGCAUUUAAGGCG | CGCCUUAAAUGCACUGGAA | | [12-30] |
| 173 | AUUCACAAGCUCCUGGACG | CGUCCAGGAGCUUGUGAAU | | [836-854] |
| 174 | AUGUUCCCUAUUGAGGCAG | CUGCCUCAAUAGGGAACAU | | [239-257] |

TABLE A15
NOXA1 19-mer oligomer pairs

| | | | | Human- |
|---|---|---|---|---|
| | | | | 41393186 |
| No. | Sense siRNA | AntiSense siRNA | Other Sp | ORF: 136-1587 |
| 1 | GCCCUAAUGAUGCUGUGUC | GACACAGCAUCAUUAGGGC | | [1581-1599 |
| 2 | GGAGAUCAGCCCUAAUGAU | AUCAUUAGGGCUGAUCUCC | | [1573-1591 |
| 3 | GGGAGGUGCUACACAAUGU | ACAUUGUGUAGCACCUCCC | Chmp | [500-518 |
| 4 | AGGAGGCUCUGUCUGACUU | AAGUCAGACAGAGCCUCCU | | [401-419 |
| 5 | CUAAUGAUGCUGUGUCCAU | AUGGACACAGCAUCAUUAG | | [1584-1602 |
| 6 | CCCUAAUGAUGCUGUGUCC | GGACACAGCAUCAUUAGGG | | [1582-1600 |
| 7 | GGAGCAAGUUGGCAAACAG | CUGUUUGCCAACUUGCUCC | Chmp | [951-969 |
| 8 | GGAGGUGCUACACAAUGUG | CACAUUGUGUAGCACCUCC | Chmp | [501-519 |
| 9 | AUGAUGCUGUGUCCAUGAU | AUCAUGGACACAGCAUCAU | | [1587-1605] |
| 10 | GAGGCUCUGUCUGACUUCU | AGAAGUCAGACAGAGCCUC | | [403-421 |
| 11 | CUGGGAGGUGCUACACAAU | AUUGUGUAGCACCUCCCAG | | [498-516 |
| 12 | UGGAGCAAGUUGGCAAACA | UGUUUGCCAACUUGCUCCA | Chmp | [950-968 |
| 13 | GGUGGAGCAAGUUGGCAAA | UUUGCCAACUUGCUCCACC | Chmp | [948-966 |
| 14 | GUGUCCAUGAUGCUUUUAA | UUAAAAGCAUCAUGGACAC | Chmp | [1595-1613] |
| 15 | UGUCCAUGAUGCUUUUAAU | AUUAAAAGCAUCAUGGACA | Chmp | [1596-1614] |
| 16 | GUCCAUGAUGCUUUUAAUA | UAUUAAAAGCAUCAUGGAC | Chmp | [1597-1615] |
| 17 | AGGUGGAGCAAGUUGGCAA | UUGCCAACUUGCUCCACCU | Chmp | [947-965 |
| 18 | CUGUGUCCAUGAUGCUUUU | AAAAGCAUCAUGGACACAG | Chmp | [1593-1611] |
| 19 | GCUGUGUCCAUGAUGCUUU | AAAGCAUCAUGGACACAGC | Chmp | [1592-1610] |
| 20 | UGACCAAGGACACCUGCAU | AUGCAGGUGUCCUUGGUCA | Rat | [329-347 |
| 21 | GCAUUUGACCAAGCCGUGA | UCACGGCUUGGUCAAAUGC | | [313-331 |
| 22 | CAGCCCUAAUGAUGCUGUG | CACAGCAUCAUUAGGGCUG | | [1579-1597 |
| 23 | UCAGCCCUAAUGAUGCUGU | ACAGCAUCAUUAGGGCUGA | | [1578-1596 |
| 24 | GGGAGAUCAGCCCUAAUGA | UCAUUAGGGCUGAUCUCCC | | [1572-1590 |
| 25 | ACUUCCAGCUGGCAAGGUU | AACCUUGCCAGCUGGAAGU | | [380-398 |
| 26 | AGGGAGAUCAGCCCUAAUG | CAUUAGGGCUGAUCUCCCU | | [1571-1589 |
| 27 | UGGGCAACUCAGUUACCUA | UAGGUAACUGAGUUGCCCA | Chmp | [1188-1206 |
| 28 | GGGCAACUCAGUUACCUAG | CUAGGUAACUGAGUUGCCC | Chmp | [1189-1207 |
| 29 | AGCUUGGGCAACUCAGUUA | UAACUGAGUUGCCCAAGCU | Chmp | [1184-1202 |
| 30 | CCAGGUCCCUAAUCAUGGA | UCCAUGAUUAGGGACCUGG | Chmp | [830-848 |
| 31 | CAGGGAGAUCAGCCCUAAU | AUUAGGGCUGAUCUCCCUG | | [1570-1588 |
| 32 | GCAACUCAGUUACCUAGCC | GGCUAGGUAACUGAGUUGC | Chmp | [1191-1209 |
| 33 | CAUGAUGCCAGGUCCCUAA | UUAGGGACCUGGCAUCAUG | Chmp | [823-841 |
| 34 | UGCUACACAAUGUGGCGUC | GACGCCACAUUGUGUAGCA | Chmp | [506-524 |
| 35 | GCUUGGGCAACUCAGUUAC | GUAACUGAGUUGCCCAAGC | Chmp | [1185-1203 |
| 36 | GCAAGUUGGCAAACAGGCU | AGCCUGUUUGCCAACUUGC | Chmp | [954-972 |
| 37 | GGUCCCUAAUCAUGGACUC | GAGUCCAUGAUUAGGGACC | Chmp | [833-851 |
| 38 | AGGUCCCUAAUCAUGGACU | AGUCCAUGAUUAGGGACCU | Chmp | [832-850 |

TABLE A16
NOXA1 - 19-mer oligomer pairs NADPH oxidase activator 1

| | | | Other | Human-41393186 |
|---|---|---|---|---|
| No. | Sense siRNA | AntiSense siRNA | Sp | ORF: 136-1587 |
| 1 | CCAUGAUGCUUUUAAUAAA | UUUAUUAAAAGCAUCAUGG | Chmp | [1599-1617](19/19) |
| 2 | CAUGAUGCUUUUAAUAAAA | UUUUAUUAAAAGCAUCAUG | Chmp | [1600-1618](19/19) |
| 3 | GAUGCUUUUAAUAAAAACA | UGUUUUUAUUAAAAGCAUC | Chmp | [1603-1621](19/19) |
| 4 | UGAUGCUUUUAAUAAAAAC | GUUUUUAUUAAAAGCAUCA | Chmp | [1602-1620](19/19) |
| 5 | UGCUUUUAAUAAAAACAAC | GUUGUUUUUAUUAAAAGCA | Chmp | [1605-1623](19/19) |
| 6 | UCCAUGAUGCUUUUAAUAA | UUAUUAAAAGCAUCAUGGA | Chmp | [1598-1616](19/19) |
| 7 | ACCAGGAGCGAACCAUGAU | AUCAUGGUUCGCUCCUGGU | Chmp | [810-828](19/19 |
| 8 | UUGACCAAGCCGUGACCAA | UUGGUCACGGCUUGGUCAA | | [317-335](19/19 |
| 9 | CAGCUUGGGCAACUCAGUU | AACUGAGUUGCCCAAGCUG | Chmp | [1183-1201](19/19 |
| 10 | GAUCAGCCCUAAUGAUGCU | AGCAUCAUUAGGGCUGAUC | | [1576-1594](19/19 |
| 11 | ACUUGGAGCCCGUGGAUUU | AAAUCCACGGGCUCCAAGU | | [713-731](19/19 |
| 12 | GAGAUCAGCCCUAAUGAUG | CAUCAUUAGGGCUGAUCUC | | [1574-1592](19/19 |
| 13 | GCUUUUAAUAAAAACAACC | GGUUGUUUUUAUUAAAAGC | Chmp | [1606-1624](19/19) |
| 14 | AGGCUCUGUCUGACUUCUG | CAGAAGUCAGACAGAGCCU | | [404-422](19/19 |
| 15 | AGAUCAGCCCUAAUGAUGC | GCAUCAUUAGGGCUGAUCU | | [1575-1593](19/19 |
| 16 | GGCAACUCAGUUACCUAGC | GCUAGGUAACUGAGUUGCC | Chmp | [1190-1208](19/19 |
| 17 | UUGGGCAACUCAGUUACCU | AGGUAACUGAGUUGCCCAA | Chmp | [1187-1205](19/19 |
| 18 | CUUGGGCAACUCAGUUACC | GGUAACUGAGUUGCCCAAG | Chmp | [1186-1204](19/19 |
| 19 | CAUUUGACCAAGCCGUGAC | GUCACGGCUUGGUCAAAUG | | [314-332](19/19 |
| 20 | CUUUUAAUAAAAACAACCC | GGGUUGUUUUUAUUAAAAG | Chmp | [1607-1625](19/19) |
| 21 | UCGAUGCAGAGACAGAGGU | ACCUCUGUCUCUGCAUCGA | | [881-899](19/19 |
| 22 | UUUGACCAAGCCGUGACCA | UGGUCACGGCUUGGUCAAA | | [316-334](19/19 |
| 23 | CAACUCAGUUACCUAGCCC | GGGCUAGGUAACUGAGUUG | Chmp | [1192-1210](19/19 |
| 24 | AUUUGACCAAGCCGUGACC | GGUCACGGCUUGGUCAAAU | | [315-333](19/19 |
| 25 | CAGGUCCCUAAUCAUGGAC | GUCCAUGAUUAGGGACCUG | Chmp | [831-849](19/19 |
| 26 | GUCCCUAAUCAUGGACUCC | GGAGUCCAUGAUUAGGGAC | Chmp | [834-852](19/19 |
| 27 | UCCCUAAUCAUGGACUCCC | GGGAGUCCAUGAUUAGGGA | Chmp | [835-853](19/19 |
| 28 | AUCAGCCCUAAUGAUGCUG | CAGCAUCAUUAGGGCUGAU | | [1577-1595](19/19 |

TABLE A17

NOXA2 19-mer oligomer pairs (NCF2, neutrophil
cytosolic factor 2, p67phox

| | | | | |
|---|---|---|---|---|
| | | | | Human-67189969 |
| No. | Sense siRNA | AntiSense siRNA | Other Sp | ORF: 253-1833 |

| | | | | |
|---|---|---|---|---|
| 1 | CCCUGCAACUACCUUGAAC | GUUCAAGGUAGUUGCAGGG | GP | [1120-1138 |
| 2 | GGAAAGUGAAAAAGCUGAU | AUCAGCUUUUUCACUUUCC | | [1572-1590 |
| 3 | GUGAAAAACUACUGCCUGA | UCAGGCAGUAGUUUUUCAC | Rat, Ms | [1501-1519 |
| 4 | GCAUUUGCAACACUGAGAA | UUCUCAGUGUUGCAAAUGC | | [79-97] |
| 5 | CCUUAUAGGUUACUCAUAA | UUAUGAGUAACCUAUAAGG | | [2334-2352] |
| 6 | CCAAGGAGUAAGUACAAAU | AUUUGUACUUACUCCUUGG | | [2265-2283] |
| 7 | UCAAAGGUGAAUGAAGAAU | AUUCUUCAUUCACCUUUGA | | [1714-1732 |
| 8 | ACCCAAGGAAAGUGAAAAA | UUUUUCACUUUCCUUGGGU | GP | [1566-1584 |
| 9 | GAAAAACUACUGCCUGACU | AGUCAGGCAGUAGUUUUUC | Ms | [1503-1521 |
| 10 | CCUUAAAGAAGCCUUGAUU | AAUCAAGGCUUCUUUAAGG | | [531-549 |
| 11 | CGCAACUACAGAUUUGGAA | UUCCAAAUCUGUAGUUGCG | | [1794-1812 |
| 12 | CCAAAGUUUUUGUUGAAGA | UCUUCAACAAAAACUUUGG | | [1772-1790 |
| 13 | GCUUUCCAGAUGAACCCAA | UUGGGUUCAUCUGGAAAGC | | [1553-1571 |
| 14 | AGGUGAAAAACUACUGCCU | AGGCAGUAGUUUUUCACCU | | [1499-1517 |
| 15 | UCCCUGCAACUACCUUGAA | UUCAAGGUAGUUGCAGGGA | GP | [1119-1137 |
| 16 | UCAUGUUCAACGGGCAGAA | UUCUGCCCGUUGAACAUGA | | [1091-1109 |
| 17 | ACCUUAAAGAAGCCUUGAU | AUCAAGGCUUCUUUAAGGU | | [530-548 |
| 18 | CCAGACAGAGAAAUAUGAU | AUCAUAUUUCUCUGUCUGG | | [498-516 |
| 19 | UGUACACUAUCCUGAAGAA | UUCUUCAGGAUAGUGUACA | | [389-407 |
| 20 | GGUGUUAUCAAAGGUGAAU | AUUCACCUUUGAUAACACC | | [1707-1725 |
| 21 | UGAUGCUAAUAACCAGACA | UGUCUGGUUAUUAGCAUCA | | [1587-1605 |
| 22 | CCCAAGGAAAGUGAAAAAG | CUUUUUCACUUUCCUUGGG | | [1567-1585 |
| 23 | GGCCAGGUGAAAAACUACU | AGUAGUUUUUCACCUGGCC | | [1495-1513 |
| 24 | ACACUGAGAAGUUAUCUUA | UAAGAUAACUUCUCAGUGU | | [88-106] |
| 25 | ACAUUGUCUUUGUCUUGAA | UUCAAGACAAAGACAAUGU | | [1046-1064 |
| 26 | UCGACCAAAUGAGAGACAA | UUGUCUCUCAUUUGGUCGA | | [801-819 |
| 27 | GGCUGCAUGUACACUAUCC | GGAUAGUGUACAUGCAGCC | | [382-400 |
| 28 | CGGACAAGAAGGACUGGAA | UUCCAGUCCUUCUUGUCCG | | [302-320 |
| 29 | GGUUACUCAUAAGGCAUAU | AUAUGCCUUAUGAGUAACC | | [2341-2359] |
| 30 | UCACACUGUAAACAUUCAU | AUGAAUGUUUACAGUGUGA | | [2157-2175] |
| 31 | CUAGACAAUGAUGUGAGUA | UACUCACAUCAUUGUCUAG | | [1975-1993] |
| 32 | AGAAAAUGAAGCCCUAUUA | UAAUAGGGCUUCAUUUUCU | | [1859-1877] |
| 33 | CCCAAAGUUUUUGUUGAAG | CUUCAACAAAAACUUUGGG | | [1771-1789 |
| 34 | GGAGGCACUCUUCAGUUAU | AUAACUGAAGAGUGCCUCC | | [1638-1656 |
| 35 | GAAGACAGCAUGAAGGAUG | CAUCCUUCAUGCUGUCUUC | | [1471-1489 |
| 36 | CACUCAAGGUGCACUACAA | UUGUAGUGCACCUUGAGUG | GP | [1310-1328 |
| 37 | AGACAAAAGAAGAGCUCCA | UGGAGCUCUUCUUUUGUCU | | [1013-1031 |
| 38 | CGACCAAAUGAGAGACAAG | CUUGUCUCUCAUUUGGUCG | | [802-820 |
| 39 | UGAUUCAGCUUCGAGGGAA | UUCCCUCGAAGCUGAAUCA | | [545-563 |
| 40 | CAGACAGAGAAAUAUGAUU | AAUCAUAUUUCUCUGUCUG | | [499-517 |
| 41 | GAAGCAUUAACCGAGACAA | UUGUCUCGGUUAAUGCUUC | | [437-455 |
| 42 | GCUGCAUGUACACUAUCCU | AGGAUAGUGUACAUGCAGC | | [383-401 |
| 43 | AGGUUACUCAUAAGGCAUA | UAUGCCUUAUGAGUAACCU | | [2340-2358] |
| 44 | UGAUGUGAGUACCCAGGAU | AUCCUGGGUACUCACAUCA | | [1983-2001] |
| 45 | GUAUACUGUACUGAGACAU | AUGUCUCAGUACAGUAUAC | | [1908-1926] |
| 46 | GAUGCUAAUAACCAGACAA | UUGUCUGGUUAUUAGCAUC | | [1588-1606 |
| 47 | GCCUGAGACAAAAGAAGAG | CUCUUCUUUUGUCUCAGGC | | [1008-1026 |
| 48 | AGAGAAAUAUGAUUUGGCU | AGCCAAAUCAUAUUUCUCU | | [504-522 |
| 49 | CCAGAAGCAUUAACCGAGA | UCUCGGUUAAUGCUUCUGG | | [434-452 |
| 50 | GGCCUCCUAGUUUCUACCU | AGGUAGAAACUAGGAGGCC | | [230-248] |
| 51 | CAUUCAUCAAGCUGUACAU | AUGUACAGCUUGAUGAAUG | | [2169-2187] |
| 52 | ACACUGUAAACAUUCAUCA | UGAUGAAUGUUUACAGUGU | | [2159-2177] |
| 53 | ACAAUGAUGUGAGUACCCA | UGGGUACUCACAUCAUUGU | | [1979-1997] |
| 54 | UAACCUACUAGACAAUGAU | AUCAUUGUCUAGUAGGUUA | | [1968-1986] |
| 55 | GGAUGUUUCACAAACUACA | UGUAGUUUGUGAAACAUCC | | [1833-1851] |
| 56 | UCGGAGAGAAGUCUAGGAU | AUCCUAGACUUCUCUCCGA | | [1818-1836 |
| 57 | AGGCACUCUUCAGUUAUGA | UCAUAACUGAAGAGUGCCU | | [1640-1658 |
| 58 | CAAGGAAAGUGAAAAAGCU | AGCUUUUUCACUUUCCUUG | | [1569-1587 |
| 59 | UGAACCCAAGGAAAGUGAA | UUCACUUUCCUUGGGUUCA | | [1563-1581 |
| 60 | ACAAAAAGAAGAGCCUAAG | CUUAGGCUCUUCUUUUUGU | | [1260-1278 |
| 61 | AACAAAAAGAAGAGCCUAA | UUAGGCUCUUCUUUUUGUU | | [1259-1277 |
| 62 | AGGCCAGAAACAAAAAGAA | UUCUUUUUGUUUCUGGCCU | | [1251-1269 |
| 63 | AGCUCCUCCUAGUUCCAAA | UUUGGAACUAGGAGGAGCU | | [1206-1224 |
| 64 | CAAAAGAAGAGCUCCAGGU | ACCUGGAGCUCUUCUUUUG | | [1016-1034 |
| 65 | CCGUGUGCUAUUUGGGUUU | AAACCCAAAUAGCACACGG | | [987-1005 |
| 66 | UGGUGGAUCAAGACAGUUU | AAACUGUCUUGAUCCACCA | | [875-893 |
| 67 | GGAAUGGAAAAAAGCUGAA | UUCAGCUUUUUUCCAUUCC | | [657-675 |
| 68 | CUCUCUGCUUCUUUCCUUU | AAAGGAAAGAAGCAGAGAG | | [4-22] |
| 69 | GACAGAGAAAUAUGAUUUG | CAAAUCAUAUUUCUCUGUC | | [501-519 |
| 70 | CUACCAGACAGAGAAAUAU | AUAUUUCUCUGUCUGGUAG | | [495-513 |
| 71 | GCAUUAACCGAGACAAGCA | UGCUUGUCUCGGUUAAUGC | | [440-458 |
| 72 | UGCAUGUACACUAUCCUGA | UCAGGAUAGUGUACAUGCA | | [385-403 |
| 73 | ACAAAAACAAGACACCCAA | UUGGGUGUCUUGUUUUUGU | | [2238-2256] |
| 74 | ACAUUCAUCAAGCUGUACA | UGUACAGCUUGAUGAAUGU | | [2168-2186] |
| 75 | CACUGUAAACAUUCAUCAA | UUGAUGAAUGUUUACAGUG | | [2160-2178] |
| 76 | CUACUAGACAAUGAUGUGA | UCACAUCAUUGUCUAGUAG | | [1972-1990] |
| 77 | ACAAAGCUGAAGAAAAUGA | UCAUUUUCUUCAGCUUUGU | | [1849-1867] |
| 78 | CAAAGUUUUUGUUGAAGAC | GUCUUCAACAAAAACUUUG | | [1773-1791 |
| 79 | GUGUUAUCAAAGGUGAAUG | CAUUCACCUUUGAUAACAC | | [1708-1726 |
| 80 | CUCUUCAGUUAUGAGGCUA | UAGCCUCAUAACUGAAGAG | | [1645-1663 |
| 81 | GGCUGGAACACACUAAGCU | AGCUUAGUGUGUUCCAGCC | | [1409-1427 |
| 82 | CACUGAGAAGUUAUCUUAA | UUAAGAUAACUUCUCAGUG | | [89-107] |
| 83 | GCCUAAGGAAGUGAAGCUC | GAGCUUCACUUCCUUAGGC | | [1272-1290 |
| 84 | GAAGAGCCUAAGGAAGUGA | UCACUUCCUUAGGCUCUUC | Rat | [1267-1285 |
| 85 | AGAAGAGCCUAAGGAAGUG | CACUUCCUUAGGCUCUUCU | Rat | [1266-1284 |
| 86 | CCAGGCCAGAAACAAAAAG | CUUUUUGUUUCUGGCCUGG | | [1249-1267 |
| 87 | GACAAAAGAAGAGCUCCAG | CUGGAGCUCUUCUUUUGUC | | [1014-1032 |
| 88 | GGCAUCUGUGGUGGAUCAA | UUGAUCCACCACAGAUGCC | GP | [867-885 |
| 89 | GACCAAAUGAGAGACAAGU | ACUUGUCUCUCAUUUGGUC | | [803-821 |
| 90 | GCUGUUUCGACCAAAUGAG | CUCAUUUGGUCGAAACAGC | | [795-813 |
| 91 | GACAUUCCAAAAUCGACAA | UUGUCGAUUUUGGAAUGUC | | [716-734 |
| 92 | UCUCUCUGCUUCUUUCCUU | AAGGAAAGAAGCAGAGAGA | | [3-21] |
| 93 | CUCAUAAGGCAUAUACAAA | UUUGUAUAUGCCUUAUGAG | | [2346-2364] |
| 94 | GUUACUCAUAAGGCAUAUA | UAUAUGCCUUAUGAGUAAC | | [2342-2360] |
| 95 | CACACUGUAAACAUUCAUC | GAUGAAUGUUUACAGUGUG | | [2158-2176] |
| 96 | ACCUACUAGACAAUGAUGU | ACAUCAUUGUCUAGUAGGU | | [1970-1988] |
| 97 | GUCUAGGAUGUUUCACAAA | UUUGUGAAACAUCCUAGAC | | [1828-1846 |
| 98 | AGUCUAGGAUGUUUCACAA | UUGUGAAACAUCCUAGACU | | [1827-1845 |
| 99 | CAACUACAGAUUUGGAAAG | CUUUCCAAAUCUGUAGUUG | | [1796-1814 |
| 100 | UGAAUGAAGAAUGGCUGGA | UCCAGCCAUUCUUCAUUCA | Rat, Ms | [1721-1739 |
| 101 | GGUGAAUGAAGAAUGGCUG | CAGCCAUUCUUCAUUCACC | | [1719-1737 |
| 102 | AGGUGAAUGAAGAAUGGCU | AGCCAUUCUUCAUUCACCU | | [1718-1736 |
| 103 | CCAGACAACAGAACCUCAG | CUGAGGUUCUGUUGUCUGG | | [1599-1617 |
| 104 | GAAAGUGAAAAAGCUGAUG | CAUCAGCUUUUUCACUUUC | | [1573-1591 |
| 105 | GGGCCAGGUGAAAAACUAC | GUAGUUUUUCACCUGGCCC | | [1494-1512 |
| 106 | GGCCAGAAACAAAAAGAAG | CUUCUUUUUGUUUCUGGCC | | [1252-1270 |
| 107 | GCAAUGAUAACUGGGCCAC | GUGGCCCAGUUAUCAUUGC | | [1070-1088 |
| 108 | ACAUUCCAAAAUCGACAAG | CUUGUCGAUUUUGGAAUGU | | [717-735 |
| 109 | AGCUGAUAGACUAUAAGAU | AUCUUAUAGUCUAUCAGCU | | [566-584 |
| 110 | CUAUCAAAGACCUUAAAGA | UCUUUAAGGUCUUUGAUAG | Ms | [521-539 |
| 111 | GAGAAAUAUGAUUUGGCUA | UAGCCAAAUCAUAUUUCUC | | [505-523 |
| 112 | ACCAGACAGAGAAAUAUGA | UCAUAUUUCUCUGUCUGGU | | [497-515 |
| 113 | AGAAGCAUUAACCGAGACA | UGUCUCGGUUAAUGCUUCU | | [436-454 |
| 114 | ACAUGACUGAAGCAGAGAA | UUCUCUGCUUCAGUCAUGU | | [407-425 |
| 115 | CCUUUUCUCUCUCAUGGUA | UACCAUGAGAGAGAAAAGG | | [18-36] |
| 116 | ACCUAAUCAUGUCCCUGGU | ACCAGGGACAUGAUUAGGU | | [245-263] |
| 117 | ACUGUAAACAUUCAUCAAG | CUUGAUGAAUGUUUACAGU | | [2161-2179] |
| 118 | GCCCUAUUACUUGUUUGUA | UACAAACAAGUAAUAGGGC | | [1869-1887] |
| 119 | GCGCAACUACAGAUUUGGA | UCCAAAUCUGUAGUUGCGC | | [1793-1811 |
| 120 | UGGUGUUAUCAAAGGUGAA | UUCACCUUUGAUAACACCA | | [1706-1724 |
| 121 | CUGGUGUUAUCAAAGGUGA | UCACCUUUGAUAACACCAG | | [1705-1723 |
| 122 | CCUGGUGUUAUCAAAGGUG | CACCUUUGAUAACACCAGG | | [1704-1722 |
| 123 | UAAUCCUGGUGUUAUCAAA | UUUGAUAACACCAGGAUUA | | [1700-1718 |
| 124 | AGGAAAGUGAAAAAGCUGA | UCAGCUUUUUCACUUUCCU | | [1571-1589 |
| 125 | CCAAGGAAAGUGAAAAAGC | GCUUUUUCACUUUCCUUGG | | [1568-1586 |
| 126 | GAACACACUAAGCUGAGCU | AGCUCAGCUUAGUGUGUUC | | [1414-1432 |
| 127 | GGAACACACUAAGCUGAGC | GCUCAGCUUAGUGUGUUCC | | [1413-1431 |
| 128 | ACUGAGAAGUUAUCUUAAG | CUUAAGAUAACUUCUCAGU | | [90-108] |
| 129 | AACACUGAGAAGUUAUCUU | AAGAUAACUUCUCAGUGUU | | [87-105] |
| 130 | UUGUCUUUGUCUUGAAGAA | UUCUUCAAGACAAAGACAA | | [1049-1067 |
| 131 | CUGAGACAAAAGAAGAGCU | AGCUCUUCUUUUGUCUCAG | | [1010-1028 |
| 132 | GGUGGAUCAAGACAGUUUC | GAAACUGUCUUGAUCCACC | | [876-894 |
| 133 | GCAUCUGUGGUGGAUCAAG | CUUGAUCCACCACAGAUGC | GP | [868-886 |
| 134 | CGAUGGAGUGUGUCUGGAA | UUCCAGACACACUCCAUCG | | [737-755 |
| 135 | GGGAACCAGCUGAUAGACU | AGUCUAUCAGCUGGUUCCC | Ms | [559-577 |
| 136 | ACAGAGAAAUAUGAUUUGG | CCAAAUCAUAUUUCUCUGU | | [502-520 |
| 137 | CUACUACCAGACAGAGAAA | UUUCUCUGUCUGGUAGUAG | | [492-510 |
| 138 | CUGAAGAACAUGACUGAAG | CUUCAGUCAUGUUCUUCAG | | [400-418 |
| 139 | UCCUGAAGAACAUGACUGA | UCAGUCAUGUUCUUCAGGA | | [398-416 |
| 140 | GGACAAGAAGGACUGGAAG | CUUCCAGUCCUUCUUGUCC | | [303-321 |
| 141 | UUACUCAUAAGGCAUAUAC | GUAUAUGCCUUAUGAGUAA | | [2343-2361] |
| 142 | GCUUGAAGUAACCUUAUAG | CUAUAAGGUUACUUCAAGC | | [2323-2341] |
| 143 | UACUGUAAUUGGCUCUUAA | UUAAGAGCCAAUUACAGUA | | [2303-2321] |
| 144 | UUACUGUAAUUGGCUCUUA | UAAGAGCCAAUUACAGUAA | | [2302-2320] |
| 145 | UCCUGUUUCUGAACCAUUA | UAAUGGUUCAGAAACAGGA | | [2286-2304] |
| 146 | GUAAGUACAAAUAUUCCUG | CAGGAAUAUUUGUACUUAC | | [2272-2290] |
| 147 | AAACCUAGCAUCUUACAAA | UUUGUAAGAUGCUAGGUUU | | [2224-2242] |
| 148 | UCAAGCUGUACAUUUGGUG | CACCAAAUGUACAGCUUGA | | [2175-2193] |
| 149 | CUGUAAACAUUCAUCAAGC | GCUUGAUGAAUGUUUACAG | | [2162-2180] |
| 150 | CCUGUUUCUUGCUCUGAGU | ACUCAGAGCAAGAAACAGG | | [2119-2137] |
| 151 | AGAGAGUCUUCCAGGUACU | AGUACCUGGAAGACUCUCU | | [2097-2115] |
| 152 | GAAAAGUGGAUGGAAGUGU | ACACUUCCAUCCACUUUUC | | [2064-2082] |
| 153 | UGAGACAUUACAGUUUGGA | UCCAAACUGUAAUGUCUCA | | [1919-1937] |
| 154 | CUGCUGUAUACUGUACUGA | UCAGUACAGUAUACAGCAG | | [1903-1921] |
| 155 | CCCUUCUGCUGUAUACUGU | ACAGUAUACAGCAGAAGGG | | [1898-1916] |
| 156 | AAGAAAAUGAAGCCCUAUU | AAUAGGGCUUCAUUUUCUU | | [1858-1876] |
| 157 | CGGAGAGAAGUCUAGGAUG | CAUCCUAGACUUCUCUCCG | | [1819-1837 |
| 158 | CUACAGAUUUGGAAAGCAC | GUGCUUUCCAAAUCUGUAG | | [1799-1817 |
| 159 | GUUGAAGACUGCGCAACUA | UAGUUGCGCAGUCUUCAAC | | [1783-1801 |
| 160 | GACAACAGAACCUCAGCUU | AAGCUGAGGUUCUGUUGUC | | [1602-1620 |
| 161 | ACACACUAAGCUGAGCUAU | AUAGCUCAGCUUAGUGUGU | | [1416-1434 |
| 162 | AACACACUAAGCUGAGCUA | UAGCUCAGCUUAGUGUGUU | | [1415-1433 |
| 163 | GAGAAGUUAUCUUAAGGGA | UCCCUUAAGAUAACUUCUC | | [93-111] |
| 164 | AGGAAGUGAAGCUCAGUGU | ACACUGAGCUUCACUUCCU | | [1277-1295 |
| 165 | CACCAGGCCAGAAACAAAA | UUUUGUUUCUGGCCUGGUG | | [1247-1265 |
| 166 | GGGAACAUUGUCUUUGUCU | AGACAAAGACAAUGUUCCC | | [1042-1060 |
| 167 | CCAGGGAACAUUGUCUUUG | CAAAGACAAUGUUCCCUGG | | [1039-1057 |
| 168 | GUGUGCUAUUUGGGUUUGU | ACAAACCCAAAUAGCACAC | | [989-1007 |
| 169 | ACCGUGUGCUAUUUGGGUU | AACCCAAAUAGCACACGGU | | [986-1004 |
| 170 | GUAGGGUUAUGAGUCAGUU | AACUGACUCAUAACCCUAC | | [34-52] |
| 171 | ACUUGGCAGUGGCUUACUU | AAGUAAGCCACUGCCAAGU | Rat | [458-476 |
| 172 | CAGAAGCAUUAACCGAGAC | GUCUCGGUUAAUGCUUCUG | | [435-453 |
| 173 | GAACAUGACUGAAGCAGAG | CUCUGCUUCAGUCAUGUUC | | [405-423 |
| 174 | CCUGAAGAACAUGACUGAA | UUCAGUCAUGUUCUUCAGG | | [399-417 |
| 175 | GGCUCUUAAGGCUUGAAGU | ACUUCAAGCCUUAAGAGCC | | [2313-2331] |
| 176 | UGGCUCUUAAGGCUUGAAG | CUUCAAGCCUUAAGAGCCA | | [2312-2330] |
| 177 | AACCUAGCAUCUUACAAAA | UUUUGUAAGAUGCUAGGUU | | [2225-2243] |
| 178 | UGUAAACAUUCAUCAAGCU | AGCUUGAUGAAUGUUUACA | | [2163-2181] |
| 179 | GAGAGAGUCUUCCAGGUAC | GUACCUGGAAGACUCUCUC | | [2096-2114] |
| 180 | ACGAGAGAGUCUUCCAGGU | ACCUGGAAGACUCUCUCGU | | [2094-2112] |
| 181 | UGGAUGGAAGUGUCUGGAA | UUCCAGACACUUCCAUCCA | | [2070-2088] |
| 182 | GGAAAAGUGGAUGGAAGUG | CACUUCCAUCCACUUUUCC | | [2063-2081] |
| 183 | GAGGAAAAGUGGAUGGAAG | CUUCCAUCCACUUUUCCUC | | [2061-2079] |
| 184 | AGAGGAAAAGUGGAUGGAA | UUCCAUCCACUUUUCCUCU | | [2060-2078] |
| 185 | ACAUUACAGUUUGGAAGUG | CACUUCCAAACUGUAAUGU | | [1923-1941] |
| 186 | CAACCUGUCUUCUCUCUGU | ACAGAGAGAAGACAGGUUG | | [194-212] |
| 187 | GAGAAGUCUAGGAUGUUUC | GAAACAUCCUAGACUUCUC | | [1823-1841 |
| 188 | CUCGGAGAGAAGUCUAGGA | UCCUAGACUUCUCUCCGAG | | [1817-1835 |
| 189 | AAGGGAAGGUGGGCAUUUU | AAAAUGCCCACCUUCCCUU | | [1751-1769 |
| 190 | GUGGAGGCACUCUUCAGUU | AACUGAAGAGUGCCUCCAC | | [1636-1654 |
| 191 | CUGGAACACACUAAGCUGA | UCAGCUUAGUGUGUUCCAG | | [1411-1429 |
| 192 | CCAGAAACAAAAAGAAGAG | CUCUUCUUUUUGUUUCUGG | | [1254-1272 |
| 193 | CAGGCCAGAAACAAAAAGA | UCUUUUUGUUUCUGGCCUG | | [1250-1268 |
| 194 | GUCUUGAAGAAGGGCAAUG | CAUUGCCCUUCUUCAAGAC | | [1057-1075 |
| 195 | AGGGAACAUUGUCUUUGUC | GACAAAGACAAUGUUCCCU | | [1041-1059 |
| 196 | GCCAGGGAACAUUGUCUUU | AAAGACAAUGUUCCCUGGC | | [1038-1056 |
| 197 | UGCCAGGGAACAUUGUCUU | AAGACAAUGUUCCCUGGCA | | [1037-1055 |
| 198 | CGUGUGCUAUUUGGGUUUG | CAAACCCAAAUAGCACACG | | [988-1006 |
| 199 | GGAAAAAAGCUGAAGAACA | UGUUCUUCAGCUUUUUUCC | | [662-680 |
| 200 | GCUAUCAAAGACCUUAAAG | CUUUAAGGUCUUUGAUAGC | Ms | [520-538 |
| 201 | CUCUUAAGGCUUGAAGUAA | UUACUUCAAGCCUUAAGAG | | [2315-2333] |
| 202 | GUAAUUGGCUCUUAAGGCU | AGCCUUAAGAGCCAAUUAC | | [2307-2325] |
| 203 | CUGAACCAUUACUGUAAUU | AAUUACAGUAAUGGUUCAG | | [2294-2312] |
| 204 | CCCAAGGAGUAAGUACAAA | UUUGUACUUACUCCUUGGG | | [2264-2282] |
| 205 | UCCUGUUUCUUGCUCUGAG | CUCAGAGCAAGAAACAGGA | | [2118-2136] |
| 206 | GGAGAGGAAAAGUGGAUGG | CCAUCCACUUUUCCUCUCC | | [2058-2076] |
| 207 | GGGAGAGGAAAAGUGGAUG | CAUCCACUUUUCCUCUCCC | | [2057-2075] |
| 208 | ACAGUUUGGAAGUGUUAAC | GUUAACACUUCCAAACUGU | | [1928-1946] |
| 209 | AGACAUUACAGUUUGGAAG | CUUCCAAACUGUAAUGUCU | | [1921-1939] |
| 210 | GAGACAUUACAGUUUGGAA | UUCCAAACUGUAAUGUCUC | | [1920-1938] |
| 211 | GAAAAUGAAGCCCUAUUAC | GUAAUAGGGCUUCAUUUUC | | [1860-1878] |
| 212 | GCUCCAACCUGUCUUCUCU | AGAGAAGACAGGUUGGAGC | | [190-208] |
| 213 | CCCUUUCAGAAGACAGCAU | AUGCUGUCUUCUGAAAGGG | | [1463-1481 |
| 214 | GAAGUUAUCUUAAGGGAGG | CCUCCCUUAAGAUAACUUC | | [95-113] |
| 215 | ACAAGUACACGGUAGUCAU | AUGACUACCGUGUACUUGU | | [1325-1343 |
| 216 | UGAGAAGUUAUCUUAAGGG | CCCUUAAGAUAACUUCUCA | | [92-110] |
| 217 | ACCAGGCCAGAAACAAAAA | UUUUUGUUUCUGGCCUGGU | | [1248-1266 |
| 218 | AUGGAAAAAAGCUGAAGAA | UUCUUCAGCUUUUUUCCAU | | [660-678 |
| 219 | GGAGGAAUGGAAAAAAGCU | AGCUUUUUUCCAUUCCUCC | | [654-672 |
| 220 | UCCCGGAUUUGCUUCAACA | UGUUGAAGCAAAUCCGGGA | | [361-379 |
| 221 | CUCCCGGAUUUGCUUCAAC | GUUGAAGCAAAUCCGGGAG | | [360-378 |
| 222 | ACUCCCGGAUUUGCUUCAA | UUGAAGCAAAUCCGGGAGU | | [359-377 |
| 223 | UUGGCUCUUAAGGCUUGAA | UUCAAGCCUUAAGAGCCAA | | [2311-2329] |
| 224 | UGAUCCUGUUUCUUGCUCU | AGAGCAAGAAACAGGAUCA | | [2115-2133] |
| 225 | GGUACUGAUCCUGUUUCUU | AAGAAACAGGAUCAGUACC | | [2110-2128] |
| 226 | GAGAGGAAAAGUGGAUGGA | UCCAUCCACUUUUCCUCUC | | [2059-2077] |
| 227 | CCUUCUGCUGUAUACUGUA | UACAGUAUACAGCAGAAGG | | [1899-1917] |
| 228 | CCAACCUGUCUUCUCUCUG | CAGAGAGAAGACAGGUUGG | | [193-211] |
| 229 | UCCAACCUGUCUUCUCUCU | AGAGAGAAGACAGGUUGGA | | [192-210] |
| 230 | AGCCCUAUUACUUGUUUGU | ACAAACAAGUAAUAGGGCU | | [1868-1886] |
| 231 | AUAUAAUCCUGGUGUUAUC | GAUAACACCAGGAUUAUAU | | [1697-1715 |
| 232 | AGACAACAGAACCUCAGCU | AGCUGAGGUUCUGUUGUCU | | [1601-1619 |
| 233 | UGAAAAAGCUGAUGCUAAU | AUUAGCAUCAGCUUUUUCA | | [1578-1596 |
| 234 | CCAUGCCCUACACACUCAA | UUGAGUGUGUAGGGCAUGG | | [1298-1316 |
| 235 | UGGAGUGUGUCUGGAAGCA | UGCUUCCAGACACACUCCA | | [740-758 |
| 236 | UGAGUCAGUUGCCAAAAGG | CCUUUUGGCAACUGACUCA | | [43-61] |
| 237 | GCUGAUAGACUAUAAGAUC | GAUCUUAUAGUCUAUCAGC | | [567-585 |
| 238 | CUGUUUCUGAACCAUUACU | AGUAAUGGUUCAGAAACAG | | [2288-2306] |
| 239 | CUGAUCCUGUUUCUUGCUC | GAGCAAGAAACAGGAUCAG | | [2114-2132] |
| 240 | CAGUUUGGAAGUGUUAACU | AGUUAACACUUCCAAACUG | | [1929-1947] |
| 241 | GGGACAUGGUGUCUAAGAA | UUCUUAGACACCAUGUCCC | | [1379-1397 |
| 242 | GCCAGAAACAAAAAGAAGA | UCUUCUUUUUGUUUCUGGC | | [1253-1271 |
| 243 | AGAAGGGCAAUGAUAACUG | CAGUUAUCAUUGCCCUUCU | | [1064-1082 |
| 244 | CUAUUUGGGUUUGUGCCUG | CAGGCACAAACCCAAAUAG | | [994-1012 |
| 245 | GAAUGGAAAAAAGCUGAAG | CUUCAGCUUUUUUCCAUUC | | [658-676 |
| 246 | GGUAGGGUUAUGAGUCAGU | ACUGACUCAUAACCCUACC | | [33-51] |
| 247 | CUCUCAUGGUAGGGUUAUG | CAUAACCCUACCAUGAGAG | | [26-44] |
| 248 | UCUCUCUCAUGGUAGGGUU | AACCCUACCAUGAGAGAGA | | [23-41] |
| 249 | UUGGCUGCAUGUACACUAU | AUAGUGUACAUGCAGCCAA | | [380-398 |
| 250 | GCCCAAGGAGUAAGUACAA | UUGUACUUACUCCUUGGGC | | [2263-2281] |
| 251 | CAGGUGAAUGGAGGAGUUA | UAACUCCUCCAUUCACCUG | | [2037-2055] |
| 252 | CAAAGCUGAAGAAAAUGAA | UUCAUUUUCUUCAGCUUUG | | [1850-1868] |
| 253 | CUUCUUUCCUUUUCUCUCU | AGAGAGAAAAGGAAAGAAG | | [11-29] |
| 254 | GAAACAAAAAGAAGAGCCU | AGGCUCUUCUUUUUGUUUC | | [1257-1275 |
| 255 | CUACCUUGAACCAGUUGAG | CUCAACUGGUUCAAGGUAG | GP | [1128-1146 |
| 256 | AGGAAUGGAAAAAAGCUGA | UCAGCUUUUUUCCAUUCCU | | [656-674 |
| 257 | GAGGAAUGGAAAAAAGCUG | CAGCUUUUUUCCAUUCCUC | | [655-673 |
| 258 | CUCUCUCAUGGUAGGGUUA | UAACCCUACCAUGAGAGAG | | [24-42] |
| 259 | CCUGUUUCUGAACCAUUAC | GUAAUGGUUCAGAAACAGG | | [2287-2305] |
| 260 | GGUGUUAUAUAACAUUGCU | AGCAAUGUUAUAUAACACC | | [618-636 |
| 261 | UGAACCAUUACUGUAAUUG | CAAUUACAGUAAUGGUUCA | | [2295-2313] |
| 262 | GCCUCCUAGUUUCUACCUA | UAGGUAGAAACUAGGAGGC | | [231-249] |
| 263 | GAAAAAGCUGAUGCUAAUA | UAUUAGCAUCAGCUUUUUC | | [1579-1597 |
| 264 | GCAGUGGCUUACUUCCAAC | GUUGGAAGUAAGCCACUGC | | [463-481 |
| 265 | GGCCCAAGGAGUAAGUACA | UGUACUUACUCCUUGGGCC | | [2262-2280] |
| 266 | UGAAGAAAAUGAAGCCCUA | UAGGGCUUCAUUUUCUUCA | | [1856-1874] |
| 267 | CGGUAGUCAUGAAGACUCA | UGAGUCUUCAUGACUACCG | | [1334-1352 |
| 268 | GAAGGAGGAAUGGAAAAAA | UUUUUUCCAUUCCUCCUUC | | [651-669 |
| 269 | GAGGUGUUAUAUAACAUUG | CAAUGUUAUAUAACACCUC | | [616-634 |
| 270 | ACCAGCUGAUAGACUAUAA | UUAUAGUCUAUCAGCUGGU | | [563-581 |
| 271 | CUCCUAGUUUCUACCUAAU | AUUAGGUAGAAACUAGGAG | | [233-251] |
| 272 | CUGUACAUUUGGUGCACUU | AAGUGCACCAAAUGUACAG | | [2180-2198] |
| 273 | GUGAAAAAGCUGAUGCUAA | UUAGCAUCAGCUUUUUCAC | | [1577-1595 |
| 274 | UGAGAACACAGUGGGUGAC | GUCACCCACUGUGUUCUCA | | [1530-1548 |
| 275 | UGAAAAACUACUGCCUGAC | GUCAGGCAGUAGUUUUUCA | Rat, Ms | [1502-1520 |
| 276 | CUCUGCUUCUUUCCUUUUC | GAAAAGGAAAGAAGCAGAG | | [6-24] |
| 277 | AGAAGGAGGAAUGGAAAAA | UUUUUCCAUUCCUCCUUCU | GP | [650-668 |
| 278 | GUUUGCCUGUGAGGUGUUA | UAACACCUCACAGGCAAAC | | [606-624 |
| 279 | GGCUAUCAAAGACCUUAAA | UUUAAGGUCUUUGAUAGCC | | [519-537 |
| 280 | CAAACUACAAAGCUGAAGA | UCUUCAGCUUUGUAGUUUG | | [1843-1861] |
| 281 | GGACCUGGAGUUUCAGGAA | UUCCUGAAACUCCAGGUCC | | [1674-1692 |
| 282 | GGACAUGGUGUCUAAGAAA | UUUCUUAGACACCAUGUCC | | [1380-1398 |
| 283 | UCCGGGACAUGGUGUCUAA | UUAGACACCAUGUCCCGGA | | [1376-1394 |
| 284 | ACACACUCAAGGUGCACUA | UAGUGCACCUUGAGUGUGU | GP | [1307-1325 |
| 285 | GCAACUACCUUGAACCAGU | ACUGGUUCAAGGUAGUUGC | GP | [1124-1142 |
| 286 | UGGAAGCAGAAGCUAUAUG | CAUAUAGCUUCUGCUUCCA | | [751-769 |
| 287 | CUGGAAGCAGAAGCUAUAU | AUAUAGCUUCUGCUUCCAG | | [750-768 |
| 288 | CCAAAAUCGACAAGGCGAU | AUCGCCUUGUCGAUUUUGG | | [722-740 |
| 289 | AGCCCAGACAUUCCAAAAU | AUUUUGGAAUGUCUGGGCU | | [710-728 |
| 290 | GCUUCAACAUUGGCUGCAU | AUGCAGCCAAUGUUGAAGC | | [371-389 |
| 291 | CUAGUUUCUACCUAAUCAU | AUGAUUAGGUAGAAACUAG | | [236-254] |
| 292 | UCCUAGUUUCUACCUAAUC | GAUUAGGUAGAAACUAGGA | | [234-252] |
| 293 | CCUAGCAUCUUACAAAAAC | GUUUUUGUAAGAUGCUAGG | | [2227-2245] |
| 294 | ACCUAGCAUCUUACAAAAA | UUUUUGUAAGAUGCUAGGU | | [2226-2244] |
| 295 | CUGUGUCAUACCACAAUAA | UUAUUGUGGUAUGACACAG | | [2201-2219] |
| 296 | GAAGAAAAUGAAGCCCUAU | AUAGGGCUUCAUUUUCUUC | | [1857-1875] |
| 297 | UCACAAACUACAAAGCUGA | UCAGCUUUGUAGUUUGUGA | | [1840-1858] |
| 298 | GGGAUAUAAUCCUGGUGUU | AACACCAGGAUUAUAUCCC | | [1694-1712 |
| 299 | UCCAGAUGAACCCAAGGAA | UUCCUUGGGUUCAUCUGGA | | [1557-1575 |
| 300 | GCCAGGUGAAAAACUACUG | CAGUAGUUUUUCACCUGGC | | [1496-1514 |
| 301 | CGGGACAUGGUGUCUAAGA | UCUUAGACACCAUGUCCCG | | [1378-1396 |
| 302 | CUGCUUCUUUCCUUUUCUC | GAGAAAAGGAAAGAAGCAG | | [8-26] |
| 303 | UCUGGAAGCAGAAGCUAUA | UAUAGCUUCUGCUUCCAGA | | [749-767 |
| 304 | AGGUGUUAUAUAACAUUGC | GCAAUGUUAUAUAACACCU | | [617-635 |
| 305 | CUAGCAUCUUACAAAAACA | UGUUUUUGUAAGAUGCUAG | | [2228-2246] |
| 306 | CUUUCCUUUUCUCUCUCAU | AUGAGAGAGAAAAGGAAAG | Rat | [14-32] |
| 307 | GCACUUUUCUGUGUCAUAC | GUAUGACACAGAAAAGUGC | | [2193-2211] |
| 308 | GCUGUGUUCACACUGUAAA | UUUACAGUGUGAACACAGC | | [2150-2168] |
| 309 | CCUGUCUUCUCUCUGUCUC | GAGACAGAGAGAAGACAGG | | [197-215] |
| 310 | CUGAAGAAAAUGAAGCCCU | AGGGCUUCAUUUUCUUCAG | | [1855-1873] |
| 311 | ACAAACUACAAAGCUGAAG | CUUCAGCUUUGUAGUUUGU | | [1842-1860] |
| 312 | CACAAACUACAAAGCUGAA | UUCAGCUUUGUAGUUUGUG | | [1841-1859] |
| 313 | GCACUCGGAGAGAAGUCUA | UAGACUUCUCUCCGAGUGC | | [1814-1832 |
| 314 | GACUGCGCAACUACAGAUU | AAUCUGUAGUUGCGCAGUC | | [1789-1807 |
| 315 | AGACUGCGCAACUACAGAU | AUCUGUAGUUGCGCAGUCU | | [1788-1806 |
| 316 | CCAGGUGAAAAACUACUGC | GCAGUAGUUUUUCACCUGG | | [1497-1515 |
| 317 | GCAACACUGAGAAGUUAUC | GAUAACUUCUCAGUGUUGC | | [85-103] |
| 318 | UCUGCUUCUUUCCUUUUCU | AGAAAAGGAAAGAAGCAGA | | [7-25] |
| 319 | AGAAGGAUUACCUAGGCAA | UUGCCUAGGUAAUCCUUCU | | [836-854 |
| 320 | CAAGAAGGAUUACCUAGGC | GCCUAGGUAAUCCUUCUUG | | [834-852 |
| 321 | GAAGCUAUAUGAGCCAGUG | CACUGGCUCAUAUAGCUUC | | [759-777 |
| 322 | AGAAGCUAUAUGAGCCAGU | ACUGGCUCAUAUAGCUUCU | | [758-776 |
| 323 | GCAGAAGCUAUAUGAGCCA | UGGCUCAUAUAGCUUCUGC | | [756-774 |
| 324 | GUGAGGUGUUAUAUAACAU | AUGUUAUAUAACACCUCAC | | [614-632 |
| 325 | CCGGAUUUGCUUCAACAUU | AAUGUUGAAGCAAAUCCGG | | [363-381 |
| 326 | CAGCUGUGUUCACACUGUA | UACAGUGUGAACACAGCUG | | [2148-2166] |
| 327 | CCCUGUUAAAAUUUAACCU | AGGUUAAAUUUUAACAGGG | | [1955-1973] |
| 328 | CCCUAUUACUUGUUUGUAA | UUACAAACAAGUAAUAGGG | | [1870-1888] |
| 329 | ACAGCAUGAAGGAUGCCUG | CAGGCAUCCUUCAUGCUGU | | [1475-1493 |
| 330 | GUACACGGUAGUCAUGAAG | CUUCAUGACUACCGUGUAC | | [1329-1347 |
| 331 | AGUACACGGUAGUCAUGAA | UUCAUGACUACCGUGUACU | | [1328-1346 |
| 332 | AGACAGUUUCUCUGGGUUU | AAACCCAGAGAAACUGUCU | | [885-903 |
| 333 | AGCUGGCCAAGAAGGAUUA | UAAUCCUUCUUGGCCAGCU | | [827-845 |
| 334 | UUGCUUUCAUGUAUGCCAA | UUGGCAUACAUGAAAGCAA | | [632-650 |
| 335 | CCAGCUGAUAGACUAUAAG | CUUAUAGUCUAUCAGCUGG | | [564-582 |
| 336 | AACCAGCUGAUAGACUAUA | UAUAGUCUAUCAGCUGGUU | | [562-580 |
| 337 | UCUGUGUCAUACCACAAUA | UAUUGUGGUAUGACACAGA | | [2200-2218] |
| 338 | GUGCACUUUUCUGUGUCAU | AUGACACAGAAAAGUGCAC | | [2191-2209] |
| 339 | GUACAUUUGGUGCACUUUU | AAAAGUGCACCAAAUGUAC | | [2182-2200] |
| 340 | CUCUUGGCCUCCUAGUUUC | GAAACUAGGAGGCCAAGAG | | [225-243] |
| 341 | UCCAGGUACUGAUCCUGUU | AACAGGAUCAGUACCUGGA | | [2106-2124] |
| 342 | UCUUUCCUUUUCUCUCUCA | UGAGAGAGAAAAGGAAAGA | Rat | [13-31] |
| 343 | GACCUGGAGUUUCAGGAAG | CUUCCUGAAACUCCAGGUC | | [1675-1693 |
| 344 | AGAACCUCAGCUUAAGAAA | UUUCUUAAGCUGAGGUUCU | | [1608-1626 |
| 345 | AGAACACAGUGGGUGACCA | UGGUCACCCACUGUGUUCU | | [1532-1550 |
| 346 | UGGUGUCUAAGAAACUGGA | UCCAGUUUCUUAGACACCA | | [1385-1403 |
| 347 | GGUUUGUGCCUGAGACAAA | UUUGUCUCAGGCACAAACC | | [1001-1019 |
| 348 | CUGAUGCAUUUGCAACACU | AGUGUUGCAAAUGCAUCAG | | [74-92] |
| 349 | GGCUCACCGUGUGCUAUUU | AAAUAGCACACGGUGAGCC | | [981-999 |
| 350 | CCUGAUGCAUUUGCAACAC | GUGUUGCAAAUGCAUCAGG | | [73-91] |
| 351 | GGCCAAGAAGGAUUACCUA | UAGGUAAUCCUUCUUGGCC | | [831-849 |
| 352 | UGAGAGACAAGUGGCUCAG | CUGAGCCACUUGUCUCUCA | | [810-828 |
| 353 | GACAUUUCCUGAUGCAUUU | AAAUGCAUCAGGAAAUGUC | | [66-84] |
| 354 | GGACAUUUCCUGAUGCAUU | AAUGCAUCAGGAAAUGUCC | | [65-83] |
| 355 | GCCCAGACAUUCCAAAAUC | GAUUUUGGAAUGUCUGGGC | | [711-729 |
| 356 | GAAGUCUGAGCCCAGACAU | AUGUCUGGGCUCAGACUUC | | [702-720 |
| 357 | CAGUUAGCAUUGGCCACGA | UCGUGGCCAAUGCUAACUG | | [679-697 |
| 358 | GCCUGUGAGGUGUUAUAUA | UAUAUAACACCUCACAGGC | | [610-628 |
| 359 | GAAAUAUGAUUUGGCUAUC | GAUAGCCAAAUCAUAUUUC | | [507-525 |
| 360 | CAACGAGGGAUGCUCUACU | AGUAGAGCAUCCCUCGUUG | | [478-496 |
| 361 | GAAGUAACCUUAUAGGUUA | UAACCUAUAAGGUUACUUC | | [2327-2345] |
| 362 | UCUGAACCAUUACUGUAAU | AUUACAGUAAUGGUUCAGA | | [2293-2311] |
| 363 | GUGUCAUACCACAAUAAAA | UUUUAUUGUGGUAUGACAC | | [2203-2221] |
| 364 | CUCUCUUGGCCUCCUAGUU | AACUAGGAGGCCAAGAGAG | | [223-241] |
| 365 | UGAAGCCCUAUUACUUGUU | AACAAGUAAUAGGGCUUCA | | [1865-1883] |
| 366 | GCACUCUUCAGUUAUGAGG | CCUCAUAACUGAAGAGUGC | | [1642-1660 |
| 367 | CUGAUGCUAAUAACCAGAC | GUCUGGUUAUUAGCAUCAG | | [1586-1604 |
| 368 | GCUGAUGCUAAUAACCAGA | UCUGGUUAUUAGCAUCAGC | | [1585-1603 |
| 369 | CCUUUCAGAAGACAGCAUG | CAUGCUGUCUUCUGAAAGG | | [1464-1482 |
| 370 | UUGCAACACUGAGAAGUUA | UAACUUCUCAGUGUUGCAA | | [83-101] |
| 371 | UGAGACAAAAGAAGAGCUC | GAGCUCUUCUUUUGUCUCA | | [1011-1029 |
| 372 | UGAUGCAUUUGCAACACUG | CAGUGUUGCAAAUGCAUCA | | [75-93] |
| 373 | GUGUCUGGAAGCAGAAGCU | AGCUUCUGCUUCCAGACAC | | [746-764 |
| 374 | CCAGACAUUCCAAAAUCGA | UCGAUUUUGGAAUGUCUGG | | [713-731 |
| 375 | UGCCUGUGAGGUGUUAUAU | AUAUAACACCUCACAGGCA | | [609-627 |
| 376 | GGCUCCAGUUCAAGCUGUU | AACAGCUUGAACUGGAGCC | | [590-608 |
| 377 | AGCCUUGAUUCAGCUUCGA | UCGAAGCUGAAUCAAGGCU | | [540-558 |
| 378 | AGACCUUAAAGAAGCCUUG | CAAGGCUUCUUUAAGGUCU | | [528-546 |
| 379 | UGCUCUACUACCAGACAGA | UCUGUCUGGUAGUAGAGCA | | [488-506 |
| 380 | CCUUUACCAGAAGCAUUAA | UUAAUGCUUCUGGUAAAGG | | [428-446 |
| 381 | CUAUCCUGAAGAACAUGAC | GUCAUGUUCUUCAGGAUAG | | [395-413 |
| 382 | ACCUUAUAGGUUACUCAUA | UAUGAGUAACCUAUAAGGU | | [2333-2351] |
| 383 | UUCUGUGUCAUACCACAAU | AUUGUGGUAUGACACAGAA | | [2199-2217] |
| 384 | UGCACUUUUCUGUGUCAUA | UAUGACACAGAAAAGUGCA | | [2192-2210] |
| 385 | GGUGCACUUUUCUGUGUCA | UGACACAGAAAAGUGCACC | | [2190-2208] |
| 386 | CCAGCUGUGUUCACACUGU | ACAGUGUGAACACAGCUGG | | [2147-2165] |
| 387 | CUAGGAUGUUUCACAAACU | AGUUUGUGAAACAUCCUAG | | [1830-1848 |
| 388 | CCCAGAAAGUGAACACCUU | AAGGUGUUCACUUUCUGGG | | [138-156] |
| 389 | UGUUAUCAAAGGUGAAUGA | UCAUUCACCUUUGAUAACA | | [1709-1727 |
| 390 | GGAUAUAAUCCUGGUGUUA | UAACACCAGGAUUAUAUCC | | [1695-1713 |
| 391 | GGCACUCUUCAGUUAUGAG | CUCAUAACUGAAGAGUGCC | | [1641-1659 |
| 392 | AGCUGAUGCUAAUAACCAG | CUGGUUAUUAGCAUCAGCU | | [1584-1602 |
| 393 | ACCAAGGCUUUCCAGAUGA | UCAUCUGGAAAGCCUUGGU | | [1547-1565 |
| 394 | GACAUGGUGUCUAAGAAAC | GUUUCUUAGACACCAUGUC | | [1381-1399 |
| 395 | GCACUACAAGUACACGGUA | UACCGUGUACUUGUAGUGC | | [1320-1338 |
| 396 | CCUACACACUCAAGGUGCA | UGCACCUUGAGUGUGUAGG | | [1304-1322 |
| 397 | GCUCCUCCUAGUUCCAAAG | CUUUGGAACUAGGAGGAGC | | [1207-1225 |
| 398 | UGCAACACUGAGAAGUUAU | AUAACUUCUCAGUGUUGCA | | [84-102] |
| 399 | AAGACAGUUUCUCUGGGUU | AACCCAGAGAAACUGUCUU | | [884-902 |
| 400 | CCAAGAAGGAUUACCUAGG | CCUAGGUAAUCCUUCUUGG | | [833-851 |
| 401 | CCAAAUGAGAGACAAGUGG | CCACUUGUCUCUCAUUUGG | | [805-823 |
| 402 | AAAAGCUGAAGAACAGUUA | UAACUGUUCUUCAGCUUUU | | [666-684 |
| 403 | UUAUGAGUCAGUUGCCAAA | UUUGGCAACUGACUCAUAA | | [40-58] |
| 404 | UUGCCUGUGAGGUGUUAUA | UAUAACACCUCACAGGCAA | | [608-626 |
| 405 | GCCUUGAUUCAGCUUCGAG | CUCGAAGCUGAAUCAAGGC | | [541-559 |
| 406 | UGUGUCAUACCACAAUAAA | UUUAUUGUGGUAUGACACA | | [2202-2220] |
| 407 | AGCUGUGUUCACACUGUAA | UUACAGUGUGAACACAGCU | | [2149-2167] |
| 408 | CCAGGUACUGAUCCUGUUU | AAACAGGAUCAGUACCUGG | | [2107-2125] |
| 409 | AGACGCUCCAACCUGUCUU | AAGACAGGUUGGAGCGUCU | | [186-204] |
| 410 | UCAGUUAUGAGGCUACCCA | UGGGUAGCCUCAUAACUGA | | [1649-1667 |
| 411 | UGACCAAGGCUUUCCAGAU | AUCUGGAAAGCCUUGGUCA | | [1545-1563 |
| 412 | UCCGGCUGGAACACACUAA | UUAGUGUGUUCCAGCCGGA | | [1406-1424 |
| 413 | AGCCUAAGGAAGUGAAGCU | AGCUUCACUUCCUUAGGCU | | [1271-1289 |
| 414 | CCUUGAACCAGUUGAGCUG | CAGCUCAACUGGUUCAAGG | | [1131-1149 |
| 415 | CAAGACAGUUUCUCUGGGU | ACCCAGAGAAACUGUCUUG | | [883-901 |
| 416 | UCCUGAUGCAUUUGCAACA | UGUUGCAAAUGCAUCAGGA | | [72-90] |
| 417 | GGAUCAAGACAGUUUCUCU | AGAGAAACUGUCUUGAUCC | | [879-897 |
| 418 | AGAGACAAGUGGCUCAGCU | AGCUGAGCCACUUGUCUCU | GP | [812-830 |
| 419 | CUGAAGAACAGUUAGCAUU | AAUGCUAACUGUUCUUCAG | | [671-689 |
| 420 | AAAAAGCUGAAGAACAGUU | AACUGUUCUUCAGCUUUUU | | [665-683 |
| 421 | CUUUCAUGUAUGCCAAGAA | UUCUUGGCAUACAUGAAAG | | [635-653 |
| 422 | ACAUUGCUUUCAUGUAUGC | GCAUACAUGAAAGCAAUGU | | [629-647 |
| 423 | UGUGAGGUGUUAUAUAACA | UGUUAUAUAACACCUCACA | | [613-631 |
| 424 | CUCCAGUUCAAGCUGUUUG | CAAACAGCUUGAACUGGAG | | [592-610 |
| 425 | GAACCAGCUGAUAGACUAU | AUAGUCUAUCAGCUGGUUC | | [561-579 |
| 426 | GAAGCCUUGAUUCAGCUUC | GAAGCUGAAUCAAGGCUUC | | [538-556 |
| 427 | AGAAGCCUUGAUUCAGCUU | AAGCUGAAUCAAGGCUUCU | | [537-555 |
| 428 | GGAUGCUCUACUACCAGAC | GUCUGGUAGUAGAGCAUCC | | [485-503 |
| 429 | GGGAUGCUCUACUACCAGA | UCUGGUAGUAGAGCAUCCC | | [484-502 |
| 430 | GUGGCUUACUUCCAACGAG | CUCGUUGGAAGUAAGCCAC | GP | [466-484 |
| 431 | UGCUUCAACAUUGGCUGCA | UGCAGCCAAUGUUGAAGCA | | [370-388 |
| 432 | CUUAUAGGUUACUCAUAAG | CUUAUGAGUAACCUAUAAG | | [2335-2353] |
| 433 | AGGCUUGAAGUAACCUUAU | AUAAGGUUACUUCAAGCCU | | [2321-2339] |
| 434 | UUCUGAACCAUUACUGUAA | UUACAGUAAUGGUUCAGAA | | [2292-2310] |
| 435 | GCAUCUUACAAAAACAAGA | UCUUGUUUUUGUAAGAUGC | | [2231-2249] |
| 436 | GCUGUACAUUUGGUGCACU | AGUGCACCAAAUGUACAGC | | [2179-2197] |
| 437 | UACUGAGACAUUACAGUUU | AAACUGUAAUGUCUCAGUA | | [1916-1934] |
| 438 | GUAAGAUUUAGCACCCUUC | GAAGGGUGCUAAAUCUUAC | | [1885-1903] |
| 439 | UAGGAUGUUUCACAAACUA | UAGUUUGUGAAACAUCCUA | | [1831-1849 |
| 440 | CAGUUAUGAGGCUACCCAA | UUGGGUAGCCUCAUAACUG | | [1650-1668 |
| 441 | CUACUCAUCUGGCCCAGAA | UUCUGGGCCAGAUGAGUAG | | [126-144] |
| 442 | ACGGUAGUCAUGAAGACUC | GAGUCUUCAUGACUACCGU | | [1333-1351 |
| 443 | ACACGGUAGUCAUGAAGAC | GUCUUCAUGACUACCGUGU | | [1331-1349 |
| 444 | GGCAAUGAUAACUGGGCCA | UGGCCCAGUUAUCAUUGCC | | [1069-1087 |
| 445 | UGCAUUUGCAACACUGAGA | UCUCAGUGUUGCAAAUGCA | | [78-96] |
| 446 | GUGCCUGAGACAAAAGAAG | CUUCUUUUGUCUCAGGCAC | | [1006-1024 |
| 447 | AGCUAUAUGAGCCAGUGGU | ACCACUGGCUCAUAUAGCU | | [761-779 |
| 448 | GGGACAUUUCCUGAUGCAU | AUGCAUCAGGAAAUGUCCC | | [64-82] |
| 449 | UGAAGUCUGAGCCCAGACA | UGUCUGGGCUCAGACUUCA | | [701-719 |
| 450 | AGCUGAAGAACAGUUAGCA | UGCUAACUGUUCUUCAGCU | | [669-687 |
| 451 | GCUUUCAUGUAUGCCAAGA | UCUUGGCAUACAUGAAAGC | | [634-652 |
| 452 | UCAACAUUGGCUGCAUGUA | UACAUGCAGCCAAUGUUGA | | [374-392 |
| 453 | CCCGGAUUUGCUUCAACAU | AUGUUGAAGCAAAUCCGGG | | [362-380 |
| 454 | GUACCCAGGAUGAUUUCCU | AGGAAAUCAUCCUGGGUAC | | [1991-2009] |
| 455 | ACUGAGACAUUACAGUUUG | CAAACUGUAAUGUCUCAGU | | [1917-1935] |
| 456 | GUACUGAGACAUUACAGUU | AACUGUAAUGUCUCAGUAC | | [1915-1933] |
| 457 | CUGCGCAACUACAGAUUUG | CAAAUCUGUAGUUGCGCAG | | [1791-1809 |
| 458 | UCCUGGUGUUAUCAAAGGU | ACCUUUGAUAACACCAGGA | | [1703-1721 |
| 459 | CCUCAGCUUAAGAAAGGCA | UGCCUUUCUUAAGCUGAGG | | [1612-1630 |
| 460 | GAAGCUCAGUGUUCCCAUG | CAUGGGAACACUGAGCUUC | | [1284-1302 |
| 461 | GGGUUUGUGCCUGAGACAA | UUGUCUCAGGCACAAACCC | | [1000-1018 |
| 462 | UGGAUCAAGACAGUUUCUC | GAGAAACUGUCUUGAUCCA | | [878-896 |
| 463 | GCCAAGAAGGAUUACCUAG | CUAGGUAAUCCUUCUUGGC | | [832-850 |
| 464 | UGGCCAAGAAGGAUUACCU | AGGUAAUCCUUCUUGGCCA | | [830-848 |
| 465 | UGAGCCCAGACAUUCCAAA | UUUGGAAUGUCUGGGCUCA | | [708-726 |
| 466 | GGCCUUUACCAGAAGCAUU | AAUGCUUCUGGUAAAGGCC | | [426-444 |
| 467 | GUAACCUUAUAGGUUACUC | GAGUAACCUAUAAGGUUAC | | [2330-2348] |
| 468 | UGAAGUAACCUUAUAGGUU | AACCUAUAAGGUUACUUCA | | [2326-2344] |
| 469 | UGGUGCACUUUUCUGUGUC | GACACAGAAAAGUGCACCA | | [2189-2207] |
| 470 | UGUUCACACUGUAAACAUU | AAUGUUUACAGUGUGAACA | | [2154-2172] |
| 471 | AGGUACUGAUCCUGUUUCU | AGAAACAGGAUCAGUACCU | | [2109-2127] |
| 472 | UUAGCACCCUUCUGCUGUA | UACAGCAGAAGGGUGCUAA | | [1892-1910] |
| 473 | CCAUUCUACUCAUCUGGCC | GGCCAGAUGAGUAGAAUGG | | [121-139] |
| 474 | UGAAGCUCAGUGUUCCCAU | AUGGGAACACUGAGCUUCA | | [1283-1301 |
| 475 | CUAAGGAAGUGAAGCUCAG | CUGAGCUUCACUUCCUUAG | | [1274-1292 |
| 476 | GGCUUGUUCCCUGCAACUA | UAGUUGCAGGGAACAAGCC | GP | [1112-1130 |
| 477 | AGGCUCACCGUGUGCUAUU | AAUAGCACACGGUGAGCCU | | [980-998 |
| 478 | AGACAUUCCAAAAUCGACA | UGUCGAUUUUGGAAUGUCU | | [715-733 |
| 479 | CCCAGACAUUCCAAAAUCG | CGAUUUUGGAAUGUCUGGG | | [712-730 |
| 480 | GCUGAAGAACAGUUAGCAU | AUGCUAACUGUUCUUCAGC | | [670-688 |
| 481 | UGAUAGACUAUAAGAUCCU | AGGAUCUUAUAGUCUAUCA | GP | [569-587 |
| 482 | AAGGCUUGAAGUAACCUUA | UAAGGUUACUUCAAGCCUU | | [2320-2338] |
| 483 | UAAGGCUUGAAGUAACCUU | AAGGUUACUUCAAGCCUUA | | [2319-2337] |
| 484 | CCAUUACUGUAAUUGGCUC | GAGCCAAUUACAGUAAUGG | | [2299-2317] |
| 485 | AGCUGUACAUUUGGUGCAC | GUGCACCAAAUGUACAGCU | | [2178-2196] |
| 486 | GUGUUCACACUGUAAACAU | AUGUUUACAGUGUGAACAC | | [2153-2171] |
| 487 | CAGGUACUGAUCCUGUUUC | GAAACAGGAUCAGUACCUG | | [2108-2126] |
| 488 | GAGUCUUCCAGGUACUGAU | AUCAGUACCUGGAAGACUC | | [2100-2118] |
| 489 | CCUAUUACUUGUUUGUAAG | CUUACAAACAAGUAAUAGG | | [1871-1889] |
| 490 | GUCUAAGAAACUGGAGCUC | GAGCUCCAGUUUCUUAGAC | | [1389-1407 |
| 491 | CACUACAAGUACACGGUAG | CUACCGUGUACUUGUAGUG | | [1321-1339 |
| 492 | UGCACUACAAGUACACGGU | ACCGUGUACUUGUAGUGCA | | [1319-1337 |
| 493 | AGGUGCACUACAAGUACAC | GUGUACUUGUAGUGCACCU | GP | [1316-1334 |
| 494 | UCAGUGUUCCCAUGCCCUA | UAGGGCAUGGGAACACUGA | | [1289-1307 |
| 495 | CAACUACCUUGAACCAGUU | AACUGGUUCAAGGUAGUUG | GP | [1125-1143 |
| 496 | GUUUGUGCCUGAGACAAAA | UUUUGUCUCAGGCACAAAC | | [1002-1020 |
| 497 | GAGCCCAGACAUUCCAAAA | UUUUGGAAUGUCUGGGCUC | | [709-727 |
| 498 | GAAAAAAGCUGAAGAACAG | CUGUUCUUCAGCUUUUUUC | | [663-681 |
| 499 | CAAGCUGUUUGCCUGUGAG | CUCACAGGCAAACAGCUUG | Ms | [600-618 |
| 500 | UCUACUACCAGACAGAGAA | UUCUCUGUCUGGUAGUAGA | | [491-509 |
| 501 | GAAGCAGAGAAGGCCUUUA | UAAAGGCCUUCUCUGCUUC | | [415-433 |
| 502 | AGCCAGCUGUGUUCACACU | AGUGUGAACACAGCUGGCU | | [2145-2163] |
| 503 | CUAGCUAGCCAGCUGUGUU | AACACAGCUGGCUAGCUAG | | [2139-2157] |
| 504 | UGACUCUGUGGUGUGAGAA | UUCUCACACCACAGAGUCA | | [1517-1535 |
| 505 | GAGUGUGUCUGGAAGCAGA | UCUGCUUCCAGACACACUC | | [742-760 |
| 506 | GGUUAUGAGUCAGUUGCCA | UGGCAACUGACUCAUAACC | | [38-56] |
| 507 | GCUCCAGUUCAAGCUGUUU | AAACAGCUUGAACUGGAGC | | [591-609 |
| 508 | CUGAAGCAGAGAAGGCCUU | AAGGCCUUCUCUGCUUCAG | | [413-431 |
| 509 | UGUAAGAUUUAGCACCCUU | AAGGGUGCUAAAUCUUACA | | [1884-1902] |
| 510 | CCAGAAAGUGAACACCUUG | CAAGGUGUUCACUUUCUGG | | [139-157] |
| 511 | GCCCAGAAAGUGAACACCU | AGGUGUUCACUUUCUGGGC | | [137-155] |
| 512 | UGGCCCAGAAAGUGAACAC | GUGUUCACUUUCUGGGCCA | | [135-153] |
| 513 | GCCAAGAAGGAGGAAUGGA | UCCAUUCCUCCUUCUUGGC | GP | [646-664 |
| 514 | AGCACCCUUCUGCUGUAUA | UAUACAGCAGAAGGGUGCU | | [1894-1912] |
| 515 | CAGCUGGCCAAGAAGGAUU | AAUCCUUCUUGGCCAGCUG | | [826-844 |
| 516 | AGGCCUUUACCAGAAGCAU | AUGCUUCUGGUAAAGGCCU | | [425-443 |
| 517 | CUAGCCAGCUGUGUUCACA | UGUGAACACAGCUGGCUAG | | [2143-2161] |
| 518 | UGCUCUGAGUGCUAGCUAG | CUAGCUAGCACUCAGAGCA | | [2128-2146] |
| 519 | UGGCUAUCAAAGACCUUAA | UUAAGGUCUUUGAUAGCCA | | [518-536 |
| 520 | UAGCACCCUUCUGCUGUAU | AUACAGCAGAAGGGUGCUA | | [1893-1911] |
| 521 | GCUUAAGAAAGGCAGCCAA | UUGGCUGCCUUUCUUAAGC | | [1617-1635 |
| 522 | CUGACUCUGUGGUGUGAGA | UCUCACACCACAGAGUCAG | | [1516-1534 |
| 523 | UGAUUUGGCUAUCAAAGAC | GUCUUUGAUAGCCAAAUCA | | [513-531 |
| 524 | AGAAGGCCUUUACCAGAAG | CUUCUGGUAAAGGCCUUCU | | [422-440 |
| 525 | GGUCAUGCCAGGGAACAUU | AAUGUUCCCUGGCAUGACC | | [1032-1050 |
| 526 | AGCUUAAGAAAGGCAGCCA | UGGCUGCCUUUCUUAAGCU | | [1616-1634 |

TABLE A18

NOXA2 19-mer oligomer pairs (NCF2, neutrophil
cytosolic factor 2, p67phox)

| | | | | |
|---|---|---|---|---|
| | | | | Human-67189969 |
| No. | Sense siRNA | AntiSense siRNA | Other Sp | ORF: 253-1833 |

| | | | | |
|---|---|---|---|---|
| 1 | CCACGAGCAUGAAGUCUGA | UCAGACUUCAUGCUCGUGG | | [692-710] |
| 2 | GCAUGUACACUAUCCUGAA | UUCAGGAUAGUGUACAUGC | | [386-404] |
| 3 | CCUCCUAGUUUCUACCUAA | UUAGGUAGAAACUAGGAGG | | [232-250] |
| 4 | CUACAAAGCUGAAGAAAAU | AUUUUCUUCAGCUUUGUAG | | [1847-1865] |
| 5 | GAGGGAACCAGCUGAUAGA | UCUAUCAGCUGGUUCCCUC | | [557-575] |
| 6 | CCAGAUGAACCCAAGGAAA | UUUCCUUGGGUUCAUCUGG | | [1558-1576] |
| 7 | GAUGUUUCACAAACUACAA | UUGUAGUUUGUGAAACAUC | | [1834-1852] |
| 8 | GGAUGGAAGUGUCUGGAAA | UUUCCAGACACUUCCAUCC | | [2071-2089] |
| 9 | GAGGGAUGCUCUACUACCA | UGGUAGUAGAGCAUCCCUC | | [482-500] |
| 10 | GCAACUACAGAUUUGGAAA | UUUCCAAAUCUGUAGUUGC | | [1795-1813] |
| 11 | CAAGAAGGAGGAAUGGAAA | UUUCCAUUCCUCCUUCUUG | GP | [648-666] |
| 12 | AAAAGAAGAGCCUAAGGAA | UUCCUUAGGCUCUUCUUUU | Rat | [1263-1281] |
| 13 | GGCUUGAAGUAACCUUAUA | UAUAAGGUUACUUCAAGCC | | [2322-2340] |
| 14 | CCAAGGCUUUCCAGAUGAA | UUCAUCUGGAAAGCCUUGG | | [1548-1566] |
| 15 | CCUGUGAGGUGUUAUAUAA | UUAUAUAACACCUCACAGG | | [611-629] |
| 16 | CCAAGAAGGAGGAAUGGAA | UUCCAUUCCUCCUUCUUGG | GP | [647-665] |
| 17 | CAGCUGAUAGACUAUAAGA | UCUUAUAGUCUAUCAGCUG | | [565-583] |
| 18 | ACACUAUCCUGAAGAACAU | AUGUUCUUCAGGAUAGUGU | | [392-410] |
| 19 | UAAGGCAUAUACAAAUAAA | UUUAUUUGUAUAUGCCUUA | | [2350-2368] |
| 20 | GGAACCAGCUGAUAGACUA | UAGUCUAUCAGCUGGUUCC | Ms | [560-578] |
| 21 | GUCAUACCACAAUAAAAAA | UUUUUUAUUGUGGUAUGAC | | [2205-2223] |
| 22 | CAAGGAGUAAGUACAAAUA | UAUUUGUACUUACUCCUUG | | [2266-2284] |
| 23 | CAAGCUGUUUCGACCAAAU | AUUUGGUCGAAACAGCUUG | | [792-810] |
| 24 | CCUAGUUUCUACCUAAUCA | UGAUUAGGUAGAAACUAGG | | [235-253] |
| 25 | UGAAGAAGGGCAAUGAUAA | UUAUCAUUGCCCUUCUUCA | | [1061-1079] |
| 26 | GAACCCAAGGAAAGUGAAA | UUUCACUUUCCUUGGGUUC | GP | [1564-1582] |
| 27 | GCUGUAUACUGUACUGAGA | UCUCAGUACAGUAUACAGC | | [1905-1923] |
| 28 | UGUCAUACCACAAUAAAAA | UUUUUAUUGUGGUAUGACA | | [2204-2222] |
| 29 | CAUACCACAAUAAAAAAAA | UUUUUUUUAUUGUGGUAUG | | [2207-2225] |
| 30 | CAGAGGACCUGGAGUUUCA | UGAAACUCCAGGUCCUCUG | | [1670-1688] |
| 31 | CUAUUUAUUCCCUGUUAAA | UUUAACAGGGAAUAAAUAG | | [1946-1964] |
| 32 | CAUCUGUGGUGGAUCAAGA | UCUUGAUCCACCACAGAUG | GP | [869-887] |
| 33 | ACUCAUAAGGCAUAUACAA | UUGUAUAUGCCUUAUGAGU | | [2345-2363] |
| 34 | AAACAUUCAUCAAGCUGUA | UACAGCUUGAUGAAUGUUU | | [2166-2184] |
| 35 | AAAUAUUCCUGUUUCUGAA | UUCAGAAACAGGAAUAUUU | | [2280-2298] |
| 36 | ACUACAAAGCUGAAGAAAA | UUUUCUUCAGCUUUGUAGU | | [1846-1864] |
| 37 | UGAAGACUGCGCAACUACA | UGUAGUUGCGCAGUCUUCA | | [1785-1803] |
| 38 | CCAUCAGCCUCUGGAAUGA | UCAUUCCAGAGGCUGAUGG | | [269-287] |
| 39 | CUGUUUCGACCAAAUGAGA | UCUCAUUUGGUCGAAACAG | | [796-814] |
| 40 | CAUAAGGCAUAUACAAAUA | UAUUUGUAUAUGCCUUAUG | | [2348-2366] |
| 41 | UAAUAACCAGACAACAGAA | UUCUGUUGUCUGGUUAUUA | | [1593-1611] |
| 42 | CAGAAGACAGCAUGAAGGA | UCCUUCAUGCUGUCUUCUG | | [1469-1487] |
| 43 | UGUGCCUGAGACAAAAGAA | UUCUUUUGUCUCAGGCACA | | [1005-1023] |
| 44 | UAUCAAAGGUGAAUGAAGA | UCUUCAUUCACCUUUGAUA | | [1712-1730] |
| 45 | GGAAGCAGAAGCUAUAUGA | UCAUAUAGCUUCUGCUUCC | | [752-770] |
| 46 | GGAAGUGUUAACUAUUUAU | AUAAAUAGUUAACACUUCC | | [1935-1953] |
| 47 | AAACUACAAAGCUGAAGAA | UUCUUCAGCUUUGUAGUUU | | [1844-1862] |
| 48 | CAUGAAGUCUGAGCCCAGA | UCUGGGCUCAGACUUCAUG | | [699-717] |
| 49 | AAUUUAACCUACUAGACAA | UUGUCUAGUAGGUUAAAUU | | [1964-1982] |
| 50 | GGAAAGCACUCGGAGAGAA | UUCUCUCCGAGUGCUUUCC | | [1809-1827] |
| 51 | GAUCUUCAGGGCUCUGGAA | UUCCAGAGCCCUGAAGAUC | Rat | [957-975] |
| 52 | CAUCAGCCUCUGGAAUGAA | UUCAUUCCAGAGGCUGAUG | | [270-288] |
| 53 | AGGAGUAAGUACAAAUAUU | AAUAUUUGUACUUACUCCU | | [2268-2286] |
| 54 | CAGGGAACAUUGUCUUUGU | ACAAAGACAAUGUUCCCUG | | [1040-1058] |
| 55 | CUUGAAGAAGGGCAAUGAU | AUCAUUGCCCUUCUUCAAG | | [1059-1077] |
| 56 | UAUCAAAGACCUUAAAGAA | UUCUUUAAGGUCUUUGAUA | | [522-540] |
| 57 | GCAAGCUGUUUCGACCAAA | UUUGGUCGAAACAGCUUGC | | [791-809] |
| 58 | CAACAGAACCUCAGCUUAA | UUAAGCUGAGGUUCUGUUG | | [1604-1622] |
| 59 | AUCAAAGGUGAAUGAAGAA | UUCUUCAUUCACCUUUGAU | | [1713-1731] |
| 60 | GAUGCAUUUGCAACACUGA | UCAGUGUUGCAAAUGCAUC | | [76-94] |
| 61 | AGAAGUCUAGGAUGUUUCA | UGAAACAUCCUAGACUUCU | | [1824-1842] |
| 62 | AACGAGGGAUGCUCUACUA | UAGUAGAGCAUCCCUCGUU | | [479-497] |
| 63 | CGAGAGAGUCUUCCAGGUA | UACCUGGAAGACUCUCUCG | | [2095-2113] |
| 64 | CAAGUGGAGGCACUCUUCA | UGAAGAGUGCCUCCACUUG | | [1633-1651] |
| 65 | AGCUGUUUCGACCAAAUGA | UCAUUUGGUCGAAACAGCU | | [794-812] |
| 66 | GAGAAGGCCUUUACCAGAA | UUCUGGUAAAGGCCUUCUC | | [421-439] |
| 67 | GGAACAUUGUCUUUGUCUU | AAGACAAAGACAAUGUUCC | | [1043-1061] |
| 68 | AAGAGCCUAAGGAAGUGAA | UUCACUUCCUUAGGCUCUU | Rat | [1268-1286] |
| 69 | CACAAUAAAAAAAAACCUA | UAGGUUUUUUUUUAUUGUG | | [2212-2230] |
| 70 | AGAUCUUCAGGGCUCUGGA | UCCAGAGCCCUGAAGAUCU | Rat | [956-974] |
| 71 | CUAAUAACCAGACAACAGA | UCUGUUGUCUGGUUAUUAG | | [1592-1610] |
| 72 | UGCUAAUAACCAGACAACA | UGUUGUCUGGUUAUUAGCA | | [1590-1608] |
| 73 | UGGAAGUGUUAACUAUUUA | UAAAUAGUUAACACUUCCA | | [1934-1952] |
| 74 | AGAACAUGACUGAAGCAGA | UCUGCUUCAGUCAUGUUCU | | [404-422] |
| 75 | GCUCUUAAGGCUUGAAGUA | UACUUCAAGCCUUAAGAGC | | [2314-2332] |
| 76 | GAAGAACAUGACUGAAGCA | UGCUUCAGUCAUGUUCUUC | | [402-420] |
| 77 | GGCAAGCUGUUUCGACCAA | UUGGUCGAAACAGCUUGCC | | [790-808] |
| 78 | CUGCAACUACCUUGAACCA | UGGUUCAAGGUAGUUGCAG | GP | [1122-1140] |
| 79 | UACUCAUAAGGCAUAUACA | UGUAUAUGCCUUAUGAGUA | | [2344-2362] |
| 80 | GGAAGUGAAGCUCAGUGUU | AACACUGAGCUUCACUUCC | | [1278-1296] |
| 81 | AUUCCCUGUUAAAAUUUAA | UUAAAUUUUAACAGGGAAU | | [1952-1970] |
| 82 | GUUUGGAAGUGUUAACUAU | AUAGUUAACACUUCCAAAC | | [1931-1949] |
| 83 | UGGGUUUGUGCCUGAGACA | UGUCUCAGGCACAAACCCA | | [999-1017] |
| 84 | CUUUCAGAAGACAGCAUGA | UCAUGCUGUCUUCUGAAAG | | [1465-1483] |
| 85 | GUUAAAAUUUAACCUACUA | UAGUAGGUUAAAUUUUAAC | | [1959-1977] |
| 86 | AAGAAGGAGGAAUGGAAAA | UUUUCCAUUCCUCCUUCUU | GP | [649-667] |
| 87 | UAACAUUGCUUUCAUGUAU | AUACAUGAAAGCAAUGUUA | | [627-645] |
| 88 | AAAAAAACCUAGCAUCUUA | UAAGAUGCUAGGUUUUUUU | | [2220-2238] |
| 89 | UGCCUGAGACAAAAGAAGA | UCUUCUUUUGUCUCAGGCA | | [1007-1025] |
| 90 | ACUAGACAAUGAUGUGAGU | ACUCACAUCAUUGUCUAGU | | [1974-1992] |
| 91 | CAAGAAGGACUGGAAGGGA | UCCCUUCCAGUCCUUCUUG | | [306-324] |
| 92 | UUAUGAGGCUACCCAACCA | UGGUUGGGUAGCCUCAUAA | | [1653-1671] |
| 93 | UUGUCUUGAAGAAGGGCAA | UUGCCCUUCUUCAAGACAA | | [1055-1073] |
| 94 | GUUUCGACCAAAUGAGAGA | UCUCUCAUUUGGUCGAAAC | | [798-816] |
| 95 | UGUGGUGGAUCAAGACAGU | ACUGUCUUGAUCCACCACA | | [873-891] |
| 96 | CUGAGCCCAGACAUUCCAA | UUGGAAUGUCUGGGCUCAG | | [707-725] |
| 97 | AUAGGUUACUCAUAAGGCA | UGCCUUAUGAGUAACCUAU | | [2338-2356] |
| 98 | CCACAAUAAAAAAAAACCU | AGGUUUUUUUUUAUUGUGG | | [2211-2229] |
| 99 | CAAAUAUUCCUGUUUCUGA | UCAGAAACAGGAAUAUUUG | | [2279-2297] |
| 100 | AAGACUGCGCAACUACAGA | UCUGUAGUUGCGCAGUCUU | | [1787-1805] |
| 101 | AAGAAGAGCUCCAGGUCAU | AUGACCUGGAGCUCUUCUU | | [1019-1037] |
| 102 | GAAGUGUCUGGAAAGGGCA | UGCCCUUUCCAGACACUUC | | [2076-2094] |
| 103 | ACUAUUUAUUCCCUGUUAA | UUAACAGGGAAUAAAUAGU | | [1945-1963] |
| 104 | GAAGAAGGGCAAUGAUAAC | GUUAUCAUUGCCCUUCUUC | | [1062-1080] |
| 105 | GGAGAGAAGUCUAGGAUGU | ACAUCCUAGACUUCUCUCC | | [1820-1838] |
| 106 | UAGCAUCUUACAAAAACAA | UUGUUUUUGUAAGAUGCUA | | [2229-2247] |
| 107 | AAGGAGUAAGUACAAAUAU | AUAUUUGUACUUACUCCUU | | [2267-2285] |
| 108 | UGGAGGCACUCUUCAGUUA | UAACUGAAGAGUGCCUCCA | | [1637-1655] |
| 109 | AAAGGUGAAUGAAGAAUGG | CCAUUCUUCAUUCACCUUU | | [1716-1734] |
| 110 | AACCCAAGGAAAGUGAAAA | UUUUCACUUUCCUUGGGUU | GP | [1565-1583] |
| 111 | GCCUUUACCAGAAGCAUUA | UAAUGCUUCUGGUAAAGGC | | [427-445] |
| 112 | GAAGGAUUACCUAGGCAAG | CUUGCCUAGGUAAUCCUUC | | [837-855] |
| 113 | UCAUAAGGCAUAUACAAAU | AUUUGUAUAUGCCUUAUGA | | [2347-2365] |
| 114 | AAGUCUAGGAUGUUUCACA | UGUGAAACAUCCUAGACUU | | [1826-1844] |
| 115 | ACCAGACAACAGAACCUCA | UGAGGUUCUGUUGUCUGGU | | [1598-1616] |
| 116 | GAGGCACUCUUCAGUUAUG | CAUAACUGAAGAGUGCCUC | | [1639-1657] |
| 117 | UAAAAUUUAACCUACUAGA | UCUAGUAGGUUAAAUUUUA | | [1961-1979] |
| 118 | CAGCUCCUCCUAGUUCCAA | UUGGAACUAGGAGGAGCUG | | [1205-1223] |
| 119 | GAUGCUCUACUACCAGACA | UGUCUGGUAGUAGAGCAUC | | [486-504] |
| 120 | UCAUACCACAAUAAAAAAA | UUUUUUUAUUGUGGUAUGA | | [2206-2224] |
| 121 | GCAGAGAAGGCCUUUACCA | UGGUAAAGGCCUUCUCUGC | | [418-436] |
| 122 | AUAACAUUGCUUUCAUGUA | UACAUGAAAGCAAUGUUAU | | [626-644] |
| 123 | GAUUUGGAAAGCACUCGGA | UCCGAGUGCUUUCCAAAUC | | [1804-1822] |
| 124 | AGGAAAAGUGGAUGGAAGU | ACUUCCAUCCACUUUUCCU | | [2062-2080] |
| 125 | AAGGUGCACUACAAGUACA | UGUACUUGUAGUGCACCUU | GP | [1315-1333] |
| 126 | CAGAACCUCAGCUUAAGAA | UUCUUAAGCUGAGGUUCUG | | [1607-1625] |
| 127 | AAGCUGAUGCUAAUAACCA | UGGUUAUUAGCAUCAGCUU | | [1583-1601] |
| 128 | GAAGACUGCGCAACUACAG | CUGUAGUUGCGCAGUCUUC | | [1786-1804] |
| 129 | GAAGCAGAAGCUAUAUGAG | CUCAUAUAGCUUCUGCUUC | | [753-771] |
| 130 | AGAGAAGGCCUUUACCAGA | UCUGGUAAAGGCCUUCUCU | | [420-438] |
| 131 | UGAGGUGUUAUAUAACAUU | AAUGUUAUAUAACACCUCA | | [615-633] |
| 132 | AUGUUUCACAAACUACAAA | UUUGUAGUUUGUGAAACAU | | [1835-1853] |
| 133 | GUUAUCAAAGGUGAAUGAA | UUCAUUCACCUUUGAUAAC | | [1710-1728] |
| 134 | ACUGUACUGAGACAUUACA | UGUAAUGUCUCAGUACAGU | | [1912-1930] |
| 135 | UAUAUAACAUUGCUUUCAU | AUGAAAGCAAUGUUAUAUA | | [623-641] |
| 136 | AUGAGGCUACCCAACCAGA | UCUGGUUGGGUAGCCUCAU | | [1655-1673] |
| 137 | UUUCCUGAUGCAUUUGCAA | UUGCAAAUGCAUCAGGAAA | | [70-88] |
| 138 | GAUGGAAGUGUCUGGAAAG | CUUUCCAGACACUUCCAUC | | [2072-2090] |
| 139 | UACACUAUCCUGAAGAACA | UGUUCUUCAGGAUAGUGUA | | [391-409] |
| 140 | AUACUGUACUGAGACAUUA | UAAUGUCUCAGUACAGUAU | | [1910-1928] |
| 141 | CCUAAGGAAGUGAAGCUCA | UGAGCUUCACUUCCUUAGG | | [1273-1291] |
| 142 | CAAUGAUGUGAGUACCCAG | CUGGGUACUCACAUCAUUG | | [1980-1998] |
| 143 | GGUGAAUGGAGGAGUUAGG | CCUAACUCCUCCAUUCACC | | [2039-2057] |
| 144 | AGAGCCUAAGGAAGUGAAG | CUUCACUUCCUUAGGCUCU | Rat | [1269-1287] |
| 145 | ACUCUGUGGUGUGAGAACA | UGUUCUCACACCACAGAGU | | [1519-1537] |
| 146 | ACAGGUGAAUGGAGGAGUU | AACUCCUCCAUUCACCUGU | | [2036-2054] |
| 147 | GCUAAUAACCAGACAACAG | CUGUUGUCUGGUUAUUAGC | | [1591-1609] |
| 148 | AUAAGGCAUAUACAAAUAA | UUAUUUGUAUAUGCCUUAU | | [2349-2367] |
| 149 | UCGAGGGAACCAGCUGAUA | UAUCAGCUGGUUCCCUCGA | | [555-573] |
| 150 | GAGAGAAGUCUAGGAUGUU | AACAUCCUAGACUUCUCUC | | [1821-1839] |
| 151 | AAAGCUGAAGAAAAUGAAG | CUUCAUUUUCUUCAGCUUU | | [1851-1869] |
| 152 | AAGGAGGAAUGGAAAAAAG | CUUUUUUCCAUUCCUCCUU | | [652-670] |
| 153 | UUGAAGAAGGGCAAUGAUA | UAUCAUUGCCCUUCUUCAA | | [1060-1078] |
| 154 | GCUUGUUCCCUGCAACUAC | GUAGUUGCAGGGAACAAGC | GP | [1113-1131] |
| 155 | ACUACCAGACAGAGAAAUA | UAUUUCUCUGUCUGGUAGU | | [494-512] |
| 156 | UAAUCAUGUCCCUGGUGGA | UCCACCAGGGACAUGAUUA | | [248-266] ORF |
| 157 | GACCUUAAAGAAGCCUUGA | UCAAGGCUUCUUUAAGGUC | | [529-547] |
| 158 | CACCGUGUGCUAUUUGGGU | ACCCAAAUAGCACACGGUG | | [985-1003] |
| 159 | ACAUGGUGUCUAAGAAACU | AGUUUCUUAGACACCAUGU | | [1382-1400] |
| 160 | AGAUGAACCCAAGGAAAGU | ACUUUCCUUGGGUUCAUCU | | [1560-1578] |
| 161 | CUCUGUGGUGUGAGAACAC | GUGUUCUCACACCACAGAG | | [1520-1538] |
| 162 | AAGCAUUAACCGAGACAAG | CUUGUCUCGGUUAAUGCUU | | [438-456] |
| 163 | CAAGGUGCACUACAAGUAC | GUACUUGUAGUGCACCUUG | GP | [1314-1332] |
| 164 | GACUCUGUGGUGUGAGAAC | GUUCUCACACCACAGAGUC | | [1518-1536] |
| 165 | AAAUCGACAAGGCGAUGGA | UCCAUCGCCUUGUCGAUUU | | [725-743] |
| 166 | GUCUGGAAGCAGAAGCUAU | AUAGCUUCUGCUUCCAGAC | | [748-766] |
| 167 | AACUACAAAGCUGAAGAAA | UUUCUUCAGCUUUGUAGUU | | [1845-1863] |
| 168 | AAAUGAAGCCCUAUUACUU | AAGUAAUAGGGCUUCAUUU | | [1862-1880] |
| 169 | CAGAUGAACCCAAGGAAAG | CUUUCCUUGGGUUCAUCUG | | [1559-1577] |
| 170 | GUGGAUCAAGACAGUUUCU | AGAAACUGUCUUGAUCCAC | | [877-895] |
| 171 | AUGUGAGUACCCAGGAUGA | UCAUCCUGGGUACUCACAU | | [1985-2003] |
| 172 | GAACCAUUACUGUAAUUGG | CCAAUUACAGUAAUGGUUC | | [2296-2314] |
| 173 | GCUUACUUCCAACGAGGGA | UCCCUCGUUGGAAGUAAGC | GP | [469-487] |
| 174 | GCACCCUUCUGCUGUAUAC | GUAUACAGCAGAAGGGUGC | | [1895-1913] |
| 175 | ACUACAGAUUUGGAAAGCA | UGCUUUCCAAAUCUGUAGU | | [1798-1816] |
| 176 | AGUUUGGAAGUGUUAACUA | UAGUUAACACUUCCAAACU | | [1930-1948] |
| 177 | AAAUUUAACCUACUAGACA | UGUCUAGUAGGUUAAAUUU | | [1963-1981] |
| 178 | UGUUGAAGACUGCGCAACU | AGUUGCGCAGUCUUCAACA | | [1782-1800] |
| 179 | UGUAUACUGUACUGAGACA | UGUCUCAGUACAGUAUACA | | [1907-1925] |
| 180 | CAAAGGUGAAUGAAGAAUG | CAUUCUUCAUUCACCUUUG | | [1715-1733] |
| 181 | ACAGAACCUCAGCUUAAGA | UCUUAAGCUGAGGUUCUGU | | [1606-1624] |
| 182 | CAUGUACACUAUCCUGAAG | CUUCAGGAUAGUGUACAUG | | [387-405] |
| 183 | GUUUCUGAACCAUUACUGU | ACAGUAAUGGUUCAGAAAC | | [2290-2308] |
| 184 | AAAGAAGAGCUCCAGGUCA | UGACCUGGAGCUCUUCUUU | | [1018-1036] |
| 185 | UUUGUUGAAGACUGCGCAA | UUGCGCAGUCUUCAACAAA | | [1780-1798] |
| 186 | UCCAACGAGGGAUGCUCUA | UAGAGCAUCCCUCGUUGGA | GP | [476-494] |
| 187 | GGAUUUGCUUCAACAUUGG | CCAAUGUUGAAGCAAAUCC | | [365-383] |
| 188 | UACAGUUUGGAAGUGUUAA | UUAACACUUCCAAACUGUA | | [1927-1945] |
| 189 | CCUGUUAAAAUUUAACCUA | UAGGUUAAAUUUUAACAGG | | [1956-1974] |
| 190 | AAAAGCUGAUGCUAAUAAC | GUUAUUAGCAUCAGCUUUU | | [1581-1599] |
| 191 | AAACAAAAAGAAGAGCCUA | UAGGCUCUUCUUUUUGUUU | | [1258-1276] |
| 192 | CAGGCCCAAGGAGUAAGUA | UACUUACUCCUUGGGCCUG | | [2260-2278] |
| 193 | AAGUCUGAGCCCAGACAUU | AAUGUCUGGGCUCAGACUU | | [703-721] |
| 194 | GAAAGCACUCGGAGAGAAG | CUUCUCUCCGAGUGCUUUC | | [1810-1828] |
| 195 | UAUUUAUUCCCUGUUAAAA | UUUUAACAGGGAAUAAAUA | | [1947-1965] |
| 196 | AAAACCUAGCAUCUUACAA | UUGUAAGAUGCUAGGUUUU | | [2223-2241] |
| 197 | GUGGAUGGAAGUGUCUGGA | UCCAGACACUUCCAUCCAC | | [2069-2087] |
| 198 | AAAAAGAAGAGCCUAAGGA | UCCUUAGGCUCUUCUUUUU | Rat | [1262-1280] |
| 199 | CAUUGUCUUUGUCUUGAAG | CUUCAAGACAAAGACAAUG | | [1047-1065] |
| 200 | AUUUCCUGAUGCAUUUGCA | UGCAAAUGCAUCAGGAAAU | | [69-87] |
| 201 | CUAUUACUUGUUUGUAAGA | UCUUACAAACAAGUAAUAG | | [1872-1890] |
| 202 | UGCUGUAUACUGUACUGAG | CUCAGUACAGUAUACAGCA | | [1904-1922] |
| 203 | UGUUAAAAUUUAACCUACU | AGUAGGUUAAAUUUUAACA | | [1958-1976] |
| 204 | CAGACAUUCCAAAAUCGAC | GUCGAUUUUGGAAUGUCUG | | [714-732] |
| 205 | AGUGAAAAAGCUGAUGCUA | UAGCAUCAGCUUUUUCACU | | [1576-1594] |
| 206 | GAUUCAGCUUCGAGGGAAC | GUUCCCUCGAAGCUGAAUC | | [546-564] |
| 207 | GGCUUUCCAGAUGAACCCA | UGGGUUCAUCUGGAAAGCC | | [1552-1570] |
| 208 | UGGAAAGCACUCGGAGAGA | UCUCUCCGAGUGCUUUCCA | | [1808-1826] |
| 209 | UCUUGAAGAAGGGCAAUGA | UCAUUGCCCUUCUUCAAGA | | [1058-1076] |
| 210 | CAAAAACAAGACACCCAAG | CUUGGGUGUCUUGUUUUUG | | [2239-2257] |
| 211 | UUUGGAAAGCACUCGGAGA | UCUCCGAGUGCUUUCCAAA | | [1806-1824] |
| 212 | AUGGUAGGGUUAUGAGUCA | UGACUCAUAACCCUACCAU | | [31-49] |
| 213 | CGGAUUUGCUUCAACAUUG | CAAUGUUGAAGCAAAUCCG | | [364-382] |
| 214 | UUCCCUGCAACUACCUUGA | UCAAGGUAGUUGCAGGGAA | GP | [1118-1136] |
| 215 | CCUACUAGACAAUGAUGUG | CACAUCAUUGUCUAGUAGG | | [1971-1989] |
| 216 | AGAAGACAGCAUGAAGGAU | AUCCUUCAUGCUGUCUUCU | | [1470-1488] |
| 217 | UUAACCUACUAGACAAUGA | UCAUUGUCUAGUAGGUUAA | | [1967-1985] |
| 218 | AAAAAGCUGAUGCUAAUAA | UUAUUAGCAUCAGCUUUUU | | [1580-1598] |
| 219 | CUUUCCAGAUGAACCCAAG | CUUGGGUUCAUCUGGAAAG | | [1554-1572] |
| 220 | AGUGGAGGCACUCUUCAGU | ACUGAAGAGUGCCUCCACU | | [1635-1653] |
| 221 | CUGUGAGGUGUUAUAUAAC | GUUAUAUAACACCUCACAG | | [612-630] |
| 222 | CAUGACUGAAGCAGAGAAG | CUUCUCUGCUUCAGUCAUG | | [408-426] |
| 223 | GGUGAAAAACUACUGCCUG | CAGGCAGUAGUUUUUCACC | | [1500-1518] |
| 224 | UUCACACUGUAAACAUUCA | UGAAUGUUUACAGUGUGAA | | [2156-2174] |
| 225 | AAUGGAAAAAAGCUGAAGA | UCUUCAGCUUUUUUCCAUU | | [659-677] |
| 226 | UUACAGUUUGGAAGUGUUA | UAACACUUCCAAACUGUAA | | [1926-1944] |
| 227 | CUCUACUACCAGACAGAGA | UCUCUGUCUGGUAGUAGAG | | [490-508] |
| 228 | GUGACCAAGGCUUUCCAGA | UCUGGAAAGCCUUGGUCAC | | [1544-1562] |
| 229 | AGGACCUGGAGUUUCAGGA | UCCUGAAACUCCAGGUCCU | | [1673-1691] |
| 230 | GGAGUAAGUACAAAUAUUC | GAAUAUUUGUACUUACUCC | | [2269-2287] |
| 231 | AGUGGGUGACCAAGGCUUU | AAAGCCUUGGUCACCCACU | | [1539-1557] |
| 232 | AACAAGACACCCAAGUCCA | UGGACUUGGGUGUCUUGUU | | [2243-2261] |
| 233 | AGUGUUCCCAUGCCCUACA | UGUAGGGCAUGGGAACACU | | [1291-1309] |
| 234 | CAGAGAUCUUCAGGGCUCU | AGAGCCCUGAAGAUCUCUG | Rat | [953-971] |
| 235 | UUGUGCCUGAGACAAAAGA | UCUUUUGUCUCAGGCACAA | | [1004-1022] |
| 236 | GAUUUAGCACCCUUCUGCU | AGCAGAAGGGUGCUAAAUC | | [1889-1907] |
| 237 | AAGUGAAAAAGCUGAUGCU | AGCAUCAGCUUUUUCACUU | | [1575-1593] |
| 238 | AACCUUAUAGGUUACUCAU | AUGAGUAACCUAUAAGGUU | | [2332-2350] |
| 239 | UAUCCUGAAGAACAUGACU | AGUCAUGUUCUUCAGGAUA | | [396-414] |
| 240 | UACAGAUUUGGAAAGCACU | AGUGCUUUCCAAAUCUGUA | | [1800-1818] |
| 241 | UCAAGGUGCACUACAAGUA | UACUUGUAGUGCACCUUGA | GP | [1313-1331] |
| 242 | GUUUCUACCUAAUCAUGUC | GACAUGAUUAGGUAGAAAC | | [239-257] ORF |
| 243 | GUGUUAUAUAACAUUGCUU | AAGCAAUGUUAUAUAACAC | | [619-637] |
| 244 | AGAGAAGUCUAGGAUGUUU | AAACAUCCUAGACUUCUCU | | [1822-1840] |
| 245 | AACUAUUUAUUCCCUGUUA | UAACAGGGAAUAAAUAGUU | | [1944-1962] |
| 246 | UAUUCCCUGUUAAAAUUUA | UAAAUUUUAACAGGGAAUA | | [1951-1969] |
| 247 | CAAGUACACGGUAGUCAUG | CAUGACUACCGUGUACUUG | | [1326-1344] |
| 248 | CAAAAAGAAGAGCCUAAGG | CCUUAGGCUCUUCUUUUUG | Rat | [1261-1279] |
| 249 | AAAGAAGAGCCUAAGGAAG | CUUCCUUAGGCUCUUCUUU | Rat | [1264-1282] |
| 250 | UUGGAAGUGUUAACUAUUU | AAAUAGUUAACACUUCCAA | | [1933-1951] |
| 251 | AGUUAUGAGGCUACCCAAC | GUUGGGUAGCCUCAUAACU | | [1651-1669] |
| 252 | AAGUGGAGGCACUCUUCAG | CUGAAGAGUGCCUCCACUU | | [1634-1652] |
| 253 | CUGAGACAUUACAGUUUGG | CCAAACUGUAAUGUCUCAG | | [1918-1936] |
| 254 | CACCCUUCUGCUGUAUACU | AGUAUACAGCAGAAGGGUG | | [1896-1914] |
| 255 | CUGCAUGUACACUAUCCUG | CAGGAUAGUGUACAUGCAG | | [384-402] |
| 256 | AGGGAUGCUCUACUACCAG | CUGGUAGUAGAGCAUCCCU | | [483-501] |
| 257 | AAGAAGGAUUACCUAGGCA | UGCCUAGGUAAUCCUUCUU | | [835-853] |
| 258 | CACGGUAGUCAUGAAGACU | AGUCUUCAUGACUACCGUG | | [1332-1350] |
| 259 | UGUGAGUACCCAGGAUGAU | AUCAUCCUGGGUACUCACA | | [1986-2004] |
| 260 | AAUAAAAAAAAACCUAGCA | UGCUAGGUUUUUUUUUAUU | | [2215-2233] |
| 261 | GAAGUGUUAACUAUUUAUU | AAUAAAUAGUUAACACUUC | | [1936-1954] |
| 262 | GGUAGUCAUGAAGACUCAG | CUGAGUCUUCAUGACUACC | | [1335-1353] |
| 263 | UACUUGUUUGUAAGAUUUA | UAAAUCUUACAAACAAGUA | | [1876-1894] |
| 264 | AUUUAUUCCCUGUUAAAAU | AUUUUAACAGGGAAUAAAU | | [1948-1966] |
| 265 | UAUUUGGGUUUGUGCCUGA | UCAGGCACAAACCCAAAUA | | [995-1013] |
| 266 | CAGAGAAGGCCUUUACCAG | CUGGUAAAGGCCUUCUCUG | | [419-437] |
| 267 | CUGAGAAGUUAUCUUAAGG | CCUUAAGAUAACUUCUCAG | | [91-109] |
| 268 | UCAAGCUGUUUGCCUGUGA | UCACAGGCAAACAGCUUGA | Ms | [599-617] |
| 269 | AAGAAGAGCCUAAGGAAGU | ACUUCCUUAGGCUCUUCUU | Rat | [1265-1283] |
| 270 | CACGAGCAUGAAGUCUGAG | CUCAGACUUCAUGCUCGUG | | [693-711] |
| 271 | ACACUCAAGGUGCACUACA | UGUAGUGCACCUUGAGUGU | GP | [1309-1327] |
| 272 | UAACCUUAUAGGUUACUCA | UGAGUAACCUAUAAGGUUA | | [2331-2349] |
| 273 | UGUCUUGAAGAAGGGCAAU | AUUGCCCUUCUUCAAGACA | | [1056-1074] |
| 274 | AGGGAAGGUGGGCAUUUUC | GAAAAUGCCCACCUUCCCU | | [1752-1770] |
| 275 | CAAAGGGAAGGUGGGCAUU | AAUGCCCACCUUCCCUUUG | | [1749-1767] |
| 276 | CAUGGUGUCUAAGAAACUG | CAGUUUCUUAGACACCAUG | | [1383-1401] |
| 277 | ACAUUUGGUGCACUUUUCU | AGAAAAGUGCACCAAAUGU | | [2184-2202] |
| 278 | AAAAACCUAGCAUCUUACA | UGUAAGAUGCUAGGUUUUU | | [2222-2240] |
| 279 | AAGACAGCAUGAAGGAUGC | GCAUCCUUCAUGCUGUCUU | | [1472-1490] |
| 280 | UUUCAGAAGACAGCAUGAA | UUCAUGCUGUCUUCUGAAA | | [1466-1484] |
| 281 | GUGAGUACCCAGGAUGAUU | AAUCAUCCUGGGUACUCAC | | [1987-2005] |
| 282 | AAAGCUGAAGAACAGUUAG | CUAACUGUUCUUCAGCUUU | | [667-685] |
| 283 | AGAGGACCUGGAGUUUCAG | CUGAAACUCCAGGUCCUCU | | [1671-1689] |
| 284 | GGGUUAUGAGUCAGUUGCC | GGCAACUGACUCAUAACCC | | [37-55] |
| 285 | CAUGUUCAACGGGCAGAAG | CUUCUGCCCGUUGAACAUG | | [1092-1110] |
| 286 | UCAUCAAGCUGUACAUUUG | CAAAUGUACAGCUUGAUGA | | [2172-2190] |
| 287 | AAAAUGAAGCCCUAUUACU | AGUAAUAGGGCUUCAUUUU | | [1861-1879] |
| 288 | UGUGGGCAAGCUGUUUCGA | UCGAAACAGCUUGCCCACA | | [786-804] |
| 289 | UACAAAUAUUCCUGUUUCU | AGAAACAGGAAUAUUUGUA | | [2277-2295] |
| 290 | GGUGUCUAAGAAACUGGAG | CUCCAGUUUCUUAGACACC | | [1386-1404] |
| 291 | UACACGGUAGUCAUGAAGA | UCUUCAUGACUACCGUGUA | | [1330-1348] |
| 292 | UAUUCCUGUUUCUGAACCA | UGGUUCAGAAACAGGAAUA | | [2283-2301] |
| 293 | GAUUACCUAGGCAAGGCGA | UCGCCUUGCCUAGGUAAUC | | [841-859] |
| 294 | CCUUGAUUCAGCUUCGAGG | CCUCGAAGCUGAAUCAAGG | | [542-560] |
| 295 | UAUACUGUACUGAGACAUU | AAUGUCUCAGUACAGUAUA | | [1909-1927] |
| 296 | UUAAAGAAGCCUUGAUUCA | UGAAUCAAGGCUUCUUUAA | | [533-551] |
| 297 | ACAACAGAACCUCAGCUUA | UAAGCUGAGGUUCUGUUGU | | [1603-1621] |
| 298 | ACUCAAGGUGCACUACAAG | CUUGUAGUGCACCUUGAGU | GP | [1311-1329] |
| 299 | CAUCAAGCUGUACAUUUGG | CCAAAUGUACAGCUUGAUG | | [2173-2191] |
| 300 | UAACCAGACAACAGAACCU | AGGUUCUGUUGUCUGGUUA | | [1596-1614] |
| 301 | CUUGUUCCCUGCAACUACC | GGUAGUUGCAGGGAACAAG | GP | [1114-1132] |
| 302 | AAAACUACUGCCUGACUCU | AGAGUCAGGCAGUAGUUUU | Ms | [1505-1523] |
| 303 | CUGUACUGAGACAUUACAG | CUGUAAUGUCUCAGUACAG | | [1913-1931] |
| 304 | GAUCAAGACAGUUUCUCUG | CAGAGAAACUGUCUUGAUC | | [880-898] |
| 305 | GAAGGGCAAUGAUAACUGG | CCAGUUAUCAUUGCCCUUC | | [1065-1083] |
| 306 | UCAUGGUAGGGUUAUGAGU | ACUCAUAACCCUACCAUGA | | [29-47] |
| 307 | UGAAGAACAUGACUGAAGC | GCUUCAGUCAUGUUCUUCA | | [401-419] |
| 308 | UUCGAGGGAACCAGCUGAU | AUCAGCUGGUUCCCUCGAA | | [554-572] |
| 309 | AAGAAGGACUGGAAGGGAG | CUCCCUUCCAGUCCUUCUU | | [307-325] |
| 310 | AAGUACAAAUAUUCCUGUU | AACAGGAAUAUUUGUACUU | | [2274-2292] |
| 311 | UUUGGAAGUGUUAACUAUU | AAUAGUUAACACUUCCAAA | | [1932-1950] |
| 312 | AAAGGGCACGAGAGAGUCU | AGACUCUCUCGUGCCCUUU | | [2087-2105] |
| 313 | UAAGGAAGUGAAGCUCAGU | ACUGAGCUUCACUUCCUUA | | [1275-1293] |
| 314 | GAACAUUGUCUUUGUCUUG | CAAGACAAAGACAAUGUUC | | [1044-1062] |
| 315 | CACUCGGAGAGAAGUCUAG | CUAGACUUCUCUCCGAGUG | | [1815-1833] |
| 316 | CAAAGACCUUAAAGAAGCC | GGCUUCUUUAAGGUCUUUG | | [525-543] |
| 317 | AGUUUCUACCUAAUCAUGU | ACAUGAUUAGGUAGAAACU | | [238-256] ORF |
| 318 | CUCAAGGUGCACUACAAGU | ACUUGUAGUGCACCUUGAG | GP | [1312-1330] |
| 319 | AAAGCACUCGGAGAGAAGU | ACUUCUCUCCGAGUGCUUU | | [1811-1829] |
| 320 | CAAUAAAAAAAAACCUAGC | GCUAGGUUUUUUUUUAUUG | | [2214-2232] |
| 321 | AAAAGUGGAUGGAAGUGUC | GACACUUCCAUCCACUUUU | | [2065-2083] |
| 322 | AUAUAACAUUGCUUUCAUG | CAUGAAAGCAAUGUUAUAU | | [624-642] |
| 323 | UGUUAUAUAACAUUGCUUU | AAAGCAAUGUUAUAUAACA | | [620-638] |
| 324 | AAGUACACGGUAGUCAUGA | UCAUGACUACCGUGUACUU | | [1327-1345] |
| 325 | AUUUAACCUACUAGACAAU | AUUGUCUAGUAGGUUAAAU | | [1965-1983] |
| 326 | AGGGAACCAGCUGAUAGAC | GUCUAUCAGCUGGUUCCCU | | [558-576] |
| 327 | AGGUGAAUGGAGGAGUUAG | CUAACUCCUCCAUUCACCU | | [2038-2056] |
| 328 | UUAUCAAAGGUGAAUGAAG | CUUCAUUCACCUUUGAUAA | | [1711-1729] |
| 329 | CACUCCCGGAUUUGCUUCA | UGAAGCAAAUCCGGGAGUG | | [358-376] |
| 330 | UUUUGUUGAAGACUGCGCA | UGCGCAGUCUUCAACAAAA | | [1779-1797] |
| 331 | UACUGUACUGAGACAUUAC | GUAAUGUCUCAGUACAGUA | | [1911-1929] |
| 332 | UGUCUGGAAGCAGAAGCUA | UAGCUUCUGCUUCCAGACA | | [747-765] |
| 333 | UAGGUUACUCAUAAGGCAU | AUGCCUUAUGAGUAACCUA | | [2339-2357] |
| 334 | AAAAAAAAACCUAGCAUCU | AGAUGCUAGGUUUUUUUUU | | [2218-2236] |
| 335 | GAGUAAGUACAAAUAUUCC | GGAAUAUUUGUACUUACUC | | [2270-2288] |
| 336 | CAUUAACCGAGACAAGCAC | GUGCUUGUCUCGGUUAAUG | | [441-459] |
| 337 | ACAAGACACCCAAGUCCAG | CUGGACUUGGGUGUCUUGU | | [2244-2262] |
| 338 | CAUUUGCAACACUGAGAAG | CUUCUCAGUGUUGCAAAUG | | [80-98] |
| 339 | AUUACAGUUUGGAAGUGUU | AACACUUCCAAACUGUAAU | | [1925-1943] |
| 340 | AGUUUUUGUUGAAGACUGC | GCAGUCUUCAACAAAAACU | | [1776-1794] |
| 341 | AAGGGCACGAGAGAGUCUU | AAGACUCUCUCGUGCCCUU | | [2088-2106] |
| 342 | AUAAUCCUGGUGUUAUCAA | UUGAUAACACCAGGAUUAU | | [1699-1717] |
| 343 | AUUACUUGUUUGUAAGAUU | AAUCUUACAAACAAGUAAU | | [1874-1892] |
| 344 | UCUUAAGGCUUGAAGUAAC | GUUACUUCAAGCCUUAAGA | | [2316-2334] |
| 345 | AUAAAAAAAAACCUAGCAU | AUGCUAGGUUUUUUUUUAU | | [2216-2234] |
| 346 | AGAAGAGCUCCAGGUCAUG | CAUGACCUGGAGCUCUUCU | | [1020-1038] |
| 347 | GAAGAACAGUUAGCAUUGG | CCAAUGCUAACUGUUCUUC | | [673-691] |
| 348 | UCUGAGCCCAGACAUUCCA | UGGAAUGUCUGGGCUCAGA | | [706-724] |
| 349 | AGAGUCUUCCAGGUACUGA | UCAGUACCUGGAAGACUCU | | [2099-2117] |
| 350 | UGAGUACCCAGGAUGAUUU | AAAUCAUCCUGGGUACUCA | | [1988-2006] |
| 351 | UCCAAAAUCGACAAGGCGA | UCGCCUUGUCGAUUUUGGA | | [721-739] |
| 352 | UGAAGAACAGUUAGCAUUG | CAAUGCUAACUGUUCUUCA | | [672-690] |
| 353 | CAGACAACAGAACCUCAGC | GCUGAGGUUCUGUUGUCUG | | [1600-1618] |
| 354 | AUGUACACUAUCCUGAAGA | UCUUCAGGAUAGUGUACAU | | [388-406] |
| 355 | UUUGCAACACUGAGAAGUU | AACUUCUCAGUGUUGCAAA | | [82-100] |
| 356 | AUCCCAGCUCCUCCUAGUU | AACUAGGAGGAGCUGGGAU | | [1201-1219] |
| 357 | UGUACUGAGACAUUACAGU | ACUGUAAUGUCUCAGUACA | | [1914-1932] |
| 358 | AACCAGUUGAGCUGCGGAU | AUCCGCAGCUCAACUGGUU | | [1136-1154] |
| 359 | CUAAGAAACUGGAGCUCCG | CGGAGCUCCAGUUUCUUAG | | [1391-1409] |
| 360 | ACUUGUUUGUAAGAUUUAG | CUAAAUCUUACAAACAAGU | | [1877-1895] |
| 361 | CUGUGGUGGAUCAAGACAG | CUGUCUUGAUCCACCACAG | GP | [872-890] |
| 362 | GAAGCCCUAUUACUUGUUU | AAACAAGUAAUAGGGCUUC | | [1866-1884] |
| 363 | GACAUUACAGUUUGGAAGU | ACUUCCAAACUGUAAUGUC | | [1922-1940] |
| 364 | GAACCUCAGCUUAAGAAAG | CUUUCUUAAGCUGAGGUUC | | [1609-1627] |
| 365 | GAUCCUGUUUCUUGCUCUG | CAGAGCAAGAAACAGGAUC | | [2116-2134] |
| 366 | CACUAUCCUGAAGAACAUG | CAUGUUCUUCAGGAUAGUG | | [393-411] |
| 367 | AUGCUAAUAACCAGACAAC | GUUGUCUGGUUAUUAGCAU | | [1589-1607] |
| 368 | UACAAAAACAAGACACCCA | UGGGUGUCUUGUUUUUGUA | | [2237-2255] |
| 369 | AAAGUGGAUGGAAGUGUCU | AGACACUUCCAUCCACUUU | | [2066-2084] |
| 370 | CAUCUUACAAAAACAAGAC | GUCUUGUUUUUGUAAGAUG | | [2232-2250] |
| 371 | GUGGUGGAUCAAGACAGUU | AACUGUCUUGAUCCACCAC | | [874-892] |
| 372 | AGCAUGAAGUCUGAGCCCA | UGGGCUCAGACUUCAUGCU | | [697-715] |
| 373 | ACUACUGCCUGACUCUGUG | CACAGAGUCAGGCAGUAGU | Ms | [1508-1526] |
| 374 | AACCAGAGGACCUGGAGUU | AACUCCAGGUCCUCUGGUU | | [1667-1685] |
| 375 | GCUCUACUACCAGACAGAG | CUCUGUCUGGUAGUAGAGC | | [489-507] |
| 376 | GAUAUAAUCCUGGUGUUAU | AUAACACCAGGAUUAUAUC | | [1696-1714] |
| 377 | AGGAGGAAUGGAAAAAAGC | GCUUUUUUCCAUUCCUCCU | | [653-671] |
| 378 | AAAGCUGAUGCUAAUAACC | GGUUAUUAGCAUCAGCUUU | | [1582-1600] |
| 379 | AGGAUGUUUCACAAACUAC | GUAGUUUGUGAAACAUCCU | | [1832-1850] |
| 380 | ACCAGAGGACCUGGAGUUU | AAACUCCAGGUCCUCUGGU | | [1668-1686] |
| 381 | AACAUUGCUUUCAUGUAUG | CAUACAUGAAAGCAAUGUU | | [628-646] |
| 382 | UUUCUGAACCAUUACUGUA | UACAGUAAUGGUUCAGAAA | | [2291-2309] |
| 383 | ACUGCGCAACUACAGAUUU | AAAUCUGUAGUUGCGCAGU | | [1790-1808] |
| 384 | AAGAAGCCUUGAUUCAGCU | AGCUGAAUCAAGGCUUCUU | | [536-554] |
| 385 | ACUAUCCUGAAGAACAUGA | UCAUGUUCUUCAGGAUAGU | | [394-412] |
| 386 | UCUACCUAAUCAUGUCCCU | AGGGACAUGAUUAGGUAGA | | [242-260] ORF |
| 387 | UAAGAUUUAGCACCCUUCU | AGAAGGGUGCUAAAUCUUA | | [1886-1904] |
| 388 | UCUCAUGGUAGGGUUAUGA | UCAUAACCCUACCAUGAGA | | [27-45] |
| 389 | GACCAAGGCUUUCCAGAUG | CAUCUGGAAAGCCUUGGUC | | [1546-1564] |
| 390 | AGCUGAAGAAAAUGAAGCC | GGCUUCAUUUUCUUCAGCU | | [1853-1871] |
| 391 | UUGUUUGUAAGAUUUAGCA | UGCUAAAUCUUACAAACAA | | [1879-1897] |
| 392 | AUCUUACAAAAACAAGACA | UGUCUUGUUUUUGUAAGAU | | [2233-2251] |
| 393 | GAUAGACUAUAAGAUCCUG | CAGGAUCUUAUAGUCUAUC | GP | [570-588] |
| 394 | AACAGAACCUCAGCUUAAG | CUUAAGCUGAGGUUCUGUU | | [1605-1623] |
| 395 | ACAAUAAAAAAAAACCUAG | CUAGGUUUUUUUUUAUUGU | | [2213-2231] |
| 396 | CUGUAUACUGUACUGAGAC | GUCUCAGUACAGUAUACAG | | [1906-1924] |
| 397 | CCUGCAACUACCUUGAACC | GGUUCAAGGUAGUUGCAGG | GP | [1121-1139] |
| 398 | CUGUUAAAAUUUAACCUAC | GUAGGUUAAAUUUUAACAG | | [1957-1975] |
| 399 | CAAGGCUUUCCAGAUGAAC | GUUCAUCUGGAAAGCCUUG | | [1549-1567] |
| 400 | GUCAUGUUCAACGGGCAGA | UCUGCCCGUUGAACAUGAC | | [1090-1108] |
| 401 | AUGAACCCAAGGAAAGUGA | UCACUUUCCUUGGGUUCAU | | [1562-1580] |
| 402 | ACUACAAGUACACGGUAGU | ACUACCGUGUACUUGUAGU | | [1322-1340] |
| 403 | AACAUGACUGAAGCAGAGA | UCUCUGCUUCAGUCAUGUU | | [406-424] |
| 404 | UUCAGAAGACAGCAUGAAG | CUUCAUGCUGUCUUCUGAA | | [1467-1485] |
| 405 | AUCUGUGGUGGAUCAAGAC | GUCUUGAUCCACCACAGAU | GP | [870-888] |
| 406 | GAGCCUAAGGAAGUGAAGC | GCUUCACUUCCUUAGGCUC | Rat | [1270-1288] |
| 407 | ACCAAAUGAGAGACAAGUG | CACUUGUCUCUCAUUUGGU | | [804-822] |
| 408 | AGUAAGUACAAAUAUUCCU | AGGAAUAUUUGUACUUACU | | [2271-2289] |
| 409 | CAACACUGAGAAGUUAUCU | AGAUAACUUCUCAGUGUUG | | [86-104] |
| 410 | AUCAGCCUCUGGAAUGAAG | CUUCAUUCCAGAGGCUGAU | | [271-289] |
| 411 | AAUCCUGGUGUUAUCAAAG | CUUUGAUAACACCAGGAUU | | [1701-1719] |
| 412 | AAGCUGUACAUUUGGUGCA | UGCACCAAAUGUACAGCUU | | [2177-2195] |
| 413 | AGUGAAGCUCAGUGUUCCC | GGGAACACUGAGCUUCACU | | [1281-1299] |
| 414 | CUUACUUCCAACGAGGGAU | AUCCCUCGUUGGAAGUAAG | GP | [470-488] |
| 415 | CACACUAAGCUGAGCUAUC | GAUAGCUCAGCUUAGUGUG | | [1417-1435] |
| 416 | AAAAAAAACCUAGCAUCUU | AAGAUGCUAGGUUUUUUUU | | [2219-2237] |
| 417 | UUUCUUGCUCUGAGUGCUA | UAGCACUCAGAGCAAGAAA | | [2123-2141] |
| 418 | GUUCAAGCUGUUUGCCUGU | ACAGGCAAACAGCUUGAAC | Ms | [597-615] |
| 419 | UAGGGUUAUGAGUCAGUUG | CAACUGACUCAUAACCCUA | | [35-53] |
| 420 | UUUGGGUUUGUGCCUGAGA | UCUCAGGCACAAACCCAAA | | [997-1015] |
| 421 | AAGUUUUUGUUGAAGACUG | CAGUCUUCAACAAAAACUU | | [1775-1793] |
| 422 | UUACUUGUUUGUAAGAUUU | AAAUCUUACAAACAAGUAA | | [1875-1893] |
| 423 | ACUACCUUGAACCAGUUGA | UCAACUGGUUCAAGGUAGU | GP | [1127-1145] |
| 424 | AGAAGUUAUCUUAAGGGAG | CUCCCUUAAGAUAACUUCU | | [94-112] |
| 425 | AUUCCUGUUUCUGAACCAU | AUGGUUCAGAAACAGGAAU | | [2284-2302] |
| 426 | AAGCUGUUUCGACCAAAUG | CAUUUGGUCGAAACAGCUU | | [793-811] |
| 427 | GUGAAGCUCAGUGUUCCCA | UGGGAACACUGAGCUUCAC | | [1282-1300] |
| 428 | AGACAGAGAAAUAUGAUUU | AAAUCAUAUUUCUCUGUCU | | [500-518] |
| 429 | CAUUUGGUGCACUUUUCUG | CAGAAAAGUGCACCAAAUG | | [2185-2203] |
| 430 | AGUUCAAGCUGUUUGCCUG | CAGGCAAACAGCUUGAACU | Ms | [596-614] |
| 431 | AAGGCCUUUACCAGAAGCA | UGCUUCUGGUAAAGGCCUU | | [424-442] |
| 432 | GUCUUUGUCUUGAAGAAGG | CCUUCUUCAAGACAAAGAC | Rat, Ms, GP | [1051-1069] |
| 433 | AAAAUUUAACCUACUAGAC | GUCUAGUAGGUUAAAUUUU | | [1962-1980] |
| 434 | UCUGUGGUGGAUCAAGACA | UGUCUUGAUCCACCACAGA | GP | [871-889] |
| 435 | UACCAGAAGCAUUAACCGA | UCGGUUAAUGCUUCUGGUA | | [432-450] |
| 436 | GAGUACCCAGGAUGAUUUC | GAAAUCAUCCUGGGUACUC | | [1989-2007] |
| 437 | UGUUCCCUGCAACUACCUU | AAGGUAGUUGCAGGGAACA | GP | [1116-1134] |
| 438 | AGACAAUGAUGUGAGUACC | GGUACUCACAUCAUUGUCU | | [1977-1995] |
| 439 | AAGCCUUGAUUCAGCUUCG | CGAAGCUGAAUCAAGGCUU | | [539-557] |
| 440 | GAAGUGAAGCUCAGUGUUC | GAACACUGAGCUUCACUUC | | [1279-1297] |
| 441 | AAUCGACAAGGCGAUGGAG | CUCCAUCGCCUUGUCGAUU | | [726-744] |
| 442 | GCUAUUUGGGUUUGUGCCU | AGGCACAAACCCAAAUAGC | | [993-1011] |
| 443 | AACAUUGUCUUUGUCUUGA | UCAAGACAAAGACAAUGUU | | [1045-1063] |
| 444 | AAAAAAGCUGAAGAACAGU | ACUGUUCUUCAGCUUUUUU | | [664-682] |
| 445 | ACUCUUCAGUUAUGAGGCU | AGCCUCAUAACUGAAGAGU | | [1644-1662] |
| 446 | CAGUUCAAGCUGUUUGCCU | AGGCAAACAGCUUGAACUG | Ms | [595-613] |
| 447 | AACUACUGCCUGACUCUGU | ACAGAGUCAGGCAGUAGUU | Ms | [1507-1525] |
| 448 | AUUGUCUUUGUCUUGAAGA | UCUUCAAGACAAAGACAAU | | [1048-1066] |
| 449 | AAAGGGAAGGUGGGCAUUU | AAAUGCCCACCUUCCCUUU | | [1750-1768] |
| 450 | CUCAUGGUAGGGUUAUGAG | CUCAUAACCCUACCAUGAG | | [28-46] |
| 451 | AUGCAUUUGCAACACUGAG | CUCAGUGUUGCAAAUGCAU | | [77-95] |
| 452 | AAGGAAAGUGAAAAAGCUG | CAGCUUUUUCACUUUCCUU | | [1570-1588] |
| 453 | CAGGUGAAAAACUACUGCC | GGCAGUAGUUUUUCACCUG | | [1498-1516] |
| 454 | AUGAUGUGAGUACCCAGGA | UCCUGGGUACUCACAUCAU | | [1982-2000] |
| 455 | AAAGAAGCCUUGAUUCAGC | GCUGAAUCAAGGCUUCUUU | | [535-553] |
| 456 | GUUUCUUGCUCUGAGUGCU | AGCACUCAGAGCAAGAAAC | | [2122-2140] |
| 457 | UAGACAAUGAUGUGAGUAC | GUACUCACAUCAUUGUCUA | | [1976-1994] |
| 458 | UGUUUCGACCAAAUGAGAG | CUCUCAUUUGGUCGAAACA | | [797-815] |
| 459 | CUUAAGGCUUGAAGUAACC | GGUUACUUCAAGCCUUAAG | | [2317-2335] |
| 460 | CAAAAUCGACAAGGCGAUG | CAUCGCCUUGUCGAUUUUG | | [723-741] |
| 461 | CUGAUAGACUAUAAGAUCC | GGAUCUUAUAGUCUAUCAG | GP | [568-586] |
| 462 | ACAGAUUUGGAAAGCACUC | GAGUGCUUUCCAAAUCUGU | | [1801-1819] |
| 463 | AAGUGUUAACUAUUUAUUC | GAAUAAAUAGUUAACACUU | | [1937-1955] |
| 464 | UUUAUUCCCUGUUAAAAUU | AAUUUUAACAGGGAAUAAA | | [1949-1967] |
| 465 | AAGUGAAGCUCAGUGUUCC | GGAACACUGAGCUUCACUU | | [1280-1298] |
| 466 | ACGAGGGAUGCUCUACUAC | GUAGUAGAGCAUCCCUCGU | | [480-498] |
| 467 | UCUGCUGUAUACUGUACUG | CAGUACAGUAUACAGCAGA | | [1902-1920] |
| 468 | AGUGGAUGGAAGUGUCUGG | CCAGACACUUCCAUCCACU | | [2068-2086] |
| 469 | AAAAACAAGACACCCAAGU | ACUUGGGUGUCUUGUUUUU | | [2240-2258] |
| 470 | UUGGGUUUGUGCCUGAGAC | GUCUCAGGCACAAACCCAA | | [998-1016] |
| 471 | CUCACCGUGUGCUAUUUGG | CCAAAUAGCACACGGUGAG | | [983-1001] |
| 472 | GUACAAAUAUUCCUGUUUC | GAAACAGGAAUAUUUGUAC | | [2276-2294] |
| 473 | CUCUCUCUGCUUCUUUCCU | AGGAAAGAAGCAGAGAGAG | | [2-20] |
| 474 | CAGAAAGUGAACACCUUGG | CCAAGGUGUUCACUUUCUG | | [140-158] |
| 475 | GACAAGAAGGACUGGAAGG | CCUUCCAGUCCUUCUUGUC | Rat, Ms | [304-322] |
| 476 | UGUUUCACAAACUACAAAG | CUUUGUAGUUUGUGAAACA | | [1836-1854] |
| 477 | AGUACAAAUAUUCCUGUUU | AAACAGGAAUAUUUGUACU | | [2275-2293] |
| 478 | AGGCUUUCCAGAUGAACCC | GGGUUCAUCUGGAAAGCCU | | [1551-1569] |
| 479 | UUCCAGAUGAACCCAAGGA | UCCUUGGGUUCAUCUGGAA | | [1556-1574] |
| 480 | UACUGAUCCUGUUUCUUGC | GCAAGAAACAGGAUCAGUA | | [2112-2130] |
| 481 | GUACACUAUCCUGAAGAAC | GUUCUUCAGGAUAGUGUAC | | [390-408] |
| 482 | GAGAGUCUUCCAGGUACUG | CAGUACCUGGAAGACUCUC | | [2098-2116] |
| 483 | UAAAGAAGCCUUGAUUCAG | CUGAAUCAAGGCUUCUUUA | | [534-552] |
| 484 | GAGACAAAAGAAGAGCUCC | GGAGCUCUUCUUUUGUCUC | | [1012-1030] |
| 485 | UGUGCUAUUUGGGUUUGUG | CACAAACCCAAAUAGCACA | | [990-1008] |
| 486 | CAUGGUAGGGUUAUGAGUC | GACUCAUAACCCUACCAUG | | [30-48] |
| 487 | UCUUUGUCUUGAAGAAGGG | CCCUUCUUCAAGACAAAGA | Rat, Ms, GP | [1052-1070] |
| 488 | ACUGAAGCAGAGAAGGCCU | AGGCCUUCUCUGCUUCAGU | | [412-430] |
| 489 | AGGCCCAAGGAGUAAGUAC | GUACUUACUCCUUGGGCCU | | [2261-2279] |
| 490 | UUAUAUAACAUUGCUUUCA | UGAAAGCAAUGUUAUAUAA | | [622-640] |
| 491 | AUCAAGCUGUACAUUUGGU | ACCAAAUGUACAGCUUGAU | | [2174-2192] |
| 492 | ACCUGUCUUCUCUCUGUCU | AGACAGAGAGAAGACAGGU | | [196-214] |
| 493 | UACCACAAUAAAAAAAAAC | GUUUUUUUUUAUUGUGGUA | | [2209-2227] |
| 494 | AGCAUCUUACAAAAACAAG | CUUGUUUUUGUAAGAUGCU | | [2230-2248] |
| 495 | UUCGACCAAAUGAGAGACA | UGUCUCUCAUUUGGUCGAA | | [800-818] |
| 496 | AUUCCAAAAUCGACAAGGC | GCCUUGUCGAUUUUGGAAU | | [719-737] |
| 497 | UAGUUUCUACCUAAUCAUG | CAUGAUUAGGUAGAAACUA | | [237-255] ORF |
| 498 | CAUUACAGUUUGGAAGUGU | ACACUUCCAAACUGUAAUG | | [1924-1942] |
| 499 | UACAUUUGGUGCACUUUUC | GAAAAGUGCACCAAAUGUA | | [2183-2201] |
| 500 | GGUGCACUACAAGUACACG | CGUGUACUUGUAGUGCACC | | [1317-1335] |

TABLE B1
P53 19-mer oligomer pairs P53 - tumor protein p53

| | | | | Human-8400737: |
|---|---|---|---|---|
| No. | Sense | AntiSense | Other Sp | 252-1433 |

| | | | | |
|---|---|---|---|---|
| 1 | GAGAAUAUUUCACCCUUCA | UGAAGGGUGAAAUAUUCUC | MK, Dog | [1221-1240] |
| 2 | CCAUUUUUAUAUCGAUCUC | GAGAUCGAUAUAAAAAUGG | | [2584-2602] |
| 3 | CCCUUUUUAUAUCCCAUUU | AAAUGGGAUAUAAAAAGGG | | [2571-2589] |
| 4 | GCCCUCAACAAGAUGUUUU | AAAACAUCUUGUUGAGGGC | | [636-654] |
| 5 | CCCAUUUUUAUAUCGAUCU | AGAUCGAUAUAAAAAUGGG | | [2583-2601] |
| 6 | CAGCUUAGAUUUUAAGGUU | AACCUUAAAAUCUAAGCUG | | [1646-1664] |
| 7 | CCAGCUUAGAUUUUAAGGU | ACCUUAAAAUCUAAGCUGG | | [1645-1663] |
| 8 | GGAAACAUUUUCAGACCUA | UAGGUCUGAAAAUGUUUCC | MK | [299-317] |
| 9 | GGAUGACAGAAACACUUUU | AAAAGUGUUUCUGUCAUCC | MK, Pig | [869-887] |
| 10 | GCCACCUGAAGUCCAAAAA | UUUUUGGACUUCAGGUGGC | | [1351-1369] |
| 11 | GGGUCAACAUCUUUUACAU | AUGUAAAAGAUGUUGACCC | | [2507-2525] |
| 12 | CCCUCAACAAGAUGUUUUG | CAAAACAUCUUGUUGAGGG | | [637-655] |
| 13 | ACAUACCAGCUUAGAUUUU | AAAAUCUAAGCUGGUAUGU | | [1640-1658] |
| 14 | UCCCAUUUUUAUAUCGAUC | GAUCGAUAUAAAAAUGGGA | | [2582-2600] |
| 15 | UCCCUUUUUAUAUCCCAUU | AAUGGGAUAUAAAAAGGGA | | [2570-2588] |
| 16 | GUGAGGGUUAAUGAAAUAA | UUAUUUCAUUAACCCUCAC | | [1865-1883] |
| 17 | CCAUAUCUGUGAAAUGCUG | CAGCAUUUCACAGAUAUGG | | [1817-1835] |
| 18 | CCCAUCCUCACCAUCAUCA | UGAUGAUGGUGAGGAUGGG | Pig, GP, RB | [999-1017] |
| 19 | CCGGACGAUAUUGAACAAU | AUUGUUCAAUAUCGUCCGG | | [390-408] |
| 20 | CCCGGACGAUAUUGAACAA | UUGUUCAAUAUCGUCCGGG | | [389-407] |
| 21 | CUCUUGUAUAUGAUGAUCU | AGAUCAUCAUAUACAAGAG | | [2131-2149] |
| 22 | CCCUCAUGUUGAAUUUUCU | AGAAAAUUCAACAUGAGGG | | [1785-1803] |
| 23 | CCACCUGAAGUCCAAAAAG | CUUUUUGGACUUCAGGUGG | | [1352-1370] |
| 24 | CCACCAAGACUUGUUUUAU | AUAAAACAAGUCUUGGUGG | | [2154-2172] |
| 25 | GCAUUGUGAGGGUUAAUGA | UCAUUAACCCUCACAAUGC | | [1860-1878] |
| 26 | CUAACUUCAAGGCCCAUAU | AUAUGGGCCUUGAAGUUAG | | [1804-1822] |
| 27 | AGUUUACAAUCAGCCACAU | AUGUGGCUGAUUGUAAACU | | [1719-1737] |
| 28 | AGGGUUAGUUUACAAUCAG | CUGAUUGUAAACUAACCCU | | [1713-1731] |
| 29 | GACUUCCAUUUGCUUUGUC | GACAAAGCAAAUGGAAGUC | | [1554-1572] |
| 30 | GAAACAUUUUCAGACCUAU | AUAGGUCUGAAAAUGUUUC | MK | [300-318] |
| 31 | GAAACACUUUUCGACAUAG | CUAUGUCGAAAAGUGUUUC | MK | [877-895] |
| 32 | UGCCCUCAACAAGAUGUUU | AAACAUCUUGUUGAGGGCA | | [635-653] |
| 33 | CGGACGAUAUUGAACAAUG | CAUUGUUCAAUAUCGUCCG | | [391-409] |
| 34 | CUCCCUUUUUAUAUCCCAU | AUGGGAUAUAAAAAGGGAG | | [2569-2587] |
| 35 | GGUCAACAUCUUUUACAUU | AAUGUAAAAGAUGUUGACC | | [2508-2526] |
| 36 | UCUUGUAUAUGAUGAUCUG | CAGAUCAUCAUAUACAAGA | | [2132-2150] |
| 37 | GCUGGUGGGUUGGUAGUUU | AAACUACCAACCCACCAGC | | [1975-1993] |
| 38 | UUGUGAGGGUUAAUGAAAU | AUUUCAUUAACCCUCACAA | | [1863-1881] |
| 39 | GGGAGAUGUAAGAAAUGUU | AACAUUUCUUACAUCUCCC | | [1684-1702] |
| 40 | CUGACAUUCUCCACUUCUU | AAGAAGUGGAGAAUGUCAG | | [1430-1448] |
| 41 | CCAAAAAGGGUCAGUCUAC | GUAGACUGACCCUUUUUGG | | [1363-1381] |
| 42 | GUAAUCUACUGGGACGGAA | UUCCGUCCCAGUAGAUUAC | MK | [1036-1054] |
| 43 | CCAUCCACUACAACUACAU | AUGUAGUUGUAGUGGAUGG | MK, Dog, GP | [943-961] |
| 44 | CCGAGUGGAAGGAAAUUUG | CAAAUUUCCUUCCACUCGG | MK, Dog | [836-854] |
| 45 | CUGUCAUCUUCUGUCCCUU | AAGGGACAGAAGAUGACAG | | [528-546] |
| 46 | CCCUCCUUCUCCCUUUUUA | UAAAAAGGGAGAAGGAGGG | | [2561-2579] |
| 47 | GGAAGGGUCAACAUCUUUU | AAAAGAUGUUGACCCUUCC | | [2503-2521] |
| 48 | CAAGACUUGUUUUAUGCUC | GAGCAUAAAACAAGUCUUG | | [2158-2176] |
| 49 | UCCACCAAGACUUGUUUUA | UAAAACAAGUCUUGGUGGA | | [2153-2171] |
| 50 | GGAGUUGUCAAGUCUUGCU | AGCAAGACUUGACAACUCC | | [2022-2040] |
| 51 | CCCAUAUCUGUGAAAUGCU | AGCAUUUCACAGAUAUGGG | | [1816-1834] |
| 52 | GGGUUAGUUUACAAUCAGC | GCUGAUUGUAAACUAACCC | | [1714-1732] |
| 53 | ACAUUCUCCACUUCUUGUU | AACAAGAAGUGGAGAAUGU | | [1433-1451] |
| 54 | CCAUCCUCACCAUCAUCAC | GUGAUGAUGGUGAGGAUGG | Pig, GP, RB | [1000-1018] |
| 55 | AAACACUUUUCGACAUAGU | ACUAUGUCGAAAAGUGUUU | MK | [878-896] |
| 56 | CCUGUCAUCUUCUGUCCCU | AGGGACAGAAGAUGACAGG | | [527-545] |
| 57 | ACGAUAUUGAACAAUGGUU | AACCAUUGUUCAAUAUCGU | | [394-412] |
| 58 | GGACGAUAUUGAACAAUGG | CCAUUGUUCAAUAUCGUCC | | [392-410] |
| 59 | ACAUCUUUUACAUUCUGCA | UGCAGAAUGUAAAAGAUGU | | [2513-2531] |
| 60 | CCAAGACUUGUUUUAUGCU | AGCAUAAAACAAGUCUUGG | | [2157-2175] |
| 61 | CACCAAGACUUGUUUUAUG | CAUAAAACAAGUCUUGGUG | | [2155-2173] |
| 62 | CGACCUUAGUACCUAAAAG | CUUUUAGGUACUAAGGUCG | | [2075-2093] |
| 63 | GGUGGGUUGGUAGUUUCUA | UAGAAACUACCAACCCACC | | [1978-1996] |
| 64 | UGCAUUGUGAGGGUUAAUG | CAUUAACCCUCACAAUGCA | | [1859-1877] |
| 65 | GUGCAUUGUGAGGGUUAAU | AUUAACCCUCACAAUGCAC | | [1858-1876] |
| 66 | GCAUUUGCACCUACCUCAC | GUGAGGUAGGUGCAAAUGC | | [1836-1854] |
| 67 | GGCCCAUAUCUGUGAAAUG | CAUUUCACAGAUAUGGGCC | | [1814-1832] |
| 68 | AGGCCCAUAUCUGUGAAAU | AUUUCACAGAUAUGGGCCU | | [1813-1831] |
| 69 | CUCAUGUUGAAUUUUCUCU | AGAGAAAAUUCAACAUGAG | | [1787-1805] |
| 70 | UCCCUCAUGUUGAAUUUUC | GAAAAUUCAACAUGAGGGA | | [1784-1802] |
| 71 | CUGUCCCUCAUGUUGAAUU | AAUUCAACAUGAGGGACAG | | [1781-1799] |
| 72 | AGCUGUCCCUCAUGUUGAA | UUCAACAUGAGGGACAGCU | | [1779-1797] |
| 73 | AGACCUAUGGAAACUACUU | AAGUAGUUUCCAUAGGUCU | MK | [311-329] |
| 74 | GUGAGCGCUUCGAGAUGUU | AACAUCUCGAAGCGCUCAC | MK, Rat | [1255-1273] |
| 75 | UGGAUGACAGAAACACUUU | AAAGUGUUUCUGUCAUCCA | Pig | [868-886] |
| 76 | GGAAGGAAAUUUGCGUGUG | CACACGCAAAUUUCCUUCC | MK | [842-860] |
| 77 | GCUAAAAGUUUUGAGCUUC | GAAGCUCAAAACUUUUAGC | | [92-110] |
| 78 | GACUGGCGCUAAAAGUUUU | AAAACUUUUAGCGCCAGUC | GP | [85-103] |
| 79 | AGGGUCAACAUCUUUUACA | UGUAAAAGAUGUUGACCCU | | [2506-2524] |
| 80 | GCCUUGAAACCACCUUUUA | UAAAAGGUGGUUUCAAGGC | | [1894-1912] |
| 81 | AGUGCAUUGUGAGGGUUAA | UUAACCCUCACAAUGCACU | | [1857-1875] |
| 82 | AGAGUGCAUUGUGAGGGUU | AACCCUCACAAUGCACUCU | | [1855-1873] |
| 83 | UGUCCCUCAUGUUGAAUUU | AAAUUCAACAUGAGGGACA | | [1782-1800] |
| 84 | GACCUAUGGAAACUACUUC | GAAGUAGUUUCCAUAGGUC | MK | [312-330] |
| 85 | GGAUGUUUGGGAGAUGUAA | UUACAUCUCCCAAACAUCC | | [1676-1694] |
| 86 | GGGAUGUUUGGGAGAUGUA | UACAUCUCCCAAACAUCCC | | [1675-1693] |
| 87 | AGGAAACAUUUUCAGACCU | AGGUCUGAAAAUGUUUCCU | MK | [298-316] |
| 88 | UGGAUGGAGAAUAUUUCAC | GUGAAAUAUUCUCCAUCCA | MK | [1219-1237] |
| 89 | AGUAUUUGGAUGACAGAAA | UUUCUGUCAUCCAAAUACU | RB | [862-880] |
| 90 | GGAGUAUUUGGAUGACAGA | UCUGUCAUCCAAAUACUCC | RB | [860-878] |
| 91 | CCUGAAAACAACGUUCUGU | ACAGAACGUUGUUUUCAGG | GP | [330-348] |
| 92 | CUGGUGGGUUGGUAGUUUC | GAAACUACCAACCCACCAG | | [1976-1994] |
| 93 | ACUUCCUGAAAACAACGUU | AACGUUGUUUUCAGGAAGU | GP | [326-344] |
| 94 | CUCAAGACUGGCGCUAAAA | UUUUAGCGCCAGUCUUGAG | | [80-98] |
| 95 | AUCUGUGAAAUGCUGGCAU | AUGCCAGCAUUUCACAGAU | | [1821-1839] |
| 96 | UCUAACUUCAAGGCCCAUA | UAUGGGCCUUGAAGUUAGA | | [1803-1821] |
| 97 | GUCCCUCAUGUUGAAUUUU | AAAAUUCAACAUGAGGGAC | | [1783-1801] |
| 98 | AGAAAUGUUCUUGCAGUUA | UAACUGCAAGAACAUUUCU | | [1694-1712] |
| 99 | CCAUUUUGGGUUUUGGGUC | GACCCAAAACCCAAAAUGG | | [1492-1510] |
| 100 | GACUCAGACUGACAUUCUC | GAGAAUGUCAGUCUGAGUC | | [1422-1440] |
| 101 | CCUGACUCAGACUGACAUU | AAUGUCAGUCUGAGUCAGG | | [1419-1437] |
| 102 | UUGGAUGACAGAAACACUU | AAGUGUUUCUGUCAUCCAA | Pig | [867-885] |
| 103 | GUGGAGUAUUUGGAUGACA | UGUCAUCCAAAUACUCCAC | | [858-876] |
| 104 | UGGCGCUAAAAGUUUUGAG | CUCAAAACUUUUAGCGCCA | GP | [88-106] |
| 105 | AGACUGGCGCUAAAAGUUU | AAACUUUUAGCGCCAGUCU | GP | [84-102] |
| 106 | GUCGACCUUAGUACCUAAA | UUUAGGUACUAAGGUCGAC | | [2073-2091] |
| 107 | CUUGAAACCACCUUUUAUU | AAUAAAAGGUGGUUUCAAG | | [1896-1914] |
| 108 | UGCAGUUAAGGGUUAGUUU | AAACUAACCCUUAACUGCA | | [1705-1723] |
| 109 | UUCAGACCUAUGGAAACUA | UAGUUUCCAUAGGUCUGAA | MK | [308-326] |
| 110 | CCUGCACUGGUGUUUUGUU | AACAAAACACCAGUGCAGG | | [1597-1615] |
| 111 | CAUUUUCAGACCUAUGGAA | UUCCAUAGGUCUGAAAAUG | MK | [304-322] |
| 112 | GACUGACAUUCUCCACUUC | GAAGUGGAGAAUGUCAGUC | | [1428-1446] |
| 113 | UGACUCAGACUGACAUUCU | AGAAUGUCAGUCUGAGUCA | | [1421-1439] |
| 114 | CACUGGAUGGAGAAUAUUU | AAAUAUUCUCCAUCCAGUG | MK | [1216-1234] |
| 115 | AAAGAAGAAACCACUGGAU | AUCCAGUGGUUUCUUCUUU | MK, GP | [1205-1223] |
| 116 | UCAUCACACUGGAAGACUC | GAGUCUUCCAGUGUGAUGA | RB, Ms | [1012-1030] |
| 117 | UGGAGUAUUUGGAUGACAG | CUGUCAUCCAAAUACUCCA | | [859-877] |
| 118 | GCGUGUGGAGUAUUUGGAU | AUCCAAAUACUCCACACGC | | [854-872] |
| 119 | CUGGCGCUAAAAGUUUUGA | UCAAAACUUUUAGCGCCAG | GP | [87-105] |
| 120 | CCUUCUCCCUUUUUAUAUC | GAUAUAAAAAGGGAGAAGG | | [2565-2583] |
| 121 | AAGCAAUGGAUGAUUUGAU | AUCAAAUCAUCCAUUGCUU | | [364-382] |
| 122 | AGGGAGUUGUCAAGUCUUG | CAAGACUUGACAACUCCCU | | [2020-2038] |
| 123 | CCUUGAAACCACCUUUUAU | AUAAAAGGUGGUUUCAAGG | | [1895-1913] |
| 124 | GCUGUCCCUCAUGUUGAAU | AUUCAACAUGAGGGACAGC | | [1780-1798] |
| 125 | AAAUGUUCUUGCAGUUAAG | CUUAACUGCAAGAACAUUU | | [1696-1714] |
| 126 | AAGAAAUGUUCUUGCAGUU | AACUGCAAGAACAUUUCUU | | [1693-1711] |
| 127 | GUUUGGGAGAUGUAAGAAA | UUUCUUACAUCUCCCAAAC | | [1680-1698] |
| 128 | CUGCACUGGUGUUUUGUUG | CAACAAAACACCAGUGCAG | | [1598-1616] |
| 129 | CAGACUGACAUUCUCCACU | AGUGGAGAAUGUCAGUCUG | | [1426-1444] |
| 130 | UCAGGAAACAUUUUCAGAC | GUCUGAAAAUGUUUCCUGA | MK | [296-314] |
| 131 | GCUGAAUGAGGCCUUGGAA | UUCCAAGGCCUCAUUCAGC | MK, Rat | [1280-1298] |
| 132 | CAAAGAAGAAACCACUGGA | UCCAGUGGUUUCUUCUUUG | MK, GP | [1204-1222] |
| 133 | GCCAAAGAAGAAACCACUG | CAGUGGUUUCUUCUUUGGC | MK, GP | [1202-1220] |
| 134 | AGCCAAAGAAGAAACCACU | AGUGGUUUCUUCUUUGGCU | MK, GP | [1201-1219] |
| 135 | GCUCUGACUGUACCACCAU | AUGGUGGUACAGUCAGAGC | MK, HM, Pig, RB | [928-946] |
| 136 | GAGUAUUUGGAUGACAGAA | UUCUGUCAUCCAAAUACUC | RB | [861-879] |
| 137 | GUGUGGAGUAUUUGGAUGA | UCAUCCAAAUACUCCACAC | | [856-874] |
| 138 | UGGAAGGAAAUUUGCGUGU | ACACGCAAAUUUCCUUCCA | MK | [841-859] |
| 139 | CCCUCCUCAGCAUCUUAUC | GAUAAGAUGCUGAGGAGGG | | [818-836] |
| 140 | ACAGCACAUGACGGAGGUU | AACCUCCGUCAUGUGCUGU | | [749-767] |
| 141 | UCGACCUUAGUACCUAAAA | UUUUAGGUACUAAGGUCGA | | [2074-2092] |
| 142 | GAGUUGUCAAGUCUUGCUG | CAGCAAGACUUGACAACUC | | [2023-2041] |
| 143 | CAGAGUGCAUUGUGAGGGU | ACCCUCACAAUGCACUCUG | | [1854-1872] |
| 144 | AAGGCCCAUAUCUGUGAAA | UUUCACAGAUAUGGGCCUU | | [1812-1830] |
| 145 | UUGCAGUUAAGGGUUAGUU | AACUAACCCUUAACUGCAA | | [1704-1722] |
| 146 | UUCUUGCAGUUAAGGGUUA | UAACCCUUAACUGCAAGAA | | [1701-1719] |
| 147 | AGGGAUGUUUGGGAGAUGU | ACAUCUCCCAAACAUCCCU | | [1674-1692] |
| 148 | GAGGGAUGUUUGGGAGAUG | CAUCUCCCAAACAUCCCUC | | [1673-1691] |
| 149 | CAGGAAACAUUUUCAGACC | GGUCUGAAAAUGUUUCCUG | MK | [297-315] |
| 150 | GCCAUAAAAAACUCAUGUU | AACAUGAGUUUUUUAUGGC | | [1387-1405] |
| 151 | CGUGUGGAGUAUUUGGAUG | CAUCCAAAUACUCCACACG | | [855-873] |
| 152 | UGGAUGAUUUGAUGCUGUC | GACAGCAUCAAAUCAUCCA | MK | [370-388] |
| 153 | CAAUAAAACUUUGCUGCCA | UGGCAGCAAAGUUUUAUUG | | [2611-2629] |
| 154 | CAAUGGAUGAUUUGAUGCU | AGCAUCAAAUCAUCCAUUG | | [367-385] |
| 155 | GAGGGAGUUGUCAAGUCUU | AAGACUUGACAACUCCCUC | | [2019-2037] |
| 156 | UGGUUAGGUAGAGGGAGUU | AACUCCCUCUACCUAACCA | | [2009-2027] |
| 157 | AUGUACAUCUGGCCUUGAA | UUCAAGGCCAGAUGUACAU | | [1883-1901] |
| 158 | UGGGUUUUGGGUCUUUGAA | UUCAAAGACCCAAAACCCA | | [1498-1516] |
| 159 | CCCGCCAUAAAAAACUCAU | AUGAGUUUUUUAUGGCGGG | | [1384-1402] |
| 160 | GAUGUUCCGAGAGCUGAAU | AUUCAGCUCUCGGAACAUC | MK, Pig, Rat | [1268-1286] |
| 161 | AGAUGUUCCGAGAGCUGAA | UUCAGCUCUCGGAACAUCU | MK, Pig, Rat, RB | [1267-1285] |
| 162 | ACAUGUGUAACAGUUCCUG | CAGGAACUGUUACACAUGU | MK, Dog | [958-976] |
| 163 | ACCAUCCACUACAACUACA | UGUAGUUGUAGUGGAUGGU | MK, Dog, GP | [942-960] |
| 164 | GAGCUUCUCAAAAGUCUAG | CUAGACUUUUGAGAAGCUC | | [104-122] |
| 165 | UCGACAUAGUGUGGUGGUG | CACCACCACACUAUGUCGA | MK | [887-905] |
| 166 | GAAGGAAAUUUGCGUGUGG | CCACACGCAAAUUUCCUUC | MK | [843-861] |
| 167 | AUAAUGUACAUCUGGCCUU | AAGGCCAGAUGUACAUUAU | | [1880-1898] |
| 168 | GGGUUUUGGGUCUUUGAAC | GUUCAAAGACCCAAAACCC | | [1499-1517] |
| 169 | GAAGUCCAAAAAGGGUCAG | CUGACCCUUUUUGGACUUC | | [1358-1376] |
| 170 | AGCUUCUCAAAAGUCUAGA | UCUAGACUUUUGAGAAGCU | | [105-123] |
| 171 | GGAAAUUUGCGUGUGGAGU | ACUCCACACGCAAAUUUCC | MK | [846-864] |
| 172 | CUGGUUAGGUAGAGGGAGU | ACUCCCUCUACCUAACCAG | | [2008-2026] |
| 173 | AGCCACCUGAAGUCCAAAA | UUUUGGACUUCAGGUGGCU | | [1350-1368] |
| 174 | CAGAAACACUUUUCGACAU | AUGUCGAAAAGUGUUUCUG | MK | [875-893] |
| 175 | UGCGUGUGGAGUAUUUGGA | UCCAAAUACUCCACACGCA | | [853-871] |
| 176 | GGUAGAGGGAGUUGUCAAG | CUUGACAACUCCCUCUACC | | [2015-2033] |
| 177 | GCCCACUUCACCGUACUAA | UUAGUACGGUGAAGUGGGC | | [1753-1771] |
| 178 | UGGGACGGAACAGCUUUGA | UCAAAGCUGUUCCGUCCCA | MK, HM, Pig, RB | [1045-1063] |
| 179 | GCUUCUUGCAUUCUGGGAC | GUCCCAGAAUGCAAGAAGC | | [586-604] |
| 180 | AUGCUGGCAUUUGCACCUA | UAGGUGCAAAUGCCAGCAU | | [1830-1848] |
| 181 | GGAAACUACUUCCUGAAAA | UUUUCAGGAAGUAGUUUCC | MK, GP | [319-337] |
| 182 | GGAGAAUAUUUCACCCUUC | GAAGGGUGAAAUAUUCUCC | MK, Dog | [1224-1242] |
| 183 | AGAAACACUUUUCGACAUA | UAUGUCGAAAAGUGUUUCU | MK | [876-894] |
| 184 | CAAGAUGUUUUGCCAACUG | CAGUUGGCAAAACAUCUUG | | [644-662] |
| 185 | CUGCCCUCAACAAGAUGUU | AACAUCUUGUUGAGGGCAG | | [634-652] |
| 186 | CGCUAAAAGUUUUGAGCUU | AAGCUCAAAACUUUUAGCG | | [91-109] |
| 187 | UCAGCCACAUUCUAGGUAG | CUACCUAGAAUGUGGCUGA | | [1728-1746] |
| 188 | CCAGGACUUCCAUUUGCUU | AAGCAAAUGGAAGUCCUGG | | [1550-1568] |
| 189 | CCAUAAAAAACUCAUGUUC | GAACAUGAGUUUUUUAUGG | | [1388-1406] |
| 190 | UCCUCACCAUCAUCACACU | AGUGUGAUGAUGGUGAGGA | Pig, RB | [1003-1021] |
| 191 | CCAUCUACAAGCAGUCACA | UGUGACUGCUUGUAGAUGG | MK | [733-751] |
| 192 | CUCUUGGUCGACCUUAGUA | UACUAAGGUCGACCAAGAG | | [2067-2085] |
| 193 | GCCACAUUCUAGGUAGGUA | UACCUACCUAGAAUGUGGC | | [1731-1749] |
| 194 | GGUUUUUACUGUGAGGGAU | AUCCCUCACAGUAAAAACC | | [1661-1679] |
| 195 | CCCAGGACUUCCAUUUGCU | AGCAAAUGGAAGUCCUGGG | | [1549-1567] |
| 196 | CGCCAUAAAAAACUCAUGU | ACAUGAGUUUUUUAUGGCG | | [1386-1404] |
| 197 | CCGCCAUAAAAAACUCAUG | CAUGAGUUUUUUAUGGCGG | | [1385-1403] |
| 198 | GACAGAAACACUUUUCGAC | GUCGAAAAGUGUUUCUGUC | MK, Dog, Pig | [873-891] |
| 199 | CUGGAGGAUUUCAUCUCUU | AAGAGAUGAAAUCCUCCAG | | [2117-2135] |
| 200 | CCCUGUCUGACAACCUCUU | AAGAGGUUGUCAGACAGGG | | [2053-2071] |
| 201 | GUUGGUAGUUUCUACAGUU | AACUGUAGAAACUACCAAC | | [1983-2001] |
| 202 | GGUUGGUAGUUUCUACAGU | ACUGUAGAAACUACCAACC | | [1982-2000] |
| 203 | GGGUUGGUAGUUUCUACAG | CUGUAGAAACUACCAACCC | | [1981-1999] |
| 204 | CCACAUUCUAGGUAGGUAG | CUACCUACCUAGAAUGUGG | | [1732-1750] |
| 205 | GUAGGACAUACCAGCUUAG | CUAAGCUGGUAUGUCCUAC | | [1635-1653] |
| 206 | CCCUGCCAUUUUGGGUUUU | AAAACCCAAAAUGGCAGGG | | [1487-1505] |
| 207 | UCCCGCCAUAAAAAACUCA | UGAGUUUUUUAUGGCGGGA | | [1383-1401] |
| 208 | CUCCCGCCAUAAAAAACUC | GAGUUUUUUAUGGCGGGAG | | [1382-1400] |
| 209 | GGCCUUGGAACUCAAGGAU | AUCCUUGAGUUCCAAGGCC | MK | [1289-1307] |
| 210 | CUCUGAGUCAGGAAACAUU | AAUGUUUCCUGACUCAGAG | MK | [289-307] |
| 211 | UGGAGAAUAUUUCACCCUU | AAGGGUGAAAUAUUCUCCA | MK, Dog, Rat | [1223-1241] |
| 212 | CCUGCCCUCAACAAGAUGU | ACAUCUUGUUGAGGGCAGG | | [633-651] |
| 213 | GAACAAUGGUUCACUGAAG | CUUCAGUGAACCAUUGUUC | | [402-420] |
| 214 | GGCAUUUGCACCUACCUCA | UGAGGUAGGUGCAAAUGCC | | [1835-1853] |
| 215 | UGCUGGCAUUUGCACCUAC | GUAGGUGCAAAUGCCAGCA | | [1831-1849] |
| 216 | CCUGCCAUUUUGGGUUUUG | CAAAACCCAAAAUGGCAGG | | [1488-1506] |
| 217 | GUCUACCUCCCGCCAUAAA | UUUAUGGCGGGAGGUAGAC | RB | [1376-1394] |
| 218 | GGAAGAGAAUCUCCGCAAG | CUUGCGGAGAUUCUCUUCC | | [1106-1124] |
| 219 | ACAACUACAUGUGUAACAG | CUGUUACACAUGUAGUUGU | MK, Dog | [952-970] |
| 220 | GCCAUCUACAAGCAGUCAC | GUGACUGCUUGUAGAUGGC | MK | [732-750] |
| 221 | UCCUUCUCCCUUUUUAUAU | AUAUAAAAAGGGAGAAGGA | | [2564-2582] |
| 222 | CCUCCUUCUCCCUUUUUAU | AUAAAAAGGGAGAAGGAGG | | [2562-2580] |
| 223 | GCCAAACCCUGUCUGACAA | UUGUCAGACAGGGUUUGGC | | [2047-2065] |
| 224 | CAGGACUUCCAUUUGCUUU | AAAGCAAAUGGAAGUCCUG | | [1551-1569] |
| 225 | UGAAGUCCAAAAAGGGUCA | UGACCCUUUUUGGACUUCA | | [1357-1375] |
| 226 | UGAGUCAGGAAACAUUUUC | GAAAAUGUUUCCUGACUCA | MK | [292-310] |
| 227 | GAGAGCUGAAUGAGGCCUU | AAGGCCUCAUUCAGCUCUC | MK, Rat | [1276-1294] |
| 228 | AGAGAAUCUCCGCAAGAAA | UUUCUUGCGGAGAUUCUCU | | [1109-1127] |
| 229 | CACCAUCAUCACACUGGAA | UUCCAGUGUGAUGAUGGUG | Pig, RB | [1007-1025] |
| 230 | CUGCUCAGAUAGCGAUGGU | ACCAUCGCUAUCUGAGCAG | | [794-812] |
| 231 | CUCCUUCUCCCUUUUUAUA | UAUAAAAAGGGAGAAGGAG | | [2563-2581] |
| 232 | UGAUCUGGAUCCACCAAGA | UCUUGGUGGAUCCAGAUCA | | [2144-2162] |
| 233 | CCCACUUCACCGUACUAAC | GUUAGUACGGUGAAGUGGG | | [1754-1772] |
| 234 | GCCUGACUCAGACUGACAU | AUGUCAGUCUGAGUCAGGC | | [1418-1436] |
| 235 | CUGAGUCAGGAAACAUUUU | AAAAUGUUUCCUGACUCAG | MK | [291-309] |
| 236 | AGGAAGAGAAUCUCCGCAA | UUGCGGAGAUUCUCUUCCU | | [1105-1123] |
| 237 | GUGUAACAGUUCCUGCAUG | CAUGCAGGAACUGUUACAC | MK, Dog | [962-980] |
| 238 | AACUACAUGUGUAACAGUU | AACUGUUACACAUGUAGUU | MK, Dog | [954-972] |
| 239 | CAACUACAUGUGUAACAGU | ACUGUUACACAUGUAGUUG | MK, Dog | [953-971] |
| 240 | ACAAGAUGUUUUGCCAACU | AGUUGGCAAAACAUCUUGU | | [643-661] |
| 241 | CGAUAUUGAACAAUGGUUC | GAACCAUUGUUCAAUAUCG | | [395-413] |
| 242 | GUACCUAAAAGGAAAUCUC | GAGAUUUCCUUUUAGGUAC | | [2083-2101] |
| 243 | GUCCCAAGCAAUGGAUGAU | AUCAUCCAUUGCUUGGGAC | | [359-377] |
| 244 | CCACCUUUUAUUACAUGGG | CCCAUGUAAUAAAAGGUGG | | [1903-1921] |
| 245 | GGCCUUGAAACCACCUUUU | AAAAGGUGGUUUCAAGGCC | | [1893-1911] |
| 246 | UCUGUGAAAUGCUGGCAUU | AAUGCCAGCAUUUCACAGA | | [1822-1840] |
| 247 | AUGGAAACUACUUCCUGAA | UUCAGGAAGUAGUUUCCAU | MK, GP | [317-335] |
| 248 | CACCGUACUAACCAGGGAA | UUCCCUGGUUAGUACGGUG | | [1761-1779] |
| 249 | AGGACAUACCAGCUUAGAU | AUCUAAGCUGGUAUGUCCU | | [1637-1655] |
| 250 | UGGCCUGCACUGGUGUUUU | AAAACACCAGUGCAGGCCA | | [1594-1612] |
| 251 | GCUUGCAAUAGGUGUGCGU | ACGCACACCUAUUGCAAGC | | [1522-1540] |
| 252 | CCUCCCGCCAUAAAAAACU | AGUUUUUUAUGGCGGGAGG | | [1381-1399] |
| 253 | CUACCUCCCGCCAUAAAAA | UUUUUAUGGCGGGAGGUAG | MK, RB | [1378-1396] |
| 254 | GAGUCAGGAAACAUUUUCA | UGAAAAUGUUUCCUGACUC | MK | [293-311] |
| 255 | AGAGCUGAAUGAGGCCUUG | CAAGGCCUCAUUCAGCUCU | MK, Rat | [1277-1295] |
| 256 | GGAUGGAGAAUAUUUCACC | GGUGAAAUAUUCUCCAUCC | MK | [1220-1238] |
| 257 | CCACUGGAUGGAGAAUAUU | AAUAUUCUCCAUCCAGUGG | MK | [1215-1233] |
| 258 | AAGAGAAUCUCCGCAAGAA | UUCUUGCGGAGAUUCUCUU | | [1108-1126] |
| 259 | UCCGAGUGGAAGGAAAUUU | AAAUUUCCUUCCACUCGGA | MK, Dog | [835-853] |
| 260 | CAUGAGCGCUGCUCAGAUA | UAUCUGAGCAGCGCUCAUG | | [786-804] |
| 261 | ACAAGCAGUCACAGCACAU | AUGUGCUGUGACUGCUUGU | MK | [739-757] |
| 262 | CUAAAAGUUUUGAGCUUCU | AGAAGCUCAAAACUUUUAG | | [93-111] |
| 263 | CAACAAGAUGUUUUGCCAA | UUGGCAAAACAUCUUGUUG | | [641-659] |
| 264 | UCAACAAGAUGUUUUGCCA | UGGCAAAACAUCUUGUUGA | MK | [640-658] |
| 265 | GCCAAGUCUGUGACUUGCA | UGCAAGUCACAGACUUGGC | MK | [606-624] |
| 266 | GUUUCCGUCUGGGCUUCUU | AAGAAGCCCAGACGGAAAC | | [574-592] |
| 267 | ACACCCUGGAGGAUUUCAU | AUGAAAUCCUCCAGGGUGU | | [2112-2130] |
| 268 | CUGUGAAAUGCUGGCAUUU | AAAUGCCAGCAUUUCACAG | | [1823-1841] |
| 269 | AGGACUUCCAUUUGCUUUG | CAAAGCAAAUGGAAGUCCU | | [1552-1570] |
| 270 | UCAGAAGCACCCAGGACUU | AAGUCCUGGGUGCUUCUGA | | [1540-1558] |
| 271 | AGAAUAUUUCACCCUUCAG | CUGAAGGGUGAAAUAUUCU | MK, Dog | [1226-1244] |
| 272 | AACCACUGGAUGGAGAAUA | UAUUCUCCAUCCAGUGGUU | MK | [1213-1231] |
| 273 | CCAGUGGUAAUCUACUGGG | CCCAGUAGAUUACCACUGG | MK | [1030-1048] |
| 274 | CUCCAGUGGUAAUCUACUG | CAGUAGAUUACCACUGGAG | MK | [1028-1046] |
| 275 | CUGGAAGACUCCAGUGGUA | UACCACUGGAGUCUUCCAG | MK | [1020-1038] |
| 276 | CCAUCAUCACACUGGAAGA | UCUUCCAGUGUGAUGAUGG | Pig, RB, Ms | [1009-1027] |
| 277 | UUAUCCGAGUGGAAGGAAA | UUUCCUUCCACUCGGAUAA | MK, HM | [832-850] |
| 278 | GCAUCUUAUCCGAGUGGAA | UUCCACUCGGAUAAGAUGC | HM | [827-845] |
| 279 | CAAGCAGUCACAGCACAUG | CAUGUGCUGUGACUGCUUG | MK | [740-758] |
| 280 | GUCUGUGACUUGCACGUAC | GUACGUGCAAGUCACAGAC | MK | [611-629] |
| 281 | CCAAGUCUGUGACUUGCAC | GUGCAAGUCACAGACUUGG | MK | [607-625] |
| 282 | GCGCUAAAAGUUUUGAGCU | AGCUCAAAACUUUUAGCGC | GP | [90-108] |
| 283 | UCUGUCCCUUCCCAGAAAA | UUUUCUGGGAAGGGACAGA | MK | [537-555] |
| 284 | UGAAGACCCAGGUCCAGAU | AUCUGGACCUGGGUCUUCA | MK | [416-434] |
| 285 | ACAUUCUGCAAGCACAUCU | AGAUGUGCUUGCAGAAUGU | | [2522-2540] |
| 286 | UCCCAAGCAAUGGAUGAUU | AAUCAUCCAUUGCUUGGGA | | [360-378] |
| 287 | UGGGUUGGUAGUUUCUACA | UGUAGAAACUACCAACCCA | | [1980-1998] |
| 288 | GUGGGUUGGUAGUUUCUAC | GUAGAAACUACCAACCCAC | | [1979-1997] |
| 289 | UCCAAAAAGGGUCAGUCUA | UAGACUGACCCUUUUUGGA | | [1362-1380] |
| 290 | GCUUCGAGAUGUUCCGAGA | UCUCGGAACAUCUCGAAGC | MK, Pig, Rat | [1261-1279] |
| 291 | AGAAACCACUGGAUGGAGA | UCUCCAUCCAGUGGUUUCU | MK | [1210-1228] |
| 292 | GAAGAGAAUCUCCGCAAGA | UCUUGCGGAGAUUCUCUUC | | [1107-1125] |
| 293 | GAACAGCUUUGAGGUGCGU | ACGCACCUCAAAGCUGUUC | Pig | [1052-1070] |
| 294 | CAGUGGUAAUCUACUGGGA | UCCCAGUAGAUUACCACUG | MK | [1031-1049] |
| 295 | GAAGACUCCAGUGGUAAUC | GAUUACCACUGGAGUCUUC | MK | [1023-1041] |
| 296 | CACUUUUCGACAUAGUGUG | CACACUAUGUCGAAAAGUG | MK | [881-899] |
| 297 | UGGCCAUCUACAAGCAGUC | GACUGCUUGUAGAUGGCCA | MK | [730-748] |
| 298 | GGCGCUAAAAGUUUUGAGC | GCUCAAAACUUUUAGCGCC | GP | [89-107] |
| 299 | AGACCCAGGUCCAGAUGAA | UUCAUCUGGACCUGGGUCU | MK | [419-437] |
| 300 | GGAUCCACCAAGACUUGUU | AACAAGUCUUGGUGGAUCC | | [2150-2168] |
| 301 | GGAGGAUUUCAUCUCUUGU | ACAAGAGAUGAAAUCCUCC | | [2119-2137] |
| 302 | UGGAAACUACUUCCUGAAA | UUUCAGGAAGUAGUUUCCA | MK, GP | [318-336] |
| 303 | CCUAUGGAAACUACUUCCU | AGGAAGUAGUUUCCAUAGG | MK | [314-332] |
| 304 | CCACUGAACAAGUUGGCCU | AGGCCAACUUGUUCAGUGG | | [1581-1599] |
| 305 | UGAACCCUUGCUUGCAAUA | UAUUGCAAGCAAGGGUUCA | | [1513-1531] |
| 306 | GGUCUUUGAACCCUUGCUU | AAGCAAGGGUUCAAAGACC | | [1507-1525] |
| 307 | CAGCUUUGAGGUGCGUGUU | AACACGCACCUCAAAGCUG | Pig | [1055-1073] |
| 308 | ACCAUCAUCACACUGGAAG | CUUCCAGUGUGAUGAUGGU | Pig, RB, Ms | [1008-1026] |
| 309 | UCACCAUCAUCACACUGGA | UCCAGUGUGAUGAUGGUGA | Pig, RB | [1006-1024] |
| 310 | CACUACAACUACAUGUGUA | UACACAUGUAGUUGUAGUG | MK, Dog | [948-966] |
| 311 | AUAGUGUGGUGGUGCCCUA | UAGGGCACCACCACACUAU | MK | [892-910] |
| 312 | ACAGCCAAGUCUGUGACUU | AAGUCACAGACUUGGCUGU | MK | [603-621] |
| 313 | UUCUGUCCCUUCCCAGAAA | UUUCUGGGAAGGGACAGAA | | [536-554] |
| 314 | UCUGGAUCCACCAAGACUU | AAGUCUUGGUGGAUCCAGA | | [2147-2165] |
| 315 | CCCUGGAGGAUUUCAUCUC | GAGAUGAAAUCCUCCAGGG | | [2115-2133] |
| 316 | CUUAGUACCUAAAAGGAAA | UUUCCUUUUAGGUACUAAG | | [2079-2097] |
| 317 | GGUCGACCUUAGUACCUAA | UUAGGUACUAAGGUCGACC | | [2072-2090] |
| 318 | CCAAACCCUGUCUGACAAC | GUUGUCAGACAGGGUUUGG | | [2048-2066] |
| 319 | CUGAAAACAACGUUCUGUC | GACAGAACGUUGUUUUCAG | GP | [331-349] |
| 320 | GGAGUAGGACAUACCAGCU | AGCUGGUAUGUCCUACUCC | | [1632-1650] |
| 321 | UGCAAUAGGUGUGCGUCAG | CUGACGCACACCUAUUGCA | | [1525-1543] |
| 322 | UGCUCAAGACUGGCGCUAA | UUAGCGCCAGUCUUGAGCA | | [78-96] |
| 323 | GCCAUUUUGGGUUUUGGGU | ACCCAAAACCCAAAAUGGC | | [1491-1509] |
| 324 | CCCUCUGAGUCAGGAAACA | UGUUUCCUGACUCAGAGGG | MK | [287-305] |
| 325 | CCAAAGAAGAAACCACUGG | CCAGUGGUUUCUUCUUUGG | MK, GP | [1203-1221] |
| 326 | AGAAAACCUACCAGGGCAG | CUGCCCUGGUAGGUUUUCU | | [550-568] |
| 327 | UGAACAAUGGUUCACUGAA | UUCAGUGAACCAUUGUUCA | | [401-419] |
| 328 | GAGCUGGAAGGGUCAACAU | AUGUUGACCCUUCCAGCUC | | [2498-2516] |
| 329 | GAUCCACCAAGACUUGUUU | AAACAAGUCUUGGUGGAUC | | [2151-2169] |
| 330 | ACCUCUUGGUCGACCUUAG | CUAAGGUCGACCAAGAGGU | | [2065-2083] |
| 331 | ACCUCACAGAGUGCAUUGU | ACAAUGCACUCUGUGAGGU | | [1848-1866] |
| 332 | CUACCUCACAGAGUGCAUU | AAUGCACUCUGUGAGGUAG | | [1846-1864] |
| 333 | GUACUAACCAGGGAAGCUG | CAGCUUCCCUGGUUAGUAC | | [1765-1783] |
| 334 | GGGUCUUUGAACCCUUGCU | AGCAAGGGUUCAAAGACCC | | [1506-1524] |
| 335 | CUCAUGUUCAAGACAGAAG | CUUCUGUCUUGAACAUGAG | | [1398-1416] |
| 336 | AGCUGAAUGAGGCCUUGGA | UCCAAGGCCUCAUUCAGCU | MK, Rat | [1279-1297] |
| 337 | ACUACAACUACAUGUGUAA | UUACACAUGUAGUUGUAGU | MK, Dog | [949-967] |
| 338 | UGGUCGACCUUAGUACCUA | UAGGUACUAAGGUCGACCA | | [2071-2089] |
| 339 | UGGCCUUGAAACCACCUUU | AAAGGUGGUUUCAAGGCCA | | [1892-1910] |
| 340 | CUGGCCUUGAAACCACCUU | AAGGUGGUUUCAAGGCCAG | | [1891-1909] |
| 341 | GCCUGCACUGGUGUUUUGU | ACAAAACACCAGUGCAGGC | | [1596-1614] |
| 342 | UGAACAAGUUGGCCUGCAC | GUGCAGGCCAACUUGUUCA | | [1585-1603] |
| 343 | ACCCUUGCUUGCAAUAGGU | ACCUAUUGCAAGCAAGGGU | | [1516-1534] |
| 344 | CCAGCCAAAGAAGAAACCA | UGGUUUCUUCUUUGGCUGG | MK | [1199-1217] |
| 345 | CCCAGCCAAAGAAGAAACC | GGUUUCUUCUUUGGCUGGG | MK | [1198-1216] |
| 346 | AGCUUUGAGGUGCGUGUUU | AAACACGCACCUCAAAGCU | Pig | [1056-1074] |
| 347 | UCACACUGGAAGACUCCAG | CUGGAGUCUUCCAGUGUGA | MK, RB, Ms | [1015-1033] |
| 348 | GCUUCUCAAAAGUCUAGAG | CUCUAGACUUUUGAGAAGC | | [106-124] |
| 349 | CUCUGACUGUACCACCAUC | GAUGGUGGUACAGUCAGAG | MK, HM, Pig, RB | [929-947] |
| 350 | AGCAUCUUAUCCGAGUGGA | UCCACUCGGAUAAGAUGCU | HM | [826-844] |
| 351 | UCAGCAUCUUAUCCGAGUG | CACUCGGAUAAGAUGCUGA | HM | [824-842] |
| 352 | AAAGUUUUGAGCUUCUCAA | UUGAGAAGCUCAAAACUUU | | [96-114] |
| 353 | AGCAGUCACAGCACAUGAC | GUCAUGUGCUGUGACUGCU | MK | [742-760] |
| 354 | GGCCAUCUACAAGCAGUCA | UGACUGCUUGUAGAUGGCC | MK | [731-749] |
| 355 | GCUGUGGGUUGAUUCCACA | UGUGGAAUCAACCCACAGC | | [683-701] |
| 356 | AGCUGUGGGUUGAUUCCAC | GUGGAAUCAACCCACAGCU | | [682-700] |
| 357 | UCCCUUCCCAGAAAACCUA | UAGGUUUUCUGGGAAGGGA | MK | [541-559] |
| 358 | CCUACCUCACAGAGUGCAU | AUGCACUCUGUGAGGUAGG | | [1845-1863] |
| 359 | ACCGUACUAACCAGGGAAG | CUUCCCUGGUUAGUACGGU | | [1762-1780] |
| 360 | UCACCGUACUAACCAGGGA | UCCCUGGUUAGUACGGUGA | | [1760-1778] |
| 361 | CUGAACAAGUUGGCCUGCA | UGCAGGCCAACUUGUUCAG | | [1584-1602] |
| 362 | GCCUUGGAACUCAAGGAUG | CAUCCUUGAGUUCCAAGGC | MK | [1290-1308] |
| 363 | CAGCCAAAGAAGAAACCAC | GUGGUUUCUUCUUUGGCUG | MK, GP | [1200-1218] |
| 364 | ACAGCUUUGAGGUGCGUGU | ACACGCACCUCAAAGCUGU | Pig | [1054-1072] |
| 365 | UCCACUACAACUACAUGUG | CACAUGUAGUUGUAGUGGA | MK, Dog, GP | [946-964] |
| 366 | UGAGCUUCUCAAAAGUCUA | UAGACUUUUGAGAAGCUCA | | [103-121] |
| 367 | CUCAGCAUCUUAUCCGAGU | ACUCGGAUAAGAUGCUGAG | HM | [823-841] |
| 368 | CAAGCACAUCUGCAUUUUC | GAAAAUGCAGAUGUGCUUG | | [2530-2548] |
| 369 | UGCAAGCACAUCUGCAUUU | AAAUGCAGAUGUGCUUGCA | | [2528-2546] |
| 370 | CCUUAGUACCUAAAAGGAA | UUCCUUUUAGGUACUAAGG | | [2078-2096] |
| 371 | UAAGAAAUGUUCUUGCAGU | ACUGCAAGAACAUUUCUUA | | [1692-1710] |
| 372 | CCCUUGCUUGCAAUAGGUG | CACCUAUUGCAAGCAAGGG | | [1517-1535] |
| 373 | GUUUUGAGCUUCUCAAAAG | CUUUUGAGAAGCUCAAAAC | | [99-117] |
| 374 | UCCUCAGCAUCUUAUCCGA | UCGGAUAAGAUGCUGAGGA | HM | [821-839] |
| 375 | CUCACAGAGUGCAUUGUGA | UCACAAUGCACUCUGUGAG | | [1850-1868] |
| 376 | UGUAAGAAAUGUUCUUGCA | UGCAAGAACAUUUCUUACA | | [1690-1708] |
| 377 | GGCUCCACUGAACAAGUUG | CAACUUGUUCAGUGGAGCC | | [1577-1595] |
| 378 | UCGAGAUGUUCCGAGAGCU | AGCUCUCGGAACAUCUCGA | MK, Pig, Rat, RB | [1264-1282] |
| 379 | CAACCUCUUGGUCGACCUU | AAGGUCGACCAAGAGGUUG | | [2063-2081] |
| 380 | UUGAACCCUUGCUUGCAAU | AUUGCAAGCAAGGGUUCAA | | [1512-1530] |
| 381 | UCCAGAUGAAGCUCCCAGA | UCUGGGAGCUUCAUCUGGA | | [428-446] |
| 382 | ACAACCUCUUGGUCGACCU | AGGUCGACCAAGAGGUUGU | | [2062-2080] |
| 383 | GGCUUCUUGCAUUCUGGGA | UCCCAGAAUGCAAGAAGCC | | [585-603] |
| 384 | UCUGCAAGCACAUCUGCAU | AUGCAGAUGUGCUUGCAGA | | [2526-2544] |
| 385 | CUGACAACCUCUUGGUCGA | UCGACCAAGAGGUUGUCAG | | [2059-2077] |
| 386 | CUGCAAGCACAUCUGCAUU | AAUGCAGAUGUGCUUGCAG | | [2527-2545] |
| 387 | CUGGGCUUCUUGCAUUCUG | CAGAAUGCAAGAAGCCCAG | | [582-600] |
| 388 | CAGAAAACCUACCAGGGCA | UGCCCUGGUAGGUUUUCUG | | [549-567] |
| 389 | GUCUGGGCUUCUUGCAUUC | GAAUGCAAGAAGCCCAGAC | | [580-598] |
| 390 | CCUCUGAGUCAGGAAACAU | AUGUUUCCUGACUCAGAGG | MK | [288-306] |
| 391 | AGGCCUUGGAACUCAAGGA | UCCUUGAGUUCCAAGGCCU | MK | [1288-1306] |

TABLE B2
P53 19-mer oligomer pairs - tumor protein p53

| | | | | Human- |
|---|---|---|---|---|
| | | | | 8400737: |
| No. | Sense | AntiSense | Other Sp | 252-1433 |

| | | | | |
|---|---|---|---|---|
| 1 | CAUUGUGAGGGUUAAUGAA | UUCAUUAACCCUCACAAUG | | [1861-1879] |
| 2 | GCUCAAGACUGGCGCUAAA | UUUAGCGCCAGUCUUGAGC | | [79-97] |
| 3 | GUUAAGGGUUAGUUUACAA | UUGUAAACUAACCCUUAAC | | [1709-1727] |
| 4 | GCAGUUAAGGGUUAGUUUA | UAAACUAACCCUUAACUGC | | [1706-1724] |
| 5 | GAAUUUUCUCUAACUUCAA | UUGAAGUUAGAGAAAAUUC | | [1795-1813] |
| 6 | GAAAUGUUCUUGCAGUUAA | UUAACUGCAAGAACAUUUC | | [1695-1713] |
| 7 | GGGUUAAUGAAAUAAUGUA | UACAUUAUUUCAUUAACCC | | [1869-1887] |
| 8 | AUACCAGCUUAGAUUUUAA | UUAAAAUCUAAGCUGGUAU | | [1642-1660] |
| 9 | CAAUAGGUGUGCGUCAGAA | UUCUGACGCACACCUAUUG | | [1527-1545] |
| 10 | GGAUUUCAUCUCUUGUAUA | UAUACAAGAGAUGAAAUCC | | [2122-2140] |
| 11 | CAUGUUGAAUUUUCUCUAA | UUAGAGAAAAUUCAACAUG | | [1789-1807] |
| 12 | GUACCACCAUCCACUACAA | UUGUAGUGGAUGGUGGUAC | Pig, Mnk | [937-955] |
| 13 | CGAUCUCUUAUUUUACAAU | AUUGUAAAAUAAGAGAUCG | | [2596-2614] |
| 14 | GGAAGACUCCAGUGGUAAU | AUUACCACUGGAGUCUUCC | Mnk | [1022-1040] |
| 15 | UGUGAGGGUUAAUGAAAUA | UAUUUCAUUAACCCUCACA | | [1864-1882] |
| 16 | GGACAUACCAGCUUAGAUU | AAUCUAAGCUGGUAUGUCC | | [1638-1656] |
| 17 | AUAAAAAACUCAUGUUCAA | UUGAACAUGAGUUUUUUAU | | [1390-1408] |
| 18 | GAGGGUUAAUGAAAUAAUG | CAUUAUUUCAUUAACCCUC | | [1867-1885] |
| 19 | GAUCUCUUAUUUUACAAUA | UAUUGUAAAAUAAGAGAUC | | [2597-2615] |
| 20 | GAAACCACUGGAUGGAGAA | UUCUCCAUCCAGUGGUUUC | Mnk | [1211-1229] |
| 21 | AGUUAAGGGUUAGUUUACA | UGUAAACUAACCCUUAACU | | [1708-1726] |
| 22 | AACUCAUGUUCAAGACAGA | UCUGUCUUGAACAUGAGUU | | [1396-1414] |
| 23 | GAAUAUUUCACCCUUCAGA | UCUGAAGGGUGAAAUAUUC | Dog, Mnk | [1227-1245] |
| 24 | AUAUCGAUCUCUUAUUUUA | UAAAAUAAGAGAUCGAUAU | | [2592-2610] |
| 25 | CUGGAUGGAGAAUAUUUCA | UGAAAUAUUCUCCAUCCAG | Rb, Mnk | [1218-1236] |
| 26 | UGUACAUCUGGCCUUGAAA | UUUCAAGGCCAGAUGUACA | | [1884-1902] |
| 27 | GAAAUUUGCGUGUGGAGUA | UACUCCACACGCAAAUUUC | Mnk | [847-865] |
| 28 | UCUACCUCCCGCCAUAAAA | UUUUAUGGCGGGAGGUAGA | Rb, Mnk | [1377-1395] |
| 29 | UGUAUAUGAUGAUCUGGAU | AUCCAGAUCAUCAUAUACA | | [2135-2153] |
| 30 | AGGUAGAGGGAGUUGUCAA | UUGACAACUCCCUCUACCU | | [2014-2032] |
| 31 | CCACCAUCCACUACAACUA | UAGUUGUAGUGGAUGGUGG | GP, Dog, Mnk | [940-958] |
| 32 | GAAGGGUCAACAUCUUUUA | UAAAAGAUGUUGACCCUUC | | [2504-2522] |
| 33 | GCGCACAGAGGAAGAGAAU | AUUCUCUUCCUCUGUGCGC | Mnk | [1097-1115] |
| 34 | CCAGAUGAAGCUCCCAGAA | UUCUGGGAGCUUCAUCUGG | | [429-447] |
| 35 | UGAGGGUUAAUGAAAUAAU | AUUAUUUCAUUAACCCUCA | | [1866-1884] |
| 36 | UACCUCCCGCCAUAAAAAA | UUUUUUAUGGCGGGAGGUA | Rb, Mnk | [1379-1397] |
| 37 | UCGAUCUCUUAUUUUACAA | UUGUAAAAUAAGAGAUCGA | | [2595-2613] |
| 38 | CAAGGCCCAUAUCUGUGAA | UUCACAGAUAUGGGCCUUG | | [1811-1829] |
| 39 | GAGGUUGGCUCUGACUGUA | UACAGUCAGAGCCAACCUC | Hamster, Rb, Mnk | [921-939] |
| 40 | CAGACCUAUGGAAACUACU | AGUAGUUUCCAUAGGUCUG | Mnk | [310-328] |
| 41 | GAAACCACCUUUUAUUACA | UGUAAUAAAAGGUGGUUUC | | [1899-1917] |
| 42 | GCUUAGAUUUUAAGGUUUU | AAAACCUUAAAAUCUAAGC | | [1648-1666] |
| 43 | CAUACCAGCUUAGAUUUUA | UAAAAUCUAAGCUGGUAUG | | [1641-1659] |
| 44 | AAGGGUUAGUUUACAAUCA | UGAUUGUAAACUAACCCUU | | [1712-1730] |
| 45 | CAAGCAAUGGAUGAUUUGA | UCAAAUCAUCCAUUGCUUG | | [363-381] |
| 46 | CAGCCACCUGAAGUCCAAA | UUUGGACUUCAGGUGGCUG | | [1349-1367] |
| 47 | GUUAGUUUACAAUCAGCCA | UGGCUGAUUGUAAACUAAC | | [1716-1734] |
| 48 | CUGUGAGGGAUGUUUGGGA | UCCCAAACAUCCCUCACAG | | [1669-1687] |
| 49 | UGAAUUUUCUCUAACUUCA | UGAAGUUAGAGAAAAUUCA | | [1794-1812] |
| 50 | UUUUUAUAUCGAUCUCUUA | UAAGAGAUCGAUAUAAAAA | | [2587-2605] |
| 51 | CGUCCCAAGCAAUGGAUGA | UCAUCCAUUGCUUGGGACG | | [358-376] |
| 52 | GAAACUACUUCCUGAAAAC | GUUUUCAGGAAGUAGUUUC | GP, Mnk | [320-338] |
| 53 | CAUCCUCACCAUCAUCACA | UGUGAUGAUGGUGAGGAUG | Rb, Pig | [1001-1019] |
| 54 | CAAGACUGGCGCUAAAAGU | ACUUUUAGCGCCAGUCUUG | | [82-100] |
| 55 | GUCAGGAAACAUUUUCAGA | UCUGAAAAUGUUUCCUGAC | Mnk | [295-313] |
| 56 | GAGUGCAUUGUGAGGGUUA | UAACCCUCACAAUGCACUC | | [1856-1874] |
| 57 | AGUCUACCUCCCGCCAUAA | UUAUGGCGGGAGGUAGACU | Rb | [1375-1393] |
| 58 | AUUGUGAGGGUUAAUGAAA | UUUCAUUAACCCUCACAAU | | [1862-1880] |
| 59 | CAUAAAAAACUCAUGUUCA | UGAACAUGAGUUUUUUAUG | | [1389-1407] |
| 60 | CAUCUUUUACAUUCUGCAA | UUGCAGAAUGUAAAAGAUG | | [2514-2532] |
| 61 | GAUUUCAUCUCUUGUAUAU | AUAUACAAGAGAUGAAAUC | | [2123-2141] |
| 62 | UAUGGAAACUACUUCCUGA | UCAGGAAGUAGUUUCCAUA | GP, Mnk | [316-334] |
| 63 | CAUCUUCUGUCCCUUCCCA | UGGGAAGGGACAGAAGAUG | | [532-550] |
| 64 | CUGGAAGGGUCAACAUCUU | AAGAUGUUGACCCUUCCAG | | [2501-2519] |
| 65 | AUGUUUGGGAGAUGUAAGA | UCUUACAUCUCCCAAACAU | | [1678-1696] |
| 66 | CUUUGAGGUGCGUGUUUGU | ACAAACACGCACCUCAAAG | Pig | [1058-1076] |
| 67 | CUUUUUAUAUCCCAUUUUU | AAAAAUGGGAUAUAAAAAG | | [2573-2591] |
| 68 | AACUACUUCCUGAAAACAA | UUGUUUUCAGGAAGUAGUU | GP | [322-340] |
| 69 | AGGAUUUCAUCUCUUGUAU | AUACAAGAGAUGAAAUCCU | | [2121-2139] |
| 70 | ACUACUUCCUGAAAACAAC | GUUGUUUUCAGGAAGUAGU | GP | [323-341] |
| 71 | GUAUUUGGAUGACAGAAAC | GUUUCUGUCAUCCAAAUAC | Rb | [863-881] |
| 72 | UUUGGGAGAUGUAAGAAAU | AUUUCUUACAUCUCCCAAA | | [1681-1699] |
| 73 | CUCAGACUGACAUUCUCCA | UGGAGAAUGUCAGUCUGAG | | [1424-1442] |
| 74 | UGACAGAAACACUUUUCGA | UCGAAAAGUGUUUCUGUCA | Pig, Mnk | [872-890] |
| 75 | AGUAGGACAUACCAGCUUA | UAAGCUGGUAUGUCCUACU | | [1634-1652] |
| 76 | UCAUGUUGAAUUUUCUCUA | UAGAGAAAAUUCAACAUGA | | [1788-1806] |
| 77 | AAUAUUUCACCCUUCAGAU | AUCUGAAGGGUGAAAUAUU | Dog, Mnk | [1228-1246] |
| 78 | AUCGAUCUCUUAUUUUACA | UGUAAAAUAAGAGAUCGAU | | [2594-2612] |
| 79 | GAUGUUUGGGAGAUGUAAG | CUUACAUCUCCCAAACAUC | | [1677-1695] |
| 80 | AGCUGGUUAGGUAGAGGGA | UCCCUCUACCUAACCAGCU | | [2006-2024] |
| 81 | ACAGAAACACUUUUCGACA | UGUCGAAAAGUGUUUCUGU | Pig, Dog, Mnk | [874-892] |
| 82 | CAGUUAAGGGUUAGUUUAC | GUAAACUAACCCUUAACUG | | [1707-1725] |
| 83 | UGAUGAUCUGGAUCCACCA | UGGUGGAUCCAGAUCAUCA | | [2141-2159] |
| 84 | ACAUUUUCAGACCUAUGGA | UCCAUAGGUCUGAAAAUGU | Mnk | [303-321] |
| 85 | CAUUUGCACCUACCUCACA | UGUGAGGUAGGUGCAAAUG | | [1837-1855] |
| 86 | CUUAGAUUUUAAGGUUUUU | AAAAACCUUAAAAUCUAAG | | [1649-1667] |
| 87 | AGAUUUUAAGGUUUUUACU | AGUAAAAACCUUAAAAUCU | | [1652-1670] |
| 88 | CGUCUGGGCUUCUUGCAUU | AAUGCAAGAAGCCCAGACG | | [579-597] |
| 89 | GCAAUAGGUGUGCGUCAGA | UCUGACGCACACCUAUUGC | | [1526-1544] |
| 90 | UACAAUCAGCCACAUUCUA | UAGAAUGUGGCUGAUUGUA | | [1723-1741] |
| 91 | AGGUUUUUACUGUGAGGGA | UCCCUCACAGUAAAAACCU | | [1660-1678] |
| 92 | CCUGAAGUCCAAAAAGGGU | ACCCUUUUUGGACUUCAGG | | [1355-1373] |
| 93 | GUGAGGGAUGUUUGGGAGA | UCUCCCAAACAUCCCUCAC | | [1671-1689] |
| 94 | AUCCCAUUUUUAUAUCGAU | AUCGAUAUAAAAAUGGGAU | | [2581-2599] |
| 95 | UUGGGUUUUGGGUCUUUGA | UCAAAGACCCAAAACCCAA | | [1497-1515] |
| 96 | GACAUACCAGCUUAGAUUU | AAAUCUAAGCUGGUAUGUC | | [1639-1657] |
| 97 | UGUUUUGCCAACUGGCCAA | UUGGCCAGUUGGCAAAACA | | [649-667] |
| 98 | GGUAAUCUACUGGGACGGA | UCCGUCCCAGUAGAUUACC | Mnk | [1035-1053] |
| 99 | GUUAAUGAAAUAAUGUACA | UGUACAUUAUUUCAUUAAC | | [1871-1889] |
| 100 | UCUCUAACUUCAAGGCCCA | UGGGCCUUGAAGUUAGAGA | | [1801-1819] |
| 101 | AUGAGGCCUUGGAACUCAA | UUGAGUUCCAAGGCCUCAU | Mnk | [1285-1303] |
| 102 | UGUUUGGGAGAUGUAAGAA | UUCUUACAUCUCCCAAACA | | [1679-1697] |
| 103 | UGGUGGGUUGGUAGUUUCU | AGAAACUACCAACCCACCA | | [1977-1995] |
| 104 | AAAUUUGCGUGUGGAGUAU | AUACUCCACACGCAAAUUU | Mnk | [848-866] |
| 105 | UAGUUUACAAUCAGCCACA | UGUGGCUGAUUGUAAACUA | | [1718-1736] |
| 106 | AGAGGGAGUUGUCAAGUCU | AGACUUGACAACUCCCUCU | | [2018-2036] |
| 107 | AGCACAUGACGGAGGUUGU | ACAACCUCCGUCAUGUGCU | | [751-769] |
| 108 | GAGGAAGAGAAUCUCCGCA | UGCGGAGAUUCUCUUCCUC | | [1104-1122] |
| 109 | AAGGAAAUUUGCGUGUGGA | UCCACACGCAAAUUUCCUU | Mnk | [844-862] |
| 110 | ACUGUACCACCAUCCACUA | UAGUGGAUGGUGGUACAGU | Hamster, Rb, Pig, Mnk | [934-952] |
| 111 | UCUGAGUCAGGAAACAUUU | AAAUGUUUCCUGACUCAGA | Mnk | [290-308] |
| 112 | GGUUAAUGAAAUAAUGUAC | GUACAUUAUUUCAUUAACC | | [1870-1888] |
| 113 | GAGAUGUUCCGAGAGCUGA | UCAGCUCUCGGAACAUCUC | Rat, Rb, Pig, Mnk | [1266-1284] |
| 114 | AGCUUAGAUUUUAAGGUUU | AAACCUUAAAAUCUAAGCU | | [1647-1665] |
| 115 | AACAAUGGUUCACUGAAGA | UCUUCAGUGAACCAUUGUU | | [403-421] |
| 116 | UACCUAAAAGGAAAUCUCA | UGAGAUUUCCUUUUAGGUA | | [2084-2102] |
| 117 | AAACCACCUUUUAUUACAU | AUGUAAUAAAAGGUGGUUU | | [1900-1918] |
| 118 | UUGUCAUGGCGACUGUCCA | UGGACAGUCGCCAUGACAA | | [3-21] |
| 119 | CCAUGGCCAUCUACAAGCA | UGCUUGUAGAUGGCCAUGG | Mnk | [727-745] |
| 120 | GAGGAUUUCAUCUCUUGUA | UACAAGAGAUGAAAUCCUC | | [2120-2138] |
| 121 | UUGGGCAGCUGGUUAGGUA | UACCUAACCAGCUGCCCAA | | [2000-2018] |
| 122 | GACUGUACCACCAUCCACU | AGUGGAUGGUGGUACAGUC | Hamster, Rb, Pig, Mnk | [933-951] |
| 123 | GUUUACAAUCAGCCACAUU | AAUGUGGCUGAUUGUAAAC | | [1720-1738] |
| 124 | UGAAAUGCUGGCAUUUGCA | UGCAAAUGCCAGCAUUUCA | | [1826-1844] |
| 125 | ACCACUGGAUGGAGAAUAU | AUAUUCUCCAUCCAGUGGU | Mnk | [1214-1232] |
| 126 | GAUGAUCUGGAUCCACCAA | UUGGUGGAUCCAGAUCAUC | | [2142-2160] |
| 127 | AUUUUCAGACCUAUGGAAA | UUUCCAUAGGUCUGAAAAU | Mnk | [305-323] |
| 128 | AAGUUUUGAGCUUCUCAAA | UUUGAGAAGCUCAAAACUU | | [97-115] |
| 129 | AGGGUUAAUGAAAUAAUGU | ACAUUAUUUCAUUAACCCU | | [1868-1886] |
| 130 | AAUCUACUGGGACGGAACA | UGUUCCGUCCCAGUAGAUU | Mnk | [1038-1056] |
| 131 | AGACUCCAGUGGUAAUCUA | UAGAUUACCACUGGAGUCU | Mnk | [1025-1043] |
| 132 | GAGAAUCUCCGCAAGAAAG | CUUUCUUGCGGAGAUUCUC | | [1110-1128] |
| 133 | CAUUUUGGGUUUUGGGUCU | AGACCCAAAACCCAAAAUG | | [1493-1511] |
| 134 | UAUUUGGAUGACAGAAACA | UGUUUCUGUCAUCCAAAUA | Rb | [864-882] |
| 135 | UAUCCGAGUGGAAGGAAAU | AUUUCCUUCCACUCGGAUA | Hamster, Mnk | [833-851] |
| 136 | CUUCUUGCAUUCUGGGACA | UGUCCCAGAAUGCAAGAAG | | [587-605] |
| 137 | AAAAAACUCAUGUUCAAGA | UCUUGAACAUGAGUUUUUU | | [1392-1410] |
| 138 | AUUUUUAUAUCGAUCUCUU | AAGAGAUCGAUAUAAAAAU | | [2586-2604] |
| 139 | UUUACAUUCUGCAAGCACA | UGUGCUUGCAGAAUGUAAA | | [2519-2537] |
| 140 | GUUGAAUUUUCUCUAACUU | AAGUUAGAGAAAAUUCAAC | | [1792-1810] |
| 141 | GCACAGAGGAAGAGAAUCU | AGAUUCUCUUCCUCUGUGC | | [1099-1117] |
| 142 | GGAGAUGUAAGAAAUGUUC | GAACAUUUCUUACAUCUCC | | [1685-1703] |
| 143 | UGUGCAGCUGUGGGUUGAU | AUCAACCCACAGCUGCACA | | [677-695] |
| 144 | AGUGGAAGGAAAUUUGCGU | ACGCAAAUUUCCUUCCACU | Mnk | [839-857] |
| 145 | CCUUUUUAUAUCCCAUUUU | AAAAUGGGAUAUAAAAAGG | | [2572-2590] |
| 146 | UAUCGAUCUCUUAUUUUAC | GUAAAAUAAGAGAUCGAUA | | [2593-2611] |
| 147 | CGAGUGGAAGGAAAUUUGC | GCAAAUUUCCUUCCACUCG | Dog, Mnk | [837-855] |
| 148 | UUUGGAUGACAGAAACACU | AGUGUUUCUGUCAUCCAAA | | [866-884] |
| 149 | CUUCAAGGCCCAUAUCUGU | ACAGAUAUGGGCCUUGAAG | | [1808-1826] |
| 150 | AUAUGAUGAUCUGGAUCCA | UGGAUCCAGAUCAUCAUAU | | [2138-2156] |
| 151 | UGUUCCGAGAGCUGAAUGA | UCAUUCAGCUCUCGGAACA | Rat, Pig, Mnk | [1270-1288] |
| 152 | GAACAAGUUGGCCUGCACU | AGUGCAGGCCAACUUGUUC | | [1586-1604] |
| 153 | GAGAUGUAAGAAAUGUUCU | AGAACAUUUCUUACAUCUC | | [1686-1704] |
| 154 | CUUCCCAGAAAACCUACCA | UGGUAGGUUUUCUGGGAAG | Mnk | [544-562] |
| 155 | GAUAUUGAACAAUGGUUCA | UGAACCAUUGUUCAAUAUC | | [396-414] |
| 156 | AGACUGACAUUCUCCACUU | AAGUGGAGAAUGUCAGUCU | | [1427-1445] |
| 157 | ACUCAUGUUCAAGACAGAA | UUCUGUCUUGAACAUGAGU | | [1397-1415] |
| 158 | UCAACAUCUUUUACAUUCU | AGAAUGUAAAAGAUGUUGA | | [2510-2528] |
| 159 | AGUUUUGAGCUUCUCAAAA | UUUUGAGAAGCUCAAAACU | | [98-116] |
| 160 | UUAAGGGUUAGUUUACAAU | AUUGUAAACUAACCCUUAA | | [1710-1728] |
| 161 | AGCCAAACCCUGUCUGACA | UGUCAGACAGGGUUUGGCU | | [2046-2064] |
| 162 | UCAUGUUCAAGACAGAAGG | CCUUCUGUCUUGAACAUGA | | [1399-1417] |
| 163 | ACAUGACGGAGGUUGUGAG | CUCACAACCUCCGUCAUGU | | [754-772] |
| 164 | UUGUAUAUGAUGAUCUGGA | UCCAGAUCAUCAUAUACAA | | [2134-2152] |
| 165 | AAGACUGGCGCUAAAAGUU | AACUUUUAGCGCCAGUCUU | | [83-101] |
| 166 | CUUUUACAUUCUGCAAGCA | UGCUUGCAGAAUGUAAAAG | | [2517-2535] |
| 167 | AGCAAUGGAUGAUUUGAUG | CAUCAAAUCAUCCAUUGCU | | [365-383] |
| 168 | CACUUCACCGUACUAACCA | UGGUUAGUACGGUGAAGUG | | [1756-1774] |
| 169 | GGACUUCCAUUUGCUUUGU | ACAAAGCAAAUGGAAGUCC | | [1553-1571] |
| 170 | AAUAGGUGUGCGUCAGAAG | CUUCUGACGCACACCUAUU | | [1528-1546] |
| 171 | AGGAAAUUUGCGUGUGGAG | CUCCACACGCAAAUUUCCU | Mnk | [845-863] |
| 172 | CAUUCUGCAAGCACAUCUG | CAGAUGUGCUUGCAGAAUG | | [2523-2541] |
| 173 | UUUUAUAUCGAUCUCUUAU | AUAAGAGAUCGAUAUAAAA | | [2588-2606] |
| 174 | CAACAUCUUUUACAUUCUG | CAGAAUGUAAAAGAUGUUG | | [2511-2529] |
| 175 | AAGCACAUCUGCAUUUUCA | UGAAAAUGCAGAUGUGCUU | | [2531-2549] |
| 176 | GACGGAACAGCUUUGAGGU | ACCUCAAAGCUGUUCCGUC | Hamster, Rb, Pig, Mnk | [1048-1066] |
| 177 | GACGAUAUUGAACAAUGGU | ACCAUUGUUCAAUAUCGUC | | [393-411] |
| 178 | GGGCUCCACUGAACAAGUU | AACUUGUUCAGUGGAGCCC | | [1576-1594] |
| 179 | GGGAGUUGUCAAGUCUUGC | GCAAGACUUGACAACUCCC | | [2021-2039] |
| 180 | AUUUGCGUGUGGAGUAUUU | AAAUACUCCACACGCAAAU | | [850-868] |
| 181 | UUACAUUCUGCAAGCACAU | AUGUGCUUGCAGAAUGUAA | | [2520-2538] |
| 182 | UAUCCCAUUUUUAUAUCGA | UCGAUAUAAAAAUGGGAUA | | [2580-2598] |
| 183 | CCUGGAGGAUUUCAUCUCU | AGAGAUGAAAUCCUCCAGG | | [2116-2134] |
| 184 | CUCUAACUUCAAGGCCCAU | AUGGGCCUUGAAGUUAGAG | | [1802-1820] |
| 185 | UUGAAACCACCUUUUAUUA | UAAUAAAAGGUGGUUUCAA | | [1897-1915] |
| 186 | ACUCCAGUGGUAAUCUACU | AGUAGAUUACCACUGGAGU | Mnk | [1027-1045] |
| 187 | GUGCAGCUGUGGGUUGAUU | AAUCAACCCACAGCUGCAC | | [678-696] |
| 188 | GUCCAAAAAGGGUCAGUCU | AGACUGACCCUUUUUGGAC | | [1361-1379] |
| 189 | AAACCUACCAGGGCAGCUA | UAGCUGCCCUGGUAGGUUU | | [553-571] |
| 190 | GACAUUCUCCACUUCUUGU | ACAAGAAGUGGAGAAUGUC | | [1432-1450] |
| 191 | UUACAAUCAGCCACAUUCU | AGAAUGUGGCUGAUUGUAA | | [1722-1740] |
| 192 | UCAAGACUGGCGCUAAAAG | CUUUUAGCGCCAGUCUUGA | | [81-99] |
| 193 | UUAUAUCGAUCUCUUAUUU | AAAUAAGAGAUCGAUAUAA | | [2590-2608] |
| 194 | UCUUCUGUCCCUUCCCAGA | UCUGGGAAGGGACAGAAGA | | [534-552] |
| 195 | UGUGUAACAGUUCCUGCAU | AUGCAGGAACUGUUACACA | Dog, Mnk | [961-979] |
| 196 | CCCUUCCCAGAAAACCUAC | GUAGGUUUUCUGGGAAGGG | Mnk | [542-560] |
| 197 | AGAUGUAAGAAAUGUUCUU | AAGAACAUUUCUUACAUCU | | [1687-1705] |
| 198 | UACAAGCAGUCACAGCACA | UGUGCUGUGACUGCUUGUA | Mnk | [738-756] |
| 199 | CCCAAGCAAUGGAUGAUUU | AAAUCAUCCAUUGCUUGGG | | [361-379] |
| 200 | GGAAGCUGUCCCUCAUGUU | AACAUGAGGGACAGCUUCC | | [1776-1794] |
| 201 | AAGCUGUCCCUCAUGUUGA | UCAACAUGAGGGACAGCUU | | [1778-1796] |
| 202 | UAGUUUCUACAGUUGGGCA | UGCCCAACUGUAGAAACUA | | [1988-2006] |
| 203 | UGACACGCUUCCCUGGAUU | AAUCCAGGGAAGCGUGUCA | | [203-221] |
| 204 | UGUUCUUGCAGUUAAGGGU | ACCCUUAACUGCAAGAACA | | [1699-1717] |
| 205 | GCACUGGUGUUUUGUUGUG | CACAACAAAACACCAGUGC | | [1600-1618] |
| 206 | CUGACUGUACCACCAUCCA | UGGAUGGUGGUACAGUCAG | Hamster, Rb, Pig, Mnk | [931-949] |
| 207 | AAACUACUUCCUGAAAACA | UGUUUUCAGGAAGUAGUUU | GP | [321-339] |
| 208 | CAUCCACUACAACUACAUG | CAUGUAGUUGUAGUGGAUG | GP, Dog, Mnk | [944-962] |
| 209 | GCUGGAAGGGUCAACAUCU | AGAUGUUGACCCUUCCAGC | | [2500-2518] |
| 210 | CUUCUGUCCCUUCCCAGAA | UUCUGGGAAGGGACAGAAG | | [535-553] |
| 211 | UUUGCACCUACCUCACAGA | UCUGUGAGGUAGGUGCAAA | | [1839-1857] |
| 212 | UGAGGGAUGUUUGGGAGAU | AUCUCCCAAACAUCCCUCA | | [1672-1690] |
| 213 | UAGGACAUACCAGCUUAGA | UCUAAGCUGGUAUGUCCUA | | [1636-1654] |
| 214 | AAGCAGUCACAGCACAUGA | UCAUGUGCUGUGACUGCUU | Mnk | [741-759] |
| 215 | CCACUACAACUACAUGUGU | ACACAUGUAGUUGUAGUGG | Dog, Mnk | [947-965] |
| 216 | GAGUGGAAGGAAAUUUGCG | CGCAAAUUUCCUUCCACUC | Dog, Mnk | [838-856] |
| 217 | ACUGGAUGGAGAAUAUUUC | GAAAUAUUCUCCAUCCAGU | Mnk | [1217-1235] |
| 218 | UGCAUUCUGGGACAGCCAA | UUGGCUGUCCCAGAAUGCA | | [592-610] |
| 219 | AUCUCUUGUAUAUGAUGAU | AUCAUCAUAUACAAGAGAU | | [2129-2147] |
| 220 | CCUCUUGGUCGACCUUAGU | ACUAAGGUCGACCAAGAGG | | [2066-2084] |
| 221 | GCUUUGAGGUGCGUGUUUG | CAAACACGCACCUCAAAGC | Pig | [1057-1075] |
| 222 | UAAUCUACUGGGACGGAAC | GUUCCGUCCCAGUAGAUUA | Mnk | [1037-1055] |
| 223 | AAACAUUUUCAGACCUAUG | CAUAGGUCUGAAAAUGUUU | Mnk | [301-319] |
| 224 | CUACAAGCAGUCACAGCAC | GUGCUGUGACUGCUUGUAG | Mnk | [737-755] |
| 225 | UGUGAGGGAUGUUUGGGAG | CUCCCAAACAUCCCUCACA | | [1670-1688] |
| 226 | ACAGUUGGGCAGCUGGUUA | UAACCAGCUGCCCAACUGU | | [1996-2014] |
| 227 | CAAUCAGCCACAUUCUAGG | CCUAGAAUGUGGCUGAUUG | | [1725-1743] |
| 228 | GGUAGUUUCUACAGUUGGG | CCCAACUGUAGAAACUACC | | [1986-2004] |
| 229 | UCUCCCUUUUUAUAUCCCA | UGGGAUAUAAAAAGGGAGA | | [2568-2586] |
| 230 | GCACCCAGGACUUCCAUUU | AAAUGGAAGUCCUGGGUGC | | [1546-1564] |
| 231 | UGGAAGGGUCAACAUCUUU | AAAGAUGUUGACCCUUCCA | | [2502-2520] |
| 232 | UGGGAGAUGUAAGAAAUGU | ACAUUUCUUACAUCUCCCA | | [1683-1701] |
| 233 | AGUCACAGCACAUGACGGA | UCCGUCAUGUGCUGUGACU | Ms, GP, Mnk | [745-763] |
| 234 | AGUCAGGAAACAUUUUCAG | CUGAAAAUGUUUCCUGACU | Mnk | [294-312] |
| 235 | GUAAGAAAUGUUCUUGCAG | CUGCAAGAACAUUUCUUAC | | [1691-1709] |
| 236 | UAUAUCGAUCUCUUAUUUU | AAAAUAAGAGAUCGAUAUA | | [2591-2609] |
| 237 | UGAGGUUGGCUCUGACUGU | ACAGUCAGAGCCAACCUCA | Hamster, Mnk | [920-938] |
| 238 | GUUCUUGCAGUUAAGGGUU | AACCCUUAACUGCAAGAAC | | [1700-1718] |
| 239 | AUGGUUCACUGAAGACCCA | UGGGUCUUCAGUGAACCAU | | [407-425] |
| 240 | CUUGCAGUUAAGGGUUAGU | ACUAACCCUUAACUGCAAG | | [1703-1721] |
| 241 | UGUUGAAUUUUCUCUAACU | AGUUAGAGAAAAUUCAACA | | [1791-1809] |
| 242 | UUGCAAUAGGUGUGCGUCA | UGACGCACACCUAUUGCAA | | [1524-1542] |
| 243 | CUGGCAUUUGCACCUACCU | AGGUAGGUGCAAAUGCCAG | | [1833-1851] |
| 244 | CUUUGAACCCUUGCUUGCA | UGCAAGCAAGGGUUCAAAG | | [1510-1528] |
| 245 | UCUCAAAAGUCUAGAGCCA | UGGCUCUAGACUUUUGAGA | | [109-127] |
| 246 | UAGGUAGAGGGAGUUGUCA | UGACAACUCCCUCUACCUA | | [2013-2031] |
| 247 | CAUCUCUUGUAUAUGAUGA | UCAUCAUAUACAAGAGAUG | | [2128-2146] |
| 248 | GAAAUAAUGUACAUCUGGC | GCCAGAUGUACAUUAUUUC | | [1877-1895] |
| 249 | UGACAUUCUCCACUUCUUG | CAAGAAGUGGAGAAUGUCA | | [1431-1449] |
| 250 | AAUUUUCUCUAACUUCAAG | CUUGAAGUUAGAGAAAAUU | | [1796-1814] |
| 251 | GAAGAAACCACUGGAUGGA | UCCAUCCAGUGGUUUCUUC | Mnk | [1208-1226] |
| 252 | UAGUGUGGUGGUGCCCUAU | AUAGGGCACCACCACACUA | Mnk | [893-911] |
| 253 | CAACAACACCAGCUCCUCU | AGAGGAGCUGGUGUUGUUG | Mnk | [1178-1196] |
| 254 | GCAAGCACAUCUGCAUUUU | AAAAUGCAGAUGUGCUUGC | | [2529-2547] |
| 255 | CAUUUUUAUAUCGAUCUCU | AGAGAUCGAUAUAAAAAUG | | [2585-2603] |
| 256 | CACCAUCCACUACAACUAC | GUAGUUGUAGUGGAUGGUG | GP, Dog, Mnk | [941-959] |
| 257 | AGUCAGAUCCUAGCGUCGA | UCGACGCUAGGAUCUGACU | | [265-283] |
| 258 | UCUACAAGCAGUCACAGCA | UGCUGUGACUGCUUGUAGA | Mnk | [736-754] |
| 259 | CUGAAUGAGGCCUUGGAAC | GUUCCAAGGCCUCAUUCAG | Mnk | [1281-1299] |
| 260 | AGUGGUAAUCUACUGGGAC | GUCCCAGUAGAUUACCACU | Mnk | [1032-1050] |
| 261 | UGUGGAGUAUUUGGAUGAC | GUCAUCCAAAUACUCCACA | | [857-875] |
| 262 | CUUAUCCGAGUGGAAGGAA | UUCCUUCCACUCGGAUAAG | Hamster, Mnk | [831-849] |
| 263 | AUUUUAAGGUUUUUACUGU | ACAGUAAAAACCUUAAAAU | | [1654-1672] |
| 264 | GAGUAGGACAUACCAGCUU | AAGCUGGUAUGUCCUACUC | | [1633-1651] |
| 265 | CGCACAGAGGAAGAGAAUC | GAUUCUCUUCCUCUGUGCG | | [1098-1116] |
| 266 | AGAGGAAGAGAAUCUCCGC | GCGGAGAUUCUCUUCCUCU | | [1103-1121] |
| 267 | AAAAGUCUAGAGCCACCGU | ACGGUGGCUCUAGACUUUU | | [113-131] |
| 268 | AUGGAUGAUUUGAUGCUGU | ACAGCAUCAAAUCAUCCAU | | [369-387] |
| 269 | UUUAUAUCGAUCUCUUAUU | AAUAAGAGAUCGAUAUAAA | | [2589-2607] |
| 270 | AUGAAAUAAUGUACAUCUG | CAGAUGUACAUUAUUUCAU | | [1875-1893] |
| 271 | UCAGACCUAUGGAAACUAC | GUAGUUUCCAUAGGUCUGA | Mnk | [309-327] |
| 272 | AAGACAGAAGGGCCUGACU | AGUCAGGCCCUUCUGUCUU | | [1407-1425] |
| 273 | UGGAAGACUCCAGUGGUAA | UUACCACUGGAGUCUUCCA | Mnk | [1021-1039] |
| 274 | UCAAGGCCCAUAUCUGUGA | UCACAGAUAUGGGCCUUGA | | [1810-1828] |
| 275 | UUAGUACCUAAAAGGAAAU | AUUUCCUUUUAGGUACUAA | | [2080-2098] |
| 276 | ACAAUCAGCCACAUUCUAG | CUAGAAUGUGGCUGAUUGU | | [1724-1742] |
| 277 | AAGCACCCAGGACUUCCAU | AUGGAAGUCCUGGGUGCUU | | [1544-1562] |
| 278 | CAUUCUGGGACAGCCAAGU | ACUUGGCUGUCCCAGAAUG | | [594-612] |
| 279 | UGAAUGAGGCCUUGGAACU | AGUUCCAAGGCCUCAUUCA | Hamster, Mnk | [1282-1300] |
| 280 | CUACUUCCUGAAAACAACG | CGUUGUUUUCAGGAAGUAG | GP | [324-342] |
| 281 | GAUUUUAAGGUUUUUACUG | CAGUAAAAACCUUAAAAUC | | [1653-1671] |
| 282 | GUCCCUUCCCAGAAAACCU | AGGUUUUCUGGGAAGGGAC | Mnk | [540-558] |
| 283 | ACUGGUGUUUUGUUGUGGG | CCCACAACAAAACACCAGU | | [1602-1620] |
| 284 | GUGGUAAUCUACUGGGACG | CGUCCCAGUAGAUUACCAC | Mnk | [1033-1051] |
| 285 | UACUGUGAGGGAUGUUUGG | CCAAACAUCCCUCACAGUA | | [1667-1685] |
| 286 | GUAGAGGGAGUUGUCAAGU | ACUUGACAACUCCCUCUAC | | [2016-2034] |
| 287 | AUGUUUUGCCAACUGGCCA | UGGCCAGUUGGCAAAACAU | | [648-666] |
| 288 | AUGUGUAACAGUUCCUGCA | UGCAGGAACUGUUACACAU | Dog, Mnk | [960-978] |
| 289 | CCAAGCAAUGGAUGAUUUG | CAAAUCAUCCAUUGCUUGG | | [362-380] |
| 290 | ACCUUAGUACCUAAAAGGA | UCCUUUUAGGUACUAAGGU | | [2077-2095] |
| 291 | GUACAUCUGGCCUUGAAAC | GUUUCAAGGCCAGAUGUAC | | [1885-1903] |
| 292 | UCUGUGACUUGCACGUACU | AGUACGUGCAAGUCACAGA | Mnk | [612-630] |
| 293 | CACACCCUGGAGGAUUUCA | UGAAAUCCUCCAGGGUGUG | | [2111-2129] |
| 294 | AGUCUGUGACUUGCACGUA | UACGUGCAAGUCACAGACU | Mnk | [610-628] |
| 295 | AAACCACUGGAUGGAGAAU | AUUCUCCAUCCAGUGGUUU | Mnk | [1212-1230] |
| 296 | UGAAGCUCCCAGAAUGCCA | UGGCAUUCUGGGAGCUUCA | | [434-452] |
| 297 | UCAAGACAGAAGGGCCUGA | UCAGGCCCUUCUGUCUUGA | | [1405-1423] |
| 298 | UUUCAUCUCUUGUAUAUGA | UCAUAUACAAGAGAUGAAA | | [2125-2143] |
| 299 | UUUAAGGUUUUUACUGUGA | UCACAGUAAAAACCUUAAA | | [1656-1674] |
| 300 | AGCACCCAGGACUUCCAUU | AAUGGAAGUCCUGGGUGCU | | [1545-1563] |
| 301 | CAGAUGAAGCUCCCAGAAU | AUUCUGGGAGCUUCAUCUG | | [430-448] |
| 302 | CAUGUUCAAGACAGAAGGG | CCCUUCUGUCUUGAACAUG | | [1400-1418] |
| 303 | GUUAGGUAGAGGGAGUUGU | ACAACUCCCUCUACCUAAC | | [2011-2029] |
| 304 | ACUGGGACGGAACAGCUUU | AAAGCUGUUCCGUCCCAGU | Mnk | [1043-1061] |
| 305 | CACCCUGGAGGAUUUCAUC | GAUGAAAUCCUCCAGGGUG | | [2113-2131] |
| 306 | CACCUGAAGUCCAAAAAGG | CCUUUUUGGACUUCAGGUG | | [1353-1371] |
| 307 | CUGUCCCUUCCCAGAAAAC | GUUUUCUGGGAAGGGACAG | Mnk | [538-556] |
| 308 | AAUGAAAUAAUGUACAUCU | AGAUGUACAUUAUUUCAUU | | [1874-1892] |
| 309 | AGAAUCUCCGCAAGAAAGG | CCUUUCUUGCGGAGAUUCU | | [1111-1129] |
| 310 | CCUCAUGUUGAAUUUUCUC | GAGAAAAUUCAACAUGAGG | | [1786-1804] |
| 311 | UAGAUUUUAAGGUUUUUAC | GUAAAAACCUUAAAAUCUA | | [1651-1669] |
| 312 | UUUGAACCCUUGCUUGCAA | UUGCAAGCAAGGGUUCAAA | | [1511-1529] |
| 313 | GAAUCUCCGCAAGAAAGGG | CCCUUUCUUGCGGAGAUUC | | [1112-1130] |
| 314 | GGAUGAUUUGAUGCUGUCC | GGACAGCAUCAAAUCAUCC | | [371-389] |
| 315 | CUCAGAUAGCGAUGGUCUG | CAGACCAUCGCUAUCUGAG | | [797-815] |
| 316 | GCUCAGAUAGCGAUGGUCU | AGACCAUCGCUAUCUGAGC | | [796-814] |
| 317 | UGCACUGGUGUUUUGUUGU | ACAACAAAACACCAGUGCA | | [1599-1617] |
| 318 | AGAUGAAGCUCCCAGAAUG | CAUUCUGGGAGCUUCAUCU | | [431-449] |
| 319 | CCUUCCCAGAAAACCUACC | GGUAGGUUUUCUGGGAAGG | Mnk | [543-561] |
| 320 | UGGAGGAUUUCAUCUCUUG | CAAGAGAUGAAAUCCUCCA | | [2118-2136] |
| 321 | CACCCAGGACUUCCAUUUG | CAAAUGGAAGUCCUGGGUG | | [1547-1565] |
| 322 | UAAGGUUUUUACUGUGAGG | CCUCACAGUAAAAACCUUA | | [1658-1676] |
| 323 | ACUUCAAGGCCCAUAUCUG | CAGAUAUGGGCCUUGAAGU | | [1807-1825] |
| 324 | GGUUAGUUUACAAUCAGCC | GGCUGAUUGUAAACUAACC | | [1715-1733] |
| 325 | CUAUGGAAACUACUUCCUG | CAGGAAGUAGUUUCCAUAG | Mnk | [315-333] |
| 326 | UUGGGAGAUGUAAGAAAUG | CAUUUCUUACAUCUCCCAA | | [1682-1700] |
| 327 | AAUUUGCGUGUGGAGUAUU | AAUACUCCACACGCAAAUU | Mnk | [849-867] |
| 328 | CUGGAUCCACCAAGACUUG | CAAGUCUUGGUGGAUCCAG | | [2148-2166] |
| 329 | GUUGGCUCUGACUGUACCA | UGGUACAGUCAGAGCCAAC | Hamster, Rb, Mnk | [924-942] |
| 330 | UGAAAUAAUGUACAUCUGG | CCAGAUGUACAUUAUUUCA | | [1876-1894] |
| 331 | UACCAGCUUAGAUUUUAAG | CUUAAAAUCUAAGCUGGUA | | [1643-1661] |
| 332 | AAAACUCAUGUUCAAGACA | UGUCUUGAACAUGAGUUUU | | [1394-1412] |
| 333 | GUGAAAUGCUGGCAUUUGC | GCAAAUGCCAGCAUUUCAC | | [1825-1843] |
| 334 | UACAACUACAUGUGUAACA | UGUUACACAUGUAGUUGUA | Dog, Mnk | [951-969] |
| 335 | CACAUGACGGAGGUUGUGA | UCACAACCUCCGUCAUGUG | | [753-771] |
| 336 | ACCUGAAGUCCAAAAAGGG | CCCUUUUUGGACUUCAGGU | | [1354-1372] |
| 337 | GGUUAGGUAGAGGGAGUUG | CAACUCCCUCUACCUAACC | | [2010-2028] |
| 338 | UAAGGGUUAGUUUACAAUC | GAUUGUAAACUAACCCUUA | | [1711-1729] |
| 339 | AAAAGUUUUGAGCUUCUCA | UGAGAAGCUCAAAACUUUU | | [95-113] |
| 340 | AUGGCCAUCUACAAGCAGU | ACUGCUUGUAGAUGGCCAU | Mnk | [729-747] |
| 341 | CACUGGUGUUUUGUUGUGG | CCACAACAAAACACCAGUG | | [1601-1619] |
| 342 | CAGCCAAGUCUGUGACUUG | CAAGUCACAGACUUGGCUG | Mnk | [604-622] |
| 343 | CACAUUCUAGGUAGGUAGG | CCUACCUACCUAGAAUGUG | | [1733-1751] |
| 344 | UACUGGGACGGAACAGCUU | AAGCUGUUCCGUCCCAGUA | Mnk | [1042-1060] |
| 345 | GAUGGAGAAUAUUUCACCC | GGGUGAAAUAUUCUCCAUC | Rat, Dog, Mnk | [1221-1239] |
| 346 | UUUUCAGACCUAUGGAAAC | GUUUCCAUAGGUCUGAAAA | Mnk | [306-324] |
| 347 | GUUCAAGACAGAAGGGCCU | AGGCCCUUCUGUCUUGAAC | | [1403-1421] |
| 348 | AGGUUGGCUCUGACUGUAC | GUACAGUCAGAGCCAACCU | Hamster, Rb, Mnk | [922-940] |
| 349 | ACUUCACCGUACUAACCAG | CUGGUUAGUACGGUGAAGU | | [1757-1775] |
| 350 | CACACUGGAAGACUCCAGU | ACUGGAGUCUUCCAGUGUG | Ms, Rb, Mnk | [1016-1034] |
| 351 | GUUUUUACUGUGAGGGAUG | CAUCCCUCACAGUAAAAAC | | [1662-1680] |
| 352 | ACCCUGUCUGACAACCUCU | AGAGGUUGUCAGACAGGGU | | [2052-2070] |
| 353 | CAUCUUAUCCGAGUGGAAG | CUUCCACUCGGAUAAGAUG | Hamster | [828-846] |
| 354 | ACACUUUUCGACAUAGUGU | ACACUAUGUCGAAAAGUGU | Mnk | [880-898] |
| 355 | AAUGUACAUCUGGCCUUGA | UCAAGGCCAGAUGUACAUU | | [1882-1900] |
| 356 | UUUGGGUCUUUGAACCCUU | AAGGGUUCAAAGACCCAAA | | [1503-1521] |
| 357 | ACCACCAUCCACUACAACU | AGUUGUAGUGGAUGGUGGU | Pig, GP, Dog, Mnk | [939-957] |
| 358 | AUUUGCACCUACCUCACAG | CUGUGAGGUAGGUGCAAAU | | [1838-1856] |
| 359 | CAGCUGUGGGUUGAUUCCA | UGGAAUCAACCCACAGCUG | | [681-699] |
| 360 | AGCACAUCUGCAUUUUCAC | GUGAAAAUGCAGAUGUGCU | | [2532-2550] |
| 361 | UAGAGGGAGUUGUCAAGUC | GACUUGACAACUCCCUCUA | | [2017-2035] |
| 362 | CAUGGCCAUCUACAAGCAG | CUGCUUGUAGAUGGCCAUG | Mnk | [728-746] |
| 363 | AGUUGUCAAGUCUUGCUGG | CCAGCAAGACUUGACAACU | | [2024-2042] |
| 364 | CCUUGCUUGCAAUAGGUGU | ACACCUAUUGCAAGCAAGG | | [1518-1536] |
| 365 | AACCCUGUCUGACAACCUC | GAGGUUGUCAGACAGGGUU | | [2051-2069] |
| 366 | GGUUUUGGGUCUUUGAACC | GGUUCAAAGACCCAAAACC | | [1500-1518] |
| 367 | CAGCCACAUUCUAGGUAGG | CCUACCUAGAAUGUGGCUG | | [1729-1747] |
| 368 | AAACUCAUGUUCAAGACAG | CUGUCUUGAACAUGAGUUU | | [1395-1413] |
| 369 | CCUGUCUGACAACCUCUUG | CAAGAGGUUGUCAGACAGG | | [2054-2072] |
| 370 | GCCCAUAUCUGUGAAAUGC | GCAUUUCACAGAUAUGGGC | | [1815-1833] |
| 371 | GAAAUGCUGGCAUUUGCAC | GUGCAAAUGCCAGCAUUUC | | [1827-1845] |
| 372 | AAAAACUCAUGUUCAAGAC | GUCUUGAACAUGAGUUUUU | | [1393-1411] |
| 373 | CAUUCUCCACUUCUUGUUC | GAACAAGAAGUGGAGAAUG | | [1434-1452] |
| 374 | CUGUGACUUGCACGUACUC | GAGUACGUGCAAGUCACAG | Mnk | [613-631] |
| 375 | CAGAGGAAGAGAAUCUCCG | CGGAGAUUCUCUUCCUCUG | | [1102-1120] |
| 376 | UCUUUUACAUUCUGCAAGC | GCUUGCAGAAUGUAAAAGA | | [2516-2534] |
| 377 | UUUUGGGUUUUGGGUCUUU | AAAGACCCAAAACCCAAAA | | [1495-1513] |
| 378 | UGGGCUUCUUGCAUUCUGG | CCAGAAUGCAAGAAGCCCA | | [583-601] |
| 379 | UACAGUUGGGCAGCUGGUU | AACCAGCUGCCCAACUGUA | | [1995-2013] |
| 380 | AAACCCUGUCUGACAACCU | AGGUUGUCAGACAGGGUUU | | [2050-2068] |
| 381 | UGGAACUCAAGGAUGCCCA | UGGGCAUCCUUGAGUUCCA | Mnk | [1294-1312] |
| 382 | CUUCGAGAUGUUCCGAGAG | CUCUCGGAACAUCUCGAAG | Rat, Pig, Mnk | [1262-1280] |
| 383 | CCUUGGAACUCAAGGAUGC | GCAUCCUUGAGUUCCAAGG | Mnk | [1291-1309] |
| 384 | AAGGGUCAACAUCUUUUAC | GUAAAAGAUGUUGACCCUU | | [2505-2523] |
| 385 | UACUAACCAGGGAAGCUGU | ACAGCUUCCCUGGUUAGUA | | [1766-1784] |
| 386 | CUGCCAUUUUGGGUUUUGG | CCAAAACCCAAAAUGGCAG | | [1489-1507] |
| 387 | UCUUGCAGUUAAGGGUUAG | CUAACCCUUAACUGCAAGA | | [1702-1720] |
| 388 | UUCUGCAAGCACAUCUGCA | UGCAGAUGUGCUUGCAGAA | | [2525-2543] |
| 389 | UAAAAAACUCAUGUUCAAG | CUUGAACAUGAGUUUUUUA | | [1391-1409] |
| 390 | UUGCGUGUGGAGUAUUUGG | CCAAAUACUCCACACGCAA | | [852-870] |
| 391 | CAAAAAGGGUCAGUCUACC | GGUAGACUGACCCUUUUUG | | [1364-1382] |
| 392 | AAGACCCAGGUCCAGAUGA | UCAUCUGGACCUGGGUCUU | Mnk | [418-436] |
| 393 | UUAAGGUUUUUACUGUGAG | CUCACAGUAAAAACCUUAA | | [1657-1675] |
| 394 | UAGGUGUGCGUCAGAAGCA | UGCUUCUGACGCACACCUA | | [1530-1548] |
| 395 | GUUCCGAGAGCUGAAUGAG | CUCAUUCAGCUCUCGGAAC | Rat, Mnk | [1271-1289] |
| 396 | CGUACUAACCAGGGAAGCU | AGCUUCCCUGGUUAGUACG | | [1764-1782] |
| 397 | CUUCCUGAAAACAACGUUC | GAACGUUGUUUUCAGGAAG | GP | [327-345] |
| 398 | GACUCCAGUGGUAAUCUAC | GUAGAUUACCACUGGAGUC | Mnk | [1026-1044] |
| 399 | AGAUGUUUUGCCAACUGGC | GCCAGUUGGCAAAACAUCU | | [646-664] |
| 400 | UUUUAAGGUUUUUACUGUG | CACAGUAAAAACCUUAAAA | | [1655-1673] |
| 401 | CAUCUACAAGCAGUCACAG | CUGUGACUGCUUGUAGAUG | Mnk | [734-752] |
| 402 | UGCAGCUGUGGGUUGAUUC | GAAUCAACCCACAGCUGCA | | [679-697] |
| 403 | AAGCUCCCAGAAUGCCAGA | UCUGGCAUUCUGGGAGCUU | | [436-454] |
| 404 | AAGAAGAAACCACUGGAUG | CAUCCAGUGGUUUCUUCUU | GP, Mnk | [1206-1224] |
| 405 | ACCAAGACUUGUUUUAUGC | GCAUAAAACAAGUCUUGGU | | [2156-2174] |
| 406 | UCCUGAAAACAACGUUCUG | CAGAACGUUGUUUUCAGGA | GP | [329-347] |
| 407 | AAUGGAUGAUUUGAUGCUG | CAGCAUCAAAUCAUCCAUU | | [368-386] |
| 408 | UCAAAAGUCUAGAGCCACC | GGUGGCUCUAGACUUUUGA | | [111-129] |
| 409 | UCAGACUGACAUUCUCCAC | GUGGAGAAUGUCAGUCUGA | | [1425-1443] |
| 410 | GUGGAAGGAAAUUUGCGUG | CACGCAAAUUUCCUUCCAC | Mnk | [840-858] |
| 411 | UGUCAUCUUCUGUCCCUUC | GAAGGGACAGAAGAUGACA | | [529-547] |
| 412 | GAUCUGGAUCCACCAAGAC | GUCUUGGUGGAUCCAGAUC | | [2145-2163] |
| 413 | CUUCCAUUUGCUUUGUCCC | GGGACAAAGCAAAUGGAAG | | [1556-1574] |
| 414 | UUUUUACUGUGAGGGAUGU | ACAUCCCUCACAGUAAAAA | | [1663-1681] |
| 415 | UCUUAUCCGAGUGGAAGGA | UCCUUCCACUCGGAUAAGA | Hamster | [830-848] |
| 416 | CCUCCUCAGCAUCUUAUCC | GGAUAAGAUGCUGAGGAGG | Hamster | [819-837] |
| 417 | AGAAGAAACCACUGGAUGG | CCAUCCAGUGGUUUCUUCU | Mnk | [1207-1225] |
| 418 | CUUGUAUAUGAUGAUCUGG | CCAGAUCAUCAUAUACAAG | | [2133-2151] |
| 419 | CUGUCUGACAACCUCUUGG | CCAAGAGGUUGUCAGACAG | | [2055-2073] |
| 420 | CCUCAGCAUCUUAUCCGAG | CUCGGAUAAGAUGCUGAGG | Hamster | [822-840] |
| 421 | UUCAUCUCUUGUAUAUGAU | AUCAUAUACAAGAGAUGAA | | [2126-2144] |
| 422 | UUAGGUAGAGGGAGUUGUC | GACAACUCCCUCUACCUAA | | [2012-2030] |
| 423 | CAUAUCUGUGAAAUGCUGG | CCAGCAUUUCACAGAUAUG | | [1818-1836] |
| 424 | UUUACAAUCAGCCACAUUC | GAAUGUGGCUGAUUGUAAA | | [1721-1739] |
| 425 | UGUACCACCAUCCACUACA | UGUAGUGGAUGGUGGUACA | Pig, Mnk | [936-954] |
| 426 | GUCUUUGAACCCUUGCUUG | CAAGCAAGGGUUCAAAGAC | | [1508-1526] |
| 427 | UGGAUCCACCAAGACUUGU | ACAAGUCUUGGUGGAUCCA | | [2149-2167] |
| 428 | AUGGCGACUGUCCAGCUUU | AAAGCUGGACAGUCGCCAU | GP | [8-26] |
| 429 | AUUUCACCCUUCAGAUCCG | CGGAUCUGAAGGGUGAAAU | Dog, Mnk | [1231-1249] |
| 430 | UGCCAUUUUGGGUUUUGGG | CCCAAAACCCAAAAUGGCA | | [1490-1508] |
| 431 | ACAUCUGGCCUUGAAACCA | UGGUUUCAAGGCCAGAUGU | | [1887-1905] |
| 432 | UUUUGCCAACUGGCCAAGA | UCUUGGCCAGUUGGCAAAA | | [651-669] |
| 433 | ACCUAAAAGGAAAUCUCAC | GUGAGAUUUCCUUUUAGGU | | [2085-2103] |
| 434 | UGAGGCCUUGGAACUCAAG | CUUGAGUUCCAAGGCCUCA | Mnk | [1286-1304] |
| 435 | AAUGAGGCCUUGGAACUCA | UGAGUUCCAAGGCCUCAUU | Mnk | [1284-1302] |
| 436 | UUACUGUGAGGGAUGUUUG | CAAACAUCCCUCACAGUAA | | [1666-1684] |
| 437 | ACUGGCGCUAAAAGUUUUG | CAAAACUUUUAGCGCCAGU | GP | [86-104] |
| 438 | UGUAACAGUUCCUGCAUGG | CCAUGCAGGAACUGUUACA | Dog, Mnk | [963-981] |
| 439 | UUUUGAGCUUCUCAAAAGU | ACUUUUGAGAAGCUCAAAA | | [100-118] |
| 440 | UUGCAUUCUGGGACAGCCA | UGGCUGUCCCAGAAUGCAA | | [591-609] |
| 441 | CAUCUGGCCUUGAAACCAC | GUGGUUUCAAGGCCAGAUG | | [1888-1906] |
| 442 | ACUGGAAGACUCCAGUGGU | ACCACUGGAGUCUUCCAGU | Mnk | [1019-1037] |
| 443 | GUCAACAUCUUUUACAUUC | GAAUGUAAAAGAUGUUGAC | | [2509-2527] |
| 444 | GACAGCCAAGUCUGUGACU | AGUCACAGACUUGGCUGUC | | [602-620] |
| 445 | UAAUGUACAUCUGGCCUUG | CAAGGCCAGAUGUACAUUA | | [1881-1899] |
| 446 | AAGUCUGUGACUUGCACGU | ACGUGCAAGUCACAGACUU | Mnk | [609-627] |
| 447 | GAUGAUUUGAUGCUGUCCC | GGGACAGCAUCAAAUCAUC | | [372-390] |
| 448 | ACUGACAUUCUCCACUUCU | AGAAGUGGAGAAUGUCAGU | | [1429-1447] |
| 449 | CACUGAACAAGUUGGCCUG | CAGGCCAACUUGUUCAGUG | | [1582-1600] |
| 450 | UGGUAGUUUCUACAGUUGG | CCAACUGUAGAAACUACCA | | [1985-2003] |
| 451 | ACCUCCCGCCAUAAAAAAC | GUUUUUUAUGGCGGGAGGU | Rb | [1380-1398] |
| 452 | GCAAUGGAUGAUUUGAUGC | GCAUCAAAUCAUCCAUUGC | | [366-384] |
| 453 | CACAGAGGAAGAGAAUCUC | GAGAUUCUCUUCCUCUGUG | | [1100-1118] |
| 454 | UAUUGAACAAUGGUUCACU | AGUGAACCAUUGUUCAAUA | | [398-416] |
| 455 | UUGGGUCUUUGAACCCUUG | CAAGGGUUCAAAGACCCAA | | [1504-1522] |
| 456 | ACUUCCAUUUGCUUUGUCC | GGACAAAGCAAAUGGAAGU | | [1555-1573] |
| 457 | AUUGAACAAUGGUUCACUG | CAGUGAACCAUUGUUCAAU | | [399-417] |
| 458 | AAGAUGUUUUGCCAACUGG | CCAGUUGGCAAAACAUCUU | | [645-663] |
| 459 | UCAUCUUCUGUCCCUUCCC | GGGAAGGGACAGAAGAUGA | | [531-549] |
| 460 | UGAAACCACCUUUUAUUAC | GUAAUAAAAGGUGGUUUCA | | [1898-1916] |
| 461 | ACCAGCUUAGAUUUUAAGG | CCUUAAAAUCUAAGCUGGU | | [1644-1662] |
| 462 | AACACUUUUCGACAUAGUG | CACUAUGUCGAAAAGUGUU | Mnk | [879-897] |
| 463 | UUUACUGUGAGGGAUGUUU | AAACAUCCCUCACAGUAAA | | [1665-1683] |
| 464 | CUACAUGUGUAACAGUUCC | GGAACUGUUACACAUGUAG | Dog, Mnk | [956-974] |
| 465 | AUUUUGGGUUUUGGGUCUU | AAGACCCAAAACCCAAAAU | | [1494-1512] |
| 466 | AUAUUUCACCCUUCAGAUC | GAUCUGAAGGGUGAAAUAU | Dog, Mnk | [1229-1247] |
| 467 | AACUUCAAGGCCCAUAUCU | AGAUAUGGGCCUUGAAGUU | | [1806-1824] |
| 468 | UUCCGAGAGCUGAAUGAGG | CCUCAUUCAGCUCUCGGAA | Rat, Mnk | [1272-1290] |
| 469 | UCUUGGUCGACCUUAGUAC | GUACUAAGGUCGACCAAGA | | [2068-2086] |
| 470 | CCACUUCACCGUACUAACC | GGUUAGUACGGUGAAGUGG | | [1755-1773] |
| 471 | AUCUGGAUCCACCAAGACU | AGUCUUGGUGGAUCCAGAU | | [2146-2164] |
| 472 | UAACUUCAAGGCCCAUAUC | GAUAUGGGCCUUGAAGUUA | | [1805-1823] |
| 473 | CUGAAGUCCAAAAAGGGUC | GACCCUUUUUGGACUUCAG | | [1356-1374] |
| 474 | ACUGUCCAGCUUUGUGCCA | UGGCACAAAGCUGGACAGU | GP | [14-32] |
| 475 | CAAAAGUCUAGAGCCACCG | CGGUGGCUCUAGACUUUUG | | [112-130] |
| 476 | UUUGGGUUUUGGGUCUUUG | CAAAGACCCAAAACCCAAA | | [1496-1514] |
| 477 | AUUUCAUCUCUUGUAUAUG | CAUAUACAAGAGAUGAAAU | | [2124-2142] |
| 478 | AACAUUUUCAGACCUAUGG | CCAUAGGUCUGAAAAUGUU | Mnk | [302-320] |
| 479 | GAAGCUGUCCCUCAUGUUG | CAACAUGAGGGACAGCUUC | | [1777-1795] |
| 480 | GUCAUCUUCUGUCCCUUCC | GGAAGGGACAGAAGAUGAC | | [530-548] |
| 481 | AAGAAACCACUGGAUGGAG | CUCCAUCCAGUGGUUUCUU | Mnk | [1209-1227] |
| 482 | ACUUUUCGACAUAGUGUGG | CCACACUAUGUCGAAAAGU | Mnk | [882-900] |
| 483 | AUCCGAGUGGAAGGAAAUU | AAUUUCCUUCCACUCGGAU | Dog, Mnk | [834-852] |
| 484 | UUUCCGUCUGGGCUUCUUG | CAAGAAGCCCAGACGGAAA | | [575-593] |
| 485 | UUGAACAAUGGUUCACUGA | UCAGUGAACCAUUGUUCAA | | [400-418] |
| 486 | UUACAAUAAAACUUUGCUG | CAGCAAAGUUUUAUUGUAA | | [2608-2626] |
| 487 | GUUCACUGAAGACCCAGGU | ACCUGGGUCUUCAGUGAAC | | [410-428] |
| 488 | GACCUUAGUACCUAAAAGG | CCUUUUAGGUACUAAGGUC | | [2076-2094] |
| 489 | CUCCCAUGUGCUCAAGACU | AGUCUUGAGCACAUGGGAG | | [70-88] |
| 490 | ACUACAUGUGUAACAGUUC | GAACUGUUACACAUGUAGU | Dog, Mnk | [955-973] |
| 491 | CCUCACCAUCAUCACACUG | CAGUGUGAUGAUGGUGAGG | Rb, Pig | [1004-1022] |
| 492 | GUAUAUGAUGAUCUGGAUC | GAUCCAGAUCAUCAUAUAC | | [2136-2154] |
| 493 | UUAGUUUACAAUCAGCCAC | GUGGCUGAUUGUAAACUAA | | [1717-1735] |
| 494 | UUUUACUGUGAGGGAUGUU | AACAUCCCUCACAGUAAAA | | [1664-1682] |
| 495 | AAAAAGGGUCAGUCUACCU | AGGUAGACUGACCCUUUUU | | [1365-1383] |
| 496 | GUUGUCAAGUCUUGCUGGC | GCCAGCAAGACUUGACAAC | | [2025-2043] |
| 497 | ACUUGUCAUGGCGACUGUC | GACAGUCGCCAUGACAAGU | | [1-19] |
| 498 | AAUAAUGUACAUCUGGCCU | AGGCCAGAUGUACAUUAUU | | [1879-1897] |
| 499 | AUUUGGAUGACAGAAACAC | GUGUUUCUGUCAUCCAAAU | Rb | [865-883] |
| 500 | AAGUCCAAAAAGGGUCAGU | ACUGACCCUUUUUGGACUU | | [1359-1377] |

TABLE B3
HTRA2 19-mer oligomer pairs - HtrA serine peptidase 2

| | | | | Human- | |
|---|---|---|---|---|---|
| | | | Other | 73747817: | Human-73747818: |
| No. | Sense | AntiSense | Sp | 603-1979 | 603-1688 |

| | | | | | |
|---|---|---|---|---|---|
| 1 | CCGAGACAGAGGGUUAAAU | AUUUAACCCUCUGUCUCGG | | [2051-2069] | [1760-1778] |
| 2 | GAAUCACAGAAACACUUUU | AAAAGUGUUUCUGUGAUUC | | [2124-2142] | [1833-1851] |
| 3 | GGCCUGGUGAUGUGAUUUU | AAAAUCACAUCACCAGGCC | | [1825-1843] | [1534-1552] |
| 4 | GUGAUGUGAUUUUGGCCAU | AUGGCCAAAAUCACAUCAC | | [1831-1849] | [1540-1558] |
| 5 | CCGUGGUCUAUAUCGAGAU | AUCUCGAUAUAGACCACGG | | [1081-1099] | [1081-1099] |
| 6 | UGACAUGGGUUUCUUGGUA | UACCAAGAAACCCAUGUCA | | [2508-2526] | [2217-2235] |
| 7 | AGGGUGAAAACUUCUGCUU | AAGCAGAAGUUUUCACCCU | | [2445-2463] | [2154-2172] |
| 8 | UGAAGAAUCACAGAAACAC | GUGUUUCUGUGAUUCUUCA | | [2120-2138] | [1829-1847] |
| 9 | UGAAGCUGUUCGAACCCAA | UUGGGUUCGAACAGCUUCA | Ms | [1886-1904] | [1595-1613] |
| 10 | GACAUGGGUUUCUUGGUAA | UUACCAAGAAACCCAUGUC | | [2509-2527] | [2218-2236] |
| 11 | CGGUUGCUGACAUGGGUUU | AAACCCAUGUCAGCAACCG | | [2501-2519] | [2210-2228] |
| 12 | GGUGAAAACUUCUGCUUGA | UCAAGCAGAAGUUUUCACC | | [2447-2465] | [2156-2174] |
| 13 | CCUAGCAACAUAUUAUAGU | ACUAUAAUAUGUUGCUAGG | | [2162-2180] | [1871-1889] |
| 14 | ACCGAGACAGAGGGUUAAA | UUUAACCCUCUGUCUCGGU | | [2050-2068] | [1759-1777] |
| 15 | GCCUGGUGAUGUGAUUUUG | CAAAAUCACAUCACCAGGC | | [1826-1844] | [1535-1553] |
| 16 | GGUAAGCUCCUGAGGUAAU | AUUACCUCAGGAGCUUACC | Rat | [2523-2541] | [2232-2250] |
| 17 | GGGUGAAAACUUCUGCUUG | CAAGCAGAAGUUUUCACCC | | [2446-2464] | [2155-2173] |
| 18 | CUGCUCUGAUUUCCUCCUU | AAGGAGGAAAUCAGAGCAG | | [2005-2023] | [1714-1732] |
| 19 | GGUGAUGUGAUUUUGGCCA | UGGCCAAAAUCACAUCACC | | [1830-1848] | [1539-1557] |
| 20 | GCCGUGGUCUAUAUCGAGA | UCUCGAUAUAGACCACGGC | | [1080-1098] | [1080-1098] |
| 21 | GUGAAAACUUCUGCUUGAC | GUCAAGCAGAAGUUUUCAC | | [2448-2466] | [2157-2175] |
| 22 | AGAUGUUUAUGAAGCUGUU | AACAGCUUCAUAAACAUCU | Ms | [1877-1895] | [1586-1604] |
| 23 | GGAGCAGAUGGUACAAAAU | AUUUUGUACCAUCUGCUCC | | [1853-1871] | [1562-1580] |
| 24 | UGAAGGUCACAGCUGGAAU | AUUCCAGCUGUGACCUUCA | | [1570-1588] | [1375-1393] |
| 25 | CGUGGUCUAUAUCGAGAUC | GAUCUCGAUAUAGACCACG | | [1082-1100] | [1082-1100] |
| 26 | GUAAGCUCCUGAGGUAAUG | CAUUACCUCAGGAGCUUAC | Rat | [2524-2542] | [2233-2251] |
| 27 | UGGGUUUCUUGGUAAGCUC | GAGCUUACCAAGAAACCCA | | [2513-2531] | [2222-2240] |
| 28 | ACAUGGGUUUCUUGGUAAG | CUUACCAAGAAACCCAUGU | | [2510-2528] | [2219-2237] |
| 29 | CCAUCUUUUGUGGGCAGUU | AACUGCCCACAAAAGAUGG | | [2311-2329] | [2020-2038] |
| 30 | CUGAUUUCCUCCUUGCCUU | AAGGCAAGGAGGAAAUCAG | | [2010-2028] | [1719-1737] |
| 31 | CACUGACCUUAUAUGUGAC | GUCACAUAUAAGGUCAGUG | | [1942-1960] | [1651-1669] |
| 32 | GCAGAUGGUACAAAAUGCU | AGCAUUUUGUACCAUCUGC | | [1856-1874] | [1565-1583] |
| 33 | CGGCCUGGUGAUGUGAUUU | AAAUCACAUCACCAGGCCG | | [1824-1842] | [1533-1551] |
| 34 | UGAAAACUUCUGCUUGACA | UGUCAAGCAGAAGUUUUCA | | [2449-2467] | [2158-2176] |
| 35 | GAGGGUGAAAACUUCUGCU | AGCAGAAGUUUUCACCCUC | | [2444-2462] | [2153-2171] |
| 36 | GGAGGGUGAAAACUUCUGC | GCAGAAGUUUUCACCCUCC | | [2443-2461] | [2152-2170] |
| 37 | ACCAUCCUGACCUCCUAUU | AAUAGGAGGUCAGGAUGGU | | [2274-2292] | [1983-2001] |
| 38 | GAAGGAGGGUGAAAACUUC | GAAGUUUUCACCCUCCUUC | | [2440-2458] | [2149-2167] |
| 39 | UGAAGGAGGGUGAAAACUU | AAGUUUUCACCCUCCUUCA | | [2439-2457] | [2148-2166] |
| 40 | CAAAAGGCUAGAGGUAAAG | CUUUACCUCUAGCCUUUUG | | [2218-2236] | [1927-1945] |
| 41 | UGCUCUGAUUUCCUCCUUG | CAAGGAGGAAAUCAGAGCA | Rat | [2006-2024] | [1715-1733] |
| 42 | UGAUGUGAUUUUGGCCAUU | AAUGGCCAAAAUCACAUCA | | [1832-1850] | [1541-1559] |
| 43 | UCCUUUGCCAUCCCUUCUG | CAGAAGGGAUGGCAAAGGA | Rat, Ms | [1590-1608] | [1395-1413] |
| 44 | CUGCCGUGGUCUAUAUCGA | UCGAUAUAGACCACGGCAG | | [1078-1096] | [1078-1096] |
| 45 | UGAGUGCGGUUGCUGACAU | AUGUCAGCAACCGCACUCA | | [2495-2513] | [2204-2222] |
| 46 | CAUCUUUUGUGGGCAGUUA | UAACUGCCCACAAAAGAUG | | [2312-2330] | [2021-2039] |
| 47 | GCUGCUGCCAUCUUUUGUG | CACAAAAGAUGGCAGCAGC | | [2304-2322] | [2013-2031] |
| 48 | UGAUUUCCUCCUUGCCUUU | AAAGGCAAGGAGGAAAUCA | | [2011-2029] | [1720-1738] |
| 49 | CCUGCUCUGAUUUCCUCCU | AGGAGGAAAUCAGAGCAGG | | [2004-2022] | [1713-1731] |
| 50 | ACAUCGCAACGCUGAGGAU | AUCCUCAGCGUUGCGAUGU | | [1285-1303] | [1285-1303] |
| 51 | UGCUCUUUGUGGUGUGGUG | CACCACACCACAAAGAGCA | | [2340-2358] | [2049-2067] |
| 52 | UGCUGCUCUUUGUGGUGUG | CACACCACAAAGAGCAGCA | | [2337-2355] | [2046-2064] |
| 53 | GUCAGGUGCUGCUCUUUGU | ACAAAGAGCAGCACCUGAC | | [2331-2349] | [2040-2058] |
| 54 | CGAGAGUUUCUGCAUCGUG | CACGAUGCAGAAACUCUCG | | [1617-1635] | [1422-1440] |
| 55 | GUGCUGCUCUUUGUGGUGU | ACACCACAAAGAGCAGCAC | | [2336-2354] | [2045-2063] |
| 56 | GGCAGUGCUGUUGUUGUUG | CAACAACAACAGCACUGCC | | [947-965] | [947-965] |
| 57 | CUCCUGCUCUGAUUUCCUC | GAGGAAAUCAGAGCAGGAG | | [2002-2020] | [1711-1729] |
| 58 | CAAAAUGCUGAAGAUGUUU | AAACAUCUUCAGCAUUUUG | Rat, Ms | [1866-1884] | [1575-1593] |
| 59 | GAUGGUACAAAAUGCUGAA | UUCAGCAUUUUGUACCAUC | | [1859-1877] | [1568-1586] |
| 60 | AGAUGGUACAAAAUGCUGA | UCAGCAUUUUGUACCAUCU | | [1858-1876] | [1567-1585] |
| 61 | AGACCACAAUUCCCGGCAU | AUGCCGGGAAUUGUGGUCU | | [423-441] | [423-441] |
| 62 | CCCGGAGUCAGUACAACUU | AAGUUGUACUGACUCCGGG | | [1030-1048] | [1030-1048] |
| 63 | AGAAUGAAUAGAUCACCAA | UUGGUGAUCUAUUCAUUCU | | [1973-1991] | [1682-1700] |
| 64 | GGACGAGAAACACUGACCU | AGGUCAGUGUUUCUCGUCC | | [1932-1950] | [1641-1659] |
| 65 | GCCUCAGAGAACUCUGGAA | UUCCAGAGUUCUCUGAGGC | | [885-903] | [885-903] |
| 66 | CUGCUGACGUCAGGAACUU | AAGUUCCUGACGUCAGCAG | | [714-732] | [714-732] |
| 67 | UGAACACCAUGAAGGUCAC | GUGACCUUCAUGGUGUUCA | | [1561-1579] | [1366-1384] |
| 68 | CUCUGAAGAAUCACAGAAA | UUUCUGUGAUUCUUCAGAG | | [2117-2135] | [1826-1844] |
| 69 | CAGUGCUGUUGUUGUUGUG | CACAACAACAACAGCACUG | | [949-967] | [949-967] |
| 70 | CGCUGAGGAUUCAGACUAA | UUAGUCUGAAUCCUCAGCG | | [1294-1312] | [1294-1312] |
| 71 | ACGCUGAGGAUUCAGACUA | UAGUCUGAAUCCUCAGCGU | | [1293-1311] | [1293-1311] |
| 72 | UUGCUGACAUGGGUUUCUU | AAGAAACCCAUGUCAGCAA | | [2504-2522] | [2213-2231] |
| 73 | GCUCUGAAGAAUCACAGAA | UUCUGUGAUUCUUCAGAGC | | [2116-2134] | [1825-1843] |
| 74 | CCAAGAGUAUGAGGCUCCU | AGGAGCCUCAUACUCUUGG | | [1988-2006] | [1697-1715] |
| 75 | AGGUCACAGAAUGAAUAGA | UCUAUUCAUUCUGUGACCU | | [1966-1984] | [1675-1693] |
| 76 | UGGUACAAAAUGCUGAAGA | UCUUCAGCAUUUUGUACCA | | [1861-1879] | [1570-1588] |
| 77 | UGAUGAUGCUGACCCUGAG | CUCAGGGUCAGCAUCAUCA | | [1693-1711] | [1498-1516] |
| 78 | GUGCACUUCUGAAGGACUU | AAGUCCUUCAGAAGUGCAC | | [487-505] | [487-505] |
| 79 | GGGAAAAGAAGAAUUCCUC | GAGGAAUUCUUCUUUUCCC | | [1636-1654] | [1441-1459] |
| 80 | UCCCAUGCUUGGCUACAGA | UCUGUAGCCAAGCAUGGGA | | [2403-2421] | [2112-2130] |
| 81 | CCGGAGUCAGUACAACUUC | GAAGUUGUACUGACUCCGG | | [1031-1049] | [1031-1049] |
| 82 | AGGGUUAAAUGAACCAGUG | CACUGGUUCAUUUAACCCU | | [2060-2078] | [1769-1787] |
| 83 | GAGGGUUAAAUGAACCAGU | ACUGGUUCAUUUAACCCUC | | [2059-2077] | [1768-1786] |
| 84 | CACCGAGACAGAGGGUUAA | UUAACCCUCUGUCUCGGUG | | [2049-2067] | [1758-1776] |
| 85 | CCUCAGAGAACUCUGGAAC | GUUCCAGAGUUCUCUGAGG | | [886-904] | [886-904] |
| 86 | UGAUUGGAGUGAACACCAU | AUGGUGUUCACUCCAAUCA | Rat, Ms | [1552-1570] | [1357-1375] |
| 87 | UUGGUAAGCUCCUGAGGUA | UACCUCAGGAGCUUACCAA | | [2521-2539] | [2230-2248] |
| 88 | CCAUAGUGCAUGGUCUGAU | AUCAGACCAUGCACUAUGG | | [2477-2495] | [2186-2204] |
| 89 | GGGCUCUGAAGAAUCACAG | CUGUGAUUCUUCAGAGCCC | | [2114-2132] | [1823-1841] |
| 90 | CUCCUGGGCUCUGAAGAAU | AUUCUUCAGAGCCCAGGAG | | [2109-2127] | [1818-1836] |
| 91 | AGAGGGUUAAAUGAACCAG | CUGGUUCAUUUAACCCUCU | | [2058-2076] | [1767-1785] |
| 92 | GAAGAUGUUUAUGAAGCUG | CAGCUUCAUAAACAUCUUC | Ms | [1875-1893] | [1584-1602] |
| 93 | GAAAAGAAGAAUUCCUCCU | AGGAGGAAUUCUUCUUUUC | | [1638-1656] | [1443-1461] |
| 94 | UGAUCGUCUUCGAGAGUUU | AAACUCUCGAAGACGAUCA | | [1607-1625] | [1412-1430] |
| 95 | CUGCUUGACAGUUCCACAU | AUGUGGAACUGUCAAGCAG | | [2458-2476] | [2167-2185] |
| 96 | UGAGCUGCUGCCAUCUUUU | AAAAGAUGGCAGCAGCUCA | | [2301-2319] | [2010-2028] |
| 97 | CCACCAAAAGGCUAGAGGU | ACCUCUAGCCUUUUGGUGG | | [2214-2232] | [1923-1941] |
| 98 | AGAUCACCAAGAGUAUGAG | CUCAUACUCUUGGUGAUCU | | [1982-2000] | [1691-1709] |
| 99 | CUGAUCGUCUUCGAGAGUU | AACUCUCGAAGACGAUCAG | | [1606-1624] | [1411-1429] |
| 100 | CCCUUCUGAUCGUCUUCGA | UCGAAGACGAUCAGAAGGG | | [1601-1619] | [1406-1424] |
| 101 | UUGGAGUGAACACCAUGAA | UUCAUGGUGUUCACUCCAA | Rat, Ms | [1555-1573] | [1360-1378] |
| 102 | GUCCGUGUGAGACUGCUAA | UUAGCAGUCUCACACGGAC | | [1218-1236] | [1218-1236] |
| 103 | ACAACUUCAUCGCAGAUGU | ACAUCUGCGAUGAAGUUGU | | [1042-1060] | [1042-1060] |
| 104 | CAGUACAACUUCAUCGCAG | CUGCGAUGAAGUUGUACUG | | [1038-1056] | [1038-1056] |
| 105 | CUCUGAAGGAGGGUGAAAA | UUUUCACCCUCCUUCAGAG | | [2436-2454] | [2145-2163] |
| 106 | CCAUGCUUGGCUACAGAUA | UAUCUGUAGCCAAGCAUGG | | [2405-2423] | [2114-2132] |
| 107 | AGCAAAGAUUCCCAUGCUU | AAGCAUGGGAAUCUUUGCU | | [2394-2412] | [2103-2121] |
| 108 | UGACCUCCUAUUAAAGAAA | UUUCUUUAAUAGGAGGUCA | | [2281-2299] | [1990-2008] |
| 109 | UCUGAAGAAUCACAGAAAC | GUUUCUGUGAUUCUUCAGA | | [2118-2136] | [1827-1845] |
| 110 | UCCUUGCCUUUCUGGCUGA | UCAGCCAGAAAGGCAAGGA | | [2019-2037] | [1728-1746] |
| 111 | CAGAAUGAAUAGAUCACCA | UGGUGAUCUAUUCAUUCUG | | [1972-1990] | [1681-1699] |
| 112 | GCUGACGUCAGGAACUUCU | AGAAGUUCCUGACGUCAGC | | [716-734] | [716-734] |
| 113 | GCUGAAGAUGUUUAUGAAG | CUUCAUAAACAUCUUCAGC | Ms | [1872-1890] | [1581-1599] |
| 114 | GGAAGCUCCAUAACUGCUG | CAGCAGUUAUGGAGCUUCC | | [215-233] | [215-233] |
| 115 | UGGUCUAUAUCGAGAUCCU | AGGAUCUCGAUAUAGACCA | Rat, Ms | [1084-1102] | [1084-1102] |
| 116 | GAAAACUUCUGCUUGACAG | CUGUCAAGCAGAAGUUUUC | | [2450-2468] | [2159-2177] |
| 117 | CCCAUGCUUGGCUACAGAU | AUCUGUAGCCAAGCAUGGG | | [2404-2422] | [2113-2131] |
| 118 | GGCUCUGAAGAAUCACAGA | UCUGUGAUUCUUCAGAGCC | | [2115-2133] | [1824-1842] |
| 119 | AGAGAACUCUGGAACCCGU | ACGGGUUCCAGAGUUCUCU | | [890-908] | [890-908] |
| 120 | GCACUUCUGAAGGACUUCA | UGAAGUCCUUCAGAAGUGC | | [489-507] | [489-507] |
| 121 | CCUCUGAAGGAGGGUGAAA | UUUCACCCUCCUUCAGAGG | | [2435-2453] | [2144-2162] |
| 122 | GAAAAUGAGCUGCUGCCAU | AUGGCAGCAGCUCAUUUUC | | [2296-2314] | [2005-2023] |
| 123 | CCUCCUAUUAAAGAAAAUG | CAUUUUCUUUAAUAGGAGG | | [2284-2302] | [1993-2011] |
| 124 | UGGGCUCUGAAGAAUCACA | UGUGAUUCUUCAGAGCCCA | | [2113-2131] | [1822-1840] |
| 125 | CAGAGGGUUAAAUGAACCA | UGGUUCAUUUAACCCUCUG | | [2057-2075] | [1766-1784] |
| 126 | AGAAUUCCUCCUCCGGAAU | AUUCCGGAGGAGGAAUUCU | | [1645-1663] | [1450-1468] |
| 127 | AAGAAUUCCUCCUCCGGAA | UUCCGGAGGAGGAAUUCUU | | [1644-1662] | [1449-1467] |
| 128 | CUGGAAUCUCCUUUGCCAU | AUGGCAAAGGAGAUUCCAG | Rat, Ms | [1582-1600] | [1387-1405] |
| 129 | CCACAUCCAUAGUGCAUGG | CCAUGCACUAUGGAUGUGG | | [2471-2489] | [2180-2198] |
| 130 | GCAAAGAUUCCCAUGCUUG | CAAGCAUGGGAAUCUUUGC | | [2395-2413] | [2104-2122] |
| 131 | GAGCAAAGAUUCCCAUGCU | AGCAUGGGAAUCUUUGCUC | | [2393-2411] | [2102-2120] |
| 132 | GCCAUCUUUUGUGGGCAGU | ACUGCCCACAAAAGAUGGC | | [2310-2328] | [2019-2037] |
| 133 | CUGACCUCCUAUUAAAGAA | UUCUUUAAUAGGAGGUCAG | | [2280-2298] | [1989-2007] |
| 134 | CACAGAAUGAAUAGAUCAC | GUGAUCUAUUCAUUCUGUG | | [1970-1988] | [1679-1697] |
| 135 | CCCUGAGGUCACAGAAUGA | UCAUUCUGUGACCUCAGGG | | [1961-1979] | [1670-1688] |
| 136 | UGCCAUCCCUUCUGAUCGU | ACGAUCAGAAGGGAUGGCA | | [1595-1613] | [1400-1418] |
| 137 | GGCUACAGAUACUGACAGC | GCUGUCAGUAUCUGUAGCC | | [2413-2431] | [2122-2140] |
| 138 | UGGCUACAGAUACUGACAG | CUGUCAGUAUCUGUAGCCA | | [2412-2430] | [2121-2139] |
| 139 | GGAGCAAAGAUUCCCAUGC | GCAUGGGAAUCUUUGCUCC | | [2392-2410] | [2101-2119] |
| 140 | AGAUACUGGAGCUGACCAU | AUGGUCAGCUCCAGUAUCU | | [2260-2278] | [1969-1987] |
| 141 | AGCUCCAUAACUGCUGCUU | AAGCAGCAGUUAUGGAGCU | | [218-236] | [218-236] |
| 142 | GCUGAGGAUUCAGACUAAG | CUUAGUCUGAAUCCUCAGC | | [1295-1313] | [1295-1313] |
| 143 | CAGUUCCACAUCCAUAGUG | CACUAUGGAUGUGGAACUG | | [2466-2484] | [2175-2193] |
| 144 | UGACAGUUCCACAUCCAUA | UAUGGAUGUGGAACUGUCA | | [2463-2481] | [2172-2190] |
| 145 | UGGGAGCAAAGAUUCCCAU | AUGGGAAUCUUUGCUCCCA | | [2390-2408] | [2099-2117] |
| 146 | UGCCAUCUUUUGUGGGCAG | CUGCCCACAAAAGAUGGCA | | [2309-2327] | [2018-2036] |
| 147 | AUGAGCUGCUGCCAUCUUU | AAAGAUGGCAGCAGCUCAU | | [2300-2318] | [2009-2027] |
| 148 | GCUAGAGGUAAAGCUGUAU | AUACAGCUUUACCUCUAGC | | [2224-2242] | [1933-1951] |
| 149 | CACCAAAAGGCUAGAGGUA | UACCUCUAGCCUUUUGGUG | | [2215-2233] | [1924-1942] |
| 150 | CACUUCUGAAGGACUUCAG | CUGAAGUCCUUCAGAAGUG | | [490-508] | [490-508] |
| 151 | CAUCCCUUCUGAUCGUCUU | AAGACGAUCAGAAGGGAUG | | [1598-1616] | [1403-1421] |
| 152 | CCAUCCCUUCUGAUCGUCU | AGACGAUCAGAAGGGAUGG | | [1597-1615] | [1402-1420] |
| 153 | GGAAUCUCCUUUGCCAUCC | GGAUGGCAAAGGAGAUUCC | Rat, Ms | [1584-1602] | [1389-1407] |
| 154 | GUGAACACCAUGAAGGUCA | UGACCUUCAUGGUGUUCAC | | [1560-1578] | [1365-1383] |
| 155 | UGGGCUCAUUGUCACCAAC | GUUGGUGACAAUGAGCCCA | | [1172-1190] | [1172-1190] |
| 156 | AGAUUCCCAUGCUUGGCUA | UAGCCAAGCAUGGGAAUCU | | [2399-2417] | [2108-2126] |
| 157 | CUGCCAUCUUUUGUGGGCA | UGCCCACAAAAGAUGGCAG | | [2308-2326] | [2017-2035] |
| 158 | ACCAAAAGGCUAGAGGUAA | UUACCUCUAGCCUUUUGGU | | [2216-2234] | [1925-1943] |
| 159 | UGCCUUUCUGGCUGAGGUU | AACCUCAGCCAGAAAGGCA | | [2023-2041] | [1732-1750] |
| 160 | GCUCUGAUUUCCUCCUUGC | GCAAGGAGGAAAUCAGAGC | Rat | [2007-2025] | [1716-1734] |
| 161 | AUGGGCUCAUUGUCACCAA | UUGGUGACAAUGAGCCCAU | | [1171-1189] | [1171-1189] |
| 162 | UCAUCGCAGAUGUGGUGGA | UCCACCACAUCUGCGAUGA | Ms | [1048-1066] | [1048-1066] |
| 163 | GACAGUUCCACAUCCAUAG | CUAUGGAUGUGGAACUGUC | | [2464-2482] | [2173-2191] |
| 164 | GCUACAGAUACUGACAGCU | AGCUGUCAGUAUCUGUAGC | | [2414-2432] | [2123-2141] |
| 165 | CAGUUAGUCAGGUGCUGCU | AGCAGCACCUGACUAACUG | | [2325-2343] | [2034-2052] |
| 166 | GAGGCUCCUGCUCUGAUUU | AAAUCAGAGCAGGAGCCUC | | [1998-2016] | [1707-1725] |
| 167 | ACAGCUGGAAUCUCCUUUG | CAAAGGAGAUUCCAGCUGU | Rat, Ms | [1578-1596] | [1383-1401] |
| 168 | CACAGCUGGAAUCUCCUUU | AAAGGAGAUUCCAGCUGUG | | [1577-1595] | [1382-1400] |
| 169 | GGGAGCAAAGAUUCCCAUG | CAUGGGAAUCUUUGCUCCC | | [2391-2409] | [2100-2118] |
| 170 | GACCAUCCUGACCUCCUAU | AUAGGAGGUCAGGAUGGUC | | [2273-2291] | [1982-2000] |
| 171 | UCGAGAGUUUCUGCAUCGU | ACGAUGCAGAAACUCUCGA | | [1616-1634] | [1421-1439] |
| 172 | GGAGUGAACACCAUGAAGG | CCUUCAUGGUGUUCACUCC | Rat, Ms | [1557-1575] | [1362-1380] |
| 173 | CCUGAGGUCACAGAAUGAA | UUCAUUCUGUGACCUCAGG | | [1962-1980] | [1671-1689] |
| 174 | UCACAGCUGGAAUCUCCUU | AAGGAGAUUCCAGCUGUGA | | [1576-1594] | [1381-1399] |
| 175 | GCUGGAAUCUCCUUUGCCA | UGGCAAAGGAGAUUCCAGC | Rat, Ms | [1581-1599] | [1386-1404] |
| 176 | GCAACGCUGAGGAUUCAGA | UCUGAAUCCUCAGCGUUGC | | [1290-1308] | [1290-1308] |
| 177 | CUGAGGUCACAGAAUGAAU | AUUCAUUCUGUGACCUCAG | | [1963-1981] | [1672-1690] |
| 178 | CACAUCCAUAGUGCAUGGU | ACCAUGCACUAUGGAUGUG | | [2472-2490] | [2181-2199] |
| 179 | CAGCUAUUGAUUUUGGAAA | UUUCCAAAAUCAAUAGCUG | | | |
| 180 | GCUAUUGAUUUUGGAAACU | AGUUUCCAAAAUCAAUAGC | | | |
| 181 | AGCUAUUGAUUUUGGAAAC | GUUUCCAAAAUCAAUAGCU | | | |

TABLE B4
HTRA2 - 19-mer oligomer pairs HtrA serine peptidase 2

| | | | | Human- | Human- |
|---|---|---|---|---|---|
| | | | | 73747817: | 73747818: |
| No. | Sense | AntiSense | Other Sp | 603-1979 | 603-1688 |

| | | | | | |
|---|---|---|---|---|---|
| 1 | GCAACAUAUUAUAGUAAAA | UUUUACUAUAAUAUGUUGC | | [2166-2184] | [1875-1893] |
| 2 | CCAAAAGGCUAGAGGUAAA | UUUACCUCUAGCCUUUUGG | | [2217-2235] | [1926-1944] |
| 3 | GGGAGCAGAUGGUACAAAA | UUUUGUACCAUCUGCUCCC | | [1852-1870] | [1561-1579] |
| 4 | GAGAAACACUGACCUUAUA | UAUAAGGUCAGUGUUUCUC | | [1936-1954] | [1645-1663] |
| 5 | GGGAGGUGAUUGGAGUGAA | UUCACUCCAAUCACCUCCC | Rat, Ms, GP | [1546-1564] | [1351-1369] |
| 6 | CGAGAAACACUGACCUUAU | AUAAGGUCAGUGUUUCUCG | | [1935-1953] | [1644-1662] |
| 7 | GAAGCUGUUCGAACCCAAU | AUUGGGUUCGAACAGCUUC | Ms | [1887-1905] | [1596-1614] |
| 8 | CCUGAGGGAAGCUCCAUAA | UUAUGGAGCUUCCCUCAGG | | [209-227] | [209-227] |
| 9 | UGCUGAAGAUGUUUAUGAA | UUCAUAAACAUCUUCAGCA | Ms | [1871-1889] | [1580-1598] |
| 10 | GAGACAGAGGGUUAAAUGA | UCAUUUAACCCUCUGUCUC | | [2053-2071] | [1762-1780] |
| 11 | CUUACCGGUGCGAGUCAAA | UUUGACUCGCACCGGUAAG | | [171-189] | [171-189] |
| 12 | GGAGGUGAUUGGAGUGAAC | GUUCACUCCAAUCACCUCC | Rat, Ms, GP | [1547-1565] | [1352-1370] |
| 13 | GAAUAGAUCACCAAGAGUA | UACUCUUGGUGAUCUAUUC | | [1978-1996] | [1687-1705] |
| 14 | AAUAGAUCACCAAGAGUAU | AUACUCUUGGUGAUCUAUU | | [1979-1997] | [1688-1706] |
| 15 | GGUACAAAAUGCUGAAGAU | AUCUUCAGCAUUUUGUACC | | [1862-1880] | [1571-1589] |
| 16 | GCUUGGCUACAGAUACUGA | UCAGUAUCUGUAGCCAAGC | | [2409-2427] | [2118-2136] |
| 17 | AGACAGAGGGUUAAAUGAA | UUCAUUUAACCCUCUGUCU | | [2054-2072] | [1763-1781] |
| 18 | CAGAAACACUUUUUAUAUA | UAUAUAAAAAGUGUUUCUG | | [2130-2148] | [1839-1857] |
| 19 | AAUUAUACCUAGCAACAUA | UAUGUUGCUAGGUAUAAUU | | [2155-2173] | [1864-1882] |
| 20 | AGCAACAUAUUAUAGUAAA | UUUACUAUAAUAUGUUGCU | | [2165-2183] | [1874-1892] |
| 21 | CUAGCAACAUAUUAUAGUA | UACUAUAAUAUGUUGCUAG | | [2163-2181] | [1872-1890] |
| 22 | CACAGAAACACUUUUUAUA | UAUAAAAAGUGUUUCUGUG | | [2128-2146] | [1837-1855] |
| 23 | UGAUGAGUGCGGUUGCUGA | UCAGCAACCGCACUCAUCA | | [2492-2510] | [2201-2219] |
| 24 | CAUCCUGACCUCCUAUUAA | UUAAUAGGAGGUCAGGAUG | | [2276-2294] | [1985-2003] |
| 25 | CGGAGUCAGUACAACUUCA | UGAAGUUGUACUGACUCCG | | [1032-1050] | [1032-1050] |
| 26 | GAAGAAUCACAGAAACACU | AGUGUUUCUGUGAUUCUUC | | [2121-2139] | [1830-1848] |
| 27 | AGUACAACUUCAUCGCAGA | UCUGCGAUGAAGUUGUACU | | [1039-1057] | [1039-1057] |
| 28 | UGAGGUCACAGAAUGAAUA | UAUUCAUUCUGUGACCUCA | | [1964-1982] | [1673-1691] |
| 29 | AUAAAAUUAUACCUAGCAA | UUGCUAGGUAUAAUUUUAU | Rat, Ms | [2151-2169] | [1860-1878] |
| 30 | CAUGAAGGUCACAGCUGGA | UCCAGCUGUGACCUUCAUG | | [1568-1586] | [1373-1391] |
| 31 | UACCUAGCAACAUAUUAUA | UAUAAUAUGUUGCUAGGUA | | [2160-2178] | [1869-1887] |
| 32 | GGCUAGAGGUAAAGCUGUA | UACAGCUUUACCUCUAGCC | | [2223-2241] | [1932-1950] |
| 33 | CCUGGGCUCUGAAGAAUCA | UGAUUCUUCAGAGCCCAGG | | [2111-2129] | [1820-1838] |
| 34 | CCUUUGCCAUCCCUUCUGA | UCAGAAGGGAUGGCAAAGG | Rat, Ms | [1591-1609] | [1396-1414] |
| 35 | UCGCAGAUGUGGUGGAGAA | UUCUCCACCACAUCUGCGA | Ms | [1051-1069] | [1051-1069] |
| 36 | GUUCGAACCCAAUCCCAGU | ACUGGGAUUGGGUUCGAAC | | [1893-1911] | [1602-1620] |
| 37 | GACAGAGGGUUAAAUGAAC | GUUCAUUUAACCCUCUGUC | | [2055-2073] | [1764-1782] |
| 38 | AGAUGUGGUGGAGAAGACA | UGUCUUCUCCACCACAUCU | Ms | [1055-1073] | [1055-1073] |
| 39 | CAUAGUGCAUGGUCUGAUG | CAUCAGACCAUGCACUAUG | | [2478-2496] | [2187-2205] |
| 40 | AUCCUGACCUCCUAUUAAA | UUUAAUAGGAGGUCAGGAU | | [2277-2295] | [1986-2004] |
| 41 | CCAUCCUGACCUCCUAUUA | UAAUAGGAGGUCAGGAUGG | | [2275-2293] | [1984-2002] |
| 42 | GGUCACAGAAUGAAUAGAU | AUCUAUUCAUUCUGUGACC | | [1967-1985] | [1676-1694] |
| 43 | GAAUGAAUAGAUCACCAAG | CUUGGUGAUCUAUUCAUUC | | [1974-1992] | [1683-1701] |
| 44 | UACCGGUGCGAGUCAAAGA | UCUUUGACUCGCACCGGUA | | [173-191] | [173-191] |
| 45 | CGAGACAGAGGGUUAAAUG | CAUUUAACCCUCUGUCUCG | | [2052-2070] | [1761-1779] |
| 46 | ACAAAAUGCUGAAGAUGUU | AACAUCUUCAGCAUUUUGU | Rat, Ms | [1865-1883] | [1574-1592] |
| 47 | GGGCAGUGCUGUUGUUGUU | AACAACAACAGCACUGCCC | | [946-964] | [946-964] |
| 48 | CUCCAUAACUGCUGCUUCA | UGAAGCAGCAGUUAUGGAG | | [220-238] | [220-238] |
| 49 | UGUUUAUGAAGCUGUUCGA | UCGAACAGCUUCAUAAACA | Ms | [1880-1898] | [1589-1607] |
| 50 | GUUUAUGAAGCUGUUCGAA | UUCGAACAGCUUCAUAAAC | Ms | [1881-1899] | [1590-1608] |
| 51 | GAGGGAAGCUCCAUAACUG | CAGUUAUGGAGCUUCCCUC | | [212-230] | [212-230] |
| 52 | AGUAAAAAAUGAGGUGGGA | UCCCACCUCAUUUUUUACU | | [2178-2196] | [1887-1905] |
| 53 | ACUCCUGGGCUCUGAAGAA | UUCUUCAGAGCCCAGGAGU | | [2108-2126] | [1817-1835] |
| 54 | GCAGAUGUGGUGGAGAAGA | UCUUCUCCACCACAUCUGC | Ms, GP | [1053-1071] | [1053-1071] |
| 55 | AAAUGCUGAAGAUGUUUAU | AUAAACAUCUUCAGCAUUU | Rat, Ms | [1868-1886] | [1577-1595] |
| 56 | CAUAACUGCUGCUUCAGGA | UCCUGAAGCAGCAGUUAUG | | [223-241] | [223-241] |
| 57 | UGACCAUCCUGACCUCCUA | UAGGAGGUCAGGAUGGUCA | | [2272-2290] | [1981-1999] |
| 58 | ACACUGACCUUAUAUGUGA | UCACAUAUAAGGUCAGUGU | | [1941-1959] | [1650-1668] |
| 59 | ACGAGAAACACUGACCUUA | UAAGGUCAGUGUUUCUCGU | | [1934-1952] | [1643-1661] |
| 60 | CAUCCAUAGUGCAUGGUCU | AGACCAUGCACUAUGGAUG | | [2474-2492] | [2183-2201] |
| 61 | GACCUCCUAUUAAAGAAAA | UUUUCUUUAAUAGGAGGUC | | [2282-2300] | [1991-2009] |
| 62 | GGAGUCAGUACAACUUCAU | AUGAAGUUGUACUGACUCC | | [1033-1051] | [1033-1051] |
| 63 | AAAAUUAUACCUAGCAACA | UGUUGCUAGGUAUAAUUUU | Rat | [2153-2171] | [1862-1880] |
| 64 | UGAAUAGAUCACCAAGAGU | ACUCUUGGUGAUCUAUUCA | | [1977-1995] | [1686-1704] |
| 65 | UGGUCUGAUGAGUGCGGUU | AACCGCACUCAUCAGACCA | | [2487-2505] | [2196-2214] |
| 66 | GACGAGAAACACUGACCUU | AAGGUCAGUGUUUCUCGUC | | [1933-1951] | [1642-1660] |
| 67 | GAGUGAACACCAUGAAGGU | ACCUUCAUGGUGUUCACUC | Rat, Ms | [1558-1576] | [1363-1381] |
| 68 | UAUACCUAGCAACAUAUUA | UAAUAUGUUGCUAGGUAUA | | [2158-2176] | [1867-1885] |
| 69 | GAGGUGAUUGGAGUGAACA | UGUUCACUCCAAUCACCUC | Rat, Ms, GP | [1548-1566] | [1353-1371] |
| 70 | UGGGAGGGCUGGAUCUUUU | AAAAGAUCCAGCCCUCCCA | | [2192-2210] | [1901-1919] |
| 71 | AGAAACACUGACCUUAUAU | AUAUAAGGUCAGUGUUUCU | | [1937-1955] | [1646-1664] |
| 72 | ACAGAAACACUUUUUAUAU | AUAUAAAAAGUGUUUCUGU | | [2129-2147] | [1838-1856] |
| 73 | CUGUUCGAACCCAAUCCCA | UGGGAUUGGGUUCGAACAG | | [1891-1909] | [1600-1618] |
| 74 | GUUCCACAUCCAUAGUGCA | UGCACUAUGGAUGUGGAAC | | [2468-2486] | [2177-2195] |
| 75 | GAAGAAUUCCUCCUCCGGA | UCCGGAGGAGGAAUUCUUC | | [1643-1661] | [1448-1466] |
| 76 | AUACCUAGCAACAUAUUAU | AUAAUAUGUUGCUAGGUAU | | [2159-2177] | [1868-1886] |
| 77 | AAUGAAUAGAUCACCAAGA | UCUUGGUGAUCUAUUCAUU | | [1975-1993] | [1684-1702] |
| 78 | GUGAUGAUGCUGACCCUGA | UCAGGGUCAGCAUCAUCAC | Ms | [1692-1710] | [1497-1515] |
| 79 | UCACAGAAACACUUUUUAU | AUAAAAAGUGUUUCUGUGA | | [2127-2145] | [1836-1854] |
| 80 | AGUGCAUGGUCUGAUGAGU | ACUCAUCAGACCAUGCACU | | [2481-2499] | [2190-2208] |
| 81 | GAUGAUGCUGACCCUGAGU | ACUCAGGGUCAGCAUCAUC | | [1694-1712] | [1499-1517] |
| 82 | CUUUGCCAUCCCUUCUGAU | AUCAGAAGGGAUGGCAAAG | Rat, Ms | [1592-1610] | [1397-1415] |
| 83 | UCUAUAUCGAGAUCCUGGA | UCCAGGAUCUCGAUAUAGA | | [1087-1105] | [1087-1105] |
| 84 | UAGCAACAUAUUAUAGUAA | UUACUAUAAUAUGUUGCUA | | [2164-2182] | [1873-1891] |
| 85 | CUUGACAGUUCCACAUCCA | UGGAUGUGGAACUGUCAAG | | [2461-2479] | [2170-2188] |
| 86 | GAGAACUCUGGAACCCGUU | AACGGGUUCCAGAGUUCUC | | [891-909] | [891-909] |
| 87 | CUUCUGAUCGUCUUCGAGA | UCUCGAAGACGAUCAGAAG | | [1603-1621] | [1408-1426] |
| 88 | AGGUGAUUGGAGUGAACAC | GUGUUCACUCCAAUCACCU | Rat, Ms, GP | [1549-1567] | [1354-1372] |
| 89 | CAUUGUCACCAACGCCCAU | AUGGGCGUUGGUGACAAUG | | [1178-1196] | [1178-1196] |
| 90 | GAGAGUUUCUGCAUCGUGG | CCACGAUGCAGAAACUCUC | | [1618-1636] | [1423-1441] |
| 91 | GAGCAGUAGGAAGCAGUCA | UGACUGCUUCCUACUGCUC | | [68-86] | [68-86] |
| 92 | UCUGCUUGACAGUUCCACA | UGUGGAACUGUCAAGCAGA | | [2457-2475] | [2166-2184] |
| 93 | GAGGUCACAGAAUGAAUAG | CUAUUCAUUCUGUGACCUC | | [1965-1983] | [1674-1692] |
| 94 | CAUGCUUGGCUACAGAUAC | GUAUCUGUAGCCAAGCAUG | | [2406-2424] | [2115-2133] |
| 95 | UGAAGAUGUUUAUGAAGCU | AGCUUCAUAAACAUCUUCA | Ms | [1874-1892] | [1583-1601] |
| 96 | AGCUCCUGAGGUAAUGGCA | UGCCAUUACCUCAGGAGCU | Rat | [2527-2545] | [2236-2254] |
| 97 | UUUUGUGGGCAGUUAGUCA | UGACUAACUGCCCACAAAA | | [2316-2334] | [2025-2043] |
| 98 | GUGAUUGGAGUGAACACCA | UGGUGUUCACUCCAAUCAC | Rat, Ms | [1551-1569] | [1356-1374] |
| 99 | UGGUAAGCUCCUGAGGUAA | UUACCUCAGGAGCUUACCA | Rat | [2522-2540] | [2231-2249] |
| 100 | CAGAUGUGGUGGAGAAGAC | GUCUUCUCCACCACAUCUG | Ms, GP | [1054-1072] | [1054-1072] |
| 101 | UUGGGAAGGCGGAGUCUUU | AAAGACUCCGCCUUCCCAA | | [293-311] | [293-311] |
| 102 | AAAUUAUACCUAGCAACAU | AUGUUGCUAGGUAUAAUUU | Rat | [2154-2172] | [1863-1881] |
| 103 | AUUGGAGUGAACACCAUGA | UCAUGGUGUUCACUCCAAU | Rat, Ms | [1554-1572] | [1359-1377] |
| 104 | GAUUGGAGUGAACACCAUG | CAUGGUGUUCACUCCAAUC | Rat, Ms | [1553-1571] | [1358-1376] |
| 105 | AUCACAGAAACACUUUUUA | UAAAAAGUGUUUCUGUGAU | | [2126-2144] | [1835-1853] |
| 106 | GAAACACUGACCUUAUAUG | CAUAUAAGGUCAGUGUUUC | | [1938-1956] | [1647-1665] |
| 107 | UGUUCGAACCCAAUCCCAG | CUGGGAUUGGGUUCGAACA | | [1892-1910] | [1601-1619] |
| 108 | UCUUACCGGUGCGAGUCAA | UUGACUCGCACCGGUAAGA | | [170-188] | [170-188] |
| 109 | AUUAUACCUAGCAACAUAU | AUAUGUUGCUAGGUAUAAU | | [2156-2174] | [1865-1883] |
| 110 | CACCUGCCGUGGUCUAUAU | AUAUAGACCACGGCAGGUG | | [1075-1093] | [1075-1093] |
| 111 | UCUGAAGGAGGGUGAAAAC | GUUUUCACCCUCCUUCAGA | | [2437-2455] | [2146-2164] |
| 112 | UUGGCUACAGAUACUGACA | UGUCAGUAUCUGUAGCCAA | | [2411-2429] | [2120-2138] |
| 113 | GAAGGUCACAGCUGGAAUC | GAUUCCAGCUGUGACCUUC | GP | [1571-1589] | [1376-1394] |
| 114 | CUGAAGAAUCACAGAAACA | UGUUUCUGUGAUUCUUCAG | | [2119-2137] | [1828-1846] |
| 115 | UCGCAACGCUGAGGAUUCA | UGAAUCCUCAGCGUUGCGA | | [1288-1306] | [1288-1306] |
| 116 | AAGAAUCACAGAAACACUU | AAGUGUUUCUGUGAUUCUU | | [2122-2140] | [1831-1849] |
| 117 | GUCACAGAAUGAAUAGAUC | GAUCUAUUCAUUCUGUGAC | | [1968-1986] | [1677-1695] |
| 118 | UCUUUUGUGGGCAGUUAGU | ACUAACUGCCCACAAAAGA | | [2314-2332] | [2023-2041] |
| 119 | UUGACAGUUCCACAUCCAU | AUGGAUGUGGAACUGUCAA | | [2462-2480] | [2171-2189] |
| 120 | UGACGUCAGGAACUUCUGA | UCAGAAGUUCCUGACGUCA | | [718-736] | [718-736] |
| 121 | CUUCUGCUUGACAGUUCCA | UGGAACUGUCAAGCAGAAG | | [2455-2473] | [2164-2182] |
| 122 | CUUGGCUACAGAUACUGAC | GUCAGUAUCUGUAGCCAAG | | [2410-2428] | [2119-2137] |
| 123 | UAAGCGGCGACACGUAUGA | UCAUACGUGUCGCCGCUUA | | [1234-1252] | [1234-1252] |
| 124 | AUGAAGGUCACAGCUGGAA | UUCCAGCUGUGACCUUCAU | GP | [1569-1587] | [1374-1392] |
| 125 | UGGAGUGAACACCAUGAAG | CUUCAUGGUGUUCACUCCA | Rat, Ms | [1556-1574] | [1361-1379] |
| 126 | AGUCCGUGUGAGACUGCUA | UAGCAGUCUCACACGGACU | | [1217-1235] | [1217-1235] |
| 127 | AGAAUCACAGAAACACUUU | AAAGUGUUUCUGUGAUUCU | | [2123-2141] | [1832-1850] |
| 128 | AUGCUUGGCUACAGAUACU | AGUAUCUGUAGCCAAGCAU | | [2407-2425] | [2116-2134] |
| 129 | CCUGACCUCCUAUUAAAGA | UCUUUAAUAGGAGGUCAGG | | [2279-2297] | [1988-2006] |
| 130 | ACCUCCUAUUAAAGAAAAU | AUUUUCUUUAAUAGGAGGU | | [2283-2301] | [1992-2010] |
| 131 | AGGUGCUGCUCUUUGUGGU | ACCACAAAGAGCAGCACCU | | [2334-2352] | [2043-2061] |
| 132 | UGCCGUGGUCUAUAUCGAG | CUCGAUAUAGACCACGGCA | | [1079-1097] | [1079-1097] |
| 133 | CUGAGGGAAGCUCCAUAAC | GUUAUGGAGCUUCCCUCAG | | [210-228] | [210-228] |
| 134 | AGAGUAUGAGGCUCCUGCU | AGCAGGAGCCUCAUACUCU | | [1991-2009] | [1700-1718] |
| 135 | GAACACCAUGAAGGUCACA | UGUGACCUUCAUGGUGUUC | | [1562-1580] | [1367-1385] |
| 136 | UCUCGAACGGCUCAGGAUU | AAUCCUGAGCCGUUCGAGA | | [1138-1156] | [1138-1156] |
| 137 | CACAAUUCCCGGCAUUCGU | ACGAAUGCCGGGAAUUGUG | | [427-445] | [427-445] |
| 138 | AUAGUGCAUGGUCUGAUGA | UCAUCAGACCAUGCACUAU | | [2479-2497] | [2188-2206] |
| 139 | UGUGGUGGAGAAGACAGCA | UGCUGUCUUCUCCACCACA | | [1058-1076] | [1058-1076] |
| 140 | AAGGAGGGUGAAAACUUCU | AGAAGUUUUCACCCUCCUU | | [2441-2459] | [2150-2168] |
| 141 | CUCCUAUUAAAGAAAAUGA | UCAUUUUCUUUAAUAGGAG | | [2285-2303] | [1994-2012] |
| 142 | UGAGGCUCCUGCUCUGAUU | AAUCAGAGCAGGAGCCUCA | | [1997-2015] | [1706-1724] |
| 143 | AUGUGAUUUUGGCCAUUGG | CCAAUGGCCAAAAUCACAU | | [1834-1852] | [1543-1561] |
| 144 | AAUAAAAUUAUACCUAGCA | UGCUAGGUAUAAUUUUAUU | Rat, Ms | [2150-2168] | [1859-1877] |
| 145 | AGGCUAGAGGUAAAGCUGU | ACAGCUUUACCUCUAGCCU | | [2222-2240] | [1931-1949] |
| 146 | AAUCCCAGUUGGCAGUGCA | UGCACUGCCAACUGGGAUU | | [1903-1921] | [1612-1630] |
| 147 | UAGAUCACCAAGAGUAUGA | UCAUACUCUUGGUGAUCUA | | [1981-1999] | [1690-1708] |
| 148 | AUGAGUGCGGUUGCUGACA | UGUCAGCAACCGCACUCAU | | [2494-2512] | [2203-2221] |
| 149 | AAACACUGACCUUAUAUGU | ACAUAUAAGGUCAGUGUUU | | [1939-1957] | [1648-1666] |
| 150 | CUGACAUGGGUUUCUUGGU | ACCAAGAAACCCAUGUCAG | | [2507-2525] | [2216-2234] |
| 151 | CUAUUAAAGAAAAUGAGCU | AGCUCAUUUUCUUUAAUAG | | [2288-2306] | [1997-2015] |
| 152 | CUGGGAGCAAAGAUUCCCA | UGGGAAUCUUUGCUCCCAG | | [2389-2407] | [2098-2116] |
| 153 | GAUCACCAAGAGUAUGAGG | CCUCAUACUCUUGGUGAUC | | [1983-2001] | [1692-1710] |
| 154 | CCUUCUGAUCGUCUUCGAG | CUCGAAGACGAUCAGAAGG | | [1602-1620] | [1407-1425] |
| 155 | GCAGUGCUGUUGUUGUUGU | ACAACAACAACAGCACUGC | | [948-966] | [948-966] |
| 156 | ACGAGCAGUAGGAAGCAGU | ACUGCUUCCUACUGCUCGU | | [66-84] | [66-84] |
| 157 | UUAUACCUAGCAACAUAUU | AAUAUGUUGCUAGGUAUAA | | [2157-2175] | [1866-1884] |
| 158 | CUGAAGGAGGGUGAAAACU | AGUUUUCACCCUCCUUCAG | | [2438-2456] | [2147-2165] |
| 159 | CCUUUCUGGCUGAGGUUCU | AGAACCUCAGCCAGAAAGG | | [2025-2043] | [1734-1752] |
| 160 | UCACAGAAUGAAUAGAUCA | UGAUCUAUUCAUUCUGUGA | | [1969-1987] | [1678-1696] |
| 161 | UCCUAUUAAAGAAAAUGAG | CUCAUUUUCUUUAAUAGGA | | [2286-2304] | [1995-2013] |
| 162 | AGGGAAGCUCCAUAACUGC | GCAGUUAUGGAGCUUCCCU | | [213-231] | [213-231] |
| 163 | UCCGUGUGAGACUGCUAAG | CUUAGCAGUCUCACACGGA | | [1219-1237] | [1219-1237] |
| 164 | AAAAUGCUGAAGAUGUUUA | UAAACAUCUUCAGCAUUUU | Rat, Ms | [1867-1885] | [1576-1594] |
| 165 | UGAGGGAAGCUCCAUAACU | AGUUAUGGAGCUUCCCUCA | | [211-229] | [211-229] |
| 166 | GGGAAGCUCCAUAACUGCU | AGCAGUUAUGGAGCUUCCC | | [214-232] | [214-232] |
| 167 | CUUUCUGGCUGAGGUUCUG | CAGAACCUCAGCCAGAAAG | | [2026-2044] | [1735-1753] |
| 168 | UACAGAUACUGACAGCUGG | CCAGCUGUCAGUAUCUGUA | | [2416-2434] | [2125-2143] |
| 169 | CAAAGAUUCCCAUGCUUGG | CCAAGCAUGGGAAUCUUUG | | [2396-2414] | [2105-2123] |
| 170 | CCUAUUAAAGAAAAUGAGC | GCUCAUUUUCUUUAAUAGG | | [2287-2305] | [1996-2014] |
| 171 | CGUGCACUUCUGAAGGACU | AGUCCUUCAGAAGUGCACG | | [486-504] | [486-504] |
| 172 | AUAGAUCACCAAGAGUAUG | CAUACUCUUGGUGAUCUAU | | [1980-1998] | [1689-1707] |
| 173 | AAAUAAAAUUAUACCUAGC | GCUAGGUAUAAUUUUAUUU | Rat, Ms | [2149-2167] | [1858-1876] |
| 174 | UAUCUCGAACGGCUCAGGA | UCCUGAGCCGUUCGAGAUA | | [1136-1154] | [1136-1154] |
| 175 | GACCACAAUUCCCGGCAUU | AAUGCCGGGAAUUGUGGUC | | [424-442] | [424-442] |
| 176 | ACUUCAUCGCAGAUGUGGU | ACCACAUCUGCGAUGAAGU | | [1045-1063] | [1045-1063] |
| 177 | UCUUCGAGAGUUUCUGCAU | AUGCAGAAACUCUCGAAGA | | [1613-1631] | [1418-1436] |
| 178 | AGAAAAUGAGCUGCUGCCA | UGGCAGCAGCUCAUUUUCU | | [2295-2313] | [2004-2022] |
| 179 | UAAAAUUAUACCUAGCAAC | GUUGCUAGGUAUAAUUUUA | Rat | [2152-2170] | [1861-1879] |
| 180 | CCUGGUGAUGUGAUUUUGG | CCAAAAUCACAUCACCAGG | | [1827-1845] | [1536-1554] |
| 181 | GCUCCUGCUCUGAUUUCCU | AGGAAAUCAGAGCAGGAGC | | [2001-2019] | [1710-1728] |
| 182 | UAGUGCAUGGUCUGAUGAG | CUCAUCAGACCAUGCACUA | | [2480-2498] | [2189-2207] |
| 183 | UUCUGAAGGACUUCAGGUA | UACCUGAAGUCCUUCAGAA | | [493-511] | [493-511] |
| 184 | CAACGCUGAGGAUUCAGAC | GUCUGAAUCCUCAGCGUUG | | [1291-1309] | [1291-1309] |
| 185 | AUGAAUAGAUCACCAAGAG | CUCUUGGUGAUCUAUUCAU | | [1976-1994] | [1685-1703] |
| 186 | CAUCGCAACGCUGAGGAUU | AAUCCUCAGCGUUGCGAUG | | [1286-1304] | [1286-1304] |
| 187 | AACCCAAUCCCAGUUGGCA | UGCCAACUGGGAUUGGGUU | | [1898-1916] | [1607-1625] |
| 188 | CAGCUGGAAUCUCCUUUGC | GCAAAGGAGAUUCCAGCUG | Rat, Ms, GP | [1579-1597] | [1384-1402] |
| 189 | GUCACAGCUGGAAUCUCCU | AGGAGAUUCCAGCUGUGAC | GP | [1575-1593] | [1380-1398] |
| 190 | ACCUAGCAACAUAUUAUAG | CUAUAAUAUGUUGCUAGGU | | [2161-2179] | [1870-1888] |
| 191 | UAAAGAAAAUGAGCUGCUG | CAGCAGCUCAUUUUCUUUA | | [2292-2310] | [2001-2019] |
| 192 | UGCUGACAUGGGUUUCUUG | CAAGAAACCCAUGUCAGCA | | [2505-2523] | [2214-2232] |
| 193 | GUACAACUUCAUCGCAGAU | AUCUGCGAUGAAGUUGUAC | | [1040-1058] | [1040-1058] |
| 194 | AAAACUUCUGCUUGACAGU | ACUGUCAAGCAGAAGUUUU | | [2451-2469] | [2160-2178] |
| 195 | UGUGAUUUUGGCCAUUGGG | CCCAAUGGCCAAAAUCACA | | [1835-1853] | [1544-1562] |
| 196 | GUCUUCGAGAGUUUCUGCA | UGCAGAAACUCUCGAAGAC | | [1612-1630] | [1417-1435] |
| 197 | GUGCAUGGUCUGAUGAGUG | CACUCAUCAGACCAUGCAC | | [2482-2500] | [2191-2209] |
| 198 | ACUUCUGCUUGACAGUUCC | GGAACUGUCAAGCAGAAGU | | [2454-2472] | [2163-2181] |
| 199 | CUCCUUUGCCAUCCCUUCU | AGAAGGGAUGGCAAAGGAG | Rat, Ms | [1589-1607] | [1394-1412] |
| 200 | UUACCGGUGCGAGUCAAAG | CUUUGACUCGCACCGGUAA | | [172-190] | [172-190] |
| 201 | UUCUGAUCGUCUUCGAGAG | CUCUCGAAGACGAUCAGAA | | [1604-1622] | [1409-1427] |
| 202 | GAUGUGGUGGAGAAGACAG | CUGUCUUCUCCACCACAUC | Ms | [1056-1074] | [1056-1074] |
| 203 | CAACUUCAUCGCAGAUGUG | CACAUCUGCGAUGAAGUUG | | [1043-1061] | [1043-1061] |
| 204 | CACCAUGAAGGUCACAGCU | AGCUGUGACCUUCAUGGUG | | [1565-1583] | [1370-1388] |
| 205 | GAGAUACUGGAGCUGACCA | UGGUCAGCUCCAGUAUCUC | | [2259-2277] | [1968-1986] |
| 206 | AUGCUGAAGAUGUUUAUGA | UCAUAAACAUCUUCAGCAU | Rat, Ms | [1870-1888] | [1579-1597] |
| 207 | CUUGGUAAGCUCCUGAGGU | ACCUCAGGAGCUUACCAAG | | [2520-2538] | [2229-2247] |
| 208 | AGGAGGGUGAAAACUUCUG | CAGAAGUUUUCACCCUCCU | | [2442-2460] | [2151-2169] |
| 209 | UCAUUGUCACCAACGCCCA | UGGGCGUUGGUGACAAUGA | | [1177-1195] | [1177-1195] |
| 210 | GAGCAGAUGGUACAAAAUG | CAUUUUGUACCAUCUGCUC | | [1854-1872] | [1563-1581] |
| 211 | UGUGGGCAGUUAGUCAGGU | ACCUGACUAACUGCCCACA | | [2319-2337] | [2028-2046] |
| 212 | GUAAAAAAUGAGGUGGGAG | CUCCCACCUCAUUUUUUAC | | [2179-2197] | [1888-1906] |
| 213 | CGUCUUCGAGAGUUUCUGC | GCAGAAACUCUCGAAGACG | | [1611-1629] | [1416-1434] |
| 214 | AAUCACAGAAACACUUUUU | AAAAAGUGUUUCUGUGAUU | | [2125-2143] | [1834-1852] |
| 215 | AUCUUUUGUGGGCAGUUAG | CUAACUGCCCACAAAAGAU | | [2313-2331] | [2022-2040] |
| 216 | UCAGGUGCUGCUCUUUGUG | CACAAAGAGCAGCACCUGA | | [2332-2350] | [2041-2059] |
| 217 | UCCUGGGCUCUGAAGAAUC | GAUUCUUCAGAGCCCAGGA | | [2110-2128] | [1819-1837] |
| 218 | GGAAAAGAAGAAUUCCUCC | GGAGGAAUUCUUCUUUUCC | | [1637-1655] | [1442-1460] |
| 219 | AUCGCAGAUGUGGUGGAGA | UCUCCACCACAUCUGCGAU | Ms | [1050-1068] | [1050-1068] |
| 220 | UUUCUUGGUAAGCUCCUGA | UCAGGAGCUUACCAAGAAA | | [2517-2535] | [2226-2244] |
| 221 | AAUUCCUCCUCCGGAAUCA | UGAUUCCGGAGGAGGAAUU | | [1647-1665] | [1452-1470] |
| 222 | CUUCUGAAGGACUUCAGGU | ACCUGAAGUCCUUCAGAAG | | [492-510] | [492-510] |
| 223 | ACAGAUACUGACAGCUGGC | GCCAGCUGUCAGUAUCUGU | | [2417-2435] | [2126-2144] |
| 224 | GAGUCAGUACAACUUCAUC | GAUGAAGUUGUACUGACUC | | [1034-1052] | [1034-1052] |
| 225 | UCACCAAGAGUAUGAGGCU | AGCCUCAUACUCUUGGUGA | | [1985-2003] | [1694-1712] |
| 226 | CUAUAUCGAGAUCCUGGAC | GUCCAGGAUCUCGAUAUAG | | [1088-1106] | [1088-1106] |
| 227 | AUGAGGCUCCUGCUCUGAU | AUCAGAGCAGGAGCCUCAU | | [1996-2014] | [1705-1723] |
| 228 | GAUGUGAUUUUGGCCAUUG | CAAUGGCCAAAAUCACAUC | | [1833-1851] | [1542-1560] |
| 229 | CUUCGAGAGUUUCUGCAUC | GAUGCAGAAACUCUCGAAG | | [1614-1632] | [1419-1437] |
| 230 | UACAAAAUGCUGAAGAUGU | ACAUCUUCAGCAUUUUGUA | Rat | [1864-1882] | [1573-1591] |
| 231 | AGAGUUUCUGCAUCGUGGG | CCCACGAUGCAGAAACUCU | | [1619-1637] | [1424-1442] |
| 232 | CAGAUGGUACAAAAUGCUG | CAGCAUUUUGUACCAUCUG | | [1857-1875] | [1566-1584] |
| 233 | UUAUAGUAAAAAAUGAGGU | ACCUCAUUUUUUACUAUAA | | [2174-2192] | [1883-1901] |
| 234 | AGGUCACAGCUGGAAUCUC | GAGAUUCCAGCUGUGACCU | GP | [1573-1591] | [1378-1396] |
| 235 | GCUGACAUGGGUUUCUUGG | CCAAGAAACCCAUGUCAGC | | [2506-2524] | [2215-2233] |
| 236 | AUGGUCUGAUGAGUGCGGU | ACCGCACUCAUCAGACCAU | | [2486-2504] | [2195-2213] |
| 237 | CUGGUGAUGUGAUUUUGGC | GCCAAAAUCACAUCACCAG | | [1828-1846] | [1537-1555] |
| 238 | GCUUGACAGUUCCACAUCC | GGAUGUGGAACUGUCAAGC | | [2460-2478] | [2169-2187] |
| 239 | GGUCUAUAUCGAGAUCCUG | CAGGAUCUCGAUAUAGACC | | [1085-1103] | [1085-1103] |
| 240 | GUACAAAAUGCUGAAGAUG | CAUCUUCAGCAUUUUGUAC | | [1863-1881] | [1572-1590] |
| 241 | GAAUCUCCUUUGCCAUCCC | GGGAUGGCAAAGGAGAUUC | Rat, Ms, GP | [1585-1603] | [1390-1408] |
| 242 | GUCUAUAUCGAGAUCCUGG | CCAGGAUCUCGAUAUAGAC | | [1086-1104] | [1086-1104] |
| 243 | AAAAUGAGGUGGGAGGGCU | AGCCCUCCCACCUCAUUUU | | [2183-2201] | [1892-1910] |
| 244 | GUGCUGUUGUUGUUGUGGG | CCCACAACAACAACAGCAC | | [951-969] | [951-969] |
| 245 | UCAGUACAACUUCAUCGCA | UGCGAUGAAGUUGUACUGA | | [1037-1055] | [1037-1055] |
| 246 | AAAAUGAGCUGCUGCCAUC | GAUGGCAGCAGCUCAUUUU | | [2297-2315] | [2006-2024] |
| 247 | AAGGCUAGAGGUAAAGCUG | CAGCUUUACCUCUAGCCUU | | [2221-2239] | [1930-1948] |
| 248 | AUCUCGAACGGCUCAGGAU | AUCCUGAGCCGUUCGAGAU | | [1137-1155] | [1137-1155] |
| 249 | AGCUGCUGCCAUCUUUUGU | ACAAAAGAUGGCAGCAGCU | | [2303-2321] | [2012-2030] |
| 250 | AGAUACUGACAGCUGGCCU | AGGCCAGCUGUCAGUAUCU | | [2419-2437] | [2128-2146] |
| 251 | GGUUGCUGACAUGGGUUUC | GAAACCCAUGUCAGCAACC | | [2502-2520] | [2211-2229] |
| 252 | UAUUAAAGAAAAUGAGCUG | CAGCUCAUUUUCUUUAAUA | | [2289-2307] | [1998-2016] |
| 253 | UUGCCUUUCUGGCUGAGGU | ACCUCAGCCAGAAAGGCAA | | [2022-2040] | [1731-1749] |
| 254 | CUGACGUCAGGAACUUCUG | CAGAAGUUCCUGACGUCAG | | [717-735] | [717-735] |
| 255 | GGUUUCUUGGUAAGCUCCU | AGGAGCUUACCAAGAAACC | | [2515-2533] | [2224-2242] |
| 256 | GUUAGUCAGGUGCUGCUCU | AGAGCAGCACCUGACUAAC | | [2327-2345] | [2036-2054] |
| 257 | UGGUGAUGUGAUUUUGGCC | GGCCAAAAUCACAUCACCA | | [1829-1847] | [1538-1556] |
| 258 | ACAGUUCCACAUCCAUAGU | ACUAUGGAUGUGGAACUGU | | [2465-2483] | [2174-2192] |
| 259 | UCUGAUGAGUGCGGUUGCU | AGCAACCGCACUCAUCAGA | | [2490-2508] | [2199-2217] |
| 260 | GAGCUGCUGCCAUCUUUUG | CAAAAGAUGGCAGCAGCUC | | [2302-2320] | [2011-2029] |
| 261 | GAUUCCCAUGCUUGGCUAC | GUAGCCAAGCAUGGGAAUC | | [2400-2418] | [2109-2127] |
| 262 | UUAGUCAGGUGCUGCUCUU | AAGAGCAGCACCUGACUAA | | [2328-2346] | [2037-2055] |
| 263 | GCUCCAUAACUGCUGCUUC | GAAGCAGCAGUUAUGGAGC | | [219-237] | [219-237] |
| 264 | UAUGAGGCUCCUGCUCUGA | UCAGAGCAGGAGCCUCAUA | | [1995-2013] | [1704-1722] |
| 265 | GAAGCUCCAUAACUGCUGC | GCAGCAGUUAUGGAGCUUC | | [216-234] | [216-234] |
| 266 | GAUACUGGAGCUGACCAUC | GAUGGUCAGCUCCAGUAUC | | [2261-2279] | [1970-1988] |
| 267 | AUGAAGCUGUUCGAACCCA | UGGGUUCGAACAGCUUCAU | Ms | [1885-1903] | [1594-1612] |
| 268 | GUUUCUUGGUAAGCUCCUG | CAGGAGCUUACCAAGAAAC | | [2516-2534] | [2225-2243] |
| 269 | UACUGGAGCUGACCAUCCU | AGGAUGGUCAGCUCCAGUA | | [2263-2281] | [1972-1990] |
| 270 | AGCUGUUCGAACCCAAUCC | GGAUUGGGUUCGAACAGCU | | [1889-1907] | [1598-1616] |
| 271 | AAAGGCUAGAGGUAAAGCU | AGCUUUACCUCUAGCCUUU | | [2220-2238] | [1929-1947] |
| 272 | AAAUGAGCUGCUGCCAUCU | AGAUGGCAGCAGCUCAUUU | | [2298-2316] | [2007-2025] |
| 273 | UAUAGUAAAAAAUGAGGUG | CACCUCAUUUUUUACUAUA | | [2175-2193] | [1884-1902] |
| 274 | CUGGGCUCUGAAGAAUCAC | GUGAUUCUUCAGAGCCCAG | | [2112-2130] | [1821-1839] |
| 275 | AAGCUGUUCGAACCCAAUC | GAUUGGGUUCGAACAGCUU | Ms | [1888-1906] | [1597-1615] |
| 276 | GUUGCUGACAUGGGUUUCU | AGAAACCCAUGUCAGCAAC | | [2503-2521] | [2212-2230] |
| 277 | UUAUGAAGCUGUUCGAACC | GGUUCGAACAGCUUCAUAA | Ms | [1883-1901] | [1592-1610] |
| 278 | ACACCAUGAAGGUCACAGC | GCUGUGACCUUCAUGGUGU | | [1564-1582] | [1369-1387] |
| 279 | UCCUGACCUCCUAUUAAAG | CUUUAAUAGGAGGUCAGGA | | [2278-2296] | [1987-2005] |
| 280 | ACAAUUCCCGGCAUUCGUG | CACGAAUGCCGGGAAUUGU | | [428-446] | [428-446] |
| 281 | CUCAGAGAACUCUGGAACC | GGUUCCAGAGUUCUCUGAG | | [887-905] | [887-905] |
| 282 | UUAAAGAAAAUGAGCUGCU | AGCAGCUCAUUUUCUUUAA | | [2291-2309] | [2000-2018] |
| 283 | UCUCCUUUGCCAUCCCUUC | GAAGGGAUGGCAAAGGAGA | Rat, Ms | [1588-1606] | [1393-1411] |
| 284 | UAAAAAAUGAGGUGGGAGG | CCUCCCACCUCAUUUUUUA | | [2180-2198] | [1889-1907] |
| 285 | AGCAGUAGGAAGCAGUCAC | GUGACUGCUUCCUACUGCU | | [69-87] | [69-87] |
| 286 | UUUCUGGCUGAGGUUCUGA | UCAGAACCUCAGCCAGAAA | | [2027-2045] | [1736-1754] |
| 287 | CCAUAACUGCUGCUUCAGG | CCUGAAGCAGCAGUUAUGG | | [222-240] | [222-240] |
| 288 | GGGUUAAAUGAACCAGUGG | CCACUGGUUCAUUUAACCC | | [2061-2079] | [1770-1788] |
| 289 | UCUGAUUUCCUCCUUGCCU | AGGCAAGGAGGAAAUCAGA | | [2009-2027] | [1718-1736] |
| 290 | UUCGAACCCAAUCCCAGUU | AACUGGGAUUGGGUUCGAA | | [1894-1912] | [1603-1621] |
| 291 | GAUCGUCUUCGAGAGUUUC | GAAACUCUCGAAGACGAUC | | [1608-1626] | [1413-1431] |
| 292 | ACUUCUGAAGGACUUCAGG | CCUGAAGUCCUUCAGAAGU | | [491-509] | [491-509] |
| 293 | AACACUGACCUUAUAUGUG | CACAUAUAAGGUCAGUGUU | | [1940-1958] | [1649-1667] |
| 294 | AUGGGUUUCUUGGUAAGCU | AGCUUACCAAGAAACCCAU | | [2512-2530] | [2221-2239] |
| 295 | AUCUCCUUUGCCAUCCCUU | AAGGGAUGGCAAAGGAGAU | Rat, Ms | [1587-1605] | [1392-1410] |
| 296 | GAUGUUUAUGAAGCUGUUC | GAACAGCUUCAUAAACAUC | Ms | [1878-1896] | [1587-1605] |
| 297 | UCCAUAGUGCAUGGUCUGA | UCAGACCAUGCACUAUGGA | | [2476-2494] | [2185-2203] |
| 298 | UCGUCUUCGAGAGUUUCUG | CAGAAACUCUCGAAGACGA | | [1610-1628] | [1415-1433] |
| 299 | AGAAGAAUUCCUCCUCCGG | CCGGAGGAGGAAUUCUUCU | | [1642-1660] | [1447-1465] |
| 300 | AGCAGAUGGUACAAAAUGC | GCAUUUUGUACCAUCUGCU | | [1855-1873] | [1564-1582] |
| 301 | CUACAGAUACUGACAGCUG | CAGCUGUCAGUAUCUGUAG | | [2415-2433] | [2124-2142] |
| 302 | AAUCUCCUUUGCCAUCCCU | AGGGAUGGCAAAGGAGAUU | Rat, Ms | [1586-1604] | [1391-1409] |
| 303 | AUUAUAGUAAAAAAUGAGG | CCUCAUUUUUUACUAUAAU | | [2173-2191] | [1882-1900] |
| 304 | AAGAUGUUUAUGAAGCUGU | ACAGCUUCAUAAACAUCUU | Ms | [1876-1894] | [1585-1603] |
| 305 | UAGUAAAAAAUGAGGUGGG | CCCACCUCAUUUUUUACUA | | [2177-2195] | [1886-1904] |
| 306 | UUCUGGCUGAGGUUCUGAG | CUCAGAACCUCAGCCAGAA | | [2028-2046] | [1737-1755] |
| 307 | AAACUUCUGCUUGACAGUU | AACUGUCAAGCAGAAGUUU | | [2452-2470] | [2161-2179] |
| 308 | UUGUGGGCAGUUAGUCAGG | CCUGACUAACUGCCCACAA | | [2318-2336] | [2027-2045] |
| 309 | UUUGUGGGCAGUUAGUCAG | CUGACUAACUGCCCACAAA | | [2317-2335] | [2026-2044] |
| 310 | AAUGCUGAAGAUGUUUAUG | CAUAAACAUCUUCAGCAUU | Rat, Ms | [1869-1887] | [1578-1596] |
| 311 | UGCUGACGUCAGGAACUUC | GAAGUUCCUGACGUCAGCA | | [715-733] | [715-733] |
| 312 | GGUGAUUGGAGUGAACACC | GGUGUUCACUCCAAUCACC | Rat, Ms | [1550-1568] | [1355-1373] |
| 313 | AUUUCCUCCUUGCCUUUCU | AGAAAGGCAAGGAGGAAAU | | [2013-2031] | [1722-1740] |
| 314 | CUUUUGUGGGCAGUUAGUC | GACUAACUGCCCACAAAAG | | [2315-2333] | [2024-2042] |
| 315 | CUCUGAUUUCCUCCUUGCC | GGCAAGGAGGAAAUCAGAG | | [2008-2026] | [1717-1735] |
| 316 | CUUCAUCGCAGAUGUGGUG | CACCACAUCUGCGAUGAAG | | [1046-1064] | [1046-1064] |
| 317 | CAUGGGUUUCUUGGUAAGC | GCUUACCAAGAAACCCAUG | | [2511-2529] | [2220-2238] |
| 318 | AAGGUCACAGCUGGAAUCU | AGAUUCCAGCUGUGACCUU | GP | [1572-1590] | [1377-1395] |
| 319 | AUGUGGUGGAGAAGACAGC | GCUGUCUUCUCCACCACAU | Ms | [1057-1075] | [1057-1075] |
| 320 | AACGCUGAGGAUUCAGACU | AGUCUGAAUCCUCAGCGUU | | [1292-1310] | [1292-1310] |
| 321 | AUCCAUAGUGCAUGGUCUG | CAGACCAUGCACUAUGGAU | | [2475-2493] | [2184-2202] |
| 322 | UCUUGGUAAGCUCCUGAGG | CCUCAGGAGCUUACCAAGA | | [2519-2537] | [2228-2246] |
| 323 | AUUCCCAUGCUUGGCUACA | UGUAGCCAAGCAUGGGAAU | | [2401-2419] | [2110-2128] |
| 324 | GAUGGGCUCAUUGUCACCA | UGGUGACAAUGAGCCCAUC | | [1170-1188] | [1170-1188] |
| 325 | CUGAAGAUGUUUAUGAAGC | GCUUCAUAAACAUCUUCAG | Ms | [1873-1891] | [1582-1600] |
| 326 | UUCCCAUGCUUGGCUACAG | CUGUAGCCAAGCAUGGGAA | | [2402-2420] | [2111-2129] |
| 327 | UCUGAUCGUCUUCGAGAGU | ACUCUCGAAGACGAUCAGA | | [1605-1623] | [1410-1428] |
| 328 | AAUGAGCUGCUGCCAUCUU | AAGAUGGCAGCAGCUCAUU | | [2299-2317] | [2008-2026] |
| 329 | AUAACUGCUGCUUCAGGAG | CUCCUGAAGCAGCAGUUAU | | [224-242] | [224-242] |
| 330 | CUAGAGGUAAAGCUGUAUC | GAUACAGCUUUACCUCUAG | | [2225-2243] | [1934-1952] |
| 331 | AAAAAAUGAGGUGGGAGGG | CCCUCCCACCUCAUUUUUU | | [2181-2199] | [1890-1908] |
| 332 | AGUUCCACAUCCAUAGUGC | GCACUAUGGAUGUGGAACU | | [2467-2485] | [2176-2194] |
| 333 | AGAACUCUGGAACCCGUUC | GAACGGGUUCCAGAGUUCU | | [892-910] | [892-910] |
| 334 | UGCUUGGCUACAGAUACUG | CAGUAUCUGUAGCCAAGCA | | [2408-2426] | [2117-2135] |
| 335 | AGUAUGAGGCUCCUGCUCU | AGAGCAGGAGCCUCAUACU | | [1993-2011] | [1702-1720] |
| 336 | CAAGAGUAUGAGGCUCCUG | CAGGAGCCUCAUACUCUUG | | [1989-2007] | [1698-1716] |
| 337 | AAGAAGAAUUCCUCCUCCG | CGGAGGAGGAAUUCUUCUU | | [1641-1659] | [1446-1464] |
| 338 | AAAGAAGAAUUCCUCCUCC | GGAGGAGGAAUUCUUCUUU | | [1640-1658] | [1445-1463] |
| 339 | GGUUAAAUGAACCAGUGGG | CCCACUGGUUCAUUUAACC | | [2062-2080] | [1771-1789] |
| 340 | AGUCAGGUGCUGCUCUUUG | CAAAGAGCAGCACCUGACU | | [2330-2348] | [2039-2057] |
| 341 | UUUCCUCCUUGCCUUUCUG | CAGAAAGGCAAGGAGGAAA | | [2014-2032] | [1723-1741] |
| 342 | UAGUCAGGUGCUGCUCUUU | AAAGAGCAGCACCUGACUA | | [2329-2347] | [2038-2056] |
| 343 | UGCAUGGUCUGAUGAGUGC | GCACUCAUCAGACCAUGCA | | [2483-2501] | [2192-2210] |
| 344 | ACAGAGGGUUAAAUGAACC | GGUUCAUUUAACCCUCUGU | | [2056-2074] | [1765-1783] |
| 345 | CUGCUCUUUGUGGUGUGGU | ACCACACCACAAAGAGCAG | | [2339-2357] | [2048-2066] |
| 346 | CACCAAGAGUAUGAGGCUC | GAGCCUCAUACUCUUGGUG | | [1986-2004] | [1695-1713] |
| 347 | GGGUUUCUUGGUAAGCUCC | GGAGCUUACCAAGAAACCC | | [2514-2532] | [2223-2241] |
| 348 | GACGUCAGGAACUUCUGAC | GUCAGAAGUUCCUGACGUC | | [719-737] | [719-737] |
| 349 | CUGCUGCCAUCUUUUGUGG | CCACAAAAGAUGGCAGCAG | | [2305-2323] | [2014-2032] |
| 350 | AACUUCUGCUUGACAGUUC | GAACUGUCAAGCAGAAGUU | | [2453-2471] | [2162-2180] |
| 351 | UUCUUGGUAAGCUCCUGAG | CUCAGGAGCUUACCAAGAA | | [2518-2536] | [2227-2245] |
| 352 | ACAUCCAUAGUGCAUGGUC | GACCAUGCACUAUGGAUGU | | [2473-2491] | [2182-2200] |
| 353 | UUCCACAUCCAUAGUGCAU | AUGCACUAUGGAUGUGGAA | | [2469-2487] | [2178-2196] |
| 354 | UCCACAUCCAUAGUGCAUG | CAUGCACUAUGGAUGUGGA | | [2470-2488] | [2179-2197] |
| 355 | UUUAUGAAGCUGUUCGAAC | GUUCGAACAGCUUCAUAAA | Ms | [1882-1900] | [1591-1609] |
| 356 | AGUGCUGUUGUUGUUGUGG | CCACAACAACAACAGCACU | | [950-968] | [950-968] |
| 357 | ACAGAAUGAAUAGAUCACC | GGUGAUCUAUUCAUUCUGU | | [1971-1989] | [1680-1698] |
| 358 | AUACUGACAGCUGGCCUCU | AGAGGCCAGCUGUCAGUAU | | [2421-2439] | [2130-2148] |
| 359 | UAGAGGUAAAGCUGUAUCC | GGAUACAGCUUUACCUCUA | | [2226-2244] | [1935-1953] |
| 360 | UCGAACCCAAUCCCAGUUG | CAACUGGGAUUGGGUUCGA | | [1895-1913] | [1604-1622] |
| 361 | CUGAAGGACUUCAGGUACC | GGUACCUGAAGUCCUUCAG | | [495-513] | [495-513] |
| 362 | AAGAGUAUGAGGCUCCUGC | GCAGGAGCCUCAUACUCUU | | [1990-2008] | [1699-1717] |
| 363 | ACGUCAGGAACUUCUGACC | GGUCAGAAGUUCCUGACGU | | [720-738] | [720-738] |
| 364 | UAAGCUCCUGAGGUAAUGG | CCAUUACCUCAGGAGCUUA | Rat | [2525-2543] | [2234-2252] |
| 365 | UUGUCACCAACGCCCAUGU | ACAUGGGCGUUGGUGACAA | | [1180-1198] | [1180-1198] |
| 366 | UCUGAAGGACUUCAGGUAC | GUACCUGAAGUCCUUCAGA | | [494-512] | [494-512] |
| 367 | AUCGCAACGCUGAGGAUUC | GAAUCCUCAGCGUUGCGAU | | [1287-1305] | [1287-1305] |
| 368 | UGAAGGACUUCAGGUACCG | CGGUACCUGAAGUCCUUCA | | [496-514] | [496-514] |
| 369 | AAAAGAAGAAUUCCUCCUC | GAGGAGGAAUUCUUCUUUU | | [1639-1657] | [1444-1462] |
| 370 | UUCCUCCUCCGGAAUCAGU | ACUGAUUCCGGAGGAGGAA | | [1649-1667] | [1454-1472] |
| 371 | AAGAUUCCCAUGCUUGGCU | AGCCAAGCAUGGGAAUCUU | | [2398-2416] | [2107-2125] |
| 372 | AUCCCUUCUGAUCGUCUUC | GAAGACGAUCAGAAGGGAU | | [1599-1617] | [1404-1422] |
| 373 | GAAUUCCUCCUCCGGAAUC | GAUUCCGGAGGAGGAAUUC | | [1646-1664] | [1451-1469] |
| 374 | AAAGAAAAUGAGCUGCUGC | GCAGCAGCUCAUUUUCUUU | | [2293-2311] | [2002-2020] |
| 375 | AGCUGGAAUCUCCUUUGCC | GGCAAAGGAGAUUCCAGCU | Rat, Ms, GP | [1580-1598] | [1385-1403] |
| 376 | AAGAAAAUGAGCUGCUGCC | GGCAGCAGCUCAUUUUCUU | | [2294-2312] | [2003-2021] |
| 377 | AUUCCUCCUCCGGAAUCAG | CUGAUUCCGGAGGAGGAAU | | [1648-1666] | [1453-1471] |
| 378 | UGGAAUCUCCUUUGCCAUC | GAUGGCAAAGGAGAUUCCA | Rat, Ms, GP | [1583-1601] | [1388-1406] |
| 379 | UUUGGAAACUCUGGAGGUC | GACCUCCAGAGUUUCCAAA | GP, Chn | [1509-1527] | [1314-1332] |
| 380 | GUCAGUACAACUUCAUCGC | GCGAUGAAGUUGUACUGAC | | [1036-1054] | [1036-1054] |
| 381 | UGCUUGACAGUUCCACAUC | GAUGUGGAACUGUCAAGCA | | [2459-2477] | [2168-2186] |
| 382 | ACCACAAUUCCCGGCAUUC | GAAUGCCGGGAAUUGUGGU | | [425-443] | [425-443] |
| 383 | AACACCAUGAAGGUCACAG | CUGUGACCUUCAUGGUGUU | | [1563-1581] | [1368-1386] |
| 384 | ACCUGCCGUGGUCUAUAUC | GAUAUAGACCACGGCAGGU | | [1076-1094] | [1076-1094] |
| 385 | UUCUGCUUGACAGUUCCAC | GUGGAACUGUCAAGCAGAA | | [2456-2474] | [2165-2183] |
| 386 | AAAAAUGAGGUGGGAGGGC | GCCCUCCCACCUCAUUUUU | | [2182-2200] | [1891-1909] |
| 387 | AUAUCGAGAUCCUGGACCG | CGGUCCAGGAUCUCGAUAU | Chn | [1090-1108] | [1090-1108] |
| 388 | AUGUUUAUGAAGCUGUUCG | CGAACAGCUUCAUAAACAU | Ms | [1879-1897] | [1588-1606] |
| 389 | AAGCUCCUGAGGUAAUGGC | GCCAUUACCUCAGGAGCUU | Rat | [2526-2544] | [2235-2253] |
| 390 | AAGCUCCAUAACUGCUGCU | AGCAGCAGUUAUGGAGCUU | | [217-235] | [217-235] |
| 391 | AGUCAGUACAACUUCAUCG | CGAUGAAGUUGUACUGACU | | [1035-1053] | [1035-1053] |
| 392 | UAUGAAGCUGUUCGAACCC | GGGUUCGAACAGCUUCAUA | Ms | [1884-1902] | [1593-1611] |
| 393 | ACCAUGAAGGUCACAGCUG | CAGCUGUGACCUUCAUGGU | | [1566-1584] | [1371-1389] |
| 394 | AGUGAACACCAUGAAGGUC | GACCUUCAUGGUGUUCACU | | [1559-1577] | [1364-1382] |
| 395 | UCCAUAACUGCUGCUUCAG | CUGAAGCAGCAGUUAUGGA | | [221-239] | [221-239] |
| 396 | UUCGAGAGUUUCUGCAUCG | CGAUGCAGAAACUCUCGAA | | [1615-1633] | [1420-1438] |
| 397 | AGAGGUAAAGCUGUAUCCC | GGGAUACAGCUUUACCUCU | | [2227-2245] | [1936-1954] |
| 398 | AUGGUACAAAAUGCUGAAG | CUUCAGCAUUUUGUACCAU | | [1860-1878] | [1569-1587] |
| 399 | AGUUAGUCAGGUGCUGCUC | GAGCAGCACCUGACUAACU | | [2326-2344] | [2035-2053] |
| 400 | UCCUGCUCUGAUUUCCUCC | GGAGGAAAUCAGAGCAGGA | | [2003-2021] | [1712-1730] |
| 401 | UGCACUUCUGAAGGACUUC | GAAGUCCUUCAGAAGUGCA | | [488-506] | [488-506] |
| 402 | AUUAAAGAAAAUGAGCUGC | GCAGCUCAUUUUCUUUAAU | | [2290-2308] | [1999-2017] |
| 403 | AUAGUAAAAAAUGAGGUGG | CCACCUCAUUUUUUACUAU | | [2176-2194] | [1885-1903] |
| 404 | GAUUUCCUCCUUGCCUUUC | GAAAGGCAAGGAGGAAAUC | | [2012-2030] | [1721-1739] |
| 405 | AUCGUCUUCGAGAGUUUCU | AGAAACUCUCGAAGACGAU | | [1609-1627] | [1414-1432] |
| 406 | AAAAGGCUAGAGGUAAAGC | GCUUUACCUCUAGCCUUUU | | [2219-2237] | [1928-1946] |
| 407 | UGCUGCCAUCUUUUGUGGG | CCCACAAAAGAUGGCAGCA | | [2306-2324] | [2015-2033] |
| 408 | UUGCCAUCCCUUCUGAUCG | CGAUCAGAAGGGAUGGCAA | Rat, Ms | [1594-1612] | [1399-1417] |
| 409 | AACUUCAUCGCAGAUGUGG | CCACAUCUGCGAUGAAGUU | | [1044-1062] | [1044-1062] |
| 410 | UCCCUUCUGAUCGUCUUCG | CGAAGACGAUCAGAAGGGA | | [1600-1618] | [1405-1423] |
| 411 | UCAGAGAACUCUGGAACCC | GGGUUCCAGAGUUCUCUGA | | [888-906] | [888-906] |
| 412 | AAAGAUUCCCAUGCUUGGC | GCCAAGCAUGGGAAUCUUU | | [2397-2415] | [2106-2124] |
| 413 | ACCAAGAGUAUGAGGCUCC | GGAGCCUCAUACUCUUGGU | | [1987-2005] | [1696-1714] |
| 414 | UUUGCCAUCCCUUCUGAUC | GAUCAGAAGGGAUGGCAAA | Rat, Ms | [1593-1611] | [1398-1416] |
| 415 | AGGCUCCUGCUCUGAUUUC | GAAAUCAGAGCAGGAGCCU | | [1999-2017] | [1708-1726] |
| 416 | CUGACCUUAUAUGUGACCC | GGGUCACAUAUAAGGUCAG | | [1944-1962] | [1653-1671] |
| 417 | UUCCUCCUUGCCUUUCUGG | CCAGAAAGGCAAGGAGGAA | | [2015-2033] | [1724-1742] |
| 418 | AUACUGGAGCUGACCAUCC | GGAUGGUCAGCUCCAGUAU | | [2262-2280] | [1971-1989] |
| 419 | AUCACCAAGAGUAUGAGGC | GCCUCAUACUCUUGGUGAU | | [1984-2002] | [1693-1711] |
| 420 | AUGAUGCUGACCCUGAGUC | GACUCAGGGUCAGCAUCAU | | [1695-1713] | [1500-1518] |
| 421 | ACUGACCUUAUAUGUGACC | GGUCACAUAUAAGGUCAGU | | [1943-1961] | [1652-1670] |
| 422 | UUGGAAACUCUGGAGGUCC | GGACCUCCAGAGUUUCCAA | GP, Chn | [1510-1528] | [1315-1333] |
| 423 | UAACUGCUGCUUCAGGAGC | GCUCCUGAAGCAGCAGUUA | | [225-243] | [225-243] |
| 424 | GUGGUCUAUAUCGAGAUCC | GGAUCUCGAUAUAGACCAC | Rat, Ms | [1083-1101] | [1083-1101] |
| 425 | UACAACUUCAUCGCAGAUG | CAUCUGCGAUGAAGUUGUA | | [1041-1059] | [1041-1059] |
| 426 | AUUGUCACCAACGCCCAUG | CAUGGGCGUUGGUGACAAU | | [1179-1197] | [1179-1197] |
| 427 | UAUAUCGAGAUCCUGGACC | GGUCCAGGAUCUCGAUAUA | Chn | [1089-1107] | [1089-1107] |
| 428 | UUCAUCGCAGAUGUGGUGG | CCACCACAUCUGCGAUGAA | Ms | [1047-1065] | [1047-1065] |

TABLE B5
KEAP1 19-mer oligomer pairs kelch-like ECH-associated protein 1

| | | | | Human | Human |
|---|---|---|---|---|---|
| | | | | 45269144: | 45269143: |
| No. | Sense | AntiSense | Other Sp | 186-2060 | 157-2031 |

| | | | | | |
|---|---|---|---|---|---|
| 1 | GGAACGAGUGGCGAAUGAU | AUCAUUCGCCACUCGUUCC | CHMP | [1666-1684] | [1637-1655] |
| 2 | GGGCAAAAAUACAGUCCAA | UUGGACUGUAUUUUUGCCC | | [2077-2095] | [2048-2066] |
| 3 | AGAGGAACGAGUGGCGAAU | AUUCGCCACUCGUUCCUCU | CHMP | [1663-1681] | [1634-1652] |
| 4 | GGAGUAUCAUUGUUUUUGU | ACAAAAACAAUGAUACUCC | CHMP | [2099-2117] | [2070-2088] |
| 5 | GCCUCAUUGAAUUCGCCUA | UAGGCGAAUUCAAUGAGGC | | [589-607] | [560-578] |
| 6 | GGCAAAAAUACAGUCCAAU | AUUGGACUGUAUUUUUGCC | | [2078-2096] | [2049-2067] |
| 7 | GGAUGCCUCAGUGUUAAAA | UUUUAACACUGAGGCAUCC | CHMP | [2183-2201] | [2154-2172] |
| 8 | CGGGACUAAAAGAAAAGAC | GUCUUUUCUUUUAGUCCCG | CHMP | [2126-2144] | [2097-2115] |
| 9 | UCAUUGAAUUCGCCUACAC | GUGUAGGCGAAUUCAAUGA | | [592-610] | [563-581] |
| 10 | GCACUGCAAAUAACCCAUC | GAUGGGUUAUUUGCAGUGC | CHMP | [2146-2164] | [2117-2135] |
| 11 | GGGACUAAAAGAAAAGACA | UGUCUUUUCUUUUAGUCCC | CHMP | [2127-2145] | [2098-2116] |
| 12 | CCGGGACUAAAAGAAAAGA | UCUUUUCUUUUAGUCCCGG | CHMP | [2125-2143] | [2096-2114] |
| 13 | AGAAAAGACAGCACUGCAA | UUGCAGUGCUGUCUUUUCU | CHMP | [2136-2154] | [2107-2125] |
| 14 | CUCAUUGAAUUCGCCUACA | UGUAGGCGAAUUCAAUGAG | | [591-609] | [562-580] |
| 15 | UGAUAAGUAACCCUGUAAU | AUUACAGGGUUACUUAUCA | CHMP | [2528-2546] | [2499-2517] |
| 16 | UGGUGGUGUUGCUUAUCUU | AAGAUAAGCAACACCACCA | | [157-175] | [128-146] |
| 17 | GGGAGUAUCAUUGUUUUUG | CAAAAACAAUGAUACUCCC | CHMP | [2098-2116] | [2069-2087] |
| 18 | UGUUGCUUAUCUUCUGGAA | UUCCAGAAGAUAAGCAACA | CHMP | [163-181] | [134-152] |
| 19 | CCUCAUUGAAUUCGCCUAC | GUAGGCGAAUUCAAUGAGG | | [590-608] | [561-579] |
| 20 | CACCAUGUGAUUUAUUCUU | AAGAAUAAAUCACAUGGUG | | [2381-2399] | [2352-2370] |
| 21 | ACUGCAAAUAACCCAUCUU | AAGAUGGGUUAUUUGCAGU | CHMP | [2148-2166] | [2119-2137] |
| 22 | CUAAAAGAAAAGACAGCAC | GUGCUGUCUUUUCUUUUAG | CHMP | [2131-2149] | [2102-2120] |
| 23 | GCAAAAAUACAGUCCAAUG | CAUUGGACUGUAUUUUUGC | | [2079-2097] | [2050-2068] |
| 24 | UCCUGCACAACUGUAUCUA | UAGAUACAGUUGUGCAGGA | CHMP | [1726-1744] | [1697-1715] |
| 25 | CCCGGGAGUACAUCUACAU | AUGUAGAUGUACUCCCGGG | CHMP | [793-811] | [764-782] |
| 26 | CUGUCUUCAAGGCCAUGUU | AACAUGGCCUUGAAGACAG | CHMP | [499-517] | [470-488] |
| 27 | AGAACAGACUAACUAGUGU | ACACUAGUUAGUCUGUUCU | CHMP | [2563-2581] | [2534-2552] |
| 28 | CACUGCAAAUAACCCAUCU | AGAUGGGUUAUUUGCAGUG | CHMP | [2147-2165] | [2118-2136] |
| 29 | GGACUAAAAGAAAAGACAG | CUGUCUUUUCUUUUAGUCC | CHMP | [2128-2146] | [2099-2117] |
| 30 | ACCGGGACUAAAAGAAAAG | CUUUUCUUUUAGUCCCGGU | CHMP | [2124-2142] | [2095-2113] |
| 31 | CCUGCACAACUGUAUCUAU | AUAGAUACAGUUGUGCAGG | CHMP | [1727-1745] | [1698-1716] |
| 32 | CCUCAAUCGUCUCCUUUAU | AUAAAGGAGACGAUUGAGG | | [1586-1604] | [1557-1575] |
| 33 | UCAUGUACCAGAUCGACAG | CUGUCGAUCUGGUACAUGA | CHMP | [664-682] | [635-653] |
| 34 | GUGUUGCUUAUCUUCUGGA | UCCAGAAGAUAAGCAACAC | CHMP | [162-180] | [133-151] |
| 35 | UGGUGUUGCUUAUCUUCUG | CAGAAGAUAAGCAACACCA | CHMP | [160-178] | [131-149] |
| 36 | CCAUGUGAUUUAUUCUUGG | CCAAGAAUAAAUCACAUGG | | [2383-2401] | [2354-2372] |
| 37 | GCAGCUGUCACCAUGUGAU | AUCACAUGGUGACAGCUGC | | [2373-2391] | [2344-2362] |
| 38 | CAUCUCAAAAGAAGUCCAA | UUGGACUUCUUUUGAGAUG | CHMP | [2205-2223] | [2176-2194] |
| 39 | AGCACUGCAAAUAACCCAU | AUGGGUUAUUUGCAGUGCU | CHMP | [2145-2163] | [2116-2134] |
| 40 | AACCGGGACUAAAAGAAAA | UUUUCUUUUAGUCCCGGUU | CHMP | [2123-2141] | [2094-2112] |
| 41 | ACAAAAACCGGGACUAAAA | UUUUAGUCCCGGUUUUUGU | CHMP | [2118-2136] | [2089-2107] |
| 42 | GUACAAAAACCGGGACUAA | UUAGUCCCGGUUUUUGUAC | CHMP | [2116-2134] | [2087-2105] |
| 43 | UGAGGCACUUUUGUUUCUU | AAGAAACAAAAGUGCCUCA | CHMP | [2058-2076] | [2029-2047] |
| 44 | CCUUAAUUCAGCUGAGUGU | ACACUCAGCUGAAUUAAGG | CHMP | [1634-1652] | [1605-1623] |
| 45 | ACAGUGUGGAGAGGUAUGA | UCAUACCUCUCCACACUGU | CHMP | [1498-1516] | [1469-1487] |
| 46 | AGGUGGUGGUGUUGCUUAU | AUAAGCAACACCACCACCU | | [154-172] | [125-143] |
| 47 | CGCCUCAUUGAAUUCGCCU | AGGCGAAUUCAAUGAGGCG | | [588-606] | [559-577] |
| 48 | GUGUUAAAAUGACAUCUCA | UGAGAUGUCAUUUUAACAC | CHMP | [2193-2211] | [2164-2182] |
| 49 | AGGAUGCCUCAGUGUUAAA | UUUAACACUGAGGCAUCCU | CHMP | [2182-2200] | [2153-2171] |
| 50 | ACGUCACACUGCAGGUCAA | UUGACCUGCAGUGUGACGU | CHMP | [418-436] | [389-407] |
| 51 | CGAUGUGGAAACAGAGACG | CGUCUCUGUUUCCACAUCG | CHMP | [1796-1814] | [1767-1785] |
| 52 | CGCUACGAUGUGGAAACAG | CUGUUUCCACAUCGUAGCG | CHMP | [1791-1809] | [1762-1780] |
| 53 | AGCGCUACGAUGUGGAAAC | GUUUCCACAUCGUAGCGCU | CHMP | [1789-1807] | [1760-1778] |
| 54 | GUCCUGCACAACUGUAUCU | AGAUACAGUUGUGCAGGAC | CHMP | [1725-1743] | [1696-1714] |
| 55 | CAACAGUGUGGAGAGGUAU | AUACCUCUCCACACUGUUG | CHMP | [1496-1514] | [1467-1485] |
| 56 | UGCAUCAACUGGGUCAAGU | ACUUGACCCAGUUGAUGCA | CHMP | [930-948] | [901-919] |
| 57 | GAACGGUGCUGUCAUGUAC | GUACAUGACAGCACCGUUC | | [653-671] | [624-642] |
| 58 | UCCAAGGAAAAUAAAGAAC | GUUCUUUAUUUUCCUUGGA | CHMP | [2549-2567] | [2520-2538] |
| 59 | UUGAUAAGUAACCCUGUAA | UUACAGGGUUACUUAUCAA | CHMP | [2527-2545] | [2498-2516] |
| 60 | UGGACAGUUAUUUUGUUGA | UCAACAAAAUAACUGUCCA | CHMP | [2512-2530] | [2483-2501] |
| 61 | GUACAUAGAAGCCACCGGA | UCCGGUGGCUUCUAUGUAC | CHMP | [2475-2493] | [2446-2464] |
| 62 | UCAGUGUUAAAAUGACAUC | GAUGUCAUUUUAACACUGA | CHMP | [2190-2208] | [2161-2179] |
| 63 | CUCAGUGUUAAAAUGACAU | AUGUCAUUUUAACACUGAG | CHMP | [2189-2207] | [2160-2178] |
| 64 | CAAAAAUACAGUCCAAUGG | CCAUUGGACUGUAUUUUUG | | [2080-2098] | [2051-2069] |
| 65 | UCAAGUACGACUGCGAACA | UGUUCGCAGUCGUACUUGA | CHMP | [943-961] | [914-932] |
| 66 | AGAUUGGCUGUGUGGAGUU | AACUCCACACAGCCAAUCU | | [763-781] | [734-752] |
| 67 | UGAACGGUGCUGUCAUGUA | UACAUGACAGCACCGUUCA | | [652-670] | [623-641] |
| 68 | GGAGCGCCUCAUUGAAUUC | GAAUUCAAUGAGGCGCUCC | | [584-602] | [555-573] |
| 69 | GGUGUCCAUUGAGGGUAUC | GAUACCCUCAAUGGACACC | CHMP | [551-569] | [522-540] |
| 70 | AGUGUUAAAAUGACAUCUC | GAGAUGUCAUUUUAACACU | CHMP | [2192-2210] | [2163-2181] |
| 71 | CAGUGUUAAAAUGACAUCU | AGAUGUCAUUUUAACACUG | CHMP | [2191-2209] | [2162-2180] |
| 72 | CCUCAGUGUUAAAAUGACA | UGUCAUUUUAACACUGAGG | CHMP | [2188-2206] | [2159-2177] |
| 73 | UGUACAAAAACCGGGACUA | UAGUCCCGGUUUUUGUACA | CHMP | [2115-2133] | [2086-2104] |
| 74 | GCACUUUUGUUUCUUGGGC | GCCCAAGAAACAAAAGUGC | CHMP | [2062-2080] | [2033-2051] |
| 75 | GAGGCACUUUUGUUUCUUG | CAAGAAACAAAAGUGCCUC | CHMP | [2059-2077] | [2030-2048] |
| 76 | ACAGUGUGGAGUGUUACGA | UCGUAACACUCCACACUGU | CHMP | [1921-1939] | [1892-1910] |
| 77 | CCUUUGGCAUCAUGAACGA | UCGUUCAUGAUGCCAAAGG | CHMP | [373-391] | [344-362] |
| 78 | ACUUUUGUUUCUUGGGCAA | UUGCCCAAGAAACAAAAGU | CHMP | [2064-2082] | [2035-2053] |
| 79 | GCAGAUUGACCAGCAGAAC | GUUCUGCUGGUCAAUCUGC | CHMP | [2030-2048] | [2001-2019] |
| 80 | GGACAGUGUGGAGUGUUAC | GUAACACUCCACACUGUCC | CHMP | [1919-1937] | [1890-1908] |
| 81 | GGAAACAGAGACGUGGACU | AGUCCACGUCUCUGUUUCC | CHMP | [1802-1820] | [1773-1791] |
| 82 | UGGAAACAGAGACGUGGAC | GUCCACGUCUCUGUUUCCA | CHMP | [1801-1819] | [1772-1790] |
| 83 | GAUUUAUUCUUGGAUACCU | AGGUAUCCAAGAAUAAAUC | CHMP | [2389-2407] | [2360-2378] |
| 84 | UUUGUUUCUUGGGCAAAAA | UUUUUGCCCAAGAAACAAA | CHMP | [2067-2085] | [2038-2056] |
| 85 | CACUUUUGUUUCUUGGGCA | UGCCCAAGAAACAAAAGUG | CHMP | [2063-2081] | [2034-2052] |
| 86 | CUGGACAGUGUGGAGUGUU | AACACUCCACACUGUCCAG | CHMP | [1917-1935] | [1888-1906] |
| 87 | CCGGGAGUACAUCUACAUG | CAUGUAGAUGUACUCCCGG | CHMP | [794-812] | [765-783] |
| 88 | CCAAGGAAAAUAAAGAACA | UGUUCUUUAUUUUCCUUGG | CHMP | [2550-2568] | [2521-2539] |
| 89 | GGAAAAUAAAGAACAGACU | AGUCUGUUCUUUAUUUUCC | CHMP | [2554-2572] | [2525-2543] |
| 90 | UGGCGAAUGAUCACAGCAA | UUGCUGUGAUCAUUCGCCA | CHMP | [1674-1692] | [1645-1663] |
| 91 | CCGGAUGGACAGUUAUUUU | AAAAUAACUGUCCAUCCGG | CHMP | [2507-2525] | [2478-2496] |
| 92 | AGCAGGCCUUUGGCAUCAU | AUGAUGCCAAAGGCCUGCU | CHMP | [367-385] | [338-356] |
| 93 | GUGCUGUCAUGUACCAGAU | AUCUGGUACAUGACAGCAC | | [658-676] | [629-647] |
| 94 | CAAGGAAAAUAAAGAACAG | CUGUUCUUUAUUUUCCUUG | CHMP | [2551-2569] | [2522-2540] |
| 95 | GCUGUCACCAUGUGAUUUA | UAAAUCACAUGGUGACAGC | | [2376-2394] | [2347-2365] |
| 96 | CCGCCUUAAUUCAGCUGAG | CUCAGCUGAAUUAAGGCGG | CHMP | [1631-1649] | [1602-1620] |
| 97 | GGAGGAUCAUACCAAGCAG | CUGCUUGGUAUGAUCCUCC | CHMP | [353-371] | [324-342] |
| 98 | UGGUGUCCAUUGAGGGUAU | AUACCCUCAAUGGACACCA | CHMP | [550-568] | [521-539] |
| 99 | CGGAUGGACAGUUAUUUUG | CAAAAUAACUGUCCAUCCG | CHMP | [2508-2526] | [2479-2497] |
| 100 | GCCUCAGUGUUAAAAUGAC | GUCAUUUUAACACUGAGGC | CHMP | [2187-2205] | [2158-2176] |
| 101 | GCGUCCUGCACAACUGUAU | AUACAGUUGUGCAGGACGC | CHMP | [1723-1741] | [1694-1712] |
| 102 | CGAGUGGCGAAUGAUCACA | UGUGAUCAUUCGCCACUCG | CHMP | [1670-1688] | [1641-1659] |
| 103 | CCCUGGAGGAUCAUACCAA | UUGGUAUGAUCCUCCAGGG | CHMP | [349-367] | [320-338] |
| 104 | GGAUGGACAGUUAUUUUGU | ACAAAAUAACUGUCCAUCC | CHMP | [2509-2527] | [2480-2498] |
| 105 | GCAAUGAACACCAUCCGAA | UUCGGAUGGUGUUCAUUGC | CHMP | [1689-1707] | [1660-1678] |
| 106 | AGGAAAAUAAAGAACAGAC | GUCUGUUCUUUAUUUUCCU | CHMP | [2553-2571] | [2524-2542] |
| 107 | CCUGUAAUUUUCCAAGGAA | UUCCUUGGAAAAUUACAGG | CHMP | [2539-2557] | [2510-2528] |
| 108 | CCACCGGAUGGACAGUUAU | AUAACUGUCCAUCCGGUGG | CHMP | [2504-2522] | [2475-2493] |
| 109 | AGAAGUCCAAAGCGGGAAU | AUUCCCGCUUUGGACUUCU | CHMP | [2214-2232] | [2185-2203] |
| 110 | CGGGACAAACCGCCUUAAU | AUUAAGGCGGUUUGUCCCG | CHMP | [1622-1640] | [1593-1611] |
| 111 | ACACCCUGGAGGAUCAUAC | GUAUGAUCCUCCAGGGUGU | CHMP | [346-364] | [317-335] |
| 112 | CUACACCCUGGAGGAUCAU | AUGAUCCUCCAGGGUGUAG | CHMP | [344-362] | [315-333] |
| 113 | UGAGCAGAUUGGCUGUGUG | CACACAGCCAAUCUGCUCA | | [758-776] | [729-747] |
| 114 | GUACCAGAUCGACAGCGUU | AACGCUGUCGAUCUGGUAC | CHMP | [668-686] | [639-657] |
| 115 | GUGGUGUCCAUUGAGGGUA | UACCCUCAAUGGACACCAC | CHMP | [549-567] | [520-538] |
| 116 | CCCUGUAAUUUUCCAAGGA | UCCUUGGAAAAUUACAGGG | CHMP | [2538-2556] | [2509-2527] |
| 117 | UAACCCUGUAAUUUUCCAA | UUGGAAAAUUACAGGGUUA | CHMP | [2535-2553] | [2506-2524] |
| 118 | GUAACCCUGUAAUUUUCCA | UGGAAAAUUACAGGGUUAC | CHMP | [2534-2552] | [2505-2523] |
| 119 | UCAUACCAAGCAGGCCUUU | AAAGGCCUGCUUGGUAUGA | CHMP | [359-377] | [330-348] |
| 120 | CGCCUUAAUUCAGCUGAGU | ACUCAGCUGAAUUAAGGCG | CHMP | [1632-1650] | [1603-1621] |
| 121 | ACCGCCUUAAUUCAGCUGA | UCAGCUGAAUUAAGGCGGU | CHMP | [1630-1648] | [1601-1619] |
| 122 | UCGUCUCCUUUAUGCCGUG | CACGGCAUAAAGGAGACGA | | [1592-1610] | [1563-1581] |
| 123 | UGGAGGAUCAUACCAAGCA | UGCUUGGUAUGAUCCUCCA | CHMP | [352-370] | [323-341] |
| 124 | UCAUCGAUGGCCACAUCUA | UAGAUGUGGCCAUCGAUGA | CHMP | [1444-1462] | [1415-1433] |
| 125 | GCUACCUGGAGGCUUACAA | UUGUAAGCCUCCAGGUAGC | CHMP | [1204-1222] | [1175-1193] |
| 126 | AGCAAGAGGAGUUCUUCAA | UUGAAGAACUCCUCUUGCU | CHMP | [832-850] | [803-821] |
| 127 | AGUACAUCUACAUGCAUUU | AAAUGCAUGUAGAUGUACU | CHMP | [799-817] | [770-788] |
| 128 | CUGAGCAGAUUGGCUGUGU | ACACAGCCAAUCUGCUCAG | | [757-775] | [728-746] |
| 129 | CAGACUAACUAGUGUCUUU | AAAGACACUAGUUAGUCUG | CHMP | [2567-2585] | [2538-2556] |
| 130 | GGAGUGUUACGACCCAGAU | AUCUGGGUCGUAACACUCC | CHMP | [1928-1946] | [1899-1917] |
| 131 | UGGAGAGGUAUGAGCCAGA | UCUGGCUCAUACCUCUCCA | CHMP | [1504-1522] | [1475-1493] |
| 132 | CCAUGACCAAUCAGUGGUC | GACCACUGAUUGGUCAUGG | CHMP | [1378-1396] | [1349-1367] |
| 133 | AGCUGCAGAAGUGCGAGAU | AUCUCGCACUUCUGCAGCU | CHMP | [1036-1054] | [1007-1025] |
| 134 | AGAUGCAGCUGCAGAAGUG | CACUUCUGCAGCUGCAUCU | CHMP | [1030-1048] | [1001-1019] |
| 135 | CUGUAAUUUUCCAAGGAAA | UUUCCUUGGAAAAUUACAG | CHMP | [2540-2558] | [2511-2529] |
| 136 | CUGUCACCAUGUGAUUUAU | AUAAAUCACAUGGUGACAG | | [2377-2395] | [2348-2366] |
| 137 | CAGCUGUCACCAUGUGAUU | AAUCACAUGGUGACAGCUG | | [2374-2392] | [2345-2363] |
| 138 | GACCAGCAGAACUGUACCU | AGGUACAGUUCUGCUGGUC | CHMP | [2037-2055] | [2008-2026] |
| 139 | GGGAGAAUCUACGUCCUUG | CAAGGACGUAGAUUCUCCC | CHMP | [1875-1893] | [1846-1864] |
| 140 | ACGGGACAAACCGCCUUAA | UUAAGGCGGUUUGUCCCGU | CHMP | [1621-1639] | [1592-1610] |
| 141 | CCCAUGACCAAUCAGUGGU | ACCACUGAUUGGUCAUGGG | CHMP | [1377-1395] | [1348-1366] |
| 142 | AGGAGUUCUUCAACCUGUC | GACAGGUUGAAGAACUCCU | CHMP, Rat, Ms | [838-856] | [809-827] |
| 143 | GCAAGAGGAGUUCUUCAAC | GUUGAAGAACUCCUCUUGC | CHMP | [833-851] | [804-822] |
| 144 | CCAAGCAAGAGGAGUUCUU | AAGAACUCCUCUUGCUUGG | CHMP | [829-847] | [800-818] |
| 145 | GAAGUCCAAAGCGGGAAUC | GAUUCCCGCUUUGGACUUC | CHMP | [2215-2233] | [2186-2204] |
| 146 | GGCUAUGAUGGUCACACGU | ACGUGUGACCAUCAUAGCC | CHMP | [1896-1914] | [1867-1885] |
| 147 | GGAGAAUCUACGUCCUUGG | CCAAGGACGUAGAUUCUCC | CHMP | [1876-1894] | [1847-1865] |
| 148 | CGAACUUCCUGCAGAUGCA | UGCAUCUGCAGGAAGUUCG | CHMP | [1018-1036] | [989-1007] |
| 149 | CCUGCAUCAACUGGGUCAA | UUGACCCAGUUGAUGCAGG | CHMP | [928-946] | [899-917] |
| 150 | UGGCCAAGCAAGAGGAGUU | AACUCCUCUUGCUUGGCCA | CHMP | [826-844] | [797-815] |
| 151 | CUGUCAUGUACCAGAUCGA | UCGAUCUGGUACAUGACAG | CHMP | [661-679] | [632-650] |
| 152 | GGUGCUGUCAUGUACCAGA | UCUGGUACAUGACAGCACC | | [657-675] | [628-646] |
| 153 | ACCGGAUGGACAGUUAUUU | AAAUAACUGUCCAUCCGGU | CHMP | [2506-2524] | [2477-2495] |
| 154 | GCCUGUACAUAGAAGCCAC | GUGGCUUCUAUGUACAGGC | CHMP | [2471-2489] | [2442-2460] |
| 155 | UGGGCAAAAAUACAGUCCA | UGGACUGUAUUUUUGCCCA | | [2076-2094] | [2047-2065] |
| 156 | AGGCCUUUGGCAUCAUGAA | UUCAUGAUGCCAAAGGCCU | CHMP | [370-388] | [341-359] |
| 157 | UCCUCAAUCGUCUCCUUUA | UAAAGGAGACGAUUGAGGA | | [1585-1603] | [1556-1574] |
| 158 | CCUGGAGGAUCAUACCAAG | CUUGGUAUGAUCCUCCAGG | CHMP | [350-368] | [321-339] |
| 159 | GUACAUCUACAUGCAUUUU | AAAAUGCAUGUAGAUGUAC | CHMP | [800-818] | [771-789] |
| 160 | ACAGACUAACUAGUGUCUU | AAGACACUAGUUAGUCUGU | CHMP | [2566-2584] | [2537-2555] |
| 161 | GGCCUGUACAUAGAAGCCA | UGGCUUCUAUGUACAGGCC | CHMP | [2470-2488] | [2441-2459] |
| 162 | CUGUACCUGUUGAGGCACU | AGUGCCUCAACAGGUACAG | CHMP | [2048-2066] | [2019-2037] |
| 163 | CCAGAUACAGACACCUGGA | UCCAGGUGUCUGUAUCUGG | CHMP | [1941-1959] | [1912-1930] |
| 164 | CGUCCUUGGAGGCUAUGAU | AUCAUAGCCUCCAAGGACG | CHMP | [1886-1904] | [1857-1875] |
| 165 | CACAACUGUAUCUAUGCUG | CAGCAUAGAUACAGUUGUG | CHMP | [1731-1749] | [1702-1720] |
| 166 | CCAACUUCGCUGAGCAGAU | AUCUGCUCAGCGAAGUUGG | CHMP | [748-766] | [719-737] |
| 167 | ACGGUGCUGUCAUGUACCA | UGGUACAUGACAGCACCGU | | [655-673] | [626-644] |
| 168 | AGUGUGUCCUCCACGUCAU | AUGACGUGGAGGACACACU | CHMP | [634-652] | [605-623] |
| 169 | AGAAGUGUGUCCUCCACGU | ACGUGGAGGACACACUUCU | CHMP | [631-649] | [602-620] |
| 170 | UGGAGGUGGUGUCCAUUGA | UCAAUGGACACCACCUCCA | CHMP, Rat | [544-562] | [515-533] |
| 171 | AGACUAACUAGUGUCUUUC | GAAAGACACUAGUUAGUCU | CHMP | [2568-2586] | [2539-2557] |
| 172 | GAACAGACUAACUAGUGUC | GACACUAGUUAGUCUGUUC | CHMP | [2564-2582] | [2535-2553] |
| 173 | CAGGUCAAGUACCAGGAUG | CAUCCUGGUACUUGACCUG | CHMP | [429-447] | [400-418] |
| 174 | CAGAACUGUACCUGUUGAG | CUCAACAGGUACAGUUCUG | CHMP | [2043-2061] | [2014-2032] |
| 175 | CCAGCAGAACUGUACCUGU | ACAGGUACAGUUCUGCUGG | CHMP | [2039-2057] | [2010-2028] |
| 176 | GCCUUAAUUCAGCUGAGUG | CACUCAGCUGAAUUAAGGC | CHMP | [1633-1651] | [1604-1622] |
| 177 | GUCCUCAAUCGUCUCCUUU | AAAGGAGACGAUUGAGGAC | | [1584-1602] | [1555-1573] |
| 178 | UGUCCUCAAUCGUCUCCUU | AAGGAGACGAUUGAGGACA | | [1583-1601] | [1554-1572] |
| 179 | GAGGAGUUCUUCAACCUGU | ACAGGUUGAAGAACUCCUC | CHMP, Rat, Ms | [837-855] | [808-826] |
| 180 | GACUAACUAGUGUCUUUCA | UGAAAGACACUAGUUAGUC | CHMP | [2569-2587] | [2540-2558] |
| 181 | CACCGGAUGGACAGUUAUU | AAUAACUGUCCAUCCGGUG | CHMP | [2505-2523] | [2476-2494] |
| 182 | GUCACCAUGUGAUUUAUUC | GAAUAAAUCACAUGGUGAC | | [2379-2397] | [2350-2368] |
| 183 | ACAUCUCAAAAGAAGUCCA | UGGACUUCUUUUGAGAUGU | CHMP | [2204-2222] | [2175-2193] |
| 184 | UGACAUCUCAAAAGAAGUC | GACUUCUUUUGAGAUGUCA | CHMP | [2202-2220] | [2173-2191] |
| 185 | CUGUUGAGGCACUUUUGUU | AACAAAAGUGCCUCAACAG | CHMP | [2054-2072] | [2025-2043] |
| 186 | GUACCUGUUGAGGCACUUU | AAAGUGCCUCAACAGGUAC | CHMP | [2050-2068] | [2021-2039] |
| 187 | UGUACCUGUUGAGGCACUU | AAGUGCCUCAACAGGUACA | CHMP | [2049-2067] | [2020-2038] |
| 188 | GAAGCAGAUUGACCAGCAG | CUGCUGGUCAAUCUGCUUC | CHMP | [2027-2045] | [1998-2016] |
| 189 | CAGAUACAGACACCUGGAG | CUCCAGGUGUCUGUAUCUG | CHMP | [1942-1960] | [1913-1931] |
| 190 | UGGAGUGUUACGACCCAGA | UCUGGGUCGUAACACUCCA | CHMP | [1927-1945] | [1898-1916] |
| 191 | ACACGUUCCUGGACAGUGU | ACACUGUCCAGGAACGUGU | CHMP | [1909-1927] | [1880-1898] |
| 192 | GGAGGCUAUGAUGGUCACA | UGUGACCAUCAUAGCCUCC | CHMP | [1893-1911] | [1864-1882] |
| 193 | CUGAGUGUUACUACCCAGA | UCUGGGUAGUAACACUCAG | CHMP | [1645-1663] | [1616-1634] |
| 194 | GCUGAGUGUUACUACCCAG | CUGGGUAGUAACACUCAGC | CHMP | [1644-1662] | [1615-1633] |
| 195 | CCUGUCUUCAAGGCCAUGU | ACAUGGCCUUGAAGACAGG | CHMP | [498-516] | [469-487] |
| 196 | ACCCUGUAAUUUUCCAAGG | CCUUGGAAAAUUACAGGGU | CHMP | [2537-2555] | [2508-2526] |
| 197 | UGUCACCAUGUGAUUUAUU | AAUAAAUCACAUGGUGACA | | [2378-2396] | [2349-2367] |
| 198 | UACCUGUUGAGGCACUUUU | AAAAGUGCCUCAACAGGUA | CHMP | [2051-2069] | [2022-2040] |
| 199 | AGAUUGACCAGCAGAACUG | CAGUUCUGCUGGUCAAUCU | CHMP | [2032-2050] | [2003-2021] |
| 200 | AGCAGAUUGACCAGCAGAA | UUCUGCUGGUCAAUCUGCU | CHMP | [2029-2047] | [2000-2018] |
| 201 | GGAAGCAGAUUGACCAGCA | UGCUGGUCAAUCUGCUUCC | CHMP | [2026-2044] | [1997-2015] |
| 202 | GAGGCUAUGAUGGUCACAC | GUGUGACCAUCAUAGCCUC | CHMP | [1894-1912] | [1865-1883] |
| 203 | AGCUGAGUGUUACUACCCA | UGGGUAGUAACACUCAGCU | CHMP | [1643-1661] | [1614-1632] |
| 204 | GGUCAAGUACGACUGCGAA | UUCGCAGUCGUACUUGACC | CHMP | [941-959] | [912-930] |
| 205 | ACAUCUACAUGCAUUUUGG | CCAAAAUGCAUGUAGAUGU | CHMP | [802-820] | [773-791] |
| 206 | GUGUCCUCCACGUCAUGAA | UUCAUGACGUGGAGGACAC | CHMP | [637-655] | [608-626] |
| 207 | GCCUUUGGCAUCAUGAACG | CGUUCAUGAUGCCAAAGGC | CHMP | [372-390] | [343-361] |
| 208 | UGGAGGCUAUGAUGGUCAC | GUGACCAUCAUAGCCUCCA | CHMP | [1892-1910] | [1863-1881] |
| 209 | GGCCUUUGGCAUCAUGAAC | GUUCAUGAUGCCAAAGGCC | CHMP | [371-389] | [342-360] |
| 210 | UGCAAGGACUACCUGGUCA | UGACCAGGUAGUCCUUGCA | CHMP | [1074-1092] | [1045-1063] |
| 211 | CGGGAGUACAUCUACAUGC | GCAUGUAGAUGUACUCCCG | CHMP | [795-813] | [766-784] |
| 212 | UCAUGAACGGUGCUGUCAU | AUGACAGCACCGUUCAUGA | | [649-667] | [620-638] |
| 213 | CCCUCUGGAAAUGUGGUUC | GAACCACAUUUCCAGAGGG | CHMP | [2442-2460] | [2413-2431] |
| 214 | AGCUGUGUGACGUCACACU | AGUGUGACGUCACACAGCU | CHMP | [409-427] | [380-398] |
| 215 | CUAUGAUGGUCACACGUUC | GAACGUGUGACCAUCAUAG | CHMP | [1898-1916] | [1869-1887] |
| 216 | GAAAUGUGGUUCCCAGGGA | UCCCUGGGAACCACAUUUC | CHMP | [2449-2467] | [2420-2438] |
| 217 | CUGGAAAUGUGGUUCCCAG | CUGGGAACCACAUUUCCAG | CHMP | [2446-2464] | [2417-2435] |
| 218 | CUAUGAUGGUCAGGACCAG | CUGGUCCUGACCAUCAUAG | CHMP | [1757-1775] | [1728-1746] |
| 219 | CACUGCAGGUCAAGUACCA | UGGUACUUGACCUGCAGUG | CHMP | [424-442] | [395-413] |
| 220 | UGUUGAGGCACUUUUGUUU | AAACAAAAGUGCCUCAACA | CHMP | [2055-2073] | [2026-2044] |
| 221 | AGAGACGUGGACUUUCGUA | UACGAAAGUCCACGUCUCU | CHMP | [1808-1826] | [1779-1797] |
| 222 | GCCAAGCAAGAGGAGUUCU | AGAACUCCUCUUGCUUGGC | CHMP | [828-846] | [799-817] |
| 223 | AAAUGUGGUUCCCAGGGAU | AUCCCUGGGAACCACAUUU | CHMP | [2450-2468] | [2421-2439] |
| 224 | UGGCUGUCCUCAAUCGUCU | AGACGAUUGAGGACAGCCA | CHMP | [1579-1597] | [1550-1568] |
| 225 | UGCAGAUGCAGCUGCAGAA | UUCUGCAGCUGCAUCUGCA | CHMP | [1027-1045] | [998-1016] |

TABLE B6
KEAP1 - 19-mer oligomer pairs kelch-like ECH-associated

| | | | | Human- | |
|---|---|---|---|---|---|
| | | | | 45269144: | Human- |
| No. | Sense | AntiSense | Other Sp | 186-2060 | 45269143: 157-2031 |

| | | | | | |
|---|---|---|---|---|---|
| 1 | GGAUGGGCCUGUACAUAGA | UCUAUGUACAGGCCCAUCC | Chmp | [2465-2483] | [2436-2454] |
| 2 | CAUGGAGCGCCUCAUUGAA | UUCAAUGAGGCGCUCCAUG | | [581-599] | [552-570] |
| 3 | CAUUGUUUUUGUACAAAAA | UUUUUGUACAAAAACAAUG | Chmp | [2106-2124] | [2077-2095] |
| 4 | GCUACGAUGUGGAAACAGA | UCUGUUUCCACAUCGUAGC | Chmp | [1792-1810] | [1763-1781] |
| 5 | GAAUGAUCACAGCAAUGAA | UUCAUUGCUGUGAUCAUUC | Chmp | [1678-1696] | [1649-1667] |
| 6 | GCAUCAACUGGGUCAAGUA | UACUUGACCCAGUUGAUGC | GP, Chn, Chmp | [931-949] | [902-920] |
| 7 | AAAUGACAUCUCAAAAGAA | UUCUUUUGAGAUGUCAUUU | Chmp | [2199-2217] | [2170-2188] |
| 8 | CAUCCAGCCCUGUCUUCAA | UUGAAGACAGGGCUGGAUG | Chmp | [490-508] | [461-479] |
| 9 | GGCGAAUGAUCACAGCAAU | AUUGCUGUGAUCAUUCGCC | Chmp | [1675-1693] | [1646-1664] |
| 10 | GAAAAGACAGCACUGCAAA | UUUGCAGUGCUGUCUUUUC | Chmp | [2137-2155] | [2108-2126] |
| 11 | CCAGGAUGCCUCAGUGUUA | UAACACUGAGGCAUCCUGG | Chmp | [2180-2198] | [2151-2169] |
| 12 | UGUUAAAAUGACAUCUCAA | UUGAGAUGUCAUUUUAACA | Chmp | [2194-2212] | [2165-2183] |
| 13 | UGGACAGUGUGGAGUGUUA | UAACACUCCACACUGUCCA | Chmp | [1918-1936] | [1889-1907] |
| 14 | GAGUAUCAUUGUUUUUGUA | UACAAAAACAAUGAUACUC | Chmp | [2100-2118] | [2071-2089] |
| 15 | GAUGGGCCUGUACAUAGAA | UUCUAUGUACAGGCCCAUC | Chmp | [2466-2484] | [2437-2455] |
| 16 | AAACCGGGACUAAAAGAAA | UUUCUUUUAGUCCCGGUUU | Chmp | [2122-2140] | [2093-2111] |
| 17 | GUGUUACGACCCAGAUACA | UGUAUCUGGGUCGUAACAC | Chmp | [1931-1949] | [1902-1920] |
| 18 | GCAAGGACUACCUGGUCAA | UUGACCAGGUAGUCCUUGC | Chmp | [1075-1093] | [1046-1064] |
| 19 | CAUCGAUGGCCACAUCUAU | AUAGAUGUGGCCAUCGAUG | Chmp | [1445-1463] | [1416-1434] |
| 20 | ACAGUUAUUUUGUUGAUAA | UUAUCAACAAAAUAACUGU | Chmp | [2515-2533] | [2486-2504] |
| 21 | ACAACAGUGUGGAGAGGUA | UACCUCUCCACACUGUUGU | Chmp | [1495-1513] | [1466-1484] |
| 22 | GAGUGUUACGACCCAGAUA | UAUCUGGGUCGUAACACUC | Chmp | [1929-1947] | [1900-1918] |
| 23 | GUUACGACCCAGAUACAGA | UCUGUAUCUGGGUCGUAAC | Chmp | [1933-1951] | [1904-1922] |
| 24 | GGACAGUUAUUUUGUUGAU | AUCAACAAAAUAACUGUCC | Chmp | [2513-2531] | [2484-2502] |
| 25 | GAAAAUAAAGAACAGACUA | UAGUCUGUUCUUUAUUUUC | Chmp | [2555-2573] | [2526-2544] |
| 26 | GGGACAAACCGCCUUAAUU | AAUUAAGGCGGUUUGUCCC | Chmp | [1623-1641] | [1594-1612] |
| 27 | AGGAACGAGUGGCGAAUGA | UCAUUCGCCACUCGUUCCU | Chmp | [1665-1683] | [1636-1654] |
| 28 | UCAAGGCCAUGUUCACCAA | UUGGUGAACAUGGCCUUGA | GP, Chn, Chmp | [505-523] | [476-494] |
| 29 | AAGACAGCACUGCAAAUAA | UUAUUUGCAGUGCUGUCUU | Chmp | [2140-2158] | [2111-2129] |
| 30 | GAGUGUUACUACCCAGAGA | UCUCUGGGUAGUAACACUC | Chmp | [1647-1665] | [1618-1636] |
| 31 | GUAUCAUUGUUUUUGUACA | UGUACAAAAACAAUGAUAC | Chmp | [2102-2120] | [2073-2091] |
| 32 | UUAAAAUGACAUCUCAAAA | UUUUGAGAUGUCAUUUUAA | Chmp | [2196-2214] | [2167-2185] |
| 33 | CUACGAUGUGGAAACAGAG | CUCUGUUUCCACAUCGUAG | Chmp | [1793-1811] | [1764-1782] |
| 34 | UCAACUGGGUCAAGUACGA | UCGUACUUGACCCAGUUGA | GP, Chn, Chmp | [934-952] | [905-923] |
| 35 | GUGGAAACAGAGACGUGGA | UCCACGUCUCUGUUUCCAC | Chmp | [1800-1818] | [1771-1789] |
| 36 | GGGAGUACAUCUACAUGCA | UGCAUGUAGAUGUACUCCC | Chmp | [796-814] | [767-785] |
| 37 | CAGGAUGCCUCAGUGUUAA | UUAACACUGAGGCAUCCUG | Chmp | [2181-2199] | [2152-2170] |
| 38 | UCAUUGUUUUUGUACAAAA | UUUUGUACAAAAACAAUGA | Chmp | [2105-2123] | [2076-2094] |
| 39 | UACAAAAACCGGGACUAAA | UUUAGUCCCGGUUUUUGUA | Chmp | [2117-2135] | [2088-2106] |
| 40 | CAGUUAUUUUGUUGAUAAG | CUUAUCAACAAAAUAACUG | Chmp | [2516-2534] | [2487-2505] |
| 41 | GUUAAAAUGACAUCUCAAA | UUUGAGAUGUCAUUUUAAC | Chmp | [2195-2213] | [2166-2184] |
| 42 | GAUGCCUCAGUGUUAAAAU | AUUUUAACACUGAGGCAUC | Chmp | [2184-2202] | [2155-2173] |
| 43 | GAGCGCUACGAUGUGGAAA | UUUCCACAUCGUAGCGCUC | Chmp | [1788-1806] | [1759-1777] |
| 44 | CUACGUCCUUGGAGGCUAU | AUAGCCUCCAAGGACGUAG | Chmp | [1883-1901] | [1854-1872] |
| 45 | UGGUCAAGAUCUUCGAGGA | UCCUCGAAGAUCUUGACCA | Chmp | [1087-1105] | [1058-1076] |
| 46 | GUUGAUAAGUAACCCUGUA | UACAGGGUUACUUAUCAAC | Chmp | [2526-2544] | [2497-2515] |
| 47 | GCGCUACGAUGUGGAAACA | UGUUUCCACAUCGUAGCGC | Chmp | [1790-1808] | [1761-1779] |
| 48 | UCAUGGAGCGCCUCAUUGA | UCAAUGAGGCGCUCCAUGA | | [580-598] | [551-569] |
| 49 | AAAAUGACAUCUCAAAAGA | UCUUUUGAGAUGUCAUUUU | Chmp | [2198-2216] | [2169-2187] |
| 50 | CAGUGUGGAGUGUUACGAC | GUCGUAACACUCCACACUG | Chmp | [1922-1940] | [1893-1911] |
| 51 | ACCUGGUCAAGAUCUUCGA | UCGAAGAUCUUGACCAGGU | Chmp | [1084-1102] | [1055-1073] |
| 52 | GAGAAUCUACGUCCUUGGA | UCCAAGGACGUAGAUUCUC | Chmp | [1877-1895] | [1848-1866] |
| 53 | AACGAGUGGCGAAUGAUCA | UGAUCAUUCGCCACUCGUU | Chmp | [1668-1686] | [1639-1657] |
| 54 | GACAGUUAUUUUGUUGAUA | UAUCAACAAAAUAACUGUC | Chmp | [2514-2532] | [2485-2503] |
| 55 | AAAAUAAAGAACAGACUAA | UUAGUCUGUUCUUUAUUUU | Chmp | [2556-2574] | [2527-2545] |
| 56 | UGUAAUUUUCCAAGGAAAA | UUUUCCUUGGAAAAUUACA | Chmp | [2541-2559] | [2512-2530] |
| 57 | GAACGAGUGGCGAAUGAUC | GAUCAUUCGCCACUCGUUC | Chmp | [1667-1685] | [1638-1656] |
| 58 | UCAGCUACCUGGAGGCUUA | UAAGCCUCCAGGUAGCUGA | Chmp | [1201-1219] | [1172-1190] |
| 59 | GAUGGACAGUUAUUUUGUU | AACAAAAUAACUGUCCAUC | Chmp | [2510-2528] | [2481-2499] |
| 60 | UGUGAUUUAUUCUUGGAUA | UAUCCAAGAAUAAAUCACA | Chmp | [2386-2404] | [2357-2375] |
| 61 | CAGGCCUUUGGCAUCAUGA | UCAUGAUGCCAAAGGCCUG | Chmp | [369-387] | [340-358] |
| 62 | GUUAUUUUGUUGAUAAGUA | UACUUAUCAACAAAAUAAC | Chmp | [2518-2536] | [2489-2507] |
| 63 | AAAAGACAGCACUGCAAAU | AUUUGCAGUGCUGUCUUUU | Chmp | [2138-2156] | [2109-2127] |
| 64 | CAAAAACCGGGACUAAAAG | CUUUUAGUCCCGGUUUUUG | Chmp | [2119-2137] | [2090-2108] |
| 65 | AUCUCAAAAGAAGUCCAAA | UUUGGACUUCUUUUGAGAU | Chmp | [2206-2224] | [2177-2195] |
| 66 | GAGGUGGUGGUGUUGCUUA | UAAGCAACACCACCACCUC | | [153-171] | [124-142] |
| 67 | GCAGGUCAAGUACCAGGAU | AUCCUGGUACUUGACCUGC | Chmp | [428-446] | [399-417] |
| 68 | GAUAAGUAACCCUGUAAUU | AAUUACAGGGUUACUUAUC | Chmp | [2529-2547] | [2500-2518] |
| 69 | UGUUUCUUGGGCAAAAAUA | UAUUUUUGCCCAAGAAACA | Chmp | [2069-2087] | [2040-2058] |
| 70 | GUUACUACCCAGAGAGGAA | UUCCUCUCUGGGUAGUAAC | Chmp | [1651-1669] | [1622-1640] |
| 71 | CAAUGAACACCAUCCGAAG | CUUCGGAUGGUGUUCAUUG | Chmp | [1690-1708] | [1661-1679] |
| 72 | GAAACAGAGACGUGGACUU | AAGUCCACGUCUCUGUUUC | Chmp | [1803-1821] | [1774-1792] |
| 73 | UACGAUGUGGAAACAGAGA | UCUCUGUUUCCACAUCGUA | Chmp | [1794-1812] | [1765-1783] |
| 74 | CAAUGCUGACACGAAGGAU | AUCCUUCGUGUCAGCAUUG | Chmp | [1549-1567] | [1520-1538] |
| 75 | CCUUGGAGGCUAUGAUGGU | ACCAUCAUAGCCUCCAAGG | Chmp | [1889-1907] | [1860-1878] |
| 76 | CAGUGUGGAGAGGUAUGAG | CUCAUACCUCUCCACACUG | Chmp | [1499-1517] | [1470-1488] |
| 77 | UGUGGAGUGUUACGACCCA | UGGGUCGUAACACUCCACA | Chmp | [1925-1943] | [1896-1914] |
| 78 | UUUUGUUUCUUGGGCAAAA | UUUUGCCCAAGAAACAAAA | Chmp | [2066-2084] | [2037-2055] |
| 79 | GCAGAACUGUACCUGUUGA | UCAACAGGUACAGUUCUGC | Chmp | [2042-2060] | [2013-2031] |
| 80 | AAAACCGGGACUAAAAGAA | UUCUUUUAGUCCCGGUUUU | Chmp | [2121-2139] | [2092-2110] |
| 81 | GACAAACCGCCUUAAUUCA | UGAAUUAAGGCGGUUUGUC | Chmp | [1625-1643] | [1596-1614] |
| 82 | GAGGUGACCCGAAUGACAU | AUGUCAUUCGGGUCACCUC | Chmp | [1962-1980] | [1933-1951] |
| 83 | GGAGUACAUCUACAUGCAU | AUGCAUGUAGAUGUACUCC | Chmp | [797-815] | [768-786] |
| 84 | AAGCAAGAGGAGUUCUUCA | UGAAGAACUCCUCUUGCUU | Chmp | [831-849] | [802-820] |
| 85 | AGGACUACCUGGUCAAGAU | AUCUUGACCAGGUAGUCCU | Chmp | [1078-1096] | [1049-1067] |
| 86 | AAGUGUGUCCUCCACGUCA | UGACGUGGAGGACACACUU | Chmp | [633-651] | [604-622] |
| 87 | CAUGUGAUUUAUUCUUGGA | UCCAAGAAUAAAUCACAUG | | [2384-2402] | [2355-2373] |
| 88 | GGUCAAGUACCAGGAUGCA | UGCAUCCUGGUACUUGACC | Chmp | [431-449] | [402-420] |
| 89 | GAGUACAUCUACAUGCAUU | AAUGCAUGUAGAUGUACUC | Chmp | [798-816] | [769-787] |
| 90 | CAGCAGAACUGUACCUGUU | AACAGGUACAGUUCUGCUG | Chmp | [2040-2058] | [2011-2029] |
| 91 | AAGAAAAGACAGCACUGCA | UGCAGUGCUGUCUUUUCUU | Chmp | [2135-2153] | [2106-2124] |
| 92 | GUCAUGUACCAGAUCGACA | UGUCGAUCUGGUACAUGAC | Chn, Chmp | [663-681] | [634-652] |
| 93 | CAGAUUGACCAGCAGAACU | AGUUCUGCUGGUCAAUCUG | Chmp | [2031-2049] | [2002-2020] |
| 94 | AAUUUUCCAAGGAAAAUAA | UUAUUUUCCUUGGAAAAUU | Chmp | [2544-2562] | [2515-2533] |
| 95 | CCUGUUGAGGCACUUUUGU | ACAAAAGUGCCUCAACAGG | Chmp | [2053-2071] | [2024-2042] |
| 96 | CAACUUCGCUGAGCAGAUU | AAUCUGCUCAGCGAAGUUG | Chmp | [749-767] | [720-738] |
| 97 | CAACUGGUGACCCUCAUCA | UGAUGAGGGUCACCAGUUG | Chmp | [864-882] | [835-853] |
| 98 | UGAUGUACGCCUCCACUGA | UCAGUGGAGGCGUACAUCA | Chmp | [277-295] | [248-266] |
| 99 | UCAAGAUCUUCGAGGAGCU | AGCUCCUCGAAGAUCUUGA | Chmp | [1090-1108] | [1061-1079] |
| 100 | AAAAACCGGGACUAAAAGA | UCUUUUAGUCCCGGUUUUU | Chmp | [2120-2138] | [2091-2109] |
| 101 | UGUUGAUAAGUAACCCUGU | ACAGGGUUACUUAUCAACA | Chmp | [2525-2543] | [2496-2514] |
| 102 | UUGGCUGUGUGGAGUUGCA | UGCAACUCCACACAGCCAA | | [766-784] | [737-755] |
| 103 | UAAAGAACAGACUAACUAG | CUAGUUAGUCUGUUCUUUA | Chmp | [2560-2578] | [2531-2549] |
| 104 | AGCAAUGAACACCAUCCGA | UCGGAUGGUGUUCAUUGCU | Chmp | [1688-1706] | [1659-1677] |
| 105 | GUGUGGAAAGAGCAGGCUU | AAGCCUGCUCUUUCCACAC | Chmp | [2290-2308] | [2261-2279] |
| 106 | GUGGCGAAUGAUCACAGCA | UGCUGUGAUCAUUCGCCAC | Chmp | [1673-1691] | [1644-1662] |
| 107 | AGACAGCACUGCAAAUAAC | GUUAUUUGCAGUGCUGUCU | Chmp | [2141-2159] | [2112-2130] |
| 108 | AGAAGUGCGAGAUCCUGCA | UGCAGGAUCUCGCACUUCU | GP, Chn, Chmp | [1042-1060] | [1013-1031] |
| 109 | AAAGAGCAGGCUUCCAGGA | UCCUGGAAGCCUGCUCUUU | Chmp | [2296-2314] | [2267-2285] |
| 110 | UAUCAUUGUUUUUGUACAA | UUGUACAAAAACAAUGAUA | Chmp | [2103-2121] | [2074-2092] |
| 111 | CAGCACUGCAAAUAACCCA | UGGGUUAUUUGCAGUGCUG | Chmp | [2144-2162] | [2115-2133] |
| 112 | AGGGAUGGGCCUGUACAUA | UAUGUACAGGCCCAUCCCU | Chmp | [2463-2481] | [2434-2452] |
| 113 | AAUGACAUCUCAAAAGAAG | CUUCUUUUGAGAUGUCAUU | Chmp | [2200-2218] | [2171-2189] |
| 114 | CUACCUGGAGGCUUACAAC | GUUGUAAGCCUCCAGGUAG | Chmp | [1205-1223] | [1176-1194] |
| 115 | AACUGUACCUGUUGAGGCA | UGCCUCAACAGGUACAGUU | Chmp | [2046-2064] | [2017-2035] |
| 116 | GUUUCUUGGGCAAAAAUAC | GUAUUUUUGCCCAAGAAAC | | [2070-2088] | [2041-2059] |
| 117 | AAGGAAAAUAAAGAACAGA | UCUGUUCUUUAUUUUCCUU | Chmp | [2552-2570] | [2523-2541] |
| 118 | CUUUUGUUUCUUGGGCAAA | UUUGCCCAAGAAACAAAAG | Chmp | [2065-2083] | [2036-2054] |
| 119 | AAAGAACAGACUAACUAGU | ACUAGUUAGUCUGUUCUUU | Chmp | [2561-2579] | [2532-2550] |
| 120 | GAGUGGCGAAUGAUCACAG | CUGUGAUCAUUCGCCACUC | Chmp | [1671-1689] | [1642-1660] |
| 121 | AAUUCAGCUGAGUGUUACU | AGUAACACUCAGCUGAAUU | Chmp | [1638-1656] | [1609-1627] |
| 122 | UGUGGAGAGGUAUGAGCCA | UGGCUCAUACCUCUCCACA | Chmp | [1502-1520] | [1473-1491] |
| 123 | AUUUUCCAAGGAAAAUAAA | UUUAUUUUCCUUGGAAAAU | Chmp | [2545-2563] | [2516-2534] |
| 124 | GGUGGUGUUGCUUAUCUUC | GAAGAUAAGCAACACCACC | | [158-176] | [129-147] |
| 125 | AGUCCAAAGCGGGAAUCAU | AUGAUUCCCGCUUUGGACU | Chmp | [2217-2235] | [2188-2206] |
| 126 | AUAAAGAACAGACUAACUA | UAGUUAGUCUGUUCUUUAU | Chmp | [2559-2577] | [2530-2548] |
| 127 | ACUACCUGGUCAAGAUCUU | AAGAUCUUGACCAGGUAGU | Chmp | [1081-1099] | [1052-1070] |
| 128 | UUUUGUACAAAAACCGGGA | UCCCGGUUUUUGUACAAAA | Chmp | [2112-2130] | [2083-2101] |
| 129 | UACAUAGAAGCCACCGGAU | AUCCGGUGGCUUCUAUGUA | Chmp | [2476-2494] | [2447-2465] |
| 130 | CAGAUUGGCUGUGUGGAGU | ACUCCACACAGCCAAUCUG | | [762-780] | [733-751] |
| 131 | UAUUCUUGGAUACCUGGGA | UCCCAGGUAUCCAAGAAUA | Chmp | [2393-2411] | [2364-2382] |
| 132 | CAUGGAGGUGGUGUCCAUU | AAUGGACACCACCUCCAUG | Rat, Chmp | [542-560] | [513-531] |
| 133 | CAGAGACGUGGACUUUCGU | ACGAAAGUCCACGUCUCUG | Chmp | [1807-1825] | [1778-1796] |
| 134 | GAUUGACCAGCAGAACUGU | ACAGUUCUGCUGGUCAAUC | Chmp | [2033-2051] | [2004-2022] |
| 135 | UUUCUUGGGCAAAAAUACA | UGUAUUUUUGCCCAAGAAA | | [2071-2089] | [2042-2060] |
| 136 | GACAGUGUGGAGUGUUACG | CGUAACACUCCACACUGUC | Chmp | [1920-1938] | [1891-1909] |
| 137 | CAGAUGCAGCUGCAGAAGU | ACUUCUGCAGCUGCAUCUG | GP, Chn, Chmp | [1029-1047] | [1000-1018] |
| 138 | AAGCAGAUUGACCAGCAGA | UCUGCUGGUCAAUCUGCUU | Chmp | [2028-2046] | [1999-2017] |
| 139 | AGGCUAUGAUGGUCACACG | CGUGUGACCAUCAUAGCCU | Chmp | [1895-1913] | [1866-1884] |
| 140 | UUCCAAGGAAAAUAAAGAA | UUCUUUAUUUUCCUUGGAA | Chmp | [2548-2566] | [2519-2537] |
| 141 | AACCGCACCUUCAGCUACA | UGUAGCUGAAGGUGCGGUU | GP, Chmp | [330-348] | [301-319] |
| 142 | GUCUUCAAGGCCAUGUUCA | UGAACAUGGCCUUGAAGAC | Chmp | [501-519] | [472-490] |
| 143 | CGAAUGAUCACAGCAAUGA | UCAUUGCUGUGAUCAUUCG | Chmp | [1677-1695] | [1648-1666] |
| 144 | UGUCCAUUGAGGGUAUCCA | UGGAUACCCUCAAUGGACA | Chmp | [553-571] | [524-542] |
| 145 | UGGAGCGCCUCAUUGAAUU | AAUUCAAUGAGGCGCUCCA | | [583-601] | [554-572] |
| 146 | AAUAAAGAACAGACUAACU | AGUUAGUCUGUUCUUUAUU | Chmp | [2558-2576] | [2529-2547] |
| 147 | AUUGACCAGCAGAACUGUA | UACAGUUCUGCUGGUCAAU | Chmp | [2034-2052] | [2005-2023] |
| 148 | AAAGAAGUCCAAAGCGGGA | UCCCGCUUUGGACUUCUUU | Chmp | [2212-2230] | [2183-2201] |
| 149 | UUACGACCCAGAUACAGAC | GUCUGUAUCUGGGUCGUAA | Chmp | [1934-1952] | [1905-1923] |
| 150 | AGUUCUUCAACCUGUCCCA | UGGGACAGGUUGAAGAACU | GP, Chmp | [841-859] | [812-830] |
| 151 | UUAAUUCAGCUGAGUGUUA | UAACACUCAGCUGAAUUAA | Chmp | [1636-1654] | [1607-1625] |
| 152 | CAUUGAAUUCGCCUACACG | CGUGUAGGCGAAUUCAAUG | | [593-611] | [564-582] |
| 153 | AGAGGUGGUGGUGUUGCUU | AAGCAACACCACCACCUCU | | [152-170] | [123-141] |
| 154 | UAAGUAACCCUGUAAUUUU | AAAAUUACAGGGUUACUUA | Chmp | [2531-2549] | [2502-2520] |
| 155 | CAUCAACUGGGUCAAGUAC | GUACUUGACCCAGUUGAUG | GP, Chn, Chmp | [932-950] | [903-921] |
| 156 | GGUCAAGAUCUUCGAGGAG | CUCCUCGAAGAUCUUGACC | Chmp | [1088-1106] | [1059-1077] |
| 157 | UGCCUCAGUGUUAAAAUGA | UCAUUUUAACACUGAGGCA | Chmp | [2186-2204] | [2157-2175] |
| 158 | UCACAGCAAUGAACACCAU | AUGGUGUUCAUUGCUGUGA | Chmp | [1684-1702] | [1655-1673] |
| 159 | CAAGGACUACCUGGUCAAG | CUUGACCAGGUAGUCCUUG | Chmp | [1076-1094] | [1047-1065] |
| 160 | AGUUCAUGGCCCACAAGGU | ACCUUGUGGGCCAUGAACU | Chmp | [460-478] | [431-449] |
| 161 | UCUUCAAGGCCAUGUUCAC | GUGAACAUGGCCUUGAAGA | Chmp | [502-520] | [473-491] |
| 162 | UGCAGGUCAAGUACCAGGA | UCCUGGUACUUGACCUGCA | Chmp | [427-445] | [398-416] |
| 163 | GGUGUUGCUUAUCUUCUGG | CCAGAAGAUAAGCAACACC | Chmp | [161-179] | [132-150] |
| 164 | GAAUCUACGUCCUUGGAGG | CCUCCAAGGACGUAGAUUC | Chmp | [1879-1897] | [1850-1868] |
| 165 | CAAAAGAAGUCCAAAGCGG | CCGCUUUGGACUUCUUUUG | Chmp | [2210-2228] | [2181-2199] |
| 166 | UUGUUUCUUGGGCAAAAAU | AUUUUUGCCCAAGAAACAA | Chmp | [2068-2086] | [2039-2057] |
| 167 | UACGACCCAGAUACAGACA | UGUCUGUAUCUGGGUCGUA | Chmp | [1935-1953] | [1906-1924] |
| 168 | AGUGUUACGACCCAGAUAC | GUAUCUGGGUCGUAACACU | Chmp | [1930-1948] | [1901-1919] |
| 169 | GCGAAUGAUCACAGCAAUG | CAUUGCUGUGAUCAUUCGC | Chmp | [1676-1694] | [1647-1665] |
| 170 | ACGAGUGGCGAAUGAUCAC | GUGAUCAUUCGCCACUCGU | Chmp | [1669-1687] | [1640-1658] |
| 171 | AGUGGCGAAUGAUCACAGC | GCUGUGAUCAUUCGCCACU | Chmp | [1672-1690] | [1643-1661] |
| 172 | CUUAAUUCAGCUGAGUGUU | AACACUCAGCUGAAUUAAG | Chmp | [1635-1653] | [1606-1624] |
| 173 | AUGGAGCGCCUCAUUGAAU | AUUCAAUGAGGCGCUCCAU | | [582-600] | [553-571] |
| 174 | GCUAUGAUGGUCACACGUU | AACGUGUGACCAUCAUAGC | Chmp | [1897-1915] | [1868-1886] |
| 175 | AAAUAAAGAACAGACUAAC | GUUAGUCUGUUCUUUAUUU | Chmp | [2557-2575] | [2528-2546] |
| 176 | UAAAAUGACAUCUCAAAAG | CUUUUGAGAUGUCAUUUUA | Chmp | [2197-2215] | [2168-2186] |
| 177 | GUAAUUUUCCAAGGAAAAU | AUUUUCCUUGGAAAAUUAC | Chmp | [2542-2560] | [2513-2531] |
| 178 | GUUGCUUAUCUUCUGGAAC | GUUCCAGAAGAUAAGCAAC | Chmp | [164-182] | [135-153] |
| 179 | GUGGUGGUGUUGCUUAUCU | AGAUAAGCAACACCACCAC | | [156-174] | [127-145] |
| 180 | AACAGUGUGGAGAGGUAUG | CAUACCUCUCCACACUGUU | Chmp | [1497-1515] | [1468-1486] |
| 181 | AGUGUGGAGUGUUACGACC | GGUCGUAACACUCCACACU | Chmp | [1923-1941] | [1894-1912] |
| 182 | GGUGGUGGUGUUGCUUAUC | GAUAAGCAACACCACCACC | | [155-173] | [126-144] |
| 183 | AUGGUCAGGACCAGCUGAA | UUCAGCUGGUCCUGACCAU | Chmp | [1762-1780] | [1733-1751] |
| 184 | CAAGGCCAUGUUCACCAAC | GUUGGUGAACAUGGCCUUG | GP, Chn, Chmp | [506-524] | [477-495] |
| 185 | UCUACGUCCUUGGAGGCUA | UAGCCUCCAAGGACGUAGA | Chmp | [1882-1900] | [1853-1871] |
| 186 | AUGACAUCUCAAAAGAAGU | ACUUCUUUUGAGAUGUCAU | Chmp | [2201-2219] | [2172-2190] |
| 187 | AAAAGAAAAGACAGCACUG | CAGUGCUGUCUUUUCUUUU | Chmp | [2133-2151] | [2104-2122] |
| 188 | ACUUCCUGCAGAUGCAGCU | AGCUGCAUCUGCAGGAAGU | GP, Chn, Chmp | [1021-1039] | [992-1010] |
| 189 | GAGACGUGGACUUUCGUAG | CUACGAAAGUCCACGUCUC | Chmp | [1809-1827] | [1780-1798] |
| 190 | ACUCGUUGACGCCGAACUU | AAGUUCGGCGUCAACGAGU | Chmp | [1006-1024] | [977-995] |
| 191 | UCUUGGGCAAAAAUACAGU | ACUGUAUUUUUGCCCAAGA | | [2073-2091] | [2044-2062] |
| 192 | UGUUACGACCCAGAUACAG | CUGUAUCUGGGUCGUAACA | Chmp | [1932-1950] | [1903-1921] |
| 193 | CAUGAACGGUGCUGUCAUG | CAUGACAGCACCGUUCAUG | | [650-668] | [621-639] |
| 194 | CCAAUGCUGACACGAAGGA | UCCUUCGUGUCAGCAUUGG | Chmp | [1548-1566] | [1519-1537] |
| 195 | AGUUAUUUUGUUGAUAAGU | ACUUAUCAACAAAAUAACU | Chmp | [2517-2535] | [2488-2506] |
| 196 | UCAUCCAGCCCUGUCUUCA | UGAAGACAGGGCUGGAUGA | Chmp | [489-507] | [460-478] |
| 197 | AUGGUCACACGUUCCUGGA | UCCAGGAACGUGUGACCAU | Chmp | [1903-1921] | [1874-1892] |
| 198 | GGACAAACCGCCUUAAUUC | GAAUUAAGGCGGUUUGUCC | Chmp | [1624-1642] | [1595-1613] |
| 199 | GAUCAUACCAAGCAGGCCU | AGGCCUGCUUGGUAUGAUC | Chmp | [357-375] | [328-346] |
| 200 | UACCCAGAGAGGAACGAGU | ACUCGUUCCUCUCUGGGUA | Chmp | [1656-1674] | [1627-1645] |
| 201 | GUGGUGUUGCUUAUCUUCU | AGAAGAUAAGCAACACCAC | Chmp | [159-177] | [130-148] |
| 202 | AUGCCUCAGUGUUAAAAUG | CAUUUUAACACUGAGGCAU | Chmp | [2185-2203] | [2156-2174] |
| 203 | CAUCUACAUGCAUUUUGGG | CCCAAAAUGCAUGUAGAUG | Chmp | [803-821] | [774-792] |
| 204 | ACUAAAAGAAAAGACAGCA | UGCUGUCUUUUCUUUUAGU | Chmp | [2130-2148] | [2101-2119] |
| 205 | UGUGUGACGUCACACUGCA | UGCAGUGUGACGUCACACA | Chmp | [412-430] | [383-401] |
| 206 | UGUACCAGAUCGACAGCGU | ACGCUGUCGAUCUGGUACA | Chn, Chmp | [667-685] | [638-656] |
| 207 | ACGUCCUUGGAGGCUAUGA | UCAUAGCCUCCAAGGACGU | Chmp | [1885-1903] | [1856-1874] |
| 208 | AAACAGAGACGUGGACUUU | AAAGUCCACGUCUCUGUUU | Chmp | [1804-1822] | [1775-1793] |
| 209 | CCAAAGCGGGAAUCAUGUG | CACAUGAUUCCCGCUUUGG | Chmp | [2220-2238] | [2191-2209] |
| 210 | AACAGCGACGGUUCUACGU | ACGUAGAACCGUCGCUGUU | Chmp | [958-976] | [929-947] |
| 211 | GUGAUUUAUUCUUGGAUAC | GUAUCCAAGAAUAAAUCAC | Chmp | [2387-2405] | [2358-2376] |
| 212 | AUGUGAUUUAUUCUUGGAU | AUCCAAGAAUAAAUCACAU | | [2385-2403] | [2356-2374] |
| 213 | CCUGGUCAAGAUCUUCGAG | CUCGAAGAUCUUGACCAGG | Chmp | [1085-1103] | [1056-1074] |
| 214 | AAGUAACCCUGUAAUUUUC | GAAAAUUACAGGGUUACUU | Chmp | [2532-2550] | [2503-2521] |
| 215 | UCUUCCACGCCUGCAUCAA | UUGAUGCAGGCGUGGAAGA | Chmp | [919-937] | [890-908] |
| 216 | GUCCAUUGAGGGUAUCCAC | GUGGAUACCCUCAAUGGAC | Chmp | [554-572] | [525-543] |
| 217 | GUCCUUGGAGGCUAUGAUG | CAUCAUAGCCUCCAAGGAC | Chmp | [1887-1905] | [1858-1876] |
| 218 | GCUAUGAUGGUCAGGACCA | UGGUCCUGACCAUCAUAGC | Chmp | [1756-1774] | [1727-1745] |
| 219 | CACCACAACAGUGUGGAGA | UCUCCACACUGUUGUGGUG | Chmp | [1491-1509] | [1462-1480] |
| 220 | CAAGUACGACUGCGAACAG | CUGUUCGCAGUCGUACUUG | Chmp | [944-962] | [915-933] |
| 221 | UUUCCAAGGAAAAUAAAGA | UCUUUAUUUUCCUUGGAAA | Chmp | [2547-2565] | [2518-2536] |
| 222 | UGGAAAGAGCAGGCUUCCA | UGGAAGCCUGCUCUUUCCA | Chmp | [2293-2311] | [2264-2282] |
| 223 | AAAGACAGCACUGCAAAUA | UAUUUGCAGUGCUGUCUUU | Chmp | [2139-2157] | [2110-2128] |
| 224 | GAUGUGGAAACAGAGACGU | ACGUCUCUGUUUCCACAUC | Chmp | [1797-1815] | [1768-1786] |
| 225 | UAAAAGAAAAGACAGCACU | AGUGCUGUCUUUUCUUUUA | Chmp | [2132-2150] | [2103-2121] |
| 226 | AUGGGCCUGUACAUAGAAG | CUUCUAUGUACAGGCCCAU | Chmp | [2467-2485] | [2438-2456] |
| 227 | CUGUAUCUAUGCUGCUGGG | CCCAGCAGCAUAGAUACAG | Chmp | [1736-1754] | [1707-1725] |
| 228 | AGCAGAUUGGCUGUGUGGA | UCCACACAGCCAAUCUGCU | | [760-778] | [731-749] |
| 229 | CAACUGUAUCUAUGCUGCU | AGCAGCAUAGAUACAGUUG | Chmp | [1733-1751] | [1704-1722] |
| 230 | AGUGUUACUACCCAGAGAG | CUCUCUGGGUAGUAACACU | Chmp | [1648-1666] | [1619-1637] |
| 231 | UAAUUUUCCAAGGAAAAUA | UAUUUUCCUUGGAAAAUUA | Chmp | [2543-2561] | [2514-2532] |
| 232 | GGCACUUUUGUUUCUUGGG | CCCAAGAAACAAAAGUGCC | Chmp | [2061-2079] | [2032-2050] |
| 233 | UGUGGAAAGAGCAGGCUUC | GAAGCCUGCUCUUUCCACA | Chmp | [2291-2309] | [2262-2280] |
| 234 | UGCAGAAGUGCGAGAUCCU | AGGAUCUCGCACUUCUGCA | GP, Chn, Chmp | [1039-1057] | [1010-1028] |
| 235 | ACAUAGAAGCCACCGGAUG | CAUCCGGUGGCUUCUAUGU | Chmp | [2477-2495] | [2448-2466] |
| 236 | GCACAACUGUAUCUAUGCU | AGCAUAGAUACAGUUGUGC | Chmp | [1730-1748] | [1701-1719] |
| 237 | AACUGGUGACCCUCAUCAG | CUGAUGAGGGUCACCAGUU | Chmp | [865-883] | [836-854] |
| 238 | AGGCACUUUUGUUUCUUGG | CCAAGAAACAAAAGUGCCU | Chmp | [2060-2078] | [2031-2049] |
| 239 | ACCUGUUGAGGCACUUUUG | CAAAAGUGCCUCAACAGGU | Chmp | [2052-2070] | [2023-2041] |
| 240 | UGAUCACAGCAAUGAACAC | GUGUUCAUUGCUGUGAUCA | Chmp | [1681-1699] | [1652-1670] |
| 241 | CAUCCACCACAACAGUGUG | CACACUGUUGUGGUGGAUG | Chmp | [1487-1505] | [1458-1476] |
| 242 | CUGCAUCCACCACAACAGU | ACUGUUGUGGUGGAUGCAG | Chmp | [1484-1502] | [1455-1473] |
| 243 | CUUGGAGGCUAUGAUGGUC | GACCAUCAUAGCCUCCAAG | Chmp | [1890-1908] | [1861-1879] |
| 244 | CGUCCUGCACAACUGUAUC | GAUACAGUUGUGCAGGACG | Chmp | [1724-1742] | [1695-1713] |
| 245 | GAGGAUCAUACCAAGCAGG | CCUGCUUGGUAUGAUCCUC | Chmp | [354-372] | [325-343] |
| 246 | AUGAUCACAGCAAUGAACA | UGUUCAUUGCUGUGAUCAU | Chmp | [1680-1698] | [1651-1669] |
| 247 | AAUGCUGACACGAAGGAUC | GAUCCUUCGUGUCAGCAUU | Chmp | [1550-1568] | [1521-1539] |
| 248 | GCAUCCACCACAACAGUGU | ACACUGUUGUGGUGGAUGC | Chmp | [1486-1504] | [1457-1475] |
| 249 | GUCACACUGCAGGUCAAGU | ACUUGACCUGCAGUGUGAC | Chmp | [420-438] | [391-409] |
| 250 | UGUGGAAACAGAGACGUGG | CCACGUCUCUGUUUCCACA | Chmp | [1799-1817] | [1770-1788] |
| 251 | ACCAGAUCGACAGCGUUGU | ACAACGCUGUCGAUCUGGU | Chmp | [670-688] | [641-659] |
| 252 | ACUAGUGUCUUUCACCCUG | CAGGGUGAAAGACACUAGU | Chmp | [2574-2592] | [2545-2563] |
| 253 | AUUUAUUCUUGGAUACCUG | CAGGUAUCCAAGAAUAAAU | Chmp | [2390-2408] | [2361-2379] |
| 254 | UCUUCAACCUGUCCCACUG | CAGUGGGACAGGUUGAAGA | GP, Chmp | [844-862] | [815-833] |
| 255 | UGACCAGCAGAACUGUACC | GGUACAGUUCUGCUGGUCA | Chmp | [2036-2054] | [2007-2025] |
| 256 | GGACUACCUGGUCAAGAUC | GAUCUUGACCAGGUAGUCC | Chmp | [1079-1097] | [1050-1068] |
| 257 | CUGCAUCAACUGGGUCAAG | CUUGACCCAGUUGAUGCAG | GP, Chmp | [929-947] | [900-918] |
| 258 | CAAUCGUCUCCUUUAUGCC | GGCAUAAAGGAGACGAUUG | | [1589-1607] | [1560-1578] |
| 259 | UUGGAGGCUAUGAUGGUCA | UGACCAUCAUAGCCUCCAA | Chmp | [1891-1909] | [1862-1880] |
| 260 | UCACACUGCAGGUCAAGUA | UACUUGACCUGCAGUGUGA | Chmp | [421-439] | [392-410] |
| 261 | UACACCCUGGAGGAUCAUA | UAUGAUCCUCCAGGGUGUA | Chmp | [345-363] | [316-334] |
| 262 | AGAAUCUACGUCCUUGGAG | CUCCAAGGACGUAGAUUCU | Chmp | [1878-1896] | [1849-1867] |
| 263 | AGAUCUUCGAGGAGCUCAC | GUGAGCUCCUCGAAGAUCU | Chmp | [1093-1111] | [1064-1082] |
| 264 | AAGCAGGCCUUUGGCAUCA | UGAUGCCAAAGGCCUGCUU | Chmp | [366-384] | [337-355] |
| 265 | UGAUGGUCACACGUUCCUG | CAGGAACGUGUGACCAUCA | Chmp | [1901-1919] | [1872-1890] |
| 266 | CAGCAAUGAACACCAUCCG | CGGAUGGUGUUCAUUGCUG | Chmp | [1687-1705] | [1658-1676] |
| 267 | GACUAAAAGAAAAGACAGC | GCUGUCUUUUCUUUUAGUC | Chmp | [2129-2147] | [2100-2118] |
| 268 | CUGCAAAUAACCCAUCUUC | GAAGAUGGGUUAUUUGCAG | Chmp | [2149-2167] | [2120-2138] |
| 269 | GUCAUGAACGGUGCUGUCA | UGACAGCACCGUUCAUGAC | | [648-666] | [619-637] |
| 270 | AUAAGUAACCCUGUAAUUU | AAAUUACAGGGUUACUUAU | Chmp | [2530-2548] | [2501-2519] |
| 271 | AAACCGCCUUAAUUCAGCU | AGCUGAAUUAAGGCGGUUU | Chmp | [1628-1646] | [1599-1617] |
| 272 | AGUGUGGAGAGGUAUGAGC | GCUCAUACCUCUCCACACU | Chmp | [1500-1518] | [1471-1489] |
| 273 | GAACUUCCUGCAGAUGCAG | CUGCAUCUGCAGGAAGUUC | Chmp | [1019-1037] | [990-1008] |
| 274 | ACUACCCAGAGAGGAACGA | UCGUUCCUCUCUGGGUAGU | Chmp | [1654-1672] | [1625-1643] |
| 275 | UGCGUCCUGCACAACUGUA | UACAGUUGUGCAGGACGCA | Chmp | [1722-1740] | [1693-1711] |
| 276 | UGUUACUACCCAGAGAGGA | UCCUCUCUGGGUAGUAACA | Chmp | [1650-1668] | [1621-1639] |
| 277 | CUACCUGGUCAAGAUCUUC | GAAGAUCUUGACCAGGUAG | Chmp | [1082-1100] | [1053-1071] |
| 278 | ACGAUGUGGAAACAGAGAC | GUCUCUGUUUCCACAUCGU | Chmp | [1795-1813] | [1766-1784] |
| 279 | CAUGUACCAGAUCGACAGC | GCUGUCGAUCUGGUACAUG | Chn, Chmp | [665-683] | [636-654] |
| 280 | GUCAAGUACGACUGCGAAC | GUUCGCAGUCGUACUUGAC | Chmp | [942-960] | [913-931] |
| 281 | ACAAACCGCCUUAAUUCAG | CUGAAUUAAGGCGGUUUGU | Chmp | [1626-1644] | [1597-1615] |
| 282 | AACUAGUGUCUUUCACCCU | AGGGUGAAAGACACUAGUU | Chmp | [2573-2591] | [2544-2562] |
| 283 | GAUUGGCUGUGUGGAGUUG | CAACUCCACACAGCCAAUC | | [764-782] | [735-753] |
| 284 | AUUCUUGGAUACCUGGGAG | CUCCCAGGUAUCCAAGAAU | Chmp | [2394-2412] | [2365-2383] |
| 285 | UGUCCUCCACGUCAUGAAC | GUUCAUGACGUGGAGGACA | Chmp | [638-656] | [609-627] |
| 286 | UCUCAAAAGAAGUCCAAAG | CUUUGGACUUCUUUUGAGA | Chmp | [2207-2225] | [2178-2196] |
| 287 | AUGUGGAAACAGAGACGUG | CACGUCUCUGUUUCCACAU | Chmp | [1798-1816] | [1769-1787] |
| 288 | AAGAAGUCCAAAGCGGGAA | UUCCCGCUUUGGACUUCUU | Chmp | [2213-2231] | [2184-2202] |
| 289 | AUCAUUGUUUUUGUACAAA | UUUGUACAAAAACAAUGAU | Chmp | [2104-2122] | [2075-2093] |
| 290 | UUGGCAUCAUGAACGAGCU | AGCUCGUUCAUGAUGCCAA | Rat, Chmp | [376-394] | [347-365] |
| 291 | CAAACCGCCUUAAUUCAGC | GCUGAAUUAAGGCGGUUUG | Chmp | [1627-1645] | [1598-1616] |
| 292 | CUCAAUCGUCUCCUUUAUG | CAUAAAGGAGACGAUUGAG | | [1587-1605] | [1558-1576] |
| 293 | AAGUCCAAAGCGGGAAUCA | UGAUUCCCGCUUUGGACUU | Chmp | [2216-2234] | [2187-2205] |
| 294 | AGCUACCUGGAGGCUUACA | UGUAAGCCUCCAGGUAGCU | Chmp | [1203-1221] | [1174-1192] |
| 295 | AUAACCCAUCUUCCGGGAA | UUCCCGGAAGAUGGGUUAU | Chmp | [2155-2173] | [2126-2144] |
| 296 | CUCAAAAGAAGUCCAAAGC | GCUUUGGACUUCUUUUGAG | Chmp | [2208-2226] | [2179-2197] |
| 297 | GACAGCACUGCAAAUAACC | GGUUAUUUGCAGUGCUGUC | Chmp | [2142-2160] | [2113-2131] |
| 298 | AGCUGUCACCAUGUGAUUU | AAAUCACAUGGUGACAGCU | | [2375-2393] | [2346-2364] |
| 299 | AAGGACUACCUGGUCAAGA | UCUUGACCAGGUAGUCCUU | Chmp | [1077-1095] | [1048-1066] |
| 300 | GACCCAGAUACAGACACCU | AGGUGUCUGUAUCUGGGUC | Chmp | [1938-1956] | [1909-1927] |
| 301 | CAAGCAAGAGGAGUUCUUC | GAAGAACUCCUCUUGCUUG | Chmp | [830-848] | [801-819] |
| 302 | CAAGAGGAGUUCUUCAACC | GGUUGAAGAACUCCUCUUG | Chmp | [834-852] | [805-823] |
| 303 | AUCACAGCAAUGAACACCA | UGGUGUUCAUUGCUGUGAU | Chmp | [1683-1701] | [1654-1672] |
| 304 | UCACCAUGUGAUUUAUUCU | AGAAUAAAUCACAUGGUGA | | [2380-2398] | [2351-2369] |
| 305 | AGCAGAACUGUACCUGUUG | CAACAGGUACAGUUCUGCU | Chmp | [2041-2059] | [2012-2030] |
| 306 | AAAAAUACAGUCCAAUGGG | CCCAUUGGACUGUAUUUUU | | [2081-2099] | [2052-2070] |
| 307 | AAUGAUCACAGCAAUGAAC | GUUCAUUGCUGUGAUCAUU | Chmp | [1679-1697] | [1650-1668] |
| 308 | UAUUUUGUUGAUAAGUAAC | GUUACUUAUCAACAAAAUA | Chmp | [2520-2538] | [2491-2509] |
| 309 | UCCAAAGCGGGAAUCAUGU | ACAUGAUUCCCGCUUUGGA | Chmp | [2219-2237] | [2190-2208] |
| 310 | ACCCUGGAGGAUCAUACCA | UGGUAUGAUCCUCCAGGGU | Chmp | [348-366] | [319-337] |
| 311 | AGACGUGGACUUUCGUAGC | GCUACGAAAGUCCACGUCU | Chmp | [1810-1828] | [1781-1799] |
| 312 | GUCCAAAGCGGGAAUCAUG | CAUGAUUCCCGCUUUGGAC | Chmp | [2218-2236] | [2189-2207] |
| 313 | UGCAAAUAACCCAUCUUCC | GGAAGAUGGGUUAUUUGCA | Chmp | [2150-2168] | [2121-2139] |
| 314 | UAAUUCAGCUGAGUGUUAC | GUAACACUCAGCUGAAUUA | Chmp | [1637-1655] | [1608-1626] |
| 315 | AAGAGGAGUUCUUCAACCU | AGGUUGAAGAACUCCUCUU | Chmp | [835-853] | [806-824] |
| 316 | UCUGGAAAUGUGGUUCCCA | UGGGAACCACAUUUCCAGA | Chmp | [2445-2463] | [2416-2434] |
| 317 | GCAAAUAACCCAUCUUCCG | CGGAAGAUGGGUUAUUUGC | Chmp | [2151-2169] | [2122-2140] |
| 318 | UUGAGGCACUUUUGUUUCU | AGAAACAAAAGUGCCUCAA | Chmp | [2057-2075] | [2028-2046] |
| 319 | UCACACGUUCCUGGACAGU | ACUGUCCAGGAACGUGUGA | GP, Chn, Chmp | [1907-1925] | [1878-1896] |
| 320 | CUUUGGCAUCAUGAACGAG | CUCGUUCAUGAUGCCAAAG | Chmp | [374-392] | [345-363] |
| 321 | AUCAUACCAAGCAGGCCUU | AAGGCCUGCUUGGUAUGAU | Chmp | [358-376] | [329-347] |
| 322 | AGGUGACCCGAAUGACAUC | GAUGUCAUUCGGGUCACCU | Chmp | [1963-1981] | [1934-1952] |
| 323 | AAUAACCCAUCUUCCGGGA | UCCCGGAAGAUGGGUUAUU | Chmp | [2154-2172] | [2125-2143] |
| 324 | ACCACAACAGUGUGGAGAG | CUCUCCACACUGUUGUGGU | Chmp | [1492-1510] | [1463-1481] |
| 325 | AAGAACAGACUAACUAGUG | CACUAGUUAGUCUGUUCUU | Chmp | [2562-2580] | [2533-2551] |
| 326 | GUUUUUGUACAAAAACCGG | CCGGUUUUUGUACAAAAAC | Chmp | [2110-2128] | [2081-2099] |
| 327 | UCAAAAGAAGUCCAAAGCG | CGCUUUGGACUUCUUUUGA | Chmp | [2209-2227] | [2180-2198] |
| 328 | UGUGUCCUCCACGUCAUGA | UCAUGACGUGGAGGACACA | Chmp | [636-654] | [607-625] |
| 329 | UCAGCUGAGUGUUACUACC | GGUAGUAACACUCAGCUGA | Chmp | [1641-1659] | [1612-1630] |
| 330 | UGUCUUCAAGGCCAUGUUC | GAACAUGGCCUUGAAGACA | Chmp | [500-518] | [471-489] |
| 331 | CACACUGCAGGUCAAGUAC | GUACUUGACCUGCAGUGUG | Chmp | [422-440] | [393-411] |
| 332 | UGGAAAUGUGGUUCCCAGG | CCUGGGAACCACAUUUCCA | Chmp | [2447-2465] | [2418-2436] |
| 333 | ACGACCCAGAUACAGACAC | GUGUCUGUAUCUGGGUCGU | Chmp | [1936-1954] | [1907-1925] |
| 334 | AUCGAUGGCCACAUCUAUG | CAUAGAUGUGGCCAUCGAU | Chmp | [1446-1464] | [1417-1435] |
| 335 | GUGUUACUACCCAGAGAGG | CCUCUCUGGGUAGUAACAC | Chmp | [1649-1667] | [1620-1638] |
| 336 | GACUACCUGGUCAAGAUCU | AGAUCUUGACCAGGUAGUC | Chmp | [1080-1098] | [1051-1069] |
| 337 | ACUGCAGGUCAAGUACCAG | CUGGUACUUGACCUGCAGU | Chmp | [425-443] | [396-414] |
| 338 | AAAAGAAGUCCAAAGCGGG | CCCGCUUUGGACUUCUUUU | Chmp | [2211-2229] | [2182-2200] |
| 339 | UGCUGUCAUGUACCAGAUC | GAUCUGGUACAUGACAGCA | | [659-677] | [630-648] |
| 340 | AAGGCCAUGUUCACCAACG | CGUUGGUGAACAUGGCCUU | GP, Chn, Chmp | [507-525] | [478-496] |
| 341 | ACCAUGUGAUUUAUUCUUG | CAAGAAUAAAUCACAUGGU | | [2382-2400] | [2353-2371] |
| 342 | AUACAGACACCUGGAGCGA | UCGCUCCAGGUGUCUGUAU | Chmp | [1945-1963] | [1916-1934] |
| 343 | AUGAUGGUCACACGUUCCU | AGGAACGUGUGACCAUCAU | Chmp | [1900-1918] | [1871-1889] |
| 344 | UAACCCAUCUUCCGGGAAG | CUUCCCGGAAGAUGGGUUA | Chmp | [2156-2174] | [2127-2145] |
| 345 | ACAGAGACGUGGACUUUCG | CGAAAGUCCACGUCUCUGU | Chmp | [1806-1824] | [1777-1795] |
| 346 | CCUGUACAUAGAAGCCACC | GGUGGCUUCUAUGUACAGG | Chmp | [2472-2490] | [2443-2461] |
| 347 | AAGUGCGAGAUCCUGCAGU | ACUGCAGGAUCUCGCACUU | Chmp | [1044-1062] | [1015-1033] |
| 348 | UGAGUGUUACUACCCAGAG | CUCUGGGUAGUAACACUCA | Chmp | [1646-1664] | [1617-1635] |
| 349 | AGAGGAGUUCUUCAACCUG | CAGGUUGAAGAACUCCUCU | Chmp | [836-854] | [807-825] |
| 350 | AACUGGGUCAAGUACGACU | AGUCGUACUUGACCCAGUU | GP, Chn, Chmp | [936-954] | [907-925] |
| 351 | GUUGAGGCACUUUUGUUUC | GAAACAAAAGUGCCUCAAC | Chmp | [2056-2074] | [2027-2045] |
| 352 | AUGGCCACAUCUAUGCCGU | ACGGCAUAGAUGUGGCCAU | Chmp | [1450-1468] | [1421-1439] |
| 353 | ACUUCGCUGAGCAGAUUGG | CCAAUCUGCUCAGCGAAGU | Chmp | [751-769] | [722-740] |
| 354 | CUAACUAGUGUCUUUCACC | GGUGAAAGACACUAGUUAG | Chmp | [2571-2589] | [2542-2560] |
| 355 | CUGCACAACUGUAUCUAUG | CAUAGAUACAGUUGUGCAG | Ms, Chmp | [1728-1746] | [1699-1717] |
| 356 | ACGUCAUGAACGGUGCUGU | ACAGCACCGUUCAUGACGU | | [646-664] | [617-635] |
| 357 | AGAUACAGACACCUGGAGC | GCUCCAGGUGUCUGUAUCU | Chmp | [1943-1961] | [1914-1932] |
| 358 | ACUGGGUCAAGUACGACUG | CAGUCGUACUUGACCCAGU | GP, Chn, Chmp | [937-955] | [908-926] |
| 359 | ACUAACUAGUGUCUUUCAC | GUGAAAGACACUAGUUAGU | Chmp | [2570-2588] | [2541-2559] |
| 360 | AACAGAGACGUGGACUUUC | GAAAGUCCACGUCUCUGUU | Chmp | [1805-1823] | [1776-1794] |
| 361 | CUUGGGCAAAAAUACAGUC | GACUGUAUUUUUGCCCAAG | | [2074-2092] | [2045-2063] |
| 362 | GUUCUUCAACCUGUCCCAC | GUGGGACAGGUUGAAGAAC | GP, Chmp | [842-860] | [813-831] |
| 363 | AGGUCAAGUACCAGGAUGC | GCAUCCUGGUACUUGACCU | Chmp | [430-448] | [401-419] |
| 364 | UCAAGUACCAGGAUGCACC | GGUGCAUCCUGGUACUUGA | Chmp | [433-451] | [404-422] |
| 365 | AGCGCCUCAUUGAAUUCGC | GCGAAUUCAAUGAGGCGCU | | [586-604] | [557-575] |
| 366 | CAACUGGGUCAAGUACGAC | GUCGUACUUGACCCAGUUG | GP, Chn, Chmp | [935-953] | [906-924] |
| 367 | UGUCAUGUACCAGAUCGAC | GUCGAUCUGGUACAUGACA | Chmp | [662-680] | [633-651] |
| 368 | UGCUUAUCUUCUGGAACCC | GGGUUCCAGAAGAUAAGCA | Chmp | [166-184] | [137-155] |
| 369 | UUGCUUAUCUUCUGGAACC | GGUUCCAGAAGAUAAGCAA | Chmp | [165-183] | [136-154] |
| 370 | CAUACCAAGCAGGCCUUUG | CAAAGGCCUGCUUGGUAUG | Chmp | [360-378] | [331-349] |
| 371 | GAACUGUACCUGUUGAGGC | GCCUCAACAGGUACAGUUC | Chmp | [2045-2063] | [2016-2034] |
| 372 | AGUAUCAUUGUUUUUGUAC | GUACAAAAACAAUGAUACU | Chmp | [2101-2119] | [2072-2090] |
| 373 | UUGACCAGCAGAACUGUAC | GUACAGUUCUGCUGGUCAA | Chmp | [2035-2053] | [2006-2024] |
| 374 | UGGCAUCAUGAACGAGCUG | CAGCUCGUUCAUGAUGCCA | Chmp | [377-395] | [348-366] |
| 375 | CACAGCAAUGAACACCAUC | GAUGGUGUUCAUUGCUGUG | Chmp | [1685-1703] | [1656-1674] |
| 376 | AUUGAAUUCGCCUACACGG | CCGUGUAGGCGAAUUCAAU | | [594-612] | [565-583] |
| 377 | UUUGUUGAUAAGUAACCCU | AGGGUUACUUAUCAACAAA | Chmp | [2523-2541] | [2494-2512] |
| 378 | AUCCAGCCCUGUCUUCAAG | CUUGAAGACAGGGCUGGAU | Chmp | [491-509] | [462-480] |
| 379 | GGAGUUCUUCAACCUGUCC | GGACAGGUUGAAGAACUCC | GP, Chmp | [839-857] | [810-828] |
| 380 | UUCUUGGGCAAAAAUACAG | CUGUAUUUUUGCCCAAGAA | | [2072-2090] | [2043-2061] |
| 381 | UUCAAGGCCAUGUUCACCA | UGGUGAACAUGGCCUUGAA | GP, Chn, Chmp | [504-522] | [475-493] |
| 382 | CAAAGCGGGAAUCAUGUGC | GCACAUGAUUCCCGCUUUG | Chmp | [2221-2239] | [2192-2210] |
| 383 | UUUUCCAAGGAAAAUAAAG | CUUUAUUUUCCUUGGAAAA | Chmp | [2546-2564] | [2517-2535] |
| 384 | CUGGUCAAGAUCUUCGAGG | CCUCGAAGAUCUUGACCAG | Chmp | [1086-1104] | [1057-1075] |
| 385 | UUCCACGCCUGCAUCAACU | AGUUGAUGCAGGCGUGGAA | GP, Chmp | [921-939] | [892-910] |
| 386 | ACAACUGUAUCUAUGCUGC | GCAGCAUAGAUACAGUUGU | Chmp | [1732-1750] | [1703-1721] |
| 387 | AACAGACUAACUAGUGUCU | AGACACUAGUUAGUCUGUU | Chmp | [2565-2583] | [2536-2554] |
| 388 | AUACCAAGCAGGCCUUUGG | CCAAAGGCCUGCUUGGUAU | Chmp | [361-379] | [332-350] |
| 389 | UUCUUCAACCUGUCCCACU | AGUGGGACAGGUUGAAGAA | GP, Chmp | [843-861] | [814-832] |
| 390 | AACUGUAUCUAUGCUGCUG | CAGCAGCAUAGAUACAGUU | Chmp | [1734-1752] | [1705-1723] |
| 391 | UUGUACAAAAACCGGGACU | AGUCCCGGUUUUUGUACAA | Chmp | [2114-2132] | [2085-2103] |
| 392 | AACCCUGUAAUUUUCCAAG | CUUGGAAAAUUACAGGGUU | Chmp | [2536-2554] | [2507-2525] |
| 393 | UGGGCCUGUACAUAGAAGC | GCUUCUAUGUACAGGCCCA | Chmp | [2468-2486] | [2439-2457] |
| 394 | UUUAUUCUUGGAUACCUGG | CCAGGUAUCCAAGAAUAAA | Chmp | [2391-2409] | [2362-2380] |
| 395 | AUCGUCUCCUUUAUGCCGU | ACGGCAUAAAGGAGACGAU | | [1591-1609] | [1562-1580] |
| 396 | AAUGUGGUUCCCAGGGAUG | CAUCCCUGGGAACCACAUU | Chmp | [2451-2469] | [2422-2440] |
| 397 | UAACUAGUGUCUUUCACCC | GGGUGAAAGACACUAGUUA | Chmp | [2572-2590] | [2543-2561] |
| 398 | ACAGCACUGCAAAUAACCC | GGGUUAUUUGCAGUGCUGU | Chmp | [2143-2161] | [2114-2132] |
| 399 | CACAACAGUGUGGAGAGGU | ACCUCUCCACACUGUUGUG | Chmp | [1494-1512] | [1465-1483] |
| 400 | AUGUACGCCUCCACUGAGU | ACUCAGUGGAGGCGUACAU | Chmp | [279-297] | [250-268] |
| 401 | UACUUCCGACAGUCGCUCA | UGAGCGACUGUCGGAAGUA | Rat, Ms, Chmp | [1185-1203] | [1156-1174] |
| 402 | GCUGUCAUGUACCAGAUCG | CGAUCUGGUACAUGACAGC | Chmp | [660-678] | [631-649] |
| 403 | UGUUUUUGUACAAAAACCG | CGGUUUUUGUACAAAAACA | Chmp | [2109-2127] | [2080-2098] |
| 404 | UGUACAUAGAAGCCACCGG | CCGGUGGCUUCUAUGUACA | Chmp | [2474-2492] | [2445-2463] |
| 405 | CAAAUAACCCAUCUUCCGG | CCGGAAGAUGGGUUAUUUG | Chmp | [2152-2170] | [2123-2141] |
| 406 | AGAACUGUACCUGUUGAGG | CCUCAACAGGUACAGUUCU | Chmp | [2044-2062] | [2015-2033] |
| 407 | GUCAAGUACCAGGAUGCAC | GUGCAUCCUGGUACUUGAC | Chmp | [432-450] | [403-421] |
| 408 | GUCAUCGAUGGCCACAUCU | AGAUGUGGCCAUCGAUGAC | Chn, Chmp | [1443-1461] | [1414-1432] |
| 409 | UUUUUGUACAAAAACCGGG | CCCGGUUUUUGUACAAAAA | Chmp | [2111-2129] | [2082-2100] |
| 410 | UCAAUCGUCUCCUUUAUGC | GCAUAAAGGAGACGAUUGA | | [1588-1606] | [1559-1577] |
| 411 | CAAGAUCUUCGAGGAGCUC | GAGCUCCUCGAAGAUCUUG | Chmp | [1091-1109] | [1062-1080] |
| 412 | AAGAUCUUCGAGGAGCUCA | UGAGCUCCUCGAAGAUCUU | Chmp | [1092-1110] | [1063-1081] |
| 413 | AUGGACAGUUAUUUUGUUG | CAACAAAAUAACUGUCCAU | Chmp | [2511-2529] | [2482-2500] |
| 414 | UUACUACCCAGAGAGGAAC | GUUCCUCUCUGGGUAGUAA | Chmp | [1652-1670] | [1623-1641] |
| 415 | AUCAACUGGGUCAAGUACG | CGUACUUGACCCAGUUGAU | GP, Chn, Chmp | [933-951] | [904-922] |
| 416 | AACUUCGCUGAGCAGAUUG | CAAUCUGCUCAGCGAAGUU | Chmp | [750-768] | [721-739] |
| 417 | AUUCAGCUGAGUGUUACUA | UAGUAACACUCAGCUGAAU | Chmp | [1639-1657] | [1610-1628] |
| 418 | ACUGUAUCUAUGCUGCUGG | CCAGCAGCAUAGAUACAGU | Chmp | [1735-1753] | [1706-1724] |
| 419 | AAUCGUCUCCUUUAUGCCG | CGGCAUAAAGGAGACGAUU | | [1590-1608] | [1561-1579] |
| 420 | UUUUGUUGAUAAGUAACCC | GGGUUACUUAUCAACAAAA | Chmp | [2522-2540] | [2493-2511] |
| 421 | GAGCGCCUCAUUGAAUUCG | CGAAUUCAAUGAGGCGCUC | | [585-603] | [556-574] |
| 422 | AUUGUUUUUGUACAAAAAC | GUUUUUGUACAAAAACAAU | Chmp | [2107-2125] | [2078-2096] |
| 423 | AAAGAAAAGACAGCACUGC | GCAGUGCUGUCUUUUCUUU | Chmp | [2134-2152] | [2105-2123] |
| 424 | UUGUUGAUAAGUAACCCUG | CAGGGUUACUUAUCAACAA | Chmp | [2524-2542] | [2495-2513] |
| 425 | CUGUACAUAGAAGCCACCG | CGGUGGCUUCUAUGUACAG | Chmp | [2473-2491] | [2444-2462] |
| 426 | UUCGCUGAGCAGAUUGGCU | AGCCAAUCUGCUCAGCGAA | Chmp | [753-771] | [724-742] |
| 427 | ACCCAGAUACAGACACCUG | CAGGUGUCUGUAUCUGGGU | Chmp | [1939-1957] | [1910-1928] |
| 428 | CUGGAGGAUCAUACCAAGC | GCUUGGUAUGAUCCUCCAG | Chmp | [351-369] | [322-340] |
| 429 | UUUGUACAAAAACCGGGAC | GUCCCGGUUUUUGUACAAA | Chmp | [2113-2131] | [2084-2102] |
| 430 | AUGAACGGUGCUGUCAUGU | ACAUGACAGCACCGUUCAU | | [651-669] | [622-640] |
| 431 | UCCACCACAACAGUGUGGA | UCCACACUGUUGUGGUGGA | Chmp | [1489-1507] | [1460-1478] |
| 432 | AAGUACGACUGCGAACAGC | GCUGUUCGCAGUCGUACUU | Chmp | [945-963] | [916-934] |
| 433 | GUGUCCAUUGAGGGUAUCC | GGAUACCCUCAAUGGACAC | Chmp | [552-570] | [523-541] |
| 434 | UUAUUCUUGGAUACCUGGG | CCCAGGUAUCCAAGAAUAA | Chmp | [2392-2410] | [2363-2381] |
| 435 | AGUAACCCUGUAAUUUUCC | GGAAAAUUACAGGGUUACU | Chmp | [2533-2551] | [2504-2522] |
| 436 | AUGCUGACACGAAGGAUCG | CGAUCCUUCGUGUCAGCAU | Chmp | [1551-1569] | [1522-1540] |
| 437 | AUGGAGGUGGUGUCCAUUG | CAAUGGACACCACCUCCAU | Rat, Chmp | [543-561] | [514-532] |
| 438 | CAGCUGAGUGUUACUACCC | GGGUAGUAACACUCAGCUG | Chmp | [1642-1660] | [1613-1631] |
| 439 | UUGUUUUUGUACAAAAACC | GGUUUUUGUACAAAAACAA | Chmp | [2108-2126] | [2079-2097] |
| 440 | AGGAUCAUACCAAGCAGGC | GCCUGCUUGGUAUGAUCCU | Chmp | [355-373] | [326-344] |
| 441 | CAUGACCAAUCAGUGGUCG | CGACCACUGAUUGGUCAUG | Chn, Chmp | [1379-1397] | [1350-1368] |
| 442 | AACGGUGCUGUCAUGUACC | GGUACAUGACAGCACCGUU | | [654-672] | [625-643] |
| 443 | UACUACCCAGAGAGGAACG | CGUUCCUCUCUGGGUAGUA | Chmp | [1653-1671] | [1624-1642] |
| 444 | UUCAGCUGAGUGUUACUAC | GUAGUAACACUCAGCUGAA | Chmp | [1640-1658] | [1611-1629] |
| 445 | CUGUCCUCAAUCGUCUCCU | AGGAGACGAUUGAGGACAG | | [1582-1600] | [1553-1571] |
| 446 | AUGAUGGUCAGGACCAGCU | AGCUGGUCCUGACCAUCAU | Chmp | [1759-1777] | [1730-1748] |
| 447 | AAAGCGGGAAUCAUGUGCC | GGCACAUGAUUCCCGCUUU | Chmp | [2222-2240] | [2193-2211] |
| 448 | AUCUCCAUGGGCGAGAAGU | ACUUCUCGCCCAUGGAGAU | GP, Chn, Chmp | [618-636] | [589-607] |
| 449 | UAUGAUGGUCACACGUUCC | GGAACGUGUGACCAUCAUA | Chmp | [1899-1917] | [1870-1888] |
| 450 | UGCACAACUGUAUCUAUGC | GCAUAGAUACAGUUGUGCA | Ms, Chmp | [1729-1747] | [1700-1718] |
| 451 | UCGAUGGCCACAUCUAUGC | GCAUAGAUGUGGCCAUCGA | Chmp | [1447-1465] | [1418-1436] |
| 452 | UGAUUUAUUCUUGGAUACC | GGUAUCCAAGAAUAAAUCA | Chmp | [2388-2406] | [2359-2377] |
| 453 | GAUCACAGCAAUGAACACC | GGUGUUCAUUGCUGUGAUC | Chmp | [1682-1700] | [1653-1671] |
| 454 | UUCUUGGAUACCUGGGAGG | CCUCCCAGGUAUCCAAGAA | Chmp | [2395-2413] | [2366-2384] |
| 455 | AAAUAACCCAUCUUCCGGG | CCCGGAAGAUGGGUUAUUU | Chmp | [2153-2171] | [2124-2142] |
| 456 | AUGACCAAUCAGUGGUCGC | GCGACCACUGAUUGGUCAU | Chn, Chmp | [1380-1398] | [1351-1369] |
| 457 | UACCAGAUCGACAGCGUUG | CAACGCUGUCGAUCUGGUA | Chmp | [669-687] | [640-658] |
| 458 | UCCAUUGAGGGUAUCCACC | GGUGGAUACCCUCAAUGGA | Chmp | [555-573] | [526-544] |
| 459 | AACCGCCUUAAUUCAGCUG | CAGCUGAAUUAAGGCGGUU | Chmp | [1629-1647] | [1600-1618] |
| 460 | AUUUUGUUGAUAAGUAACC | GGUUACUUAUCAACAAAAU | Chmp | [2521-2539] | [2492-2510] |
| 461 | ACUGUACCUGUUGAGGCAC | GUGCCUCAACAGGUACAGU | Chmp | [2047-2065] | [2018-2036] |
| 462 | UUGAAUUCGCCUACACGGC | GCCGUGUAGGCGAAUUCAA | | [595-613] | [566-584] |
| 463 | AAUGAACACCAUCCGAAGC | GCUUCGGAUGGUGUUCAUU | Chmp | [1691-1709] | [1662-1680] |
| 464 | CUUCAAGGCCAUGUUCACC | GGUGAACAUGGCCUUGAAG | Chmp | [503-521] | [474-492] |
| 465 | CUCUGGAAAUGUGGUUCCC | GGGAACCACAUUUCCAGAG | Chmp | [2444-2462] | [2415-2433] |
| 466 | ACCAGCAGAACUGUACCUG | CAGGUACAGUUCUGCUGGU | Chmp | [2038-2056] | [2009-2027] |
| 467 | CCUCUGGAAAUGUGGUUCC | GGAACCACAUUUCCAGAGG | Chmp | [2443-2461] | [2414-2432] |
| 468 | GUCAAGAUCUUCGAGGAGC | GCUCCUCGAAGAUCUUGAC | Chmp | [1089-1107] | [1060-1078] |
| 469 | ACAGCAAUGAACACCAUCC | GGAUGGUGUUCAUUGCUGU | Chmp | [1686-1704] | [1657-1675] |
| 470 | AAUCUACGUCCUUGGAGGC | GCCUCCAAGGACGUAGAUU | Chmp | [1880-1898] | [1851-1869] |
| 471 | UAUGAUGGUCAGGACCAGC | GCUGGUCCUGACCAUCAUA | Chmp | [1758-1776] | [1729-1747] |
| 472 | UCCUUGGAGGCUAUGAUGG | CCAUCAUAGCCUCCAAGGA | Chmp | [1888-1906] | [1859-1877] |
| 473 | UGCAUCCACCACAACAGUG | CACUGUUGUGGUGGAUGCA | Chmp | [1485-1503] | [1456-1474] |
| 474 | UACAUCUACAUGCAUUUUG | CAAAAUGCAUGUAGAUGUA | Chmp | [801-819] | [772-790] |
| 475 | AACUUCCUGCAGAUGCAGC | GCUGCAUCUGCAGGAAGUU | Chmp | [1020-1038] | [991-1009] |
| 476 | AUUGGCUGUGUGGAGUUGC | GCAACUCCACACAGCCAAU | | [765-783] | [736-754] |
| 477 | UACCUGGAGGCUUACAACC | GGUUGUAAGCCUCCAGGUA | Chmp | [1206-1224] | [1177-1195] |
| 478 | ACACUGCAGGUCAAGUACC | GGUACUUGACCUGCAGUGU | Chmp | [423-441] | [394-412] |
| 479 | UACCUGGUCAAGAUCUUCG | CGAAGAUCUUGACCAGGUA | Chmp | [1083-1101] | [1054-1072] |
| 480 | GAGUUCUUCAACCUGUCCC | GGGACAGGUUGAAGAACUC | GP, Chmp | [840-858] | [811-829] |
| 481 | UACGUCCUUGGAGGCUAUG | CAUAGCCUCCAAGGACGUA | Chmp | [1884-1902] | [1855-1873] |
| 482 | UUCCUGGACAGUGUGGAGU | ACUCCACACUGUCCAGGAA | GP, Chmp | [1914-1932] | [1885-1903] |
| 483 | UUUGGCAUCAUGAACGAGC | GCUCGUUCAUGAUGCCAAA | Rat, Chmp | [375-393] | [346-364] |
| 484 | GACAUCUCAAAAGAAGUCC | GGACUUCUUUUGAGAUGUC | Chmp | [2203-2221] | [2174-2192] |
| 485 | AUCUACGUCCUUGGAGGCU | AGCCUCCAAGGACGUAGAU | Chmp | [1881-1899] | [1852-1870] |
| 486 | UUGGGCAAAAAUACAGUCC | GGACUGUAUUUUUGCCCAA | | [2075-2093] | [2046-2064] |
| 487 | AUGAACACCAUCCGAAGCG | CGCUUCGGAUGGUGUUCAU | Chmp | [1692-1710] | [1663-1681] |
| 488 | AUCCACCACAACAGUGUGG | CCACACUGUUGUGGUGGAU | Chmp | [1488-1506] | [1459-1477] |
| 489 | AUGUACCAGAUCGACAGCG | CGCUGUCGAUCUGGUACAU | Chn, Chmp | [666-684] | [637-655] |

TABLE B7
SHC1 19-mer oligomer pairs - SHC transforming protein 1

| | | | | Human- | |
|---|---|---|---|---|---|
| | | | Other | 52693920: | Human-34147725: |
| No. | Sense | AntiSense | Sp | 1-1752 | 121-1545 |

| | | | | | |
|---|---|---|---|---|---|
| 1 | ACCUGAAAUUUGCUGGAAU | AUUCCAGCAAAUUUCAGGU | | [671-689] | [461-479] |
| 2 | CGAGUAUGUCGCCUAUGUU | AACAUAGGCGACAUACUCG | | [813-831] | [603-621] |
| 3 | CAGAGAGCUUUUUGAUGAU | AUCAUCAAAAAGCUCUCUG | | [1254-1272] | [1047-1065] |
| 4 | GCAGAGAGCUUUUUGAUGA | UCAUCAAAAAGCUCUCUGC | | [1253-1271] | [1046-1064] |
| 5 | CCGCUUUGAAAGUGUCAGU | ACUGACACUUUCAAAGCGG | | [1641-1659] | [1434-1452] |
| 6 | AGCUUUUUGAUGAUCCCUC | GAGGGAUCAUCAAAAAGCU | | [1259-1277] | [1052-1070] |
| 7 | UGACCAUCAGUACUAUAAU | AUUAUAGUACUGAUGGUCA | | [1035-1053] | [825-843] |
| 8 | CACAUGCAAUCUAUCUCAU | AUGAGAUAGAUUGCAUGUG | Rat | [766-784] | [556-574] |
| 9 | CCUACUUGGUUCGGUACAU | AUGUACCGAACCAAGUAGG | Rat, Ms | [482-500] | [272-290] |
| 10 | CCUAUGUCAACGUCCAGAA | UUCUGGACGUUGACAUAGG | | [1277-1295] | [1070-1088] |
| 11 | AGAGAGCUUUUUGAUGAUC | GAUCAUCAAAAAGCUCUCU | | [1255-1273] | [1048-1066] |
| 12 | AGAGCUUUUUGAUGAUCCC | GGGAUCAUCAAAAAGCUCU | | [1257-1275] | [1050-1068] |
| 13 | UCAAACAAUACCUCAGGAA | UUCCUGAGGUAUUGUUUGA | | [935-953] | [725-743] |
| 14 | ACAUGCAAUCUAUCUCAUU | AAUGAGAUAGAUUGCAUGU | Rat | [767-785] | [557-575] |
| 15 | AACCUGAAAUUUGCUGGAA | UUCCAGCAAAUUUCAGGUU | | [670-688] | [460-478] |
| 16 | CGCUUUGAAAGUGUCAGUC | GACUGACACUUUCAAAGCG | | [1642-1660] | [1435-1453] |
| 17 | GGGACCUGUUUGACAUGAA | UUCAUGUCAAACAGGUCCC | | [1367-1385] | [1160-1178] |
| 18 | CCUCCUAUGUCAACGUCCA | UGGACGUUGACAUAGGAGG | | [1274-1292] | [1067-1085] |
| 19 | UCCCUCCUAUGUCAACGUC | GACGUUGACAUAGGAGGGA | | [1272-1290] | [1065-1083] |
| 20 | GUACUAUAAUGACUUCCCG | CGGGAAGUCAUUAUAGUAC | | [1044-1062] | [834-852] |
| 21 | AGGGUGUGGUUCGGACUAA | UUAGUCCGAACCACACCCU | | [1616-1634] | [1409-1427] |
| 22 | AGACAUGAGGCUUCGGGAA | UUCCCGAAGCCUCAUGUCU | | [1092-1110] | [882-900] |
| 23 | GCGCUUCAAACAAUACCUC | GAGGUAUUGUUUGAAGCGC | | [930-948] | [720-738] |
| 24 | CCAAUCACUCUCACCGUCU | AGACGGUGAGAGUGAUUGG | | [691-709] | [481-499] |
| 25 | GCAUUUGCUACUGGUGGAC | GUCCACCAGUAGCAAAUGC | | [1593-1611] | [1386-1404] |
| 26 | GUGGUAGACAUGAGGCUUC | GAAGCCUCAUGUCUACCAC | | [1087-1105] | [877-895] |
| 27 | CAAUCACUCUCACCGUCUC | GAGACGGUGAGAGUGAUUG | | [692-710] | [482-500] |
| 28 | CGGGAGCUUUGUCAAUAAG | CUUAUUGACAAAGCUCCCG | Rat, Ms | [411-429] | [201-219] |
| 29 | GGACAUGAACAAGCUGAGU | ACUCAGCUUGUUCAUGUCC | Rat, Ms | [327-345] | [117-135] |
| 30 | CCCAAUCCUGCUAUCAAUG | CAUUGAUAGCAGGAUUGGG | | [1336-1354] | [1129-1147] |
| 31 | CGAGUUGCGCUUCAAACAA | UUGUUUGAAGCGCAACUCG | | [924-942] | [714-732] |
| 32 | GCUUUGAAAGUGUCAGUCA | UGACUGACACUUUCAAAGC | | [1643-1661] | [1436-1454] |
| 33 | UGAUGGCUCAGCAUGGGAU | AUCCCAUGCUGAGCCAUCA | | [996-1014] | [786-804] |
| 34 | UGCGCUUCAAACAAUACCU | AGGUAUUGUUUGAAGCGCA | | [929-947] | [719-737] |
| 35 | AUGCAAUCUAUCUCAUUUG | CAAAUGAGAUAGAUUGCAU | | [769-787] | [559-577] |
| 36 | ACCACAUGCAAUCUAUCUC | GAGAUAGAUUGCAUGUGGU | | [764-782] | [554-572] |
| 37 | CCACCACAUGCAAUCUAUC | GAUAGAUUGCAUGUGGUGG | | [762-780] | [552-570] |
| 38 | GUACAUGGGUUGUGUGGAG | CUCCACACAACCCAUGUAC | | [495-513] | [285-303] |
| 39 | GGAUCACCGCUUUGAAAGU | ACUUUCAAAGCGGUGAUCC | | [1635-1653] | [1428-1446] |
| 40 | GGUUCGGACUAAGGAUCAC | GUGAUCCUUAGUCCGAACC | | [1623-1641] | [1416-1434] |
| 41 | CGGGACCUGUUUGACAUGA | UCAUGUCAAACAGGUCCCG | | [1366-1384] | [1159-1177] |
| 42 | CGCUUCAAACAAUACCUCA | UGAGGUAUUGUUUGAAGCG | | [931-949] | [721-739] |
| 43 | GACAUGAACAAGCUGAGUG | CACUCAGCUUGUUCAUGUC | Rat, Ms | [328-346] | [118-136] |
| 44 | GCUUCAAACAAUACCUCAG | CUGAGGUAUUGUUUGAAGC | | [932-950] | [722-740] |
| 45 | AGUUGCGCUUCAAACAAUA | UAUUGUUUGAAGCGCAACU | | [926-944] | [716-734] |
| 46 | AGCUACCACAUGGACAAUC | GAUUGUCCAUGUGGUAGCU | Rat, Ms | [1669-1687] | [1462-1480] |
| 47 | CAGCUACCACAUGGACAAU | AUUGUCCAUGUGGUAGCUG | Rat, Ms | [1668-1686] | [1461-1479] |
| 48 | ACCUUAUCAGCUACCACAU | AUGUGGUAGCUGAUAAGGU | | [1661-1679] | [1454-1472] |
| 49 | GGACUAAGGAUCACCGCUU | AAGCGGUGAUCCUUAGUCC | | [1628-1646] | [1421-1439] |
| 50 | UGGGCAGCCUAAGCAUUUG | CAAAUGCUUAGGCUGCCCA | | [1581-1599] | [1374-1392] |
| 51 | CGAACUGUGUCUACAGCAA | UUGCUGUAGACACAGUUCG | | [1713-1731] | [1506-1524] |
| 52 | GUGUCAGUCACCUUAUCAG | CUGAUAAGGUGACUGACAC | | [1652-1670] | [1445-1463] |
| 53 | GGGAGGAGUAACCUGAAAU | AUUUCAGGUUACUCCUCCC | | [661-679] | [451-469] |
| 54 | GCCUAAGCAUUUGCUACUG | CAGUAGCAAAUGCUUAGGC | | [1587-1605] | [1380-1398] |
| 55 | ACAAUCACUUGCCCAUCAU | AUGAUGGGCAAGUGAUUGU | Ms | [1682-1700] | [1475-1493] |
| 56 | AGAUCCAGAAGUCCGCAAA | UUUGCGGACUUCUGGAUCU | | [1206-1224] | [996-1014] |
| 57 | CCAUCAGUACUAUAAUGAC | GUCAUUAUAGUACUGAUGG | | [1038-1056] | [828-846] |
| 58 | AGCCACCUGACCAUCAGUA | UACUGAUGGUCAGGUGGCU | | [1028-1046] | [818-836] |
| 59 | AGCCGAGUAUGUCGCCUAU | AUAGGCGACAUACUCGGCU | | [810-828] | [600-618] |
| 60 | AGGAGGAGAAAGCCCUGUA | UACAGGGCUUUCUCCUCCU | | [619-637] | [409-427] |
| 61 | UCAGCUACCACAUGGACAA | UUGUCCAUGUGGUAGCUGA | Rat, Ms | [1667-1685] | [1460-1478] |
| 62 | GGGAGCUACAUUGCCUGUA | UACAGGCAAUGUAGCUCCC | | [1170-1188] | [960-978] |
| 63 | CUGACCAUCAGUACUAUAA | UUAUAGUACUGAUGGUCAG | | [1034-1052] | [824-842] |
| 64 | GAAAUUUGCUGGAAUGCCA | UGGCAUUCCAGCAAAUUUC | | [675-693] | [465-483] |
| 65 | ACGUCCAGAACCUAGACAA | UUGUCUAGGUUCUGGACGU | | [1286-1304] | [1079-1097] |
| 66 | ACCAUCAGUACUAUAAUGA | UCAUUAUAGUACUGAUGGU | | [1037-1055] | [827-845] |
| 67 | GCAAACAGAUCAUCGCCAA | UUGGCGAUGAUCUGUUUGC | | [743-761] | [533-551] |
| 68 | CAGACUGCAAACAGAUCAU | AUGAUCUGUUUGCAGUCUG | Rat | [737-755] | [527-545] |
| 69 | ACCACAUGGACAAUCACUU | AAGUGAUUGUCCAUGUGGU | Rat, Ms | [1673-1691] | [1466-1484] |
| 70 | CUAAGCAUUUGCUACUGGU | ACCAGUAGCAAAUGCUUAG | | [1589-1607] | [1382-1400] |
| 71 | GAGAUCCAGAAGUCCGCAA | UUGCGGACUUCUGGAUCUC | | [1205-1223] | [995-1013] |
| 72 | CGCCUAUGUUGCCAAAGAC | GUCUUUGGCAACAUAGGCG | | [822-840] | [612-630] |
| 73 | UGGACAAUCACUUGCCCAU | AUGGGCAAGUGAUUGUCCA | Ms | [1679-1697] | [1472-1490] |
| 74 | AGGCAGAGAGCUUUUUGAU | AUCAAAAAGCUCUCUGCCU | | [1251-1269] | [1044-1062] |
| 75 | CCUGACCAUCAGUACUAUA | UAUAGUACUGAUGGUCAGG | | [1033-1051] | [823-841] |
| 76 | CCGCAGACUGCAAACAGAU | AUCUGUUUGCAGUCUGCGG | | [734-752] | [524-542] |
| 77 | GGGUUUCCUACUUGGUUCG | CGAACCAAGUAGGAAACCC | Rat, Ms | [476-494] | [266-284] |
| 78 | GGACAAUCACUUGCCCAUC | GAUGGGCAAGUGAUUGUCC | Ms | [1680-1698] | [1473-1491] |
| 79 | GCAGCCUAAGCAUUUGCUA | UAGCAAAUGCUUAGGCUGC | | [1584-1602] | [1377-1395] |
| 80 | CAGGCAGAGAGCUUUUUGA | UCAAAAAGCUCUCUGCCUG | | [1250-1268] | [1043-1061] |
| 81 | GGAGCUACAUUGCCUGUAG | CUACAGGCAAUGUAGCUCC | | [1171-1189] | [961-979] |
| 82 | GCAAUCUAUCUCAUUUGCA | UGCAAAUGAGAUAGAUUGC | | [771-789] | [561-579] |
| 83 | UGCAUCCCAACGACAAAGU | ACUUUGUCGUUGGGAUGCA | Rat, Ms | [446-464] | [236-254] |
| 84 | GAAUGCCAAUCACUCUCAC | GUGAGAGUGAUUGGCAUUC | Ms | [686-704] | [476-494] |
| 85 | CAACGACAAAGUCAUGGGA | UCCCAUGACUUUGUCGUUG | Rat, Ms | [453-471] | [243-261] |
| 86 | AGCCUAAGCAUUUGCUACU | AGUAGCAAAUGCUUAGGCU | | [1586-1604] | [1379-1397] |
| 87 | GGCAGCCUAAGCAUUUGCU | AGCAAAUGCUUAGGCUGCC | | [1583-1601] | [1376-1394] |
| 88 | ACCUGACCAUCAGUACUAU | AUAGUACUGAUGGUCAGGU | | [1032-1050] | [822-840] |
| 89 | CACCUGACCAUCAGUACUA | UAGUACUGAUGGUCAGGUG | | [1031-1049] | [821-839] |
| 90 | GGCUUUGAUGGCUCAGCAU | AUGCUGAGCCAUCAAAGCC | | [991-1009] | [781-799] |
| 91 | ACCACCACAUGCAAUCUAU | AUAGAUUGCAUGUGGUGGU | | [761-779] | [551-569] |
| 92 | GCAGACUGCAAACAGAUCA | UGAUCUGUUUGCAGUCUGC | Rat | [736-754] | [526-544] |
| 93 | UGAAAGUGUCAGUCACCUU | AAGGUGACUGACACUUUCA | | [1647-1665] | [1440-1458] |
| 94 | GGCAGAGAGCUUUUUGAUG | CAUCAAAAAGCUCUCUGCC | | [1252-1270] | [1045-1063] |
| 95 | GCCACUUGGGAGCUACAUU | AAUGUAGCUCCCAAGUGGC | | [1163-1181] | [953-971] |
| 96 | UGUCGCCUAUGUUGCCAAA | UUUGGCAACAUAGGCGACA | | [819-837] | [609-627] |
| 97 | UGGAGGUCCUCCAGUCAAU | AUUGACUGGAGGACCUCCA | | [509-527] | [299-317] |
| 98 | GUCACCUUAUCAGCUACCA | UGGUAGCUGAUAAGGUGAC | | [1658-1676] | [1451-1469] |
| 99 | UCACCGCUUUGAAAGUGUC | GACACUUUCAAAGCGGUGA | | [1638-1656] | [1431-1449] |
| 100 | CCUAAGCAUUUGCUACUGG | CCAGUAGCAAAUGCUUAGG | | [1588-1606] | [1381-1399] |
| 101 | GCCCUUCGAAGAUGCUCUU | AAGAGCAUCUUCGAAGGGC | | [1386-1404] | [1179-1197] |
| 102 | UGCUAUCAAUGGCAGUGCA | UGCACUGCCAUUGAUAGCA | | [1344-1362] | [1137-1155] |
| 103 | UGACAGGAUGGCUGGCUUU | AAAGCCAGCCAUCCUGUCA | Rat, Ms | [978-996] | [768-786] |
| 104 | CCUUCGAGUUGCGCUUCAA | UUGAAGCGCAACUCGAAGG | | [920-938] | [710-728] |
| 105 | UUGCCAAAGACCCUGUGAA | UUCACAGGGUCUUUGGCAA | Rat, Ms | [830-848] | [620-638] |
| 106 | UGCAAUCUAUCUCAUUUGC | GCAAAUGAGAUAGAUUGCA | | [770-788] | [560-578] |
| 107 | GCGAACUGUGUCUACAGCA | UGCUGUAGACACAGUUCGC | | [1712-1730] | [1505-1523] |
| 108 | AGUAUGUGCUCACUGGCUU | AAGCCAGUGAGCACAUACU | | [1556-1574] | [1349-1367] |
| 109 | UGGCUUUGAUGGCUCAGCA | UGCUGAGCCAUCAAAGCCA | | [990-1008] | [780-798] |
| 110 | AGACUGCAAACAGAUCAUC | GAUGAUCUGUUUGCAGUCU | | [738-756] | [528-546] |
| 111 | GCAUCCCAACGACAAAGUC | GACUUUGUCGUUGGGAUGC | Rat, Ms | [447-465] | [237-255] |
| 112 | GCUGCAUCCCAACGACAAA | UUUGUCGUUGGGAUGCAGC | Rat, Ms | [444-462] | [234-252] |
| 113 | AGCAUUUGCUACUGGUGGA | UCCACCAGUAGCAAAUGCU | | [1592-1610] | [1385-1403] |
| 114 | UCAACGUCCAGAACCUAGA | UCUAGGUUCUGGACGUUGA | | [1283-1301] | [1076-1094] |
| 115 | UGCCAAAGACCCUGUGAAU | AUUCACAGGGUCUUUGGCA | Rat, Ms | [831-849] | [621-639] |
| 116 | CCAACCACCACAUGCAAUC | GAUUGCAUGUGGUGGUUGG | | [758-776] | [548-566] |
| 117 | UGCAAACAGAUCAUCGCCA | UGGCGAUGAUCUGUUUGCA | | [742-760] | [532-550] |
| 118 | CCAACGACAAAGUCAUGGG | CCCAUGACUUUGUCGUUGG | Rat, Ms | [452-470] | [242-260] |
| 119 | GGAGCUUUGUCAAUAAGCC | GGCUUAUUGACAAAGCUCC | Rat, Ms | [413-431] | [203-221] |
| 120 | CCUGCUAUCAAUGGCAGUG | CACUGCCAUUGAUAGCAGG | | [1342-1360] | [1135-1153] |
| 121 | AGCCACUUGGGAGCUACAU | AUGUAGCUCCCAAGUGGCU | | [1162-1180] | [952-970] |
| 122 | CCACCUGACCAUCAGUACU | AGUACUGAUGGUCAGGUGG | | [1030-1048] | [820-838] |
| 123 | GCCAAAGACCCUGUGAAUC | GAUUCACAGGGUCUUUGGC | Rat, Ms | [832-850] | [622-640] |
| 124 | AACCACCACAUGCAAUCUA | UAGAUUGCAUGUGGUGGUU | | [760-778] | [550-568] |
| 125 | GCUUUGUCAAUAAGCCCAC | GUGGGCUUAUUGACAAAGC | Rat, Ms | [416-434] | [206-224] |
| 126 | CAGUCACCUUAUCAGCUAC | GUAGCUGAUAAGGUGACUG | | [1656-1674] | [1449-1467] |
| 127 | UGACAUGAAGCCCUUCGAA | UUCGAAGGGCUUCAUGUCA | Rat | [1377-1395] | [1170-1188] |
| 128 | CUGCCACAUUCUGGAGUGU | ACACUCCAGAAUGUGGCAG | | [858-876] | [648-666] |
| 129 | GCCAACCACCACAUGCAAU | AUUGCAUGUGGUGGUUGGC | | [757-775] | [547-565] |
| 130 | CGCAGACUGCAAACAGAUC | GAUCUGUUUGCAGUCUGCG | | [735-753] | [525-543] |
| 131 | CACCUUAUCAGCUACCACA | UGUGGUAGCUGAUAAGGUG | | [1660-1678] | [1453-1471] |
| 132 | CAGGAUGUCAUCAGCACCA | UGGUGCUGAUGACAUCCUG | Ms | [892-910] | [682-700] |
| 133 | UGGCCAGUAUGUGCUCACU | AGUGAGCACAUACUGGCCA | Ms | [1551-1569] | [1344-1362] |
| 134 | AGGAUGUCAUCAGCACCAU | AUGGUGCUGAUGACAUCCU | Ms | [893-911] | [683-701] |
| 135 | CCCUUCGAAGAUGCUCUUC | GAAGAGCAUCUUCGAAGGG | | [1387-1405] | [1180-1198] |
| 136 | UGUUUGACAUGAAGCCCUU | AAGGGCUUCAUGUCAAACA | | [1373-1391] | [1166-1184] |
| 137 | CUGUUUGACAUGAAGCCCU | AGGGCUUCAUGUCAAACAG | | [1372-1390] | [1165-1183] |
| 138 | GGAUGUCAUCAGCACCAUU | AAUGGUGCUGAUGACAUCC | | [894-912] | [684-702] |
| 139 | UCAUCGCCAACCACCACAU | AUGUGGUGGUUGGCGAUGA | | [752-770] | [542-560] |
| 140 | GGGUUCUUAUAAUGGAAAA | UUUUCCAUUAUAAGAACCC | | | |
| 141 | CCCAAGCCCAAGUACAAUC | GAUUGUACUUGGGCUUGGG | | | |
| 142 | AGGAAGGGCAGCUGAUGAU | AUCAUCAGCUGCCCUUCCU | | | |

TABLE B8
SHC1 - 19-mer oligomer pairs SHC transforming protein 1

| | | | | Human- | Human- |
|---|---|---|---|---|---|
| | | | | 52693920: | 34147725: |
| No. | Sense | AntiSense | Other Sp | 1-1752 | 121-1545 |

| | | | | | |
|---|---|---|---|---|---|
| 1 | ACGGGAGCUUUGUCAAUAA | UUAUUGACAAAGCUCCCGU | Rat, Ms, GP, | [410-428] | [200-218] |
| | | | Chn | | |
| 2 | CAGGAUGGCUGGCUUUGAU | AUCAAAGCCAGCCAUCCUG | Rat, Ms | [981-999] | [771-789] |
| 3 | CCAGAAGUCCGCAAACAGA | UCUGUUUGCGGACUUCUGG | | [1210-1228] | [1000-1018] |
| 4 | CAAUCUAUCUCAUUUGCAU | AUGCAAAUGAGAUAGAUUG | | [772-790] | [562-580] |
| 5 | GAUGAUCCCUCCUAUGUCA | UGACAUAGGAGGGAUCAUC | | [1267-1285] | [1060-1078] |
| 6 | GGAGGAGUAACCUGAAAUU | AAUUUCAGGUUACUCCUCC | GP, Chn | [662-680] | [452-470] |
| 7 | UAAGGAUCACCGCUUUGAA | UUCAAAGCGGUGAUCCUUA | | [1632-1650] | [1425-1443] |
| 8 | GAGUUGCGCUUCAAACAAU | AUUGUUUGAAGCGCAACUC | | [925-943] | [715-733] |
| 9 | AAAUUUGCUGGAAUGCCAA | UUGGCAUUCCAGCAAAUUU | | [676-694] | [466-484] |
| 10 | GUGUGGUUCGGACUAAGGA | UCCUUAGUCCGAACCACAC | | [1619-1637] | [1412-1430] |
| 11 | AAGGAUCACCGCUUUGAAA | UUUCAAAGCGGUGAUCCUU | | [1633-1651] | [1426-1444] |
| 12 | UUUUGAUGAUCCCUCCUAU | AUAGGAGGGAUCAUCAAAA | | [1263-1281] | [1056-1074] |
| 13 | UGUGGUUCGGACUAAGGAU | AUCCUUAGUCCGAACCACA | | [1620-1638] | [1413-1431] |
| 14 | UGUGGAGCGGAAACUGUGA | UCACAGUUUCCGCUCCACA | | [1734-1752] | [1527-1545] |
| 15 | CUUCGAGUUGCGCUUCAAA | UUUGAAGCGCAACUCGAAG | | [921-939] | [711-729] |
| 16 | GGACCUGUUUGACAUGAAG | CUUCAUGUCAAACAGGUCC | | [1368-1386] | [1161-1179] |
| 17 | GGAGUAACCUGAAAUUUGC | GCAAAUUUCAGGUUACUCC | GP | [665-683] | [455-473] |
| 18 | GCUACCACAUGGACAAUCA | UGAUUGUCCAUGUGGUAGC | Rat, Ms | [1670-1688] | [1463-1481] |
| 19 | AUGGGAUGAGGAGGAGGAA | UUCCUCCUCCUCAUCCCAU | | [1008-1026] | [798-816] |
| 20 | CCACGGGAGCUUUGUCAAU | AUUGACAAAGCUCCCGUGG | | [408-426] | [198-216] |
| 21 | CAUUCUGGAGUGUCCCGAA | UUCGGGACACUCCAGAAUG | | [864-882] | [654-672] |
| 22 | GACUAAGGAUCACCGCUUU | AAAGCGGUGAUCCUUAGUC | | [1629-1647] | [1422-1440] |
| 23 | ACAUGAAGCCCUUCGAAGA | UCUUCGAAGGGCUUCAUGU | Rat | [1379-1397] | [1172-1190] |
| 24 | CACGGGAGCUUUGUCAAUA | UAUUGACAAAGCUCCCGUG | Rat, Ms, GP, | [409-427] | [199-217] |
| | | | Chn | | |
| 25 | CCAUCAGUCUGGUGUGUGA | UCACACACCAGACUGAUGG | GP, Chn | [572-590] | [362-380] |
| 26 | UAGACAUGAGGCUUCGGGA | UCCCGAAGCCUCAUGUCUA | Chn | [1091-1109] | [881-899] |
| 27 | CCACAUGCAAUCUAUCUCA | UGAGAUAGAUUGCAUGUGG | | [765-783] | [555-573] |
| 28 | CAUGAACAAGCUGAGUGGA | UCCACUCAGCUUGUUCAUG | Rat, Ms, GP, Chn | [330-348] | [120-138] |
| 29 | GGUACAUGGGUUGUGUGGA | UCCACACAACCCAUGUACC | | [494-512] | [284-302] |
| 30 | CCUGAAAUUUGCUGGAAUG | CAUUCCAGCAAAUUUCAGG | | [672-690] | [462-480] |
| 31 | GAGUAACCUGAAAUUUGCU | AGCAAAUUUCAGGUUACUC | | [666-684] | [456-474] |
| 32 | GAAAGUGUCAGUCACCUUA | UAAGGUGACUGACACUUUC | | [1648-1666] | [1441-1459] |
| 33 | CAGAAGUCCGCAAACAGAU | AUCUGUUUGCGGACUUCUG | | [1211-1229] | [1001-1019] |
| 34 | CCGAGUAUGUCGCCUAUGU | ACAUAGGCGACAUACUCGG | Chn | [812-830] | [602-620] |
| 35 | AGAUCAUCGCCAACCACCA | UGGUGGUUGGCGAUGAUCU | | [749-767] | [539-557] |
| 36 | GAGUAUGUCGCCUAUGUUG | CAACAUAGGCGACAUACUC | Chn | [814-832] | [604-622] |
| 37 | UCGAGUUGCGCUUCAAACA | UGUUUGAAGCGCAACUCGA | | [923-941] | [713-731] |
| 38 | UUUCCUACUUGGUUCGGUA | UACCGAACCAAGUAGGAAA | Rat, Ms, GP, Chn | [479-497] | [269-287] |
| 39 | AAUCAGAGAGCCUGCCACA | UGUGGCAGGCUCUCUGAUU | Chn | [847-865] | [637-655] |
| 40 | CAUGCAAUCUAUCUCAUUU | AAAUGAGAUAGAUUGCAUG | | [768-786] | [558-576] |
| 41 | CUUGGUUCGGUACAUGGGU | ACCCAUGUACCGAACCAAG | Rat | [486-504] | [276-294] |
| 42 | CAAACAGAUGCCACCUCCA | UGGAGGUGGCAUCUGUUUG | | [1221-1239] | [1011-1029] |
| 43 | CGUCCAGAACCUAGACAAG | CUUGUCUAGGUUCUGGACG | GP | [1287-1305] | [1080-1098] |
| 44 | CAAACAAUACCUCAGGAAC | GUUCCUGAGGUAUUGUUUG | | [936-954] | [726-744] |
| 45 | UGUCAACGUCCAGAACCUA | UAGGUUCUGGACGUUGACA | GP | [1281-1299] | [1074-1092] |
| 46 | UCGCCUAUGUUGCCAAAGA | UCUUUGGCAACAUAGGCGA | Chn | [821-839] | [611-629] |
| 47 | CAAACAGAUCAUCGCCAAC | GUUGGCGAUGAUCUGUUUG | | [744-762] | [534-552] |
| 48 | CCAAAGACCCUGUGAAUCA | UGAUUCACAGGGUCUUUGG | Rat, Ms, GP, Chn | [833-851] | [623-641] |
| 49 | CAUGAAGCCCUUCGAAGAU | AUCUUCGAAGGGCUUCAUG | Rat | [1380-1398] | [1173-1191] |
| 50 | GCUUUUUGAUGAUCCCUCC | GGAGGGAUCAUCAAAAAGC | | [1260-1278] | [1053-1071] |
| 51 | AACAGAUCAUCGCCAACCA | UGGUUGGCGAUGAUCUGUU | | [746-764] | [536-554] |
| 52 | UAACCUGAAAUUUGCUGGA | UCCAGCAAAUUUCAGGUUA | | [669-687] | [459-477] |
| 53 | GCAGCGAACUGUGUCUACA | UGUAGACACAGUUCGCUGC | | [1709-1727] | [1502-1520] |
| 54 | UCAGUCACCUUAUCAGCUA | UAGCUGAUAAGGUGACUGA | | [1655-1673] | [1448-1466] |
| 55 | UGGUUCGGACUAAGGAUCA | UGAUCCUUAGUCCGAACCA | | [1622-1640] | [1415-1433] |
| 56 | CUAUGUUGCCAAAGACCCU | AGGGUCUUUGGCAACAUAG | Rat, Ms, GP, Chn | [825-843] | [615-633] |
| 57 | AGACCCUGUGAAUCAGAGA | UCUCUGAUUCACAGGGUCU | Rat, Ms, GP, Chn | [837-855] | [627-645] |
| 58 | CAAAGACCCUGUGAAUCAG | CUGAUUCACAGGGUCUUUG | Rat, Ms, GP, Chn | [834-852] | [624-642] |
| 59 | CUAUGUCAACGUCCAGAAC | GUUCUGGACGUUGACAUAG | GP | [1278-1296] | [1071-1089] |
| 60 | CUACCACAUGGACAAUCAC | GUGAUUGUCCAUGUGGUAG | Rat, Ms | [1671-1689] | [1464-1482] |
| 61 | ACUUCAACACCCGGACUCA | UGAGUCCGGGUGUUGAAGU | | [539-557] | [329-347] |
| 62 | GGUGGUAGACAUGAGGCUU | AAGCCUCAUGUCUACCACC | GP | [1086-1104] | [876-894] |
| 63 | AUGAUCCCUCCUAUGUCAA | UUGACAUAGGAGGGAUCAU | | [1268-1286] | [1061-1079] |
| 64 | GGUUCGGUACAUGGGUUGU | ACAACCCAUGUACCGAACC | | [489-507] | [279-297] |
| 65 | CUACUUGGUUCGGUACAUG | CAUGUACCGAACCAAGUAG | Rat, Ms, GP, Chn | [483-501] | [273-291] |
| 66 | CUACAUUGCCUGUAGGACA | UGUCCUACAGGCAAUGUAG | | [1175-1193] | [965-983] |
| 67 | GAUCCAGAAGUCCGCAAAC | GUUUGCGGACUUCUGGAUC | | [1207-1225] | [997-1015] |
| 68 | ACAGGAUGGCUGGCUUUGA | UCAAAGCCAGCCAUCCUGU | Rat, Ms | [980-998] | [770-788] |
| 69 | GGAAUGCCAAUCACUCUCA | UGAGAGUGAUUGGCAUUCC | Ms, Chn | [685-703] | [475-493] |
| 70 | CAUCAGUACUAUAAUGACU | AGUCAUUAUAGUACUGAUG | GP, Chn | [1039-1057] | [829-847] |
| 71 | CUUUGAAAGUGUCAGUCAC | GUGACUGACACUUUCAAAG | | [1644-1662] | [1437-1455] |
| 72 | CUGGAAUGCCAAUCACUCU | AGAGUGAUUGGCAUUCCAG | Ms, Chn | [683-701] | [473-491] |
| 73 | GUGGGCAGCCUAAGCAUUU | AAAUGCUUAGGCUGCCCAC | | [1580-1598] | [1373-1391] |
| 74 | AUGUCAACGUCCAGAACCU | AGGUUCUGGACGUUGACAU | GP | [1280-1298] | [1073-1091] |
| 75 | ACAAUACCUCAGGAACCCA | UGGGUUCCUGAGGUAUUGU | GP | [939-957] | [729-747] |
| 76 | CUAAGGAUCACCGCUUUGA | UCAAAGCGGUGAUCCUUAG | | [1631-1649] | [1424-1442] |
| 77 | AGUGGGCAGCCUAAGCAUU | AAUGCUUAGGCUGCCCACU | | [1579-1597] | [1372-1390] |
| 78 | AGUGUCAGUCACCUUAUCA | UGAUAAGGUGACUGACACU | | [1651-1669] | [1444-1462] |
| 79 | UUGACAUGAAGCCCUUCGA | UCGAAGGGCUUCAUGUCAA | Rat | [1376-1394] | [1169-1187] |
| 80 | AUGUCGCCUAUGUUGCCAA | UUGGCAACAUAGGCGACAU | Chn | [818-836] | [608-626] |
| 81 | CACCGCUUUGAAAGUGUCA | UGACACUUUCAAAGCGGUG | | [1639-1657] | [1432-1450] |
| 82 | UGAUGAUCCCUCCUAUGUC | GACAUAGGAGGGAUCAUCA | | [1266-1284] | [1059-1077] |
| 83 | ACAUGAACAAGCUGAGUGG | CCACUCAGCUUGUUCAUGU | Rat, Ms, GP, Chn | [329-347] | [119-137] |
| 84 | CAAUACCUCAGGAACCCAC | GUGGGUUCCUGAGGUAUUG | GP | [940-958] | [730-748] |
| 85 | CCACUUGGGAGCUACAUUG | CAAUGUAGCUCCCAAGUGG | | [1164-1182] | [954-972] |
| 86 | UUCAAACAAUACCUCAGGA | UCCUGAGGUAUUGUUUGAA | | [934-952] | [724-742] |
| 87 | AAAGUGUCAGUCACCUUAU | AUAAGGUGACUGACACUUU | | [1649-1667] | [1442-1460] |
| 88 | GACAAUCACUUGCCCAUCA | UGAUGGGCAAGUGAUUGUC | Ms | [1681-1699] | [1474-1492] |
| 89 | AAAGACCCUGUGAAUCAGA | UCUGAUUCACAGGGUCUUU | Rat, Ms, GP, Chn | [835-853] | [625-643] |
| 90 | CUCCUAUGUCAACGUCCAG | CUGGACGUUGACAUAGGAG | GP | [1275-1293] | [1068-1086] |
| 91 | AUGACAGGAUGGCUGGCUU | AAGCCAGCCAUCCUGUCAU | Rat, Ms | [977-995] | [767-785] |
| 92 | UGAACAAGCUGAGUGGAGG | CCUCCACUCAGCUUGUUCA | Rat, Ms, GP, Chn | [332-350] | [122-140] |
| 93 | CUUUUUGAUGAUCCCUCCU | AGGAGGGAUCAUCAAAAAG | | [1261-1279] | [1054-1072] |
| 94 | UCCUAUGUCAACGUCCAGA | UCUGGACGUUGACAUAGGA | GP | [1276-1294] | [1069-1087] |
| 95 | GAGCUUUUUGAUGAUCCCU | AGGGAUCAUCAAAAAGCUC | | [1258-1276] | [1051-1069] |
| 96 | GAGGAGUAACCUGAAAUUU | AAAUUUCAGGUUACUCCUC | GP | [663-681] | [453-471] |
| 97 | GAACUGUGUCUACAGCAAC | GUUGCUGUAGACACAGUUC | | [1714-1732] | [1507-1525] |
| 98 | UGAUCCCUCCUAUGUCAAC | GUUGACAUAGGAGGGAUCA | | [1269-1287] | [1062-1080] |
| 99 | UCCAGAAGUCCGCAAACAG | CUGUUUGCGGACUUCUGGA | | [1209-1227] | [999-1017] |
| 100 | CAUCCCAACGACAAAGUCA | UGACUUUGUCGUUGGGAUG | Rat, Ms, GP, Chn | [448-466] | [238-256] |
| 101 | AGCUACAUUGCCUGUAGGA | UCCUACAGGCAAUGUAGCU | | [1173-1191] | [963-981] |
| 102 | GUUGCGCUUCAAACAAUAC | GUAUUGUUUGAAGCGCAAC | | [927-945] | [717-735] |
| 103 | AAGAGCCACCUGACCAUCA | UGAUGGUCAGGUGGCUCUU | GP | [1025-1043] | [815-833] |
| 104 | AGAAGUCCGCAAACAGAUG | CAUCUGUUUGCGGACUUCU | | [1212-1230] | [1002-1020] |
| 105 | CAGCCUAAGCAUUUGCUAC | GUAGCAAAUGCUUAGGCUG | | [1585-1603] | [1378-1396] |
| 106 | CAGCGAACUGUGUCUACAG | CUGUAGACACAGUUCGCUG | | [1710-1728] | [1503-1521] |
| 107 | UUGGUUCGGUACAUGGGUU | AACCCAUGUACCGAACCAA | Rat | [487-505] | [277-295] |
| 108 | CCACAUGGACAAUCACUUG | CAAGUGAUUGUCCAUGUGG | Ms | [1674-1692] | [1467-1485] |
| 109 | GACAUGAAGCCCUUCGAAG | CUUCGAAGGGCUUCAUGUC | Rat | [1378-1396] | [1171-1189] |
| 110 | AAGUGUCAGUCACCUUAUC | GAUAAGGUGACUGACACUU | | [1650-1668] | [1443-1461] |
| 111 | AGGAUGGCUGGCUUUGAUG | CAUCAAAGCCAGCCAUCCU | Rat, Ms | [982-1000] | [772-790] |
| 112 | GUUGCCAAAGACCCUGUGA | UCACAGGGUCUUUGGCAAC | Rat, Ms, GP, Chn | [829-847] | [619-637] |
| 113 | GCUGGAAUGCCAAUCACUC | GAGUGAUUGGCAUUCCAGC | Ms, Chn | [682-700] | [472-490] |
| 114 | UGCUGGAAUGCCAAUCACU | AGUGAUUGGCAUUCCAGCA | Ms | [681-699] | [471-489] |
| 115 | AGGAGUAACCUGAAAUUUG | CAAAUUUCAGGUUACUCCU | GP | [664-682] | [454-472] |
| 116 | CCUUAUCAGCUACCACAUG | CAUGUGGUAGCUGAUAAGG | | [1662-1680] | [1455-1473] |
| 117 | GCUAUCAAUGGCAGUGCAC | GUGCACUGCCAUUGAUAGC | | [1345-1363] | [1138-1156] |
| 118 | CUAUCUCAUUUGCAUCCGG | CCGGAUGCAAAUGAGAUAG | | [776-794] | [566-584] |
| 119 | UGGUUCCAUGGGAAGCUGA | UCAGCUUCCCAUGGAACCA | | [1462-1480] | [1255-1273] |
| 120 | UUUUUGAUGAUCCCUCCUA | UAGGAGGGAUCAUCAAAAA | | [1262-1280] | [1055-1073] |
| 121 | AUCAGAGAGCCUGCCACAU | AUGUGGCAGGCUCUCUGAU | Chn | [848-866] | [638-656] |
| 122 | UCAGGAACCCACCCAAACU | AGUUUGGGUGGGUUCCUGA | | [947-965] | [737-755] |
| 123 | AAUUUGCUGGAAUGCCAAU | AUUGGCAUUCCAGCAAAUU | | [677-695] | [467-485] |
| 124 | ACAUGGGUUGUGUGGAGGU | ACCUCCACACAACCCAUGU | | [497-515] | [287-305] |
| 125 | AUCAGCUACCACAUGGACA | UGUCCAUGUGGUAGCUGAU | Rat, Ms | [1666-1684] | [1459-1477] |
| 126 | CUUUGAUGGCUCAGCAUGG | CCAUGCUGAGCCAUCAAAG | | [993-1011] | [783-801] |
| 127 | UGAAUCAGAGAGCCUGCCA | UGGCAGGCUCUCUGAUUCA | Rat, Ms, Chn | [845-863] | [635-653] |
| 128 | CACCACAUGCAAUCUAUCU | AGAUAGAUUGCAUGUGGUG | | [763-781] | [553-571] |
| 129 | ACAGAUCAUCGCCAACCAC | GUGGUUGGCGAUGAUCUGU | | [747-765] | [537-555] |
| 130 | ACUUGGUUCGGUACAUGGG | CCCAUGUACCGAACCAAGU | Rat, Ms, GP, Chn | [485-503] | [275-293] |
| 131 | UUUGAUGAUCCCUCCUAUG | CAUAGGAGGGAUCAUCAAA | | [1264-1282] | [1057-1075] |
| 132 | UUAUCAGCUACCACAUGGA | UCCAUGUGGUAGCUGAUAA | | [1664-1682] | [1457-1475] |
| 133 | ACAAGGAGGAGAAAGCCCU | AGGGCUUUCUCCUCCUUGU | | [616-634] | [406-424] |
| 134 | GUCAGUCACCUUAUCAGCU | AGCUGAUAAGGUGACUGAC | | [1654-1672] | [1447-1465] |
| 135 | UGCCAAUCACUCUCACCGU | ACGGUGAGAGUGAUUGGCA | | [689-707] | [479-497] |
| 136 | AUCCAGAAGUCCGCAAACA | UGUUUGCGGACUUCUGGAU | | [1208-1226] | [998-1016] |
| 137 | UCAUCAGCACCAUUGGCCA | UGGCCAAUGGUGCUGAUGA | GP, Chn | [899-917] | [689-707] |
| 138 | AUCAGUACUAUAAUGACUU | AAGUCAUUAUAGUACUGAU | GP, Chn | [1040-1058] | [830-848] |
| 139 | GUAUGUGCUCACUGGCUUG | CAAGCCAGUGAGCACAUAC | GP | [1557-1575] | [1350-1368] |
| 140 | GAGAGCUUUUUGAUGAUCC | GGAUCAUCAAAAAGCUCUC | | [1256-1274] | [1049-1067] |
| 141 | GUUUCCUACUUGGUUCGGU | ACCGAACCAAGUAGGAAAC | Rat, Ms, GP, Chn | [478-496] | [268-286] |
| 142 | CAACGUCCAGAACCUAGAC | GUCUAGGUUCUGGACGUUG | GP | [1284-1302] | [1077-1095] |
| 143 | GCCUAUGUUGCCAAAGACC | GGUCUUUGGCAACAUAGGC | Rat, Ms, GP, Chn | [823-841] | [613-631] |
| 144 | CAAUCACUUGCCCAUCAUC | GAUGAUGGGCAAGUGAUUG | Ms | [1683-1701] | [1476-1494] |
| 145 | UCAGAGAGCCUGCCACAUU | AAUGUGGCAGGCUCUCUGA | | [849-867] | [639-657] |
| 146 | UUCGGUACAUGGGUUGUGU | ACACAACCCAUGUACCGAA | | [491-509] | [281-299] |
| 147 | UCAGUACUAUAAUGACUUC | GAAGUCAUUAUAGUACUGA | GP, Chn | [1041-1059] | [831-849] |
| 148 | AUCCCAACGACAAAGUCAU | AUGACUUUGUCGUUGGGAU | Rat, Ms, GP, Chn | [449-467] | [239-257] |
| 149 | GACCAUCAGUACUAUAAUG | CAUUAUAGUACUGAUGGUC | GP, Chn | [1036-1054] | [826-844] |
| 150 | GUAUGUCGCCUAUGUUGCC | GGCAACAUAGGCGACAUAC | Chn | [816-834] | [606-624] |
| 151 | AAACAAUACCUCAGGAACC | GGUUCCUGAGGUAUUGUUU | GP | [937-955] | [727-745] |
| 152 | GACCCUGUGAAUCAGAGAG | CUCUCUGAUUCACAGGGUC | Rat, Ms, GP, Chn | [838-856] | [628-646] |
| 153 | AGUCCGCAAACAGAUGCCA | UGGCAUCUGUUUGCGGACU | | [1215-1233] | [1005-1023] |
| 154 | CUUUGUCAAUAAGCCCACG | CGUGGGCUUAUUGACAAAG | GP, Chn | [417-435] | [207-225] |
| 155 | AAUCACUUGCCCAUCAUCU | AGAUGAUGGGCAAGUGAUU | Ms | [1684-1702] | [1477-1495] |
| 156 | GUGGUUCGGACUAAGGAUC | GAUCCUUAGUCCGAACCAC | | [1621-1639] | [1414-1432] |
| 157 | UCCUACUUGGUUCGGUACA | UGUACCGAACCAAGUAGGA | Rat, Ms, GPChn | [481-499] | [271-289] |
| 158 | UCGGUACAUGGGUUGUGUG | CACACAACCCAUGUACCGA | | [492-510] | [282-300] |
| 159 | UACUUGGUUCGGUACAUGG | CCAUGUACCGAACCAAGUA | Rat, Ms, GPChn | [484-502] | [274-292] |
| 160 | CAGUAUGUGCUCACUGGCU | AGCCAGUGAGCACAUACUG | GP | [1555-1573] | [1348-1366] |
| 161 | CUUCAAACAAUACCUCAGG | CCUGAGGUAUUGUUUGAAG | | [933-951] | [723-741] |
| 162 | AGAGAGCCUGCCACAUUCU | AGAAUGUGGCAGGCUCUCU | | [851-869] | [641-659] |
| 163 | CUGCAUCCCAACGACAAAG | CUUUGUCGUUGGGAUGCAG | Rat, Ms | [445-463] | [235-253] |
| 164 | CUUGGGAGCUACAUUGCCU | AGGCAAUGUAGCUCCCAAG | | [1167-1185] | [957-975] |
| 165 | UGGUAGACAUGAGGCUUCG | CGAAGCCUCAUGUCUACCA | GP | [1088-1106] | [878-896] |
| 166 | UCACUUGCCCAUCAUCUCU | AGAGAUGAUGGGCAAGUGA | Ms | [1686-1704] | [1479-1497] |
| 167 | AUGGCUCAGCAUGGGAUGA | UCAUCCCAUGCUGAGCCAU | | [998-1016] | [788-806] |
| 168 | AGCUUUGUCAAUAAGCCCA | UGGGCUUAUUGACAAAGCU | Rat, Ms, GP, Chn | [415-433] | [205-223] |
| 169 | GCUUUGAUGGCUCAGCAUG | CAUGCUGAGCCAUCAAAGC | | [992-1010] | [782-800] |
| 170 | AACCCACCCAAACUGGUCA | UGACCAGUUUGGGUGGGUU | | [952-970] | [742-760] |
| 171 | UGUCAGUCACCUUAUCAGC | GCUGAUAAGGUGACUGACA | | [1653-1671] | [1446-1464] |
| 172 | GUGUCUACAGCAACCUGUG | CACAGGUUGCUGUAGACAC | Rat | [1719-1737] | [1512-1530] |
| 173 | CUUGCCCAGGAUGUCAUCA | UGAUGACAUCCUGGGCAAG | GP, Chn | [886-904] | [676-694] |
| 174 | AAUCUAUCUCAUUUGCAUC | GAUGCAAAUGAGAUAGAUU | | [773-791] | [563-581] |
| 175 | GGGAGCUUUGUCAAUAAGC | GCUUAUUGACAAAGCUCCC | Rat, Ms, GP, Chn | [412-430] | [202-220] |
| 176 | CAUGGACAAUCACUUGCCC | GGGCAAGUGAUUGUCCAUG | Ms | [1677-1695] | [1470-1488] |
| 177 | GAUCCCUCCUAUGUCAACG | CGUUGACAUAGGAGGGAUC | GP | [1270-1288] | [1063-1081] |
| 178 | UGGGAGCUACAUUGCCUGU | ACAGGCAAUGUAGCUCCCA | | [1169-1187] | [959-977] |
| 179 | GAGCUACAUUGCCUGUAGG | CCUACAGGCAAUGUAGCUC | | [1172-1190] | [962-980] |
| 180 | UGGUUCGGUACAUGGGUUG | CAACCCAUGUACCGAACCA | Rat | [488-506] | [278-296] |
| 181 | ACUAAGGAUCACCGCUUUG | CAAAGCGGUGAUCCUUAGU | | [1630-1648] | [1423-1441] |
| 182 | UUGAUGAUCCCUCCUAUGU | ACAUAGGAGGGAUCAUCAA | | [1265-1283] | [1058-1076] |
| 183 | CCUGUGAAUCAGAGAGCCU | AGGCUCUCUGAUUCACAGG | Rat, Ms, GP, Chn | [841-859] | [631-649] |
| 184 | GUAACCUGAAAUUUGCUGG | CCAGCAAAUUUCAGGUUAC | | [668-686] | [458-476] |
| 185 | UUGAUGGCUCAGCAUGGGA | UCCCAUGCUGAGCCAUCAA | | [995-1013] | [785-803] |
| 186 | AUGAACAAGCUGAGUGGAG | CUCCACUCAGCUUGUUCAU | Rat, Ms, GP, Chn | [331-349] | [121-139] |
| 187 | UACCACAUGGACAAUCACU | AGUGAUUGUCCAUGUGGUA | Rat, Ms | [1672-1690] | [1465-1483] |
| 188 | GACUGCAAACAGAUCAUCG | CGAUGAUCUGUUUGCAGUC | | [739-757] | [529-547] |
| 189 | CAUCAGUCUGGUGUGUGAG | CUCACACACCAGACUGAUG | GP, Chn | [573-591] | [363-381] |
| 190 | GUUCGGUACAUGGGUUGUG | CACAACCCAUGUACCGAAC | | [490-508] | [280-298] |
| 191 | GUUUGACAUGAAGCCCUUC | GAAGGGCUUCAUGUCAAAC | | [1374-1392] | [1167-1185] |
| 192 | AAUCCUGCUAUCAAUGGCA | UGCCAUUGAUAGCAGGAUU | | [1339-1357] | [1132-1150] |
| 193 | AAGGAGGAGAAAGCCCUGU | ACAGGGCUUUCUCCUCCUU | | [618-636] | [408-426] |
| 194 | ACCGCUUUGAAAGUGUCAG | CUGACACUUUCAAAGCGGU | | [1640-1658] | [1433-1451] |
| 195 | AUGUGCUCACUGGCUUGCA | UGCAAGCCAGUGAGCACAU | GP | [1559-1577] | [1352-1370] |
| 196 | CCAAUCCUGCUAUCAAUGG | CCAUUGAUAGCAGGAUUGG | | [1337-1355] | [1130-1148] |
| 197 | UUUGCUGGAAUGCCAAUCA | UGAUUGGCAUUCCAGCAAA | Ms | [679-697] | [469-487] |
| 198 | GUCAACGUCCAGAACCUAG | CUAGGUUCUGGACGUUGAC | GP | [1282-1300] | [1075-1093] |
| 199 | AUGUCAUCAGCACCAUUGG | CCAAUGGUGCUGAUGACAU | GP, Chn | [896-914] | [686-704] |
| 200 | AAGACCCUGUGAAUCAGAG | CUCUGAUUCACAGGGUCUU | Rat, Ms, GP, Chn | [836-854] | [626-644] |
| 201 | CCUAUGUUGCCAAAGACCC | GGGUCUUUGGCAACAUAGG | Rat, Ms, GP, Chn | [824-842] | [614-632] |
| 202 | UAUGUCGCCUAUGUUGCCA | UGGCAACAUAGGCGACAUA | Chn | [817-835] | [607-625] |
| 203 | AGGAUCACCGCUUUGAAAG | CUUUCAAAGCGGUGAUCCU | | [1634-1652] | [1427-1445] |
| 204 | CUGUGUCUACAGCAACCUG | CAGGUUGCUGUAGACACAG | Rat | [1717-1735] | [1510-1528] |
| 205 | GAGCUUUGUCAAUAAGCCC | GGGCUUAUUGACAAAGCUC | Rat, Ms, GP, Chn | [414-432] | [204-222] |
| 206 | GAAGCCCUUCGAAGAUGCU | AGCAUCUUCGAAGGGCUUC | | [1383-1401] | [1176-1194] |
| 207 | UAUCAGCUACCACAUGGAC | GUCCAUGUGGUAGCUGAUA | | [1665-1683] | [1458-1476] |
| 208 | CAACCACCACAUGCAAUCU | AGAUUGCAUGUGGUGGUUG | | [759-777] | [549-567] |
| 209 | UCCUGCUAUCAAUGGCAGU | ACUGCCAUUGAUAGCAGGA | | [1341-1359] | [1134-1152] |
| 210 | UCACCUUAUCAGCUACCAC | GUGGUAGCUGAUAAGGUGA | | [1659-1677] | [1452-1470] |
| 211 | AUCCCUCCUAUGUCAACGU | ACGUUGACAUAGGAGGGAU | GP | [1271-1289] | [1064-1082] |
| 212 | AGUAACCUGAAAUUUGCUG | CAGCAAAUUUCAGGUUACU | | [667-685] | [457-475] |
| 213 | UGUGUCUACAGCAACCUGU | ACAGGUUGCUGUAGACACA | Rat | [1718-1736] | [1511-1529] |
| 214 | AUCACUUGCCCAUCAUCUC | GAGAUGAUGGGCAAGUGAU | Ms | [1685-1703] | [1478-1496] |
| 215 | GUCUACAGCAACCUGUGGA | UCCACAGGUUGCUGUAGAC | Rat | [1721-1739] | [1514-1532] |
| 216 | AGUAUGUCGCCUAUGUUGC | GCAACAUAGGCGACAUACU | Chn | [815-833] | [605-623] |
| 217 | AUGGACAAUCACUUGCCCA | UGGGCAAGUGAUUGUCCAU | Ms | [1678-1696] | [1471-1489] |
| 218 | CACUUGCCCAUCAUCUCUG | CAGAGAUGAUGGGCAAGUG | Ms | [1687-1705] | [1480-1498] |
| 219 | UACAUUGCCUGUAGGACAG | CUGUCCUACAGGCAAUGUA | | [1176-1194] | [966-984] |
| 220 | GACCUGUUUGACAUGAAGC | GCUUCAUGUCAAACAGGUC | | [1369-1387] | [1162-1180] |
| 221 | ACAUUCUGGAGUGUCCCGA | UCGGGACACUCCAGAAUGU | | [863-881] | [653-671] |
| 222 | GGUUUCCUACUUGGUUCGG | CCGAACCAAGUAGGAAACC | Rat, Ms, GP, Chn | [477-495] | [267-285] |
| 223 | UUGCUGGAAUGCCAAUCAC | GUGAUUGGCAUUCCAGCAA | Ms | [680-698] | [470-488] |
| 224 | UGUGAAUCAGAGAGCCUGC | GCAGGCUCUCUGAUUCACA | Rat, Ms, Chn | [843-861] | [633-651] |
| 225 | AUGGCUGGCUUUGAUGGCU | AGCCAUCAAAGCCAGCCAU | Rat, Ms | [985-1003] | [775-793] |
| 226 | CAAUCCUGCUAUCAAUGGC | GCCAUUGAUAGCAGGAUUG | | [1338-1356] | [1131-1149] |
| 227 | AACCUGUGGAGCGGAAACU | AGUUUCCGCUCCACAGGUU | | [1730-1748] | [1523-1541] |
| 228 | AACUGUGUCUACAGCAACC | GGUUGCUGUAGACACAGUU | | [1715-1733] | [1508-1526] |
| 229 | GCUACAUUGCCUGUAGGAC | GUCCUACAGGCAAUGUAGC | | [1174-1192] | [964-982] |
| 230 | UCCCAACGACAAAGUCAUG | CAUGACUUUGUCGUUGGGA | Rat, Ms, GP, Chn | [450-468] | [240-258] |
| 231 | GUCGCCUAUGUUGCCAAAG | CUUUGGCAACAUAGGCGAC | Chn | [820-838] | [610-628] |
| 232 | UCUACAGCAACCUGUGGAG | CUCCACAGGUUGCUGUAGA | | [1722-1740] | [1515-1533] |
| 233 | AAACAGAUGCCACCUCCAC | GUGGAGGUGGCAUCUGUUU | | [1222-1240] | [1012-1030] |
| 234 | CUGUGAAUCAGAGAGCCUG | CAGGCUCUCUGAUUCACAG | Rat, Ms, GP, Chn | [842-860] | [632-650] |
| 235 | AGUACUAUAAUGACUUCCC | GGGAAGUCAUUAUAGUACU | GP, Chn | [1043-1061] | [833-851] |
| 236 | AGCGAACUGUGUCUACAGC | GCUGUAGACACAGUUCGCU | | [1711-1729] | [1504-1522] |
| 237 | AUCACUCUCACCGUCUCCA | UGGAGACGGUGAGAGUGAU | | [694-712] | [484-502] |
| 238 | ACGACAAAGUCAUGGGACC | GGUCCCAUGACUUUGUCGU | Rat, Ms, GP, Chn | [455-473] | [245-263] |
| 239 | UUGAAAGUGUCAGUCACCU | AGGUGACUGACACUUUCAA | | [1646-1664] | [1439-1457] |
| 240 | UACUAUAAUGACUUCCCGG | CCGGGAAGUCAUUAUAGUA | | [1045-1063] | [835-853] |
| 241 | UGUCUACAGCAACCUGUGG | CCACAGGUUGCUGUAGACA | Rat | [1720-1738] | [1513-1531] |
| 242 | AACGUCCAGAACCUAGACA | UGUCUAGGUUCUGGACGUU | GP | [1285-1303] | [1078-1096] |
| 243 | AACAAUACCUCAGGAACCC | GGGUUCCUGAGGUAUUGUU | GP | [938-956] | [728-746] |
| 244 | AAUACCUCAGGAACCCACC | GGUGGGUUCCUGAGGUAUU | GP | [941-959] | [731-749] |
| 245 | UCCAGAACCUAGACAAGGC | GCCUUGUCUAGGUUCUGGA | GP, Chn | [1289-1307] | [1082-1100] |
| 246 | AUUCUGGAGUGUCCCGAAG | CUUCGGGACACUCCAGAAU | | [865-883] | [655-673] |
| 247 | CUAUCAAUGGCAGUGCACC | GGUGCACUGCCAUUGAUAG | | [1346-1364] | [1139-1157] |
| 248 | UUGGGAGCUACAUUGCCUG | CAGGCAAUGUAGCUCCCAA | | [1168-1186] | [958-976] |
| 249 | UUCCUACUUGGUUCGGUAC | GUACCGAACCAAGUAGGAA | Rat, Ms, GP, Chn | [480-498] | [270-288] |
| 250 | UAAGCAUUUGCUACUGGUG | CACCAGUAGCAAAUGCUUA | | [1590-1608] | [1383-1401] |
| 251 | ACUGCAAACAGAUCAUCGC | GCGAUGAUCUGUUUGCAGU | | [740-758] | [530-548] |
| 252 | AGCCCUUCGAAGAUGCUCU | AGAGCAUCUUCGAAGGGCU | | [1385-1403] | [1178-1196] |
| 253 | UUCGAGUUGCGCUUCAAAC | GUUUGAAGCGCAACUCGAA | | [922-940] | [712-730] |
| 254 | CUGCAAACAGAUCAUCGCC | GGCGAUGAUCUGUUUGCAG | | [741-759] | [531-549] |
| 255 | UGUUGCCAAAGACCCUGUG | CACAGGGUCUUUGGCAACA | Rat, Ms, GP, Chn | [828-846] | [618-636] |
| 256 | CAGUACUAUAAUGACUUCC | GGAAGUCAUUAUAGUACUG | GP, Chn | [1042-1060] | [832-850] |
| 257 | AUGUUGCCAAAGACCCUGU | ACAGGGUCUUUGGCAACAU | Rat, Ms, GP, Chn | [827-845] | [617-635] |
| 258 | UGGAAUGCCAAUCACUCUC | GAGAGUGAUUGGCAUUCCA | Ms, Chn | [684-702] | [474-492] |
| 259 | AUCAUCGCCAACCACCACA | UGUGGUGGUUGGCGAUGAU | | [751-769] | [541-559] |
| 260 | UUGCCCAGGAUGUCAUCAG | CUGAUGACAUCCUGGGCAA | GP, Chn | [887-905] | [677-695] |
| 261 | UUGCGCUUCAAACAAUACC | GGUAUUGUUUGAAGCGCAA | | [928-946] | [718-736] |
| 262 | AUCACCGCUUUGAAAGUGU | ACACUUUCAAAGCGGUGAU | | [1637-1655] | [1430-1448] |
| 263 | UACAUGGGUUGUGUGGAGG | CCUCCACACAACCCAUGUA | | [496-514] | [286-304] |
| 264 | UUUGAUGGCUCAGCAUGGG | CCCAUGCUGAGCCAUCAAA | | [994-1012] | [784-802] |
| 265 | CUGAAAUUUGCUGGAAUGC | GCAUUCCAGCAAAUUUCAG | | [673-691] | [463-481] |
| 266 | CCUUCGAAGAUGCUCUUCG | CGAAGAGCAUCUUCGAAGG | | [1388-1406] | [1181-1199] |
| 267 | CUUAUCAGCUACCACAUGG | CCAUGUGGUAGCUGAUAAG | | [1663-1681] | [1456-1474] |
| 268 | UAUGUUGCCAAAGACCCUG | CAGGGUCUUUGGCAACAUA | Rat, Ms, GP, Chn | [826-844] | [616-634] |
| 269 | CACUUGGGAGCUACAUUGC | GCAAUGUAGCUCCCAAGUG | | [1165-1183] | [955-973] |
| 270 | GUCCAGAACCUAGACAAGG | CCUUGUCUAGGUUCUGGAC | GP, Chn | [1288-1306] | [1081-1099] |
| 271 | AUCUCAUUUGCAUCCGGCG | CGCCGGAUGCAAAUGAGAU | | [778-796] | [568-586] |
| 272 | ACUAUAAUGACUUCCCGGG | CCCGGGAAGUCAUUAUAGU | | [1046-1064] | [836-854] |
| 273 | UAUCAAUGGCAGUGCACCC | GGGUGCACUGCCAUUGAUA | | [1347-1365] | [1140-1158] |
| 274 | GAUGUCAUCAGCACCAUUG | CAAUGGUGCUGAUGACAUC | GP, Chn | [895-913] | [685-703] |
| 275 | UGAAGCCCUUCGAAGAUGC | GCAUCUUCGAAGGGCUUCA | Rat | [1382-1400] | [1175-1193] |
| 276 | ACAUGGACAAUCACUUGCC | GGCAAGUGAUUGUCCAUGU | Ms | [1676-1694] | [1469-1487] |
| 277 | ACUUGCCCAUCAUCUCUGC | GCAGAGAUGAUGGGCAAGU | Ms | [1688-1706] | [1481-1499] |
| 278 | AAUGCCAAUCACUCUCACC | GGUGAGAGUGAUUGGCAUU | | [687-705] | [477-495] |
| 279 | ACCUGUUUGACAUGAAGCC | GGCUUCAUGUCAAACAGGU | | [1370-1388] | [1163-1181] |
| 280 | AUCAGUCUGGUGUGUGAGG | CCUCACACACCAGACUGAU | GP, Chn | [574-592] | [364-382] |
| 281 | CCUGUUUGACAUGAAGCCC | GGGCUUCAUGUCAAACAGG | | [1371-1389] | [1164-1182] |
| 282 | UAUGUCAACGUCCAGAACC | GGUUCUGGACGUUGACAUA | GP | [1279-1297] | [1072-1090] |
| 283 | UUUGAAAGUGUCAGUCACC | GGUGACUGACACUUUCAAA | | [1645-1663] | [1438-1456] |
| 284 | UGAAAUUUGCUGGAAUGCC | GGCAUUCCAGCAAAUUUCA | | [674-692] | [464-482] |
| 285 | CACAUGGACAAUCACUUGC | GCAAGUGAUUGUCCAUGUG | Ms | [1675-1693] | [1468-1486] |
| 286 | AUUUGCUACUGGUGGACCC | GGGUCCACCAGUAGCAAAU | | [1595-1613] | [1388-1406] |
| 287 | AAGCAUUUGCUACUGGUGG | CCACCAGUAGCAAAUGCUU | | [1591-1609] | [1384-1402] |
| 288 | GUUCGGACUAAGGAUCACC | GGUGAUCCUUAGUCCGAAC | | [1624-1642] | [1417-1435] |
| 289 | UAUCUCAUUUGCAUCCGGC | GCCGGAUGCAAAUGAGAUA | | [777-795] | [567-585] |
| 290 | AACGACAAAGUCAUGGGAC | GUCCCAUGACUUUGUCGUU | Rat, Ms, GP, Chn | [454-472] | [244-262] |
| 291 | AUUGCCUGUAGGACAGCCU | AGGCUGUCCUACAGGCAAU | | [1179-1197] | [969-987] |
| 292 | ACUGUGUCUACAGCAACCU | AGGUUGCUGUAGACACAGU | | [1716-1734] | [1509-1527] |
| 293 | UUGUCAAUAAGCCCACGCG | CGCGUGGGCUUAUUGACAA | | [419-437] | [209-227] |
| 294 | GAAGUCCGCAAACAGAUGC | GCAUCUGUUUGCGGACUUC | | [1213-1231] | [1003-1021] |
| 295 | UCUAUCUCAUUUGCAUCCG | CGGAUGCAAAUGAGAUAGA | | [775-793] | [565-583] |
| 296 | ACUUGGGAGCUACAUUGCC | GGCAAUGUAGCUCCCAAGU | | [1166-1184] | [956-974] |
| 297 | AUCCUGCUAUCAAUGGCAG | CUGCCAUUGAUAGCAGGAU | | [1340-1358] | [1133-1151] |
| 298 | AUGAAGCCCUUCGAAGAUG | CAUCUUCGAAGGGCUUCAU | Rat | [1381-1399] | [1174-1192] |
| 299 | UUUGCUACUGGUGGACCCU | AGGGUCCACCAGUAGCAAA | | [1596-1614] | [1389-1407] |
| 300 | GAUCACCGCUUUGAAAGUG | CACUUUCAAAGCGGUGAUC | | [1636-1654] | [1429-1447] |
| 301 | AUGCCAAUCACUCUCACCG | CGGUGAGAGUGAUUGGCAU | | [688-706] | [478-496] |
| 302 | UUCGGACUAAGGAUCACCG | CGGUGAUCCUUAGUCCGAA | | [1625-1643] | [1418-1436] |
| 303 | UGUCAUCAGCACCAUUGGC | GCCAAUGGUGCUGAUGACA | GP, Chn | [897-915] | [687-705] |
| 304 | AAUCACUCUCACCGUCUCC | GGAGACGGUGAGAGUGAUU | | [693-711] | [483-501] |
| 305 | ACCCUGUGAAUCAGAGAGC | GCUCUCUGAUUCACAGGGU | Rat, Ms, GP, Chn | [839-857] | [629-647] |
| 306 | CCCAACGACAAAGUCAUGG | CCAUGACUUUGUCGUUGGG | Rat, Ms, GP, Chn | [451-469] | [241-259] |
| 307 | AUCUAUCUCAUUUGCAUCC | GGAUGCAAAUGAGAUAGAU | | [774-792] | [564-582] |
| 308 | CAUUUGCUACUGGUGGACC | GGUCCACCAGUAGCAAAUG | | [1594-1612] | [1387-1405] |
| 309 | AAGUCCGCAAACAGAUGCC | GGCAUCUGUUUGCGGACUU | | [1214-1232] | [1004-1022] |
| 310 | AGUCACCUUAUCAGCUACC | GGUAGCUGAUAAGGUGACU | | [1657-1675] | [1450-1468] |
| 311 | ACAUUGCCUGUAGGACAGC | GCUGUCCUACAGGCAAUGU | | [1177-1195] | [967-985] |
| 312 | UUUGACAUGAAGCCCUUCG | CGAAGGGCUUCAUGUCAAA | Rat | [1375-1393] | [1168-1186] |
| 313 | UGCCACAUUCUGGAGUGUC | GACACUCCAGAAUGUGGCA | | [859-877] | [649-667] |
| 314 | CUGCUAUCAAUGGCAGUGC | GCACUGCCAUUGAUAGCAG | | [1343-1361] | [1136-1154] |
| 315 | AUUUGCUGGAAUGCCAAUC | GAUUGGCAUUCCAGCAAAU | | [678-696] | [468-486] |
| 316 | AAACAGAUCAUCGCCAACC | GGUUGGCGAUGAUCUGUUU | | [745-763] | [535-553] |
| 317 | AAGCCCUUCGAAGAUGCUC | GAGCAUCUUCGAAGGGCUU | | [1384-1402] | [1177-1195] |
| 318 | UAUGUGCUCACUGGCUUGC | GCAAGCCAGUGAGCACAUA | GP | [1558-1576] | [1351-1369] |
| 319 | UUUGUCAAUAAGCCCACGC | GCGUGGGCUUAUUGACAAA | | [418-436] | [208-226] |

TABLE B9
ZNHIT1 19-mer oligomer pairs zinc finger, HIT type 1

| | | | | |
|---|---|---|---|---|
| No. | Sense | AntiSense | Other Sp | Human-37594439: 493-957 |

| | | | | |
|---|---|---|---|---|
| 1 | CCGAGGUGAUCAUUUUAAA | UUUAAAAUGAUCACCUCGG | | [705-723] |
| 2 | CCCGAGGUGAUCAUUUUAA | UUAAAAUGAUCACCUCGGG | | [704-722] |
| 3 | CGUGACCACAUCUUUAAAA | UUUUAAAGAUGUGGUCACG | | [240-258] |
| 4 | CCCUAUAAAACAUGGCGAA | UUCGCCAUGUUUUAUAGGG | | [321-339] |
| 5 | GUGACCACAUCUUUAAAAU | AUUUUAAAGAUGUGGUCAC | | [241-259] |
| 6 | GAAAAAACUUUCAGGCCCU | AGGGCCUGAAAGUUUUUUC | | [734-752] |
| 7 | CCCUGGAGAAUGACAACUU | AAGUUGUCAUUCUCCAGGG | | [593-611] |
| 8 | CUAUAAAACAUGGCGAAAA | UUUUCGCCAUGUUUUAUAG | | [323-341] |
| 9 | CCGUGACCACAUCUUUAAA | UUUAAAGAUGUGGUCACGG | | [239-257] |
| 10 | ACCGUGACCACAUCUUUAA | UUAAAGAUGUGGUCACGGU | | [238-256] |
| 11 | CAGAAAGACAGAAUUUCAU | AUGAAAUUCUGUCUUUCUG | | [1006-1024] |
| 12 | CGGACACUGGAAAGAAAAA | UUUUUCUUUCCAGUGUCCG | CHL | [677-695] |
| 13 | CAGUGCUGUUAGAAUAAAA | UUUUAUUCUAACAGCACUG | | [1156-1174] |
| 14 | GCUCAUUCACCCAACAAAA | UUUUGUUGGGUGAAUGAGC | | [1068-1086] |
| 15 | UCAUUUUAAACUUCGCUUC | GAAGCGAAGUUUAAAAUGA | | [714-732] |
| 16 | GAGGUGAUCAUUUUAAACU | AGUUUAAAAUGAUCACCUC | | [707-725] |
| 17 | CGAGGUGAUCAUUUUAAAC | GUUUAAAAUGAUCACCUCG | | [706-724] |
| 18 | GCGGACACUGGAAAGAAAA | UUUUCUUUCCAGUGUCCGC | CHL | [676-694] |
| 19 | CGCCACGUAAUGAGUCAAA | UUUGACUCAUUACGUGGCG | | [363-381] |
| 20 | CCUUCAGAAAGACAGAAUU | AAUUCUGUCUUUCUGAAGG | | [1002-1020] |
| 21 | CCUAUAAAACAUGGCGAAA | UUUCGCCAUGUUUUAUAGG | | [322-340] |
| 22 | ACAGCAUCUUCACAAGGAC | GUCCUUGUGAAGAUGCUGU | | [190-208] |
| 23 | CACAGCAUCUUCACAAGGA | UCCUUGUGAAGAUGCUGUG | | [189-207] |
| 24 | CCCAGUGCUGUUAGAAUAA | UUAUUCUAACAGCACUGGG | | [1154-1172] |
| 25 | ACCCGAGGUGAUCAUUUUA | UAAAAUGAUCACCUCGGGU | | [703-721] |
| 26 | AACCCGAGGUGAUCAUUUU | AAAAUGAUCACCUCGGGUU | | [702-720] |
| 27 | GCCCUGGAGAAUGACAACU | AGUUGUCAUUCUCCAGGGC | | [592-610] |
| 28 | ACACCGUGACCACAUCUUU | AAAGAUGUGGUCACGGUGU | | [236-254] |
| 29 | GCAUCUUCACAAGGACCAA | UUGGUCCUUGUGAAGAUGC | | [193-211] |
| 30 | GAUCACAGCAUCUUCACAA | UUGUGAAGAUGCUGUGAUC | | [186-204] |
| 31 | GGAGCAGAACUUGAGUGUG | CACACUCAAGUUCUGCUCC | | [759-777] |
| 32 | UGAUCAUUUUAAACUUCGC | GCGAAGUUUAAAAUGAUCA | | [711-729] |
| 33 | GUGAUCAUUUUAAACUUCG | CGAAGUUUAAAAUGAUCAC | | [710-728] |
| 34 | CUGGAAAGAAAAAGAAGAA | UUCUUCUUUUUCUUUCCAG | CHL | [683-701] |
| 35 | AGGCCCUGGAGAAUGACAA | UUGUCAUUCUCCAGGGCCU | | [590-608] |
| 36 | GUACAAGCUACCGGAAGUG | CACUUCCGGUAGCUUGUAC | | [402-420] |
| 37 | CACCGUGACCACAUCUUUA | UAAAGAUGUGGUCACGGUG | | [237-255] |
| 38 | GACCAAAGGAAAAUAAGAU | AUCUUAUUUUCCUUUGGUC | | [206-224] |
| 39 | CUGUUAGAAUAAAAAGCCU | AGGCUUUUUAUUCUAACAG | | [1161-1179] |
| 40 | UCAGAAAGACAGAAUUUCA | UGAAAUUCUGUCUUUCUGA | | [1005-1023] |
| 41 | AGGAGCAGAACUUGAGUGU | ACACUCAAGUUCUGCUCCU | | [758-776] |
| 42 | CCACUUUCUUUGACAGUCC | GGACUGUCAAAGAAAGUGG | | [120-138] |
| 43 | GGAGGAGCAGAACUUGAGU | ACUCAAGUUCUGCUCCUCC | | [756-774] |
| 44 | ACAAGCUACCGGAAGUGAU | AUCACUUCCGGUAGCUUGU | | [404-422] |
| 45 | AGAACACCGUGACCACAUC | GAUGUGGUCACGGUGUUCU | | [233-251] |
| 46 | CAAGGACCAAAGGAAAAUA | UAUUUUCCUUUGGUCCUUG | | [202-220] |
| 47 | AGCAUCUUCACAAGGACCA | UGGUCCUUGUGAAGAUGCU | | [192-210] |
| 48 | UGUCUUAUCUGCCAGGAAA | UUUCCUGGCAGAUAAGACA | | [1090-1108] |
| 49 | CUUCAGAAAGACAGAAUUU | AAAUUCUGUCUUUCUGAAG | | [1003-1021] |
| 50 | ACACUGGAAAGAAAAAGAA | UUCUUUUUCUUUCCAGUGU | CHL | [680-698] |
| 51 | GCCACGUAAUGAGUCAAAG | CUUUGACUCAUUACGUGGC | | [364-382] |
| 52 | GGAAACGCCACGUAAUGAG | CUCAUUACGUGGCGUUUCC | | [358-376] |
| 53 | CAAGGAAACGCCACGUAAU | AUUACGUGGCGUUUCCUUG | | [355-373] |
| 54 | GGACCAAAGGAAAAUAAGA | UCUUAUUUUCCUUUGGUCC | | [205-223] |
| 55 | ACAAGGACCAAAGGAAAAU | AUUUUCCUUUGGUCCUUGU | | [201-219] |
| 56 | GUGUCUUAUCUGCCAGGAA | UUCCUGGCAGAUAAGACAC | | [1089-1107] |
| 57 | CACUUUCUUUGACAGUCCA | UGGACUGUCAAAGAAAGUG | | [121-139] |
| 58 | ACGAUGCGGACACUGGAAA | UUUCCAGUGUCCGCAUCGU | | [671-689] |
| 59 | AUGGUGGAGAAGAAAACUU | AAGUUUUCUUCUCCACCAU | | [493-511] |
| 60 | GCAGUUUCUUCCGACAGUU | AACUGUCGGAAGAAACUGC | | [463-481] |
| 61 | CAUCUUCACAAGGACCAAA | UUUGGUCCUUGUGAAGAUG | | [194-212] |
| 62 | UGUUGGAGGAGCAGAACUU | AAGUUCUGCUCCUCCAACA | | [752-770] |
| 63 | GGACACUGGAAAGAAAAAG | CUUUUUCUUUCCAGUGUCC | CHL | [678-696] |
| 64 | ACGCCACGUAAUGAGUCAA | UUGACUCAUUACGUGGCGU | | [362-380] |
| 65 | AAGGACCAAAGGAAAAUAA | UUAUUUUCCUUUGGUCCUU | | [203-221] |
| 66 | UCAGUUUGAUGACGAUGCG | CGCAUCGUCAUCAAACUGA | | [660-678] |
| 67 | CGUAAUGAGUCAAAGCUGU | ACAGCUUUGACUCAUUACG | | [368-386] |
| 68 | CACGUAAUGAGUCAAAGCU | AGCUUUGACUCAUUACGUG | | [366-384] |
| 69 | UCACAAGGACCAAAGGAAA | UUUCCUUUGGUCCUUGUGA | | [199-217] |
| 70 | GCCUUUGUGAUCACAGCAU | AUGCUGUGAUCACAAAGGC | | [178-196] |
| 71 | GCAAGAGACUGCCUCAGUU | AACUGAGGCAGUCUCUUGC | | [647-665] |
| 72 | CACAAGGACCAAAGGAAAA | UUUUCCUUUGGUCCUUGUG | | [200-218] |
| 73 | CCACGUAAUGAGUCAAAGC | GCUUUGACUCAUUACGUGG | | [365-383] |
| 74 | ACAGAAUUUCAUCACCCAA | UUGGGUGAUGAAAUUCUGU | | [1013-1031] |
| 75 | GACACUGGAAAGAAAAAGA | UCUUUUUCUUUCCAGUGUC | CHL | [679-697] |
| 76 | GGAGAAGAAAACUUCGGUU | AACCGAAGUUUUCUUCUCC | | [498-516] |
| 77 | CCAACAAAACUGUGUCUUA | UAAGACACAGUUUUGUUGG | | [1078-1096] |
| 78 | UCGCUUCCGAAAAAACUUU | AAAGUUUUUUCGGAAGCGA | | [726-744] |
| 79 | AAACUUCGCUUCCGAAAAA | UUUUUCGGAAGCGAAGUUU | GP | [721-739] |
| 80 | CGCGCAGAAGUACAAGCUA | UAGCUUGUACUUCUGCGCG | | [393-411] |
| 81 | ACAUCUUUAAAAUGACCCA | UGGGUCAUUUUAAAGAUGU | | [247-265] |
| 82 | ACCACAUCUUUAAAAUGAC | GUCAUUUUAAAGAUGUGGU | | [244-262] |
| 83 | CCCAACAAAACUGUGUCUU | AAGACACAGUUUUGUUGGG | | [1077-1095] |
| 84 | CGCUCAUUCACCCAACAAA | UUUGUUGGGUGAAUGAGCG | | [1067-1085] |
| 85 | AAAACUUUCAGGCCCUGUU | AACAGGGCCUGAAAGUUUU | | [737-755] |
| 86 | CGAAAAAACUUUCAGGCCC | GGGCCUGAAAGUUUUUUCG | | [733-751] |
| 87 | GAAGUACAAGCUACCGGAA | UUCCGGUAGCUUGUACUUC | | [399-417] |
| 88 | AACUUCGCUUCCGAAAAAA | UUUUUUCGGAAGCGAAGUU | GP | [722-740] |
| 89 | AGAAGUACAAGCUACCGGA | UCCGGUAGCUUGUACUUCU | | [398-416] |
| 90 | CCACUAGGAACCUCGGAUU | AAUCCGAGGUUCCUAGUGG | | [86-104] |
| 91 | ACAAGAUUUACACCUCCAC | GUGGAGGUGUAAAUCUUGU | | [279-297] |
| 92 | CACAAGAUUUACACCUCCA | UGGAGGUGUAAAUCUUGUG | | [278-296] |
| 93 | GGCUCCCACAAGAUUUACA | UGUAAAUCUUGUGGGAGCC | | [272-290] |
| 94 | CCACAUCUUUAAAAUGACC | GGUCAUUUUAAAGAUGUGG | | [245-263] |
| 95 | CGCUUCCGAAAAAACUUUC | GAAAGUUUUUUCGGAAGCG | | [727-745] |
| 96 | GAGACUGCCUCAGUUUGAU | AUCAAACUGAGGCAGUCUC | | [651-669] |
| 97 | CAAGAGACUGCCUCAGUUU | AAACUGAGGCAGUCUCUUG | | [648-666] |
| 98 | UCCAAGGAAACGCCACGUA | UACGUGGCGUUUCCUUGGA | | [353-371] |
| 99 | GACCACAUCUUUAAAAUGA | UCAUUUUAAAGAUGUGGUC | | [243-261] |
| 100 | CCGCUCAUUCACCCAACAA | UUGUUGGGUGAAUGAGCGG | | [1066-1084] |
| 101 | GACAGAAUUUCAUCACCCA | UGGGUGAUGAAAUUCUGUC | | [1012-1030] |
| 102 | GCCUUCAGAAAGACAGAAU | AUUCUGUCUUUCUGAAGGC | | [1001-1019] |
| 103 | GAAAACCCGAGGUGAUCAU | AUGAUCACCUCGGGUUUUC | | [699-717] |
| 104 | GGAGAAUGACAACUUCCAG | CUGGAAGUUGUCAUUCUCC | CHL, Ms | [597-615] |
| 105 | CAAUGGUGGAGAAGAAAAC | GUUUUCUUCUCCACCAUUG | | [491-509] |
| 106 | GCGCAGAAGUACAAGCUAC | GUAGCUUGUACUUCUGCGC | | [394-412] |
| 107 | CAACAAAACUGUGUCUUAU | AUAAGACACAGUUUUGUUG | | [1079-1097] |
| 108 | GCUUCCGAAAAAACUUUCA | UGAAAGUUUUUUCGGAAGC | | [728-746] |
| 109 | CCCACAAGAUUUACACCUC | GAGGUGUAAAUCUUGUGGG | | [276-294] |
| 110 | GCUCCCACAAGAUUUACAC | GUGUAAAUCUUGUGGGAGC | | [273-291] |
| 111 | GAAAGACAGAAUUUCAUCA | UGAUGAAAUUCUGUCUUUC | | [1008-1026] |
| 112 | GCCUCAGUUUGAUGACGAU | AUCGUCAUCAAACUGAGGC | | [657-675] |
| 113 | GAGAAGAAAACUUCGGUUC | GAACCGAAGUUUUCUUCUC | | [499-517] |
| 114 | CAGUUGUGUUGUGCCAAUG | CAUUGGCACAACACAACUG | | [477-495] |
| 115 | CGACAGUUGUGUUGUGCCA | UGGCACAACACAACUGUCG | | [474-492] |
| 116 | GCAGCAGUUUCUUCCGACA | UGUCGGAAGAAACUGCUGC | | [460-478] |
| 117 | GCUUUCUUGUCUCCAAGGA | UCCUUGGAGACAAGAAAGC | | [342-360] |
| 118 | CCACAAGAUUUACACCUCC | GGAGGUGUAAAUCUUGUGG | | [277-295] |
| 119 | CAUCUUUAAAAUGACCCAU | AUGGGUCAUUUUAAAGAUG | | [248-266] |
| 120 | UGACCACAUCUUUAAAAUG | CAUUUUAAAGAUGUGGUCA | | [242-260] |
| 121 | CCUUUGUGAUCACAGCAUC | GAUGCUGUGAUCACAAAGG | | [179-197] |
| 122 | AGACAGAAUUUCAUCACCC | GGGUGAUGAAAUUCUGUCU | | [1011-1029] |
| 123 | GGCCUUCAGAAAGACAGAA | UUCUGUCUUUCUGAAGGCC | | [1000-1018] |
| 124 | UGAUGACGAUGCGGACACU | AGUGUCCGCAUCGUCAUCA | | [666-684] |
| 125 | UGGAGAAGAAAACUUCGGU | ACCGAAGUUUUCUUCUCCA | | [497-515] |
| 126 | UUGUGCCAAUGGUGGAGAA | UUCUCCACCAUUGGCACAA | | [485-503] |
| 127 | CCAAGGAAACGCCACGUAA | UUACGUGGCGUUUCCUUGG | | [354-372] |
| 128 | UGAGGCCGGAAGUGGUUUU | AAAACCACUUCCGGCCUCA | | [300-318] |
| 129 | ACAAAACUGUGUCUUAUCU | AGAUAAGACACAGUUUUGU | | [1081-1099] |
| 130 | CGCAGAAGUACAAGCUACC | GGUAGCUUGUACUUCUGCG | | [395-413] |
| 131 | AGAUUUCUUGUAAGAACAC | GUGUUCUUACAAGAAAUCU | | [221-239] |
| 132 | UCACCCAACAAAACUGUGU | ACACAGUUUUGUUGGGUGA | | [1074-1092] |
| 133 | CGGCCUUCAGAAAGACAGA | UCUGUCUUUCUGAAGGCCG | | [999-1017] |
| 134 | GCCACUUUCUUUGACAGUC | GACUGUCAAAGAAAGUGGC | | [119-137] |
| 135 | AGAAUGACAACUUCCAGGA | UCCUGGAAGUUGUCAUUCU | CHL, Ms | [599-617] |
| 136 | AGCAGUUUCUUCCGACAGU | ACUGUCGGAAGAAACUGCU | | [462-480] |
| 137 | ACGCGCAGAAGUACAAGCU | AGCUUGUACUUCUGCGCGU | | [392-410] |
| 138 | GGAAGCCAGCCACUUUCUU | AAGAAAGUGGCUGGCUUCC | | [111-129] |
| 139 | GUGGCUCCCACAAGAUUUA | UAAAUCUUGUGGGAGCCAC | | [270-288] |
| 140 | GCUGUUAGAAUAAAAAGCC | GGCUUUUUAUUCUAACAGC | | [1160-1178] |
| 141 | ACCCAACAAAACUGUGUCU | AGACACAGUUUUGUUGGGU | | [1076-1094] |
| 142 | GGUGUCUGAAGUGGACUGU | ACAGUCCACUUCAGACACC | | [935-953] |
| 143 | GGCUGCCUUUGUGAUCACA | UGUGAUCACAAAGGCAGCC | | [174-192] |
| 144 | UGGAGAAUGACAACUUCCA | UGGAAGUUGUCAUUCUCCA | CHL, Ms | [596-614] |
| 145 | CAGCCACUUUCUUUGACAG | CUGUCAAAGAAAGUGGCUG | | [117-135] |
| 146 | CCAGCCACUUUCUUUGACA | UGUCAAAGAAAGUGGCUGG | | [116-134] |
| 147 | UCCCACAAGAUUUACACCU | AGGUGUAAAUCUUGUGGGA | | [275-293] |
| 148 | GGAGUUCAGCUUUCUGCAG | CUGCAGAAAGCUGAACUCC | | [63-81] |
| 149 | CACCCAACAAAACUGUGUC | GACACAGUUUUGUUGGGUG | | [1075-1093] |
| 150 | UGGUGGAGAAGAAAACUUC | GAAGUUUUCUUCUCCACCA | | [494-512] |
| 151 | GCCAAUGGUGGAGAAGAAA | UUUCUUCUCCACCAUUGGC | | [489-507] |
| 152 | GGAACUGGGAGUUCAGCUU | AAGCUGAACUCCCAGUUCC | | [56-74] |
| 153 | GAAGAAAACUUCGGUUCGC | GCGAACCGAAGUUUUCUUC | | [501-519] |
| 154 | GGGAGUUCAGCUUUCUGCA | UGCAGAAAGCUGAACUCCC | | [62-80] |
| 155 | UGCCUCAGUUUGAUGACGA | UCGUCAUCAAACUGAGGCA | | [656-674] |
| 156 | GCCAGCCACUUUCUUUGAC | GUCAAAGAAAGUGGCUGGC | | [115-133] |
| 157 | GAGACCAGGUGUCUGAAGU | ACUUCAGACACCUGGUCUC | Ms | [928-946] |
| 158 | CGUGGCUCCCACAAGAUUU | AAAUCUUGUGGGAGCCACG | | [269-287] |
| 159 | GAGUUCAGCUUUCUGCAGA | UCUGCAGAAAGCUGAACUC | | [64-82] |
| 160 | GAACUGGGAGUUCAGCUUU | AAAGCUGAACUCCCAGUUC | | [57-75] |
| 161 | GAAGCCAGCCACUUUCUUU | AAAGAAAGUGGCUGGCUUC | | [112-130] |
| 162 | CCAAUGGUGGAGAAGAAAA | UUUUCUUCUCCACCAUUGG | | [490-508] |

TABLE B10
ZNHIT1-19-mer oligomer pairs zinc finger, HIT type 1

| | | | | Human- |
|---|---|---|---|---|
| No. | Sense | AntiSense | Other Sp | 37594439: 493-957 |

| | | | | |
|---|---|---|---|---|
| 1 | GGAAAGAAAAAGAAGAAAA | UUUUCUUCUUUUUCUUUCC | Rat, Ms, GP, Chn | [685-703] |
| 2 | UGCGGACACUGGAAAGAAA | UUUCUUUCCAGUGUCCGCA | Chn | [675-693] |
| 3 | GAAAAAGAAGAAAACCCGA | UCGGGUUUUCUUCUUUUUC | | [690-708] |
| 4 | UGCCAAUGGUGGAGAAGAA | UUCUUCUCCACCAUUGGCA | | [488-506] |
| 5 | AGUGCUGUUAGAAUAAAAA | UUUUUAUUCUAACAGCACU | | [1157-1175] |
| 6 | GAAGAAAACCCGAGGUGAU | AUCACCUCGGGUUUUCUUC | | [696-714] |
| 7 | UGGAAAGAAAAAGAAGAAA | UUUCUUCUUUUUCUUUCCA | Chn | [684-702] |
| 8 | AGGAGACCAGGUGUCUGAA | UUCAGACACCUGGUCUCCU | Ms | [926-944] |
| 9 | GAAAGAAAAAGAAGAAAAC | GUUUUCUUCUUUUUCUUUC | Rat, Ms, GP, Chn | [686-704] |
| 10 | ACUGGAAAGAAAAAGAAGA | UCUUCUUUUUCUUUCCAGU | Chn | [682-700] |
| 11 | CCAGUGCUGUUAGAAUAAA | UUUAUUCUAACAGCACUGG | | [1155-1173] |
| 12 | CUUUCUUGUCUCCAAGGAA | UUCCUUGGAGACAAGAAAG | | [343-361] |
| 13 | AGAACUUGAGUGUGGCCGA | UCGGCCACACUCAAGUUCU | | [764-782] |
| 14 | GACAGUUGUGUUGUGCCAA | UUGGCACAACACAACUGUC | | [475-493] |
| 15 | AGGUGAUCAUUUUAAACUU | AAGUUUAAAAUGAUCACCU | | [708-726] |
| 16 | CAAGCUACCGGAAGUGAUG | CAUCACUUCCGGUAGCUUG | | [405-423] |
| 17 | CAGAAUUUCAUCACCCAAU | AUUGGGUGAUGAAAUUCUG | | [1014-1032] |
| 18 | CACUGGAAAGAAAAAGAAG | CUUCUUUUUCUUUCCAGUG | Chn | [681-699] |
| 19 | AGAAGAAAACCCGAGGUGA | UCACCUCGGGUUUUCUUCU | | [695-713] |
| 20 | UUUCUUGUCUCCAAGGAAA | UUUCCUUGGAGACAAGAAA | | [344-362] |
| 21 | GAAUGACAACUUCCAGGAU | AUCCUGGAAGUUGUCAUUC | Ms | [600-618] |
| 22 | AAAGCUUUCUUGUCUCCAA | UUGGAGACAAGAAAGCUUU | | [339-357] |
| 23 | AGUACAAGCUACCGGAAGU | ACUUCCGGUAGCUUGUACU | | [401-419] |
| 24 | CAAGAUUUACACCUCCACA | UGUGGAGGUGUAAAUCUUG | | [280-298] |
| 25 | GUAAGAACACCGUGACCAC | GUGGUCACGGUGUUCUUAC | | [230-248] |
| 26 | AUGACAACUUCCAGGAUGA | UCAUCCUGGAAGUUGUCAU | Ms | [602-620] |
| 27 | CCUGGAGAAUGACAACUUC | GAAGUUGUCAUUCUCCAGG | | [594-612] |
| 28 | GUGUUGUGCCAAUGGUGGA | UCCACCAUUGGCACAACAC | | [482-500] |
| 29 | GGUGGAGAAGAAAACUUCG | CGAAGUUUUCUUCUCCACC | | [495-513] |
| 30 | GUGCUGUUAGAAUAAAAAG | CUUUUUAUUCUAACAGCAC | | [1158-1176] |
| 31 | UCUUUAAAAUGACCCAUUU | AAAUGGGUCAUUUUAAAGA | | [250-268] |
| 32 | UUGGAGGAGCAGAACUUGA | UCAAGUUCUGCUCCUCCAA | | [754-772] |
| 33 | UCUGAAGUGGACUGUGUGA | UCACACAGUCCACUUCAGA | | [939-957] |
| 34 | AACUGUGUCUUAUCUGCCA | UGGCAGAUAAGACACAGUU | | [1085-1103] |
| 35 | UAAGAACACCGUGACCACA | UGUGGUCACGGUGUUCUUA | | [231-249] |
| 36 | AAGAUUUCUUGUAAGAACA | UGUUCUUACAAGAAAUCUU | | [220-238] |
| 37 | AGGACCAAAGGAAAAUAAG | CUUAUUUUCCUUUGGUCCU | | [204-222] |
| 38 | GUGCCAAUGGUGGAGAAGA | UCUUCUCCACCAUUGGCAC | | [487-505] |
| 39 | UUUCGUGGCUCCCACAAGA | UCUUGUGGGAGCCACGAAA | | [266-284] |
| 40 | ACAGUUGUGUUGUGCCAAU | AUUGGCACAACACAACUGU | | [476-494] |
| 41 | UUCACAAGGACCAAAGGAA | UUCCUUUGGUCCUUGUGAA | | [198-216] |
| 42 | AAUAAGAUUUCUUGUAAGA | UCUUACAAGAAAUCUUAUU | | [217-235] |
| 43 | CCAAAGGAAAAUAAGAUUU | AAAUCUUAUUUUCCUUUGG | | [208-226] |
| 44 | AUUUACACCUCCACACUGA | UCAGUGUGGAGGUGUAAAU | | [284-302] |
| 45 | AAAACAUGGCGAAAAGCUU | AAGCUUUUCGCCAUGUUUU | | [327-345] |
| 46 | GUUUGAUGACGAUGCGGAC | GUCCGCAUCGUCAUCAAAC | | [663-681] |
| 47 | ACCAAAGGAAAAUAAGAUU | AAUCUUAUUUUCCUUUGGU | | [207-225] |
| 48 | AAGAACACCGUGACCACAU | AUGUGGUCACGGUGUUCUU | | [232-250] |
| 49 | GAAACGCCACGUAAUGAGU | ACUCAUUACGUGGCGUUUC | | [359-377] |
| 50 | UGGAGGAGCAGAACUUGAG | CUCAAGUUCUGCUCCUCCA | | [755-773] |
| 51 | GAACACCGUGACCACAUCU | AGAUGUGGUCACGGUGUUC | | [234-252] |
| 52 | UAAACUUCGCUUCCGAAAA | UUUUCGGAAGCGAAGUUUA | GP | [720-738] |
| 53 | UGGCGAAAAGCUUUCUUGU | ACAAGAAAGCUUUUCGCCA | | [333-351] |
| 54 | UAUAAAACAUGGCGAAAAG | CUUUUCGCCAUGUUUUAUA | | [324-342] |
| 55 | GGAAAAUAAGAUUUCUUGU | ACAAGAAAUCUUAUUUUCC | | [213-231] |
| 56 | AGGAAACGCCACGUAAUGA | UCAUUACGUGGCGUUUCCU | | [357-375] |
| 57 | UCUUGUAAGAACACCGUGA | UCACGGUGUUCUUACAAGA | | [226-244] |
| 58 | GUCUUAUCUGCCAGGAAAG | CUUUCCUGGCAGAUAAGAC | | [1091-1109] |
| 59 | UGUGAUCACAGCAUCUUCA | UGAAGAUGCUGUGAUCACA | | [183-201] |
| 60 | AGAGACUGCCUCAGUUUGA | UCAAACUGAGGCAGUCUCU | | [650-668] |
| 61 | AAAUAAGAUUUCUUGUAAG | CUUACAAGAAAUCUUAUUU | | [216-234] |
| 62 | CUUCACAAGGACCAAAGGA | UCCUUUGGUCCUUGUGAAG | | [197-215] |
| 63 | AGCCAGCCACUUUCUUUGA | UCAAAGAAAGUGGCUGGCU | | [114-132] |
| 64 | CAUUUCGUGGCUCCCACAA | UUGUGGGAGCCACGAAAUG | | [264-282] |
| 65 | UCUUAUCUGCCAGGAAAGA | UCUUUCCUGGCAGAUAAGA | | [1092-1110] |
| 66 | AUGCGGACACUGGAAAGAA | UUCUUUCCAGUGUCCGCAU | Chn | [674-692] |
| 67 | GAAAAUAAGAUUUCUUGUA | UACAAGAAAUCUUAUUUUC | | [214-232] |
| 68 | CUGUUGGAGGAGCAGAACU | AGUUCUGCUCCUCCAACAG | | [751-769] |
| 69 | AGAAAAAGAAGAAAACCCG | CGGGUUUUCUUCUUUUUCU | | [689-707] |
| 70 | AUUUCAUCACCCAAUGCAG | CUGCAUUGGGUGAUGAAAU | | [1018-1036] |
| 71 | GAGCAGAACUUGAGUGUGG | CCACACUCAAGUUCUGCUC | | [760-778] |
| 72 | UGAUCACAGCAUCUUCACA | UGUGAAGAUGCUGUGAUCA | | [185-203] |
| 73 | ACCAGGUGUCUGAAGUGGA | UCCACUUCAGACACCUGGU | Ms | [931-949] |
| 74 | UUUAAACUUCGCUUCCGAA | UUCGGAAGCGAAGUUUAAA | GP, Chn | [718-736] |
| 75 | CAUUCACCCAACAAAACUG | CAGUUUUGUUGGGUGAAUG | | [1071-1089] |
| 76 | GUUGGAGGAGCAGAACUUG | CAAGUUCUGCUCCUCCAAC | | [753-771] |
| 77 | AGUUUGAUGACGAUGCGGA | UCCGCAUCGUCAUCAAACU | | [662-680] |
| 78 | GAUGCGGACACUGGAAAGA | UCUUUCCAGUGUCCGCAUC | Chn | [673-691] |
| 79 | GUCUGAAGUGGACUGUGUG | CACACAGUCCACUUCAGAC | | [938-956] |
| 80 | CUCAUUCACCCAACAAAAC | GUUUUGUUGGGUGAAUGAG | | [1069-1087] |
| 81 | GGUGAUCAUUUUAAACUUC | GAAGUUUAAAAUGAUCACC | | [709-727] |
| 82 | AAACAUGGCGAAAAGCUUU | AAAGCUUUUCGCCAUGUUU | | [328-346] |
| 83 | AGAAUUUCAUCACCCAAUG | CAUUGGGUGAUGAAAUUCU | | [1015-1033] |
| 84 | GAUUUACACCUCCACACUG | CAGUGUGGAGGUGUAAAUC | | [283-301] |
| 85 | UAUCUGCCAGGAAAGACCA | UGGUCUUUCCUGGCAGAUA | | [1095-1113] |
| 86 | ACUUUCAGGCCCUGUUGGA | UCCAACAGGGCCUGAAAGU | | [740-758] |
| 87 | AAUGGUGGAGAAGAAAACU | AGUUUUCUUCUCCACCAUU | | [492-510] |
| 88 | UACAAGCUACCGGAAGUGA | UCACUUCCGGUAGCUUGUA | | [403-421] |
| 89 | AUCACAGCAUCUUCACAAG | CUUGUGAAGAUGCUGUGAU | | [187-205] |
| 90 | GAUCAUUUUAAACUUCGCU | AGCGAAGUUUAAAAUGAUC | | [712-730] |
| 91 | AAUUUCAUCACCCAAUGCA | UGCAUUGGGUGAUGAAAUU | | [1017-1035] |
| 92 | CUUCGCUUCCGAAAAAACU | AGUUUUUUCGGAAGCGAAG | GP | [724-742] |
| 93 | UUUUAAACUUCGCUUCCGA | UCGGAAGCGAAGUUUAAAA | | [717-735] |
| 94 | GUUGUGUUGUGCCAAUGGU | ACCAUUGGCACAACACAAC | | [479-497] |
| 95 | UGUCUCCAAGGAAACGCCA | UGGCGUUUCCUUGGAGACA | | [349-367] |
| 96 | AAGGAAACGCCACGUAAUG | CAUUACGUGGCGUUUCCUU | | [356-374] |
| 97 | AAACCCGAGGUGAUCAUUU | AAAUGAUCACCUCGGGUUU | | [701-719] |
| 98 | UGAAGUGGACUGUGUGAGC | GCUCACACAGUCCACUUCA | | [941-959] |
| 99 | AAGCUUUCUUGUCUCCAAG | CUUGGAGACAAGAAAGCUU | | [340-358] |
| 100 | AACACCGUGACCACAUCUU | AAGAUGUGGUCACGGUGUU | | [235-253] |
| 101 | CUUUGUGAUCACAGCAUCU | AGAUGCUGUGAUCACAAAG | | [180-198] |
| 102 | UUAAACUUCGCUUCCGAAA | UUUCGGAAGCGAAGUUUAA | GP, Chn | [719-737] |
| 103 | UUGUGUUGUGCCAAUGGUG | CACCAUUGGCACAACACAA | | [480-498] |
| 104 | AAAGAAGAAAACCCGAGGU | ACCUCGGGUUUUCUUCUUU | | [693-711] |
| 105 | GACUGCCUCAGUUUGAUGA | UCAUCAAACUGAGGCAGUC | | [653-671] |
| 106 | UCGUGGCUCCCACAAGAUU | AAUCUUGUGGGAGCCACGA | | [268-286] |
| 107 | UCAUUCACCCAACAAAACU | AGUUUUGUUGGGUGAAUGA | | [1070-1088] |
| 108 | AGGAAAGACCAGCCUCACU | AGUGAGGCUGGUCUUUCCU | | [1103-1121] |
| 109 | AAAAAGAAGAAAACCCGAG | CUCGGGUUUUCUUCUUUUU | | [691-709] |
| 110 | AGAUUUACACCUCCACACU | AGUGUGGAGGUGUAAAUCU | | [282-300] |
| 111 | CACUAGGAACCUCGGAUUG | CAAUCCGAGGUUCCUAGUG | | [87-105] |
| 112 | GUUAGAAUAAAAAGCCUCG | CGAGGCUUUUUAUUCUAAC | | [1163-1181] |
| 113 | UGUGUCUUAUCUGCCAGGA | UCCUGGCAGAUAAGACACA | | [1088-1106] |
| 114 | UCUUUGACAGUCCAGCCCA | UGGGCUGGACUGUCAAAGA | | [126-144] |
| 115 | GUUGUGCCAAUGGUGGAGA | UCUCCACCAUUGGCACAAC | | [484-502] |
| 116 | CAUGGCGAAAAGCUUUCUU | AAGAAAGCUUUUCGCCAUG | | [331-349] |
| 117 | AAAAGAAGAAAACCCGAGG | CCUCGGGUUUUCUUCUUUU | | [692-710] |
| 118 | AAGGAAAAUAAGAUUUCUU | AAGAAAUCUUAUUUUCCUU | | [211-229] |
| 119 | AUAAGAUUUCUUGUAAGAA | UUCUUACAAGAAAUCUUAU | | [218-236] |
| 120 | CAGCAUCUUCACAAGGACC | GGUCCUUGUGAAGAUGCUG | | [191-209] |
| 121 | AAACUUCGGUUCGCUCCCA | UGGGAGCGAACCGAAGUUU | | [506-524] |
| 122 | GAGGAGCAGAACUUGAGUG | CACUCAAGUUCUGCUCCUC | | [757-775] |
| 123 | AGUUUCUUCCGACAGUUGU | ACAACUGUCGGAAGAAACU | | [465-483] |
| 124 | GUUUCUUCCGACAGUUGUG | CACAACUGUCGGAAGAAAC | | [466-484] |
| 125 | UUAAAAUGACCCAUUUCGU | ACGAAAUGGGUCAUUUUAA | | [253-271] |
| 126 | AUCUUUAAAAUGACCCAUU | AAUGGGUCAUUUUAAAGAU | | [249-267] |
| 127 | AGAAAACCCGAGGUGAUCA | UGAUCACCUCGGGUUUUCU | | [698-716] |
| 128 | GCGAAAAGCUUUCUUGUCU | AGACAAGAAAGCUUUUCGC | | [335-353] |
| 129 | CGAAAAGCUUUCUUGUCUC | GAGACAAGAAAGCUUUUCG | | [336-354] |
| 130 | UCUUCACAAGGACCAAAGG | CCUUUGGUCCUUGUGAAGA | | [196-214] |
| 131 | AGGAAAAUAAGAUUUCUUG | CAAGAAAUCUUAUUUUCCU | | [212-230] |
| 132 | AAACUUUCAGGCCCUGUUG | CAACAGGGCCUGAAAGUUU | | [738-756] |
| 133 | AAAACCCGAGGUGAUCAUU | AAUGAUCACCUCGGGUUUU | | [700-718] |
| 134 | AGUUGUGUUGUGCCAAUGG | CCAUUGGCACAACACAACU | | [478-496] |
| 135 | GCAGAAGUACAAGCUACCG | CGGUAGCUUGUACUUCUGC | | [396-414] |
| 136 | CAGUUUCUUCCGACAGUUG | CAACUGUCGGAAGAAACUG | | [464-482] |
| 137 | GUGGAGAAGAAAACUUCGG | CCGAAGUUUUCUUCUCCAC | | [496-514] |
| 138 | AAAGGAAAAUAAGAUUUCU | AGAAAUCUUAUUUUCCUUU | | [210-228] |
| 139 | AAAAAACUUUCAGGCCCUG | CAGGGCCUGAAAGUUUUUU | | [735-753] |
| 140 | AAAGAAAAAGAAGAAAACC | GGUUUUCUUCUUUUUCUUU | | [687-705] |
| 141 | AAGAGACUGCCUCAGUUUG | CAAACUGAGGCAGUCUCUU | | [649-667] |
| 142 | CUUCCGACAGUUGUGUUGU | ACAACACAACUGUCGGAAG | | [470-488] |
| 143 | UGUUGUGCCAAUGGUGGAG | CUCCACCAUUGGCACAACA | | [483-501] |
| 144 | AGUUCAGCUUUCUGCAGAG | CUCUGCAGAAAGCUGAACU | | [65-83] |
| 145 | UGUCUGAAGUGGACUGUGU | ACACAGUCCACUUCAGACA | | [937-955] |
| 146 | UGCCUUUGUGAUCACAGCA | UGCUGUGAUCACAAAGGCA | | [177-195] |
| 147 | AAGCCAGCCACUUUCUUUG | CAAAGAAAGUGGCUGGCUU | | [113-131] |
| 148 | AACGCCACGUAAUGAGUCA | UGACUCAUUACGUGGCGUU | | [361-379] |
| 149 | ACUUUCUUUGACAGUCCAG | CUGGACUGUCAAAGAAAGU | | [122-140] |
| 150 | UGACAACUUCCAGGAUGAC | GUCAUCCUGGAAGUUGUCA | Ms | [603-621] |
| 151 | GUGAUCACAGCAUCUUCAC | GUGAAGAUGCUGUGAUCAC | | [184-202] |
| 152 | UUCCGACAGUUGUGUUGUG | CACAACACAACUGUCGGAA | | [471-489] |
| 153 | AAAACUGUGUCUUAUCUGC | GCAGAUAAGACACAGUUUU | | [1083-1101] |
| 154 | ACGUAAUGAGUCAAAGCUG | CAGCUUUGACUCAUUACGU | | [367-385] |
| 155 | GUAAUGAGUCAAAGCUGUG | CACAGCUUUGACUCAUUAC | | [369-387] |
| 156 | UAAGAUUUCUUGUAAGAAC | GUUCUUACAAGAAAUCUUA | | [219-237] |
| 157 | CAAAGGAAAAUAAGAUUUC | GAAAUCUUAUUUUCCUUUG | | [209-227] |
| 158 | AUUCACCCAACAAAACUGU | ACAGUUUUGUUGGGUGAAU | | [1072-1090] |
| 159 | AAGAAAAAGAAGAAAACCC | GGGUUUUCUUCUUUUUCUU | | [688-706] |
| 160 | CUUGUAAGAACACCGUGAC | GUCACGGUGUUCUUACAAG | | [227-245] |
| 161 | AAGUGGACUGUGUGAGCCU | AGGCUCACACAGUCCACUU | | [943-961] |
| 162 | UUCUUGUCUCCAAGGAAAC | GUUUCCUUGGAGACAAGAA | | [345-363] |
| 163 | UGUUAGAAUAAAAAGCCUC | GAGGCUUUUUAUUCUAACA | | [1162-1180] |
| 164 | AGGUGUCUGAAGUGGACUG | CAGUCCACUUCAGACACCU | | [934-952] |
| 165 | AGACCAGGUGUCUGAAGUG | CACUUCAGACACCUGGUCU | Ms | [929-947] |
| 166 | UUAGAAUAAAAAGCCUCGU | ACGAGGCUUUUUAUUCUAA | | [1164-1182] |
| 167 | UUCGCUUCCGAAAAAACUU | AAGUUUUUUCGGAAGCGAA | GP | [725-743] |
| 168 | ACUUCGCUUCCGAAAAAAC | GUUUUUUCGGAAGCGAAGU | GP | [723-741] |
| 169 | AGACUGCCUCAGUUUGAUG | CAUCAAACUGAGGCAGUCU | | [652-670] |
| 170 | UGUGCCAAUGGUGGAGAAG | CUUCUCCACCAUUGGCACA | | [486-504] |
| 171 | UUGUGAUCACAGCAUCUUC | GAAGAUGCUGUGAUCACAA | | [182-200] |
| 172 | CUGCCUUUGUGAUCACAGC | GCUGUGAUCACAAAGGCAG | | [176-194] |
| 173 | CAGGUGUCUGAAGUGGACU | AGUCCACUUCAGACACCUG | | [933-951] |
| 174 | CAUUUUAAACUUCGCUUCC | GGAAGCGAAGUUUAAAAUG | | [715-733] |
| 175 | UGUAAGAACACCGUGACCA | UGGUCACGGUGUUCUUACA | | [229-247] |
| 176 | GAAUUUCAUCACCCAAUGC | GCAUUGGGUGAUGAAAUUC | | [1016-1034] |
| 177 | AAGUACAAGCUACCGGAAG | CUUCCGGUAGCUUGUACUU | | [400-418] |
| 178 | AGAAAGACAGAAUUUCAUC | GAUGAAAUUCUGUCUUUCU | | [1007-1025] |
| 179 | CAGUUUGAUGACGAUGCGG | CCGCAUCGUCAUCAAACUG | | [661-679] |
| 180 | UAACUACCUGACGGCCUGU | ACAGGCCGUCAGGUAGUUA | | [789-807] |
| 181 | CAGCAGUUUCUUCCGACAG | CUGUCGGAAGAAACUGCUG | | [461-479] |
| 182 | AUCAUUUUAAACUUCGCUU | AAGCGAAGUUUAAAAUGAU | | [713-731] |
| 183 | AUCUUCACAAGGACCAAAG | CUUUGGUCCUUGUGAAGAU | | [195-213] |
| 184 | AGAAGAAAACUUCGGUUCG | CGAACCGAAGUUUUCUUCU | | [500-518] |
| 185 | GUGUCUGAAGUGGACUGUG | CACAGUCCACUUCAGACAC | | [936-954] |
| 186 | UUCGUGGCUCCCACAAGAU | AUCUUGUGGGAGCCACGAA | | [267-285] |
| 187 | ACAUGGCGAAAAGCUUUCU | AGAAAGCUUUUCGCCAUGU | | [330-348] |
| 188 | GGCGAAAAGCUUUCUUGUC | GACAAGAAAGCUUUUCGCC | | [334-352] |
| 189 | CUUUCUUUGACAGUCCAGC | GCUGGACUGUCAAAGAAAG | | [123-141] |
| 190 | AAGAAAACUUCGGUUCGCU | AGCGAACCGAAGUUUUCUU | | [502-520] |
| 191 | CUGAAGUGGACUGUGUGAG | CUCACACAGUCCACUUCAG | | [940-958] |
| 192 | AAGACAGAAUUUCAUCACC | GGUGAUGAAAUUCUGUCUU | | [1010-1028] |
| 193 | UUUACACCUCCACACUGAG | CUCAGUGUGGAGGUGUAAA | | [285-303] |
| 194 | GAGAAUGACAACUUCCAGG | CCUGGAAGUUGUCAUUCUC | Ms, Chn | [598-616] |
| 195 | CUUCCGAAAAAACUUUCAG | CUGAAAGUUUUUUCGGAAG | | [729-747] |
| 196 | UGCUGUUAGAAUAAAAAGC | GCUUUUUAUUCUAACAGCA | | [1159-1177] |
| 197 | AGCCACUUUCUUUGACAGU | ACUGUCAAAGAAAGUGGCU | | [118-136] |
| 198 | AAUGACAACUUCCAGGAUG | CAUCCUGGAAGUUGUCAUU | Ms | [601-619] |
| 199 | CCGAAAAAACUUUCAGGCC | GGCCUGAAAGUUUUUUCGG | | [732-750] |
| 200 | CAGAAGUACAAGCUACCGG | CCGGUAGCUUGUACUUCUG | | [397-415] |
| 201 | AAAGACCAGCCUCACUCCU | AGGAGUGAGGCUGGUCUUU | | [1106-1124] |
| 202 | CAAAACUGUGUCUUAUCUG | CAGAUAAGACACAGUUUUG | | [1082-1100] |
| 203 | AGAAAACUUCGGUUCGCUC | GAGCGAACCGAAGUUUUCU | | [503-521] |
| 204 | UGUGUUGUGCCAAUGGUGG | CCACCAUUGGCACAACACA | | [481-499] |
| 205 | UGGCUCCCACAAGAUUUAC | GUAAAUCUUGUGGGAGCCA | | [271-289] |
| 206 | UUUCUGCAGAGGGCCACUA | UAGUGGCCCUCUGCAGAAA | | [73-91] |
| 207 | UAGAAUAAAAAGCCUCGUG | CACGAGGCUUUUUAUUCUA | | [1165-1183] |
| 208 | GCUGCCUUUGUGAUCACAG | CUGUGAUCACAAAGGCAGC | | [175-193] |
| 209 | CUUUAAAAUGACCCAUUUC | GAAAUGGGUCAUUUUAAAG | | [251-269] |
| 210 | AAGAUUUACACCUCCACAC | GUGUGGAGGUGUAAAUCUU | | [281-299] |
| 211 | AAAAGCUUUCUUGUCUCCA | UGGAGACAAGAAAGCUUUU | | [338-356] |
| 212 | GAUUUCUUGUAAGAACACC | GGUGUUCUUACAAGAAAUC | | [222-240] |
| 213 | AAGCUACCGGAAGUGAUGG | CCAUCACUUCCGGUAGCUU | | [406-424] |
| 214 | AGCAGAACUUGAGUGUGGC | GCCACACUCAAGUUCUGCU | | [761-779] |
| 215 | AAGAAGAAAACCCGAGGUG | CACCUCGGGUUUUCUUCUU | | [694-712] |
| 216 | CUGUGUCUUAUCUGCCAGG | CCUGGCAGAUAAGACACAG | | [1087-1105] |
| 217 | AUCUGCCAGGAAAGACCAG | CUGGUCUUUCCUGGCAGAU | | [1096-1114] |
| 218 | UCACAGCAUCUUCACAAGG | CCUUGUGAAGAUGCUGUGA | | [188-206] |
| 219 | AUGGCGAAAAGCUUUCUUG | CAAGAAAGCUUUUCGCCAU | | [332-350] |
| 220 | CUCCCACAAGAUUUACACC | GGUGUAAAUCUUGUGGGAG | | [274-292] |
| 221 | AACAAAACUGUGUCUUAUC | GAUAAGACACAGUUUUGUU | | [1080-1098] |
| 222 | ACUGUGUCUUAUCUGCCAG | CUGGCAGAUAAGACACAGU | | [1086-1104] |
| 223 | AGAAUAAAAAGCCUCGUGC | GCACGAGGCUUUUUAUUCU | | [1166-1184] |
| 224 | AAAAACUUUCAGGCCCUGU | ACAGGGCCUGAAAGUUUUU | | [736-754] |
| 225 | CUCAGUUUGAUGACGAUGC | GCAUCGUCAUCAAACUGAG | | [659-677] |
| 226 | UUGUAAGAACACCGUGACC | GGUCACGGUGUUCUUACAA | | [228-246] |
| 227 | AGCUUUCUUGUCUCCAAGG | CCUUGGAGACAAGAAAGCU | | [341-359] |
| 228 | UUUGAUGACGAUGCGGACA | UGUCCGCAUCGUCAUCAAA | | [664-682] |
| 229 | AAGAAAACCCGAGGUGAUC | GAUCACCUCGGGUUUUCUU | | [697-715] |
| 230 | ACUGGGAGUUCAGCUUUCU | AGAAAGCUGAACUCCCAGU | | [59-77] |
| 231 | AUUUCGUGGCUCCCACAAG | CUUGUGGGAGCCACGAAAU | | [265-283] |
| 232 | CUGGGAGUUCAGCUUUCUG | CAGAAAGCUGAACUCCCAG | | [60-78] |
| 233 | UUUCUUGUAAGAACACCGU | ACGGUGUUCUUACAAGAAA | | [224-242] |
| 234 | UAAAACAUGGCGAAAAGCU | AGCUUUUCGCCAUGUUUUA | | [326-344] |
| 235 | UUGUCUCCAAGGAAACGCC | GGCGUUUCCUUGGAGACAA | | [348-366] |
| 236 | UCUUGUCUCCAAGGAAACG | CGUUUCCUUGGAGACAAGA | | [346-364] |
| 237 | AAAGACAGAAUUUCAUCAC | GUGAUGAAAUUCUGUCUUU | | [1009-1027] |
| 238 | GAAAAGCUUUCUUGUCUCC | GGAGACAAGAAAGCUUUUC | | [337-355] |
| 239 | AUUUCUUGUAAGAACACCG | CGGUGUUCUUACAAGAAAU | | [223-241] |
| 240 | GAAAACUUCGGUUCGCUCC | GGAGCGAACCGAAGUUUUC | | [504-522] |
| 241 | AUAAAACAUGGCGAAAAGC | GCUUUUCGCCAUGUUUUAU | | [325-343] |
| 242 | UUCUGUGCUGUCUGUGGCU | AGCCACAGACAGCACAGAA | | [838-856] |
| 243 | UAAAAUGACCCAUUUCGUG | CACGAAAUGGGUCAUUUUA | | [254-272] |
| 244 | UCACUCCUGGGAACUGUCU | AGACAGUUCCCAGGAGUGA | | [1117-1135] |
| 245 | UUUCAUCACCCAAUGCAGG | CCUGCAUUGGGUGAUGAAA | | [1019-1037] |
| 246 | UCUGUGCUGUCUGUGGCUU | AAGCCACAGACAGCACAGA | | [839-857] |
| 247 | CACAUCUUUAAAAUGACCC | GGGUCAUUUUAAAGAUGUG | | [246-264] |
| 248 | ACUGCCUCAGUUUGAUGAC | GUCAUCAAACUGAGGCAGU | | [654-672] |
| 249 | CCUCAGUUUGAUGACGAUG | CAUCGUCAUCAAACUGAGG | | [658-676] |
| 250 | CUGGAGAAUGACAACUUCC | GGAAGUUGUCAUUCUCCAG | | [595-613] |
| 251 | UUCUUGUAAGAACACCGUG | CACGGUGUUCUUACAAGAA | | [225-243] |
| 252 | UUCCGAAAAAACUUUCAGG | CCUGAAAGUUUUUUCGGAA | | [730-748] |
| 253 | AAACUGUGUCUUAUCUGCC | GGCAGAUAAGACACAGUUU | | [1084-1102] |
| 254 | UUAUCUGCCAGGAAAGACC | GGUCUUUCCUGGCAGAUAA | | [1094-1112] |
| 255 | AACUUUCAGGCCCUGUUGG | CCAACAGGGCCUGAAAGUU | | [739-757] |
| 256 | UUUGUGAUCACAGCAUCUU | AAGAUGCUGUGAUCACAAA | | [181-199] |
| 257 | AAAACUUCGGUUCGCUCCC | GGGAGCGAACCGAAGUUUU | | [505-523] |
| 258 | UAAUGAGUCAAAGCUGUGG | CCACAGCUUUGACUCAUUA | | [370-388] |
| 259 | ACUAGGAACCUCGGAUUGC | GCAAUCCGAGGUUCCUAGU | | [88-106] |
| 260 | UCCGACAGUUGUGUUGUGC | GCACAACACAACUGUCGGA | | [472-490] |
| 261 | AACUGGGAGUUCAGCUUUC | GAAAGCUGAACUCCCAGUU | | [58-76] |
| 262 | UUCAGAAAGACAGAAUUUC | GAAAUUCUGUCUUUCUGAA | | [1004-1022] |
| 263 | CUGCCUCAGUUUGAUGACG | CGUCAUCAAACUGAGGCAG | | [655-673] |
| 264 | UCCGAAAAAACUUUCAGGC | GCCUGAAAGUUUUUUCGGA | | [731-749] |
| 265 | CUUAUCUGCCAGGAAAGAC | GUCUUUCCUGGCAGAUAAG | | [1093-1111] |
| 266 | UUCUUCCGACAGUUGUGUU | AACACAACUGUCGGAAGAA | | [468-486] |
| 267 | CUUGUCUCCAAGGAAACGC | GCGUUUCCUUGGAGACAAG | | [347-365] |
| 268 | ACAACUUCCAGGAUGACCC | GGGUCAUCCUGGAAGUUGU | Rat, Ms | [605-623] |
| 269 | UCUUCCGACAGUUGUGUUG | CAACACAACUGUCGGAAGA | | [469-487] |
| 270 | UUUCUUCCGACAGUUGUGU | ACACAACUGUCGGAAGAAA | | [467-485] |
| 271 | AACAUGGCGAAAAGCUUUC | GAAAGCUUUUCGCCAUGUU | | [329-347] |
| 272 | UGGGAGUUCAGCUUUCUGC | GCAGAAAGCUGAACUCCCA | | [61-79] |
| 273 | AAUGAGUCAAAGCUGUGGC | GCCACAGCUUUGACUCAUU | | [371-389] |
| 274 | UUCACCCAACAAAACUGUG | CACAGUUUUGUUGGGUGAA | | [1073-1091] |
| 275 | GUUCAGCUUUCUGCAGAGG | CCUCUGCAGAAAGCUGAAC | | [66-84] |
| 276 | GACAACUUCCAGGAUGACC | GGUCAUCCUGGAAGUUGUC | Rat, Ms | [604-622] |
| 277 | AAUGACCCAUUUCGUGGCU | AGCCACGAAAUGGGUCAUU | | [257-275] |
| 278 | AAAUGACCCAUUUCGUGGC | GCCACGAAAUGGGUCAUUU | | [256-274] |
| 279 | AAACGCCACGUAAUGAGUC | GACUCAUUACGUGGCGUUU | | [360-378] |
| 280 | UUCAUCACCCAAUGCAGGG | CCCUGCAUUGGGUGAUGAA | | [1020-1038] |
| 281 | UUGAUGACGAUGCGGACAC | GUGUCCGCAUCGUCAUCAA | | [665-683] |
| 282 | UUUAAAAUGACCCAUUUCG | CGAAAUGGGUCAUUUUAAA | | [252-270] |
| 283 | AAAAUGACCCAUUUCGUGG | CCACGAAAUGGGUCAUUUU | | [255-273] |
| 284 | UUUCUUUGACAGUCCAGCC | GGCUGGACUGUCAAAGAAA | | [124-142] |
| 285 | AUUUUAAACUUCGCUUCCG | CGGAAGCGAAGUUUAAAAU | | [716-734] |
| 286 | UUACACCUCCACACUGAGG | CCUCAGUGUGGAGGUGUAA | | [286-304] |
| 287 | AUGAGUCAAAGCUGUGGCG | CGCCACAGCUUUGACUCAU | | [372-390] |
| 288 | UUCUUUGACAGUCCAGCCC | GGGCUGGACUGUCAAAGAA | | [125-143] |
| 289 | UUCAGCUUUCUGCAGAGGG | CCCUCUGCAGAAAGCUGAA | | [67-85] |
| 290 | AUGACCCAUUUCGUGGCUC | GAGCCACGAAAUGGGUCAU | | [258-276] |

TABLE B11
LGALS3 19-mer oligomer pairs lectin, galactoside-binding, soluble, 3

| | | | | Human- | |
|---|---|---|---|---|---|
| | | | Other | 115430222: | Human- |
| No. | Sense | AntiSense | Sp | 152-904 | 115430224 |

| | | | | | |
|---|---|---|---|---|---|
| 1 | UGCCUUAUAACCUGCCUUU | AAAGGCAGGUUAUAAGGCA | | [498-516] | [1186-1204] |
| 2 | CAAUACAAAGCUGGAUAAU | AUUAUCCAGCUUUGUAUUG | | [670-688] | [1358-1376] |
| 3 | GUGCCUUAUAACCUGCCUU | AAGGCAGGUUAUAAGGCAC | | [497-515] | [1185-1203] |
| 4 | GGGAAUUUCUGGUGACAUA | UAUGUCACCAGAAAUUCCC | | [853-871] | [1541-1559] |
| 5 | ACAUUCAUCAAUAUCCCUC | GAGGGAUAUUGAUGAAUGU | | [990-1008] | [1678-1696] |
| 6 | CAAAAUACAAGUACUGGUU | AACCAGUACUUGUAUUUUG | | [745-763] | [1433-1451] |
| 7 | CCAUUCAAAAUACAAGUAC | GUACUUGUAUUUUGAAUGG | | [740-758] | [1428-1446] |
| 8 | GGAAGAAAGACAGUCGGUU | AACCGACUGUCUUUCUUCC | | [700-718] | [1388-1406] |
| 9 | GGAAAAUGGCAGACAAUUU | AAAUUGUCUGCCAUUUUCC | | [147-165] | [182-200] |
| 10 | CCAUGAUAUAAUCUGAAAG | CUUUCAGAUUAUAUCAUGG | | [894-912] | [1582-1600] |
| 11 | GCAGUACAAUCAUCGGGUU | AACCCGAUGAUUGUACUGC | | [808-826] | [1496-1514] |
| 12 | GAGAGUCAUUGUUUGCAAU | AUUGCAAACAAUGACUCUC | | [655-673] | [1343-1361] |
| 13 | GAAAAUGGCAGACAAUUUU | AAAAUUGUCUGCCAUUUUC | | [148-166] | [183-201] |
| 14 | GCGUUAUCUGGGUCUGGAA | UUCCAGACCCAGAUAACGC | | [179-197] | [867-885] |
| 15 | GGGUUAAAAAACUCAAUGA | UCAUUGAGUUUUUUAACCC | | [822-840] | [1510-1528] |
| 16 | CGGGUUAAAAAACUCAAUG | CAUUGAGUUUUUUAACCCG | | [821-839] | [1509-1527] |
| 17 | UCAAAAUACAAGUACUGGU | ACCAGUACUUGUAUUUUGA | | [744-762] | [1432-1450] |
| 18 | CAAGGUUGCAGUGAAUGAU | AUCAUUCACUGCAACCUUG | | [778-796] | [1466-1484] |
| 19 | UCUAAACCUUACAUGUGUA | UACACAUGUAAGGUUUAGA | | [938-956] | [1626-1644] |
| 20 | CCAGUGCUUCAUAUACCAU | AUGGUAUAUGAAGCACUGG | | [879-897] | [1567-1585] |
| 21 | GACCUCACCAGUGCUUCAU | AUGAAGCACUGGUGAGGUC | | [872-890] | [1560-1578] |
| 22 | CUUUAACCCACGCUUCAAU | AUUGAAGCGUGGGUUAAAG | | [625-643] | [1313-1331] |
| 23 | UCCACUUUAACCCACGCUU | AAGCGUGGGUUAAAGUGGA | | [621-639] | [1309-1327] |
| 24 | GCCUUCCACUUUAACCCAC | GUGGGUUAAAGUGGAAGGC | | [617-635] | [1305-1323] |
| 25 | CGUUAUCUGGGUCUGGAAA | UUUCCAGACCCAGAUAACG | | [180-198] | [868-886] |
| 26 | ACCUCACCAGUGCUUCAUA | UAUGAAGCACUGGUGAGGU | | [873-891] | [1561-1579] |
| 27 | AAGAAAGACAGUCGGUUUU | AAAACCGACUGUCUUUCUU | | [702-720] | [1390-1408] |
| 28 | GGGAAGAAAGACAGUCGGU | ACCGACUGUCUUUCUUCCC | | [699-717] | [1387-1405] |
| 29 | CCACUUUAACCCACGCUUC | GAAGCGUGGGUUAAAGUGG | | [622-640] | [1310-1328] |
| 30 | CCUCGCAUGCUGAUAACAA | UUGUUAUCAGCAUGCGAGG | | [533-551] | [1221-1239] |
| 31 | ACAUGUGUAAAGGUUUCAU | AUGAAACCUUUACACAUGU | | [948-966] | [1636-1654] |
| 32 | CUCACCAGUGCUUCAUAUA | UAUAUGAAGCACUGGUGAG | | [875-893] | [1563-1581] |
| 33 | GGAAUUUCUGGUGACAUAG | CUAUGUCACCAGAAAUUCC | | [854-872] | [1542-1560] |
| 34 | UGGGAAUUUCUGGUGACAU | AUGUCACCAGAAAUUCCCA | | [852-870] | [1540-1558] |
| 35 | ACAAUCAUCGGGUUAAAAA | UUUUUAACCCGAUGAUUGU | | [813-831] | [1501-1519] |
| 36 | GCCUUAUAACCUGCCUUUG | CAAAGGCAGGUUAUAAGGC | | [499-517] | [1187-1205] |
| 37 | UGCGUUAUCUGGGUCUGGA | UCCAGACCCAGAUAACGCA | | [178-196] | [866-884] |
| 38 | CCCUCUUGUAAGUCAUCUA | UAGAUGACUUACAAGAGGG | | [1004-1022] | [1692-1710] |
| 39 | ACCAGUGCUUCAUAUACCA | UGGUAUAUGAAGCACUGGU | | [878-896] | [1566-1584] |
| 40 | CUGGGAAUUUCUGGUGACA | UGUCACCAGAAAUUCCCAG | | [851-869] | [1539-1557] |
| 41 | CAAUCAUCGGGUUAAAAAA | UUUUUUAACCCGAUGAUUG | | [814-832] | [1502-1520] |
| 42 | UGAUAACAAUUCUGGGCAC | GUGCCCAGAAUUGUUAUCA | | [543-561] | [1231-1249] |
| 43 | UCGCAUGCUGAUAACAAUU | AAUUGUUAUCAGCAUGCGA | | [535-553] | [1223-1241] |
| 44 | GCCUCGCAUGCUGAUAACA | UGUUAUCAGCAUGCGAGGC | | [532-550] | [1220-1238] |
| 45 | GAAUUUCUGGUGACAUAGA | UCUAUGUCACCAGAAAUUC | | [855-873] | [1543-1561] |
| 46 | UCAUCGGGUUAAAAAACUC | GAGUUUUUUAACCCGAUGA | | [817-835] | [1505-1523] |
| 47 | AGUACAAUCAUCGGGUUAA | UUAACCCGAUGAUUGUACU | | [810-828] | [1498-1516] |
| 48 | AGGAGAGUCAUUGUUUGCA | UGCAAACAAUGACUCUCCU | | [653-671] | [1341-1359] |
| 49 | CAGUGCUUCAUAUACCAUG | CAUGGUAUAUGAAGCACUG | | [880-898] | [1568-1586] |
| 50 | CAGUACAAUCAUCGGGUUA | UAACCCGAUGAUUGUACUG | | [809-827] | [1497-1515] |
| 51 | CAAUUCUGGGCACGGUGAA | UUCACCGUGCCCAGAAUUG | | [549-567] | [1237-1255] |
| 52 | GUAAAGGUUUCAUGUUCAC | GUGAACAUGAAACCUUUAC | | [954-972] | [1642-1660] |
| 53 | GUCAUUGUUUGCAAUACAA | UUGUAUUGCAAACAAUGAC | | [659-677] | [1347-1365] |
| 54 | CUGAUAACAAUUCUGGGCA | UGCCCAGAAUUGUUAUCAG | | [542-560] | [1230-1248] |
| 55 | GGUUUCAUGUUCACUGUGA | UCACAGUGAACAUGAAACC | | [959-977] | [1647-1665] |
| 56 | UGCAGUACAAUCAUCGGGU | ACCCGAUGAUUGUACUGCA | | [807-825] | [1495-1513] |
| 57 | UGCAAUACAAAGCUGGAUA | UAUCCAGCUUUGUAUUGCA | | [668-686] | [1356-1374] |
| 58 | CACGCUUCAAUGAGAACAA | UUGUUCUCAUUGAAGCGUG | | [633-651] | [1321-1339] |
| 59 | UGUAAAGGUUUCAUGUUCA | UGAACAUGAAACCUUUACA | | [953-971] | [1641-1659] |
| 60 | UGUGUAAAGGUUUCAUGUU | AACAUGAAACCUUUACACA | | [951-969] | [1639-1657] |
| 61 | GGGAAGGGAAGAAAGACAG | CUGUCUUUCUUCCCUUCCC | Rat | [694-712] | [1382-1400] |
| 62 | UUGCAAUACAAAGCUGGAU | AUCCAGCUUUGUAUUGCAA | | [667-685] | [1355-1373] |
| 63 | AGAAUUGCUUUAGAUUUCC | GGAAAUCUAAAGCAAUUCU | | [581-599] | [1269-1287] |
| 64 | GCUUCAUAUACCAUGAUAU | AUAUCAUGGUAUAUGAAGC | | [884-902] | [1572-1590] |
| 65 | UGCUUCAUAUACCAUGAUA | UAUCAUGGUAUAUGAAGCA | | [883-901] | [1571-1589] |
| 66 | CUUCAAGGUUGCAGUGAAU | AUUCACUGCAACCUUGAAG | | [775-793] | [1463-1481] |
| 67 | UCAAUGAGAACAACAGGAG | CUCCUGUUGUUCUCAUUGA | Ms | [639-657] | [1327-1345] |
| 68 | CCACGCUUCAAUGAGAACA | UGUUCUCAUUGAAGCGUGG | | [632-650] | [1320-1338] |
| 69 | UCCCUCUUGUAAGUCAUCU | AGAUGACUUACAAGAGGGA | | [1003-1021] | [1691-1709] |
| 70 | ACUUCAAGGUUGCAGUGAA | UUCACUGCAACCUUGAAGU | | [774-792] | [1462-1480] |
| 71 | ACAGUCGGUUUUCCCAUUU | AAAUGGGAAAACCGACUGU | | [709-727] | [1397-1415] |
| 72 | CGCUUCAAUGAGAACAACA | UGUUGUUCUCAUUGAAGCG | Rat, Ms | [635-653] | [1323-1341] |
| 73 | ACGCUUCAAUGAGAACAAC | GUUGUUCUCAUUGAAGCGU | | [634-652] | [1322-1340] |
| 74 | GGAAUGAUGUUGCCUUCCA | UGGAAGGCAACAUCAUUCC | Ms | [606-624] | [1294-1312] |
| 75 | UCUUGUAAGUCAUCUACUU | AAGUAGAUGACUUACAAGA | | [1007-1025] | [1695-1713] |
| 76 | AGUCAUUGUUUGCAAUACA | UGUAUUGCAAACAAUGACU | | [658-676] | [1346-1364] |
| 77 | GCUUCAAUGAGAACAACAG | CUGUUGUUCUCAUUGAAGC | Rat, Ms | [636-654] | [1324-1342] |
| 78 | ACCCACGCUUCAAUGAGAA | UUCUCAUUGAAGCGUGGGU | | [630-648] | [1318-1336] |
| 79 | GAGGGAAUGAUGUUGCCUU | AAGGCAACAUCAUUCCCUC | Ms | [603-621] | [1291-1309] |
| 80 | UGAAGCCCAAUGCAAACAG | CUGUUUGCAUUGGGCUUCA | | [564-582] | [1252-1270] |
| 81 | CUCAAUGAAAUCAGCAAAC | GUUUGCUGAUUUCAUUGAG | | [833-851] | [1521-1539] |
| 82 | CCCAUUUGAAAGUGGGAAA | UUUCCCACUUUCAAAUGGG | | [721-739] | [1409-1427] |
| 83 | ACGGUGAAGCCCAAUGCAA | UUGCAUUGGGCUUCACCGU | | [560-578] | [1248-1266] |
| 84 | GCCACUGAUUGUGCCUUAU | AUAAGGCACAAUCAGUGGC | | [487-505] | [1175-1193] |
| 85 | CCUCUUGUAAGUCAUCUAC | GUAGAUGACUUACAAGAGG | | [1005-1023] | [1693-1711] |
| 86 | CUAAACCUUACAUGUGUAA | UUACACAUGUAAGGUUUAG | | [939-957] | [1627-1645] |
| 87 | GUGCUUCAUAUACCAUGAU | AUCAUGGUAUAUGAAGCAC | | [882-900] | [1570-1588] |
| 88 | CAGUCGGUUUUCCCAUUUG | CAAAUGGGAAAACCGACUG | | [710-728] | [1398-1416] |
| 89 | GGAAACCCAAACCCUCAAG | CUUGAGGGUUUGGGUUUCC | Ms | [194-212] | [882-900] |
| 90 | CUCACUUGUUGCAGUACAA | UUGUACUGCAACAAGUGAG | | [798-816] | [1486-1504] |
| 91 | AGAGUCAUUGUUUGCAAUA | UAUUGCAAACAAUGACUCU | | [656-674] | [1344-1362] |
| 92 | AUGUUGCCUUCCACUUUAA | UUAAAGUGGAAGGCAACAU | Ms | [612-630] | [1300-1318] |
| 93 | UCGGGUUAAAAAACUCAAU | AUUGAGUUUUUUAACCCGA | | [820-838] | [1508-1526] |
| 94 | CCACUUCAAGGUUGCAGUG | CACUGCAACCUUGAAGUGG | | [772-790] | [1460-1478] |
| 95 | CCAUUUGAAAGUGGGAAAC | GUUUCCCACUUUCAAAUGG | | [722-740] | [1410-1428] |
| 96 | GGUUUUCCCAUUUGAAAGU | ACUUUCAAAUGGGAAAACC | | [715-733] | [1403-1421] |
| 97 | GUCGGUUUUCCCAUUUGAA | UUCAAAUGGGAAAACCGAC | | [712-730] | [1400-1418] |
| 98 | GAGUCAUUGUUUGCAAUAC | GUAUUGCAAACAAUGACUC | | [657-675] | [1345-1363] |
| 99 | GAAUGAUGUUGCCUUCCAC | GUGGAAGGCAACAUCAUUC | Ms | [607-625] | [1295-1313] |
| 100 | GCUUUAGAUUUCCAAAGAG | CUCUUUGGAAAUCUAAAGC | | [587-605] | [1275-1293] |
| 101 | UGCUUUAGAUUUCCAAAGA | UCUUUGGAAAUCUAAAGCA | | [586-604] | [1274-1292] |
| 102 | CUCGCAUGCUGAUAACAAU | AUUGUUAUCAGCAUGCGAG | | [534-552] | [1222-1240] |
| 103 | CCAUCUUCUGGACAGCCAA | UUGGCUGUCCAGAAGAUGG | | [419-437] | [1107-1125] |
| 104 | UGGAAACCCAAACCCUCAA | UUGAGGGUUUGGGUUUCCA | Ms | [193-211] | [881-899] |
| 105 | GAAAUCAGCAAACUGGGAA | UUCCCAGUUUGCUGAUUUC | | [839-857] | [1527-1545] |
| 106 | GCUCACUUGUUGCAGUACA | UGUACUGCAACAAGUGAGC | | [797-815] | [1485-1503] |
| 107 | CAGUGAAUGAUGCUCACUU | AAGUGAGCAUCAUUCACUG | | [786-804] | [1474-1492] |
| 108 | CCCACGCUUCAAUGAGAAC | GUUCUCAUUGAAGCGUGGG | | [631-649] | [1319-1337] |
| 109 | AGAUUUCCAAAGAGGGAAU | AUUCCCUCUUUGGAAAUCU | | [592-610] | [1280-1298] |
| 110 | GCAAACAGAAUUGCUUUAG | CUAAAGCAAUUCUGUUUGC | | [575-593] | [1263-1281] |
| 111 | CCCAAUGCAAACAGAAUUG | CAAUUCUGUUUGCAUUGGG | | [569-587] | [1257-1275] |
| 112 | CCUUACAUGUGUAAAGGUU | AACCUUUACACAUGUAAGG | | [944-962] | [1632-1650] |
| 113 | AGUCGGUUUUCCCAUUUGA | UCAAAUGGGAAAACCGACU | | [711-729] | [1399-1417] |
| 114 | CGCAUGCUGAUAACAAUUC | GAAUUGUUAUCAGCAUGCG | | [536-554] | [1224-1242] |
| 115 | UGCCUCGCAUGCUGAUAAC | GUUAUCAGCAUGCGAGGCA | | [531-549] | [1219-1237] |
| 116 | CUACCCAUCUUCUGGACAG | CUGUCCAGAAGAUGGGUAG | | [415-433] | [1103-1121] |
| 117 | CUGGUGACAUAGACCUCAC | GUGAGGUCUAUGUCACCAG | | [861-879] | [1549-1567] |
| 118 | GCAGUGAAUGAUGCUCACU | AGUGAGCAUCAUUCACUGC | | [785-803] | [1473-1491] |
| 119 | ACCACUUCAAGGUUGCAGU | ACUGCAACCUUGAAGUGGU | | [771-789] | [1459-1477] |
| 120 | UGCAAACAGAAUUGCUUUA | UAAAGCAAUUCUGUUUGCA | | [574-592] | [1262-1280] |
| 121 | AAAUCAGCAAACUGGGAAU | AUUCCCAGUUUGCUGAUUU | | [840-858] | [1528-1546] |
| 122 | UGCAGUGAAUGAUGCUCAC | GUGAGCAUCAUUCACUGCA | | [784-802] | [1472-1490] |
| 123 | CCUGACCACUUCAAGGUUG | CAACCUUGAAGUGGUCAGG | | [767-785] | [1455-1473] |
| 124 | UCCCAUUUGAAAGUGGGAA | UUCCCACUUUCAAAUGGGA | | [720-738] | [1408-1426] |
| 125 | AGACAGUCGGUUUUCCCAU | AUGGGAAAACCGACUGUCU | | [707-725] | [1395-1413] |
| 126 | CAACAGGAGAGUCAUUGUU | AACAAUGACUCUCCUGUUG | | [649-667] | [1337-1355] |
| 127 | AGGGAAUGAUGUUGCCUUC | GAAGGCAACAUCAUUCCCU | Ms | [604-622] | [1292-1310] |
| 128 | CCAAUGCAAACAGAAUUGC | GCAAUUCUGUUUGCAUUGG | | [570-588] | [1258-1276] |
| 129 | AGCCCAAUGCAAACAGAAU | AUUCUGUUUGCAUUGGGCU | | [567-585] | [1255-1273] |
| 130 | UGUUCACUGUGAGUGAAAA | UUUUCACUCACAGUGAACA | | [966-984] | [1654-1672] |
| 131 | UGAUGCGUUAUCUGGGUCU | AGACCCAGAUAACGCAUCA | | [175-193] | [863-881] |
| 132 | AAACCUUACAUGUGUAAAG | CUUUACACAUGUAAGGUUU | | [941-959] | [1629-1647] |
| 133 | GUGACAUAGACCUCACCAG | CUGGUGAGGUCUAUGUCAC | | [864-882] | [1552-1570] |
| 134 | UCAGCAAACUGGGAAUUUC | GAAAUUCCCAGUUUGCUGA | | [843-861] | [1531-1549] |
| 135 | GGUUGAACCUGACCACUUC | GAAGUGGUCAGGUUCAACC | | [760-778] | [1448-1466] |
| 136 | UGGUUGAACCUGACCACUU | AAGUGGUCAGGUUCAACCA | | [759-777] | [1447-1465] |
| 137 | GUGGGAAACCAUUCAAAAU | AUUUUGAAUGGUUUCCCAC | | [732-750] | [1420-1438] |
| 138 | UCGGUUUUCCCAUUUGAAA | UUUCAAAUGGGAAAACCGA | | [713-731] | [1401-1419] |
| 139 | UGGGUCUGGAAACCCAAAC | GUUUGGGUUUCCAGACCCA | | [187-205] | [875-893] |
| 140 | CAGUGAAUUACCUGUCUCA | UGAGACAGGUAAUUCACUG | | [1037-1055] | [1725-1743] |
| 141 | ACAGUGAAUUACCUGUCUC | GAGACAGGUAAUUCACUGU | | [1036-1054] | [1724-1742] |
| 142 | AUCGGGUUAAAAAACUCAA | UUGAGUUUUUUAACCCGAU | | [819-837] | [1507-1525] |
| 143 | AUAGACCUCACCAGUGCUU | AAGCACUGGUGAGGUCUAU | | [869-887] | [1557-1575] |
| 144 | AGUGAAUGAUGCUCACUUG | CAAGUGAGCAUCAUUCACU | | [787-805] | [1475-1493] |
| 145 | ACCUGACCACUUCAAGGUU | AACCUUGAAGUGGUCAGGU | | [766-784] | [1454-1472] |
| 146 | UUGAACCUGACCACUUCAA | UUGAAGUGGUCAGGUUCAA | | [762-780] | [1450-1468] |
| 147 | CGGUUUUCCCAUUUGAAAG | CUUUCAAAUGGGAAAACCG | | [714-732] | [1402-1420] |
| 148 | AGAACAACAGGAGAGUCAU | AUGACUCUCCUGUUGUUCU | Ms | [645-663] | [1333-1351] |
| 149 | UCACUUGUUGCAGUACAAU | AUUGUACUGCAACAAGUGA | | [799-817] | [1487-1505] |
| 150 | AACUCAAUGAAAUCAGCAA | UUGCUGAUUUCAUUGAGUU | | [831-849] | [1519-1537] |
| 151 | AUGCUCACUUGUUGCAGUA | UACUGCAACAAGUGAGCAU | | [795-813] | [1483-1501] |
| 152 | AAAGUGGGAAACCAUUCAA | UUGAAUGGUUUCCCACUUU | | [729-747] | [1417-1435] |
| 153 | CAUGUUCACUGUGAGUGAA | UUCACUCACAGUGAACAUG | | [964-982] | [1652-1670] |
| 154 | AAGUGGGAAACCAUUCAAA | UUUGAAUGGUUUCCCACUU | | [730-748] | [1418-1436] |
| 155 | CACUGUGAGUGAAAAUUUU | AAAAUUUUCACUCACAGUG | | [970-988] | [1658-1676] |
| 156 | UCACUGUGAGUGAAAAUUU | AAAUUUUCACUCACAGUGA | | [969-987] | [1657-1675] |
| 157 | ACUCAAUGAAAUCAGCAAA | UUUGCUGAUUUCAUUGAGU | | [832-850] | [1520-1538] |
| 158 | UUCACUGUGAGUGAAAAUU | AAUUUUCACUCACAGUGAA | | [968-986] | [1656-1674] |
| 159 | GCAGACGGCUUCUCACUUA | UAAGUGAGAAGCCGUCUGC | | | |
| 160 | AGCGGAAAAUGGCAGACAA | UUGUCUGCCAUUUUCCGCU | | | |

TABLE B12
LGALS3-19-mer oligomer pairs lectin, galactoside-binding

| | | | | Human- | |
|---|---|---|---|---|---|
| | | | Other | 115430222: | Human- |
| No. | Sense | AntiSense | Sp | 152-904 | 115430224: — |

| | | | | | |
|---|---|---|---|---|---|
| 1 | CGGUGAAGCCCAAUGCAAA | UUUGCAUUGGGCUUCACCG | | [561-579] | [1249-1267] |
| 2 | GGAGAGUCAUUGUUUGCAA | UUGCAAACAAUGACUCUCC | | [654-672] | [1342-1360] |
| 3 | GCAAUACAAAGCUGGAUAA | UUAUCCAGCUUUGUAUUGC | | [669-687] | [1357-1375] |
| 4 | AUACAAAGCUGGAUAAUAA | UUAUUAUCCAGCUUUGUAU | | [672-690] | [1360-1378] |
| 5 | CAAUGAGAACAACAGGAGA | UCUCCUGUUGUUCUCAUUG | Ms | [640-658] | [1328-1346] |
| 6 | AGUGGGAAACCAUUCAAAA | UUUUGAAUGGUUUCCCACU | | [731-749] | [1419-1437] |
| 7 | GGAAACCAUUCAAAAUACA | UGUAUUUUGAAUGGUUUCC | | [735-753] | [1423-1441] |
| 8 | CAUCAAUAUCCCUCUUGUA | UACAAGAGGGAUAUUGAUG | | [995-1013] | [1683-1701] |
| 9 | GGUUAAAAAACUCAAUGAA | UUCAUUGAGUUUUUUAACC | | [823-841] | [1511-1529] |
| 10 | GUACAAUCAUCGGGUUAAA | UUUAACCCGAUGAUUGUAC | | [811-829] | [1499-1517] |
| 11 | GAAACCAUUCAAAAUACAA | UUGUAUUUUGAAUGGUUUC | | [736-754] | [1424-1442] |
| 12 | AAGUCAUCUACUUAAUAAA | UUUAUUAAGUAGAUGACUU | | [1013-1031] | [1701-1719] |
| 13 | CACUGAUUGUGCCUUAUAA | UUAUAAGGCACAAUCAGUG | | [489-507] | [1177-1195] |
| 14 | CAUUCAAAAUACAAGUACU | AGUACUUGUAUUUUGAAUG | | [741-759] | [1429-1447] |
| 15 | GUUAAAAAACUCAAUGAAA | UUUCAUUGAGUUUUUUAAC | | [824-842] | [1512-1530] |
| 16 | GAAGCCCAAUGCAAACAGA | UCUGUUUGCAUUGGGCUUC | | [565-583] | [1253-1271] |
| 17 | GGAAGGGAAGAAAGACAGU | ACUGUCUUUCUUCCCUUCC | Rat | [695-713] | [1383-1401] |
| 18 | GAAUCUAAACCUUACAUGU | ACAUGUAAGGUUUAGAUUC | | [935-953] | [1623-1641] |
| 19 | GAGUGAAAAUUUUUACAUU | AAUGUAAAAAUUUUCACUC | | [976-994] | [1664-1682] |
| 20 | GAUGUUGCCUUCCACUUUA | UAAAGUGGAAGGCAACAUC | Ms | [611-629] | [1299-1317] |
| 21 | ACCAUGAUAUAAUCUGAAA | UUUCAGAUUAUAUCAUGGU | | [893-911] | [1581-1599] |
| 22 | CCUCACCAGUGCUUCAUAU | AUAUGAAGCACUGGUGAGG | | [874-892] | [1562-1580] |
| 23 | CUGUGAGUGAAAAUUUUUA | UAAAAAUUUUCACUCACAG | | [972-990] | [1660-1678] |
| 24 | GUGCCUCGCAUGCUGAUAA | UUAUCAGCAUGCGAGGCAC | | [530-548] | [1218-1236] |
| 25 | CUGGGUCUGGAAACCCAAA | UUUGGGUUUCCAGACCCAG | | [186-204] | [874-892] |
| 26 | GAAGAAAGACAGUCGGUUU | AAACCGACUGUCUUUCUUC | | [701-719] | [1389-1407] |
| 27 | CUUCAUAUACCAUGAUAUA | UAUAUCAUGGUAUAUGAAG | | [885-903] | [1573-1591] |
| 28 | AAAUACAAGUACUGGUUGA | UCAACCAGUACUUGUAUUU | | [747-765] | [1435-1453] |
| 29 | UCAUUGUUUGCAAUACAAA | UUUGUAUUGCAAACAAUGA | | [660-678] | [1348-1366] |
| 30 | GGUGAAGCCCAAUGCAAAC | GUUUGCAUUGGGCUUCACC | | [562-580] | [1250-1268] |
| 31 | AUACCAUGAUAUAAUCUGA | UCAGAUUAUAUCAUGGUAU | | [891-909] | [1579-1597] |
| 32 | CAAACAGAAUUGCUUUAGA | UCUAAAGCAAUUCUGUUUG | | [576-594] | [1264-1282] |
| 33 | GAAAGUGGGAAACCAUUCA | UGAAUGGUUUCCCACUUUC | | [728-746] | [1416-1434] |
| 34 | AAUACAAAGCUGGAUAAUA | UAUUAUCCAGCUUUGUAUU | | [671-689] | [1359-1377] |
| 35 | GCAUGCUGAUAACAAUUCU | AGAAUUGUUAUCAGCAUGC | | [537-555] | [1225-1243] |
| 36 | AAGCCCAAUGCAAACAGAA | UUCUGUUUGCAUUGGGCUU | | [566-584] | [1254-1272] |
| 37 | CACUUUAACCCACGCUUCA | UGAAGCGUGGGUUAAAGUG | | [623-641] | [1311-1329] |
| 38 | CCAAAGAGGGAAUGAUGUU | AACAUCAUUCCCUCUUUGG | | [598-616] | [1286-1304] |
| 39 | UCUGGGUCUGGAAACCCAA | UUGGGUUUCCAGACCCAGA | | [185-203] | [873-891] |
| 40 | CAUUGUUUGCAAUACAAAG | CUUUGUAUUGCAAACAAUG | | [661-679] | [1349-1367] |
| 41 | AGUGAAUUACCUGUCUCAA | UUGAGACAGGUAAUUCACU | | [1038-1056] | [1726-1744] |
| 42 | UCAAGGUUGCAGUGAAUGA | UCAUUCACUGCAACCUUGA | | [777-795] | [1465-1483] |
| 43 | GGGAAACCAUUCAAAAUAC | GUAUUUUGAAUGGUUUCCC | | [734-752] | [1422-1440] |
| 44 | AAGUACUGGUUGAACCUGA | UCAGGUUCAACCAGUACUU | | [753-771] | [1441-1459] |
| 45 | UGGAGCACCUGGAGCUUAU | AUAAGCUCCAGGUGCUCCA | | [343-361] | [1031-1049] |
| 46 | CAUUCAUCAAUAUCCCUCU | AGAGGGAUAUUGAUGAAUG | | [991-1009] | [1679-1697] |
| 47 | AGUGCUUCAUAUACCAUGA | UCAUGGUAUAUGAAGCACU | | [881-899] | [1569-1587] |
| 48 | AAUACAAGUACUGGUUGAA | UUCAACCAGUACUUGUAUU | | [748-766] | [1436-1454] |
| 49 | GUGAAAAUUUUUACAUUCA | UGAAUGUAAAAAUUUUCAC | | [978-996] | [1666-1684] |
| 50 | UACCAUGAUAUAAUCUGAA | UUCAGAUUAUAUCAUGGUA | | [892-910] | [1580-1598] |
| 51 | CUGGAAACCCAAACCCUCA | UGAGGGUUUGGGUUUCCAG | Ms | [192-210] | [880-898] |
| 52 | UGUAAGUCAUCUACUUAAU | AUUAAGUAGAUGACUUACA | | [1010-1028] | [1698-1716] |
| 53 | GUGAGUGAAAAUUUUUACA | UGUAAAAAUUUUCACUCAC | | [974-992] | [1662-1680] |
| 54 | UCAUAUACCAUGAUAUAAU | AUUAUAUCAUGGUAUAUGA | | [887-905] | [1575-1593] |
| 55 | ACUUUAACCCACGCUUCAA | UUGAAGCGUGGGUUAAAGU | | [624-642] | [1312-1330] |
| 56 | CAGCAAACUGGGAAUUUCU | AGAAAUUCCCAGUUUGCUG | | [844-862] | [1532-1550] |
| 57 | GAAUUGCUUUAGAUUUCCA | UGGAAAUCUAAAGCAAUUC | | [582-600] | [1270-1288] |
| 58 | GCCCAAUGCAAACAGAAUU | AAUUCUGUUUGCAUUGGGC | | [568-586] | [1256-1274] |
| 59 | AGUCAUCUACUUAAUAAAU | AUUUAUUAAGUAGAUGACU | | [1014-1032] | [1702-1720] |
| 60 | CAUCGGGUUAAAAAACUCA | UGAGUUUUUUAACCCGAUG | | [818-836] | [1506-1524] |
| 61 | UUUGCAAUACAAAGCUGGA | UCCAGCUUUGUAUUGCAAA | | [666-684] | [1354-1372] |
| 62 | GAUUGUGCCUUAUAACCUG | CAGGUUAUAAGGCACAAUC | | [493-511] | [1181-1199] |
| 63 | CAAUAUCCCUCUUGUAAGU | ACUUACAAGAGGGAUAUUG | | [998-1016] | [1686-1704] |
| 64 | UGACAUAGACCUCACCAGU | ACUGGUGAGGUCUAUGUCA | | [865-883] | [1553-1571] |
| 65 | CAUGAUGCGUUAUCUGGGU | ACCCAGAUAACGCAUCAUG | | [173-191] | [861-879] |
| 66 | AGUACUGGUUGAACCUGAC | GUCAGGUUCAACCAGUACU | | [754-772] | [1442-1460] |
| 67 | GUAAGUCAUCUACUUAAUA | UAUUAAGUAGAUGACUUAC | | [1011-1029] | [1699-1717] |
| 68 | CAAACUGGGAAUUUCUGGU | ACCAGAAAUUCCCAGUUUG | | [847-865] | [1535-1553] |
| 69 | GACAGUCGGUUUUCCCAUU | AAUGGGAAAACCGACUGUC | | [708-726] | [1396-1414] |
| 70 | AGACCUCACCAGUGCUUCA | UGAAGCACUGGUGAGGUCU | | [871-889] | [1559-1577] |
| 71 | UGAAAUCAGCAAACUGGGA | UCCCAGUUUGCUGAUUUCA | | [838-856] | [1526-1544] |
| 72 | AAAGCUGGAUAAUAACUGG | CCAGUUAUUAUCCAGCUUU | | [676-694] | [1364-1382] |
| 73 | AUCAAUAUCCCUCUUGUAA | UUACAAGAGGGAUAUUGAU | | [996-1014] | [1684-1702] |
| 74 | GGCCACUGAUUGUGCCUUA | UAAGGCACAAUCAGUGGCC | | [486-504] | [1174-1192] |
| 75 | AACCCACGCUUCAAUGAGA | UCUCAUUGAAGCGUGGGUU | | [629-647] | [1317-1335] |
| 76 | CAUAUACCAUGAUAUAAUC | GAUUAUAUCAUGGUAUAUG | | [888-906] | [1576-1594] |
| 77 | UACAAAGCUGGAUAAUAAC | GUUAUUAUCCAGCUUUGUA | | [673-691] | [1361-1379] |
| 78 | GAAAAUUUUUACAUUCAUC | GAUGAAUGUAAAAAUUUUC | | [980-998] | [1668-1686] |
| 79 | AACUGGGAAUUUCUGGUGA | UCACCAGAAAUUCCCAGUU | | [849-867] | [1537-1555] |
| 80 | GGUGACAUAGACCUCACCA | UGGUGAGGUCUAUGUCACC | | [863-881] | [1551-1569] |
| 81 | ACAGGAGAGUCAUUGUUUG | CAAACAAUGACUCUCCUGU | | [651-669] | [1339-1357] |
| 82 | UAUCCCUCUUGUAAGUCAU | AUGACUUACAAGAGGGAUA | | [1001-1019] | [1689-1707] |
| 83 | UCAUCAAUAUCCCUCUUGU | ACAAGAGGGAUAUUGAUGA | | [994-1012] | [1682-1700] |
| 84 | AUUGCUUUAGAUUUCCAAA | UUUGGAAAUCUAAAGCAAU | | [584-602] | [1272-1290] |
| 85 | ACAAUUCUGGGCACGGUGA | UCACCGUGCCCAGAAUUGU | | [548-566] | [1236-1254] |
| 86 | GAACAACAGGAGAGUCAUU | AAUGACUCUCCUGUUGUUC | Ms | [646-664] | [1334-1352] |
| 87 | GUCAUCUACUUAAUAAAUA | UAUUUAUUAAGUAGAUGAC | | [1015-1033] | [1703-1721] |
| 88 | CUUACAUGUGUAAAGGUUU | AAACCUUUACACAUGUAAG | | [945-963] | [1633-1651] |
| 89 | AUCUAAACCUUACAUGUGU | ACACAUGUAAGGUUUAGAU | | [937-955] | [1625-1643] |
| 90 | UACAAUCAUCGGGUUAAAA | UUUUAACCCGAUGAUUGUA | | [812-830] | [1500-1518] |
| 91 | GAUGCUCACUUGUUGCAGU | ACUGCAACAAGUGAGCAUC | | [794-812] | [1482-1500] |
| 92 | UUGAAAGUGGGAAACCAUU | AAUGGUUUCCCACUUUCAA | | [726-744] | [1414-1432] |
| 93 | CAUGUGUAAAGGUUUCAUG | CAUGAAACCUUUACACAUG | | [949-967] | [1637-1655] |
| 94 | AAAAUGGCAGACAAUUUUU | AAAAAUUGUCUGCCAUUUU | | [149-167] | [184-202] |
| 95 | AAACCAUUCAAAAUACAAG | CUUGUAUUUUGAAUGGUUU | Ms | [737-755] | [1425-1443] |
| 96 | UUGUAAGUCAUCUACUUAA | UUAAGUAGAUGACUUACAA | | [1009-1027] | [1697-1715] |
| 97 | AAUUGCUUUAGAUUUCCAA | UUGGAAAUCUAAAGCAAUU | | [583-601] | [1271-1289] |
| 98 | UCUGGUGACAUAGACCUCA | UGAGGUCUAUGUCACCAGA | | [860-878] | [1548-1566] |
| 99 | AACCAUUCAAAAUACAAGU | ACUUGUAUUUUGAAUGGUU | Ms | [738-756] | [1426-1444] |
| 100 | UAAGUCAUCUACUUAAUAA | UUAUUAAGUAGAUGACUUA | | [1012-1030] | [1700-1718] |
| 101 | AAUUUCUGGUGACAUAGAC | GUCUAUGUCACCAGAAAUU | | [856-874] | [1544-1562] |
| 102 | CCACUGAUUGUGCCUUAUA | UAUAAGGCACAAUCAGUGG | | [488-506] | [1176-1194] |
| 103 | ACUUGUUGCAGUACAAUCA | UGAUUGUACUGCAACAAGU | | [801-819] | [1489-1507] |
| 104 | UGACCACUUCAAGGUUGCA | UGCAACCUUGAAGUGGUCA | | [769-787] | [1457-1475] |
| 105 | UGGGAAACCAUUCAAAAUA | UAUUUUGAAUGGUUUCCCA | | [733-751] | [1421-1439] |
| 106 | GAAUGAUGCUCACUUGUUG | CAACAAGUGAGCAUCAUUC | | [790-808] | [1478-1496] |
| 107 | GCUCCAUGAUGCGUUAUCU | AGAUAACGCAUCAUGGAGC | | [169-187] | [857-875] |
| 108 | CUUGUAAGUCAUCUACUUA | UAAGUAGAUGACUUACAAG | | [1008-1026] | [1696-1714] |
| 109 | AAACAGAAUUGCUUUAGAU | AUCUAAAGCAAUUCUGUUU | | [577-595] | [1265-1283] |
| 110 | GGUUGCAGUGAAUGAUGCU | AGCAUCAUUCACUGCAACC | | [781-799] | [1469-1487] |
| 111 | GUUGAACCUGACCACUUCA | UGAAGUGGUCAGGUUCAAC | | [761-779] | [1449-1467] |
| 112 | AUGUGUAAAGGUUUCAUGU | ACAUGAAACCUUUACACAU | | [950-968] | [1638-1656] |
| 113 | ACUGAUUGUGCCUUAUAAC | GUUAUAAGGCACAAUCAGU | | [490-508] | [1178-1196] |
| 114 | AACAGAAUUGCUUUAGAUU | AAUCUAAAGCAAUUCUGUU | | [578-596] | [1266-1284] |
| 115 | CAAAGCUGGAUAAUAACUG | CAGUUAUUAUCCAGCUUUG | | [675-693] | [1363-1381] |
| 116 | GUUUCAUGUUCACUGUGAG | CUCACAGUGAACAUGAAAC | | [960-978] | [1648-1666] |
| 117 | ACAGAAUUGCUUUAGAUUU | AAAUCUAAAGCAAUUCUGU | | [579-597] | [1267-1285] |
| 118 | UUCAAUGAGAACAACAGGA | UCCUGUUGUUCUCAUUGAA | Ms | [638-656] | [1326-1344] |
| 119 | AAUGCAAACAGAAUUGCUU | AAGCAAUUCUGUUUGCAUU | | [572-590] | [1260-1278] |
| 120 | AAGACAGUCGGUUUUCCCA | UGGGAAAACCGACUGUCUU | | [706-724] | [1394-1412] |
| 121 | ACAAAGCUGGAUAAUAACU | AGUUAUUAUCCAGCUUUGU | | [674-692] | [1362-1380] |
| 122 | AUUCAUCAAUAUCCCUCUU | AAGAGGGAUAUUGAUGAAU | | [992-1010] | [1680-1698] |
| 123 | UAAACCUUACAUGUGUAAA | UUUACACAUGUAAGGUUUA | | [940-958] | [1628-1646] |
| 124 | UGCCUUCCACUUUAACCCA | UGGGUUAAAGUGGAAGGCA | | [616-634] | [1304-1322] |
| 125 | UGCUGAUAACAAUUCUGGG | CCCAGAAUUGUUAUCAGCA | | [540-558] | [1228-1246] |
| 126 | CAAUGCAAACAGAAUUGCU | AGCAAUUCUGUUUGCAUUG | | [571-589] | [1259-1277] |
| 127 | ACCAUUCAAAAUACAAGUA | UACUUGUAUUUUGAAUGGU | | [739-757] | [1427-1445] |
| 128 | UGAGUGAAAAUUUUUACAU | AUGUAAAAAUUUUCACUCA | | [975-993] | [1663-1681] |
| 129 | CAUGCUGAUAACAAUUCUG | CAGAAUUGUUAUCAGCAUG | | [538-556] | [1226-1244] |
| 130 | AUAUCCCUCUUGUAAGUCA | UGACUUACAAGAGGGAUAU | | [1000-1018] | [1688-1706] |
| 131 | CAUCUUCUGGACAGCCAAG | CUUGGCUGUCCAGAAGAUG | | [420-438] | [1108-1126] |
| 132 | GACAUAGACCUCACCAGUG | CACUGGUGAGGUCUAUGUC | | [866-884] | [1554-1572] |
| 133 | ACUGGGAAUUUCUGGUGAC | GUCACCAGAAAUUCCCAGU | | [850-868] | [1538-1556] |
| 134 | GCAAACUGGGAAUUUCUGG | CCAGAAAUUCCCAGUUUGC | | [846-864] | [1534-1552] |
| 135 | GAGAACAACAGGAGAGUCA | UGACUCUCCUGUUGUUCUC | Ms | [644-662] | [1332-1350] |
| 136 | AAGGUUUCAUGUUCACUGU | ACAGUGAACAUGAAACCUU | | [957-975] | [1645-1663] |
| 137 | ACUGUGAGUGAAAAUUUUU | AAAAAUUUUCACUCACAGU | | [971-989] | [1659-1677] |
| 138 | UGAUGUUGCCUUCCACUUU | AAAGUGGAAGGCAACAUCA | Ms | [610-628] | [1298-1316] |
| 139 | AUUUGAAAGUGGGAAACCA | UGGUUUCCCACUUUCAAAU | | [724-742] | [1412-1430] |
| 140 | ACAAGUACUGGUUGAACCU | AGGUUCAACCAGUACUUGU | | [751-769] | [1439-1457] |
| 141 | CAAAGAGGGAAUGAUGUUG | CAACAUCAUUCCCUCUUUG | | [599-617] | [1287-1305] |
| 142 | AGGGAAGAAAGACAGUCGG | CCGACUGUCUUUCUUCCCU | | [698-716] | [1386-1404] |
| 143 | CAUGAUAUAAUCUGAAAGG | CCUUUCAGAUUAUAUCAUG | | [895-913] | [1583-1601] |
| 144 | UUUGAAAGUGGGAAACCAU | AUGGUUUCCCACUUUCAAA | | [725-743] | [1413-1431] |
| 145 | AGCAAACUGGGAAUUUCUG | CAGAAAUUCCCAGUUUGCU | | [845-863] | [1533-1551] |
| 146 | CAUAGACCUCACCAGUGCU | AGCACUGGUGAGGUCUAUG | | [868-886] | [1556-1574] |
| 147 | UCAUGUUCACUGUGAGUGA | UCACUCACAGUGAACAUGA | | [963-981] | [1651-1669] |
| 148 | AUCUGGGUCUGGAAACCCA | UGGGUUUCCAGACCCAGAU | | [184-202] | [872-890] |
| 149 | AACAGGAGAGUCAUUGUUU | AAACAAUGACUCUCCUGUU | | [650-668] | [1338-1356] |
| 150 | UGUGCCUUAUAACCUGCCU | AGGCAGGUUAUAAGGCACA | | [496-514] | [1184-1202] |
| 151 | GUGAAGCCCAAUGCAAACA | UGUUUGCAUUGGGCUUCAC | | [563-581] | [1251-1269] |
| 152 | CCAUGAUGCGUUAUCUGGG | CCCAGAUAACGCAUCAUGG | | [172-190] | [860-878] |
| 153 | AUGCAAACAGAAUUGCUUU | AAAGCAAUUCUGUUUGCAU | | [573-591] | [1261-1279] |
| 154 | AAUGAUGUUGCCUUCCACU | AGUGGAAGGCAACAUCAUU | Ms | [608-626] | [1296-1314] |
| 155 | CCUUCCACUUUAACCCACG | CGUGGGUUAAAGUGGAAGG | | [618-636] | [1306-1324] |
| 156 | UUCAUAUACCAUGAUAUAA | UUAUAUCAUGGUAUAUGAA | | [886-904] | [1574-1592] |
| 157 | AGUGAAAAUUUUUACAUUC | GAAUGUAAAAAUUUUCACU | | [977-995] | [1665-1683] |
| 158 | CUUGUUGCAGUACAAUCAU | AUGAUUGUACUGCAACAAG | | [802-820] | [1490-1508] |
| 159 | CAGAAUUGCUUUAGAUUUC | GAAAUCUAAAGCAAUUCUG | | [580-598] | [1268-1286] |
| 160 | CAAUGAAAUCAGCAAACUG | CAGUUUGCUGAUUUCAUUG | | [835-853] | [1523-1541] |
| 161 | AAGGUUGCAGUGAAUGAUG | CAUCAUUCACUGCAACCUU | | [779-797] | [1467-1485] |
| 162 | AAACCCAAACCCUCAAGGA | UCCUUGAGGGUUUGGGUUU | Ms | [196-214] | [884-902] |
| 163 | AUAUUACAGUGAAUUACCU | AGGUAAUUCACUGUAAUAU | | [1031-1049] | [1719-1737] |
| 164 | UUAACCCACGCUUCAAUGA | UCAUUGAAGCGUGGGUUAA | | [627-645] | [1315-1333] |
| 165 | UAACAAUUCUGGGCACGGU | ACCGUGCCCAGAAUUGUUA | | [546-564] | [1234-1252] |
| 166 | GUUCACUGUGAGUGAAAAU | AUUUUCACUCACAGUGAAC | | [967-985] | [1655-1673] |
| 167 | CCUACCCAUCUUCUGGACA | UGUCCAGAAGAUGGGUAGG | | [414-432] | [1102-1120] |
| 168 | AACCCAAACCCUCAAGGAU | AUCCUUGAGGGUUUGGGUU | Ms | [197-215] | [885-903] |
| 169 | UGAUUGUGCCUUAUAACCU | AGGUUAUAAGGCACAAUCA | | [492-510] | [1180-1198] |
| 170 | UCACCAGUGCUUCAUAUAC | GUAUAUGAAGCACUGGUGA | | [876-894] | [1564-1582] |
| 171 | UGUUUGCAAUACAAAGCUG | CAGCUUUGUAUUGCAAACA | | [664-682] | [1352-1370] |
| 172 | ACAACAGGAGAGUCAUUGU | ACAAUGACUCUCCUGUUGU | Ms | [648-666] | [1336-1354] |
| 173 | AAUUCUGGGCACGGUGAAG | CUUCACCGUGCCCAGAAUU | | [550-568] | [1238-1256] |
| 174 | AUGAGAACAACAGGAGAGU | ACUCUCCUGUUGUUCUCAU | Ms | [642-660] | [1330-1348] |
| 175 | UCAAUAUCCCUCUUGUAAG | CUUACAAGAGGGAUAUUGA | | [997-1015] | [1685-1703] |
| 176 | GUGUAAAGGUUUCAUGUUC | GAACAUGAAACCUUUACAC | | [952-970] | [1640-1658] |
| 177 | GUUUGCAAUACAAAGCUGG | CCAGCUUUGUAUUGCAAAC | | [665-683] | [1353-1371] |
| 178 | CACCAGUGCUUCAUAUACC | GGUAUAUGAAGCACUGGUG | | [877-895] | [1565-1583] |
| 179 | UUAGAUUUCCAAAGAGGGA | UCCCUCUUUGGAAAUCUAA | | [590-608] | [1278-1296] |
| 180 | GAACCUGACCACUUCAAGG | CCUUGAAGUGGUCAGGUUC | | [764-782] | [1452-1470] |
| 181 | UGAAUGAUGCUCACUUGUU | AACAAGUGAGCAUCAUUCA | | [789-807] | [1477-1495] |
| 182 | CAGGAGAGUCAUUGUUUGC | GCAAACAAUGACUCUCCUG | | [652-670] | [1340-1358] |
| 183 | GUGAAUGAUGCUCACUUGU | ACAAGUGAGCAUCAUUCAC | | [788-806] | [1476-1494] |
| 184 | ACUGGUUGAACCUGACCAC | GUGGUCAGGUUCAACCAGU | | [757-775] | [1445-1463] |
| 185 | ACCUUACAUGUGUAAAGGU | ACCUUUACACAUGUAAGGU | | [943-961] | [1631-1649] |
| 186 | UACAUGUGUAAAGGUUUCA | UGAAACCUUUACACAUGUA | | [947-965] | [1635-1653] |
| 187 | UUAUAACCUGCCUUUGCCU | AGGCAAAGGCAGGUUAUAA | | [502-520] | [1190-1208] |
| 188 | UGAACCUGACCACUUCAAG | CUUGAAGUGGUCAGGUUCA | | [763-781] | [1451-1469] |
| 189 | UGUGAGUGAAAAUUUUUAC | GUAAAAAUUUUCACUCACA | | [973-991] | [1661-1679] |
| 190 | CUGGUUGAACCUGACCACU | AGUGGUCAGGUUCAACCAG | | [758-776] | [1446-1464] |
| 191 | AAUCUAAACCUUACAUGUG | CACAUGUAAGGUUUAGAUU | | [936-954] | [1624-1642] |
| 192 | UUGCAGUGAAUGAUGCUCA | UGAGCAUCAUUCACUGCAA | | [783-801] | [1471-1489] |
| 193 | CUUAAUAAAUAUUACAGUG | CACUGUAAUAUUUAUUAAG | | [1023-1041] | [1711-1729] |
| 194 | AAAAAACUCAAUGAAAUCA | UGAUUUCAUUGAGUUUUUU | | [827-845] | [1515-1533] |
| 195 | UAAAAAACUCAAUGAAAUC | GAUUUCAUUGAGUUUUUUA | | [826-844] | [1514-1532] |
| 196 | AUUCAAAAUACAAGUACUG | CAGUACUUGUAUUUUGAAU | | [742-760] | [1430-1448] |
| 197 | UCCAAAGAGGGAAUGAUGU | ACAUCAUUCCCUCUUUGGA | | [597-615] | [1285-1303] |
| 198 | UCAAUGAAAUCAGCAAACU | AGUUUGCUGAUUUCAUUGA | | [834-852] | [1522-1540] |
| 199 | AUGUUCACUGUGAGUGAAA | UUUCACUCACAGUGAACAU | | [965-983] | [1653-1671] |
| 200 | CUCCAUGAUGCGUUAUCUG | CAGAUAACGCAUCAUGGAG | | [170-188] | [858-876] |
| 201 | CCUUAUAACCUGCCUUUGC | GCAAAGGCAGGUUAUAAGG | | [500-518] | [1188-1206] |
| 202 | CUCUUGUAAGUCAUCUACU | AGUAGAUGACUUACAAGAG | | [1006-1024] | [1694-1712] |
| 203 | GAAGGGAAGAAAGACAGUC | GACUGUCUUUCUUCCCUUC | Rat | [696-714] | [1384-1402] |
| 204 | GUCUGGAAACCCAAACCCU | AGGGUUUGGGUUUCCAGAC | | [190-208] | [878-896] |
| 205 | AAAUGGCAGACAAUUUUUC | GAAAAAUUGUCUGCCAUUU | | [150-168] | [185-203] |
| 206 | AUUUCCAAAGAGGGAAUGA | UCAUUCCCUCUUUGGAAAU | | [594-612] | [1282-1300] |
| 207 | UCUUCUGGACAGCCAAGUG | CACUUGGCUGUCCAGAAGA | | [422-440] | [1110-1128] |
| 208 | UAGAUUUCCAAAGAGGGAA | UUCCCUCUUUGGAAAUCUA | | [591-609] | [1279-1297] |
| 209 | GUUAUCUGGGUCUGGAAAC | GUUUCCAGACCCAGAUAAC | | [181-199] | [869-887] |
| 210 | UACUGGUUGAACCUGACCA | UGGUCAGGUUCAACCAGUA | | [756-774] | [1444-1462] |
| 211 | AAAAUACAAGUACUGGUUG | CAACCAGUACUUGUAUUUU | | [746-764] | [1434-1452] |
| 212 | AAGGGAAGAAAGACAGUCG | CGACUGUCUUUCUUCCCUU | | [697-715] | [1385-1403] |
| 213 | CUUCAAUGAGAACAACAGG | CCUGUUGUUCUCAUUGAAG | Ms | [637-655] | [1325-1343] |
| 214 | AUACAAGUACUGGUUGAAC | GUUCAACCAGUACUUGUAU | | [749-767] | [1437-1455] |
| 215 | AAACUCAAUGAAAUCAGCA | UGCUGAUUUCAUUGAGUUU | | [830-848] | [1518-1536] |
| 216 | CUGACCACUUCAAGGUUGC | GCAACCUUGAAGUGGUCAG | Ms | [768-786] | [1456-1474] |
| 217 | AAUCAUCGGGUUAAAAAAC | GUUUUUUAACCCGAUGAUU | | [815-833] | [1503-1521] |
| 218 | GAUGCGUUAUCUGGGUCUG | CAGACCCAGAUAACGCAUC | | [176-194] | [864-882] |
| 219 | AAAACUCAAUGAAAUCAGC | GCUGAUUUCAUUGAGUUUU | | [829-847] | [1517-1535] |
| 220 | GUUGCAGUACAAUCAUCGG | CCGAUGAUUGUACUGCAAC | | [805-823] | [1493-1511] |
| 221 | AAAUAUUACAGUGAAUUAC | GUAAUUCACUGUAAUAUUU | | [1029-1047] | [1717-1735] |
| 222 | CUUUAGAUUUCCAAAGAGG | CCUCUUUGGAAAUCUAAAG | | [588-606] | [1276-1294] |
| 223 | UUUCCAAAGAGGGAAUGAU | AUCAUUCCCUCUUUGGAAA | | [595-613] | [1283-1301] |
| 224 | CACUUCAAGGUUGCAGUGA | UCACUGCAACCUUGAAGUG | | [773-791] | [1461-1479] |
| 225 | AAAAACUCAAUGAAAUCAG | CUGAUUUCAUUGAGUUUUU | | [828-846] | [1516-1534] |
| 226 | AAUGAGAACAACAGGAGAG | CUCUCCUGUUGUUCUCAUU | Ms | [641-659] | [1329-1347] |
| 227 | CUGAUUGUGCCUUAUAACC | GGUUAUAAGGCACAAUCAG | | [491-509] | [1179-1197] |
| 228 | AACAAUUCUGGGCACGGUG | CACCGUGCCCAGAAUUGUU | | [547-565] | [1235-1253] |
| 229 | AUGAUGUUGCCUUCCACUU | AAGUGGAAGGCAACAUCAU | Ms | [609-627] | [1297-1315] |
| 230 | GAAAGACAGUCGGUUUUCC | GGAAAACCGACUGUCUUUC | | [704-722] | [1392-1410] |
| 231 | AAGCUGGAUAAUAACUGGG | CCCAGUUAUUAUCCAGCUU | | [677-695] | [1365-1383] |
| 232 | UUACAUUCAUCAAUAUCCC | GGGAUAUUGAUGAAUGUAA | | [988-1006] | [1676-1694] |
| 233 | AAUGGCAGACAAUUUUUCG | CGAAAAAUUGUCUGCCAUU | | [151-169] | [186-204] |
| 234 | AUGAAAUCAGCAAACUGGG | CCCAGUUUGCUGAUUUCAU | | [837-855] | [1525-1543] |
| 235 | UUCCCAUUUGAAAGUGGGA | UCCCACUUUCAAAUGGGAA | | [719-737] | [1407-1425] |
| 236 | UGUUGCCUUCCACUUUAAC | GUUAAAGUGGAAGGCAACA | Ms | [613-631] | [1301-1319] |
| 237 | CUUAUAACCUGCCUUUGCC | GGCAAAGGCAGGUUAUAAG | | [501-519] | [1189-1207] |
| 238 | UGGUGACAUAGACCUCACC | GGUGAGGUCUAUGUCACCA | | [862-880] | [1550-1568] |
| 239 | CUUCCACUUUAACCCACGC | GCGUGGGUUAAAGUGGAAG | | [619-637] | [1307-1325] |
| 240 | AAGAGGGAAUGAUGUUGCC | GGCAACAUCAUUCCCUCUU | | [601-619] | [1289-1307] |
| 241 | AUCAUCGGGUUAAAAAACU | AGUUUUUUAACCCGAUGAU | | [816-834] | [1504-1522] |
| 242 | AGGUUGCAGUGAAUGAUGC | GCAUCAUUCACUGCAACCU | | [780-798] | [1468-1486] |
| 243 | UCUGGAAACCCAAACCCUC | GAGGGUUUGGGUUUCCAGA | Rat, Ms | [191-209] | [879-897] |
| 244 | AAAGGUUUCAUGUUCACUG | CAGUGAACAUGAAACCUUU | | [956-974] | [1644-1662] |
| 245 | AAACCCUCAAGGAUGGCCU | AGGCCAUCCUUGAGGGUUU | | [202-220] | [890-908] |
| 246 | AUGAUAUAAUCUGAAAGGG | CCCUUUCAGAUUAUAUCAU | | [896-914] | [1584-1602] |
| 247 | UUCCAAAGAGGGAAUGAUG | CAUCAUUCCCUCUUUGGAA | | [596-614] | [1284-1302] |
| 248 | AGAGGGAAUGAUGUUGCCU | AGGCAACAUCAUUCCCUCU | Ms | [602-620] | [1290-1308] |
| 249 | UACAUUCAUCAAUAUCCCU | AGGGAUAUUGAUGAAUGUA | | [989-1007] | [1677-1695] |
| 250 | AAUCAGCAAACUGGGAAUU | AAUUCCCAGUUUGCUGAUU | | [841-859] | [1529-1547] |
| 251 | GUUGCCUUCCACUUUAACC | GGUUAAAGUGGAAGGCAAC | Ms | [614-632] | [1302-1320] |
| 252 | UGAAAGUGGGAAACCAUUC | GAAUGGUUUCCCACUUUCA | | [727-745] | [1415-1433] |
| 253 | AAAGAGGGAAUGAUGUUGC | GCAACAUCAUUCCCUCUUU | | [600-618] | [1288-1306] |
| 254 | GCUGAUAACAAUUCUGGGC | GCCCAGAAUUGUUAUCAGC | | [541-559] | [1229-1247] |
| 255 | UUUCAUGUUCACUGUGAGU | ACUCACAGUGAACAUGAAA | | [961-979] | [1649-1667] |
| 256 | AGGUUUCAUGUUCACUGUG | CACAGUGAACAUGAAACCU | | [958-976] | [1646-1664] |
| 257 | AAUAUUACAGUGAAUUACC | GGUAAUUCACUGUAAUAUU | | [1030-1048] | [1718-1736] |
| 258 | AUCAGCAAACUGGGAAUUU | AAAUUCCCAGUUUGCUGAU | | [842-860] | [1530-1548] |
| 259 | UUGUUUGCAAUACAAAGCU | AGCUUUGUAUUGCAAACAA | | [663-681] | [1351-1369] |
| 260 | UACCCAUCUUCUGGACAGC | GCUGUCCAGAAGAUGGGUA | | [416-434] | [1104-1122] |
| 261 | UUCUGGUGACAUAGACCUC | GAGGUCUAUGUCACCAGAA | | [859-877] | [1547-1565] |
| 262 | UUACAUGUGUAAAGGUUUC | GAAACCUUUACACAUGUAA | | [946-964] | [1634-1652] |
| 263 | ACAUAGACCUCACCAGUGC | GCACUGGUGAGGUCUAUGU | | [867-885] | [1555-1573] |
| 264 | ACCCAAACCCUCAAGGAUG | CAUCCUUGAGGGUUUGGGU | | [198-216] | [886-904] |
| 265 | UAAAGGUUUCAUGUUCACU | AGUGAACAUGAAACCUUUA | | [955-973] | [1643-1661] |
| 266 | UUUAACCCACGCUUCAAUG | CAUUGAAGCGUGGGUUAAA | | [626-644] | [1314-1332] |
| 267 | AACCUUACAUGUGUAAAGG | CCUUUACACAUGUAAGGUU | | [942-960] | [1630-1648] |
| 268 | CACUUGUUGCAGUACAAUC | GAUUGUACUGCAACAAGUG | | [800-818] | [1488-1506] |
| 269 | GAAACCCAAACCCUCAAGG | CCUUGAGGGUUUGGGUUUC | Ms | [195-213] | [883-901] |
| 270 | AUCUUCUGGACAGCCAAGU | ACUUGGCUGUCCAGAAGAU | | [421-439] | [1109-1127] |
| 271 | UAACCCACGCUUCAAUGAG | CUCAUUGAAGCGUGGGUUA | | [628-646] | [1316-1334] |
| 272 | GAUAACAAUUCUGGGCACG | CGUGCCCAGAAUUGUUAUC | | [544-562] | [1232-1250] |
| 273 | CAAGUACUGGUUGAACCUG | CAGGUUCAACCAGUACUUG | | [752-770] | [1440-1458] |
| 274 | UACAGUGAAUUACCUGUCU | AGACAGGUAAUUCACUGUA | | [1035-1053] | [1723-1741] |
| 275 | UUCAAAAUACAAGUACUGG | CCAGUACUUGUAUUUUGAA | | [743-761] | [1431-1449] |
| 276 | UGUUGCAGUACAAUCAUCG | CGAUGAUUGUACUGCAACA | | [804-822] | [1492-1510] |
| 277 | AAUGAAAUCAGCAAACUGG | CCAGUUUGCUGAUUUCAUU | | [836-854] | [1524-1542] |
| 278 | AACCUGACCACUUCAAGGU | ACCUUGAAGUGGUCAGGUU | | [765-783] | [1453-1471] |
| 279 | AUCCCUCUUGUAAGUCAUC | GAUGACUUACAAGAGGGAU | | [1002-1020] | [1690-1708] |
| 280 | GAUUUCCAAAGAGGGAAUG | CAUUCCCUCUUUGGAAAUC | | [593-611] | [1281-1299] |
| 281 | UACAAGUACUGGUUGAACC | GGUUCAACCAGUACUUGUA | | [750-768] | [1438-1456] |
| 282 | AUGCUGAUAACAAUUCUGG | CCAGAAUUGUUAUCAGCAU | | [539-557] | [1227-1245] |
| 283 | AAACUGGGAAUUUCUGGUG | CACCAGAAAUUCCCAGUUU | | [848-866] | [1536-1554] |
| 284 | GGGAAUGAUGUUGCCUUCC | GGAAGGCAACAUCAUUCCC | Ms | [605-623] | [1293-1311] |
| 285 | AUGCGUUAUCUGGGUCUGG | CCAGACCCAGAUAACGCAU | | [177-195] | [865-883] |
| 286 | UUUCUGGUGACAUAGACCU | AGGUCUAUGUCACCAGAAA | | [858-876] | [1546-1564] |
| 287 | UUUAGAUUUCCAAAGAGGG | CCCUCUUUGGAAAUCUAAA | | [589-607] | [1277-1295] |
| 288 | AGAAAGACAGUCGGUUUUC | GAAAACCGACUGUCUUUCU | | [703-721] | [1391-1409] |
| 289 | UGAUGCUCACUUGUUGCAG | CUGCAACAAGUGAGCAUCA | | [793-811] | [1481-1499] |
| 290 | AUUACAGUGAAUUACCUGU | ACAGGUAAUUCACUGUAAU | | [1033-1051] | [1721-1739] |
| 291 | AUUGUGCCUUAUAACCUGC | GCAGGUUAUAAGGCACAAU | | [494-512] | [1182-1200] |
| 292 | GUACUGGUUGAACCUGACC | GGUCAGGUUCAACCAGUAC | | [755-773] | [1443-1461] |
| 293 | AUUUCUGGUGACAUAGACC | GGUCUAUGUCACCAGAAAU | | [857-875] | [1545-1563] |
| 294 | AUGAUGCUCACUUGUUGCA | UGCAACAAGUGAGCAUCAU | | [792-810] | [1480-1498] |
| 295 | UUGCUUUAGAUUUCCAAAG | CUUUGGAAAUCUAAAGCAA | | [585-603] | [1273-1291] |
| 296 | GACCACUUCAAGGUUGCAG | CUGCAACCUUGAAGUGGUC | | [770-788] | [1458-1476] |
| 297 | AUAUACCAUGAUAUAAUCU | AGAUUAUAUCAUGGUAUAU | | [889-907] | [1577-1595] |
| 298 | UUAUCCUGGACAGGCACCU | AGGUGCCUGUCCAGGAUAA | | [310-328] | [998-1016] |
| 299 | GUUGCAGUGAAUGAUGCUC | GAGCAUCAUUCACUGCAAC | | [782-800] | [1470-1488] |
| 300 | AUAACAAUUCUGGGCACGG | CCGUGCCCAGAAUUGUUAU | | [545-563] | [1233-1251] |
| 301 | UGAGAACAACAGGAGAGUC | GACUCUCCUGUUGUUCUCA | Ms | [643-661] | [1331-1349] |
| 302 | AAAGACAGUCGGUUUUCCC | GGGAAAACCGACUGUCUUU | | [705-723] | [1393-1411] |
| 303 | UUCAAGGUUGCAGUGAAUG | CAUUCACUGCAACCUUGAA | | [776-794] | [1464-1482] |
| 304 | UUUACAUUCAUCAAUAUCC | GGAUAUUGAUGAAUGUAAA | | [987-1005] | [1675-1693] |
| 305 | UAUAACCUGCCUUUGCCUG | CAGGCAAAGGCAGGUUAUA | | [503-521] | [1191-1209] |
| 306 | AAUGAUGCUCACUUGUUGC | GCAACAAGUGAGCAUCAUU | | [791-809] | [1479-1497] |
| 307 | UCCAUGAUGCGUUAUCUGG | CCAGAUAACGCAUCAUGGA | | [171-189] | [859-877] |
| 308 | AUUGUUUGCAAUACAAAGC | GCUUUGUAUUGCAAACAAU | | [662-680] | [1350-1368] |
| 309 | CAUUUGAAAGUGGGAAACC | GGUUUCCCACUUUCAAAUG | | [723-741] | [1411-1429] |
| 310 | UUGCCUUCCACUUUAACCC | GGGUUAAAGUGGAAGGCAA | Ms | [615-633] | [1303-1321] |
| 311 | UUCAUCAAUAUCCCUCUUG | CAAGAGGGAUAUUGAUGAA | | [993-1011] | [1681-1699] |
| 312 | UAUACCAUGAUAUAAUCUG | CAGAUUAUAUCAUGGUAUA | | [890-908] | [1578-1596] |
| 313 | UAUUACAGUGAAUUACCUG | CAGGUAAUUCACUGUAAUA | | [1032-1050] | [1720-1738] |
| 314 | AUGAUGCGUUAUCUGGGUC | GACCCAGAUAACGCAUCAU | | [174-192] | [862-880] |
| 315 | UUCCACUUUAACCCACGCU | AGCGUGGGUUAAAGUGGAA | | [620-638] | [1308-1326] |
| 316 | UAGACCUCACCAGUGCUUC | GAAGCACUGGUGAGGUCUA | | [870-888] | [1558-1576] |
| 317 | UUGUGCCUUAUAACCUGCC | GGCAGGUUAUAAGGCACAA | | [495-513] | [1183-1201] |
| 318 | UUGUUGCAGUACAAUCAUC | GAUGAUUGUACUGCAACAA | | [803-821] | [1491-1509] |
| 319 | GUUUUCCCAUUUGAAAGUG | CACUUUCAAAUGGGAAAAC | | [716-734] | [1404-1422] |
| 320 | AUAACCUGCCUUUGCCUGG | CCAGGCAAAGGCAGGUUAU | | [504-522] | [1192-1210] |
| 321 | UUUUACAUUCAUCAAUAUC | GAUAUUGAUGAAUGUAAAA | | [986-1004] | [1674-1692] |
| 322 | UUUCCCAUUUGAAAGUGGG | CCCACUUUCAAAUGGGAAA | | [718-736] | [1406-1424] |
| 323 | UUAUCUGGGUCUGGAAACC | GGUUUCCAGACCCAGAUAA | | [182-200] | [870-888] |
| 324 | UGCUCACUUGUUGCAGUAC | GUACUGCAACAAGUGAGCA | | [796-814] | [1484-1502] |
| 325 | UUGCAGUACAAUCAUCGGG | CCCGAUGAUUGUACUGCAA | | [806-824] | [1494-1512] |
| 326 | AACAACAGGAGAGUCAUUG | CAAUGACUCUCCUGUUGUU | Ms | [647-665] | [1335-1353] |
| 327 | UUUUCCCAUUUGAAAGUGG | CCACUUUCAAAUGGGAAAA | | [717-735] | [1405-1423] |
| 328 | AAUAUCCCUCUUGUAAGUC | GACUUACAAGAGGGAUAUU | | [999-1017] | [1687-1705] |
| 329 | UUACAGUGAAUUACCUGUC | GACAGGUAAUUCACUGUAA | | [1034-1052] | [1722-1740] |
| 330 | UAUCUGGGUCUGGAAACCC | GGGUUUCCAGACCCAGAUA | | [183-201] | [871-889] |
| 331 | UUCAUGUUCACUGUGAGUG | CACUCACAGUGAACAUGAA | | [962-980] | [1650-1668] |

TABLE B13
Hi95 19-mer oligomer pairs Sestrin2 (SESN2, Hi95)

| | | | | Human-32454742: |
|---|---|---|---|---|
| No. | Sense | AntiSense | Other Sp | 354-1796 |

| | | | | |
|---|---|---|---|---|
| 1 | CGGAAUUAAUGUGCCACAA | UUGUGGCACAUUAAUUCCG | | [2103-2121] |
| 2 | GACUUCCUUUGCCCUUUUU | AAAAAGGGCAAAGGAAGUC | MK | [2373-2391] |
| 3 | CCAGUGUGCCACAUUAAAU | AUUUAAUGUGGCACACUGG | MK | [3224-3242] |
| 4 | AGAUUUCAUUACCUCCUAC | GUAGGAGGUAAUGAAAUCU | MK | [2235-2253] |
| 5 | GCCACUACAUUGCCAUCAU | AUGAUGGCAAUGUAGUGGC | MK, Ms | [688-706] |
| 6 | CAGUGUGCCACAUUAAAUA | UAUUUAAUGUGGCACACUG | MK | [3225-3243] |
| 7 | UCAGUUUUUUGACUUCCUU | AAGGAAGUCAAAAAACUGA | MK | [2363-2381] |
| 8 | GUCAUUUCCAGAUUUCAUU | AAUGAAAUCUGGAAAUGAC | MK | [2226-2244] |
| 9 | ACAUUAAAUACCCGUGCAG | CUGCACGGGUAUUUAAUGU | MK | [3234-3252] |
| 10 | GCCAGUGUGCCACAUUAAA | UUUAAUGUGGCACACUGGC | MK | [3223-3241] |
| 11 | CCAACAGCAAGCGGAUUUU | AAAAUCCGCUUGCUGUUGG | MK | [2912-2930] |
| 12 | CUACUUGCCAUUCACCCAU | AUGGGUGAAUGGCAAGUAG | MK | [2250-2268] |
| 13 | GGGCCAUCUGGAACUAUAU | AUAUAGUUCCAGAUGGCCC | MK | [1525-1543] |
| 14 | GCAGAUGUCUCCCAAAAAG | CUUUUUGGGAGACAUCUGC | | [2683-2701] |
| 15 | UUGCCAUUCACCCAUCAAU | AUUGAUGGGUGAAUGGCAA | MK | [2254-2272] |
| 16 | CCUCCUACUUGCCAUUCAC | GUGAAUGGCAAGUAGGAGG | MK | [2246-2264] |
| 17 | ACCUCCUACUUGCCAUUCA | UGAAUGGCAAGUAGGAGGU | MK | [2245-2263] |
| 18 | GAAUUAAUGUGCCACAAGU | ACUUGUGGCACAUUAAUUC | | [2105-2123] |
| 19 | GGAAUUAAUGUGCCACAAG | CUUGUGGCACAUUAAUUCC | | [2104-2122] |
| 20 | UGCUACCAGUUCCAUAUGA | UCAUAUGGAACUGGUAGCA | MK | [3432-3450] |
| 21 | CCAGUGUUCAAGUGCAGAA | UUCUGCACUUGAACACUGG | MK | [3402-3420] |
| 22 | GCAUGUGAUGACUGUAAAU | AUUUACAGUCAUCACAUGC | | [3356-3374] |
| 23 | CGGAUUUUCUUGCAAGAUC | GAUCUUGCAAGAAAAUCCG | MK | [2923-2941] |
| 24 | CCUCUUUAUUUUGGUCCUU | AAGGACCAAAAUAAAGAGG | MK | [2535-2553] |
| 25 | GAAUCCUAGUUCAGUUUUU | AAAAACUGAACUAGGAUUC | MK | [2353-2371] |
| 26 | CCAUCAAUGUGAAAGUCAG | CUGACUUUCACAUUGAUGG | MK | [2265-2283] |
| 27 | GGCCAAGCUCGGAAUUAAU | AUUAAUUCCGAGCUUGGCC | | [2094-2112] |
| 28 | CAGUUCCAUAUGAUGAGAA | UUCUCAUCAUAUGGAACUG | MK | [3438-3456] |
| 29 | UCAUCACCAAGGAACACAU | AUGUGUUCCUUGGUGAUGA | MK | [868-886] |
| 30 | UGACUGUGAAUUACGACUU | AAGUCGUAAUUCACAGUCA | | [3097-3115] |
| 31 | GGAUUUUCUUGCAAGAUCA | UGAUCUUGCAAGAAAAUCC | MK | [2924-2942] |
| 32 | GCGGAUUUUCUUGCAAGAU | AUCUUGCAAGAAAAUCCGC | MK | [2922-2940] |
| 33 | CGCAGAUGUCUCCCAAAAA | UUUUUGGGAGACAUCUGCG | | [2682-2700] |
| 34 | CUCUUUAUUUUGGUCCUUU | AAAGGACCAAAAUAAAGAG | MK | [2536-2554] |
| 35 | AGAGGAGGGAGUAUUAGAU | AUCUAAUACUCCCUCCUCU | MK | [2509-2527] |
| 36 | CCUUUGCCCUUUUUCCCUU | AAGGGAAAAAGGGCAAAGG | | [2378-2396] |
| 37 | CGAAUCCUAGUUCAGUUUU | AAAACUGAACUAGGAUUCG | MK | [2352-2370] |
| 38 | CUGGAAUGGACAGUUCAUU | AAUGAACUGUCCAUUCCAG | MK | [1954-1972] |
| 39 | AGUUCCAUAUGAUGAGAAA | UUUCUCAUCAUAUGGAACU | MK | [3439-3457] |
| 40 | CCAGGGUUCUUGUUUGGAC | GUCCAAACAAGAACCCUGG | MK | [2760-2778] |
| 41 | UUACCUCCUACUUGCCAUU | AAUGGCAAGUAGGAGGUAA | MK | [2243-2261] |
| 42 | UCGGAAUUAAUGUGCCACA | UGUGGCACAUUAAUUCCGA | | [2102-2120] |
| 43 | GGCCAUCUGGAACUAUAUC | GAUAUAGUUCCAGAUGGCC | MK | [1526-1544] |
| 44 | GCCUCACCUACAAUACCAU | AUGGUAUUGUAGGUGAGGC | MK | [1468-1486] |
| 45 | AGAAAUCUUUGGCUUUGCU | AGCAAAGCCAAAGAUUUCU | MK | [3417-3435] |
| 46 | CUGUAGCAAAUGACUGUGA | UCACAGUCAUUUGCUACAG | MK | [3087-3105] |
| 47 | GCUCGGAAUUAAUGUGCCA | UGGCACAUUAAUUCCGAGC | | [2100-2118] |
| 48 | CCAAGCUCGGAAUUAAUGU | ACAUUAAUUCCGAGCUUGG | | [2096-2114] |
| 49 | GAAGAGUCCAGCACCAAAG | CUUUGGUGCUGGACUCUUC | | [172-190] |
| 50 | GGAUUAUACCUGGGAAGAC | GUCUUCCCAGGUAUAAUCC | MK, Rat, Ms | [1373-1391] |
| 51 | CUCUCCUCCUUCGUGUUUG | CAAACACGAAGGAGGAGAG | MK | [972-990] |
| 52 | GCUUUGCUACCAGUUCCAU | AUGGAACUGGUAGCAAAGC | MK | [3428-3446] |
| 53 | GACUUCUCUUGCCCUUCUU | AAGAAGGGCAAGAGAAGUC | | [3111-3129] |
| 54 | GUAGCAAAUGACUGUGAAU | AUUCACAGUCAUUUGCUAC | MK | [3089-3107] |
| 55 | CCAUCCCAGUAUCUCAUCU | AGAUGAGAUACUGGGAUGG | MK | [2998-3016] |
| 56 | AGCGGAUUUUCUUGCAAGA | UCUUGCAAGAAAAUCCGCU | MK | [2921-2939] |
| 57 | GGCACCAGGGUUCUUGUUU | AAACAAGAACCCUGGUGCC | MK | [2756-2774] |
| 58 | CUGAAGAGGAGGGAGUAUU | AAUACUCCCUCCUCUUCAG | MK | [2505-2523] |
| 59 | CUUUUUCCCUUUUCUCCAU | AUGGAGAAAAGGGAAAAAG | | [2386-2404] |
| 60 | CUUUGCCCUUUUUCCCUUU | AAAGGGAAAAAGGGCAAAG | | [2379-2397] |
| 61 | CAGUUUUUUGACUUCCUUU | AAAGGAAGUCAAAAAACUG | MK | [2364-2382] |
| 62 | CAAGCUCGGAAUUAAUGUG | CACAUUAAUUCCGAGCUUG | | [2097-2115] |
| 63 | ACUAUAUCCACUGCGUCUU | AAGACGCAGUGGAUAUAGU | MK | [1537-1555] |
| 64 | UGCACCCUGACUACUUUAC | GUAAAGUAGUCAGGGUGCA | MK | [607-625] |
| 65 | CAGACAUGCUGUGCUUUGU | ACAAAGCACAGCAUGUCUG | MK | [1282-1300] |
| 66 | CUGCACCCUGACUACUUUA | UAAAGUAGUCAGGGUGCAG | MK | [606-624] |
| 67 | ACCAGUUCCAUAUGAUGAG | CUCAUCAUAUGGAACUGGU | MK | [3436-3454] |
| 68 | CUAGAGGGCAUGUGAUGAC | GUCAUCACAUGCCCUCUAG | | [3349-3367] |
| 69 | CCCUUCUUCUAGCAGUCUG | CAGACUGCUAGAAGAAGGG | | [3122-3140] |
| 70 | UCCUCUUUAUUUUGGUCCU | AGGACCAAAAUAAAGAGGA | MK | [2534-2552] |
| 71 | GGAAUGGACAGUUCAUUGC | GCAAUGAACUGUCCAUUCC | MK | [1956-1974] |
| 72 | CACCCUGACUACUUUACCA | UGGUAAAGUAGUCAGGGUG | MK | [609-627] |
| 73 | UGCUGUGCUUUGUGGAAGA | UCUUCCACAAAGCACAGCA | MK | [1288-1306] |
| 74 | UGAUGAGAAAUAAACGUUC | GAACGUUUAUUUCUCAUCA | MK | [3448-3466] |
| 75 | UGAUGACUGUAAAUGUUCA | UGAACAUUUACAGUCAUCA | | [3361-3379] |
| 76 | CCGAUUCCAGGCACUUUCU | AGAAAGUGCCUGGAAUCGG | MK | [3070-3088] |
| 77 | ACAGCAAGCGGAUUUUCUU | AAGAAAAUCCGCUUGCUGU | MK | [2915-2933] |
| 78 | CCCACAAUGUCUGAAGCUG | CAGCUUCAGACAUUGUGGG | MK | [2868-2886] |
| 79 | CUAGAUGGCUUAGGUGGCA | UGCCACCUAAGCCAUCUAG | | [2712-2730] |
| 80 | UUGCCCUUUUUCCCUUUUC | GAAAAGGGAAAAAGGGCAA | | [2381-2399] |
| 81 | UCCUAGUUCAGUUUUUUGA | UCAAAAAACUGAACUAGGA | MK | [2356-2374] |
| 82 | CGAGUUUCUGCAGACUGGU | ACCAGUCUGCAGAAACUCG | MK | [755-773] |
| 83 | CCAGAUUUCAUUACCUCCU | AGGAGGUAAUGAAAUCUGG | MK | [2233-2251] |
| 84 | CGCCAUCAGUGUUCUUACC | GGUAAGAACACUGAUGGCG | MK | [717-735] |
| 85 | UGAGAAAUAAACGUUCGCU | AGCGAACGUUUAUUUCUCA | MK | [3451-3469] |
| 86 | UUCAAGUGCAGAAAUCUUU | AAAGAUUUCUGCACUUGAA | MK | [3408-3426] |
| 87 | GUGGUAUCCAGUGUUCAAG | CUUGAACACUGGAUACCAC | MK | [3395-3413] |
| 88 | UCUGGGUUCUAGAGGGCAU | AUGCCCUCUAGAACCCAGA | | [3341-3359] |
| 89 | GAGUAGACAACCUGGCAGU | ACUGCCAGGUUGUCUACUC | MK | [577-595] |
| 90 | CUCAGCGAGAUCAACAAGU | ACUUGUUGAUCUCGCUGAG | MK | [828-846] |
| 91 | CACAUUGCCUUCUGAAGAG | CUCUUCAGAAGGCAAUGUG | MK | [2494-2512] |
| 92 | CCUUUUCUCCAUGCUUAAU | AUUAAGCAUGGAGAAAAGG | MK | [2393-2411] |
| 93 | GCUUGUGUGUGAUGUGCAG | CUGCACAUCACACACAAGC | MK | [1906-1924] |
| 94 | GGCUUGUGUGUGAUGUGCA | UGCACAUCACACACAAGCC | MK | [1905-1923] |
| 95 | CCGCCAUCAGUGUUCUUAC | GUAAGAACACUGAUGGCGG | MK | [716-734] |
| 96 | CAGUGGGCUUGUGUGUGAU | AUCACACACAAGCCCACUG | | [1900-1918] |
| 97 | CUUUCGGAUAUGAGGACUU | AAGUCCUCAUAUCCGAAAG | MK | [1312-1330] |
| 98 | GAGAAAUAAACGUUCGCUG | CAGCGAACGUUUAUUUCUC | MK | [3452-3470] |
| 99 | CAGAAAUCUUUGGCUUUGC | GCAAAGCCAAAGAUUUCUG | MK | [3416-3434] |
| 100 | CCUUCUUCUAGCAGUCUGU | ACAGACUGCUAGAAGAAGG | | [3123-3141] |
| 101 | CCUUUAUGCUUGAGGUUCC | GGAACCUCAAGCAUAAAGG | MK | [2550-2568] |
| 102 | GGUCACAUUGCCUUCUGAA | UUCAGAAGGCAAUGUGACC | MK | [2491-2509] |
| 103 | UCUAGAGAGUUUGAGCCUU | AAGGCUCAAACUCUCUAGA | MK | [2468-2486] |
| 104 | GCAAACUCAGCGAGAUCAA | UUGAUCUCGCUGAGUUUGC | MK | [823-841] |
| 105 | UCAAUGUGAAAGUCAGGGU | ACCCUGACUUUCACAUUGA | MK | [2268-2286] |
| 106 | GCCAUCAGUGUUCUUACCU | AGGUAAGAACACUGAUGGC | MK | [718-736] |
| 107 | AGCUGCUGGAUGAGAAGUU | AACUUCUCAUCCAGCAGCU | MK, Rat, Ms | [1435-1453] |
| 108 | CGUUCGCUGAGGUUUUGUU | AACAAAACCUCAGCGAACG | MK | [3462-3480] |
| 109 | CCCAUCCCAGUAUCUCAUC | GAUGAGAUACUGGGAUGGG | MK | [2997-3015] |
| 110 | AGGCACCAGGGUUCUUGUU | AACAAGAACCCUGGUGCCU | MK | [2755-2773] |
| 111 | AGAGGGCACAGGAAAGAAG | CUUCUUUCCUGUGCCCUCU | MK | [2072-2090] |
| 112 | GUGGGCUUGUGUGUGAUGU | ACAUCACACACAAGCCCAC | MK | [1902-1920] |
| 113 | AGCCUCACCUACAAUACCA | UGGUAUUGUAGGUGAGGCU | MK | [1467-1485] |
| 114 | AGAGGGAGAAUUCUGUUCU | AGAACAGAAUUCUCCCUCU | | [2601-2619] |
| 115 | CAAUGUGAAAGUCAGGGUC | GACCCUGACUUUCACAUUG | MK | [2269-2287] |
| 116 | GGCUUAAGUGGGUUGCUUC | GAAGCAACCCACUUAAGCC | MK | [3031-3049] |
| 117 | GGAGGAGAGGGAGAAUUCU | AGAAUUCUCCCUCUCCUCC | | [2596-2614] |
| 118 | GCUGCUGGAUGAGAAGUUC | GAACUUCUCAUCCAGCAGC | MK, Ms | [1436-1454] |
| 119 | CGCUGAGGUUUUGUUUCAU | AUGAAACAAAACCUCAGCG | MK | [3466-3484] |
| 120 | GGGCACUGAAGAAAGGCAA | UUGCCUUUCUUCAGUGCCC | MK | [3165-3183] |
| 121 | CCCAAAAAGUUGAGCCUUU | AAAGGCUCAACUUUUUGGG | | [2693-2711] |
| 122 | GCUUAAUGGUGUGAGGCGU | ACGCCUCACACCAUUAAGC | MK | [2405-2423] |
| 123 | CCCUUUUCUCCAUGCUUAA | UUAAGCAUGGAGAAAAGGG | | [2392-2410] |
| 124 | UCCCUUUUCUCCAUGCUUA | UAAGCAUGGAGAAAAGGGA | | [2391-2409] |
| 125 | UGCCCUUUUUCCCUUUUCU | AGAAAAGGGAAAAAGGGCA | | [2382-2400] |
| 126 | UGACUUCCUUUGCCCUUUU | AAAAGGGCAAAGGAAGUCA | MK | [2372-2390] |
| 127 | ACCCUCCCUUUUCCUCACU | AGUGAGGAAAAGGGAGGGU | MK | [1937-1955] |
| 128 | GGAACCUCAAGGUCUAUAU | AUAUAGACCUUGAGGUUCC | MK | [1609-1627] |
| 129 | GCACUGAAGAAAGGCAAGG | CCUUGCCUUUCUUCAGUGC | MK | [3167-3185] |
| 130 | GCCCUUUUUCCCUUUUCUC | GAGAAAAGGGAAAAAGGGC | | [2383-2401] |
| 131 | CCCUGUCAUUUCCAGAUUU | AAAUCUGGAAAUGACAGGG | MK | [2222-2240] |
| 132 | GACAGUUCAUUGCACUGAC | GUCAGUGCAAUGAACUGUC | MK | [1962-1980] |
| 133 | CAGUGUUCAAGUGCAGAAA | UUUCUGCACUUGAACACUG | MK | [3403-3421] |
| 134 | UGCGCAAACUCAGCGAGAU | AUCUCGCUGAGUUUGCGCA | MK | [820-838] |
| 135 | AGAAGGUCCACGUGAACUU | AAGUUCACGUGGACCUUCU | | [1705-1723] |
| 136 | GUGUUCAAGUGCAGAAAUC | GAUUUCUGCACUUGAACAC | MK | [3405-3423] |
| 137 | GGGAGUGGUAUCCAGUGUU | AACACUGGAUACCACUCCC | MK | [3391-3409] |
| 138 | CCAAAAAGUUGAGCCUUUC | GAAAGGCUCAACUUUUUGG | MK | [2694-2712] |
| 139 | ACCCAUCAAUGUGAAAGUC | GACUUUCACAUUGAUGGGU | MK | [2263-2281] |
| 140 | ACAGUUCAUUGCACUGACU | AGUCAGUGCAAUGAACUGU | MK | [1963-1981] |
| 141 | GGACAGUUCAUUGCACUGA | UCAGUGCAAUGAACUGUCC | MK | [1961-1979] |
| 142 | CCCUUUUCCUCACUGGAAU | AUUCCAGUGAGGAAAAGGG | MK | [1942-1960] |
| 143 | CCCGAAGAAUGUACAACCU | AGGUUGUACAUUCUUCGGG | MK | [1660-1678] |
| 144 | AGGUCUAUAUCAAGACAGU | ACUGUCUUGAUAUAGACCU | MK | [1618-1636] |
| 145 | CUGACUACUUUACCAGCUU | AAGCUGGUAAAGUAGUCAG | MK | [613-631] |
| 146 | CCAGGAUUAUACCUGGGAA | UUCCCAGGUAUAAUCCUGG | MK, Ms | [1370-1388] |
| 147 | CCCAGACAUGCUGUGCUUU | AAAGCACAGCAUGUCUGGG | MK | [1280-1298] |
| 148 | GGCCAGUGUGCCACAUUAA | UUAAUGUGGCACACUGGCC | MK | [3222-3240] |
| 149 | CAGGCACUUUCUGUAGCAA | UUGCUACAGAAAGUGCCUG | MK | [3077-3095] |
| 150 | AGGUGACCAUGGCUACAUU | AAUGUAGCCAUGGUCACCU | MK | [2799-2817] |
| 151 | UUGACUUCCUUUGCCCUUU | AAAGGGCAAAGGAAGUCAA | MK | [2371-2389] |
| 152 | UGACACUCCAGGCAGCUUU | AAAGCUGCCUGGAGUGUCA | MK | [2193-2211] |
| 153 | CGAAGAAUGUACAACCUCU | AGAGGUUGUACAUUCUUCG | MK | [1662-1680] |
| 154 | CGGAACCUCAAGGUCUAUA | UAUAGACCUUGAGGUUCCG | MK | [1608-1626] |
| 155 | GCGUCUUUGGCAUCAGAUA | UAUCUGAUGCCAAAGACGC | MK, Ms | [1549-1567] |
| 156 | CCUACAAUACCAUCGCCAU | AUGGCGAUGGUAUUGUAGG | MK | [1474-1492] |
| 157 | GUGCAGAAAUCUUUGGCUU | AAGCCAAAGAUUUCUGCAC | MK | [3413-3431] |
| 158 | AGGGAGUGGUAUCCAGUGU | ACACUGGAUACCACUCCCU | MK | [3390-3408] |
| 159 | CACAUUAAAUACCCGUGCA | UGCACGGGUAUUUAAUGUG | MK | [3233-3251] |
| 160 | CCACAUUAAAUACCCGUGC | GCACGGGUAUUUAAUGUGG | MK | [3232-3250] |
| 161 | AGGCACUUUCUGUAGCAAA | UUUGCUACAGAAAGUGCCU | MK | [3078-3096] |
| 162 | CCGAAUCCUAGUUCAGUUU | AAACUGAACUAGGAUUCGG | MK | [2351-2369] |
| 163 | CCCGAAUCCUAGUUCAGUU | AACUGAACUAGGAUUCGGG | MK | [2350-2368] |
| 164 | CCUAAAAGCCUGUUCUGUU | AACAGAACAGGCUUUUAGG | MK | [2312-2330] |
| 165 | CCCUAAAAGCCUGUUCUGU | ACAGAACAGGCUUUUAGGG | MK | [2311-2329] |
| 166 | ACGUGAACUUGCUGCUCCU | AGGAGCAGCAAGUUCACGU | | [1714-1732] |
| 167 | GAAGAAUGUACAACCUCUU | AAGAGGUUGUACAUUCUUC | MK | [1663-1681] |
| 168 | GGAACUAUAUCCACUGCGU | ACGCAGUGGAUAUAGUUCC | MK | [1534-1552] |
| 169 | CCAUCUGGAACUAUAUCCA | UGGAUAUAGUUCCAGAUGG | MK | [1528-1546] |
| 170 | ACCUACAAUACCAUCGCCA | UGGCGAUGGUAUUGUAGGU | MK | [1473-1491] |
| 171 | UUGUGGAAGACCCUACUUU | AAAGUAGGGUCUUCCACAA | MK | [1297-1315] |
| 172 | UCGCUGAGGUUUUGUUUCA | UGAAACAAAACCUCAGCGA | MK | [3465-3483] |
| 173 | GGCUUUGCUACCAGUUCCA | UGGAACUGGUAGCAAAGCC | MK | [3427-3445] |
| 174 | AGUGUUCAAGUGCAGAAAU | AUUUCUGCACUUGAACACU | MK | [3404-3422] |
| 175 | GCCACAUUAAAUACCCGUG | CACGGGUAUUUAAUGUGGC | MK | [3231-3249] |
| 176 | UGGCCAGUGUGCCACAUUA | UAAUGUGGCACACUGGCCA | MK | [3221-3239] |
| 177 | GCACUUUCUGUAGCAAAUG | CAUUUGCUACAGAAAGUGC | MK | [3080-3098] |
| 178 | CAGGUGACCAUGGCUACAU | AUGUAGCCAUGGUCACCUG | MK | [2798-2816] |
| 179 | GCGAGAUCAACAAGUUGCU | AGCAACUUGUUGAUCUCGC | MK | [832-850] |
| 180 | GCCUUUGCUGGUCACAUUG | CAAUGUGACCAGCAAAGGC | MK | [2482-2500] |
| 181 | CACCCAUCAAUGUGAAAGU | ACUUUCACAUUGAUGGGUG | MK, Ms | [2262-2280] |
| 182 | GCCAUUCACCCAUCAAUGU | ACAUUGAUGGGUGAAUGGC | MK | [2256-2274] |
| 183 | CCUGUCAUUUCCAGAUUUC | GAAAUCUGGAAAUGACAGG | MK | [2223-2241] |
| 184 | CCACAAGUGUUGUGGCCUU | AAGGCCACAACACUUGUGG | MK | [2116-2134] |
| 185 | CCACCCGAAGAAUGUACAA | UUGUACAUUCUUCGGGUGG | MK | [1657-1675] |
| 186 | GCAUCAGAUAUGAUGACUA | UAGUCAUCAUAUCUGAUGC | MK | [1558-1576] |
| 187 | GGAAGACCCUACUUUCGGA | UCCGAAAGUAGGGUCUUCC | MK | [1301-1319] |
| 188 | GGAAGAGUCCAGCACCAAA | UUUGGUGCUGGACUCUUCC | | [171-189] |
| 189 | UGACUGUAAAUGUUCACUG | CAGUGAACAUUUACAGUCA | | [3364-3382] |
| 190 | CAAGCGGAUUUUCUUGCAA | UUGCAAGAAAAUCCGCUUG | MK | [2919-2937] |
| 191 | GCAAGCGGAUUUUCUUGCA | UGCAAGAAAAUCCGCUUGC | MK | [2918-2936] |
| 192 | UGGCUACAUUGCCAAACCU | AGGUUUGGCAAUGUAGCCA | MK | [2808-2826] |
| 193 | AGCCUUUCUAGAUGGCUUA | UAAGCCAUCUAGAAAGGCU | | [2705-2723] |
| 194 | CUCCCAAAAAGUUGAGCCU | AGGCUCAACUUUUUGGGAG | | [2691-2709] |
| 195 | AGAUGUCUCCCAAAAAGUU | AACUUUUUGGGAGACAUCU | | [2685-2703] |
| 196 | GUCCUUUAUGCUUGAGGUU | AACCUCAAGCAUAAAGGAC | MK | [2548-2566] |
| 197 | GAUUAUCUCUAGAGAGUUU | AAACUCUCUAGAGAUAAUC | MK | [2461-2479] |
| 198 | CCAUUCACCCAUCAAUGUG | CACAUUGAUGGGUGAAUGG | MK | [2257-2275] |
| 199 | CACUGACUCUGGGAUCUCA | UGAGAUCCCAGAGUCAGUG | MK | [1974-1992] |
| 200 | ACAAGGACUUCUCUGUCUG | CAGACAGAGAAGUCCUUGU | MK | [1832-1850] |
| 201 | AGGUCCACGUGAACUUGCU | AGCAAGUUCACGUGGACCU | | [1708-1726] |
| 202 | ACCUCAAGGUCUAUAUCAA | UUGAUAUAGACCUUGAGGU | MK | [1612-1630] |
| 203 | GACUACUUUACCAGCUUCU | AGAAGCUGGUAAAGUAGUC | MK | [615-633] |
| 204 | GAAGACCCUACUUUCGGAU | AUCCGAAAGUAGGGUCUUC | MK | [1302-1320] |
| 205 | ACCCAGACAUGCUGUGCUU | AAGCACAGCAUGUCUGGGU | MK | [1279-1297] |
| 206 | CACCAAGGAACACAUCCAG | CUGGAUGUGUUCCUUGGUG | MK | [872-890] |
| 207 | GAGCCUUUCUAGAUGGCUU | AAGCCAUCUAGAAAGGCUC | | [2704-2722] |
| 208 | AAAAAGUUGAGCCUUUCUA | UAGAAAGGCUCAACUUUUU | MK | [2696-2714] |
| 209 | GAGGAGGAGAGGGAGAAUU | AAUUCUCCCUCUCCUCCUC | | [2594-2612] |
| 210 | CUGGUCACAUUGCCUUCUG | CAGAAGGCAAUGUGACCAG | MK | [2489-2507] |
| 211 | AGAUUAUCUCUAGAGAGUU | AACUCUCUAGAGAUAAUCU | MK | [2460-2478] |
| 212 | AGUUCAGUUUUUUGACUUC | GAAGUCAAAAAACUGAACU | MK | [2360-2378] |
| 213 | GUGUCCAGUUCCCUAAAAG | CUUUUAGGGAACUGGACAC | MK | [2301-2319] |
| 214 | CCCAUCAAUGUGAAAGUCA | UGACUUUCACAUUGAUGGG | MK | [2264-2282] |
| 215 | UCACCCAUCAAUGUGAAAG | CUUUCACAUUGAUGGGUGA | MK | [2261-2279] |
| 216 | UCAGUGUUCUUACCUGGUA | UACCAGGUAAGAACACUGA | MK | [722-740] |
| 217 | GAGCACUUGGAGAUCCUAA | UUAGGAUCUCCAAGUGCUC | | [2015-2033] |
| 218 | AGAGCACUUGGAGAUCCUA | UAGGAUCUCCAAGUGCUCU | | [2014-2032] |
| 219 | CCCGCCAUCAGUGUUCUUA | UAAGAACACUGAUGGCGGG | MK | [715-733] |
| 220 | GGACUUCUCUGUCUGGAGA | UCUCCAGACAGAGAAGUCC | MK | [1836-1854] |
| 221 | CCCACAAGGACUUCUCUGU | ACAGAGAAGUCCUUGUGGG | MK | [1829-1847] |
| 222 | AGAAUGUACAACCUCUUCU | AGAAGAGGUUGUACAUUCU | MK | [1665-1683] |
| 223 | CCGAAGAAUGUACAACCUC | GAGGUUGUACAUUCUUCGG | MK | [1661-1679] |
| 224 | CAAGGUCUAUAUCAAGACA | UGUCUUGAUAUAGACCUUG | MK | [1616-1634] |
| 225 | GCGGAACCUCAAGGUCUAU | AUAGACCUUGAGGUUCCGC | MK | [1607-1625] |
| 226 | AGCGGAACCUCAAGGUCUA | UAGACCUUGAGGUUCCGCU | MK | [1606-1624] |
| 227 | CGUCUUUGGCAUCAGAUAU | AUAUCUGAUGCCAAAGACG | MK | [1550-1568] |
| 228 | GACCCUACUUUCGGAUAUG | CAUAUCCGAAAGUAGGGUC | MK | [1305-1323] |
| 229 | CUGUAAAUGUUCACUGGGU | ACCCAGUGAACAUUUACAG | | [3367-3385] |
| 230 | CUGUGAAUUACGACUUCUC | GAGAAGUCGUAAUUCACAG | | [3100-3118] |
| 231 | CCCAGUAUCUCAUCUGUCC | GGACAGAUGAGAUACUGGG | MK | [3002-3020] |
| 232 | GGCUACAUUGCCAAACCUC | GAGGUUUGGCAAUGUAGCC | MK | [2809-2827] |
| 233 | UCAAGGUCUAUAUCAAGAC | GUCUUGAUAUAGACCUUGA | MK | [1615-1633] |
| 234 | CGCCACUACAUUGCCAUCA | UGAUGGCAAUGUAGUGGCG | MK, Ms | [687-705] |
| 235 | CCCAGGAUUAUACCUGGGA | UCCCAGGUAUAAUCCUGGG | MK, Ms | [1369-1387] |
| 236 | ACCCUACUUUCGGAUAUGA | UCAUAUCCGAAAGUAGGGU | MK | [1306-1324] |
| 237 | GAGCUGGAGAAGUCAGAGA | UCUCUGACUUCUCCAGCUC | | [1215-1233] |
| 238 | AGGAACACAUCCAGGCCUU | AAGGCCUGGAUGUGUUCCU | MK | [877-895] |
| 239 | UGCCACAUUAAAUACCCGU | ACGGGUAUUUAAUGUGGCA | MK | [3230-3248] |
| 240 | GUGAAUUACGACUUCUCUU | AAGAGAAGUCGUAAUUCAC | | [3102-3120] |
| 241 | CCAUUUCUGCAGCCAGUGU | ACACUGGCUGCAGAAAUGG | | [2949-2967] |
| 242 | GGGUUCUUGUUUGGACCCU | AGGGUCCAAACAAGAACCC | MK | [2763-2781] |
| 243 | CAAAAAGUUGAGCCUUUCU | AGAAAGGCUCAACUUUUUG | MK | [2695-2713] |
| 244 | GGAGAAUUCUGUUCUCCCA | UGGGAGAACAGAAUUCUCC | | [2605-2623] |
| 245 | GCUGGUCACAUUGCCUUCU | AGAAGGCAAUGUGACCAGC | MK | [2488-2506] |
| 246 | UCAAGGACUACCUGCGGUU | AACCGCAGGUAGUCCUUGA | | [388-406] |
| 247 | CUAGUUCAGUUUUUUGACU | AGUCAAAAAACUGAACUAG | MK | [2358-2376] |
| 248 | UGGUCUGUGUGUCCAGUUC | GAACUGGACACACAGACCA | MK | [2293-2311] |
| 249 | CCAUCAGUGUUCUUACCUG | CAGGUAAGAACACUGAUGG | MK | [719-737] |
| 250 | AGACCCUUUUGUGUCCCAU | AUGGGACACAAAAGGGUCU | | [1862-1880] |
| 251 | AGAAGACCACCCGAAGAAU | AUUCUUCGGGUGGUCUUCU | MK | [1651-1669] |
| 252 | GCCAUCUGGAACUAUAUCC | GGAUAUAGUUCCAGAUGGC | MK | [1527-1545] |
| 253 | AGGGACCCGUUGAACAACU | AGUUGUUCAACGGGUCCCU | MK | [1080-1098] |
| 254 | UACGACUUCUCUUGCCCUU | AAGGGCAAGAGAAGUCGUA | | [3108-3126] |
| 255 | GGCUCAUCACCAAGGAACA | UGUUCCUUGGUGAUGAGCC | MK | [865-883] |
| 256 | ACAUUGCCAAACCUCUGAC | GUCAGAGGUUUGGCAAUGU | MK | [2813-2831] |
| 257 | ACCAUGGCUACAUUGCCAA | UUGGCAAUGUAGCCAUGGU | MK | [2804-2822] |
| 258 | GCCUUUCUAGAUGGCUUAG | CUAAGCCAUCUAGAAAGGC | | [2706-2724] |
| 259 | UCCCAAAAAGUUGAGCCUU | AAGGCUCAACUUUUUGGGA | | [2692-2710] |
| 260 | GGUCCUUUAUGCUUGAGGU | ACCUCAAGCAUAAAGGACC | MK | [2547-2565] |
| 261 | GAGAGUUUGAGCCUUUGCU | AGCAAAGGCUCAAACUCUC | MK | [2472-2490] |
| 262 | UUCAGUUUUUUGACUUCCU | AGGAAGUCAAAAAACUGAA | MK | [2362-2380] |
| 263 | CCAGUUCCCUAAAAGCCUG | CAGGCUUUUAGGGAACUGG | MK | [2305-2323] |
| 264 | UGUGUCCAGUUCCCUAAAA | UUUUAGGGAACUGGACACA | MK | [2300-2318] |
| 265 | AGCUGGAAGAGCACUUGGA | UCCAAGUGCUCUUCCAGCU | | [2007-2025] |
| 266 | UCCCUUUUCCUCACUGGAA | UUCCAGUGAGGAAAAGGGA | MK | [1941-1959] |
| 267 | CCACAAGGACUUCUCUGUC | GACAGAGAAGUCCUUGUGG | MK | [1830-1848] |
| 268 | ACAACCUCUUCUGGAGGCA | UGCCUCCAGAAGAGGUUGU | MK | [1672-1690] |
| 269 | CAUCAGAUAUGAUGACUAU | AUAGUCAUCAUAUCUGAUG | MK | [1559-1577] |
| 270 | GGCAUCAGAUAUGAUGACU | AGUCAUCAUAUCUGAUGCC | MK | [1557-1575] |
| 271 | CACUGCGUCUUUGGCAUCA | UGAUGCCAAAGACGCAGUG | MK, Ms | [1545-1563] |
| 272 | CAGGGCCAUCUGGAACUAU | AUAGUUCCAGAUGGCCCUG | MK | [1523-1541] |
| 273 | UGACUACUUUACCAGCUUC | GAAGCUGGUAAAGUAGUCA | MK | [614-632] |
| 274 | CCUGACUACUUUACCAGCU | AGCUGGUAAAGUAGUCAGG | MK | [612-630] |
| 275 | ACCAUGGCUACUCGCUGAU | AUCAGCGAGUAGCCAUGGU | MK, Rat, Ms | [1390-1408] |
| 276 | CAGGAUUAUACCUGGGAAG | CUUCCCAGGUAUAAUCCUG | MK, Rat, Ms | [1371-1389] |
| 277 | CCCUACUUUCGGAUAUGAG | CUCAUAUCCGAAAGUAGGG | MK | [1307-1325] |
| 278 | UCACCAAGGAACACAUCCA | UGGAUGUGUUCCUUGGUGA | MK | [871-889] |
| 279 | AGAAUUCUGUUCUCCCAGA | UCUGGGAGAACAGAAUUCU | | [2607-2625] |
| 280 | CUCCAUGCUUAAUGGUGUG | CACACCAUUAAGCAUGGAG | MK | [2399-2417] |
| 281 | UGCACUGACUCUGGGAUCU | AGAUCCCAGAGUCAGUGCA | MK | [1972-1990] |
| 282 | UGAACUUGCUGCUCCUGGA | UCCAGGAGCAGCAAGUUCA | MK | [1717-1735] |
| 283 | CGGAUAUGAGGACUUCACU | AGUGAAGUCCUCAUAUCCG | MK | [1316-1334] |
| 284 | UCGGAUAUGAGGACUUCAC | GUGAAGUCCUCAUAUCCGA | MK | [1315-1333] |
| 285 | GCUUUGUGGAAGACCCUAC | GUAGGGUCUUCCACAAAGC | MK | [1294-1312] |
| 286 | GGACCCGUUGAACAACUCU | AGAGUUGUUCAACGGGUCC | MK | [1082-1100] |
| 287 | UCCUUCGUGUUUGGCUGUG | CACAGCCAAACACGAAGGA | MK | [978-996] |
| 288 | CAGUUUGGAGGGCACUGAA | UUCAGUGCCCUCCAAACUG | MK | [3156-3174] |
| 289 | UGGCUCAUCACCAAGGAAC | GUUCCUUGGUGAUGAGCCA | MK | [864-882] |
| 290 | ACCGAUUCCAGGCACUUUC | GAAAGUGCCUGGAAUCGGU | MK | [3069-3087] |
| 291 | CAACAAGUUGCUGGCGCAU | AUGCGCCAGCAACUUGUUG | MK | [839-857] |
| 292 | UCAACAAGUUGCUGGCGCA | UGCGCCAGCAACUUGUUGA | MK | [838-856] |
| 293 | CCAUGGCUACAUUGCCAAA | UUUGGCAAUGUAGCCAUGG | MK | [2805-2823] |
| 294 | GAAUUCUGUUCUCCCAGAG | CUCUGGGAGAACAGAAUUC | | [2608-2626] |
| 295 | UGCUUGAGGUUCCAACCUG | CAGGUUGGAACCUCAAGCA | MK | [2556-2574] |
| 296 | GAGCCUUUGCUGGUCACAU | AUGUGACCAGCAAAGGCUC | MK | [2480-2498] |
| 297 | CAGAUUAUCUCUAGAGAGU | ACUCUCUAGAGAUAAUCUG | MK | [2459-2477] |
| 298 | GCAGAUUAUCUCUAGAGAG | CUCUCUAGAGAUAAUCUGC | MK | [2458-2476] |
| 299 | CCAUGCUUAAUGGUGUGAG | CUCACACCAUUAAGCAUGG | MK | [2401-2419] |
| 300 | GCCCGAAUCCUAGUUCAGU | ACUGAACUAGGAUUCGGGC | MK | [2349-2367] |
| 301 | CAAGUGUUGUGGCCUUCCU | AGGAAGGCCACAACACUUG | MK | [2119-2137] |
| 302 | UCACUGGAAUGGACAGUUC | GAACUGUCCAUUCCAGUGA | MK | [1951-1969] |
| 303 | GUCUUUGGCAUCAGAUAUG | CAUAUCUGAUGCCAAAGAC | MK | [1551-1569] |
| 304 | UGCGUCUUUGGCAUCAGAU | AUCUGAUGCCAAAGACGCA | MK, Ms | [1548-1566] |
| 305 | CUGCGUCUUUGGCAUCAGA | UCUGAUGCCAAAGACGCAG | MK, Ms | [1547-1565] |
| 306 | GCCUAUAGCCUCACCUACA | UGUAGGUGAGGCUAUAGGC | MK | [1461-1479] |
| 307 | ACCCUGACUACUUUACCAG | CUGGUAAAGUAGUCAGGGU | MK | [610-628] |
| 308 | GGAUAUGAGGACUUCACUC | GAGUGAAGUCCUCAUAUCC | MK | [1317-1335] |
| 309 | CGCUUUGAGCUGGAGAAGU | ACUUCUCCAGCUCAAAGCG | MK | [1209-1227] |
| 310 | UGCAGAAAUCUUUGGCUUU | AAAGCCAAAGAUUUCUGCA | MK | [3414-3432] |
| 311 | GCCCUUCUUCUAGCAGUCU | AGACUGCUAGAAGAAGGGC | MK | [3121-3139] |
| 312 | CGAUUCCAGGCACUUUCUG | CAGAAAGUGCCUGGAAUCG | MK | [3071-3089] |
| 313 | GACCAUGGCUACAUUGCCA | UGGCAAUGUAGCCAUGGUC | MK | [2803-2821] |
| 314 | GGUUCUUGUUUGGACCCUG | CAGGGUCCAAACAAGAACC | MK | [2764-2782] |
| 315 | UGGUCACAUUGCCUUCUGA | UCAGAAGGCAAUGUGACCA | MK | [2490-2508] |
| 316 | GUGUGUCCAGUUCCCUAAA | UUUAGGGAACUGGACACAC | MK | [2299-2317] |
| 317 | UGUGUGUCCAGUUCCCUAA | UUAGGGAACUGGACACACA | MK | [2298-2316] |
| 318 | CAGUGUUCUUACCUGGUAG | CUACCAGGUAAGAACACUG | MK | [723-741] |
| 319 | CAGAGCUCAAGGACUACCU | AGGUAGUCCUUGAGCUCUG | | [382-400] |
| 320 | CAGACCCUUUUGUGUCCCA | UGGGACACAAAAGGGUCUG | | [1861-1879] |
| 321 | UGGCAUCAGAUAUGAUGAC | GUCAUCAUAUCUGAUGCCA | MK | [1556-1574] |
| 322 | UCCACUGCGUCUUUGGCAU | AUGCCAAAGACGCAGUGGA | MK, Ms | [1543-1561] |
| 323 | CUAUAGCCUCACCUACAAU | AUUGUAGGUGAGGCUAUAG | MK | [1463-1481] |
| 324 | AGAAGUUCCAGGCAGCCUA | UAGGCUGCCUGGAACUUCU | MK | [1447-1465] |
| 325 | GACCGAUUCCAGGCACUUU | AAAGUGCCUGGAAUCGGUC | MK | [3068-3086] |
| 326 | CGAGAUCAACAAGUUGCUG | CAGCAACUUGUUGAUCUCG | MK | [833-851] |
| 327 | GAGGGAGAAUUCUGUUCUC | GAGAACAGAAUUCUCCCUC | | [2602-2620] |
| 328 | CCUUCCUGAACUGGGAAGU | ACUUCCCAGUUCAGGAAGG | MK | [2131-2149] |
| 329 | CAGCCUAUAGCCUCACCUA | UAGGUGAGGCUAUAGGCUG | MK | [1459-1477] |
| 330 | GAAGUUCCAGGCAGCCUAU | AUAGGCUGCCUGGAACUUC | MK | [1448-1466] |
| 331 | GGAUGAGAAGUUCCAGGCA | UGCCUGGAACUUCUCAUCC | MK | [1442-1460] |
| 332 | AGCUCAUUCAGGCUCUGGU | ACCAGAGCCUGAAUGAGCU | MK | [931-949] |
| 333 | UGCCCUUCUUCUAGCAGUC | GACUGCUAGAAGAAGGGCA | MK | [3120-3138] |
| 334 | CGCAAACUCAGCGAGAUCA | UGAUCUCGCUGAGUUUGCG | MK | [822-840] |
| 335 | CUGGUCUGUGUGUCCAGUU | AACUGGACACACAGACCAG | MK | [2292-2310] |
| 336 | AGGCAGCUUUGCCUUCUCU | AGAGAAGGCAAAGCUGCCU | MK | [2202-2220] |
| 337 | CACAAGUGUUGUGGCCUUC | GAAGGCCACAACACUUGUG | MK | [2117-2135] |
| 338 | UCCAUGCUUAAUGGUGUGA | UCACACCAUUAAGCAUGGA | MK | [2400-2418] |
| 339 | UGCUGGAUGAGAAGUUCCA | UGGAACUUCUCAUCCAGCA | MK, Ms | [1438-1456] |
| 340 | AGCACUUGGAGAUCCUAAG | CUUAGGAUCUCCAAGUGCU | | [2016-2034] |
| 341 | CUCACUGGAAUGGACAGUU | AACUGUCCAUUCCAGUGAG | MK | [1950-1968] |
| 342 | GCUGUGCUUUGUGGAAGAC | GUCUUCCACAAAGCACAGC | MK | [1289-1307] |
| 343 | GGCACUGAAGAAAGGCAAG | CUUGCCUUUCUUCAGUGCC | MK | [3166-3184] |
| 344 | CCUCACUGGAAUGGACAGU | ACUGUCCAUUCCAGUGAGG | MK | [1949-1967] |

TABLE B14
Hi95 19-mer oligomer pairs SESN2 - Sestrin 2

| | | | | Human-32454742: |
|---|---|---|---|---|
| No. | Sense | AntiSense | Other Sp | 354-1796 |

| | | | | |
|---|---|---|---|---|
| 1 | GGGAGUAUUAGAUUAUAAA | UUUAUAAUCUAAUACUCCC | Mnk | [2515-2533] |
| 2 | CCAUAUGAUGAGAAAUAAA | UUUAUUUCUCAUCAUAUGG | Mnk | [3443-3461] |
| 3 | CAGAGAAGGUCCACGUGAA | UUCACGUGGACCUUCUCUG | | [1702-1720] |
| 4 | GGCAUGUGAUGACUGUAAA | UUUACAGUCAUCACAUGCC | | [3355-3373] |
| 5 | GGGCCAAGCUCGGAAUUAA | UUAAUUCCGAGCUUGGCCC | | [2093-2111] |
| 6 | GGUUCUAGAGGGCAUGUGA | UCACAUGCCCUCUAGAACC | | [3345-3363] |
| 7 | GAUUAUAAAUCCUCUUUAU | AUAAAGAGGAUUUAUAAUC | Mnk | [2525-2543] |
| 8 | GGAGUAUUAGAUUAUAAAU | AUUUAUAAUCUAAUACUCC | Mnk | [2516-2534] |
| 9 | GAGGGAGUAUUAGAUUAUA | UAUAAUCUAAUACUCCCUC | Mnk | [2513-2531] |
| 10 | CCGCAGAUGUCUCCCAAAA | UUUUGGGAGACAUCUGCGG | | [2681-2699] |
| 11 | AGGGAGUAUUAGAUUAUAA | UUAUAAUCUAAUACUCCCU | Mnk | [2514-2532] |
| 12 | GGGCAUGUGAUGACUGUAA | UUACAGUCAUCACAUGCCC | | [3354-3372] |
| 13 | UGAAGAGGAGGGAGUAUUA | UAAUACUCCCUCCUCUUCA | Mnk | [2506-2524] |
| 14 | UGUAGCAAAUGACUGUGAA | UUCACAGUCAUUUGCUACA | Mnk | [3088-3106] |
| 15 | AAAUGACUGUGAAUUACGA | UCGUAAUUCACAGUCAUUU | Mnk | [3094-3112] |
| 16 | CAGAUAUGAUGACUAUGAU | AUCAUAGUCAUCAUAUCUG | Mnk | [1562-1580] |
| 17 | UGUGAUGUGCAGUCCCGAA | UUCGGGACUGCACAUCACA | Mnk | [1913-1931] |
| 18 | CAGAGGGCACAGGAAAGAA | UUCUUUCCUGUGCCCUCUG | Mnk | [2071-2089] |
| 19 | GGCACUUUCUGUAGCAAAU | AUUUGCUACAGAAAGUGCC | Mnk | [3079-3097] |
| 20 | AGUGGUAUCCAGUGUUCAA | UUGAACACUGGAUACCACU | Mnk | [3394-3412] |
| 21 | AGGUGUGCAGGAGAGAAGA | UCUUCUCUCCUGCACACCU | | [3306-3324] |
| 22 | CCUGCACCCUGACUACUUU | AAAGUAGUCAGGGUGCAGG | Mnk | [605-623] |
| 23 | AACUCAGCGAGAUCAACAA | UUGUUGAUCUCGCUGAGUU | Mnk | [826-844] |
| 24 | GCUACCAGUUCCAUAUGAU | AUCAUAUGGAACUGGUAGC | Mnk | [3433-3451] |
| 25 | CAUGGCUCAUCACCAAGGA | UCCUUGGUGAUGAGCCAUG | Mnk | [862-880] |
| 26 | GAAGGUGUGCAGGAGAGAA | UUCUCUCCUGCACACCUUC | | [3304-3322] |
| 27 | UCCAUAUGAUGAGAAAUAA | UUAUUUCUCAUCAUAUGGA | Mnk | [3442-3460] |
| 28 | CAUGCUGUGCUUUGUGGAA | UUCCACAAAGCACAGCAUG | Mnk | [1286-1304] |
| 29 | AGCAAAUGACUGUGAAUUA | UAAUUCACAGUCAUUUGCU | Mnk | [3091-3109] |
| 30 | CUUUGCUACCAGUUCCAUA | UAUGGAACUGGUAGCAAAG | Mnk | [3429-3447] |
| 31 | CCUACUUGCCAUUCACCCA | UGGGUGAAUGGCAAGUAGG | Mnk | [2249-2267] |
| 32 | CAGAUUUCAUUACCUCCUA | UAGGAGGUAAUGAAAUCUG | Mnk | [2234-2252] |
| 33 | CUGGAAGAGCACUUGGAGA | UCUCCAAGUGCUCUUCCAG | | [2009-2027] |
| 34 | AGGAGGGAGUAUUAGAUUA | UAAUCUAAUACUCCCUCCU | Mnk | [2511-2529] |
| 35 | GGAGGGAGUAUUAGAUUAU | AUAAUCUAAUACUCCCUCC | Mnk | [2512-2530] |
| 36 | CAAGAAGGUGUGCAGGAGA | UCUCCUGCACACCUUCUUG | | [3301-3319] |
| 37 | UCUUUAUUUUGGUCCUUUA | UAAAGGACCAAAAUAAAGA | Mnk | [2537-2555] |
| 38 | AAGAGGAGGGAGUAUUAGA | UCUAAUACUCCCUCCUCUU | Mnk | [2508-2526] |
| 39 | CCUAUAGCCUCACCUACAA | UUGUAGGUGAGGCUAUAGG | Mnk | [1462-1480] |
| 40 | GAAUGGACAGUUCAUUGCA | UGCAAUGAACUGUCCAUUC | Mnk | [1957-1975] |
| 41 | GUUCUAGAGGGCAUGUGAU | AUCACAUGCCCUCUAGAAC | | [3346-3364] |
| 42 | CUGUGUGUCCAGUUCCCUA | UAGGGAACUGGACACACAG | Mnk | [2297-2315] |
| 43 | GUUUGGAGGGCACUGAAGA | UCUUCAGUGCCCUCCAAAC | Mnk | [3158-3176] |
| 44 | AUAUGAUGACUAUGAUUAU | AUAAUCAUAGUCAUCAUAU | Mnk | [1565-1583] |
| 45 | CUUUAUUUUGGUCCUUUAU | AUAAAGGACCAAAAUAAAG | Mnk | [2538-2556] |
| 46 | CCUUUUUCCCUUUUCUCCA | UGGAGAAAAGGGAAAAAGG | | [2385-2403] |
| 47 | GCUACAUUGCCAAACCUCU | AGAGGUUUGGCAAUGUAGC | Mnk | [2810-2828] |
| 48 | UUUGGAGGGCACUGAAGAA | UUCUUCAGUGCCCUCCAAA | Mnk | [3159-3177] |
| 49 | CCAGUUCCAUAUGAUGAGA | UCUCAUCAUAUGGAACUGG | Mnk | [3437-3455] |
| 50 | GAAAUCUUUGGCUUUGCUA | UAGCAAAGCCAAAGAUUUC | Mnk | [3418-3436] |
| 51 | CUUUCUGUAGCAAAUGACU | AGUCAUUUGCUACAGAAAG | Mnk | [3083-3101] |
| 52 | UGUCAUUUCCAGAUUUCAU | AUGAAAUCUGGAAAUGACA | Mnk | [2225-2243] |
| 53 | CAUUGCACUGACUCUGGGA | UCCCAGAGUCAGUGCAAUG | Mnk | [1969-1987] |
| 54 | CUUUGAGCUGGAGAAGUCA | UGACUUCUCCAGCUCAAAG | | [1211-1229] |
| 55 | CCUCCUCUCUGACCAGUUU | AAACUGGUCAGAGAGGAGG | Mnk | [3143-3161] |
| 56 | UCAAGACAGUGGCCUGCUA | UAGCAGGCCACUGUCUUGA | Mnk | [1627-1645] |
| 57 | CUUGCCAUUCACCCAUCAA | UUGAUGGGUGAAUGGCAAG | Mnk | [2253-2271] |
| 58 | GAGGGCAUGUGAUGACUGU | ACAGUCAUCACAUGCCCUC | | [3352-3370] |
| 59 | AGGGCAUGUGAUGACUGUA | UACAGUCAUCACAUGCCCU | | [3353-3371] |
| 60 | CAAACCUCUGACUGCCACA | UGUGGCAGUCAGAGGUUUG | Mnk | [2820-2838] |
| 61 | AGGGCCAUCUGGAACUAUA | UAUAGUUCCAGAUGGCCCU | Mnk | [1524-1542] |
| 62 | GAGGAGGGAGUAUUAGAUU | AAUCUAAUACUCCCUCCUC | Mnk | [2510-2528] |
| 63 | CUAGAGAGUUUGAGCCUUU | AAAGGCUCAAACUCUCUAG | Mnk | [2469-2487] |
| 64 | UCAUUUCCAGAUUUCAUUA | UAAUGAAAUCUGGAAAUGA | Mnk | [2227-2245] |
| 65 | AGAUAUGAUGACUAUGAUU | AAUCAUAGUCAUCAUAUCU | Mnk | [1563-1581] |
| 66 | GGAGAGGGAGAAUUCUGUU | AACAGAAUUCUCCCUCUCC | | [2599-2617] |
| 67 | CCAGGCACUUUCUGUAGCA | UGCUACAGAAAGUGCCUGG | Mnk | [3076-3094] |
| 68 | AGAUUAUAAAUCCUCUUUA | UAAAGAGGAUUUAUAAUCU | Mnk | [2524-2542] |
| 69 | GUGUUGUGGCCUUCCUGAA | UUCAGGAAGGCCACAACAC | Mnk | [2122-2140] |
| 70 | AGAAAUAAACGUUCGCUGA | UCAGCGAACGUUUAUUUCU | Mnk | [3453-3471] |
| 71 | CACUGGAAUGGACAGUUCA | UGAACUGUCCAUUCCAGUG | Mnk | [1952-1970] |
| 72 | UUUGGUCCUUUAUGCUUGA | UCAAGCAUAAAGGACCAAA | Mnk | [2544-2562] |
| 73 | GAGUGGUAUCCAGUGUUCA | UGAACACUGGAUACCACUC | Mnk | [3393-3411] |
| 74 | AGAAGGUGUGCAGGAGAGA | UCUCUCCUGCACACCUUCU | | [3303-3321] |
| 75 | UGUGAUGACUGUAAAUGUU | AACAUUUACAGUCAUCACA | | [3359-3377] |
| 76 | CUUUCUAGAUGGCUUAGGU | ACCUAAGCCAUCUAGAAAG | | [2708-2726] |
| 77 | GCUACAUGACCUGACUCCU | AGGAGUCAGGUCAUGUAGC | Mnk | [1783-1801] |
| 78 | CAGAUGUCUCCCAAAAAGU | ACUUUUUGGGAGACAUCUG | | [2684-2702] |
| 79 | CUUUAUGCUUGAGGUUCCA | UGGAACCUCAAGCAUAAAG | Mnk | [2551-2569] |
| 80 | UACAUGACCUGACUCCUGA | UCAGGAGUCAGGUCAUGUA | Mnk | [1785-1803] |
| 81 | CACUUUCUGUAGCAAAUGA | UCAUUUGCUACAGAAAGUG | Mnk | [3081-3099] |
| 82 | AGAUGGAGAGCCGCUUUGA | UCAAAGCGGCUCUCCAUCU | Mnk | [1198-1216] |
| 83 | CUCAAGGUCUAUAUCAAGA | UCUUGAUAUAGACCUUGAG | Mnk | [1614-1632] |
| 84 | AGACCACCCGAAGAAUGUA | UACAUUCUUCGGGUGGUCU | Mnk | [1654-1672] |
| 85 | CCACUACAUUGCCAUCAUG | CAUGAUGGCAAUGUAGUGG | Ms, Mnk | [689-707] |
| 86 | CUUUGGCUUUGCUACCAGU | ACUGGUAGCAAAGCCAAAG | Mnk | [3423-3441] |
| 87 | GUUCCAUAUGAUGAGAAAU | AUUUCUCAUCAUAUGGAAC | Mnk | [3440-3458] |
| 88 | GCAAAUGACUGUGAAUUAC | GUAAUUCACAGUCAUUUGC | Mnk | [3092-3110] |
| 89 | GUUCAAGUGCAGAAAUCUU | AAGAUUUCUGCACUUGAAC | Mnk | [3407-3425] |
| 90 | AGUUCAUUGCACUGACUCU | AGAGUCAGUGCAAUGAACU | Mnk | [1965-1983] |
| 91 | CAUAUGAUGAGAAAUAAAC | GUUUAUUUCUCAUCAUAUG | Mnk | [3444-3462] |
| 92 | UUAUCUCUAGAGAGUUUGA | UCAAACUCUCUAGAGAUAA | Mnk | [2463-2481] |
| 93 | ACAGUGUGUGAGAGGAGGA | UCCUCCUCUCACACACUGU | Mnk | [2580-2598] |
| 94 | ACAUGCUGUGCUUUGUGGA | UCCACAAAGCACAGCAUGU | Mnk | [1285-1303] |
| 95 | GAGAAGACCACCCGAAGAA | UUCUUCGGGUGGUCUUCUC | Mnk | [1650-1668] |
| 96 | CAAACUCAGCGAGAUCAAC | GUUGAUCUCGCUGAGUUUG | Mnk | [824-842] |
| 97 | UGGGCUUGUGUGUGAUGUG | CACAUCACACACAAGCCCA | Mnk | [1903-1921] |
| 98 | GAUAUGAUGACUAUGAUUA | UAAUCAUAGUCAUCAUAUC | Mnk | [1564-1582] |
| 99 | ACUUGCCAUUCACCCAUCA | UGAUGGGUGAAUGGCAAGU | Mnk | [2252-2270] |
| 100 | CAUUACCUCCUACUUGCCA | UGGCAAGUAGGAGGUAAUG | Mnk | [2241-2259] |
| 101 | CAUUCACCCAUCAAUGUGA | UCACAUUGAUGGGUGAAUG | Mnk | [2258-2276] |
| 102 | GUAGGGAGUGGUAUCCAGU | ACUGGAUACCACUCCCUAC | Mnk | [3388-3406] |
| 103 | GAUUUUCUUGCAAGAUCAG | CUGAUCUUGCAAGAAAAUC | Mnk | [2925-2943] |
| 104 | AAGCGGAUUUUCUUGCAAG | CUUGCAAGAAAAUCCGCUU | Mnk | [2920-2938] |
| 105 | AAACUCAGCGAGAUCAACA | UGUUGAUCUCGCUGAGUUU | Mnk | [825-843] |
| 106 | GAUUUCAUUACCUCCUACU | AGUAGGAGGUAAUGAAAUC | Mnk | [2236-2254] |
| 107 | CUACUUUCGGAUAUGAGGA | UCCUCAUAUCCGAAAGUAG | Mnk | [1309-1327] |
| 108 | CAUGCUUAAUGGUGUGAGG | CCUCACACCAUUAAGCAUG | Mnk | [2402-2420] |
| 109 | UCAGCGAGAUCAACAAGUU | AACUUGUUGAUCUCGCUGA | Mnk | [829-847] |
| 110 | GUAUUAGAUUAUAAAUCCU | AGGAUUUAUAAUCUAAUAC | Mnk | [2519-2537] |
| 111 | GAGAAGCUGCGCAAACUCA | UGAGUUUGCGCAGCUUCUC | Mnk | [813-831] |
| 112 | AAAUCCUCUUUAUUUUGGU | ACCAAAAUAAAGAGGAUUU | Mnk | [2531-2549] |
| 113 | ACAUGACCUGACUCCUGAG | CUCAGGAGUCAGGUCAUGU | Mnk | [1786-1804] |
| 114 | AUUAGAUUAUAAAUCCUCU | AGAGGAUUUAUAAUCUAAU | Mnk | [2521-2539] |
| 115 | ACAUCCAGGCCUUGCUGAA | UUCAGCAAGGCCUGGAUGU | Mnk | [883-901] |
| 116 | UGGAGAAGUCAGAGAGCCU | AGGCUCUCUGACUUCUCCA | | [1219-1237] |
| 117 | AUGUGAUGACUGUAAAUGU | ACAUUUACAGUCAUCACAU | Mnk | [3358-3376] |
| 118 | CAGCGAGAUCAACAAGUUG | CAACUUGUUGAUCUCGCUG | Mnk | [830-848] |
| 119 | ACAAUACCAUCGCCAUGCA | UGCAUGGCGAUGGUAUUGU | Mnk | [1477-1495] |
| 120 | UGAGGACUUCACUCGGAGA | UCUCCGAGUGAAGUCCUCA | Mnk | [1322-1340] |
| 121 | UCACAUUGCCUUCUGAAGA | UCUUCAGAAGGCAAUGUGA | Mnk | [2493-2511] |
| 122 | CUGUCAUUUCCAGAUUUCA | UGAAAUCUGGAAAUGACAG | Mnk | [2224-2242] |
| 123 | AGCCUUUGCUGGUCACAUU | AAUGUGACCAGCAAAGGCU | Mnk | [2481-2499] |
| 124 | ACUGGAAUGGACAGUUCAU | AUGAACUGUCCAUUCCAGU | Mnk | [1953-1971] |
| 125 | ACUUGGAGAUCCUAAGGGA | UCCCUUAGGAUCUCCAAGU | | [2019-2037] |
| 126 | AGGAGAGGGAGAAUUCUGU | ACAGAAUUCUCCCUCUCCU | | [2598-2616] |
| 127 | GAAUGUACAACCUCUUCUG | CAGAAGAGGUUGUACAUUC | Mnk | [1666-1684] |
| 128 | GGGCUUAAGUGGGUUGCUU | AAGCAACCCACUUAAGCCC | Mnk | [3030-3048] |
| 129 | GAAAGUCAGGGUCACAGCU | AGCUGUGACCCUGACUUUC | Mnk | [2275-2293] |
| 130 | UCUAGAGGGCAUGUGAUGA | UCAUCACAUGCCCUCUAGA | | [3348-3366] |
| 131 | GAAGAGGAGGGAGUAUUAG | CUAAUACUCCCUCCUCUUC | Mnk | [2507-2525] |
| 132 | AAUACCAUCGCCAUGCACA | UGUGCAUGGCGAUGGUAUU | Mnk | [1479-1497] |
| 133 | CUAUAUCCACUGCGUCUUU | AAAGACGCAGUGGAUAUAG | Mnk | [1538-1556] |
| 134 | AUGGCUCAUCACCAAGGAA | UUCCUUGGUGAUGAGCCAU | Mnk | [863-881] |
| 135 | UCAGAGAAGGUCCACGUGA | UCACGUGGACCUUCUCUGA | | [1701-1719] |
| 136 | UGGAAGAGCACUUGGAGAU | AUCUCCAAGUGCUCUUCCA | | [2010-2028] |
| 137 | AGACCCUACUUUCGGAUAU | AUAUCCGAAAGUAGGGUCU | Mnk | [1304-1322] |
| 138 | AUUUCAUUACCUCCUACUU | AAGUAGGAGGUAAUGAAAU | Mnk | [2237-2255] |
| 139 | CUUUGUGGAAGACCCUACU | AGUAGGGUCUUCCACAAAG | Mnk | [1295-1313] |
| 140 | UGCUUUGUGGAAGACCCUA | UAGGGUCUUCCACAAAGCA | Mnk | [1293-1311] |
| 141 | AUUACCUCCUACUUGCCAU | AUGGCAAGUAGGAGGUAAU | Mnk | [2242-2260] |
| 142 | CUUUUGUGUCCCAUGCCCA | UGGGCAUGGGACACAAAAG | | [1867-1885] |
| 143 | GUGCUUUGUGGAAGACCCU | AGGGUCUUCCACAAAGCAC | Mnk | [1292-1310] |
| 144 | AAAGUUGAGCCUUUCUAGA | UCUAGAAAGGCUCAACUUU | | [2698-2716] |
| 145 | AAACGUUCGCUGAGGUUUU | AAAACCUCAGCGAACGUUU | Mnk | [3459-3477] |
| 146 | CCUACUUUCGGAUAUGAGG | CCUCAUAUCCGAAAGUAGG | Mnk | [1308-1326] |
| 147 | AUACCAUCGCCAUGCACAG | CUGUGCAUGGCGAUGGUAU | Mnk | [1480-1498] |
| 148 | AGAGAAGACCACCCGAAGA | UCUUCGGGUGGUCUUCUCU | Mnk | [1649-1667] |
| 149 | CCUCAAGGUCUAUAUCAAG | CUUGAUAUAGACCUUGAGG | Mnk | [1613-1631] |
| 150 | AUUCACCCAUCAAUGUGAA | UUCACAUUGAUGGGUGAAU | Mnk | [2259-2277] |
| 151 | CAAUACCAUCGCCAUGCAC | GUGCAUGGCGAUGGUAUUG | Mnk | [1478-1496] |
| 152 | AUUCCAGGCACUUUCUGUA | UACAGAAAGUGCCUGGAAU | Mnk | [3073-3091] |
| 153 | CAUCAAUGUGAAAGUCAGG | CCUGACUUUCACAUUGAUG | Mnk | [2266-2284] |
| 154 | AAUCCUAGUUCAGUUUUUU | AAAAAACUGAACUAGGAUU | Mnk | [2354-2372] |
| 155 | AAGCCACACCCUCCCUUUU | AAAAGGGAGGGUGUGGCUU | Mnk | [1930-1948] |
| 156 | UAUCCAGUGUUCAAGUGCA | UGCACUUGAACACUGGAUA | Mnk | [3399-3417] |
| 157 | ACUUCCGCCACUCAGAGAA | UUCUCUGAGUGGCGGAAGU | Ms, Mnk | [1690-1708] |
| 158 | GUUUCUGCAGACUGGUGGU | ACCACCAGUCUGCAGAAAC | Mnk | [758-776] |
| 159 | AGUGUUGUGGCCUUCCUGA | UCAGGAAGGCCACAACACU | Mnk | [2121-2139] |
| 160 | UUCUGAAGAGGAGGGAGUA | UACUCCCUCCUCUUCAGAA | Mnk | [2503-2521] |
| 161 | GAUGAGAAGUUCCAGGCAG | CUGCCUGGAACUUCUCAUC | Mnk | [1443-1461] |
| 162 | CUACAUUGCCAAACCUCUG | CAGAGGUUUGGCAAUGUAG | Mnk | [2811-2829] |
| 163 | GCUUUGAGCUGGAGAAGUC | GACUUCUCCAGCUCAAAGC | Mnk | [1210-1228] |
| 164 | UUCCAUAUGAUGAGAAAUA | UAUUUCUCAUCAUAUGGAA | Mnk | [3441-3459] |
| 165 | UGGUAUCCAGUGUUCAAGU | ACUUGAACACUGGAUACCA | Mnk | [3396-3414] |
| 166 | GCCAAGCUCGGAAUUAAUG | CAUUAAUUCCGAGCUUGGC | | [2095-2113] |
| 167 | ACACCCUCCCUUUUCCUCA | UGAGGAAAAGGGAGGGUGU | Mnk | [1935-1953] |
| 168 | CUACAAUACCAUCGCCAUG | CAUGGCGAUGGUAUUGUAG | Mnk | [1475-1493] |
| 169 | UCAGAUAUGAUGACUAUGA | UCAUAGUCAUCAUAUCUGA | Mnk | [1561-1579] |
| 170 | AGUUUGAGCCUUUGCUGGU | ACCAGCAAAGGCUCAAACU | Mnk | [2475-2493] |
| 171 | AUGGCUACUCGCUGAUCCA | UGGAUCAGCGAGUAGCCAU | Rat, Ms, Mnk | [1393-1411] |
| 172 | ACAACCUGGCAGUGGUGAU | AUCACCACUGCCAGGUUGU | Mnk | [583-601] |
| 173 | AUAUGAUGAGAAAUAAACG | CGUUUAUUUCUCAUCAUAU | Mnk | [3445-3463] |
| 174 | AGUGGGCUUGUGUGUGAUG | CAUCACACACAAGCCCACU | Mnk | [1901-1919] |
| 175 | UGAUGACUAUGAUUAUGGG | CCCAUAAUCAUAGUCAUCA | Mnk | [1568-1586] |
| 176 | UAUGAGGACUUCACUCGGA | UCCGAGUGAAGUCCUCAUA | Mnk | [1320-1338] |
| 177 | GCUCAUCACCAAGGAACAC | GUGUUCCUUGGUGAUGAGC | Mnk | [866-884] |
| 178 | AGUUGAGCCUUUCUAGAUG | CAUCUAGAAAGGCUCAACU | | [2700-2718] |
| 179 | GAGUAUUAGAUUAUAAAUC | GAUUUAUAAUCUAAUACUC | Mnk | [2517-2535] |
| 180 | UGGAGGGCACUGAAGAAAG | CUUUCUUCAGUGCCCUCCA | Mnk | [3161-3179] |
| 181 | GCACCAGGGUUCUUGUUUG | CAAACAAGAACCCUGGUGC | Mnk | [2757-2775] |
| 182 | CUAAAAGCCUGUUCUGUUG | CAACAGAACAGGCUUUUAG | Mnk | [2313-2331] |
| 183 | AGUUUGGAGGGCACUGAAG | CUUCAGUGCCCUCCAAACU | Mnk | [3157-3175] |
| 184 | AAGUUGAGCCUUUCUAGAU | AUCUAGAAAGGCUCAACUU | | [2699-2717] |
| 185 | UUCGGAUAUGAGGACUUCA | UGAAGUCCUCAUAUCCGAA | Mnk | [1314-1332] |
| 186 | CUACAUGACCUGACUCCUG | CAGGAGUCAGGUCAUGUAG | Mnk | [1784-1802] |
| 187 | UGGAAUGGACAGUUCAUUG | CAAUGAACUGUCCAUUCCA | Mnk | [1955-1973] |
| 188 | UUUGCUACCAGUUCCAUAU | AUAUGGAACUGGUAGCAAA | Mnk | [3430-3448] |
| 189 | GGAAGAGCACUUGGAGAUC | GAUCUCCAAGUGCUCUUCC | | [2011-2029] |
| 190 | AAGUUCCAGGCAGCCUAUA | UAUAGGCUGCCUGGAACUU | Mnk | [1449-1467] |
| 191 | AAAUCUUUGGCUUUGCUAC | GUAGCAAAGCCAAAGAUUU | Mnk | [3419-3437] |
| 192 | UCUGAAGAGGAGGGAGUAU | AUACUCCCUCCUCUUCAGA | Mnk | [2504-2522] |
| 193 | GAAAUAAACGUUCGCUGAG | CUCAGCGAACGUUUAUUUC | Mnk | [3454-3472] |
| 194 | AACAGCAAGCGGAUUUUCU | AGAAAAUCCGCUUGCUGUU | Mnk | [2914-2932] |
| 195 | UUUCUUGCAAGAUCAGGGA | UCCCUGAUCUUGCAAGAAA | Mnk | [2928-2946] |
| 196 | UAUGAUGAGAAAUAAACGU | ACGUUUAUUUCUCAUCAUA | Mnk | [3446-3464] |
| 197 | CUCUCUGACCAGUUUGGAG | CUCCAAACUGGUCAGAGAG | Mnk | [3147-3165] |
| 198 | UCAAGUGCAGAAAUCUUUG | CAAAGAUUUCUGCACUUGA | Mnk | [3409-3427] |
| 199 | ACAUGGCCGAGUUUCUGCA | UGCAGAAACUCGGCCAUGU | Mnk | [748-766] |
| 200 | GUCACAUUGCCUUCUGAAG | CUUCAGAAGGCAAUGUGAC | Mnk | [2492-2510] |
| 201 | GAUGAGAAAUAAACGUUCG | CGAACGUUUAUUUCUCAUC | Mnk | [3449-3467] |
| 202 | CUACCAGUUCCAUAUGAUG | CAUCAUAUGGAACUGGUAG | Mnk | [3434-3452] |
| 203 | UUGGAGGGCACUGAAGAAA | UUUCUUCAGUGCCCUCCAA | Mnk | [3160-3178] |
| 204 | UUCUGUAGCAAAUGACUGU | ACAGUCAUUUGCUACAGAA | Mnk | [3085-3103] |
| 205 | UUAGAUUAUAAAUCCUCUU | AAGAGGAUUUAUAAUCUAA | Mnk | [2522-2540] |
| 206 | CACAAUGUCUGAAGCUGCC | GGCAGCUUCAGACAUUGUG | Mnk | [2870-2888] |
| 207 | GAGUUUGAGCCUUUGCUGG | CCAGCAAAGGCUCAAACUC | Mnk | [2474-2492] |
| 208 | AAUGUUCACUGGGUGGGUA | UACCCACCCAGUGAACAUU | | [3372-3390] |
| 209 | CAUUGCCUUCUGAAGAGGA | UCCUCUUCAGAAGGCAAUG | Mnk | [2496-2514] |
| 210 | CAAAUGACUGUGAAUUACG | CGUAAUUCACAGUCAUUUG | Mnk | [3093-3111] |
| 211 | CCUAGUUCAGUUUUUUGAC | GUCAAAAAACUGAACUAGG | Mnk | [2357-2375] |
| 212 | CCUCUCUGACCAGUUUGGA | UCCAAACUGGUCAGAGAGG | Mnk | [3146-3164] |
| 213 | UACAUUGCCAAACCUCUGA | UCAGAGGUUUGGCAAUGUA | Mnk | [2812-2830] |
| 214 | CUGUUGCCCGAAUCCUAGU | ACUAGGAUUCGGGCAACAG | Mnk | [2344-2362] |
| 215 | ACUUCCGAGGCCGUGAAAA | UUUUCACGGCCUCGGAAGU | | [89-107] |
| 216 | AAGCAGCCAAGGACCGAUU | AAUCGGUCCUUGGCUGCUU | Mnk | [3057-3075] |
| 217 | AUGUCUCCCAAAAAGUUGA | UCAACUUUUUGGGAGACAU | | [2687-2705] |
| 218 | CUUGUGUGUGAUGUGCAGU | ACUGCACAUCACACACAAG | Mnk | [1907-1925] |
| 219 | CUUUGGCAUCAGAUAUGAU | AUCAUAUCUGAUGCCAAAG | Mnk | [1553-1571] |
| 220 | UGUUCAAGUGCAGAAAUCU | AGAUUUCUGCACUUGAACA | Mnk | [3406-3424] |
| 221 | CUCCCUUUUCCUCACUGGA | UCCAGUGAGGAAAAGGGAG | Mnk | [1940-1958] |
| 222 | UCUCCAUGCUUAAUGGUGU | ACACCAUUAAGCAUGGAGA | Mnk | [2398-2416] |
| 223 | UAGAGAGUUUGAGCCUUUG | CAAAGGCUCAAACUCUCUA | Mnk | [2470-2488] |
| 224 | GAGGAGAGGGAGAAUUCUG | CAGAAUUCUCCCUCUCCUC | | [2597-2615] |
| 225 | ACCACCCGAAGAAUGUACA | UGUACAUUCUUCGGGUGGU | Mnk | [1656-1674] |
| 226 | UGGAGAUCCUAAGGGACCA | UGGUCCCUUAGGAUCUCCA | | [2022-2040] |
| 227 | GCACCCUGACUACUUUACC | GGUAAAGUAGUCAGGGUGC | Mnk | [608-626] |
| 228 | CUCAUCACCAAGGAACACA | UGUGUUCCUUGGUGAUGAG | Mnk | [867-885] |
| 229 | CCACAAUGUCUGAAGCUGC | GCAGCUUCAGACAUUGUGG | Mnk | [2869-2887] |
| 230 | GAAGGUCCACGUGAACUUG | CAAGUUCACGUGGACCUUC | | [1706-1724] |
| 231 | CAAGGACUUCUCUGUCUGG | CCAGACAGAGAAGUCCUUG | Mnk | [1833-1851] |
| 232 | UAGAGGGCAUGUGAUGACU | AGUCAUCACAUGCCCUCUA | | [3350-3368] |
| 233 | AGGACUUCUCUGUCUGGAG | CUCCAGACAGAGAAGUCCU | Mnk | [1835-1853] |
| 234 | UUGAGCUGGAGAAGUCAGA | UCUGACUUCUCCAGCUCAA | Ms | [1213-1231] |
| 235 | GAACUAUAUCCACUGCGUC | GACGCAGUGGAUAUAGUUC | Mnk | [1535-1553] |
| 236 | AGCUGGAGAAGUCAGAGAG | CUCUCUGACUUCUCCAGCU | | [1216-1234] |
| 237 | CCUUUUCCUCACUGGAAUG | CAUUCCAGUGAGGAAAAGG | Mnk | [1943-1961] |
| 238 | AUGGCCGAGUUUCUGCAGA | UCUGCAGAAACUCGGCCAU | Mnk | [750-768] |
| 239 | AAAAGUUGAGCCUUUCUAG | CUAGAAAGGCUCAACUUUU | Mnk | [2697-2715] |
| 240 | AGAAGCUGCGCAAACUCAG | CUGAGUUUGCGCAGCUUCU | Mnk | [814-832] |
| 241 | GAUUCCAGGCACUUUCUGU | ACAGAAAGUGCCUGGAAUC | Mnk | [3072-3090] |
| 242 | GACUGUAAAUGUUCACUGG | CCAGUGAACAUUUACAGUC | | [3365-3383] |
| 243 | UCCAGUGUUCAAGUGCAGA | UCUGCACUUGAACACUGGA | Mnk | [3401-3419] |
| 244 | CUUCUGAAGAGGAGGGAGU | ACUCCCUCCUCUUCAGAAG | Mnk | [2502-2520] |
| 245 | ACUUUCUGUAGCAAAUGAC | GUCAUUUGCUACAGAAAGU | Mnk | [3082-3100] |
| 246 | AAGGUGUGCAGGAGAGAAG | CUUCUCUCCUGCACACCUU | | [3305-3323] |
| 247 | AAGAAGGUGUGCAGGAGAG | CUCUCCUGCACACCUUCUU | | [3302-3320] |
| 248 | CAUCACCAAGGAACACAUC | GAUGUGUUCCUUGGUGAUG | Mnk | [869-887] |
| 249 | GCAGAAAUCUUUGGCUUUG | CAAAGCCAAAGAUUUCUGC | Mnk | [3415-3433] |
| 250 | UUCUCUUGCCCUUCUUCUA | UAGAAGAAGGGCAAGAGAA | | [3114-3132] |
| 251 | CUUGAGGUUCCAACCUGGA | UCCAGGUUGGAACCUCAAG | Mnk | [2558-2576] |
| 252 | GCUCUCCUCCUUCGUGUUU | AAACACGAAGGAGGAGAGC | Mnk | [971-989] |
| 253 | UAUUUUGGUCCUUUAUGCU | AGCAUAAAGGACCAAAAUA | Mnk | [2541-2559] |
| 254 | UUCACCCAUCAAUGUGAAA | UUUCACAUUGAUGGGUGAA | Mnk | [2260-2278] |
| 255 | GAGUUUCUGCAGACUGGUG | CACCAGUCUGCAGAAACUC | Mnk | [756-774] |
| 256 | UGUCCAGUUCCCUAAAAGC | GCUUUUAGGGAACUGGACA | Mnk | [2302-2320] |
| 257 | GUUCGCUGAGGUUUUGUUU | AAACAAAACCUCAGCGAAC | Mnk | [3463-3481] |
| 258 | AAUAAACGUUCGCUGAGGU | ACCUCAGCGAACGUUUAUU | Mnk | [3456-3474] |
| 259 | UGUGAAUUACGACUUCUCU | AGAGAAGUCGUAAUUCACA | | [3101-3119] |
| 260 | AACUAUAUCCACUGCGUCU | AGACGCAGUGGAUAUAGUU | Mnk | [1536-1554] |
| 261 | ACUCAGCGAGAUCAACAAG | CUUGUUGAUCUCGCUGAGU | Mnk | [827-845] |
| 262 | AAGGACUUCUCUGUCUGGA | UCCAGACAGAGAAGUCCUU | Mnk | [1834-1852] |
| 263 | AUCUGGAACUAUAUCCACU | AGUGGAUAUAGUUCCAGAU | Mnk | [1530-1548] |
| 264 | AAAUGUUCACUGGGUGGGU | ACCCACCCAGUGAACAUUU | | [3371-3389] |
| 265 | AGUGUGCCACAUUAAAUAC | GUAUUUAAUGUGGCACACU | Mnk | [3226-3244] |
| 266 | UAGACAACCUGGCAGUGGU | ACCACUGCCAGGUUGUCUA | Mnk | [580-598] |
| 267 | AGUUCCAGGCAGCCUAUAG | CUAUAGGCUGCCUGGAACU | Mnk | [1450-1468] |
| 268 | AGAAGUCAGAGAGCCUGCU | AGCAGGCUCUCUGACUUCU | | [1222-1240] |
| 269 | UCGUGUUUGGCUGUGGCAU | AUGCCACAGCCAAACACGA | Mnk | [982-1000] |
| 270 | UCUCCUCCUUCGUGUUUGG | CCAAACACGAAGGAGGAGA | Mnk | [973-991] |
| 271 | ACUCAGAGAAGGUCCACGU | ACGUGGACCUUCUCUGAGU | | [1699-1717] |
| 272 | UUAAUGGUGUGAGGCGUCA | UGACGCCUCACACCAUUAA | Mnk | [2407-2425] |
| 273 | GCUGGAUGAGAAGUUCCAG | CUGGAACUUCUCAUCCAGC | Ms, Mnk | [1439-1457] |
| 274 | AGGGCACUGAAGAAAGGCA | UGCCUUUCUUCAGUGCCCU | Mnk | [3164-3182] |
| 275 | UUCCGGGCCCAGGAUUAUA | UAUAAUCCUGGGCCCGGAA | Mnk | [1362-1380] |
| 276 | AGAGUUUGAGCCUUUGCUG | CAGCAAAGGCUCAAACUCU | Mnk | [2473-2491] |
| 277 | UGGGUUCUAGAGGGCAUGU | ACAUGCCCUCUAGAACCCA | | [3343-3361] |
| 278 | AUGAGAAGUUCCAGGCAGC | GCUGCCUGGAACUUCUCAU | Mnk | [1444-1462] |
| 279 | UGAGCUGGAGAAGUCAGAG | CUCUGACUUCUCCAGCUCA | | [1214-1232] |
| 280 | GAGAUCCUAAGGGACCACA | UGUGGUCCCUUAGGAUCUC | Mnk | [2024-2042] |
| 281 | CAGUUCCCUAAAAGCCUGU | ACAGGCUUUUAGGGAACUG | Mnk | [2306-2324] |
| 282 | AGAGAAGGUCCACGUGAAC | GUUCACGUGGACCUUCUCU | | [1703-1721] |
| 283 | UGUGAAAGUCAGGGUCACA | UGUGACCCUGACUUUCACA | Mnk | [2272-2290] |
| 284 | UGUGGAAGACCCUACUUUC | GAAAGUAGGGUCUUCCACA | Mnk | [1298-1316] |
| 285 | AGUAUUAGAUUAUAAAUCC | GGAUUUAUAAUCUAAUACU | Mnk | [2518-2536] |
| 286 | AAUUAAUGUGCCACAAGUG | CACUUGUGGCACAUUAAUU | | [2106-2124] |
| 287 | CAGCAAGCGGAUUUUCUUG | CAAGAAAAUCCGCUUGCUG | Mnk | [2916-2934] |
| 288 | UCCCUAAAAGCCUGUUCUG | CAGAACAGGCUUUUAGGGA | Mnk | [2310-2328] |
| 289 | AGACAUGCUGUGCUUUGUG | CACAAAGCACAGCAUGUCU | Mnk | [1283-1301] |
| 290 | CUACAUUGCCAUCAUGGCU | AGCCAUGAUGGCAAUGUAG | Ms, Mnk | [692-710] |
| 291 | UGUACAACCUCUUCUGGAG | CUCCAGAAGAGGUUGUACA | Mnk | [1669-1687] |
| 292 | AUGAGGACUUCACUCGGAG | CUCCGAGUGAAGUCCUCAU | Mnk | [1321-1339] |
| 293 | CAACAGCAAGCGGAUUUUC | GAAAAUCCGCUUGCUGUUG | Mnk | [2913-2931] |
| 294 | AUUAAUGUGCCACAAGUGU | ACACUUGUGGCACAUUAAU | | [2107-2125] |
| 295 | AUGACCUGACUCCUGAGCA | UGCUCAGGAGUCAGGUCAU | Mnk | [1788-1806] |
| 296 | GACUGUGAAUUACGACUUC | GAAGUCGUAAUUCACAGUC | | [3098-3116] |
| 297 | AUGACUGUGAAUUACGACU | AGUCGUAAUUCACAGUCAU | Mnk | [3096-3114] |
| 298 | CCAGACAUGCUGUGCUUUG | CAAAGCACAGCAUGUCUGG | Mnk | [1281-1299] |
| 299 | GGUAUCCAGUGUUCAAGUG | CACUUGAACACUGGAUACC | Mnk | [3397-3415] |
| 300 | AAUGACUGUGAAUUACGAC | GUCGUAAUUCACAGUCAUU | Mnk | [3095-3113] |
| 301 | AAUGUGAAAGUCAGGGUCA | UGACCCUGACUUUCACAUU | Mnk | [2270-2288] |
| 302 | CACAAGGACUUCUCUGUCU | AGACAGAGAAGUCCUUGUG | Mnk | [1831-1849] |
| 303 | UAAUGUGCCACAAGUGUUG | CAACACUUGUGGCACAUUA | Mnk | [2109-2127] |
| 304 | UAUAGCCUCACCUACAAUA | UAUUGUAGGUGAGGCUAUA | Mnk | [1464-1482] |
| 305 | UUAAUGUGCCACAAGUGUU | AACACUUGUGGCACAUUAA | Mnk | [2108-2126] |
| 306 | CCUCACCUACAAUACCAUC | GAUGGUAUUGUAGGUGAGG | Mnk | [1469-1487] |
| 307 | UACCAGUUCCAUAUGAUGA | UCAUCAUAUGGAACUGGUA | Mnk | [3435-3453] |
| 308 | UCAUUGCACUGACUCUGGG | CCCAGAGUCAGUGCAAUGA | Mnk | [1968-1986] |
| 309 | AACACAUCCAGGCCUUGCU | AGCAAGGCCUGGAUGUGUU | Mnk | [880-898] |
| 310 | UGAAAGUCAGGGUCACAGC | GCUGUGACCCUGACUUUCA | Mnk | [2274-2292] |
| 311 | GGAGUGGUAUCCAGUGUUC | GAACACUGGAUACCACUCC | Mnk | [3392-3410] |
| 312 | AGGAGGAGAGGGAGAAUUC | GAAUUCUCCCUCUCCUCCU | | [2595-2613] |
| 313 | UCCAGUUCCCUAAAAGCCU | AGGCUUUUAGGGAACUGGA | Mnk | [2304-2322] |
| 314 | AAGAGCACUUGGAGAUCCU | AGGAUCUCCAAGUGCUCUU | | [2013-2031] |
| 315 | UGCUACCCAGAGAAGACCA | UGGUCUUCUCUGGGUAGCA | Mnk | [1641-1659] |
| 316 | CAUUUCCAGAUUUCAUUAC | GUAAUGAAAUCUGGAAAUG | Mnk | [2228-2246] |
| 317 | CGACUUCUCUUGCCCUUCU | AGAAGGGCAAGAGAAGUCG | | [3110-3128] |
| 318 | AGAGGGCAUGUGAUGACUG | CAGUCAUCACAUGCCCUCU | | [3351-3369] |
| 319 | UAGCAAAUGACUGUGAAUU | AAUUCACAGUCAUUUGCUA | Mnk | [3090-3108] |
| 320 | GACAUGCUGUGCUUUGUGG | CCACAAAGCACAGCAUGUC | Mnk | [1284-1302] |
| 321 | GUUCUUACCUGGUAGGCUC | GAGCCUACCAGGUAAGAAC | Mnk | [727-745] |
| 322 | AGGAUUAUACCUGGGAAGA | UCUUCCCAGGUAUAAUCCU | Rat, Ms, Mnk | [1372-1390] |
| 323 | CACUACAUUGCCAUCAUGG | CCAUGAUGGCAAUGUAGUG | Ms, Mnk | [690-708] |
| 324 | UUGCUUCCAGGCAGAAGCA | UGCUUCUGCCUGGAAGCAA | Mnk | [3043-3061] |
| 325 | AUUGCACUGACUCUGGGAU | AUCCCAGAGUCAGUGCAAU | Mnk | [1970-1988] |
| 326 | AAUGGACAGUUCAUUGCAC | GUGCAAUGAACUGUCCAUU | Mnk | [1958-1976] |
| 327 | AUGAUGAGAAAUAAACGUU | AACGUUUAUUUCUCAUCAU | Mnk | [3447-3465] |
| 328 | UCUUUGGCAUCAGAUAUGA | UCAUAUCUGAUGCCAAAGA | Mnk | [1552-1570] |
| 329 | UAAGUGGGUUGCUUCCAGG | CCUGGAAGCAACCCACUUA | Mnk | [3035-3053] |
| 330 | GUUGUGGCCUUCCUGAACU | AGUUCAGGAAGGCCACAAC | Mnk | [2124-2142] |
| 331 | UUGGCAUCAGAUAUGAUGA | UCAUCAUAUCUGAUGCCAA | Mnk | [1555-1573] |
| 332 | AUAAAUCCUCUUUAUUUUG | CAAAAUAAAGAGGAUUUAU | Mnk | [2529-2547] |
| 333 | GAUGUCUCCCAAAAAGUUG | CAACUUUUUGGGAGACAUC | | [2686-2704] |
| 334 | CAAGUGCAGAAAUCUUUGG | CCAAAGAUUUCUGCACUUG | Mnk | [3410-3428] |
| 335 | CUCCUACUUGCCAUUCACC | GGUGAAUGGCAAGUAGGAG | Mnk | [2247-2265] |
| 336 | GACCCUUUUGUGUCCCAUG | CAUGGGACACAAAAGGGUC | | [1863-1881] |
| 337 | CACCAGGGUUCUUGUUUGG | CCAAACAAGAACCCUGGUG | Mnk | [2758-2776] |
| 338 | UGCAAGAAGGUGUGCAGGA | UCCUGCACACCUUCUUGCA | | [3299-3317] |
| 339 | ACUUCUCUGUCUGGAGACA | UGUCUCCAGACAGAGAAGU | Mnk | [1838-1856] |
| 340 | AAGACCCUACUUUCGGAUA | UAUCCGAAAGUAGGGUCUU | Mnk | [1303-1321] |
| 341 | UUUCAUUACCUCCUACUUG | CAAGUAGGAGGUAAUGAAA | Mnk | [2238-2256] |
| 342 | AUGGACAGUUCAUUGCACU | AGUGCAAUGAACUGUCCAU | Mnk | [1959-1977] |
| 343 | AUGUACAACCUCUUCUGGA | UCCAGAAGAGGUUGUACAU | Mnk | [1668-1686] |
| 344 | UUCUAGAGGGCAUGUGAUG | CAUCACAUGCCCUCUAGAA | | [3347-3365] |
| 345 | UUAUUUUGGUCCUUUAUGC | GCAUAAAGGACCAAAAUAA | Mnk | [2540-2558] |
| 346 | CUUCCUUUGCCCUUUUUCC | GGAAAAAGGGCAAAGGAAG | | [2375-2393] |
| 347 | AUUAUACCUGGGAAGACCA | UGGUCUUCCCAGGUAUAAU | Rat, Ms, Mnk | [1375-1393] |
| 348 | CAUCUGGAACUAUAUCCAC | GUGGAUAUAGUUCCAGAUG | Mnk | [1529-1547] |
| 349 | CUUAAGUGGGUUGCUUCCA | UGGAAGCAACCCACUUAAG | Mnk | [3033-3051] |
| 350 | UUUAUGCUUGAGGUUCCAA | UUGGAACCUCAAGCAUAAA | Mnk | [2552-2570] |
| 351 | CAUCAGUGUUCUUACCUGG | CCAGGUAAGAACACUGAUG | Mnk | [720-738] |
| 352 | CCCUUUUUCCCUUUUCUCC | GGAGAAAAGGGAAAAAGGG | | [2384-2402] |
| 353 | UUGCCCGAAUCCUAGUUCA | UGAACUAGGAUUCGGGCAA | Mnk | [2347-2365] |
| 354 | AUCUGGGUUCUAGAGGGCA | UGCCCUCUAGAACCCAGAU | | [3340-3358] |
| 355 | AGAUCAACAAGUUGCUGGC | GCCAGCAACUUGUUGAUCU | Mnk | [835-853] |
| 356 | AUCCAGGCCUUGCUGAAGA | UCUUCAGCAAGGCCUGGAU | Ms, Mnk | [885-903] |
| 357 | AAGGAACACAUCCAGGCCU | AGGCCUGGAUGUGUUCCUU | Mnk | [876-894] |
| 358 | GCACUUGGAGAUCCUAAGG | CCUUAGGAUCUCCAAGUGC | | [2017-2035] |
| 359 | GUGCCACAUUAAAUACCCG | CGGGUAUUUAAUGUGGCAC | Mnk | [3229-3247] |
| 360 | UGCCAUUCACCCAUCAAUG | CAUUGAUGGGUGAAUGGCA | Mnk | [2255-2273] |
| 361 | AUUUUGGUCCUUUAUGCUU | AAGCAUAAAGGACCAAAAU | Mnk | [2542-2560] |
| 362 | UUCAUUACCUCCUACUUGC | GCAAGUAGGAGGUAAUGAA | Mnk | [2239-2257] |
| 363 | AAGACCACCCGAAGAAUGU | ACAUUCUUCGGGUGGUCUU | Mnk | [1653-1671] |
| 364 | ACCUCUUCUGGAGGCACUU | AAGUGCCUCCAGAAGAGGU | Mnk | [1675-1693] |
| 365 | UCUAGAUGGCUUAGGUGGC | GCCACCUAAGCCAUCUAGA | | [2711-2729] |
| 366 | AGUUUUUUGACUUCCUUUG | CAAAGGAAGUCAAAAAACU | Mnk | [2365-2383] |
| 367 | CAUUGCCAAACCUCUGACU | AGUCAGAGGUUUGGCAAUG | Mnk | [2814-2832] |
| 368 | GCUUAAGUGGGUUGCUUCC | GGAAGCAACCCACUUAAGC | Mnk | [3032-3050] |
| 369 | ACCAGGGUUCUUGUUUGGA | UCCAAACAAGAACCCUGGU | Mnk | [2759-2777] |
| 370 | AGCUCGGAAUUAAUGUGCC | GGCACAUUAAUUCCGAGCU | | [2099-2117] |
| 371 | AAGCCUGUUCUGUUGGGCA | UGCCCAACAGAACAGGCUU | Mnk | [2317-2335] |
| 372 | UGAGCCUUUGCUGGUCACA | UGUGACCAGCAAAGGCUCA | Mnk | [2479-2497] |
| 373 | AGUGCAGAAAUCUUUGGCU | AGCCAAAGAUUUCUGCACU | Mnk | [3412-3430] |
| 374 | UCCUUUGCCCUUUUUCCCU | AGGGAAAAAGGGCAAAGGA | | [2377-2395] |
| 375 | UUAAGUGGGUUGCUUCCAG | CUGGAAGCAACCCACUUAA | Mnk | [3034-3052] |
| 376 | AUUUCUGCAGCCAGUGUCU | AGACACUGGCUGCAGAAAU | | [2951-2969] |
| 377 | UUUCCAGAUUUCAUUACCU | AGGUAAUGAAAUCUGGAAA | Mnk | [2230-2248] |
| 378 | GACCACCCGAAGAAUGUAC | GUACAUUCUUCGGGUGGUC | Mnk | [1655-1673] |
| 379 | AUAAACGUUCGCUGAGGUU | AACCUCAGCGAACGUUUAU | Mnk | [3457-3475] |
| 380 | UUCGUGUUUGGCUGUGGCA | UGCCACAGCCAAACACGAA | Mnk | [981-999] |
| 381 | CCCUGACUACUUUACCAGC | GCUGGUAAAGUAGUCAGGG | Mnk | [611-629] |
| 382 | UUUUGGUCCUUUAUGCUUG | CAAGCAUAAAGGACCAAAA | Mnk | [2543-2561] |
| 383 | GUGAUGACUGUAAAUGUUC | GAACAUUUACAGUCAUCAC | | [3360-3378] |
| 384 | UACUUGCCAUUCACCCAUC | GAUGGGUGAAUGGCAAGUA | Mnk | [2251-2269] |
| 385 | UAGGGAGUGGUAUCCAGUG | CACUGGAUACCACUCCCUA | Mnk | [3389-3407] |
| 386 | GUUCUUGUUUGGACCCUGC | GCAGGGUCCAAACAAGAAC | Mnk | [2765-2783] |
| 387 | UCUGGAACUAUAUCCACUG | CAGUGGAUAUAGUUCCAGA | Mnk | [1531-1549] |
| 388 | UUCUGCAGACUGGUGGUGA | UCACCACCAGUCUGCAGAA | Mnk | [760-778] |
| 389 | AAGUGUUGUGGCCUUCCUG | CAGGAAGGCCACAACACUU | Mnk | [2120-2138] |
| 390 | GUUCAUUGCACUGACUCUG | CAGAGUCAGUGCAAUGAAC | Mnk | [1966-1984] |
| 391 | GACCCGUUGAACAACUCUG | CAGAGUUGUUCAACGGGUC | Mnk | [1083-1101] |
| 392 | CUCUAGAGAGUUUGAGCCU | AGGCUCAAACUCUCUAGAG | Mnk | [2467-2485] |
| 393 | UAUGAUGACUAUGAUUAUG | CAUAAUCAUAGUCAUCAUA | Mnk | [1566-1584] |
| 394 | AUCCAGUGUUCAAGUGCAG | CUGCACUUGAACACUGGAU | Mnk | [3400-3418] |
| 395 | UGGACAGUUCAUUGCACUG | CAGUGCAAUGAACUGUCCA | Mnk | [1960-1978] |
| 396 | CUCCUUCGUGUUUGGCUGU | ACAGCCAAACACGAAGGAG | Mnk | [977-995] |
| 397 | CUUGGAGAUCCUAAGGGAC | GUCCCUUAGGAUCUCCAAG | | [2020-2038] |
| 398 | AAUCCUCUUUAUUUUGGUC | GACCAAAAUAAAGAGGAUU | Mnk | [2532-2550] |
| 399 | GAAGACCACCCGAAGAAUG | CAUUCUUCGGGUGGUCUUC | Mnk | [1652-1670] |
| 400 | AUAUGAGGACUUCACUCGG | CCGAGUGAAGUCCUCAUAU | Mnk | [1319-1337] |
| 401 | CACUGAAGAAAGGCAAGGG | CCCUUGCCUUUCUUCAGUG | Mnk | [3168-3186] |
| 402 | CUCCUCUCUGACCAGUUUG | CAAACUGGUCAGAGAGGAG | Mnk | [3144-3162] |
| 403 | UUGUGUGUGAUGUGCAGUC | GACUGCACAUCACACACAA | Mnk | [1908-1926] |
| 404 | CAUUAAAUACCCGUGCAGG | CCUGCACGGGUAUUUAAUG | Mnk | [3235-3253] |
| 405 | UUAUACCUGGGAAGACCAU | AUGGUCUUCCCAGGUAUAA | Rat, Ms, Mnk | [1376-1394] |
| 406 | GUUGAGCCUUUCUAGAUGG | CCAUCUAGAAAGGCUCAAC | | [2701-2719] |
| 407 | UUUUGACUUCCUUUGCCCU | AGGGCAAAGGAAGUCAAAA | Mnk | [2369-2387] |
| 408 | AUUGCCUUCUGAAGAGGAG | CUCCUCUUCAGAAGGCAAU | Mnk | [2497-2515] |
| 409 | ACUACUUUACCAGCUUCUG | CAGAAGCUGGUAAAGUAGU | Mnk | [616-634] |
| 410 | UGUGCUUUGUGGAAGACCC | GGGUCUUCCACAAAGCACA | Mnk | [1291-1309] |
| 411 | ACCCGUUGAACAACUCUGG | CCAGAGUUGUUCAACGGGU | Mnk | [1084-1102] |
| 412 | AUCUCUAGAGAGUUUGAGC | GCUCAAACUCUCUAGAGAU | Mnk | [2465-2483] |
| 413 | AGUGUUCUUACCUGGUAGG | CCUACCAGGUAAGAACACU | Mnk | [724-742] |
| 414 | GAACCUCAAGGUCUAUAUC | GAUAUAGACCUUGAGGUUC | Mnk | [1610-1628] |
| 415 | GUUCCCUAAAAGCCUGUUC | GAACAGGCUUUUAGGGAAC | Mnk | [2308-2326] |
| 416 | UUUGGCUUUGCUACCAGUU | AACUGGUAGCAAAGCCAAA | Mnk | [3424-3442] |
| 417 | ACAUUGCCAUCAUGGCUGC | GCAGCCAUGAUGGCAAUGU | Ms, Mnk | [694-712] |
| 418 | AAAUAAACGUUCGCUGAGG | CCUCAGCGAACGUUUAUUU | Mnk | [3455-3473] |
| 419 | GGUGACCAUGGCUACAUUG | CAAUGUAGCCAUGGUCACC | Mnk | [2800-2818] |
| 420 | CAGCUGCUGGAUGAGAAGU | ACUUCUCAUCCAGCAGCUG | Rat, Ms, Mnk | [1434-1452] |
| 421 | UUCCCUAAAAGCCUGUUCU | AGAACAGGCUUUUAGGGAA | Mnk | [2309-2327] |
| 422 | AGAGCUCAAGGACUACCUG | CAGGUAGUCCUUGAGCUCU | | [383-401] |
| 423 | UUGCUACCAGUUCCAUAUG | CAUAUGGAACUGGUAGCAA | Mnk | [3431-3449] |
| 424 | UCAUUACCUCCUACUUGCC | GGCAAGUAGGAGGUAAUGA | Mnk | [2240-2258] |
| 425 | GAUUAUACCUGGGAAGACC | GGUCUUCCCAGGUAUAAUC | Rat, Ms, Mnk | [1374-1392] |
| 426 | CAGGGUUCUUGUUUGGACC | GGUCCAAACAAGAACCCUG | Mnk | [2761-2779] |
| 427 | GUAAAUGUUCACUGGGUGG | CCACCCAGUGAACAUUUAC | | [3369-3387] |
| 428 | GCCAACAGCAAGCGGAUUU | AAAUCCGCUUGCUGUUGGC | Mnk | [2911-2929] |
| 429 | UGCCAACAGCAAGCGGAUU | AAUCCGCUUGCUGUUGGCA | Mnk | [2910-2928] |
| 430 | ACUGCGUCUUUGGCAUCAG | CUGAUGCCAAAGACGCAGU | Ms, Mnk | [1546-1564] |
| 431 | AGUAGACAACCUGGCAGUG | CACUGCCAGGUUGUCUACU | Mnk | [578-596] |
| 432 | CUUCUCUUGCCCUUCUUCU | AGAAGAAGGGCAAGAGAAG | | [3113-3131] |
| 433 | AUCCUAGUUCAGUUUUUUG | CAAAAAACUGAACUAGGAU | Mnk | [2355-2373] |
| 434 | UAAUGGUGUGAGGCGUCAG | CUGACGCCUCACACCAUUA | Mnk | [2408-2426] |
| 435 | GGUCUAUAUCAAGACAGUG | CACUGUCUUGAUAUAGACC | Mnk | [1619-1637] |
| 436 | CUACUUUACCAGCUUCUGG | CCAGAAGCUGGUAAAGUAG | Mnk | [617-635] |
| 437 | UUUUCCUCACUGGAAUGGA | UCCAUUCCAGUGAGGAAAA | Mnk | [1945-1963] |
| 438 | UGGGAAGACCAUGGCUACU | AGUAGCCAUGGUCUUCCCA | Rat, Ms, Mnk | [1383-1401] |
| 439 | UAGAUUAUAAAUCCUCUUU | AAAGAGGAUUUAUAAUCUA | Mnk | [2523-2541] |
| 440 | CUUAAUGGUGUGAGGCGUC | GACGCCUCACACCAUUAAG | Mnk | [2406-2424] |
| 441 | CACCCGAAGAAUGUACAAC | GUUGUACAUUCUUCGGGUG | Mnk | [1658-1676] |
| 442 | AACCUCUUCUGGAGGCACU | AGUGCCUCCAGAAGAGGUU | Mnk | [1674-1692] |
| 443 | CAGUUCAUUGCACUGACUC | GAGUCAGUGCAAUGAACUG | Mnk | [1964-1982] |
| 444 | ACCCUUUUGUGUCCCAUGC | GCAUGGGACACAAAAGGGU | | [1864-1882] |
| 445 | UAAACGUUCGCUGAGGUUU | AAACCUCAGCGAACGUUUA | Mnk | [3458-3476] |
| 446 | UUCCCUUUUCUCCAUGCUU | AAGCAUGGAGAAAAGGGAA | | [2390-2408] |
| 447 | AUGUUCACUGGGUGGGUAG | CUACCCACCCAGUGAACAU | | [3373-3391] |
| 448 | UGCUGGUCACAUUGCCUUC | GAAGGCAAUGUGACCAGCA | Mnk | [2487-2505] |
| 449 | AACCUCAAGGUCUAUAUCA | UGAUAUAGACCUUGAGGUU | Mnk | [1611-1629] |
| 450 | UUUAUUUUGGUCCUUUAUG | CAUAAAGGACCAAAAUAAA | Mnk | [2539-2557] |
| 451 | ACUGACUCUGGGAUCUCAG | CUGAGAUCCCAGAGUCAGU | Mnk | [1975-1993] |
| 452 | GUUUGAGCCUUUGCUGGUC | GACCAGCAAAGGCUCAAAC | Mnk | [2476-2494] |
| 453 | AGCCUAUAGCCUCACCUAC | GUAGGUGAGGCUAUAGGCU | Mnk | [1460-1478] |
| 454 | AGAUCCUAAGGGACCACAC | GUGUGGUCCCUUAGGAUCU | Mnk | [2025-2043] |
| 455 | GAGAGGGAGAAUUCUGUUC | GAACAGAAUUCUCCCUCUC | | [2600-2618] |
| 456 | AUGGCUUAGGUGGCACCAU | AUGGUGCCACCUAAGCCAU | | [2716-2734] |
| 457 | CUUUGCUGGUCACAUUGCC | GGCAAUGUGACCAGCAAAG | Mnk | [2484-2502] |
| 458 | ACUUCCUUUGCCCUUUUUC | GAAAAAGGGCAAAGGAAGU | | [2374-2392] |
| 459 | UCCACGUGAACUUGCUGCU | AGCAGCAAGUUCACGUGGA | | [1711-1729] |
| 460 | GAGAAGGUCCACGUGAACU | AGUUCACGUGGACCUUCUC | | [1704-1722] |
| 461 | CUCGGAAUUAAUGUGCCAC | GUGGCACAUUAAUUCCGAG | | [2101-2119] |
| 462 | AUCCCAGUAUCUCAUCUGU | ACAGAUGAGAUACUGGGAU | Mnk | [3000-3018] |
| 463 | UUUCUGUAGCAAAUGACUG | CAGUCAUUUGCUACAGAAA | Mnk | [3084-3102] |
| 464 | UCCUCACUGGAAUGGACAG | CUGUCCAUUCCAGUGAGGA | Mnk | [1948-1966] |
| 465 | GAUGACUGUAAAUGUUCAC | GUGAACAUUUACAGUCAUC | | [3362-3380] |
| 466 | UAUUAGAUUAUAAAUCCUC | GAGGAUUUAUAAUCUAAUA | Mnk | [2520-2538] |
| 467 | CAUGUGAUGACUGUAAAUG | CAUUUACAGUCAUCACAUG | | [3357-3375] |
| 468 | UAUCUCUAGAGAGUUUGAG | CUCAAACUCUCUAGAGAUA | Mnk | [2464-2482] |
| 469 | GUGGAAGACCCUACUUUCG | CGAAAGUAGGGUCUUCCAC | Mnk | [1299-1317] |
| 470 | UUUCUAGAUGGCUUAGGUG | CACCUAAGCCAUCUAGAAA | | [2709-2727] |
| 471 | UUUGUGGAAGACCCUACUU | AAGUAGGGUCUUCCACAAA | Mnk | [1296-1314] |
| 472 | CCUUUGCUGGUCACAUUGC | GCAAUGUGACCAGCAAAGG | Mnk | [2483-2501] |
| 473 | AGUUCCCUAAAAGCCUGUU | AACAGGCUUUUAGGGAACU | Mnk | [2307-2325] |
| 474 | UCUGUAGCAAAUGACUGUG | CACAGUCAUUUGCUACAGA | Mnk | [3086-3104] |
| 475 | AAAGCCCUCCCUGCAAGAA | UUCUUGCAGGGAGGGCUUU | Mnk | [3288-3306] |
| 476 | ACUGAAGAAAGGCAAGGGC | GCCCUUGCCUUUCUUCAGU | Mnk | [3169-3187] |
| 477 | GUUUUUUGACUUCCUUUGC | GCAAAGGAAGUCAAAAAAC | Mnk | [2366-2384] |
| 478 | AUCAGUGUUCUUACCUGGU | ACCAGGUAAGAACACUGAU | Mnk | [721-739] |
| 479 | UAGUUCAGUUUUUUGACUU | AAGUCAAAAAACUGAACUA | Mnk | [2359-2377] |
| 480 | GAGAAUUCUGUUCUCCCAG | CUGGGAGAACAGAAUUCUC | | [2606-2624] |
| 481 | AAUGUACAACCUCUUCUGG | CCAGAAGAGGUUGUACAUU | Mnk | [1667-1685] |
| 482 | GAAUUACGACUUCUCUUGC | GCAAGAGAAGUCGUAAUUC | | [3104-3122] |
| 483 | UCCUACUUGCCAUUCACCC | GGGUGAAUGGCAAGUAGGA | Mnk | [2248-2266] |
| 484 | UAAAUCCUCUUUAUUUUGG | CCAAAAUAAAGAGGAUUUA | Mnk | [2530-2548] |
| 485 | UGCCCGAAUCCUAGUUCAG | CUGAACUAGGAUUCGGGCA | Mnk | [2348-2366] |
| 486 | AGAGAGUUUGAGCCUUUGC | GCAAAGGCUCAAACUCUCU | Mnk | [2471-2489] |
| 487 | CACCUACAAUACCAUCGCC | GGCGAUGGUAUUGUAGGUG | Mnk | [1472-1490] |
| 488 | AUUUCCAGAUUUCAUUACC | GGUAAUGAAAUCUGGAAAU | Mnk | [2229-2247] |
| 489 | UCCUCCUUCGUGUUUGGCU | AGCCAAACACGAAGGAGGA | Mnk | [975-993] |
| 490 | UCCAGAUUUCAUUACCUCC | GGAGGUAAUGAAAUCUGGA | Mnk | [2232-2250] |
| 491 | AUGCACAGUGGUGUGGACA | UGUCCACACCACUGUGCAU | Mnk | [1491-1509] |
| 492 | CUUCUAGCAGUCUGUGCCU | AGGCACAGACUGCUAGAAG | | [3127-3145] |
| 493 | UAGCCUCACCUACAAUACC | GGUAUUGUAGGUGAGGCUA | Mnk | [1466-1484] |
| 494 | UUUGAGCCUUUGCUGGUCA | UGACCAGCAAAGGCUCAAA | Mnk | [2477-2495] |
| 495 | ACUACAUUGCCAUCAUGGC | GCCAUGAUGGCAAUGUAGU | Ms, Mnk | [691-709] |
| 496 | ACGUUCGCUGAGGUUUUGU | ACAAAACCUCAGCGAACGU | Mnk | [3461-3479] |
| 497 | UGUUGUGGCCUUCCUGAAC | GUUCAGGAAGGCCACAACA | Mnk | [2123-2141] |
| 498 | CUUUUCUCCAUGCUUAAUG | CAUUAAGCAUGGAGAAAAG | Mnk | [2394-2412] |
| 499 | UGGCUUUGCUACCAGUUCC | GGAACUGGUAGCAAAGCCA | Mnk | [3426-3444] |
| 500 | UUUGACUUCCUUUGCCCUU | AAGGGCAAAGGAAGUCAAA | Mnk | [2370-2388] |

TABLE B15

| | Sense | Antisense |
|---|---|---|
| 1 | GAAUCACAGAAACACUUUU | AAAAGUGUUUCUGUGAUUC |
| 2 | CCGUGGUCUAUAUCGAGAU | AUCUCGAUAUAGACCACGG |
| 3 | GACAUGGGUUUCUUGGUAA | UUACCAAGAAACCCAUGUC |
| 4 | CCGAGACAGAGGGUUAAAU | AUUUAACCCUCUGUCUCGG |
| 5 | GAUGGUACAAAAUGCUGAA | UUCAGCAUUUUGUACCAUC |
| 6 | CGUGGUCUAUAUCGAGAUC | GAUCUCGAUAUAGACCACG |
| 7 | GCCGUGGUCUAUAUCGAGA | UCUCGAUAUAGACCACGGC |
| 8 | GUGCUGCUCUUUGUGGUGU | ACACCACAAAGAGCAGCAC |
| 9 | AGGAUUCAGACUAAGUUUG | CAAACUUAGUCUGAAUCCU |
| 10 | GGUGAAAACUUCUGCUUGA | UCAAGCAGAAGUUUUCACC |
| 11 | CCUGCUCUGAUUUCCUCCU | AGGAGGAAAUCAGAGCAGG |
| 12 | UGAGUGCGGUUGCUGACAU | AUGUCAGCAACCGCACUCA |
| 13 | AGGGUGAAAACUUCUGCUU | AAGCAGAAGUUUUCACCCU |
| 14 | GUCAGGUGCUGCUCUUUGU | ACAAAGAGCAGCACCUGAC |
| 15 | GUGAUGUGAUUUUGGCCAU | AUGGCCAAAAUCACAUCAC |
| 16 | GCCUGGUGAUGUGAUUUUG | CAAAAUCACAUCACCAGGC |
| 17 | CUGCCGUGGUCUAUAUCGA | UCGAUAUAGACCACGGCAG |
| 18 | CAUCUUUUGUGGGCAGUUA | UAACUGCCCACAAAAGAUG |
| 19 | GGUGAUGUGAUUUUGGCCA | UGGCCAAAAUCACAUCACC |
| 20 | GCUGCUGCCAUCUUUUGUG | CACAAAAGAUGGCAGCAGC |
| 21 | ACCAUCCUGACCUCCUAUU | AAUAGGAGGUCAGGAUGGU |
| 22 | UGGUACAAAAUGCUGAAGA | UCUUCAGCAUUUUGUACCA |
| 23 | GAGGGUGAAAACUUCUGCU | AGCAGAAGUUUUCACCCUC |
| 24 | GCCUCAGAGAACUCUGGAA | UUCCAGAGUUCUCUGAGGC |
| 25 | AGGUCACAGAAUGAAUAGA | UCUAUUCAUUCUGUGACCU |
| 26 | UUGCUGACAUGGGUUUCUU | AAGAAACCCAUGUCAGCAA |
| 27 | CCAUAGUGCAUGGUCUGAU | AUCAGACCAUGCACUAUGG |
| 28 | CUGCUGACGUCAGGAACUU | AAGUUCCUGACGUCAGCAG |
| 29 | CUCUGAAGAAUCACAGAAA | UUUCUGUGAUUCUUCAGAG |
| 30 | CGCUGAGGAUUCAGACUAA | UUAGUCUGAAUCCUCAGCG |
| 31 | CCCGGAGUCAGUACAACUU | AAGUUGUACUGACUCCGGG |
| 32 | ACGCUGAGGAUUCAGACUA | UAGUCUGAAUCCUCAGCGU |
| 33 | GCUCUGAAGAAUCACAGAA | UUCUGUGAUUCUUCAGAGC |
| 34 | AGGGUUAAAUGAACCAGUG | CACUGGUUCAUUUAACCCU |
| 35 | GUGCACUUCUGAAGGACUU | AAGUCCUUCAGAAGUGCAC |
| 36 | CCUCAGAGAACUCUGGAAC | GUUCCAGAGUUCUCUGAGG |
| 37 | GAGGGUUAAAUGAACCAGU | ACUGGUUCAUUUAACCCUC |
| 38 | CCAAGAGUAUGAGGCUCCU | AGGAGCCUCAUACUCUUGG |
| 39 | CAGAAUGAAUAGAUCACCA | UGGUGAUCUAUUCAUUCUG |
| 40 | UCCCAUGCUUGGCUACAGA | UCUGUAGCCAAGCAUGGGA |
| 41 | GGGCUCUGAAGAAUCACAG | CUGUGAUUCUUCAGAGCCC |
| 42 | CUCCUGGGCUCUGAAGAAU | AUUCUUCAGAGCCCAGGAG |
| 43 | GACUAAGUUUGGAAACUCU | AGAGUUUCCAAACUUAGUC |
| 44 | GCACUGCAAAUAACCCAUC | GAUGGGUUAUUUGCAGUGC |
| 45 | ACUGCAAAUAACCCAUCUU | AAGAUGGGUUAUUUGCAGU |
| 46 | GGAUGCCUCAGUGUUAAAA | UUUUAACACUGAGGCAUCC |
| 47 | CACUGCAAAUAACCCAUCU | AGAUGGGUUAUUUGCAGUG |
| 48 | GGGCAAAAAUACAGUCCAA | UUGGACUGUAUUUUUGCCC |
| 49 | UGAUAAGUAACCCUGUAAU | AUUACAGGGUUACUUAUCA |
| 50 | GGAGUAUCAUUGUUUUUGU | ACAAAAACAAUGAUACUCC |
| 51 | GCCUCAUUGAAUUCGCCUA | UAGGCGAAUUCAAUGAGGC |
| 52 | GCAGCUGUCACCAUGUGAU | AUCACAUGGUGACAGCUGC |
| 53 | GGCAAAAAUACAGUCCAAU | AUUGGACUGUAUUUUUGCC |
| 54 | CACCAUGUGAUUUAUUCUU | AAGAAUAAAUCACAUGGUG |
| 55 | CGGGACUAAAAGAAAAGAC | GUCUUUUCUUUUAGUCCCG |
| 56 | GGGACUAAAAGAAAAGACA | UGUCUUUUCUUUUAGUCCC |
| 57 | CCGGGACUAAAAGAAAAGA | UCUUUUCUUUUAGUCCCGG |
| 58 | GGGAGUAUCAUUGUUUUUG | CAAAAACAAUGAUACUCCC |
| 59 | CUCAUUGAAUUCGCCUACA | UGUAGGCGAAUUCAAUGAG |
| 60 | CAUCUCAAAAGAAGUCCAA | UUGGACUUCUUUUGAGAUG |
| 61 | GUGUUAAAAUGACAUCUCA | UGAGAUGUCAUUUUAACAC |
| 62 | CUCAGUGUUAAAAUGACAU | AUGUCAUUUUAACACUGAG |
| 63 | CCUCAGUGUUAAAAUGACA | UGUCAUUUUAACACUGAGG |
| 64 | CCUUAAUUCAGCUGAGUGU | ACACUCAGCUGAAUUAAGG |
| 65 | CCCGGGAGUACAUCUACAU | AUGUAGAUGUACUCCCGGG |
| 66 | CCUCAUUGAAUUCGCCUAC | GUAGGCGAAUUCAAUGAGG |
| 67 | ACCGGGACUAAAAGAAAAG | CUUUUCUUUUAGUCCCGGU |
| 68 | CCUCAAUCGUCUCCUUUAU | AUAAAGGAGACGAUUGAGG |
| 69 | CUGUCUUCAAGGCCAUGUU | AACAUGGCCUUGAAGACAG |
| 70 | ACGUCACACUGCAGGUCAA | UUGACCUGCAGUGUGACGU |
| 71 | AACCGGGACUAAAAGAAAA | UUUUCUUUUAGUCCCGGUU |
| 72 | UGAGGCACUUUUGUUUCUU | AAGAAACAAAAGUGCCUCA |
| 73 | AGAGGAACGAGUGGCGAAU | AUUCGCCACUCGUUCCUCU |
| 74 | CAGUGUUAAAAUGACAUCU | AGAUGUCAUUUUAACACUG |
| 75 | AGCGCUACGAUGUGGAAAC | GUUUCCACAUCGUAGCGCU |
| 76 | ACAGUGUGGAGAGGUAUGA | UCAUACCUCUCCACACUGU |
| 77 | GGUGUCCAUUGAGGGUAUC | GAUACCCUCAAUGGACACC |
| 78 | GAGGCACUUUUGUUUCUUG | CAAGAAACAAAAGUGCCUC |
| 79 | CCUUUGGCAUCAUGAACGA | UCGUUCAUGAUGCCAAAGG |
| 80 | UCCUGCACAACUGUAUCUA | UAGAUACAGUUGUGCAGGA |
| 81 | AGGUGGUGGUGUUGCUUAU | AUAAGCAACACCACCACCU |
| 82 | GCUGUCACCAUGUGAUUUA | UAAAUCACAUGGUGACAGC |
| 83 | GCCUCAGUGUUAAAAUGAC | GUCAUUUUAACACUGAGGC |
| 84 | UGCAUCAACUGGGUCAAGU | ACUUGACCCAGUUGAUGCA |
| 85 | UGGUGGUGUUGCUUAUCUU | AAGAUAAGCAACACCACCA |
| 86 | UGGACAGUUAUUUUGUUGA | UCAACAAAAUAACUGUCCA |
| 87 | UUUGUUUCUUGGGCAAAAA | UUUUUGCCCAAGAAACAAA |
| 88 | GCAAUGAACACCAUCCGAA | UUCGGAUGGUGUUCAUUGC |
| 89 | CCAAGGAAAAUAAAGAACA | UGUUCUUUAUUUUCCUUGG |
| 90 | GUCCUGCACAACUGUAUCU | AGAUACAGUUGUGCAGGAC |
| 91 | UGCCUUAUAACCUGCCUUU | AAAGGCAGGUUAUAAGGCA |
| 92 | GUGCCUUAUAACCUGCCUU | AAGGCAGGUUAUAAGGCAC |
| 93 | GGGAAUUUCUGGUGACAUA | UAUGUCACCAGAAAUUCCC |
| 94 | CCAUGAUAUAAUCUGAAAG | CUUUCAGAUUAUAUCAUGG |
| 95 | GAGAGUCAUUGUUUGCAAU | AUUGCAAACAAUGACUCUC |
| 96 | GAAAAUGGCAGACAAUUUU | AAAAUUGUCUGCCAUUUUC |
| 97 | AGCGGAAAAUGGCAGACAA | UUGUCUGCCAUUUUCCGCU |
| 98 | GGGUUAAAAAACUCAAUGA | UCAUUGAGUUUUUUAACCC |
| 99 | CGGGUUAAAAAACUCAAUG | CAUUGAGUUUUUUAACCCG |
| 100 | CAAGGUUGCAGUGAAUGAU | AUCAUUCACUGCAACCUUG |
| 101 | GACCUCACCAGUGCUUCAU | AUGAAGCACUGGUGAGGUC |
| 102 | CUUUAACCCACGCUUCAAU | AUUGAAGCGUGGGUUAAAG |
| 103 | AAGAAAGACAGUCGGUUUU | AAAACCGACUGUCUUUCUU |
| 104 | GAGCGGAAAAUGGCAGACA | UGUCUGCCAUUUUCCGCUC |
| 105 | UGCGUUAUCUGGGUCUGGA | UCCAGACCCAGAUAACGCA |
| 106 | CCCUCUUGUAAGUCAUCUA | UAGAUGACUUACAAGAGGG |
| 107 | CUGGGAAUUUCUGGUGACA | UGUCACCAGAAAUUCCCAG |
| 108 | AGUACAAUCAUCGGGUUAA | UUAACCCGAUGAUUGUACU |
| 109 | GUCAUUGUUUGCAAUACAA | UUGUAUUGCAAACAAUGAC |
| 110 | GGUUUCAUGUUCACUGUGA | UCACAGUGAACAUGAAACC |
| 111 | UGCAAUACAAAGCUGGAUA | UAUCCAGCUUUGUAUUGCA |
| 112 | CACGCUUCAAUGAGAACAA | UUGUUCUCAUUGAAGCGUG |
| 113 | GGGAAGGGAAGAAAGACAG | CUGUCUUUCUUCCCUUCCC |
| 114 | GCUUCAUAUACCAUGAUAU | AUAUCAUGGUAUAUGAAGC |
| 115 | CUUCAAGGUUGCAGUGAAU | AUUCACUGCAACCUUGAAG |
| 116 | CCACGCUUCAAUGAGAACA | UGUUCUCAUUGAAGCGUGG |
| 117 | UCCCUCUUGUAAGUCAUCU | AGAUGACUUACAAGAGGGA |
| 118 | ACAGUCGGUUUUCCCAUUU | AAAUGGGAAAACCGACUGU |
| 119 | ACGCUUCAAUGAGAACAAC | GUUGUUCUCAUUGAAGCGU |
| 120 | AGUCAUUGUUUGCAAUACA | UGUAUUGCAAACAAUGACU |
| 121 | ACCCACGCUUCAAUGAGAA | UUCUCAUUGAAGCGUGGGU |
| 122 | GAGGGAAUGAUGUUGCCUU | AAGGCAACAUCAUUCCCUC |
| 123 | CUCAAUGAAAUCAGCAAAC | GUUUGCUGAUUUCAUUGAG |
| 124 | CCCAUUUGAAAGUGGGAAA | UUUCCCACUUUCAAAUGGG |
| 125 | GCCACUGAUUGUGCCUUAU | AUAAGGCACAAUCAGUGGC |
| 126 | CCUCUUGUAAGUCAUCUAC | GUAGAUGACUUACAAGAGG |
| 127 | CUAAACCUUACAUGUGUAA | UUACACAUGUAAGGUUUAG |
| 128 | GUGCUUCAUAUACCAUGAU | AUCAUGGUAUAUGAAGCAC |
| 129 | CAGUCGGUUUUCCCAUUUG | CAAAUGGGAAAACCGACUG |
| 130 | GGAAACCCAAACCCUCAAG | CUUGAGGGUUUGGGUUUCC |
| 131 | CUCACUUGUUGCAGUACAA | UUGUACUGCAACAAGUGAG |
| 132 | AUGUUGCCUUCCACUUUAA | UUAAAGUGGAAGGCAACAU |
| 133 | CAGCCAACGAGCGGAAAAU | AUUUUCCGCUCGUUGGCUG |
| 134 | CGCUCCAUGAUGCGUUAUC | GAUAACGCAUCAUGGAGCG |
| 135 | UCGGGUUAAAAAACUCAAU | AUUGAGUUUUUUAACCCGA |
| 136 | CCAUUUGAAAGUGGGAAAC | GUUUCCCACUUUCAAAUGG |
| 137 | GGUUUUCCCAUUUGAAAGU | ACUUUCAAAUGGGAAAACC |
| 138 | GAGUCAUUGUUUGCAAUAC | GUAUUGCAAACAAUGACUC |
| 139 | GAGAAUAUUUCACCCUUCA | UGAAGGGUGAAAUAUUCUC |
| 140 | CCGAGUGGAAGGAAAUUUG | CAAAUUUCCUUCCACUCGG |
| 141 | GACAGAAACACUUUUCGAC | GUCGAAAAGUGUUUCUGUC |
| 142 | ACCUGAAAUUUGCUGGAAU | AUUCCAGCAAAUUUCAGGU |
| 143 | CAGAGAGCUUUUUGAUGAU | AUCAUCAAAAAGCUCUCUG |
| 144 | CACAUGCAAUCUAUCUCAU | AUGAGAUAGAUUGCAUGUG |
| 145 | CCGAGGUGAUCAUUUUAAA | UUUAAAAUGAUCACCUCGG |
| 146 | GUGACCACAUCUUUAAAAU | AUUUUAAAGAUGUGGUCAC |

1. A compound having the following structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide, which may be unmodified or modified, or an unconventional moiety;
wherein in each of (N)x and (N′)y each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
each of x and y is independently an integer between 18 and 40;
wherein (N)x comprises 2′OMe sugar modified and unmodified ribonucleotides, wherein N at the 3′ terminus of (N)x is a 2′OMe sugar modified ribonucleotide, (N)x comprises at least five alternating modified ribonucleotides beginning at the 3′ terminus and at least nine 2′OMe sugar modified ribonucleotides in total and each remaining N is an unmodified ribonucleotide;
wherein in (N′)y comprises at least one unconventional moiety, which unconventional moiety is selected from an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond;
wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA the sequence of which is set forth in SEQ ID NO:4.

2. The compound according to claim 1, wherein x=y=19.

3. The compound according to claim 2, wherein (N)x comprises a 2′-OMe sugar modified ribonucleotide in the first, third, fifth, seventh, ninth, eleventh, thirteenth, fifteenth, seventeenth and nineteenth positions.

4. The compound according to claim 2, wherein (N)x comprises a 2′-OMe sugar modified ribonucleotide in the second, fourth, sixth, eighth, eleventh, thirteenth, fifteenth, seventeenth and nineteenth positions.

5. The compound according to claim 3, wherein (N′)y comprises at least one 2′OMe sugar modified ribonucleotide.

6. The compound according to claim 3, wherein (N′)y comprises at least one DNA nucleotide.

7. The compound according to claim 1, wherein (N′)y comprises at least one unconventional moiety.

8. The compound according to claim 7, wherein the unconventional moiety is an L-DNA moiety.

9. The compound according to claim 8, wherein (N′)y comprises an L-DNA moiety in the 3′ penultimate position.

10. The compound according to claim 9, wherein (N′)y further comprises an L-DNA moiety in the position 5′ and adjacent to the 3′ penultimate L-DNA.

11. The compound according to claim 7, wherein the unconventional moiety is a 2′-5′ linked ribonucleotide.

12. The compound according to claim 11, wherein (N′)y comprises two adjacent 2′-5′ linked ribonucleotides in the 3′ penultimate and 3′ terminal positions.

13. The compound according to claim 12, wherein (N′)y further comprises a 2′-5′ ribonucleotide in the position 5′ and adjacent to the 3′ penultimate 2′-5′ linked ribonucleotide.

14. The compound according to claim 1, wherein (N)x and (N′)y are unphosphorylated or phosphorylated at the 5′ and 3′ termini.

15. The compound according to claim 14, wherein (N)x is unphosphorylated at the 3′ termini.

16. The compound according to claim 1, wherein z″ is present.

17. The compound according to claim 1, wherein (N)x comprises any one of the antisense sequences set forth in any one of SEQ ID Nos: 5571-6391 or 6892-7391.

18. The compound according to claim 17, wherein x=y=19; wherein (N)x comprises a 2′-OMe sugar modified ribonucleotide in the second, fourth, sixth, eighth, eleventh, thirteenth, fifteenth, seventeenth and nineteenth positions; wherein (N′)y comprises unmodified ribonucleotides and an L-DNA at position 17 or 18 or both 17 and 18; and wherein z″ is present and is an abasic moiety.

19. The compound according to claim 17, wherein x=y=19; wherein (N)x comprises a 2′-OMe sugar modified ribonucleotide in the second, fourth, sixth, eighth, eleventh, thirteenth, fifteenth, seventeenth and nineteenth positions; wherein (N′)y comprises unmodified ribonucleotides and at least one 2′-5′ linked ribonucleotide at position 17, 18 or 19.

20. A pharmaceutical composition comprising the compound according to claim 1 and a pharmaceutically acceptable carrier.

21. (canceled)

22. A method for treating a subject suffering from a disease or condition selected from a neurodegenerative disease, a respiratory disorder, an eye disease, a microvascular disorder, a hearing disorder, ischemia reperfusion injury or spinal cord injury or disease or in need of neuroprotection of the optic nerve which comprises administering to the subject the compound of claim 1 in an amount effective to treat the subject.

23-37. (canceled)

38. The composition according to claim 20 formulated for intranasal administration, for systemic administration, for intratracheal administration, or for topical non-invasive administration as an eye drop.

39. A compound having the following structure:

| | |
|---|---|
| 5′ (N)ₓ-Z 3′ | (antisense strand) |
| 3′ Z′-(N′)_{y}-z″ 5′ | (sense strand) |

wherein each of N and N′ is a ribonucleotide which may be unmodified or modified, or an unconventional moiety;
wherein each of (N)x and (N′)y is an oligonucleotide in which each consecutive N or N′ is joined to the next N or N′ by a covalent bond;
wherein Z and Z′ may be present or absent, but if present is independently 1-5 consecutive nucleotides covalently attached at the 3′ terminus of the strand in which it is present;
wherein z″ may be present or absent, but if present is a capping moiety covalently attached at the 5′ terminus of (N′)y;
each of x and y is independently an integer between 18 and 40;
wherein (N)x comprises 2′OMe sugar modified and unmodified ribonucleotides, wherein N at the 3′ terminus of (N)x is a 2′OMe sugar modified ribonucleotide, (N)x comprises at least five alternating modified ribonucleotides beginning at the 3′ terminus and at least nine 2′OMe sugar modified ribonucleotides in total and each remaining N is an unmodified ribonucleotide;
wherein in (N′)y comprises at least one unconventional moiety, which unconventional moiety is selected from an abasic ribose moiety, an abasic deoxyribose moiety, a modified or unmodified deoxyribonucleotide, a mirror nucleotide, and a nucleotide joined to an adjacent nucleotide by a 2′-5′ internucleotide phosphate bond;
wherein the sequence of (N′)_{y} is a sequence having complementarity to (N)x; and wherein the sequence of (N)ₓ comprises an antisense sequence having complementarity to about 18 to about 40 consecutive ribonucleotides in an mRNA the sequence of which is set forth in any one of SEQ ID NO:1-3 or 5-23.

40-41. (canceled)
